(19) United States
(12) Patent Application Publication
(10) Pub. No.: US 2024/0277714 A1
(43) Pub. Date: Aug. 22, 2024

(54) USE OF SOS1 INHIBITORS WITH RAS INHIBITORS TO TREAT CANCERS

(71) Applicant: Revolution Medicines, Inc., Redwood City, CA (US)

(72) Inventors: Grace J. Lee, San Francisco, CA (US); David Church Montgomery, San Mateo, CA (US); Elsa Quintana, San Mateo, CA (US); Christopher J. Schulze, San Mateo, CA (US); Jacqueline Smith, San Mateo, CA (US); David E. Wildes, San Francisco, CA (US)

(21) Appl. No.: 18/554,294

(22) PCT Filed: Apr. 8, 2022
(86) PCT No.: PCT/US22/24027

§ 371 Date: Oct. 6, 2023

Related U.S. Application Data
(60) Provisional application No. 63/172,786, filed on Apr. 9, 2021.

(51) Int. Cl. A61K 31/519; A61K 31/517; A61K 45/06

(52) U.S. Cl. CPC A61K 31/519; A61K 31/517; A61K 45/06

(57) ABSTRACT

The present disclosure relates to combinations of inhibitors of SOS1 and inhibitors of RAS useful in the treatment of diseases or disorders.

### CROSS REFERENCE TO RELATED APPLICATION(S)

This application claims the benefit of priority to U.S. provisional Application Ser. No. 63/172,786, which was filed Apr. 9, 2021, the entire disclosure of which is hereby incorporated by reference as if set forth in its entirety.

### FIELD OF THE DISCLOSURE

The present disclosure relates to combinations of inhibitors of SOS1 and inhibitors of RAS useful in the treatment of diseases or disorders.

### BACKGROUND OF THE DISCLOSURE

It has been well established in literature that RAS proteins (KRAS, HRAS and NRAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression or upstream activation is common in human tumors, and activating mutations in RAS are frequently found in human cancer. For example, activating mutations at codon 12 in RAS proteins function by inhibiting both GTPase-activating protein (GAP)-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13D) and 61 (e.g., Q61K) of RAS are also responsible for oncogenic activity in some cancers.

Despite extensive drug discovery efforts against RAS during the last several decades, a drug directly targeting RAS is still not approved. Additional efforts are needed to uncover additional medicines for cancers driven by the various RAS aberrations and mutations.

### BRIEF SUMMARY

In some aspects, the present disclosure is directed to a method of treating a subject having a RAS protein-related disease or disorder, the method comprising administering to a subject in need of such treatment an SOS1 inhibitor as disclosed herein, and further comprises administering to the subject a therapeutically effective amount of a RAS inhibitor.

### BRIEF DESCRIPTION OF THE DRAWINGS

FIG. 1 is an illustration of the RAS-MAPK pathway. SHP2 activates SOS1, which, in turn, activates RAS, leading to signaling that drives cell growth and survival.

FIGS. 2A (Bliss Score) and 2B (Loewe Score) are three-dimensional surface plots depicting the in vitro combination effect of Compound SOS1-(A) and Compound RAS-(D) observed in NCI-H1355 cells. A synergy score >5 at any point on the plot indicates a positive interaction between the two compounds. These data were obtained according to the method of Example 1.

FIG. 3A (Bliss Score) and 3B (Loewe Score) are three-dimensional surface plots depicting the in vitro combination effect of Compound SOS1-(A) and Compound RAS-(D) observed in NCI-H1944 cells. A synergy score >5 at any point on the plot indicates a positive interaction between the two compounds. These data were obtained according to the method of Example 1.

FIG. 4 (Bliss score) is a three-dimensional plot depicting the in vitro combination effect of Compound SOS1-(A) and Compound RAS-(D) observed in SW948 cells. A synergy score >5 at any point on the plot indicates a positive interaction between the two compounds. These data were obtained according to the method of Example 1.

FIG. 5 (Loewe Score) is a three-dimensional plot depicting the in vitro combination effect of Compound SOS1-(A) and Compound RAS-(D) observed in A−427 cells. A synergy score >5 at any point on the plot indicates a positive interaction between the two compounds. These data were obtained according to the method of Example 1.

FIG. 6 (Bliss Score) is a three-dimensional plot depicting the in vitro combination effect of Compound SOS1-(A) and Compound RAS-(D) observed in LS 180 cells. A synergy score >5 at any point on the plot indicates a positive interaction between the two compounds. These data were obtained according to the method of Example 1.

FIG. 7 (Bliss Score) is a three-dimensional plot depicting the in vitro combination effect of Compound SOS1-(A) and Compound RAS-(D) observed in AsPC-1 cells. A synergy score >5 at any point on the plot indicates a positive interaction between the two compounds. These data were obtained according to the method of Example 1.

FIG. 8 (Bliss Score) is a three-dimensional plot depicting the in vitro combination effect of Compound SOS1-(A) and Compound RAS-(D) observed in HCT 116 cells. A synergy score >5 at any point on the plot indicates a positive interaction between the two compounds. These data were obtained according to the method of Example 1.

FIG. 9 (Bliss Score) is a three-dimensional plot depicting the in vitro combination effect of Compound SOS1-(A) and Compound RAS-(D) observed in SW480 cells. A synergy score >5 at any point on the plot indicates a positive interaction between the two compounds. These data were obtained according to the method of Example 1.

FIG. 10 (Bliss Score) is a three-dimensional plot depicting the in vitro combination effect of Compound SOS1-(A) and Compound RAS-(D) observed in Calu-6 cells. A synergy score >5 at any point on the plot indicates a positive interaction between the two compounds. These data were obtained according to the method of Example 1.

FIG. 11 (Bliss Score) is a three-dimensional plot depicting the in vitro combination effect of Compound SOS1-(A) and Compound RAS-(D) observed in NCI-H1573 cells. A synergy score >5 at any point on the plot indicates a positive interaction between the two compounds. These data were obtained according to the method of Example 1.

FIG. 12 (Bliss Score) is a three-dimensional plot depicting the in vitro combination effect of Compound SOS1-(A) and Compound RAS-(D) observed in LS513 cells. A synergy score >5 at any point on the plot indicates a positive interaction between the two compounds. These data were obtained according to the method of Example 1.

FIG. 13 (Bliss Score) is a three-dimensional plot depicting the in vitro combination effect of Compound SOS1-(A) and Compound RAS-(D) observed in Capan-1 cells. A synergy score >5 at any point on the plot indicates a positive interaction between the two compounds. These data were obtained according to the method of Example 1.

FIG. 14 (Bliss Score) is a three-dimensional plot depicting the in vitro combination effect of Compound SOS1-(A) and Compound RAS-(D) observed in SW837 cells. A synergy score >5 at any point on the plot indicates a positive interaction between the two compounds. These data were obtained according to the method of Example 1.

FIG. 15 (Loewe Score) is a three-dimensional plot depicting the in vitro combination effect of Compound SOS1-(A) and Compound RAS-(D) observed in MIA PaCa-2 cells. A synergy score >5 at any point on the plot indicates a positive interaction between the two compounds. These data were obtained according to the method of Example 1.

FIG. 16A (Bliss Score) and 16B (Loewe Score) are three-dimensional plots depicting the in vitro combination effect of Compound SOS1-(A) and Compound RAS-(D) observed in SW 1116 cells. A synergy score >5 at any point on the plot indicates a positive interaction between the two compounds. These data were obtained according to the method of Example 1.

FIG. 17A (Bliss Score) and 17B (Loewe Score) are three-dimensional plots depicting the in vitro combination effect of Compound SOS1-(A) and Compound RAS-(D) observed in LS123 cells. A synergy score >5 at any point on the plot indicates a positive interaction between the two compounds. These data were obtained according to the method of Example 1.

FIG. 18 (Bliss Score) is a three-dimensional plot depicting the in vitro combination effect of Compound SOS1-(A) and Compound RAS-(D) observed in SW1463 cells. A synergy score >5 at any point on the plot indicates a positive interaction between the two compounds. These data were obtained according to the method of Example 1.

FIG. 19 (Loewe Score) is a three-dimensional plot depicting the in vitro combination effect of BI-3406 (SOS1 inhibitor) and Compound RAS-(A) observed in KYSE-410 cells. A synergy score >5 at any point on the plot indicates a positive interaction between the two compounds. These data were obtained according to the method of Example 2.

FIG. 20 (Bliss Score) is a three-dimensional plot depicting the in vitro combination effect of BI-3406 (SOS1 inhibitor) and Compound RAS-(A) observed in NCI-H358 cells. A synergy score >5 at any point on the plot indicates a positive interaction between the two compounds. These data were obtained according to the method of Example 2.

FIG. 21 (Loewe Score) is a three-dimensional plot depicting the in vitro combination effect of BI-3406 (SOS1 inhibitor) and Compound RAS-(A) observed in SW837 cells. A synergy score >5 at any point on the plot indicates a positive interaction between the two compounds. These data were obtained according to the method of Example 2.

FIGS. 22A and 22B are graphs depicting the in vitro combination effect of SOS1 inhibitor Compound SOS1-(B) and RAS inhibitor Compound RAS-(E) (FIG. 22A) or Compound RAS-(B) (FIG. 22B) observed in SW837 cells. These data were obtained according to the method of Example 3.

FIGS. 23A and 23B are graphs depicting the in vitro combination effect of SOS1 inhibitor Compound SOS1-(B) and RAS inhibitor Compound RAS-(E) (FIG. 23A) or Compound RAS-(B) (FIG. 23B) observed in SW837 cells. These data were obtained according to the method of Example 4.

FIG. 24 is a graph depicting the in vitro combination effect of SOS1 inhibitor Compound SOS1-(B) and RAS inhibitor Compound RAS-(E) observed in AsPC-1 cells. These data were obtained according to the method of Example 5.

FIG. 25 is a graph depicting the in vitro combination effect of SOS1 inhibitor Compound SOS1-(B) and RAS inhibitor Compound RAS-(E) observed in SNU-C2B cells. These data were obtained according to the method of Example 6.

FIG. 26 is a graph depicting the in vitro combination effect of SOS1 inhibitor Compound SOS1-(B) and RAS inhibitor Compound RAS-(E) observed in HCT-15 cells. These data were obtained according to the method of Example 7.

FIG. 27 is a graph showing the in vitro combination effect of SOS1 inhibitor BI-3406 and RAS^{G13C}(ON) inhibitor Compound RAS-(C) observed in NCI-H1355 cells. These data were obtained according to the method of Example 8.

FIG. 28 is a graph showing the in vitro combination effect of SOS1 inhibitor Compound SOS1-(A) and RAS^{G13C}(ON) inhibitor Compound RAS-(C) observed in TOV-21G cells. These data were obtained according to the method of Example 9.

FIG. 29 is a graph showing the efficacy of repeated daily dosing of Compound SOS1-(B) at 100 mg/kg po (tumor growth inhibition, TGI 79%) and Compound RAS-(B) at 100 mg/kg (75%) in a KYSE-410 xenograft model. These data were obtained according to the method of Example 10.

FIG. 30 is graph showing % change in body weight of mice subjected to repeated daily dosing of Compound SOS1-(B) at 100 mg/kg po and Compound RAS-(B) at 100 mg/kg in a KYSE-410 xenograft model. These data were obtained according to the method of Example 10.

FIG. 31 is a graph showing the change in tumor volume in mice subjected to repeated daily dosing of Compound SOS1-(B) at 100 mg/kg po (tumor growth inhibition, TGI 79%) and Compound RAS-(B) at 100 mg/kg (75%) in a KYSE-410 xenograft model. These data were obtained according to the method of Example 10.

FIG. 32A is a graph showing the efficacy of repeated daily dosing of Compound SOS1-(C) at 100 mg/kg po, Compound RAS-(F) at 100 mg/kg po, or a combination of Compound SOS1-(C) and Compound RAS-(F) in a human colorectal cancer (CRC) patient-derived xenograft (PDX) model CRC022. These data were obtained according to the method of Example 11. FIG. 32B is the waterfall plot depicting percentage change in tumor volume for an individual mouse.

FIG. 33 is a graph showing the efficacy of repeated daily dosing of Compound RAS-(F) at 100 mg/kg po, or a combination of Compound SOS1-(C) and Compound RAS-(F) in a human non-small cell lung cancer NCI-H2030 xenograft model. These data were obtained according to the method of Example 12.

### DETAILED DESCRIPTION OF THE DISCLOSURE

The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which the present disclosure belongs. All patents and publications cited in this specification are incorporated herein by reference in their entireties.

### General Methods

The practice of the present disclosure will employ, unless otherwise indicated, conventional techniques of cell culturing, molecular biology (including recombinant techniques), microbiology, cell biology, biochemistry, and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Terms

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise. The use of the term “or” is used to mean “and/or” unless explicitly indicated to refer to alternatives only or the alternative are mutually exclusive, although the disclosure supports a definition that refers to only alternatives and “and/or.”

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²p, ³³p, ³⁵S ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl)per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{o}; —(CH₂)₀₋₄OR^{o}; —O(CH₂)₀₋₄R^{o}; —O—(CH₂)₀₋₄C(O)OR^{o}; —(CH₂)₀₋₄CH(O R^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄—C(O)—N(R^{o})₂; —(CH₂)₀₋₄—C(O)—N(R^{o})—S(O)₂—R^{o}; —C(NCN)NR^{o}₂; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; —SC(S)SR^{o}; —(C H₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N (OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂N R^{o}₂; —N(R^{o})S(O)₂R^{o}; —N(OR^{o})R^{o}; —C(NOR^{o})NR^{o}₂; —C(NH)NR^{o}₂; —P(O)₂R^{o}; —P(O)R^{o}₂; —P(O)(OR^{o})₂; —OP(O)R^{o}₂; —OP(O)(OR^{o})₂; —OP(O)(OR^{o})R^{o}, —SiR⁰₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), may be, independently, halogen, —(CH₂)₀₋₂R^{•}, -(haloR⁵⁰⁰), —(CH₂)₀₋₂OH, —(CH₂)₀₋₂OR^{•}, —(CH₂)₀₋₂CH(OR^{•})₂; —O(halo R^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(C H₂)₀₋₂SH, —(CH₂)₀₋₂NH₂, —(CH₂)₀₋₂NHR^{•}, —(CH₂)₀₋₂NR^{o}₂, —NO₂, —SiR^{o}₃, —OSiR^{o}₃, —C(O)SR ^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cr-Cy alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

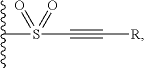

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

An “amino acid substitution,” as used herein, refers to the substitution of a wild-type amino acid of a protein with a non-wild-type amino acid. Amino acid substitutions can result from genetic mutations and may alter one or more properties of the protein (e.g., may confer altered binding affinity or specificity, altered enzymatic activity, altered structure, or altered function). For example, where a RAS protein includes an amino acid substitution at position Y96, this notation indicates that the wild-type amino acid at position 96 of the RAS protein is a Tyrosine (Y), and that the RAS protein including the amino acid substitution at position Y96 includes any amino acid other than Tyrosine (Y) at position 96. The notation Y96D indicates that the wild-type Tyrosine (Y) residue at position 96 has been substituted with an Aspartic Acid (D) residue.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-Cᵤ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

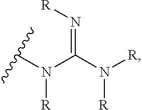

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., a macrocyclic moiety or B) to a second moiety (e.g., W) in a compound of any one of Formula AI, Formula BI, Formula CI, Formula DIA, or a subformula thereof, such that the resulting compound is capable of achieving an IC50 of 2 uM or less in the Ras-RAF disruption assay protocol provided in the Examples below, and provided here:

The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group. The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure,

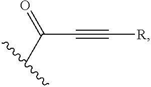

wherein R is any chemically feasible substituent described herein.

As used herein, the term “pharmaceutical composition” refers to a compound, such as a compound of the present disclosure, or a pharmaceutically acceptable salt thereof, formulated together with a pharmaceutically acceptable excipient.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Pharmaceutically acceptable salts of compounds disclosed herein are contemplated by the present invention. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

A “therapeutic regimen” refers to a dosing regimen whose administration across a relevant population is correlated with a desired or beneficial therapeutic outcome.

The disclosure also includes pharmaceutical compositions comprising an effective amount of a disclosed compound and a pharmaceutically acceptable carrier. The term “carrier”, as used in this disclosure, encompasses excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The terms “inhibiting” and “reducing,” or any variation of these terms, includes any measurable or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., SOS1.Ras-family protein binding activity) compared to normal.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

As used herein, the term “dosage form” refers to a physically discrete unit of a compound (e.g., a compound of the present disclosure) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present disclosure) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

A “patient” or “subject” is a mammal, e.g., a human, mouse, rat, guinea pig, dog, cat, horse, cow, pig, or non-human primate, such as a monkey, chimpanzee, baboon or rhesus.

The term “sample” or “biological sample,” as used herein, refers to a sample obtained from a subject, e.g., a human subject or a patient, which may be tested for a particular molecule, for example wild type. Samples may include, but are not limited to, biopsies, tissues, cells, buccal swab sample, body fluids, including blood, serum, plasma, urine, saliva, cerebral spinal fluid, tears, pleural fluid and the like.

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein. In some embodiments, the inhibitor can be nucleic acid molecules including, but not limited to, siRNA that reduce the amount of functional protein in a cell. Accordingly, compounds said to be “capable of inhibiting” a particular protein, e.g., RAS or SOS1, comprise any such inhibitor.

The term “SHP2” means “Src Homology 2 domain-containing protein tyrosine phosphatase 2” and is also known as SH-PTP2, SH-PTP3, Syp, PTP1D, PTP2C, SAP-2 or PTPN11. SHP2 is a non-receptor protein tyrosine phosphatase encoded by the PTPN11 gene that contributes to multiple cellular functions including proliferation, differentiation, cell cycle maintenance and migration. SHP2 is involved in signaling through the RAS-mitogen-activated protein kinase (MAPK), the JAK-STAT and/or the phosphoinositol 3-kinase-AKT pathways. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through RTKs leads to exposure of the catalytic site resulting in enzymatic activation of SHP2. SHP2 can exist in wild-type and mutant forms.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

The term “mutation” as used herein indicates any modification of a nucleic acid and/or polypeptide which results in an altered nucleic acid or polypeptide. The term “mutation” may include, for example, point mutations, deletions of a single or multiple residues in a polynucleotide, or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

The term “SOS” (e.g., a “SOS mutation”) refers to SOS genes, which are known in the art to include RAS guanine nucleotide exchange factor proteins that are activated by receptor tyrosine kinases to promote GTP loading of RAS and signaling. The term SOS includes all SOS homologs that promotes the exchange of Ras-bound GDP by GTP. In particular embodiments, SOS refers specifically to “son of sevenless homolog 1” (“SOS1”). SOS1 is critically involved in the activation of RAS-family protein signaling in cancer via mechanisms other than mutations in RAS-family proteins. SOS1 interacts with the adaptor protein Grb2 and the resulting SOS1/Grb2 complex binds to activated/phosphorylated Receptor Tyrosine Kinases (e.g., EGFR, ErbB2, ErbB3, ErbB4, PDGFR-A/B, FGFR1/2/3, IGF1 R, INSR, ALK, ROS, TrkA, TrkB, TrkC, RET, c-MET, VEGFR1/2/3, AXL) (Pierre et al., Biochem. Pharmacol., 2011, 82(9): 1049-56). SOS1 is also recruited to other phosphorylated cell surface receptors such as the T cell Receptor (TCR), B cell Receptor (BCR) and monocyte colony-stimulating factor receptor (Salojin et al., J. Biol. Chem. 2000, 275(8):5966-75). This localization of SOS1 to the plasma membrane, proximal to RAS-family proteins, enables SOS1 to promote RAS-family protein activation. SOS1-activation of RAS-family proteins can also be mediated by the interaction of SOS1/Grb2 with the BCR-ABL oncoprotein commonly found in chronic myelogenous leukemia (Kardinal et al., 2001, Blood, 98:1773-81; Sini et al., Nat. Cell Biol., 2004, 6(3):268-74). SOS1 is also a GEF for the activation of the GTPases RAC1 (Ras-related C3 botulinum toxin substrate 1) (Innocenti et al., J. Cell Biol., 2002, 156(1):125-36). RAC1, like RAS-family proteins, is implicated in the pathogenesis of a variety of human cancers and other diseases (Bid et al., Mol. Cancer Ther. 2013, 12(10):1925-34). Son of sevenless 2 (SOS2), a homolog of SOS1 in mammalian cells, also acts as a GEF for the activation of RAS-family proteins (Pierre et al., Biochem. Pharmacol., 2011, 82(9): 1049-56; Buday et al., Biochim. Biophys. Acta., 2008, 1786(2):178-87). Published data from mouse knockout models suggests a redundant role for SOS1 and SOS2 in homeostasis in the adult mouse. Whilst germline knockout of SOS1 in mice results in lethality during mid-embryonic gestation (Qian et al., EMBO J., 2000, 19(4):642-54), systemic conditional SOS1 knockout adult mice are viable (Baltanas et al., Mol. Cell. Biol., 2013, 33(22):4562-78). SOS2 gene targeting did not result in any overt phenotype in mice (Esteban et al., Mol. Cell. Biol., 2000, 20(17):6410-3). In contrast, double SOS1 and SOS2 knockout leads to rapid lethality in adult mice (Baltanas et al., Mol. Cell. Biol., 2013, 33(22):4562-78). These published data suggest that selective targeting of individual SOS isoforms (e.g., selective SOS1 targeting) may be adequately tolerated to achieve a therapeutic index between SOS1/RAS-family protein driven cancers (or other SOS1/RAS-family protein pathologies) and normal cells and tissues. Selective pharmacological inhibition of the binding of the catalytic site of SOS1 to RAS-family proteins is expected to prevent SOS1-mediated activation of RAS-family proteins to the GTP-bound form. Such SOS1 inhibitor compounds are be expected to consequently inhibit signaling in cells downstream of RAS-family proteins (e.g., ERK phosphorylation). In cancer cells associated with dependence on RAS-family proteins (e.g., KRAS mutant cancer cell lines), SOS1 inhibitor compounds are be expected to deliver anti-cancer efficacy (e.g., inhibition of proliferation, survival, metastasis, etc.). High potency towards inhibition of SOS1.RAS-family protein binding (nanomolar level IC50 values) and ERK phosphorylation in cells (nanomolar level IC50 values) are desirable characteristics for a SOS1 inhibitor compound. Furthermore, a desirable characteristic of a SOS1 inhibitor compound would be the selective inhibition of SOS1 over SOS2. This conclusion is based on the viable phenotype of SOS1 knockout mice and lethality of SOS1/SOS2 double knockout mice, as described above.

As used herein, a “SOS1 inhibitor” refers to any agent, (e.g., a small molecule (e.g., less than 750 Da)) capable of inhibiting SOS1. SOS1 inhibitors can include selective SOS1 inhibitors and inhibitors that also inhibit other proteins. In some embodiments, SOS1 inhibitors may also inhibit SOS2, with a selectivity ratio less than 10-fold for inhibition of SOS1 relative to SOS2. In some embodiments, SOS1 inhibitors will selectively inhibit SOS1, with a selectivity ratio greater of at least about 10-fold, such as greater than at least about 30-fold, for inhibition of SOS1 relative to SOS2.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The terms “RAS inhibitor” and “inhibitor of [a] RAS” are used interchangeably to refer to any inhibitor that targets a RAS protein. In various embodiments, these terms include RAS(OFF) and RAS(ON) inhibitors such as, e.g., the KRAS(OFF) and KRAS(ON) inhibitors. A RAS inhibitor may be MRTX1133. The term “RAS(OFF) inhibitor” refers to any inhibitor that binds to a RAS protein in its GDP-bound “OFF” position. The term “RAS(ON) inhibitor” refers to any inhibitor that binds to a RAS protein in its GTP-bound “ON” position.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). The term “KRAS(ON) inhibitor” refers to any inhibitor that binds to KRAS in its GTP-bound “ON” position. RAS(ON) inhibitors described herein include compounds of Formula A00, Formula AI, Formula BI, Formula CI, Formula DIa, and subformula thereof, and compounds of Table A1, Table A2, Table B1, Table B2, Table C1, Table C2, Table D1a, Table D1b, Table D2, Table D3, as well as salts (e.g., pharmaceutically acceptable salts), solvates, hydrates, stereoisomers (including atropisomers), and tautomers thereof.

As used herein, the term “RAS(OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

The term “KRAS(OFF) inhibitor” refers to any inhibitor that binds to KRAS in its GDP-bound “OFF” position. Reference to the term KRAS(OFF) inhibitor includes AMG 510 and MRTX849. In some embodiments, reference to the term KRAS(OFF) inhibitor includes any such KRAS(OFF) inhibitor disclosed in any one of the following patent applications: WO 2022066805, WO 2022066646, WO 2022063297, WO 2022061251, WO 2022056307, WO 2022052895, WO 2022047093, WO 2022042630, WO 2022040469, WO 2022037560, WO 2022031678, WO 2022028492, WO 2022028346, WO 2022026726, WO 2022026723, WO 2022015375, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223, each of which are incorporated herein by reference in its entirety.

As used herein, the term “RAS(ON)^{MULTI} inhibitor” refers to a RAS(ON) inhibitor of at least 3 RAS variants with missense mutations at one of the following positions: 12, 13, 59, 61, or 146. In some embodiments, a RAS(ON)^{MULTI} inhibitor refers to a RAS(ON) inhibitor of at least 3 RAS variants with missense mutations at one of the following positions: 12, 13, and 61.

In any embodiment herein regarding a RAS(OFF) inhibitor, such RAS(OFF) inhibitor may be substituted by a RAS inhibitor disclosed in the following patent publication: WO 2021041671, which is incorporated herein by reference in its entirety. In some embodiments, such a substituted RAS inhibitor is MRTX1133.

Exemplary RAS(OFF) inhibitors include the following, without limitation:

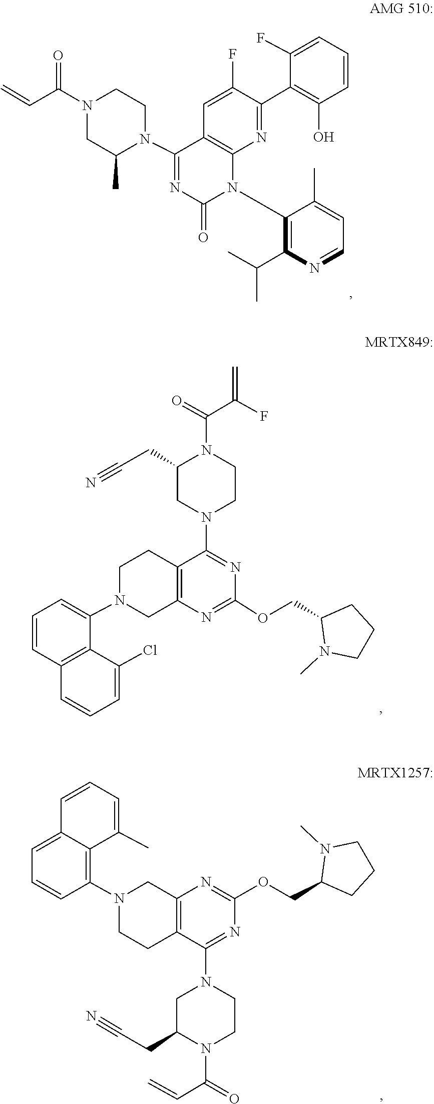

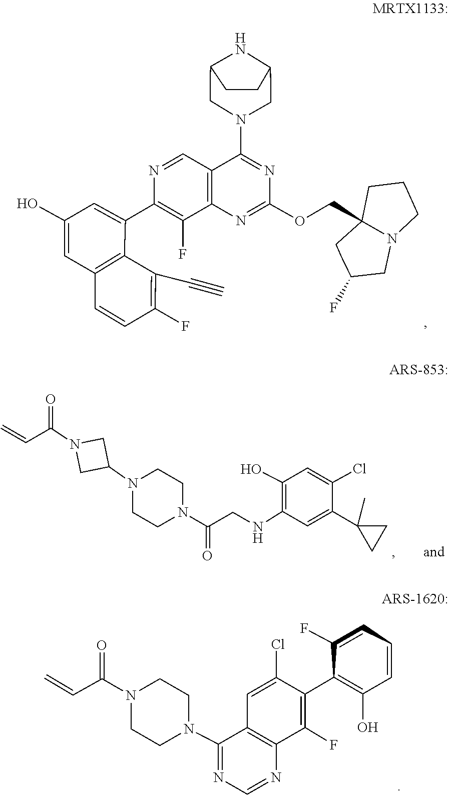

Reference to a “subtype” of a cell (e.g., a KRAS^{G12C} subtype, a KRAS^{G12S} subtype, a KRAS^{G12D} subtype, a KRAS^{G12V} subtype) means that the cell contains a gene mutation encoding a change in the protein of the type indicated. For example, a cell classified as a “KRAS^{G12C} subtype” contains at least one KRAS allele that encodes an amino acid substitution of cysteine for glycine at position 12 (^{G12}C); and, similarly, other cells of a particular subtype (e.g., KRAS^{G12D}, KRAS^{G12S} and KRAS^{G12V} subtypes) contain at least one allele with the indicated mutation (e.g., a KRAS^{G12D} mutation, a KRAS^{G12S} mutation or a KRAS^{G12V} mutation, respectively). Unless otherwise noted, all amino acid position substitutions referenced herein (such as, e.g., “^{G12C}” in KRAS^{G12C}) correspond to substitutions in the human version of the referenced protein, i.e., KRAS^{G12C} refers to a G→C substitution in position 12 of human KRAS.

The term “monotherapy” refers to a method of treatment comprising administering to a subject a single therapeutic agent, optionally as a pharmaceutical composition. For example, a monotherapy may comprise administration of a pharmaceutical composition comprising a therapeutic agent and one or more pharmaceutically acceptable carrier, excipient, diluent, and/or surfactant. The therapeutic agent may be administered in an effective amount. The therapeutic agent may be administered in a therapeutically effective amount.

The term “combination therapy” refers to a method of treatment comprising administering to a subject at least two therapeutic agents, optionally as one or more pharmaceutical compositions. For example, a combination therapy may comprise administration of a single pharmaceutical composition comprising at least two therapeutic agents and one or more pharmaceutically acceptable carrier, excipient, diluent, and/or surfactant. A combination therapy may comprise administration of two or more pharmaceutical compositions, each composition comprising one or more therapeutic agent and one or more pharmaceutically acceptable carrier, excipient, diluent, and/or surfactant. In various embodiments, at least one of the therapeutic agents is a SOS1 inhibitor. In various embodiments, at least one of the therapeutic agents is a RAS inhibitor. The two agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due an additive or synergistic effect of combining the two or more therapeutics.

### Methods of Treatment

Disruption of the RAS/MAPK signaling pathway is a common driver of abnormal growth and proliferation in many types of cancer and has also been implicated in developmental diseases such as Noonan Syndrome. Oncogenic hyper-activation of this pathway can occur through alterations in the levels of active GTP-bound RAS and inactive GDP-bound RAS, such as mutations resulting in disruption of RAS guanine nucleotide exchange factors (GEFs) and GTPase-activating proteins (GAPs). SHP2 is a non-receptor protein tyrosine phosphatase encoded by the PTPN11 gene that functions upstream of RAS. SHP2, e.g., wild-type SHP2, can regulate RAS signaling through activation of SOS1, a GEF that converts inactive RAS-GDP to RAS-GTP. The development of inhibitors targeting either SHP2, SOS1, or RAS is an emerging and attractive approach toward treatment of RAS-driven cancers, and several such candidates are currently undergoing clinical trials.

The present invention is directed to a method of treating a subject having a RAS protein-related disease or disorder, the method comprising administering to a subject in need of such treatment an SOS1 inhibitor as disclosed herein, and further comprises administering to the subject a therapeutically effective amount of a RAS inhibitor selected from the group consisting of a RAS(ON) inhibitor and a RAS(OFF) inhibitor, and a combination thereof. In some embodiments, the SOS1 inhibitor targets a disease or disorder mediated by wild-type SHP2 protein. In some embodiments, the RAS inhibitor targets a wild-type RAS protein. In some embodiments, the RAS protein is KRAS. In some embodiments, the RAS inhibitor targets a RAS protein mutation. In some embodiments, the RAS protein mutation is at a position selected from the group consisting of G12, G13, Q61, A146, K117, L19, Q22, V14, A59, and a combination thereof. In some embodiments, the mutation is selected from the group consisting of G12, G13, and Q61. In some embodiments, the mutation is selected from the group consisting of G12C, G12D, G12A, G12S, G12V, G13C, G13D, Q61K, and Q61L.

According to embodiments of the present invention, the method comprises administering to the subject a therapeutically effective amount of a SOS1 inhibitor as disclosed herein, and further comprises administering to the subject a therapeutically effective amount of a RAS inhibitor selected from the group consisting of a RAS(ON) inhibitor and a RAS(OFF) inhibitor, and a combination thereof in the treatment of a tumor or cancer.

Accordingly, also provided is a method of treating cancer in a subject in need thereof, the method comprising administering to the subject a therapeutically effective amount of a SOS1 inhibitor as disclosed herein, and may further comprise administering to the subject a therapeutically effective amount of a RAS inhibitor as disclosed herein. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma.

In some embodiments, methods provided herein may be used for the treatment of a wide variety of cancers including tumors such as lung, prostate, breast, brain, skin, cervical carcinomas, testicular carcinomas, etc. More particularly, cancers that may be treated include, but are not limited, to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors,
Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms, multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the disease or disorder is selected from the group consisting of tumors of hematopoietic and lymphoid system; a myeloproliferative syndrome; a myelodysplastic syndromes; leukemia; acute myeloid leukemia; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian serous cystadenocarcinoma; paraganglioma; pheochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer (e.g., small and/or large intestinal cancer); thyroid cancer; endometrial cancer; cancer of the biliary tract; soft tissue cancer; ovarian cancer; central nervous system cancer (e.g., primary CNS lymphoma); stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma. In some embodiments, the disease or disorder is selected from brain glioblastoma (GBM), lung adenocarcinoma, colon adenocarcinoma (CRC), bone marrow leukemia, acute myelocytic leukemia (AML), breast carcinoma (NOS), unknown primary melanoma, non-small cell lung carcinoma (NOS), skin melanoma, breast invasive ductal carcinoma (IDC), lung squamous cell carcinoma (SCC), unknown primary adenocarcinoma, bone marrow multiple myeloma, gastroesophageal junction adenocarcinoma, bone marrow myelodysplastic syndrome (MDS), prostate acinar adenocarcinoma, bladder urothelial (transitional cell) carcinoma, uterus endometrial adenocarcinoma (NOS), bone marrow leukemia B cell acute (B-ALL), stomach adenocarcinoma (NOS), and unknown primary carcinoma (NOS). In some embodiments, the disease or disorder is selected from brain glioblastoma (GBM), lung adenocarcinoma, colon adenocarcinoma (CRC), bone marrow leukemia non-lymphocytic acute myelocytic (AML) and breast carcinoma (NOS).

In some embodiments, the cancer is lung cancer, pancreatic cancer, or colorectal cancer. In some embodiments, the cancer is lung cancer. In some embodiments, the cancer is lung cancer comprising a mutation at STK11, Keap1, or a combination of STK11 and Keap1. In some embodiments, the cancer is pancreatic cancer. In some embodiments, the cancer is colorectal cancer.

### SOS1 Inhibitors

In some embodiments, the SOS1 inhibitor is selected from those disclosed in WO 2018/115380, WO 2018/172250, WO 2019/122129, and WO 2019/201848, the disclosures of each of which are hereby incorporated by reference as if set forth in their entirety. In some embodiments, the SOS1 inhibitor is selected from those disclosed in U.S. provisional Ser. No. 63/031,318, PCT/US2020/059024, WO 2021092115, PCT/US2020/020602, WO 2020180768 and PCT/US2020/020609, WO 2020180770, the disclosures of each of which are hereby incorporated by reference as if set forth in their entirety. In some embodiments, the SOS1 inhibitor is selected from those disclosed in WO 2022061348, WO 2022058344, WO 2022028506, WO 2022026465, WO 2022017339, WO 2022017519, WO 2021259972, WO 2021249519, WO 2021249475, WO 2021228028, WO 2021225982, WO 2021203768, WO 2021173524, WO 2021130731, WO 2021127429, WO 2021105960, WO 2021074227, WO 2020173935, WO 2020146470, WO 2019201848, WO 2019/122129, WO 2018172250, WO 2018115380, CN 113912608, CN 113801114, CN 113200981, US 20210338694, and U.S. Pat. No. 8,232,283.

In some embodiments, the SOS1 inhibitor is a compound having the structure of Formula (41-I),

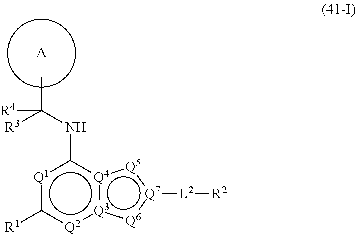

or a pharmaceutically acceptable salt, solvate, isomer, prodrug, or tautomer thereof, wherein:
Q¹ and Q² are independently CH or N;
Q³, Q⁴, and Q⁷ are independently C or N, wherein at least one of Q³ and Q⁴ is C and wherein Q³, Q⁴, and Q⁷ are not all N;
Q⁵ is CH, N, NH, O, or S;
Q⁶ is CH, N, NH, N—C₁₋₆ alkyl, N—C₁₋₆ heteroalkyl, N-(3-7 membered cycloalkyl), N-(3-7 membered heterocyclyl), O, or S;
wherein at least one of Q¹, Q², Q³, Q⁴, Q⁵, Q⁶, and Q⁷ is N, NH, O, or S;
R¹ is selected from the group consisting of H, C₁₋₆ alkyl, halogen, —NHR^{1a}, —OR^{1a}, cyclopropyl, and —CN; wherein C₁₋₆ alkyl is optionally substituted with halogen, —NHR^{1a}, or —OR^{1a}; wherein R^{1a} is H, C₁₋₆ alkyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

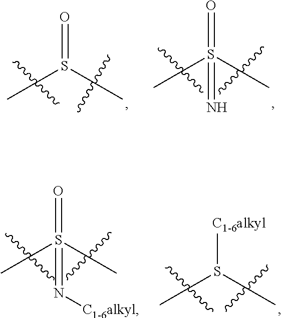

C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, C₂₋₆ alkenyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, —OH, —OR^{2a}, oxo, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2e} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl.

In some embodiments, the present invention is directed to a method of treating a subject having a disease or disorder characterized by SHP2-mediated activation of a RAS protein comprising administering to a subject in need of such treatment a therapeutically effective amount of a SOS1 inhibitor having the structure of Formula (41-I), or a pharmaceutically acceptable salt, solvate, hydrate, tautomer, or isomer thereof.

In some embodiments, the method of inhibiting SOS1 in a subject comprises administering to the subject in need of such treatment a therapeutically effective amount of a SOS1 inhibitor having the structure of Formula (41-I), or a pharmaceutically acceptable salt, solvate, hydrate, tautomer, or isomer thereof.

In some embodiments, the present invention is directed to a method of inhibiting the interaction of SOS1 and a RAS-family protein in a cell or inhibiting the interaction of SOS1 and RAC1 in a cell, comprising administering to the cell a SOS1 inhibitor having the structure of Formula (41-I), or a pharmaceutically acceptable salt, solvate, hydrate, tautomer, or isomer thereof.

In some embodiments, the present invention is directed to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof a therapeutically effective amount of a SOS1 inhibitor having the structure of Formula (41-I), or a pharmaceutically acceptable salt, solvate, hydrate, tautomer, or isomer thereof.

In some embodiments, the present invention is directed to a method of treating or preventing cancer in a subject in need thereof, comprising administering to the subject a therapeutically effective amount of a SOS1 inhibitor having the structure of Formula (41-I), or a pharmaceutically acceptable salt, solvate, hydrate, tautomer, or isomer thereof.

In some embodiments, the SOS1 inhibitor is a compound having the structure of Formula (41-I-a),

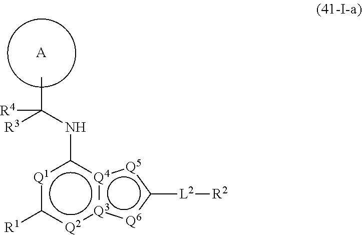

or a pharmaceutically acceptable salt, solvate, isomer, prodrug, or tautomer thereof, wherein:
Q¹, Q², Q⁵ and A are as defined in Formula (41-I);
Q³ and Q⁴ are independently C or N, wherein at least one of Q³ and Q⁴ is C;
Q⁶ is CH, N, NH, O, or S;
wherein at least one of Q¹, Q², Q³, Q⁴, Q⁵, and Q⁶ is N, NH, O, or S;
R¹ is selected from the group consisting of H, halogen, C₁₋₆ alkyl, cyclopropyl, —CN, and —OR^{1a}; wherein R^{1a} is H or C₁₋₆ alkyl;
L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6;
R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c}, is H or C₁₋₆ alkyl; and
R³ and R⁴ are independently H or C₁₋₆ alkyl; wherein at least one of R³ and R⁴ is not H; or R³ and R⁴ together with the atom to which they are attached combine to form a 3-6 membered cycloalkyl.

In some embodiments, the SOS1 inhibitor is a compound having the structure of Formula (41-II),

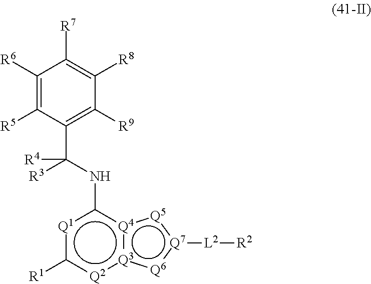

or a pharmaceutically acceptable salt, solvate, isomer, prodrug, or tautomer thereof, wherein:
L², Q¹, Q², Q³, Q⁴, Q⁵, Q⁶, Q⁷, R¹, R², R³ and R⁴ are as defined in Formula (41-I);
R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹²—NR¹⁰S(O)₂R¹¹, —S(O)NR¹¹R¹², —S(O)R¹⁰, —NR¹⁰S(O)NR¹¹R¹², —NR^{10S}(O)R¹¹, 3-14 membered heterocyclyl, 6-10 membered aryl, or 5-10 membered heteroaryl;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the SOS1 inhibitor is a compound having the structure of Formula (41-III),

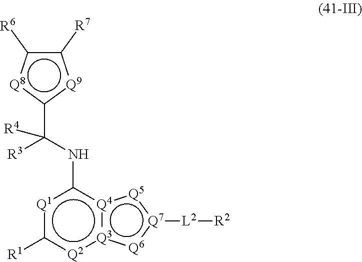

or a pharmaceutically acceptable salt, solvate, isomer, prodrug, or tautomer thereof, wherein:
L², Q¹, Q², Q³, Q⁴, Q⁵, Q⁶, Q⁷, R¹, R², R³ and R⁴ are as defined in Formula (41-I);
Q⁸ and Q⁹ are independently CH, N, NH, O, or S, provided at least one of Q⁸ and Q⁹ is N, NH, O, or S;
R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹⁰R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, or —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, or 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the SOS1 inhibitor is a compound having the structure of Formula (42-I),

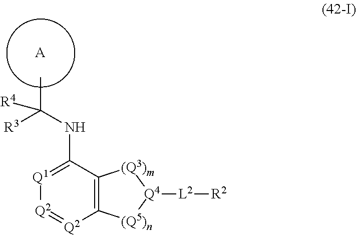

or a pharmaceutically acceptable salt, solvate, isomer, prodrug, or tautomer thereof, wherein:
Q¹ is CH orN;
Q⁴ is CH, C, or N;
each Q² is independently C—R¹ or N, wherein one Q² is N and the other Q² is C—R¹;
each Q³ and Q⁵ are independently C(R^{QC})₂, NR^{QN}, CO, O, S, or SO₂, wherein each R^{QC} is independently H, F, Cl, Br, or 6-10 membered aryl, and wherein each R^{QN} is independently H, C₁₋₆ alkyl, or 6-10 membered aryl;
wherein at least one of Q¹, Q², Q³, Q⁴, and Q⁵ is N, NR^{QN}, O, or SO₂;
m is 0, 1, 2, or 3;
n is 0, 1, 2, or 3;
wherein when m is 0, then n is not 0;
R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}; wherein R^{1a} is H, C₁₋₆ alkyl, cyclopropyl, 3-6 membered heterocyclyl, or C₁₋₆ haloalkyl;
L² is selected from the group consisting of a bond, —C(O)—, —C(O)O—, C(O)NH(CH₂)ₒ—, —SO₂,

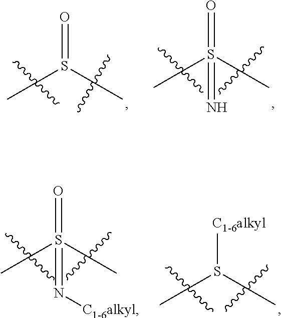

C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl.

Some embodiments of Formula (42-I) are characterized by the proviso that when

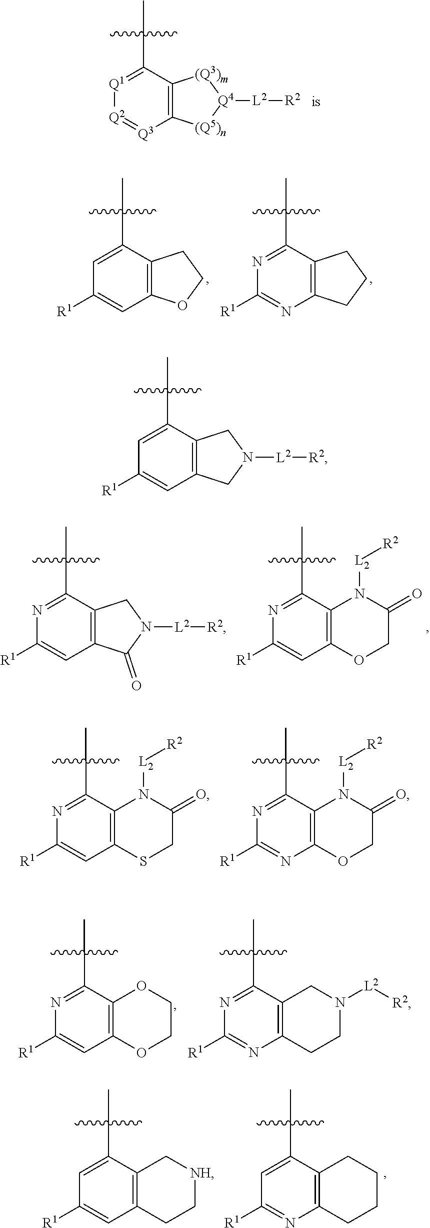

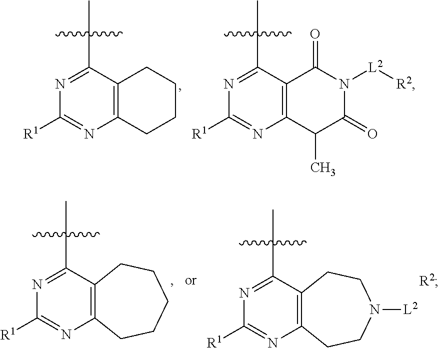

then R¹ is not H.

In some embodiments, the present invention is directed to a method of treating a subject having a disease or disorder characterized by SHP2-mediated activation of a RAS protein comprising administering to a subject in need of such treatment a therapeutically effective amount of a SOS1 inhibitor having the structure of Formula (42-I), or a pharmaceutically acceptable salt, solvate, hydrate, tautomer, or isomer thereof.

In some embodiments, the method of inhibiting SOS1 in a subject comprises administering to the subject in need of such treatment a therapeutically effective amount of a SOS1 inhibitor having the structure of Formula (42-I), or a pharmaceutically acceptable salt, solvate, hydrate, tautomer, or isomer thereof.

In some embodiments, the present invention is directed to a method of inhibiting the interaction of SOS1 and a RAS-family protein in a cell or inhibiting the interaction of SOS1 and RAC1 in a cell, comprising administering to the cell a SOS1 inhibitor having the structure of Formula (42-I), or a pharmaceutically acceptable salt, solvate, hydrate, tautomer, or isomer thereof.

In some embodiments, the present invention is directed to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof a therapeutically effective amount of a SOS1 inhibitor having the structure of Formula (42-I), or a pharmaceutically acceptable salt, solvate, hydrate, tautomer, or isomer thereof.

In some embodiments, the present invention is directed to a method of treating or preventing cancer in a subject in need thereof, comprising administering to the subject a therapeutically effective amount of a SOS1 inhibitor having the structure of Formula (42-I), or a pharmaceutically acceptable salt, solvate, hydrate, tautomer, or isomer thereof.

In some embodiments, the SOS1 inhibitor is a compound having the structure of Formula (42-I-a),

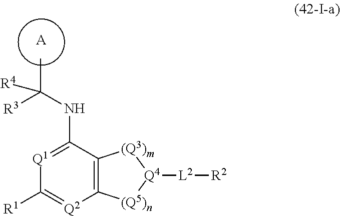

or a pharmaceutically acceptable salt, solvate, isomer, prodrug, or tautomer thereof, wherein:
Q¹, Q³, Q⁴, Q⁵, m, n and A are as defined in Formula (42-I);
Q² is CH or N;
wherein at least one of Q¹, Q², Q³, Q⁴, and Q⁵ is N, NR^{QN}, O, or SO₂;
R¹ is selected from the group consisting of H, halogen, C₁₋₆ alkyl, cyclopropyl, —CN, and —OR^{1a}; wherein R^{1a} is H or C₁₋₆ alkyl;
L² is selected from the group consisting of a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;
R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R², is H or C₁₋₆ alkyl; and
R³ and R⁴ are independently H or C₁₋₆ alkyl; wherein at least one of R³ and R⁴ is not H; or R³ and R⁴ together with the atom to which they are attached combine to form a 3-6 membered cycloalkyl.

In some embodiments, the SOS1 inhibitor is a compound having the structure of Formula (42-V),

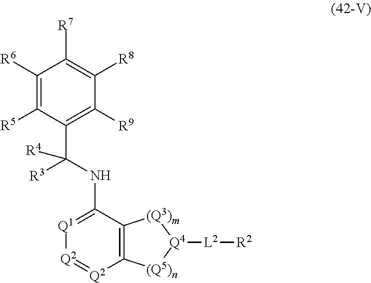

or a pharmaceutically acceptable salt, solvate, isomer, prodrug, or tautomer thereof, wherein:
L², Q¹, Q², Q³, Q⁴, Q⁵, m, n, R¹, R², R³ and R⁴ are as defined in Formula (42-I);
R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R, —NR¹⁰S(O)R¹¹, 3-8 membered cycloalkyl, 3-14 membered heterocyclyl, 6-10 membered aryl, or 5-10 membered heteroaryl, or any two adjacent R⁵, R⁶, R⁷, R⁸, and R⁹ forms a 3-14 membered fused ring;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the SOS1 inhibitor is a compound having the structure of Formula (42-VI),

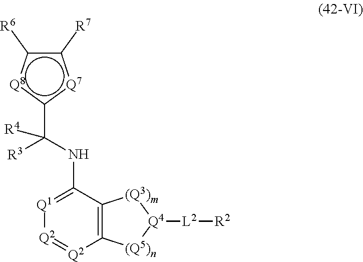

or a pharmaceutically acceptable salt, solvate, isomer, prodrug, or tautomer thereof, wherein:
L², Q¹, Q², Q³, Q⁴, Q⁵, m, n, R¹, R², R³, and R⁴ are as defined in Formula (42-I);
Q⁷ and Q⁸ are each independently CH, N, NH, O, or S, provided at least one of Q⁷ and Q⁸ is N, NH, O, or S;
R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl,
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the SOS1 inhibitor is a compound having the structure of Formula (48-I),

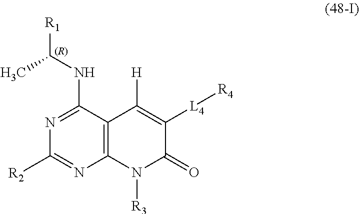

or a pharmaceutically acceptable salt, solvate, isomer, prodrug, or tautomer thereof, wherein:
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocyclyl, and further wherein R₂ₐ is selected from the group consisting of H, C₁₋₆ alkyl, 3-6 membered heterocyclyl, and C₁₋₆ haloalkyl;
R₃ is selected from the group consisting of H, C₁₋₃ alkyl, —OR₃ₐ, cyclopropyl, and 3-6 membered heterocyclyl, wherein each of C₁₋₃ alkyl, cyclopropyl, and 3-6 membered heterocyclyl is optionally substituted with R₃ₐ, and further wherein R₃ₐ is selected from the group consisting of C₁₋₃ alkyl, halogen, —OH, or —CN;
L₄ is selected from the group consisting of bond, —C(O)—, —C(O)O—, —C(O)NH(CH₂)ₒ—, —NH—, —S—, —S(O)₂—,

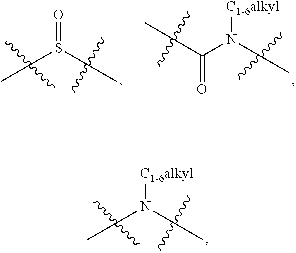

—(CH₂)ₚ—, and —O—; wherein o is 0, 1, or 2; and wherein p is a number from 1 to 6; and
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ;
   wherein R₄ₐ is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3;
   wherein each R_{4b} is independently H, C₁₋₆ alkyl; and
   wherein each R_{4c} is independently H or C₁₋₆ alkyl.

In some embodiments of compounds of Formula (48-I), R₁ is the optionally substituted 6-membered aryl. In some embodiments, the 6-membered aryl has the following structure:

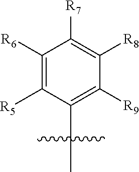

wherein R₅, R₆, R₇, R₈, and R₉ are as defined below in connection with Formula (48-II)-(48-IV).

In some embodiments of compounds of Formula (48-I), R₁ is the optionally substituted 5-6 membered heteroaryl. In some embodiments, R₁ is a 6-membered heteroaryl having any of the following structures:

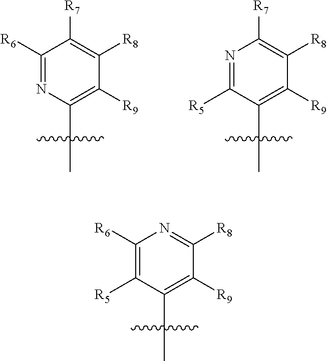

wherein R₅, R₆, R₇, R₈, and R₉ are as defined below in connection with Formula (48-II)-(48-IV).

In some embodiments of compounds of Formula (I), R₁ is the optionally substituted 5-6 membered heteroaryl. In some embodiments, R₁ is a 5-membered heteroaryl having the following structure:

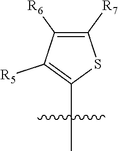

wherein R₅, R₆, and R₇ are as defined below in connection with Formula (48-II)-(48-IV).

In some embodiments, the present invention is directed to a method of treating a subject having a disease or disorder characterized by SHP2-mediated activation of a RAS protein comprising administering to a subject in need of such treatment a therapeutically effective amount of a SOS1 inhibitor having the structure of Formula (48-I), or a pharmaceutically acceptable salt, solvate, hydrate, tautomer, or isomer thereof.

In some embodiments, the method of inhibiting SOS1 in a subject comprises administering to the subject in need of such treatment a therapeutically effective amount of a SOS1 inhibitor having the structure of Formula (48-I), or a pharmaceutically acceptable salt, solvate, hydrate, tautomer, or isomer thereof.

In some embodiments, the present invention is directed to a method of inhibiting the interaction of SOS1 and a RAS-family protein in a cell or inhibiting the interaction of SOS1 and RAC1 in a cell, comprising administering to the cell a SOS1 inhibitor having the structure of Formula (48-I), or a pharmaceutically acceptable salt, solvate, hydrate, tautomer, or isomer thereof.

In some embodiments, the present invention is directed to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof a therapeutically effective amount of a SOS1 inhibitor having the structure of Formula (48-I), or a pharmaceutically acceptable salt, solvate, hydrate, tautomer, or isomer thereof.

In some embodiments, the present invention is directed to a method of treating or preventing cancer in a subject in need thereof, comprising administering to the subject a therapeutically effective amount of a SOS1 inhibitor having the structure of Formula (48-I), or a pharmaceutically acceptable salt, solvate, hydrate, tautomer, or isomer thereof.

In some embodiments, the SOS1 inhibitor is a compound having the structure of Formula (48-II),

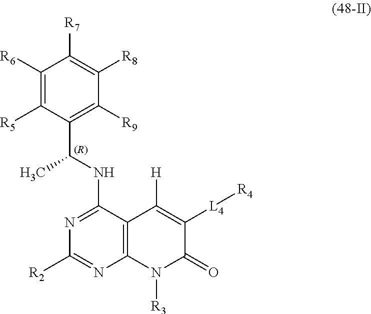

or a pharmaceutically acceptable salt, solvate, isomer, prodrug, or tautomer thereof, wherein R₂, R₃, L₄, and R₄ are as defined in Formula (48-I);
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl, 6-10 membered aryl, or 5-10 membered heteroaryl, or any two adjacent R⁵, R⁶, R⁷, R⁸, and R⁹ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂-s alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the SOS1 inhibitor is a compound having the structure of Formula (48-II),

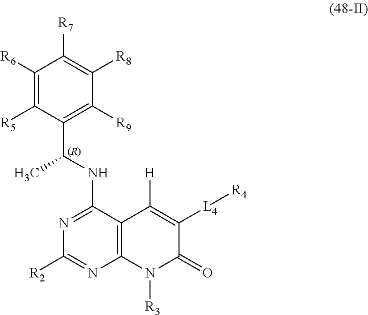

or a pharmaceutically acceptable salt or a stereoisomer thereof, wherein:
R₂ is H;
R₃ is selected from the group consisting of H and C₁₋₃ alkyl;
L₄ is a bond;
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ;
   wherein R₄ₐ is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3;
   wherein each R_{4b} is independently H, C₁₋₆ alkyl;
   wherein each R_{4c} is independently H or C₁₋₆ alkyl;
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, ═O, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl;
R₁₀, R₁₁, and R₁₂ are at each occurrence independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from the group consisting of H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the present disclosure relates to compounds having the structure of Formula (48-III),

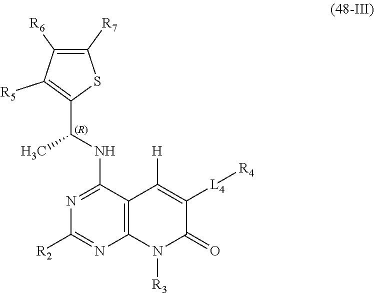

or a pharmaceutically acceptable salt, solvate, isomer, prodrug, or tautomer thereof, wherein R₂, R₃, L₄, and R₄ are as defined Formula (48-I);
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NRinR₁₂, —NR₁₀S(O)₂R₁₁, —S(O)NR₁₁R₁₂, —S(O)R₁₀, —NR₁₀S(O)NRinR₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl, or any two adjacent R⁵, R⁶, R⁷, R⁸, and R⁹ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the present disclosure relates to compounds having the structure of Formula (48-IV-a), (48-IV-b), or (48-IV-c),

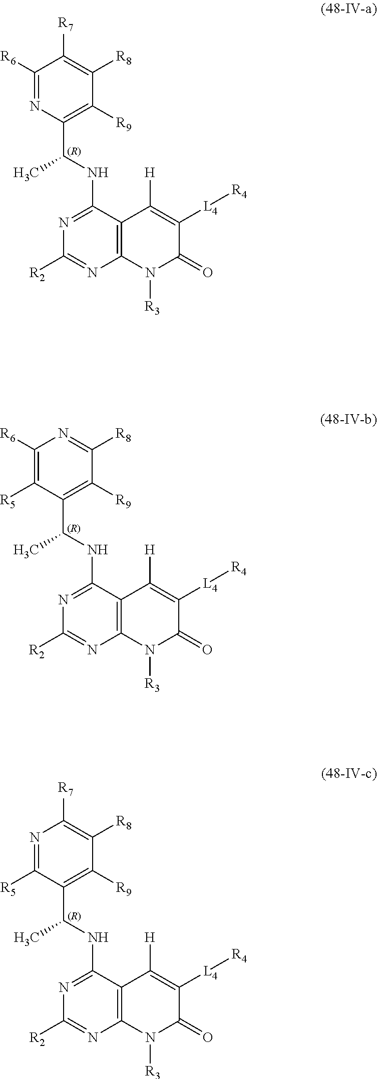

or a pharmaceutically acceptable salt, solvate, isomer, prodrug, or tautomer thereof, wherein R₂, R₃, L₄, and R₄ are as defined Formula (48-I);
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NRinR₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl, or any two adjacent R⁵, R⁶, R⁷, R⁸, and R⁹ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the present disclosure relates to compounds having the structure of Formula (48-IV-a) or Formula (48-IV-b).

In some embodiments of compounds of Formula (48-II)-(48-IV), one to three of R₅, R₆, R₇, R₈, and R₉ is C₁₋₆ alkyl, wherein the alkyl is optionally substituted with halogen.

In some embodiments of compounds of Formula (48-II)-(48-IV), one to three of R₅, R₆, R₇, R₈, and R₉ is C₁₋₆ alkyl, wherein the alkyl is optionally substituted with halogen or —OH.

In some embodiments of compounds of Formula (48-II)-(48-IV), one to three of R₅, R₆, R₇, R₈, and R₉ is C₁₋₆ alkyl, and one to three of R₅, R₆, R₇, R₈, and R₉ is C₁₋₆ alkyl optionally substituted with halogen.

In some embodiments of compounds of Formula (48-II)-(48-IV), one to three of R₅, R₆, R₇, R₈, and R₉ is halogen, and one to three of R₅, R₆, R₇, R₈, and R₉ is C₁₋₆ alkyl optionally substituted with halogen.

In some embodiments of compounds of Formula (48-II)-(48-IV), one to three of R₅, R₆, R₇, R₈, and R₉ is —NH₂.

In some embodiments of compounds of Formula (48-II)-(48-IV), one of R₅, R₆, R₇, R₈, and R₉ is —NH₂; and one of R₅, R₆, R₇, R₈, and R₉ is C₁₋₆ alkyl optionally substituted with halogen.

In some embodiments of compounds of Formula (48-II)-(48-IV), any two adjacent R⁵, R⁶, R⁷, R⁸, and R⁹ forms a 3-14 membered fused ring. In some embodiments of compounds of Formula (48-II)-(48-IV), any two adjacent R⁵, R⁶, R⁷, R⁸, and R⁹ forms a 3-8 membered fused ring. In some embodiments of compounds of Formula (48-II)-(48-IV), any two adjacent R⁵, R⁶, R⁷, R⁸, and R⁹ forms a 4-8 membered fused ring. In some embodiments of compounds of Formula (48-II)-(48-IV), any two adjacent R⁵, R⁶, R⁷, R⁸, and R⁹ forms a 4-membered fused ring or a 5-membered fused ring. In some embodiments, the fused ring is a 3-8 membered heterocyclyl or a 3-8 membered cycloalkyl. In some embodiments, the fused ring is a 4-8 membered heterocyclyl or a 4-8 membered cycloalkyl. In some embodiments, the fused ring is a 4-membered heterocyclyl or a 5-membered heterocyclyl. In some embodiments, the fused ring is a 4-membered cycloalkyl or a 5-membered cycloalkyl. In some embodiments, the fused ring is optionally substituted with —OH, C₁₋₆ alkyl, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl, 6-10 membered aryl, or 5-10 membered heteroaryl. In some embodiments, the fused ring is optionally substituted with halogen.

In some embodiments of compounds of Formula (48-II)-(48-IV), one or more of R₅, R₆, R₇, R₈, and R₉ is selected from among —CF₃, —NH₂, —F, and —CF₂CH₂OH.

In some embodiments of compounds of Formula (II), one of R₅, R₆, R₇, R₈, and R₉ is-CF₃ and one of R₅, R₆, R₇, R₈, and R₉ is —NH₂. In some embodiments of compounds of Formula (48-II), one of R₅, R₆, R₇, R₈, and R₉ is —F and one of R₅, R₆, R₇, R₈, and R₉ is —CF₂CH₂OH.

In some embodiments of compounds of Formula (48-I), R₁ is selected from among:

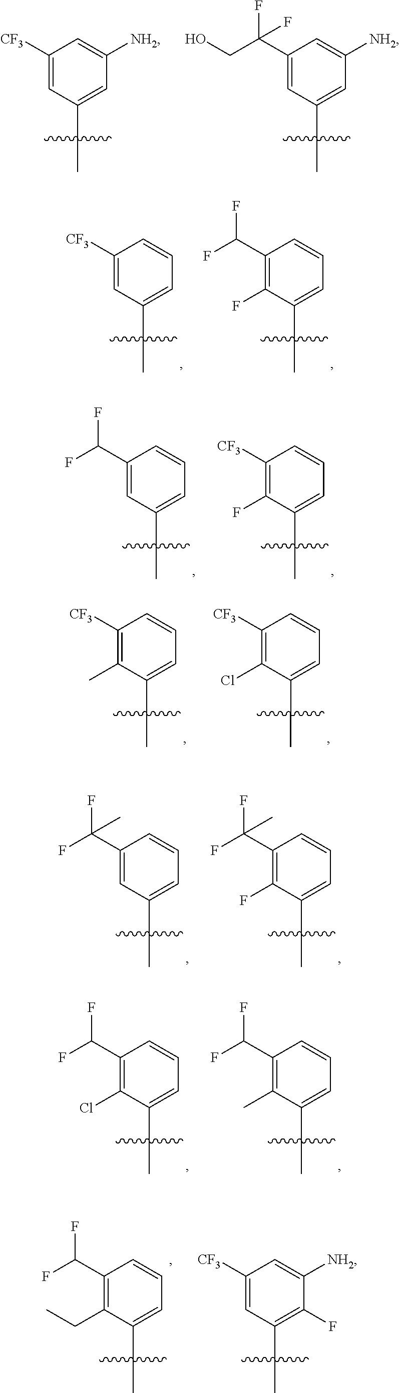

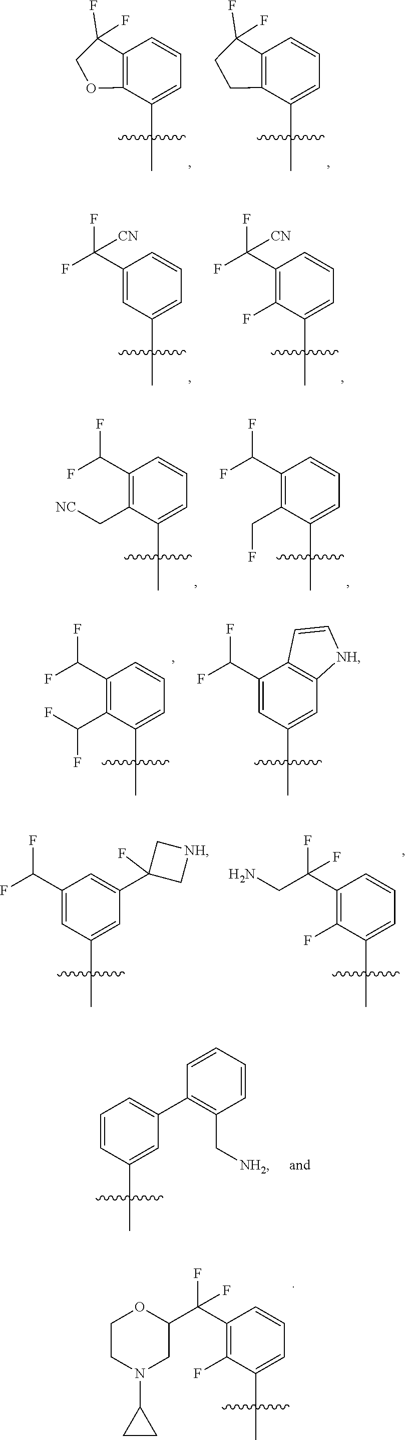

In some embodiments of compounds of Formula (48-I), R₁ is selected from among:

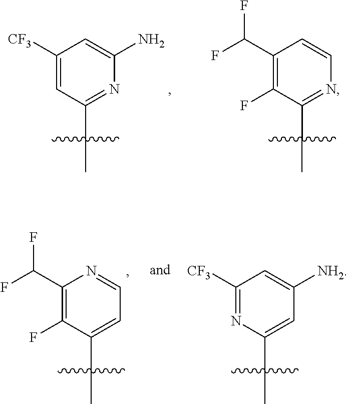 [SMILES: *c1cc(C(F)(F)F)cc(N)n1 ; *c1nccc(C(F)F)c1F ; *c1ccnc(C(F)F)c1F ; *c1cc(N)cc(C(F)(F)F)n1]

In some embodiments of compounds of Formula (48-I), R₁ is selected from among:

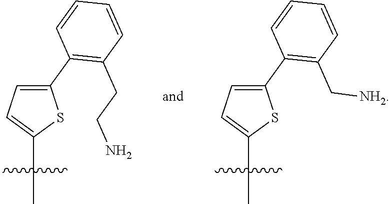 [SMILES: *c1ccc(-c2ccccc2CCN)s1 ; *c1ccc(-c2ccccc2CN)s1]

In some embodiments of compounds of Formula (48-I), R₁ is selected from among:

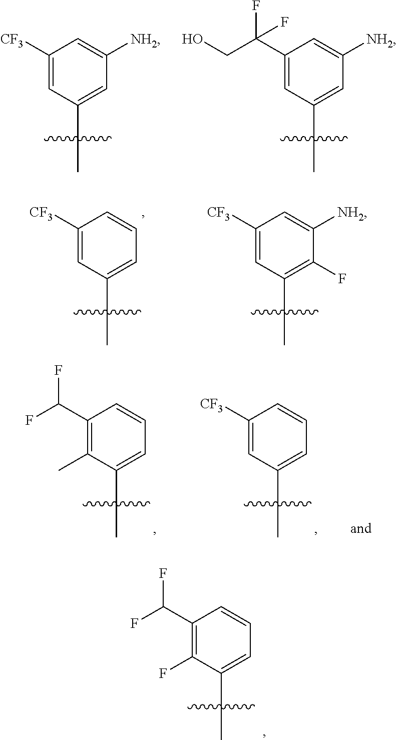

In some embodiments of compounds of Formula (48-I)-(48-IV), R₂ is H.

In some embodiments of compounds of Formula (48-I)-(48-IV), R₂ is C₁₋₆ alkyl. In some embodiments of compounds of Formula (I)-(IV), R₂ is —CH₃.

In some embodiments of compounds of Formula (48-I)-(48-IV), R₂ is C₁₋₆ alkyl substituted with 5-6 membered heterocycloalkyl. In some embodiments of compounds of Formula (48-I)-(48-IV), R₂ is

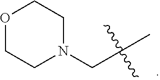 [SMILES: *CN1CCOCC1]

In some embodiments of compounds of Formula (48-I)-(48-IV), R₂ is C₁₋₆ alkyl substituted with —NHR₂ₐ, wherein R₂ₐ is C₁₋₆ alkyl or 3-6 membered heterocyclyl. In some embodiments of compounds of Formula (48-I)-(48-IV), R₂ is selected from among

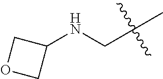 [SMILES: *CNC1COC1]

and —CH₂NHCH₃.

In some embodiments of compounds of Formula (48-I)-(48-IV), R₂ is C₁₋₆ alkyl substituted with —OR₂ₐ, wherein R₂ₐ is H or C₁₋₆ alkyl. In some embodiments of compounds of Formula (48-I)-(48-IV), R₂ is —CH₂OH.

In some embodiments of compounds of Formula (48-I)-(48-IV), R₂ is —NHR₂ₐ, wherein R₂ₐ is C₁₋₆ alkyl. In some embodiments of compounds of Formula (48-I)-(48-IV), R₂ is —NHCH₃.

In some embodiments of compounds of Formula (48-I)-(48-IV), R₂ is —OR₂ₐ; wherein R₂ₐ is C₁₋₆ alkyl. In some embodiments of compounds of Formula (48-I)-(48-IV), R₂ is —OCH₃.

In some embodiments of compounds of Formula (48-I)-(48-IV), R₃ is C₁₋₃ alkyl. In some embodiments of compounds of Formula (48-I)-(48-IV), R₃ is —CH₃. In some embodiments of compounds of Formula (48-I)-(48-IV), R₃ is —CD₃.

In some embodiments of compounds of Formula (48-I)-(48-IV), R₃ is C₁₋₃ alkyl substituted with —OH. In some embodiments of compounds of Formula (I)-(IV), R₃ is —CH₂CH₂OH.

In some embodiments of compounds of Formula (48-I)-(48-IV), R₃ is H.

In some embodiments of compounds of Formula (48-I)-(48-IV), R₃ is —OR₃ₐ. In some embodiments of compounds of Formula (I)-(IV), R₃ is —OCH₃.

In some embodiments of compounds of Formula (48-I)-(48-IV), R₃ is cyclopropyl.

In some embodiments of compounds of Formula (48-I)-(48-IV), R₃ is 3-6 membered heterocyclyl. In some embodiments of compounds of Formula (48-I)-(48-IV), R₃ is

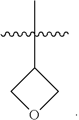 [SMILES: *C1COC1]

In some embodiments of compounds of Formula (48-I)-(48-IV), L₄ is selected from the group consisting of bond, —C(O)—, —C(O)O—, —C(O)NH(CH₂)ₒ-, —NH—, —S—, —S(O)₂—,

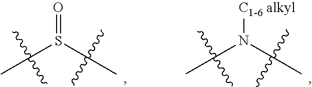

—(CH₂)ₚ—, and —O—; wherein o is 0, 1, or 2; and wherein p is a number from 1 to 6.

In some embodiments of compounds of Formula (48-I)-(48-IV), L₄ is a bond.

In some embodiments of compounds of Formula (48-I)-(48-IV), L₄ is —C(O)—.

In some embodiments of compounds of Formula (48-I)-(48-IV), L₄ is —(CH₂)ₚ—. In some embodiments of compounds of Formula (I), L₄ is —(CH₂)—.

In some embodiments of compounds of Formula (48-I)-(48-IV), R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl, 3-7 membered heterocyclyl, 6-10 membered aryl, or 5-10 membered heteroaryl.

In some embodiments of compounds of Formula (48-I)-(48-IV), R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl —OR₄ₐ, ═O, halogen, —C(O)R₄ₐ, —C(OO)R₄ₐ, —C(O)NR_{4b}R_{4c}, —CN, —NR_{4b}R_{4c},3-6 membered cycloalkyl, 3-7 membered heterocyclyl, 6-10 membered aryl, or 5-10 membered heteroaryl.

In some embodiments of compounds of Formula (48-I)-(48-IV), R₄ₐ is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3; wherein each R_{4b} is independently H, C₁₋₆ alkyl; and wherein each R₄ₖ is independently H or C₁₋₆ alkyl.

In some embodiments of compounds of Formula (48-I)-(48-IV), R₄ₐ is H, C₁₋₆ alkyl, C₁₋₆ haloalkyl, —C(O)R_{4b}, —C(O)NR_{4b}R_{4c}, 3-6 membered cycloalkyl, 6-10 membered aryl optionally substituted with —OR_{4b}, —CN, 3-7 membered heterocyclyl, —(CH₂)ᵣOCH₃, or —(CH₂)ᵣOH, wherein r is 1, 2, or 3; wherein each R_{4b} is independently H, C₁₋₆ alkyl; and wherein each R₄, is independently H or C₁₋₆ alkyl.

In some embodiments of compounds of Formula (48-I)-(48-IV), R₄ is 3-14 membered heterocyclyl. In some embodiments of compounds of Formula (48-I)-(48-IV), R₄ is substituted 3-14 membered heterocyclyl.

In some embodiments of compounds of Formula (48-I)-(48-IV), R₄ is 3-14 membered heterocyclyl substituted with 3-6 membered heterocyclyl. In some embodiments, the heterocyclyl substituent is oxetanyl.

In some embodiments of compounds of Formula (48-I)-(48-IV), R₄ is 3-14 membered heterocyclyl substituted with C₁₋₆ alkyl. In some embodiments of compounds of Formula (48-I)-(48-IV), R₄ is 3-14 membered heterocyclyl substituted with —CH₃. In some embodiments of compounds of Formula (48-I)-(48-IV), R₄ is 3-14 membered heterocyclyl substituted with —CH₂—, i.e., the substituent is a methylene bridge bridging 2 carbon atoms in the heterocyclyl ring.

In some embodiments of compounds of Formula (48-I)-(48-IV), R₄ is 3-14 membered heterocyclyl substituted with 3-6 membered cycloalkyl. In some embodiments, the cycloalkyl substituent is cyclopropyl.

In some embodiments of compounds of Formula (48-I)-(48-IV), R₄ is 3-14 membered heterocyclyl substituted with ═O.

In some embodiments, the R₄ is a heterocyclyl selected from among:

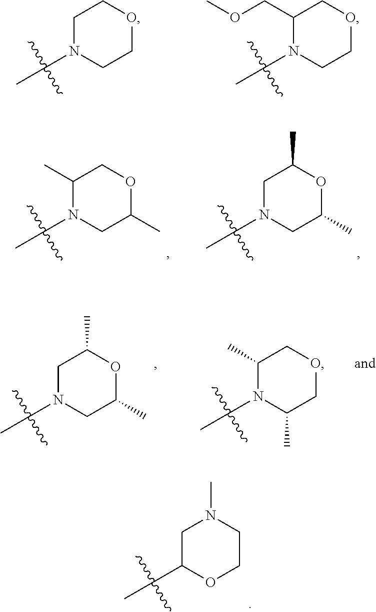

In some embodiments, the R₄ is a heterocyclyl selected from among:

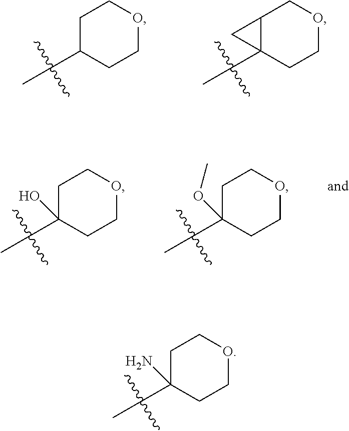

In some embodiments, the R₄ is a heterocyclyl selected from among:

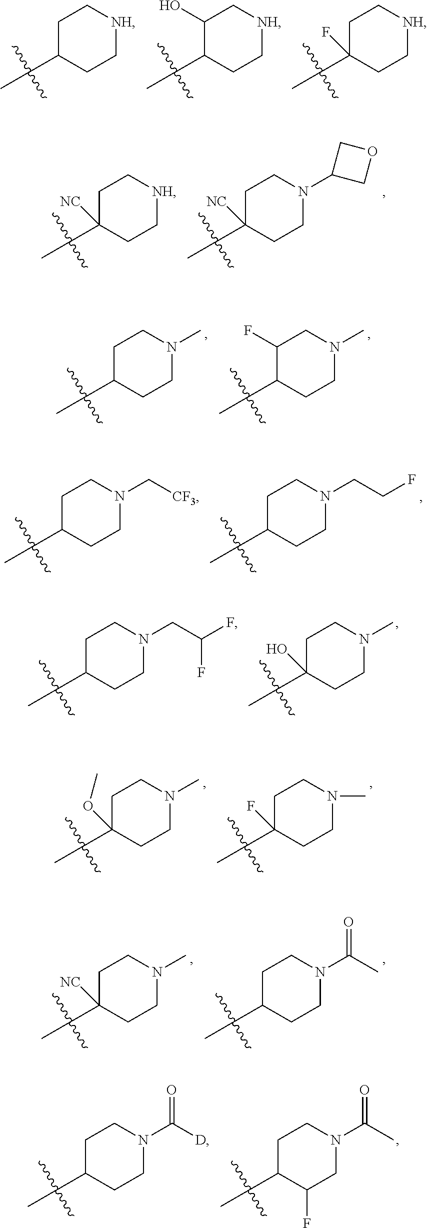

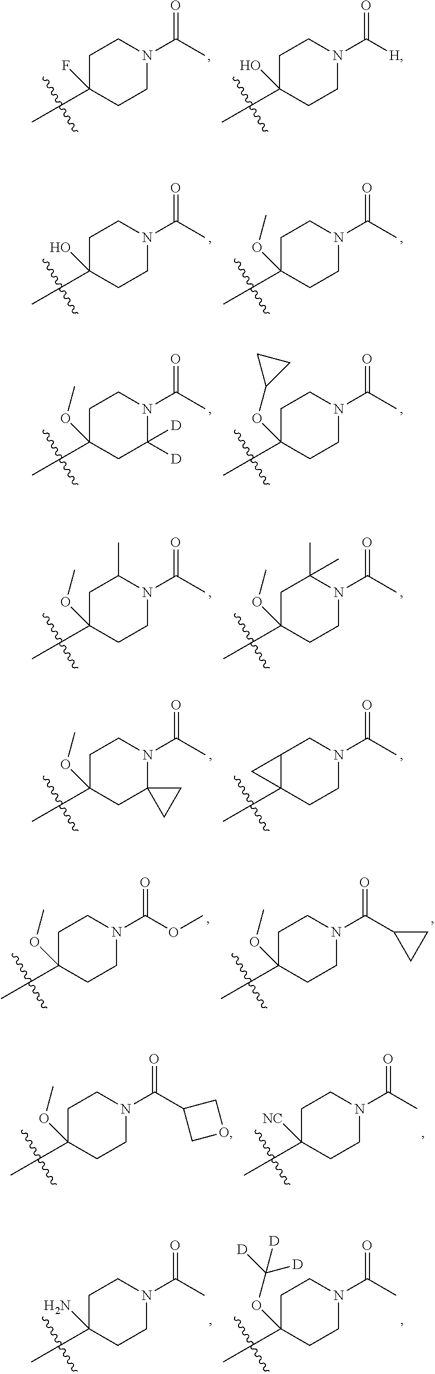

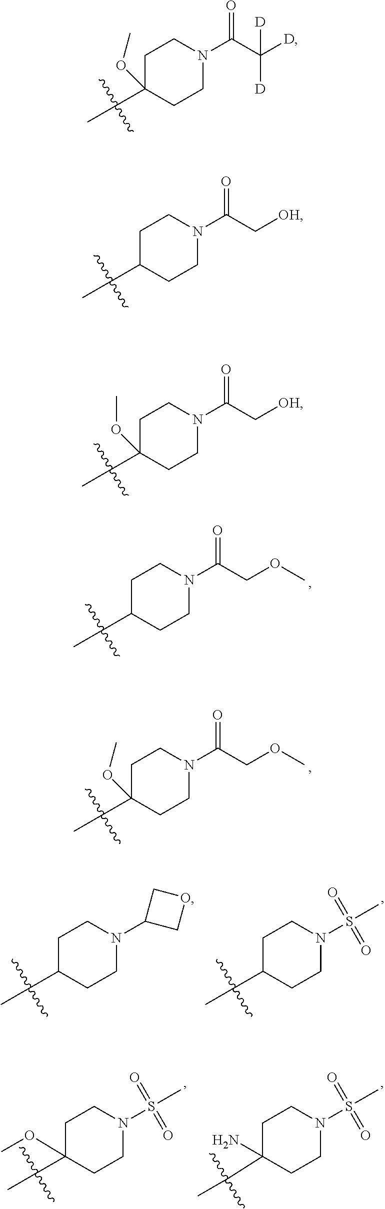

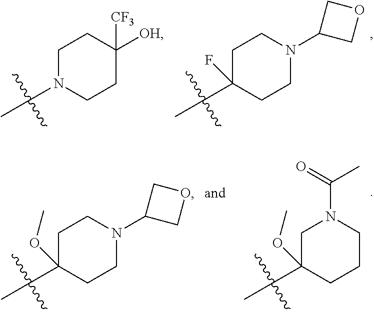 [SMILES: *N1CCC(O)(C(F)(F)F)CC1 ; *C1(F)CCN(C2COC2)CC1 ; *C1(OC)CCN(C2COC2)CC1 ; "and" ; *C1(OC)CCCN(C(C)=O)C1]

In some embodiments, the R₄ is a heterocyclyl selected from among:

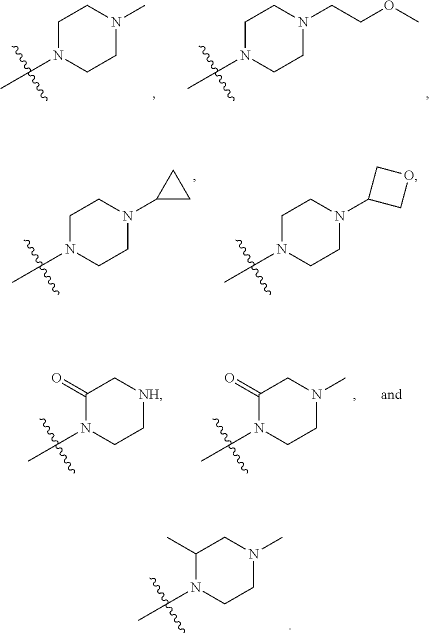

In some embodiments, the R₄ is a heterocyclyl selected from among:

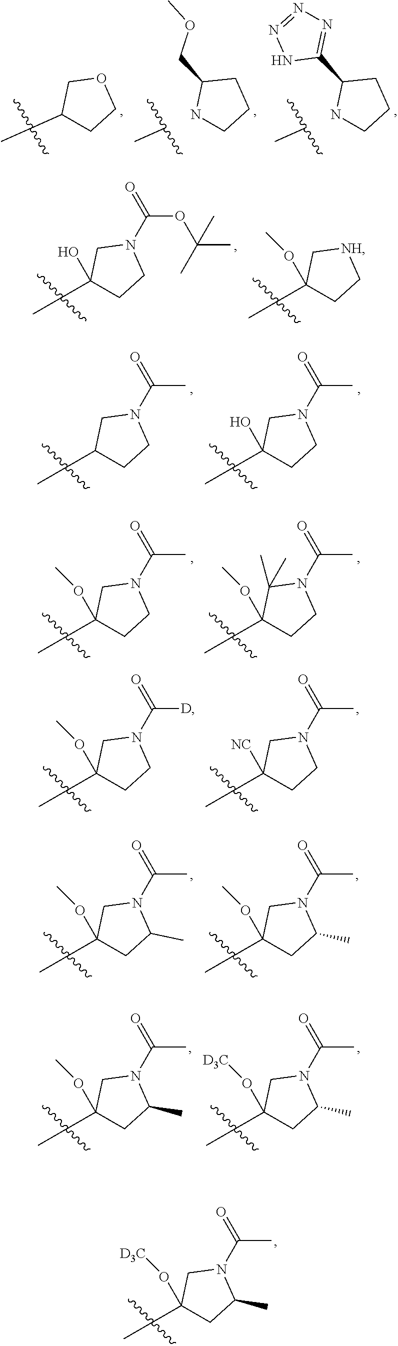

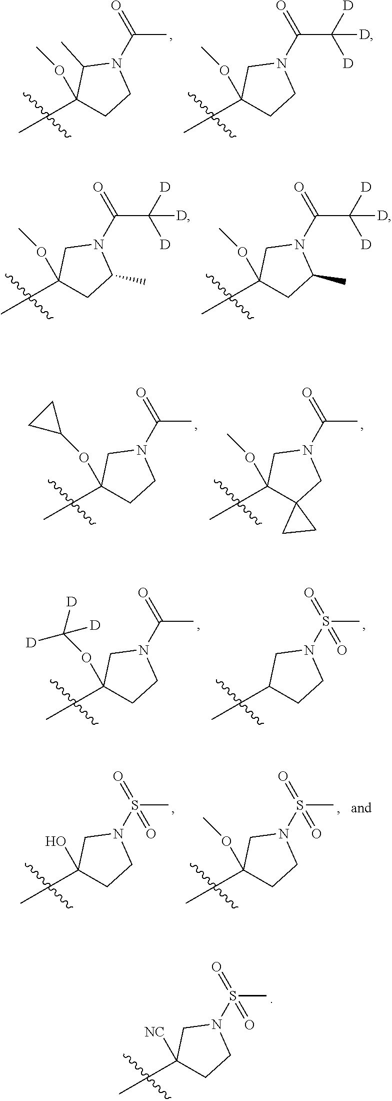

In some embodiments, the R₄ is a heterocyclyl selected from among:

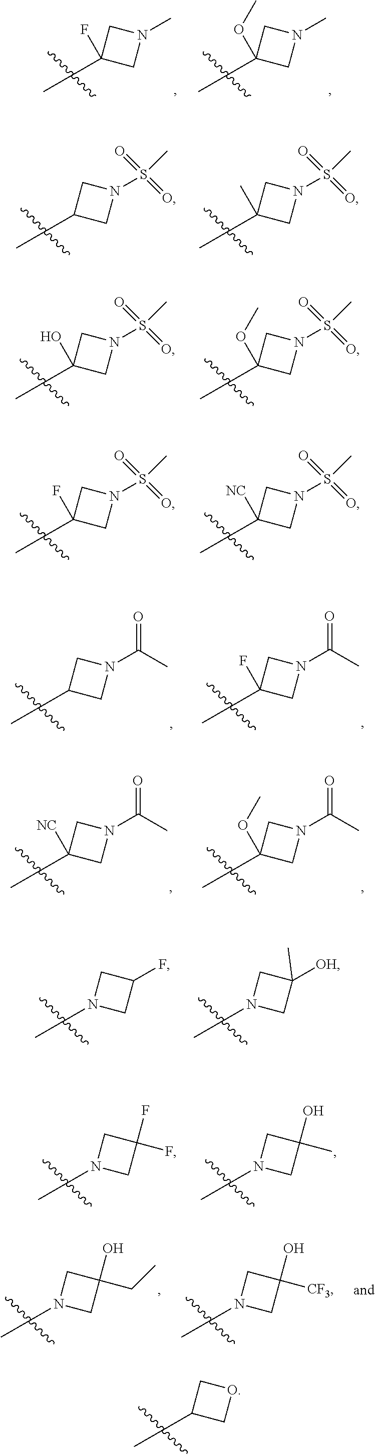

In some embodiments, the R₄ is a heterocyclyl selected from among:

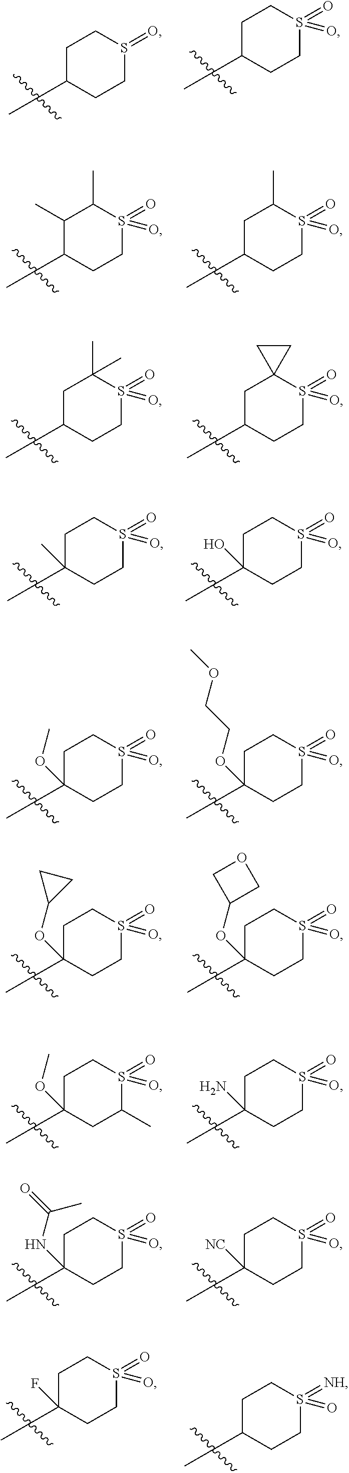

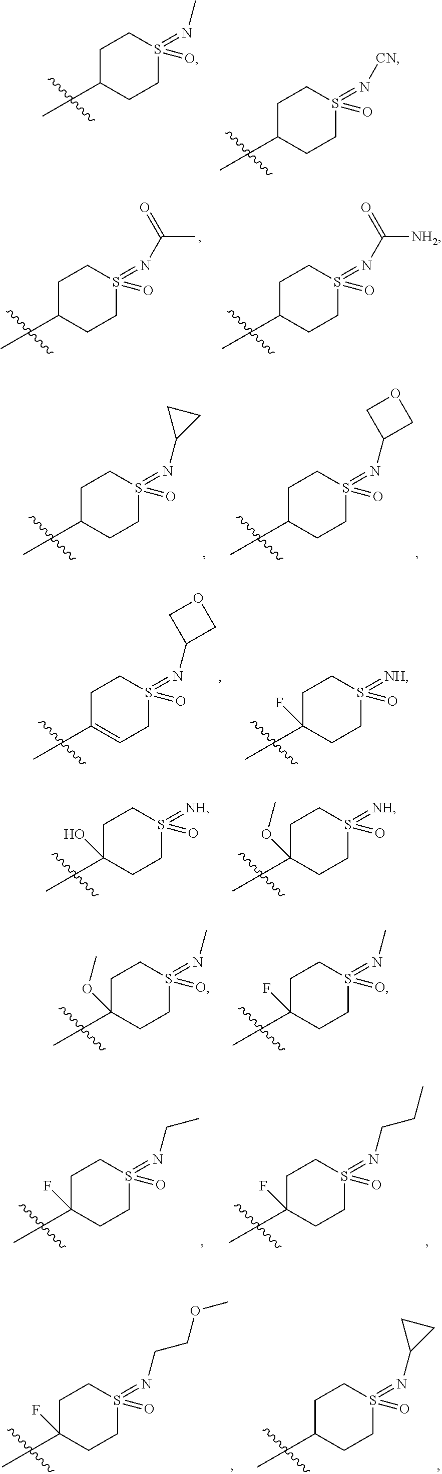

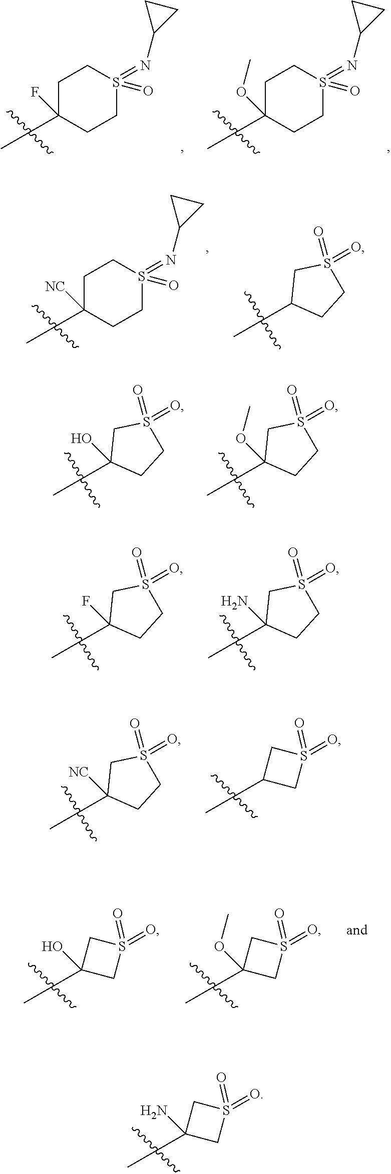

In some embodiments, the R₄ is a heterocyclyl selected from among:

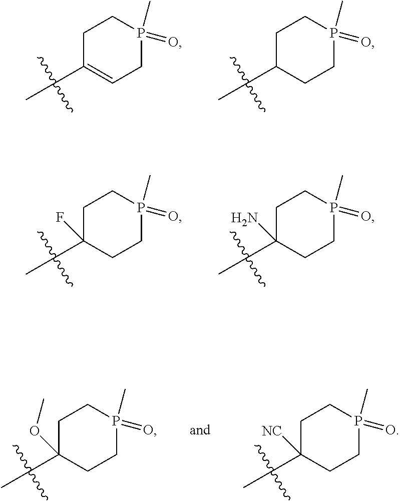

In some embodiments, the R₄ is a heterocyclyl selected from among:

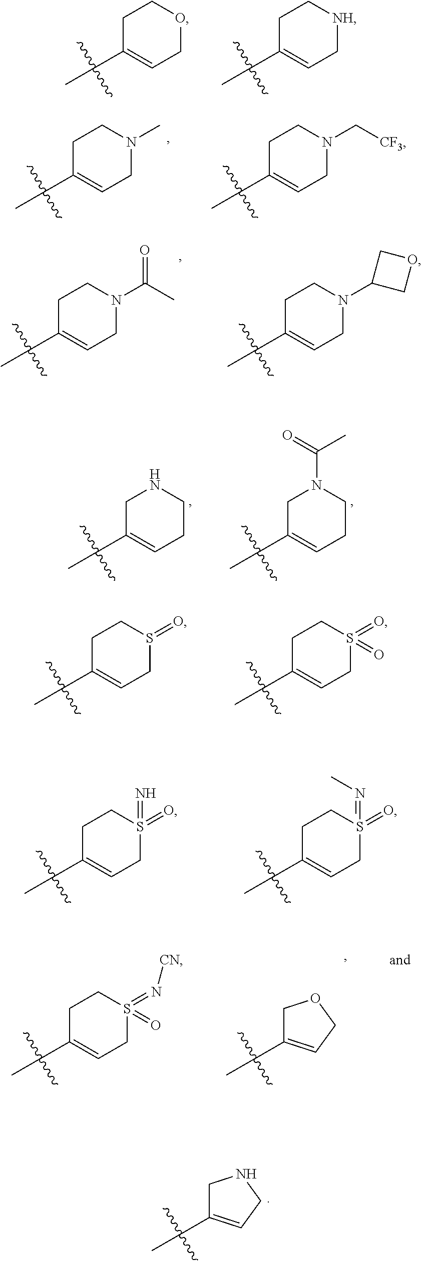

In some embodiments, the R₄ is a heterocyclyl selected from among:

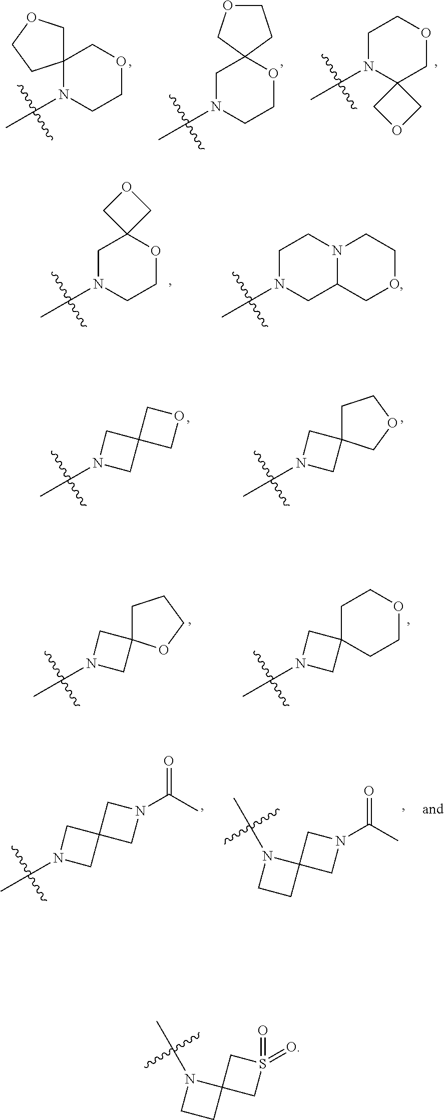

In some embodiments, the R₄ is a heterocyclyl selected from among:

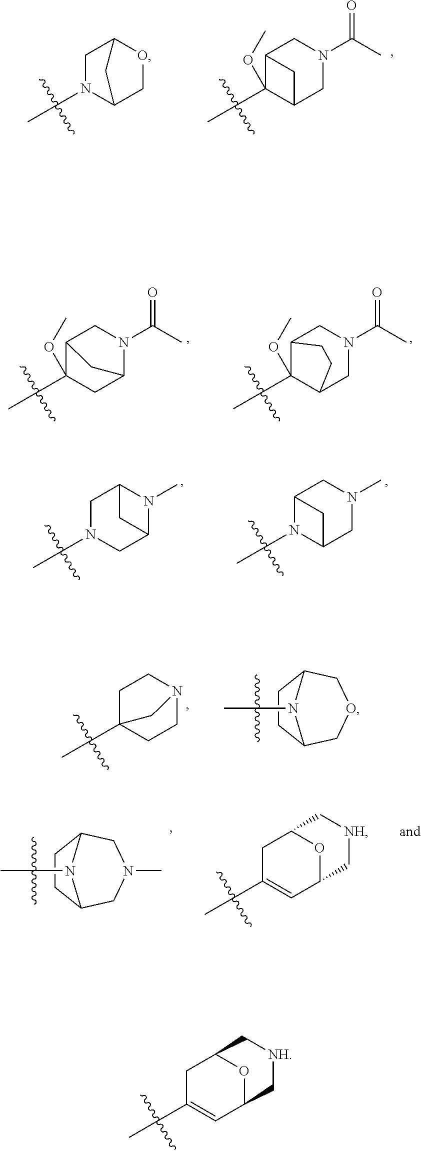

In some embodiments, the R₄ is a heterocyclyl selected from among:

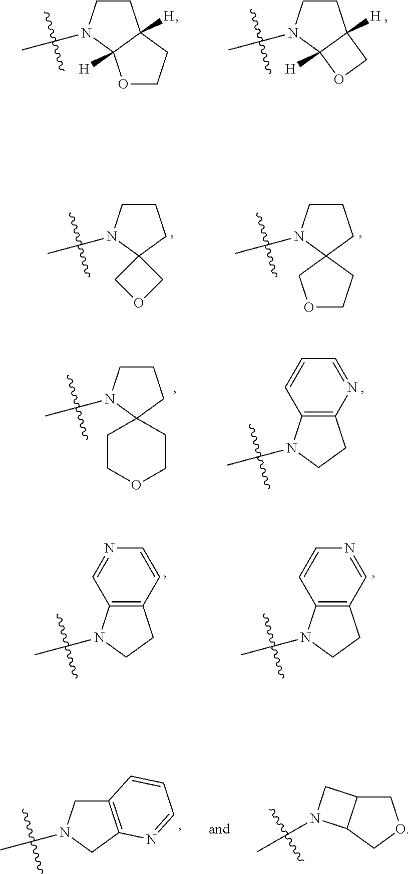

In some embodiments, the R₄ is a heterocyclyl selected from among:

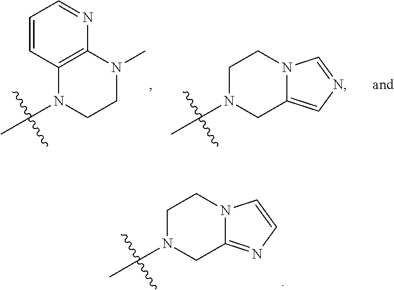 [SMILES: *N1CCN(C)c2ncccc21 ; *N1CCn2cncc2C1 ; *N1CCn2ccnc2C1]

In some embodiments, the R₄ is a heterocyclyl selected from among:

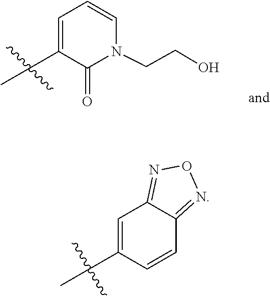 [SMILES: *c1cccn(CCO)c1=O ; *c1ccc2nonc2c1]

In some embodiments, the R₄ is a heterocyclyl selected from among:

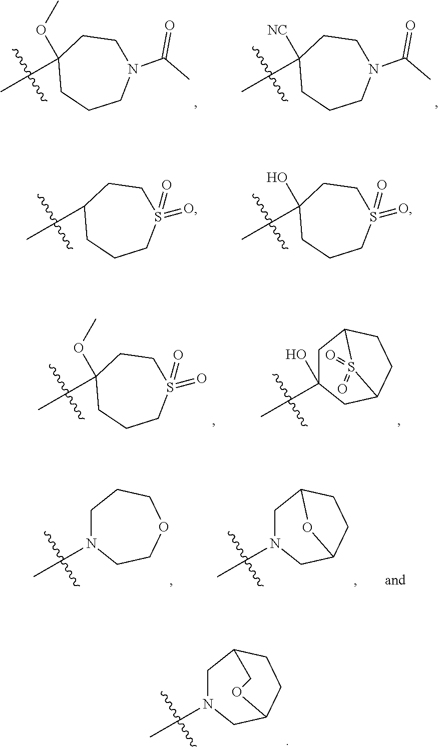

In some embodiments, R₄ is selected from among:

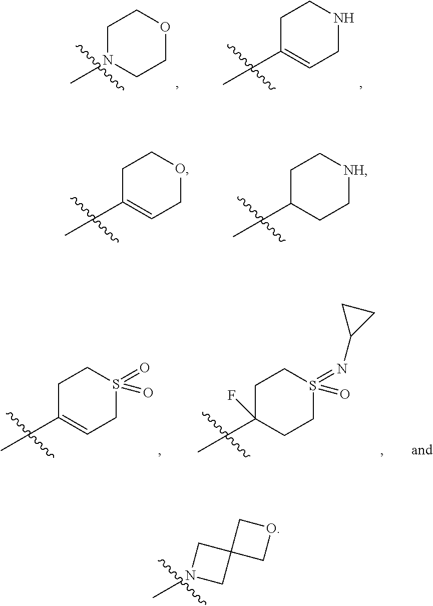

In some embodiments, R₄ is 3-14 membered cycloalkyl. In some embodiments, R₄ is substituted 3-14 membered cycloalkyl.

In some embodiments, R₄ is selected from among:

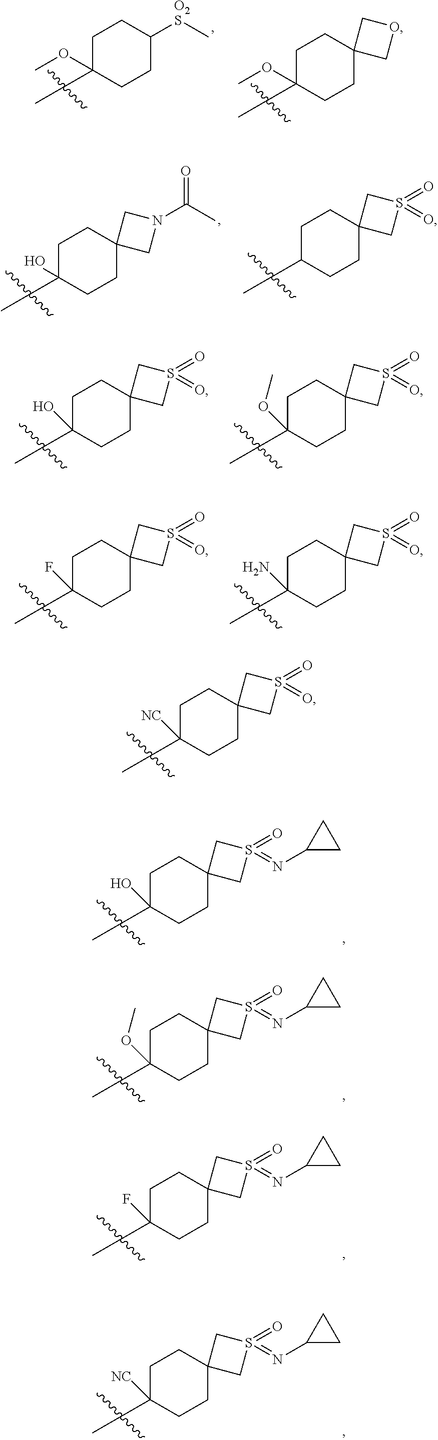

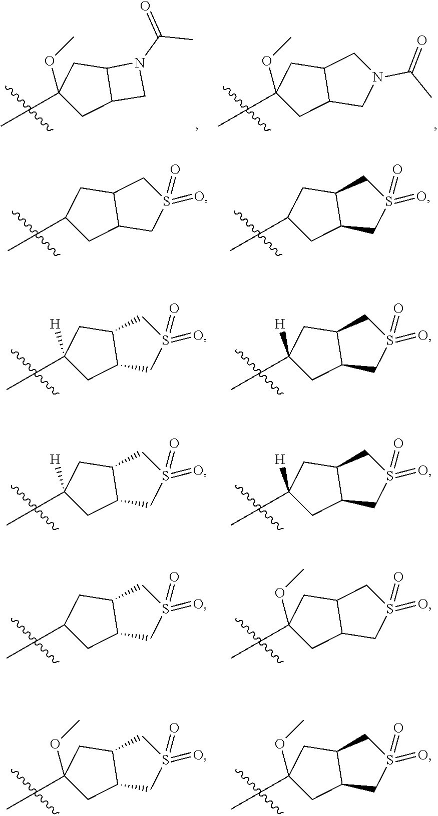

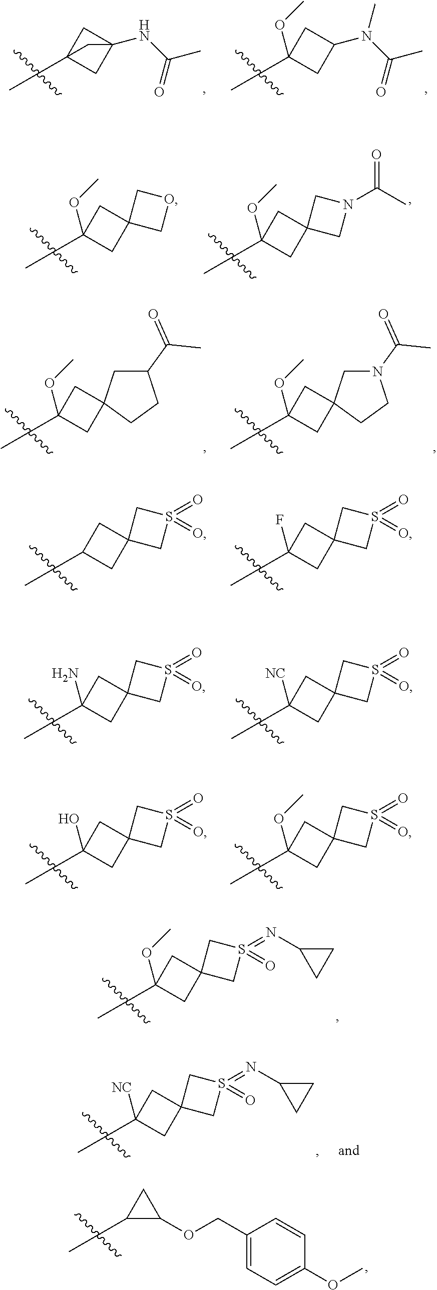

In some embodiments, R₄ is 6-10 membered aryl. In some embodiments, R₄ is substituted 6-10 membered aryl. In some embodiments, R₄ is phenyl. In some embodiments, R₄ is phenyl substituted with one or two group selected from among —OCH₃ and —CN.

In some embodiments, R₄ is 5-10 membered heteroaryl. In some embodiments, R₄ is substituted 5-10 membered heteroaryl. In some embodiments, R₄ is selected from among 1H-pyrrole, thiazole, pyridine, pyridazine, pyrimidine, each of which is optionally substituted with a group selected from among —F, —OCH₃, and —OCH₂CH₂OH.

In some embodiments, R₄ is selected from among:

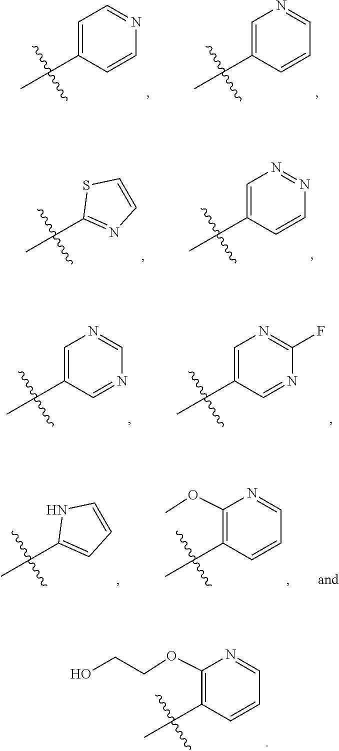

In some embodiments, the SOS1 inhibitor is a compound selected from the group consisting of the compounds in the following table, or a pharmaceutically acceptable salt, solvate, isomer, prodrug, or tautomer thereof:

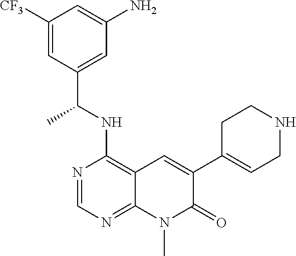 [SMILES: C[C@@H](Nc1ncnc2c1cc(C1=CCNCC1)c(=O)n2C)c1cc(N)cc(C(F)(F)F)c1]

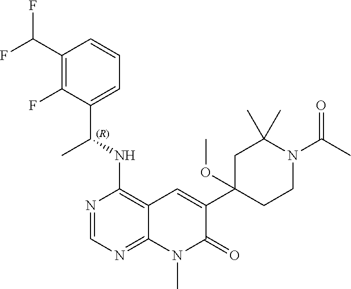

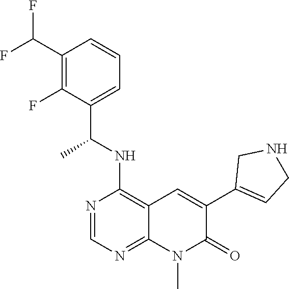 [SMILES: C[C@@H](Nc1ncnc2c1cc(C1=CCNC1)c(=O)n2C)c1cccc(C(F)F)c1F]

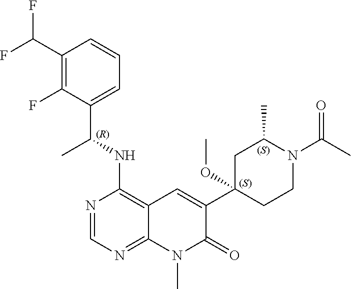

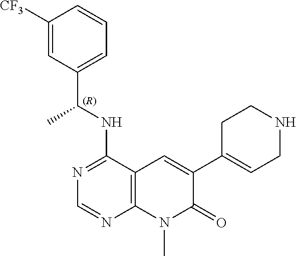

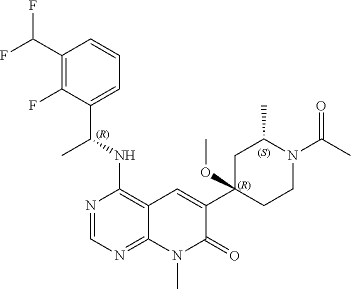

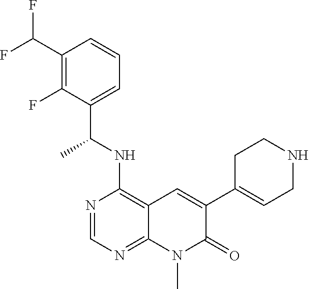 [SMILES: C[C@@H](Nc1ncnc2c1cc(C1=CCNCC1)c(=O)n2C)c1cccc(C(F)F)c1F]

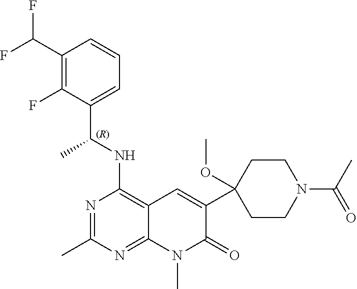

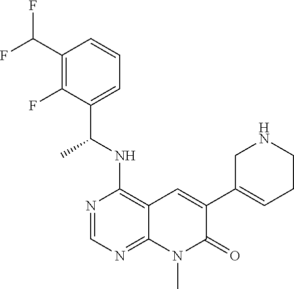 [SMILES: C[C@@H](Nc1ncnc2c1cc(C1=CCCNC1)c(=O)n2C)c1cccc(C(F)F)c1F]

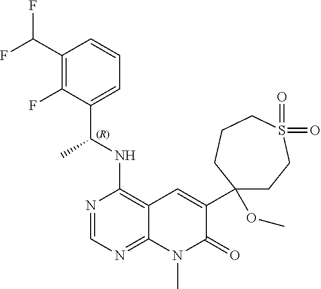

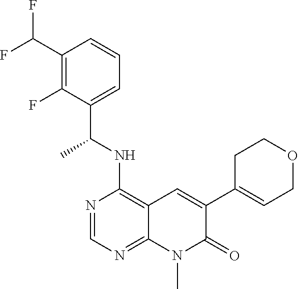 [SMILES: C[C@@H](Nc1ncnc2c1cc(C1=CCOCC1)c(=O)n2C)c1cccc(C(F)F)c1F]

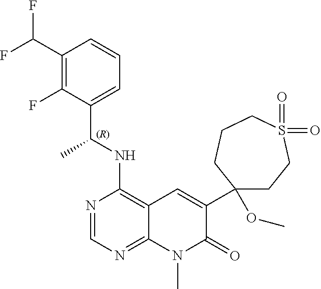

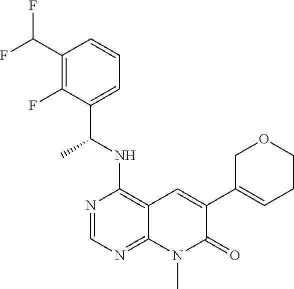 [SMILES: C[C@@H](Nc1ncnc2c1cc(C1=CCCOC1)c(=O)n2C)c1cccc(C(F)F)c1F]

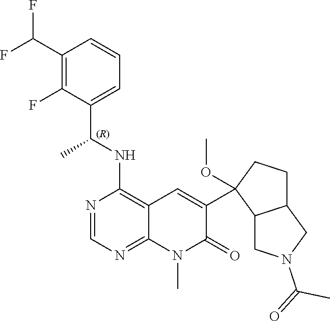

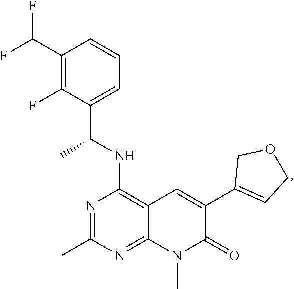 [SMILES: Cc1nc(N[C@H](C)c2cccc(C(F)F)c2F)c2cc(C3=CCOC3)c(=O)n(C)c2n1]

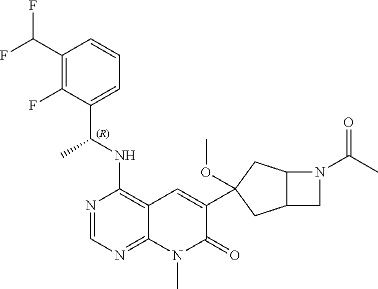

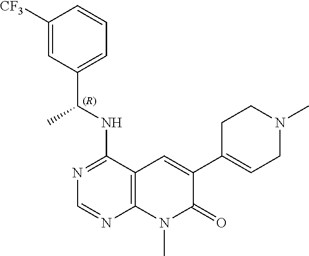

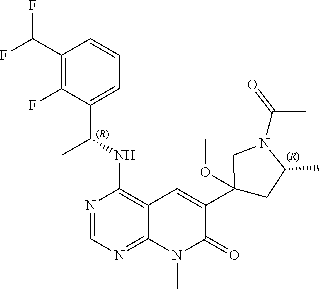

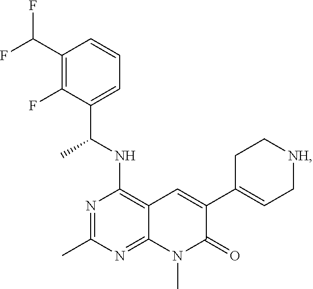 [SMILES: Cc1nc(N[C@H](C)c2cccc(C(F)F)c2F)c2cc(C3=CCNCC3)c(=O)n(C)c2n1]

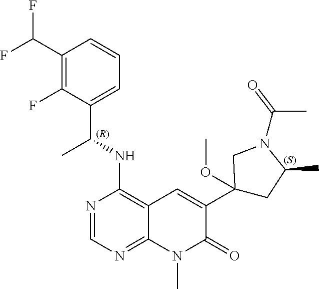

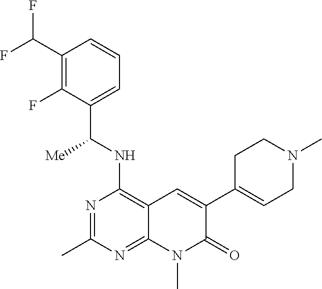 [SMILES: Cc1nc(N[C@H](C)c2cccc(C(F)F)c2F)c2cc(C3=CCN(C)CC3)c(=O)n(C)c2n1]

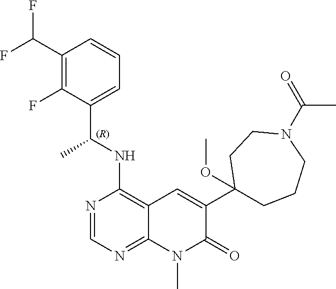

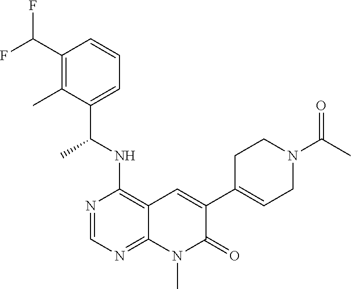 [SMILES: CC(=O)N1CC=C(c2cc3c(N[C@H](C)c4cccc(C(F)F)c4C)ncnc3n(C)c2=O)CC1]

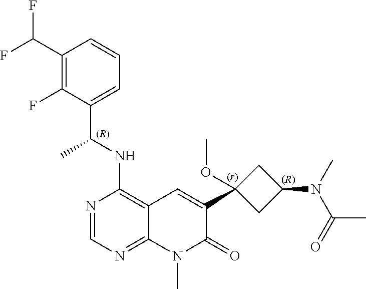

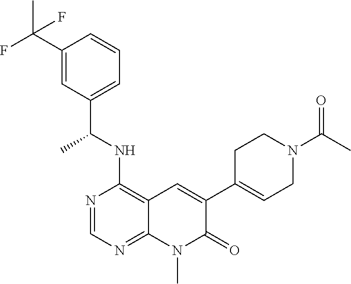 [SMILES: CC(=O)N1CC=C(c2cc3c(N[C@H](C)c4cccc(C(C)(F)F)c4)ncnc3n(C)c2=O)CC1]

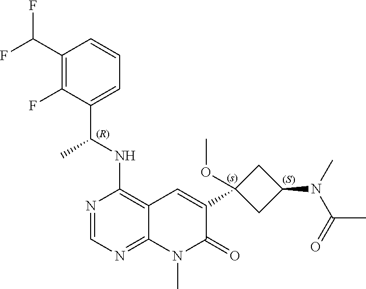

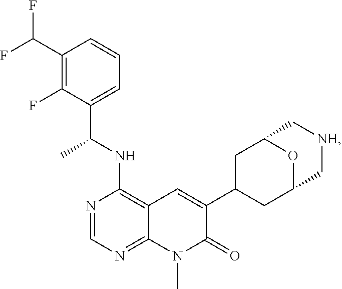 [SMILES: C[C@@H](Nc1ncnc2c1cc(C1C[C@H]3CNC[C@@H](C1)O3)c(=O)n2C)c1cccc(C(F)F)c1F]

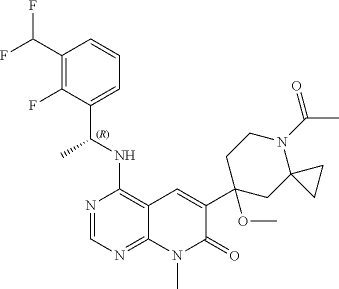

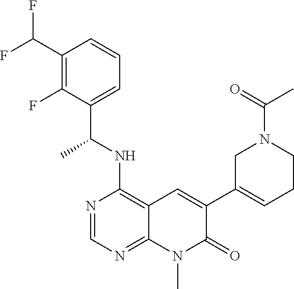 [SMILES: CC(=O)N1CCC=C(c2cc3c(N[C@H](C)c4cccc(C(F)F)c4F)ncnc3n(C)c2=O)C1]

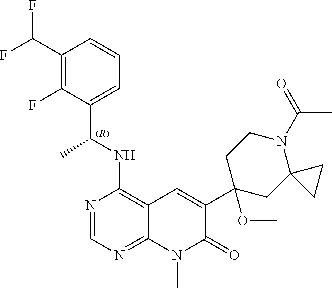

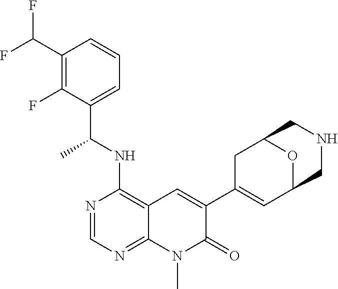 [SMILES: C[C@@H](Nc1ncnc2c1cc(C1=C[C@@H]3CNC[C@H](C1)O3)c(=O)n2C)c1cccc(C(F)F)c1F]

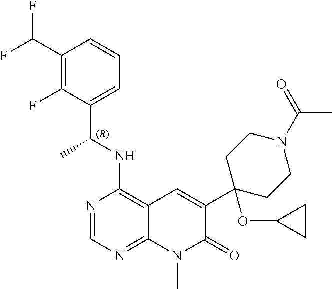

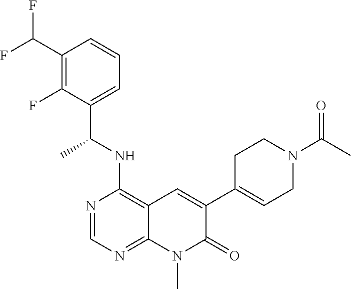 [SMILES: CC(=O)N1CC=C(c2cc3c(N[C@H](C)c4cccc(C(F)F)c4F)ncnc3n(C)c2=O)CC1]

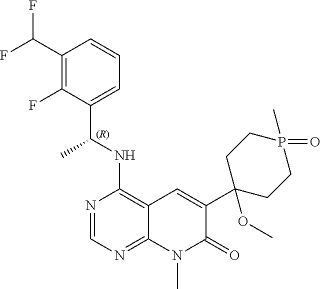

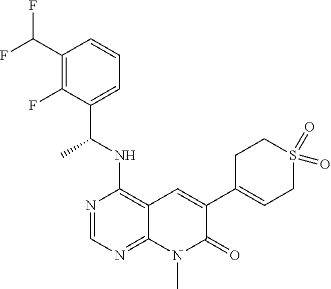 [SMILES: C[C@@H](Nc1ncnc2c1cc(C1=CCS(=O)(=O)CC1)c(=O)n2C)c1cccc(C(F)F)c1F]

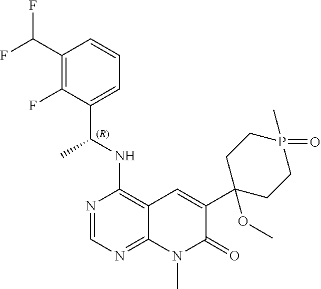

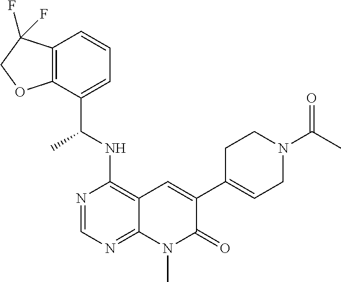 [SMILES: CC(=O)N1CC=C(c2cc3c(N[C@H](C)c4cccc5c4OCC5(F)F)ncnc3n(C)c2=O)CC1]

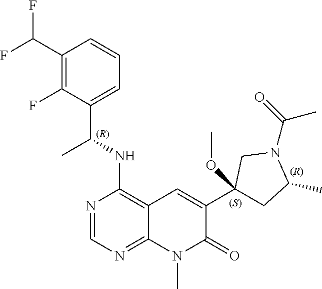

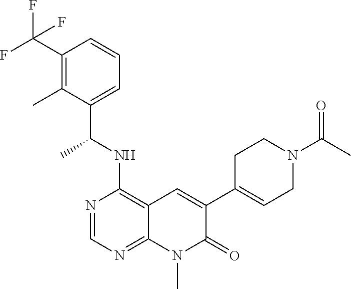 [SMILES: CC(=O)N1CC=C(c2cc3c(N[C@H](C)c4cccc(C(F)(F)F)c4C)ncnc3n(C)c2=O)CC1]

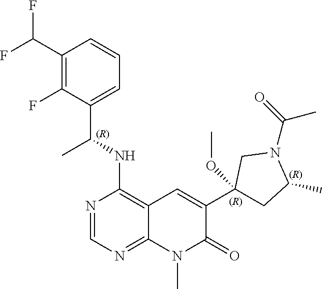

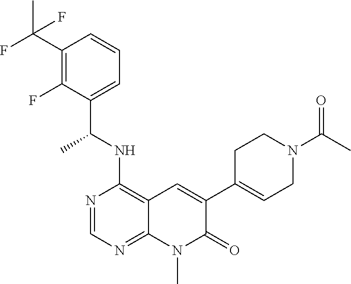 [SMILES: CC(=O)N1CC=C(c2cc3c(N[C@H](C)c4cccc(C(C)(F)F)c4F)ncnc3n(C)c2=O)CC1]

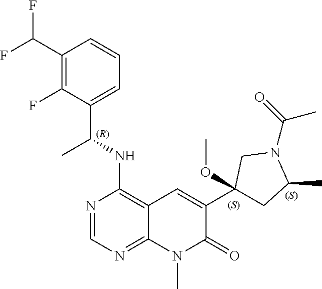

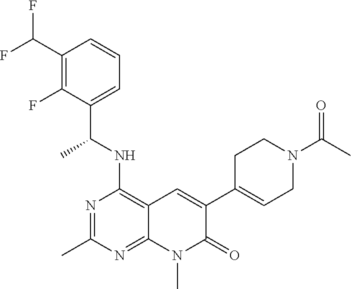 [SMILES: CC(=O)N1CC=C(c2cc3c(N[C@H](C)c4cccc(C(F)F)c4F)nc(C)nc3n(C)c2=O)CC1]

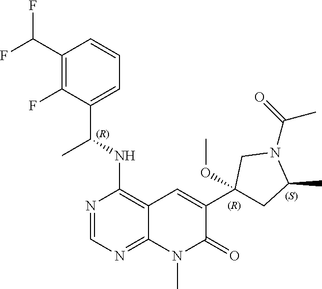

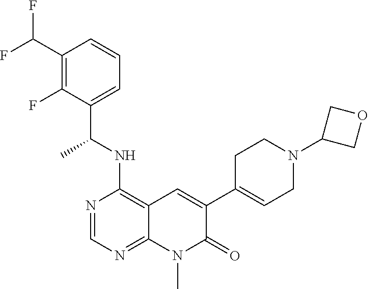 [SMILES: C[C@@H](Nc1ncnc2c1cc(C1=CCN(C3COC3)CC1)c(=O)n2C)c1cccc(C(F)F)c1F]

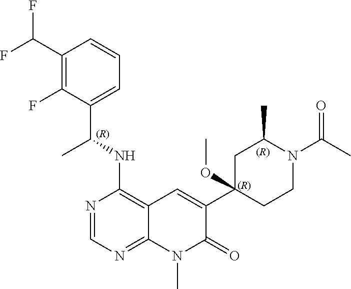

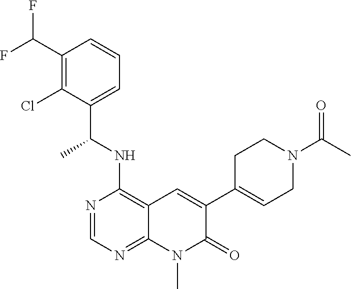 [SMILES: CC(=O)N1CC=C(c2cc3c(N[C@H](C)c4cccc(C(F)F)c4Cl)ncnc3n(C)c2=O)CC1]

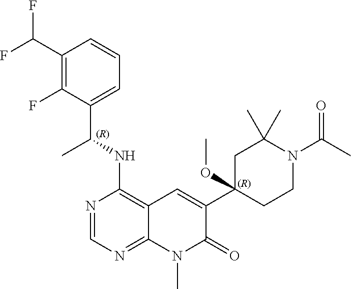

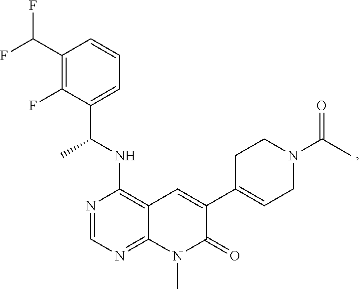 [SMILES: CC(=O)N1CC=C(c2cc3c(N[C@H](C)c4cccc(C(F)F)c4F)ncnc3n(C)c2=O)CC1]

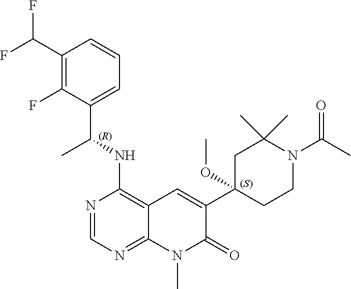

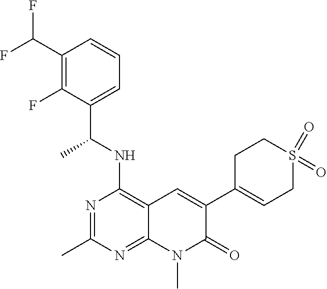 [SMILES: Cc1nc(N[C@H](C)c2cccc(C(F)F)c2F)c2cc(C3=CCS(=O)(=O)CC3)c(=O)n(C)c2n1]

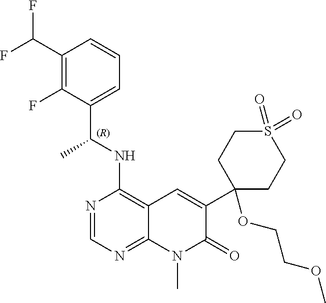

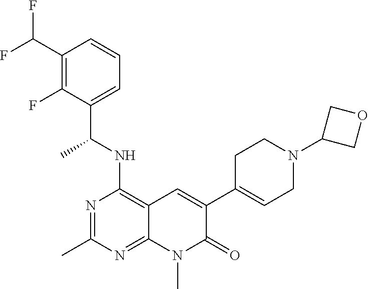 [SMILES: Cc1nc(N[C@H](C)c2cccc(C(F)F)c2F)c2cc(C3=CCN(C4COC4)CC3)c(=O)n(C)c2n1]

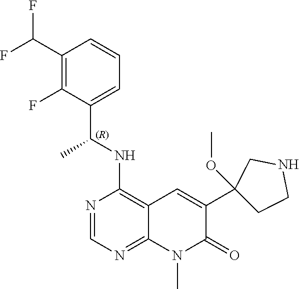

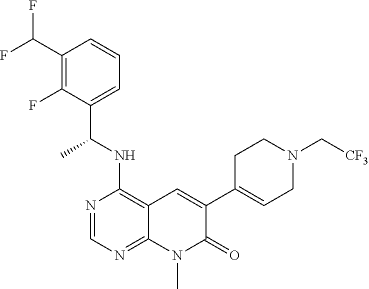 [SMILES: C[C@@H](Nc1ncnc2c1cc(C1=CCN(CC(F)(F)F)CC1)c(=O)n2C)c1cccc(C(F)F)c1F]

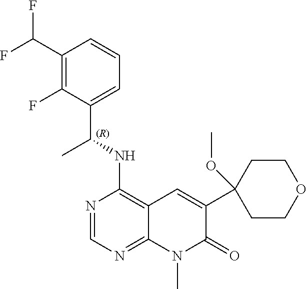

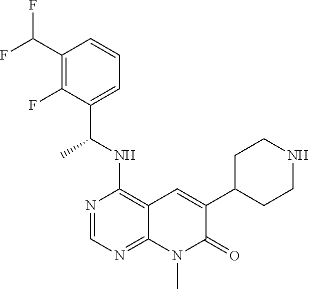 [SMILES: C[C@@H](Nc1ncnc2c1cc(C1CCNCC1)c(=O)n2C)c1cccc(C(F)F)c1F]

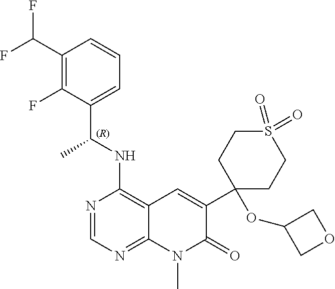

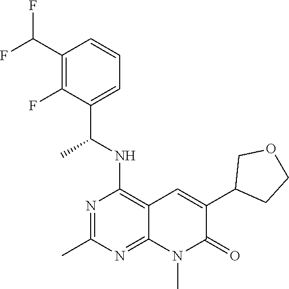 [SMILES: Cc1nc(N[C@H](C)c2cccc(C(F)F)c2F)c2cc(C3CCOC3)c(=O)n(C)c2n1]

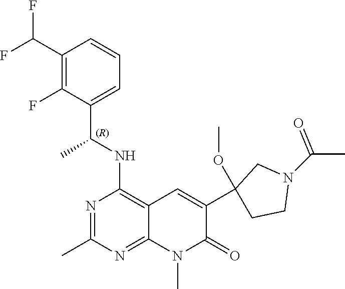

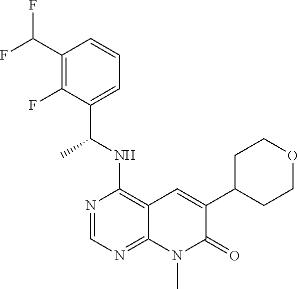 [SMILES: C[C@@H](Nc1ncnc2c1cc(C1CCOCC1)c(=O)n2C)c1cccc(C(F)F)c1F]

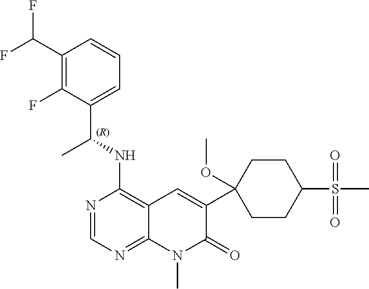

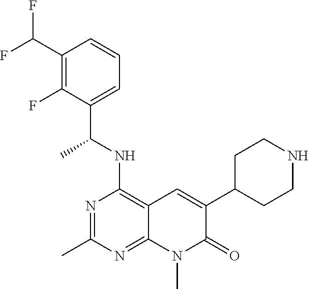 [SMILES: Cc1nc(N[C@H](C)c2cccc(C(F)F)c2F)c2cc(C3CCNCC3)c(=O)n(C)c2n1]

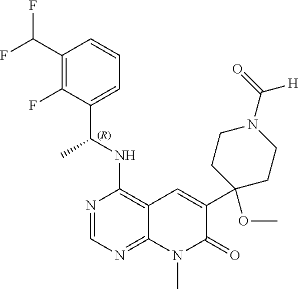

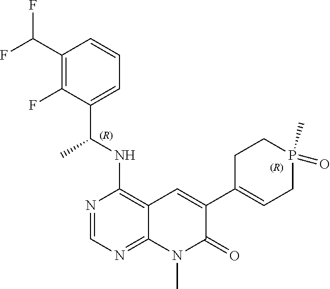

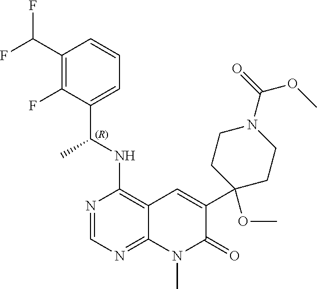

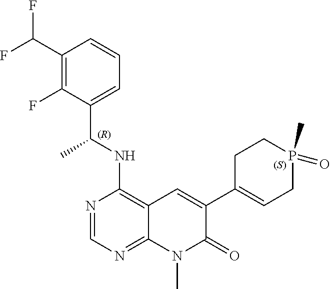

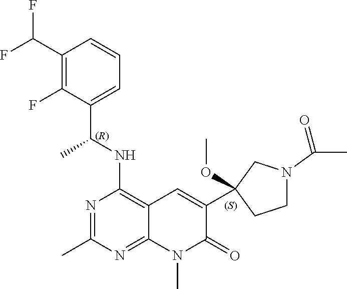

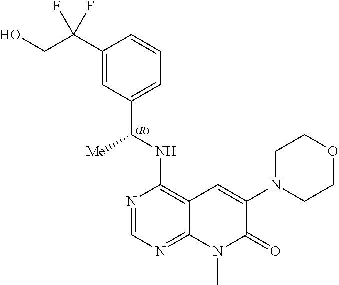

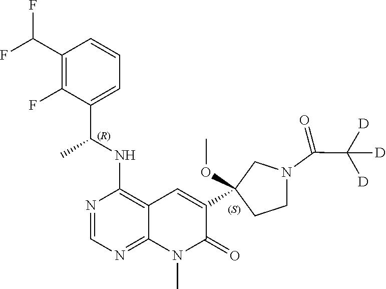

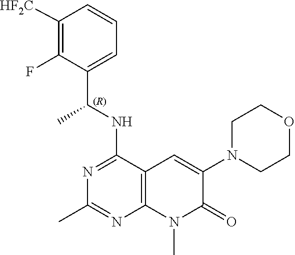

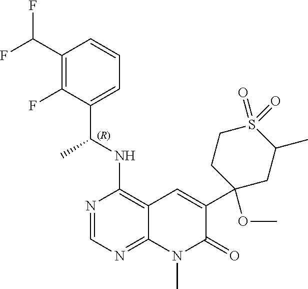

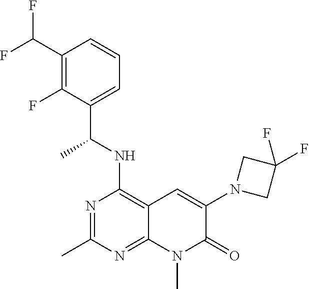 [SMILES: Cc1nc(N[C@H](C)c2cccc(C(F)F)c2F)c2cc(N3CC(F)(F)C3)c(=O)n(C)c2n1]

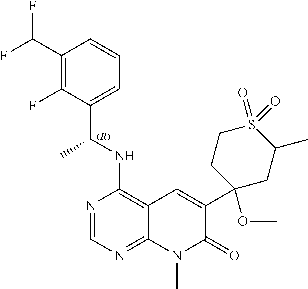

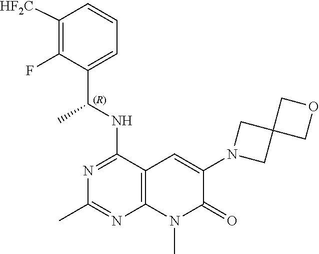

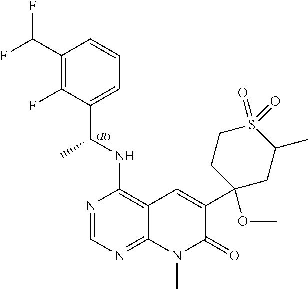

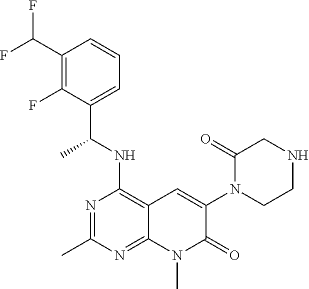 [SMILES: Cc1nc(N[C@H](C)c2cccc(C(F)F)c2F)c2cc(N3CCNCC3=O)c(=O)n(C)c2n1]

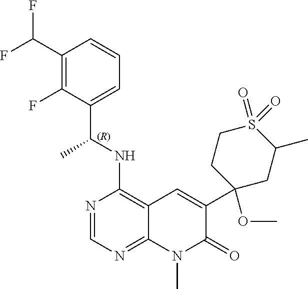

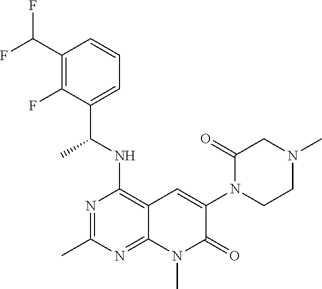 [SMILES: Cc1nc(N[C@H](C)c2cccc(C(F)F)c2F)c2cc(N3CCN(C)CC3=O)c(=O)n(C)c2n1]

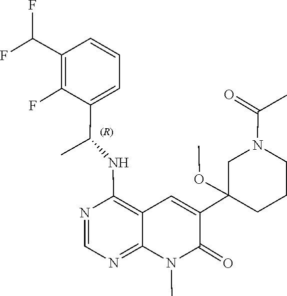

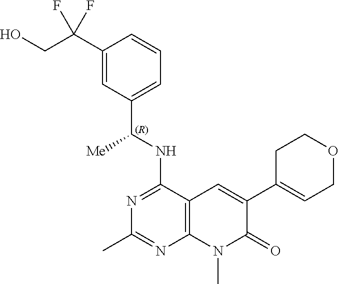

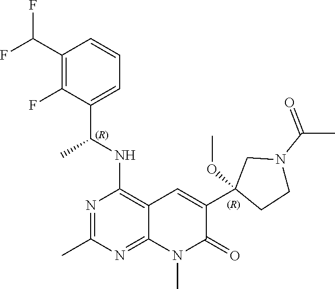

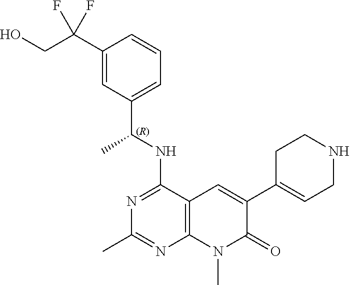

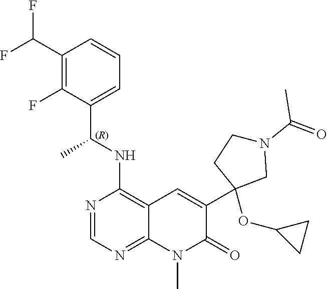

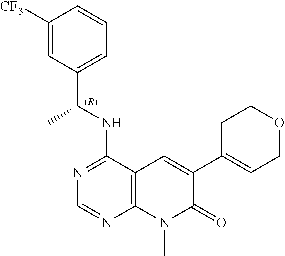

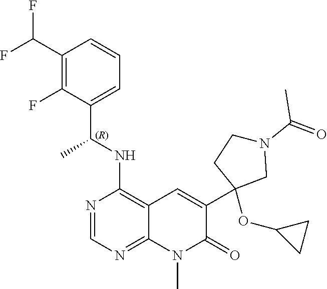

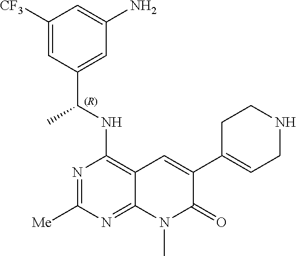

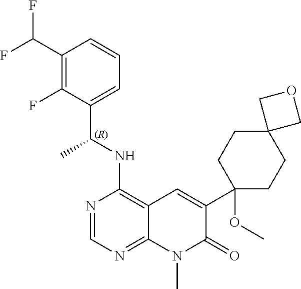

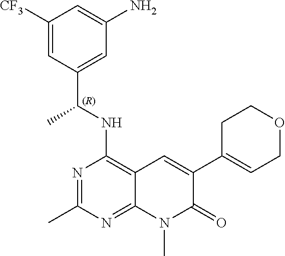

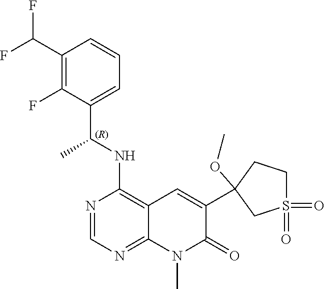

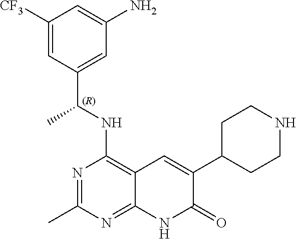

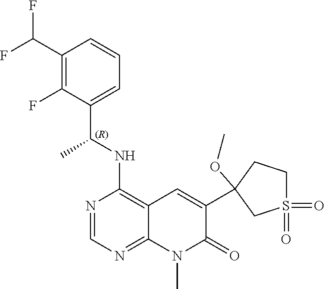

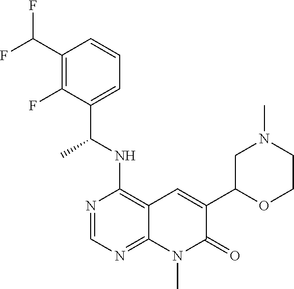 [SMILES: C[C@@H](Nc1ncnc2c1cc(C1CN(C)CCO1)c(=O)n2C)c1cccc(C(F)F)c1F]

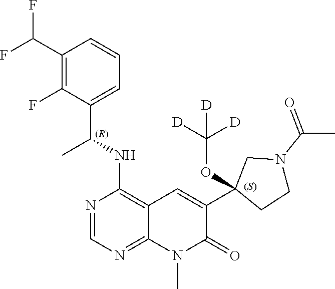

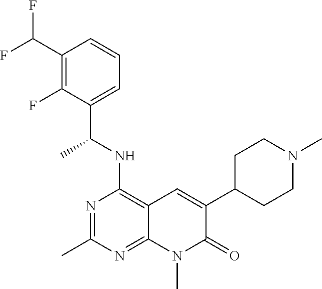 [SMILES: Cc1nc(N[C@H](C)c2cccc(C(F)F)c2F)c2cc(C3CCN(C)CC3)c(=O)n(C)c2n1]

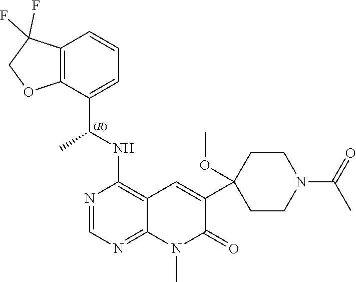

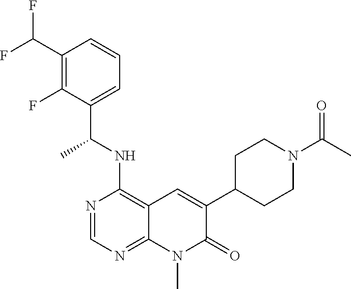 [SMILES: CC(=O)N1CCC(c2cc3c(N[C@H](C)c4cccc(C(F)F)c4F)ncnc3n(C)c2=O)CC1]

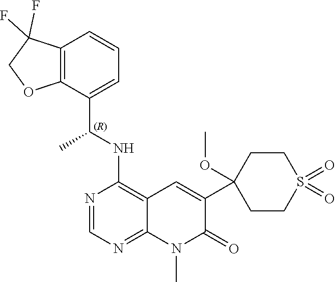

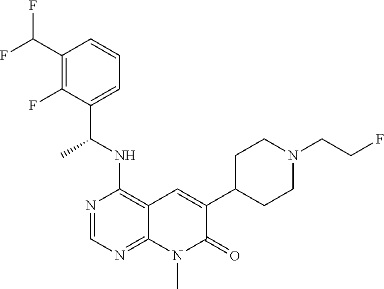 [SMILES: C[C@@H](Nc1ncnc2c1cc(C1CCN(CCF)CC1)c(=O)n2C)c1cccc(C(F)F)c1F]

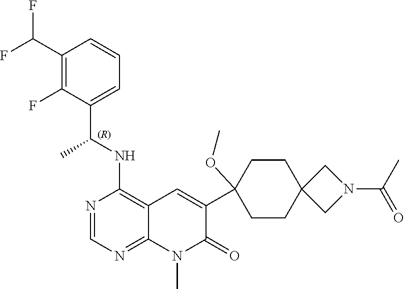

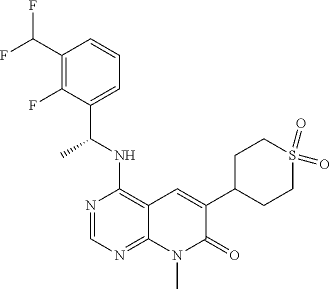 [SMILES: C[C@@H](Nc1ncnc2c1cc(C1CCS(=O)(=O)CC1)c(=O)n2C)c1cccc(C(F)F)c1F]

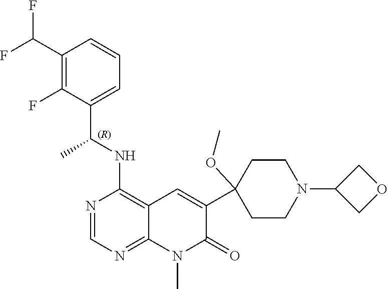

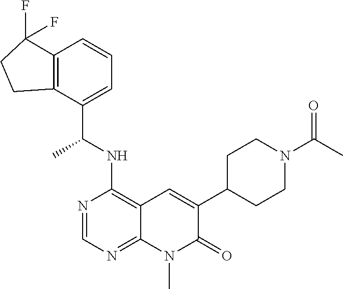 [SMILES: CC(=O)N1CCC(c2cc3c(N[C@H](C)c4cccc5c4CCC5(F)F)ncnc3n(C)c2=O)CC1]

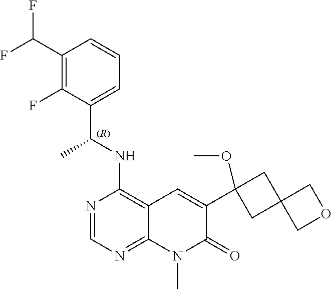

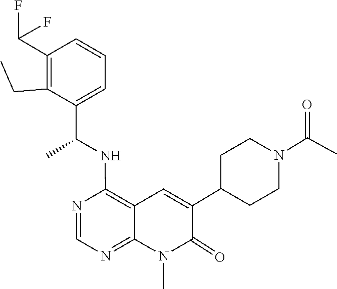 [SMILES: CCc1c(C(F)F)cccc1[C@@H](C)Nc1ncnc2c1cc(C1CCN(C(C)=O)CC1)c(=O)n2C]

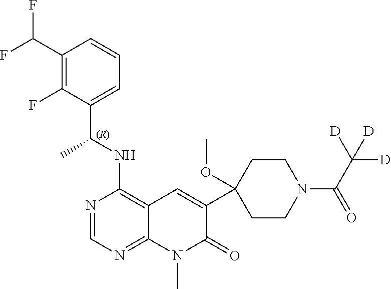

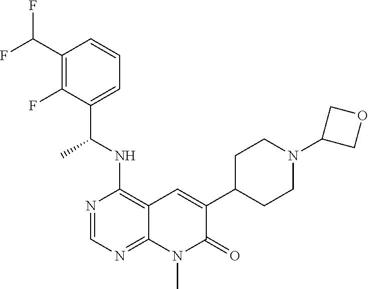 [SMILES: C[C@@H](Nc1ncnc2c1cc(C1CCN(C3COC3)CC1)c(=O)n2C)c1cccc(C(F)F)c1F]

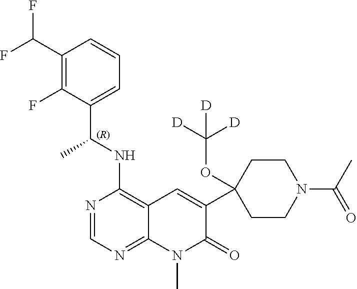

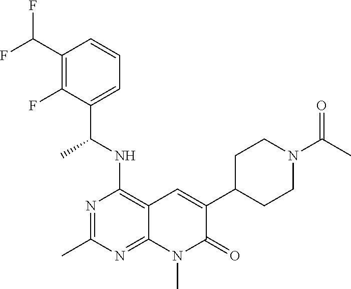 [SMILES: CC(=O)N1CCC(c2cc3c(N[C@H](C)c4cccc(C(F)F)c4F)nc(C)nc3n(C)c2=O)CC1]

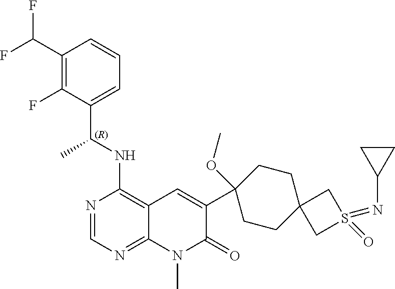

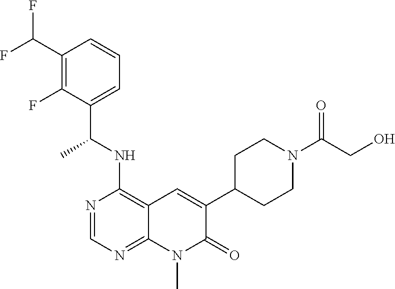 [SMILES: C[C@@H](Nc1ncnc2c1cc(C1CCN(C(=O)CO)CC1)c(=O)n2C)c1cccc(C(F)F)c1F]

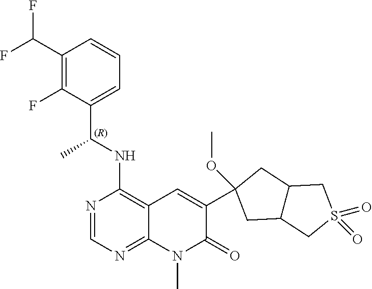

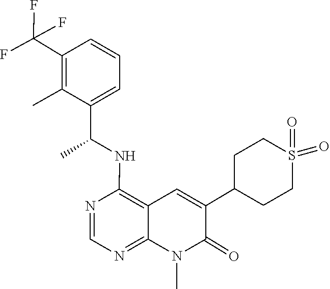 [SMILES: Cc1c([C@@H](C)Nc2ncnc3c2cc(C2CCS(=O)(=O)CC2)c(=O)n3C)cccc1C(F)(F)F]

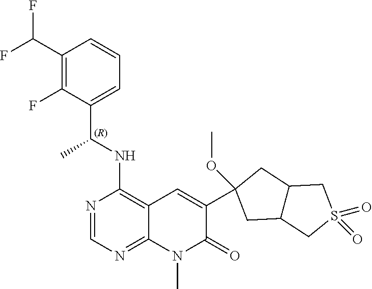

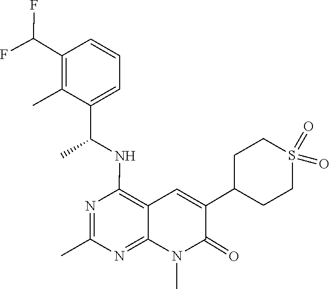 [SMILES: Cc1nc(N[C@H](C)c2cccc(C(F)F)c2C)c2cc(C3CCS(=O)(=O)CC3)c(=O)n(C)c2n1]

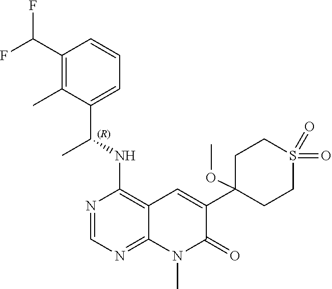

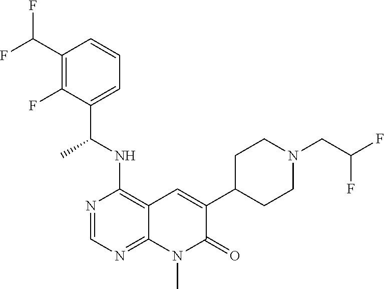 [SMILES: C[C@@H](Nc1ncnc2c1cc(C1CCN(CC(F)F)CC1)c(=O)n2C)c1cccc(C(F)F)c1F]

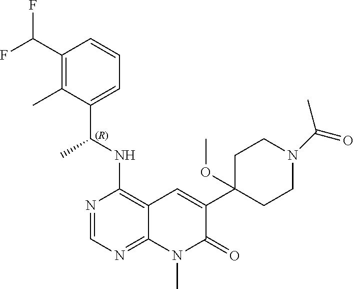

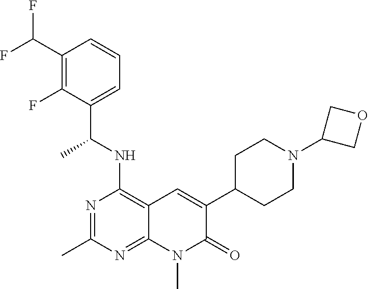 [SMILES: Cc1nc(N[C@H](C)c2cccc(C(F)F)c2F)c2cc(C3CCN(C4COC4)CC3)c(=O)n(C)c2n1]

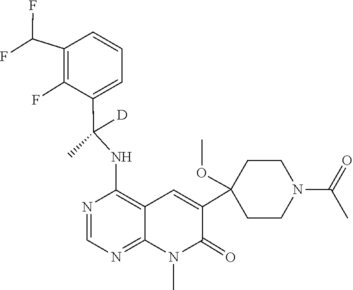 [SMILES: [2H][C@@](C)(Nc1ncnc2c1cc(C1(OC)CCN(C(C)=O)CC1)c(=O)n2C)c1cccc(C(F)F)c1F]

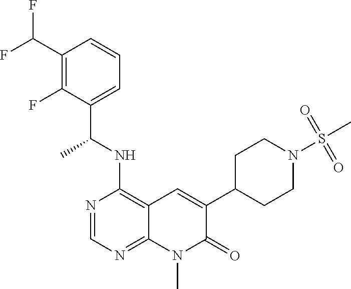 [SMILES: C[C@@H](Nc1ncnc2c1cc(C1CCN(S(C)(=O)=O)CC1)c(=O)n2C)c1cccc(C(F)F)c1F]

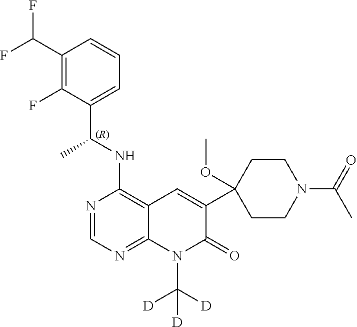

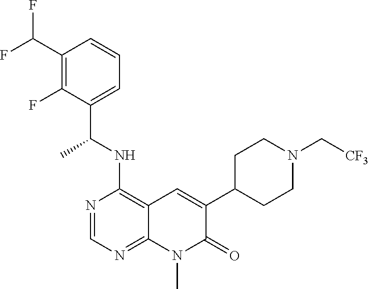 [SMILES: C[C@@H](Nc1ncnc2c1cc(C1CCN(CC(F)(F)F)CC1)c(=O)n2C)c1cccc(C(F)F)c1F]

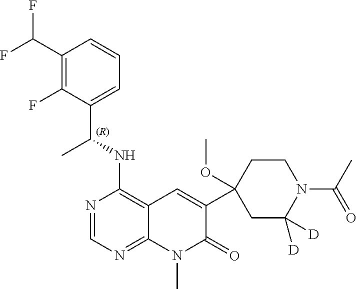

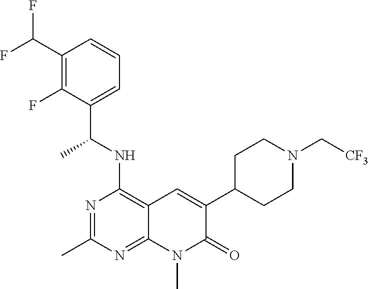 [SMILES: Cc1nc(N[C@H](C)c2cccc(C(F)F)c2F)c2cc(C3CCN(CC(F)(F)F)CC3)c(=O)n(C)c2n1]

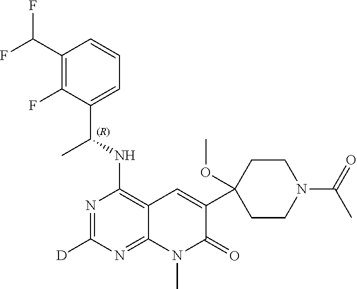

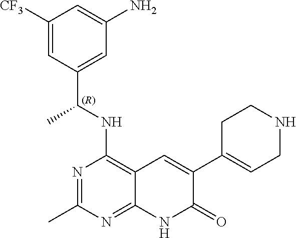

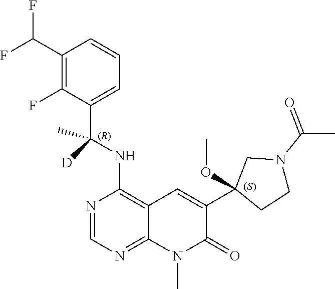

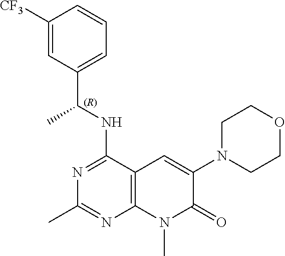

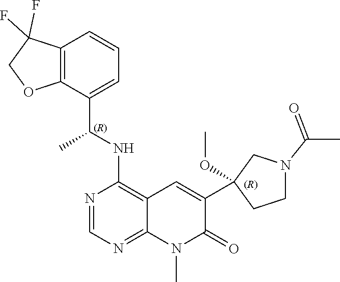

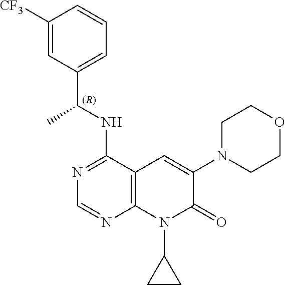

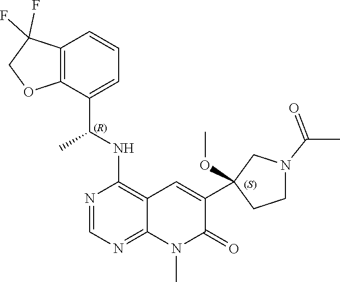

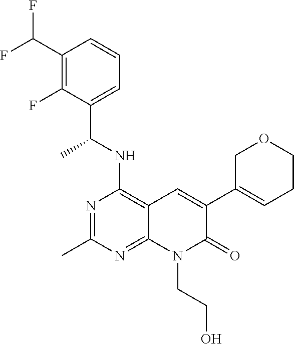 [SMILES: Cc1nc(N[C@H](C)c2cccc(C(F)F)c2F)c2cc(C3=CCCOC3)c(=O)n(CCO)c2n1]

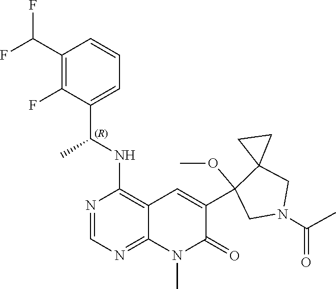

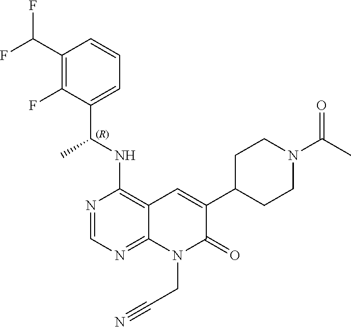

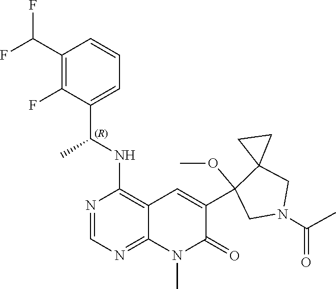

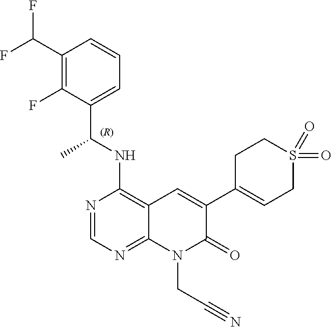

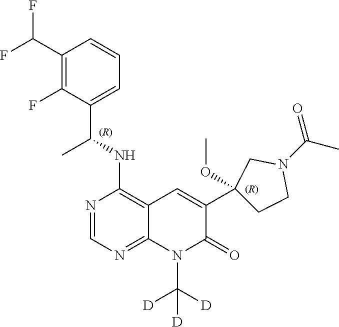

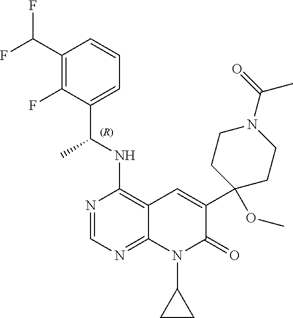

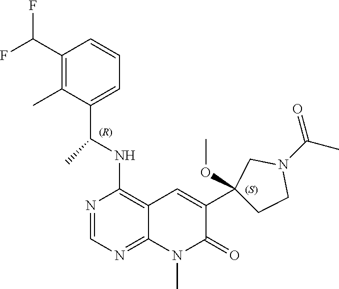

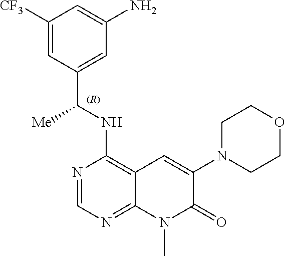

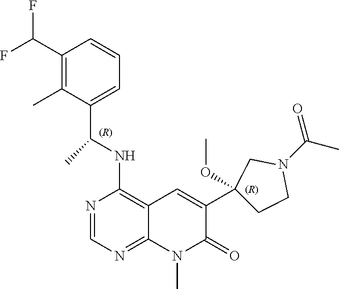

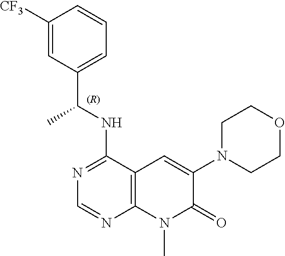

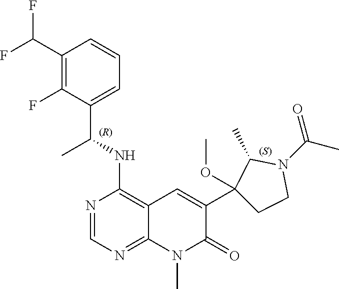

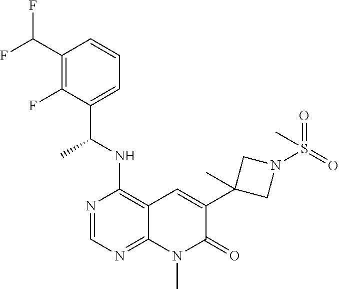 [SMILES: C[C@@H](Nc1ncnc2c1cc(C1(C)CN(S(C)(=O)=O)C1)c(=O)n2C)c1cccc(C(F)F)c1F]

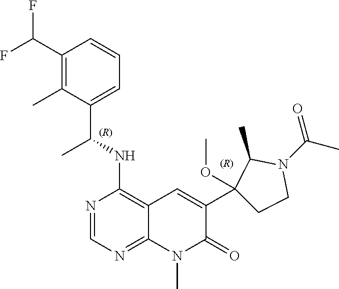

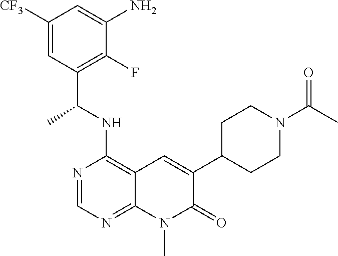 [SMILES: CC(=O)N1CCC(c2cc3c(N[C@H](C)c4cc(C(F)(F)F)cc(N)c4F)ncnc3n(C)c2=O)CC1]

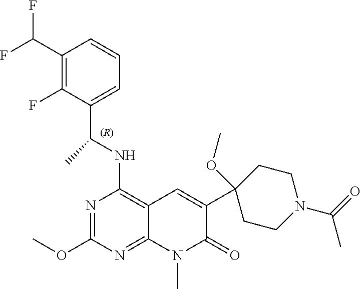

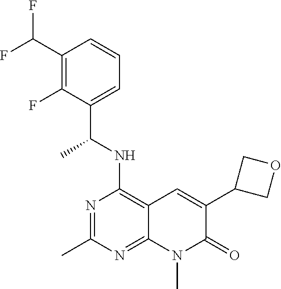 [SMILES: Cc1nc(N[C@H](C)c2cccc(C(F)F)c2F)c2cc(C3COC3)c(=O)n(C)c2n1]

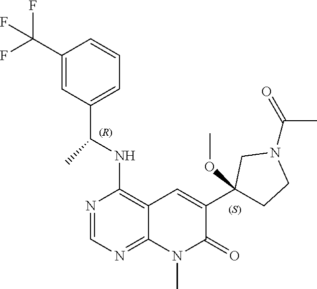

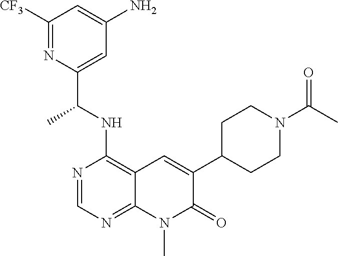 [SMILES: CC(=O)N1CCC(c2cc3c(N[C@H](C)c4cc(N)cc(C(F)(F)F)n4)ncnc3n(C)c2=O)CC1]

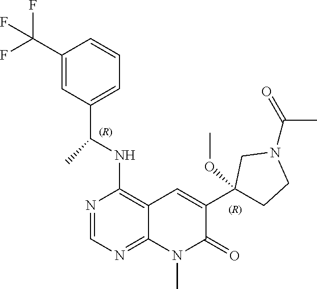

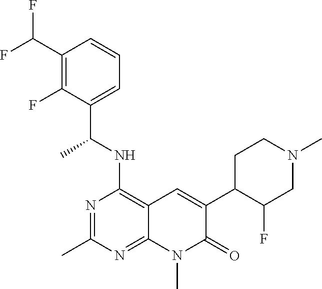 [SMILES: Cc1nc(N[C@H](C)c2cccc(C(F)F)c2F)c2cc(C3CCN(C)CC3F)c(=O)n(C)c2n1]

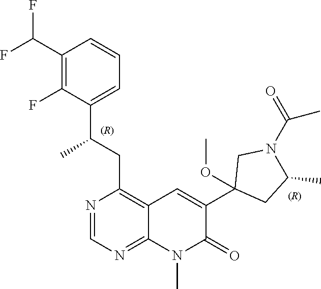

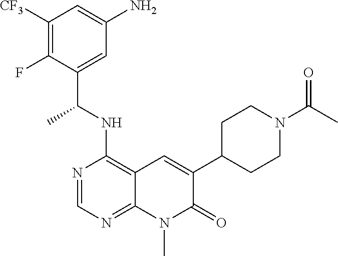 [SMILES: CC(=O)N1CCC(c2cc3c(N[C@H](C)c4cc(N)cc(C(F)(F)F)c4F)ncnc3n(C)c2=O)CC1]

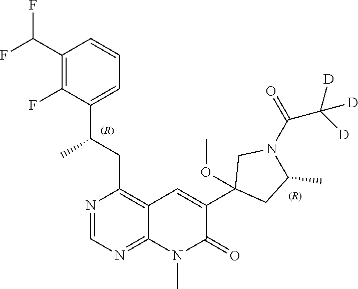

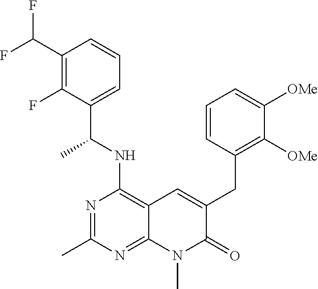 [SMILES: COc1cccc(Cc2cc3c(N[C@H](C)c4cccc(C(F)F)c4F)nc(C)nc3n(C)c2=O)c1OC]

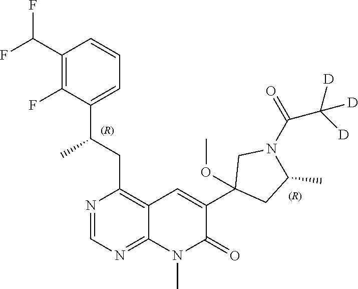

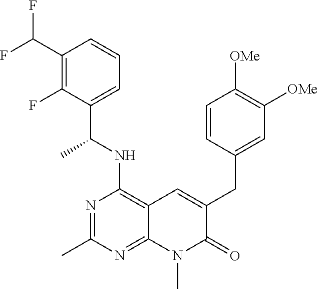 [SMILES: COc1ccc(Cc2cc3c(N[C@H](C)c4cccc(C(F)F)c4F)nc(C)nc3n(C)c2=O)cc1OC]

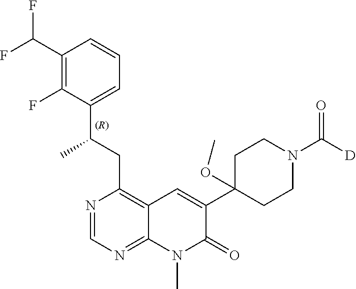

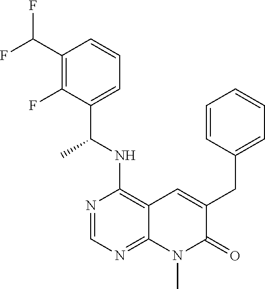 [SMILES: C[C@@H](Nc1ncnc2c1cc(Cc1ccccc1)c(=O)n2C)c1cccc(C(F)F)c1F]

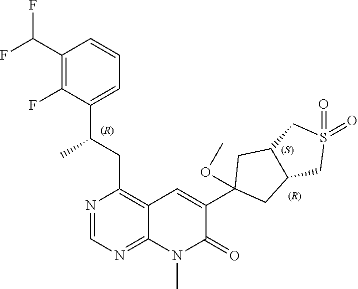

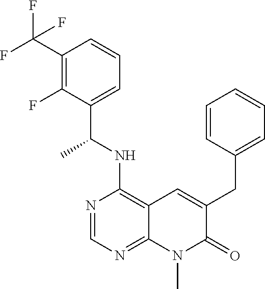 [SMILES: C[C@@H](Nc1ncnc2c1cc(Cc1ccccc1)c(=O)n2C)c1cccc(C(F)(F)F)c1F]

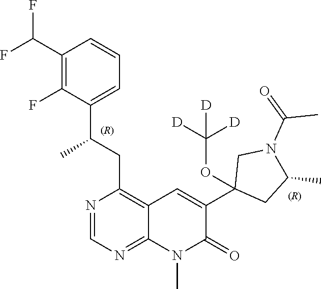

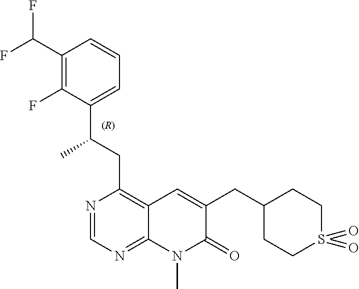

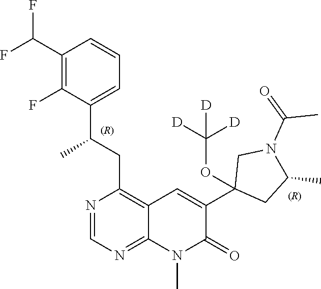

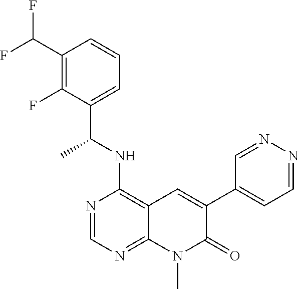 [SMILES: C[C@@H](Nc1ncnc2c1cc(-c1ccnnc1)c(=O)n2C)c1cccc(C(F)F)c1F]

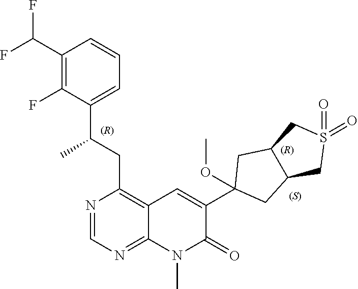

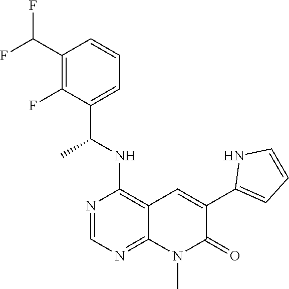 [SMILES: C[C@@H](Nc1ncnc2c1cc(-c1ccc[nH]1)c(=O)n2C)c1cccc(C(F)F)c1F]

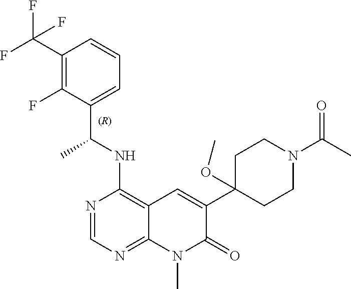

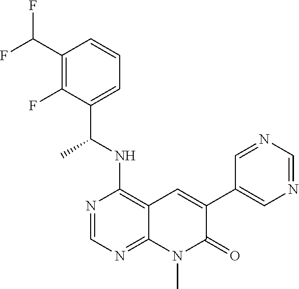 [SMILES: C[C@@H](Nc1ncnc2c1cc(-c1cncnc1)c(=O)n2C)c1cccc(C(F)F)c1F]

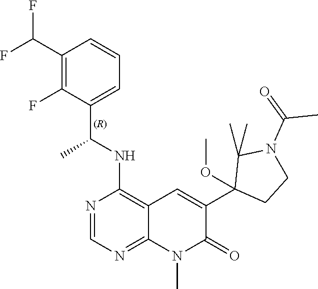

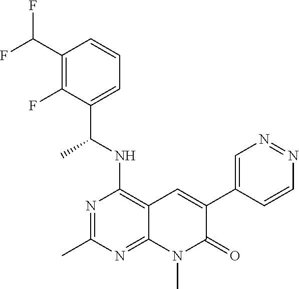 [SMILES: Cc1nc(N[C@H](C)c2cccc(C(F)F)c2F)c2cc(-c3ccnnc3)c(=O)n(C)c2n1]

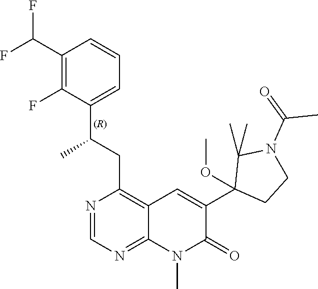

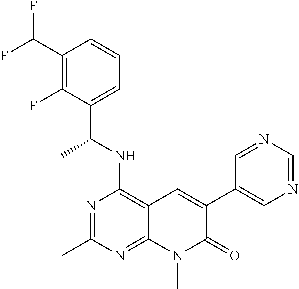 [SMILES: Cc1nc(N[C@H](C)c2cccc(C(F)F)c2F)c2cc(-c3cncnc3)c(=O)n(C)c2n1]

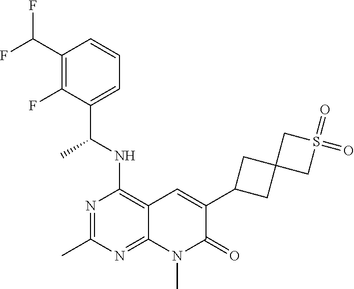 [SMILES: Cc1nc(N[C@H](C)c2cccc(C(F)F)c2F)c2cc(C3CC4(C3)CS(=O)(=O)C4)c(=O)n(C)c2n1]

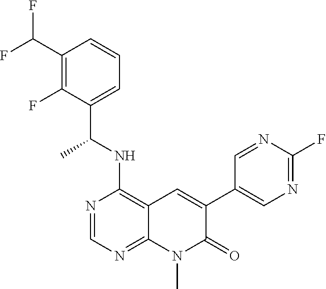 [SMILES: C[C@@H](Nc1ncnc2c1cc(-c1cnc(F)nc1)c(=O)n2C)c1cccc(C(F)F)c1F]

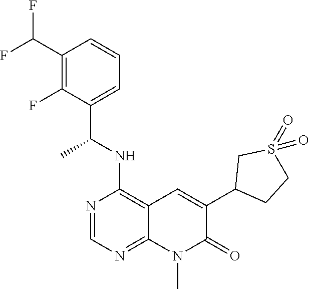 [SMILES: C[C@@H](Nc1ncnc2c1cc(C1CCS(=O)(=O)C1)c(=O)n2C)c1cccc(C(F)F)c1F]

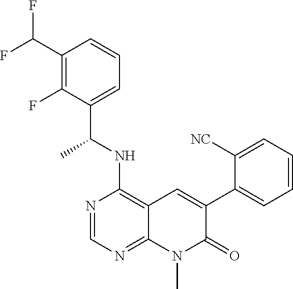 [SMILES: C[C@@H](Nc1ncnc2c1cc(-c1ccccc1C#N)c(=O)n2C)c1cccc(C(F)F)c1F]

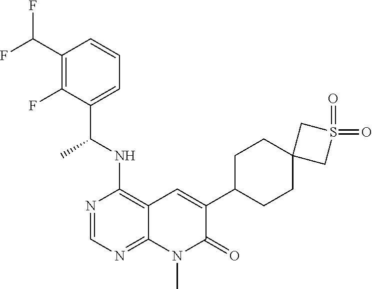 [SMILES: C[C@@H](Nc1ncnc2c1cc(C1CCC3(CC1)CS(=O)(=O)C3)c(=O)n2C)c1cccc(C(F)F)c1F]

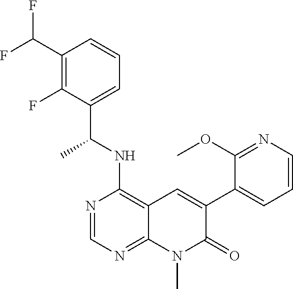 [SMILES: COc1ncccc1-c1cc2c(N[C@H](C)c3cccc(C(F)F)c3F)ncnc2n(C)c1=O]

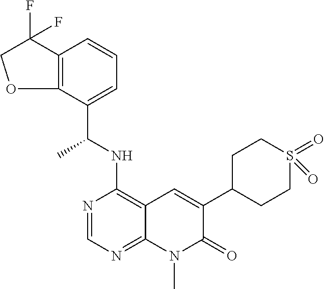 [SMILES: C[C@@H](Nc1ncnc2c1cc(C1CCS(=O)(=O)CC1)c(=O)n2C)c1cccc2c1OCC2(F)F]

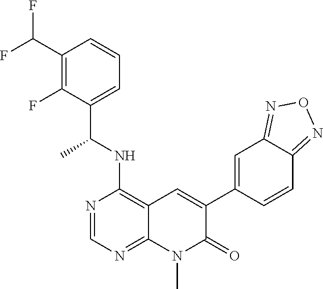 [SMILES: C[C@@H](Nc1ncnc2c1cc(-c1ccc3nonc3c1)c(=O)n2C)c1cccc(C(F)F)c1F]

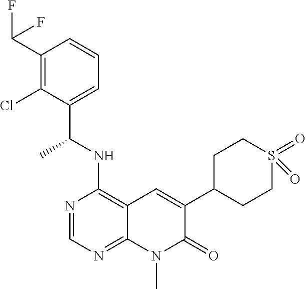 [SMILES: C[C@@H](Nc1ncnc2c1cc(C1CCS(=O)(=O)CC1)c(=O)n2C)c1cccc(C(F)F)c1Cl]

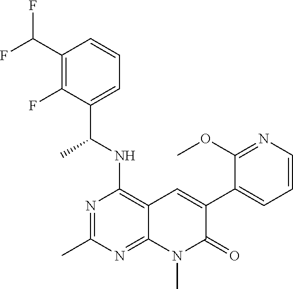 [SMILES: COc1ncccc1-c1cc2c(N[C@H](C)c3cccc(C(F)F)c3F)nc(C)nc2n(C)c1=O]

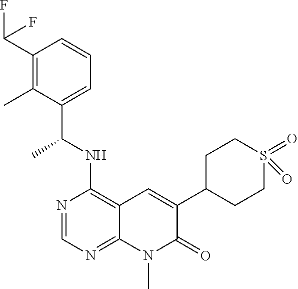 [SMILES: Cc1c(C(F)F)cccc1[C@@H](C)Nc1ncnc2c1cc(C1CCS(=O)(=O)CC1)c(=O)n2C]

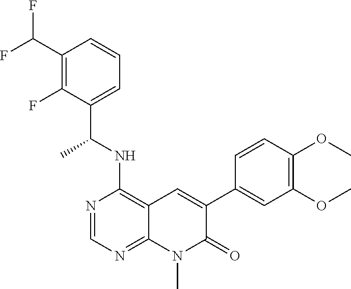 [SMILES: COc1ccc(-c2cc3c(N[C@H](C)c4cccc(C(F)F)c4F)ncnc3n(C)c2=O)cc1OC]

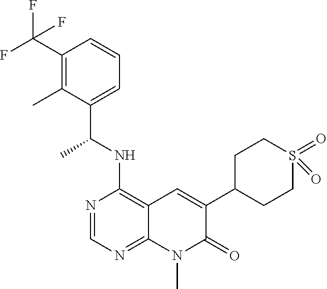 [SMILES: Cc1c([C@@H](C)Nc2ncnc3c2cc(C2CCS(=O)(=O)CC2)c(=O)n3C)cccc1C(F)(F)F]

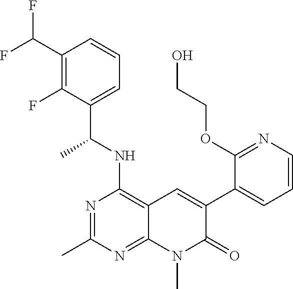 [SMILES: Cc1nc(N[C@H](C)c2cccc(C(F)F)c2F)c2cc(-c3cccnc3OCCO)c(=O)n(C)c2n1]

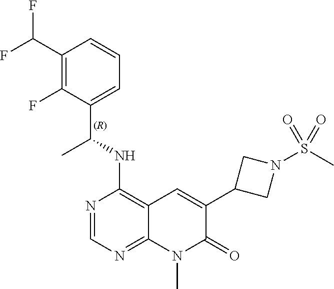

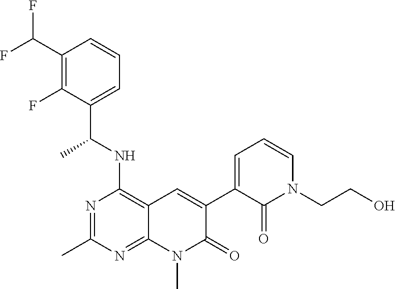 [SMILES: Cc1nc(N[C@H](C)c2cccc(C(F)F)c2F)c2cc(-c3cccn(CCO)c3=O)c(=O)n(C)c2n1]

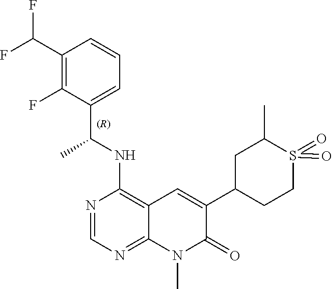

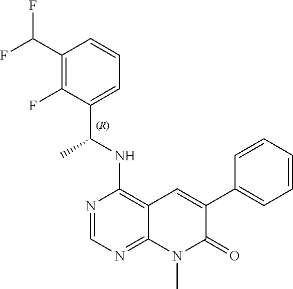

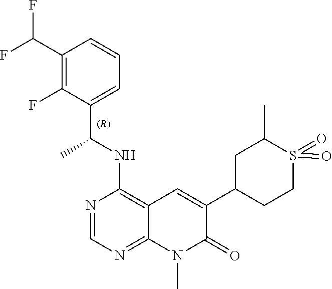

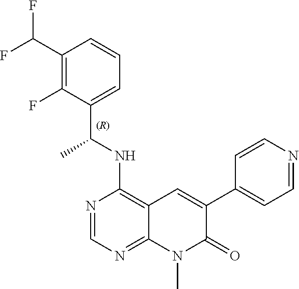

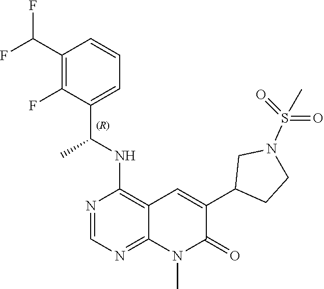

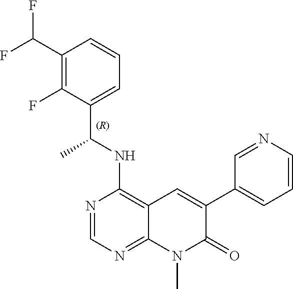

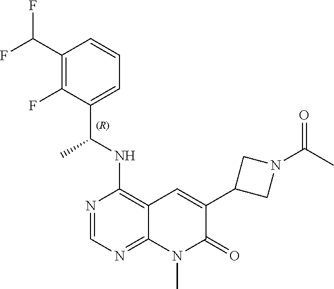

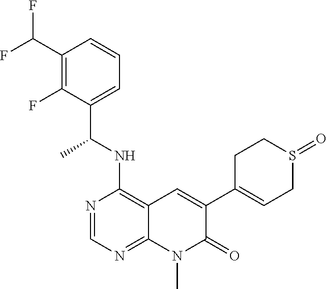 [SMILES: C[C@@H](Nc1ncnc2c1cc(C1=CCS(=O)CC1)c(=O)n2C)c1cccc(C(F)F)c1F]

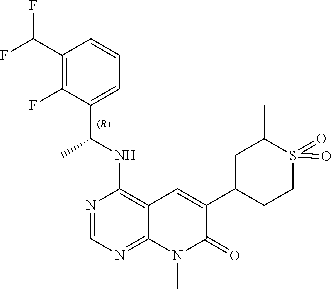

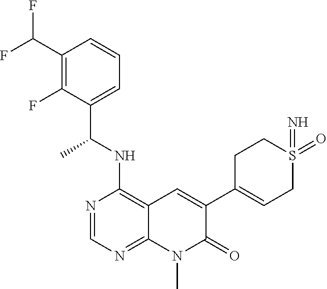 [SMILES: C[C@@H](Nc1ncnc2c1cc(C1=CCS(=N)(=O)CC1)c(=O)n2C)c1cccc(C(F)F)c1F]

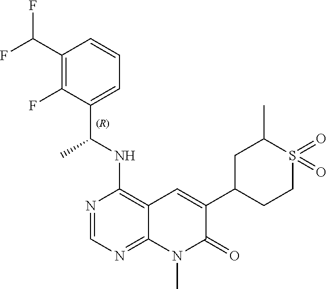

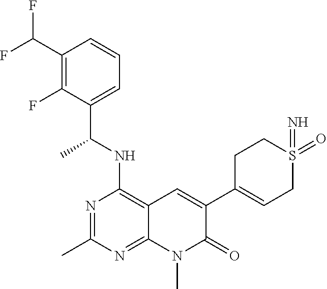 [SMILES: Cc1nc(N[C@H](C)c2cccc(C(F)F)c2F)c2cc(C3=CCS(=N)(=O)CC3)c(=O)n(C)c2n1]

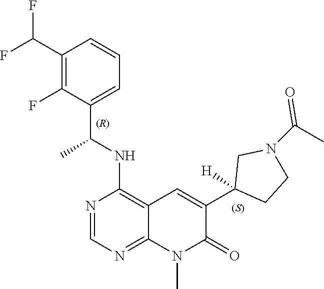

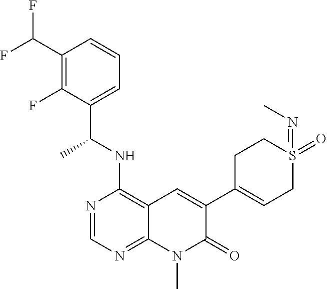 [SMILES: CN=S1(=O)CC=C(c2cc3c(N[C@H](C)c4cccc(C(F)F)c4F)ncnc3n(C)c2=O)CC1]

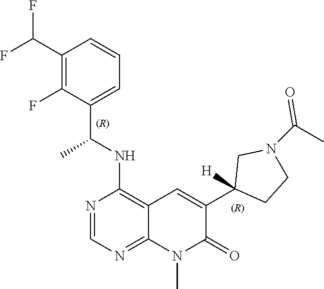

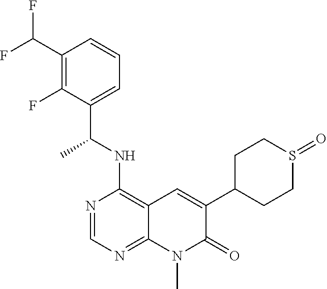 [SMILES: C[C@@H](Nc1ncnc2c1cc(C1CCS(=O)CC1)c(=O)n2C)c1cccc(C(F)F)c1F]

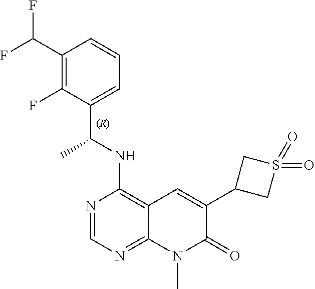

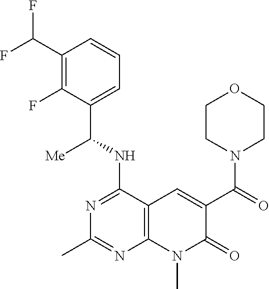 [SMILES: Cc1nc(N[C@H](C)c2cccc(C(F)F)c2F)c2cc(C(=O)N3CCOCC3)c(=O)n(C)c2n1]

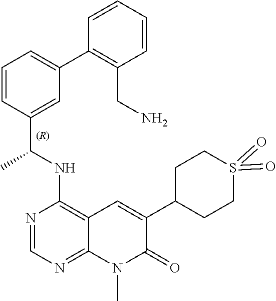

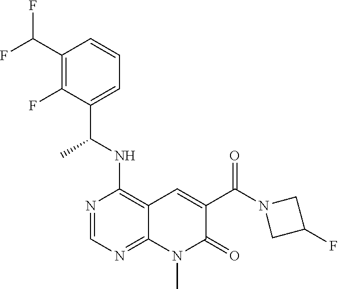 [SMILES: C[C@@H](Nc1ncnc2c1cc(C(=O)N1CC(F)C1)c(=O)n2C)c1cccc(C(F)F)c1F]

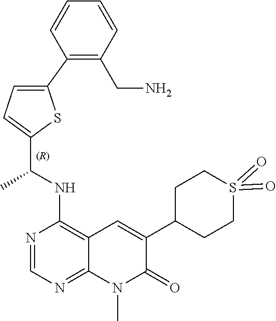

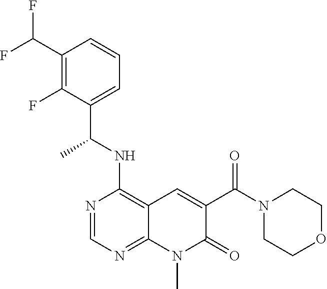 [SMILES: C[C@@H](Nc1ncnc2c1cc(C(=O)N1CCOCC1)c(=O)n2C)c1cccc(C(F)F)c1F]

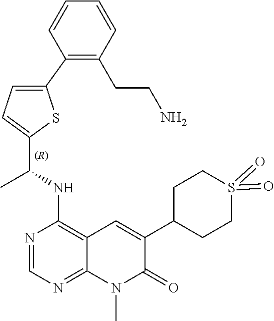

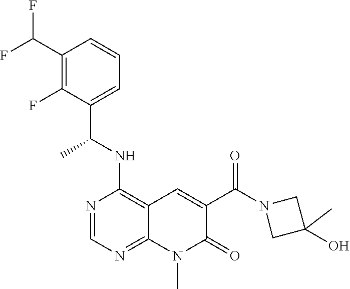 [SMILES: C[C@@H](Nc1ncnc2c1cc(C(=O)N1CC(C)(O)C1)c(=O)n2C)c1cccc(C(F)F)c1F]

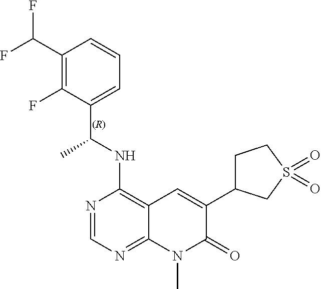

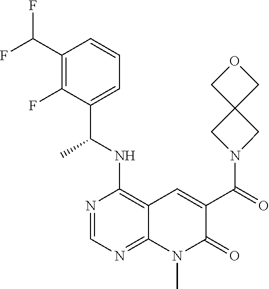 [SMILES: C[C@@H](Nc1ncnc2c1cc(C(=O)N1CC3(COC3)C1)c(=O)n2C)c1cccc(C(F)F)c1F]

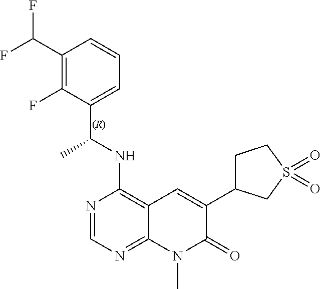

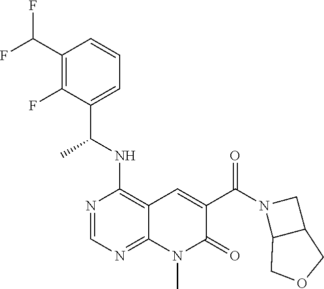 [SMILES: C[C@@H](Nc1ncnc2c1cc(C(=O)N1CC3COCC31)c(=O)n2C)c1cccc(C(F)F)c1F]

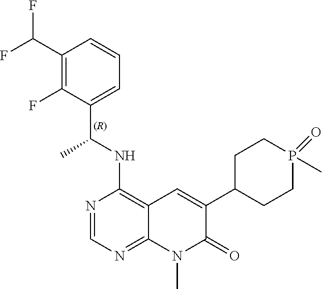

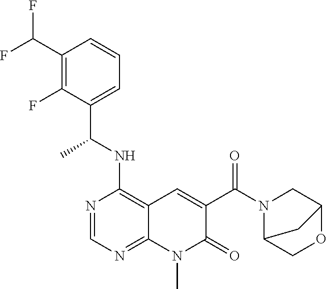 [SMILES: C[C@@H](Nc1ncnc2c1cc(C(=O)N1CC3CC1CO3)c(=O)n2C)c1cccc(C(F)F)c1F]

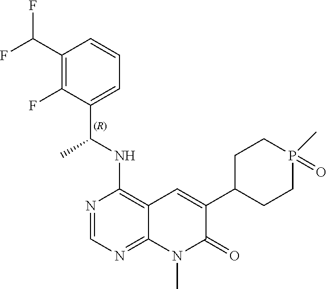

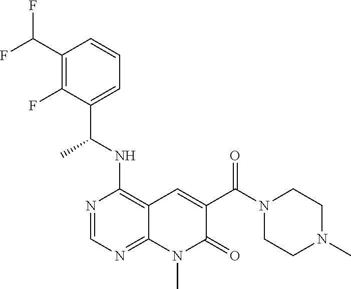 [SMILES: C[C@@H](Nc1ncnc2c1cc(C(=O)N1CCN(C)CC1)c(=O)n2C)c1cccc(C(F)F)c1F]

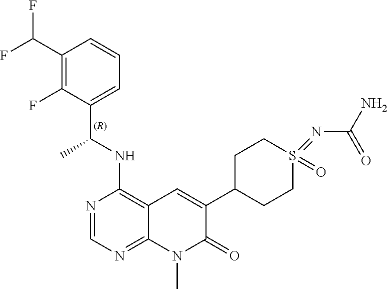

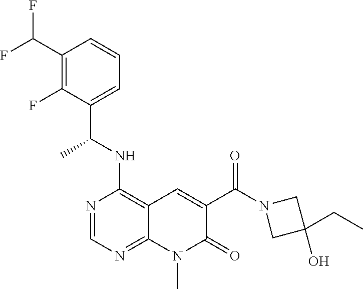 [SMILES: CCC1(O)CN(C(=O)c2cc3c(N[C@H](C)c4cccc(C(F)F)c4F)ncnc3n(C)c2=O)C1]

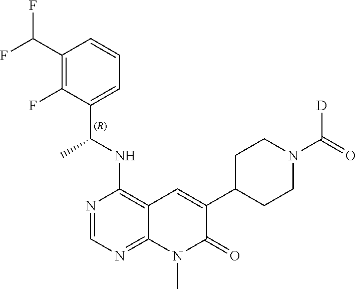

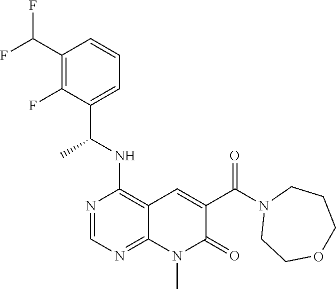 [SMILES: C[C@@H](Nc1ncnc2c1cc(C(=O)N1CCCOCC1)c(=O)n2C)c1cccc(C(F)F)c1F]

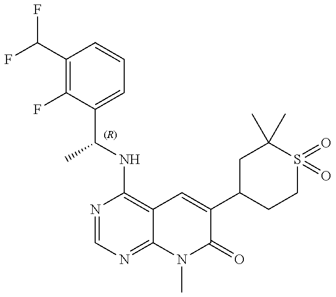

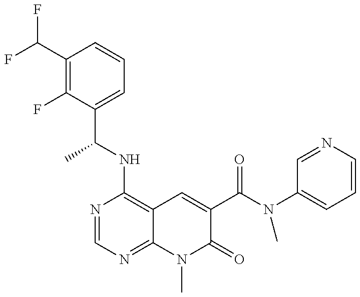 [SMILES: C[C@@H](Nc1ncnc2c1cc(C(=O)N(C)c1cccnc1)c(=O)n2C)c1cccc(C(F)F)c1F]

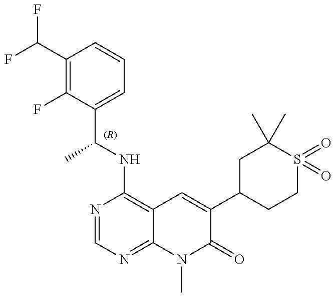

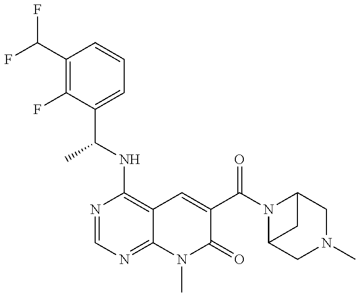 [SMILES: C[C@@H](Nc1ncnc2c1cc(C(=O)N1C3CC1CN(C)C3)c(=O)n2C)c1cccc(C(F)F)c1F]

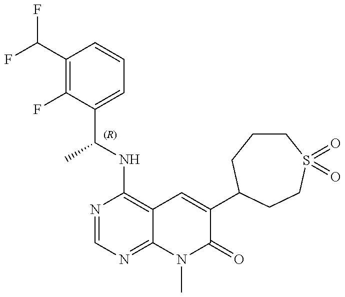

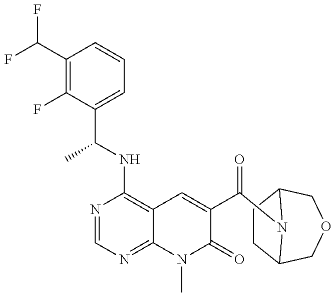 [SMILES: C[C@@H](Nc1ncnc2c1cc(C(=O)N1C3CCC1COC3)c(=O)n2C)c1cccc(C(F)F)c1F]

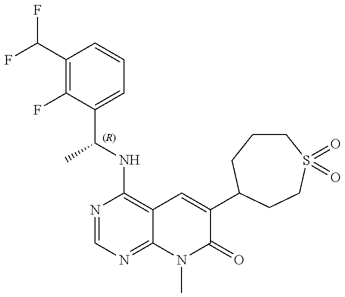

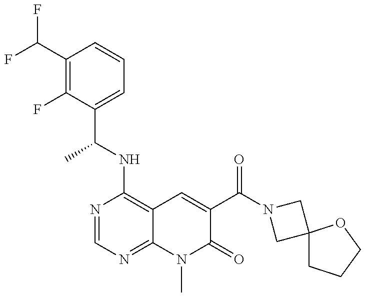 [SMILES: C[C@@H](Nc1ncnc2c1cc(C(=O)N1CC3(CCCO3)C1)c(=O)n2C)c1cccc(C(F)F)c1F]

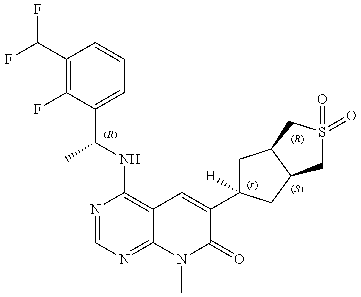

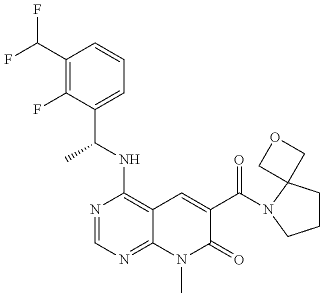 [SMILES: C[C@@H](Nc1ncnc2c1cc(C(=O)N1CCCC13COC3)c(=O)n2C)c1cccc(C(F)F)c1F]

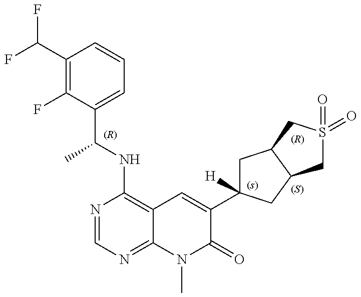

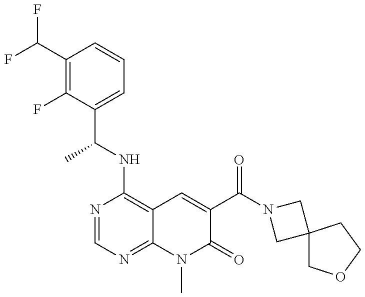 [SMILES: C[C@@H](Nc1ncnc2c1cc(C(=O)N1CC3(CCOC3)C1)c(=O)n2C)c1cccc(C(F)F)c1F]

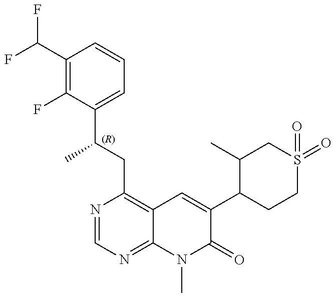

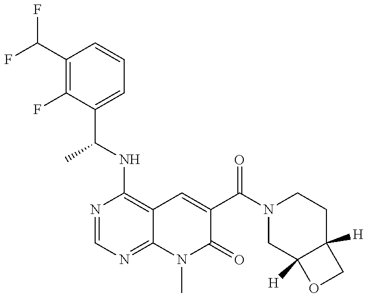 [SMILES: C[C@@H](Nc1ncnc2c1cc(C(=O)N1CC[C@@H]3CO[C@@H]3C1)c(=O)n2C)c1cccc(C(F)F)c1F]

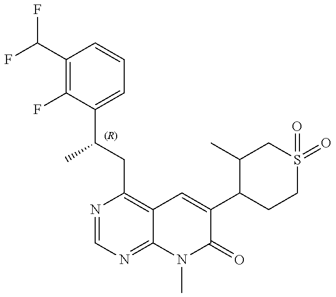

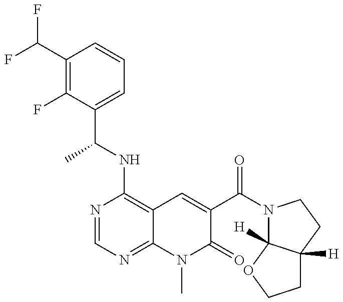 [SMILES: C[C@@H](Nc1ncnc2c1cc(C(=O)N1CC[C@@H]3CCO[C@@H]31)c(=O)n2C)c1cccc(C(F)F)c1F]

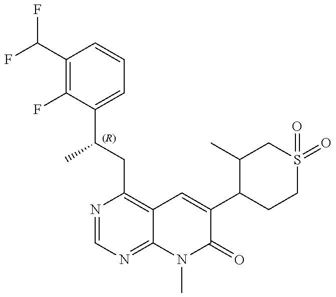

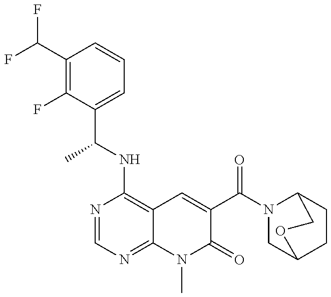 [SMILES: C[C@@H](Nc1ncnc2c1cc(C(=O)N1CC3CCC1CO3)c(=O)n2C)c1cccc(C(F)F)c1F]

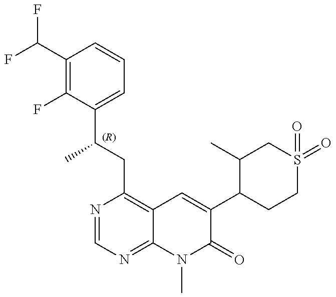

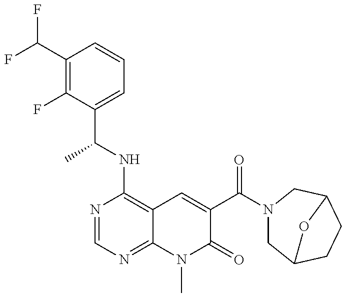 [SMILES: C[C@@H](Nc1ncnc2c1cc(C(=O)N1CC3CCC(C1)O3)c(=O)n2C)c1cccc(C(F)F)c1F]

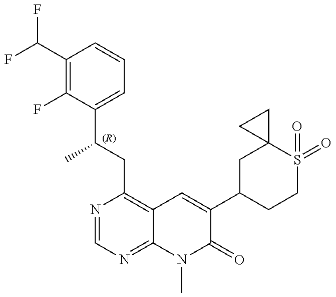

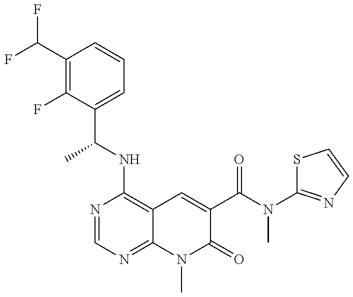 [SMILES: C[C@@H](Nc1ncnc2c1cc(C(=O)N(C)c1nccs1)c(=O)n2C)c1cccc(C(F)F)c1F]

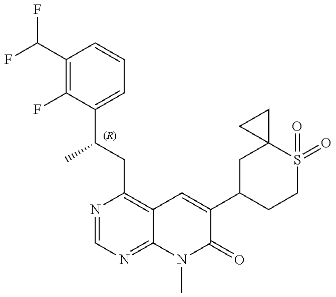

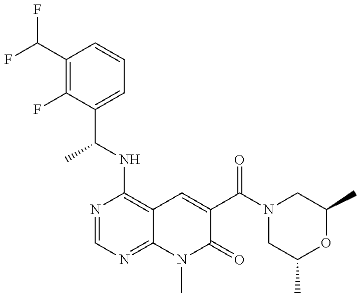 [SMILES: C[C@@H]1CN(C(=O)c2cc3c(N[C@H](C)c4cccc(C(F)F)c4F)ncnc3n(C)c2=O)C[C@@H](C)O1]

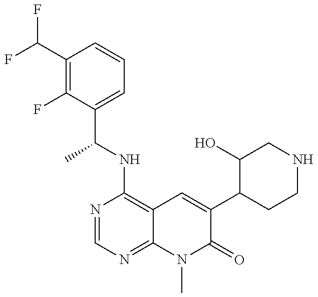 [SMILES: C[C@@H](Nc1ncnc2c1cc(C1CCNCC1O)c(=O)n2C)c1cccc(C(F)F)c1F]

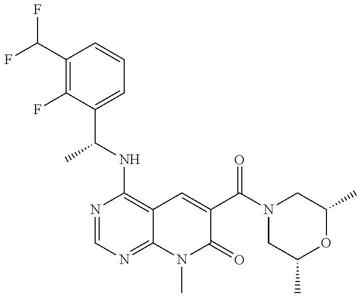 [SMILES: C[C@@H]1CN(C(=O)c2cc3c(N[C@H](C)c4cccc(C(F)F)c4F)ncnc3n(C)c2=O)C[C@H](C)O1]

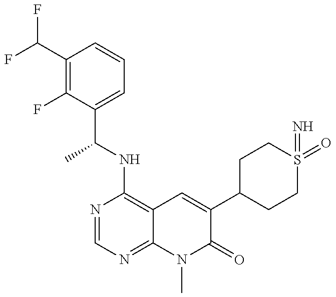 [SMILES: C[C@@H](Nc1ncnc2c1cc(C1CCS(=N)(=O)CC1)c(=O)n2C)c1cccc(C(F)F)c1F]

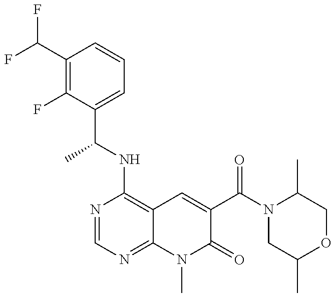 [SMILES: CC1CN(C(=O)c2cc3c(N[C@H](C)c4cccc(C(F)F)c4F)ncnc3n(C)c2=O)C(C)CO1]

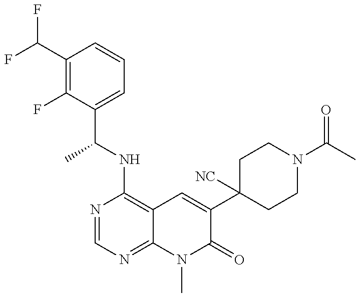 [SMILES: CC(=O)N1CCC(C#N)(c2cc3c(N[C@H](C)c4cccc(C(F)F)c4F)ncnc3n(C)c2=O)CC1]

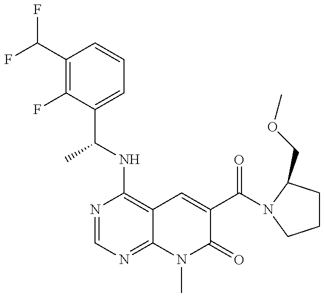 [SMILES: COC[C@H]1CCCN1C(=O)c1cc2c(N[C@H](C)c3cccc(C(F)F)c3F)ncnc2n(C)c1=O]

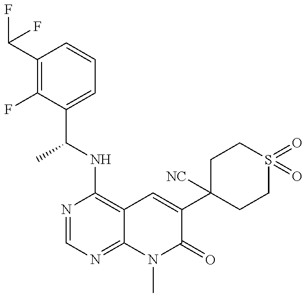 [SMILES: C[C@@H](Nc1ncnc2c1cc(C1(C#N)CCS(=O)(=O)CC1)c(=O)n2C)c1cccc(C(F)F)c1F]

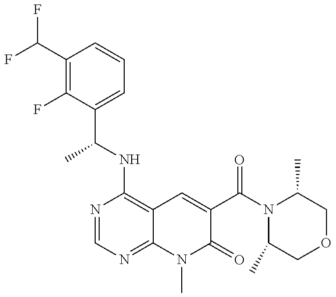 [SMILES: C[C@@H]1COC[C@H](C)N1C(=O)c1cc2c(N[C@H](C)c3cccc(C(F)F)c3F)ncnc2n(C)c1=O]

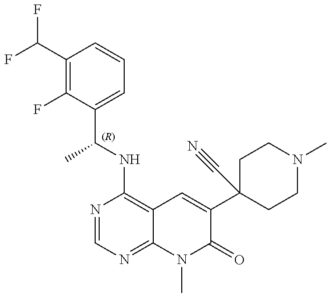

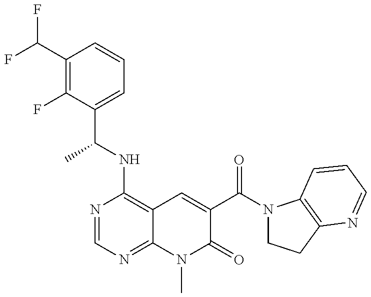 [SMILES: C[C@@H](Nc1ncnc2c1cc(C(=O)N1CCc3ncccc31)c(=O)n2C)c1cccc(C(F)F)c1F]

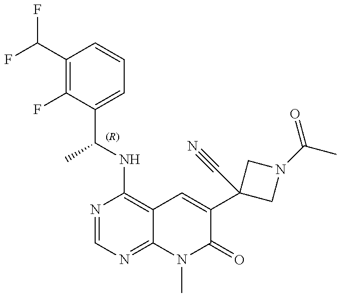

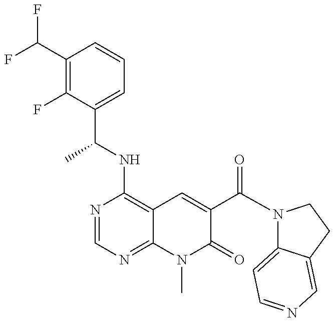 [SMILES: C[C@@H](Nc1ncnc2c1cc(C(=O)N1CCc3cnccc31)c(=O)n2C)c1cccc(C(F)F)c1F]

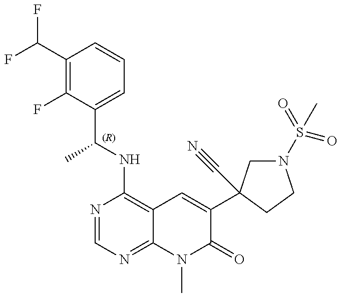

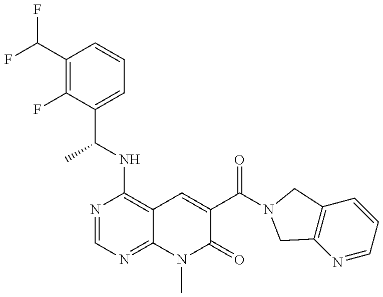 [SMILES: C[C@@H](Nc1ncnc2c1cc(C(=O)N1Cc3cccnc3C1)c(=O)n2C)c1cccc(C(F)F)c1F]

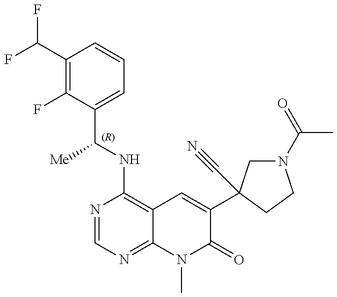

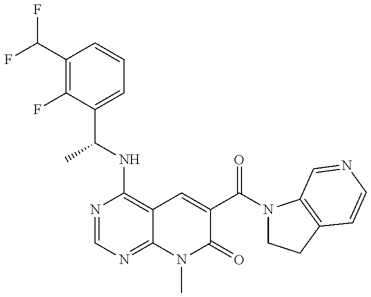 [SMILES: C[C@@H](Nc1ncnc2c1cc(C(=O)N1CCc3ccncc31)c(=O)n2C)c1cccc(C(F)F)c1F]

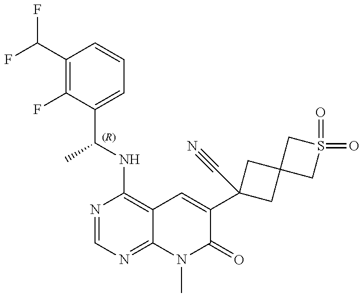

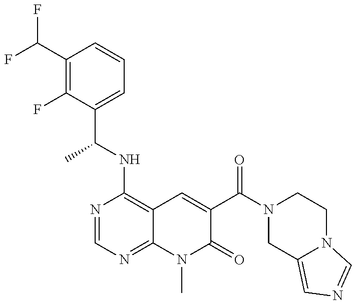 [SMILES: C[C@@H](Nc1ncnc2c1cc(C(=O)N1CCn3cncc3C1)c(=O)n2C)c1cccc(C(F)F)c1F]

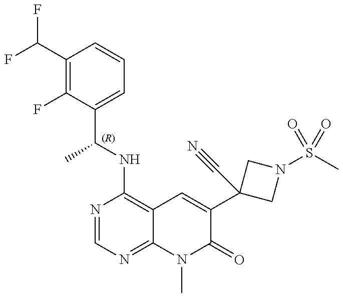

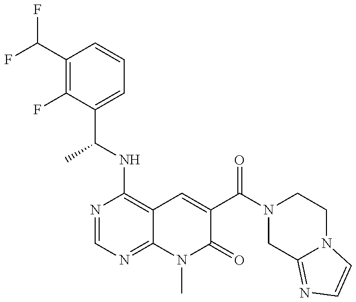 [SMILES: C[C@@H](Nc1ncnc2c1cc(C(=O)N1CCn3ccnc3C1)c(=O)n2C)c1cccc(C(F)F)c1F]

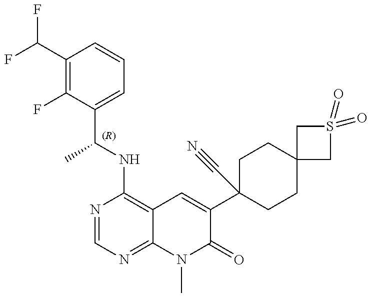

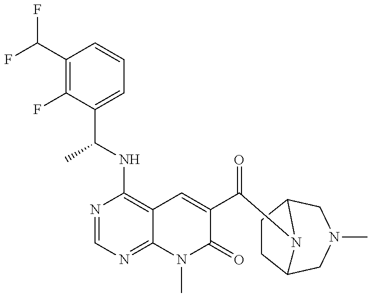 [SMILES: C[C@@H](Nc1ncnc2c1cc(C(=O)N1C3CCC1CN(C)C3)c(=O)n2C)c1cccc(C(F)F)c1F]

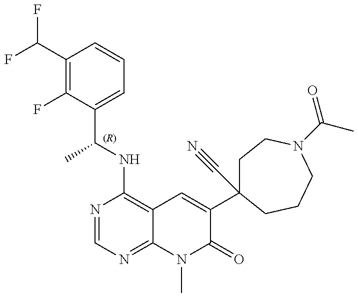

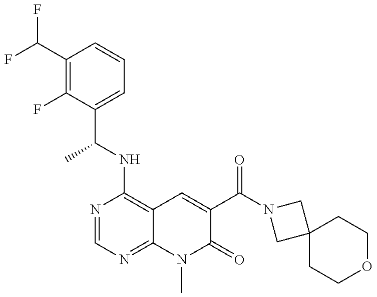 [SMILES: C[C@@H](Nc1ncnc2c1cc(C(=O)N1CC3(CCOCC3)C1)c(=O)n2C)c1cccc(C(F)F)c1F]

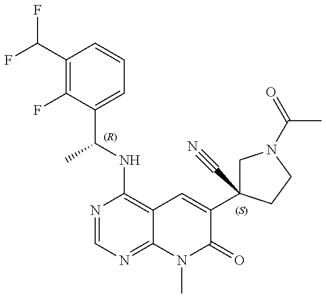

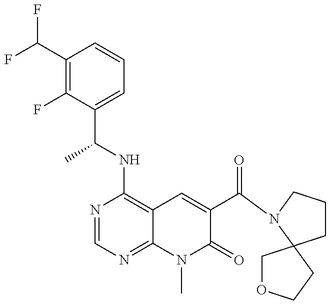 [SMILES: C[C@@H](Nc1ncnc2c1cc(C(=O)N1CCCC13CCOC3)c(=O)n2C)c1cccc(C(F)F)c1F]

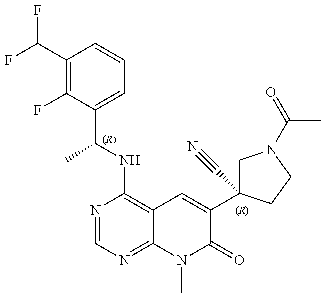

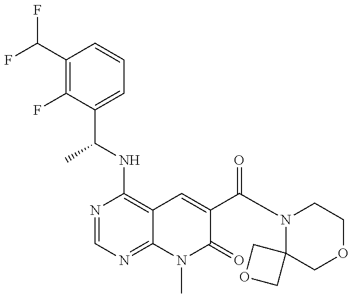 [SMILES: C[C@@H](Nc1ncnc2c1cc(C(=O)N1CCOCC13COC3)c(=O)n2C)c1cccc(C(F)F)c1F]

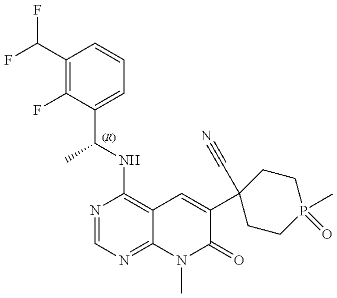

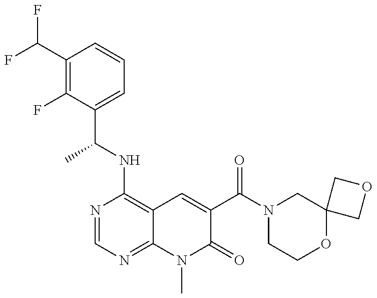 [SMILES: C[C@@H](Nc1ncnc2c1cc(C(=O)N1CCOC3(COC3)C1)c(=O)n2C)c1cccc(C(F)F)c1F]

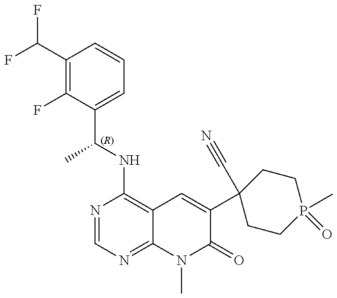

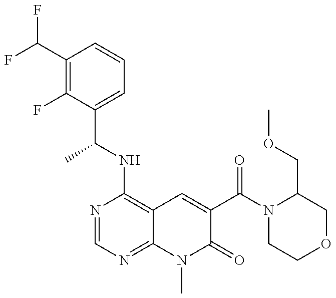 [SMILES: COCC1COCCN1C(=O)c1cc2c(N[C@H](C)c3cccc(C(F)F)c3F)ncnc2n(C)c1=O]

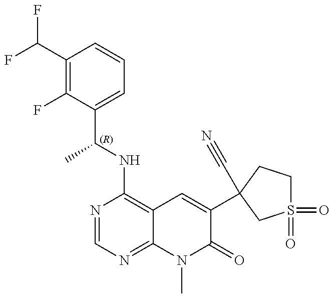

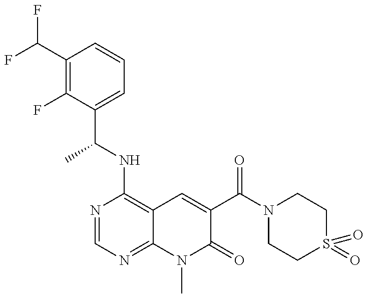 [SMILES: C[C@@H](Nc1ncnc2c1cc(C(=O)N1CCS(=O)(=O)CC1)c(=O)n2C)c1cccc(C(F)F)c1F]

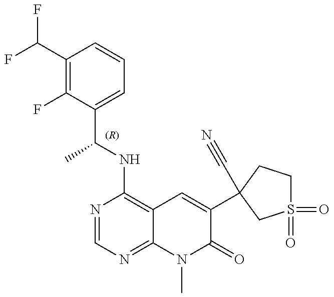

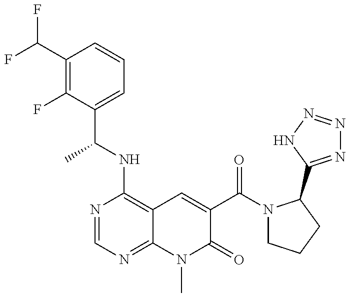 [SMILES: C[C@@H](Nc1ncnc2c1cc(C(=O)N1CCC[C@@H]1c1nnn[nH]1)c(=O)n2C)c1cccc(C(F)F)c1F]

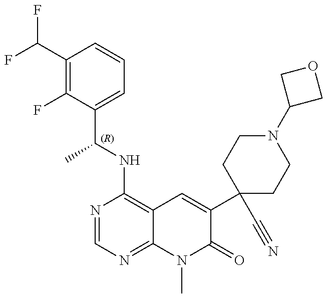

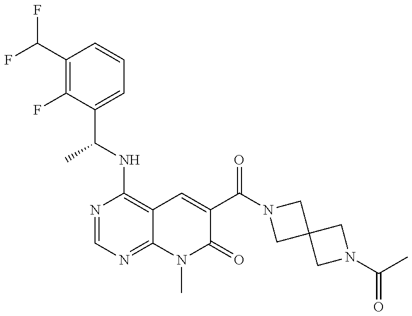 [SMILES: CC(=O)N1CC2(C1)CN(C(=O)c1cc3c(N[C@H](C)c4cccc(C(F)F)c4F)ncnc3n(C)c1=O)C2]

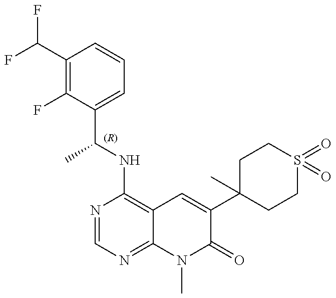

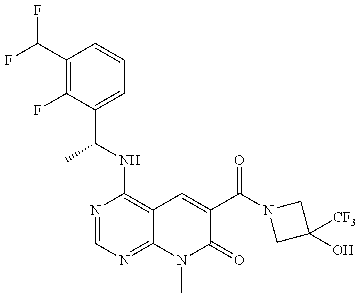 [SMILES: C[C@@H](Nc1ncnc2c1cc(C(=O)N1CC(O)(C(F)(F)F)C1)c(=O)n2C)c1cccc(C(F)F)c1F]

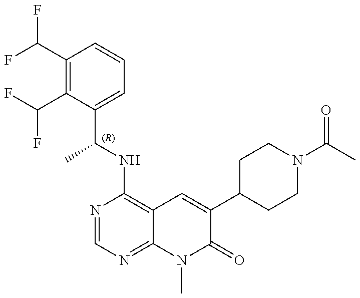

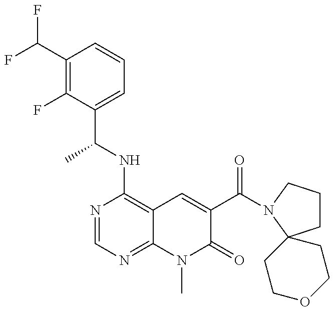 [SMILES: C[C@@H](Nc1ncnc2c1cc(C(=O)N1CCCC13CCOCC3)c(=O)n2C)c1cccc(C(F)F)c1F]

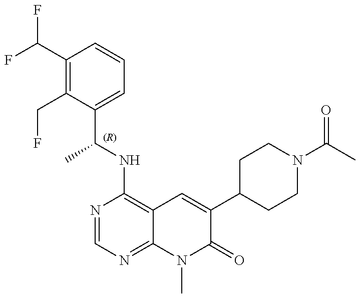

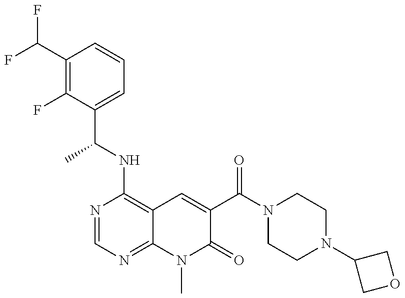 [SMILES: C[C@@H](Nc1ncnc2c1cc(C(=O)N1CCN(C3COC3)CC1)c(=O)n2C)c1cccc(C(F)F)c1F]

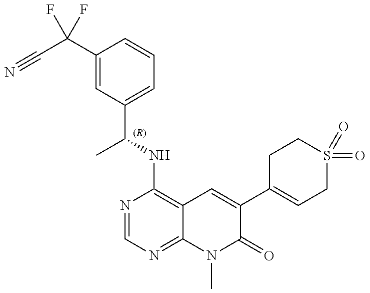

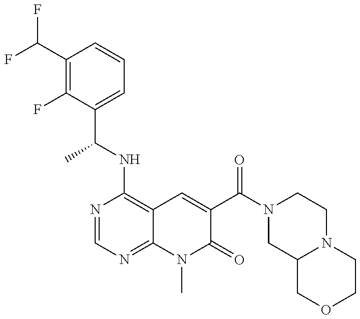 [SMILES: C[C@@H](Nc1ncnc2c1cc(C(=O)N1CCN3CCOCC3C1)c(=O)n2C)c1cccc(C(F)F)c1F]

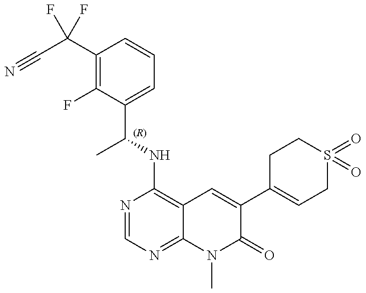

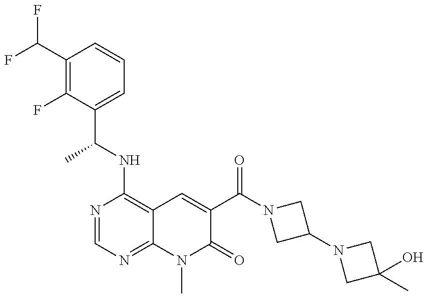 [SMILES: C[C@@H](Nc1ncnc2c1cc(C(=O)N1CC(N3CC(C)(O)C3)C1)c(=O)n2C)c1cccc(C(F)F)c1F]

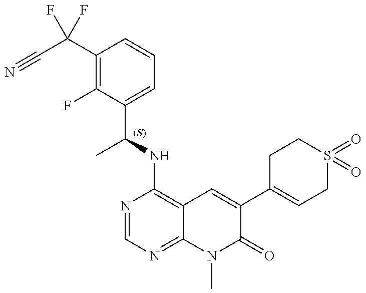

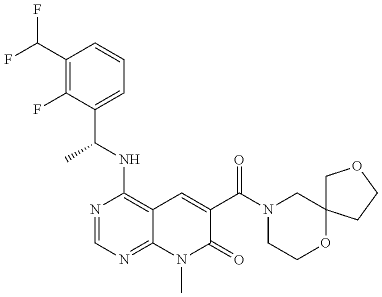 [SMILES: C[C@@H](Nc1ncnc2c1cc(C(=O)N1CCOC3(CCOC3)C1)c(=O)n2C)c1cccc(C(F)F)c1F]

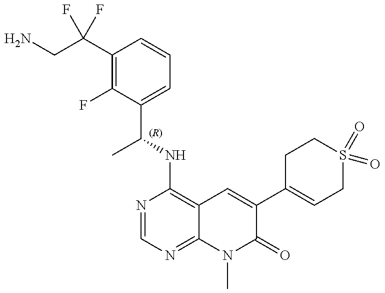

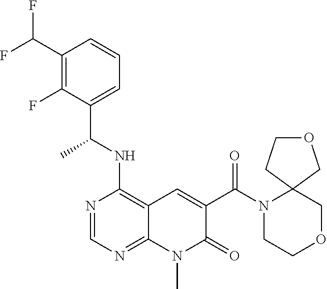 [SMILES: C[C@@H](Nc1ncnc2c1cc(C(=O)N1CCOCC13CCOC3)c(=O)n2C)c1cccc(C(F)F)c1F]

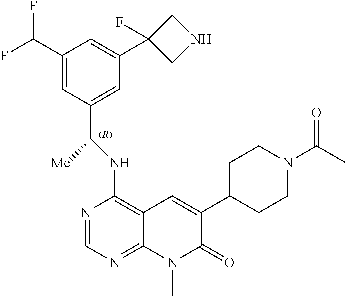

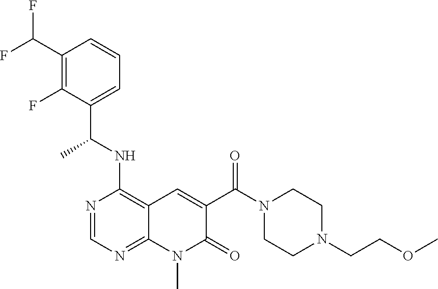 [SMILES: COCCN1CCN(C(=O)c2cc3c(N[C@H](C)c4cccc(C(F)F)c4F)ncnc3n(C)c2=O)CC1]

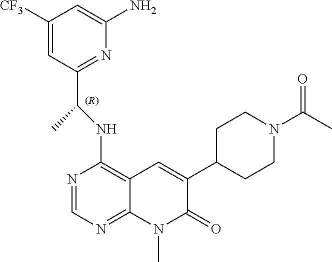

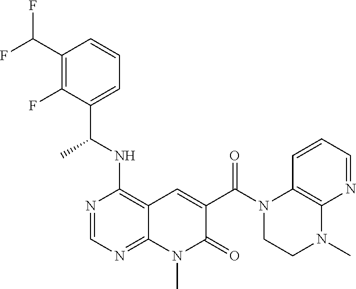 [SMILES: C[C@@H](Nc1ncnc2c1cc(C(=O)N1CCN(C)c3ncccc31)c(=O)n2C)c1cccc(C(F)F)c1F]

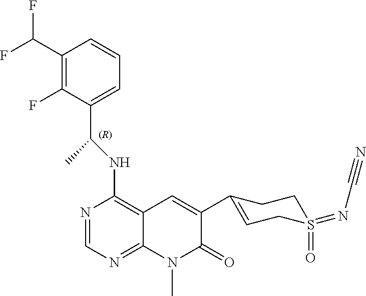

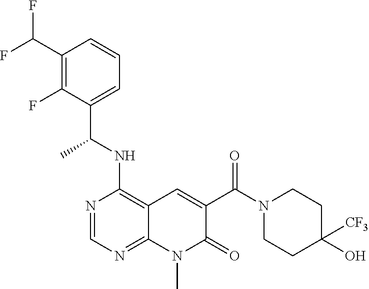 [SMILES: C[C@@H](Nc1ncnc2c1cc(C(=O)N1CCC(O)(C(F)(F)F)CC1)c(=O)n2C)c1cccc(C(F)F)c1F]

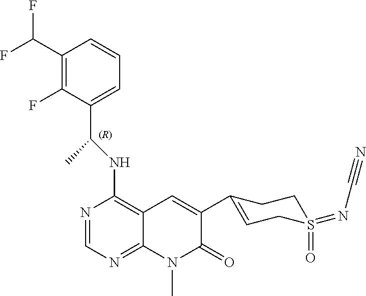

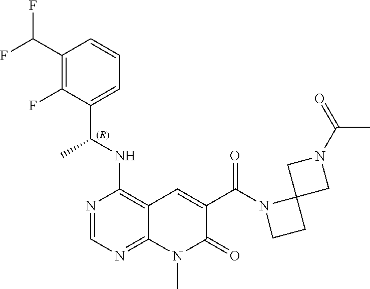

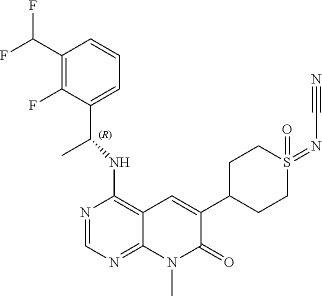

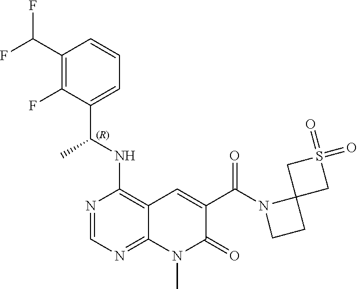

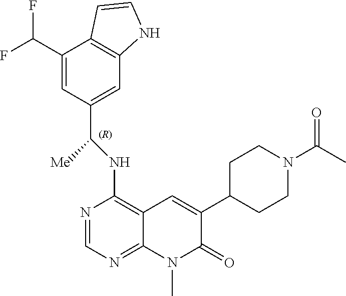

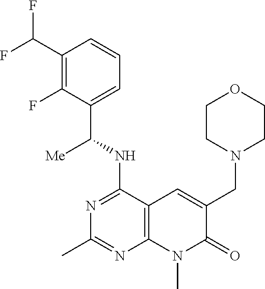 [SMILES: Cc1nc(N[C@H](C)c2cccc(C(F)F)c2F)c2cc(CN3CCOCC3)c(=O)n(C)c2n1]

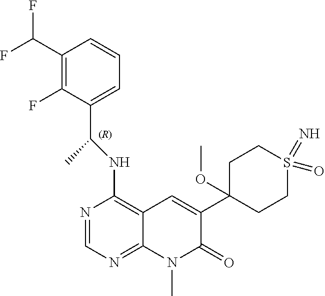

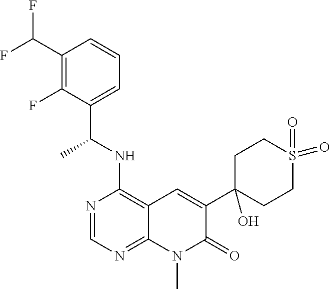 [SMILES: C[C@@H](Nc1ncnc2c1cc(C1(O)CCS(=O)(=O)CC1)c(=O)n2C)c1cccc(C(F)F)c1F]

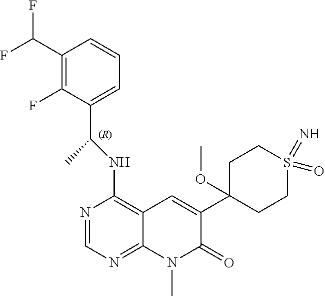

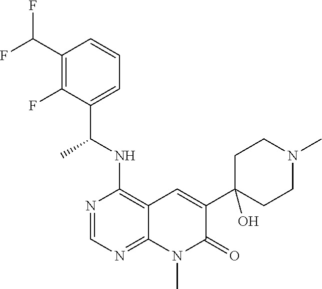 [SMILES: C[C@@H](Nc1ncnc2c1cc(C1(O)CCN(C)CC1)c(=O)n2C)c1cccc(C(F)F)c1F]

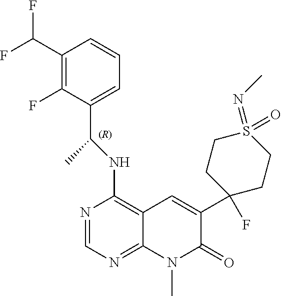

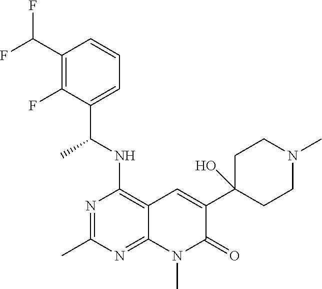 [SMILES: Cc1nc(N[C@H](C)c2cccc(C(F)F)c2F)c2cc(C3(O)CCN(C)CC3)c(=O)n(C)c2n1]

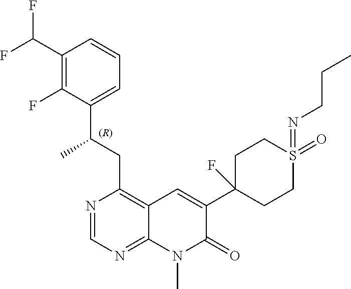

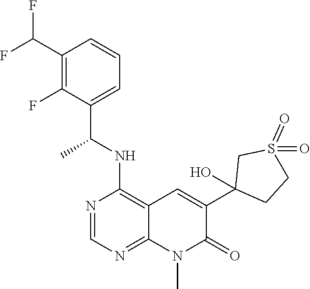 [SMILES: C[C@@H](Nc1ncnc2c1cc(C1(O)CCS(=O)(=O)C1)c(=O)n2C)c1cccc(C(F)F)c1F]

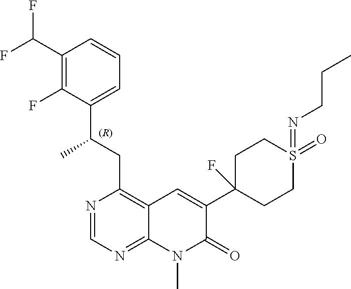

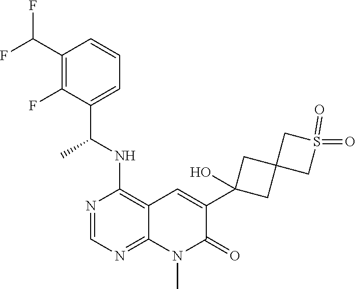 [SMILES: C[C@@H](Nc1ncnc2c1cc(C1(O)CC3(C1)CS(=O)(=O)C3)c(=O)n2C)c1cccc(C(F)F)c1F]

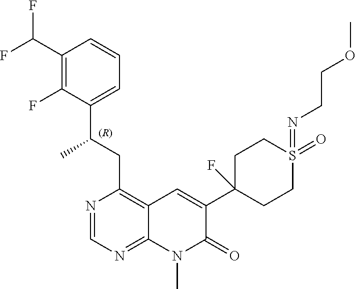

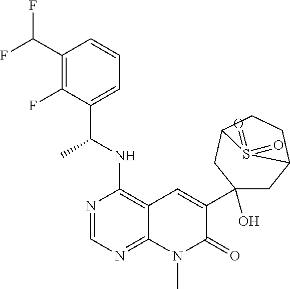 [SMILES: C[C@@H](Nc1ncnc2c1cc(C1(O)CC3CCC(C1)S3(=O)=O)c(=O)n2C)c1cccc(C(F)F)c1F]

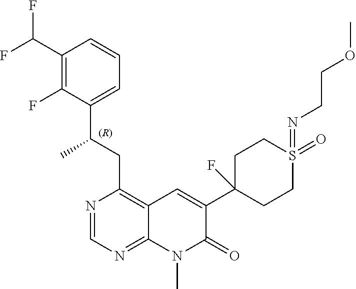

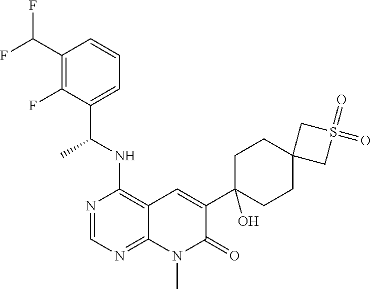 [SMILES: C[C@@H](Nc1ncnc2c1cc(C1(O)CCC3(CC1)CS(=O)(=O)C3)c(=O)n2C)c1cccc(C(F)F)c1F]

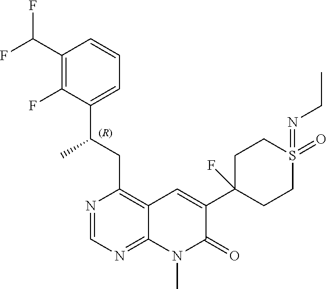

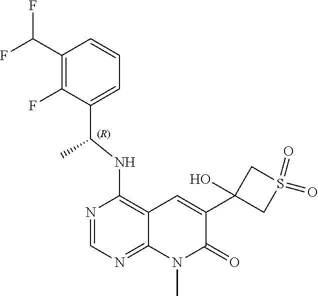

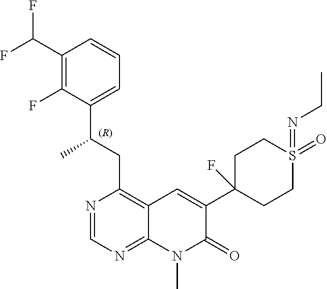

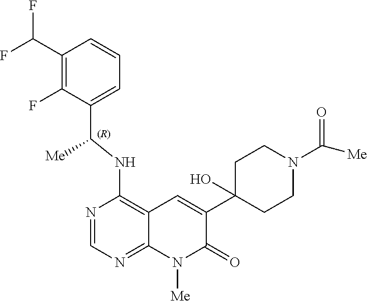

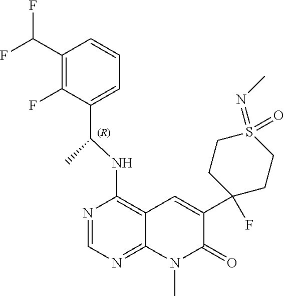

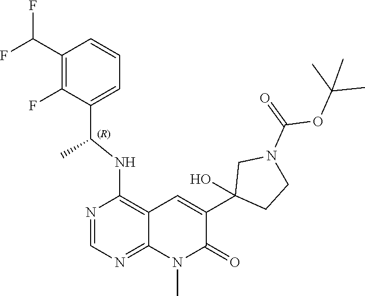

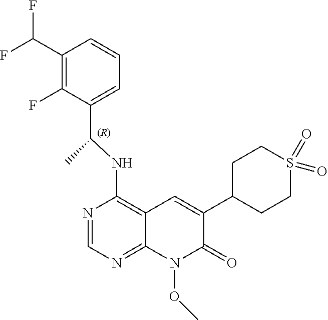

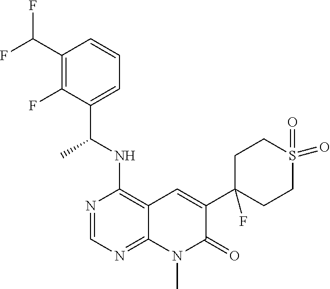 [SMILES: C[C@@H](Nc1ncnc2c1cc(C1(F)CCS(=O)(=O)CC1)c(=O)n2C)c1cccc(C(F)F)c1F]

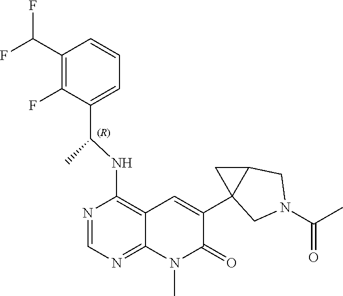

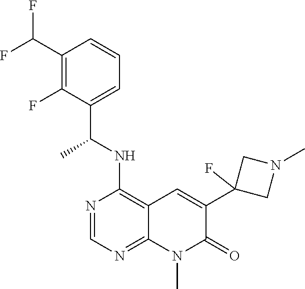 [SMILES: C[C@@H](Nc1ncnc2c1cc(C1(F)CN(C)C1)c(=O)n2C)c1cccc(C(F)F)c1F]

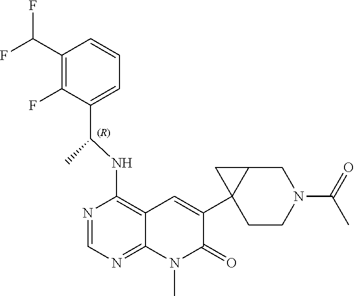

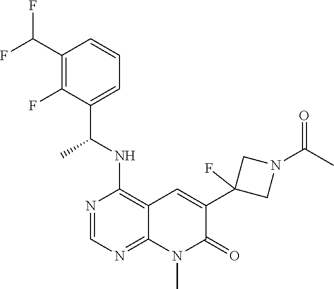 [SMILES: CC(=O)N1CC(F)(c2cc3c(N[C@H](C)c4cccc(C(F)F)c4F)ncnc3n(C)c2=O)C1]

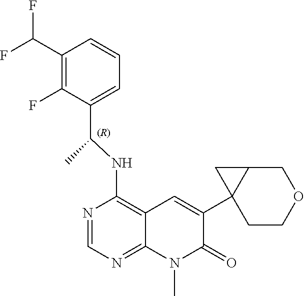

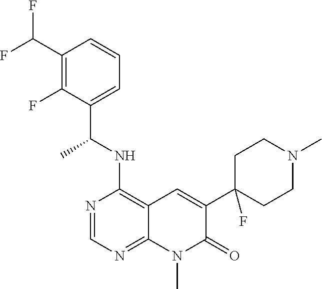 [SMILES: C[C@@H](Nc1ncnc2c1cc(C1(F)CCN(C)CC1)c(=O)n2C)c1cccc(C(F)F)c1F]

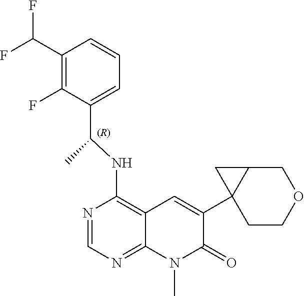

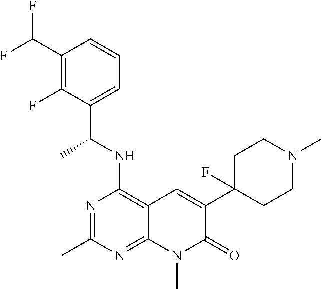 [SMILES: Cc1nc(N[C@H](C)c2cccc(C(F)F)c2F)c2cc(C3(F)CCN(C)CC3)c(=O)n(C)c2n1]

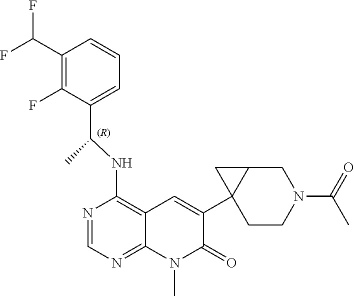

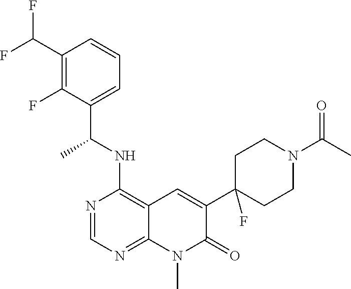 [SMILES: CC(=O)N1CCC(F)(c2cc3c(N[C@H](C)c4cccc(C(F)F)c4F)ncnc3n(C)c2=O)CC1]

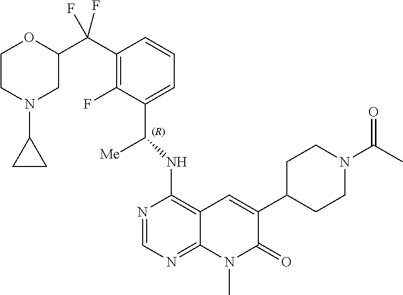

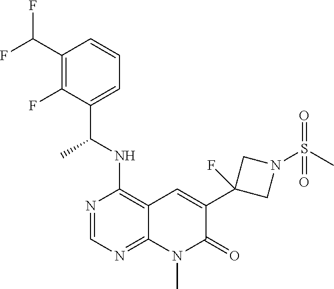 [SMILES: C[C@@H](Nc1ncnc2c1cc(C1(F)CN(S(C)(=O)=O)C1)c(=O)n2C)c1cccc(C(F)F)c1F]

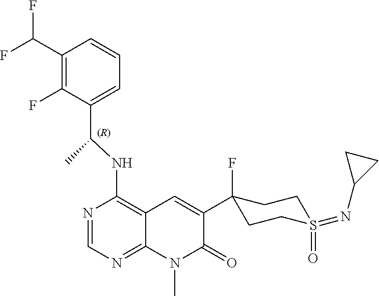

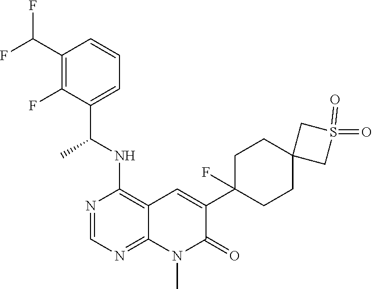 [SMILES: C[C@@H](Nc1ncnc2c1cc(C1(F)CCC3(CC1)CS(=O)(=O)C3)c(=O)n2C)c1cccc(C(F)F)c1F]

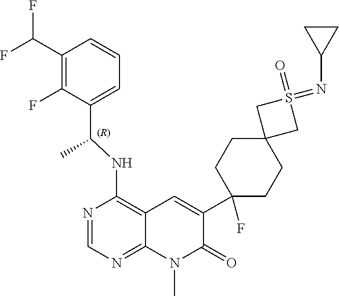

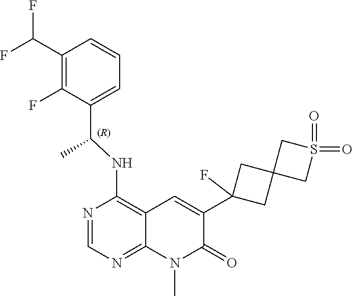

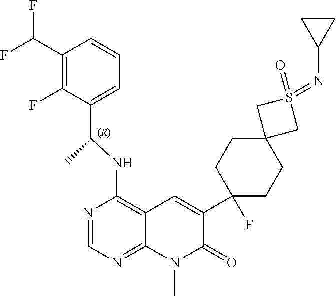

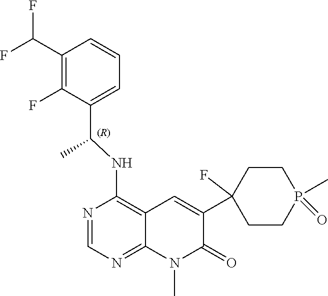

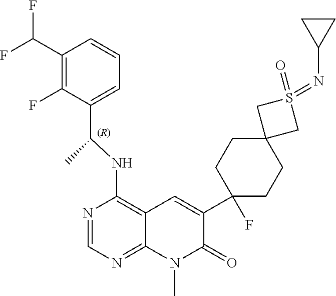

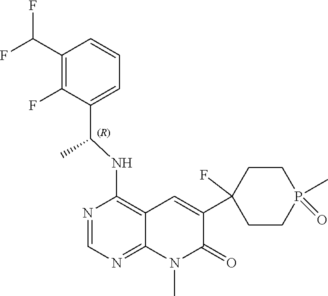

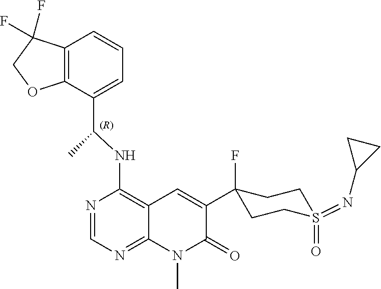

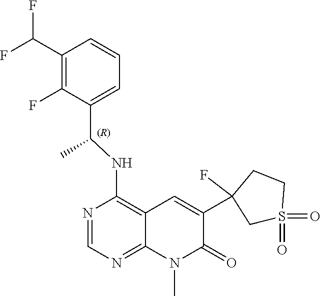

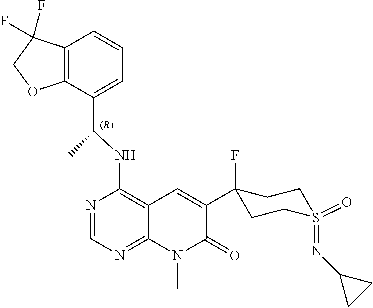

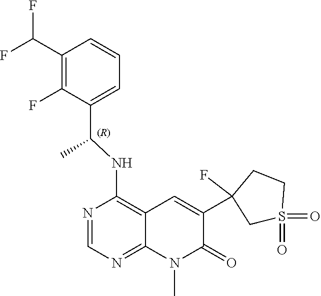

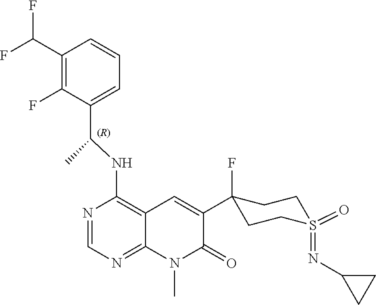

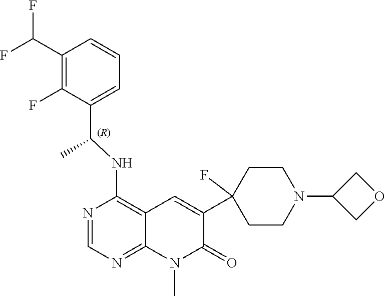

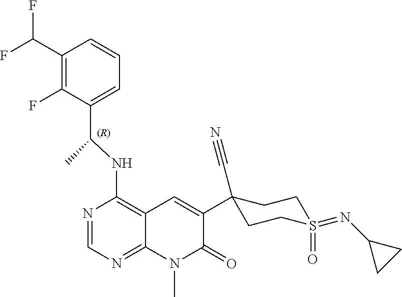

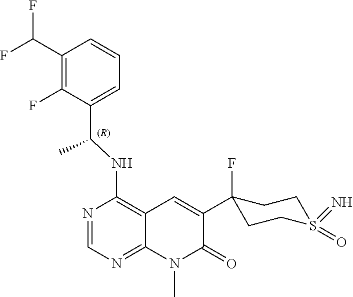

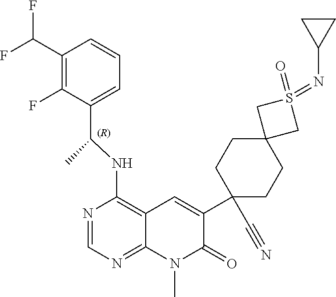

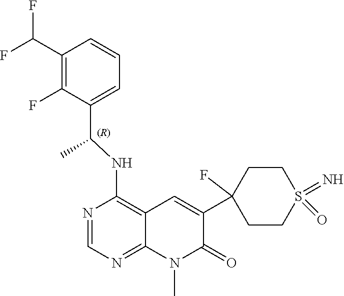

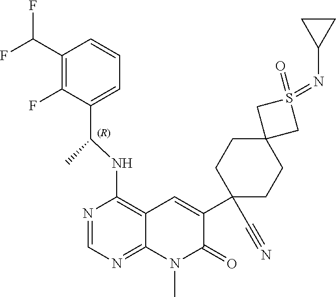

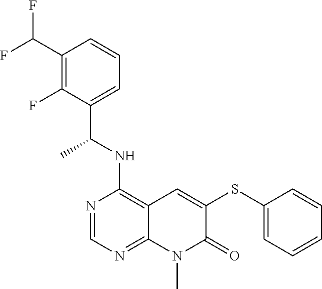 [SMILES: C[C@@H](Nc1ncnc2c1cc(Sc1ccccc1)c(=O)n2C)c1cccc(C(F)F)c1F]

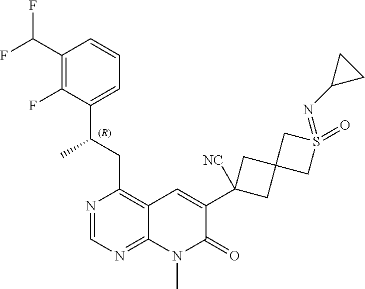

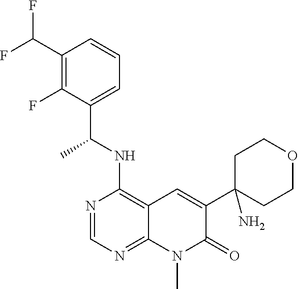 [SMILES: C[C@@H](Nc1ncnc2c1cc(C1(N)CCOCC1)c(=O)n2C)c1cccc(C(F)F)c1F]

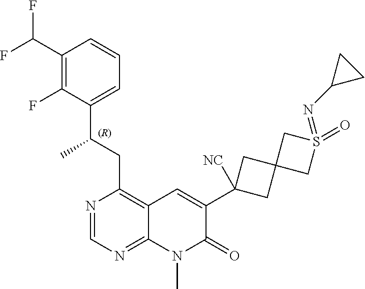

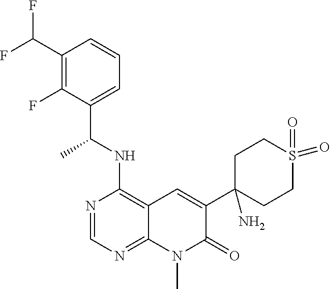 [SMILES: C[C@@H](Nc1ncnc2c1cc(C1(N)CCS(=O)(=O)CC1)c(=O)n2C)c1cccc(C(F)F)c1F]

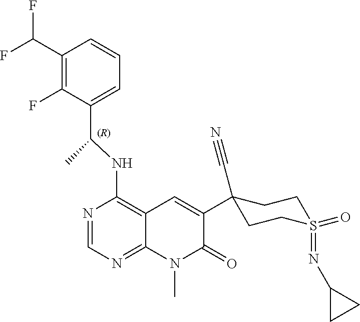

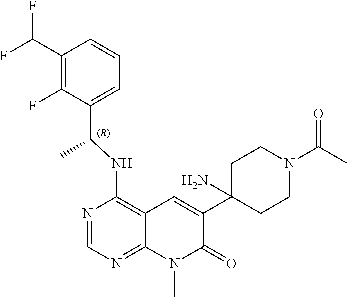

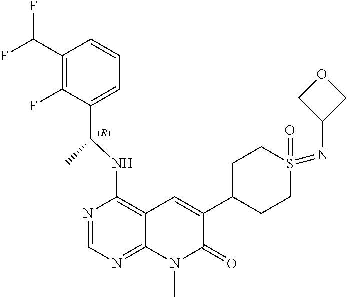

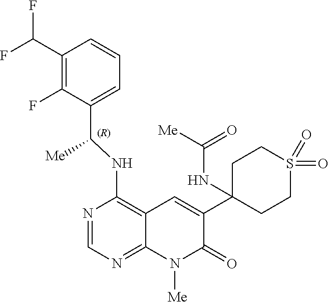

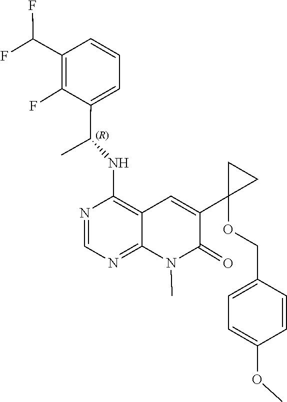

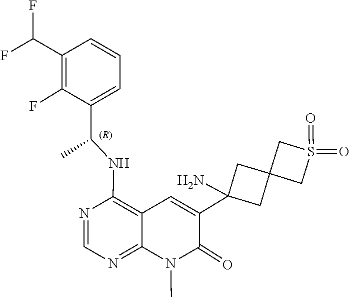

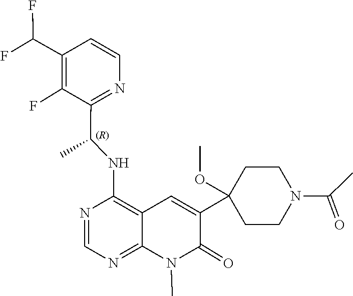

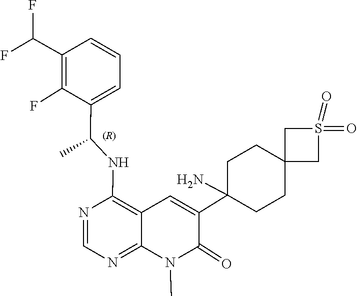

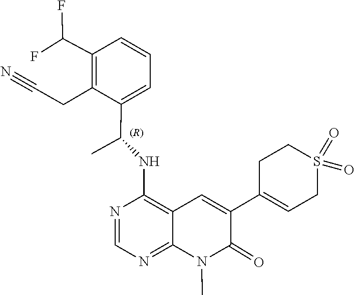

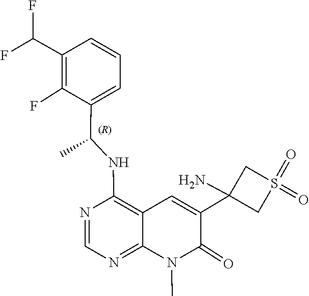

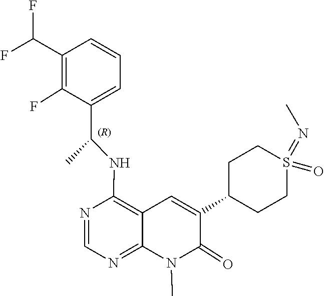

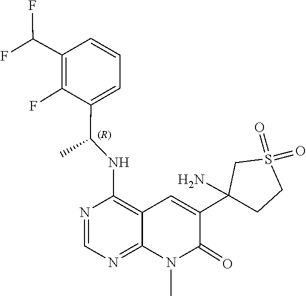

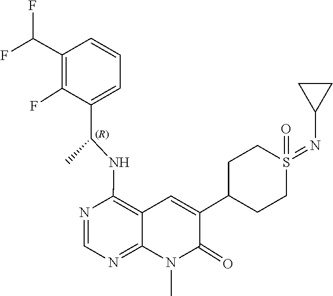

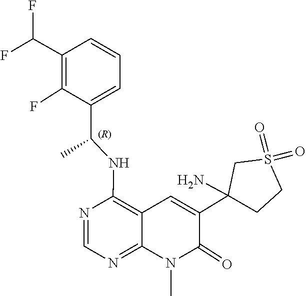

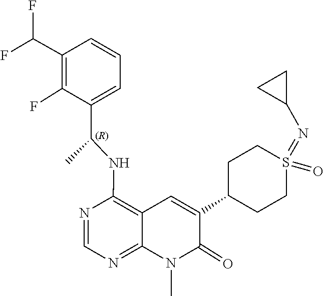

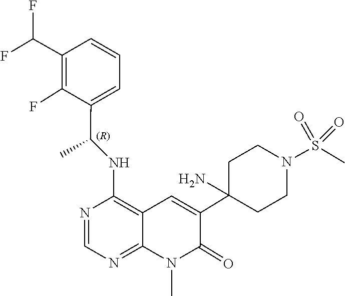

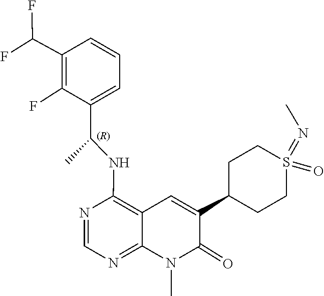

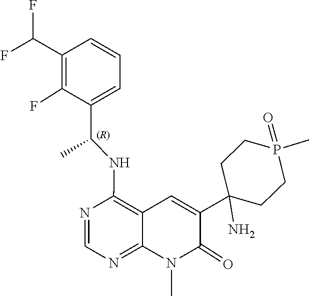

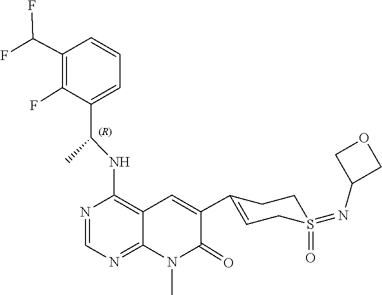

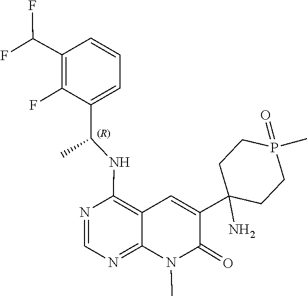

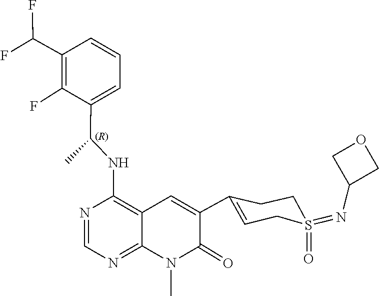

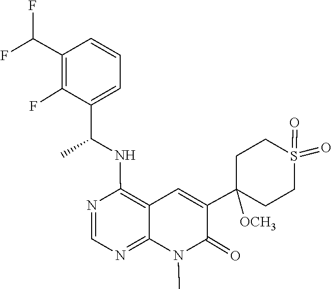 [SMILES: COC1(c2cc3c(N[C@H](C)c4cccc(C(F)F)c4F)ncnc3n(C)c2=O)CCS(=O)(=O)CC1]

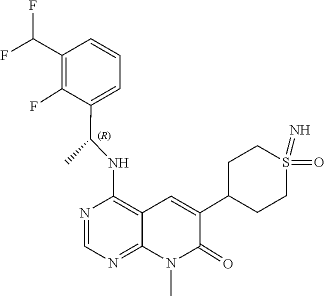

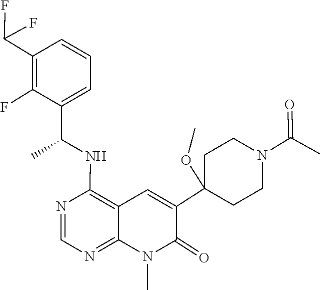 [SMILES: COC1(c2cc3c(N[C@H](C)c4cccc(C(F)F)c4F)ncnc3n(C)c2=O)CCN(C(C)=O)CC1]

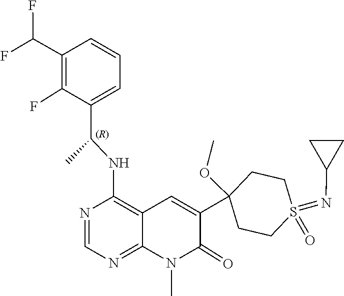

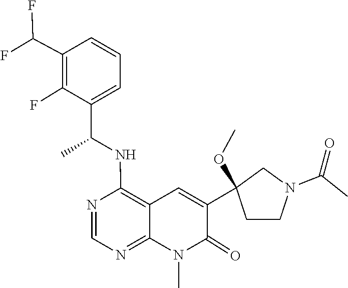 [SMILES: CO[C@]1(c2cc3c(N[C@H](C)c4cccc(C(F)F)c4F)ncnc3n(C)c2=O)CCN(C(C)=O)C1]

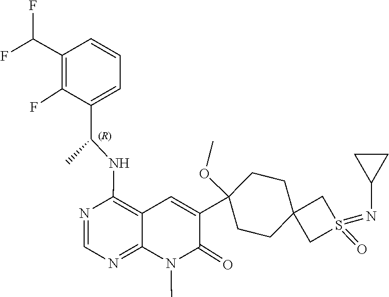

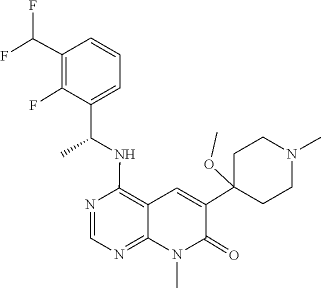 [SMILES: COC1(c2cc3c(N[C@H](C)c4cccc(C(F)F)c4F)ncnc3n(C)c2=O)CCN(C)CC1]

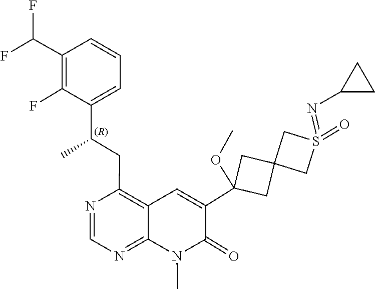

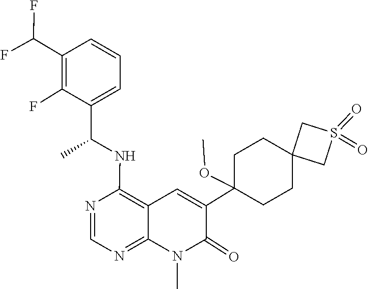 [SMILES: COC1(c2cc3c(N[C@H](C)c4cccc(C(F)F)c4F)ncnc3n(C)c2=O)CCC2(CC1)CS(=O)(=O)C2]

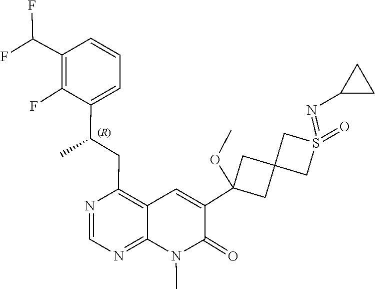

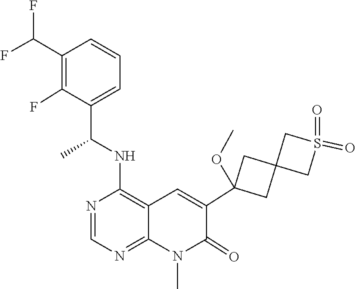 [SMILES: COC1(c2cc3c(N[C@H](C)c4cccc(C(F)F)c4F)ncnc3n(C)c2=O)CC2(C1)CS(=O)(=O)C2]

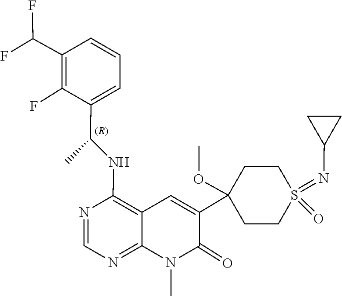

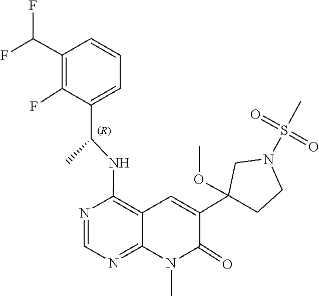

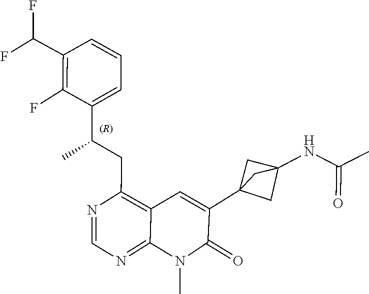

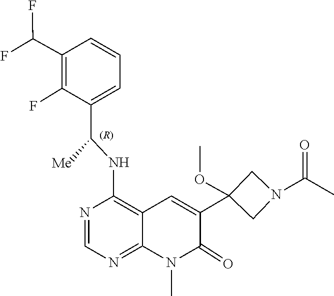

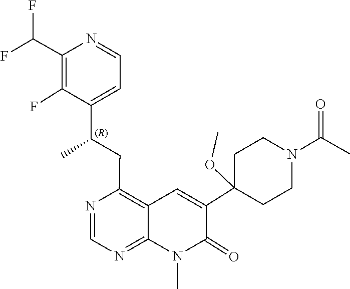

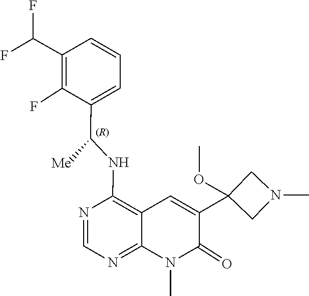

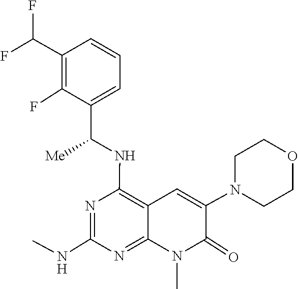 [SMILES: CNc1nc(N[C@H](C)c2cccc(C(F)F)c2F)c2cc(N3CCOCC3)c(=O)n(C)c2n1]

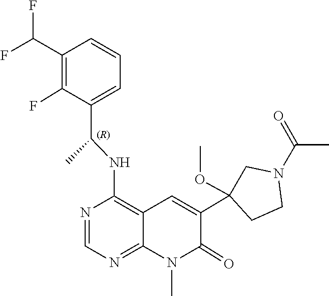

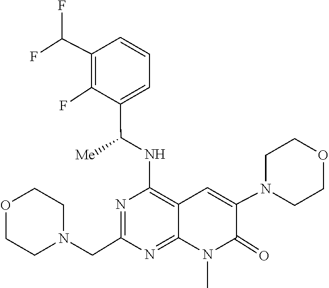 [SMILES: C[C@@H](Nc1nc(CN2CCOCC2)nc2c1cc(N1CCOCC1)c(=O)n2C)c1cccc(C(F)F)c1F]

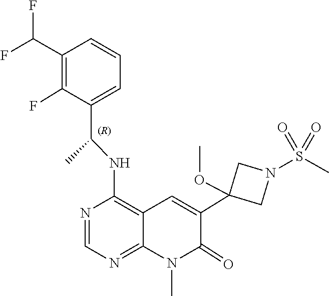

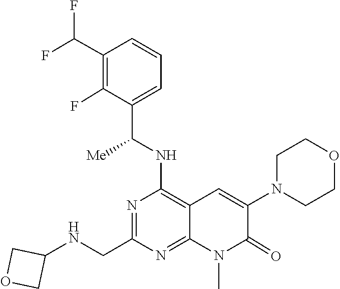 [SMILES: C[C@@H](Nc1nc(CNC2COC2)nc2c1cc(N1CCOCC1)c(=O)n2C)c1cccc(C(F)F)c1F]

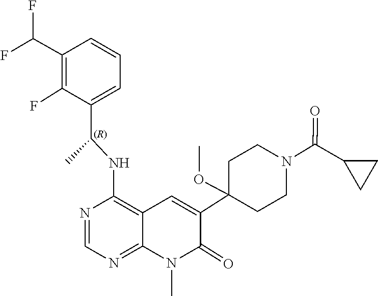

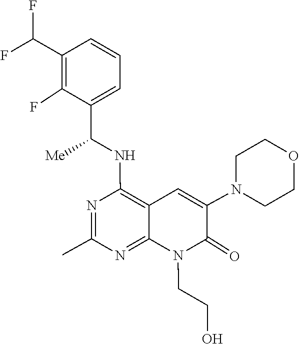 [SMILES: Cc1nc(N[C@H](C)c2cccc(C(F)F)c2F)c2cc(N3CCOCC3)c(=O)n(CCO)c2n1]

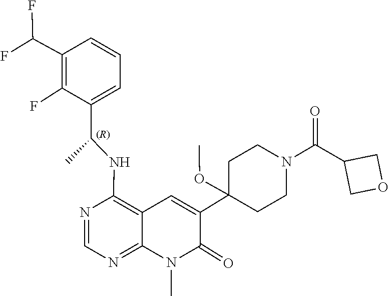

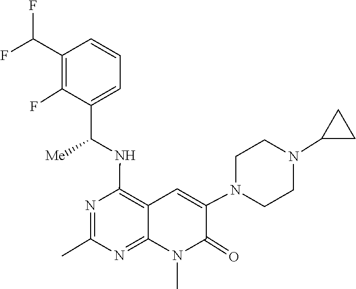 [SMILES: Cc1nc(N[C@H](C)c2cccc(C(F)F)c2F)c2cc(N3CCN(C4CC4)CC3)c(=O)n(C)c2n1]

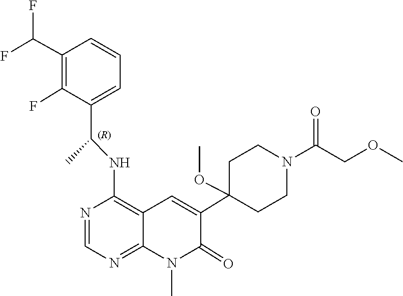

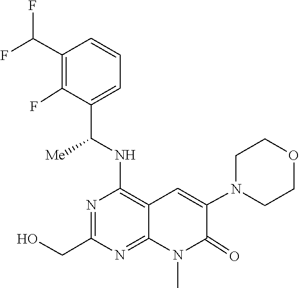 [SMILES: C[C@@H](Nc1nc(CO)nc2c1cc(N1CCOCC1)c(=O)n2C)c1cccc(C(F)F)c1F]

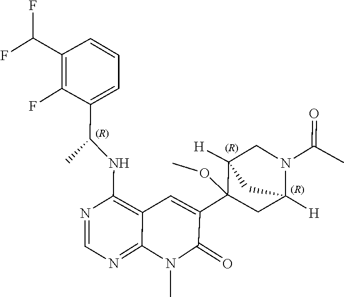

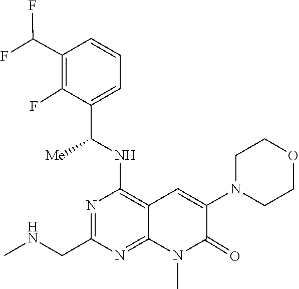 [SMILES: CNCc1nc(N[C@H](C)c2cccc(C(F)F)c2F)c2cc(N3CCOCC3)c(=O)n(C)c2n1]

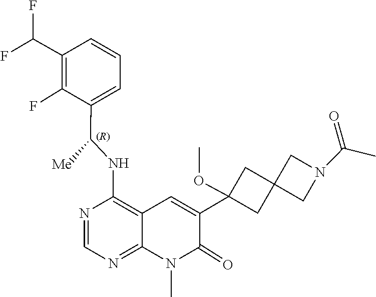

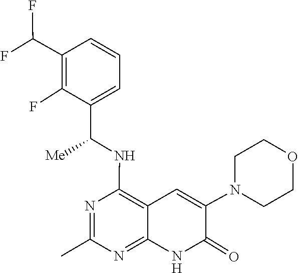 [SMILES: Cc1nc(N[C@H](C)c2cccc(C(F)F)c2F)c2cc(N3CCOCC3)c(=O)[nH]c2n1]

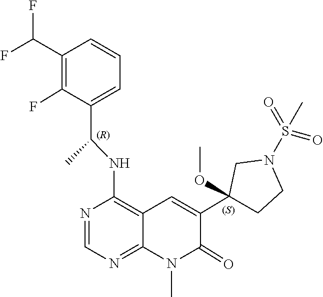

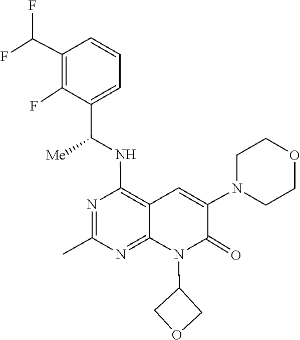 [SMILES: Cc1nc(N[C@H](C)c2cccc(C(F)F)c2F)c2cc(N3CCOCC3)c(=O)n(C3COC3)c2n1]

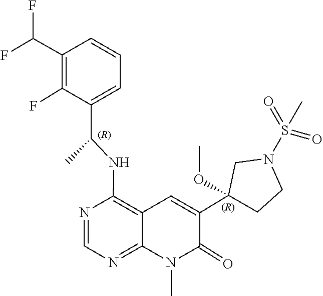

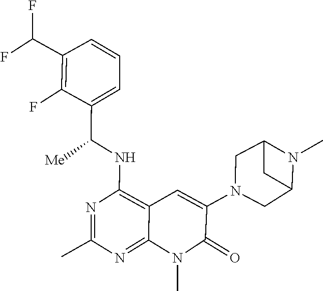 [SMILES: Cc1nc(N[C@H](C)c2cccc(C(F)F)c2F)c2cc(N3CC4CC(C3)N4C)c(=O)n(C)c2n1]

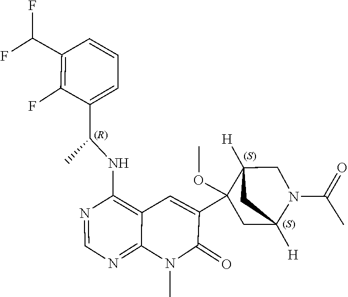

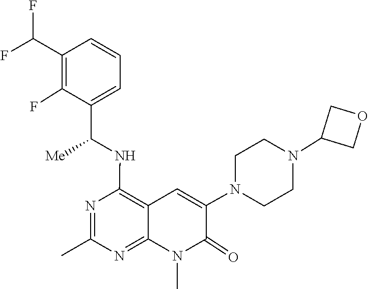 [SMILES: Cc1nc(N[C@H](C)c2cccc(C(F)F)c2F)c2cc(N3CCN(C4COC4)CC3)c(=O)n(C)c2n1]

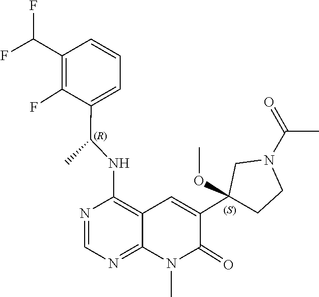

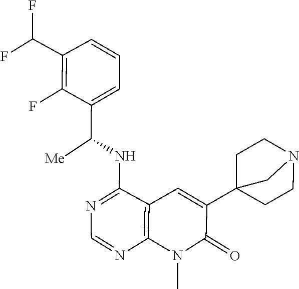 [SMILES: C[C@@H](Nc1ncnc2c1cc(C13CCN(CC1)C3)c(=O)n2C)c1cccc(C(F)F)c1F]

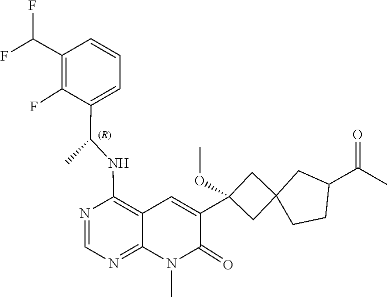

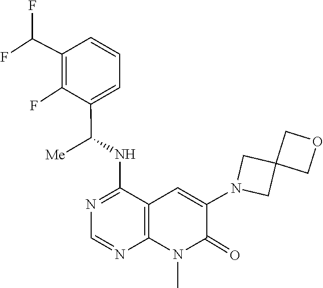 [SMILES: C[C@@H](Nc1ncnc2c1cc(N1CC3(COC3)C1)c(=O)n2C)c1cccc(C(F)F)c1F]

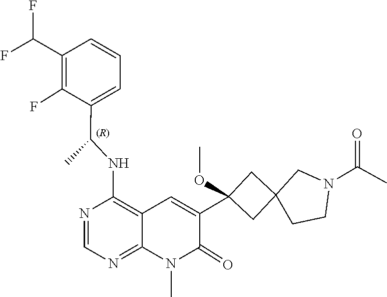

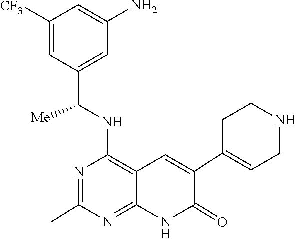 [SMILES: Cc1nc(N[C@H](C)c2cc(N)cc(C(F)(F)F)c2)c2cc(C3=CCNCC3)c(=O)[nH]c2n1]

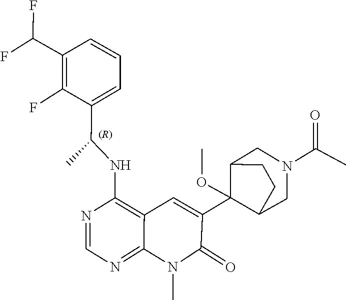

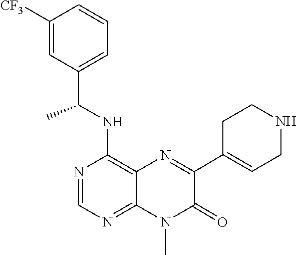 [SMILES: C[C@@H](Nc1ncnc2c1nc(C1=CCNCC1)c(=O)n2C)c1cccc(C(F)(F)F)c1]

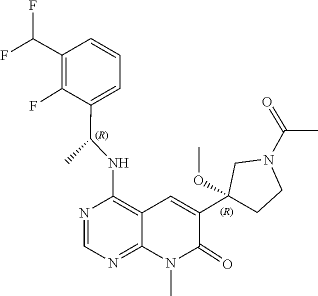

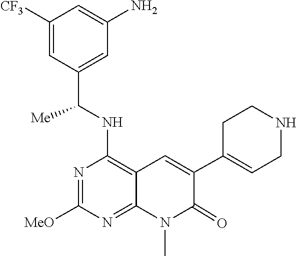 [SMILES: COc1nc(N[C@H](C)c2cc(N)cc(C(F)(F)F)c2)c2cc(C3=CCNCC3)c(=O)n(C)c2n1]

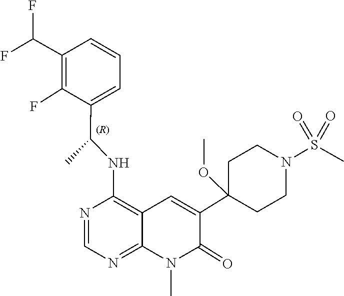

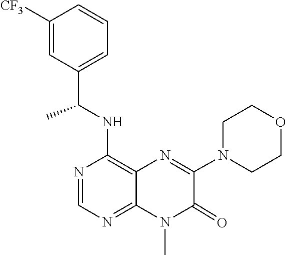 [SMILES: C[C@@H](Nc1ncnc2c1nc(N1CCOCC1)c(=O)n2C)c1cccc(C(F)(F)F)c1]

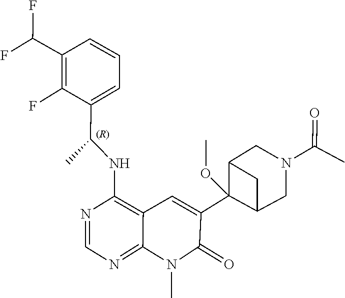

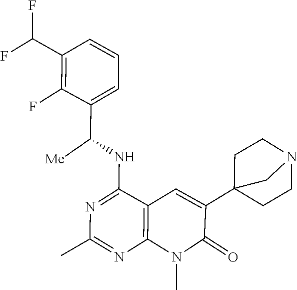 [SMILES: Cc1nc(N[C@H](C)c2cccc(C(F)F)c2F)c2cc(C34CCN(CC3)C4)c(=O)n(C)c2n1]

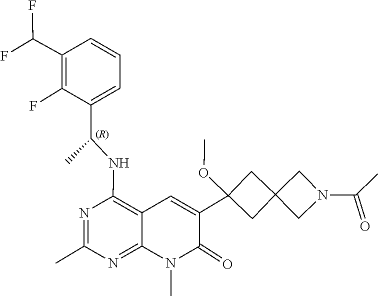

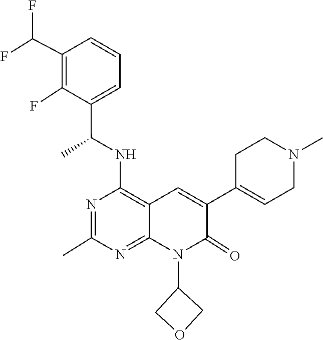 [SMILES: Cc1nc(N[C@H](C)c2cccc(C(F)F)c2F)c2cc(C3=CCN(C)CC3)c(=O)n(C3COC3)c2n1]

Note:
any compound shown in the foregoing table may be a diastereomer, and the
absolute stereochemistry of such diastereomer may not be known.

In some embodiments, the SOS1 inhibitor is selected from the group consisting of:
(R)-4-((1-(3-amino-5-(trifluoromethyl)phenyl)ethyl)amino)-8-methyl-6-(1,2,3,6-tetrahydropyridin-4-yl)pyrido[2,3-d]pyrimidin-7(8H)-one;
(R)-4-((1-(3-(1,1-difluoro-2-hydroxyethyl)phenyl)ethyl)amino)-8-methyl-6-morpholinopyrido[2,3-d]pyrimidin-7(8H)-one;
(R)-6-(3,6-dihydro-2H-pyran-4-yl)-8-methyl-4-((1-(3-(trifluoromethyl)phenyl)ethyl)amino)pyrido[2,3-d]pyrimidin-7(8H)-one;
4-{[(1R)-1-[3-amino-5-(trifluoromethyl)phenyl]ethyl]amino}-8-methyl-6-(morpholin-4-yl)-7H,8H-pyrido[2,3-d]pyrimidin-7-one;
8-methyl-6-(morpholin-4-yl)-4-{[(1R)-1-[3-(trifluoromethyl)phenyl]-ethyl]amino}-7H,8H-pyrido[2,3-d]pyrimidin-7-one;
4-{[(1R)-1-[3-(difluoromethyl)-2-fluorophenyl]ethyl]amino}-6-(1-methanesulfonyl-3-methylazetidin-3-yl)-8-methyl-7H,8H-pyrido[2,3-d]pyrimidin-7-one;
6-(1-acetyl-4-piperidyl)-4-[[(1R)-1-[3-amino-2-fluoro-5-(trifluoromethyl)phenyl]ethyl]amino]-8-methyl-pyrido[2,3-d]pyrimidin-7-one;
6-(1-acetyl-4-piperidyl)-4-[[(1R)-1-[5-amino-2-fluoro-3-(trifluoromethyl)phenyl]ethyl]amino]-8-methyl-pyrido[2,3-d]pyrimidin-7-one;
(R)-4-((1-(3-(difluoromethyl)-2-fluorophenyl)ethyl)amino)-8-methyl-6-(pyridazin-4-yl)pyrido[2,3-d]pyrimidin-7(8H)-one;
4-(((R)-1-(3-(difluoromethyl)-2-fluorophenyl)ethyl)amino)-8-methyl-6-(1-oxido-3,6-dihydro-2H-thiopyran-4-yl)pyrido[2,3-d]pyrimidin-7(8H)-one;
4-(((R)-1-(3-(difluoromethyl)-2-fluorophenyl)ethyl)amino)-6-(1-imino-1-oxido-1,2,3,6-tetrahydro-1λ⁶-thiopyran-4-yl)-8-methylpyrido[2,3-d]pyrimidin-7(8H)-one;
4-(((R)-1-(3-(difluoromethyl)-2-fluorophenyl)ethyl)amino)-8-methyl-6-(1-(methylimino)-1-oxido-1,2,3,6-tetrahydro-1λ⁶-thiopyran-4-yl)pyrido[2,3-d]pyrimidin-7(8H)-one;
(R)-4-((1-(3-(difluoromethyl)-2-fluorophenyl)ethyl)amino)-8-methyl-6-(1-oxidotetrahydro-2H-thiopyran-4-yl)pyrido[2,3-d]pyrimidin-7(8H)-one;
4-(4-{[(1R)-1-[3-(difluoromethyl)-2-fluorophenyl]-ethyl]amino}-8-methyl-7-oxo-7H,8H-pyrido[2,3-d]pyrimidin-6-yl)-4-hydroxy-1λ⁶-thiane-1,1-dione;
4-(4-{[(1R)-1-[3-(difluoromethyl)-2-fluorophenyl]ethyl]amino}-8-methyl-7-oxo-7H,8H-pyrido[2,3-d]-pyrimidin-6-yl)-4-fluoro-1λ⁶-thiane-1,1-dione;
4-{[(1R)-1-[3-(difluoromethyl)-2-fluorophenyl]ethyl]amino}-8-methyl-6-(phenylsulfanyl)-7H,8H-pyrido[2,3-d]pyrimidin-7-one;
6-(4-aminooxan-4-yl)-4-{[(1R)-1-[3-(difluoromethyl)-2-fluorophenyl]ethyl]amino}-8-methyl-7H,8H-pyrido-[2,3-d]pyrimidin-7-one;
4-(4-{[(1R)-1-[3-(difluoro-methyl)-2-fluorophenyl]ethyl]amino}-8-methyl-7-oxo-7H,8H-pyrido[2,3-d]pyrimidin-6-yl)-4-methoxy-1λ⁶-thiane-1,1-dione;
6-(4-{[(1R)-1-[3-(difluoromethyl)-2-fluorophenyl]ethyl]amino}-8-methyl-7-oxo-7H,8H-pyrido[2,3-d]pyrimi-din-6-yl)-2λ⁶-thiaspiro[3.3]heptane-2,2-dione;
4-(((R)-1-(3-(difluoromethyl)-2-fluorophenyl)ethyl)amino)-6-(3-hydroxypiperidin-4-yl)-8-methylpyrido[2,3-d]pyrimidin-7(8H)-one;
(R)-4-((1-(3-(difluoromethyl)-2-fluorophenyl)ethyl)amino)-6-(1-imino-1-oxidohexahydro-1λ⁶-thiopyran-4-yl)-8-methylpyrido[2,3-d]pyrimidin-7(8H)-one;
1-acetyl-4-(4-{[(1R)-1-[3-(difluoromethyl)-2-fluorophenyl]ethyl]amino}-8-methyl-7-oxo-7H,8H-pyrido[2,3-d]pyrimidin-6-yl)piperidine-4-carbonitrile;
4-(4-{[(1R)-1-[3-(difluoromethyl)-2-fluorophenyl]ethyl]amino}-8-methyl-7-oxo-7H,8H-pyrido[2,3-d]pyrimidin-6-yl)-4-methyl-1λ⁶-thiane-1,1-dione;
6-(1-acetylpiperidin-4-yl)-4-{[(1R)-1-[2,3-bis(difluoromethyl)phenyl]ethyl]amino}-8-methyl-7H,8H-pyrido[2,3-d]pyrimidin-7-one;
6-(1-acetylpiperidin-4-yl)-4-{[(1R)-1-[3-(difluoromethyl)-2-(fluoromethyl)phenyl]ethyl]amino}-8-methyl-7H,8H-pyrido[2,3-d]pyrimidin-7-one;
2-{3-[(1R)-1-{[6-(1,1-dioxo-3,6-dihydro-2H-1λ⁶-thiopyran-4-yl)-8-methyl-7-oxo-7H,8H-pyrido[2,3-d]pyrimidin-4-yl]amino}ethyl]phenyl}-2,2-difluoroacetonitrile;
2-{3-[(1R)-1-{[6-(1,1-dioxo-3,6-dihydro-2H-1λ⁶-thiopyran-4-yl)-8-methyl-7-oxo-7H,8H-pyrido[2,3-d]pyrimidin-4-yl]amino}ethyl]-2-fluorophenyl}-2,2-difluoroacetonitrile;
4-(4-{[(1R)-1-[3-(2-amino-1,1-difluoroethyl)-2-fluorophenyl]ethyl]amino}-8-methyl-7-oxo-7H,8H-pyrido[2,3-d]pyrimidin-6-yl)-3,6-dihydro-2H-1λ⁶-thiopyran-1,1-dione;
6-(1-acetyl-4-piperidyl)-4-[[(1R)-1-[3-(difluoromethyl)-5-(3-fluoroazetidin-3-yl)phenyl]ethyl]amino]-8-methyl-pyrido[2,3-d]pyrimidin-7-one;
[4-[4-[[(1R)-1-[3-(difluoromethyl)-2-fluoro-phenyl]ethyl]amino]-8-methyl-7-oxo-pyrido[2,3-d]pyrimidin-6-yl]-1-oxo-3,6-dihydro-2H-thiopyran-1-ylidene]cyanamide;
[4-[4-[[(1R)-1-[3-(difluoromethyl)-2-fluoro-phenyl]ethyl]amino]-8-methyl-7-oxo-pyrido[2,3-d]pyrimidin-6-yl]-1-oxo-thian-1-ylidene]cyanamide;
4-(((R)-1-(3-(difluoromethyl)-2-fluorophenyl)ethyl)amino)-6-((1S,4s)-1-imino-4-methoxy-1-oxidohexahydro-1λ⁶-thiopyran-4-yl)-8-methylpyrido[2,3-d]pyrimidin-7(8H)-one;
4-(((R)-1-(3-(difluoromethyl)-2-fluorophenyl)ethyl)amino)-6-((1R,4r)-1-imino-4-methoxy-1-oxidohexahydro-1λ⁶-thiopyran-4-yl)-8-methylpyrido[2,3-d]pyrimidin-7(8H)-one;
4-(((R)-1-(3-(difluoromethyl)-2-fluorophenyl)ethyl)amino)-6-((1S,4s)-4-fluoro-1-(methylimino)-1-oxidohexahydro-1λ⁶-thiopyran-4-yl)-8-methylpyrido[2,3-d]pyrimidin-7(8H)-one;
4-(((R)-1-(3-(difluoromethyl)-2-fluorophenyl)ethyl)amino)-6-((1R,4r)-4-fluoro-1-(methylimino)-1-oxidohexahydro-1λ⁶-thiopyran-4-yl)-8-methylpyrido[2,3-d]pyrimidin-7(8H)-one;
6-{3-acetyl-3-azabicyclo[3.1.0]hexan-1-yl}-4-{[(1R)-1-[3-(difluoromethyl)-2-fluorophenyl]ethyl]amino}-8-methyl-7H,8H-pyrido[2,3-d]pyrimidin-7-one;
6-(1-acetyl-4-piperidyl)-4-[[(1R)-1-[3-[(4-cyclopropylmorpholin-2-yl)-difluoro-methyl]-2-fluoro-phenyl]ethyl]amino]-8-methyl-pyrido[2,3-d]pyrimidin-7-one;
6-((1R,4r)-1-(cyclopropylimino)-4-fluoro-1-oxidohexahydro-1λ⁶-thiopyran-4-yl)-4-(((R)-1-(3-(difluoromethyl)-2-fluorophenyl)ethyl)amino)-8-methylpyrido[2,3-d]pyrimidin-7(8H)-one;
6-((1S,4s)-1-(cyclopropylimino)-4-fluoro-1-oxidohexahydro-1λ⁶-thiopyran-4-yl)-4-(((R)-1-(3-(difluoromethyl)-2-fluorophenyl)ethyl)amino)-8-methylpyrido[2,3-d]pyrimidin-7(8H)-one;
(1R,4r)-1-(cyclopropylimino)-4-(4-(((R)-1-(3-(difluoromethyl)-2-fluorophenyl)ethyl)amino)-8-methyl-7-oxo-7,8-dihydropyrido[2,3-d]pyrimidin-6-yl)hexahydro-1λ⁶-thiopyran-4-carbonitrile 1-oxide;
(1S,4s)-1-(cyclopropylimino)-4-(4-(((R)-1-(3-(difluoromethyl)-2-fluorophenyl)ethyl)amino)-8-methyl-7-oxo-7,8-dihydropyrido[2,3-d]pyrimidin-6-yl)hexahydro-1λ⁶-thiopyran-4-carbonitrile 1-oxide;
4-[[(1R)-1-[3-(difluoromethyl)-2-fluoro-phenyl]ethyl]amino]-8-methyl-6-[1-(oxetan-3-ylimino)-1-oxo-thian-4-yl]pyrido[2,3-d]pyrimidin-7-one;
4-[[(1R)-1-[3-(difluoromethyl)-2-fluoro-phenyl]ethyl]amino]-6-[1-[(4-methoxyphenyl)methoxy]cyclopropyl]-8-methyl-pyrido[2,3-d]pyrimidin-7-one;
2-[2-(difluoromethyl)-6-[(1R)-1-[[6-(1,1-dioxo-3,6-dihydro-2H-thiopyran-4-yl)-8-methyl-7-oxo-pyrido[2,3-d]pyrimidin-4-yl]amino]ethyl]phenyl]acetonitrile;
4-(((R)-1-(3-(difluoromethyl)-2-fluorophenyl)ethyl)amino)-8-methyl-6-((1R,4r)-1-(methylimino)-1-oxidohexahydro-1λ⁶-thiopyran-4-yl)pyrido[2,3-d]pyrimidin-7(8H)-one;
4-(((R)-1-(3-(difluoromethyl)-2-fluorophenyl)ethyl)amino)-8-methyl-6-((S)-1-(oxetan-3-ylimino)-1-oxido-1,2,3,6-tetrahydro-1W⁶-thiopyran-4-yl)pyrido[2,3-d]pyrimidin-7(8H)-one;
4-(((R)-1-(3-(difluoromethyl)-2-fluorophenyl)ethyl)amino)-8-methyl-6-((R)-1-(oxetan-3-ylimino)-1-oxido-1,2,3,6-tetrahydro-1W⁶-thiopyran-4-yl)pyrido[2,3-d]pyrimidin-7(8H)-one;
4-[[(1R)-1-[3-(difluoromethyl)-2-fluoro-phenyl]ethyl]amino]-6-(1-imino-1-oxo-thian-4-yl)-8-methyl-pyrido[2,3-d]pyrimidin-7-one;
6-((1R,4r)-1-(cyclopropylimino)-4-methoxy-1-oxidohexahydro-1λ⁶-thiopyran-4-yl)-4-(((R)-1-(3-(difluoromethyl)-2-fluorophenyl)ethyl)amino)-8-methylpyrido[2,3-d]pyrimidin-7(8H)-one;
6-((1S,4s)-1-(cyclopropylimino)-4-methoxy-1-oxidohexahydro-1λ⁶-thiopyran-4-yl)-4-(((R)-1-(3-(difluoromethyl)-2-fluorophenyl)ethyl)amino)-8-methylpyrido[2,3-d]pyrimidin-7(8H)-one; and
N-[3-(4-{[(1R)-1-[3-(difluoromethyl)-2-fluorophenyl]ethyl]amino}-8-methyl-7-oxo-7H,8H-pyrido[2,3-d]pyrimidin-6-yl)bicyclo[1.1.1]pentan-1-yl]acetamide;
   or a pharmaceutically acceptable salt or a stereoisomer thereof.

In some embodiments, the SOS1 inhibitor is selected from among the compounds in the following table, or a pharmaceutically acceptable salt or a stereoisomer thereof:

Structure

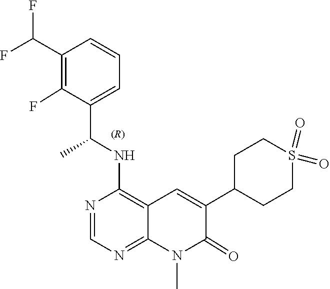

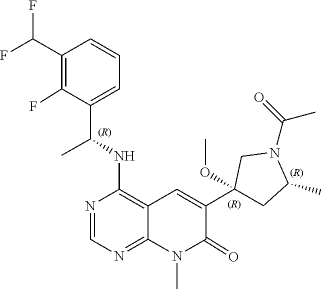

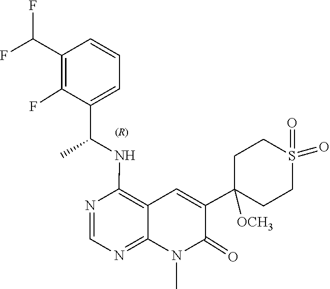

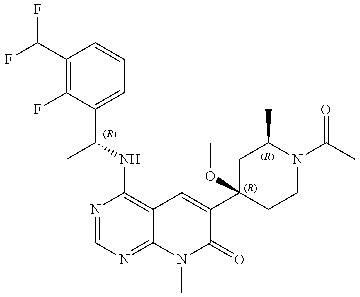

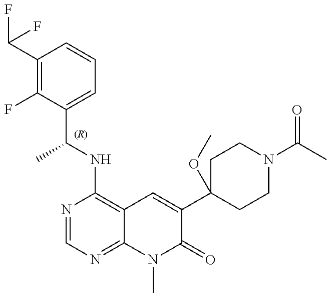

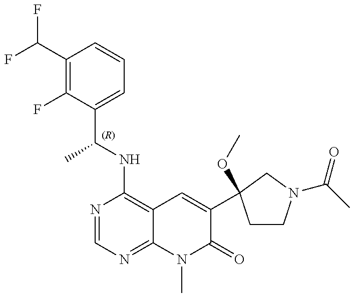

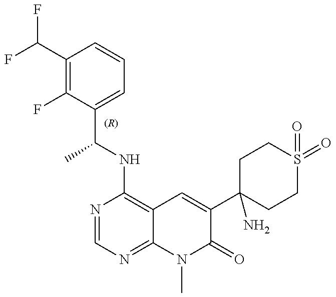

In some embodiments, the SOS1 inhibitor is a compound having the structure of Formula (53-I), (53-II), or (53-III):

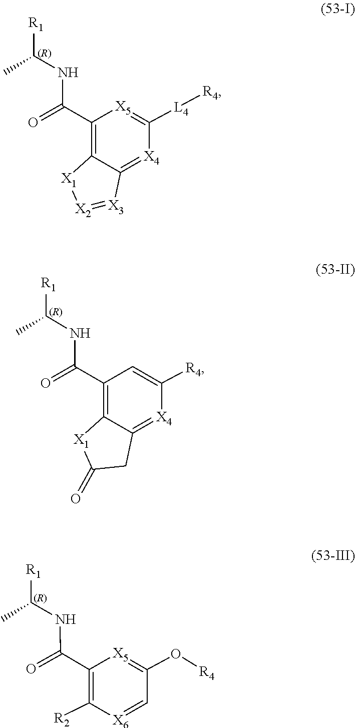

or a pharmaceutically acceptable salt, solvate, isomer, prodrug, or tautomer thereof, wherein:
X₁ is NH or S;
X₂ is CH or N;
X₃ is CH or N;
X₄ is CR₃ or N;
X₅ is CH or N;
X₆ is CH or N;
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, —NH—C₁₆ alkyl, and —NH₂;
R₃ is selected from the group consisting of H, —O—C₁₋₆ alkyl, and —O—C₁₋₆ heteroalkyl;
L₄ is a bond or O; and
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with one or more C₁₋₆ alkyl, —R₄ₐ, —OR₄ₐ, —O—C₁₋₆ alkyl-R₄ₐ, ═O, halogen, —C(O)R₄ₐ, —C(O)OR₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ; wherein R₄a is H, C₁₋₆ alkyl, C₁₋₆ haloalkyl, —C(O)R_{4b}, —C(O)NR_{4b}R_{4c}, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3;
wherein each R_{4b} is independently H, C₁₋₆ alkyl; and
   wherein each R_{4c} is independently H or C₁₋₆ alkyl.

In some embodiments, the present invention is directed to a method of treating a subject having a disease or disorder characterized by SHP2-mediated activation of a RAS protein comprising administering to a subject in need of such treatment a therapeutically effective amount of a SOS1 inhibitor having the structure of Formula (53-I), (53-II), (53-III), or a pharmaceutically acceptable salt, solvate, hydrate, tautomer, or isomer thereof.

In some embodiments, the method of inhibiting SOS1 in a subject comprises administering to the subject in need of such treatment a therapeutically effective amount of a SOS1 inhibitor having the structure of Formula (53-I), (53-II), (53-III), or a pharmaceutically acceptable salt, solvate, hydrate, tautomer, or isomer thereof

In some embodiments, the present invention is directed to a method of inhibiting the interaction of SOS1 and a RAS-family protein in a cell or inhibiting the interaction of SOS1 and RAC1 in a cell, comprising administering to the cell a SOS1 inhibitor having the structure of Formula (53-I), (53-II), (53-III), or a pharmaceutically acceptable salt, solvate, hydrate, tautomer, or isomer thereof.

In some embodiments, the present invention is directed to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof a therapeutically effective amount of a SOS1 inhibitor having the structure of Formula (53-I), (53-II), (53-III), or a pharmaceutically acceptable salt, solvate, hydrate, tautomer, or isomer thereof.

In some embodiments, the present invention is directed to a method of treating or preventing cancer in a subject in need thereof, comprising administering to the subject a therapeutically effective amount of a SOS1 inhibitor having the structure of Formula (53-I), (53-II), (53-III), or a pharmaceutically acceptable salt, solvate, hydrate, tautomer, or isomer thereof.

In some embodiments, the SOS1 inhibitor is a compound having the structure of Formula (53-Ia), (53-IIa), or (53-IIIa):

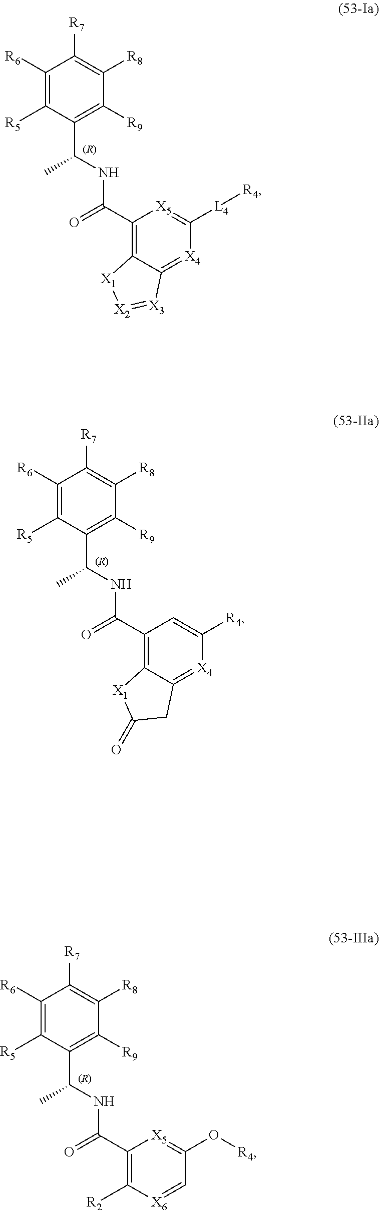

or a pharmaceutically acceptable salt, solvate, isomer, prodrug, or tautomer thereof, wherein X₁, X₂, X₃, X₄, X₅, X₆, R₂, L₄, and R₄ are as defined in Formula (53-I), (53-II), or (53-III);
R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, ═O, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, 3-8 membered cycloalkyl, 3-14 membered heterocyclyl optionally substituted with R₁₀, 6-10 membered aryl, or 5-10 membered heteroaryl, or any two adjacent R⁵, R⁶, R⁷, R⁸, and R⁹ forms an optionally substituted 3-14 membered fused ring;
R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and
R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In some embodiments, the SOS1 inhibitor is a compound having the structure of Formula (53-II-1):

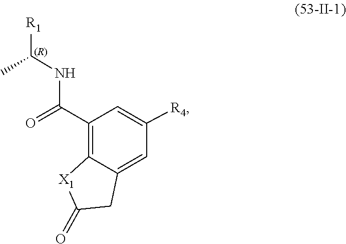

or a pharmaceutically acceptable salt, solvate, isomer, prodrug, or tautomer thereof, wherein R₁ and R₄ are as defined in Formula (II).

In some embodiments, the SOS1 inhibitor is a compound selected from the group consisting of the compounds in the following table, or a pharmaceutically acceptable salt, solvate, isomer, prodrug, or tautomer thereof:

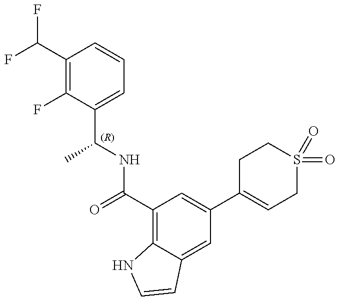

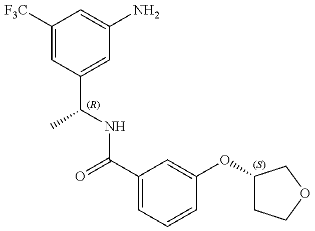

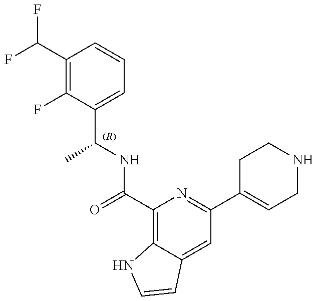

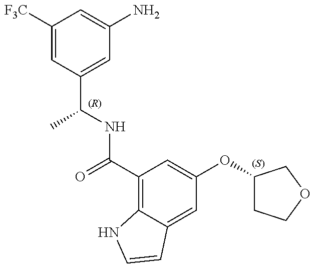

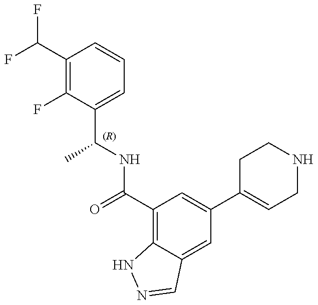

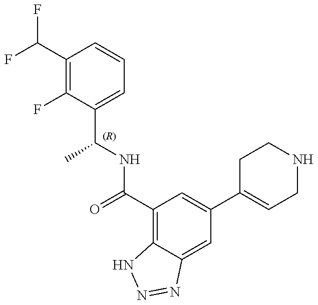

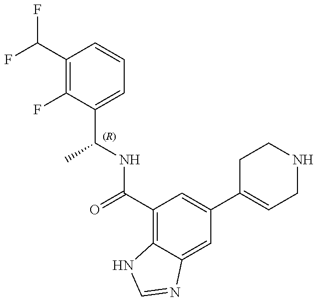

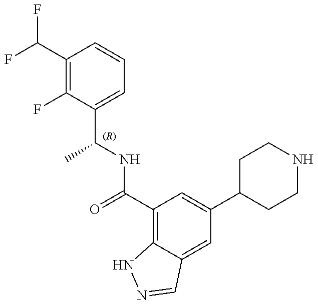

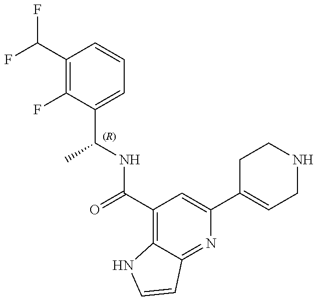

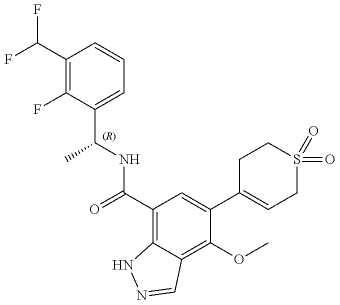

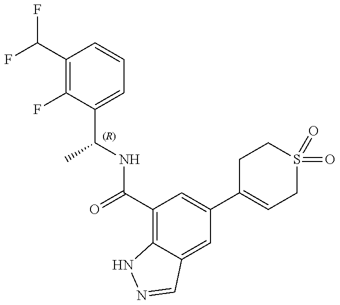

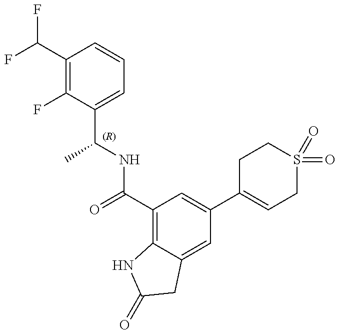

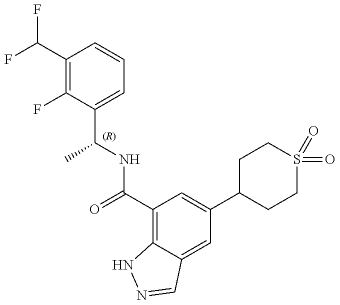

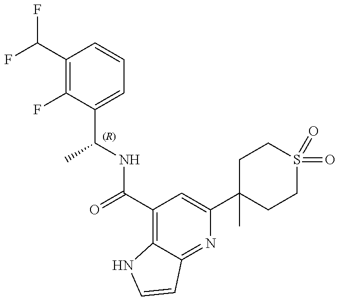

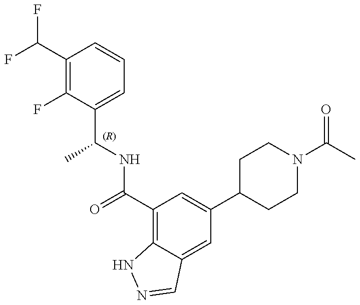

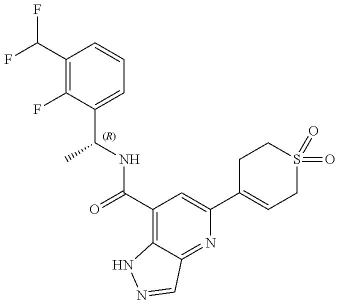

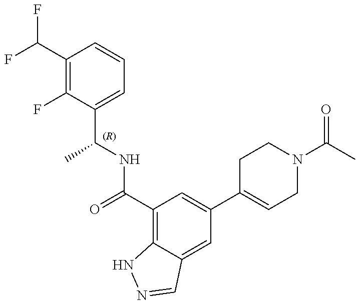

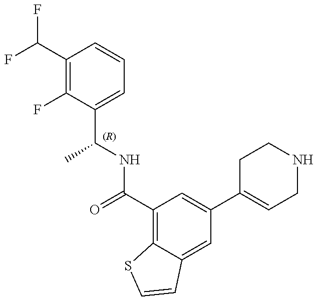

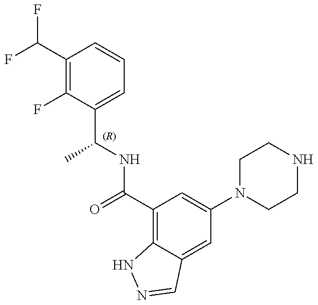

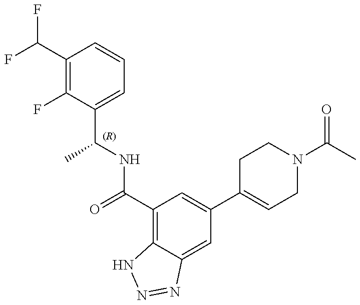

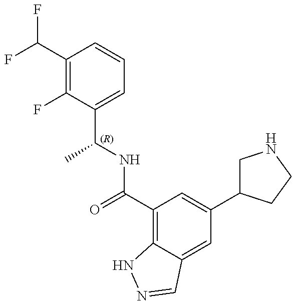

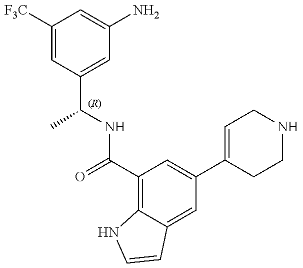

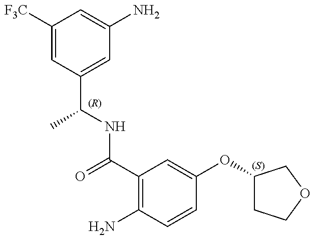

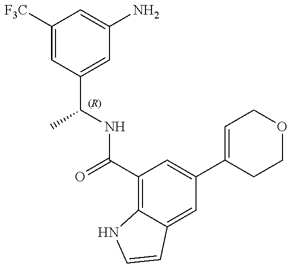

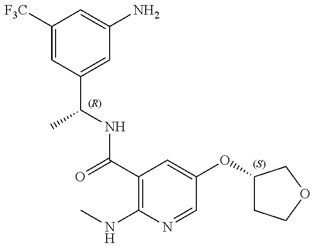

In some embodiments, the SOS1 inhibitor is BI-3406, having the structure:

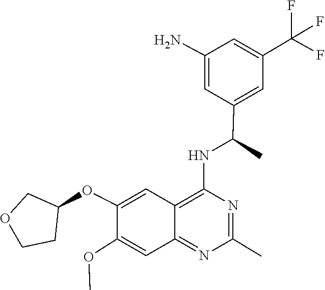 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer, prodrug, or tautomer thereof.

In some embodiments, the SOS1 inhibitor is BI-1701963 or a pharmaceutically acceptable salt, solvate, isomer, prodrug, or tautomer thereof.

In some embodiments, the SOS1 inhibitor is BAY-293, having the structure:

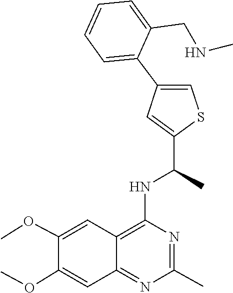

or a pharmaceutically acceptable salt, solvate, isomer, prodrug, or tautomer thereof.

In some embodiments, the SOS1 inhibitor is SDR5 or a pharmaceutically acceptable salt, solvate, isomer, prodrug, or tautomer thereof.

In some embodiments, the SOS1 inhibitor is Compound SOS1-(A) (also called RMC-0331), having the structure:

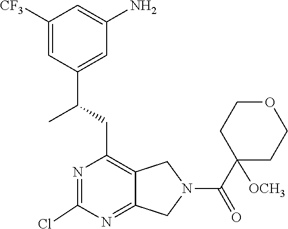 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, prodrug, or tautomer thereof. SOS1-(A) is a compound of Formula (42-I).

In some embodiments, the SOS1 inhibitor is Compound SOS1-(B), or a pharmaceutically acceptable salt, solvate, isomer, prodrug, or tautomer thereof. Compound SOS1-(B) falls within the scope of Formula (48-I).

In some embodiments, the SOS1 inhibitor is Compound SOS1-(C), or a pharmaceutically acceptable salt, solvate, isomer, prodrug, or tautomer thereof. Compound SOS1-(C) falls within the scope of Formula (48-I).

The SOS1 inhibitor dose may range from a dose sufficient to elicit a response to the maximum tolerated dose. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses, such as from 100 mg to 1300 mg, from 200 mg to 1300 mg, from 600 mg to 1300 mg, from 700 mg to 1200 mg, or from 800 mg to 1000 mg. In one embodiment, the compositions are in the form of a tablet that can be scored. The SOS1 inhibitor can be dosed once per day, twice per day, three times per day, or four times per day. In some aspects, SOS1 inhibitor is dosed once per day. In some aspects, SOS1 inhibitor is dosed twice per day. Dosing may be done with or without food. The dosing schedule may suitably be every day of a 28-day schedule, or 21 or more days of a 28-day schedule.

### RAS and RAS Mutations

In some embodiments, the method of the present invention comprises administering to the subject a therapeutically effective amount of a RAS inhibitor selected from the group consisting of a RAS(ON) inhibitor and a RAS(OFF) inhibitor, and a combination thereof. In some embodiments, the Ras protein is wild-type, and the RAS inhibitor targets a wild-type RAS protein. In some embodiments, the RAS inhibitor targets KRAS, NRAS, or HRAS. In some embodiments, the RAS inhibitor targets two or more of KRAS, NRAS, or HRAS.

In some embodiments, the RAS inhibitor targets a RAS protein having a mutation. In some embodiments, the RAS inhibitor is a RAS mutant specific inhibitor. In some embodiments, the RAS inhibitor targets a KRAS mutant, a NRAS mutant, or an HRAS mutant. In certain embodiments, RAS mutant is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, Y96D, or G13V, and combinations thereof,
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof, and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof,
or a combination of any of the foregoing (e.g., both K-Ras G12C and K-Ras G13C). In some embodiments, the cancer comprises a Ras mutation selected from the group consisting of G12C, G13C, G12A, G12D, G13D, G12S, G13S, G12V and G13V. In some embodiments, the cancer comprises at least two Ras mutations selected from the group consisting of G12C, G13C, G12A, G12D, G13D, G12S, G13S, G12V and G13V. In some embodiments, the cancer comprises at least a G12C mutation and a Y96D mutation. Mutations at these positions may result in RAS-driven tumors.

In some embodiments, the RAS inhibitor targets a wild-type RAS protein. In some embodiments, the Ras inhibitor targets RAS^{amp}. In some embodiments, the RAS protein is KRAS. In some embodiments, the RAS protein is NRAS. In some embodiments, a RAS inhibitor targets both a KRAS protein and an NRAS protein. In some embodiments, the RAS inhibitor targets a RAS protein mutation. In some embodiments, the RAS protein mutation is at a position selected from the group consisting of G12, G13, Q61, A146, K117, L19, Q22, V14, A59, and a combination thereof. In some embodiments, the mutation is selected from the group consisting of G12, G13, and Q61. In some embodiments, the mutation is selected from the group consisting of G12C, G12D, G12A, G12S, G12V, G13C, G13D, Q61K, and Q61L.

### RAS Inhibitors

According to some embodiments of the present disclosure, the method comprises treating a subject having a RAS protein-related disease or disorder, the method comprising administering to a subject in need of such treatment (a) a therapeutically effective amount of a SOS1 inhibitor or a pharmaceutically acceptable salt, solvate, isomer, prodrug, or tautomer thereof; and (b) a therapeutically effective amount of a RAS inhibitor selected from the group consisting of a RAS(ON) inhibitor and a RAS(OFF) inhibitor, and a combination thereof.

In some embodiments, the RAS inhibitor is a RAS(OFF) inhibitor known in the art disclosed herein. The RAS(OFF) inhibitor may be any one or more of the RAS(OFF) inhibitors disclosed in any one of WO 2022066805, WO 2022066646, WO 2022063297, WO 2022061251, WO 2022056307, WO 2022052895, WO 2022047093, WO 2022042630, WO 2022040469, WO 2022037560, WO 2022031678, WO 2022028492, WO 2022028346, WO 2022026726, WO 2022026723, WO 2022015375, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223, each of which is incorporated herein by reference in its entirety. In some embodiments, the RAS(OFF) inhibitor is selected from sotorasib (AMG 510), adagrasib (MRTX849), MRTX1257, JNJ-74699157 (ARS-3248), LY3537982, ARS-853, ARS-1620, GDC-6036, BPI-421286, JDQ443, and JAB-21000. In some embodiments, the RAS(OFF) inhibitor selectively targets RAS G12C.

In some embodiments, the compositions and methods described herein utilize a RAS inhibitor that is a RAS(ON) inhibitor known in the art or disclosed herein. In some embodiments, the RAS inhibitor is a RAS(ON) inhibitor. In some embodiments, the RAS(ON) inhibitor is an inhibitor selective for RAS G12C, RAS G13D, or RAS G12D. In some embodiments, the RAS(ON) inhibitor is a RAS(ON)^{MULTI} inhibitor. In some embodiments, the RAS(ON) inhibitor is RMC-6236, RMC-6291, RMC-8839, or RMC-9805.

The RAS(ON) inhibitor may be any one or more of the RAS(ON) inhibitors disclosed in WO 2020/132597, or any one of WO 2021/091956, WO 2021/091982, WO 2021/091967, and WO 2022/060836, each of which is incorporated herein by reference in its entirety, or a compound described by a Formula of any one of WO 2020/132597, or any one of WO 2021/091956, WO 2021/091982, WO 2021/091967, and WO 2022/060836, or a pharmaceutically acceptable salt thereof.

In some embodiments, the RAS inhibitor is a compound disclosed in WO 2021/091956.

In some embodiments, the RAS(ON) inhibitor is a compound, or pharmaceutically acceptable salt thereof, having the structure of Formula A00:

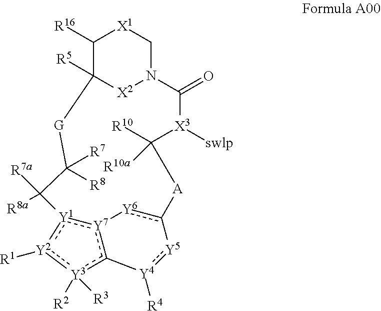

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
swIp (Switch I/P-loop) refers to an organic moiety that non-covalently binds to both the Switch I binding pocket and residues 12 or 13 of the P-loop of a Ras protein (see, e.g., Johnson et al., 292:12981-12993 (2017), incorporated herein by reference);
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R¹⁰ is hydrogen, halo, hydroxy, C₁-C₃ alkoxy, or C₁-C₃ alkyl;
R^{10a} is hydrogen or halo; and
R¹⁶ is hydrogen or C₁-C₃ alkyl (e.g., methyl).

In some embodiments, the resulting compound is capable of achieving an IC50 of 2 uM or less (e.g., 1.5 uM, 1 uM, 500 nM, or 100 nM or less) in the Ras-RAF disruption assay protocol described herein.

In some embodiments, the disclosure features a compound, or pharmaceutically acceptable salt thereof, of structural Formula AL

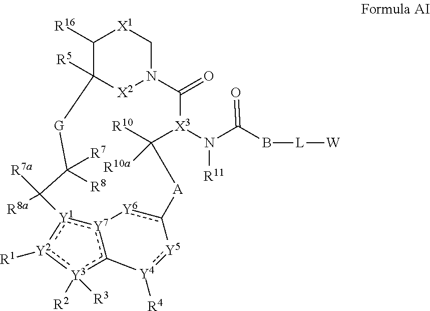

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl (e.g., methyl).

In some embodiments, the disclosure features a compound, or pharmaceutically acceptable salt thereof, of structural Formula Ala:

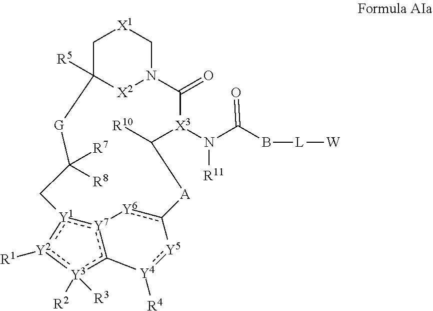

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula AIb:

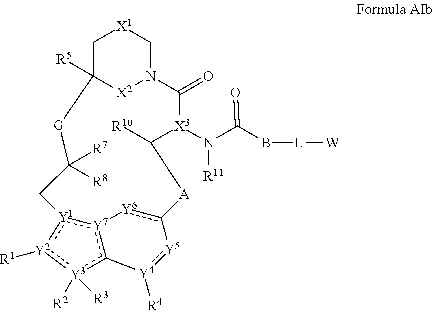

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula AI and subformula thereof, G is optionally substituted C₁-C₄ heteroalkylene.

In some embodiments, the RAS(ON) inhibitor has the structure of Formula AIc, or a pharmaceutically acceptable salt thereof:

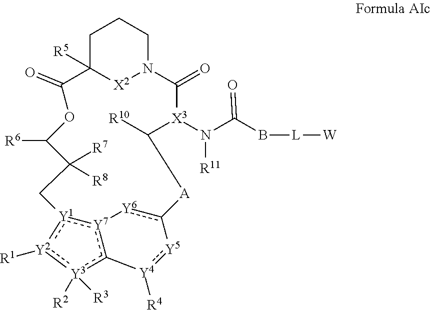

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula AI and subformula thereof, X² is NH. In some embodiments of Formula AI and subformula thereof, X³ is CH.

In some embodiments of Formula AI and subformula thereof, R¹¹ is hydrogen. In some embodiments of Formula AI and subformula thereof, R¹¹ is C₁-C₃ alkyl.

In some embodiments of Formula AI and subformula thereof, R¹¹ is methyl.

In some embodiments, the RAS(ON) inhibitor has the structure of Formula AId, or a pharmaceutically acceptable salt thereof:

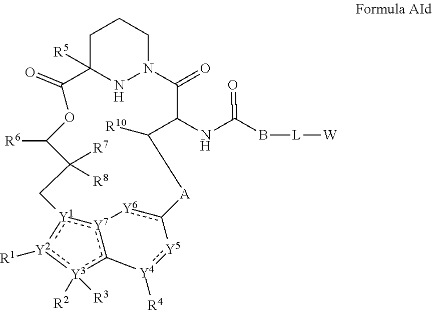

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of compounds of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH(Br)—. In some embodiments, X¹ is —CH(CH₃)—.

In some embodiments of Formula AI and subformula thereof, R³ is absent.

In some embodiments of Formula AI and subformula thereof, R⁴ is hydrogen.

In some embodiments of Formula AI and subformula thereof, R⁵ is hydrogen. In some embodiments of Formula AI and subformula thereof, R⁵ is C₁-C₄ alkyl optionally substituted with halogen. In some embodiments of Formula AI and subformula thereof, R⁵ is methyl.

In some embodiments of Formula AI and subformula thereof, Y⁴ is C.

In some embodiments of Formula AI and subformula thereof, Y⁵ is CH. In some embodiments of Formula AI and subformula thereof, Y⁶ is CH. In some embodiments of Formula AI and subformula thereof, Y¹ is C. In some embodiments of Formula AI and subformula thereof, Y² is C. In some embodiments of Formula AI and subformula thereof, Y³ is N. In some embodiments of Formula AI and subformula thereof, Y⁷ is C.

In some embodiments, the RAS(ON) inhibitor has the structure of Formula AIe, or a pharmaceutically acceptable salt thereof:

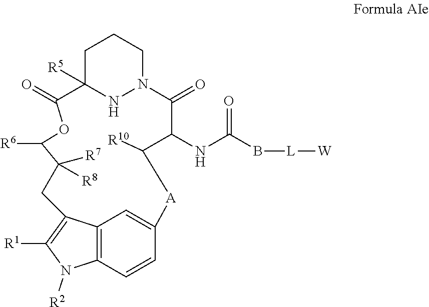

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl; R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R_{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula AI and subformula thereof, R⁶ is hydrogen.

In some embodiments of Formula AI and subformula thereof, R² is hydrogen, cyano, optionally substituted C₁-C₆ alkyl, optionally substituted 3 to 6-membered cycloalkyl, or optionally substituted 3 to 6-membered heterocycloalkyl. In some embodiments of Formula AI and subformula thereof, R² is optionally substituted C₁-C₆ alkyl, such as ethyl. In some embodiments of Formula AI and subformula thereof, R² is fluoro C₁-C₆ alkyl, such as —CH₂CH₂F, —CH₂CHF₂, or —CH₂CF₃.

In some embodiments of Formula AI and subformula thereof, R⁷ is optionally substituted C₁-C₃ alkyl. In some embodiments of Formula AI and subformula thereof, R⁷ is C₁-C₃ alkyl.

In some embodiments of Formula AI and subformula thereof, R⁸ is optionally substituted C₁-C₃ alkyl. In some embodiments of Formula AI and subformula thereof, R⁸ is C₁-C₃ alkyl, such as methyl.

In some embodiments, the RAS(ON) inhibitor has the structure of Formula AIf, or a pharmaceutically acceptable salt thereof:

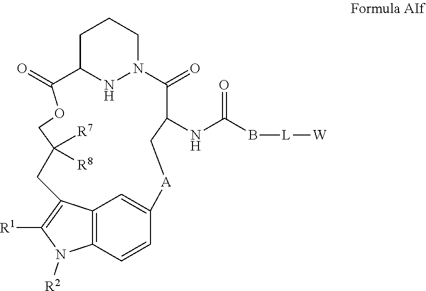

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, R¹ is 5 to 10-membered heteroaryl. In some embodiments, R¹ is optionally substituted 6-membered aryl or optionally substituted 6-membered heteroaryl.

In some embodiments of Formula AI and subformula thereof, R₁ is

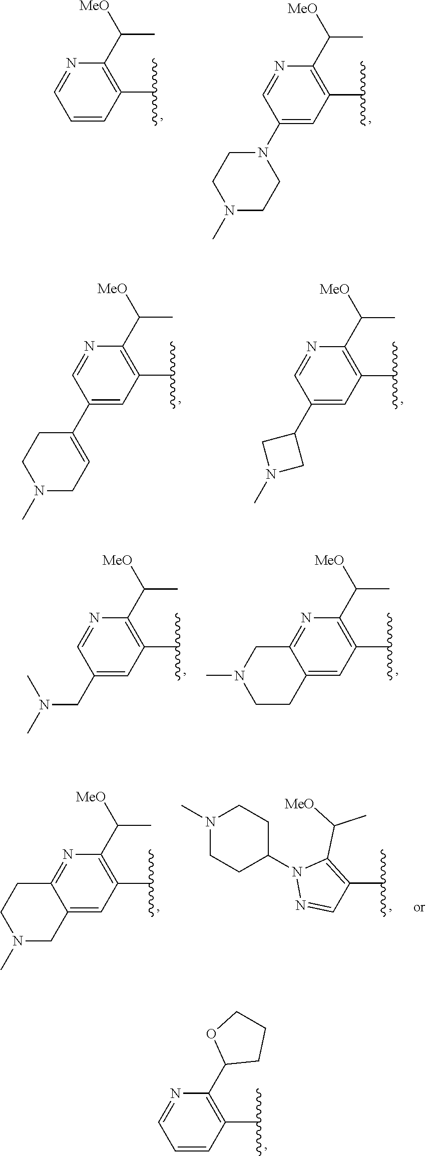

or a stereoisomer thereof.

In some embodiments, R₁ is

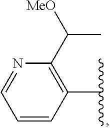 [SMILES: *c1cccnc1C(C)OC]

or a stereoisomer thereof

In some embodiments, R₁ is

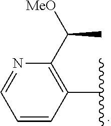 [SMILES: *c1cccnc1[C@H](C)OC]

In some embodiments, R₁ is

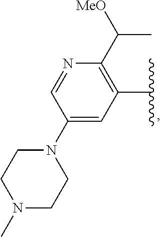 [SMILES: *c1cc(N2CCN(C)CC2)cnc1C(C)OC]

or a stereoisomer thereof.

In some embodiments, R₁ is

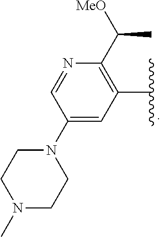 [SMILES: *c1cc(N2CCN(C)CC2)cnc1[C@H](C)OC]

In some embodiments, the RAS(ON) inhibitor has the structure of Formula AIg, or a pharmaceutically acceptable salt thereof:

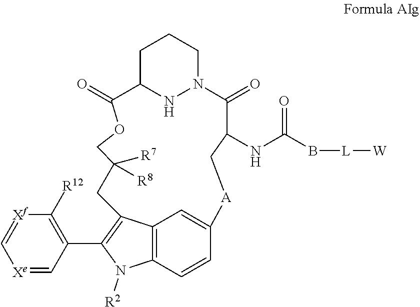

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is N, CH, or CR¹⁷;
X^{f} is N or CH;
R¹² is optionally substituted C₁-C₆ alkyl or optionally substituted C₁-C₆ heteroalkyl; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments of Formula AI and subformula thereof, X^{e} is N and X^{f} is CH. In some embodiments, X^{e} is CH and X^{f} is N. In some embodiments, X^{e} is CR¹⁷ and X^{f} is N.

In some embodiments of Formula AI and subformula thereof, R¹² is optionally substituted C₁-C₆ heteroalkyl.

In some embodiments, R¹² is

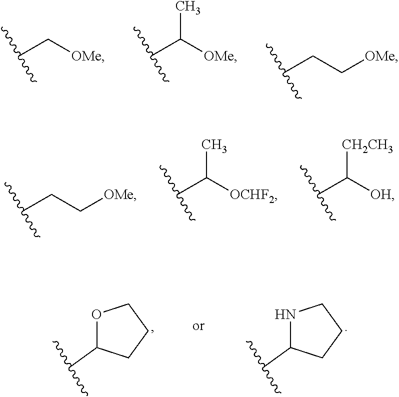

In some embodiments, the RAS(ON) inhibitor has the structure of Formula AIh, or a pharmaceutically acceptable salt thereof:

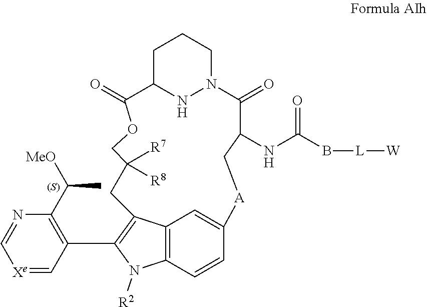

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is CH, or CR¹⁷; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor has the structure of Formula AIi, or a pharmaceutically acceptable salt thereof:

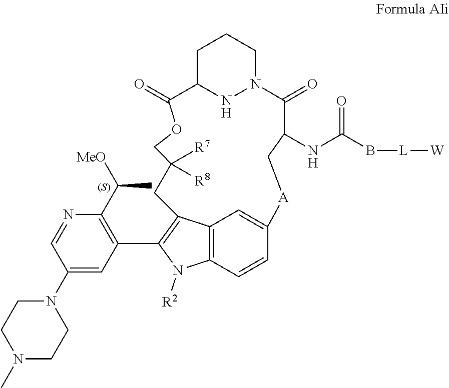

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, A is optionally substituted 6-membered arylene. In some embodiments, A has the structure:

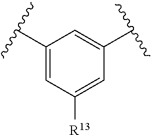

wherein R¹³ is hydrogen, hydroxy, amino, cyano, optionally substituted C₁-C₆ alkyl, or optionally substituted C₁-C₆ heteroalkyl.

In some embodiments, R¹³ is hydrogen.

In some embodiments, R¹³ is hydroxy.

In some embodiments, A is an optionally substituted 5 to 10-membered heteroarylene. In some embodiments, A is:

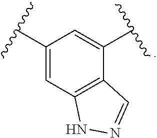 [SMILES: *c1cc(*)c2cn[nH]c2c1]

In some embodiments, A is optionally substituted 5 to 6-membered heteroarylene.

In some embodiments, A is:

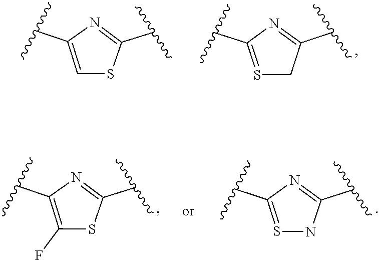

In some embodiments, A is

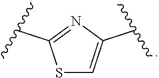 [SMILES: *c1csc(*)n1]

In some embodiments of Formula AI and subformula thereof, B is —CHR⁹—

In some embodiments, R⁹ is optionally substituted C₁-C₆ alkyl or optionally substituted 3 to 6-membered cycloalkyl.

In some embodiments, R⁹ is:

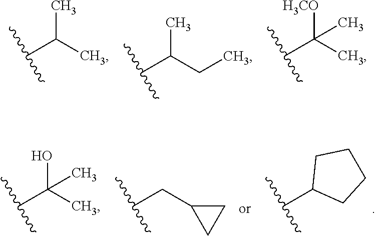

In some embodiments, R⁹ is

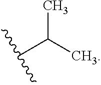 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, B is optionally substituted 6-membered arylene.

In some embodiments, B is 6-membered arylene.

In some embodiments, B is:

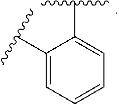 [SMILES: *c1ccccc1*]

In some embodiments B is absent.

In some embodiments of Formula AI and subformula thereof, R⁷ is methyl.

In some embodiments of Formula AI and subformula thereof, R⁸ is methyl.

In some embodiments of Formula AI and subformula thereof, R¹⁶ is hydrogen.

In some embodiments of Formula AI and subformula thereof, the linker is the structure of Formula AII:

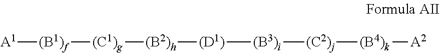

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹—(B¹)_{f}—(C¹)_{g}—(B²)ₕ— to —(B³)ᵢ—(C²)ⱼ—(B⁴)ₖ-A². In some embodiments, the linker is acyclic. In some embodiments, the linker has the structure of Formula AIIa:

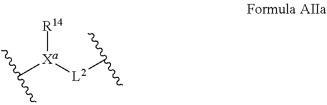

wherein X^{a} is absent or N;
R¹⁴ is absent, hydrogen or optionally substituted C₁-C₆ alkyl or optionally substituted C₁-C₃ cycloalkyl; and
L² is absent, —C(O)—, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

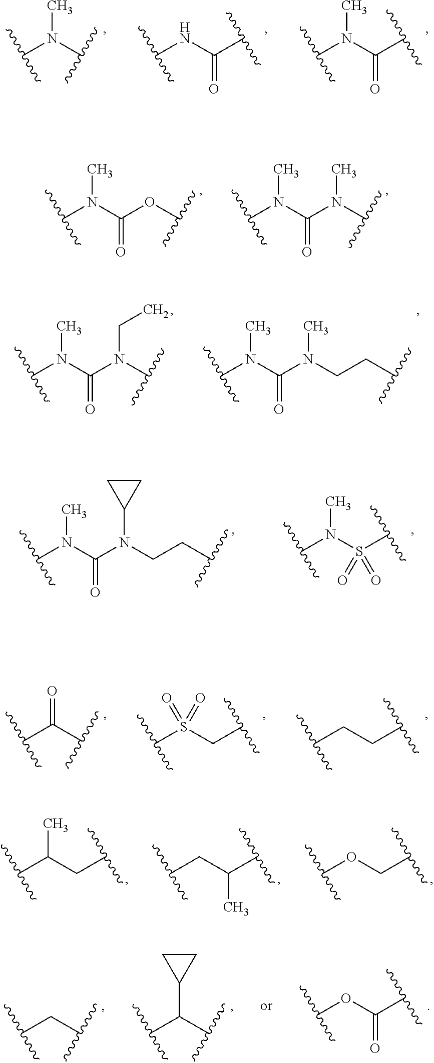

In some embodiments, L is

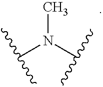 [SMILES: *N(*)C]

In some embodiments, L is

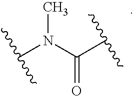 [SMILES: *C(=O)N(*)C]

In some embodiments, linker is or comprises a cyclic group.

In some embodiments of Formula AI and subformula thereof, the linker has the structure of Formula AIIb:

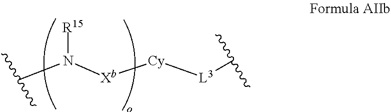

wherein o is 0 or 1;
X^{b} is C(O) or SO₂;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —C(O)—, —SO₂—, optionally substituted C₁-C₄ alkylene or optionally substituted C₁-C₄ heteroalkylene.

In some embodiments, the linker has the structure:

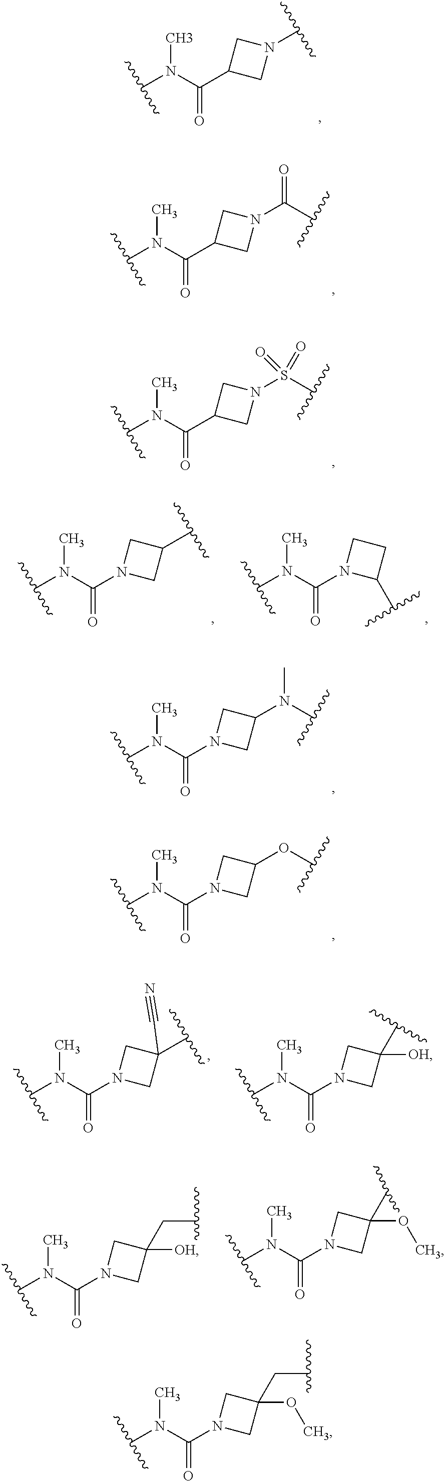

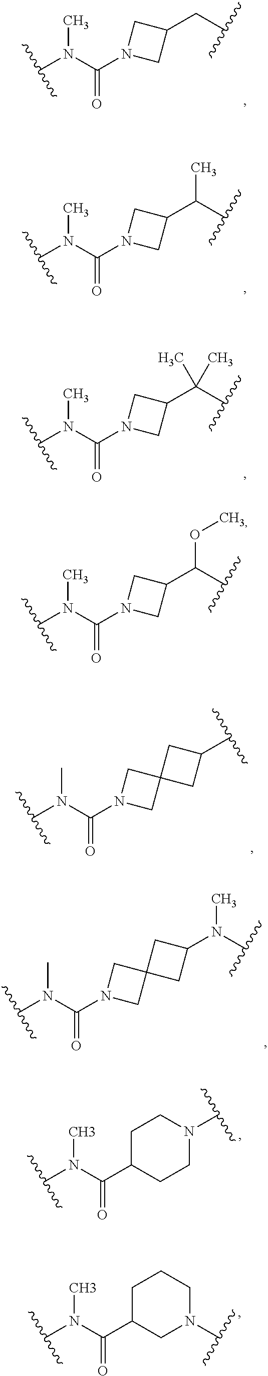

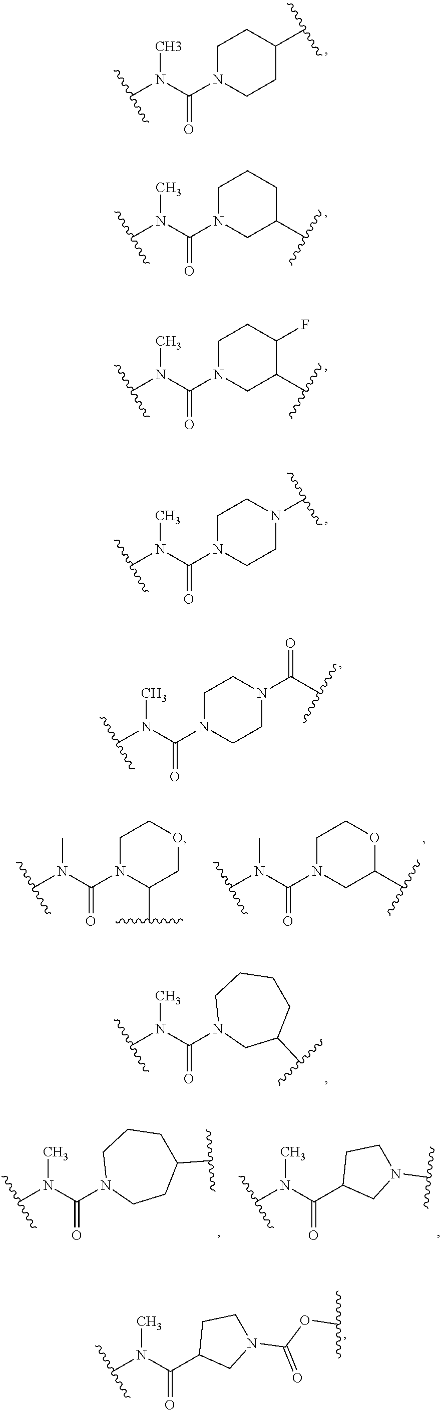

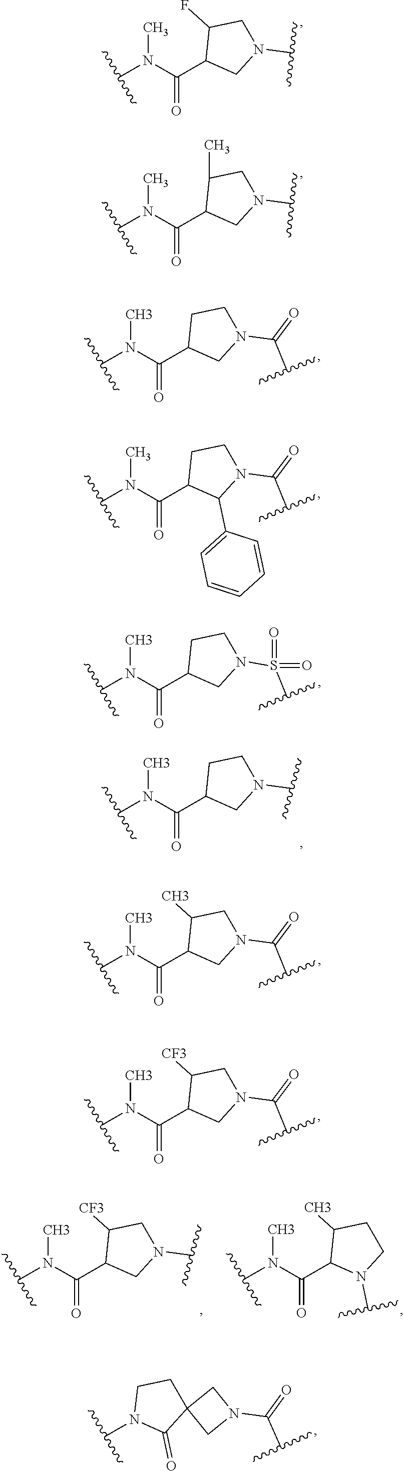

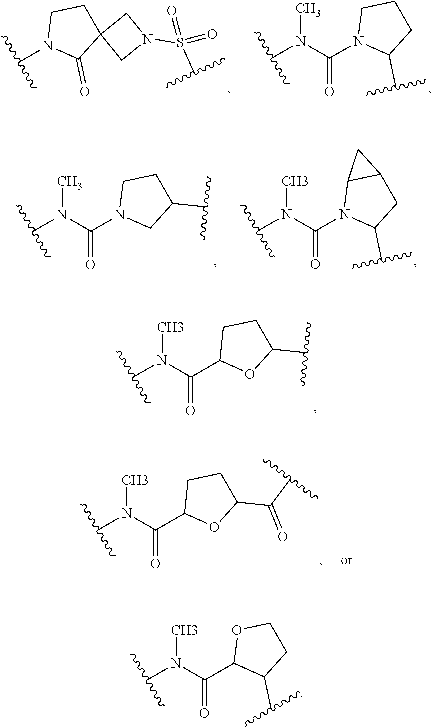

In some embodiments of Formula AI and subformula thereof, W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 8-membered heterocycloalkyl, optionally substituted 3 to 8-membered cycloalkyl, or 3 to 8-membered heteroaryl.

In some embodiments of Formula AI and subformula thereof, W is hydrogen. In some embodiments of Formula AI and subformula thereof, W is optionally substituted amino. In some embodiments of Formula AI and subformula thereof, W is —NHCH₃ or —N(CH₃)₂. In some embodiments of Formula AI and subformula thereof, W is optionally substituted C₁-C₄ alkoxy. In some embodiments, W is methoxy or iso-propoxy. In some embodiments of Formula AI and subformula thereof, W is optionally substituted C₁-C₄ alkyl. In some embodiments, W is methyl, ethyl, iso-propyl, tert-butyl, or benzyl.

In some embodiments of Formula AI and subformula thereof, W is optionally substituted amido.

In some embodiments, W is

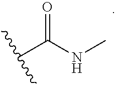 [SMILES: *C(=O)NC]

In some embodiments, W is

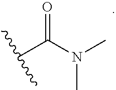 [SMILES: *C(=O)N(C)C]

In some embodiments of Formula AI and subformula thereof, W is optionally substituted C₁-C₄ hydroxyalkyl.

In some embodiments, W is

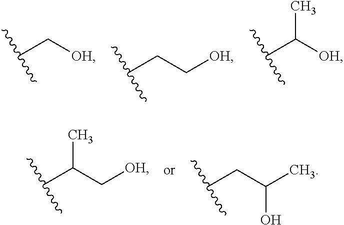

In some embodiments of Formula AI and subformula thereof, W is optionally substituted C₁-C₄ aminoalkyl.

In some embodiments, W is

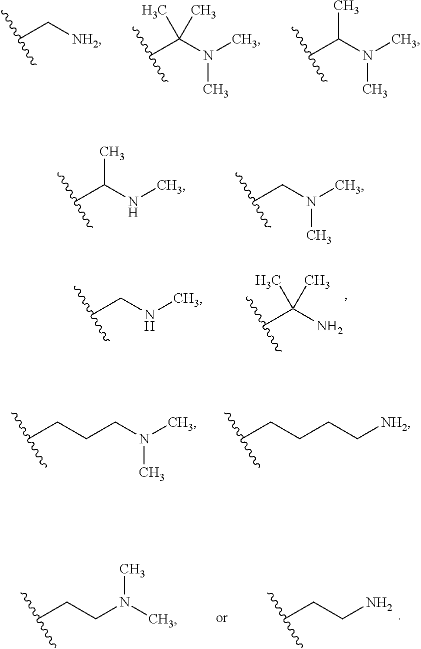

In some embodiments of Formula AI and subformula thereof, W is optionally substituted C₁-C₄ haloalkyl.

In some embodiments, W is

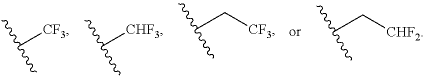

In some embodiments of Formula AI and subformula thereof, W is optionally substituted C₁-C₄ guanidinoalkyl.

In some embodiments, W is

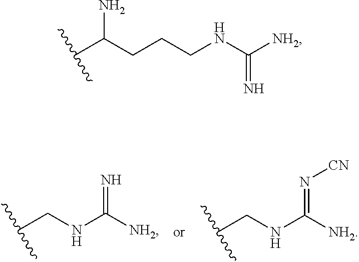 [SMILES: *C(N)CCCNC(=N)N ; *CNC(=N)N ; *CN/C(N)=N/C#N]

In some embodiments of Formula AI and subformula thereof, W is C₀-C₄ alkyl optionally substituted 3 to 11-membered heterocycloalkyl. In some embodiments, W is

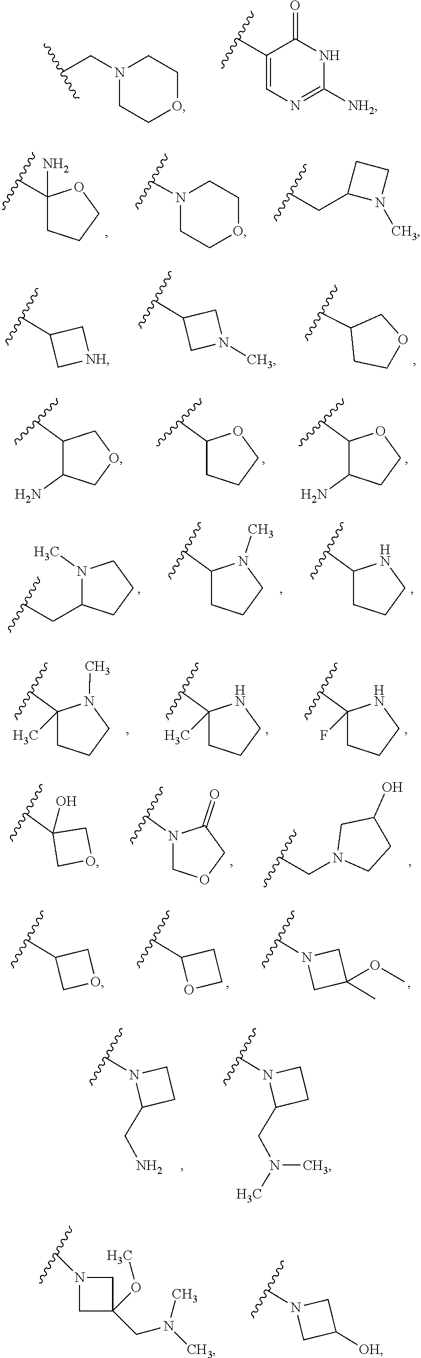

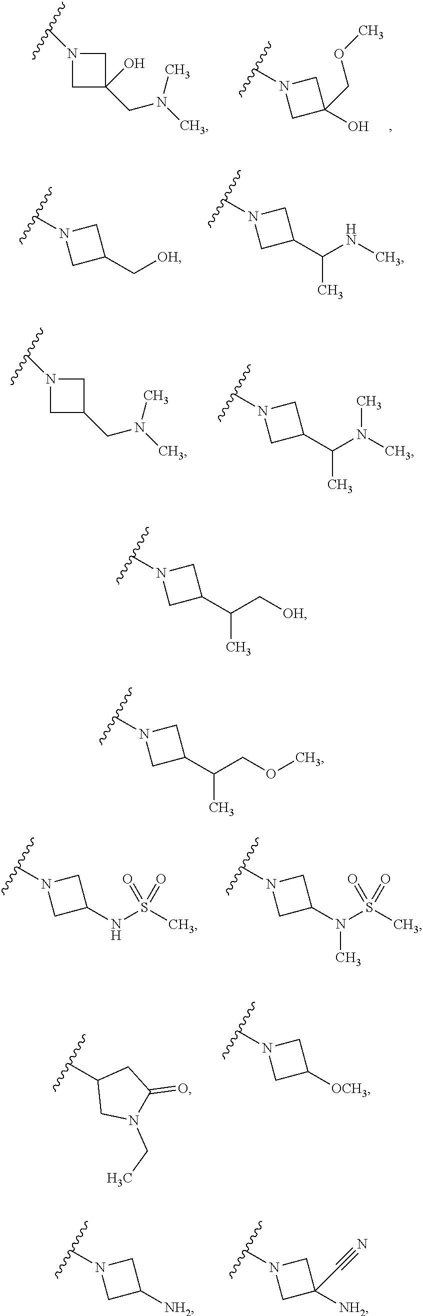

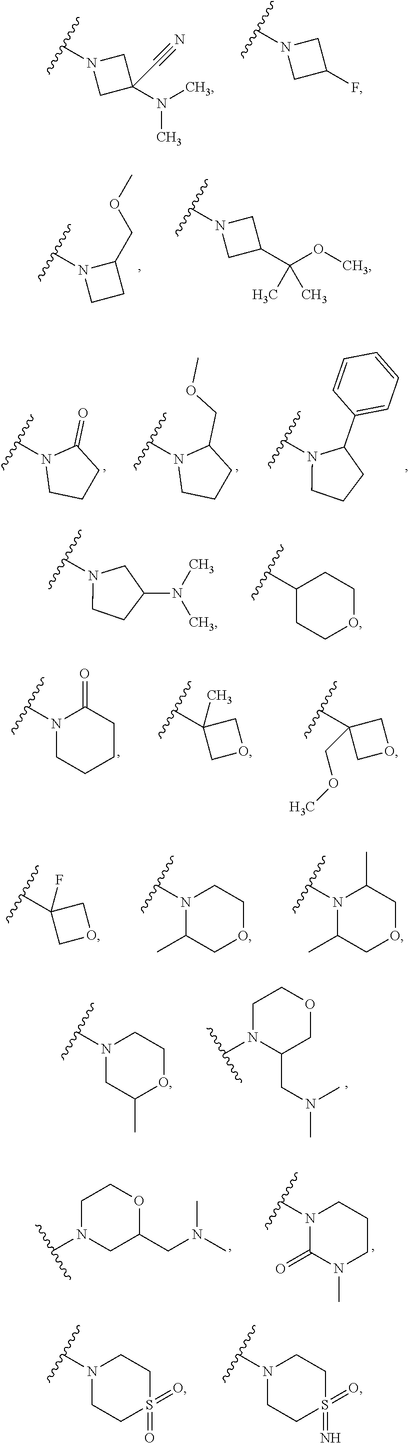

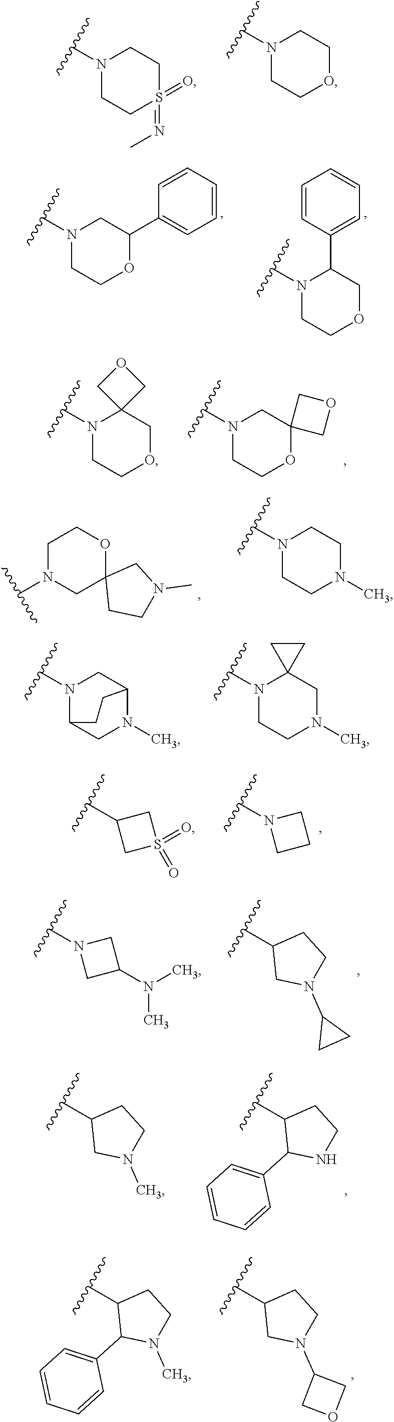

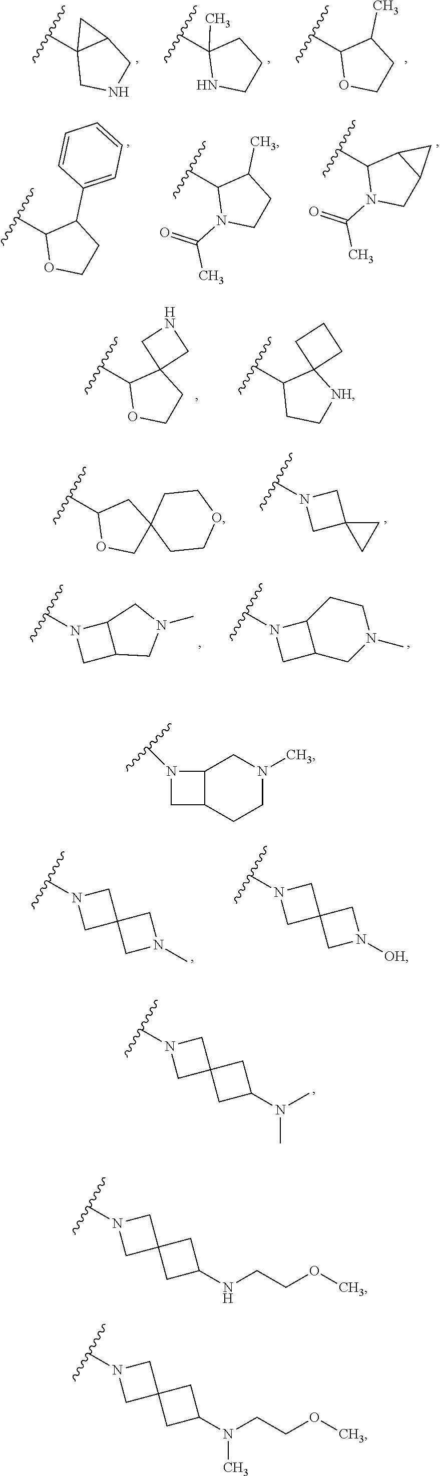

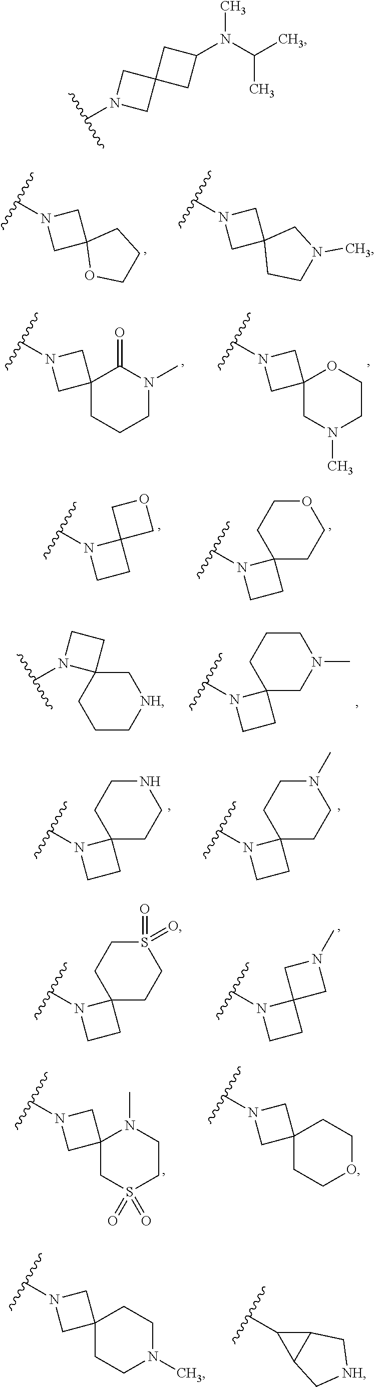

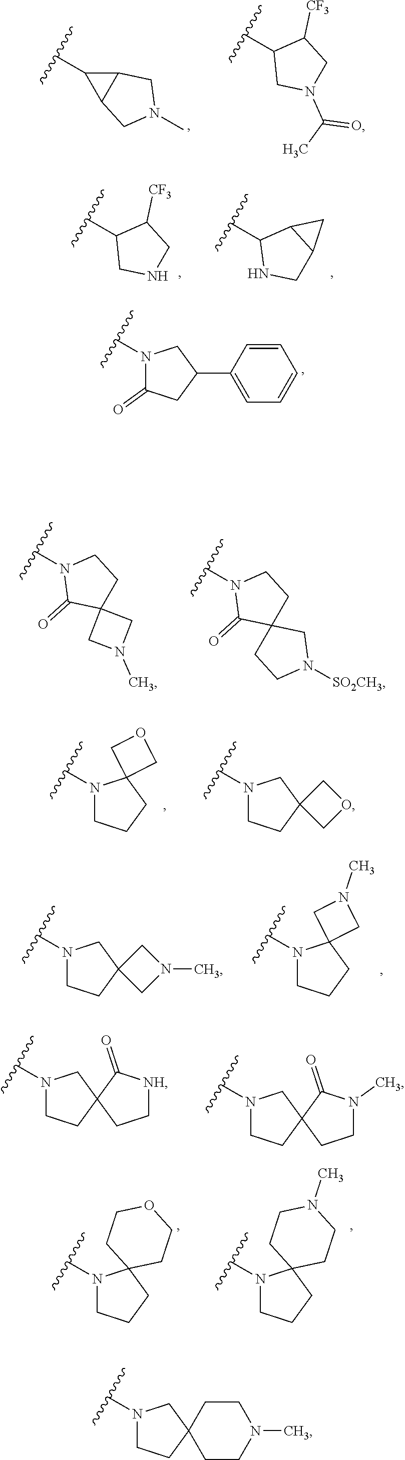

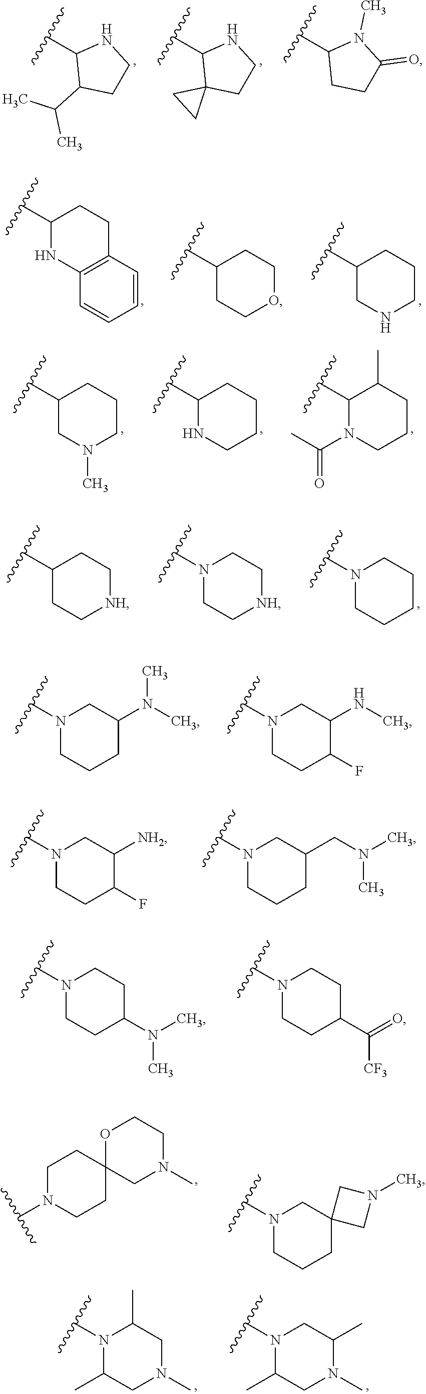

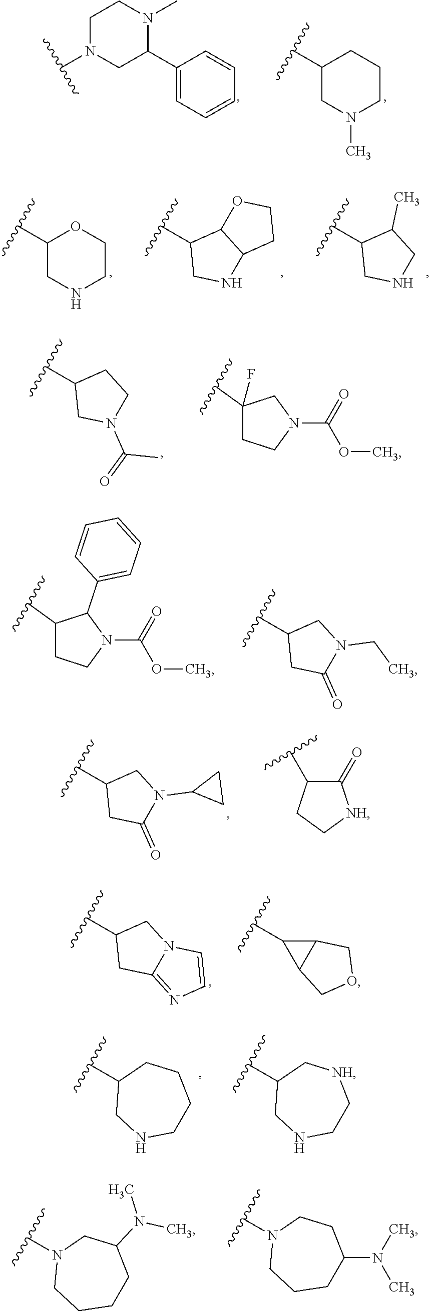

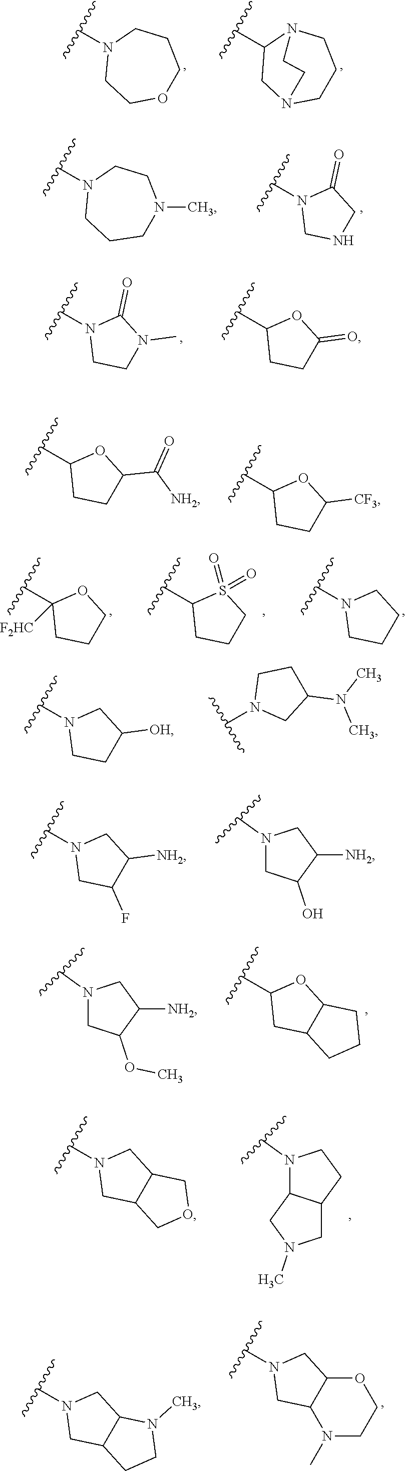

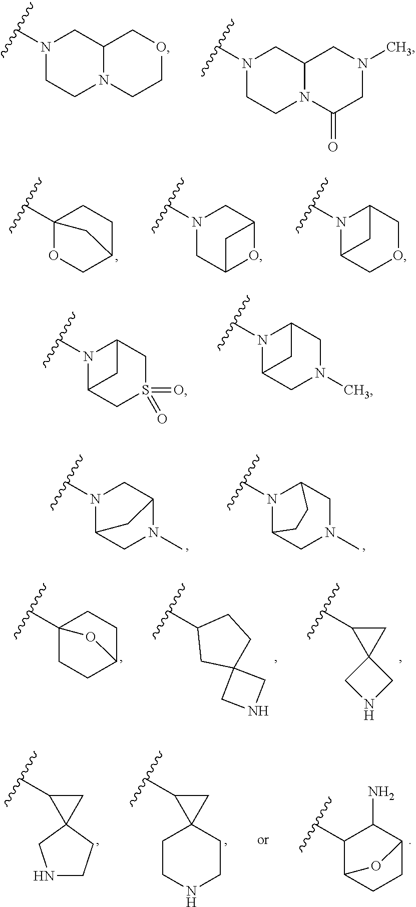

In some embodiments of Formula AI and subformula thereof, W is optionally substituted 3 to 8-membered cycloalkyl.

In some embodiments, W is

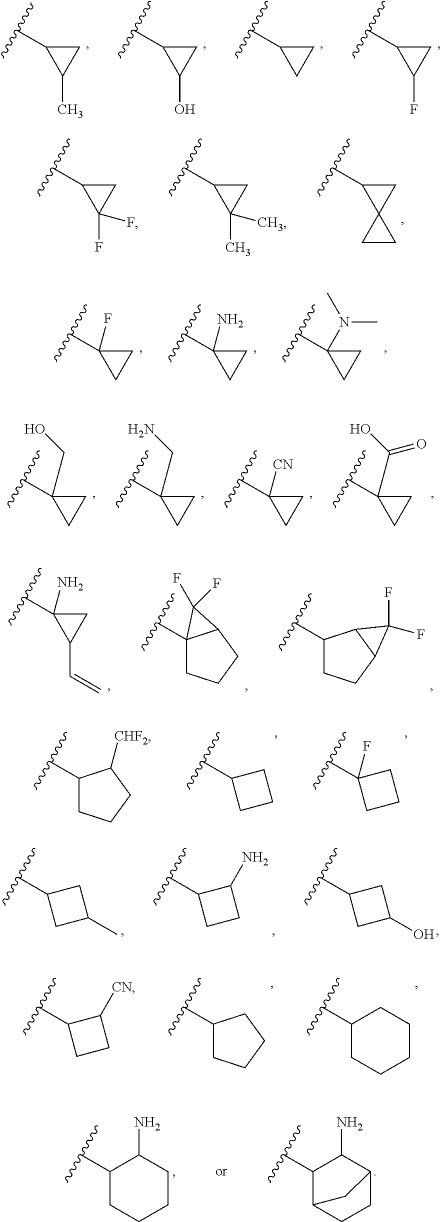

In some embodiments of Formula AI and subformula thereof, W is optionally substituted 3 to 8-membered heteroaryl.

In some embodiments, W is

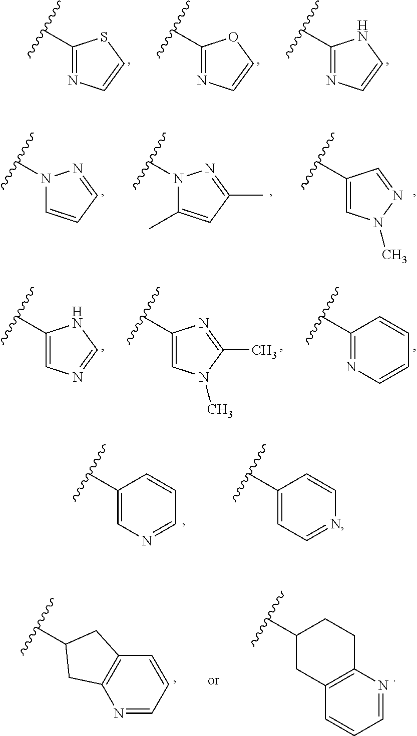

In some embodiments of Formula AI and subformula thereof, W is optionally substituted 6- to 10-membered aryl (e.g., phenyl, 4-hydroxy-phenyl, or 2,4-methoxy-phenyl).

In some embodiments, the RAS(ON) inhibitor is selected from Table A1, or a pharmaceutically acceptable salt or stereoisomer thereof. In some embodiments, the RAS(ON) inhibitor is selected from Table A1, or a pharmaceutically acceptable salt or atropisomer thereof.

TABLE A1
Certain Compounds of the Present Invention

| Ex# | Structure |
|---|---|
| AA1 | 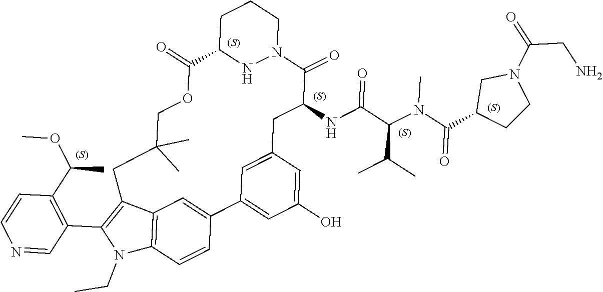 |
| AA2 | 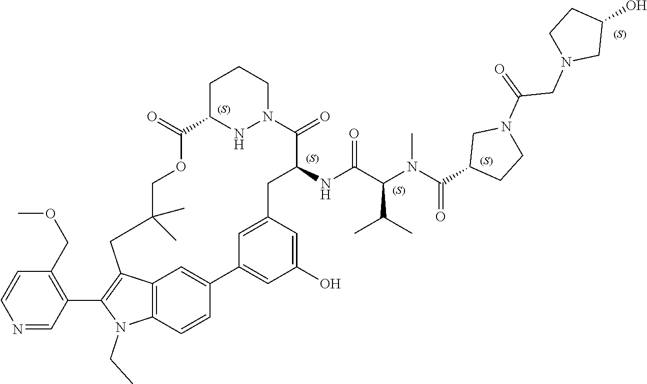 |
| AA3 | 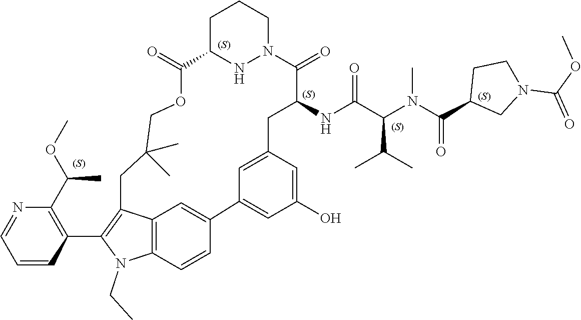 |
| AA4 | 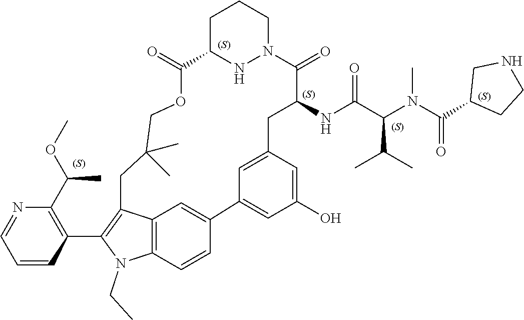 |
| AA5 | 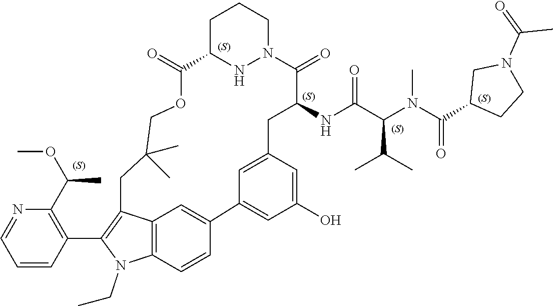 |
| AA6 | 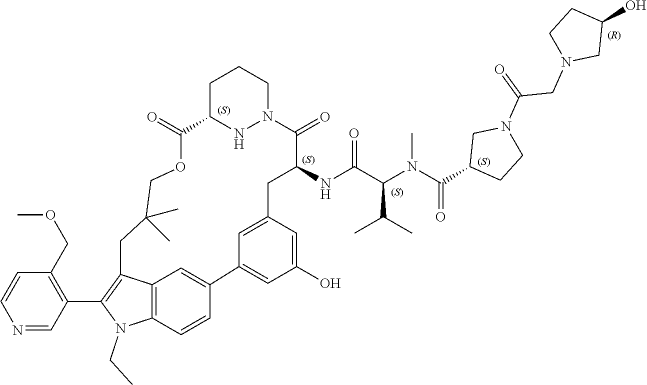 |
| AA7 | 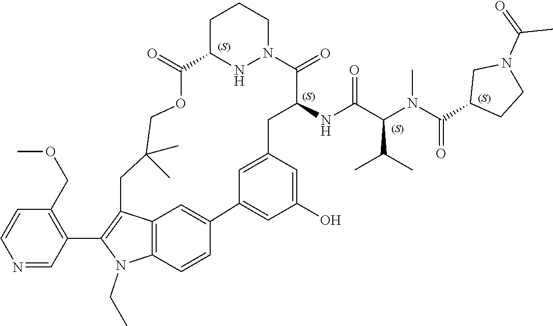 |
| AA8 | 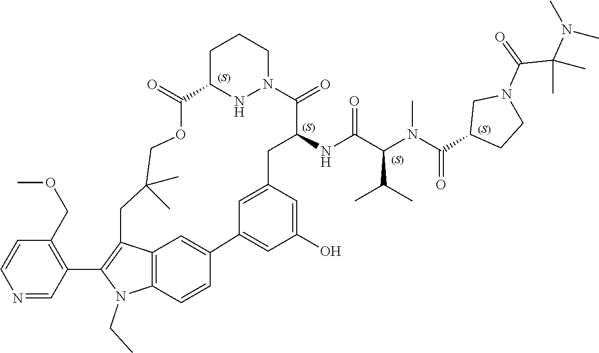 |
| AA9 | 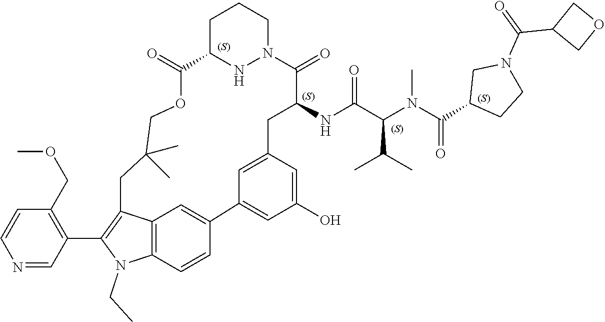 |
| AA10 | 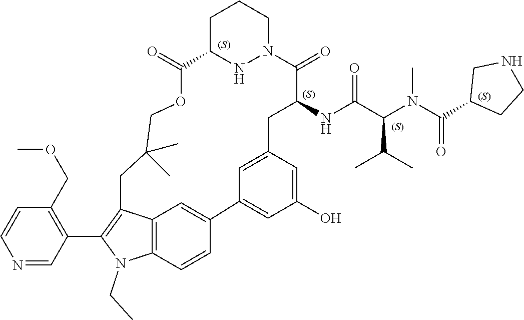 |
| AA11 | 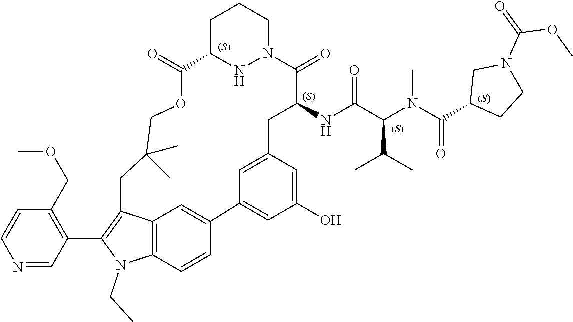 |
| AA12 | 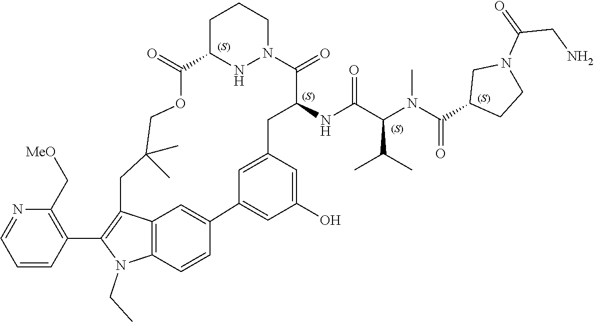 |
| AA13 | 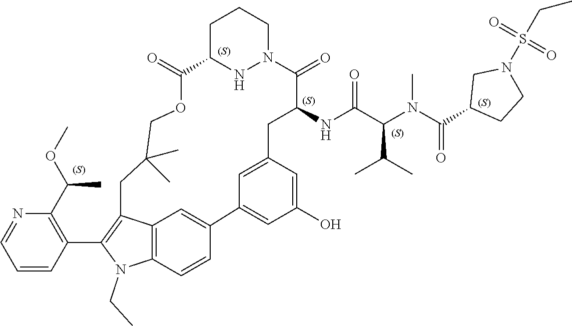 |
| AA14 | 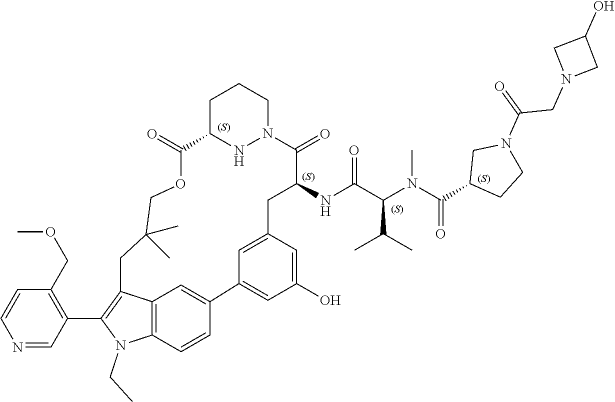 |
| AA15 | 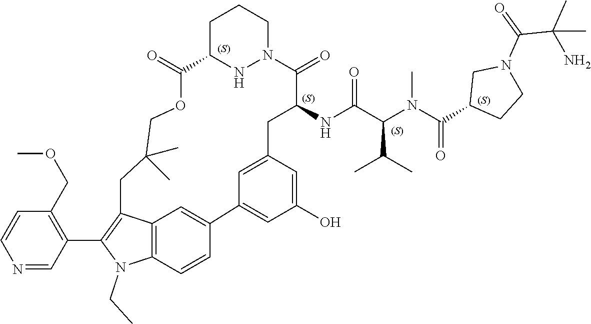 |
| AA16 | 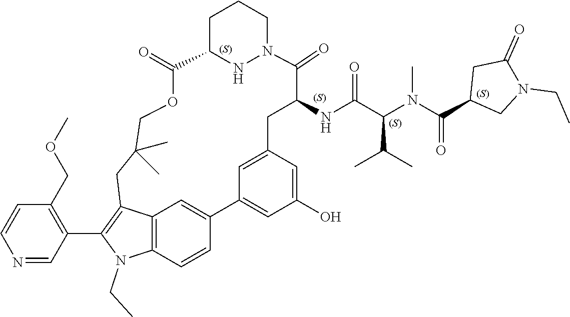 |
| AA17 | 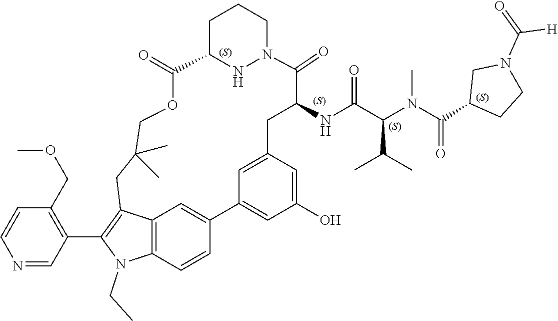 |
| AA18 | 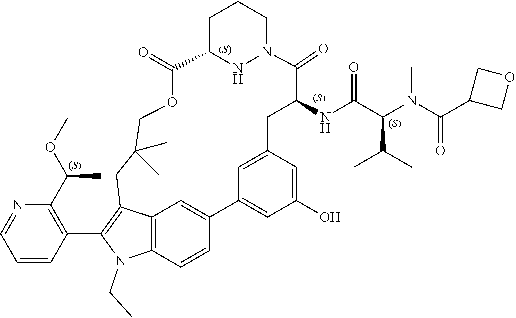 |
| AA19 | 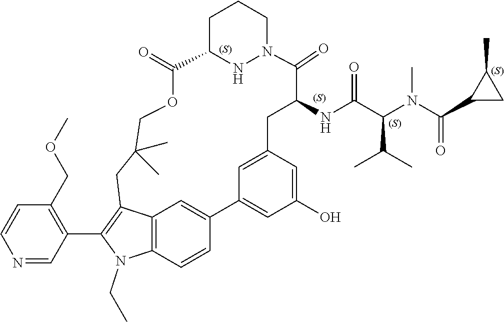 |
| AA20 | 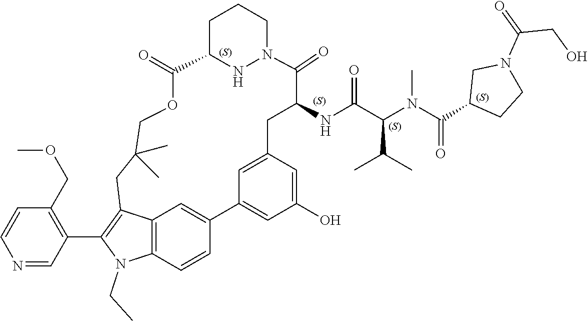 |
| AA21 | 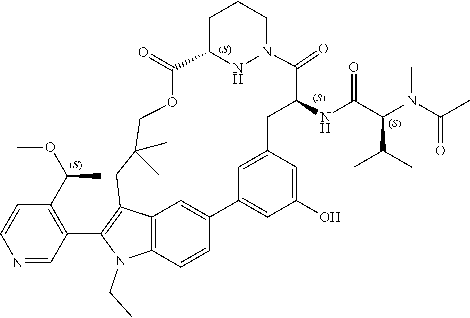 |
| AA22 | 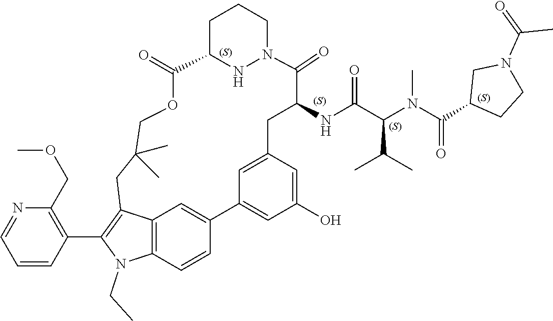 |
| AA23 | 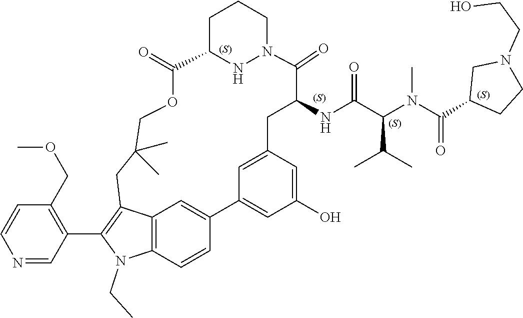 |
| AA24 | 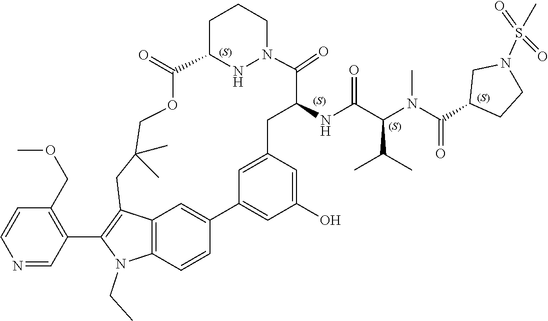 |
| AA25 | 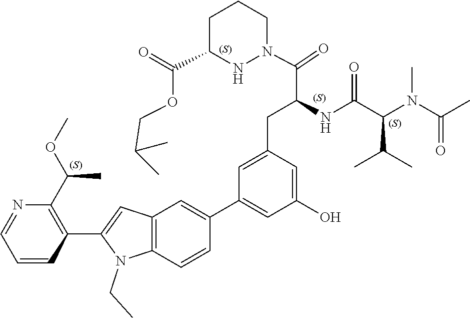 |
| AA26 | 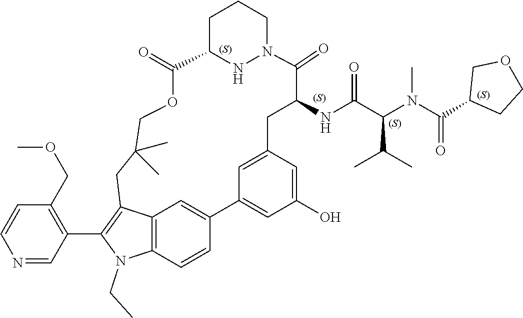 |
| AA27 | 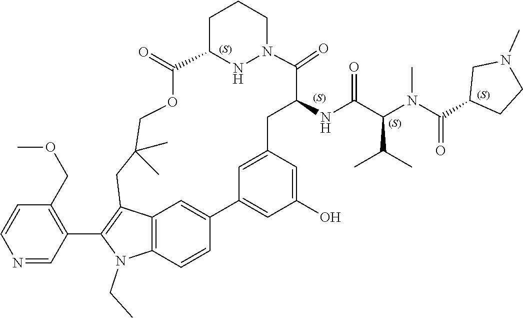 |
| AA28 | 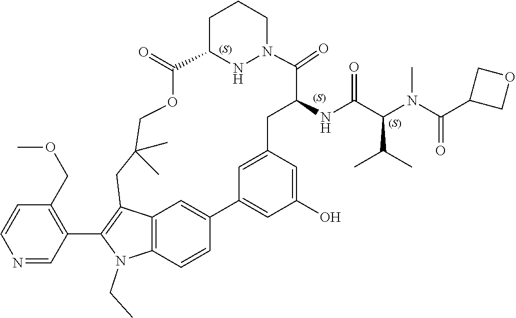 |
| AA29 | 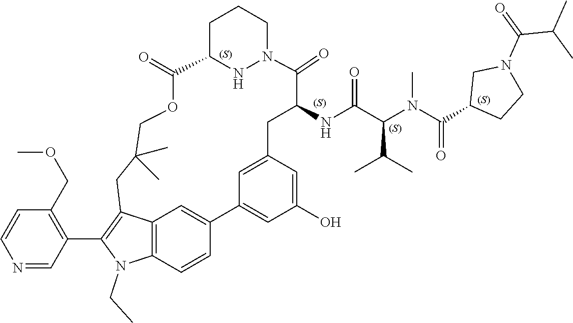 |
| AA30 | 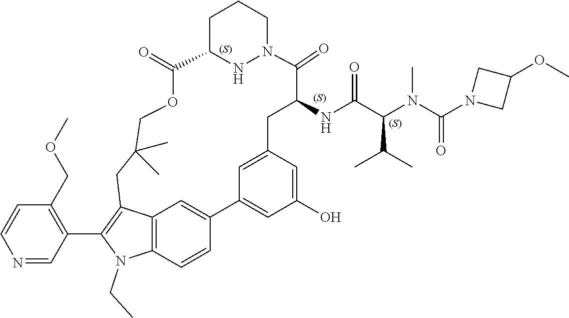 |
| AA31 | 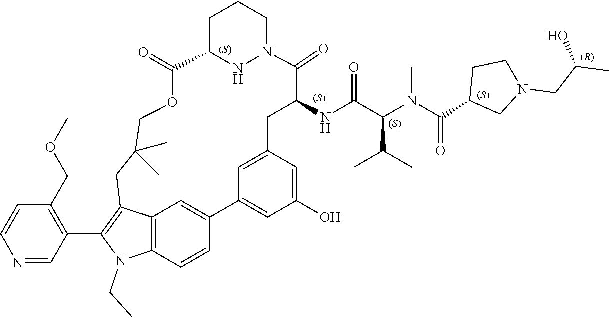 |
| AA32 | 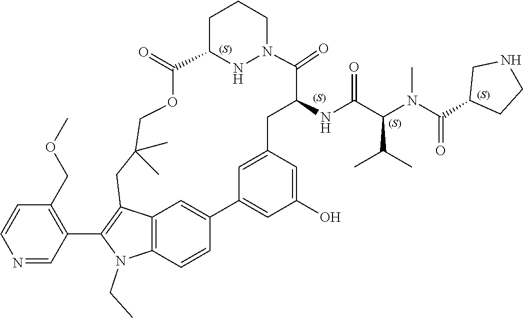 |
| AA33 | 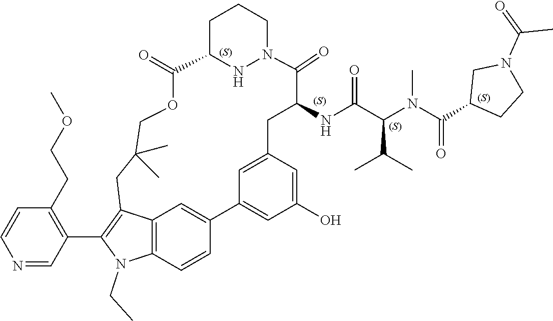 |
| AA34 | 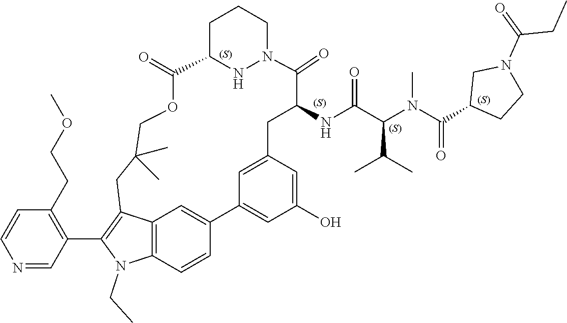 |
| AA35 | 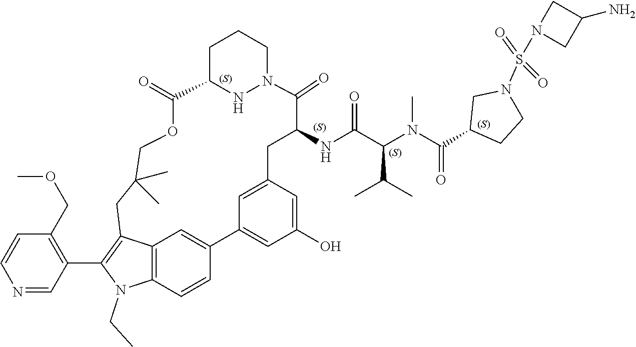 |
| AA36 | 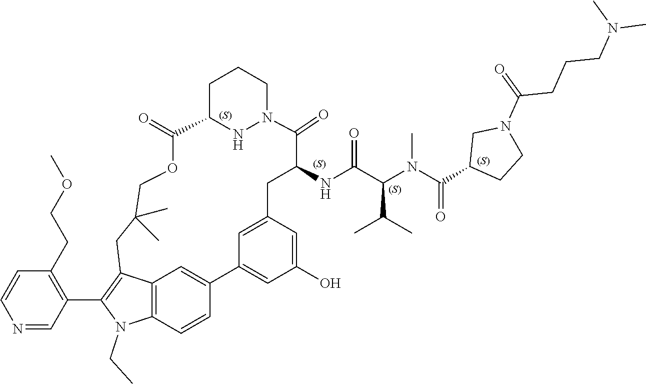 |
| AA37 | 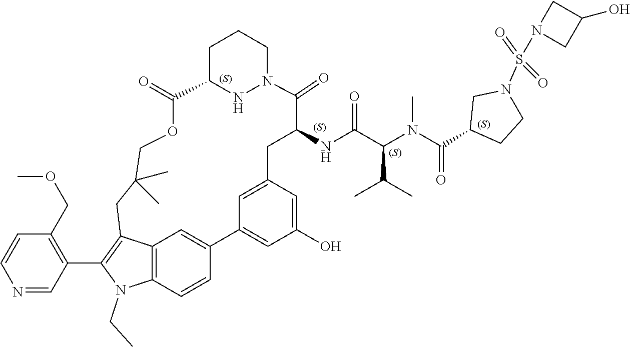 |
| AA38 | 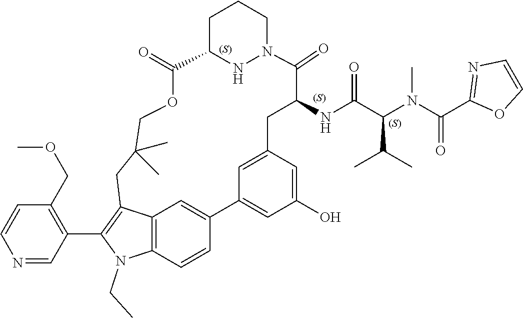 |
| AA39 | 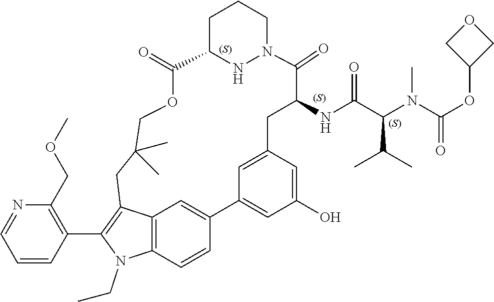 |
| AA40 | 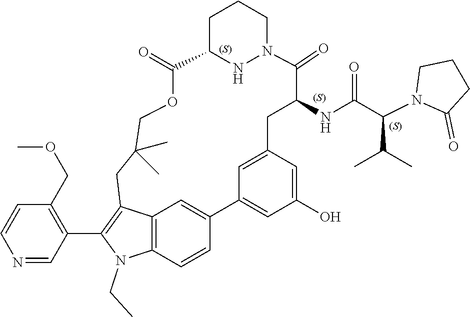 |
| AA41 | 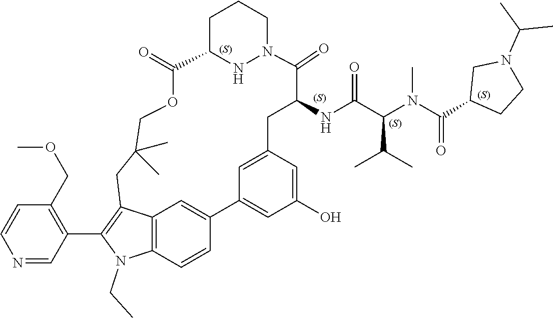 |
| AA42 | 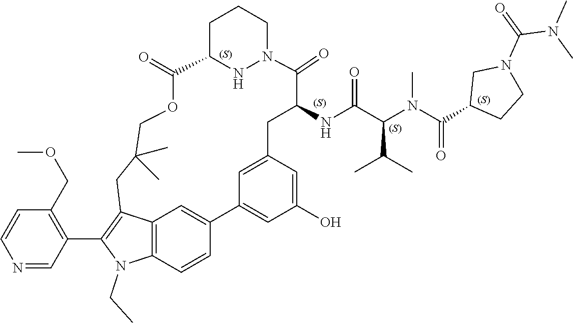 |
| AA43 | 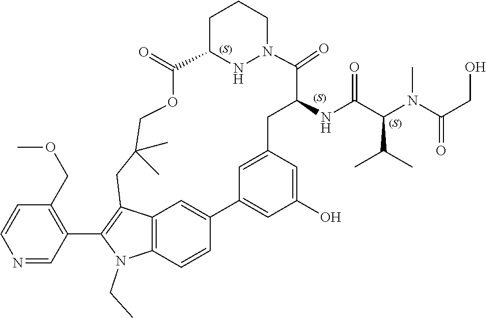 |
| AA44 | 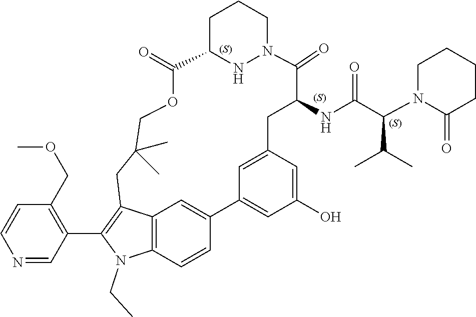 |
| AA45 | 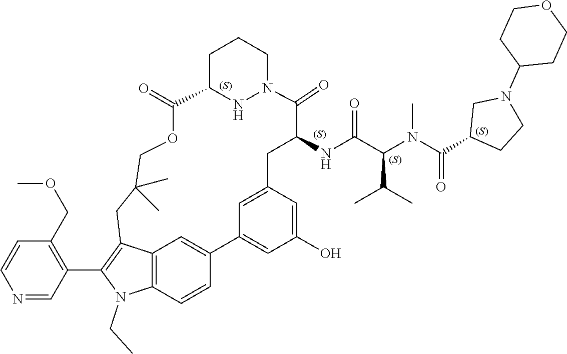 |
| AA46 | 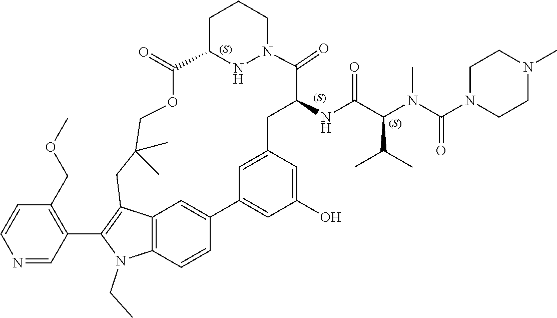 |
| AA47 | 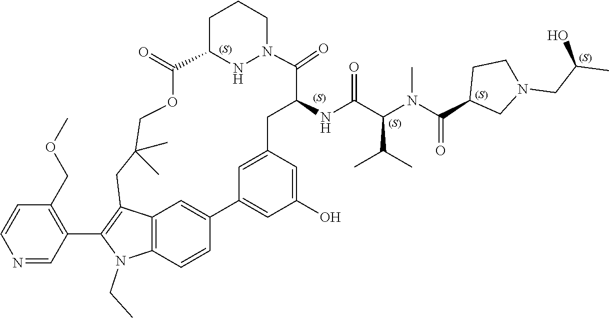 |
| AA48 | 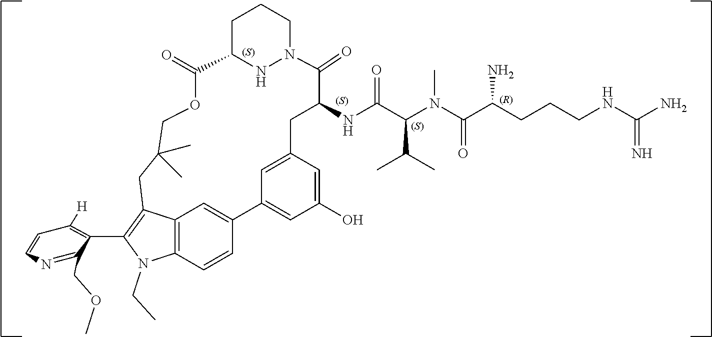 |
| AA49 | 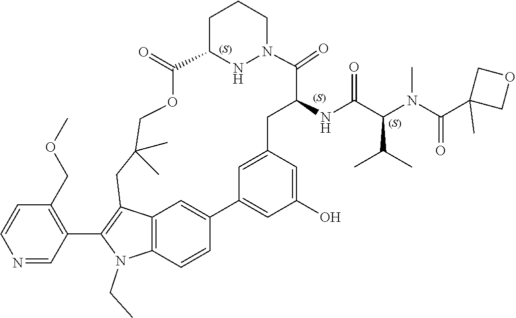 |
| AA50 | 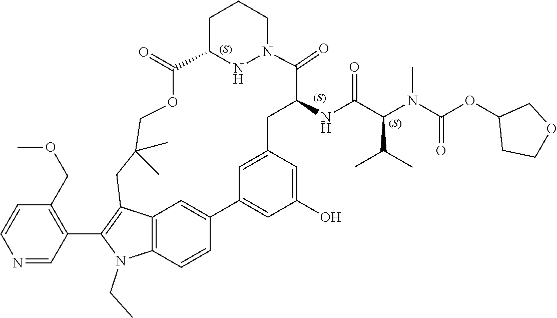 |
| AA51 | 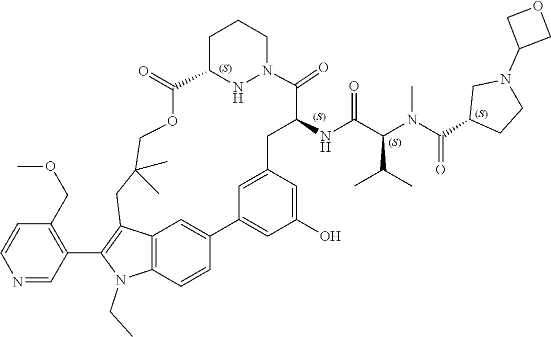 |
| AA52 | 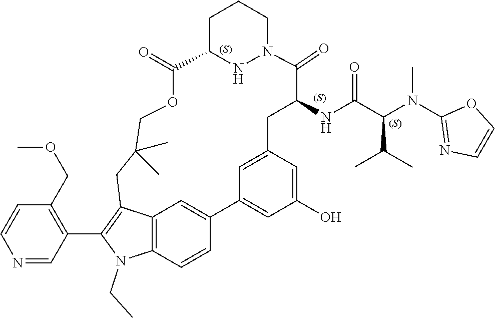 |
| AA53 | 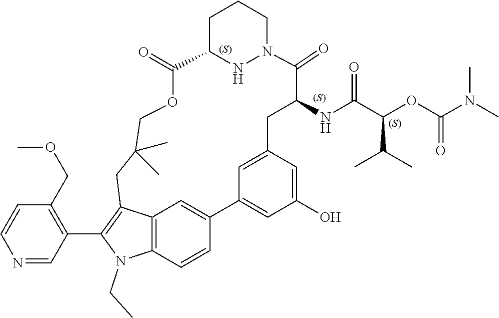 |
| AA54 | 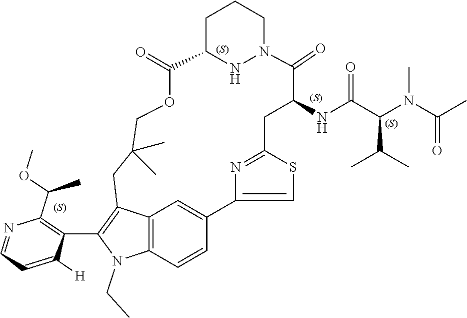 |
| AA55 | 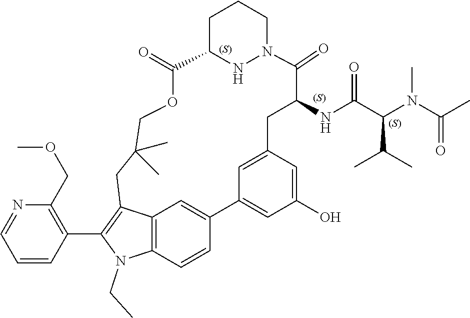 |
| AA56 | 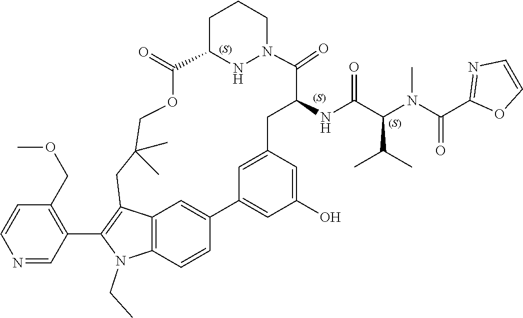 |
| AA57 | 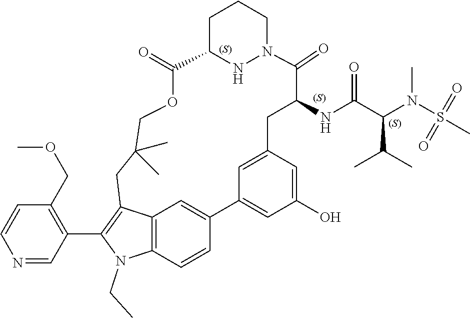 |
| AA58 | 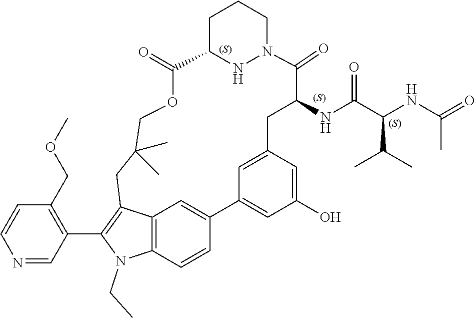 |
| AA59 | 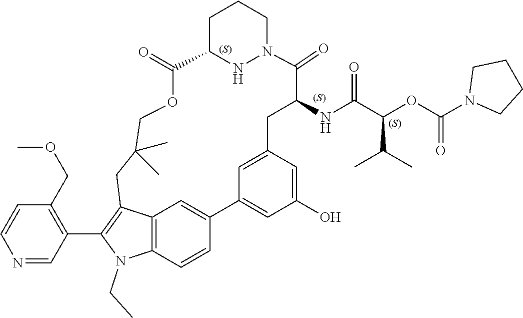 |
| AA60 | 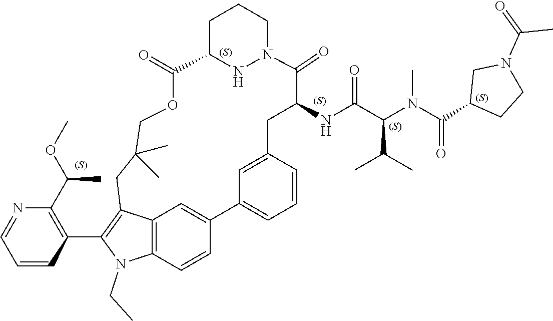 |
| AA61 | 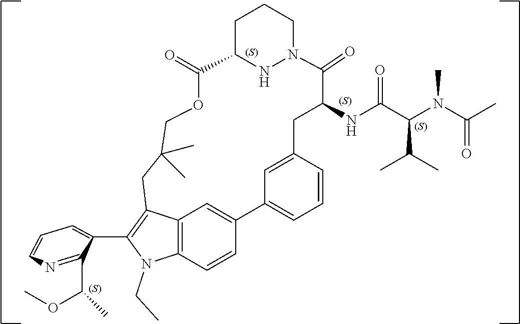 |
| AA62 | 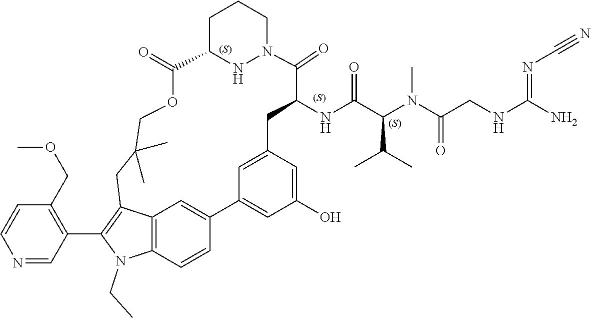 |
| AA63 | 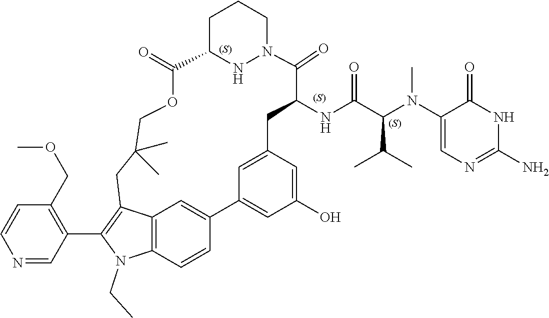 |
| AA64 | 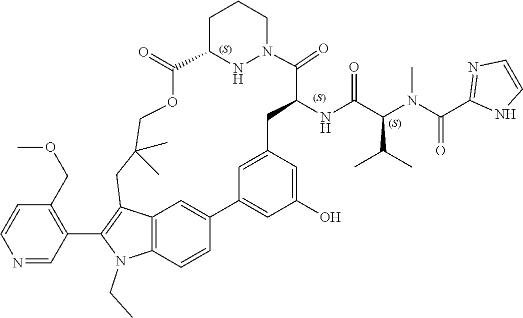 |
| AA65 | 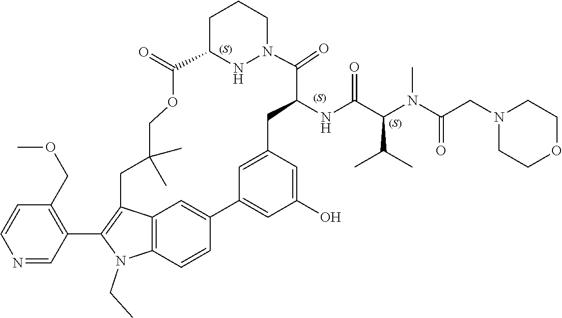 |
| AA66 | 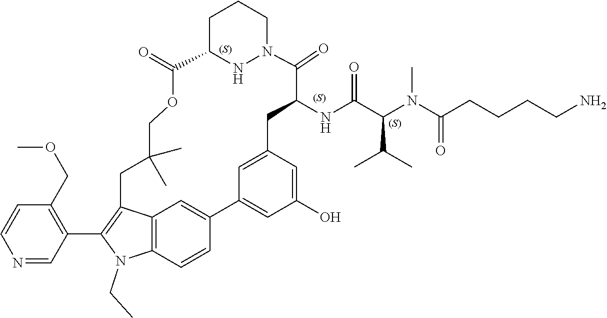 |
| AA67 | 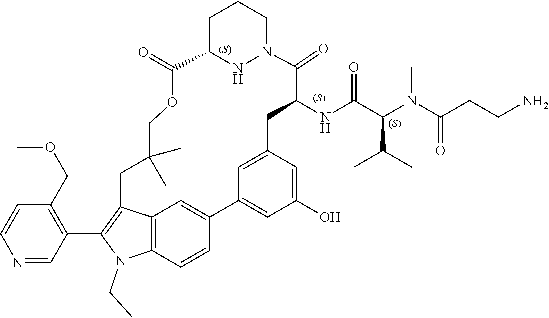 |
| AA68 | 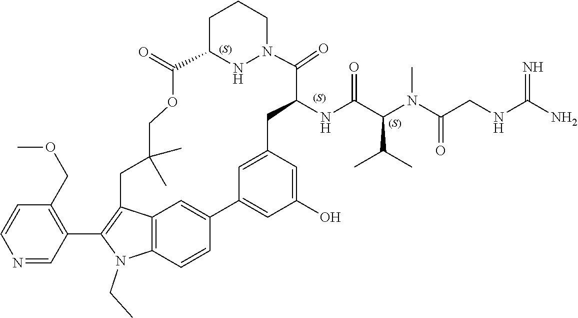 |
| AA69 | 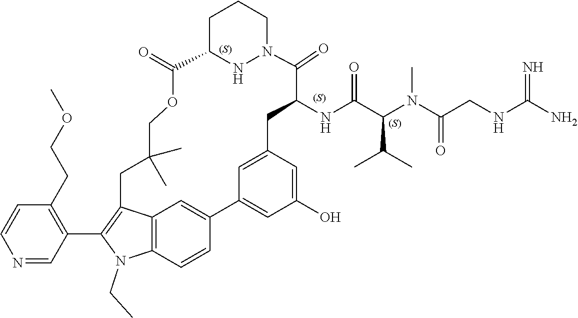 |
| AA70 | 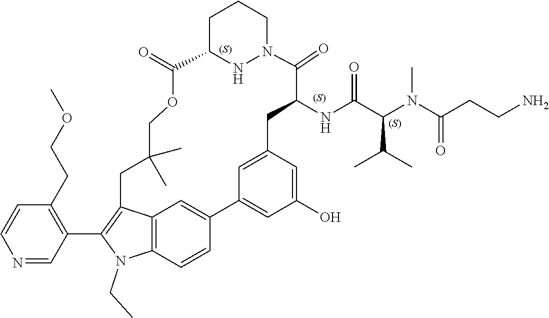 |
| AA71 | 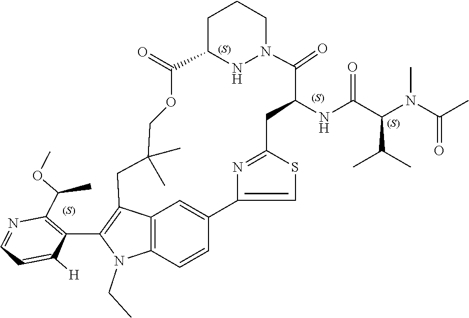 |
| AA72 | 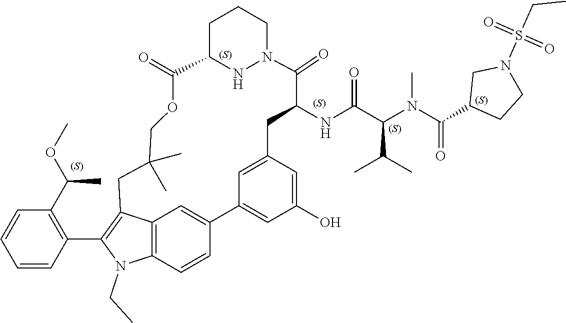 |
| AA73 | 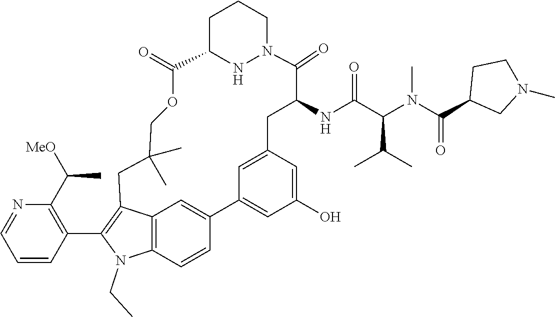 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA74 | 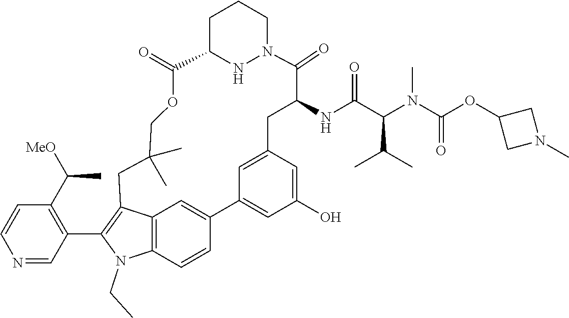 [SMILES: CCn1c(-c2cnccc2[C@H](C)OC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA75 | 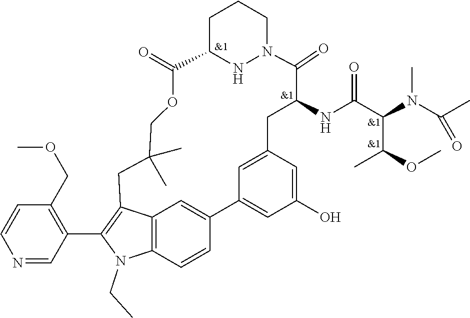 [SMILES: CCn1c(-c2cnccc2COC)c2c3cc(ccc31)-c1cc(O)cc(c1)C[C@H](NC(=O)[C@H]([C@H](C)OC)N(C)C(C)=O)C(=O)N1CCC[C@H](N1)C(=O)OCC(C)(C)C2] |
| AA76 | 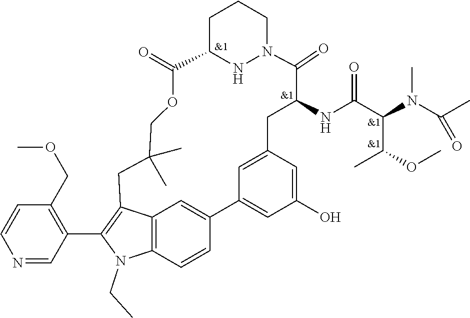 [SMILES: CCn1c(-c2cnccc2COC)c2c3cc(ccc31)-c1cc(O)cc(c1)C[C@H](NC(=O)[C@H]([C@@H](C)OC)N(C)C(C)=O)C(=O)N1CCC[C@H](N1)C(=O)OCC(C)(C)C2] |
| AA77 | 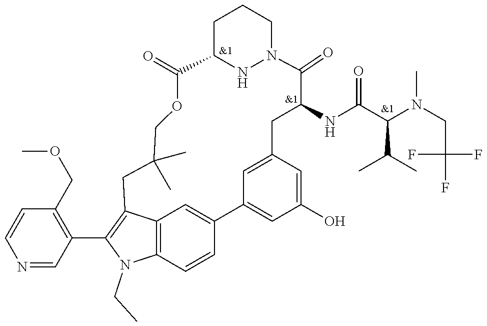 [SMILES: CCn1c(-c2cnccc2COC)c2c3cc(ccc31)-c1cc(O)cc(c1)C[C@H](NC(=O)[C@H](C(C)C)N(C)CC(F)(F)F)C(=O)N1CCC[C@H](N1)C(=O)OCC(C)(C)C2] |
| AA78 | 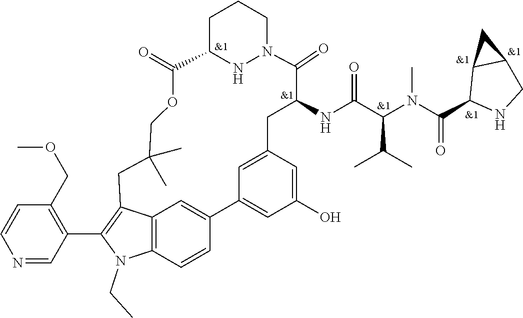 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NC[C@@H]3C[C@@H]31)C(=O)N1CCC[C@H](N1)C(=O)OCC(C)(C)C2] |
| AA79 | 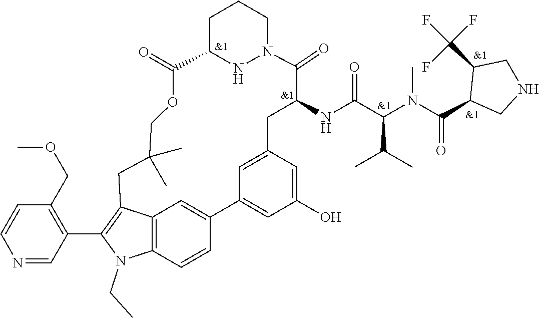 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1C(F)(F)F)C(=O)N1CCC[C@H](N1)C(=O)OCC(C)(C)C2] |
| AA80 | 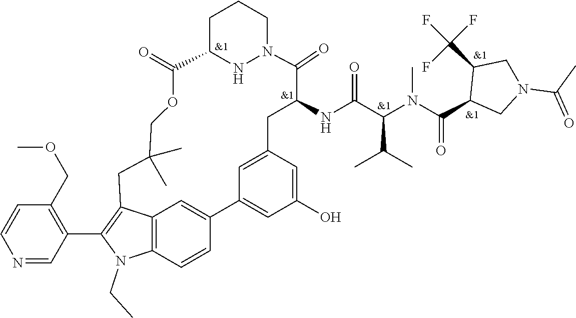 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CN(C(C)=O)C[C@@H]1C(F)(F)F)C(=O)N1CCC[C@H](N1)C(=O)OCC(C)(C)C2] |
| AA81 | 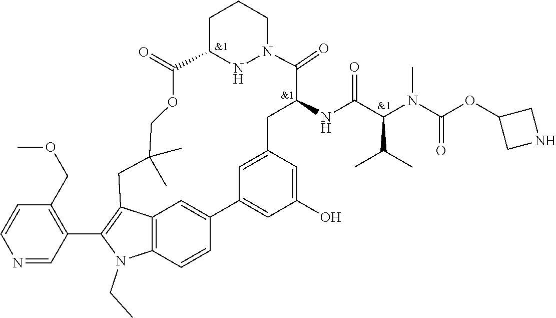 [SMILES: CCn1c(-c2cnccc2COC)c2c3cc(ccc31)-c1cc(O)cc(c1)C[C@H](NC(=O)[C@H](C(C)C)N(C)C(=O)OC1CNC1)C(=O)N1CCC[C@H](N1)C(=O)OCC(C)(C)C2] |
| AA82 | 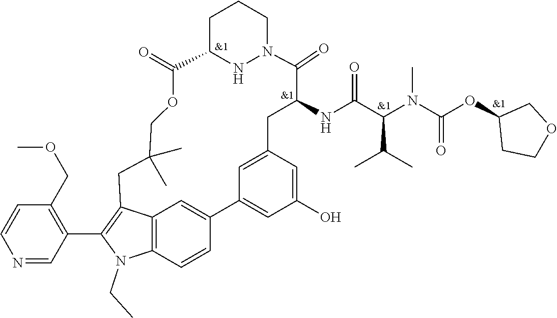 [SMILES: CCn1c(-c2cnccc2COC)c2c3cc(ccc31)-c1cc(O)cc(c1)C[C@H](NC(=O)[C@H](C(C)C)N(C)C(=O)O[C@@H]1CCOC1)C(=O)N1CCC[C@H](N1)C(=O)OCC(C)(C)C2] |
| AA83 | 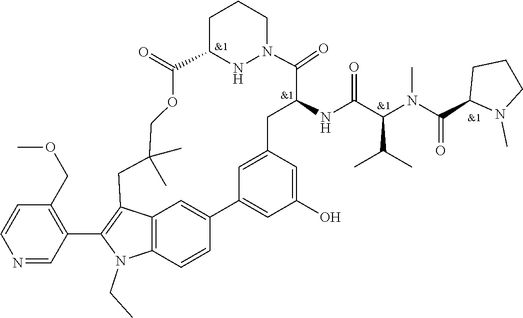 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCN1C)C(=O)N1CCC[C@H](N1)C(=O)OCC(C)(C)C2] |
| AA84 | 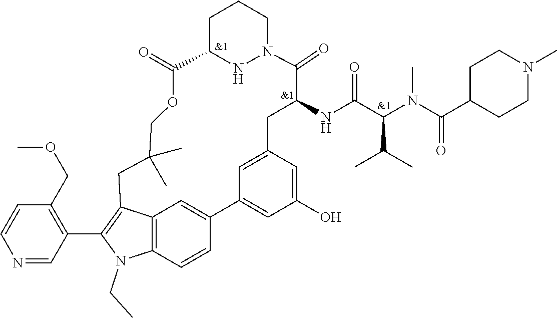 [SMILES: CCn1c(-c2cnccc2COC)c2c3cc(ccc31)-c1cc(O)cc(c1)C[C@H](NC(=O)[C@H](C(C)C)N(C)C(=O)C1CCN(C)CC1)C(=O)N1CCC[C@H](N1)C(=O)OCC(C)(C)C2] |
| AA85 | 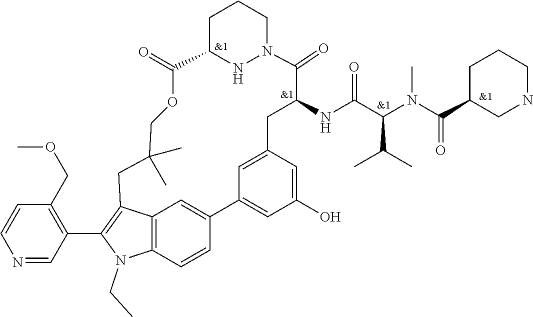 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCNC1)C(=O)N1CCC[C@H](N1)C(=O)OCC(C)(C)C2] |
| AA86 | 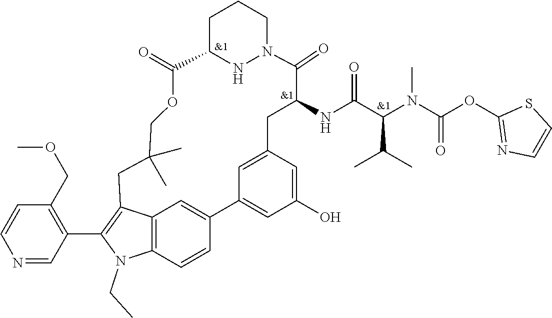 [SMILES: CCn1c(-c2cnccc2COC)c2c3cc(ccc31)-c1cc(O)cc(c1)C[C@H](NC(=O)[C@H](C(C)C)N(C)C(=O)Oc1nccs1)C(=O)N1CCC[C@H](N1)C(=O)OCC(C)(C)C2] |
| AA87 | 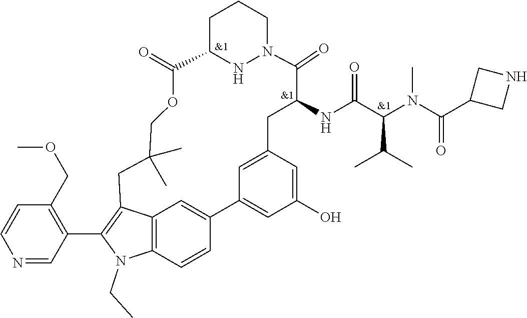 [SMILES: CCn1c(-c2cnccc2COC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA88 | 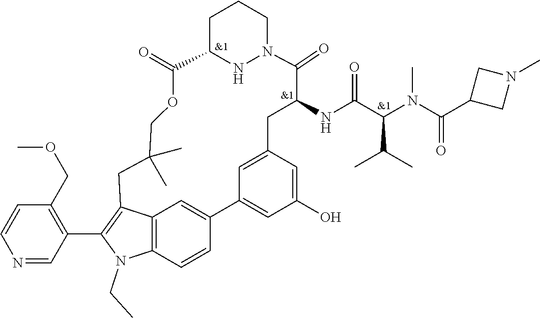 [SMILES: CCn1c(-c2cnccc2COC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA89 | 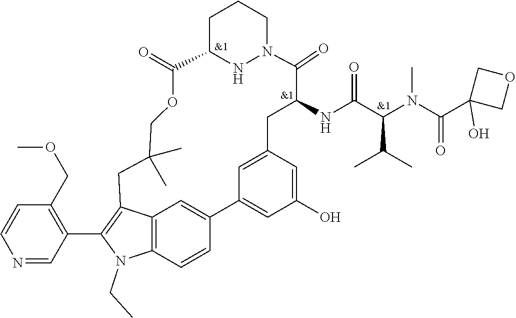 [SMILES: CCn1c(-c2cnccc2COC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AA90 | 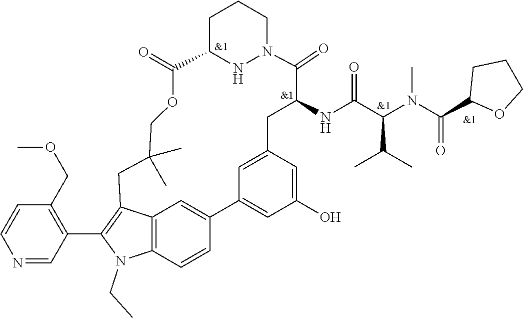 |
| AA91 | 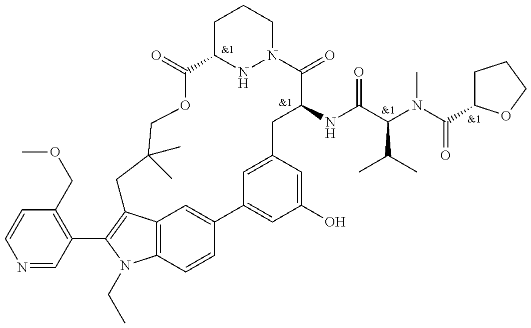 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCO1)C(=O)N1CCC[C@H](N1)C(=O)OCC(C)(C)C2] |
| AA92 | 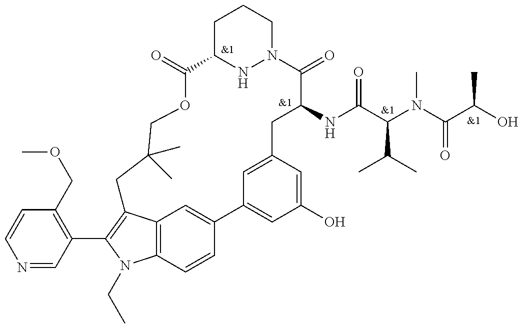 [SMILES: CCn1c(-c2cnccc2COC)c2c3cc(ccc31)-c1cc(O)cc(c1)C[C@H](NC(=O)[C@H](C(C)C)N(C)C(=O)[C@@H](C)O)C(=O)N1CCC[C@H](N1)C(=O)OCC(C)(C)C2] |
| AA93 | 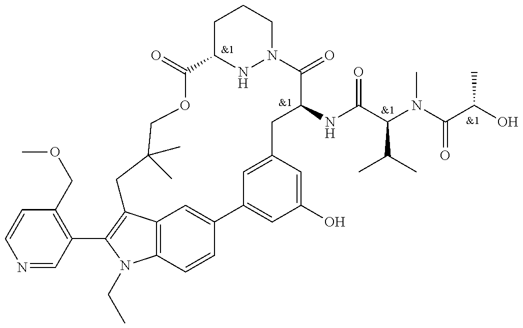 [SMILES: CCn1c(-c2cnccc2COC)c2c3cc(ccc31)-c1cc(O)cc(c1)C[C@H](NC(=O)[C@H](C(C)C)N(C)C(=O)[C@H](C)O)C(=O)N1CCC[C@H](N1)C(=O)OCC(C)(C)C2] |
| AA94 | 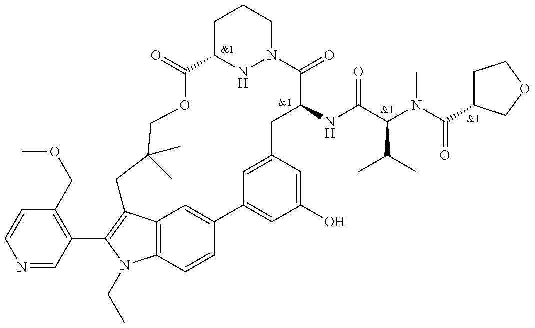 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCOC1)C(=O)N1CCC[C@H](N1)C(=O)OCC(C)(C)C2] |
| AA95 | 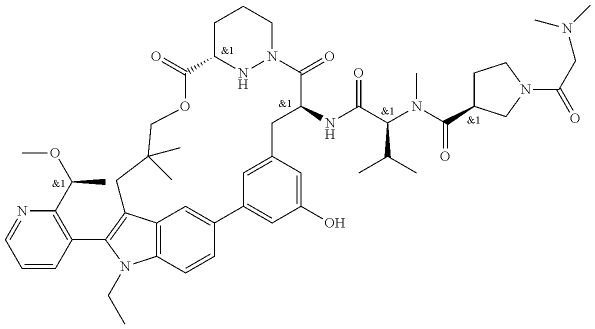 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA96 | 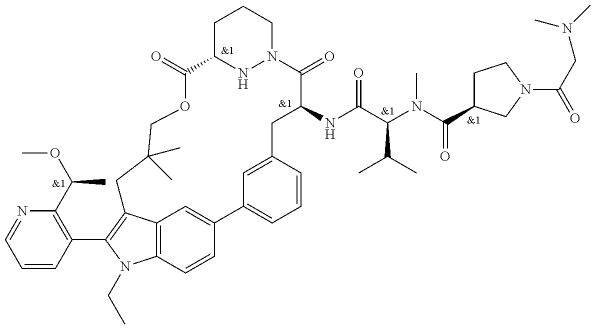 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA97 | 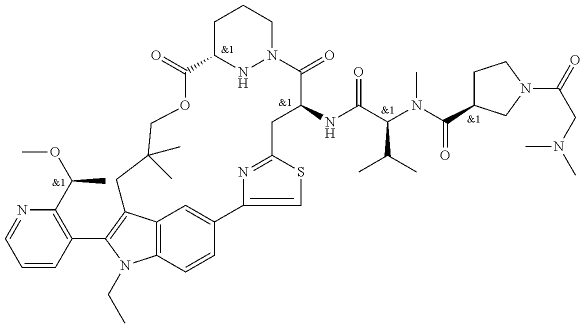 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA98 | 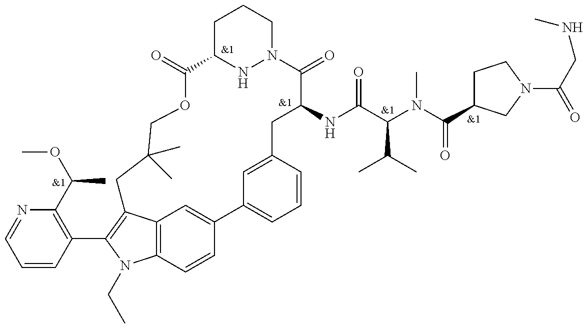 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA99 | 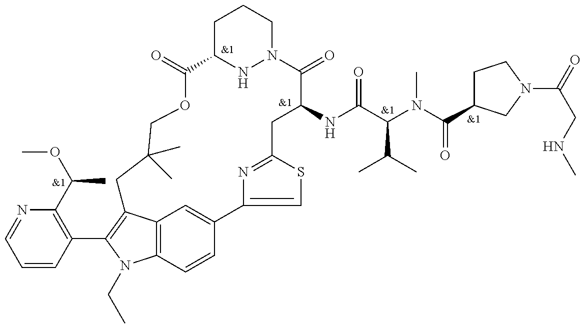 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA100 | 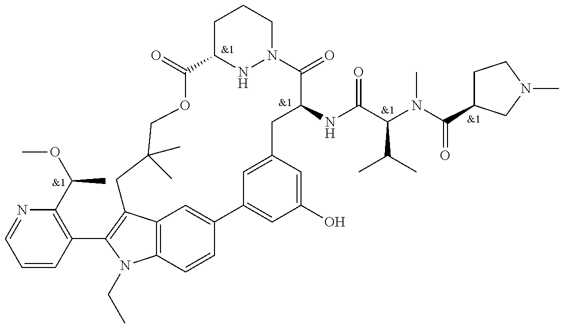 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA101 | 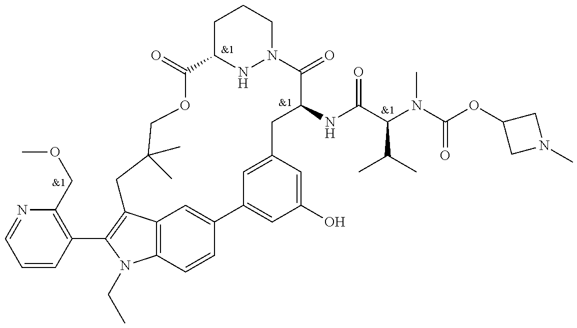 [SMILES: CCn1c(-c2cccnc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA102 | 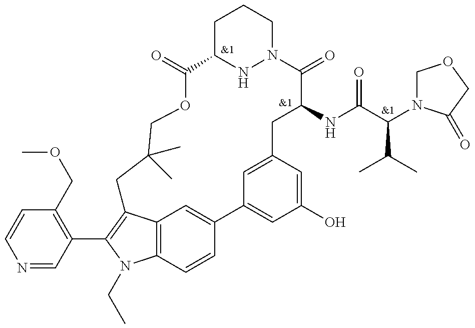 [SMILES: CCn1c(-c2cnccc2COC)c2c3cc(ccc31)-c1cc(O)cc(c1)C[C@H](NC(=O)[C@H](C(C)C)N1COCC1=O)C(=O)N1CCC[C@H](N1)C(=O)OCC(C)(C)C2] |
| AA103 | 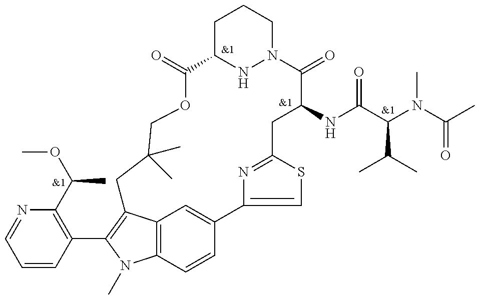 [SMILES: CO[C@@H](C)c1ncccc1-c1c2c3cc(ccc3n1C)-c1csc(n1)C[C@H](NC(=O)[C@H](C(C)C)N(C)C(C)=O)C(=O)N1CCC[C@H](N1)C(=O)OCC(C)(C)C2] |
| AA104 | 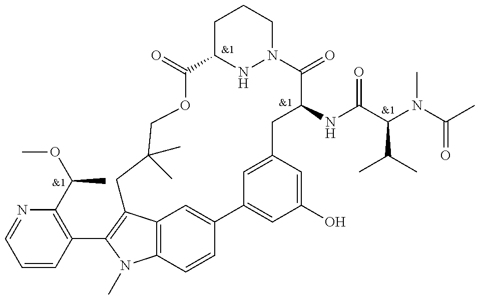 [SMILES: CO[C@@H](C)c1ncccc1-c1c2c3cc(ccc3n1C)-c1cc(O)cc(c1)C[C@H](NC(=O)[C@H](C(C)C)N(C)C(C)=O)C(=O)N1CCC[C@H](N1)C(=O)OCC(C)(C)C2] |
| AA105 | 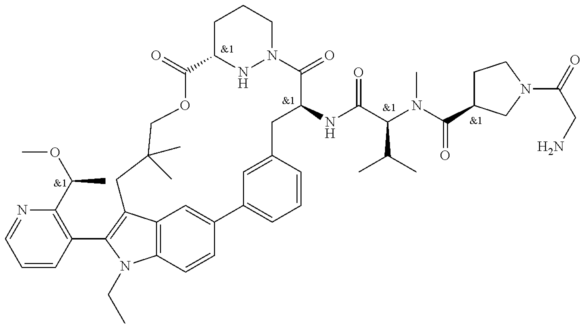 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA106 | 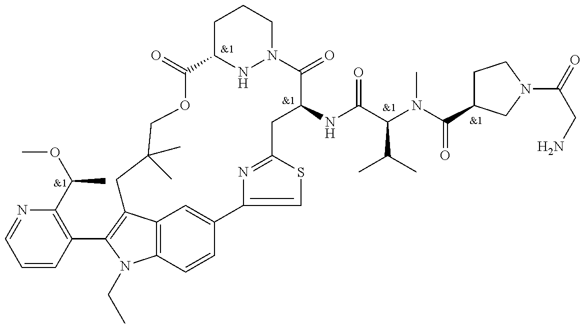 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA107 | 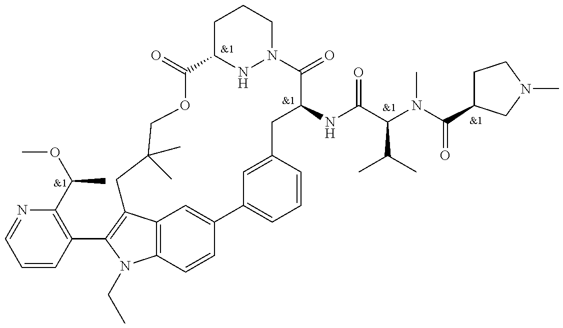 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA108 | 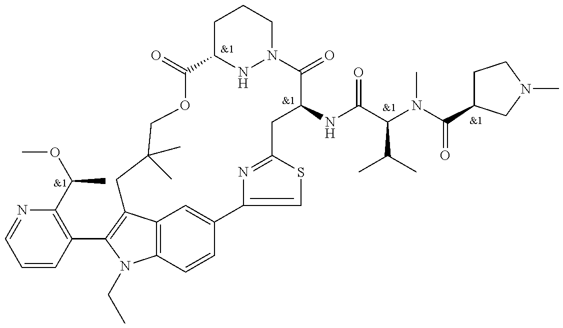 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C)C1)C(=O)N1CCC[C@H](N1)C(=O)OCC(C)(C)C2] |
| AA109 | 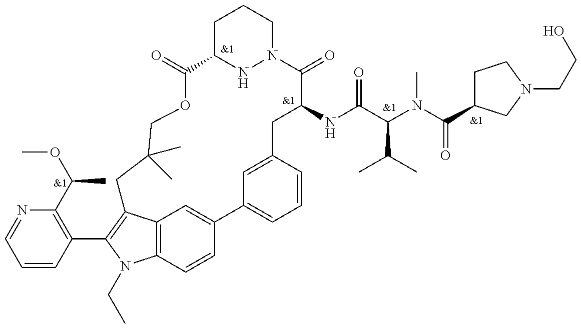 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA110 | 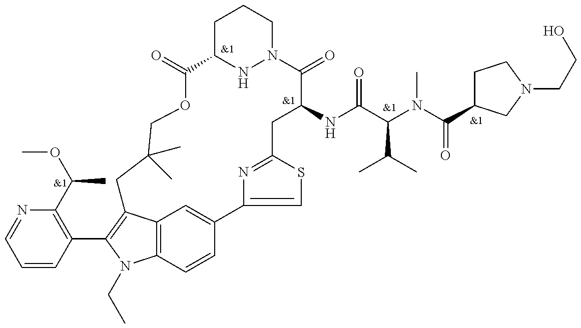 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA111 | 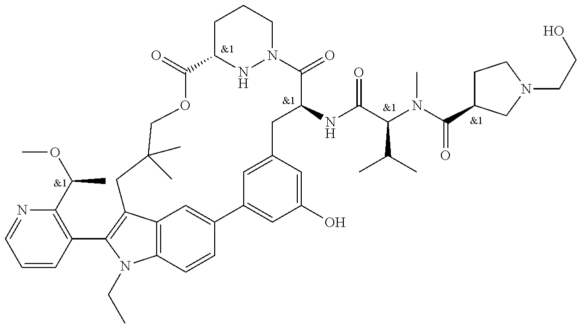 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA112 | 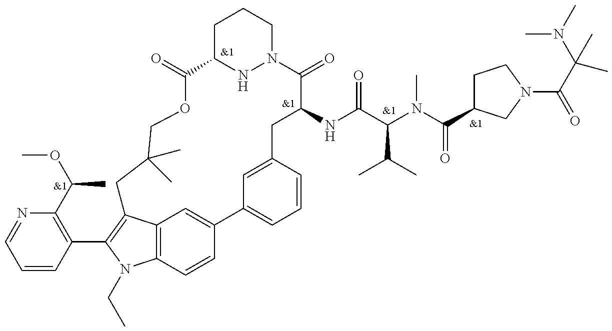 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA113 | 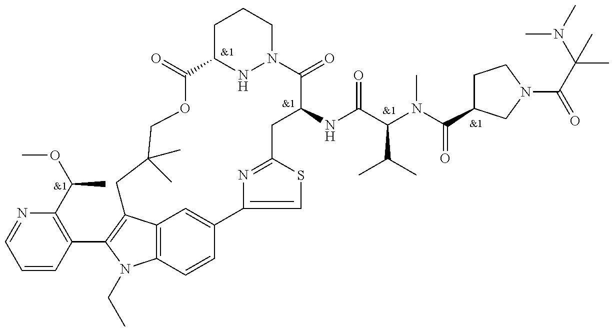 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA114 | 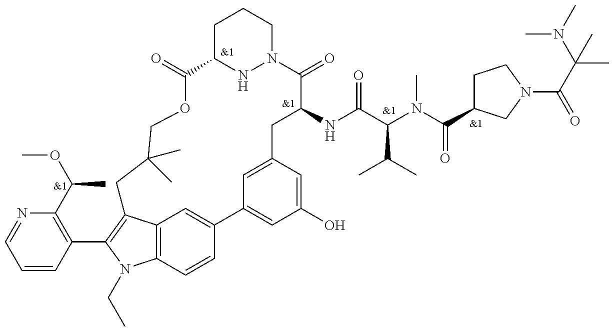 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA115 | 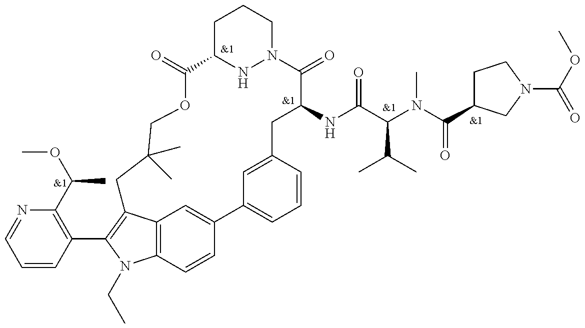 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA116 | 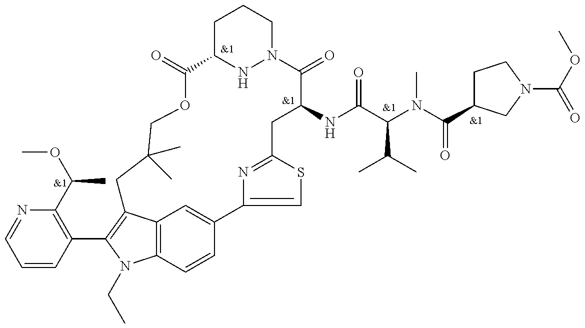 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA117 | 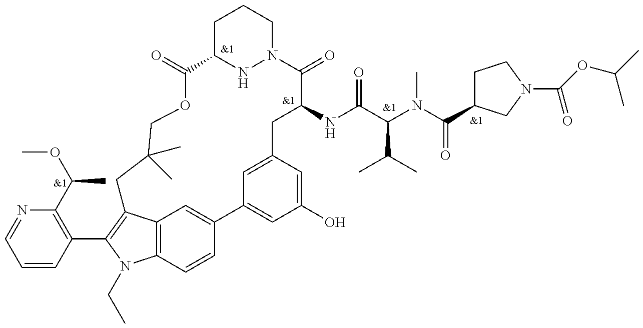 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)OC(C)C)C1)C(=O)N1CCC[C@H](N1)C(=O)OCC(C)(C)C2] |
| AA118 | 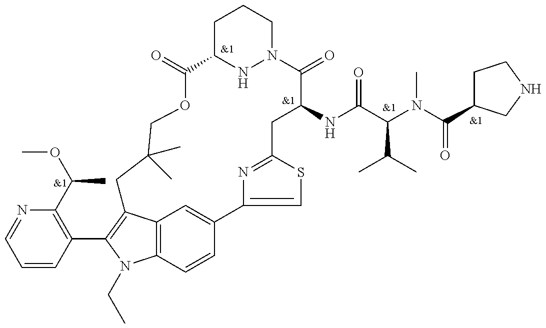 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCNC1)C(=O)N1CCC[C@H](N1)C(=O)OCC(C)(C)C2] |
| AA119 | 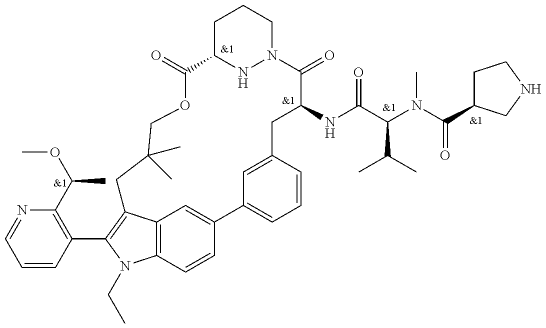 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCNC1)C(=O)N1CCC[C@H](N1)C(=O)OCC(C)(C)C2] |
| AA120 | 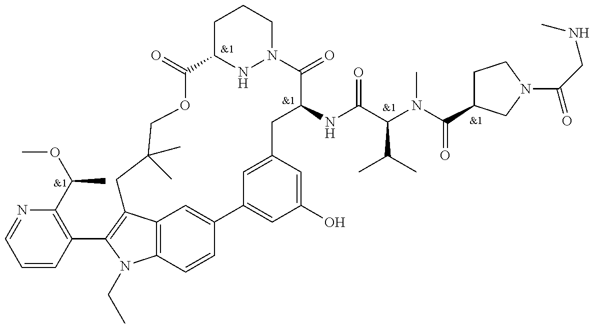 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA121 | 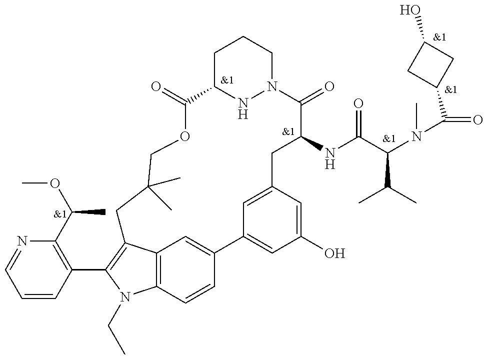 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H](O)C1)C(=O)N1CCC[C@H](N1)C(=O)OCC(C)(C)C2] |
| AA122 | 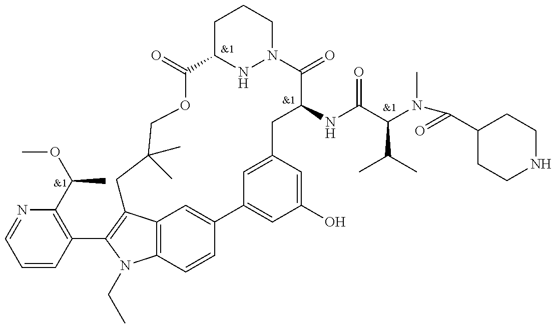 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNCC1)C(=O)N1CCC[C@H](N1)C(=O)OCC(C)(C)C2] |
| AA123 | 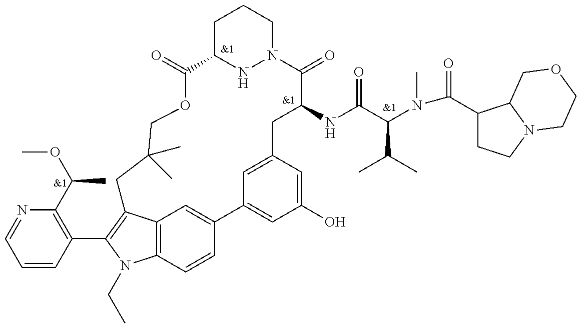 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCN3CCOCC13)C(=O)N1CCC[C@H](N1)C(=O)OCC(C)(C)C2] |
| AA124 | 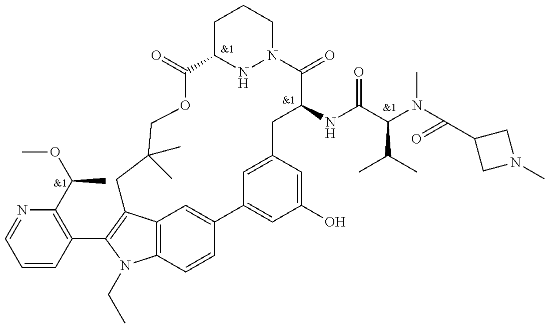 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA125 | 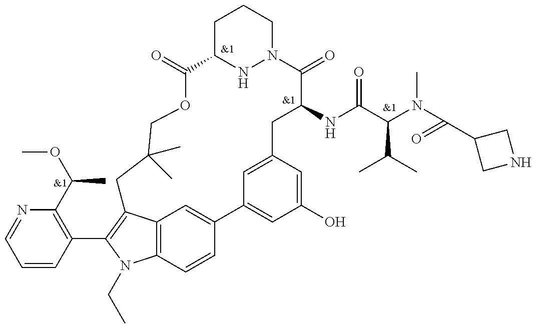 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA126 | 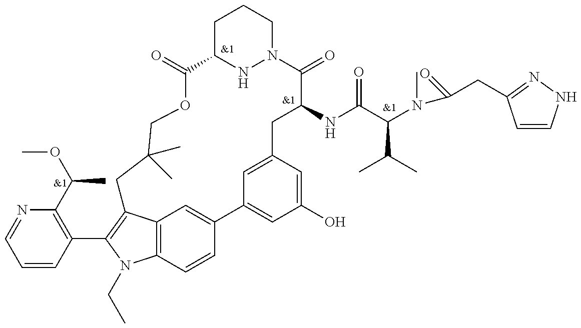 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1cc[nH]n1)C(=O)N1CCC[C@H](N1)C(=O)OCC(C)(C)C2] |
| AA127 | 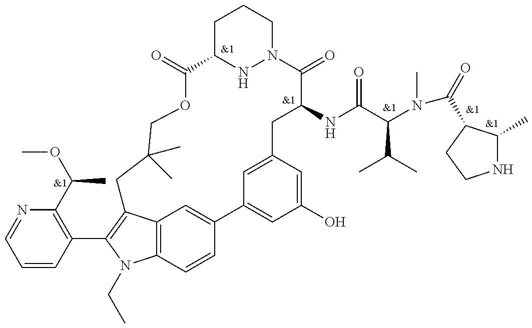 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN[C@H]1C)C(=O)N1CCC[C@H](N1)C(=O)OCC(C)(C)C2] |
| AA128 | 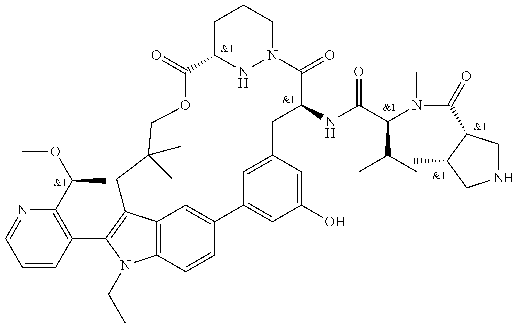 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1C)C(=O)N1CCC[C@H](N1)C(=O)OCC(C)(C)C2] |
| AA129 | 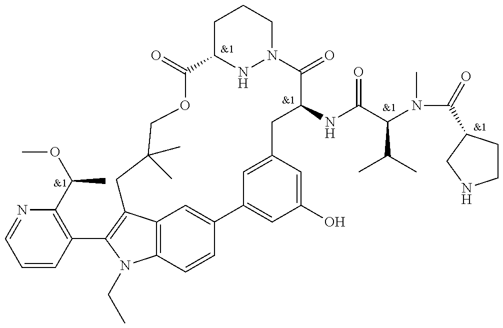 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CCNC1)C(=O)N1CCC[C@H](N1)C(=O)OCC(C)(C)C2] |
| AA130 | 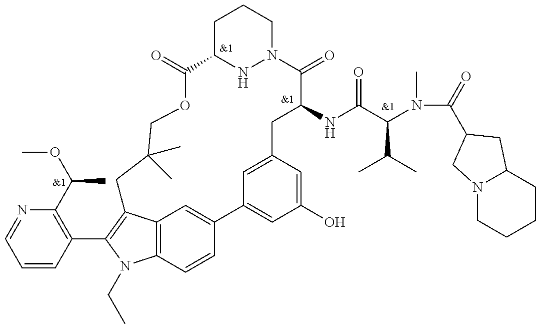 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CCCCN3C1)C(=O)N1CCC[C@H](N1)C(=O)OCC(C)(C)C2] |
| AA131 | 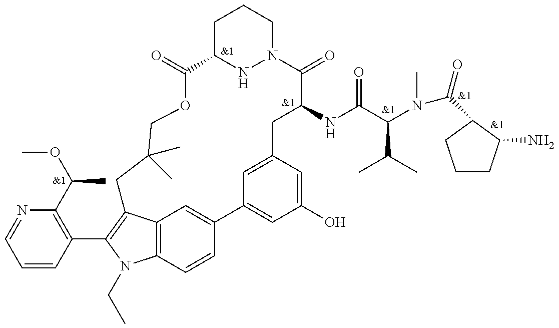 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCC[C@H]1N)C(=O)N1CCC[C@H](N1)C(=O)OCC(C)(C)C2] |
| AA132 | 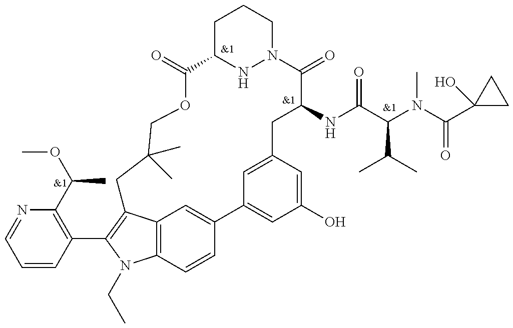 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)CC1)C(=O)N1CCC[C@H](N1)C(=O)OCC(C)(C)C2] |
| AA133 | 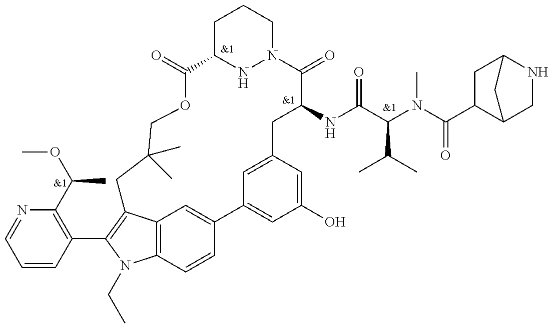 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CC1CN3)C(=O)N1CCC[C@H](N1)C(=O)OCC(C)(C)C2] |
| AA134 | 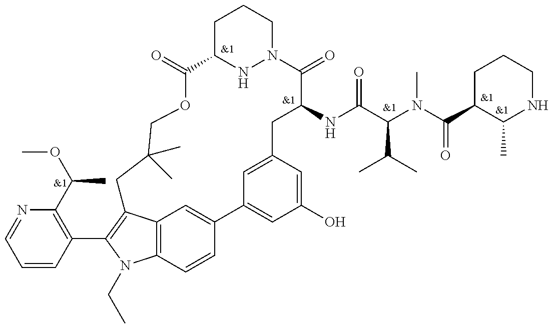 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCCN[C@@H]1C)C(=O)N1CCC[C@H](N1)C(=O)OCC(C)(C)C2] |
| AA135 | 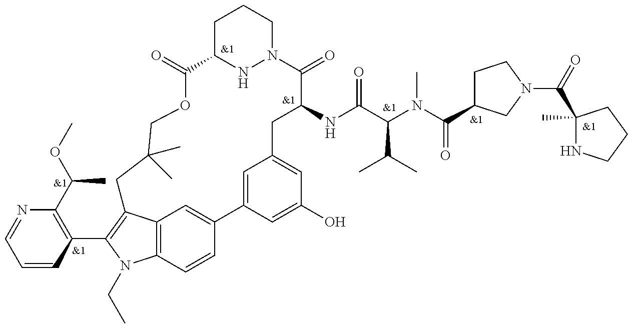 |
| AA136 | 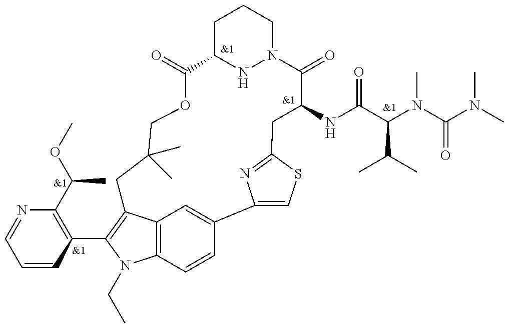 |
| AA137 | 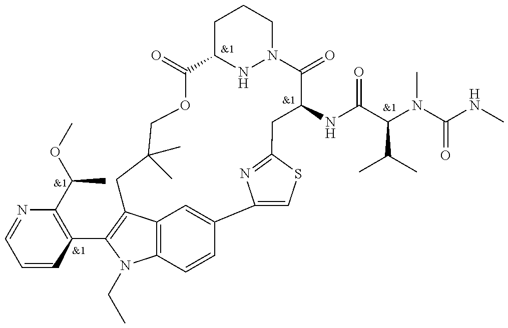 |
| AA138 | 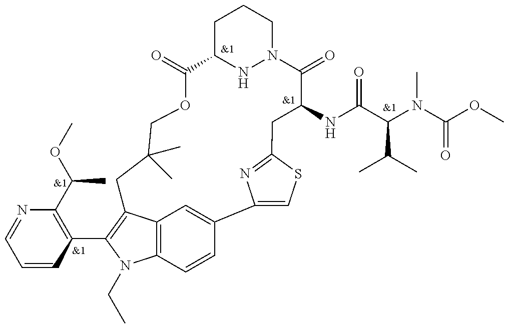 |
| AA139 | 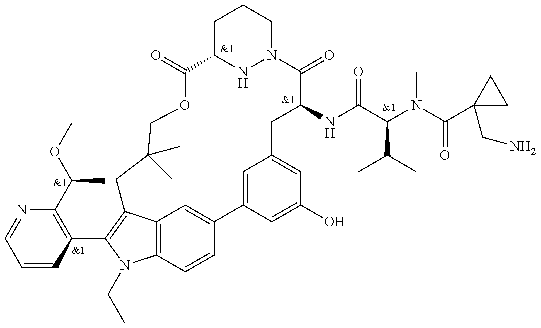 |
| AA140 | 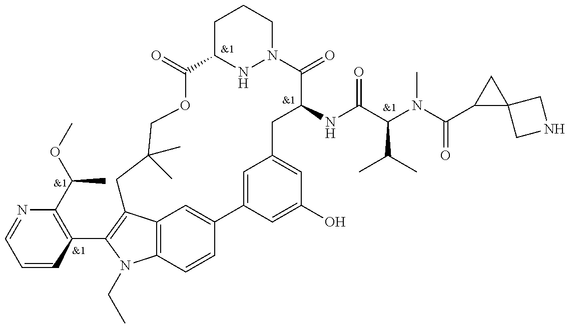 |
| AA141 | 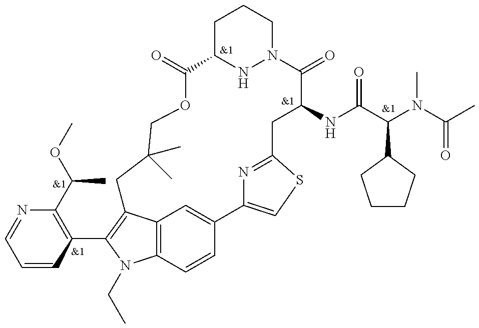 |
| AA142 | 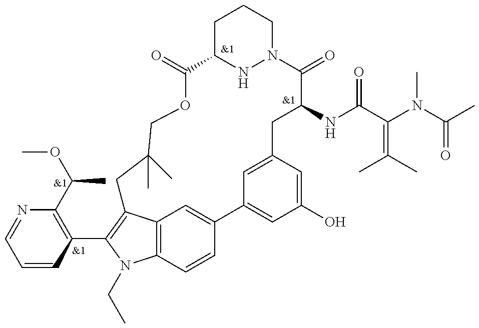 |
| AA143 | 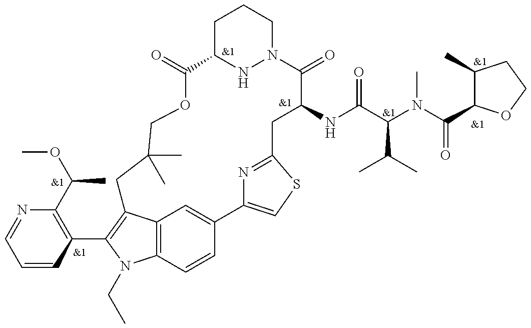 |
| AA144 | 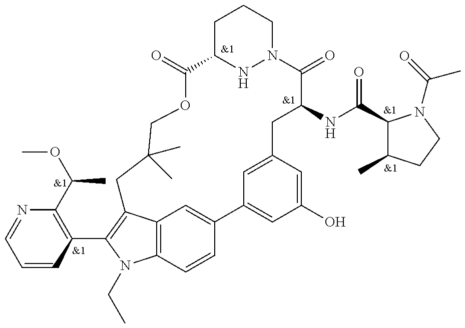 |
| AA145 | 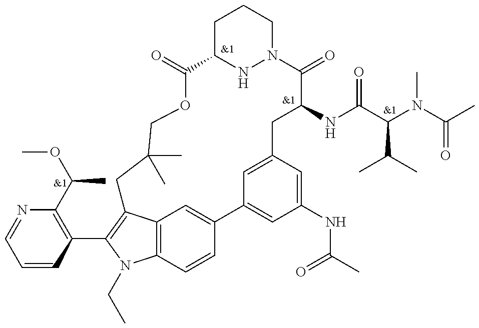 |
| AA146 | 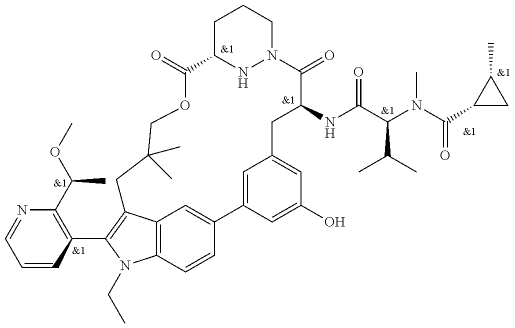 |
| AA147 | 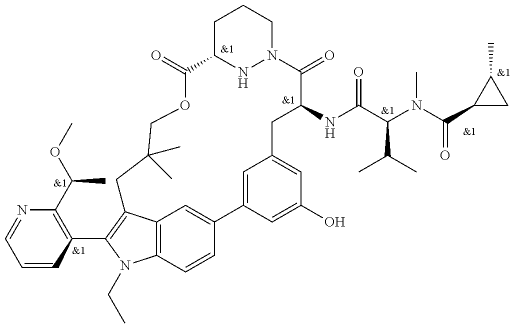 |
| AA148 | 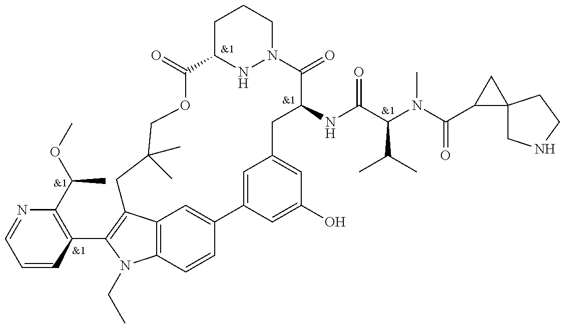 |
| AA149 | 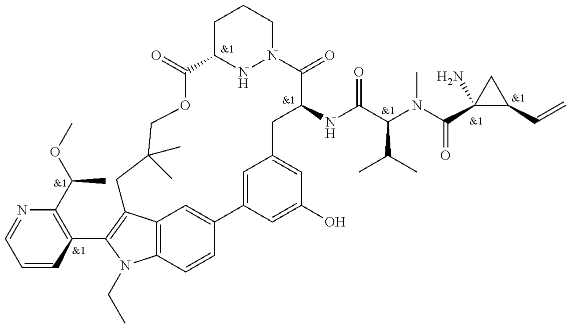 |
| AA150 | 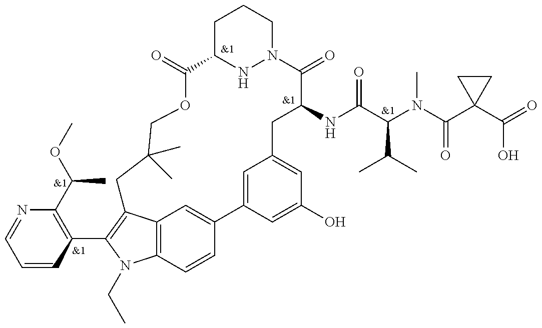 |
| AA151 | 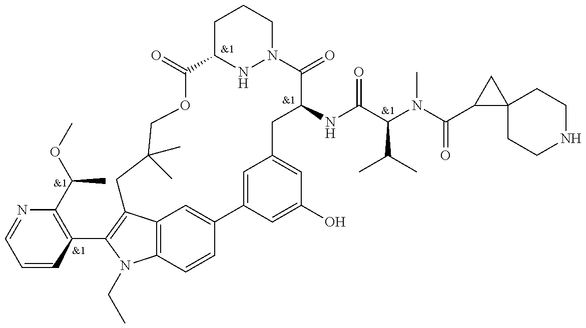 |
| AA152 | 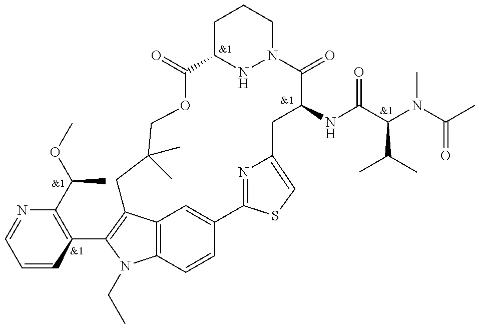 |
| AA153 | 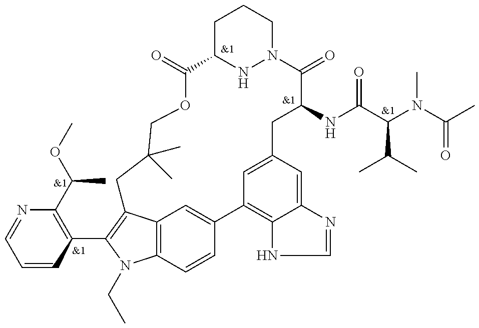 |
| AA154 | 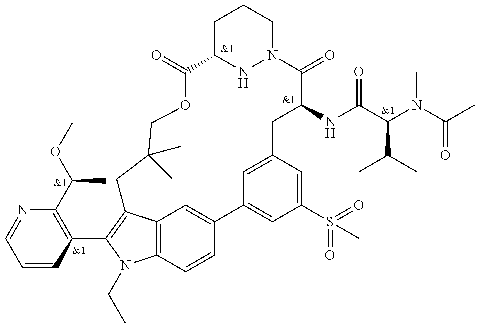 |
| AA155 | 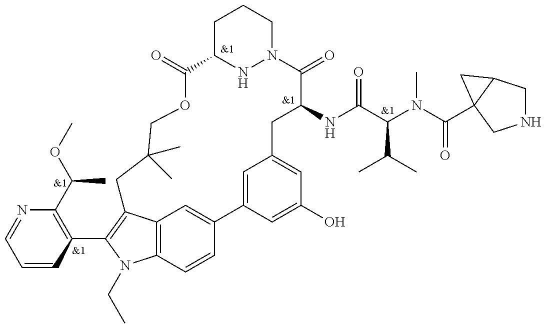 |
| AA156 | 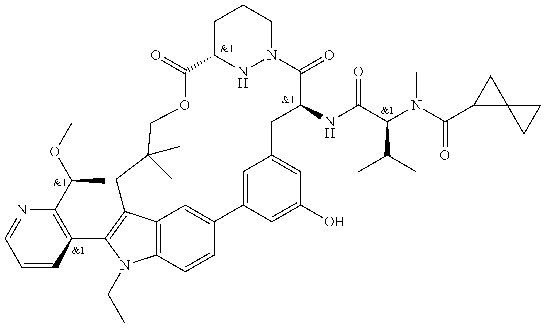 |
| AA157 | 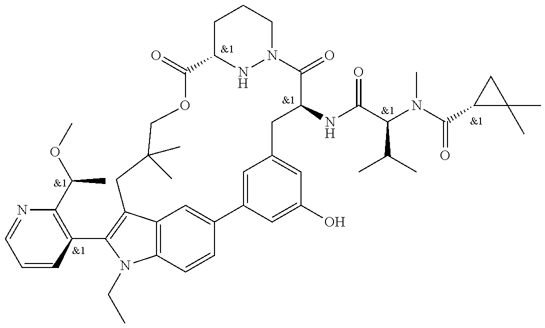 |
| AA158 | 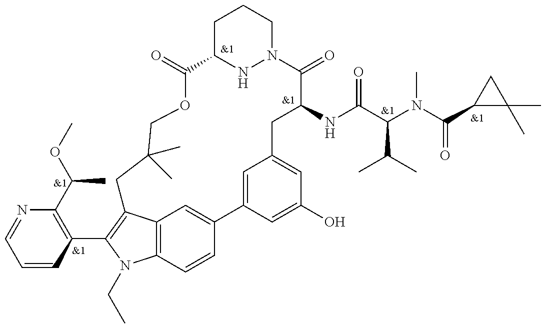 |
| AA159 | 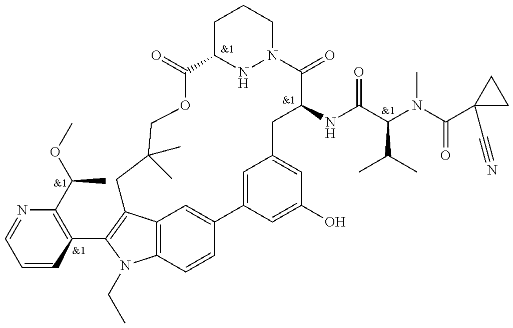 |
| AA160 | 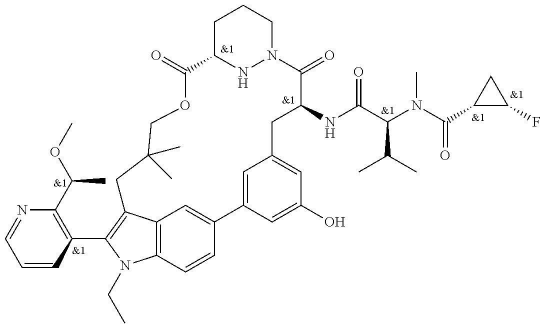 |
| AA161 | 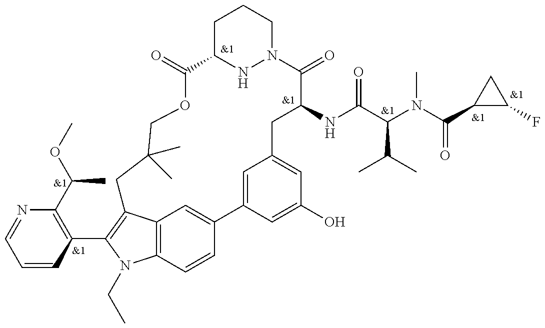 |
| AA162 | 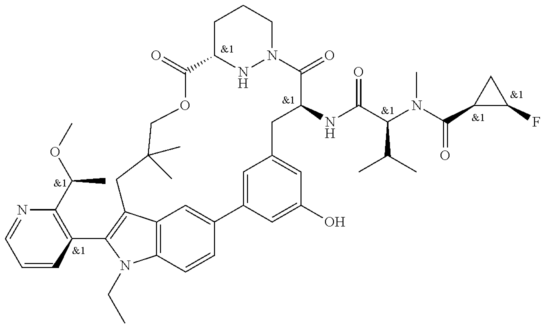 |
| AA163 | 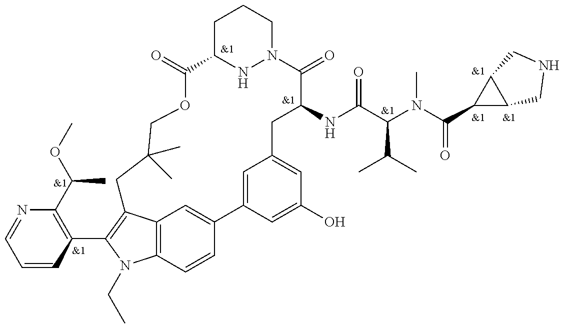 |
| AA164 | 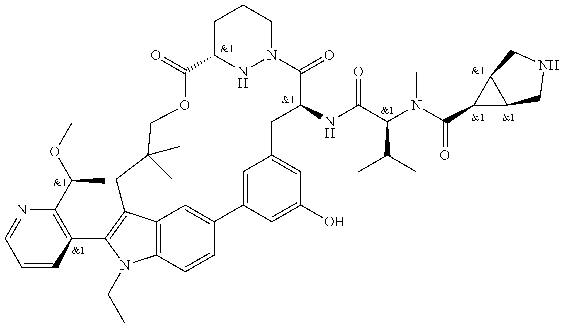 |
| AA165 | 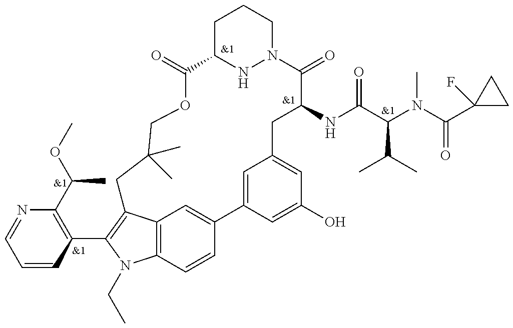 |
| AA166 | 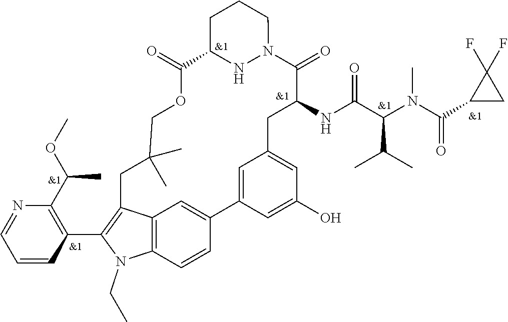 |
| AA167 | 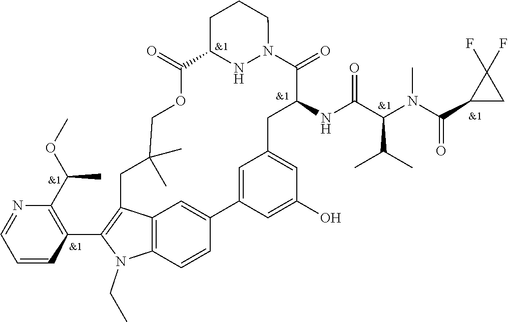 |
| AA168 | 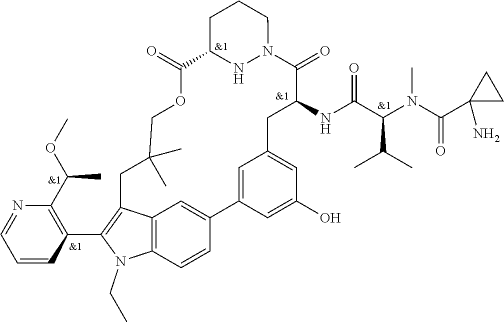 |
| AA169 | 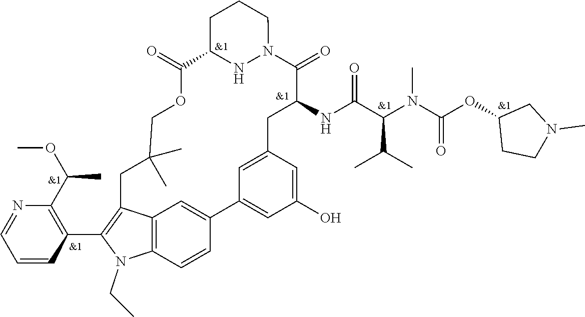 |
| AA170 | 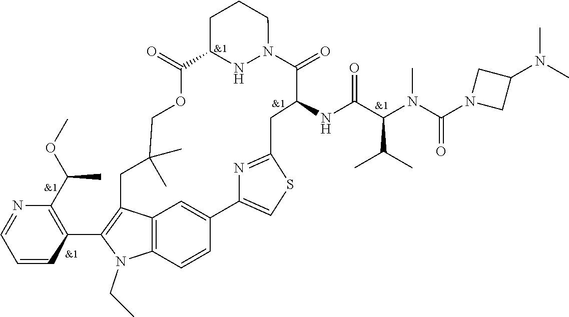 |
| AA171 | 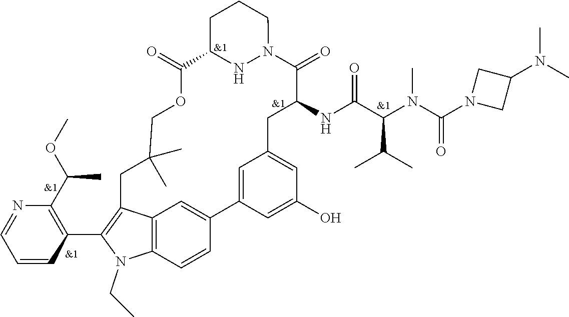 |
| AA172 | 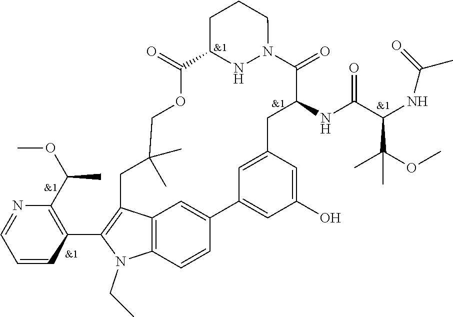 |
| AA173 | 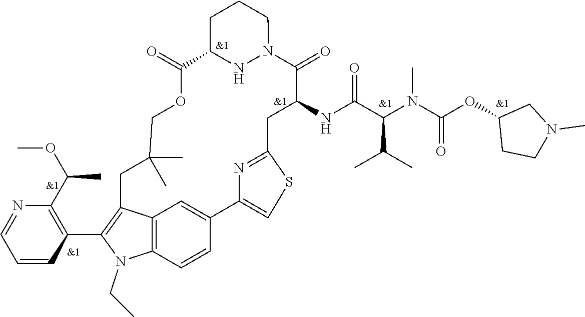 |
| AA174 | 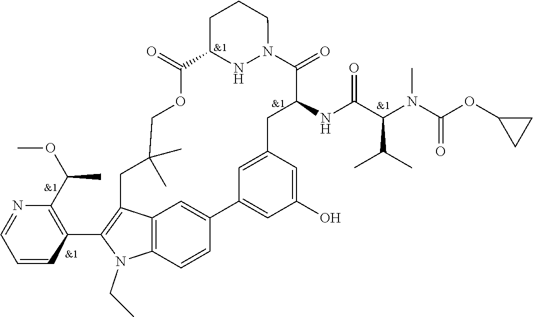 |
| AA175 | 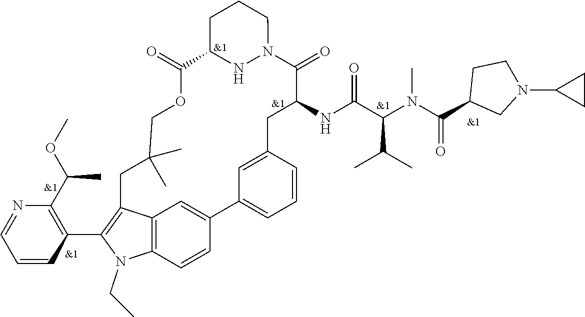 |
| AA176 | 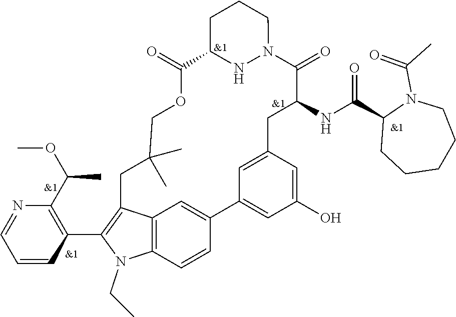 |
| AA177 | 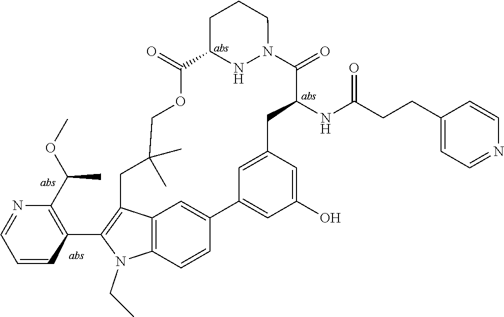 |
| AA178 | 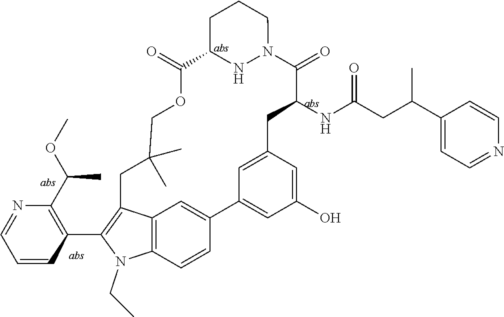 |
| AA179 | 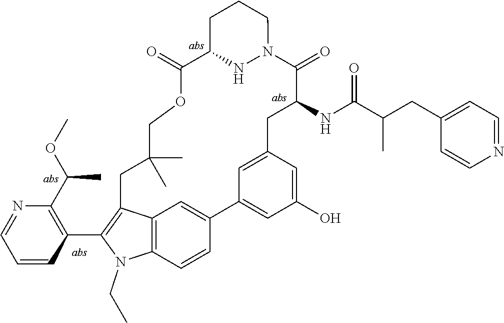 |
| AA180 | 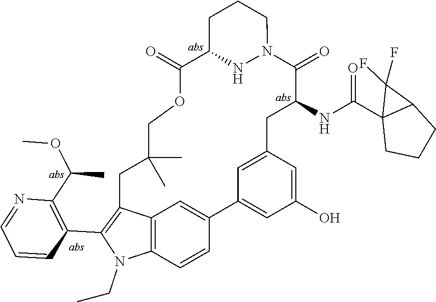 |
| AA181 | 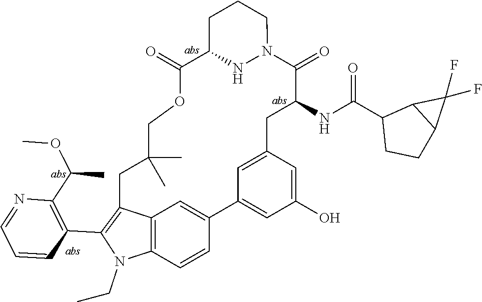 |
| AA182 | 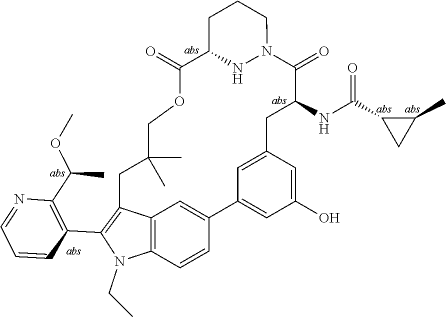 |
| AA183 | 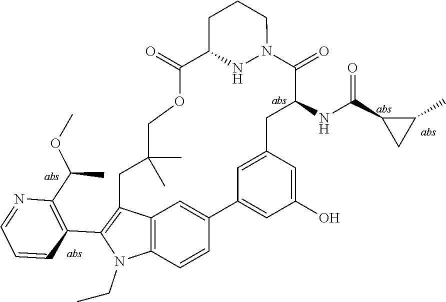 |
| AA184 | 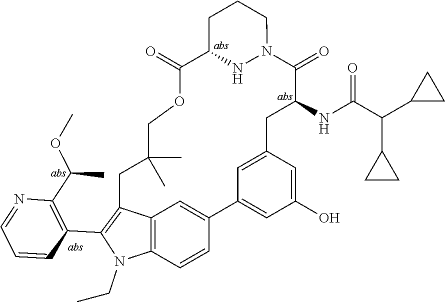 |
| AA185 | 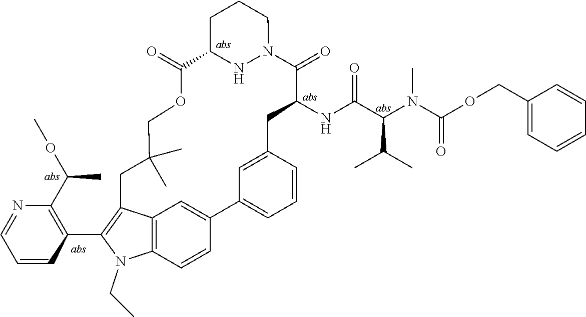 |
| AA186 | 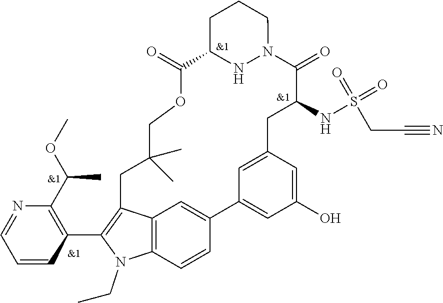 |
| AA187 | 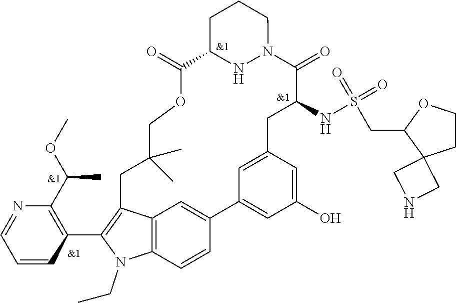 |
| AA188 | 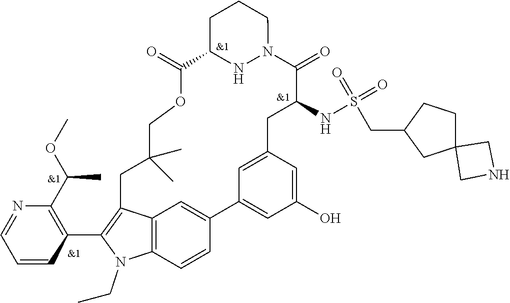 |
| AA189 | 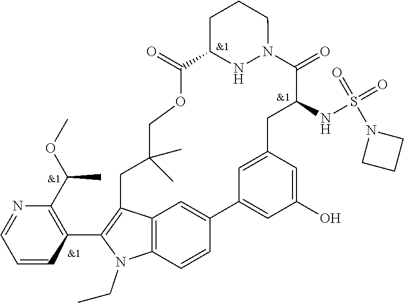 |
| AA190 | 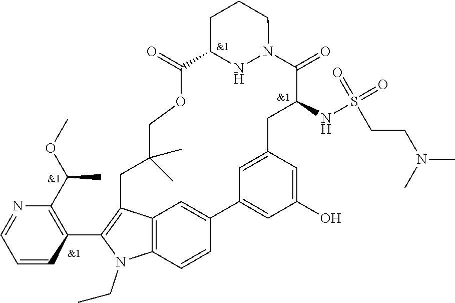 |
| AA191 | 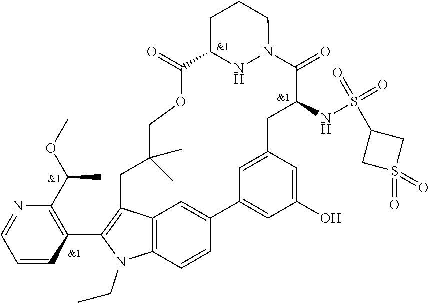 |
| AA192 | 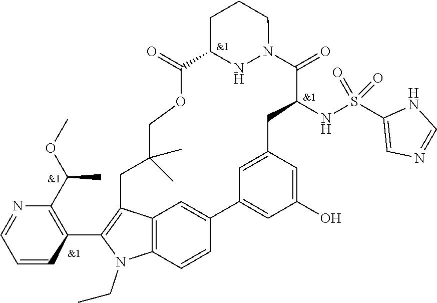 |
| AA193 | 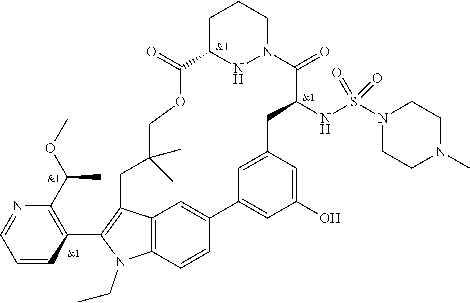 |
| AA194 | 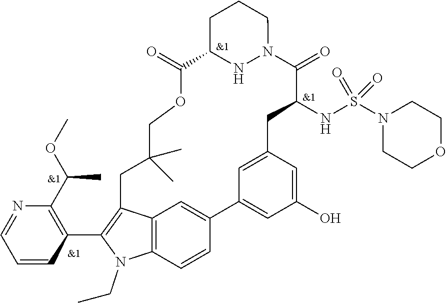 |
| AA195 | 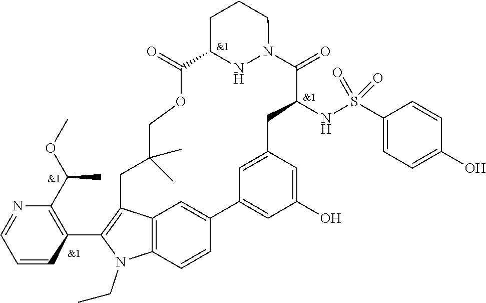 |
| AA196 | 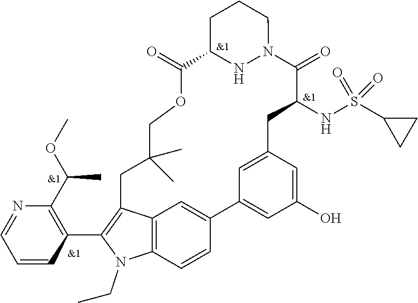 |
| AA197 | 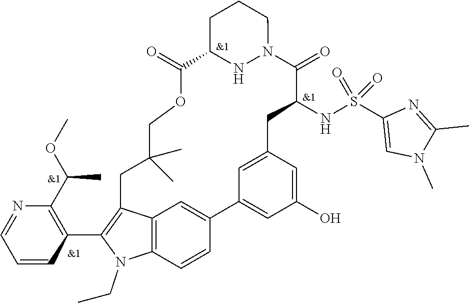 |
| AA198 | 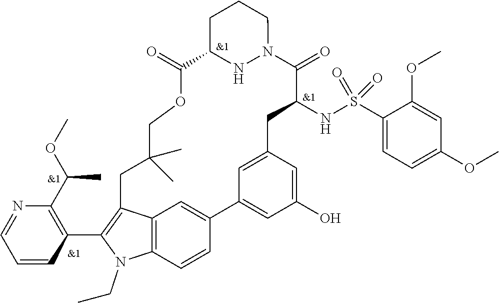 |
| AA199 | 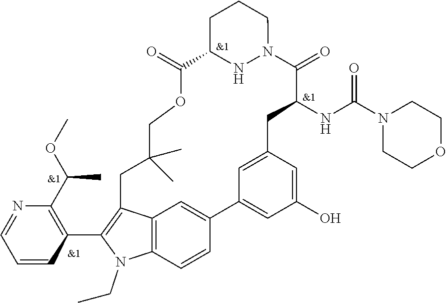 |
| AA200 | 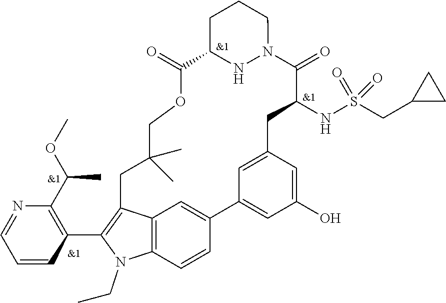 |
| AA201 | 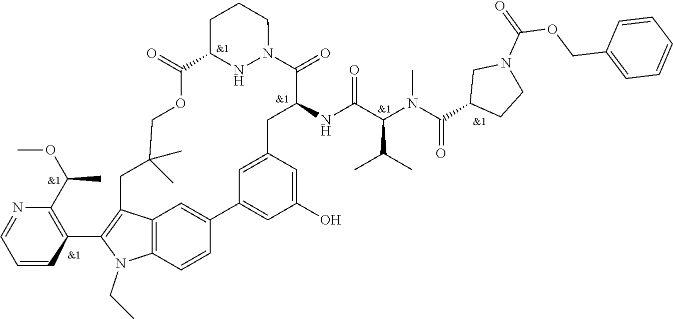 |
| AA202 | 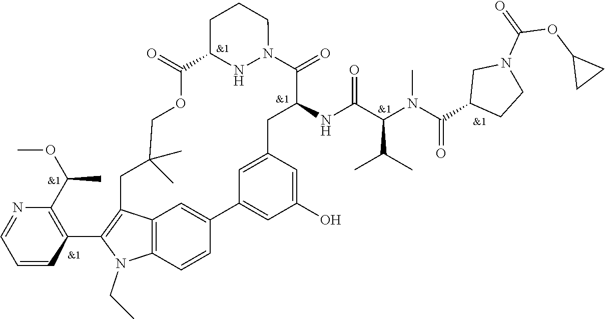 |
| AA203 | 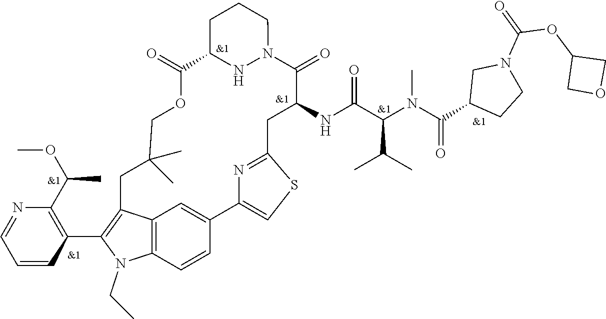 |
| AA204 | 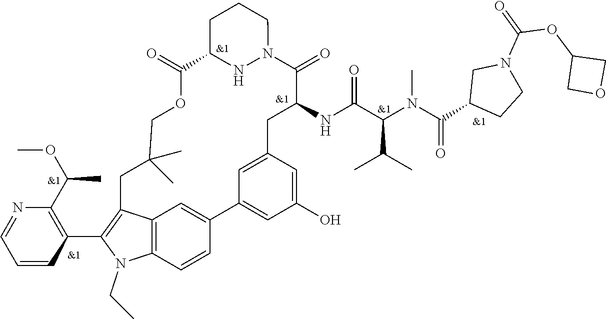 |
| AA205 | 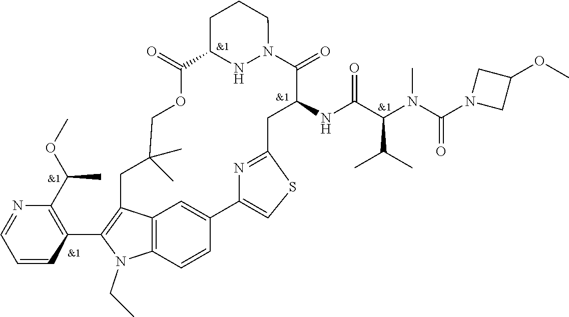 |
| AA206 | 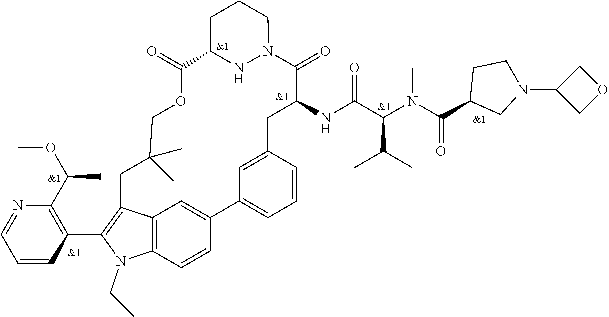 |
| AA207 | 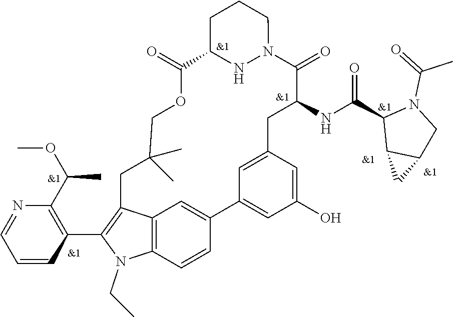 |
| AA208 | 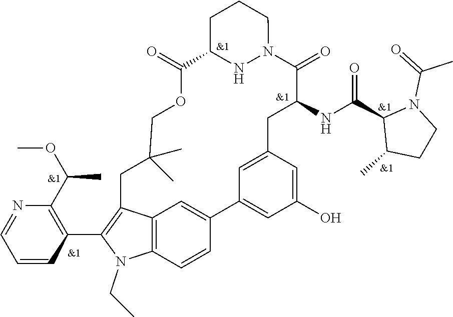 |
| AA209 | 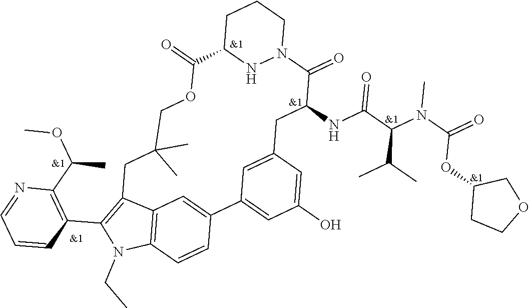 |
| AA210 | 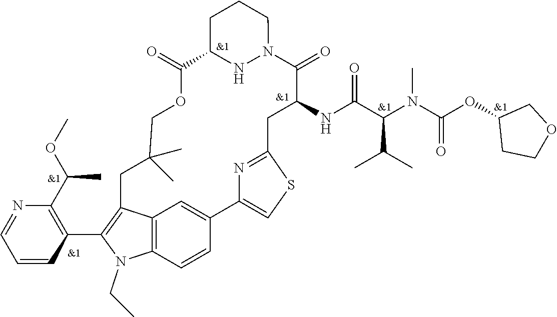 |
| AA211 | 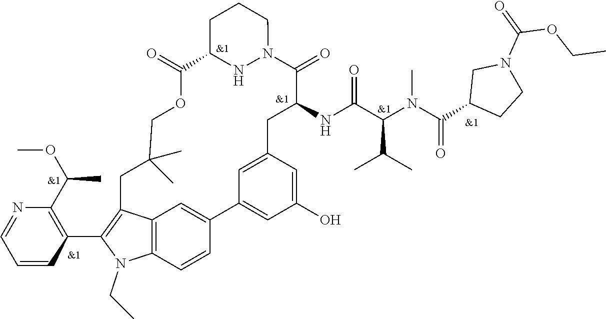 |
| AA212 | 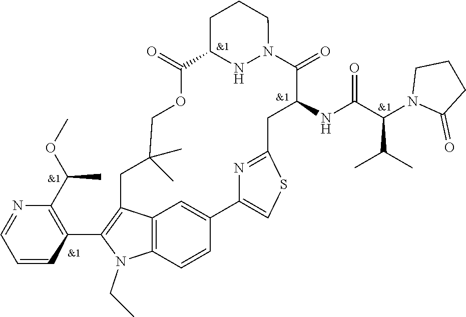 |
| AA213 | 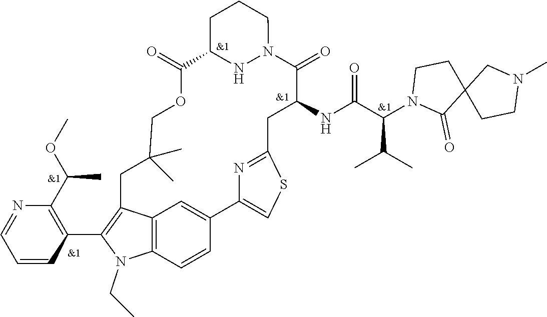 |
| AA214 | 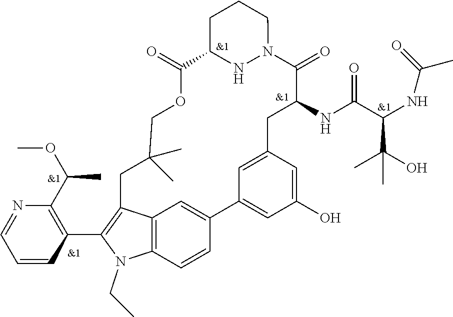 |
| AA215 | 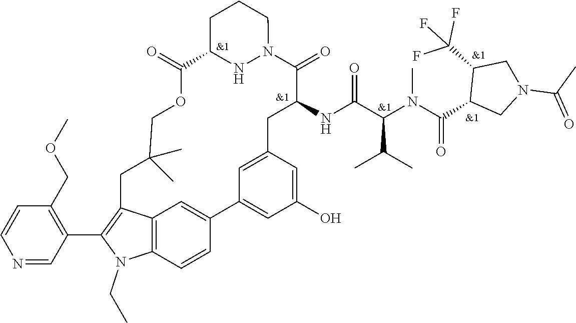 [SMILES: CCn1c(-c2cnccc2COC)c2c3cc(ccc31)-c1cc(O)cc(c1)C[C@H](NC(=O)[C@H](C(C)C)N(C)C(=O)[C@H]1CN(C(C)=O)C[C@H]1C(F)(F)F)C(=O)N1CCC[C@H](N1)C(=O)OCC(C)(C)C2] |
| AA216 | 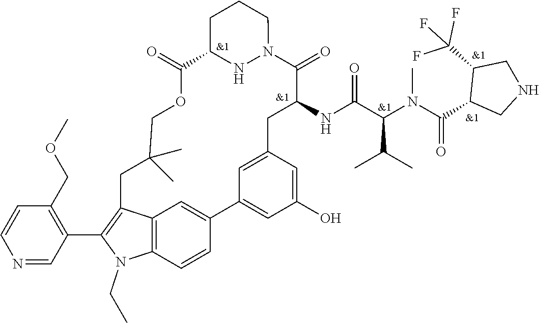 [SMILES: CCn1c(-c2cnccc2COC)c2c3cc(ccc31)-c1cc(O)cc(c1)C[C@H](NC(=O)[C@H](C(C)C)N(C)C(=O)[C@H]1CNC[C@H]1C(F)(F)F)C(=O)N1CCC[C@H](N1)C(=O)OCC(C)(C)C2] |
| AA217 | 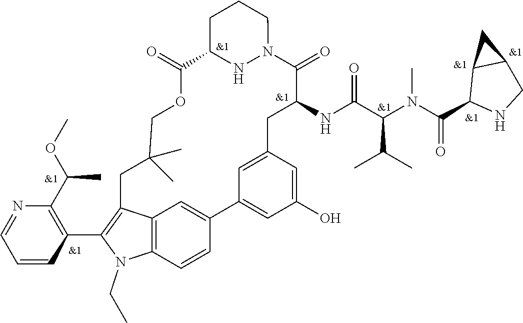 |
| AA218 | 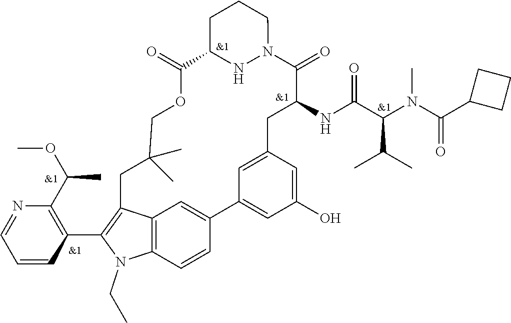 |
| AA219 | 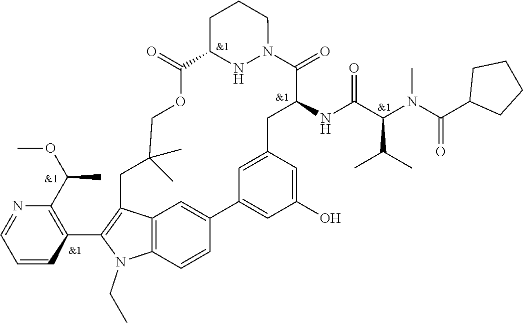 |
| AA220 | 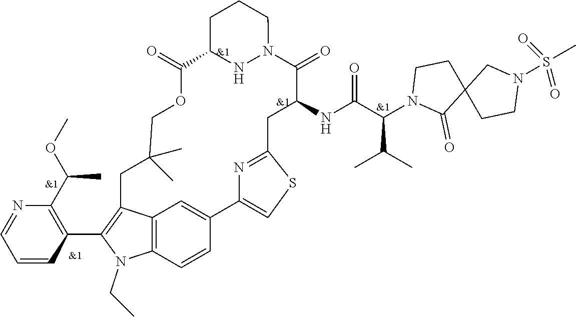 |
| AA221 | 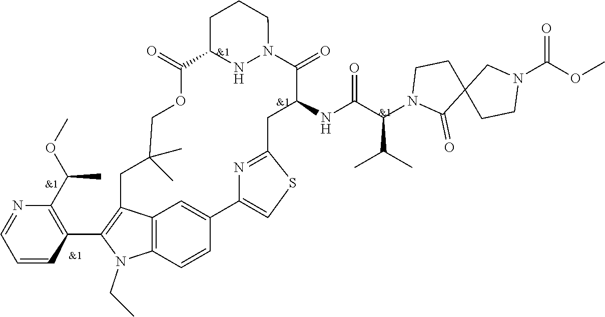 |
| AA222 | 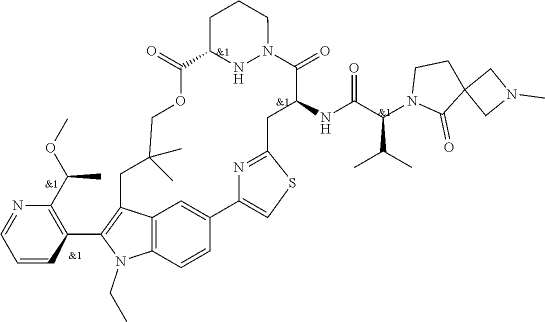 |
| AA223 | 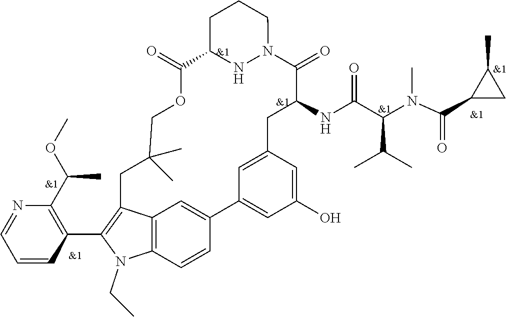 |
| AA224 | 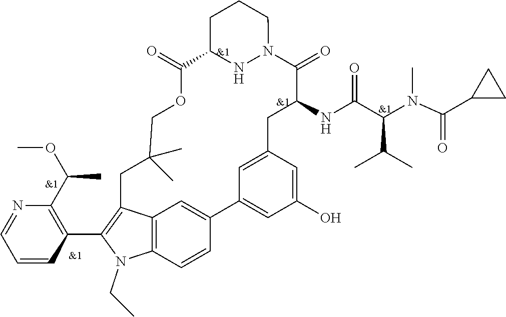 |
| AA225 | 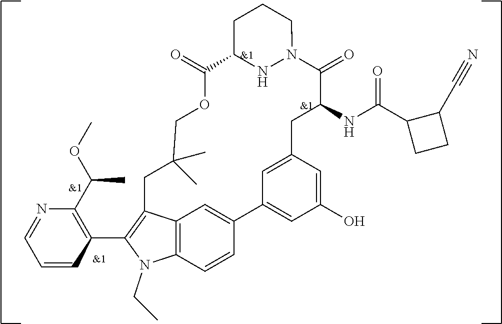 |
| AA226 | 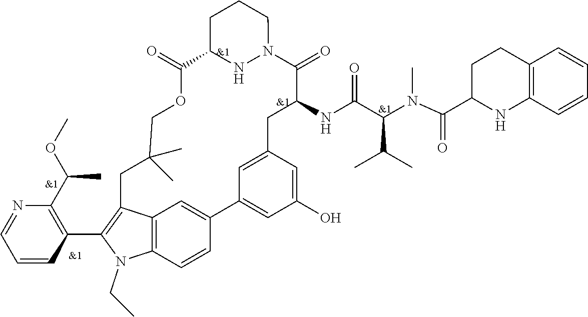 |
| AA227 | 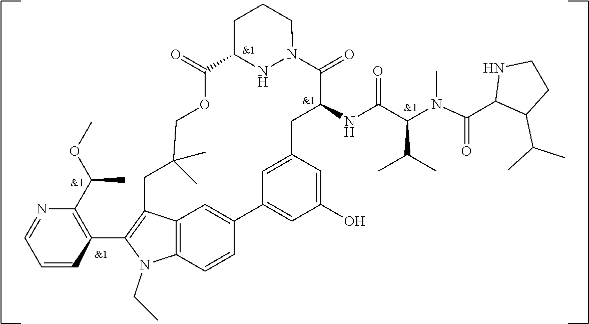 |
| AA228 | 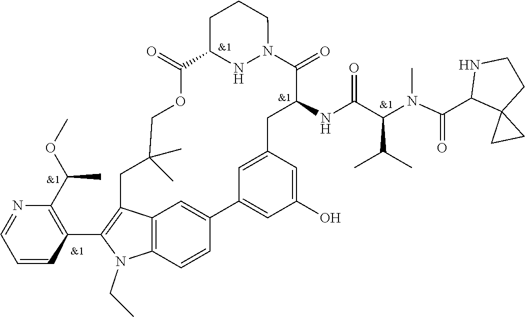 |
| AA229 | 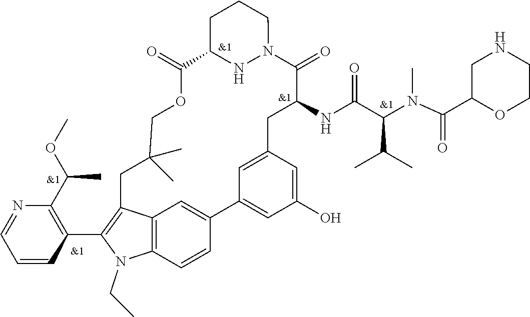 |
| AA230 | 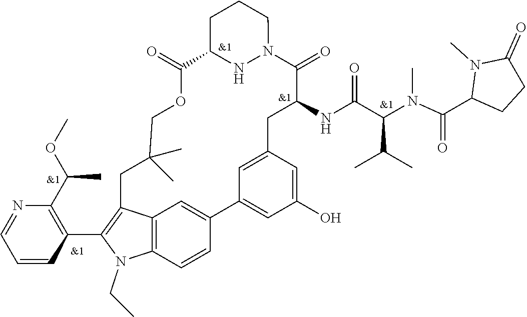 |
| AA231 | 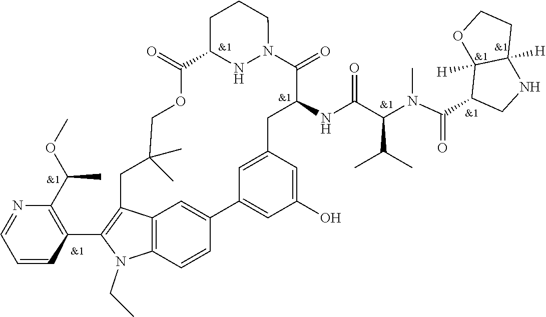 |
| AA232 | 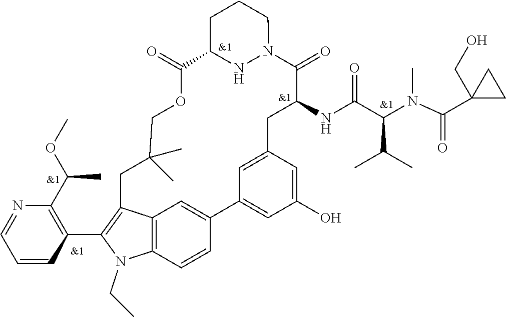 |
| AA233 | 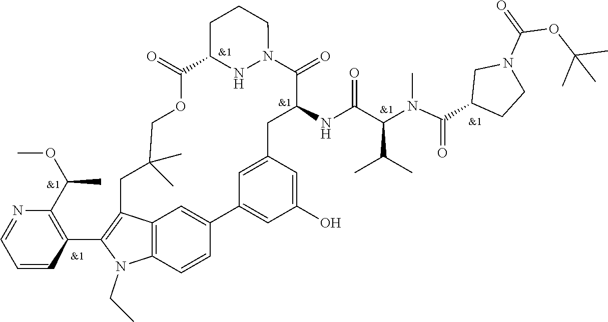 |
| AA234 | 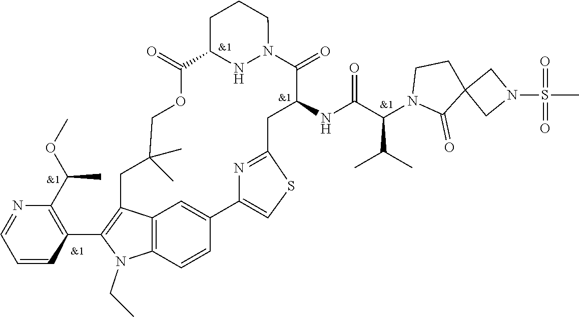 |
| AA235 | 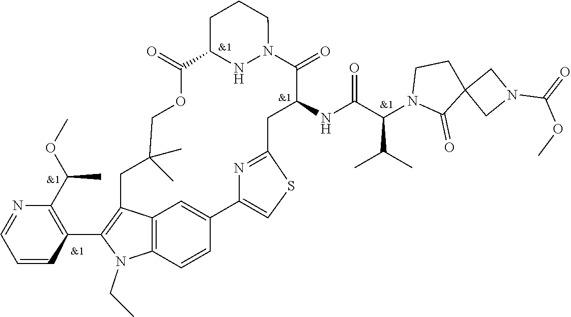 |
| AA236 | 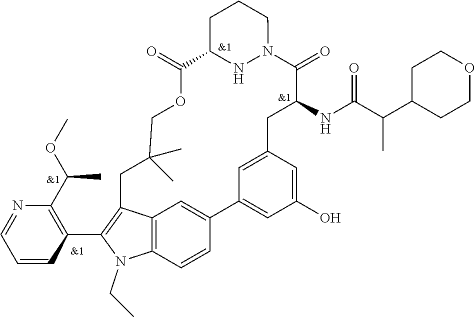 |
| AA237 | 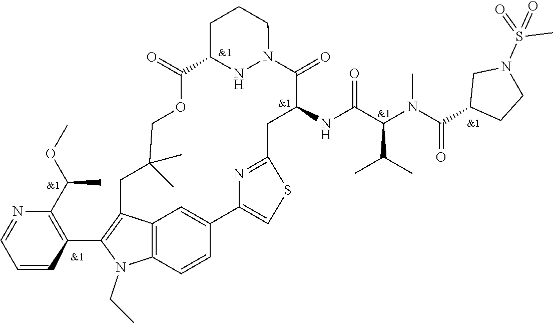 |
| AA238 | 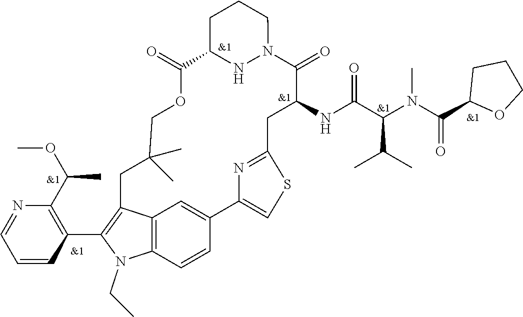 |
| AA239 | 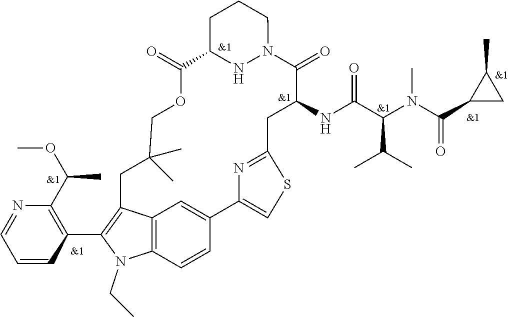 |
| AA240 | 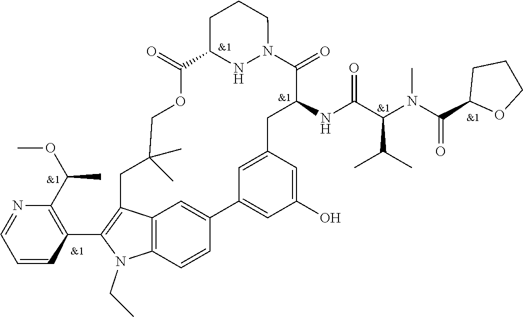 |
| AA241 | 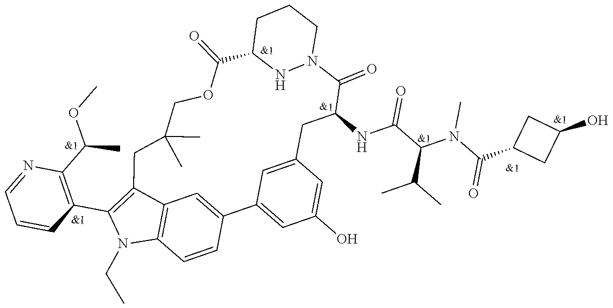 |
| AA242 | 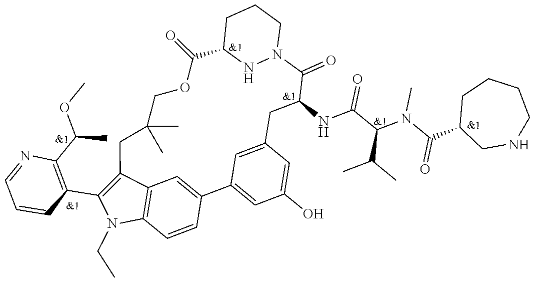 |
| AA243 | 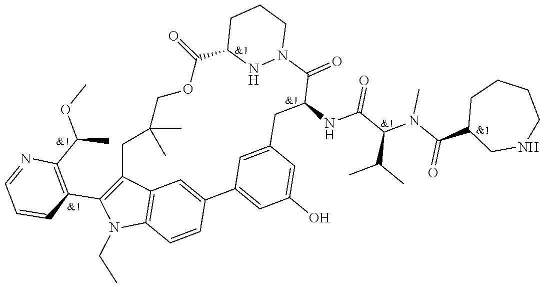 |
| AA244 | 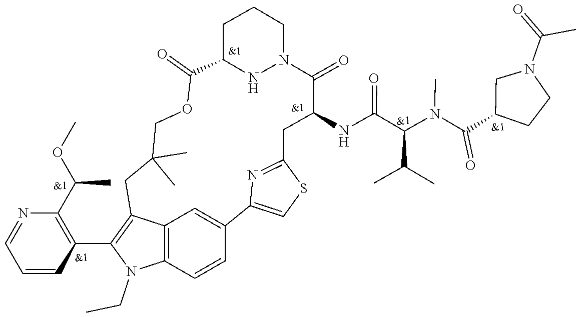 |
| AA245 | 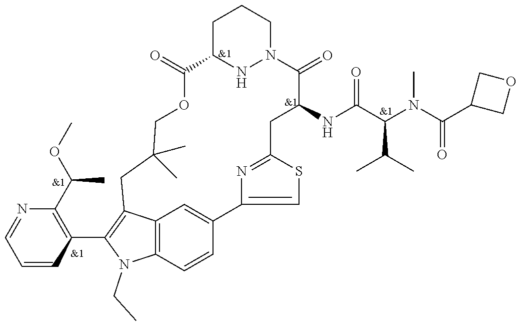 |
| AA246 | 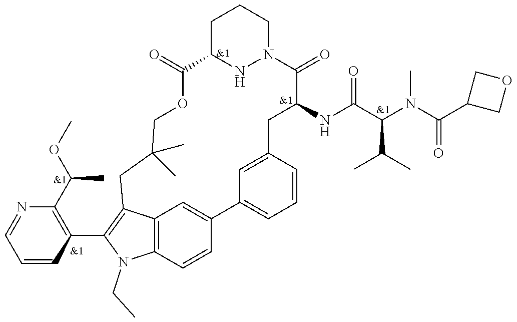 |
| AA247 | 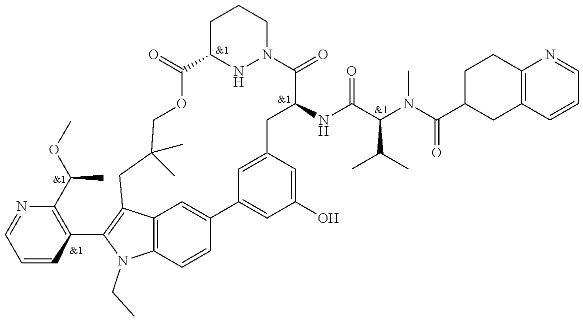 |
| AA248 | 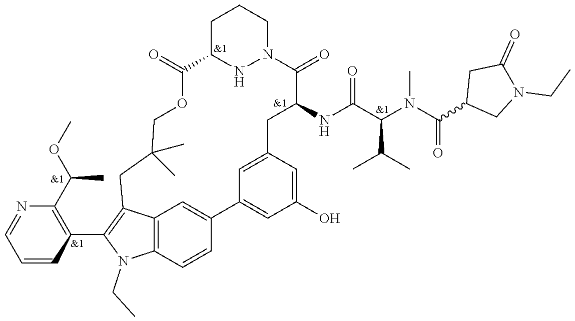 |
| AA249 | 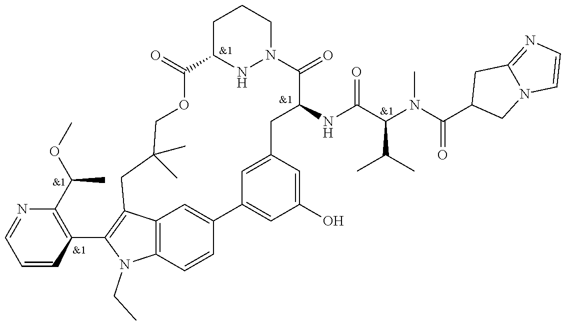 |
| AA250 | 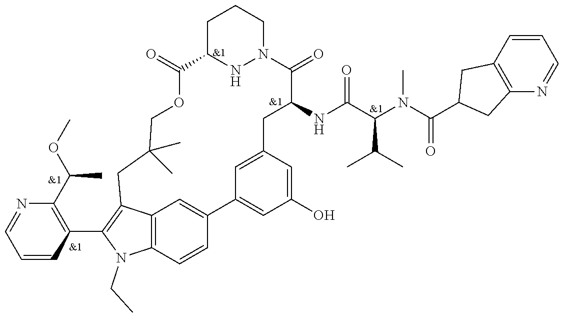 |
| AA251 | 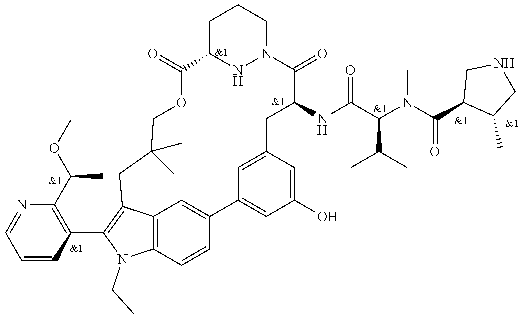 |
| AA252 | 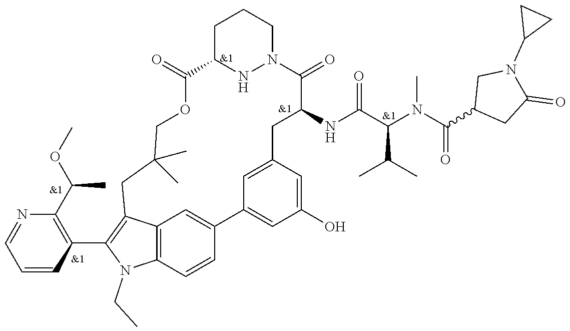 |
| AA253 | 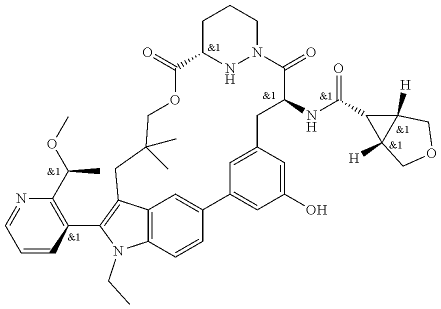 |
| AA254 | 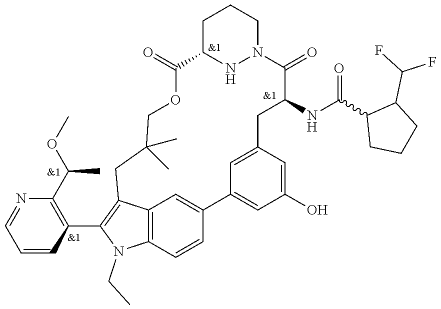 |
| AA255 | 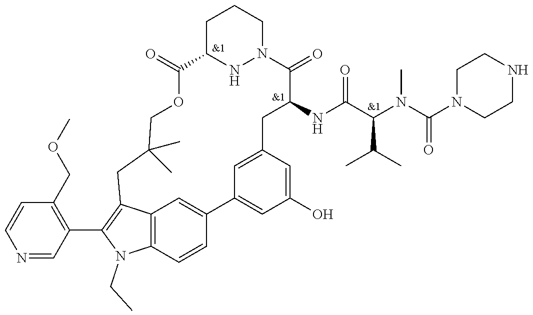 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AA256 | 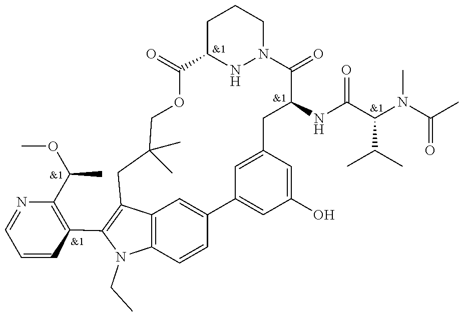 |
| AA257 | 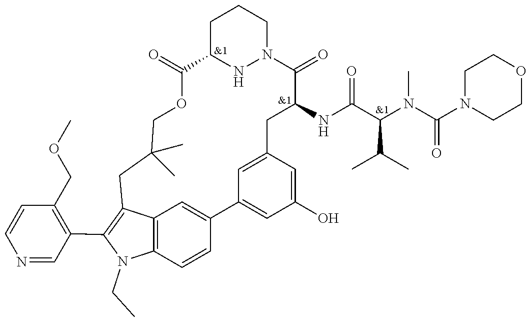 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AA258 | 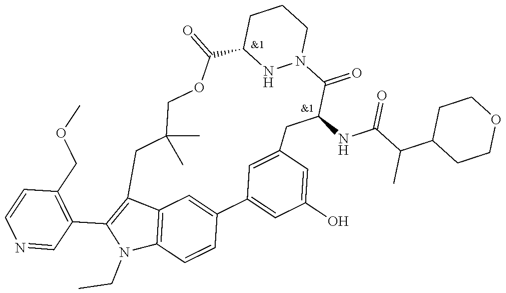 [SMILES: CCn1c(-c2cnccc2COC)c2c3cc(ccc31)-c1cc(O)cc(c1)C[C@H](NC(=O)C(C)C1CCOCC1)C(=O)N1CCC[C@H](N1)C(=O)OCC(C)(C)C2] |
| AA259 | 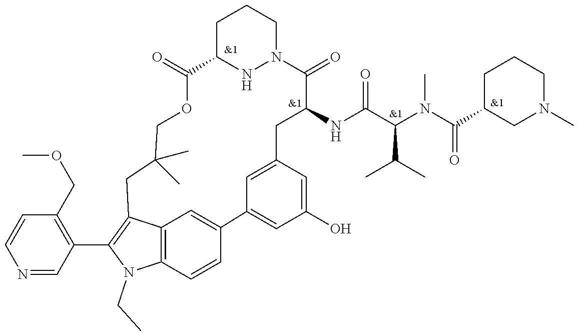 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| AA260 | 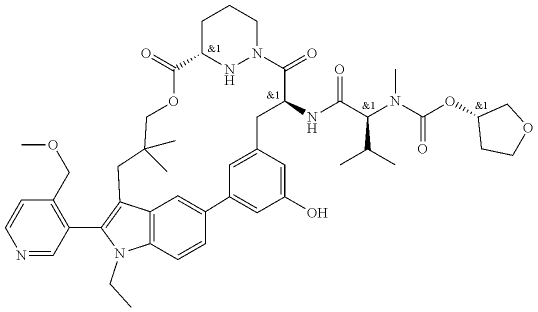 [SMILES: CCn1c(-c2cnccc2COC)c2c3cc(ccc31)-c1cc(O)cc(c1)C[C@H](NC(=O)[C@H](C(C)C)N(C)C(=O)O[C@H]1CCOC1)C(=O)N1CCC[C@H](N1)C(=O)OCC(C)(C)C2] |
| AA261 | 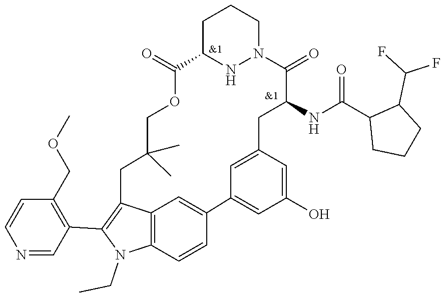 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| AA262 | 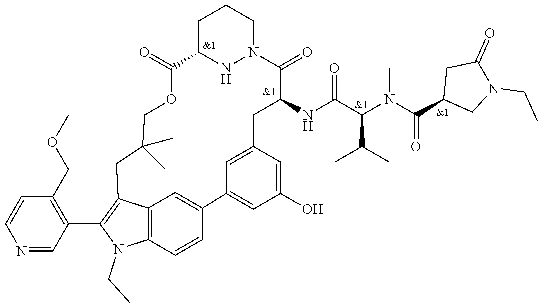 [SMILES: CCN1C[C@@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)CC1=O] |
| AA263 | 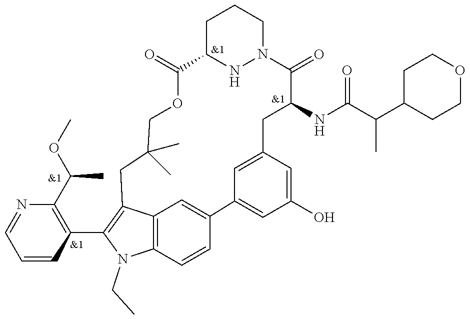 |
| AA264 | 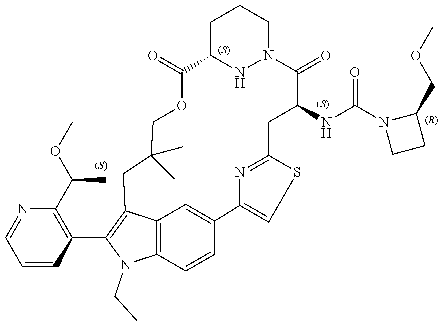 |
| AA265 | 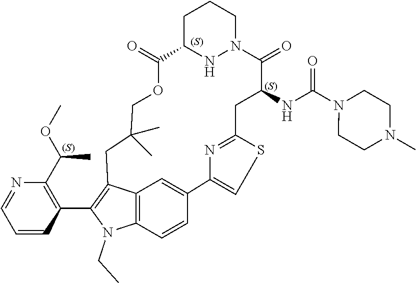 |
| AA266 | 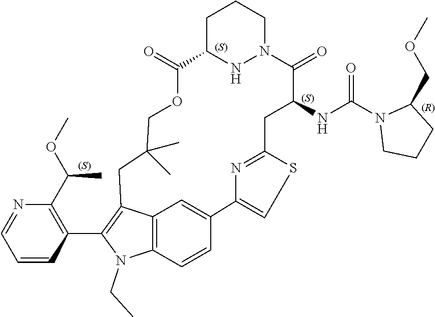 |
| AA267 | 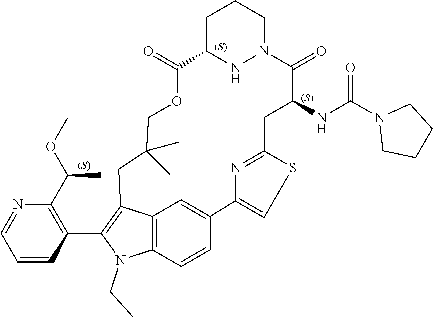 |
| AA268 | 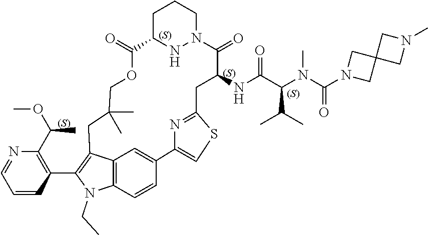 |
| AA270 | 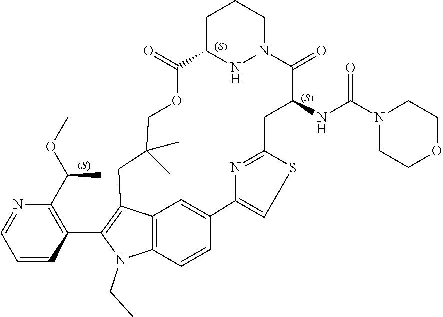 |
| AA271 | 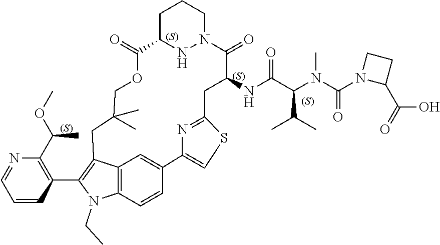 |
| AA272 | 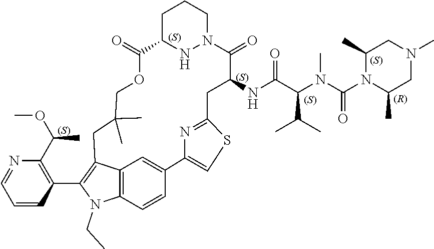 |
| AA273 | 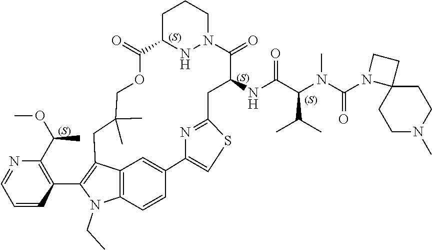 |
| AA274 | 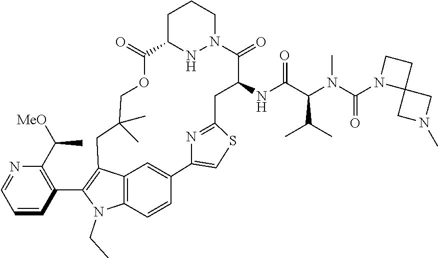 |
| AA275 | 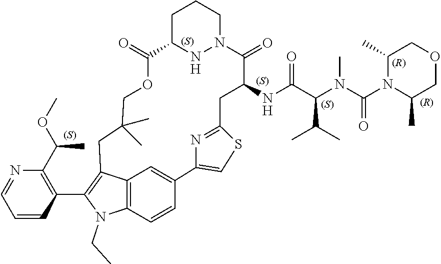 |
| AA276 | 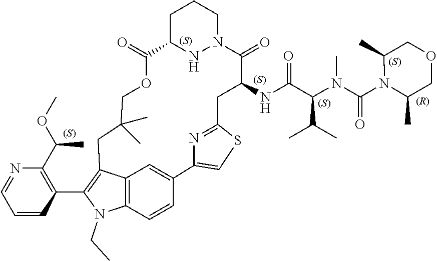 |
| AA277 | 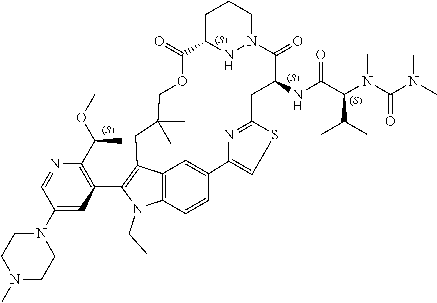 |
| AA278 | 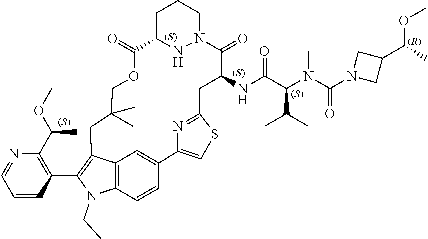 |
| AA279 | 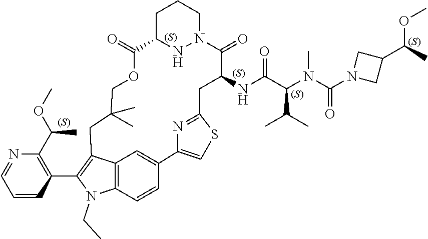 |
| AA280 | 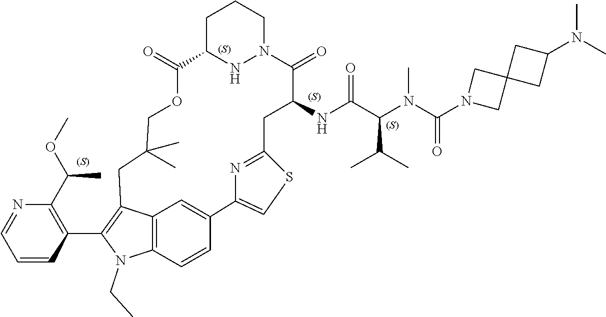 |
| AA281 | 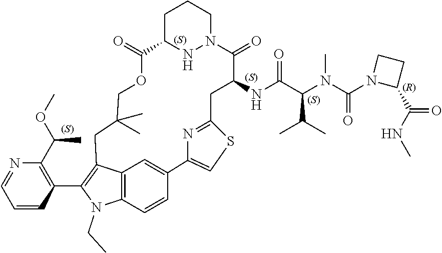 |
| AA282 | 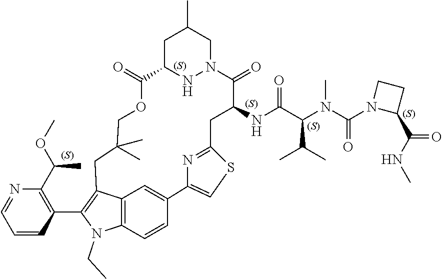 |
| AA283 | 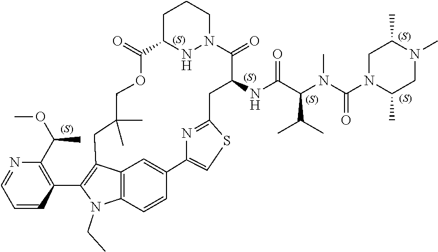 |
| AA284 | 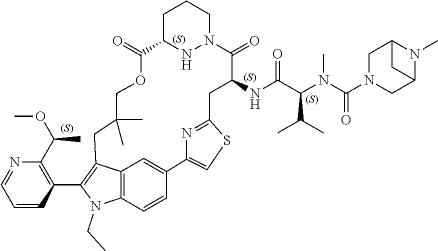 |
| AA285 | 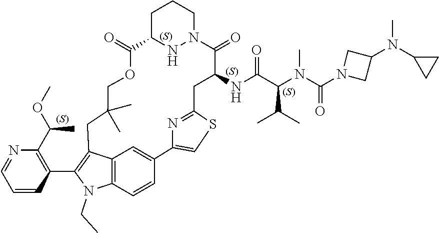 |
| AA286 | 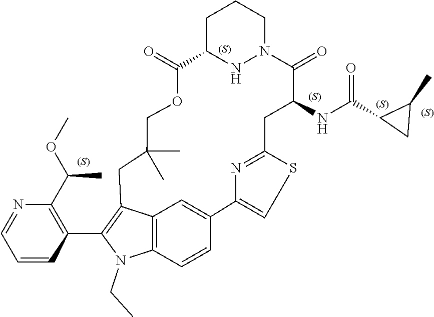 |
| AA287 | 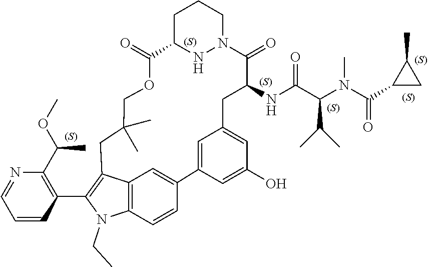 |
| AA288 | 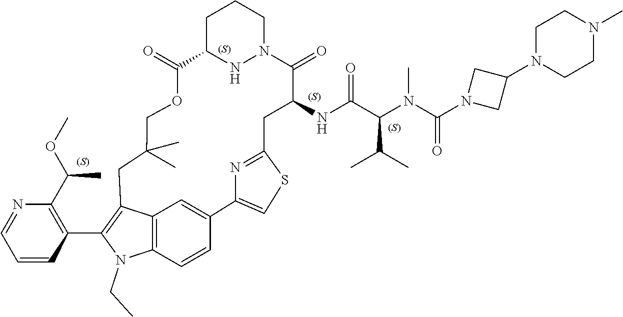 |
| AA289 | 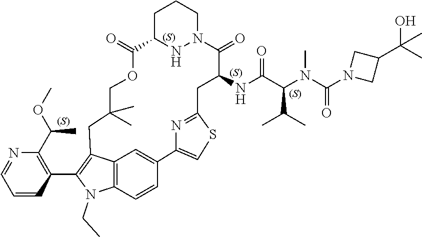 |
| AA290 | 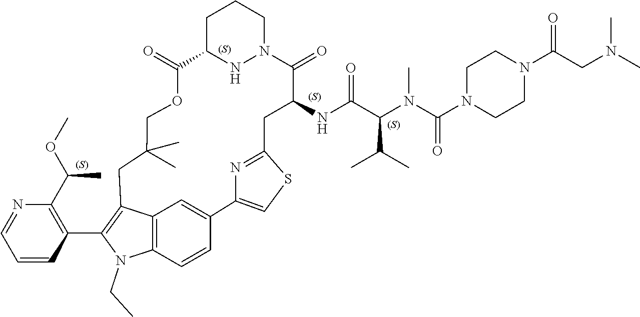 |
| AA291 | 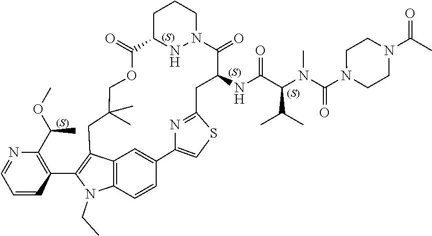 |
| AA292 | 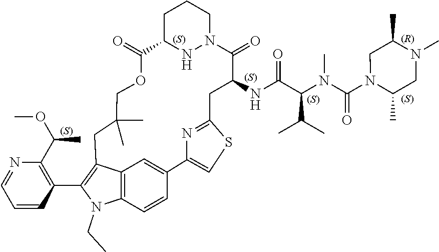 |
| AA293 | 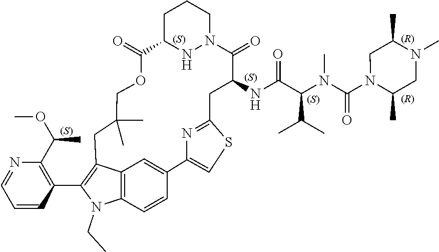 |
| AA294 | 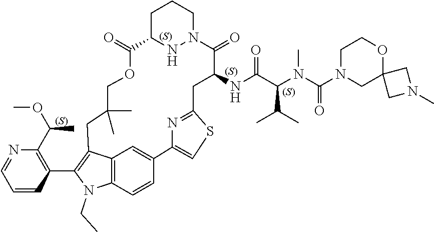 |
| AA295 | 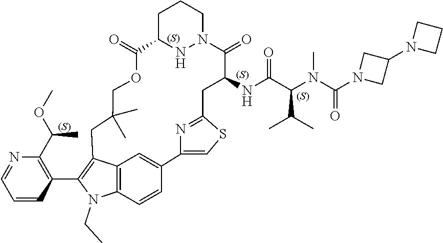 |
| AA296 | 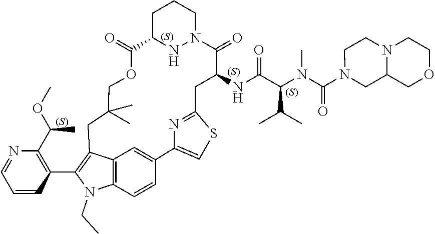 |
| AA297 | 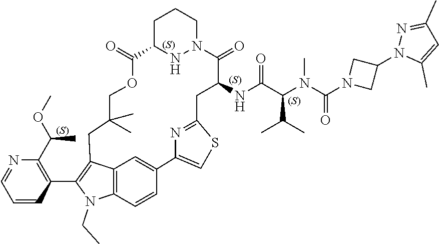 |
| AA298 | 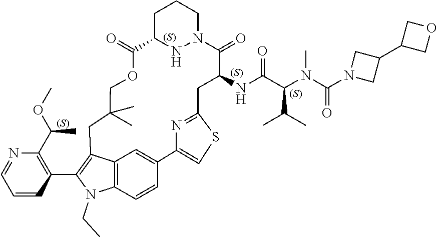 |
| AA299 | 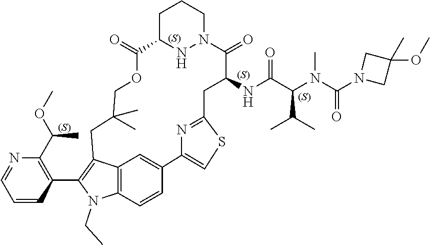 |
| AA300 | 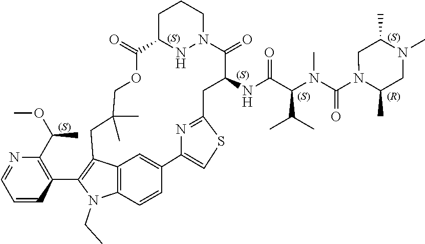 |
| AA301 | 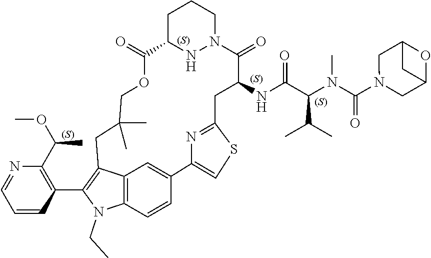 |
| AA302 | 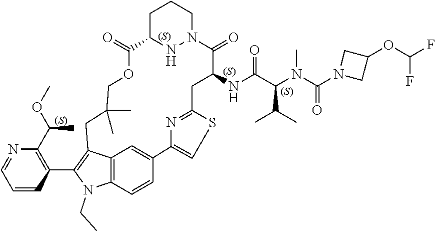 |
| AA303 | 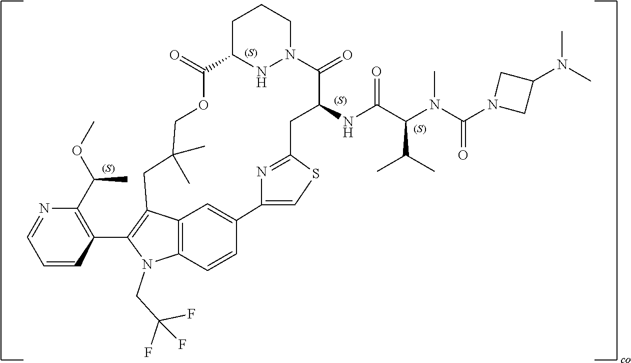 |
| AA304 | 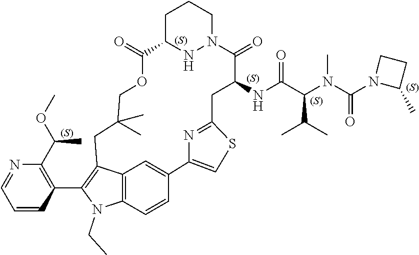 |
| AA305 | 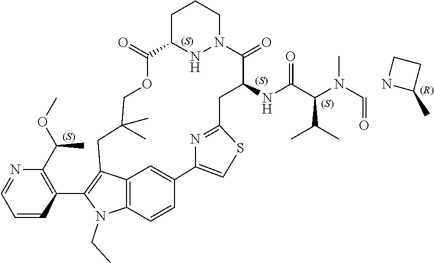 |
| AA306 | 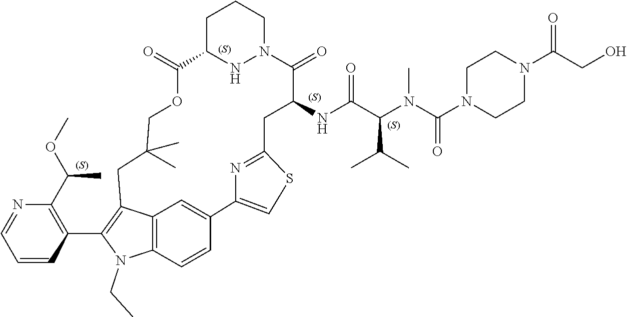 |
| AA307 | 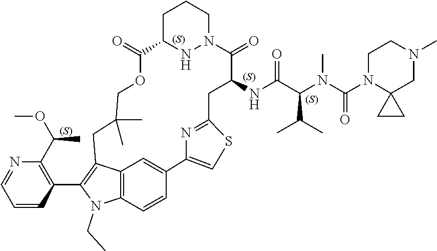 |
| AA308 | 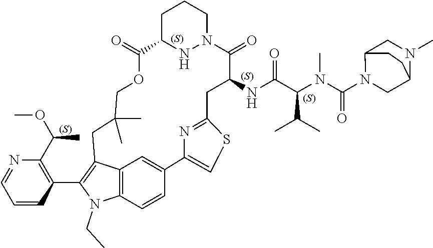 |
| AA309 | 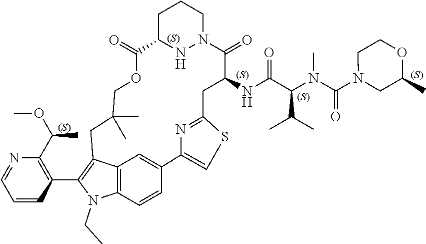 |
| AA310 | 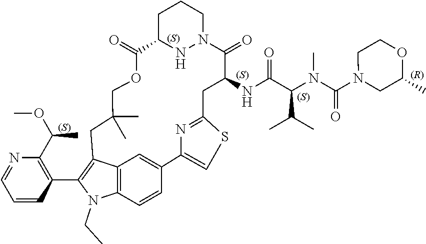 |
| AA311 | 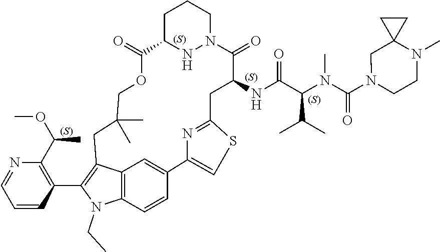 |
| AA312 | 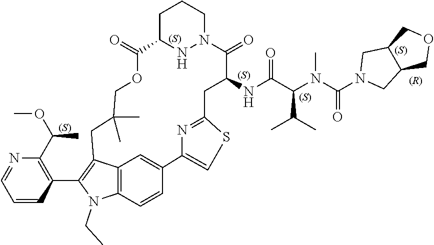 |
| AA313 | 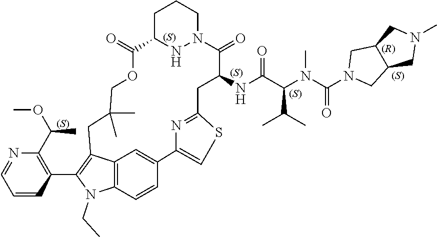 |
| AA314 | 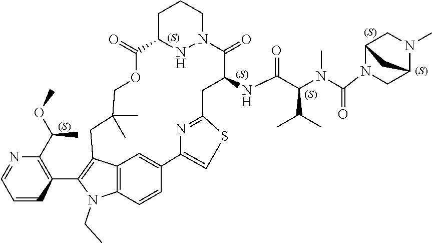 |
| AA315 | 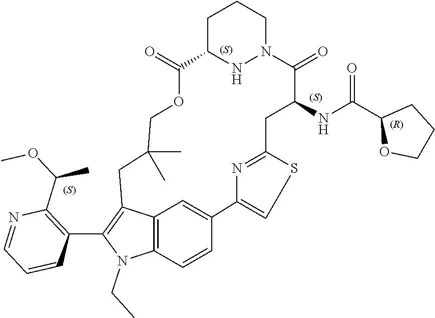 |
| AA316 | 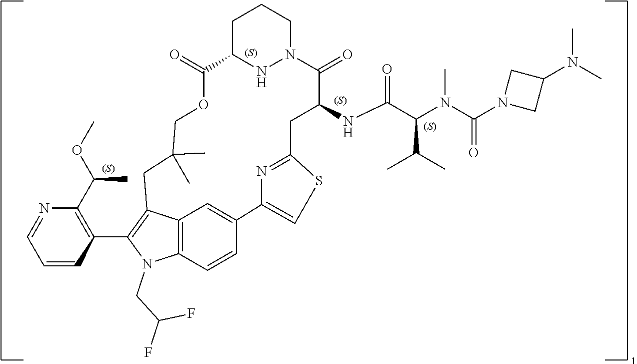 |
| AA317 | 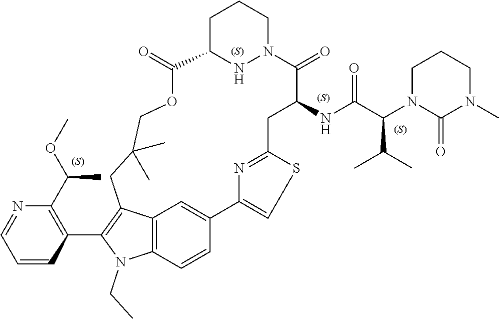 |
| AA318 | 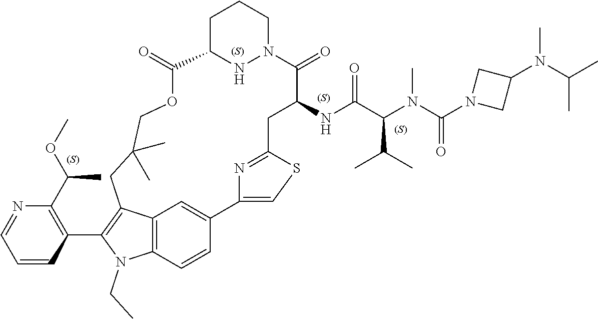 |
| AA319 | 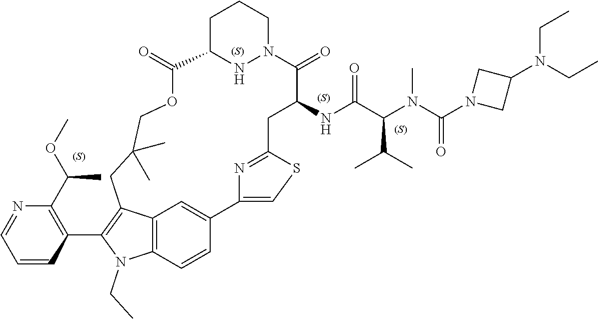 |
| AA320 | 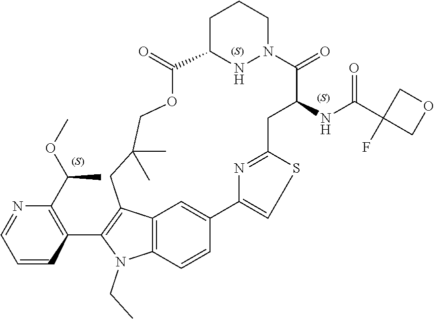 |
| AA321 | 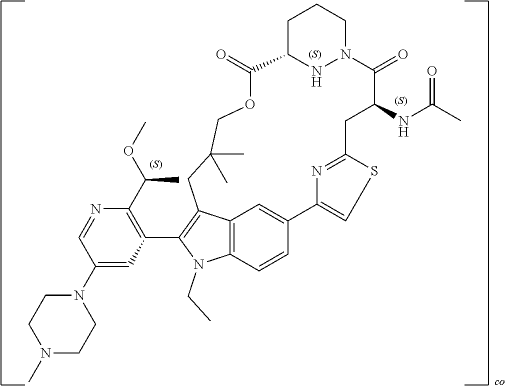 |
| AA322 | 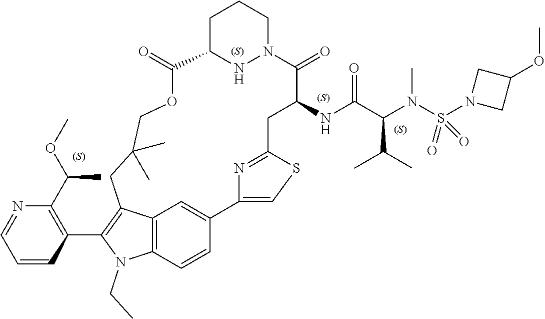 |
| AA323 | 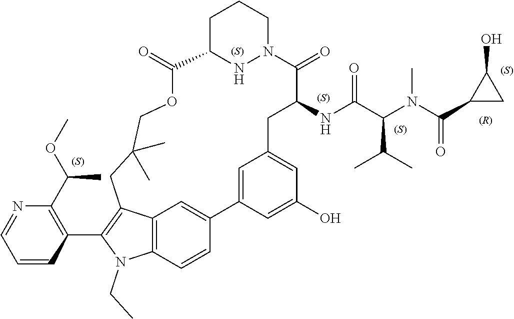 |
| AA324 | 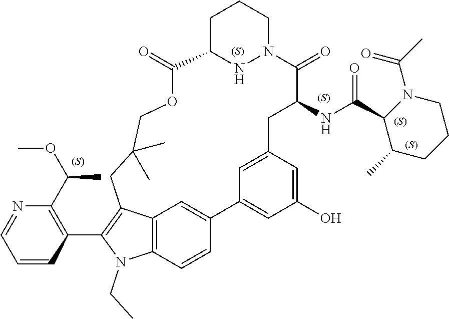 |
| AA325 | 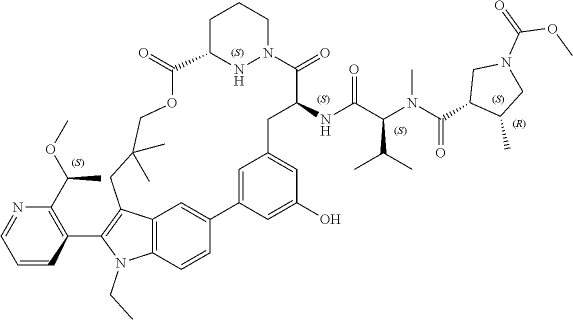 |
| AA326 | 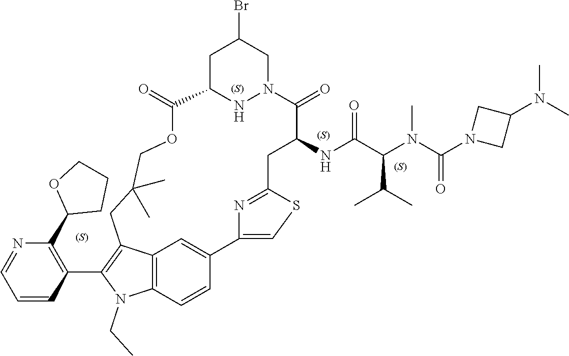 |
| AA327 | 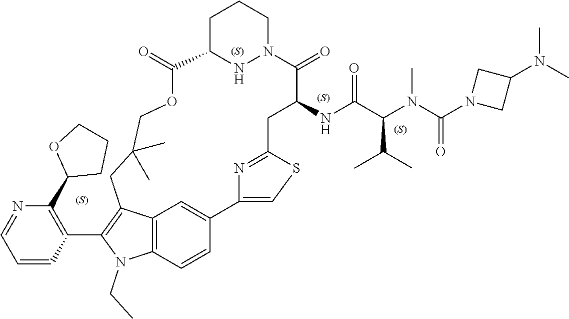 |
| AA328 | 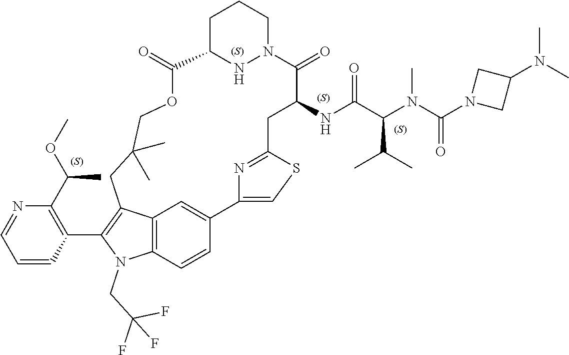 |
| AA329 | 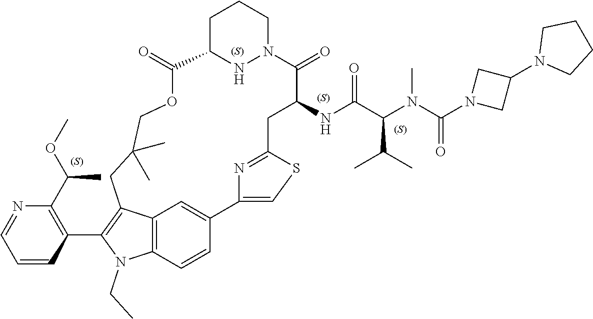 |
| AA330 | 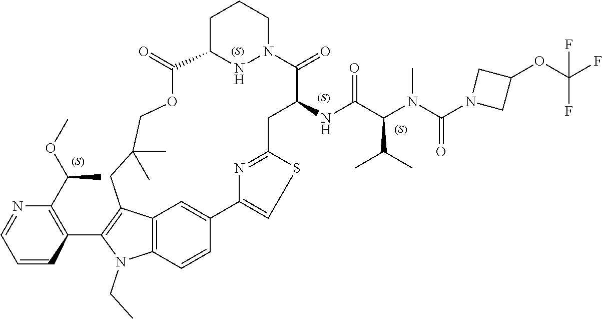 |
| AA331 | 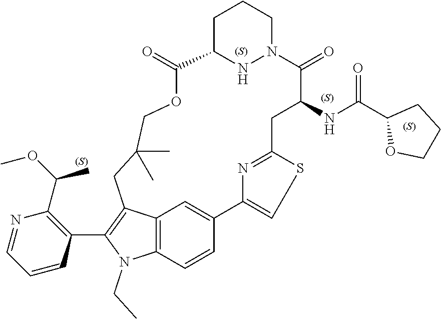 |
| AA332 | 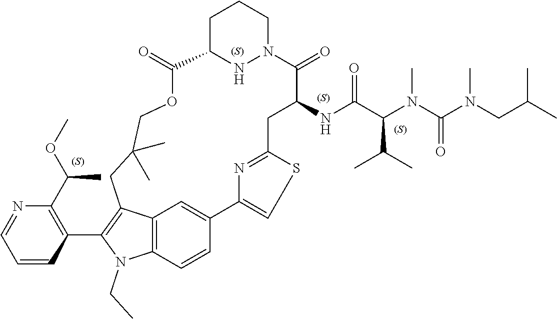 |
| AA333 | 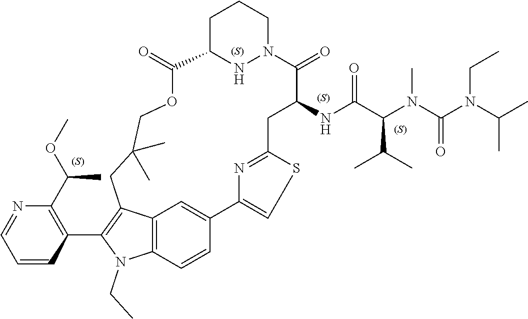 |
| AA334 | 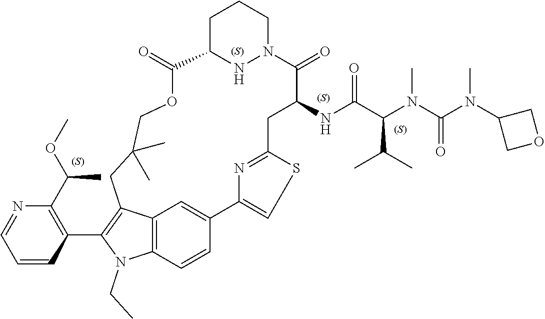 |
| AA335 | 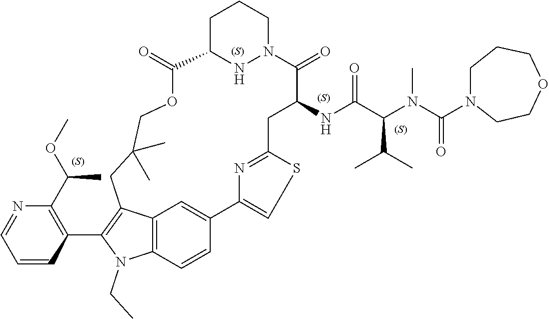 |
| AA336 | 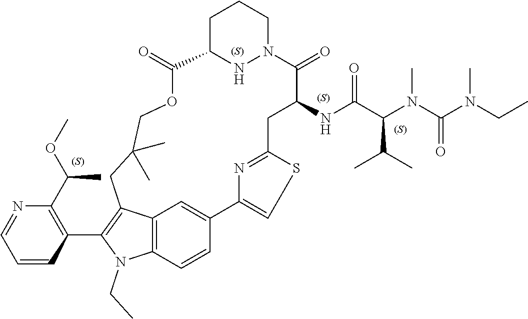 |
| AA337 | 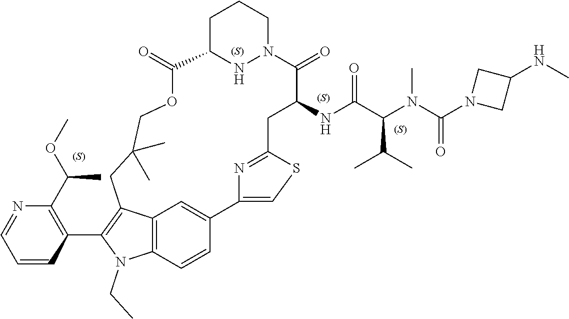 |
| AA338 | 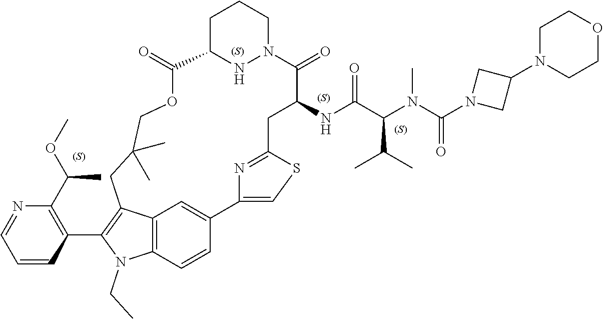 |
| AA339 | 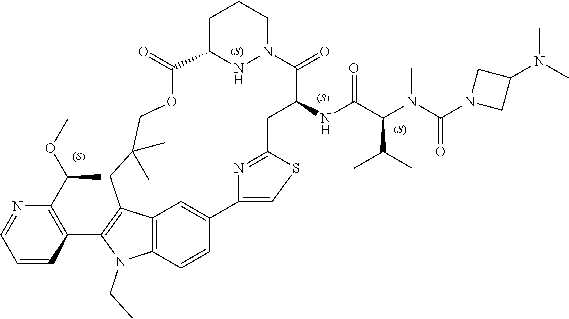 |
| AA340 | 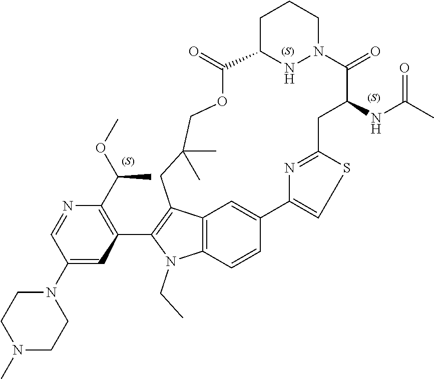 |
| AA341 | 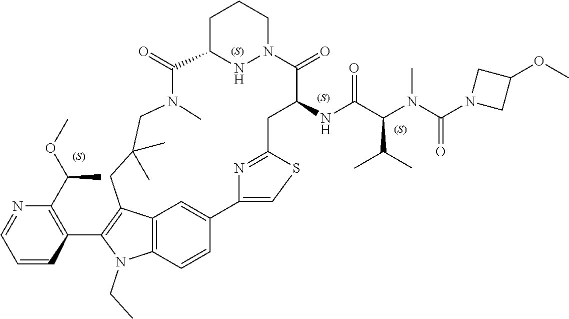 |
| AA342 | 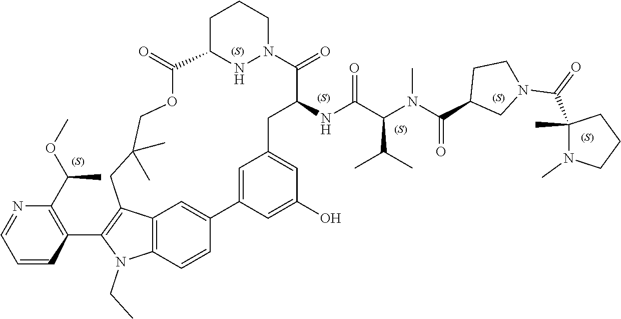 |
| AA343 | 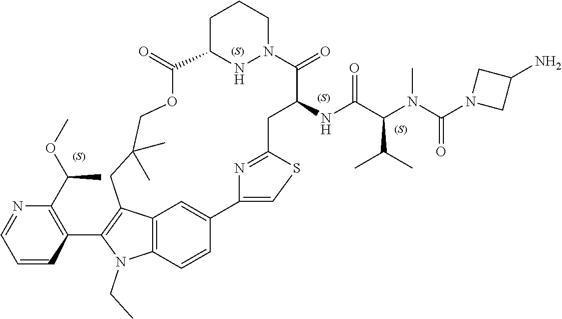 |
| AA344 | 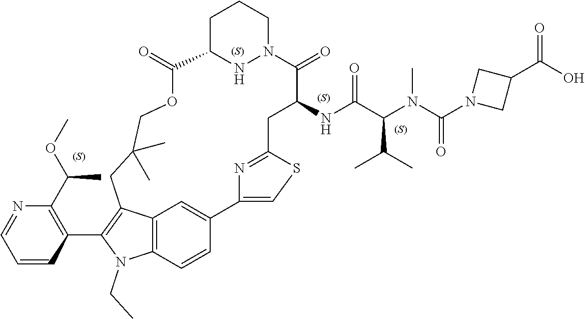 |
| AA345 | 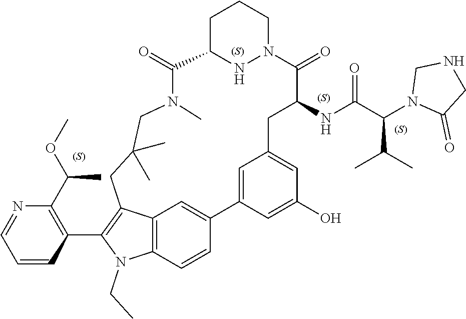 |
| AA346 | 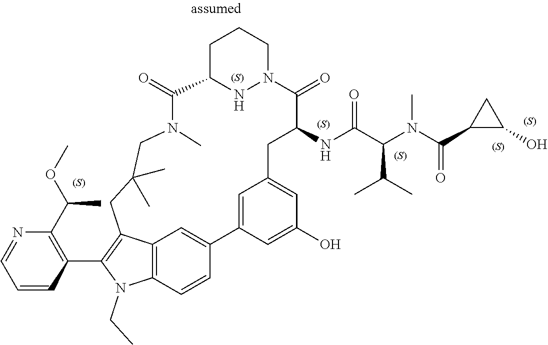 |
| AA347 | 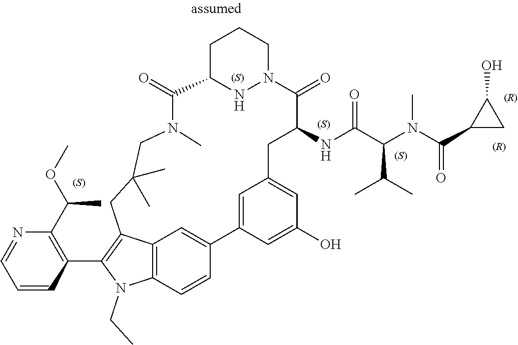 |
| AA348 | 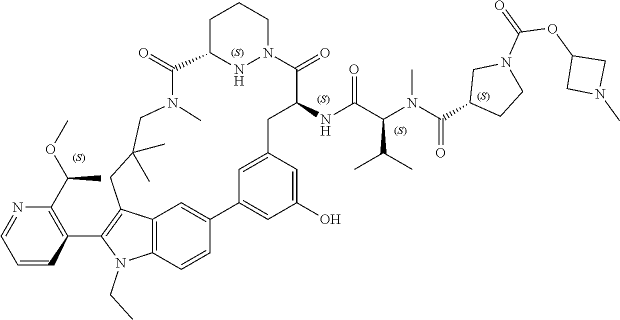 |
| AA349 | 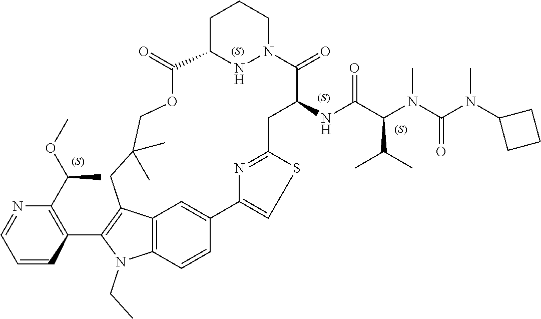 |
| AA350 | 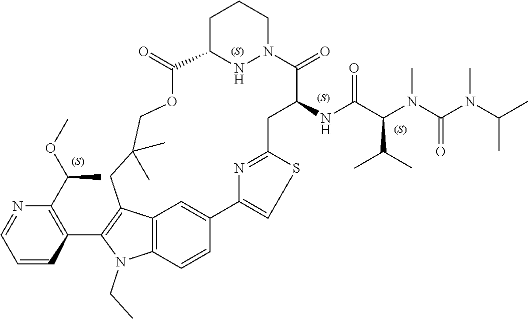 |
| AA351 | 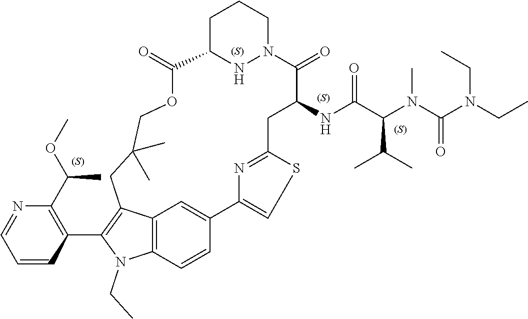 |
| AA352 | 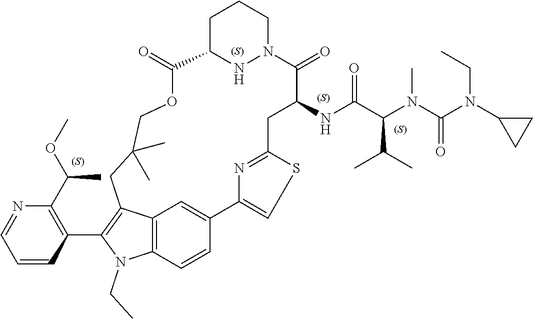 |
| AA353 | 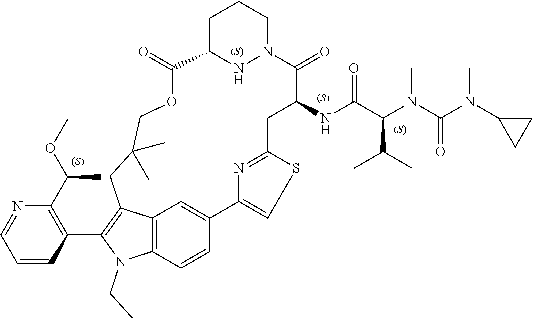 |
| AA354 | 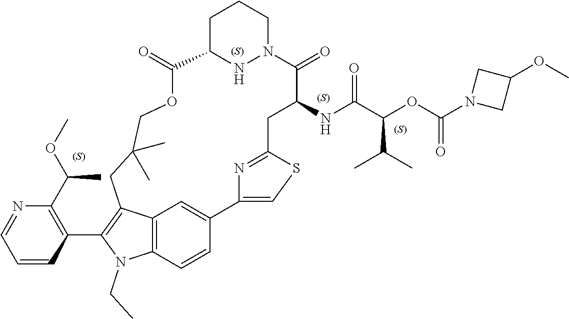 |
| AA355 | 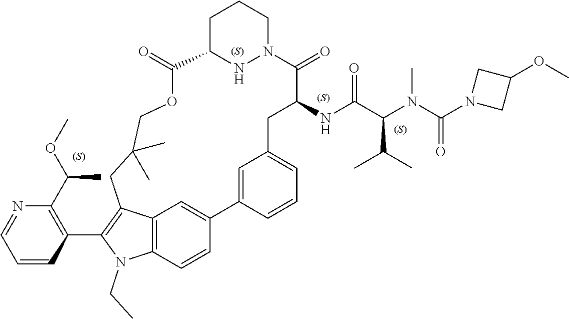 |
| AA356 | 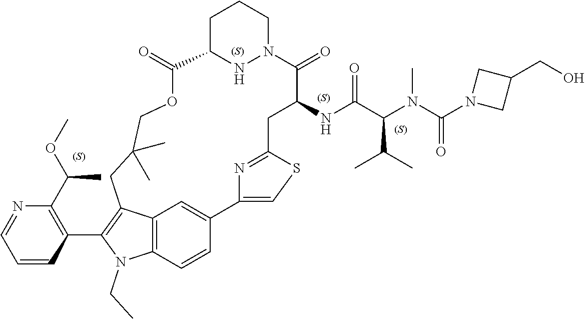 |
| AA357 | 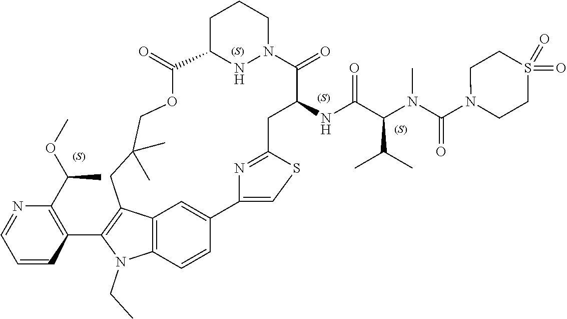 |
| AA358 | 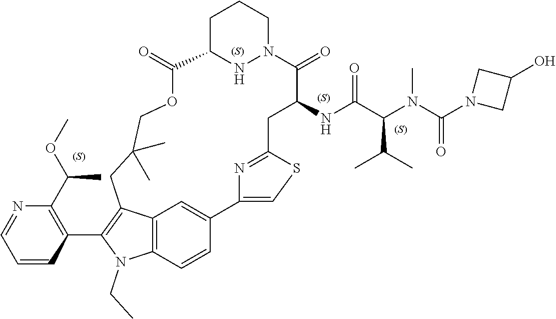 |
| AA359 | 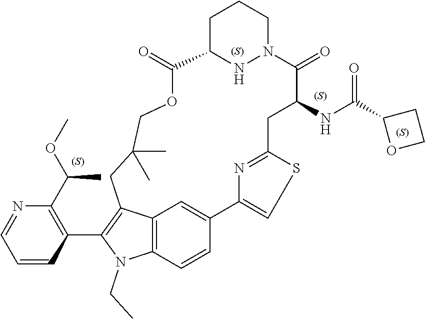 |
| AA360 | 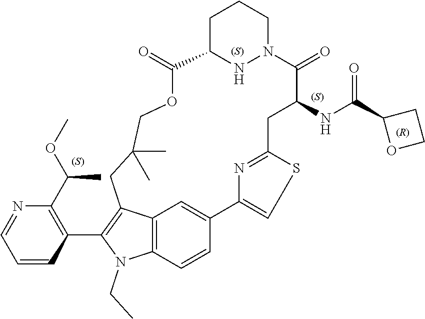 |
| AA361 | 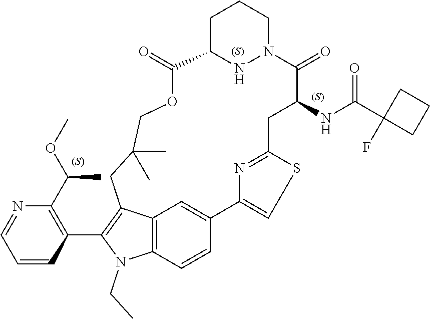 |
| AA362 | 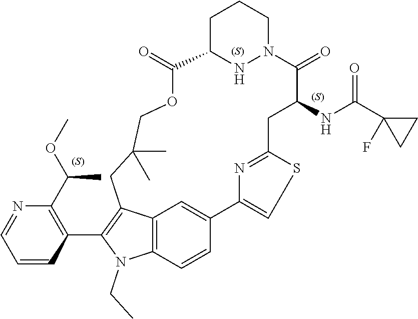 |
| AA363 | 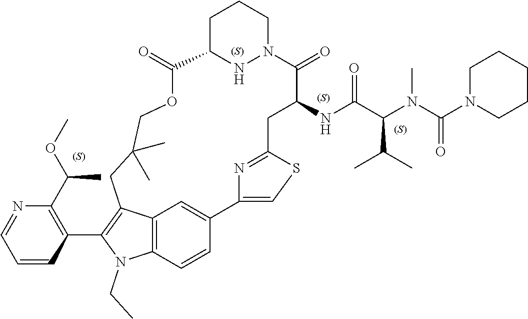 |
| AA364 | 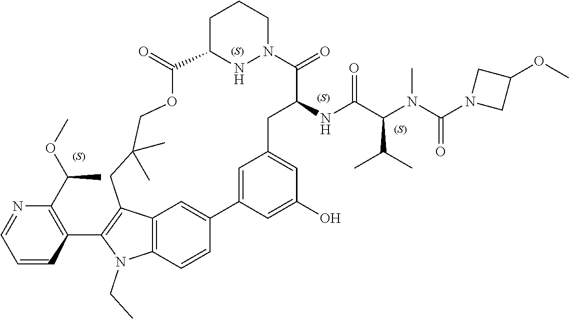 |
| AA365 | 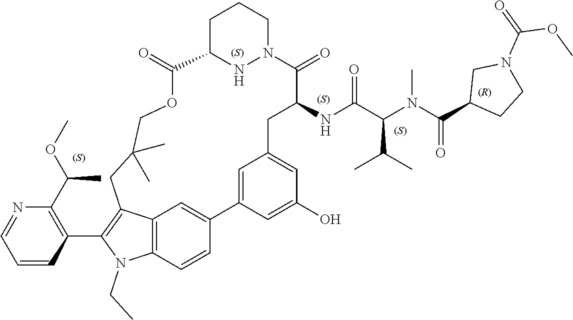 |
| AA366 | 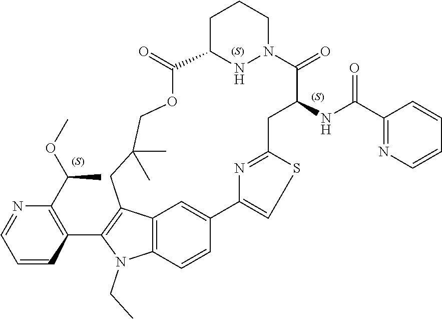 |
| AA367 | 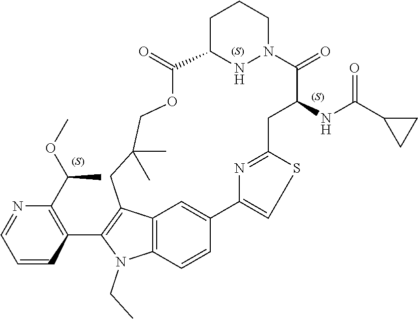 |
| AA368 | 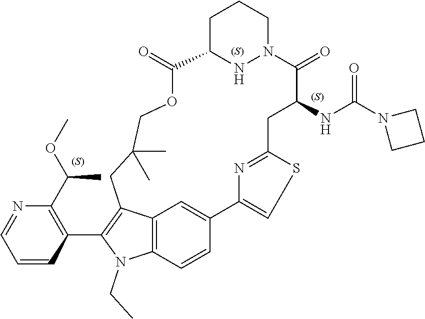 |
| AA369 | 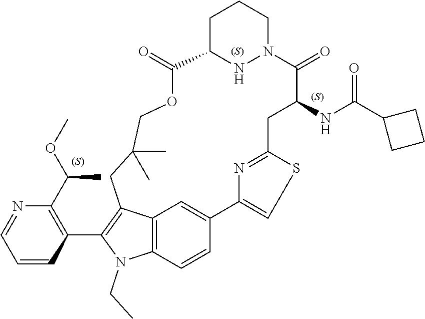 |
| AA370 | 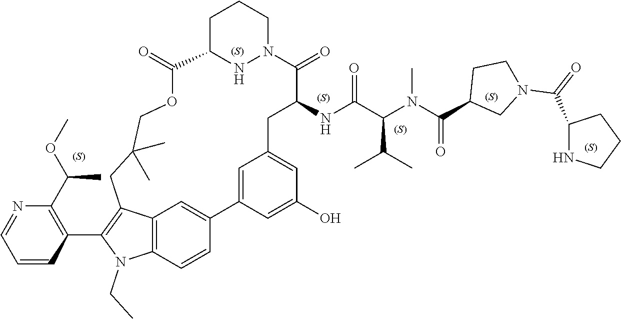 |
| AA371 | 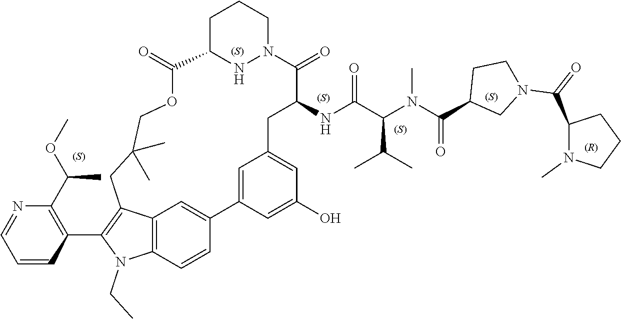 |
| AA372 | 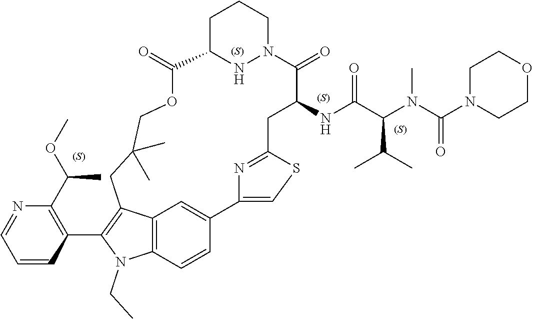 |
| AA373 | 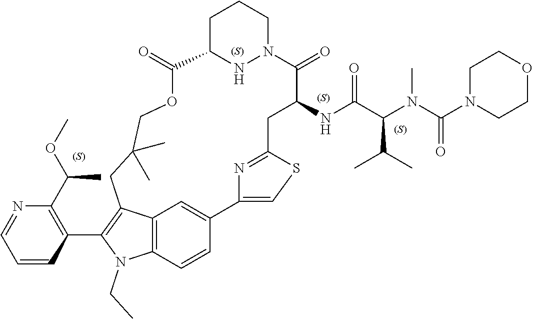 |
| AA374 | 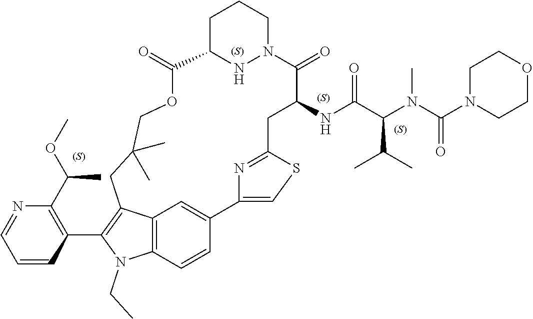 |
| AA375 | 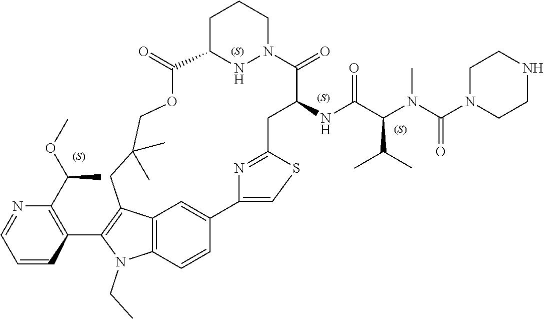 |
| AA376 | 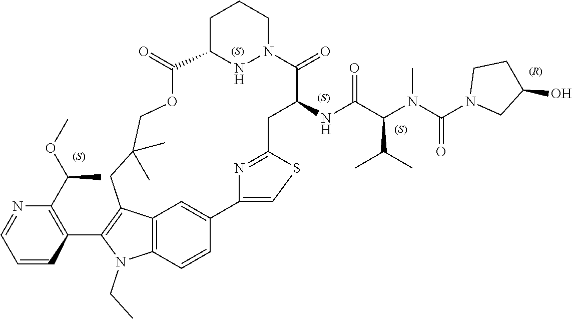 |
| AA377 | 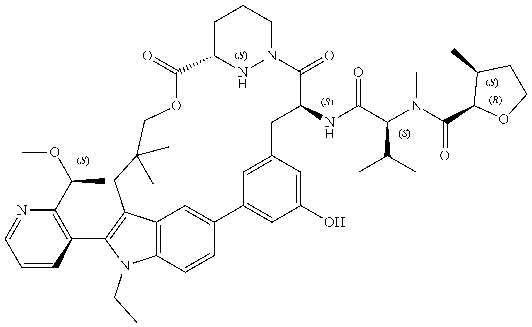 |
| AA378 | 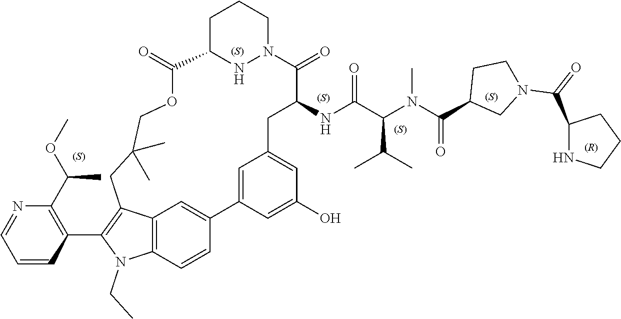 |
| AA379 | 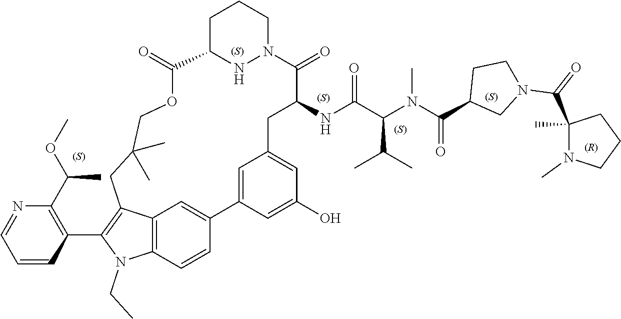 |
| AA380 | 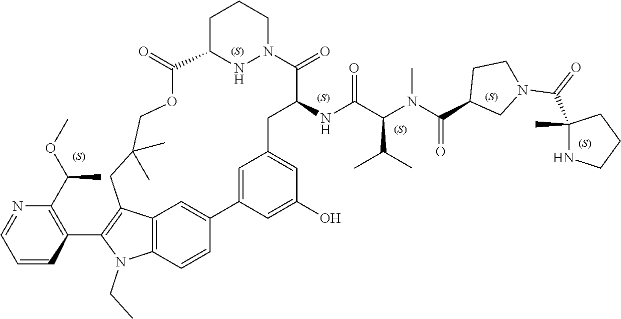 |
| AA381 | 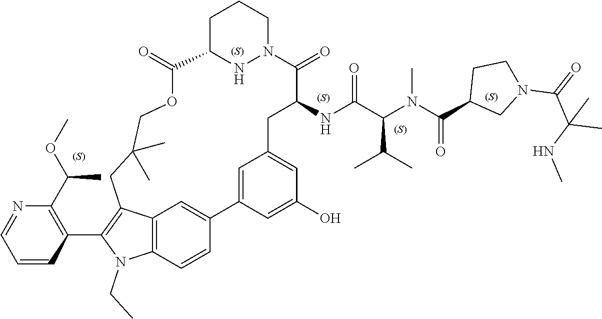 |
| AA382 | 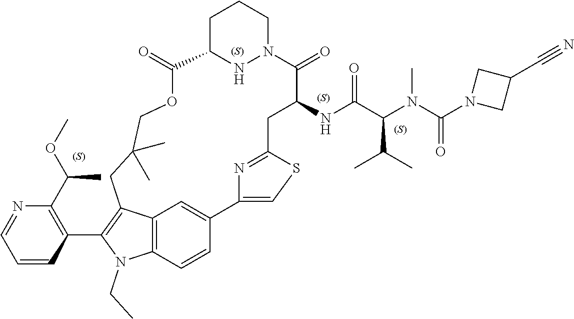 |
| AA383 | 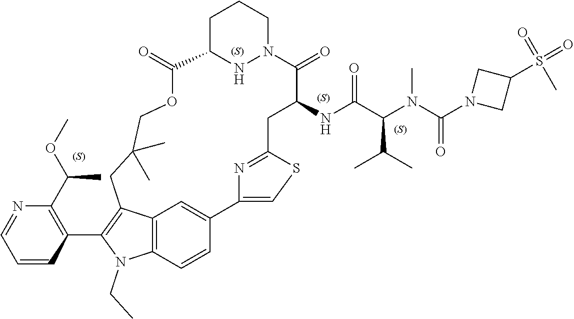 |
| AA384 | 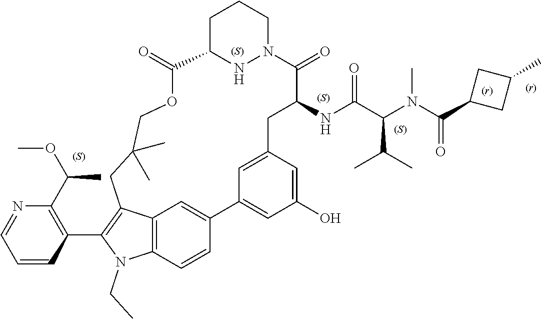 |
| AA385 | 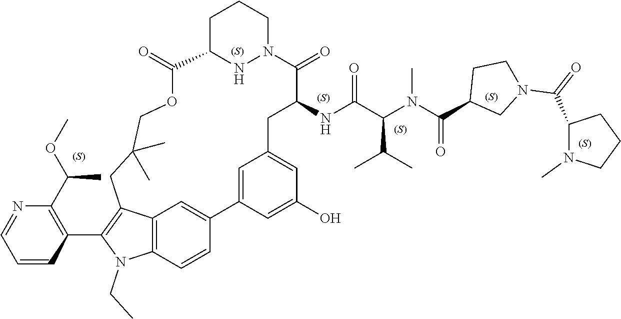 |
| AA386 | 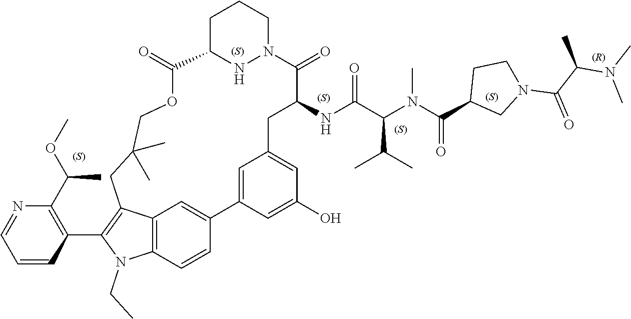 |
| AA387 | 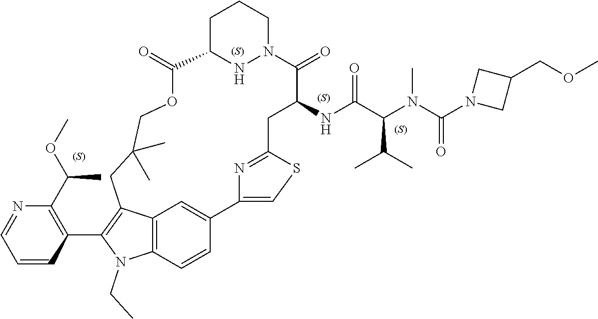 |
| AA388 | 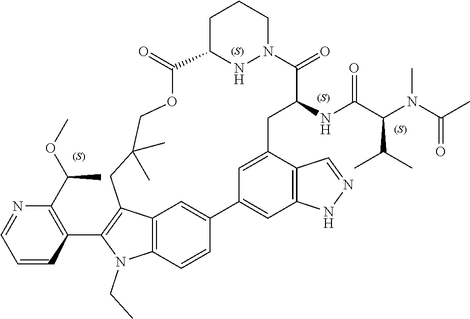 |
| AA389 | 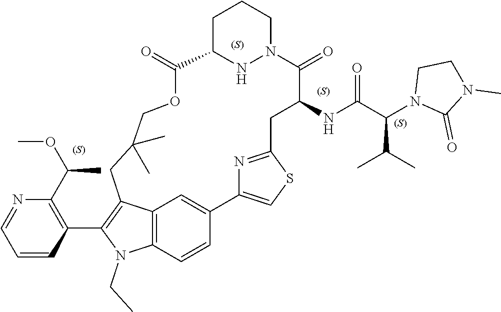 |
| AA391 | 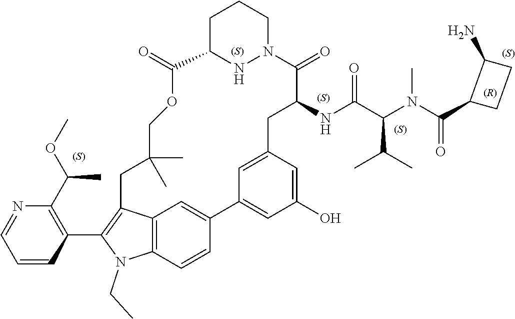 |
| AA392 | 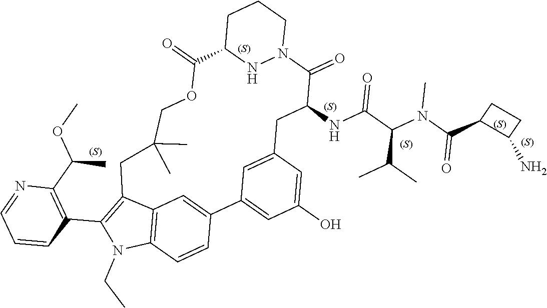 |
| AA393 | 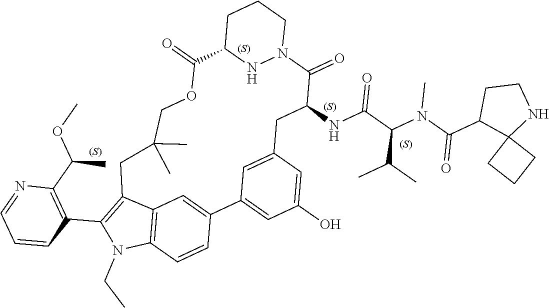 |
| AA394 | 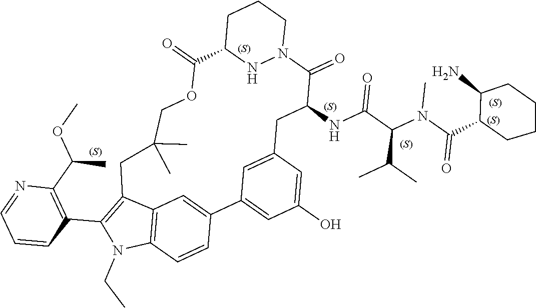 |
| AA395 | 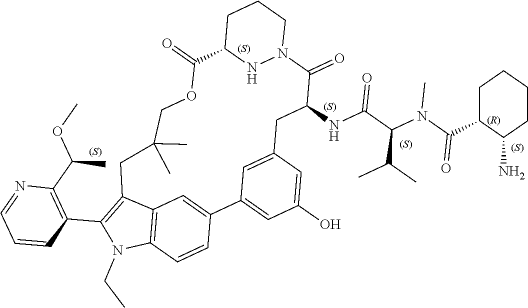 |
| AA396 | 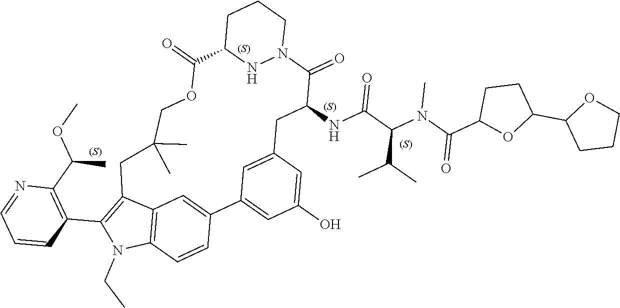 |
| AA397 | 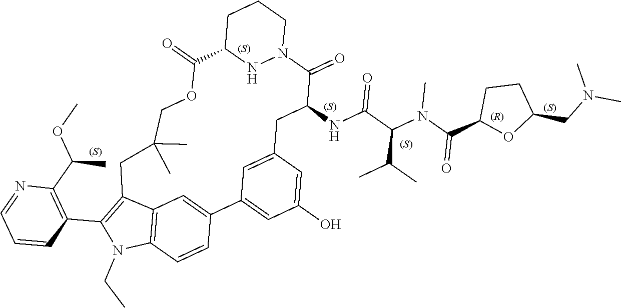 |
| AA398 | 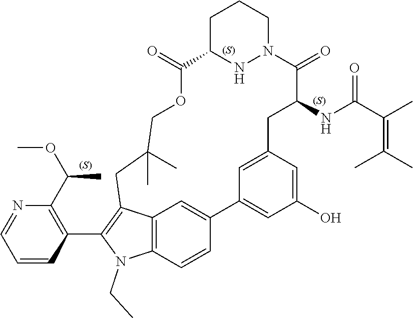 |
| AA399 | 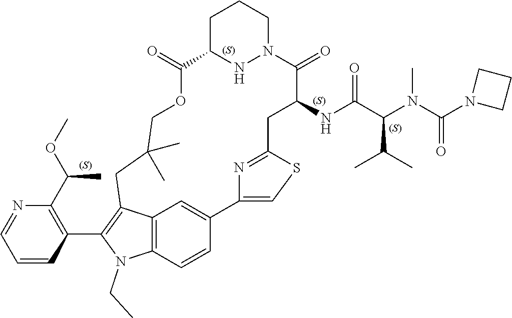 |
| AA400 | 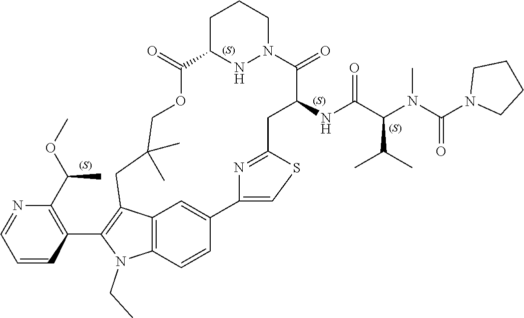 |
| AA401 | 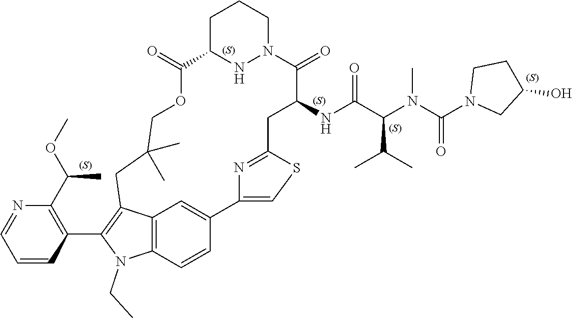 |
| AA402 | 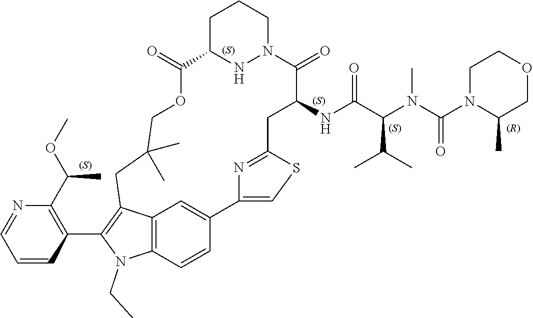 |
| AA403 | 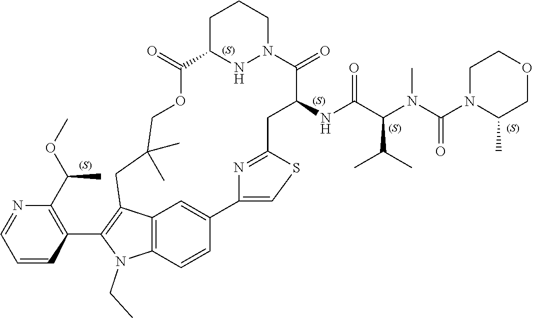 |
| AA404 | 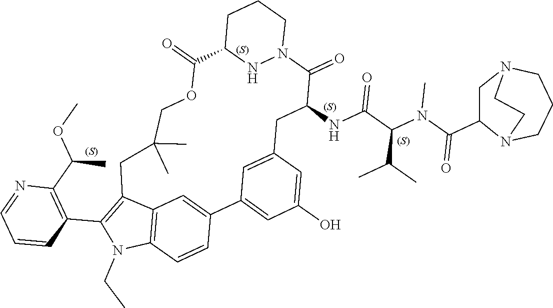 |
| AA405 | 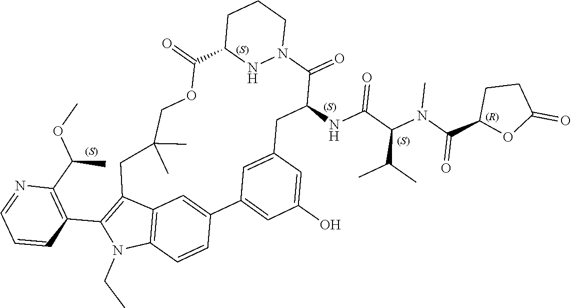 |
| AA406 | 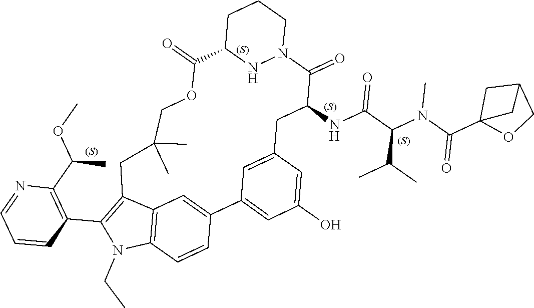 |
| AA407 | 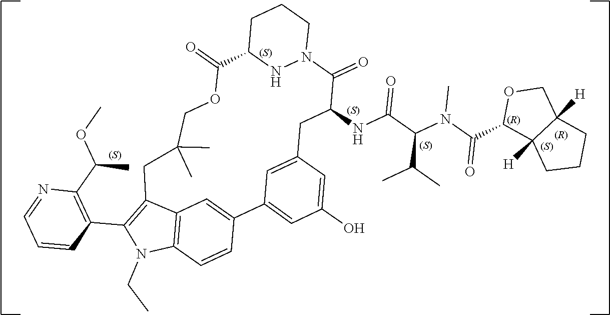 |
| AA408 | 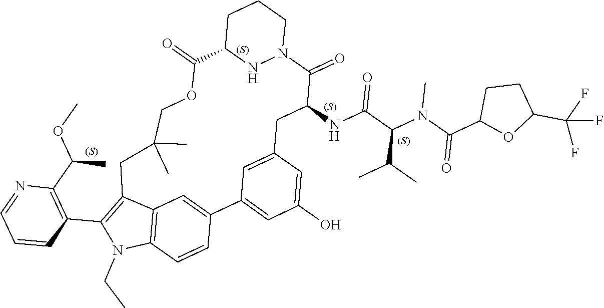 |
| AA409 | 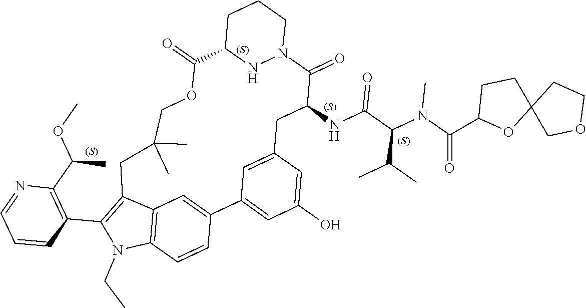 |
| AA410 | 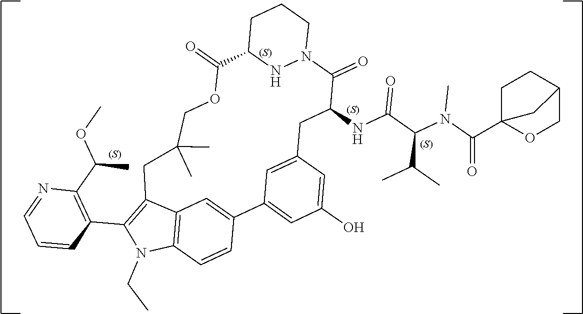 |
| AA411 | 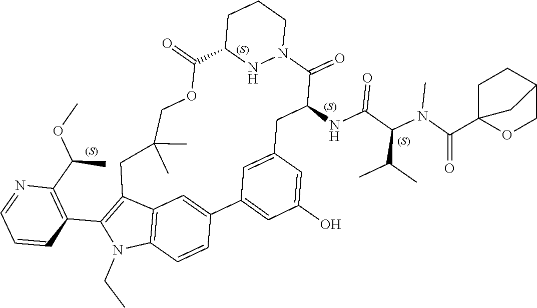 |
| AA412 | 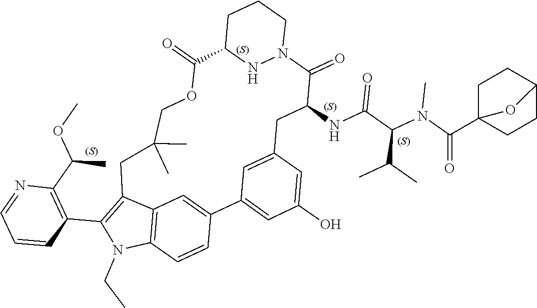 |
| AA413 | 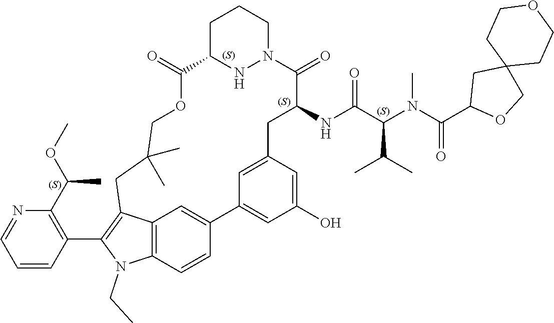 |
| AA414 | 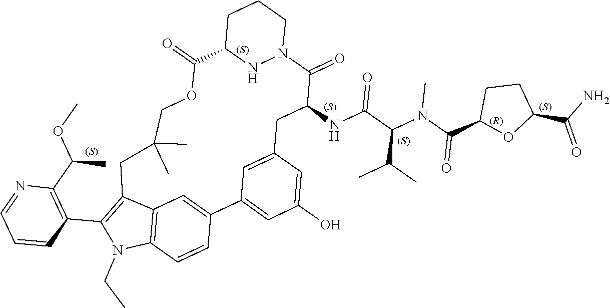 |
| AA415 | 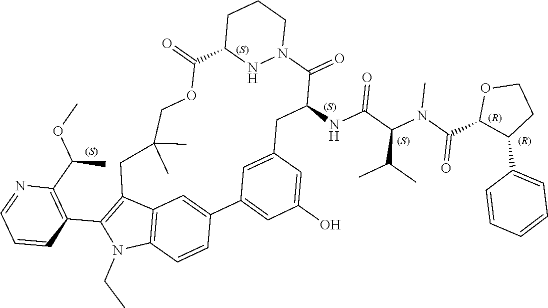 |
| AA416 | 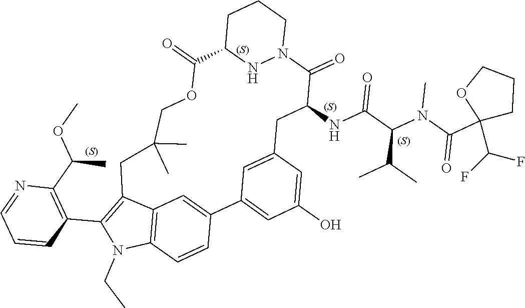 |
| AA417 | 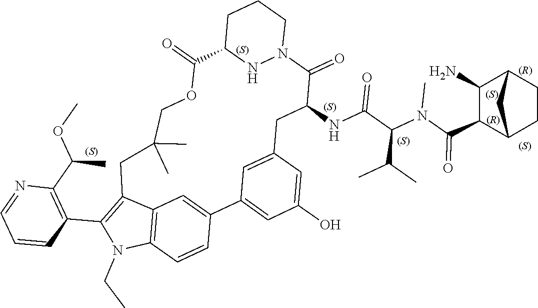 |
| AA418 | 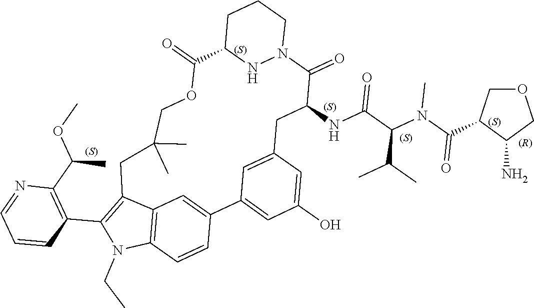 |
| AA419 | 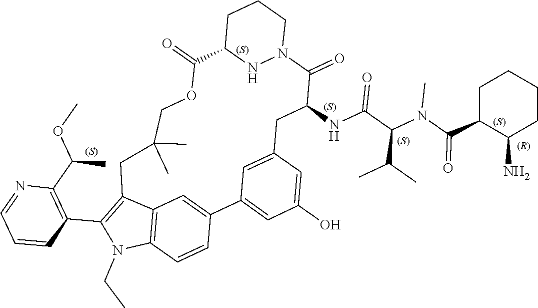 |
| AA420 | 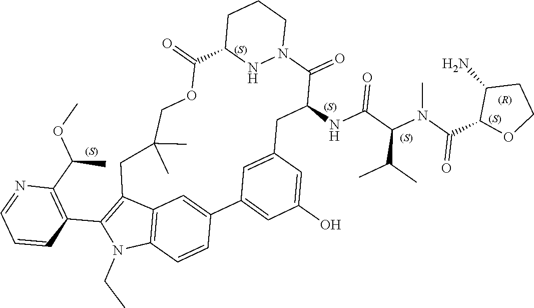 |
| AA421 | 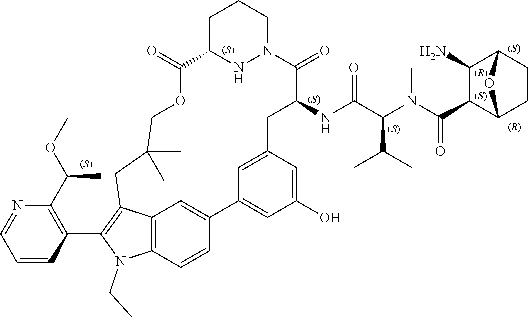 |
| AA422 | 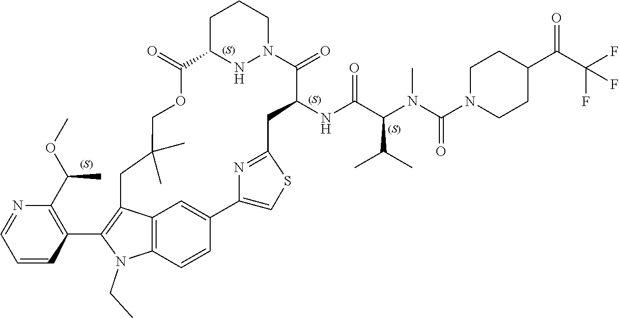 |
| AA423 | 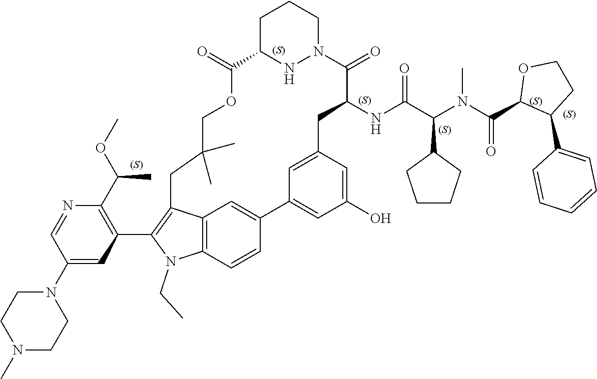 |
| AA424 | 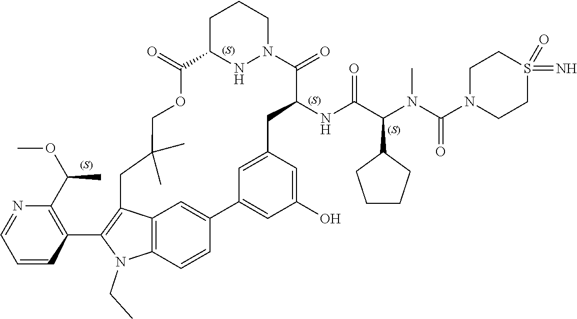 |
| AA425 | 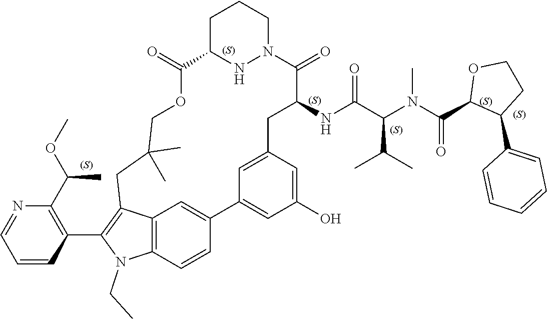 |
| AA426 | 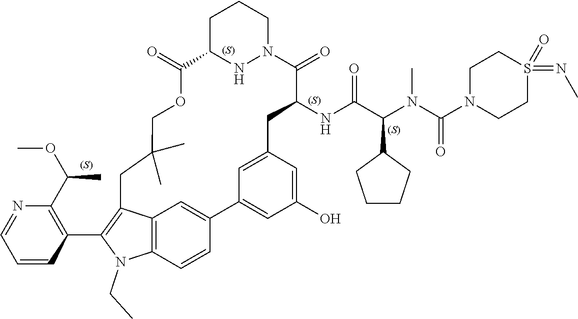 |
| AA427 | 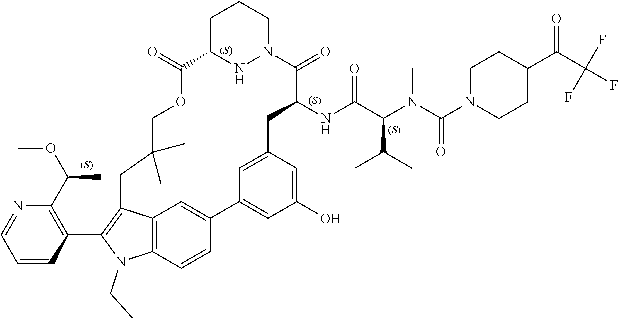 |
| AA428 | 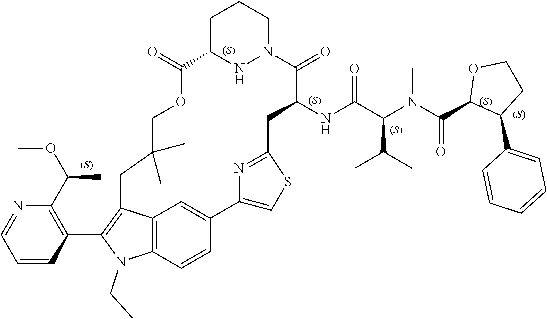 |
| AA429 | 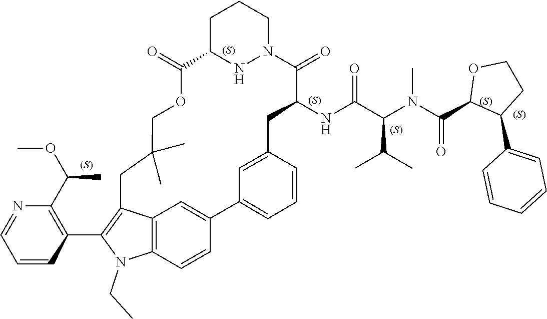 |
| AA430 | 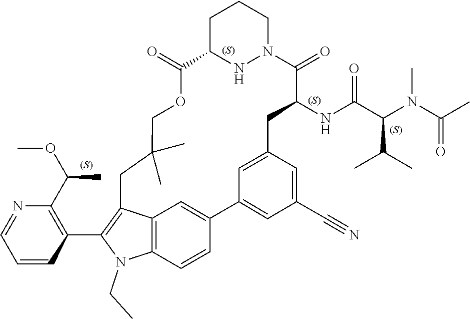 |
| AA431 | 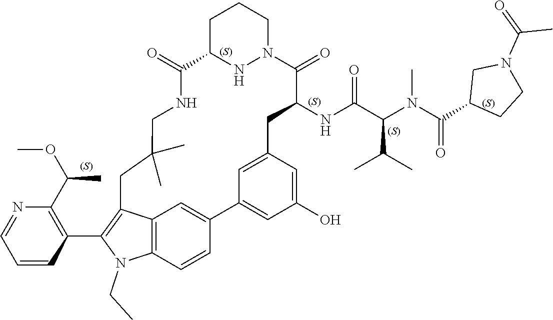 |
| AA432 | 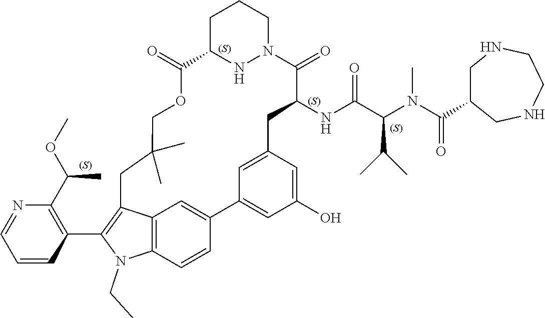 |
| AA433 | 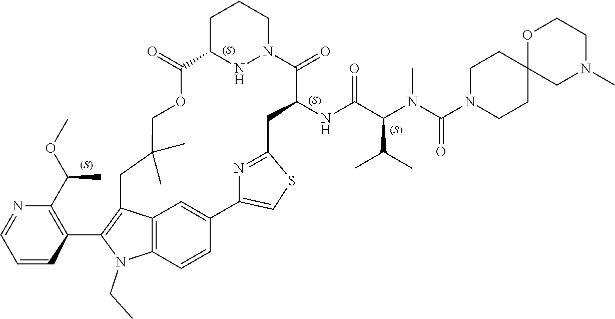 |
| AA434 | 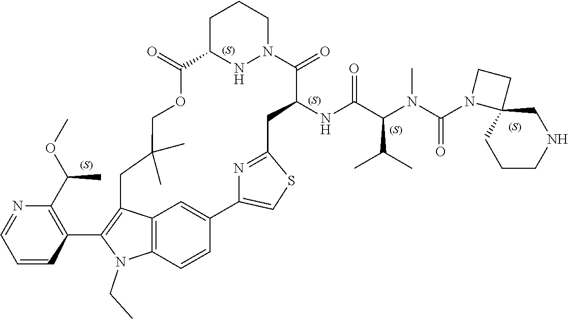 |
| AA435 | 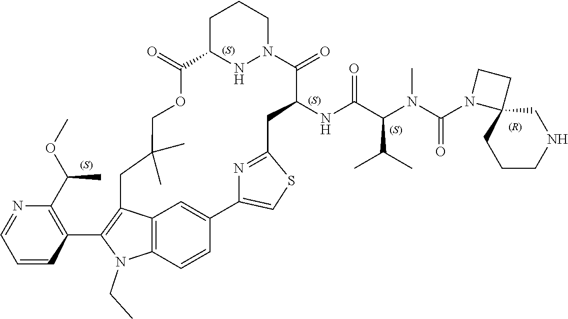 |
| AA436 | 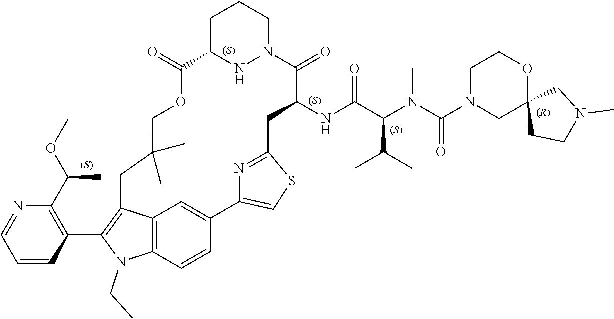 |
| AA437 | 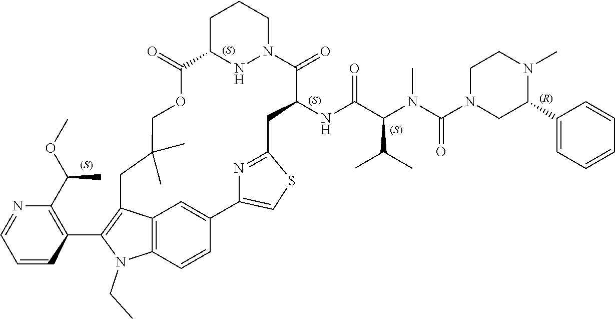 |
| AA438 | 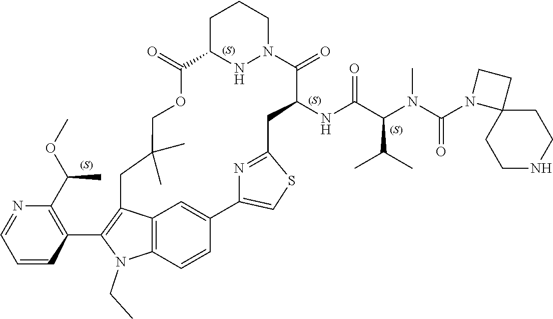 |
| AA439 | 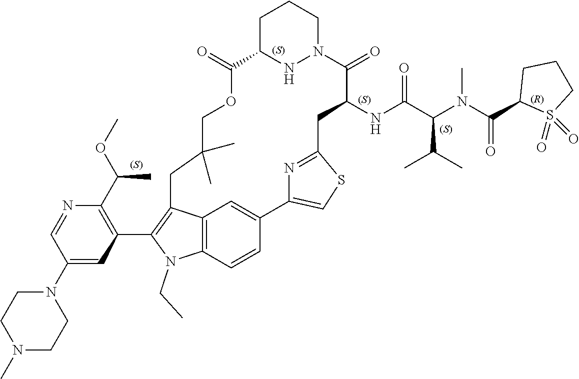 |
| AA440 | 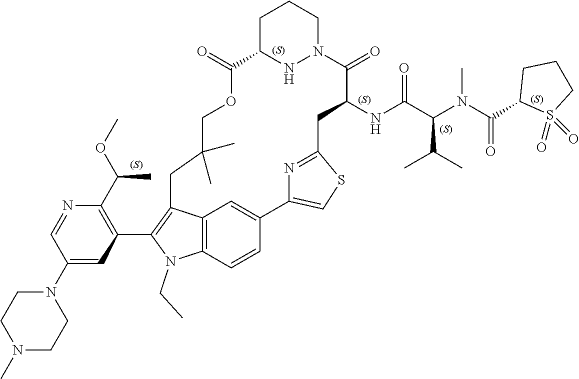 |
| AA441 | 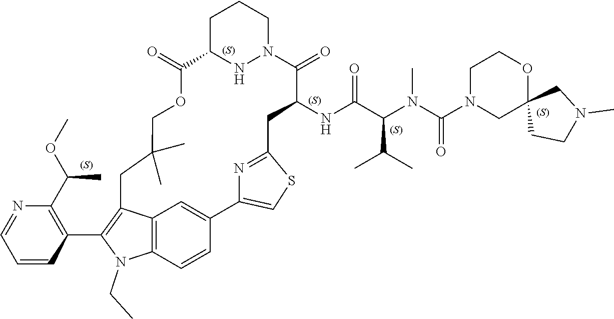 |
| AA442 | 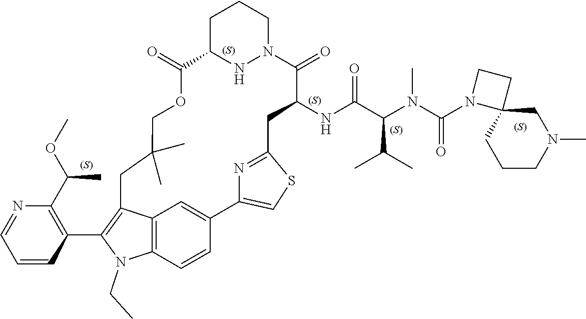 |
| AA443 | 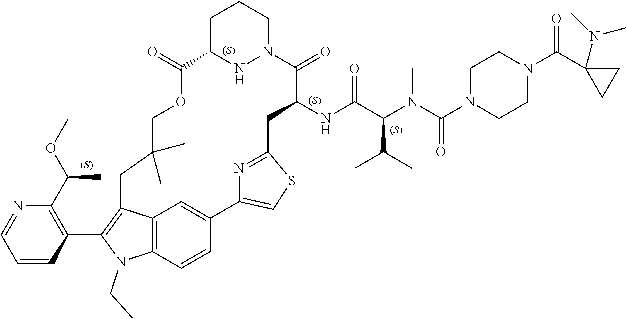 |
| AA444 | 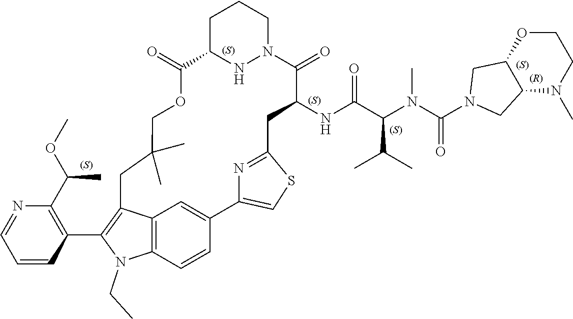 |
| AA445 | 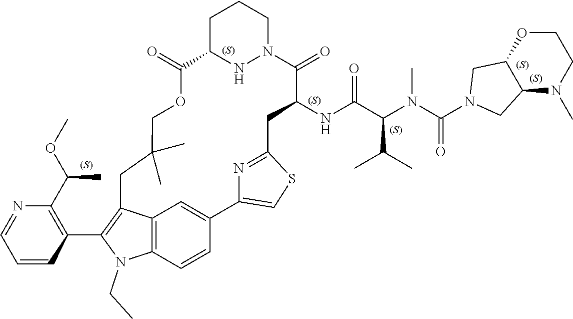 |
| AA446 | 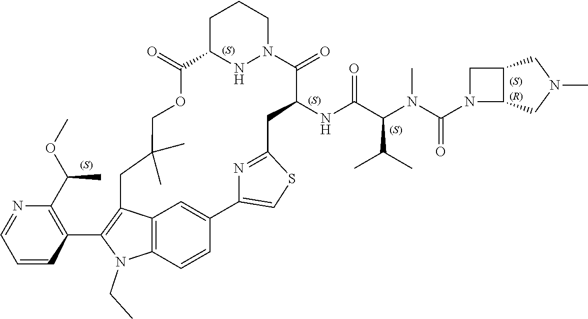 |
| AA447 | 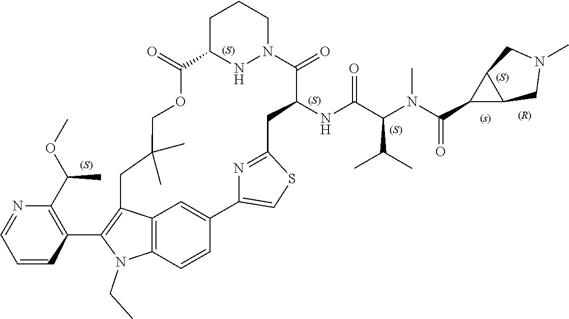 |
| AA448 | 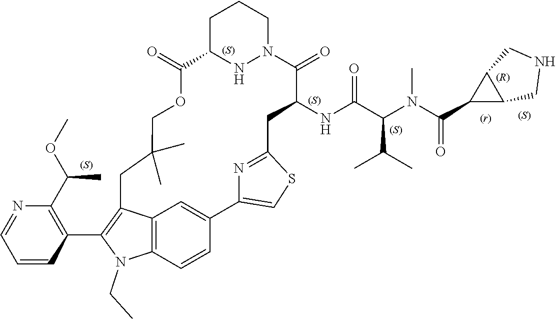 |
| AA449 | 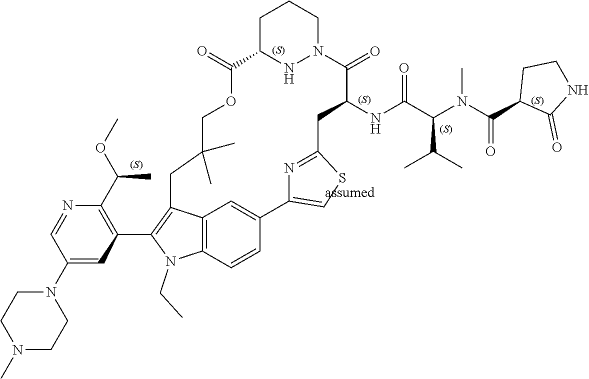 |
| AA450 | 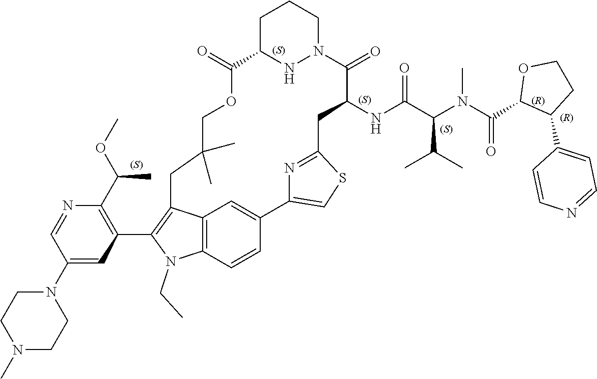 |
| AA451 | 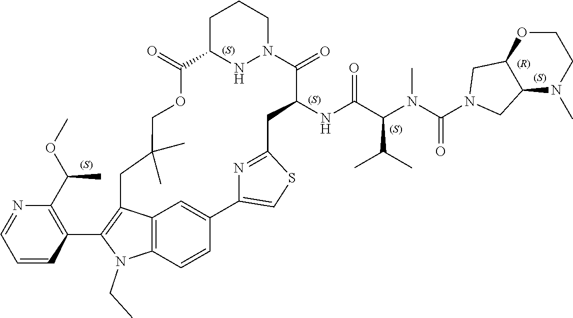 |
| AA452 | 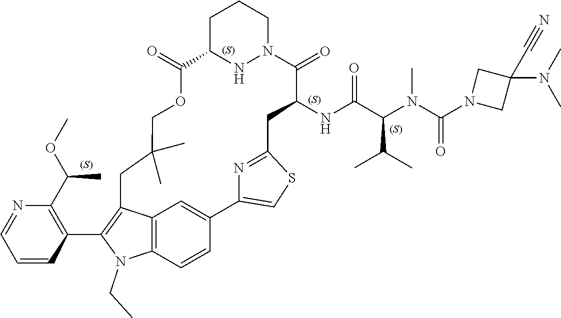 |
| AA453 | 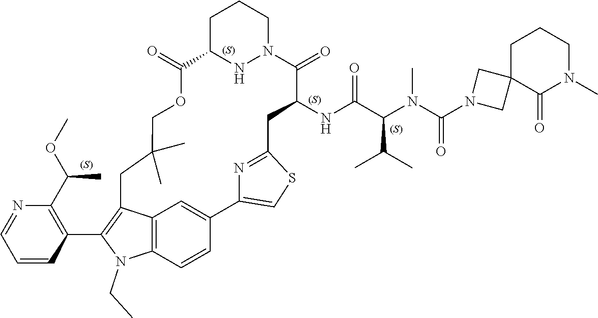 |
| AA454 | 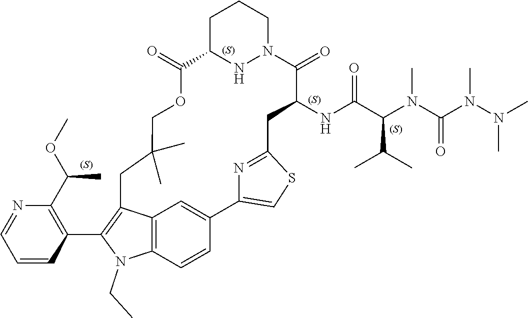 |
| AA455 | 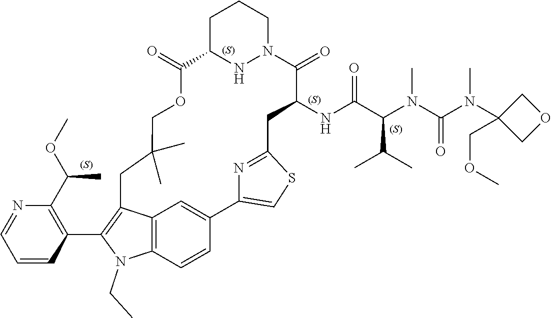 |
| AA456 | 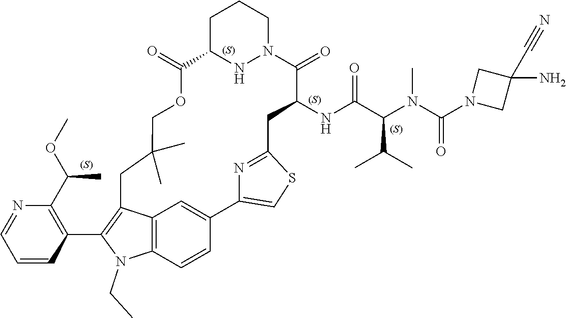 |
| AA457 | 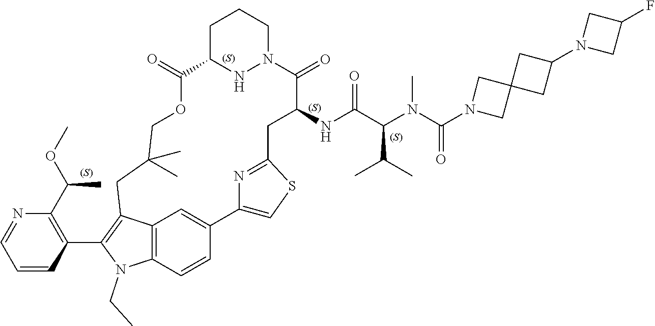 |
| AA458 | 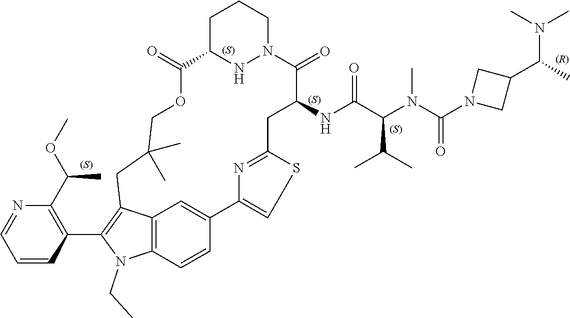 |
| AA459 | 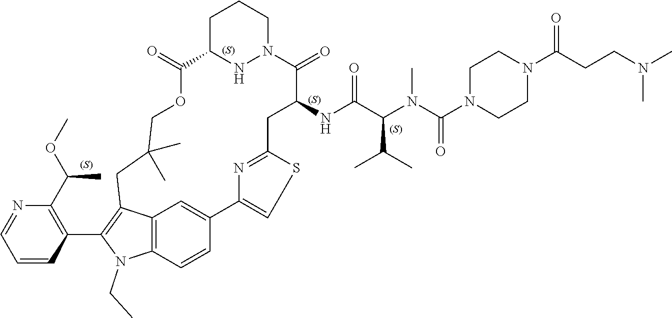 |
| AA460 | 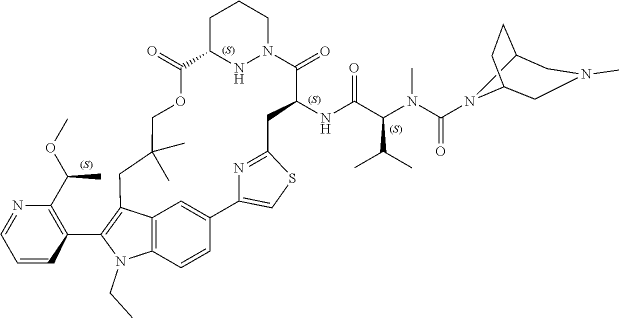 |
| AA461 | 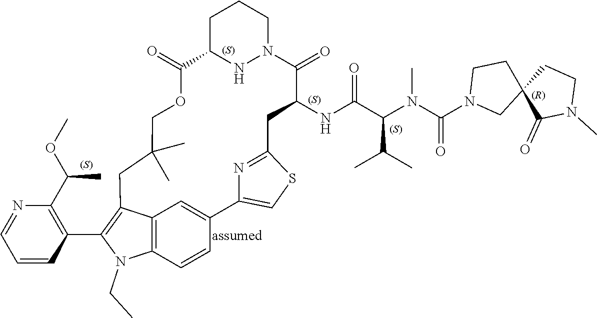 |
| AA462 | 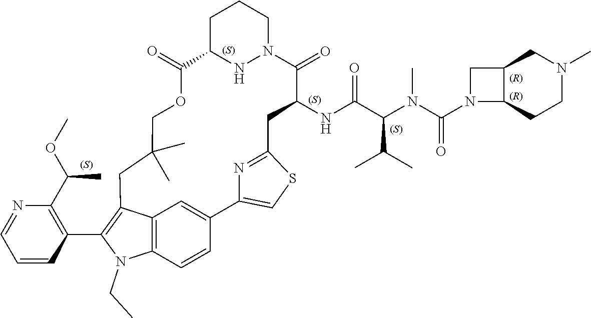 |
| AA463 | 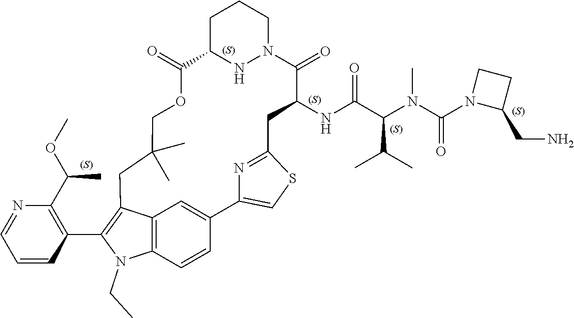 |
| AA464 | 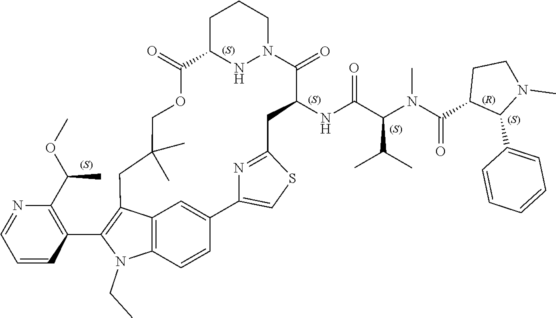 |
| AA465 | 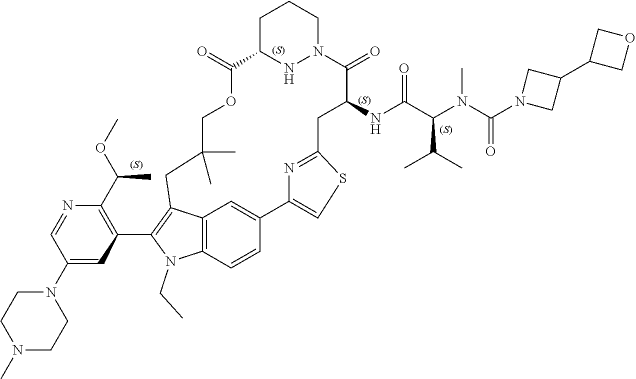 |
| AA466 | 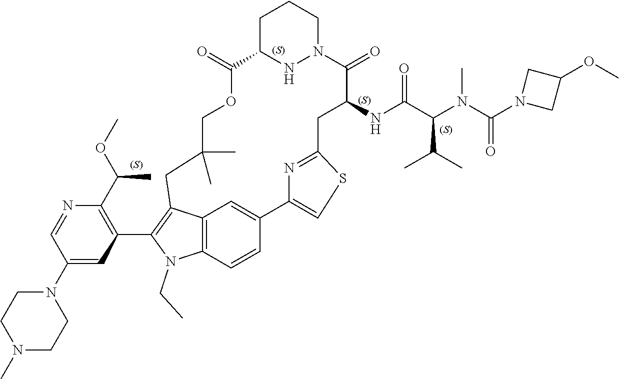 |
| AA467 | 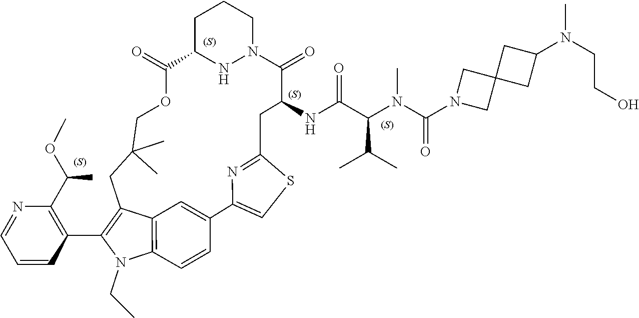 |
| AA468 | 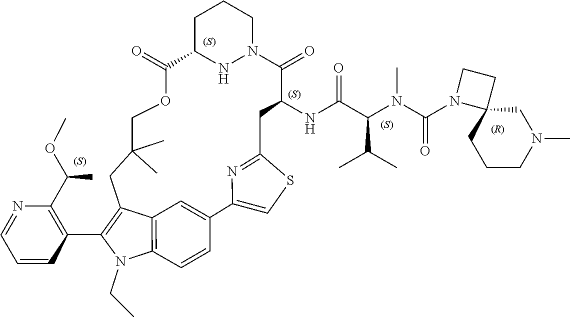 |
| AA469 | 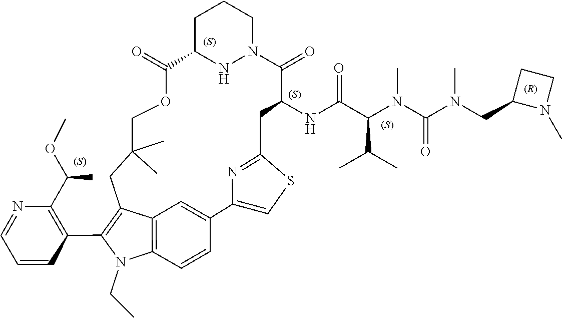 |
| AA470 | 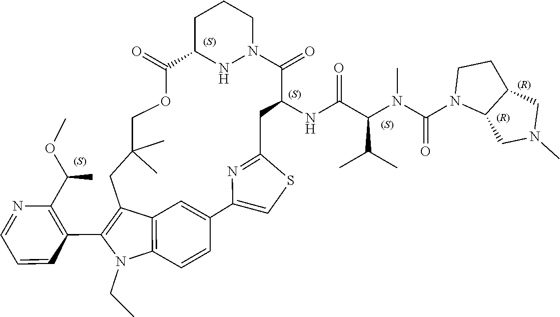 |
| AA471 | 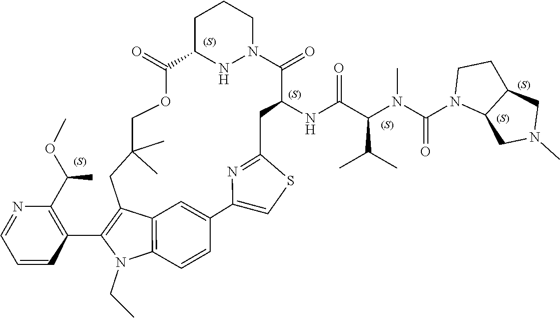 |
| AA472 | 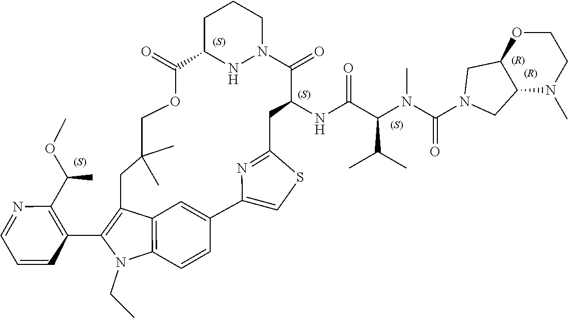 |
| AA473 | 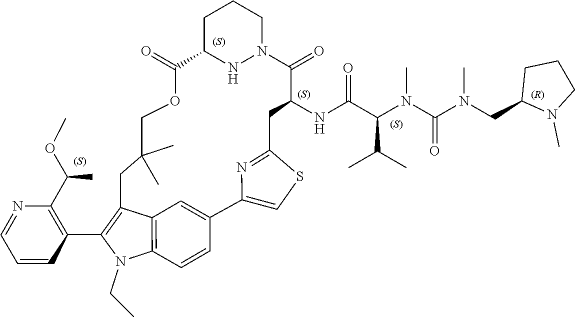 |
| AA474 | 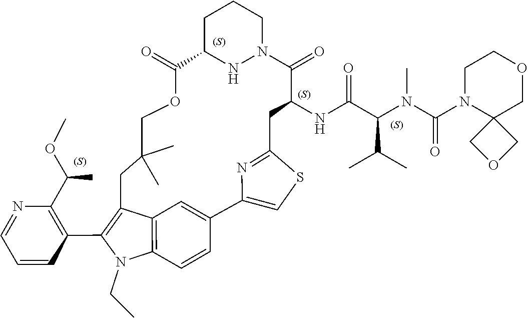 |
| AA475 | 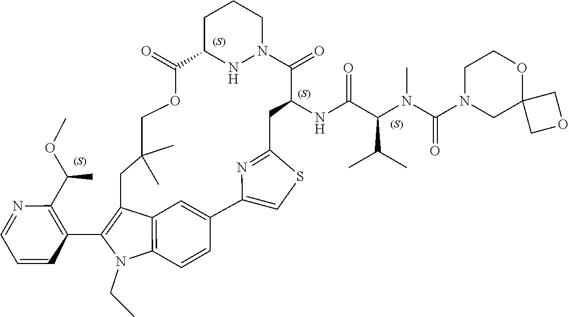 |
| AA476 | 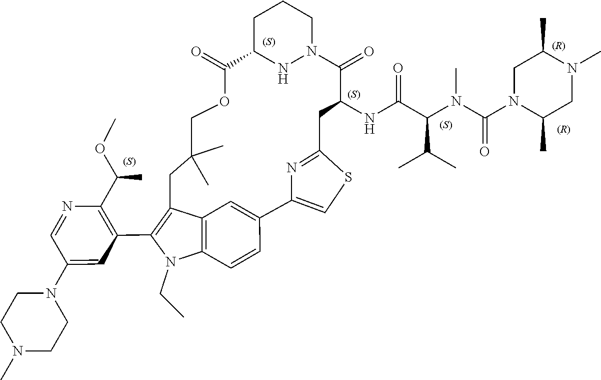 |
| AA477 | 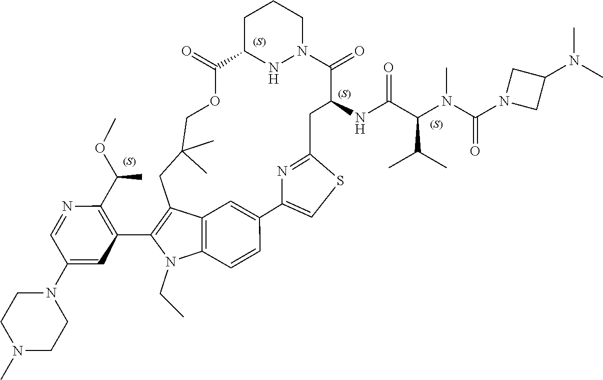 |
| AA478 | 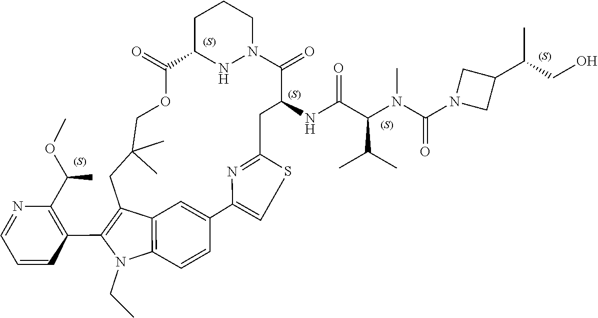 |
| AA479 | 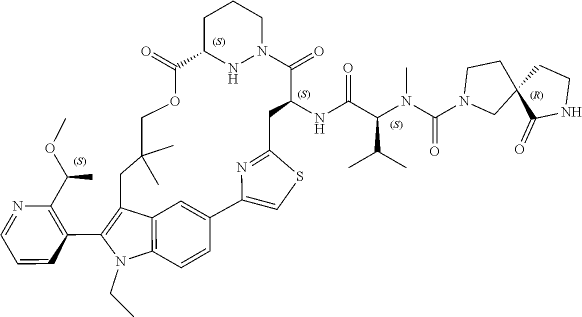 |
| AA480 | 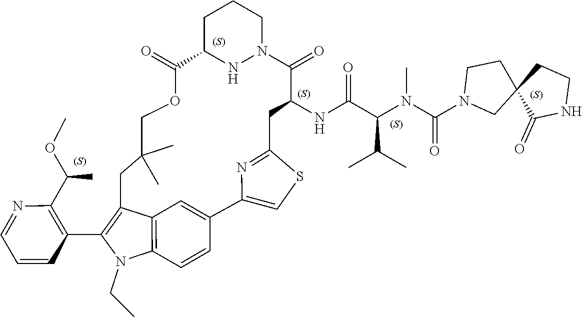 |
| AA481 | 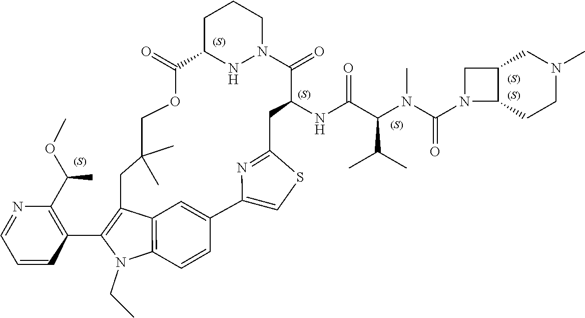 |
| AA482 | 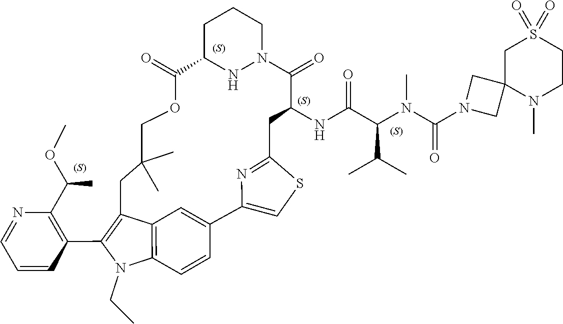 |
| AA483 | 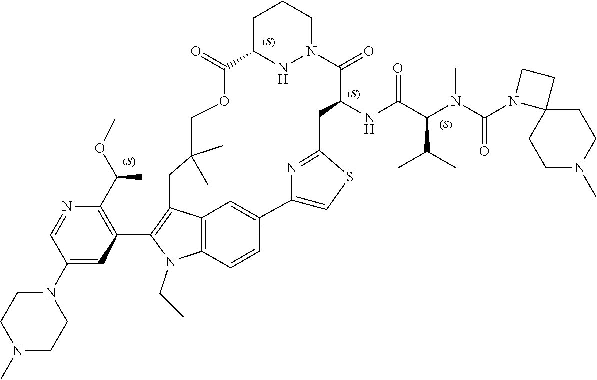 |
| AA484 | 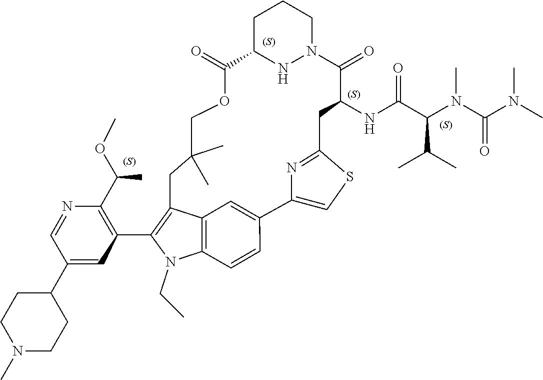 |
| AA485 | 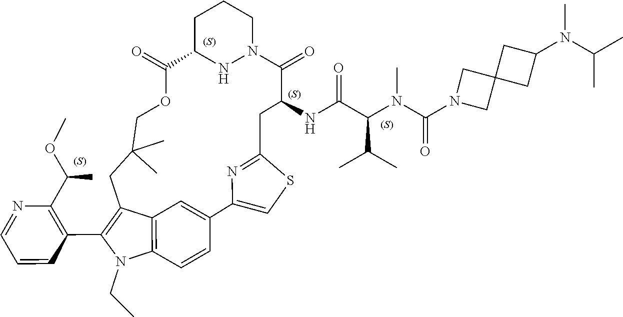 |
| AA486 | 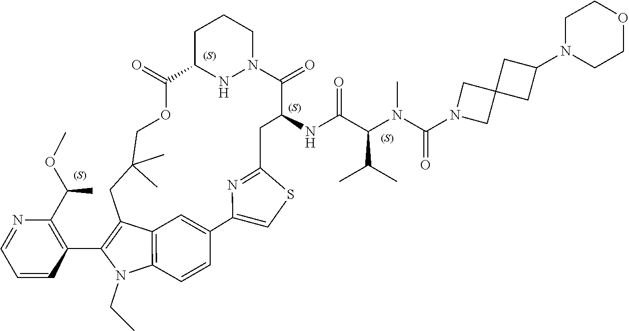 |
| AA487 | 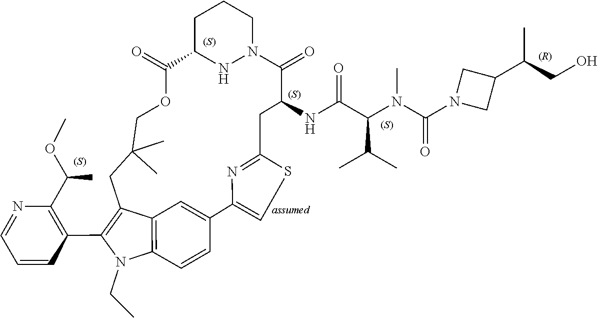 |
| AA488 | 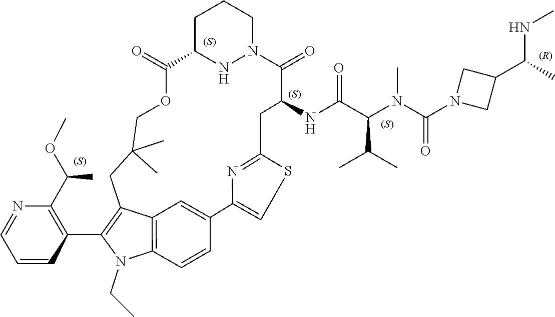 |
| AA489 | 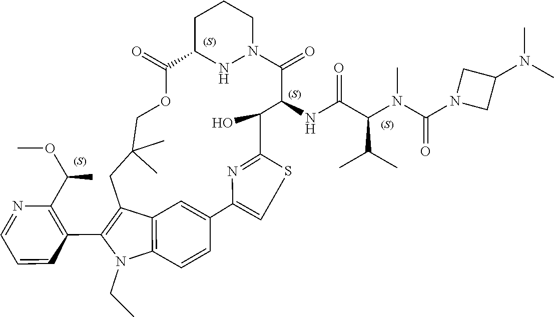 |
| AA490 | 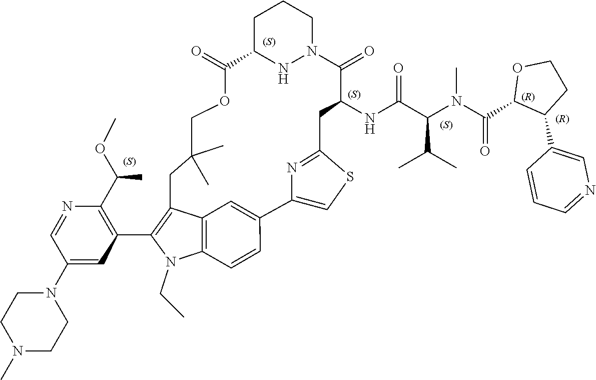 |
| AA491 | 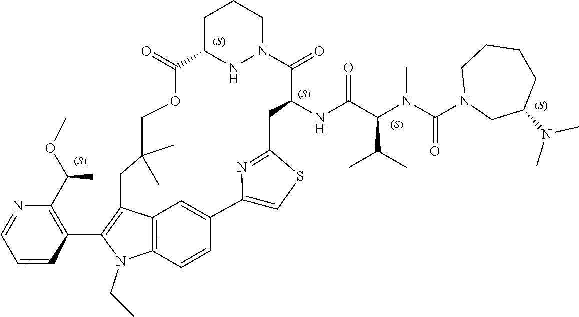 |
| AA492 | 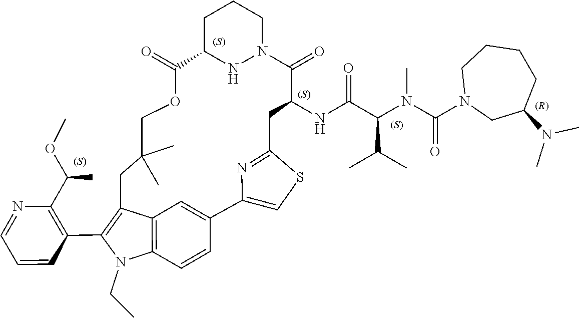 |
| AA493 | 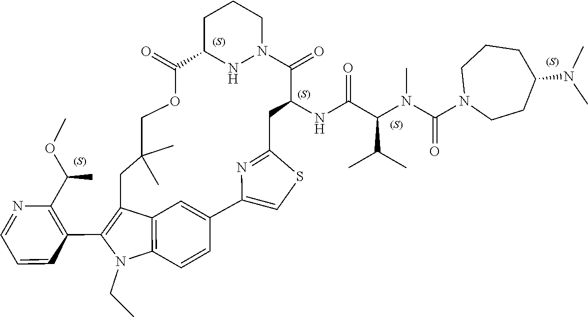 |
| AA494 | 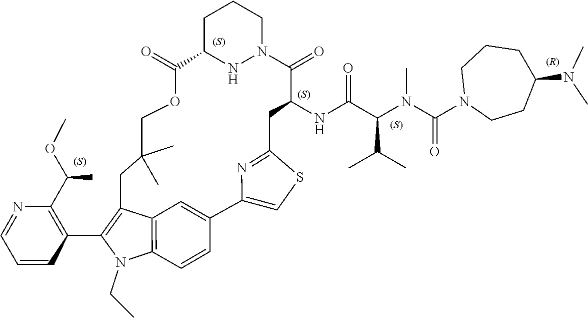 |
| AA495 | 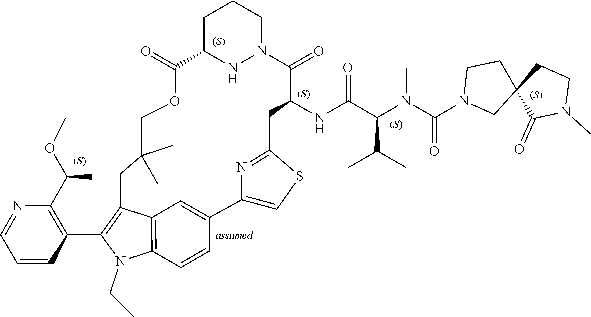 |
| AA496 | 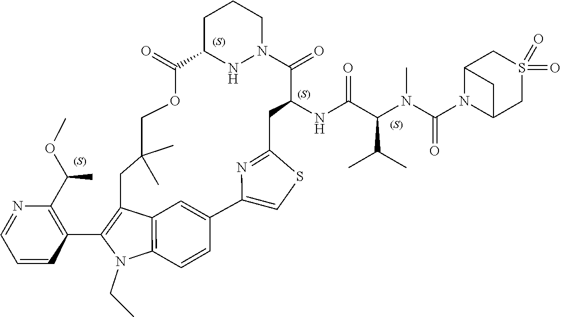 |
| AA497 | 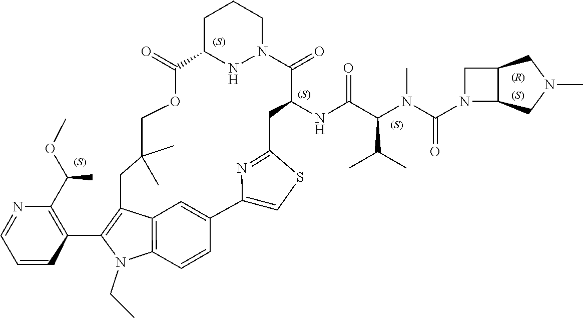 |
| AA498 | 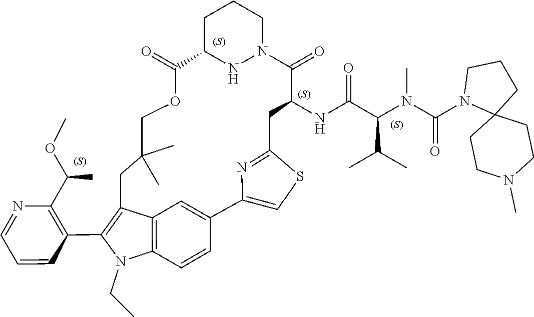 |
| AA499 | 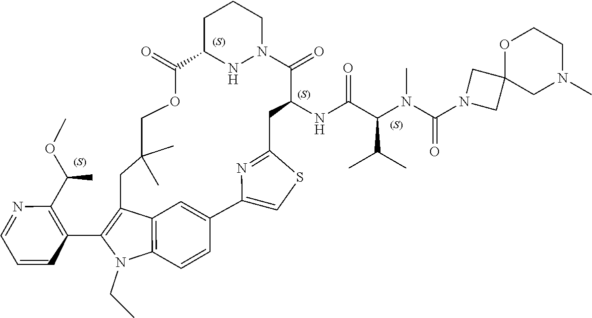 |
| AA500 | 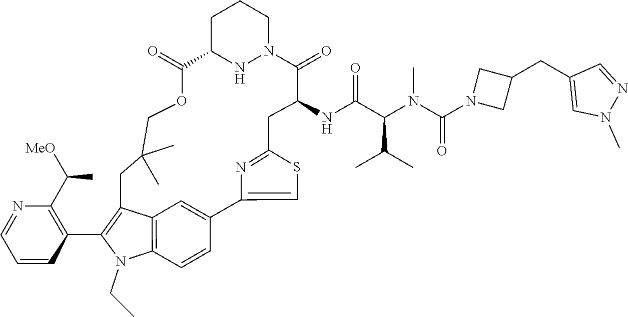 |
| AA501 | 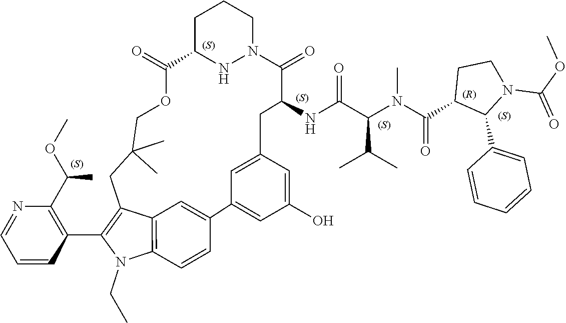 |
| AA502 | 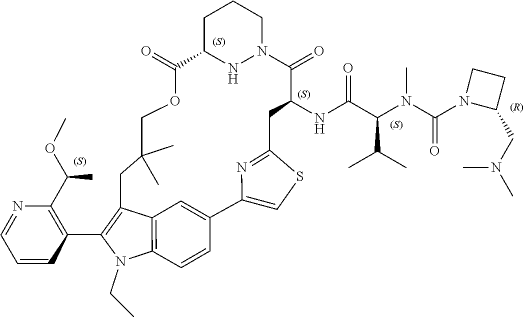 |
| AA503 | 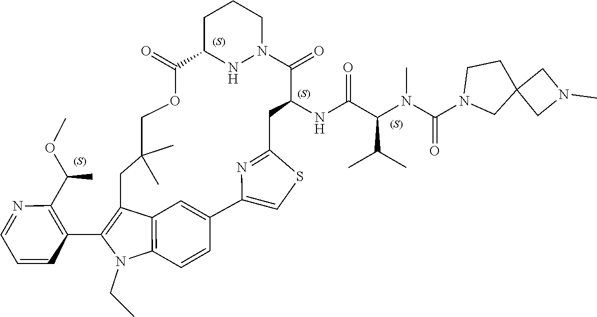 |
| AA504 | 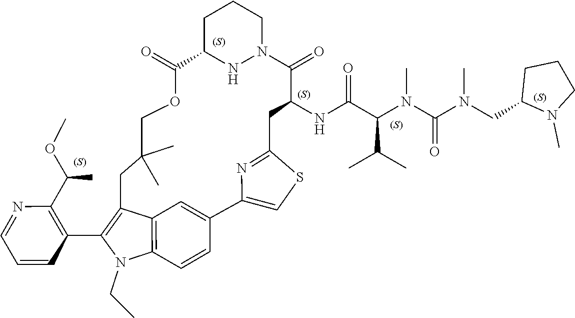 |
| AA505 | 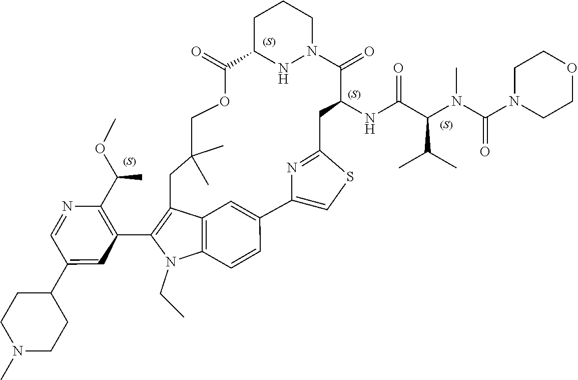 |
| AA506 | 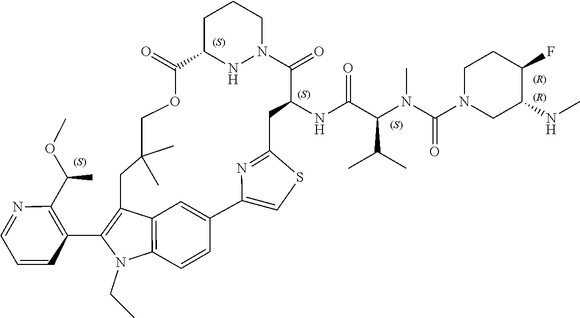 |
| AA507 | 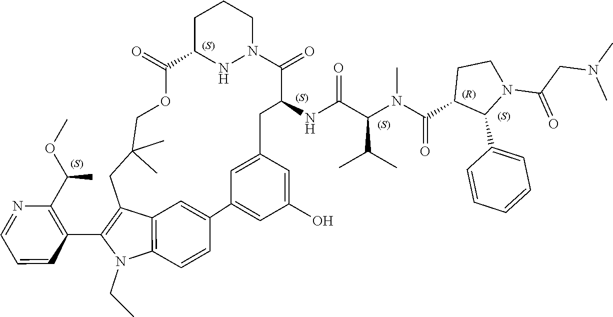 |
| AA508 | 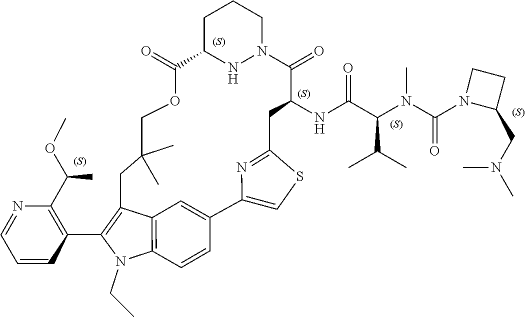 |
| AA509 | 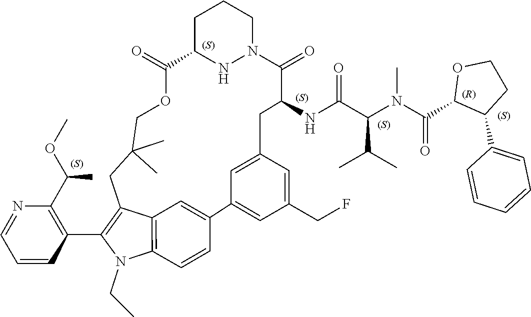 |
| AA510 | 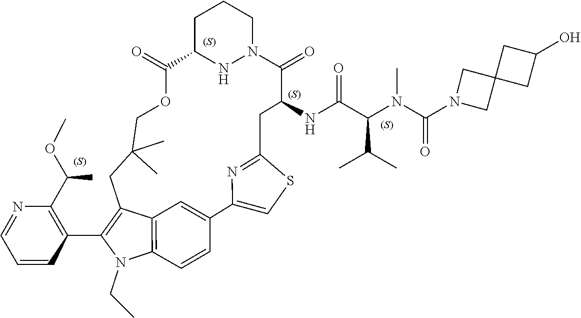 |
| AA511 | 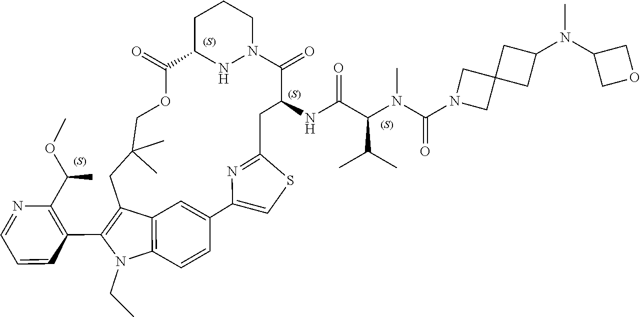 |
| AA512 | 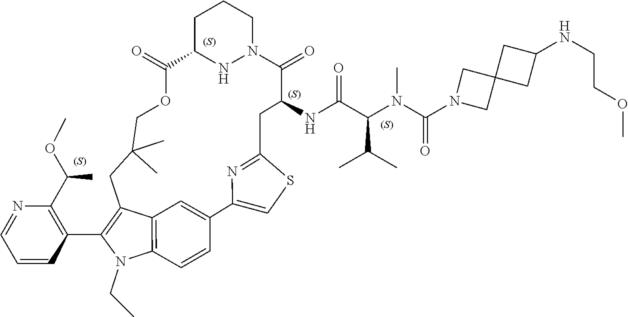 |
| AA513 | 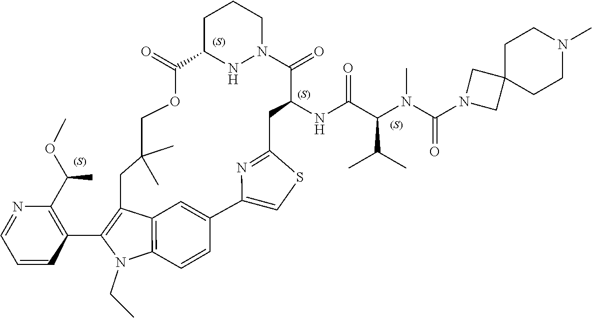 |
| AA514 | 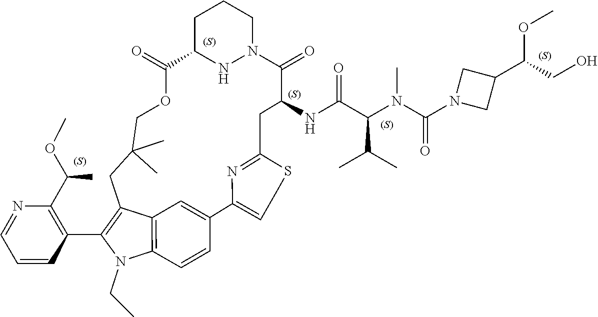 |
| AA515 | 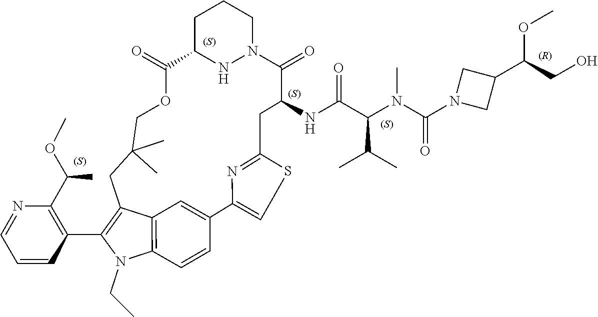 |
| AA516 | 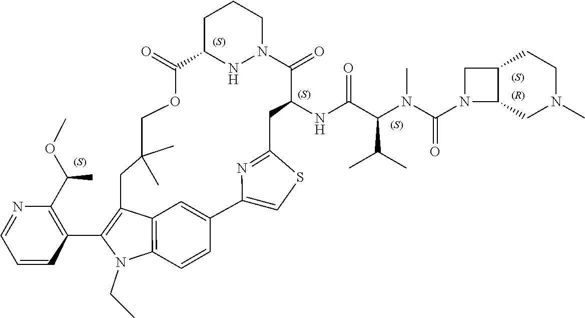 |
| AA517 | 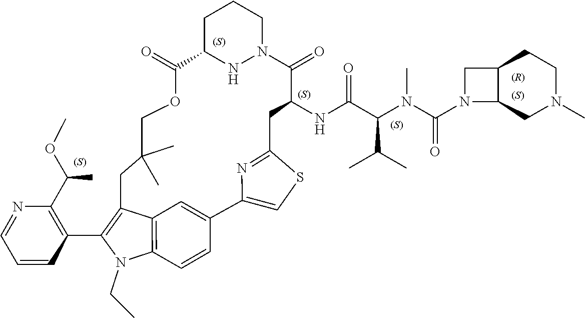 |
| AA518 | 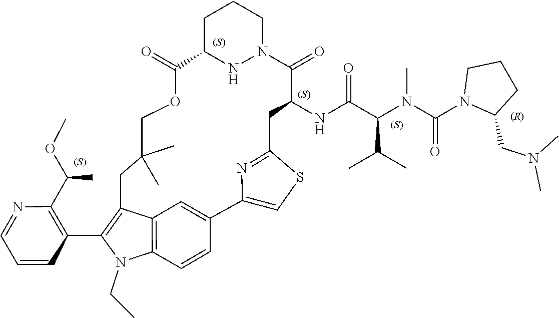 |
| AA519 | 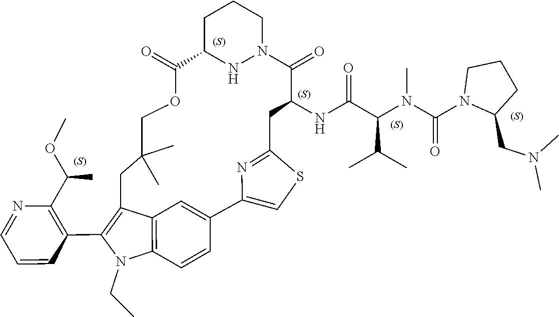 |
| AA520 | 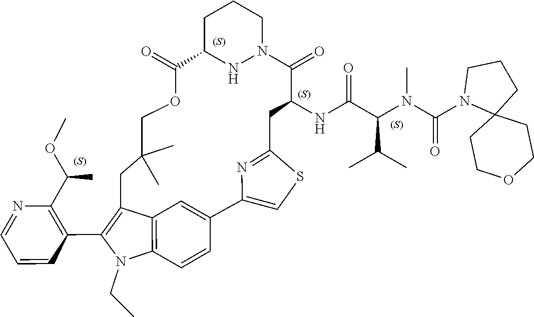 |
| AA521 | 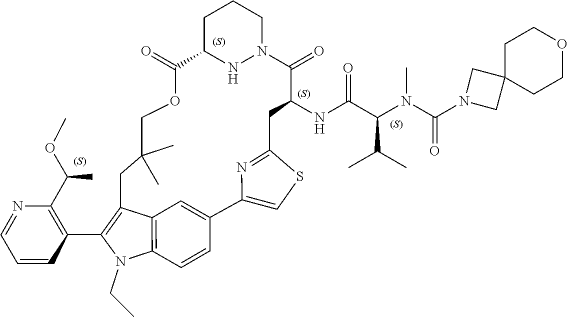 |
| AA522 | 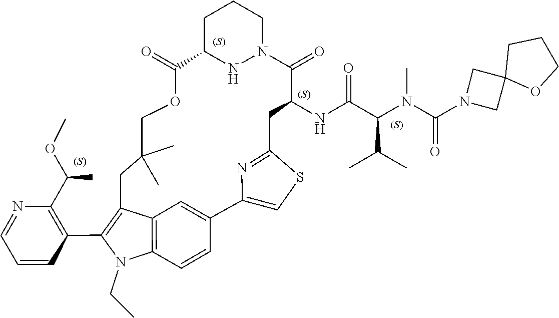 |
| AA523 | 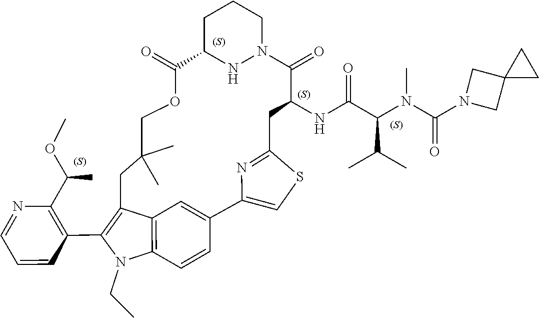 |
| AA524 | 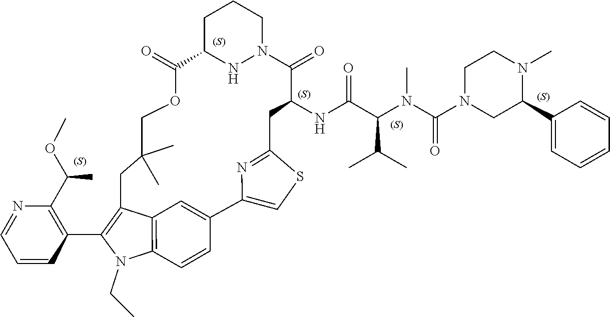 |
| AA525 | 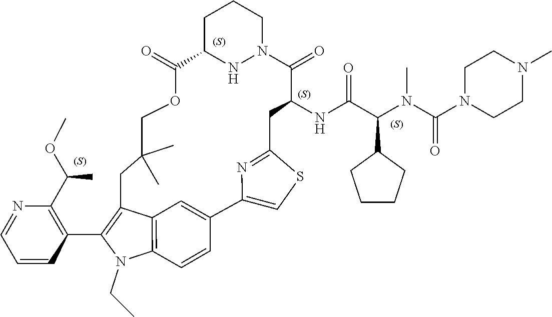 |
| AA526 | 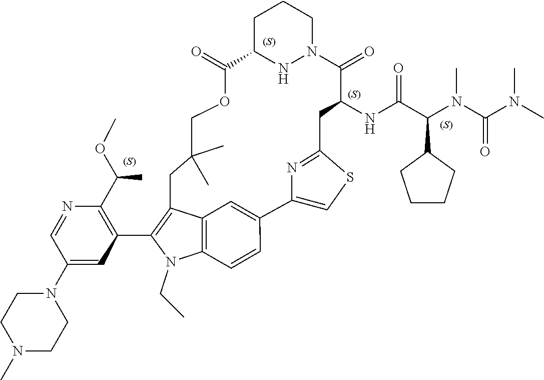 |
| AA527 | 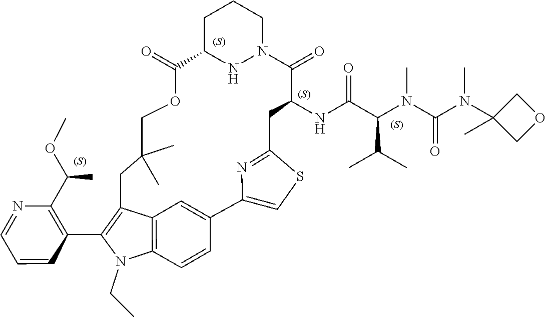 |
| AA528 | 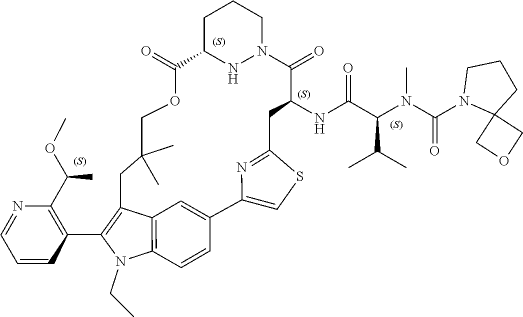 |
| AA529 | 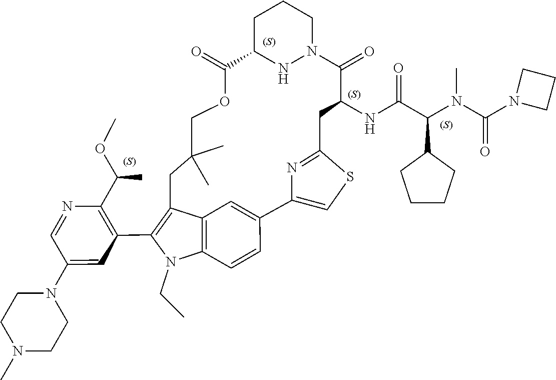 |
| AA530 | 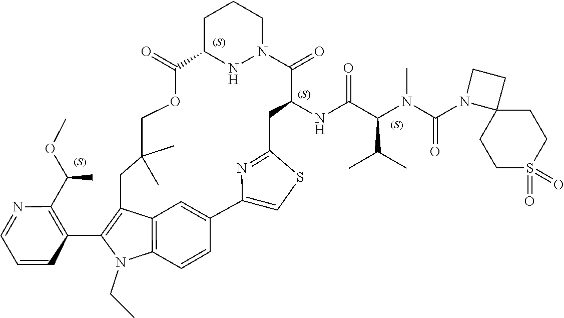 |
| AA531 | 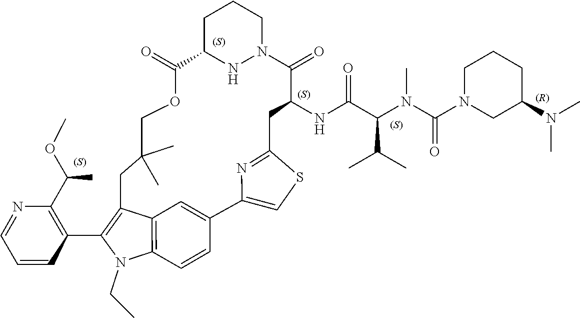 |
| AA532 | 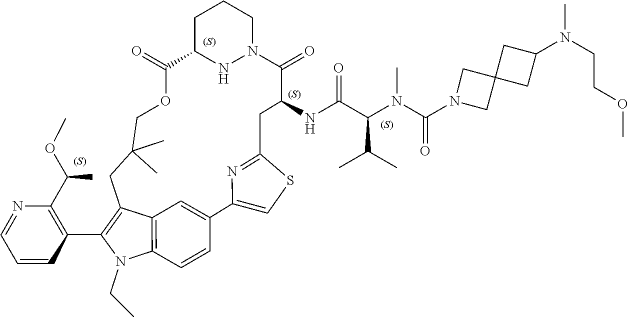 |
| AA533 | 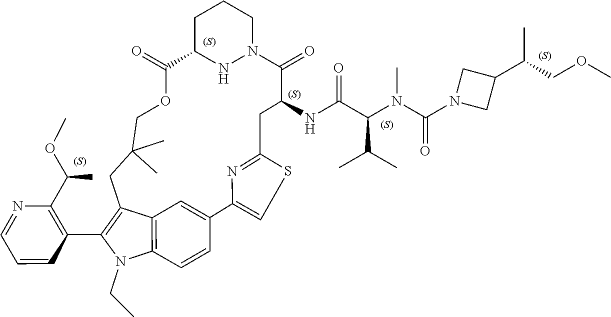 |
| AA534 | 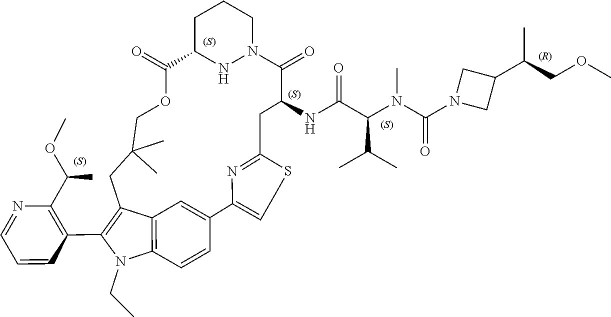 |
| AA535 | 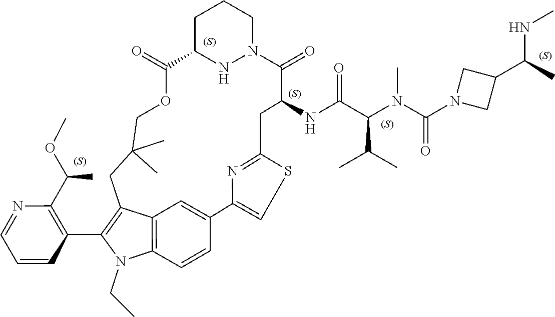 |
| AA536 | 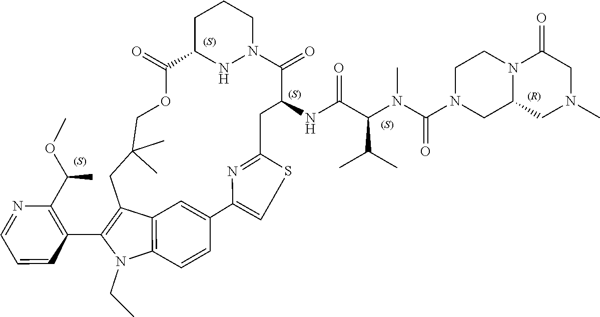 |
| AA537 | 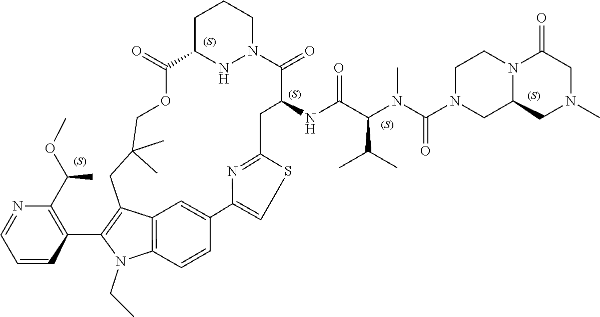 |
| AA538 | 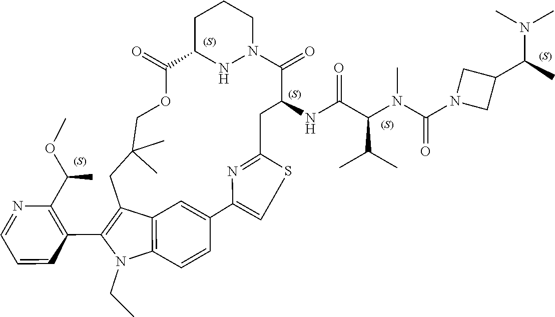 |
| AA539 | 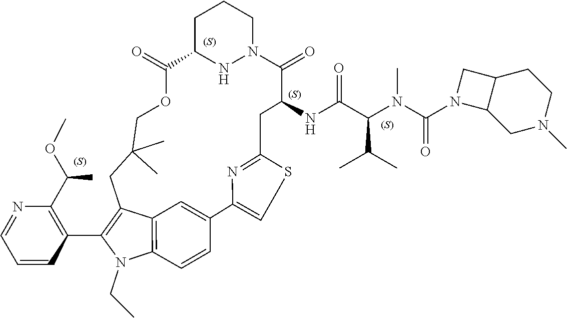 |
| AA540 | 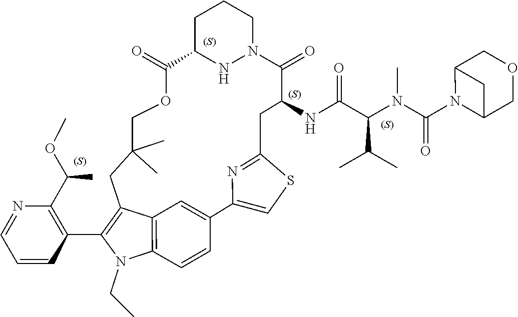 |
| AA541 | 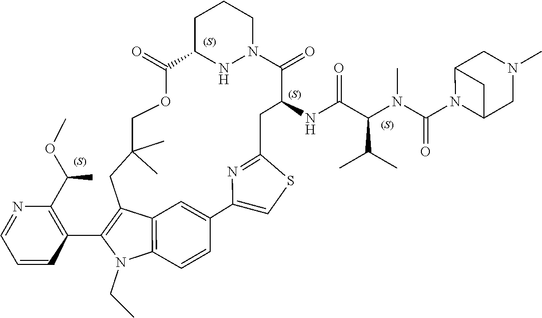 |
| AA542 | 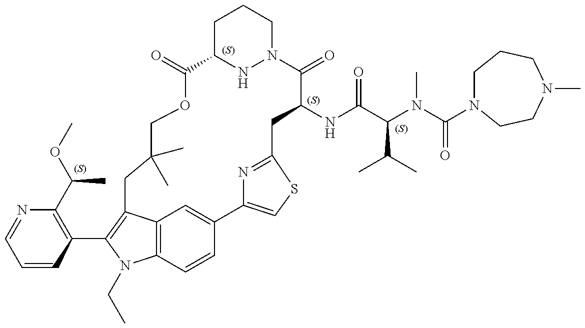 |
| AA543 | 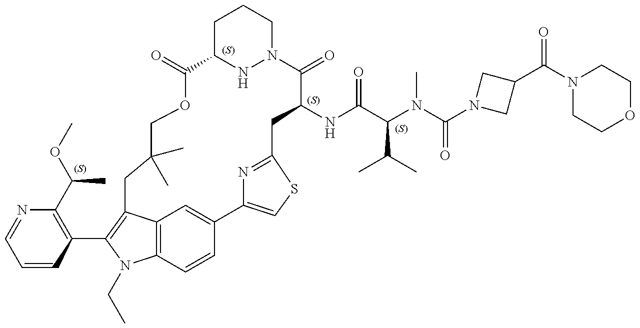 |
| AA544 | 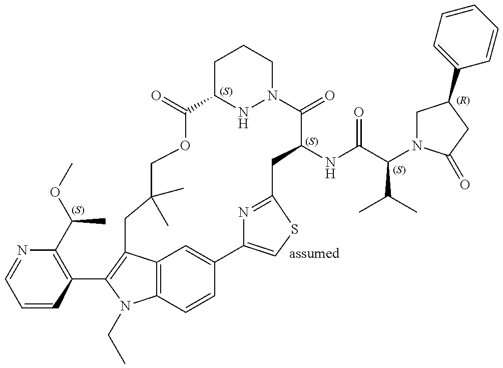 |
| AA545 | 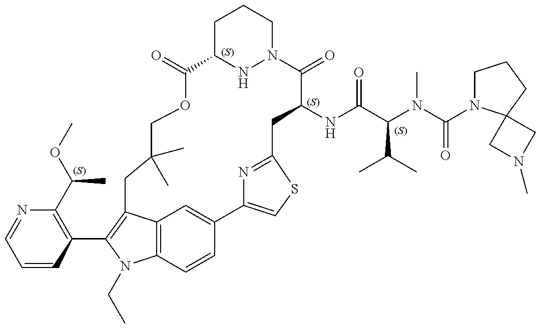 |
| AA546 | 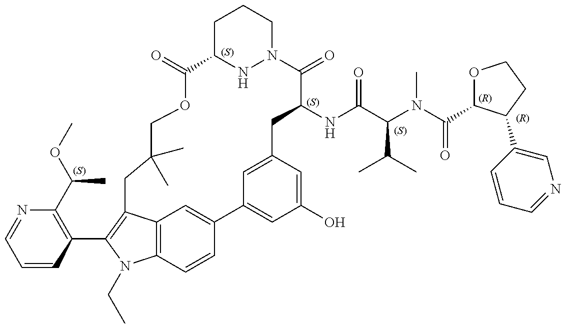 |
| AA547 | 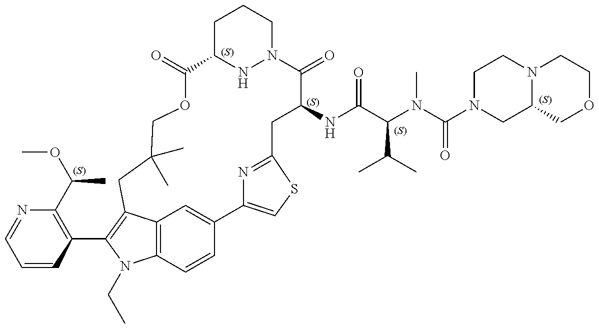 |
| AA548 | 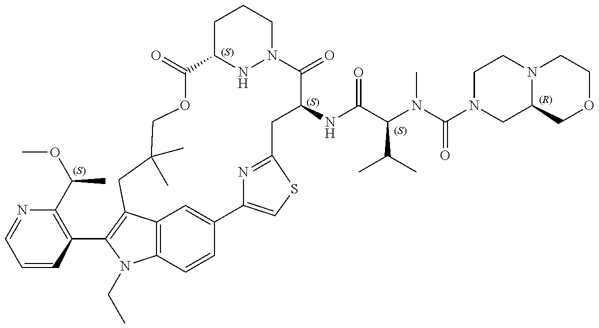 |
| AA549 | 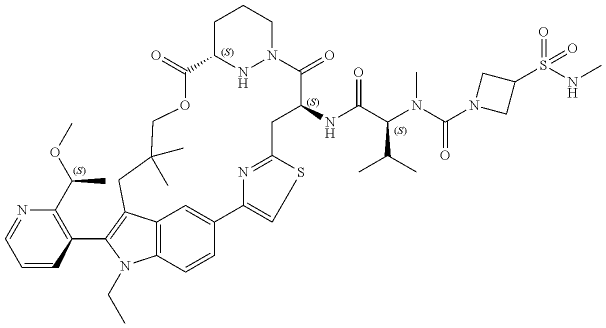 |
| AA550 | 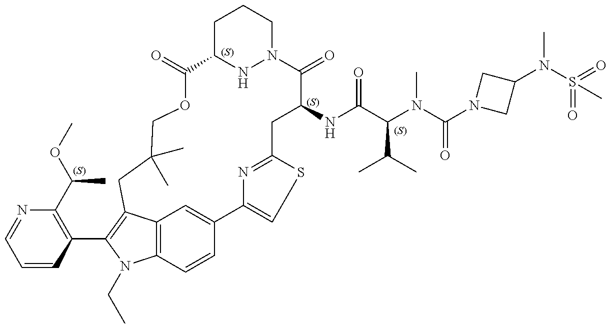 |
| AA551 | 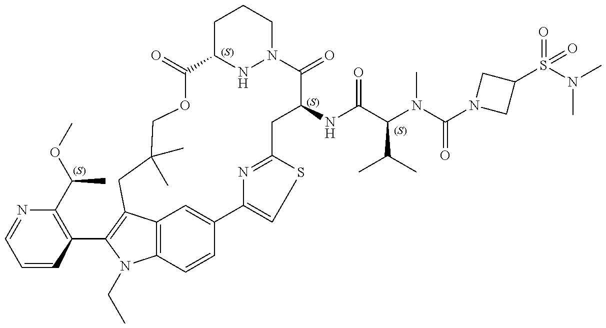 |
| AA552 | 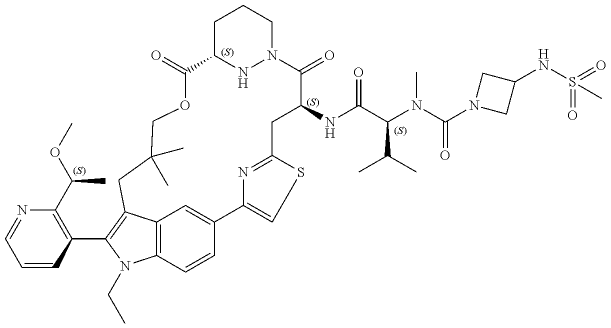 |
| AA553 | 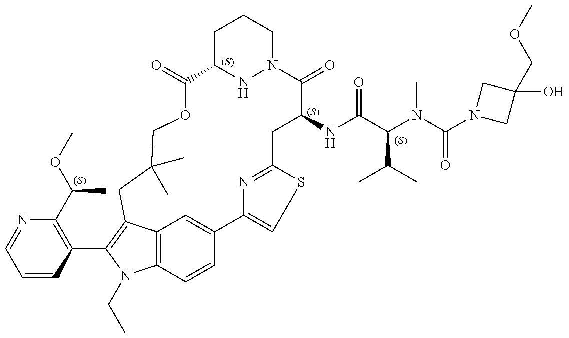 |
| AA554 | 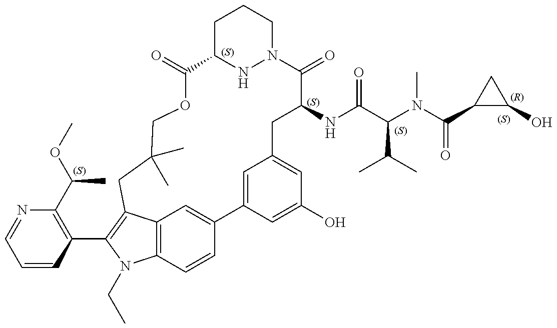 |
| AA555 | 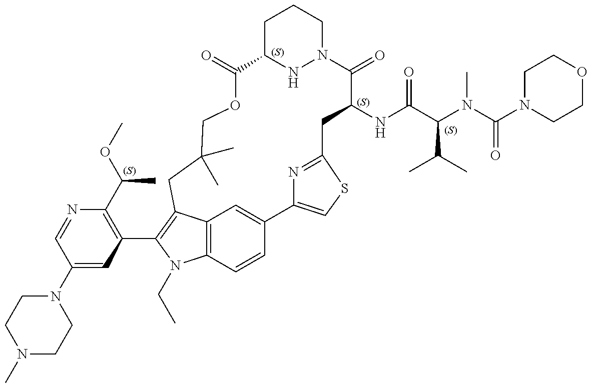 |
| AA556 | 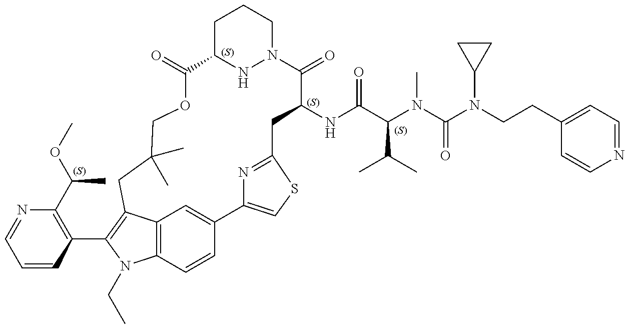 |
| AA557 | 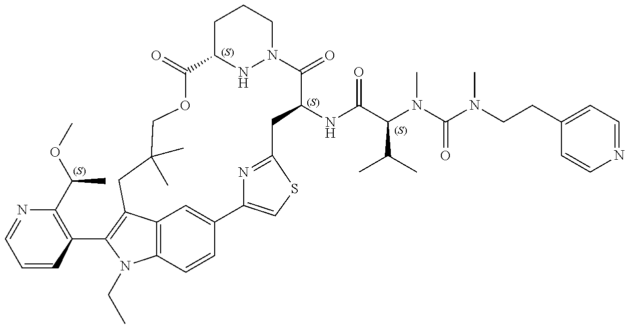 |
| AA558 | 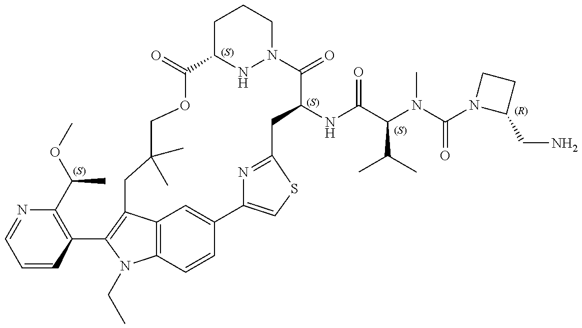 |
| AA559 | 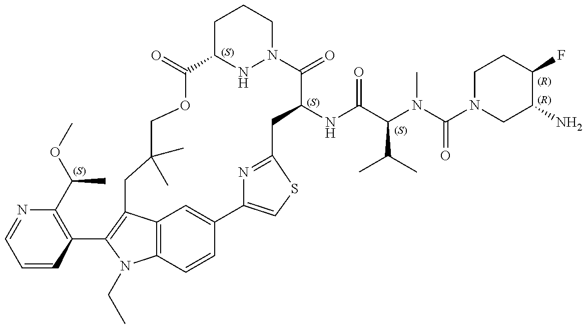 |
| AA560 | 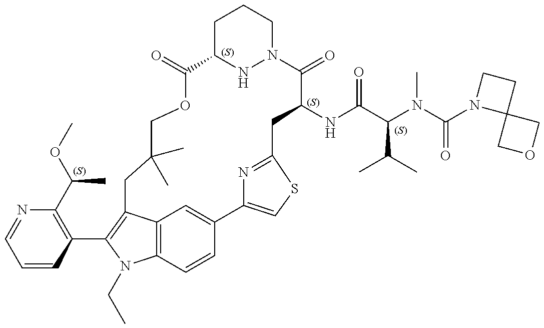 |
| AA561 | 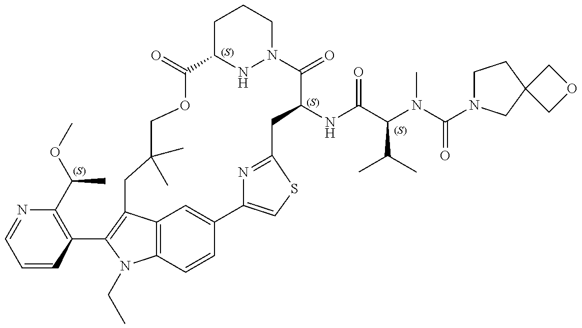 |
| AA562 | 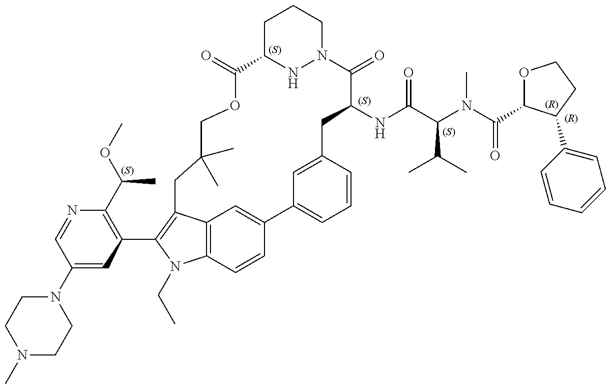 |
| AA563 | 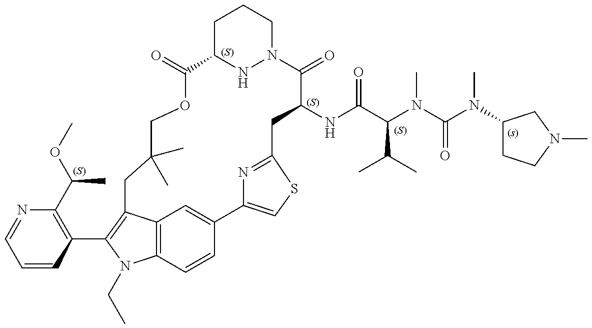 |
| AA564 | 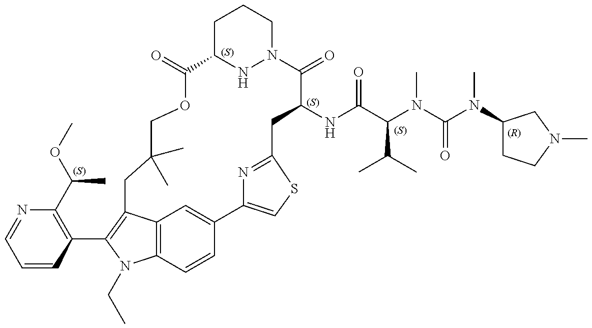 |
| AA565 | 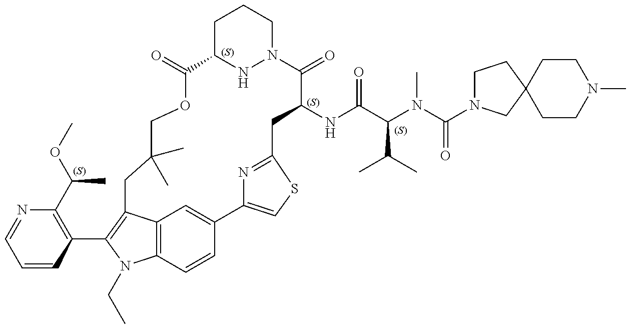 |
| AA566 | 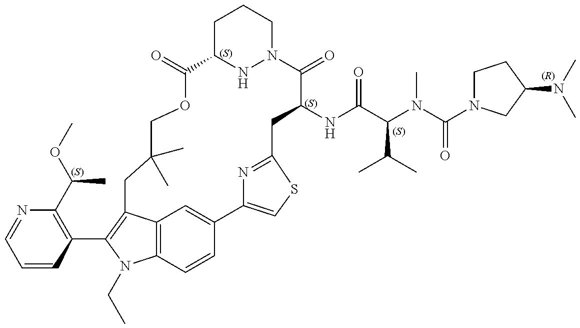 |
| AA567 | 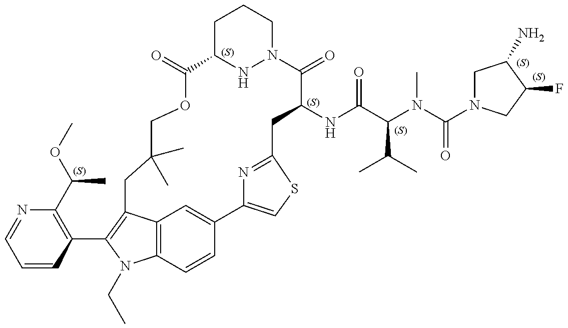 |
| AA568 | 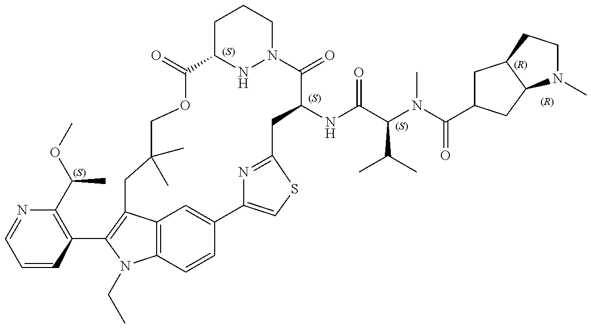 |
| AA569 | 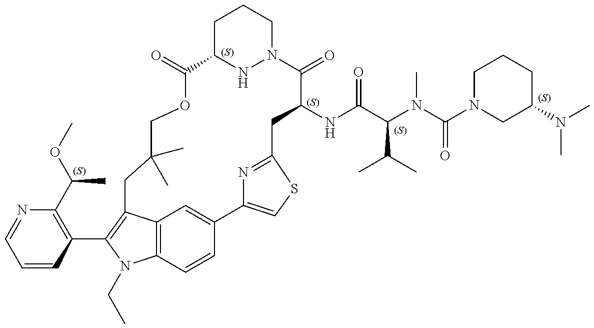 |
| AA570 | 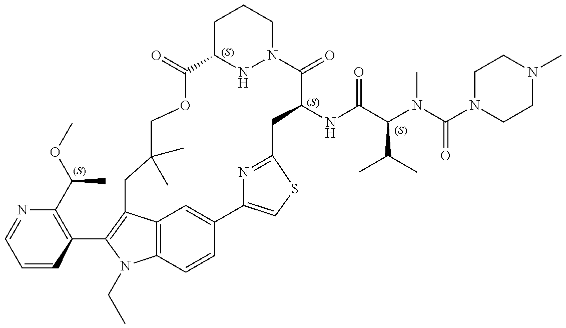 |
| AA571 | 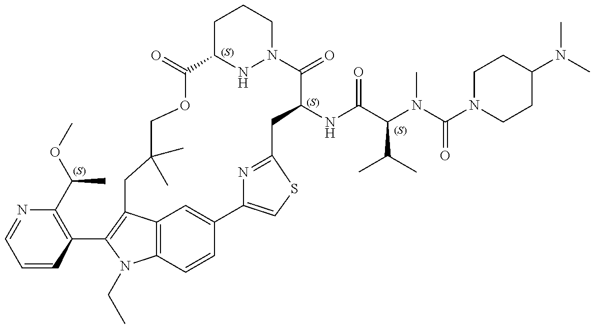 |
| AA572 | 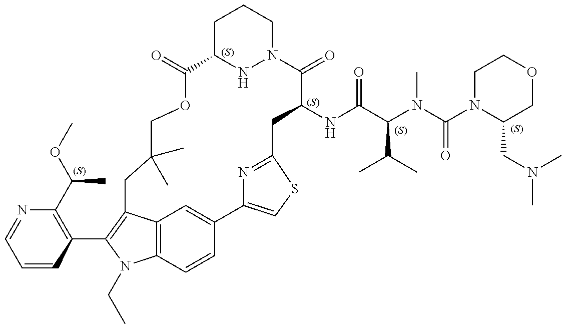 |
| AA573 | 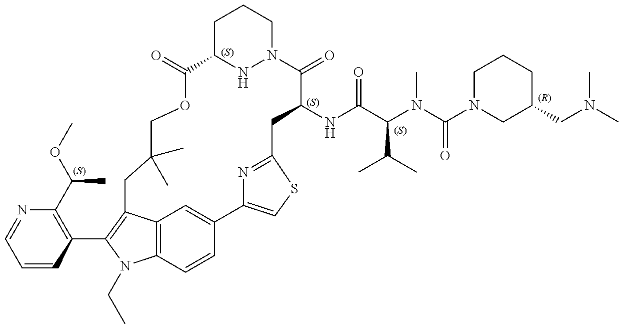 |
| AA574 | 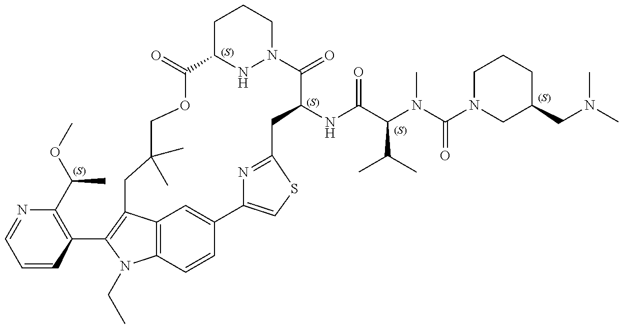 |
| AA575 | 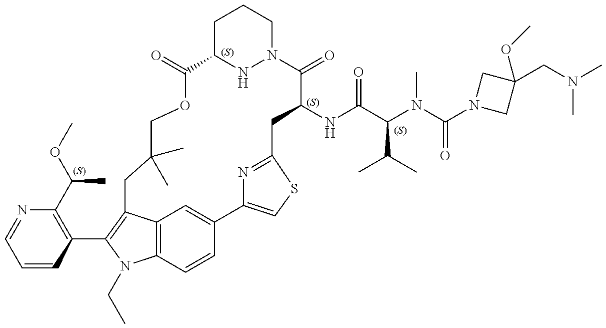 |
| AA576 | 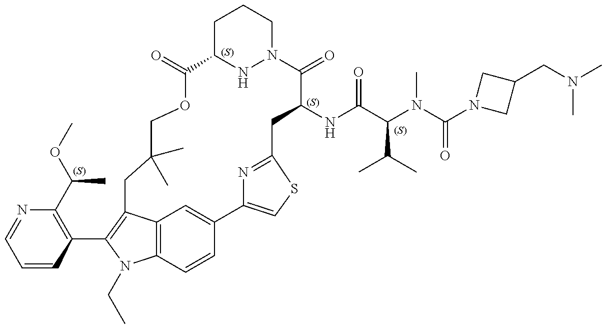 |
| AA577 | 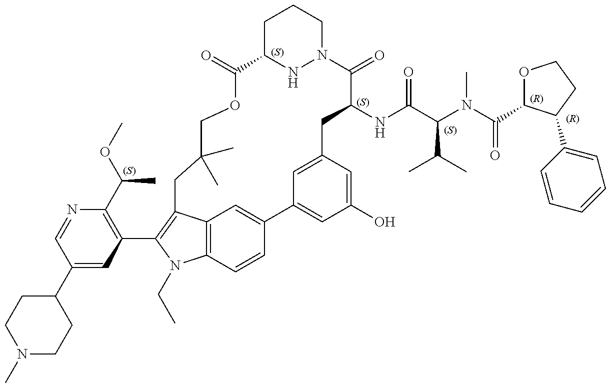 |
| AA578 | 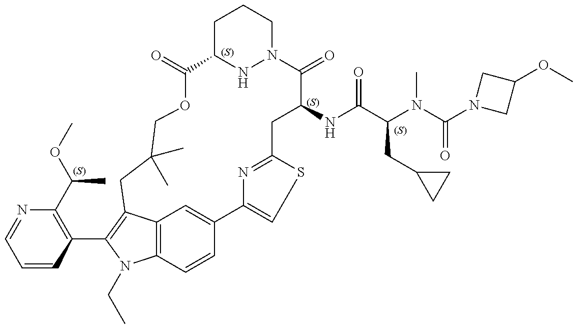 |
| AA579 | 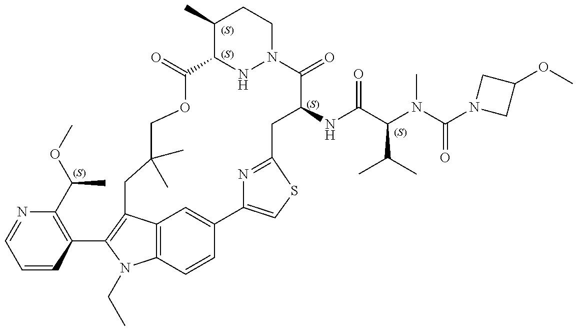 |
| AA580 | 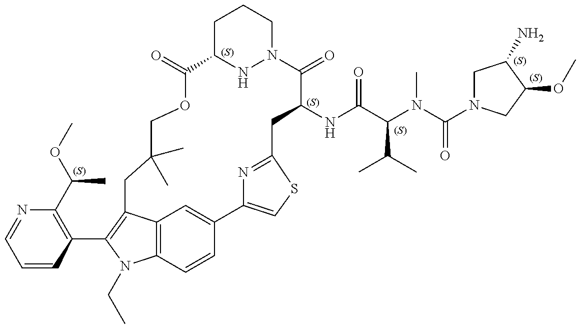 |
| AA581 | 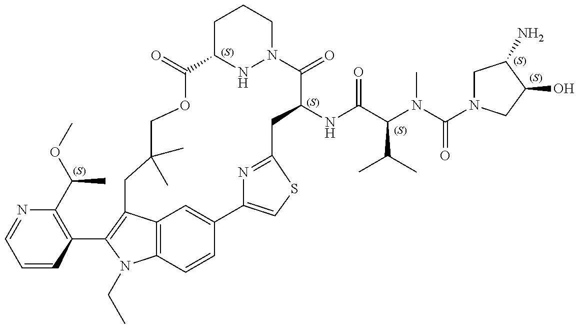 |
| AA582 | 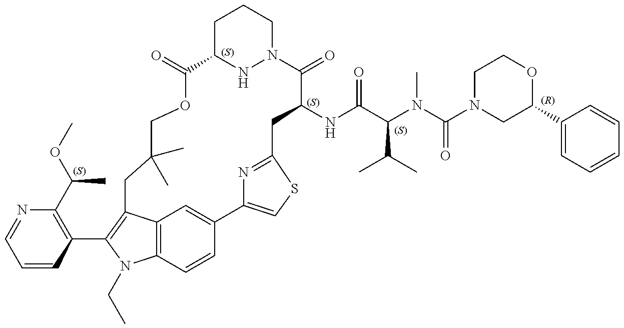 |
| AA583 | 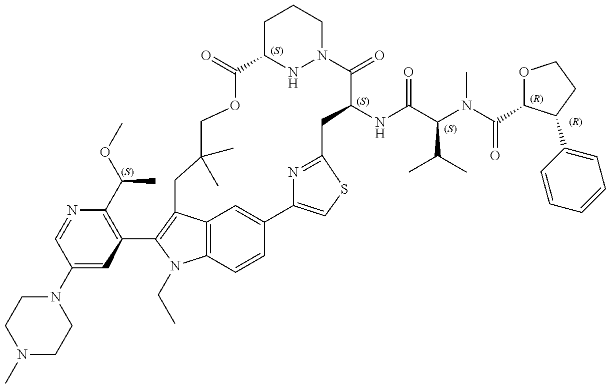 |
| AA584 | 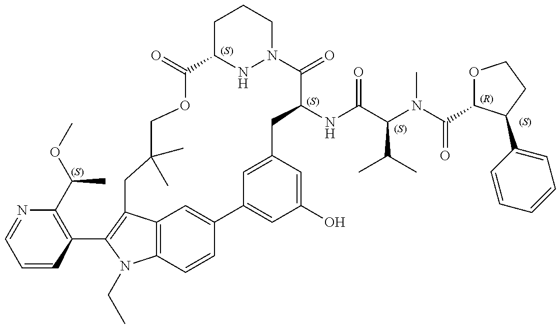 |
| AA585 | 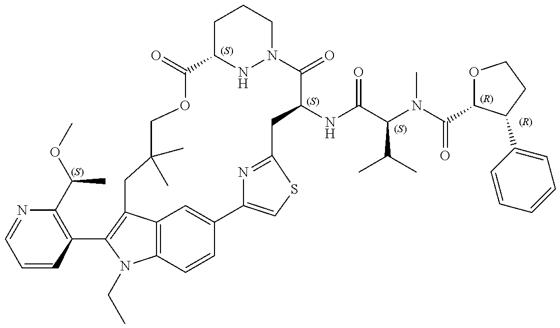 |
| AA586 | 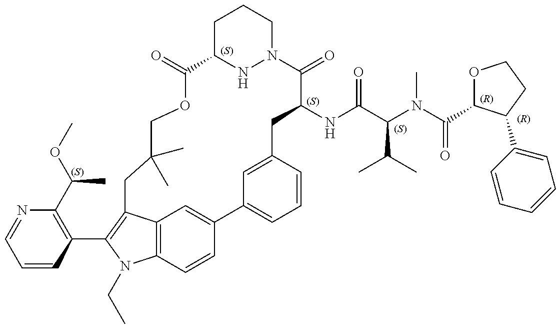 |
| AA587 | 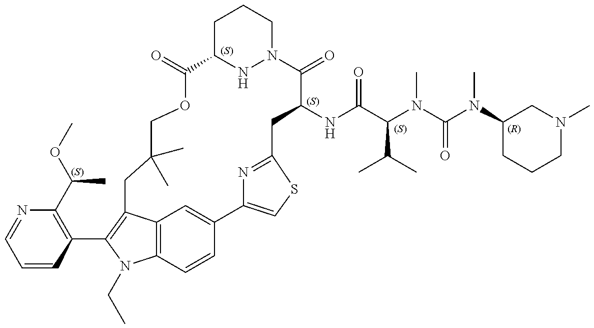 |
| AA588 | 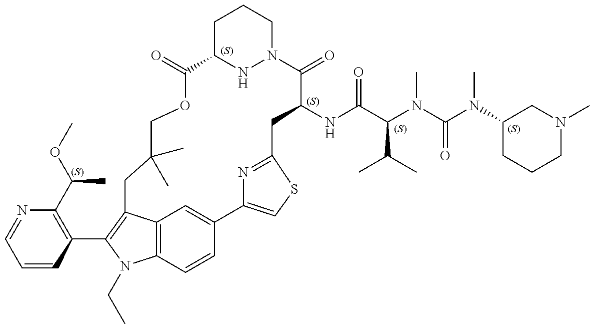 |
| AA589 | 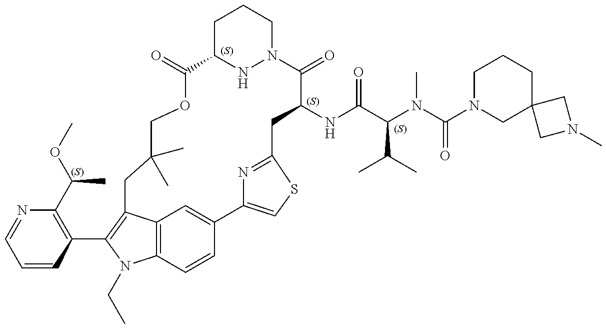 |
| AA590 | 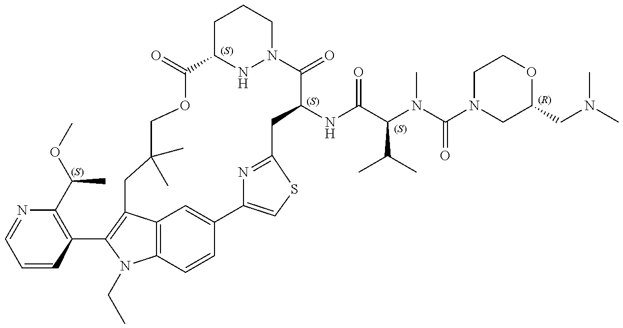 |
| AA591 | 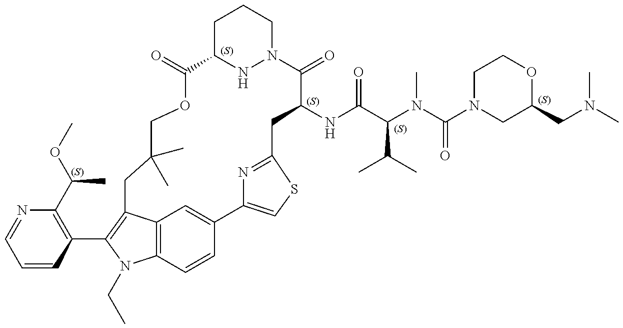 |
| AA592 | 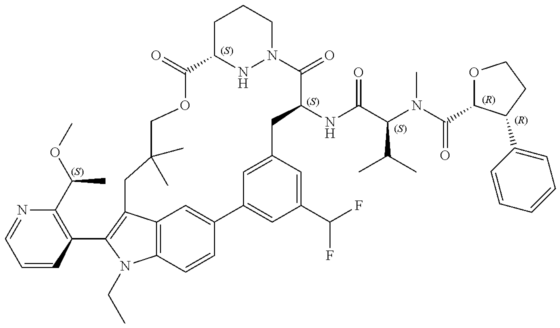 |
| AA593 | 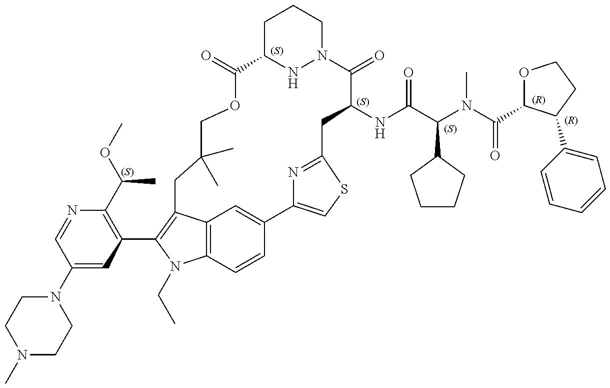 |
| AA594 | 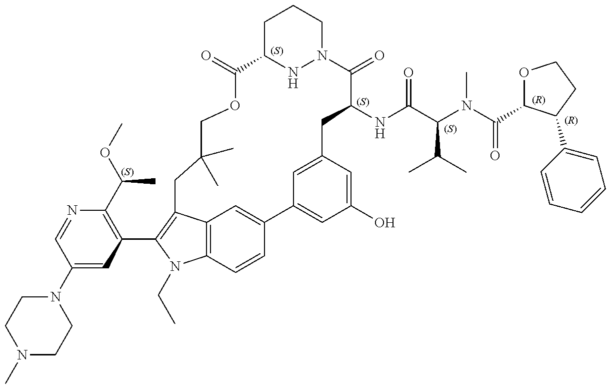 |
| AA595 | 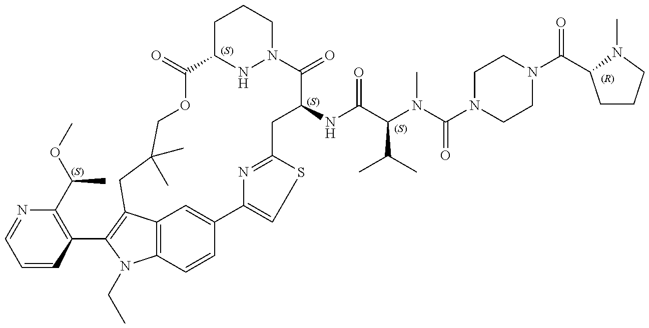 |
| AA596 | 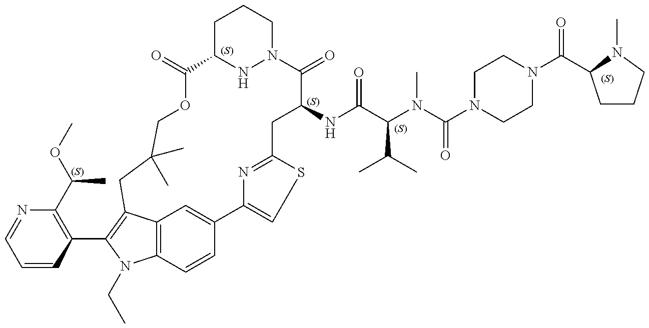 |
| AA597 | 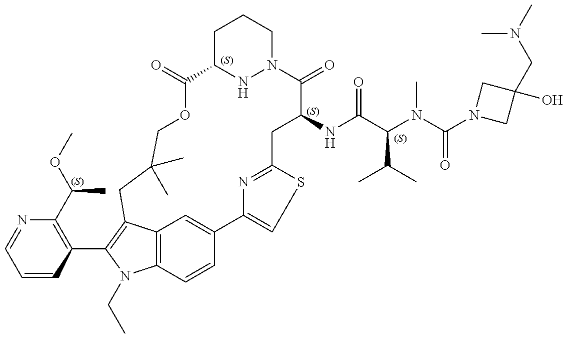 |
| AA598 | 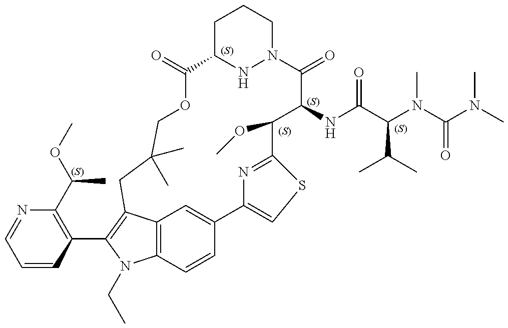 |
| AA599 | 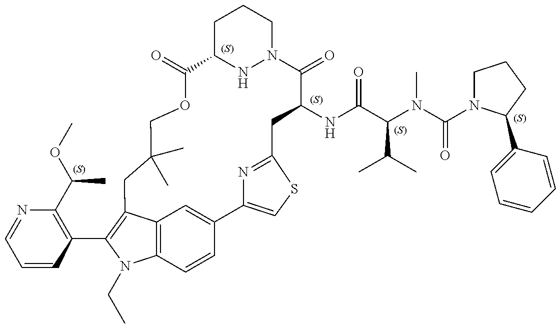 |
| AA600 | 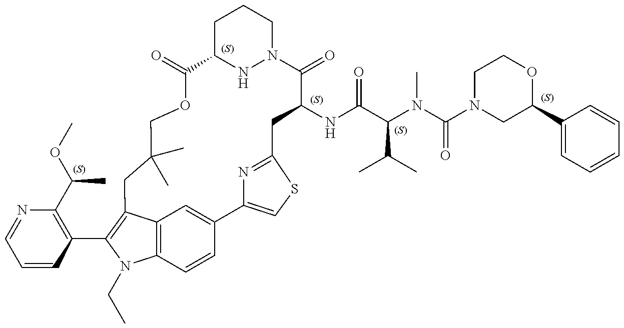 |
| AA601 | 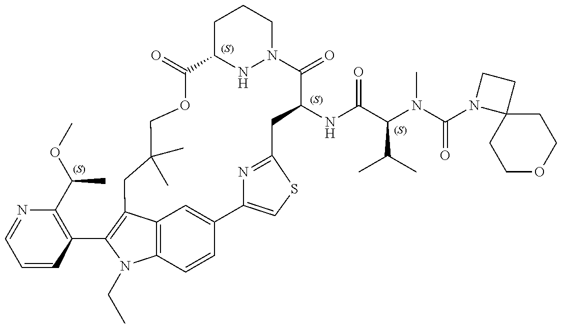 |
| AA602 | 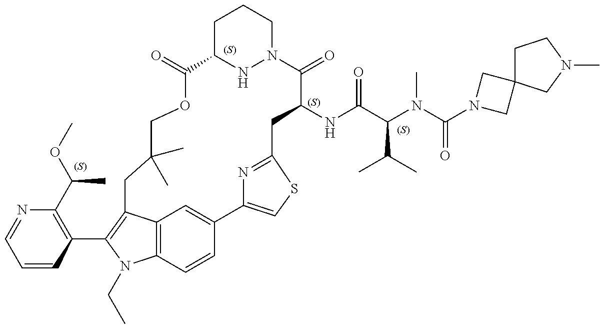 |
| AA603 | 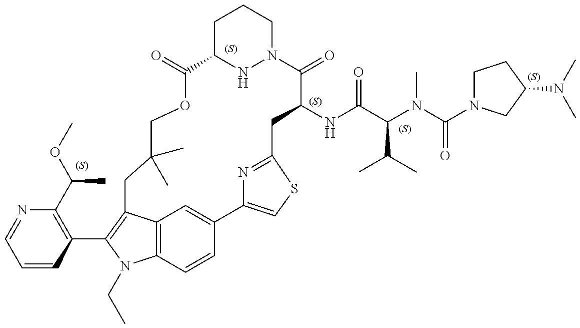 |
| AA604 | 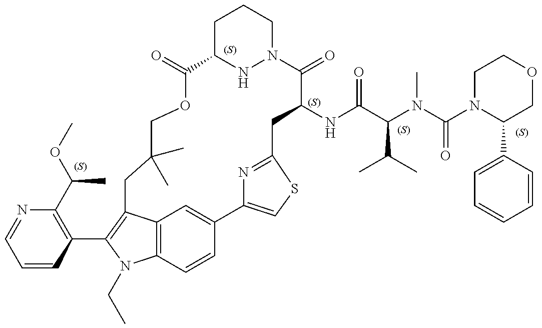 |
| AA605 | 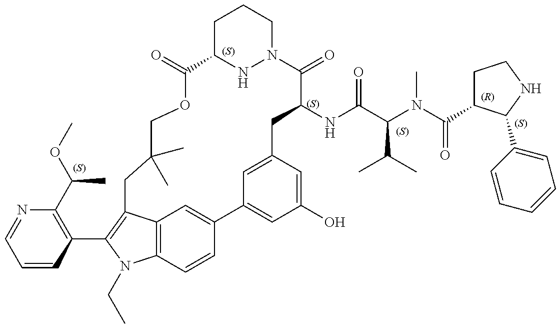 |
| AA606 | 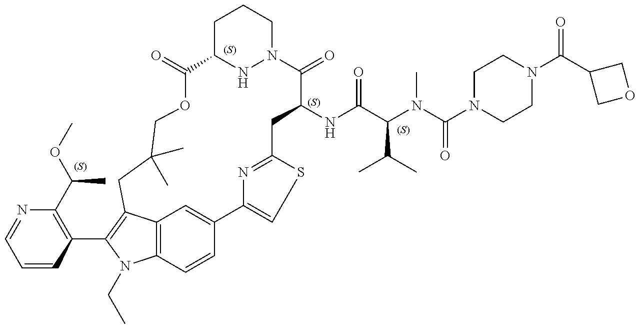 |
| AA607 | 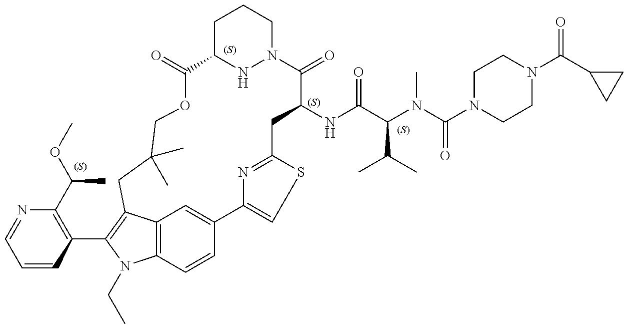 |
| AA608 | 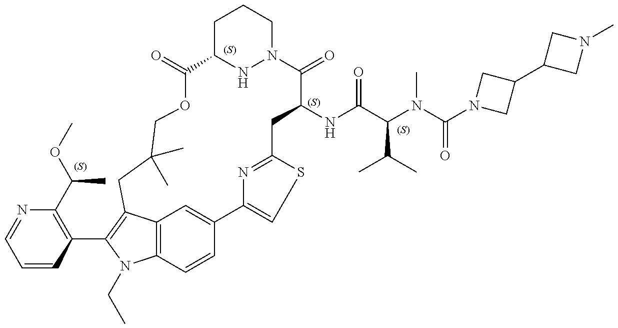 |
| AA609 | 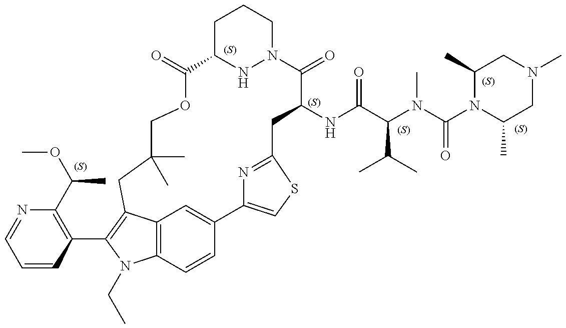 |
| AA610 | 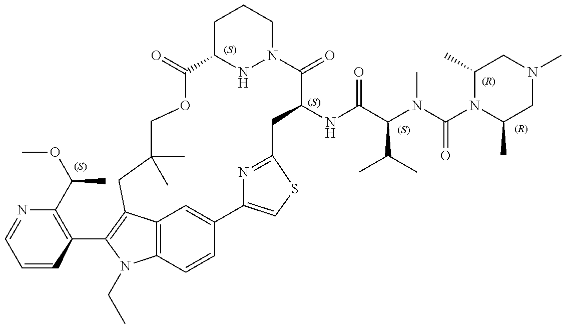 |
| AA611 | 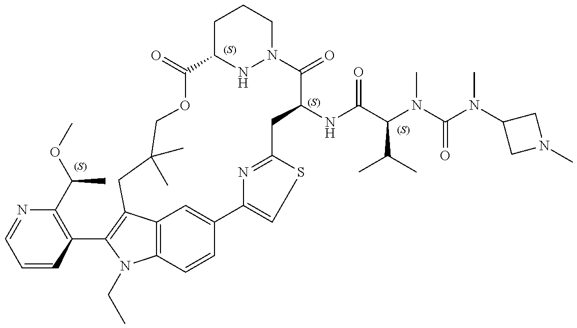 |
| AA612 | 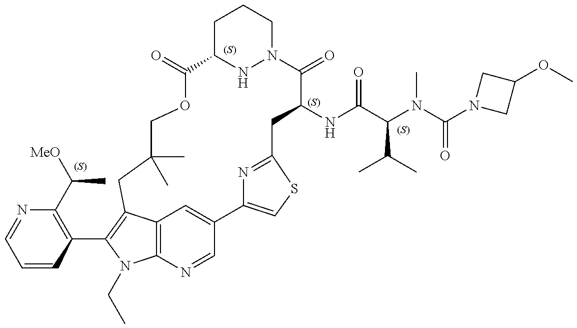 |
| AA613 | 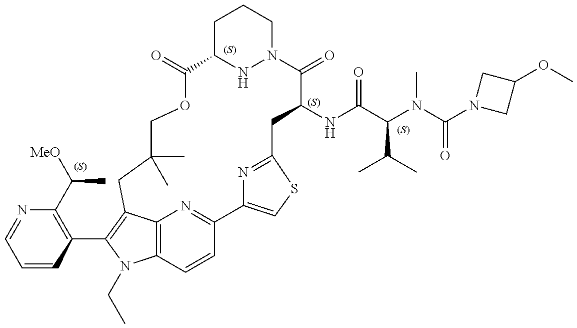 |
| AA614 | 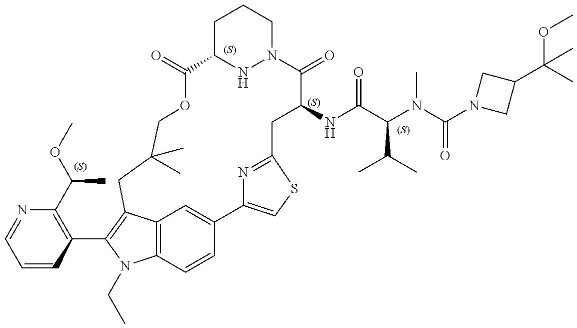 |
| AA615 | 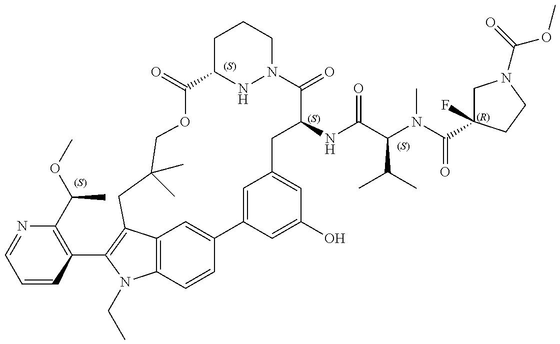 |
| AA616 | 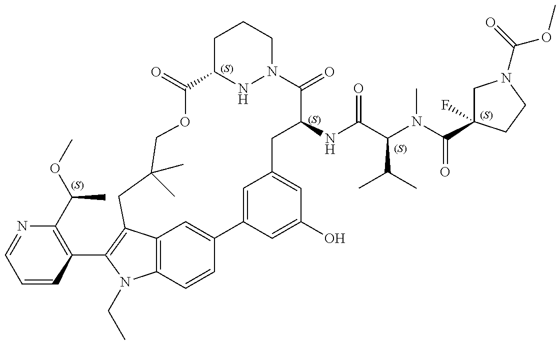 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contamplated by the present invention. In particular embodiments, an atropisomer of a compound of the foregoing table is contemplated. Any compound shown in brackets indicates that the compound is a disastereomer, and the absolute stereochemistry of such diastereomer may not be known.

In some embodiments, a compound of Table A2 is provided, or a pharmaceutically acceptable salt thereof. In some embodiments, the RAS(ON) inhibitor is selected from Table A2, or a pharmaceutically acceptable salt or atropisomer thereof.

TABLE A2
Certain Compounds of the Present Invention

| Ex # | Structure |
|---|---|
| AB4 | 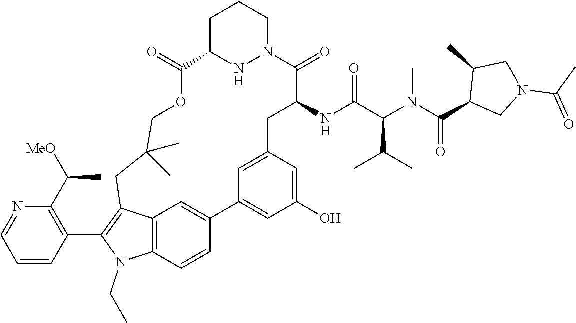 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB5 | 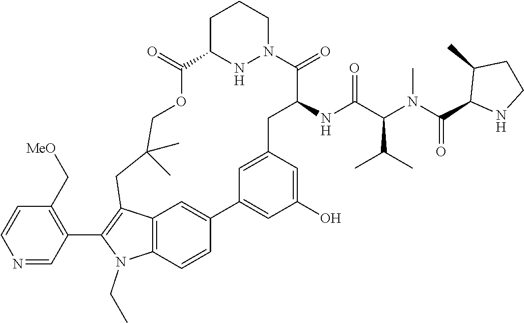 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB6 | 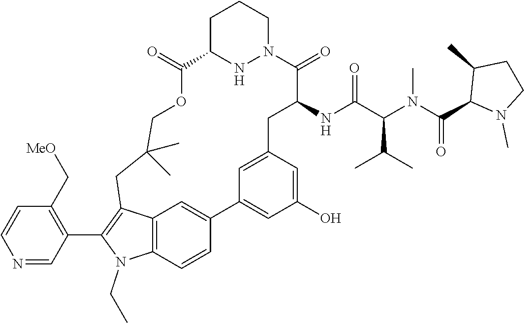 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB8 | 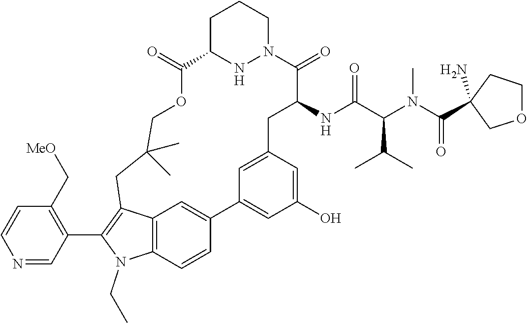 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB9 | 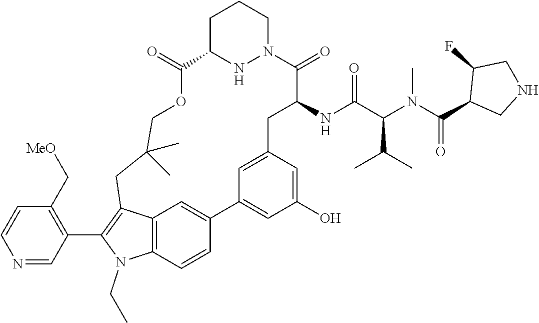 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB12 | 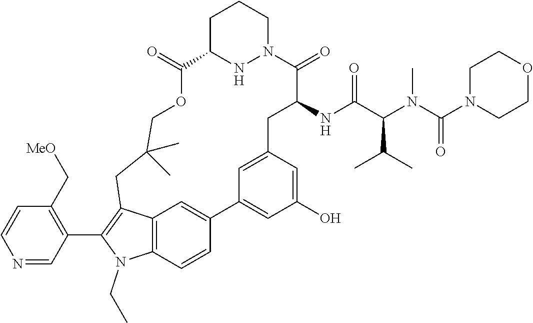 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AB13 | 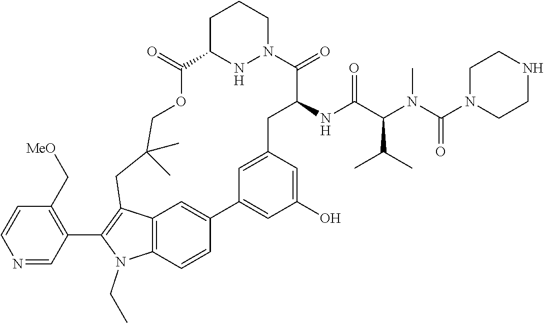 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AB19 | 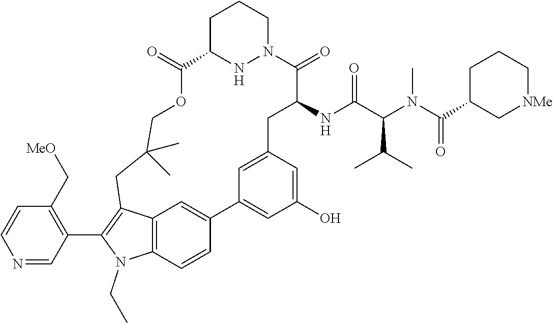 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| AB44 | 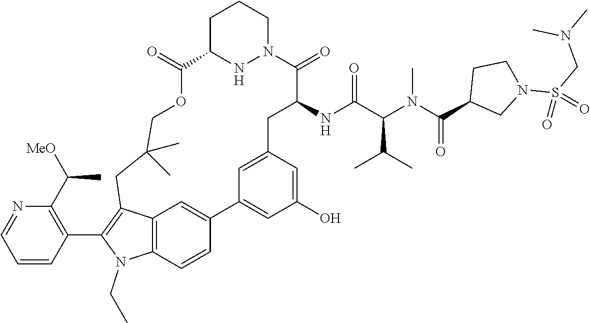 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)CN(C)C)C1)C(=O)N1CCC[C@H](N1)C(=O)OCC(C)(C)C2] |
| AB47 | 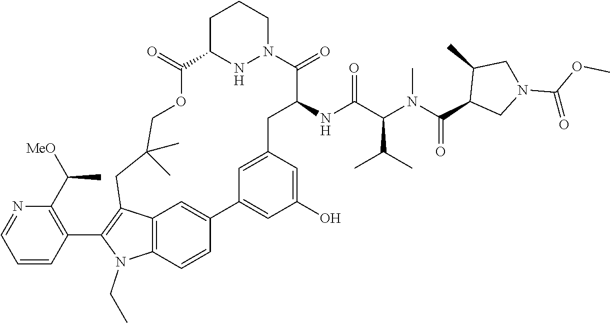 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2] |
| AB57 | 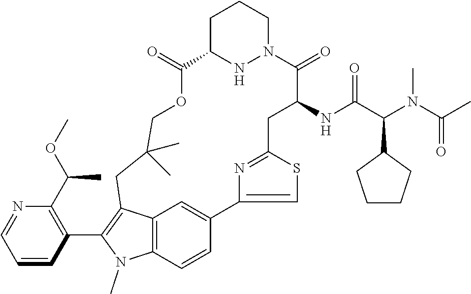 |
| AB58 | 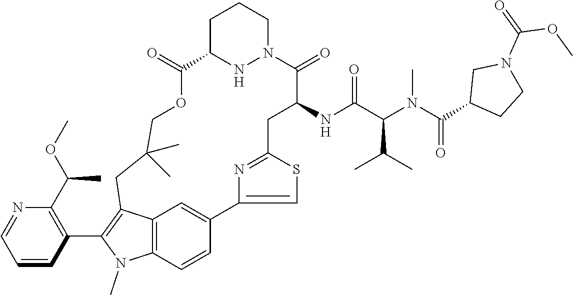 |
| AB59 | 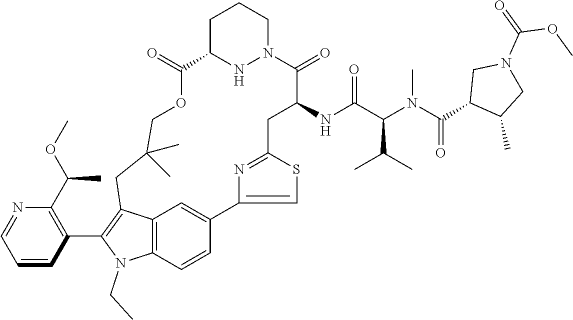 |
| AB60 | 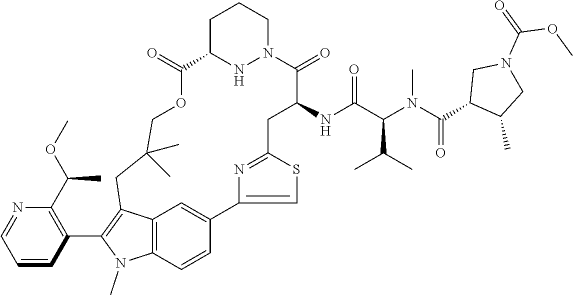 |
| AB61 | 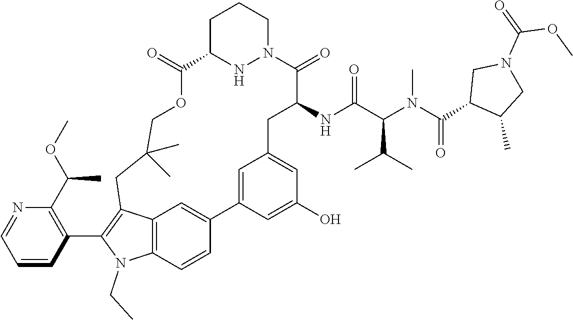 |
| AB66 | 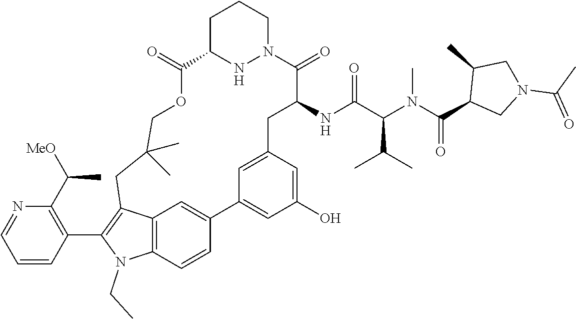 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB67 | 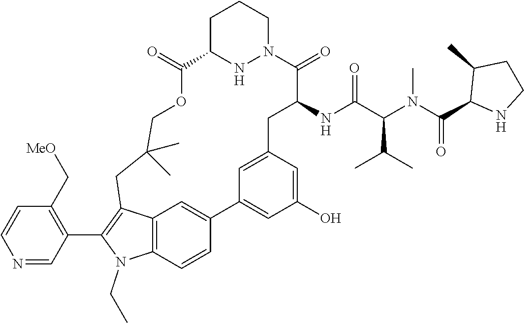 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB69 | 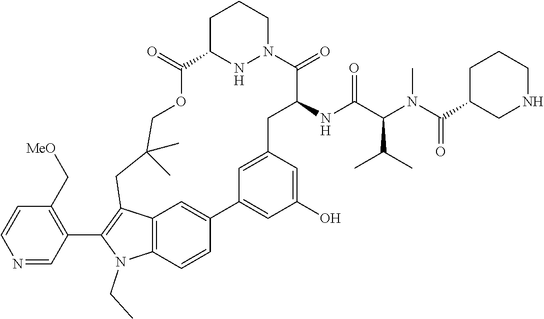 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCNC1)C(=O)N1CCC[C@H](N1)C(=O)OCC(C)(C)C2] |
| AB71 | 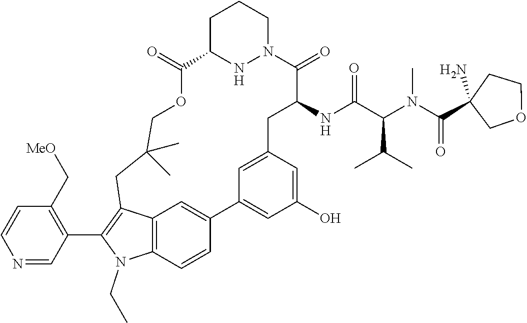 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB73 | 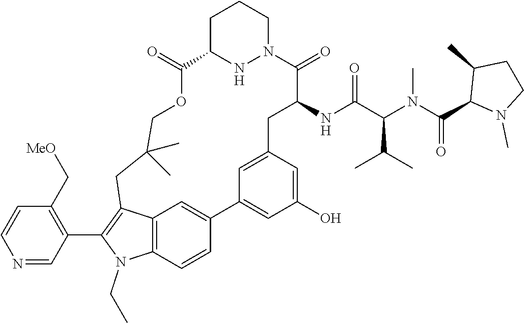 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB74 | 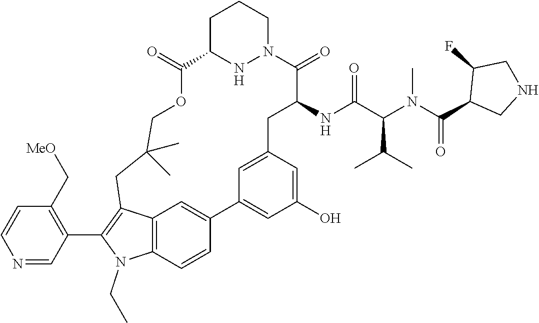 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB80 | 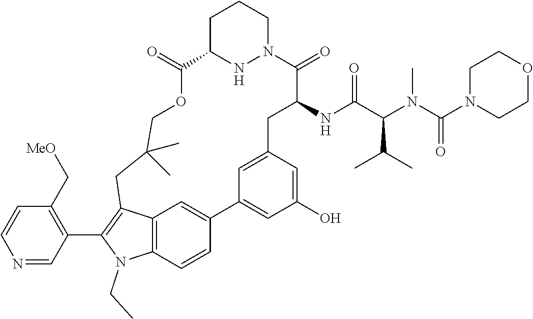 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AB81 | 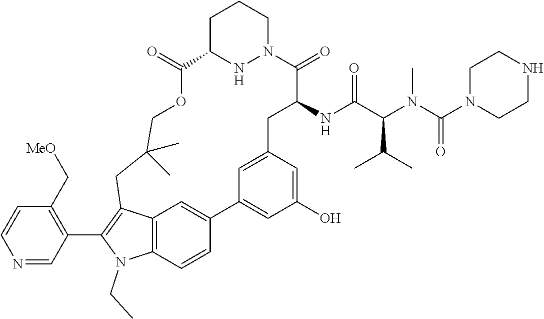 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AB94 | 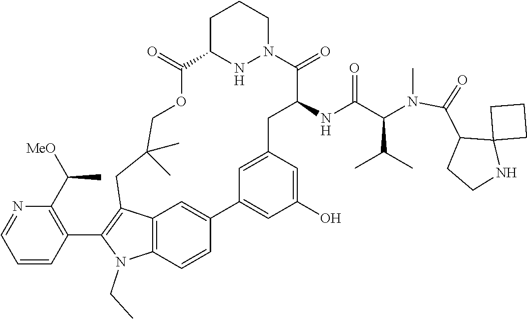 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CCC3)C(=O)N1CCC[C@H](N1)C(=O)OCC(C)(C)C2] |
| AB95 | 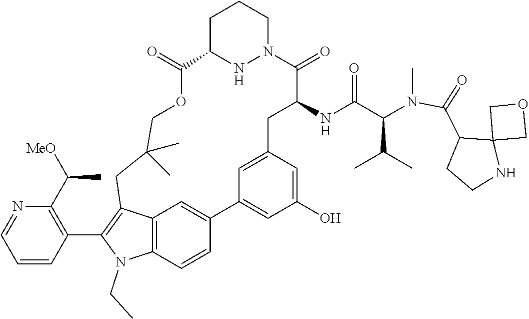 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13COC3)C(=O)N1CCC[C@H](N1)C(=O)OCC(C)(C)C2] |
| AB96 | 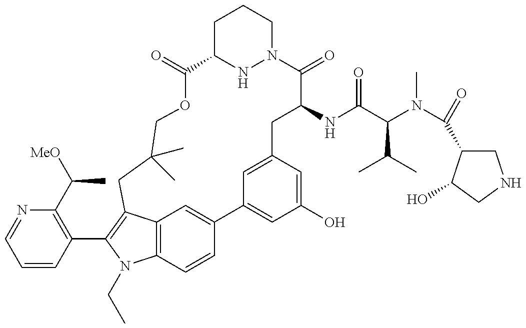 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB97 | 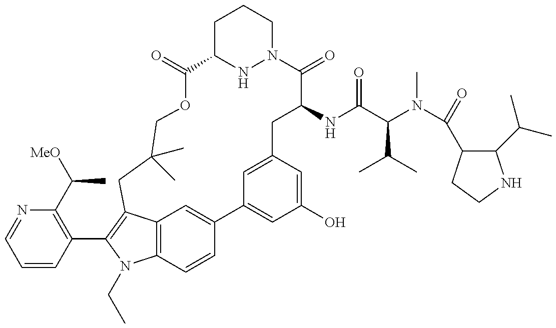 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC1C(C)C)C(=O)N1CCC[C@H](N1)C(=O)OCC(C)(C)C2] |
| AB99 | 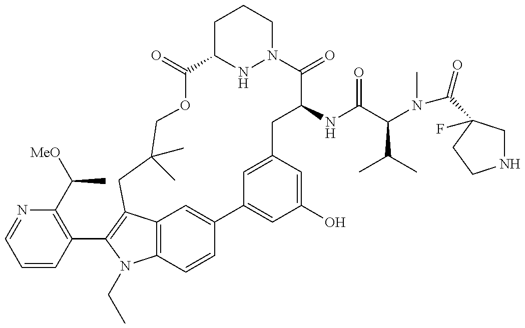 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(F)CCNC1)C(=O)N1CCC[C@H](N1)C(=O)OCC(C)(C)C2] |
| AB100 | 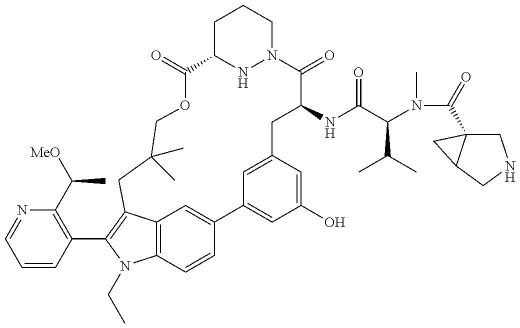 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@]13CNCC1C3)C(=O)N1CCC[C@H](N1)C(=O)OCC(C)(C)C2] |
| AB104 | 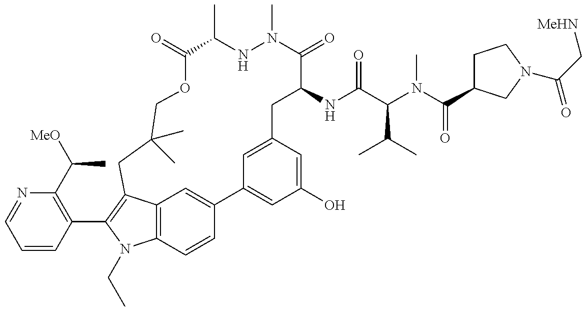 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N(C)N[C@@H](C)C(=O)OCC(C)(C)C2] |
| AB106 | 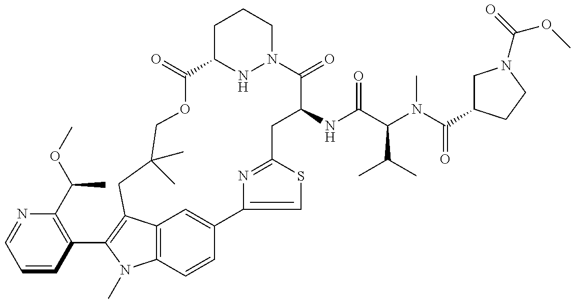 |
| AB107 | 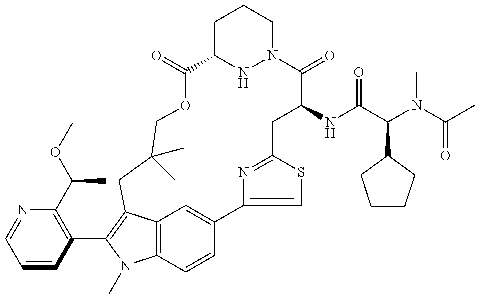 |
| AB109 | 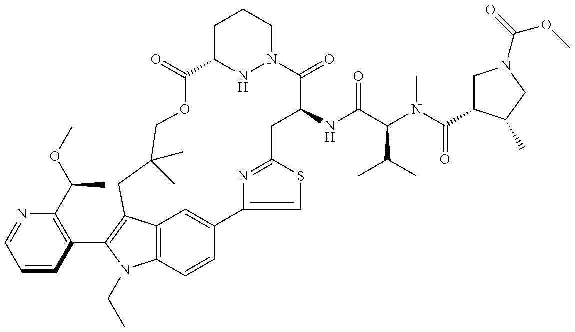 |
| AB110 | 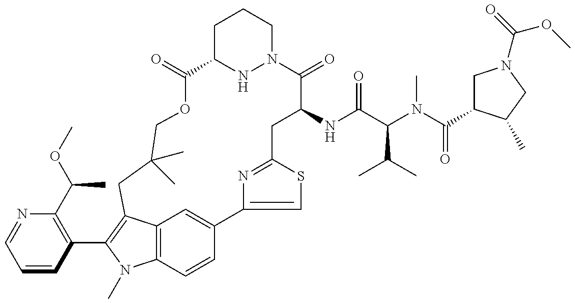 |
| AB111 | 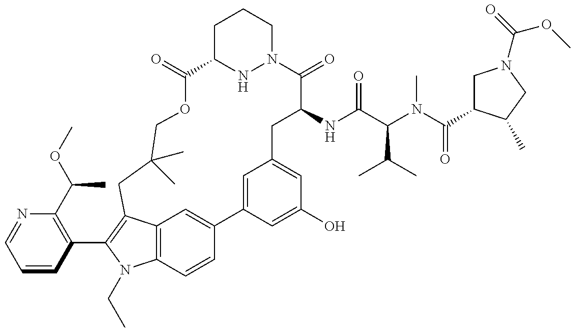 |
| AB112 | 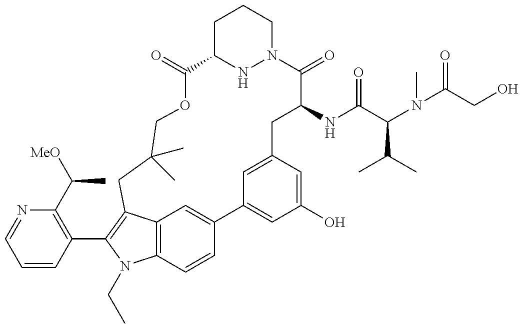 [SMILES: CCn1c(-c2cccnc2[C@H](C)OC)c2c3cc(ccc31)-c1cc(O)cc(c1)C[C@H](NC(=O)[C@H](C(C)C)N(C)C(=O)CO)C(=O)N1CCC[C@H](N1)C(=O)OCC(C)(C)C2] |
| AB113 | 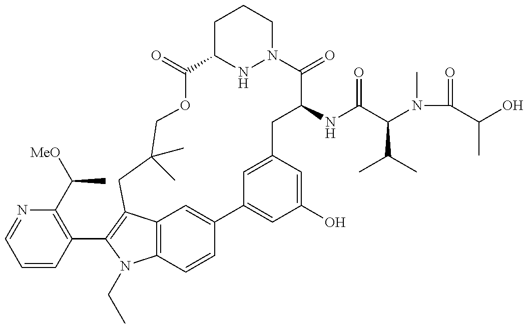 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(C)O)C(=O)N1CCC[C@H](N1)C(=O)OCC(C)(C)C2] |
| AB114 | 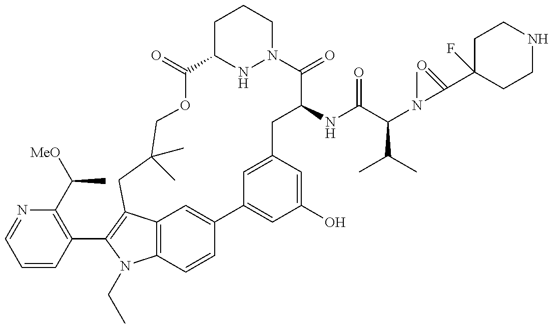 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(F)CCNCC1)C(=O)N1CCC[C@H](N1)C(=O)OCC(C)(C)C2] |
| AB117 | 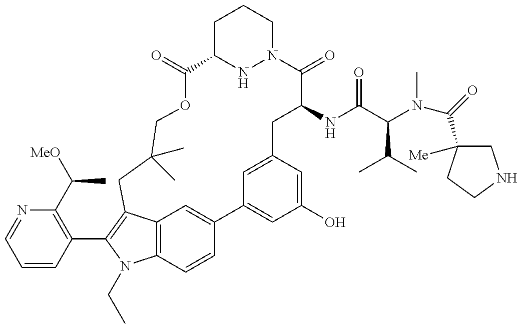 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCNC1)C(=O)N1CCC[C@H](N1)C(=O)OCC(C)(C)C2] |
| AB119 | 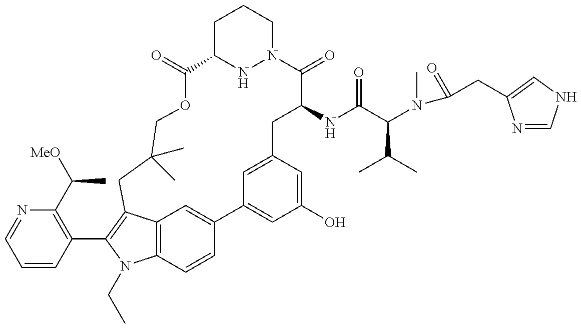 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1c[nH]cn1)C(=O)N1CCC[C@H](N1)C(=O)OCC(C)(C)C2] |
| AB122 | 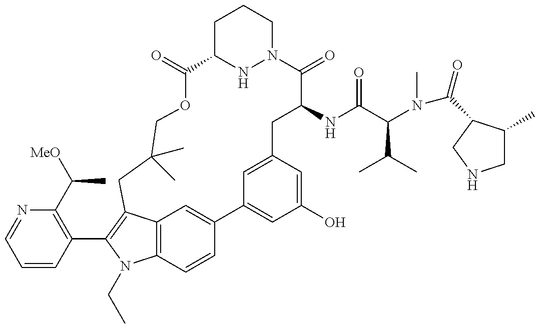 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CNC[C@H]1C)C(=O)N1CCC[C@H](N1)C(=O)OCC(C)(C)C2] |
| AB123 | 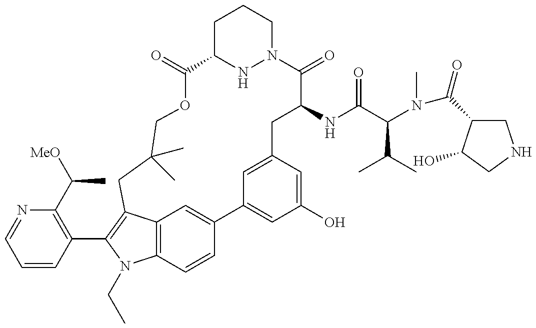 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB124 | 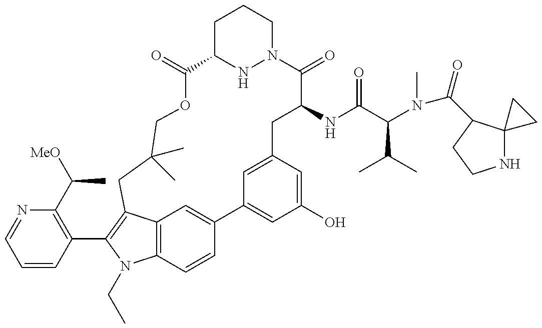 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CC3)C(=O)N1CCC[C@H](N1)C(=O)OCC(C)(C)C2] |
| AB126 | 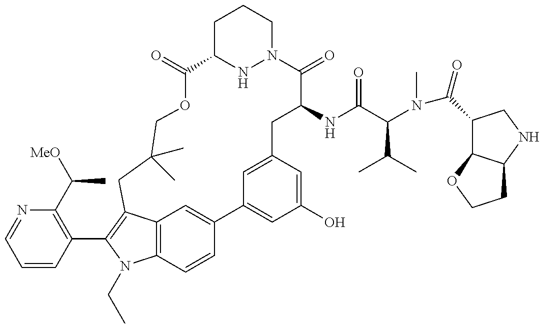 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN[C@H]3CCO[C@H]31)C(=O)N1CCC[C@H](N1)C(=O)OCC(C)(C)C2] |
| AB128 | 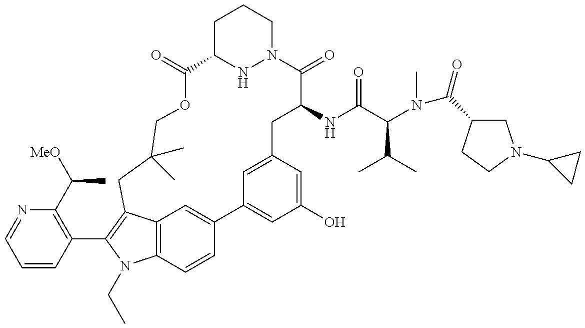 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C3CC3)C1)C(=O)N1CCC[C@H](N1)C(=O)OCC(C)(C)C2] |
| AB129 | 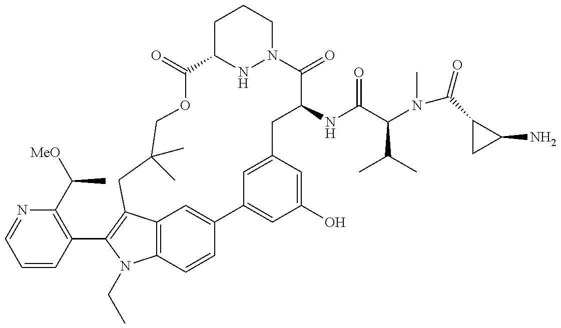 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H]1N)C(=O)N1CCC[C@H](N1)C(=O)OCC(C)(C)C2] |
| AB130 | 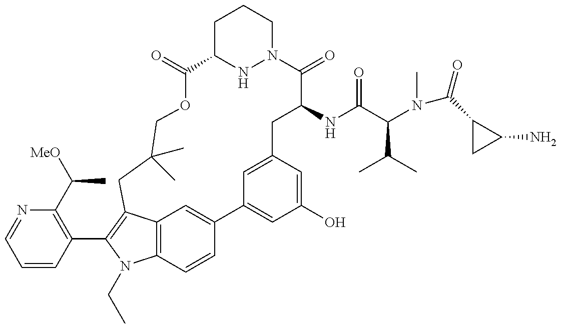 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@H]1N)C(=O)N1CCC[C@H](N1)C(=O)OCC(C)(C)C2] |
| AB133 | 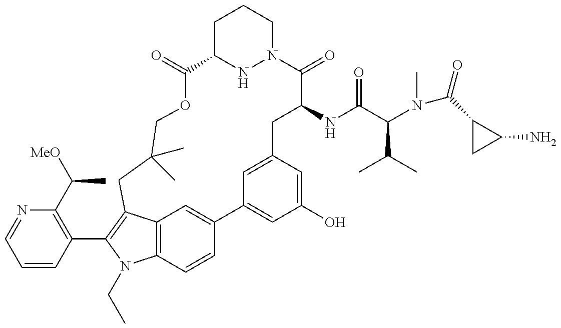 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@H]1N)C(=O)N1CCC[C@H](N1)C(=O)OCC(C)(C)C2] |
| AB134 | 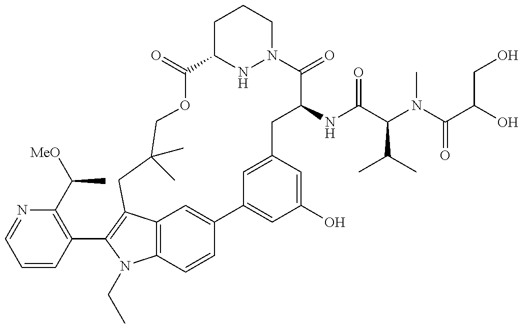 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(O)CO)C(=O)N1CCC[C@H](N1)C(=O)OCC(C)(C)C2] |
| AB135 | 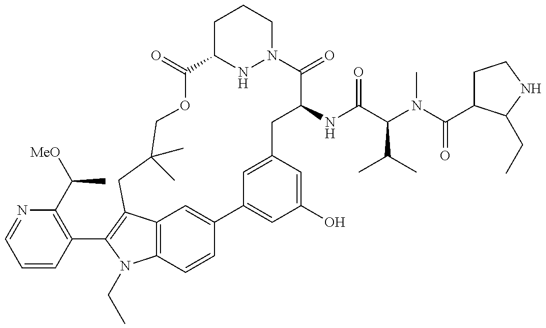 [SMILES: CCC1NCCC1C(=O)N(C)[C@H](C(=O)N[C@H]1Cc2cc(O)cc(c2)-c2ccc3c(c2)c(c(-c2cccnc2[C@H](C)OC)n3CC)CC(C)(C)COC(=O)[C@@H]2CCCN(N2)C1=O)C(C)C] |
| AB137 | 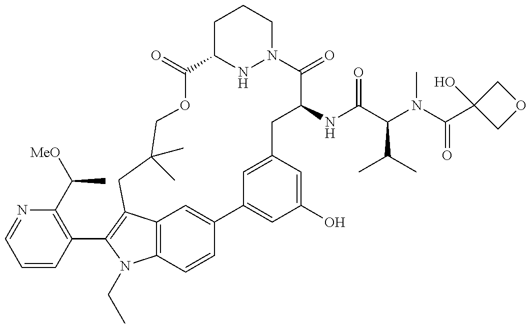 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AB138 | 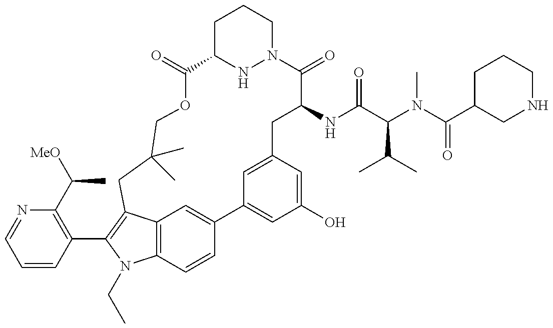 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCCNC1)C(=O)N1CCC[C@H](N1)C(=O)OCC(C)(C)C2] |
| AB139 | 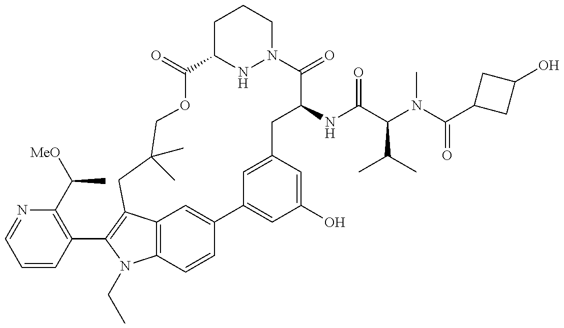 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC(O)C1)C(=O)N1CCC[C@H](N1)C(=O)OCC(C)(C)C2] |
| AB141 | 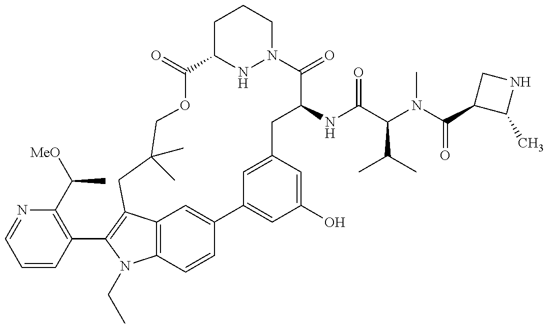 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CN[C@@H]1C)C(=O)N1CCC[C@H](N1)C(=O)OCC(C)(C)C2] |
| AB143 | 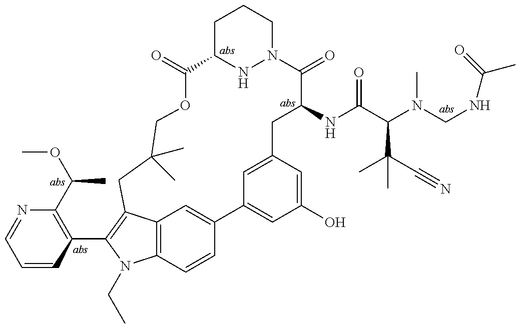 |
| AB144 | 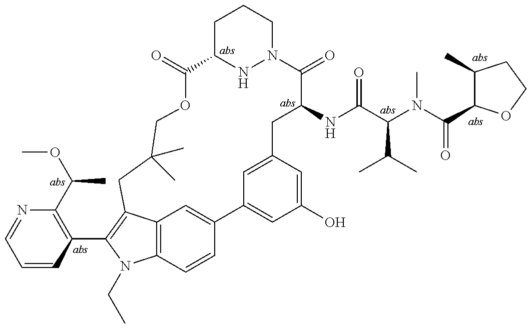 |
| AB145 | 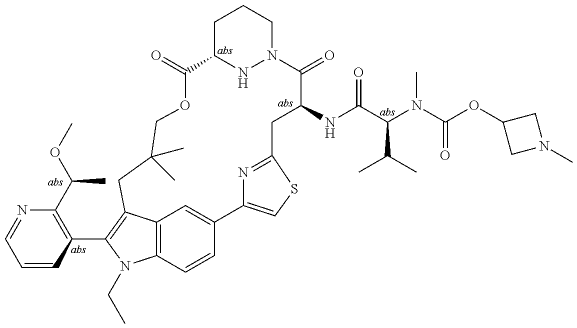 |
| AB146 | 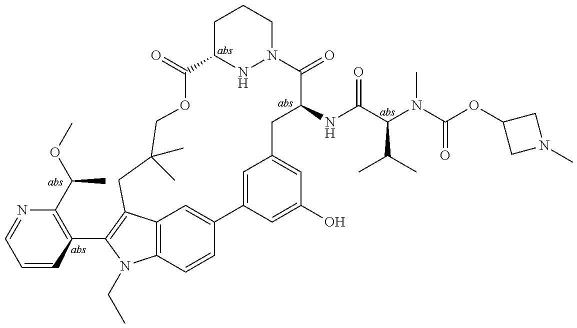 |
| AB147 | 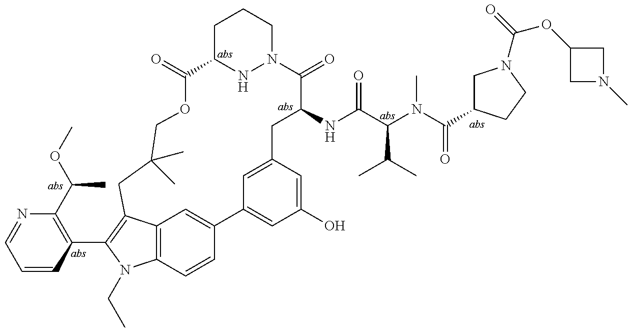 |
| AB148 | 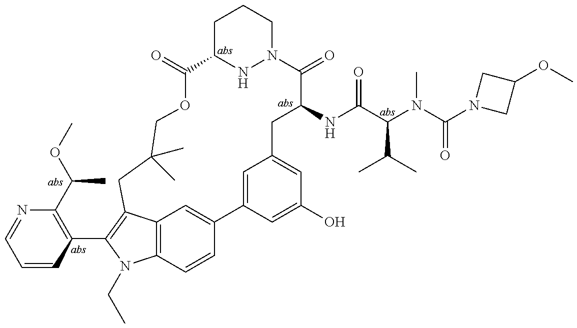 |
| AB149 | 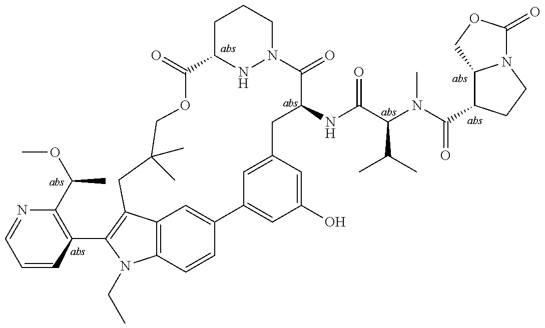 |
| AB150 | 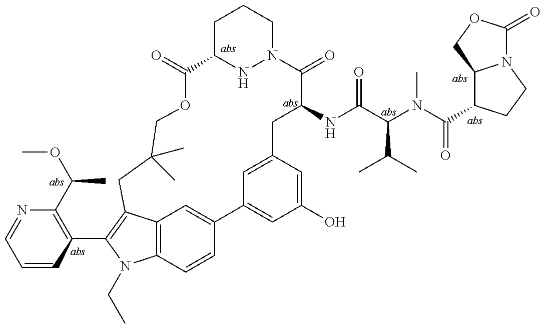 |
| AB151 | 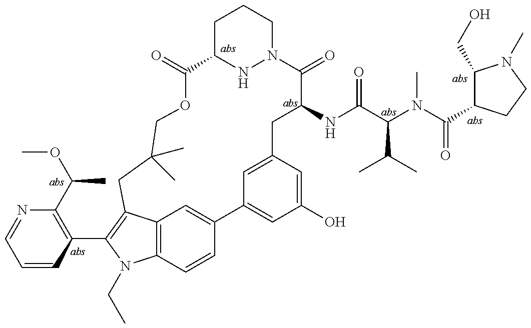 |
| AB152 | 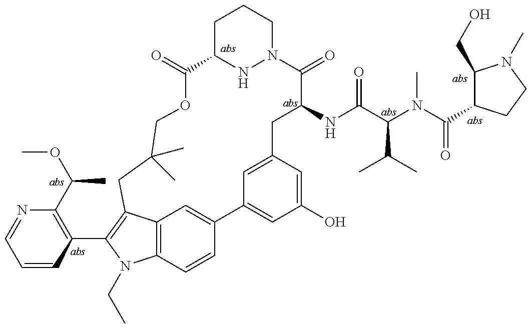 |
| AB153 | 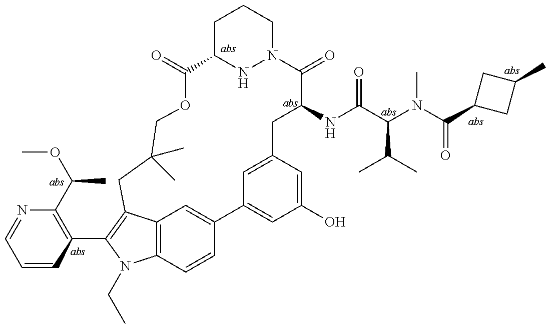 |
| AB154 | 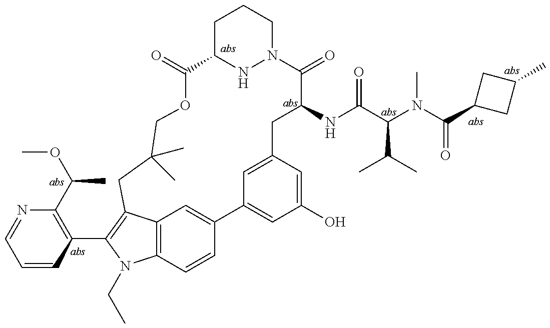 |
| AB155 | 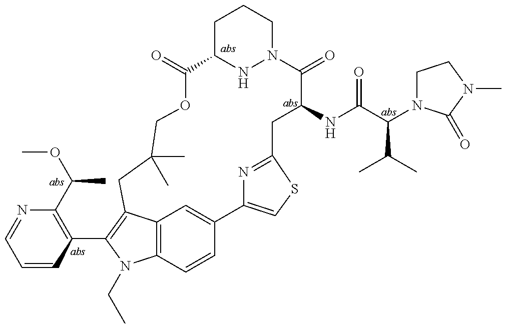 |
| AB156 | 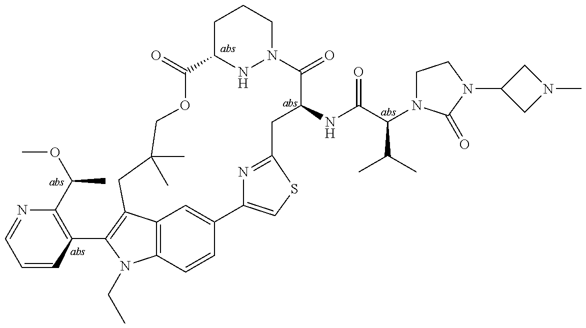 |
| AB157 | 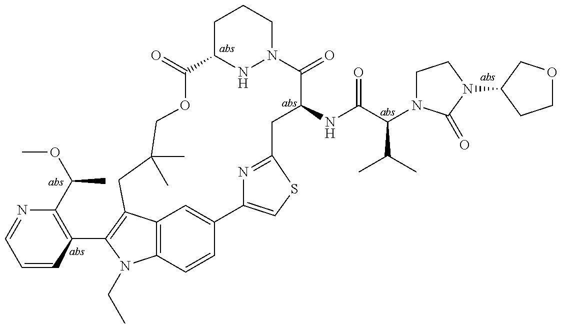 |
| AB158 | 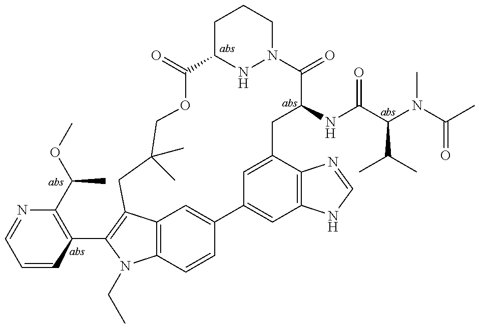 |
| AB159 | 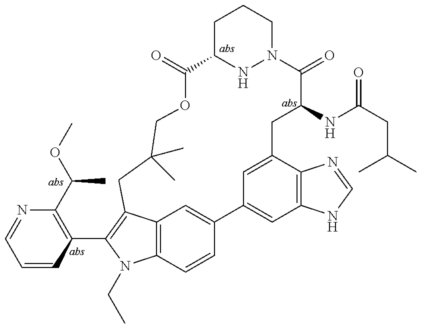 |
| AB160 | 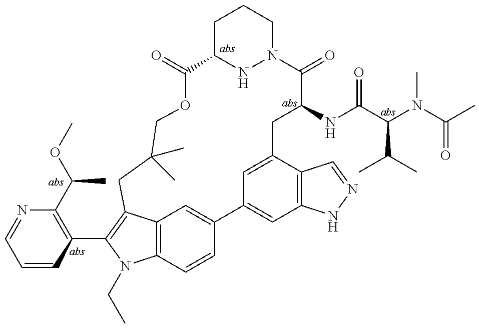 |
| AB161 | 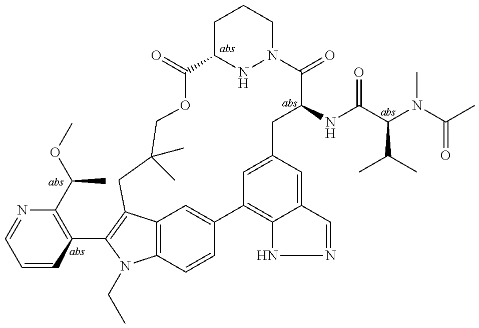 |
| AB162 | 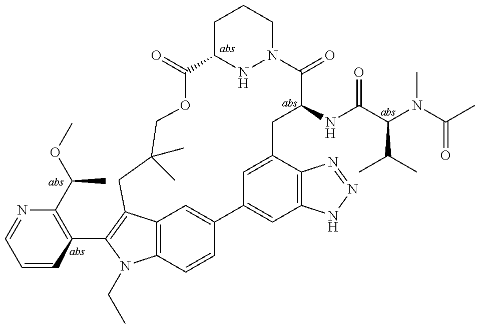 |
| AB163 | 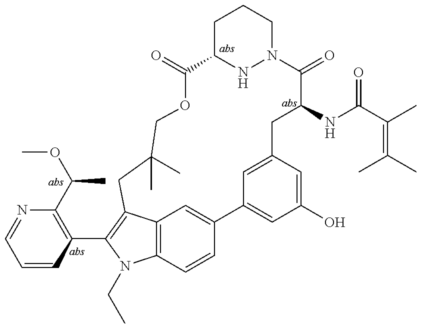 |
| AB164 | 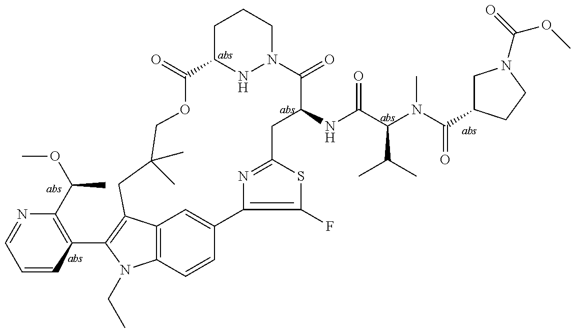 |
| AB165 | 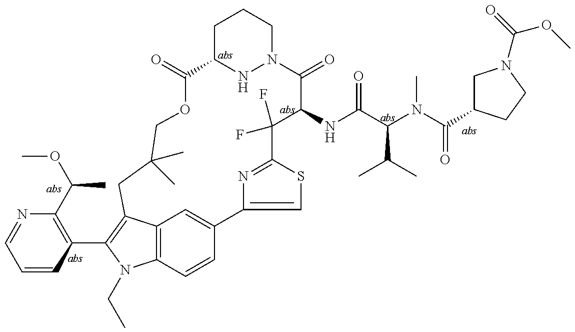 |
| AB166 | 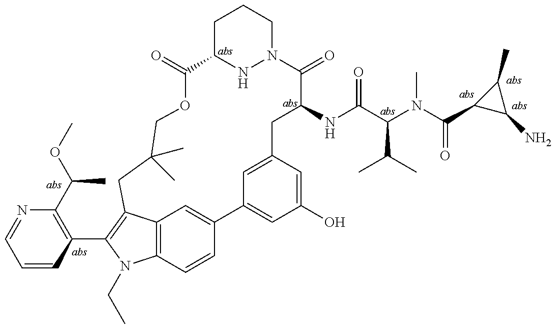 |
| AB167 | 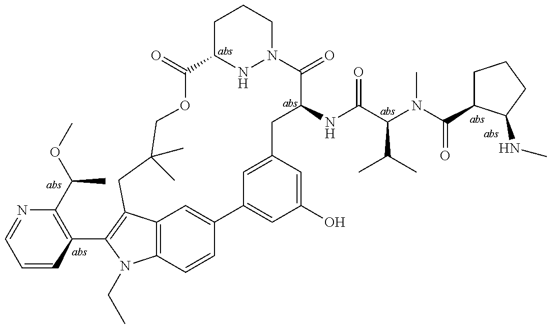 |
| AB168 | 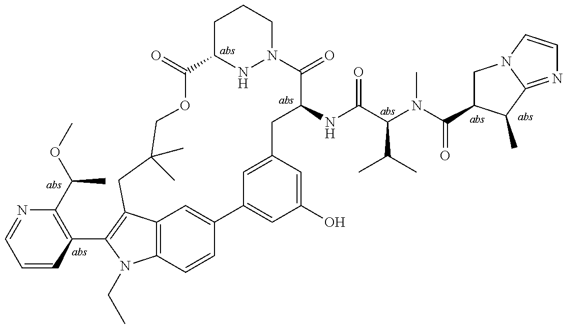 |
| AB169 | 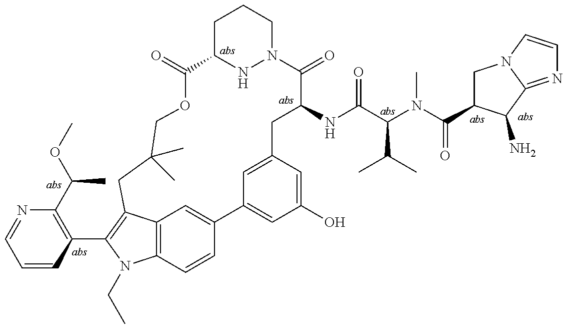 |
| AB170 | 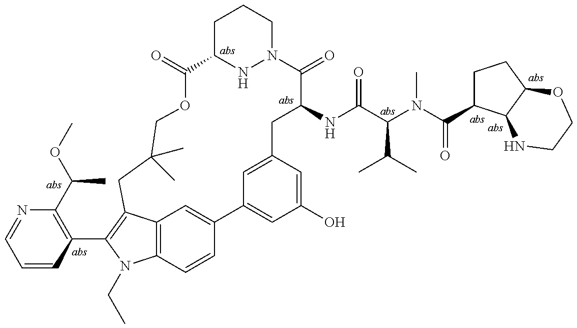 |
| AB171 | 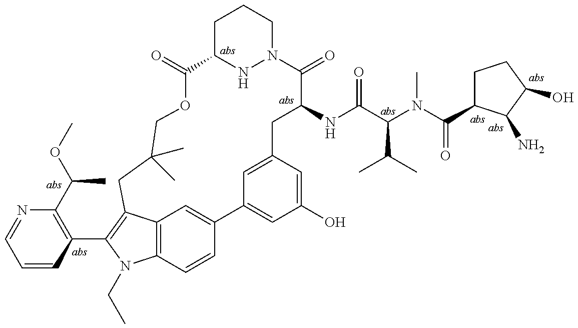 |
| AB172 | 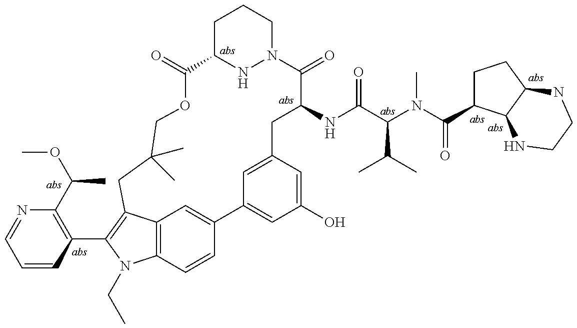 |
| AB173 | 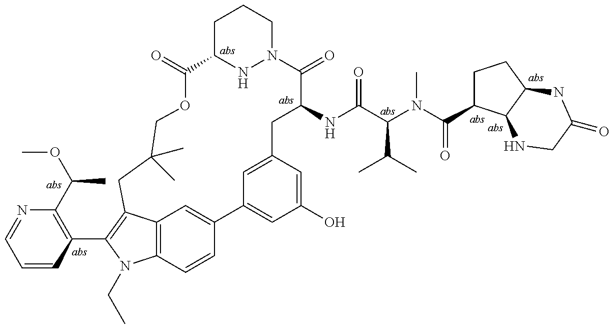 |
| AB174 | 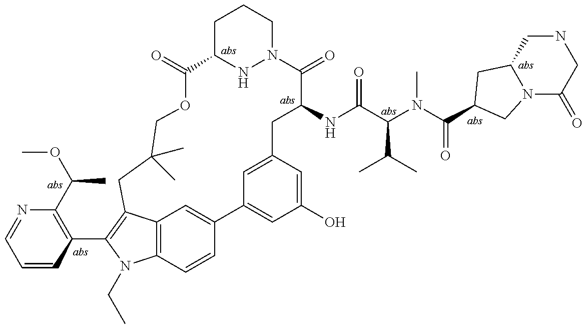 |
| AB175 | 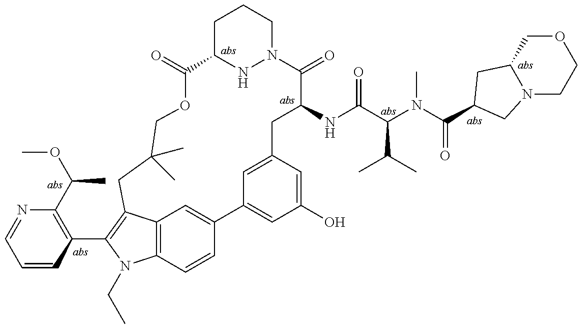 |
| AB176 | 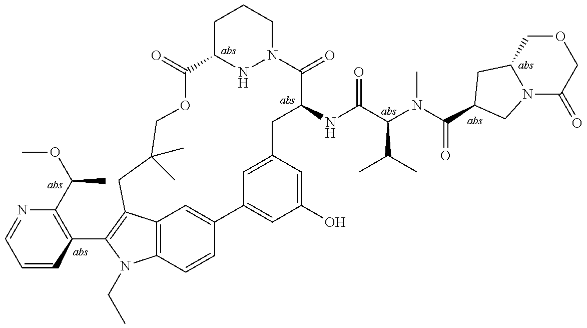 |
| AB177 | 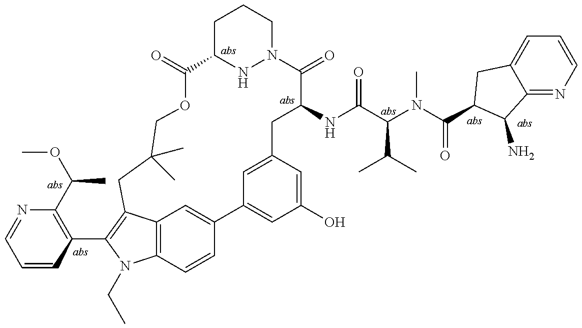 |
| AB178 | 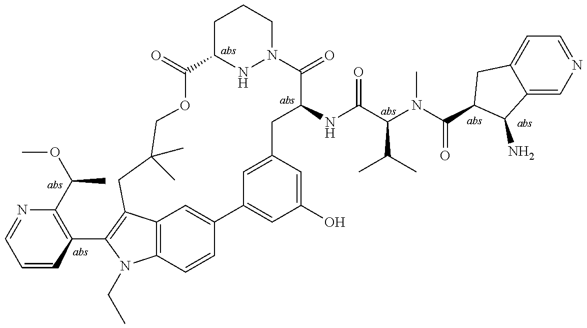 |
| AB179 | 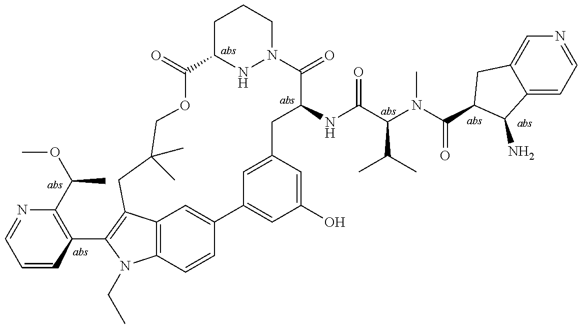 |
| AB180 | 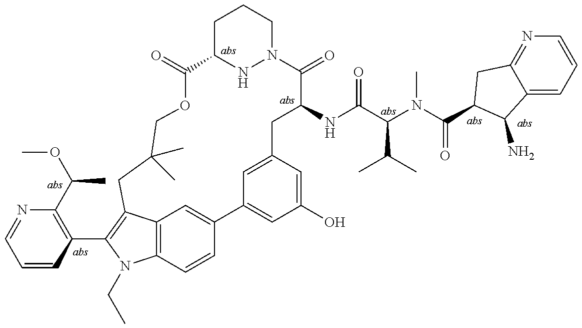 |
| AB181 | 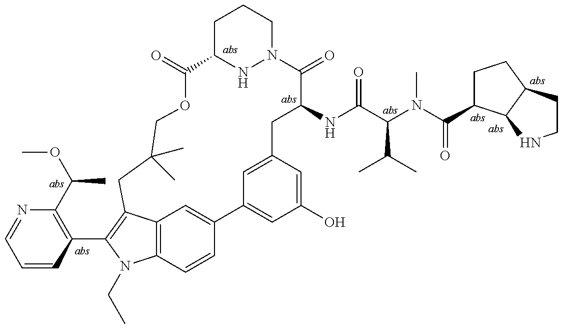 |
| AB182 | 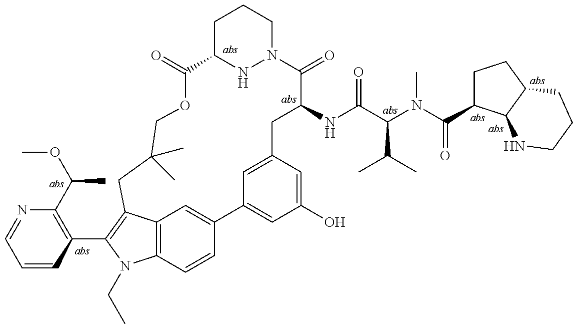 |
| AB183 | 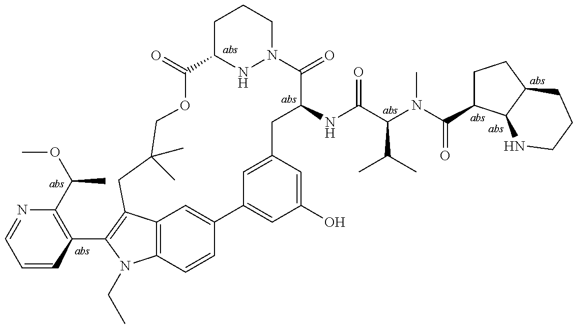 |
| AB184 | 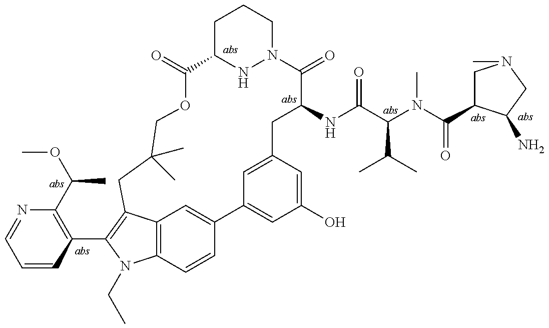 |
| AB185 | 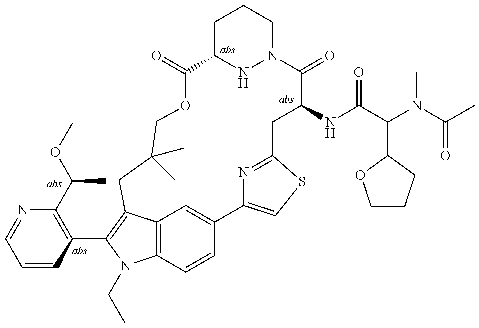 |
| AB186 | 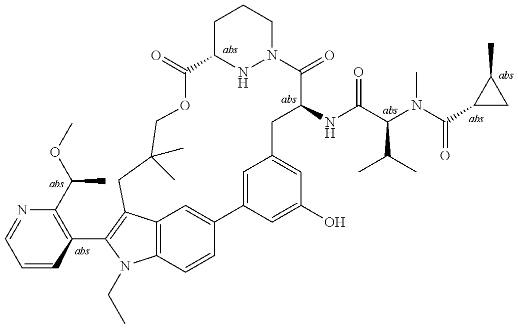 |
| AB187 | 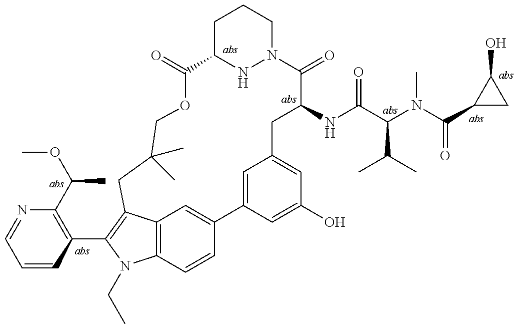 |
| AB188 | 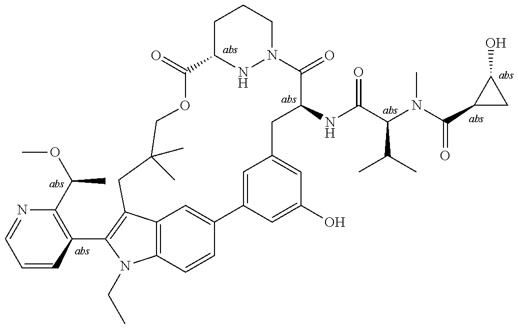 |
| AB189 | 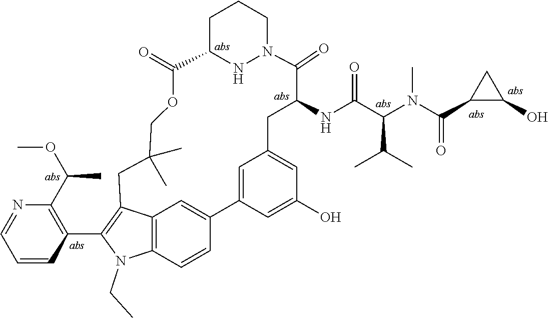 |
| AB190 | 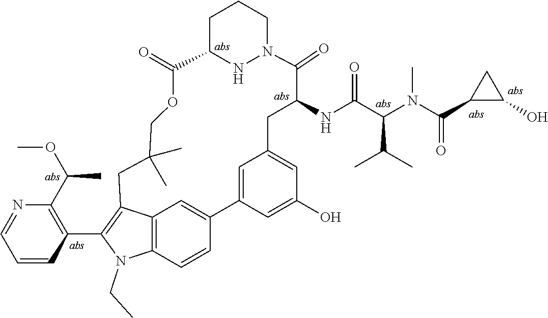 |
| AB191 | 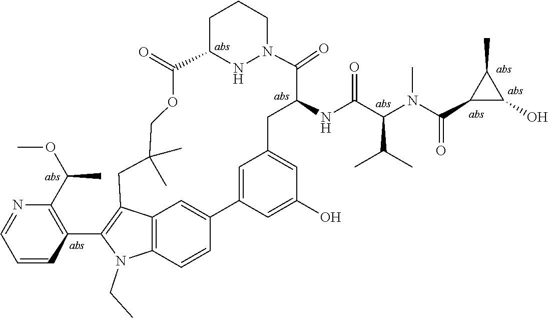 |
| AB192 | 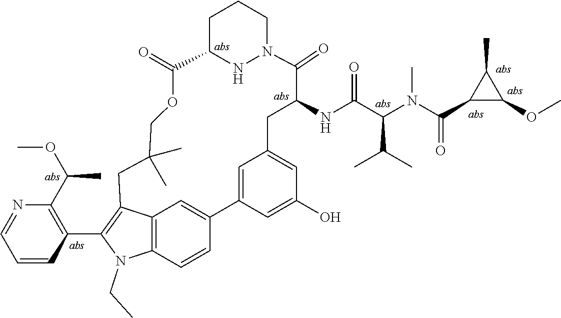 |
| AB193 | 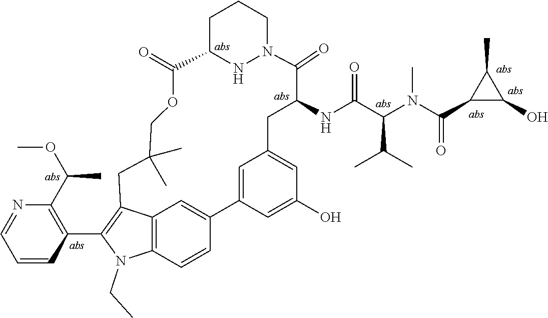 |
| AB194 | 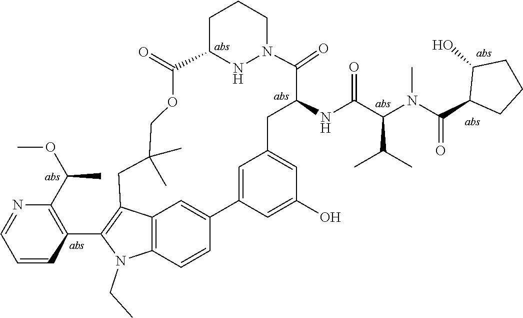 |
| AB195 | 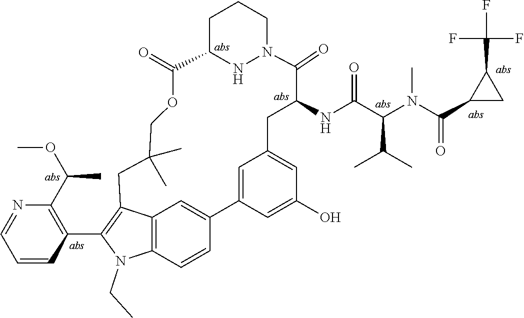 |
| AB196 | 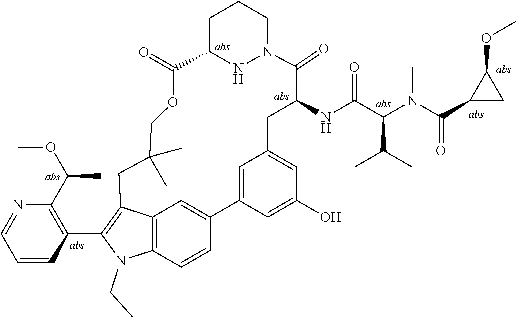 |
| AB197 | 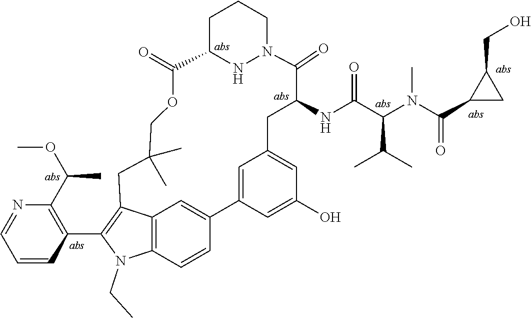 |
| AB198 | 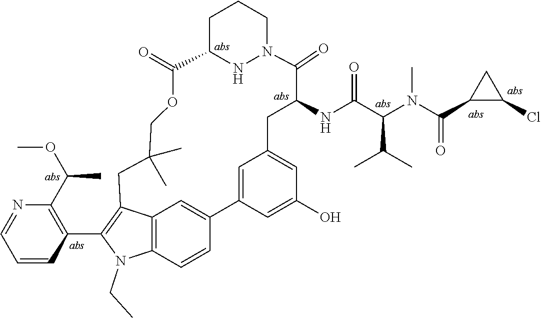 |
| AB199 | 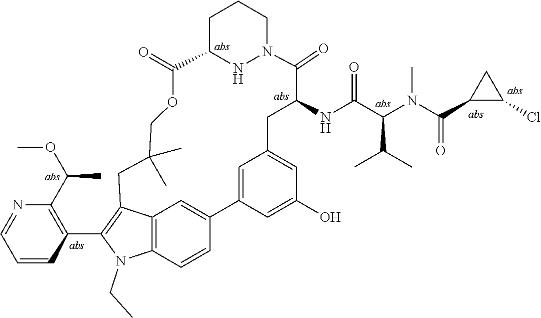 |
| AB200 | 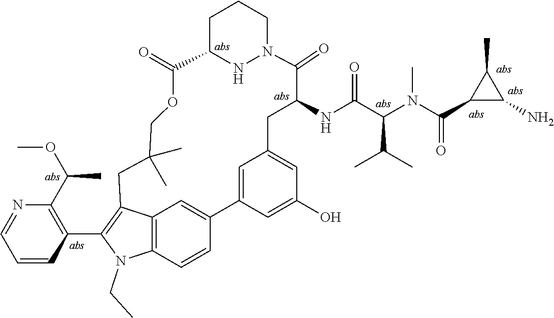 |
| AB201 | 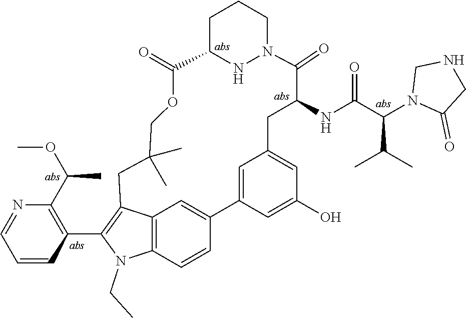 |
| AB202 | 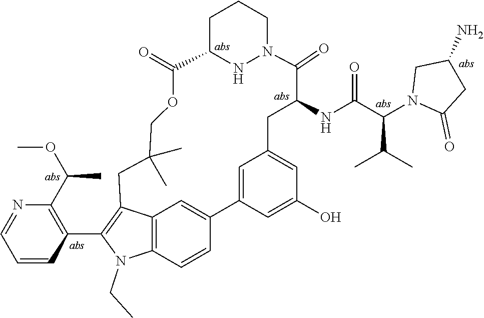 |
| AB203 | 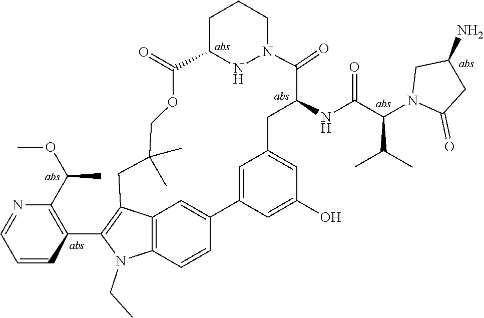 |
| AB204 | 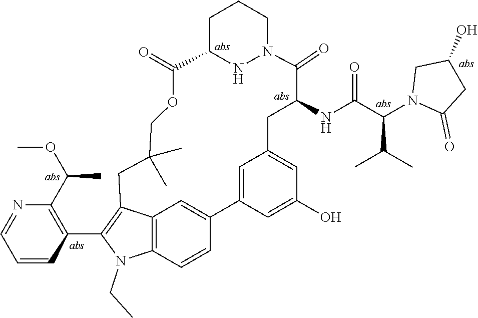 |
| AB205 | 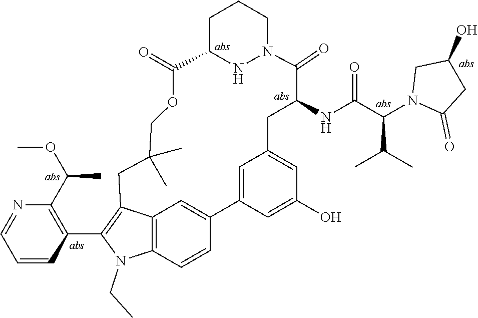 |
| AB206 | 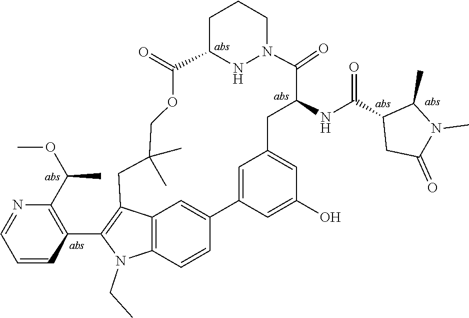 |
| AB207 | 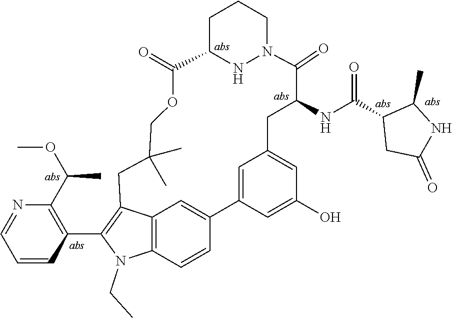 |
| AB208 | 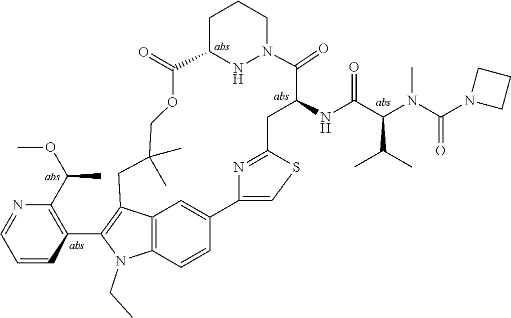 |
| AB209 | 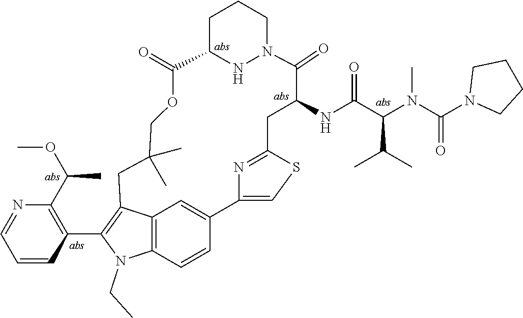 |
| AB210 | 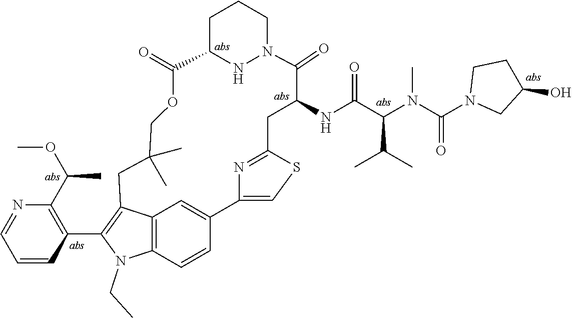 |
| AB211 | 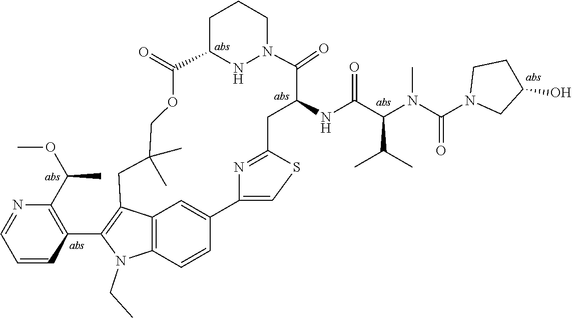 |
| AB212 | 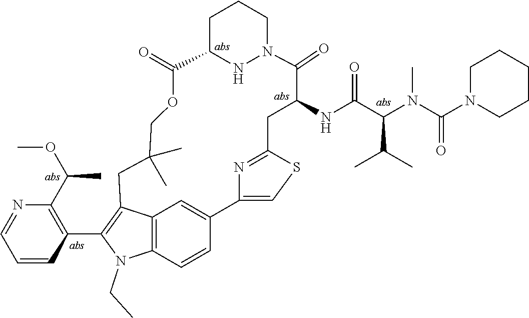 |
| AB213 | 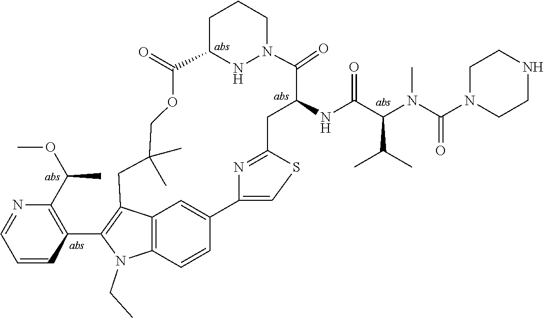 |
| AB214 | 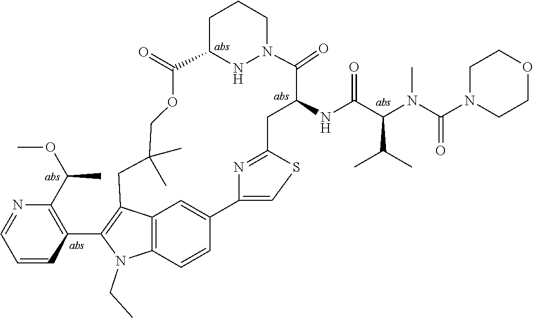 |
| AB215 | 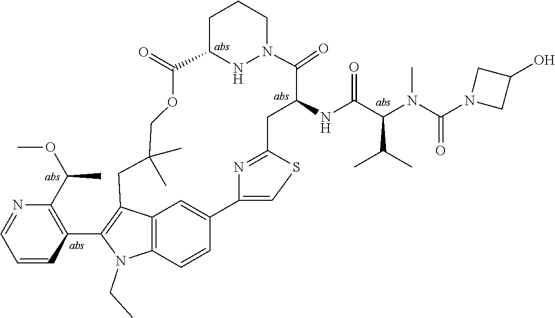 |
| AB216 | 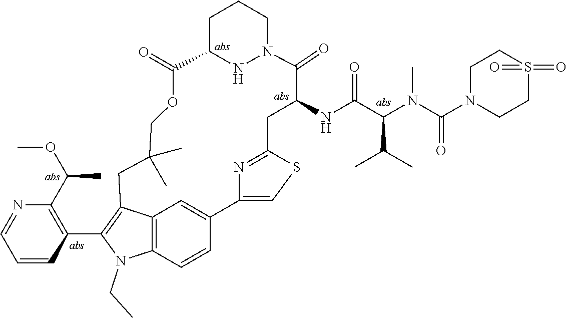 |
| AB217 | 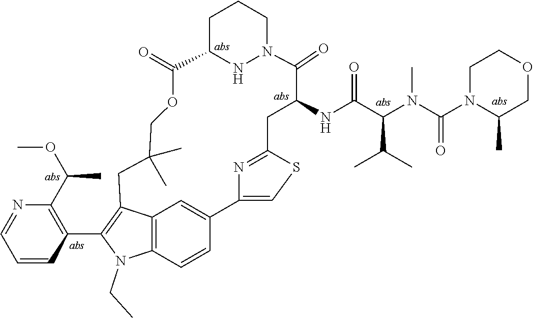 |
| AB218 | 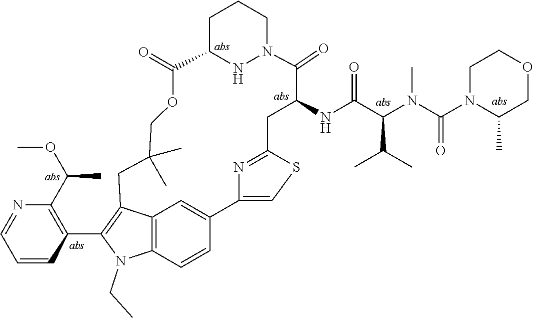 |
| AB219 | 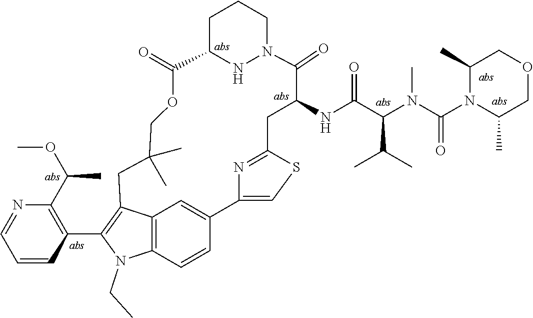 |
| AB220 | 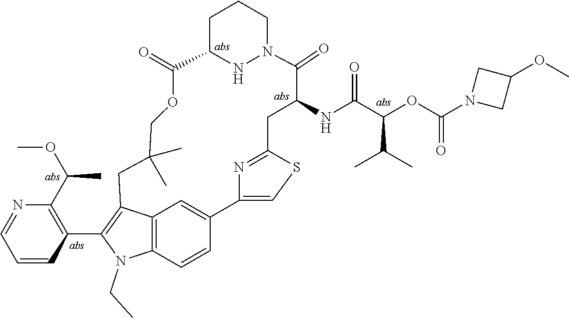 |
| AB221 | 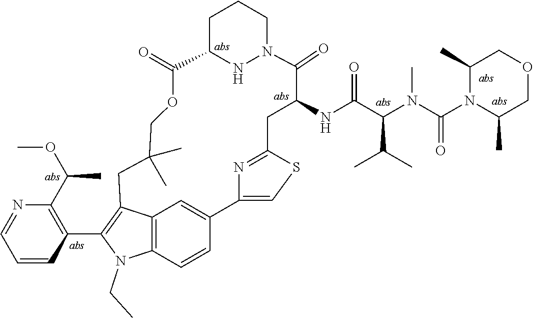 |
| AB222 | 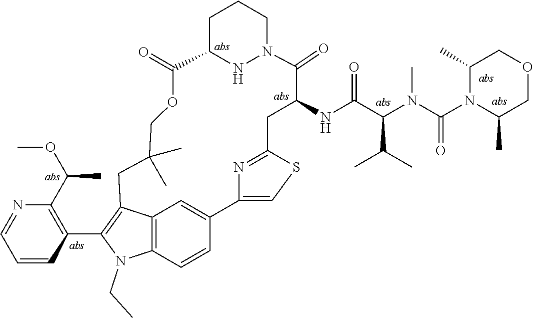 |
| AB223 | 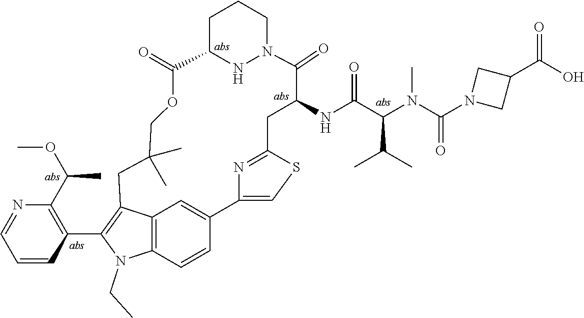 |
| AB224 | 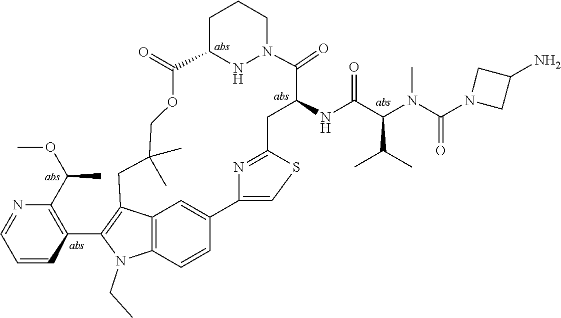 |
| AB225 | 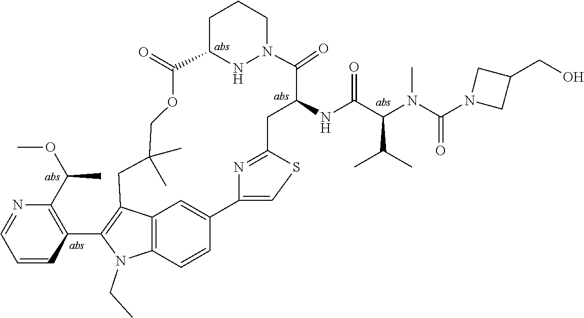 |
| AB226 | 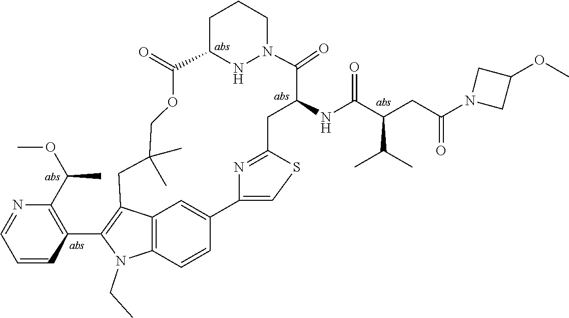 |
| AB227 | 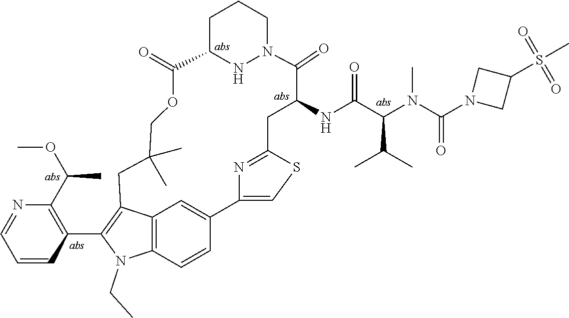 |
| AB228 | 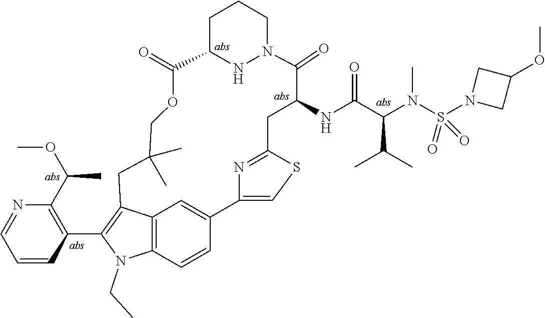 |
| AB229 | 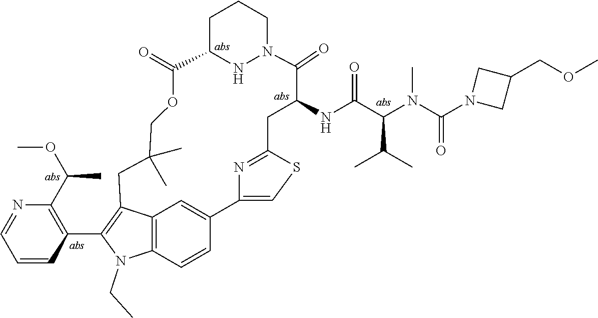 |
| AB230 | 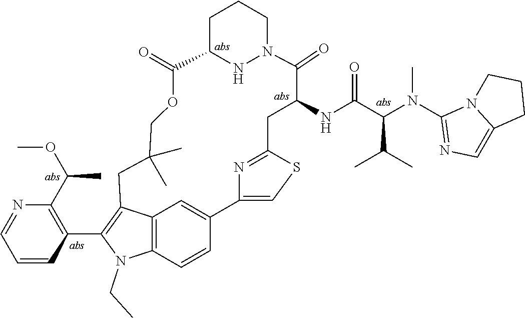 |
| AB231 | 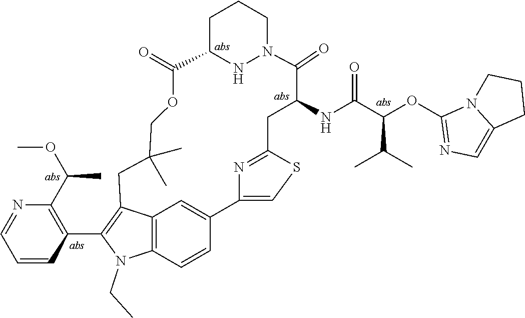 |
| AB232 | 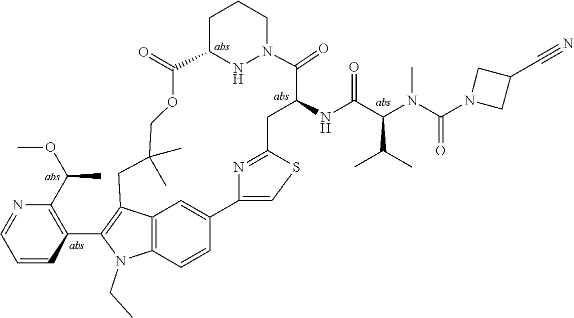 |
| AB233 | 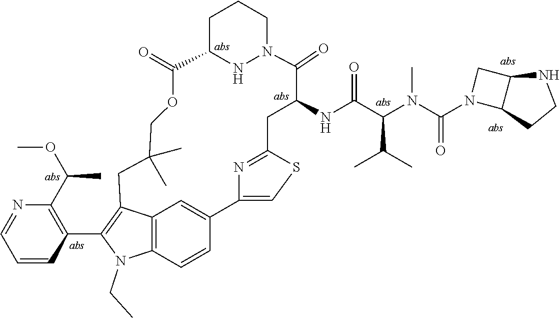 |
| AB234 | 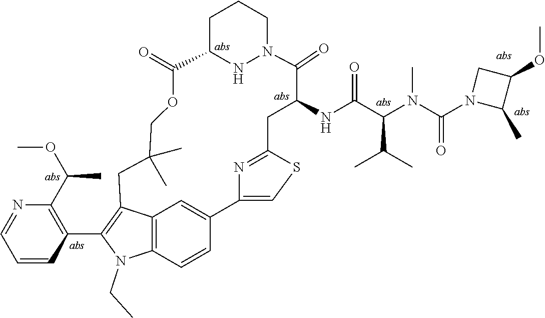 |
| AB235 | 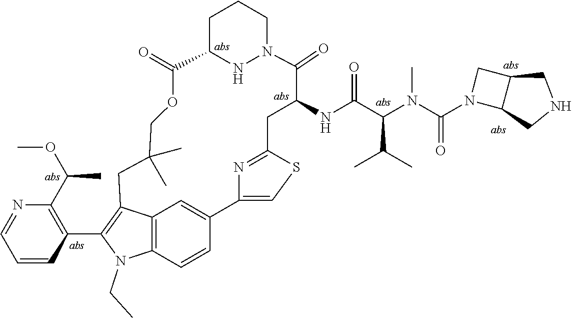 |
| AB236 | 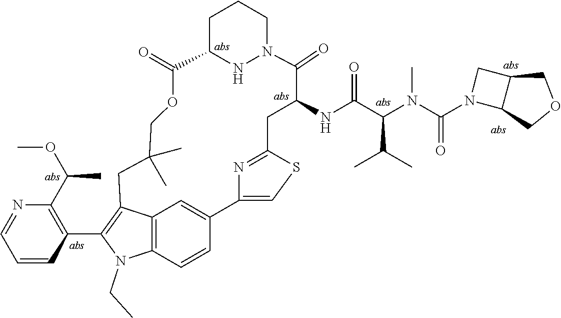 |
| AB237 | 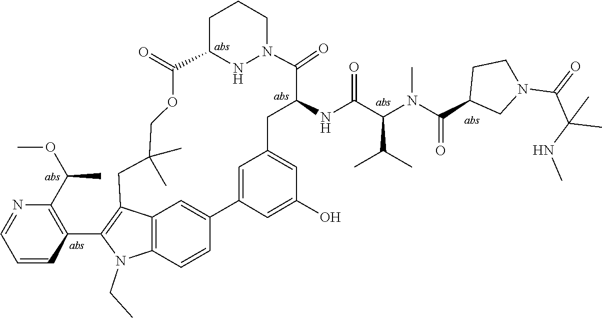 |
| AB238 | 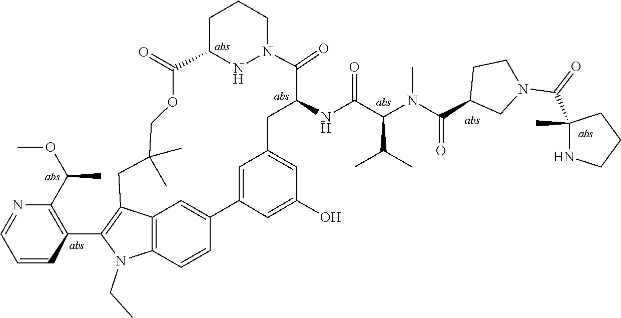 |
| AB239 | 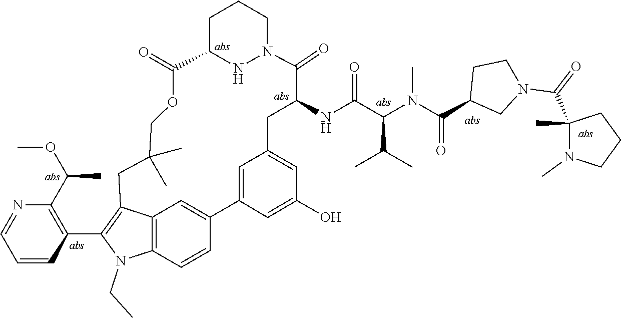 |
| AB240 | 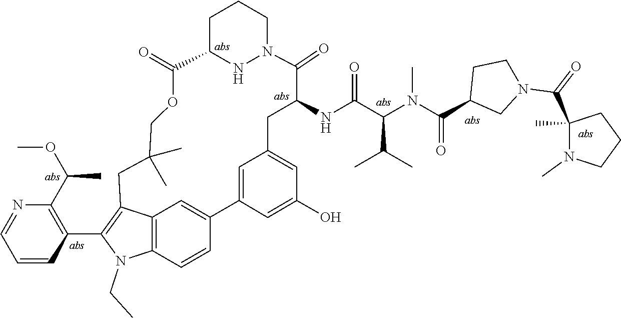 |
| AB241 | 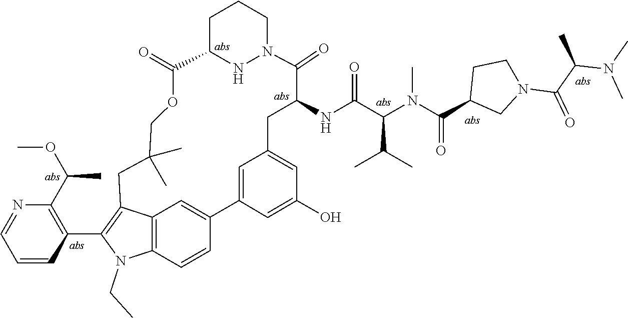 |
| AB242 | 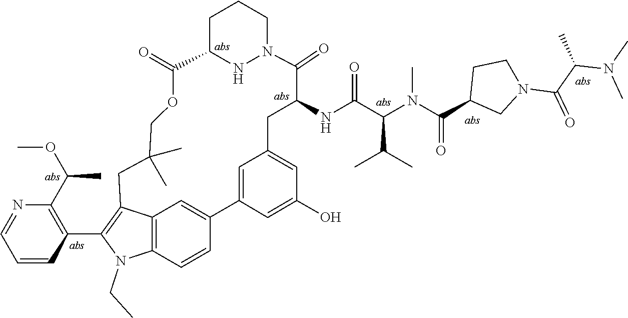 |
| AB243 | 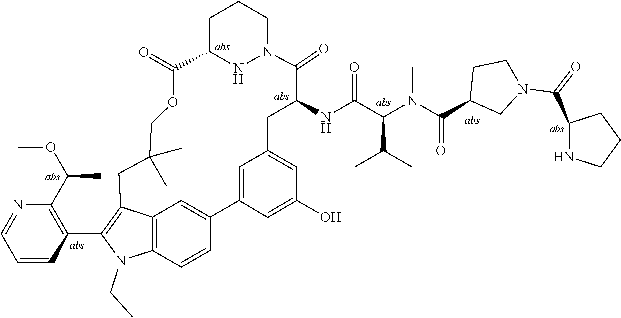 |
| AB244 | 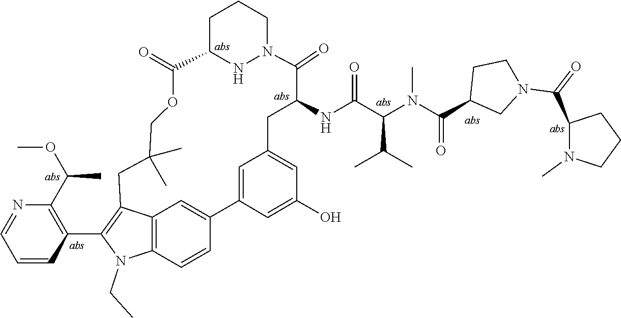 |
| AB245 | 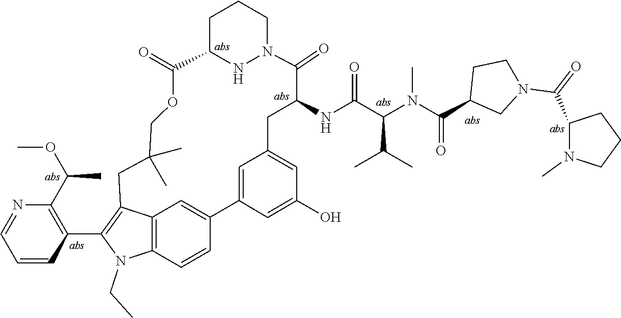 |
| AB246 | 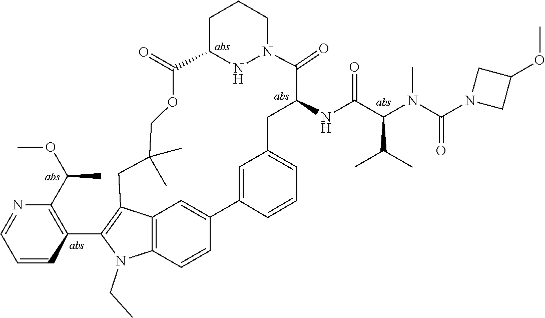 |
| AB247 | 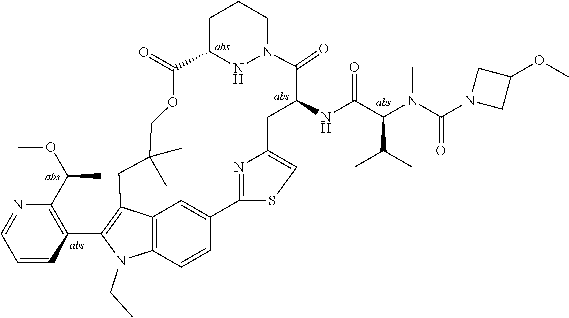 |
| AB248 | 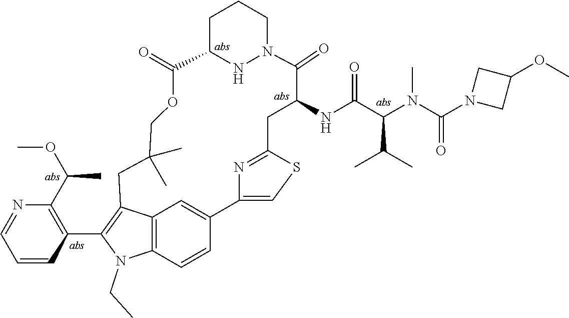 |
| AB249 | 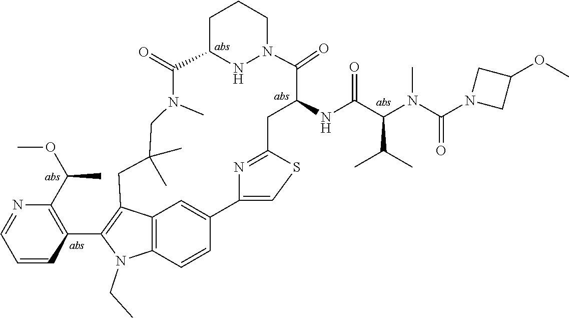 |
| AB250 | 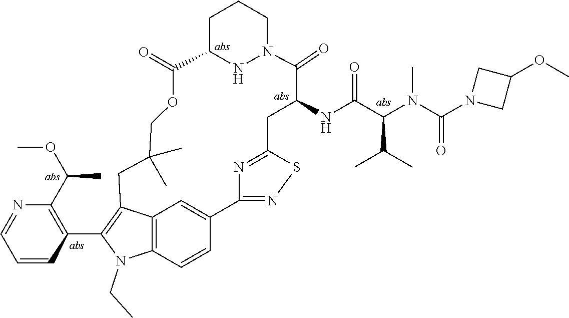 |
| AB251 | 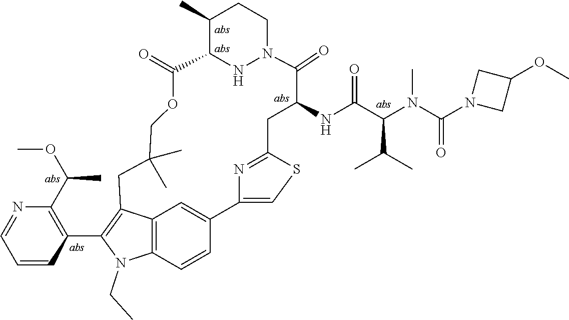 |
| AB252 | 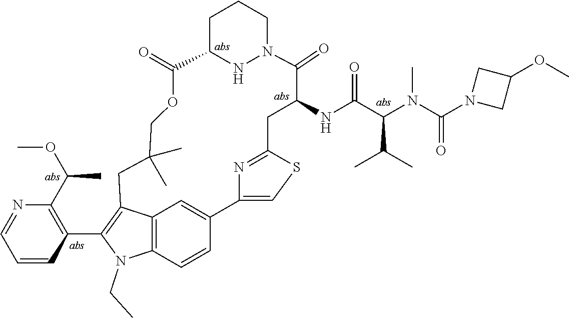 |
| AB253 | 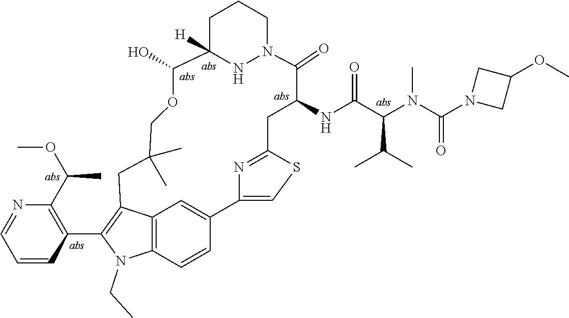 |
| AB254 | 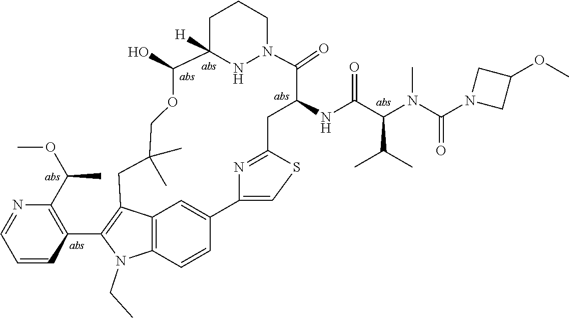 |
| AB255 | 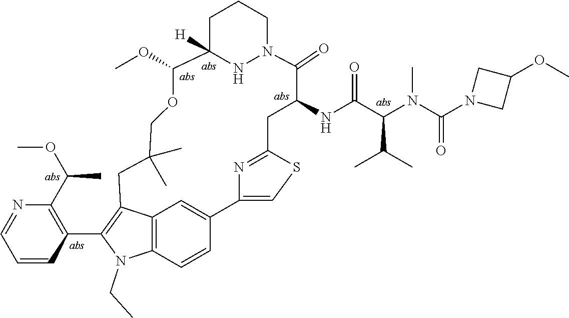 |
| AB256 | 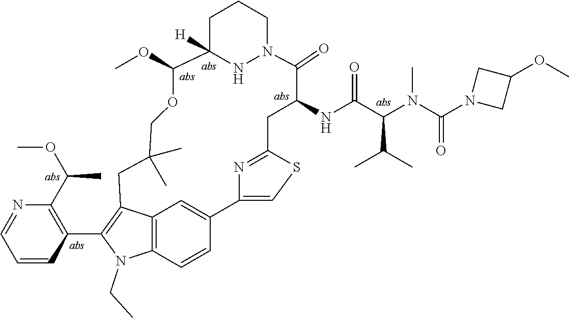 |
| AB257 | 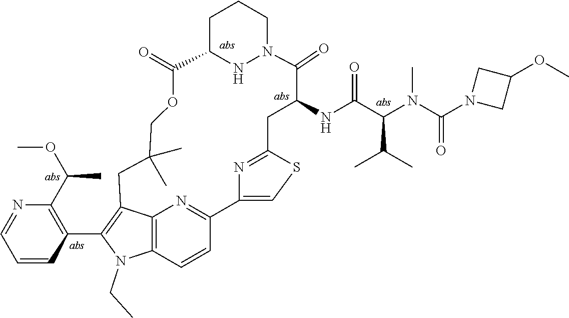 |
| AB258 | 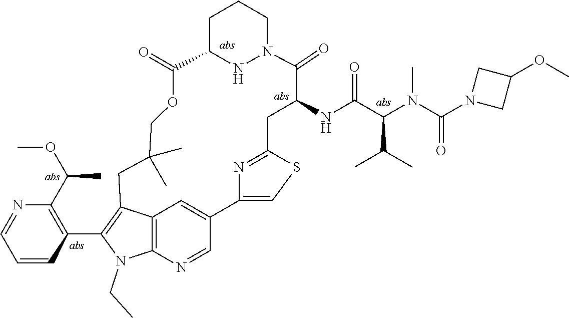 |
| AB259 | 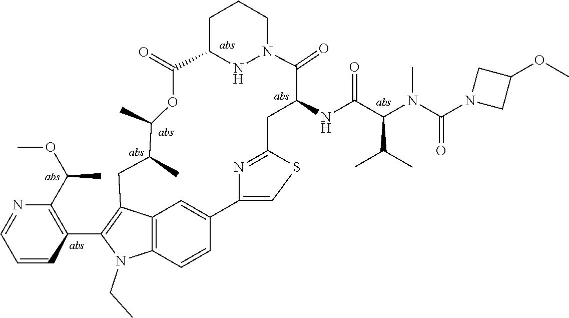 |
| AB260 | 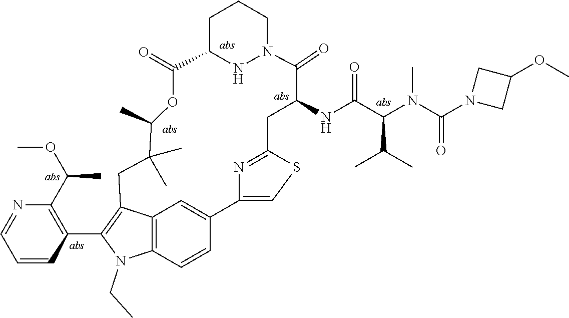 |
| AB261 | 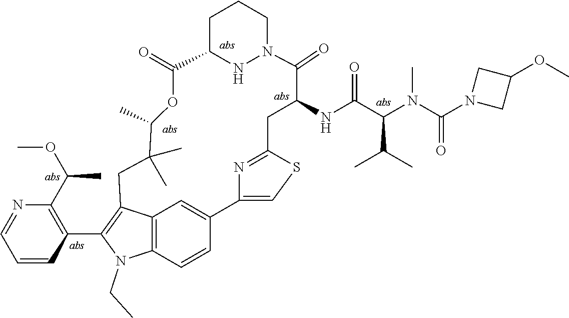 |
| AB262 | 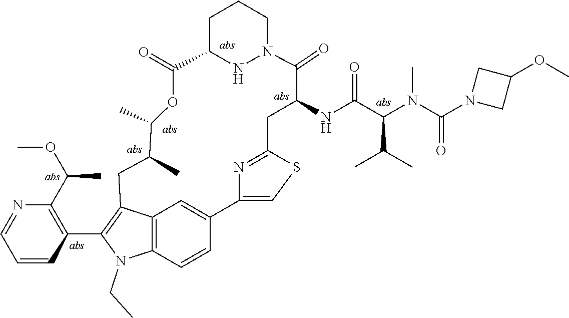 |
| AB263 | 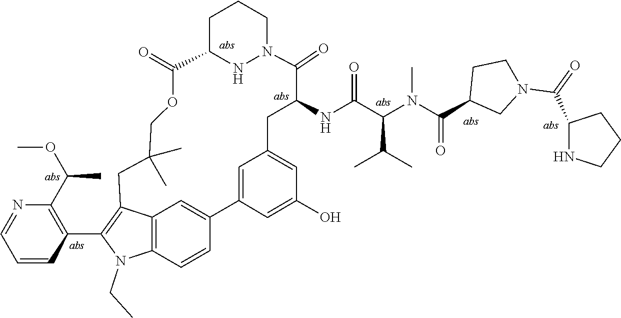 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below and in WO 2021/091956, together with synthetic methods known in the art of synthetic organic chemistry, or variations thereon as appreciated by those skilled in the art. These methods include but are not limited to those methods described in the Schemes below or as described in WO 2021/091956.

Compounds of Table A1 herein were prepared using methods disclosed herein or were prepared using methods disclosed herein combined with the knowledge of one of skill in the art. Compounds of Table A2 may be prepared using methods disclosed herein or may be prepared using methods disclosed herein combined with the knowledge of one of skill in the art.

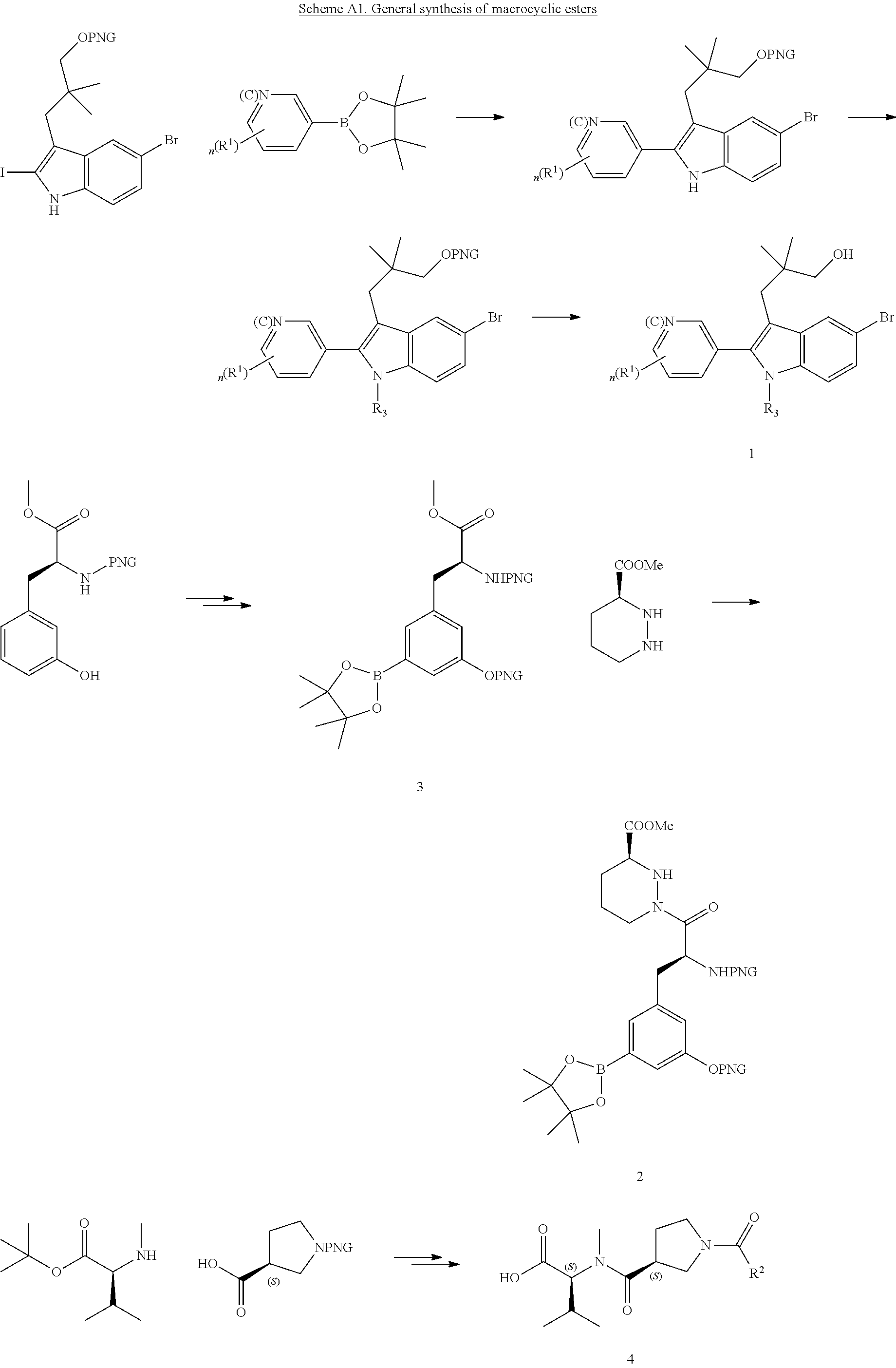

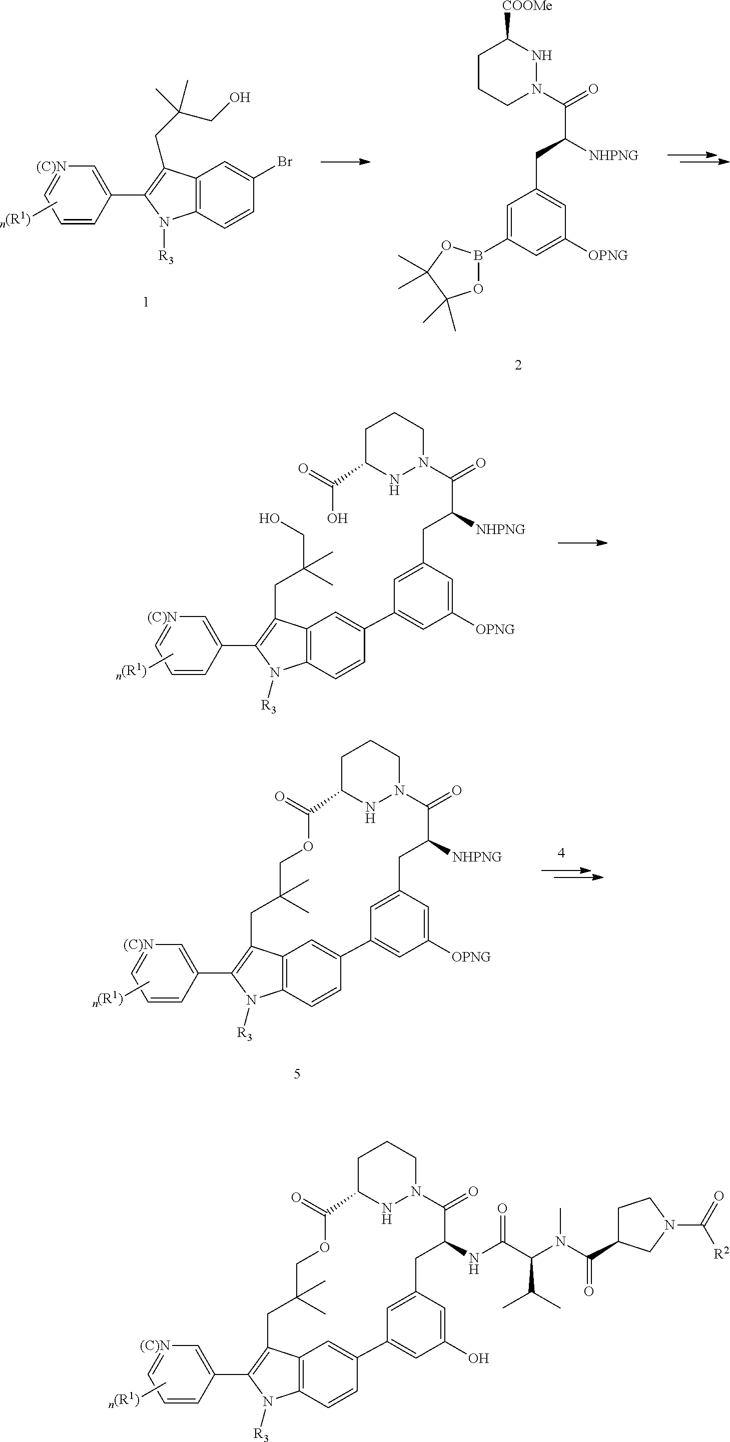

A general synthesis of macrocyclic esters is outlined in Scheme A1. An appropriately substituted Aryl Indole intermediate (1) can be prepared in three steps starting from protected 3-(5-bromo-2-iodo-1H-indol-3-yl)-2,2-dimethylpropan-1-ol and appropriately substituted boronic acid, including Palladium mediated coupling, alkylation, and de-protection reactions.

Methyl-amino-hexahydropyridazine-3-carboxylate-boronic ester (2) can be prepared in three steps, including protection, Iridium catalyst mediated borylation, and coupling with methyl (S)-hexahydropyridazine-3-carboxylate.

An appropriately substituted acetylpyrrolidine-3-carbonyl-N-methyl-L-valine (4) can be made by coupling of methyl-L-valinate and protected (S)-pyrrolidine-3-carboxylic acid, followed by deprotection, coupling with an appropriately substituted carboxylic acid, and a hydrolysis step.

The final macrocyclic esters can be made by coupling of methyl-amino-hexahydropyridazine-3-carboxylate-boronic ester (2) and intermediate (1) in the presence of Pd catalyst followed by hydrolysis and macrolactonization steps to result in an appropriately protected macrocyclic intermediate (5). Deprotection and coupling with an appropriately substituted acetylpyrrolidine-3-carbonyl-N-methyl-L-valine (4) results in a macrocyclic product. Additional deprotection or functionalization steps are be required to produce a final compound. For example, a person of skill in the art would be able to install into a macrocyclic ester a desired —B-L-W group of a compound of Formula (AI), where B, L and W are defined herein, including by using methods exemplified in the Example section herein.

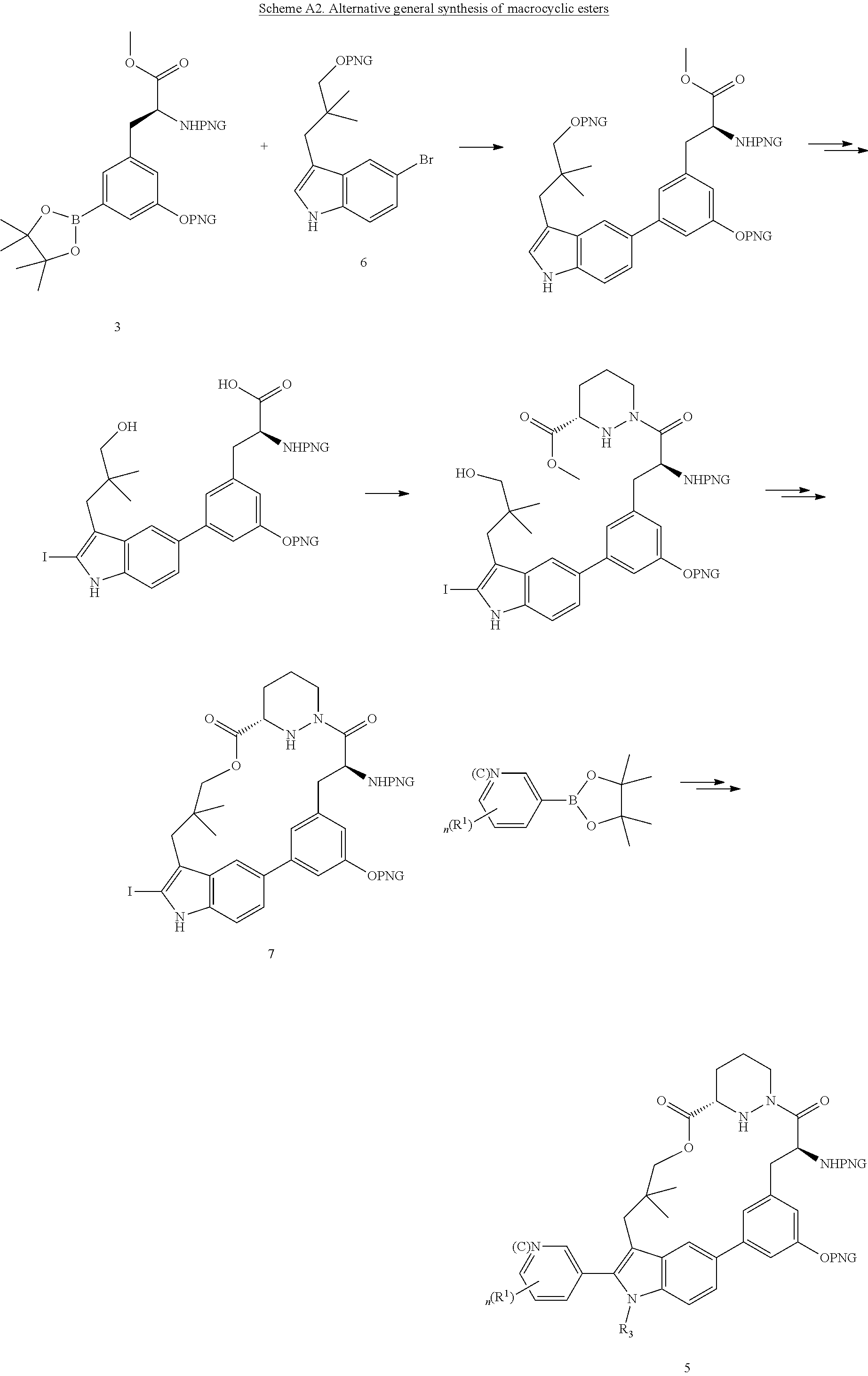

Alternatively, macrocyclic esters can be prepared as described in Scheme 2. An appropriately protected bromo-indolyl (6) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of Pd catalyst with an appropriately substituted boronic ester and alkylation can yield fully a protected macrocycle (5). Additional deprotection or functionalization steps are required to produce a final compound. For example, a person of skill in the art would be able to install into a macrocyclic ester a desired —B-L-W group of a compound of Formula (AI), where B, L and W are defined herein, including by using methods exemplified in the Example section herein.

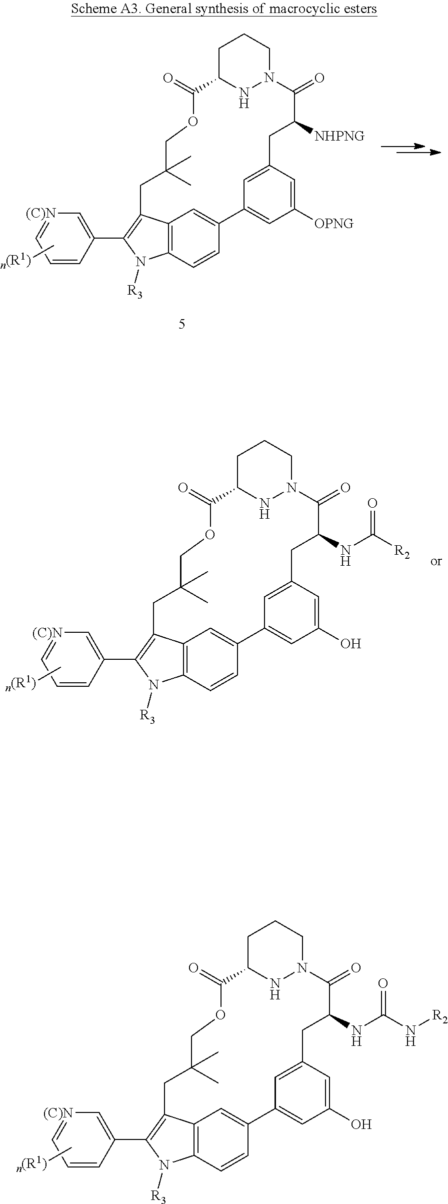

Alternatively, fully a protected macrocycle (5) can be deprotected and coupled with an appropriately substitututed coupling partners, and deprotected to results in a macrocyclic product. Additional deprotection or functionalization steps are be required to produce a final compound. For example, a person of skill in the art would be able to install into a macrocyclic ester a desired —B-L-W group of a compound of Formula (AI), where B, L and W are defined herein, including by using methods exemplified in the Example section herein.

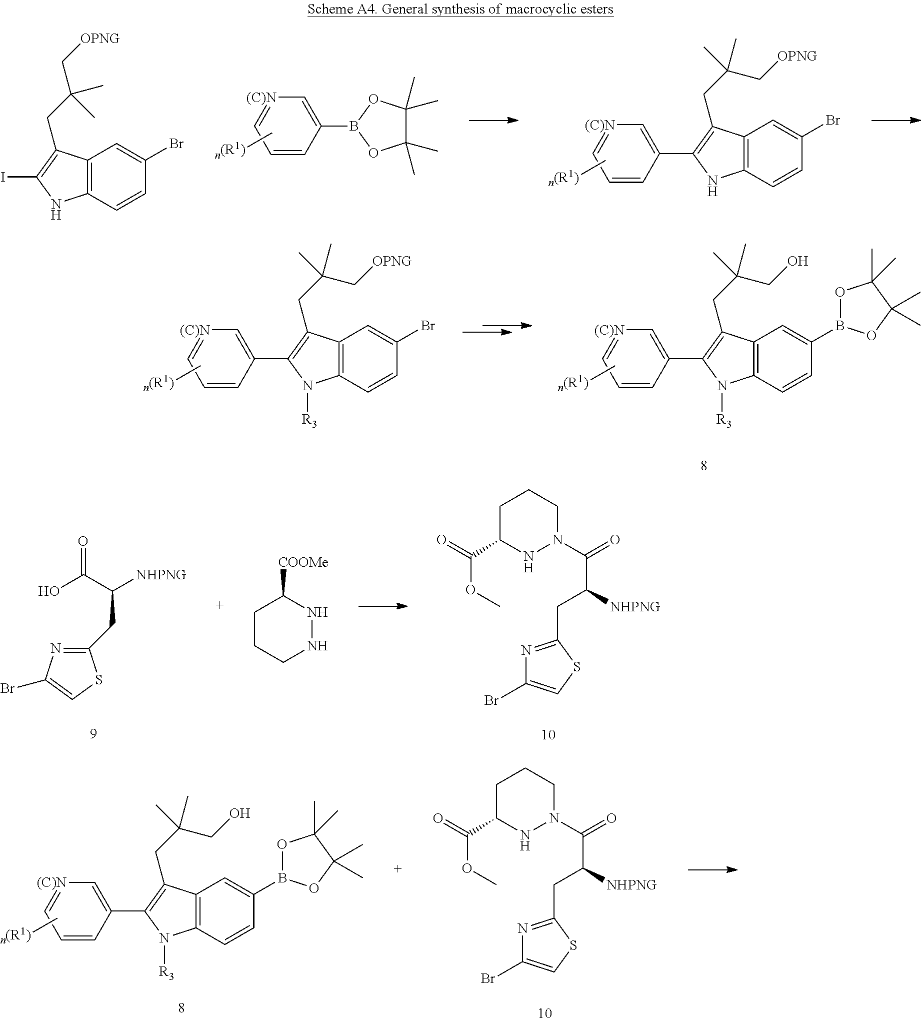

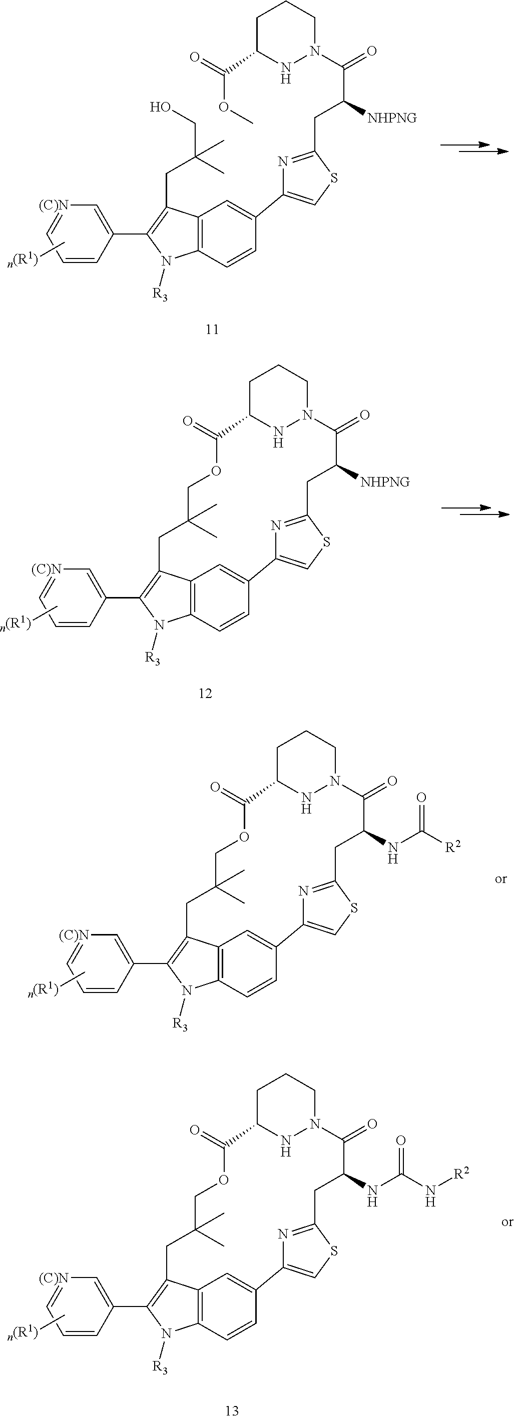

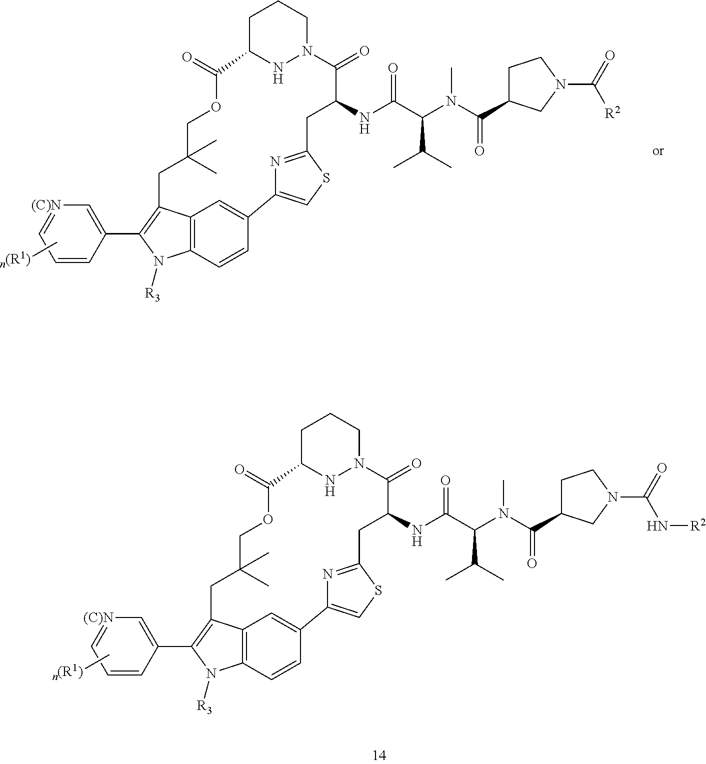

An alternative general synthesis of macrocyclic esters is outlined in Scheme A4. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted carboxylic acid (or other coupling partner) or intermediate 4 can result in a macrocyclic product. Additional deprotection or functionalization steps could be required to produce a final compound 13 or 14.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the WO 2021/091956. For example, a person of skill in the art would be able to install into a macrocyclic ester a desired —B-L-W group of a compound of Formula (AI), where B, L and W are defined herein, including by using methods exemplified in the Example section herein.

In some embodiments, the RAS inhibitor is a compound disclosed in WO 2021/091982.

In some embodiments, the RAS(ON) inhibitor is a compound, or a pharmaceutically acceptable salt thereof, having the structure of Formula BI:

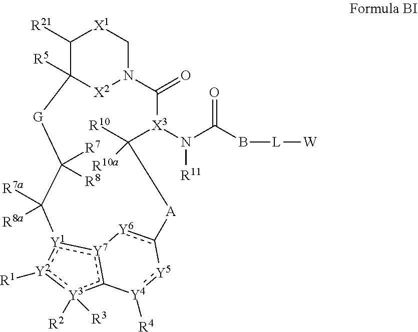

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI, R²¹ is hydrogen.

In some embodiments, provided herein is a compound, or pharmaceutically acceptable salt thereof, having the structure of Formula BIa:

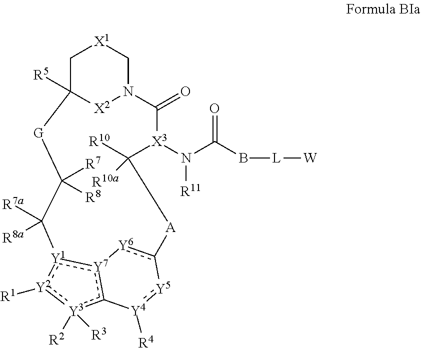

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula BIb:

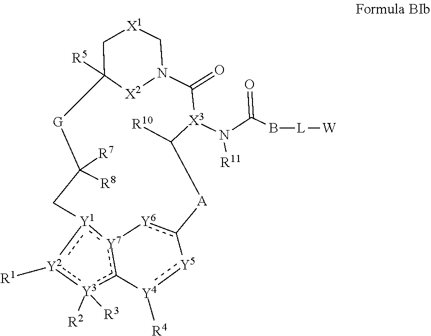

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, G is optionally substituted C₁-C₄ heteroalkylene.

In some embodiments, a compound having the structure of Formula BIc is provided, or a pharmaceutically acceptable salt thereof:

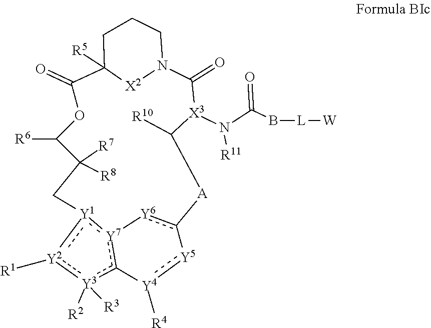

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, X² is NH. In some embodiments of Formula BI and subformula thereof, X³ is CH. In some embodiments of Formula BI and subformula thereof, R¹¹ is hydrogen. In some embodiments of Formula BI and subformula thereof, R¹¹ is C₁-C₃ alkyl. In some embodiments of Formula BI and subformula thereof, R¹¹ is methyl.

In some embodiments, the RAS(ON) inhibitor has the structure of Formula BId, or a pharmaceutically acceptable salt thereof:

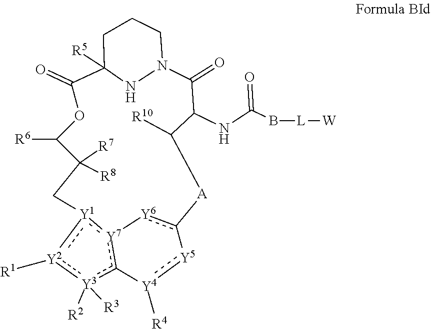

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments, the RAS inhibitor is a compound disclosed in WO 2021/091982.

In some embodiments of Formula BI and subformula thereof, X¹ is optionally substituted C₁-C₂ alkylene. In some embodiments, X¹ is methylene. In some embodiments of Formula BI and subformula thereof, X¹ is methylene substituted with a C₁-C₆ alkyl group or a halogen. In some embodiments, X¹ is —CH(Br)—. In some embodiments, X¹ is —CH(CH₃)—. In some embodiments of Formula BI and subformula thereof, R⁵ is hydrogen. In some embodiments of Formula BI and subformula thereof, R⁵ is C₁-C₄ alkyl optionally substituted with halogen. In some embodiments, R⁵ is methyl. In some embodiments of Formula BI and subformula thereof, Y⁴ is C. In some embodiments of Formula BI and subformula thereof, R⁴ is hydrogen. In some embodiments of Formula BI and subformula thereof, Y⁵ is CH.

In some embodiments of Formula BI and subformula thereof, Y⁶ is CH. In some embodiments of Formula BI and subformula thereof, Y¹ is C. In some embodiments of Formula BI and subformula thereof, Y² is C. In some embodiments of Formula BI and subformula thereof, Y³ is N. In some embodiments of Formula BI and subformula thereof, R³ is absent. In some embodiments of Formula BI and subformula thereof, Y⁷ is C.

In some embodiments, the RAS(ON) inhibitor has the structure of Formula BIe, or a pharmaceutically acceptable salt thereof:

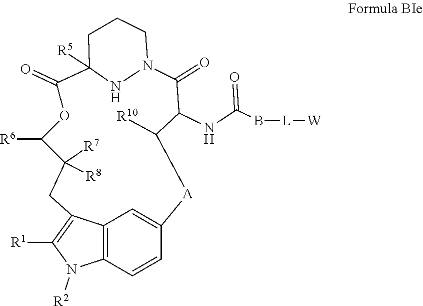

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula BI and subformula thereof, R⁶ is hydrogen. In some embodiments, R² is hydrogen, cyano, optionally substituted C1-C₆ alkyl, optionally substituted 3 to 6-membered cycloalkyl, or optionally substituted 3 to 6-membered heterocycloalkyl. In some embodiments, R² is optionally substituted C₁-C₆ alkyl. In some embodiments, R₂ is fluoroalkyl. In some embodiments, R² is ethyl. In some embodiments, R₂ is —CH₂CF₃. In some embodiments, R₂ is C₂-C₆ alkynyl. In some embodiments, R₂ is —CHC≡CH. In some embodiments, R₂ is —CH₂C≡CCH₃. In some embodiments, R⁷ is optionally substituted C₁-C₃ alkyl. In some embodiments, R⁷ is C₁-C₃ alkyl. In some embodiments, R₈ is optionally substituted C₁-C₃ alkyl. In some embodiments, R⁸ is C₁-C₃ alkyl.

In some embodiments, the RAS(ON) inhibitor has the structure of Formula BIf, or a pharmaceutically acceptable salt thereof:

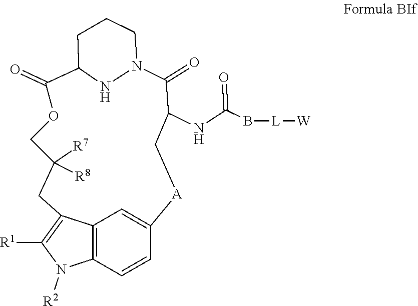

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, R¹ is optionally substituted 6 to 10-membered aryl, optionally substituted 3 to 6-membered cycloalkenyl, or optionally substituted 5 to 10-membered heteroaryl. In some embodiments, R¹ is optionally substituted 6-membered aryl, optionally substituted 6-membered cycloalkenyl, or optionally substituted 6-membered heteroaryl.

In some embodiments of Formula BI and subformula thereof, R₁ is

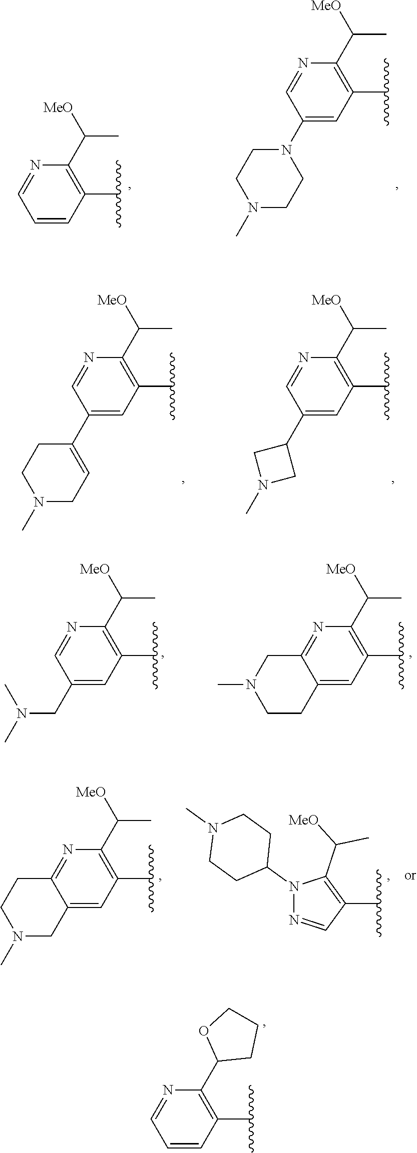

or a stereoisomer (e.g., atropisomer) thereof.

In some embodiments of Formula BI and subformula thereof, R₁ is

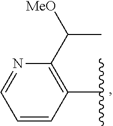 [SMILES: *c1cccnc1C(C)OC]

or a stereoisomer (e.g., atropisomer) thereof.

In some embodiments of Formula BI and subformula thereof, R₁ is

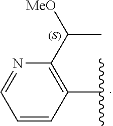

In some embodiments, the RAS(ON) inhibitor has the structure of Formula BIg, or a pharmaceutically acceptable salt thereof:

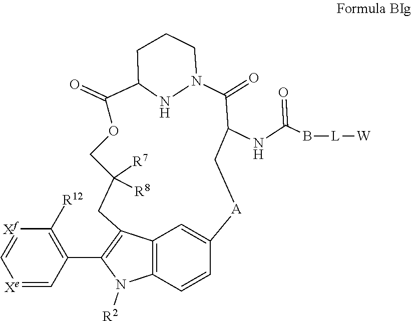

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} and X^{f} are, independently, N or CH; and
R¹² is optionally substituted C₁-C₆ alkyl, optionally substituted C₁-C₆ heteroalkyl, or optionally substituted 3 to 6-membered heterocycloalkylene.

In some embodiments of Formula BI and subformula thereof, X^{e} is N and X^{f} is CH. In some embodiments, X^{e} is CH and X^{f} is N.

In some embodiments of Formula BI and subformula thereof, R¹² is optionally substituted C₁-C₆ heteroalkyl.

In some embodiments, R¹² is

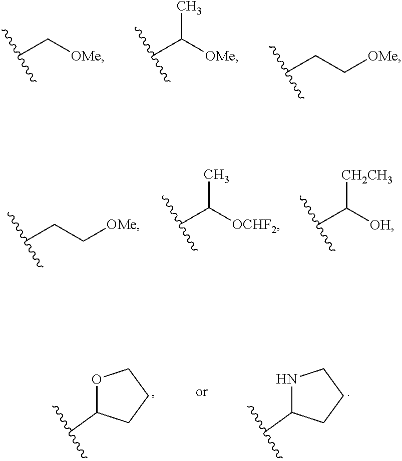

In some embodiments, R¹² is

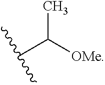 [SMILES: *C(C)OC]

In some embodiments, the RAS(ON) inhibitor has the structure of Formula BVI, or a pharmaceutically acceptable salt thereof:

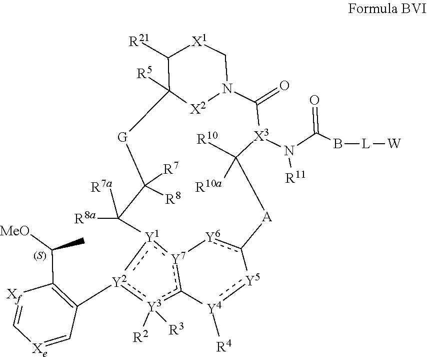

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C=CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R)—, —C(O)NH—CH(R⁶)— where C is bound to —C(R⁷R)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R²¹ is hydrogen or C₁-C₃ alkyl (e.g., methyl); and
X^{e} and X^{f} are, independently, N or CH.

In some embodiments, the RAS(ON) inhibitor has the structure of Formula BVIa, or a pharmaceutically acceptable salt thereof:

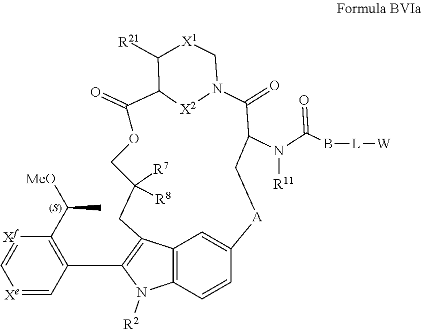

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)n;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of Formula BI and subformula thereof, X is N and X^{f} is CH. In some embodiments, X is CH and X^{f} is N.

In some embodiments, the RAS(ON) inhibitor has the structure of Formula BVIb, or a pharmaceutically acceptable salt thereof:

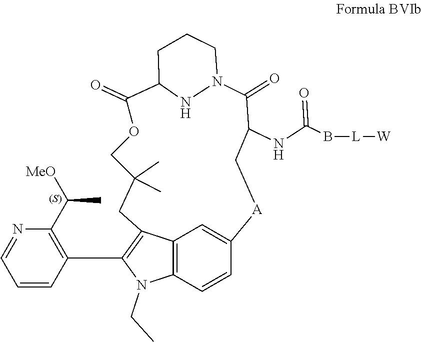

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
L is absent or a linker; and
W is a cross-linking group comprising a vinyl ketone, a vinyl sulfone, an ynone, or an alkynyl sulfone.

In some embodiments of formula BI or subformula thereof, A is optionally substituted 6-membered arylene.

In some embodiments, the RAS(ON) inhibitor has the structure of Formula BVIc, or a pharmaceutically acceptable salt thereof:

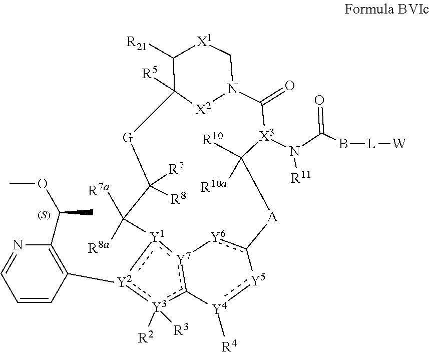

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R^{9′}, or >CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R)—, —C(O)NH—CH(R⁶)— where C is bound to —C(R⁷R)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)²R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R^{9′} is hydrogen or optionally substituted C₁-C₆ alkyl; or
R⁹ and R^{9′}, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI and subformula thereof, A has the structure:

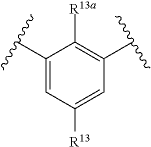

wherein R¹³ is hydrogen, halo, hydroxy, amino, optionally substituted C₁-C₆ alkyl, or optionally substituted C₁-C₆ heteroalkyl; and R^{13a} is hydrogen or halo. In some embodiments, R¹³ is hydrogen. In some embodiments, R¹³ and R^{13a} are each hydrogen. In some embodiments, R¹³ is hydroxy, methyl, fluoro, or difluoromethyl.

In some embodiments of Formula BI and subformula thereof, A is optionally substituted 5 to 6-membered heteroarylene.

In some embodiments, A is:

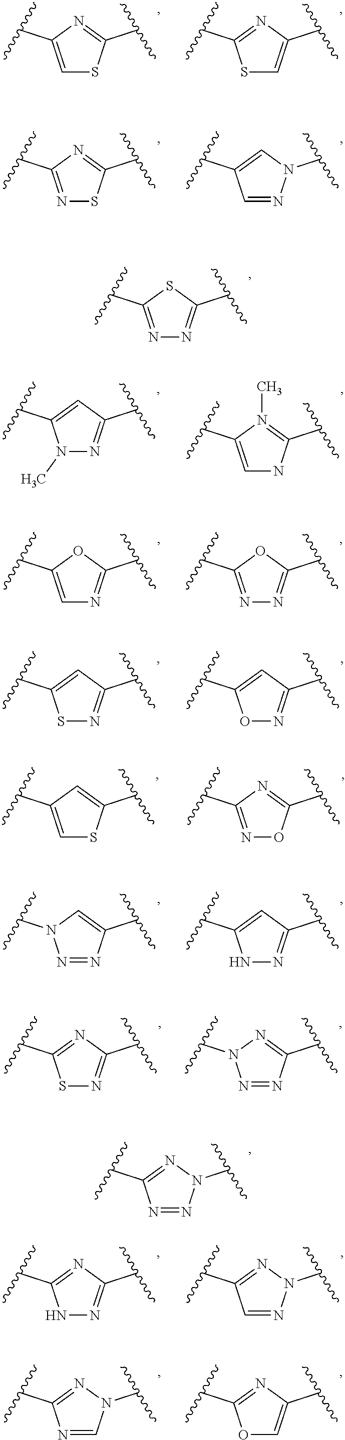

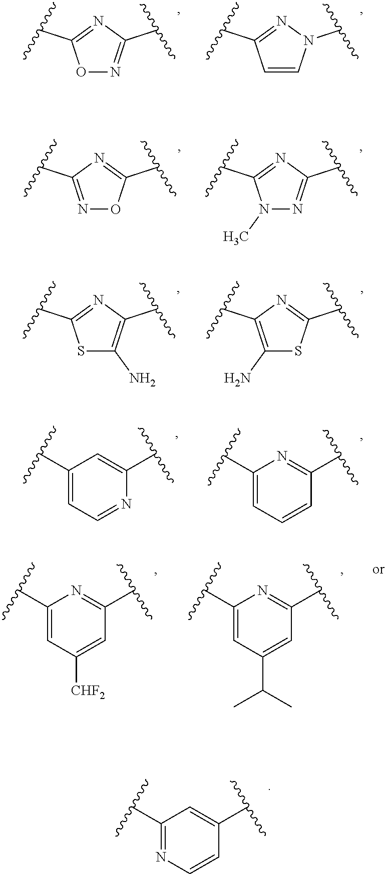

In some embodiments of Formula BI and subformula thereof, A is optionally substituted C₁-C₄ heteroalkylene.

In some embodiments, A is:

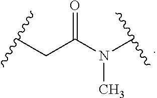 [SMILES: *CC(=O)N(*)C]

In some embodiments of Formula BI and subformula thereof, A is optionally substituted 3 to 6-membered heterocycloalkylene.

In some embodiments, A is:

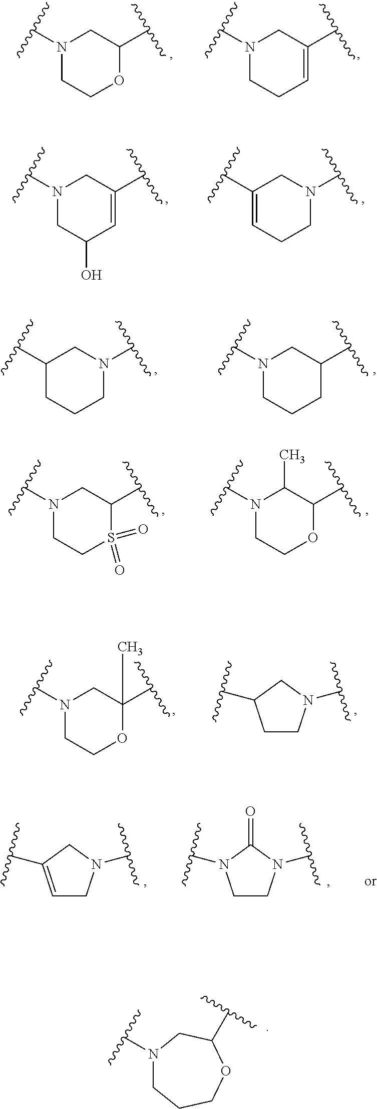

In some embodiments, A is

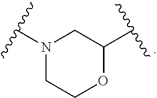 [SMILES: *C1CN(*)CCO1]

In some embodiments of Formula BI and subformula thereof, B is —CHR⁹—. In some embodiments of Formula BI and subformula thereof, R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl. In some embodiments, R⁹ is:

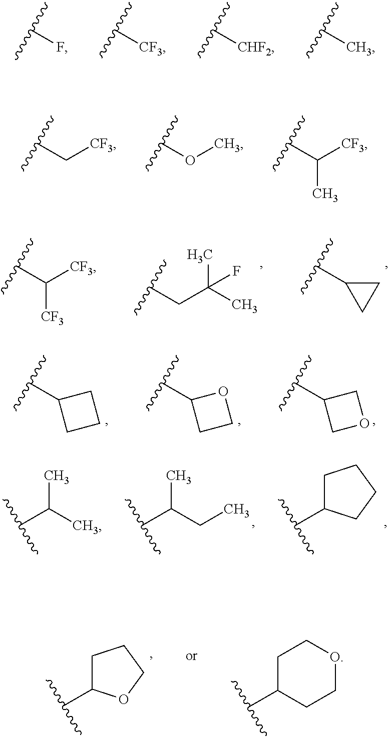

In some embodiments, R⁹ is:

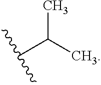 [SMILES: *C(C)C]

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, B is optionally substituted 6-membered arylene. In some embodiments, B is 6-membered arylene. In some embodiments, B is:

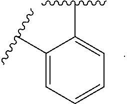 [SMILES: *c1ccccc1*]

In some embodiments of Formula BI and subformula thereof, R⁷ is methyl.

In some embodiments of Formula BI and subformula thereof, R⁸ is methyl.

In some embodiments of Formula BI and subformula thereof, R²¹ is hydrogen.

In some embodiments of Formula BI and subformula thereof, the linker is the structure of Formula BII:

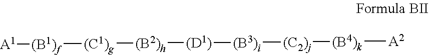

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹—(B¹)_{f}—(C¹)_{g}—(B²)ₕ— to —(B³)ᵢ—(C²)ⱼ—(B⁴)ₖ-A². In some embodiments, the linker is acyclic.

In some embodiments, linker has the structure of Formula BIIa:

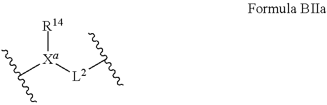

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

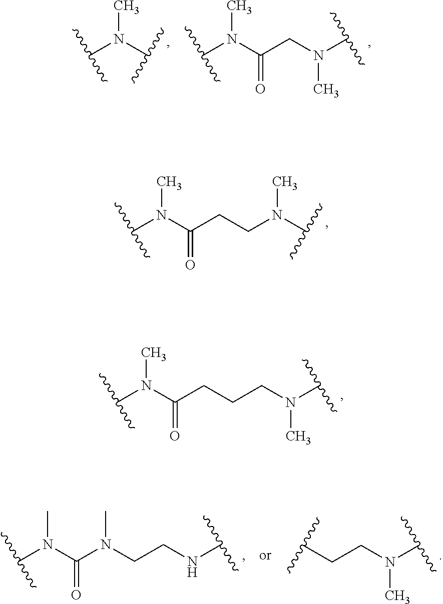

In some embodiments of Formula BI and subformula thereof, the linker is or comprises a cyclic moiety. In some embodiments, the linker has the structure of Formula BIIb:

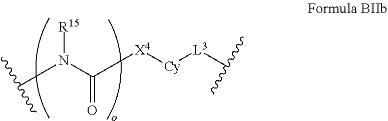

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
X⁴ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C1-C₄ alkylene or optionally substituted C₁-C₄ heteroalkylene.

In some embodiments of Formula BI and subformula thereof, the linker has the structure of Formula BIIb-1:

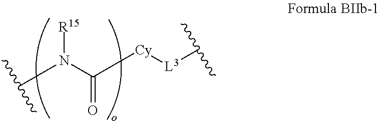

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments of Formula Bland subformula thereof, the linker has the structure of Formula BIIc:

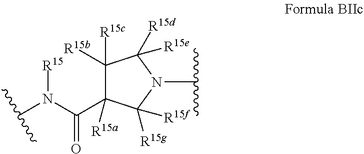

wherein R¹⁵ is hydrogen, optionally substituted C₁-C₆ alkyl, optionally substituted 3 to 8-membered cycloalkylene, or optionally substituted 3 to 8-membered heterocycloalkylene; and
R^{15a}, R^{15b}, R^{15c}, R^{15d}, R^{15e}, R^{15f}, and R^{15g} are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.

In some embodiments of Formula BI and subformula thereof, the linker has the structure:

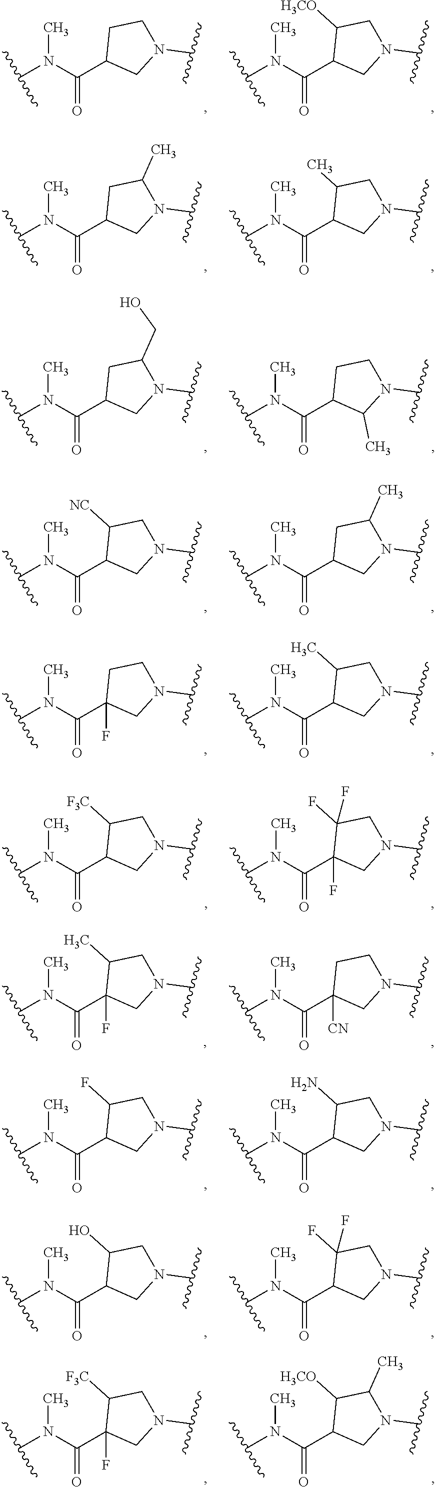

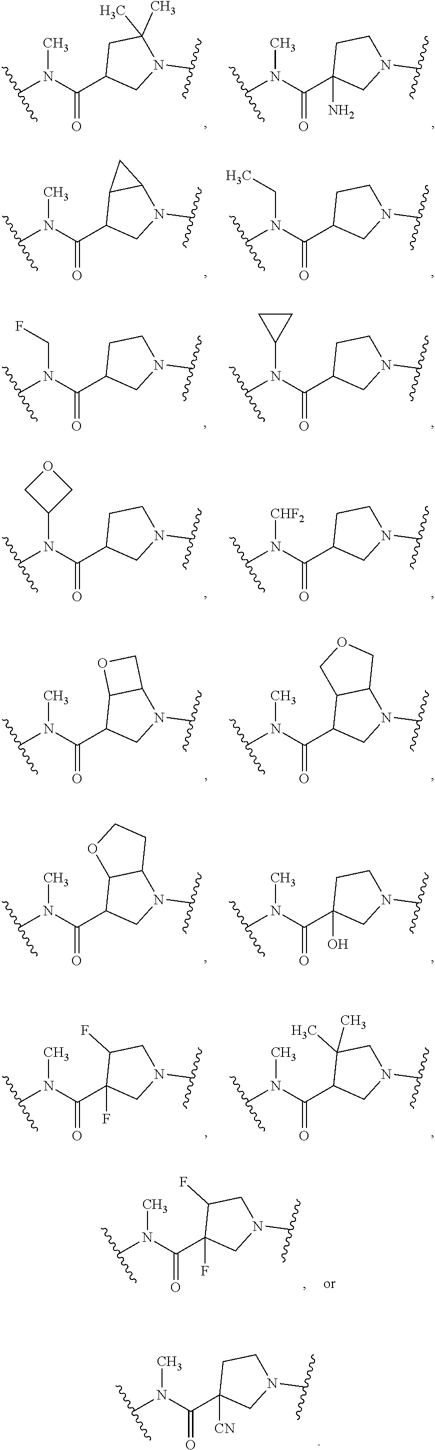

In some embodiments of Formula BI and subformula thereof, the linker has the structure:

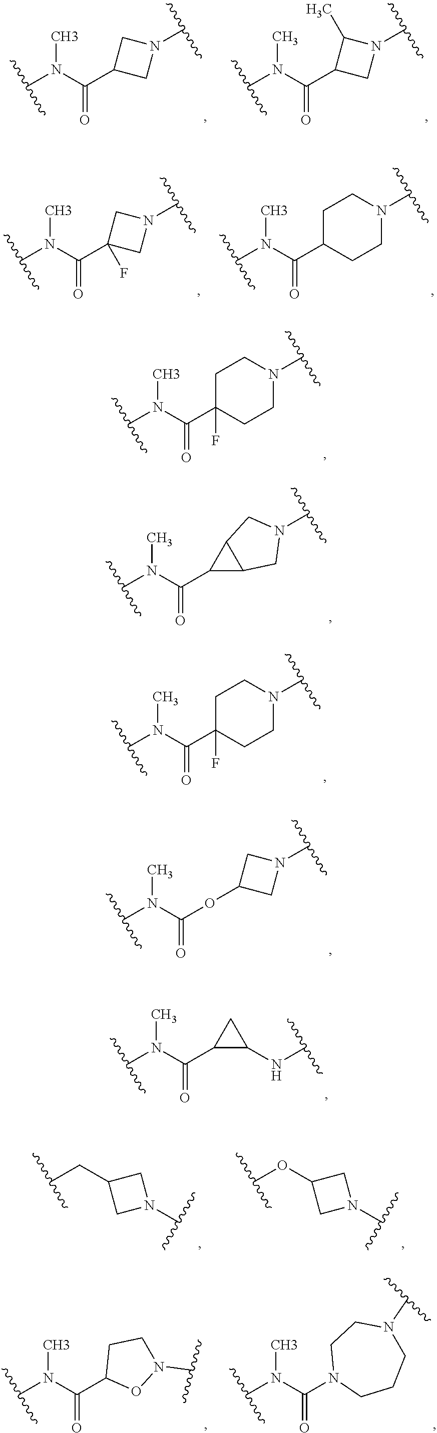

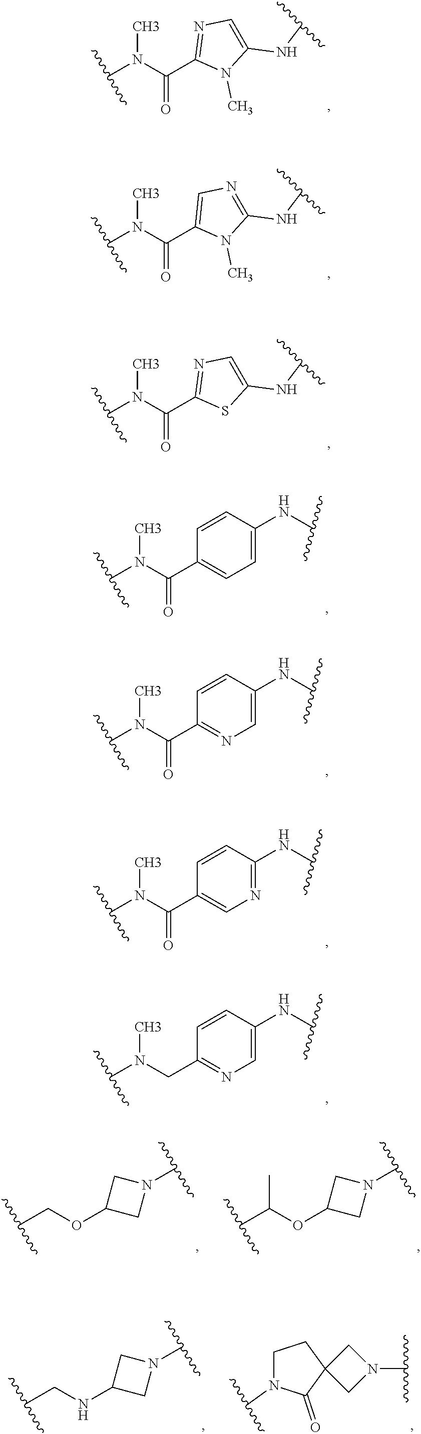

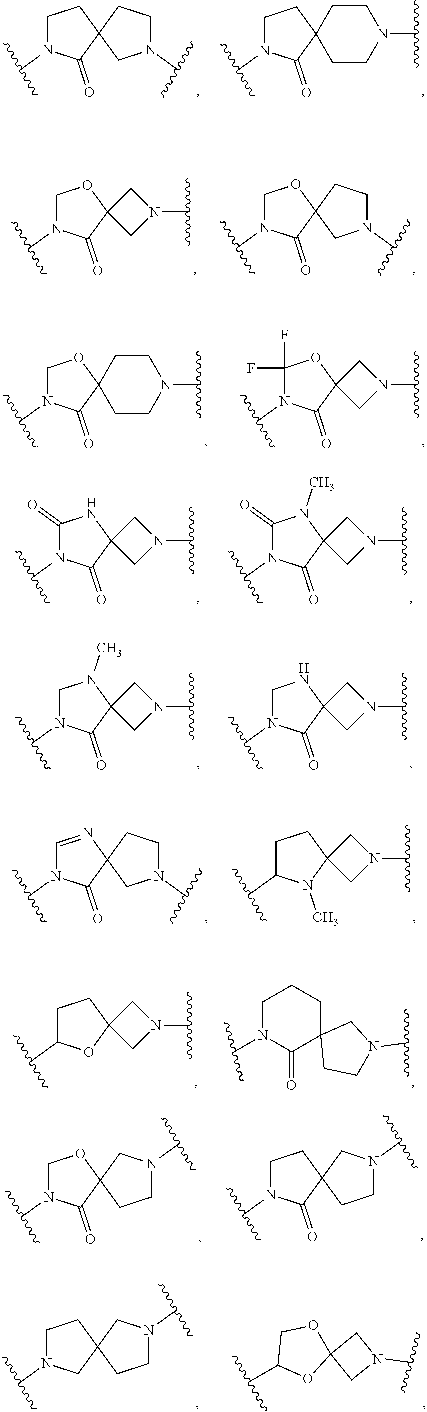

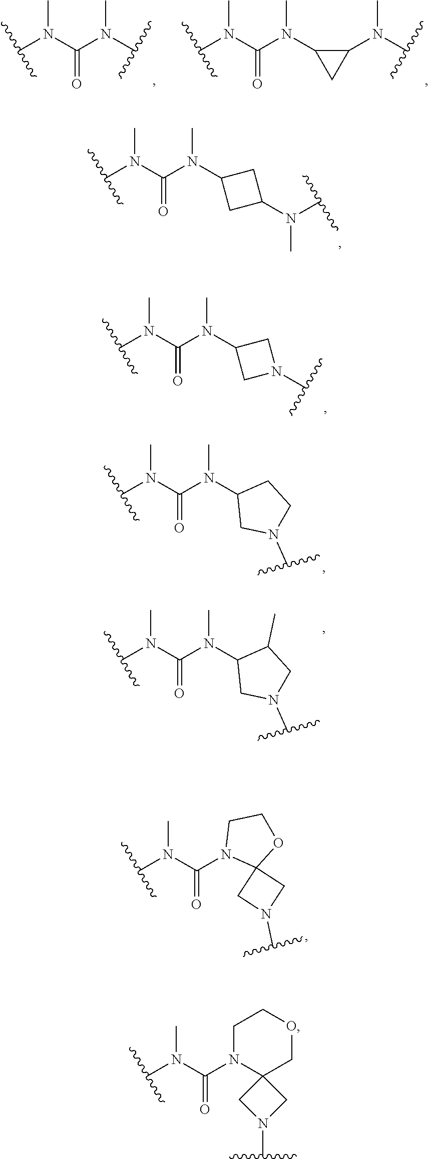

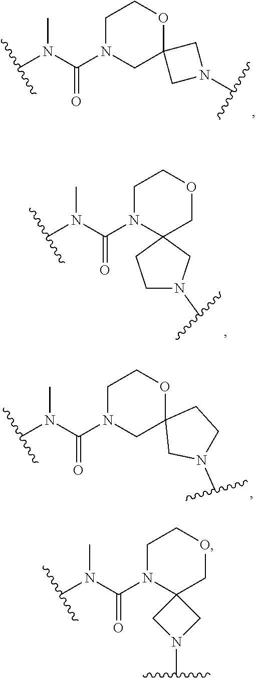 [SMILES: *N1CC2(C1)CN(C(=O)N(*)C)CCO2 ; *N1CCC2(COCCN2C(=O)N(*)C)C1 ; *N1CCC2(C1)CN(C(=O)N(*)C)CCO2 ; *N1CC2(COCCN2C(=O)N(*)C)C1]

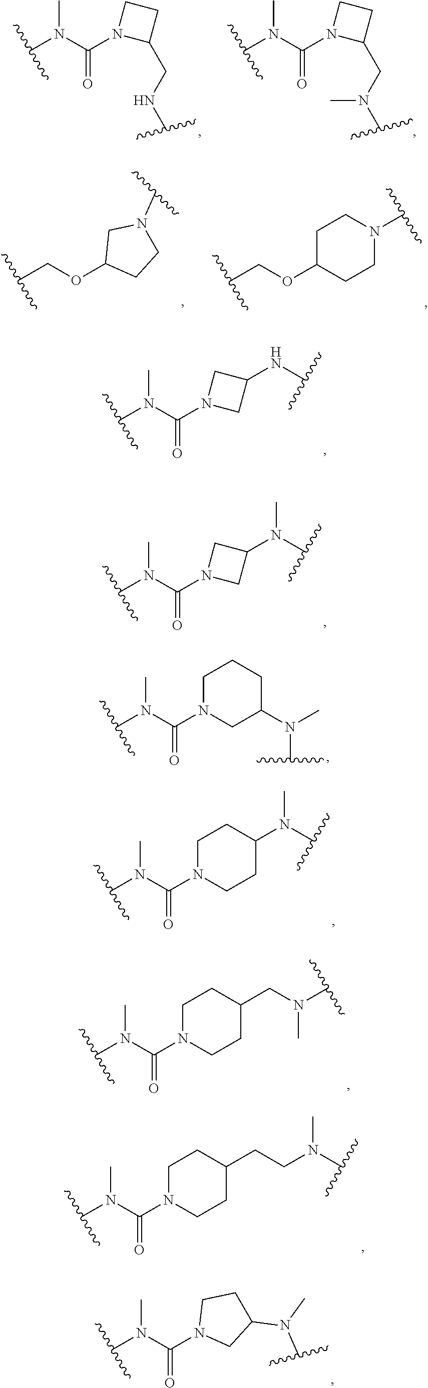

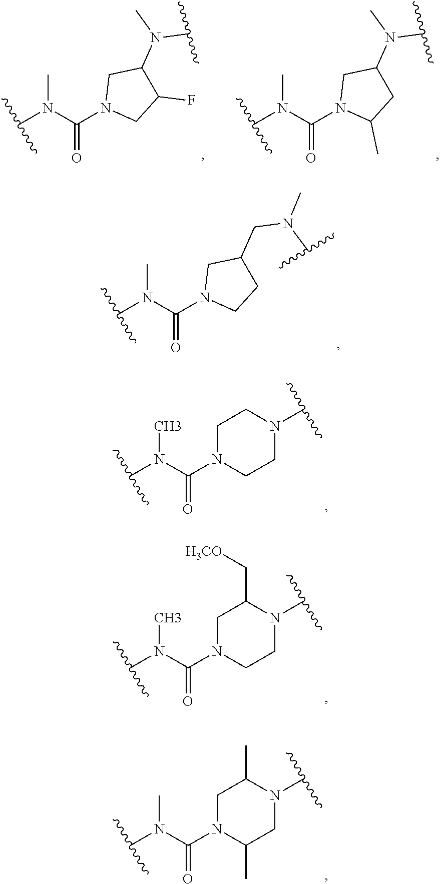

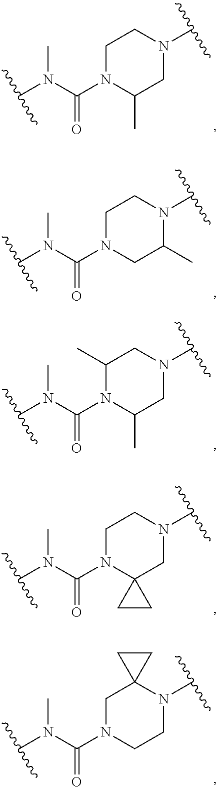

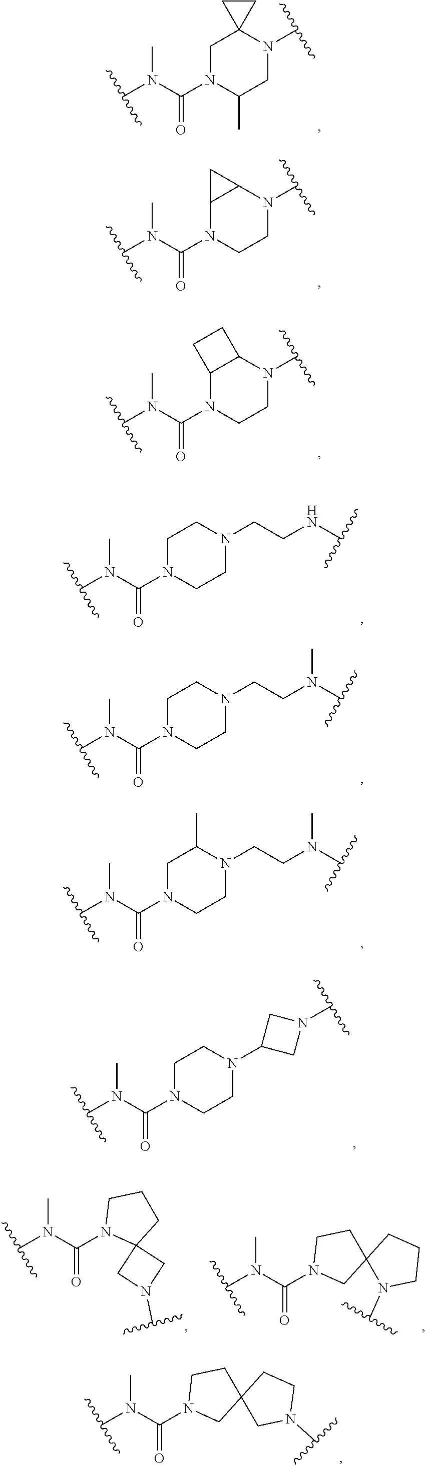

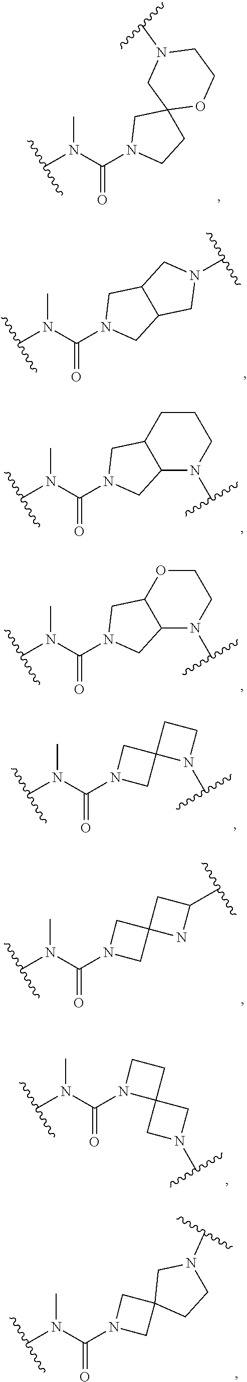

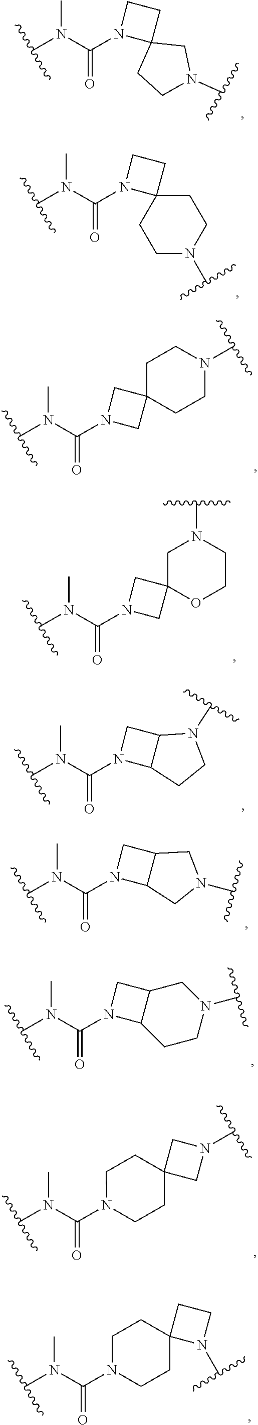

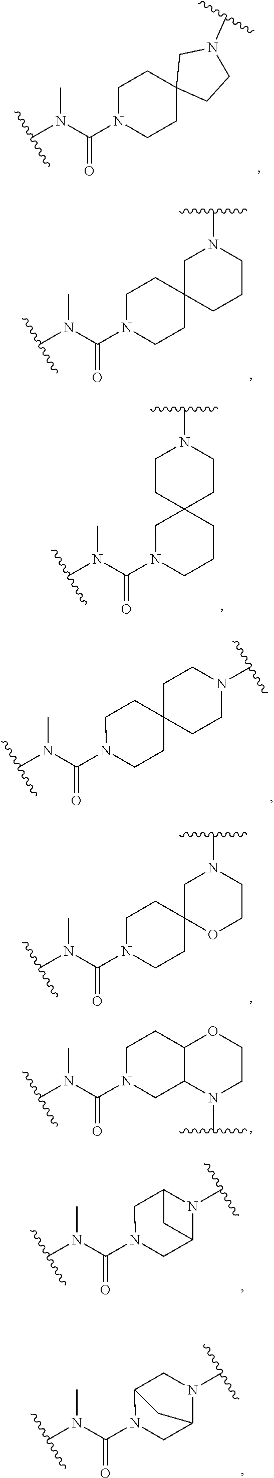

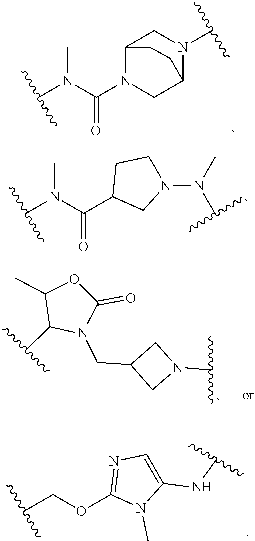

In some embodiments of Formula BI and subformula thereof, the linker has the structure

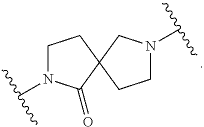 [SMILES: *N1CCC2(CCN(*)C2=O)C1]

In some embodiments of Formula BI and subformula thereof, the linker has the structure

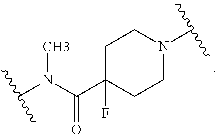 [SMILES: *N1CCC(F)(C(=O)N(*)C)CC1]

In some embodiments of Formula BI and subformula thereof, W is a cross-linking group comprising a vinyl ketone. In some embodiments, W has the structure of Formula BIIIa:

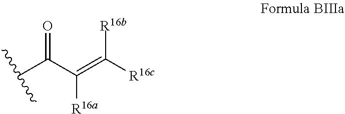

wherein R^{16a}, R^{16b}, and R^{16c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

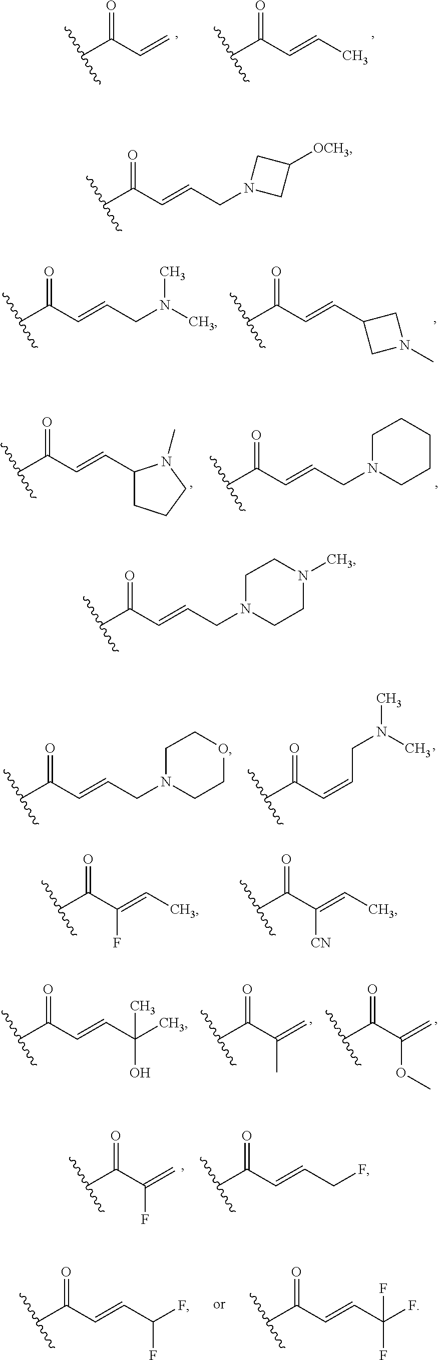

In some embodiments of Formula BI and subformula thereof, W is a cross-linking group comprising an ynone. In some embodiments, W has the structure of Formula BIIIb:

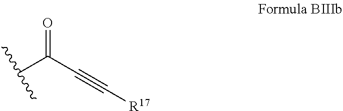

wherein R¹⁷ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl.

In some embodiments, W is:

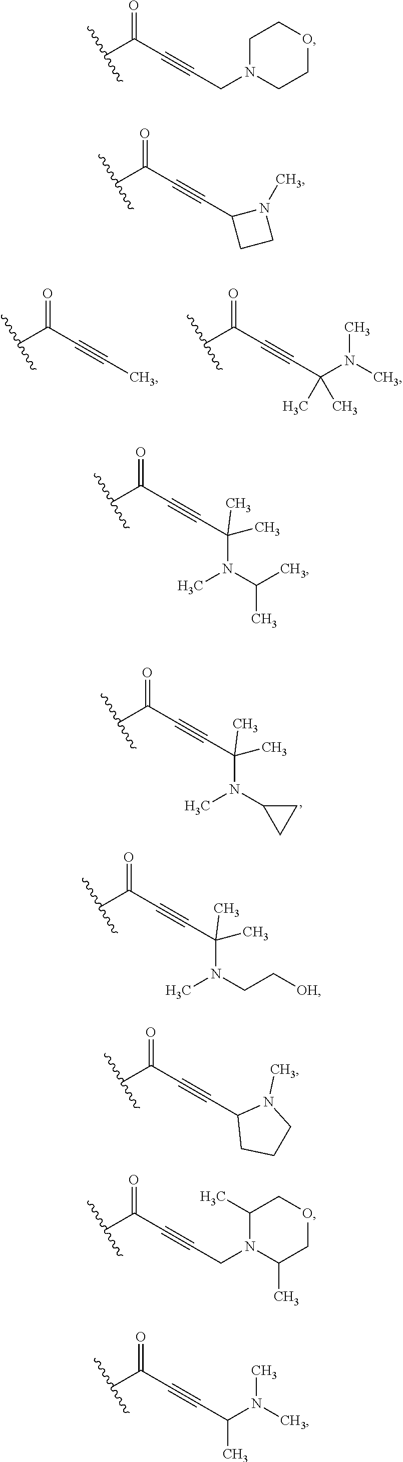

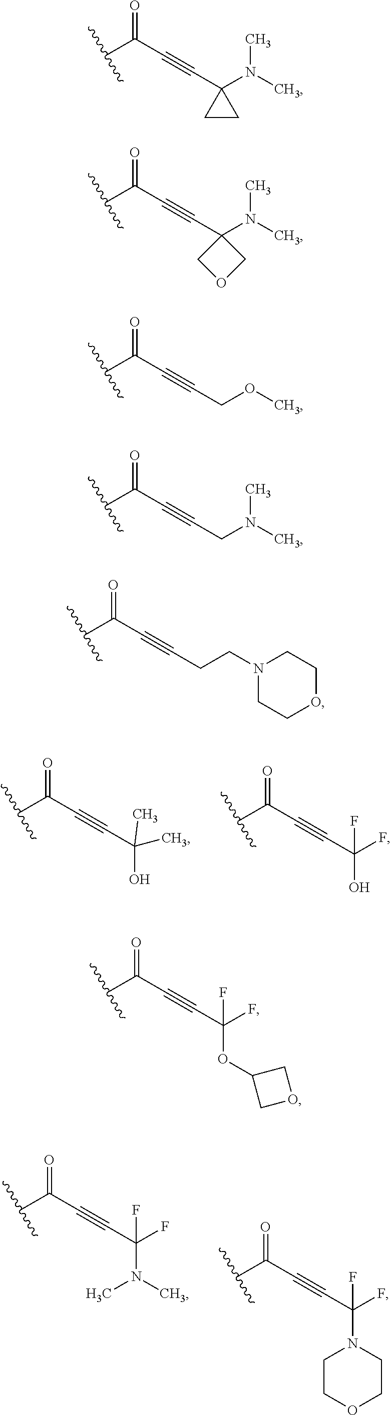

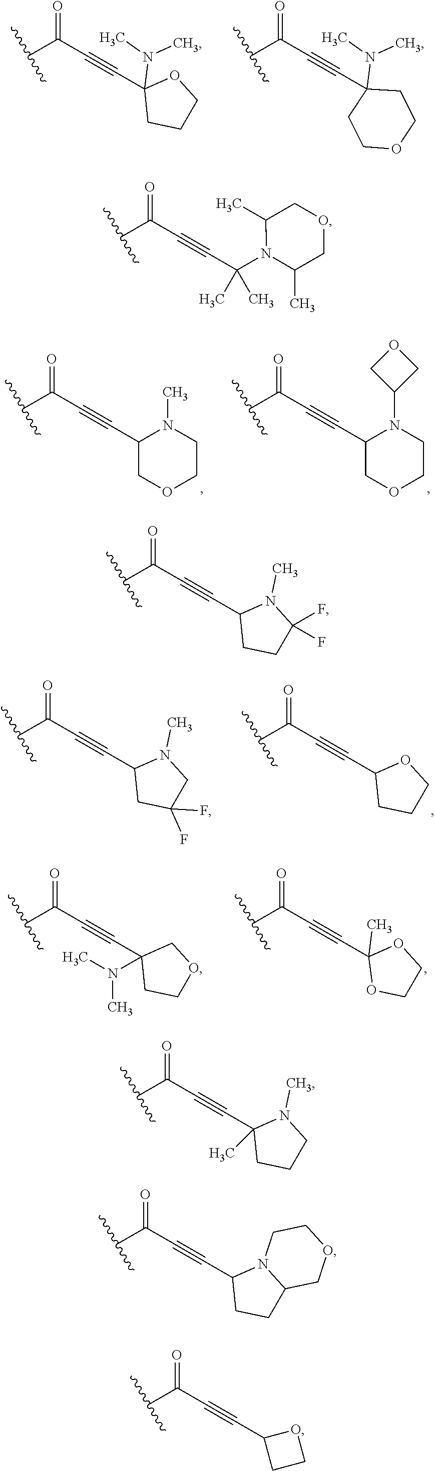

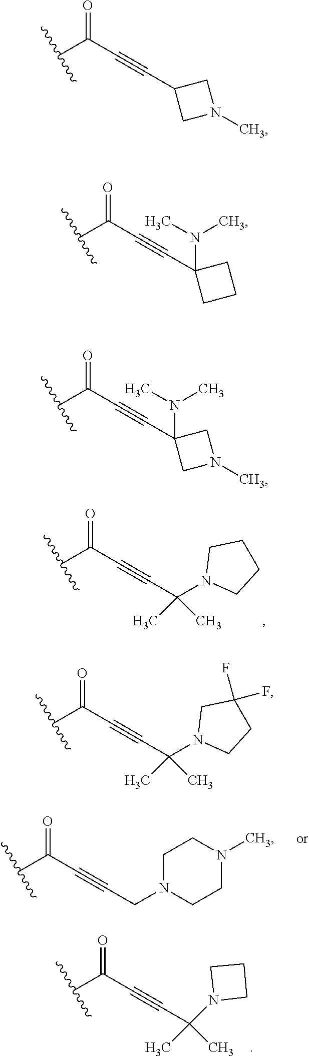

In some embodiments, W is

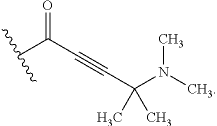 [SMILES: *C(=O)C#CC(C)(C)N(C)C]

In some embodiments of Formula BI and subformula thereof, W is a cross-linking group comprising a vinyl sulfone. In some embodiments, W has the structure of Formula BIIIc:

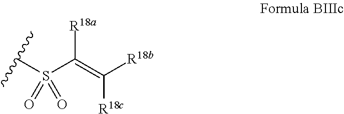

wherein R^{18a}, R^{18b}, and R^{18c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl.

In some embodiments, W is:

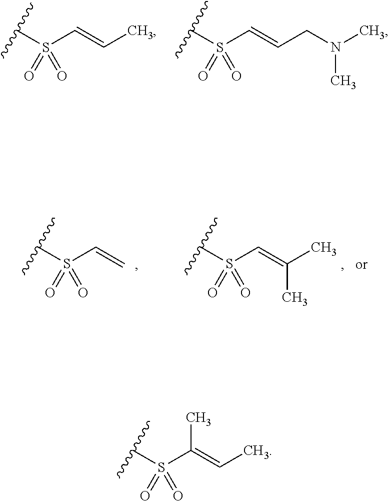

In some embodiments of Formula BI and subformula thereof, W is a cross-linking group comprising an alkynyl sulfone.

In some embodiments, W has the structure of Formula BIIId:

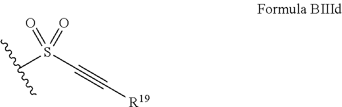

wherein R¹⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl.

In some embodiments, W is:

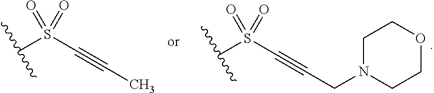

In some embodiments of Formula BI and subformula thereof, W has the structure of Formula BIIIe:

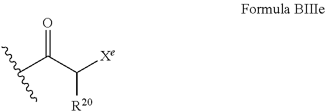

wherein X^{e} is a halogen; and
R²⁰ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments of Formula BI and subformula thereof, W is haloacetyl. In some embodiments of Formula BI and subformula thereof, W is not haloacetyl.

In some embodiments, the RAS(ON) inhibitor is selected from Table B1, or a pharmaceutically acceptable salt or stereoisomer thereof. In some embodiments, the RAS(ON) inhibitor is selected from Table B1, or a pharmaceutically acceptable salt or atropisomer thereof.

TABLE B1
Certain Compounds of the Present Invention

| Ex# | Structure |
|---|---|
| BA1 | 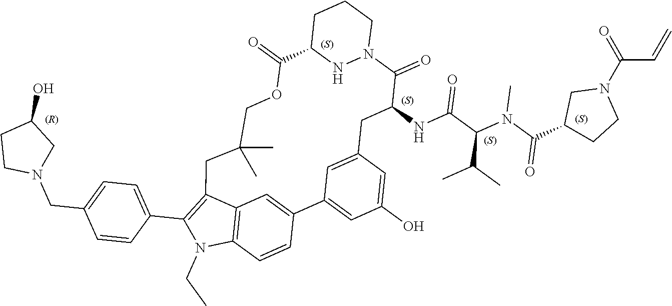 |
| BA2 | 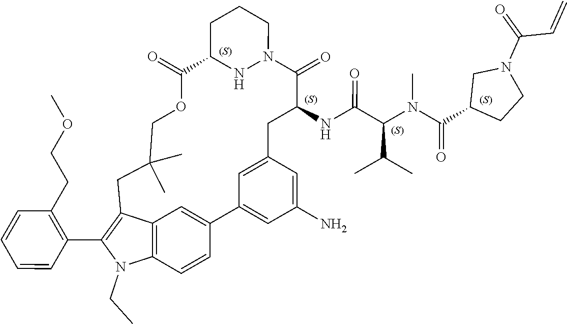 |
| BA3 | 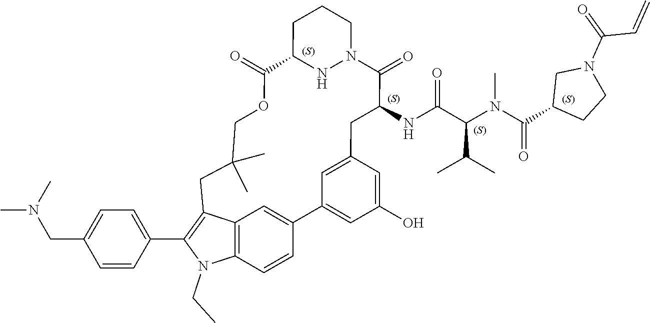 |
| BA4 | 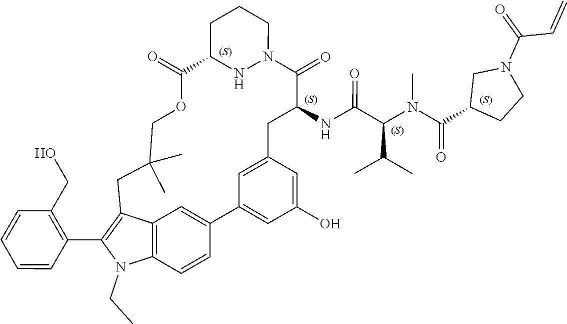 |
| BA5 | 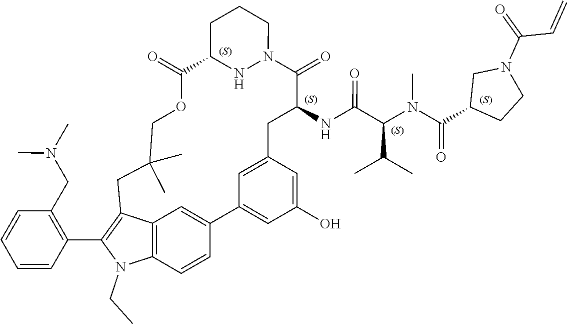 |
| BA6 | 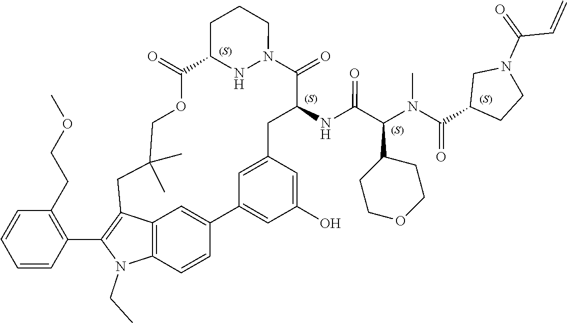 |
| BA7 | 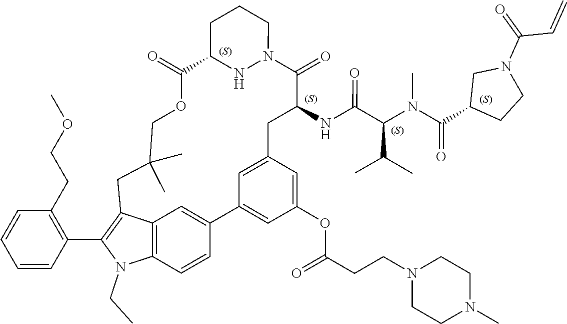 |
| BA8 | 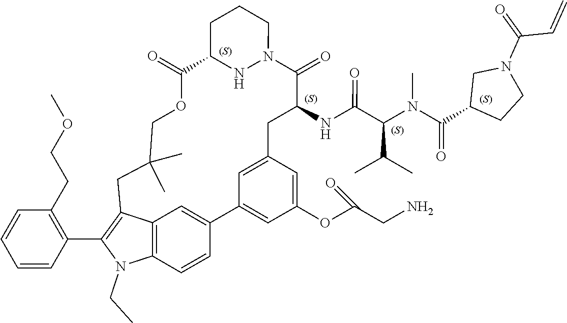 |
| BA9 | 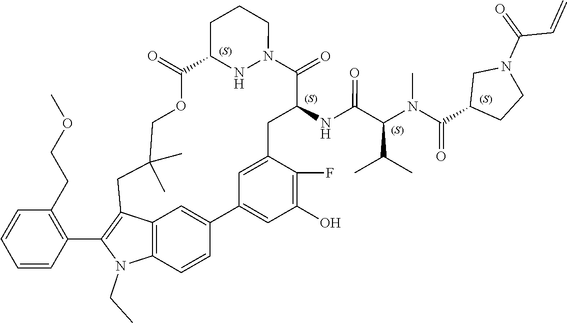 |
| BA10 | 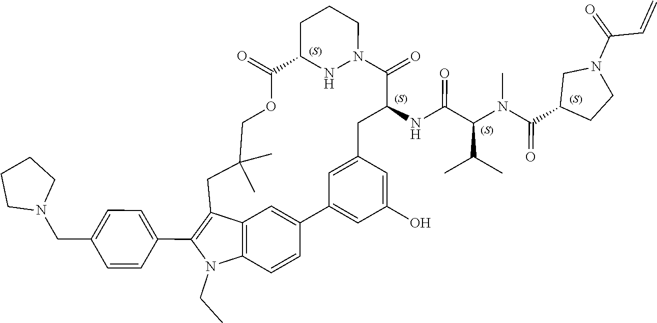 |
| BA11 | 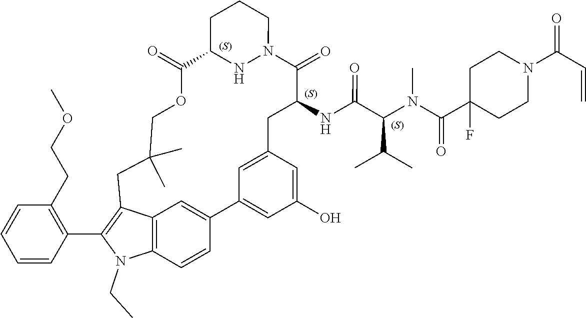 |
| BA12 | 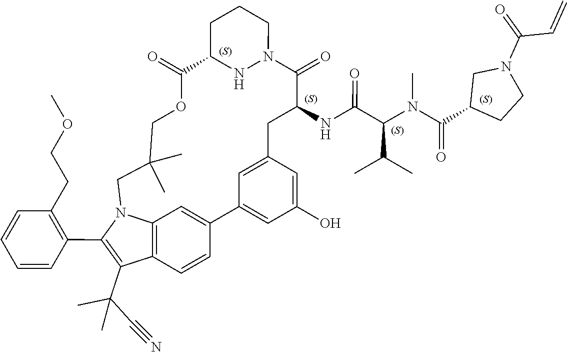 |
| BA13 | 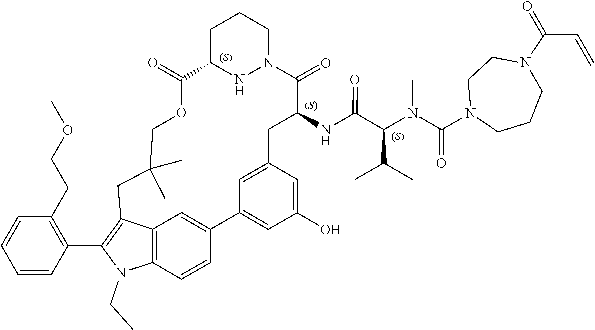 |
| BA14 | 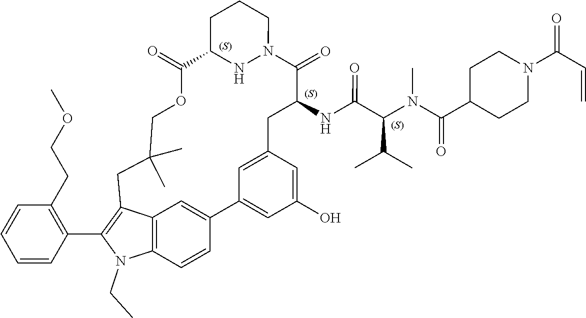 |
| BA15 | 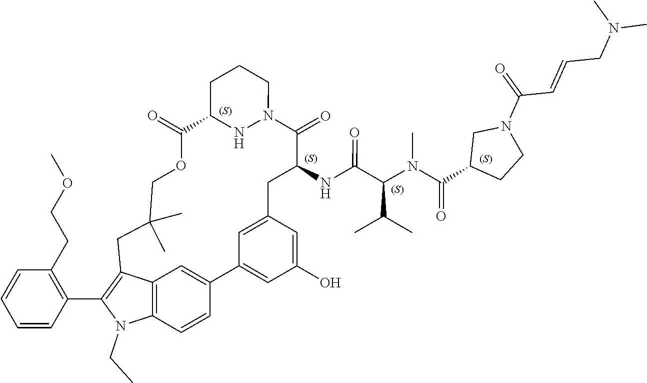 |
| BA16 | 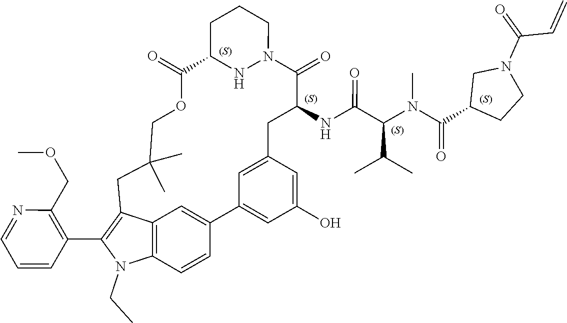 |
| BA17 | 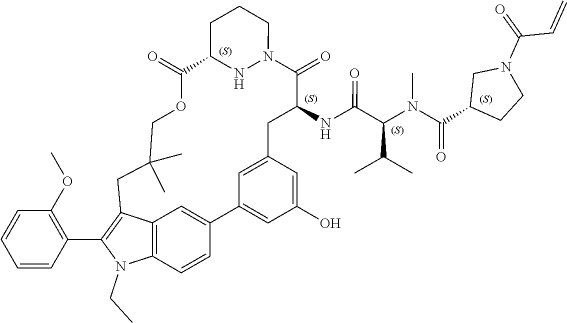 |
| BA18 | 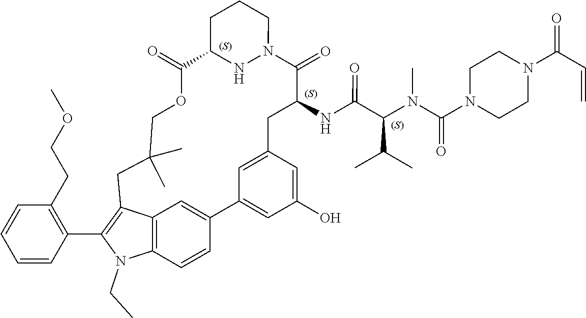 |
| BA19 | 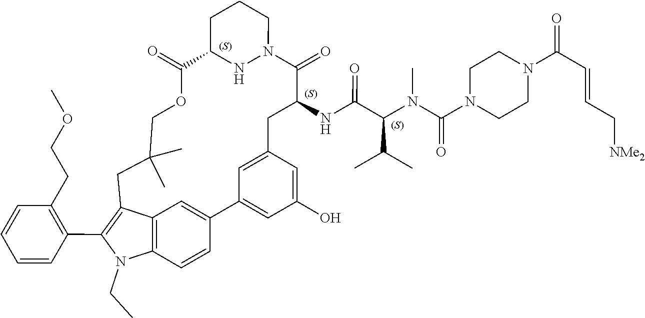 |
| BA20 | 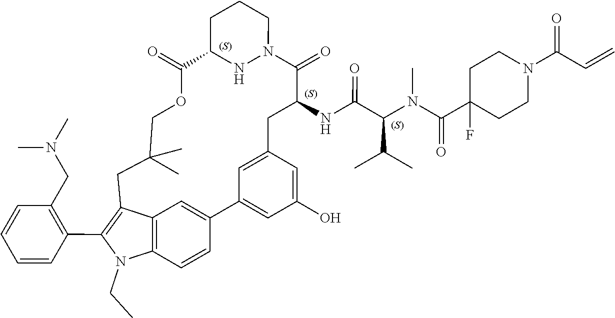 |
| BA21 | 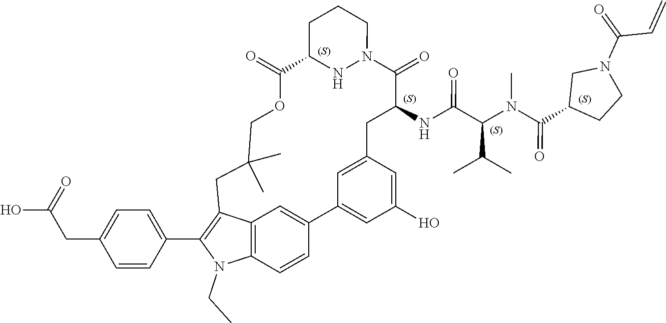 |
| BA22 | 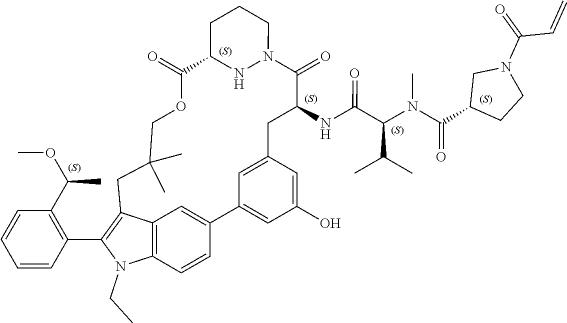 |
| BA23 | 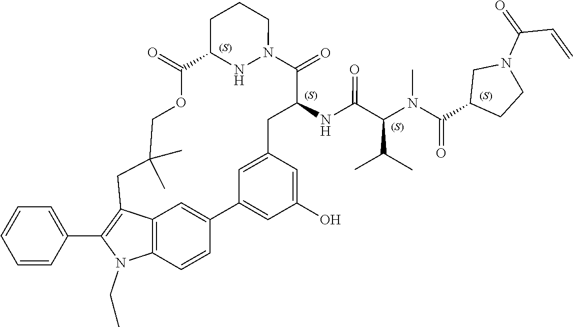 |
| BA24 | 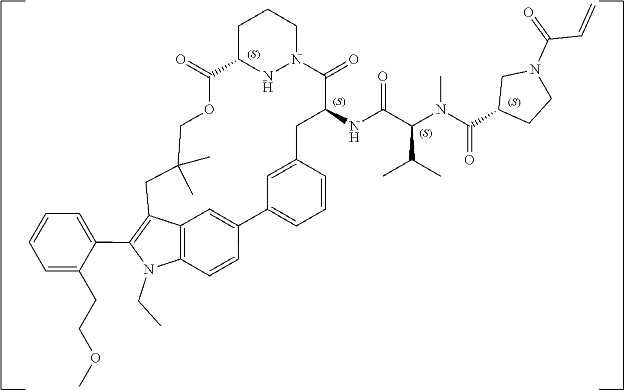 |
| BA25 | 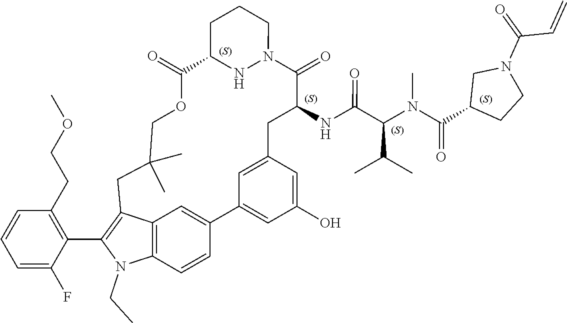 |
| BA26 | 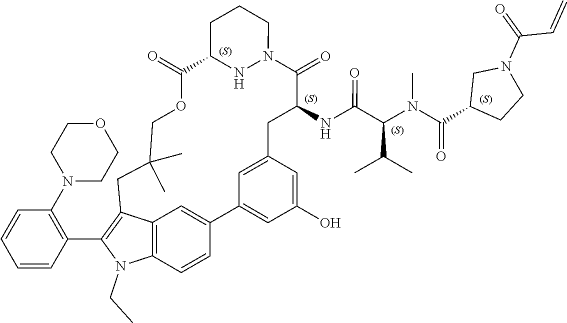 |
| BA27 | 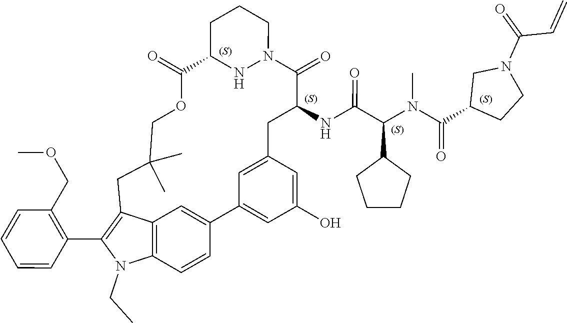 |
| BA28 | 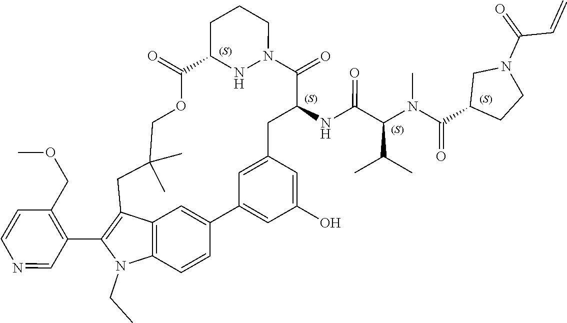 |
| BA29 | 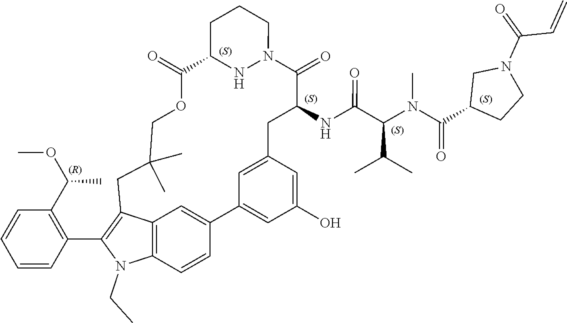 |
| BA30 | 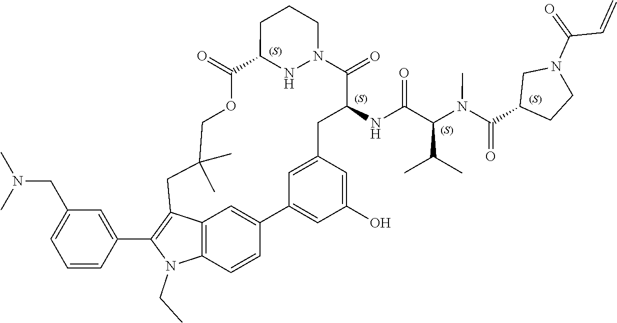 |
| BA31 | 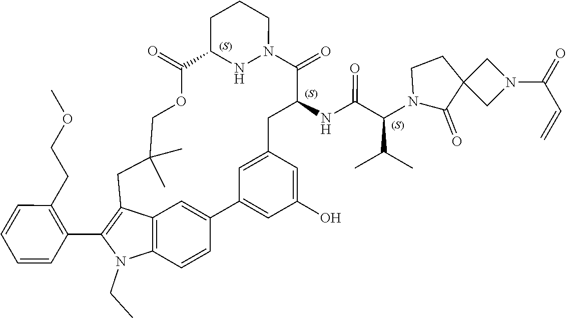 |
| BA32 | 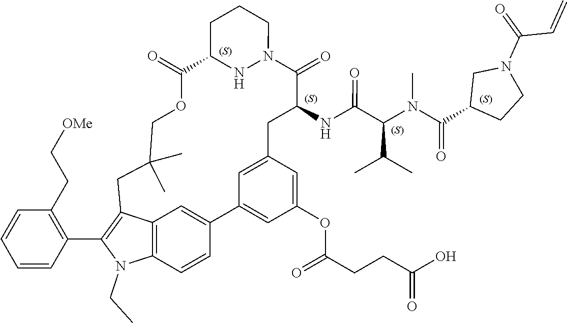 |
| BA33 | 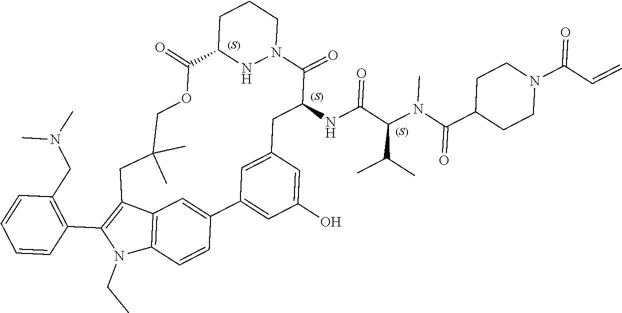 |
| BA34 | 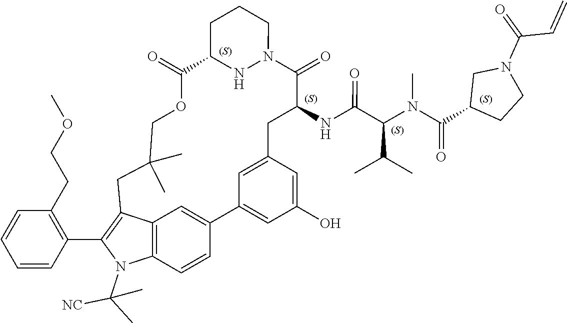 |
| BA35 | 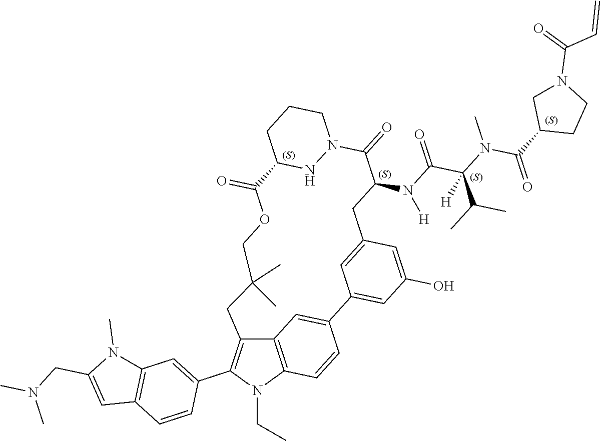 |
| BA36 | 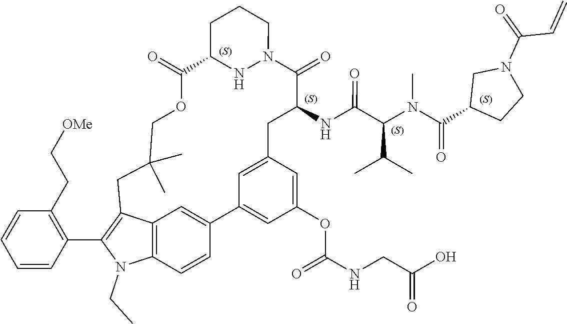 |
| BA37 | 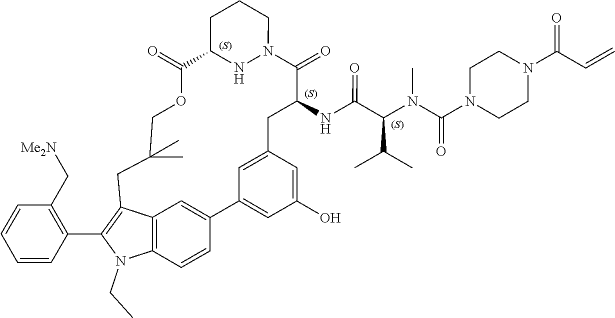 |
| BA38 | 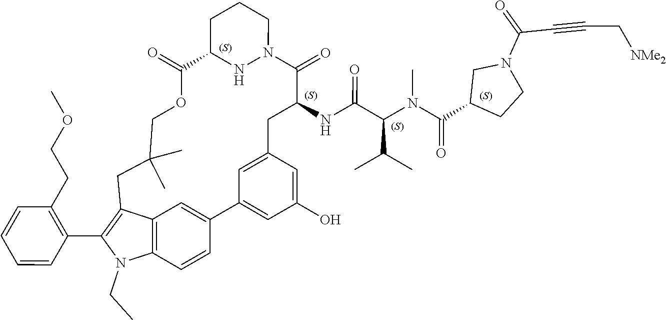 |
| BA39 | 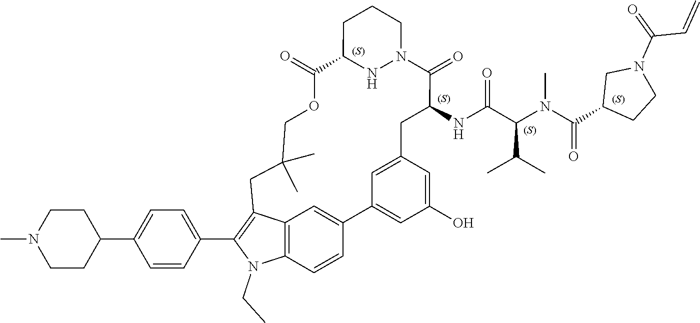 |
| BA40 | 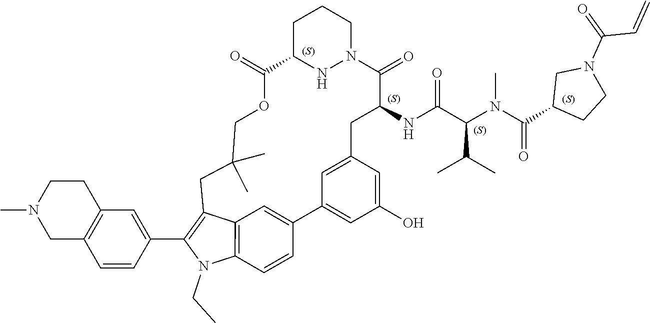 |
| BA41 | 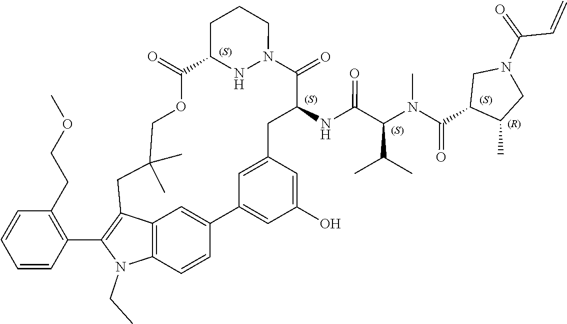 |
| BA42 | 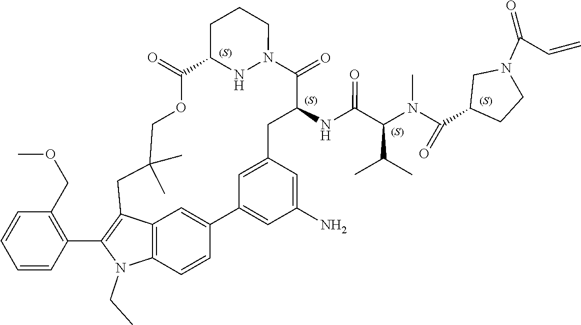 |
| BA43 | 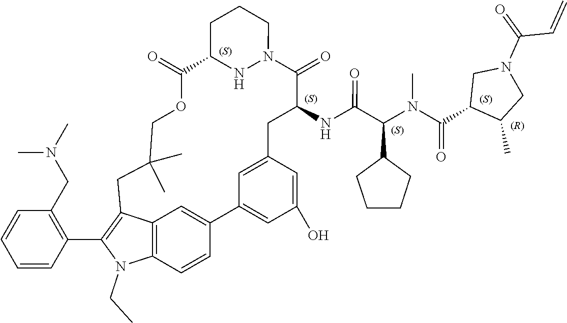 |
| BA44 | 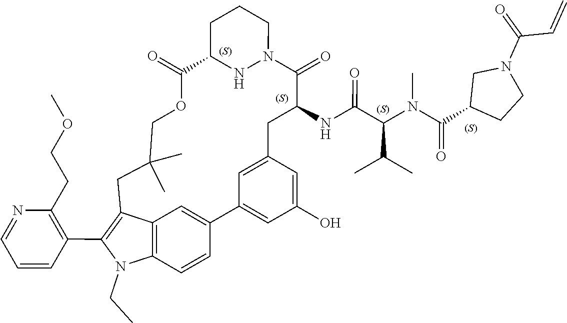 |
| BA45 | 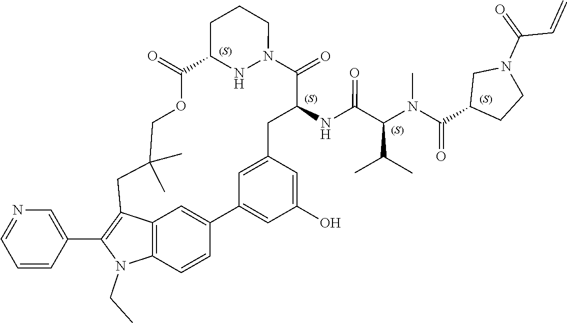 |
| BA46 | 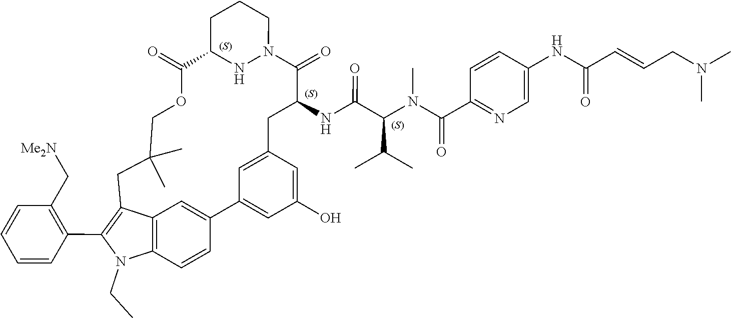 |
| BA47 | 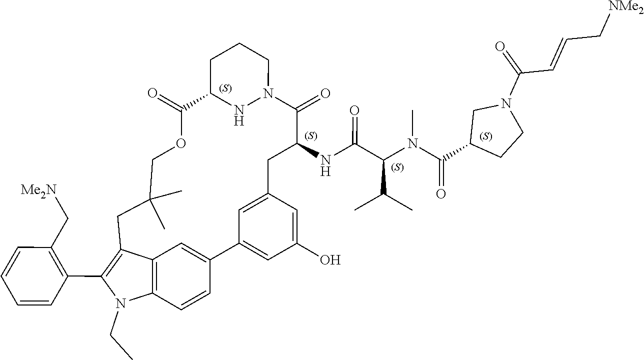 |
| BA48 | 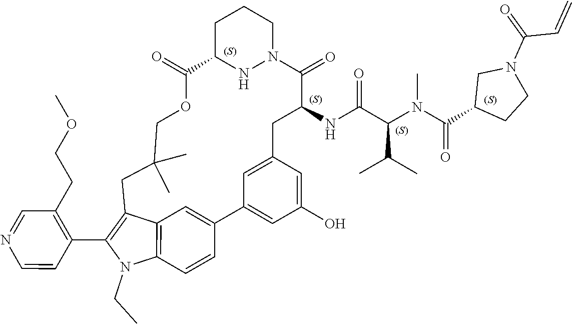 |
| BA49 | 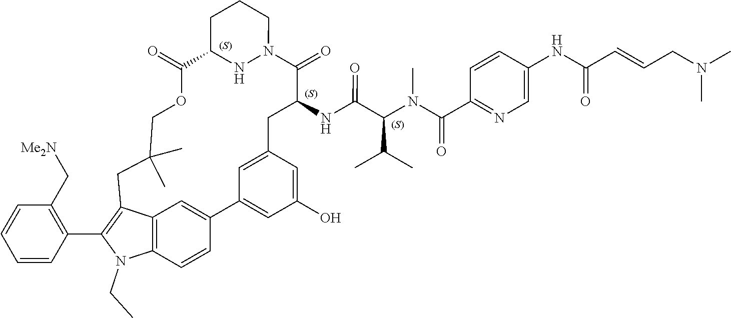 |
| BA50 | 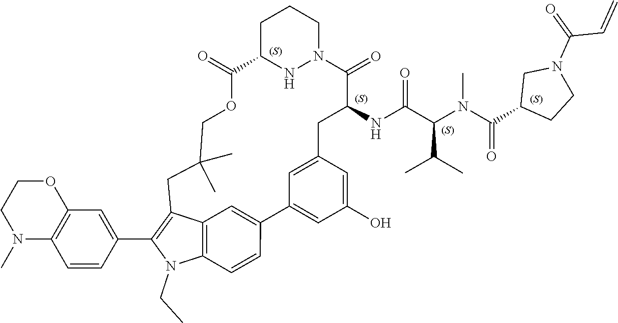 |
| BA51 | 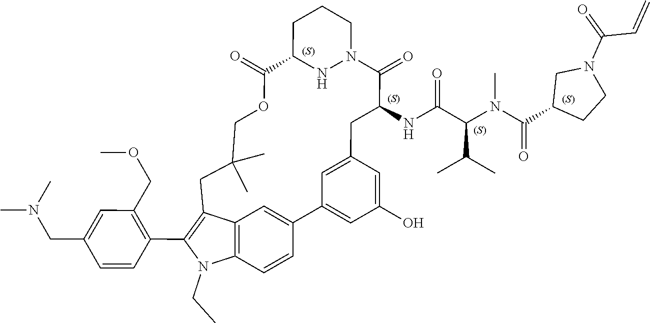 |
| BA52 | 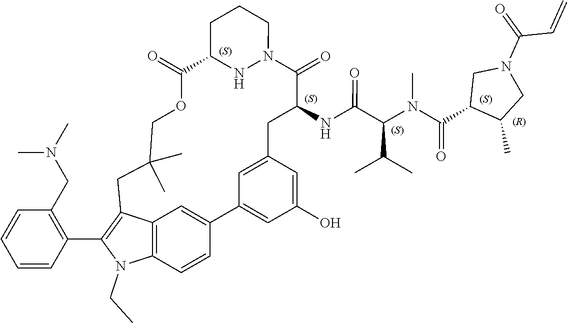 |
| BA53 | 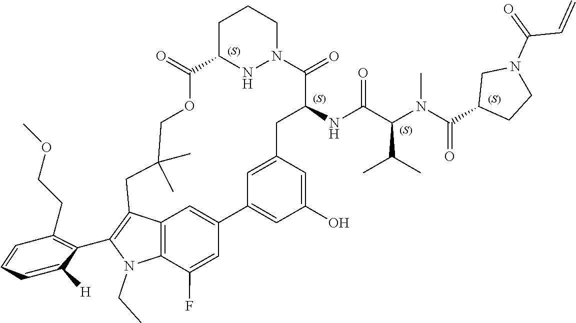 |
| BA54 | 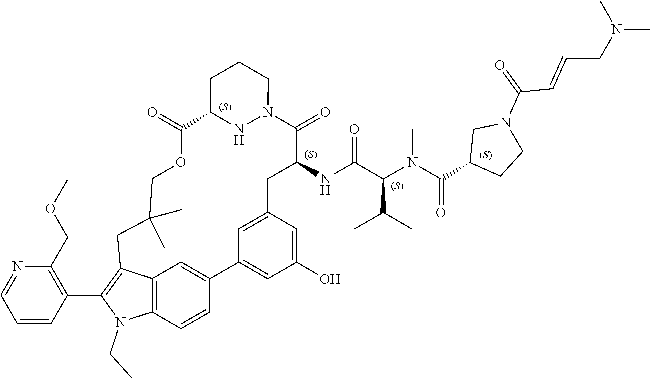 |
| BA55 | 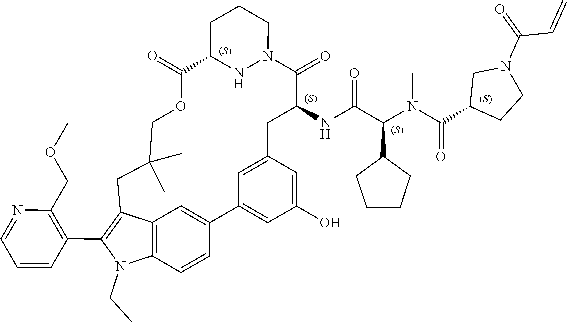 |
| BA56 | 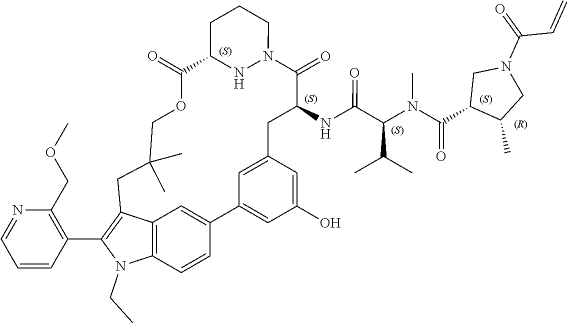 |
| BA57 | 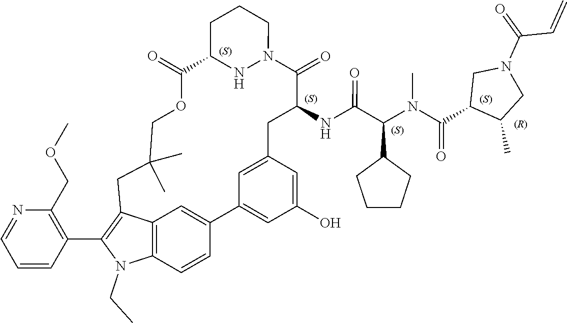 |
| BA58 | 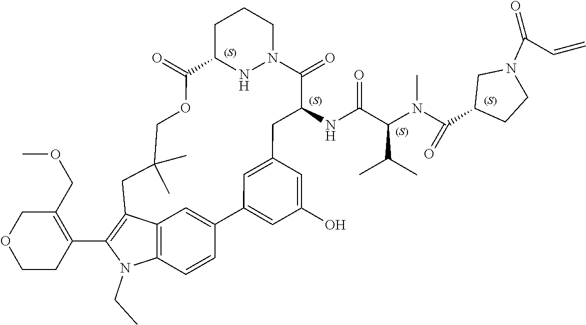 |
| BA59 | 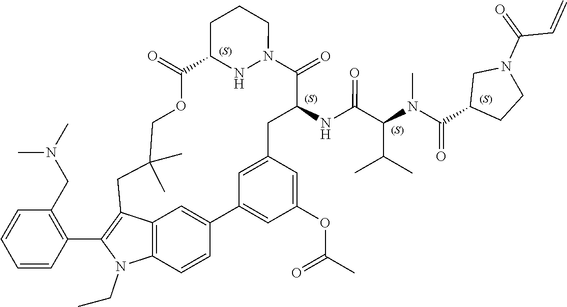 |
| BA60 | 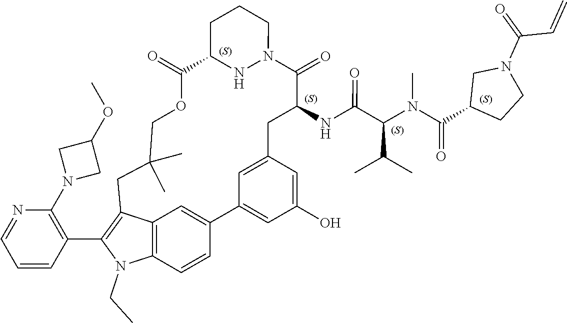 |
| BA61 | 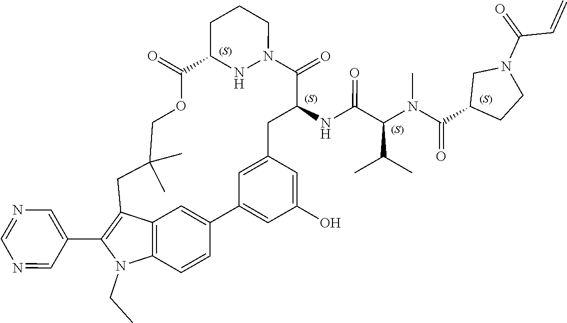 |
| BA62 | 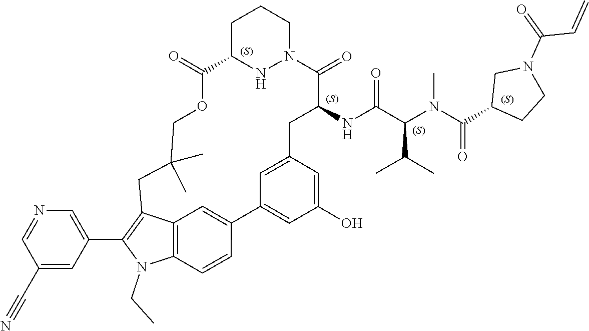 |
| BA63 | 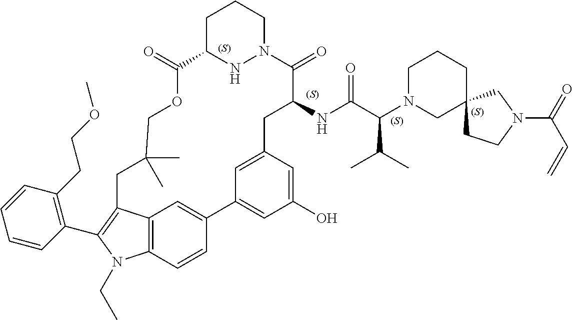 |
| BA64 | 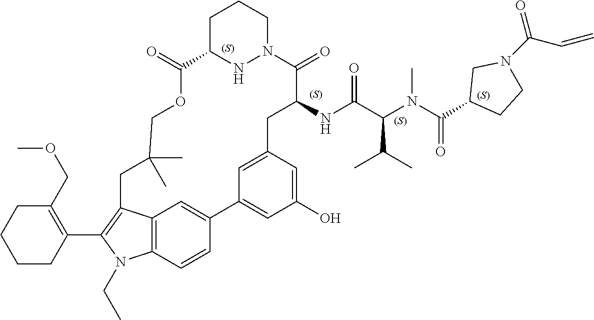 |
| BA65 | 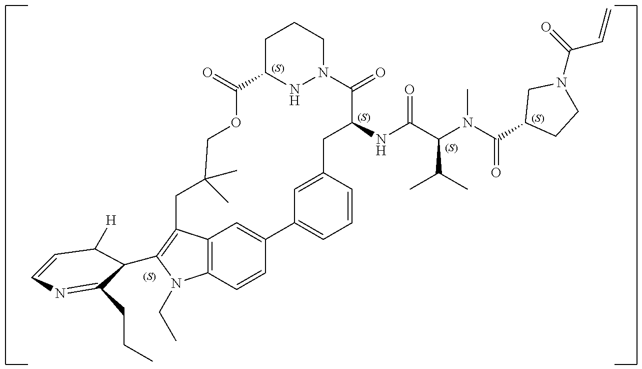 |
| BA66 | 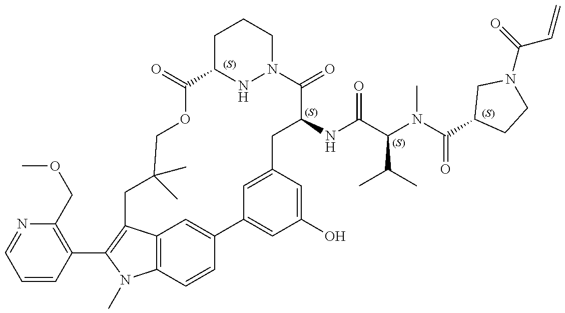 |
| BA67 | 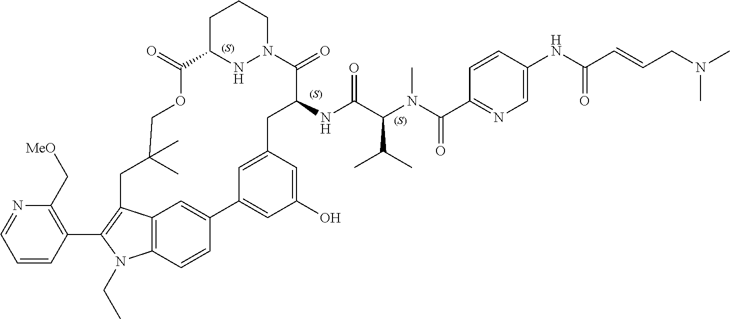 |
| BA68 | 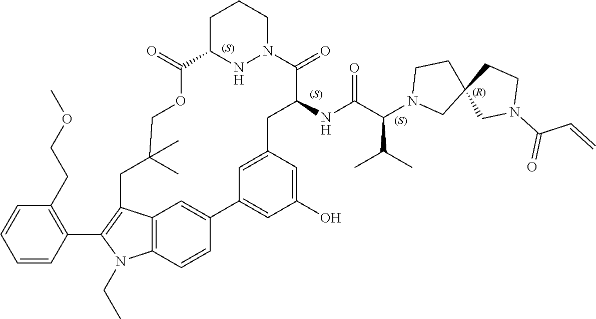 |
| BA69 | 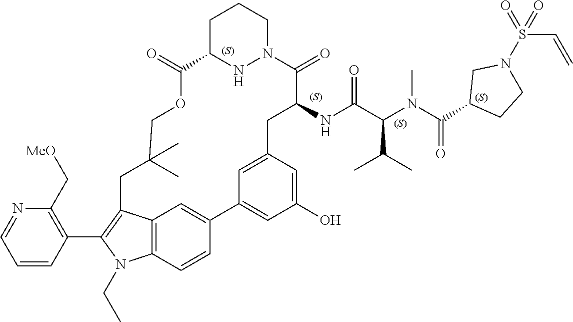 |
| BA70 | 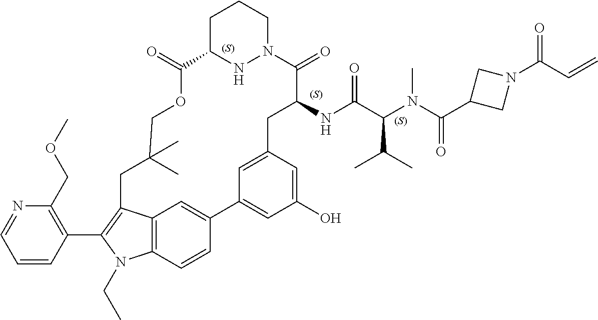 |
| BA71 | 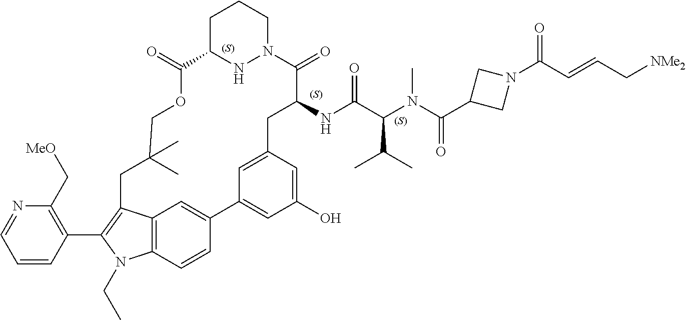 |
| BA72 | 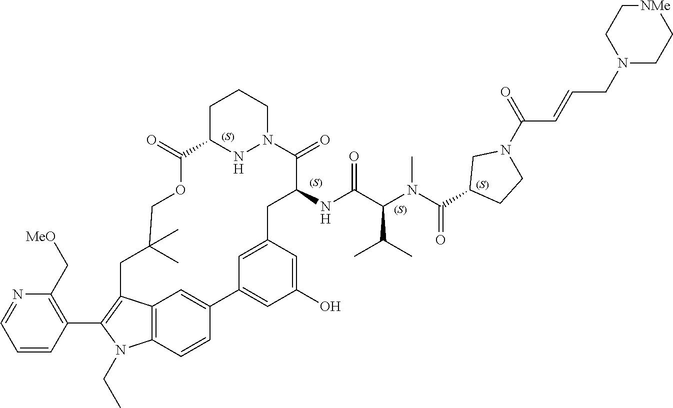 |
| BA73 | 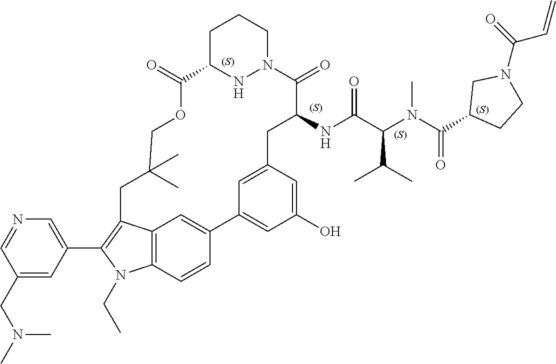 |
| BA74 | 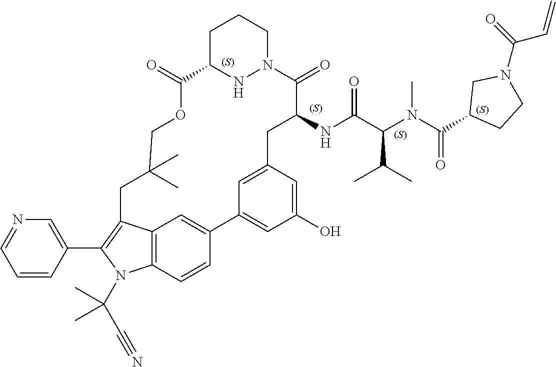 |
| BA75 | 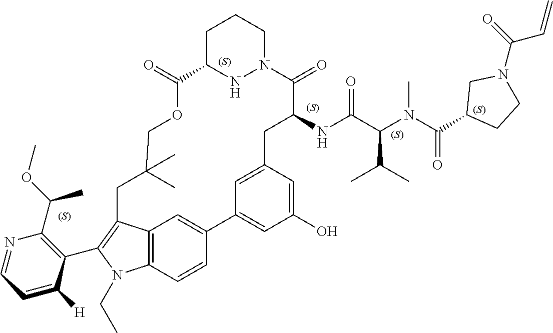 |
| BA76 | 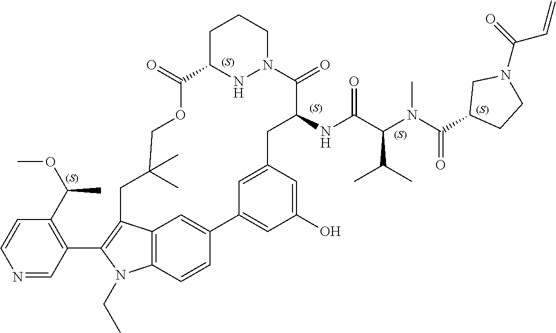 |
| BA77 | 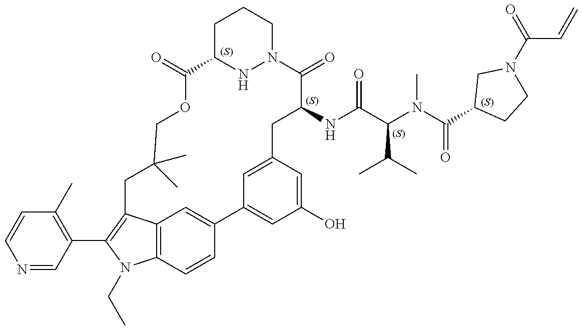 |
| BA78 | 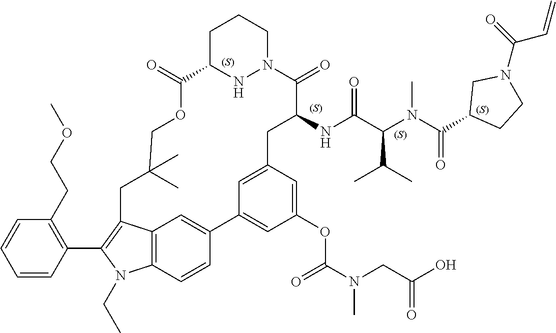 |
| BA79 | 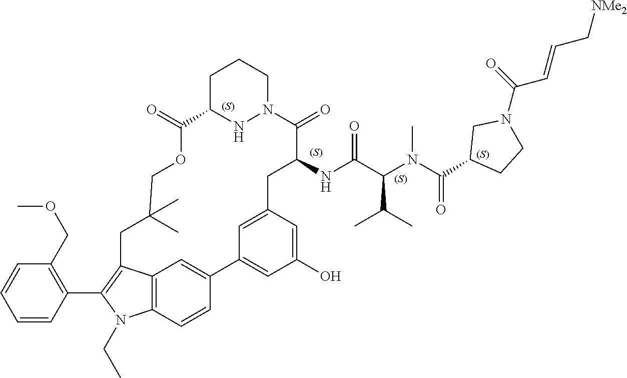 |
| BA80 | 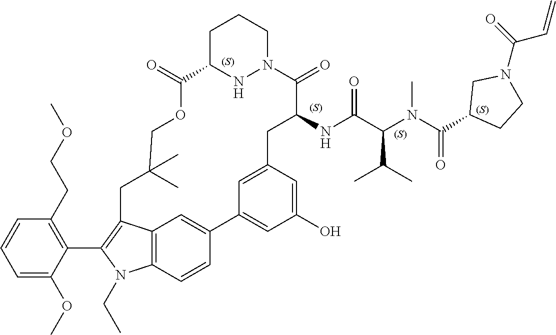 |
| BA81 | 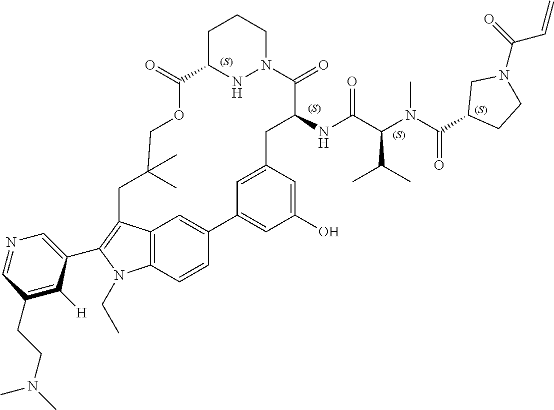 |
| BA82 | 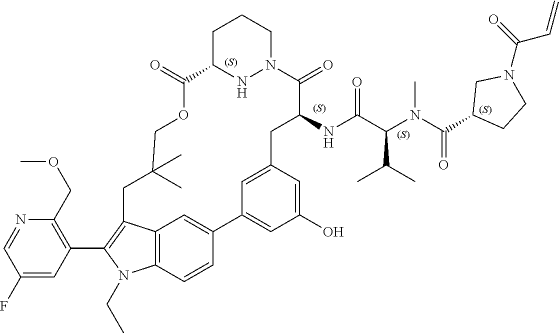 |
| BA83 | 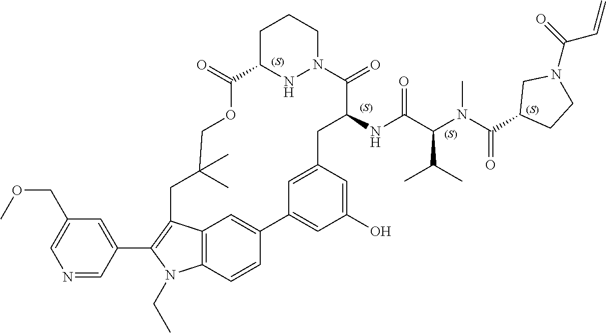 |
| BA84 | 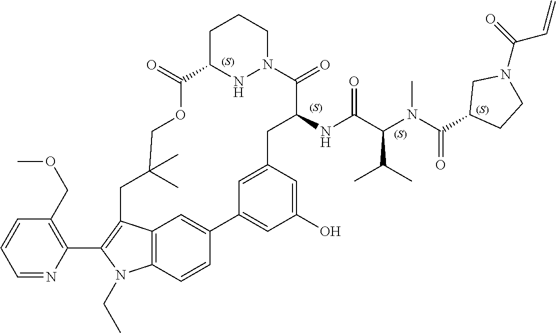 |
| BA85 | 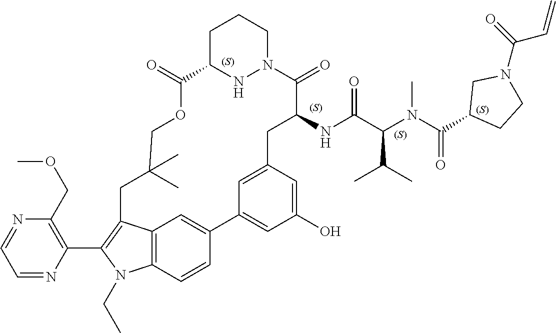 |
| BA86 | 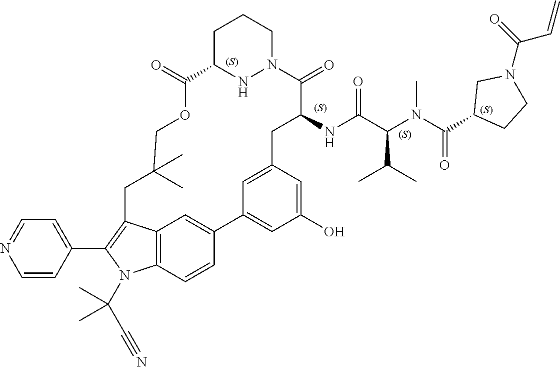 |
| BA87 | 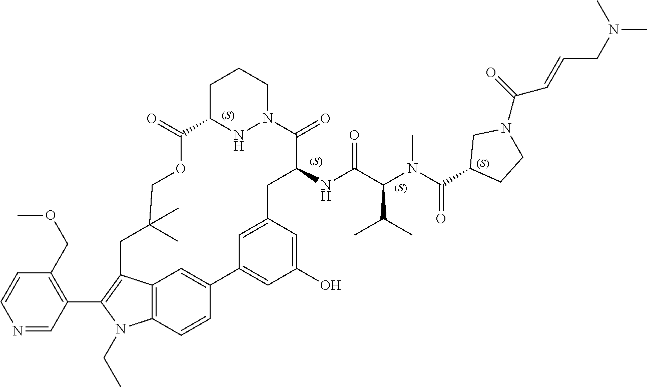 |
| BA88 | 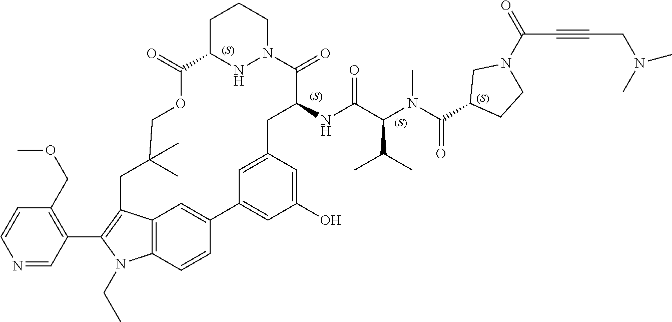 |
| BA89 | 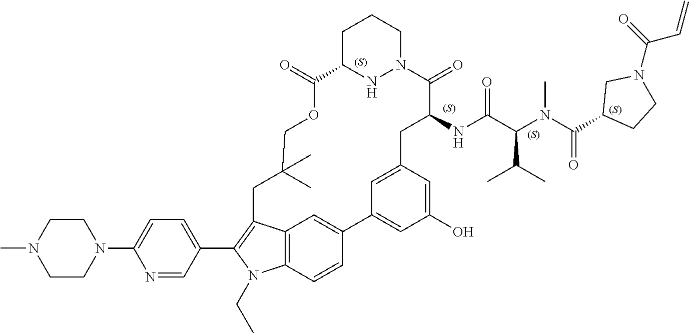 |
| BA90 | 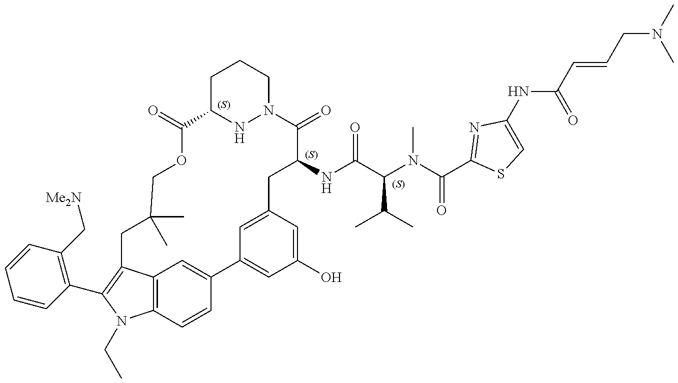 |
| BA91 | 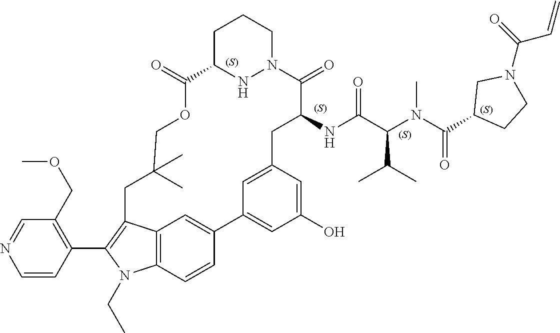 |
| BA92 | 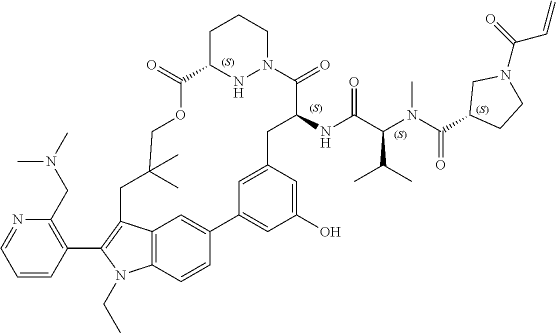 |
| BA93 | 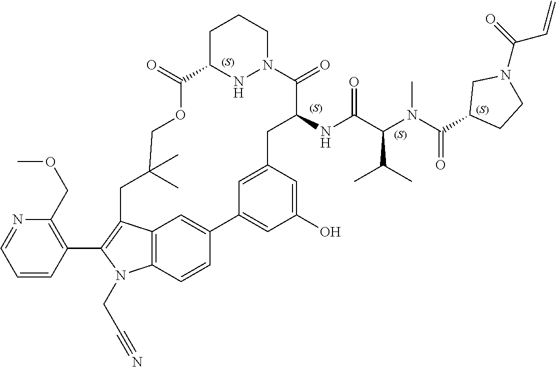 |
| BA94 | 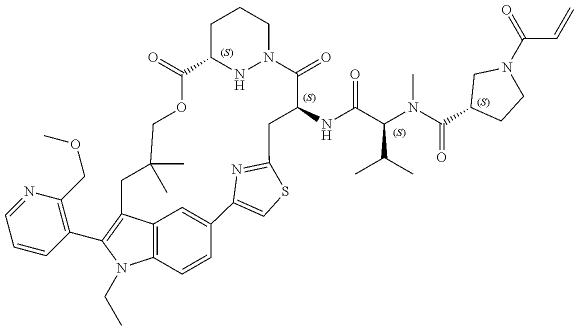 |
| BA95 | 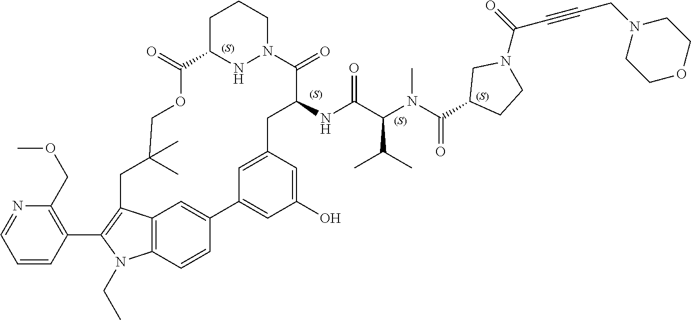 |
| BA96 | 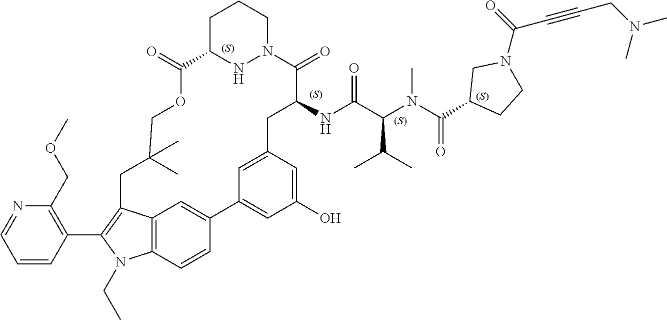 |
| BA97 | 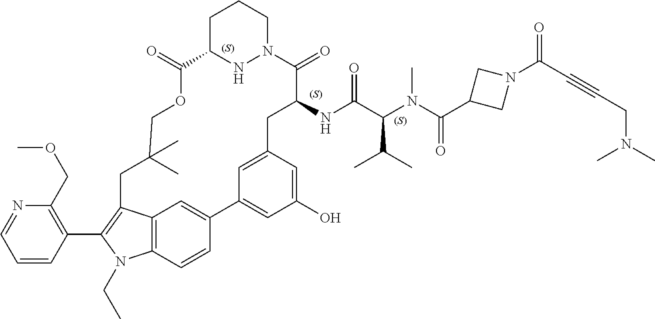 |
| BA98 | 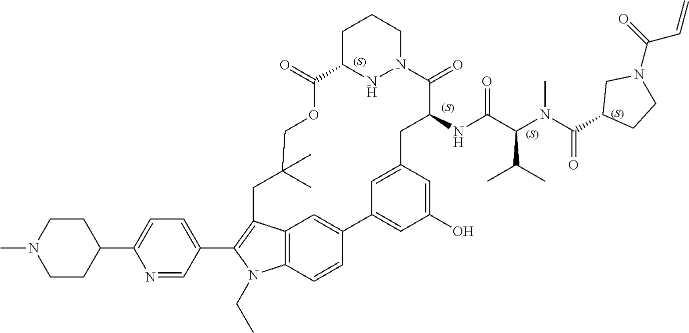 |
| BA99 | 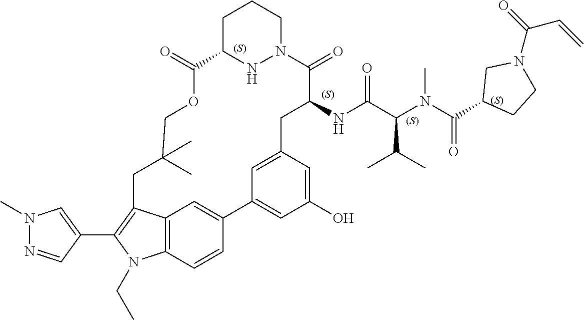 |
| BA100 | 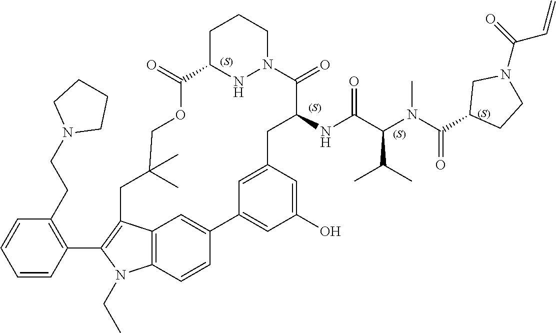 |
| BA101 | 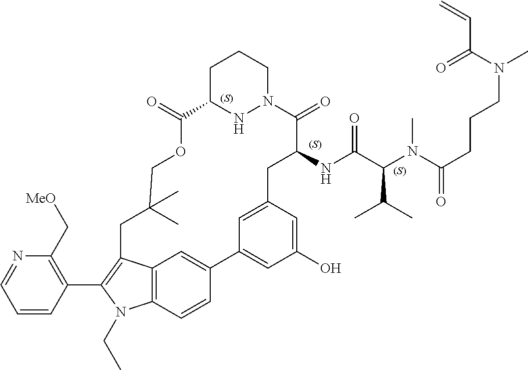 |
| BA102 | 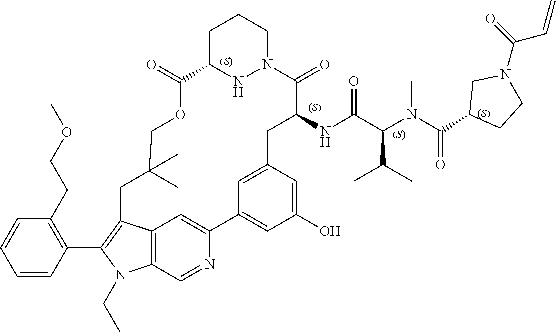 |
| BA103 | 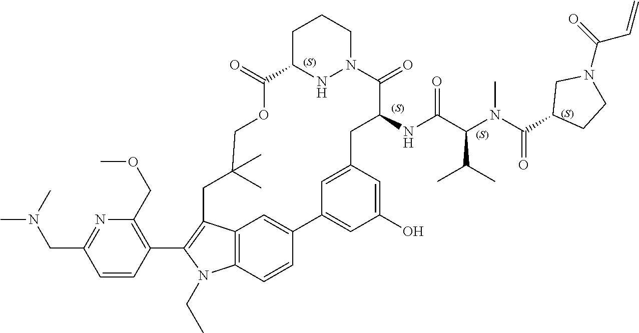 |
| BA104 | 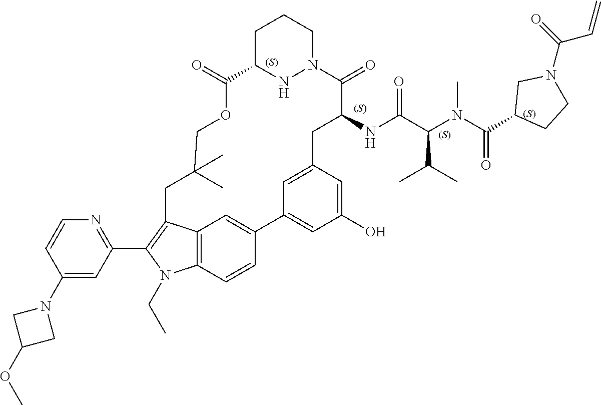 |
| BA105 | 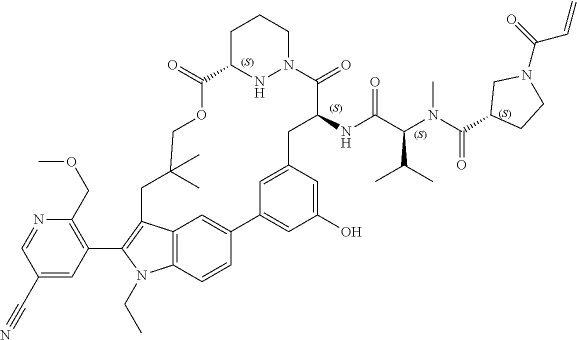 |
| BA106 | 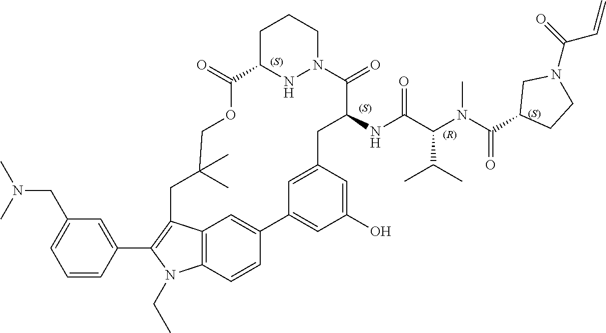 |
| BA107 | 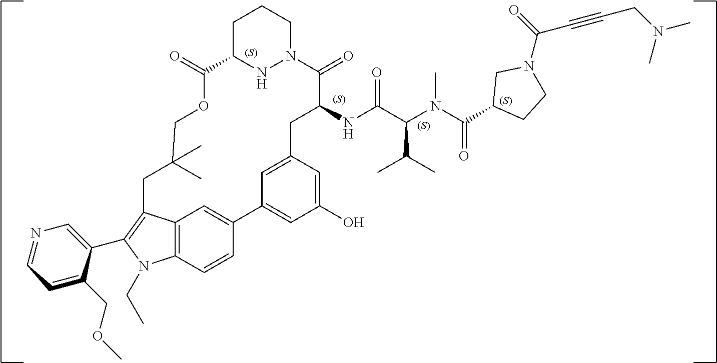 |
| BA108 | 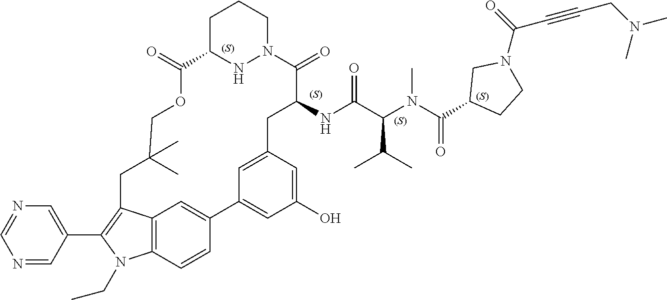 |
| BA109 | 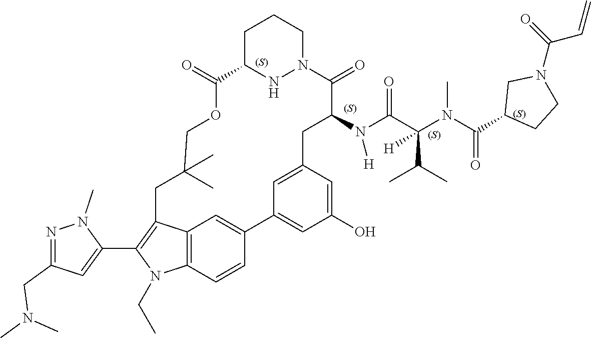 |
| BA110 | 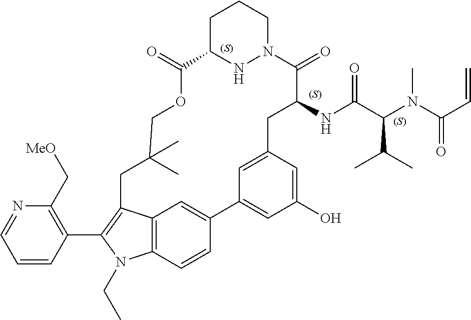 |
| BA111 | 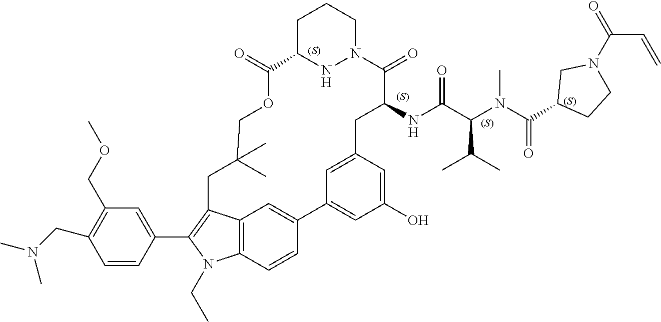 |
| BA112 | 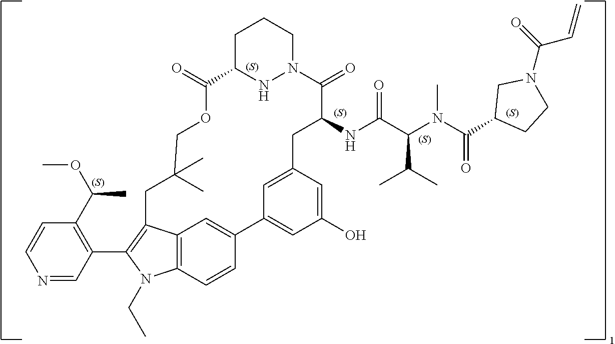 |
| BA113 | 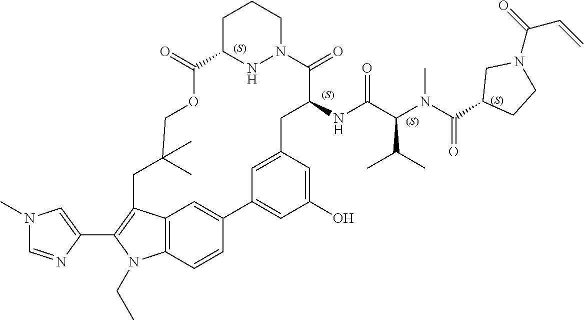 |
| BA114 | 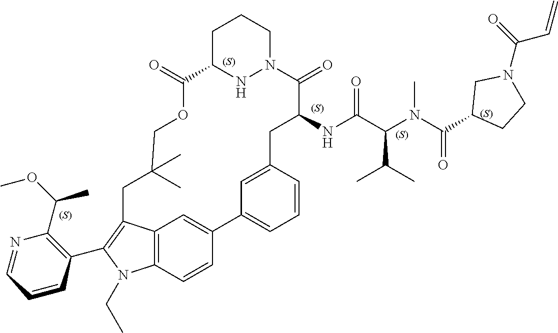 |
| BA115 | 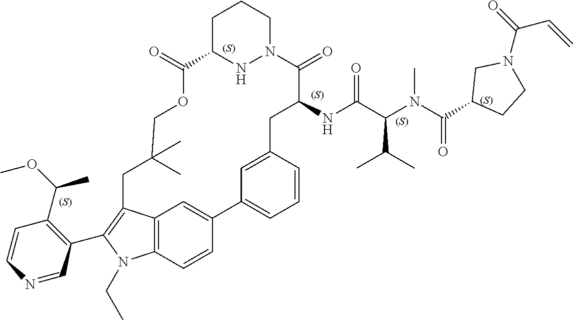 |
| BA116 | 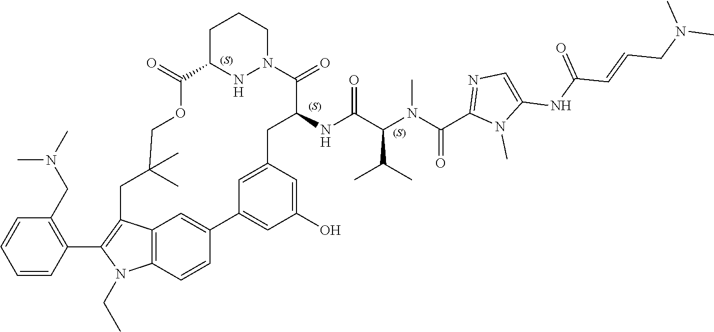 |
| BA117 | 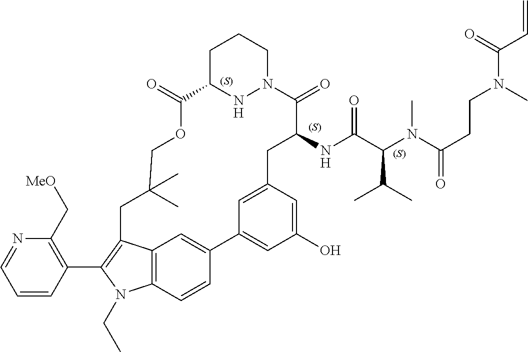 |
| BA118 | 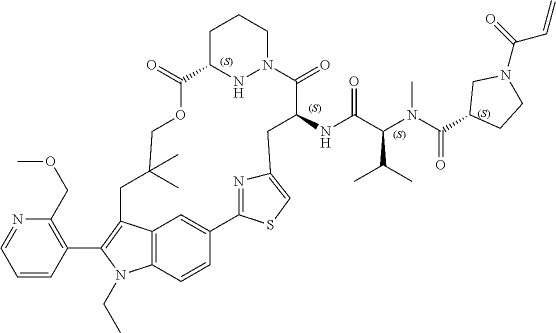 |
| BA119 | 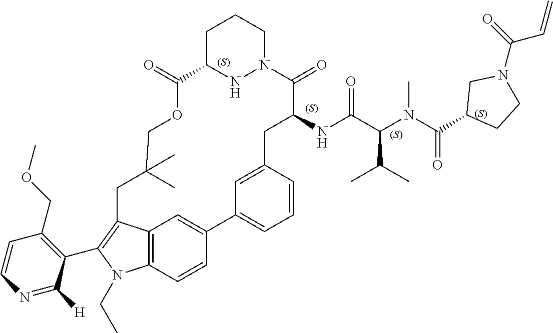 |
| BA120 | 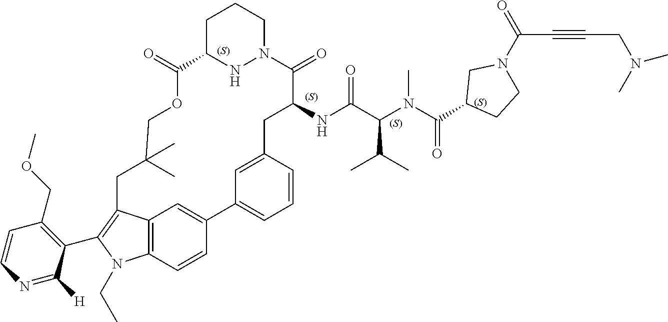 |
| BA121 | 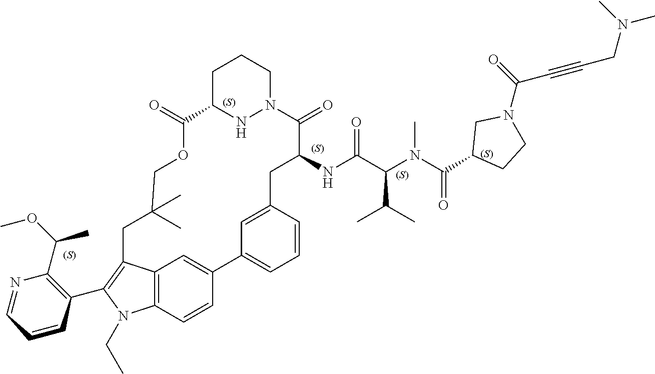 |
| BA122 | 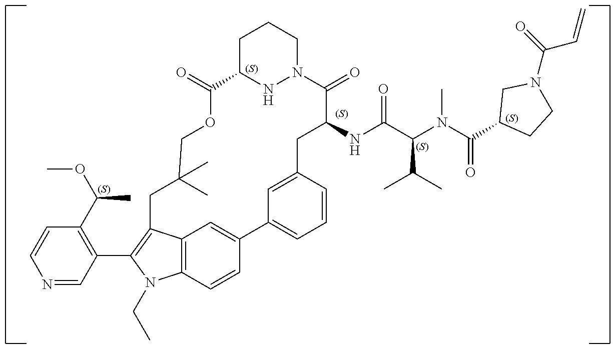 |
| BA123 | 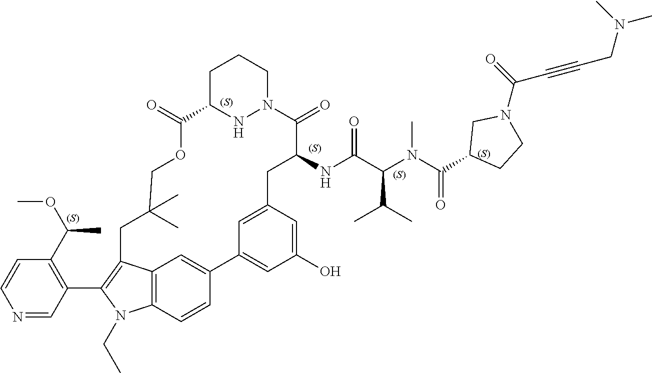 |
| BA124 | 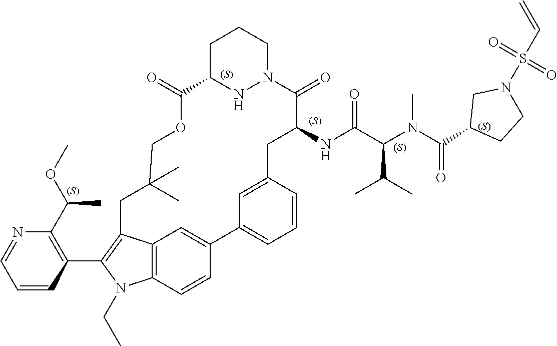 |
| BA125 | 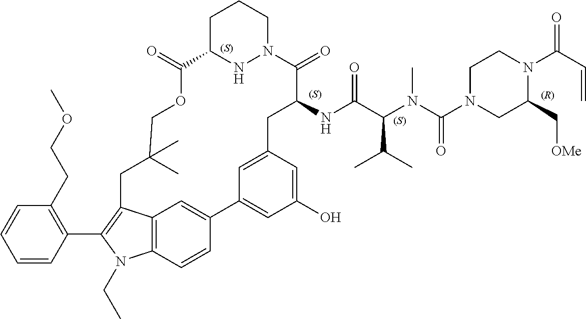 |
| BA126 | 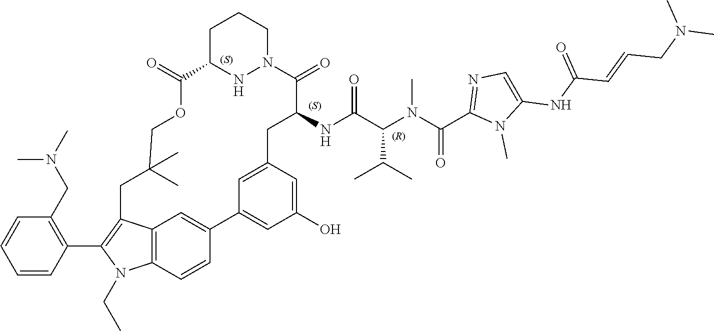 |
| BA127 | 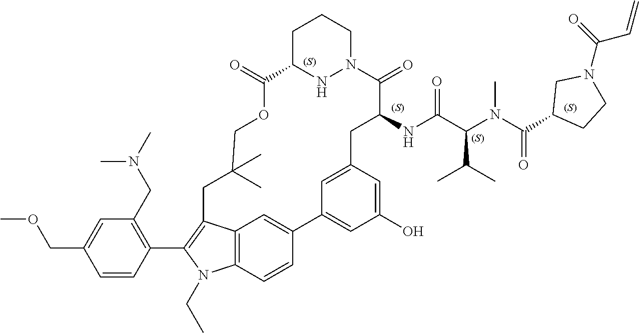 |
| BA128 | 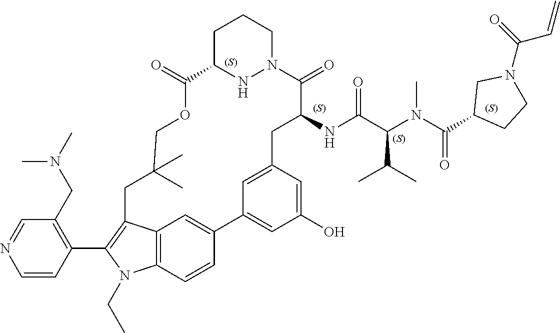 |
| BA129 | 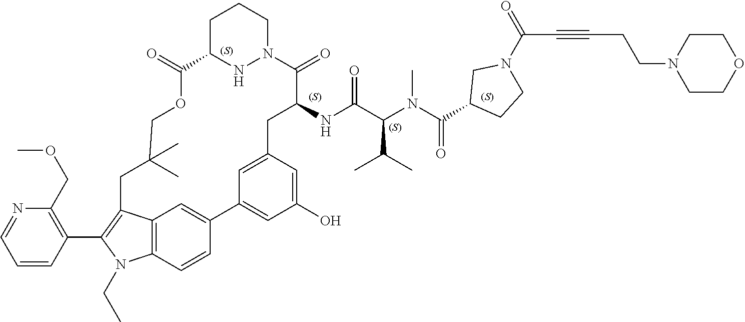 |
| BA130 | 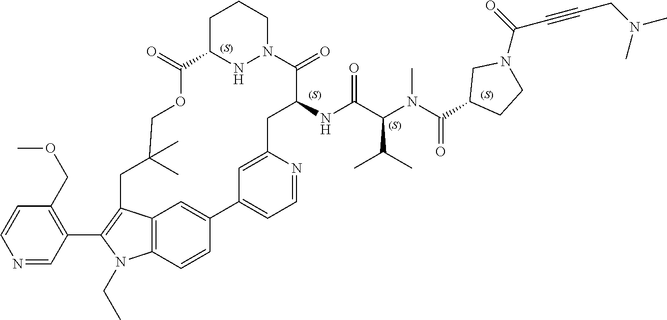 |
| BA131 | 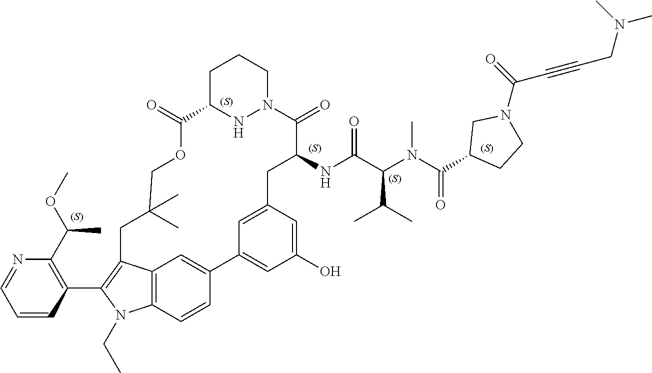 |
| BA132 | 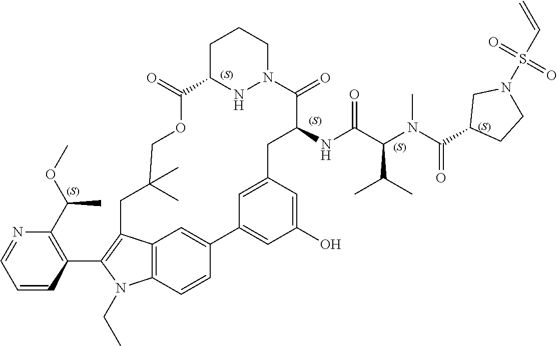 |
| BA133 | 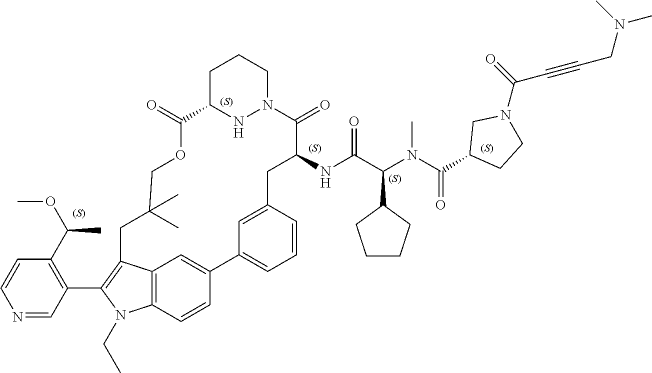 |
| BA134 | 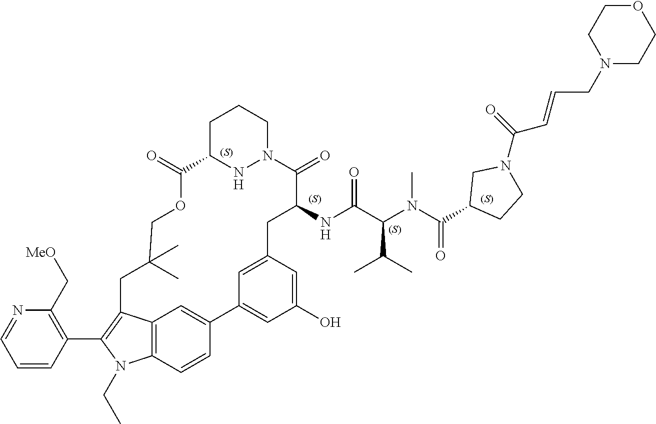 |
| BA135 | 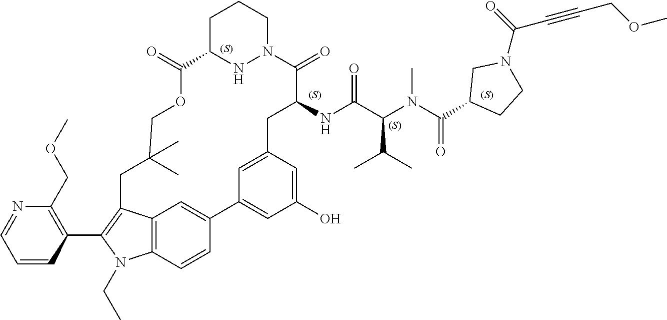 |
| BA136 | 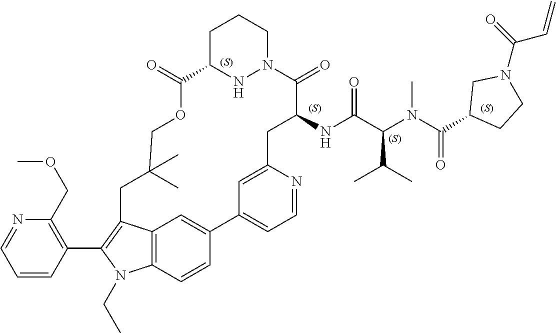 |
| BA137 | 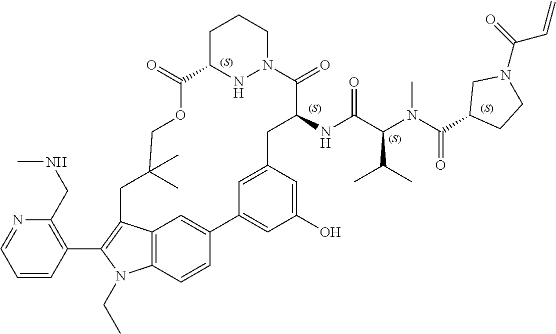 |
| BA138 | 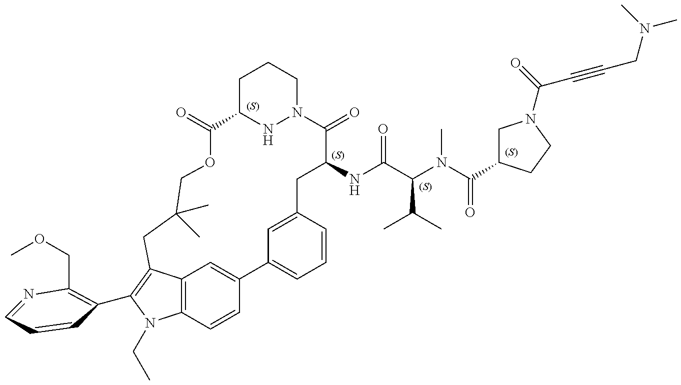 |
| BA139 | 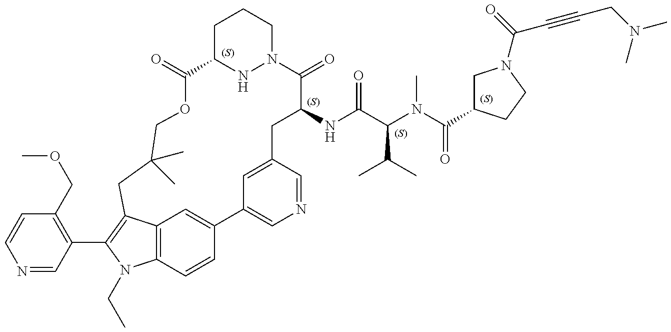 |
| BA140 | 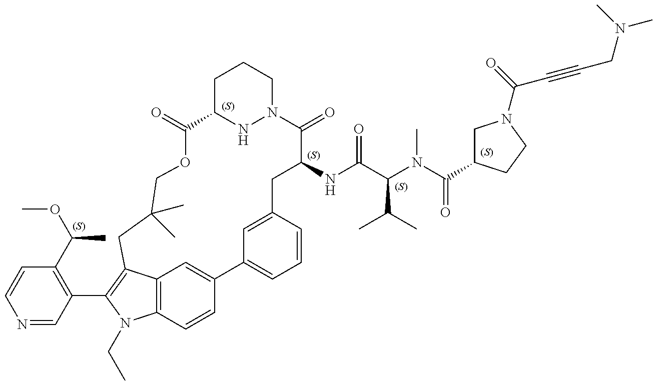 |
| BA141 | 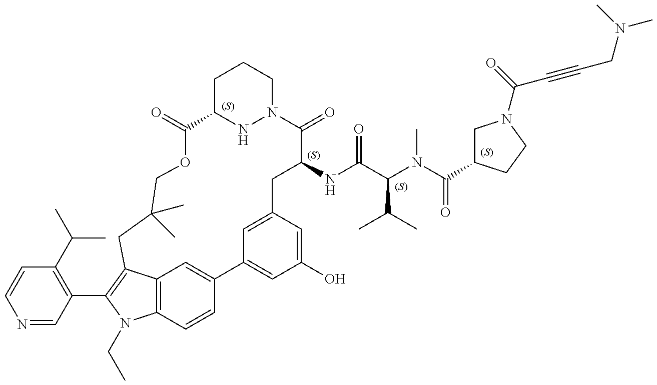 |
| BA142 | 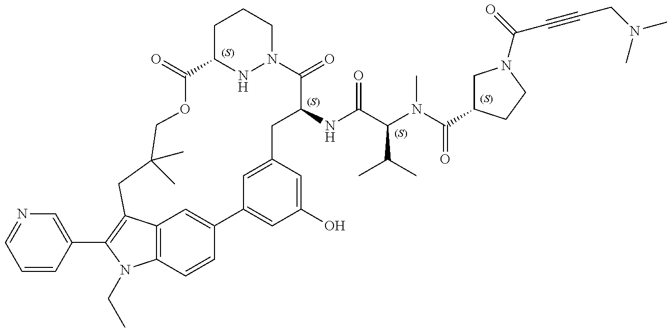 |
| BA143 | 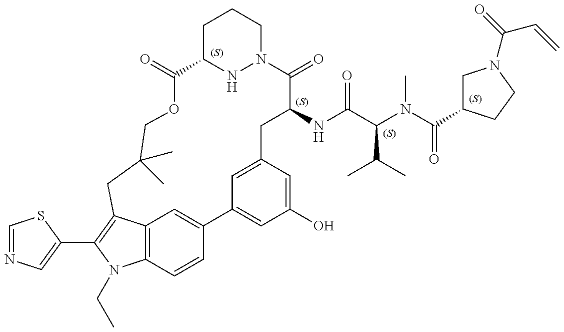 |
| BA144 | 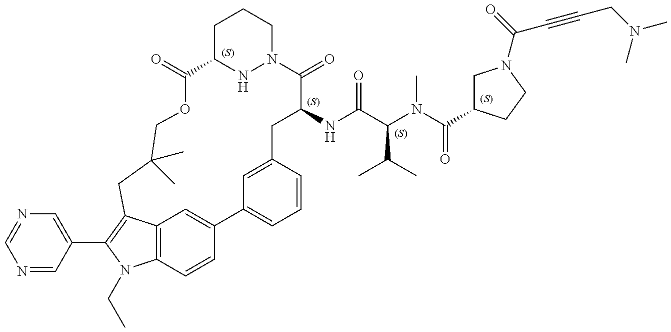 |
| BA145 | 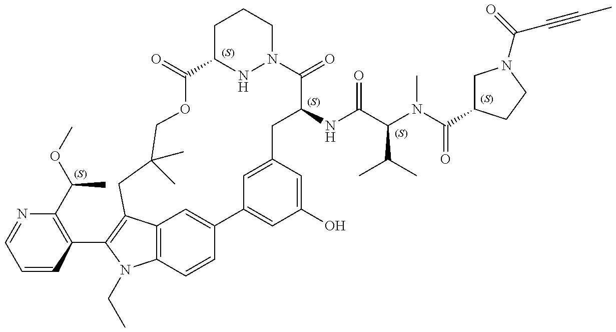 |
| BA146 | 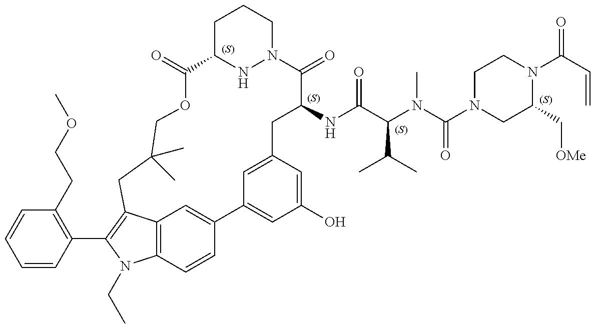 |
| BA147 | 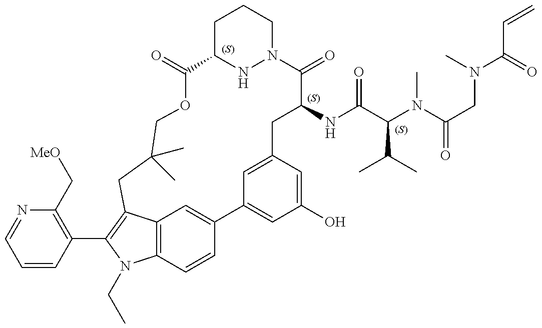 |
| BA148 | 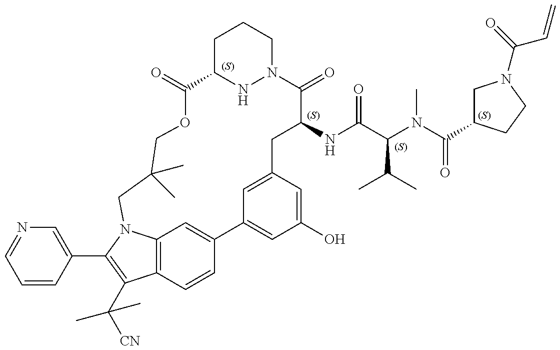 |
| BA149 | 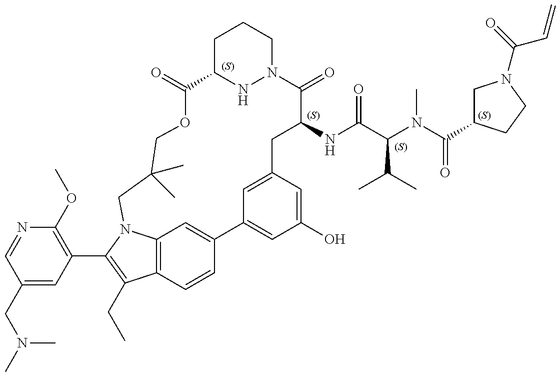 |
| BA150 | 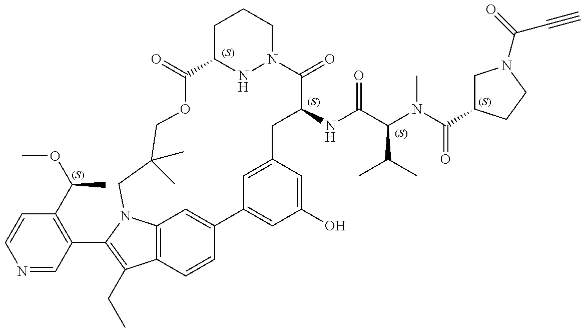 |
| BA151 | 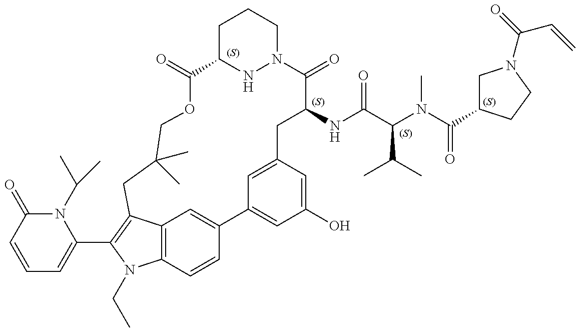 |
| BA152 | 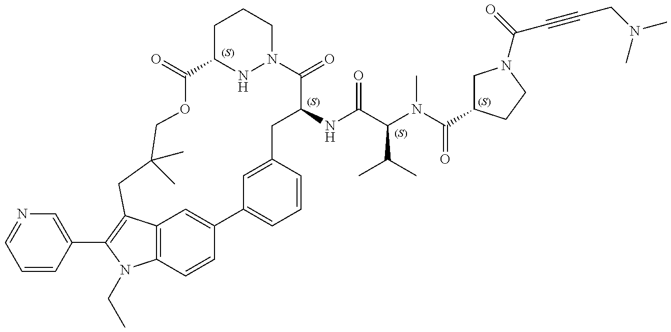 |
| BA153 | 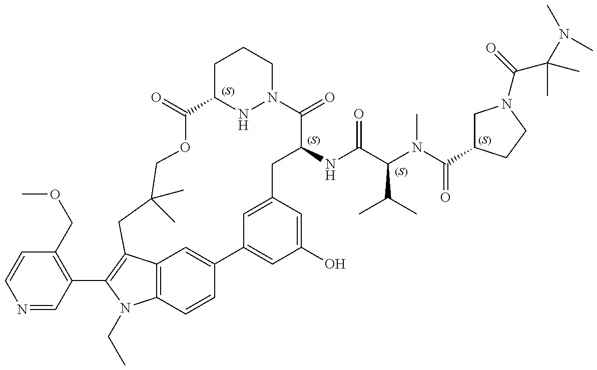 |
| BA154 | 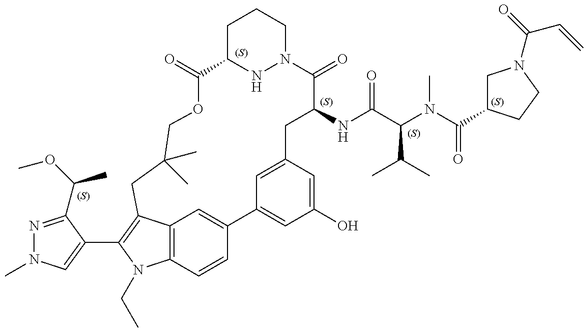 |
| BA155 | 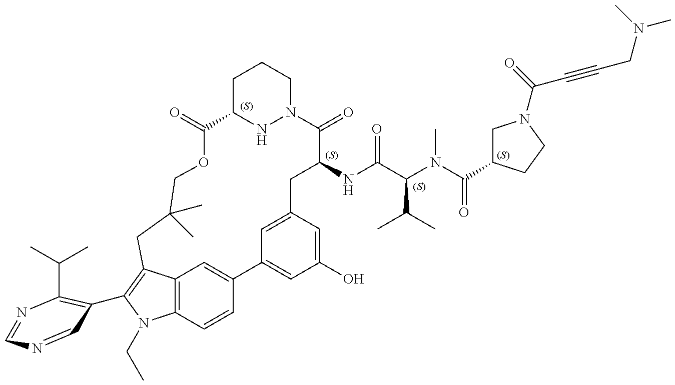 |
| BA156 | 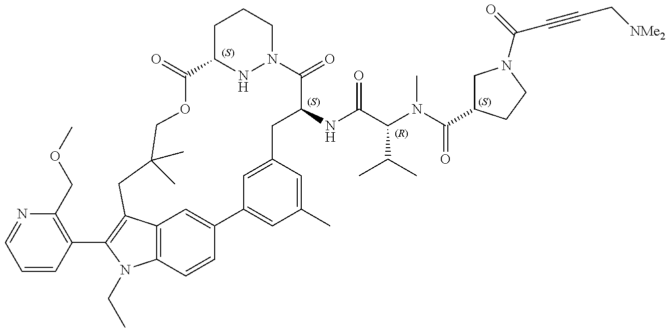 |
| BA157 | 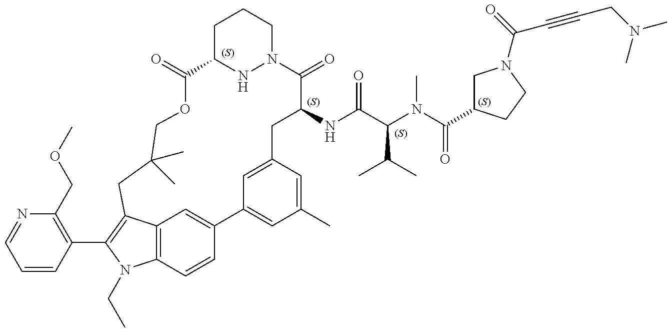 |
| BA158 | 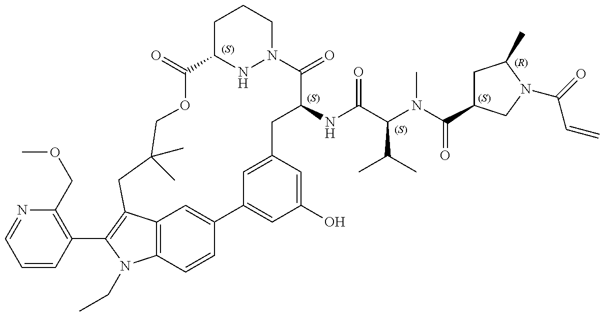 |
| BA159 | 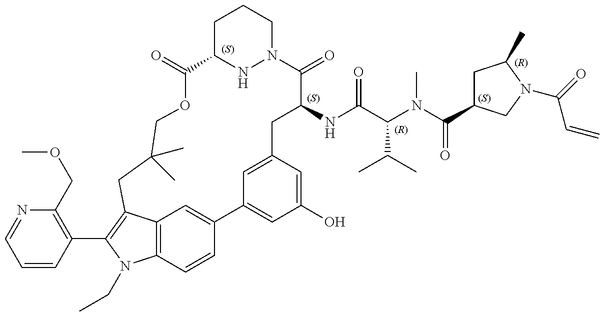 |
| BA160 | 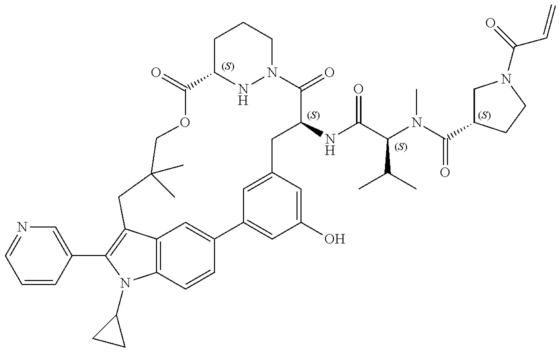 |
| BA161 | 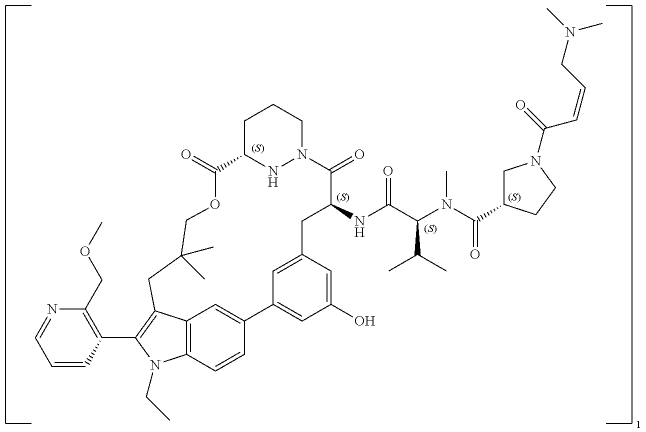 |
| BA162 | 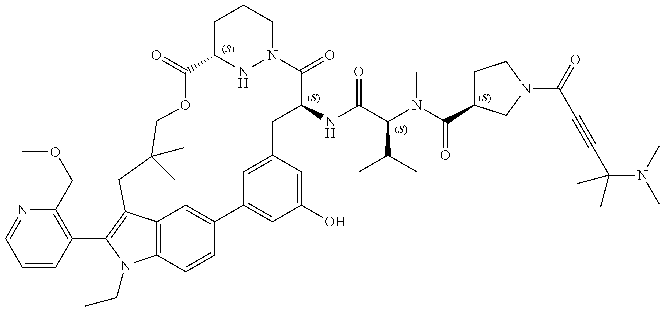 |
| BA163 | 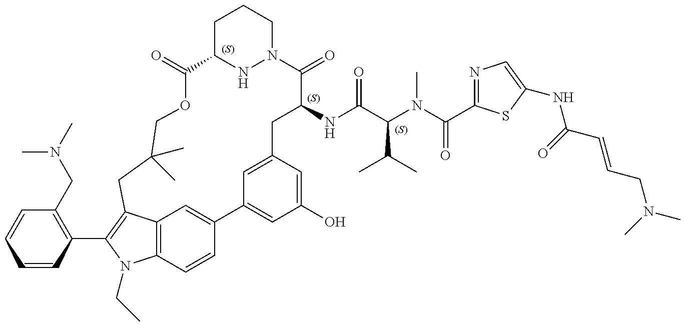 |
| BA164 | 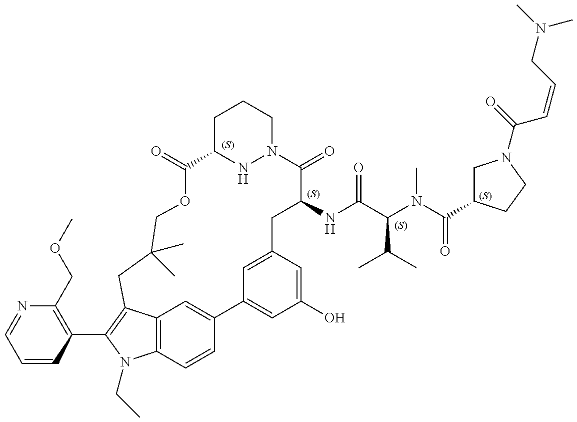 |
| BA165 | 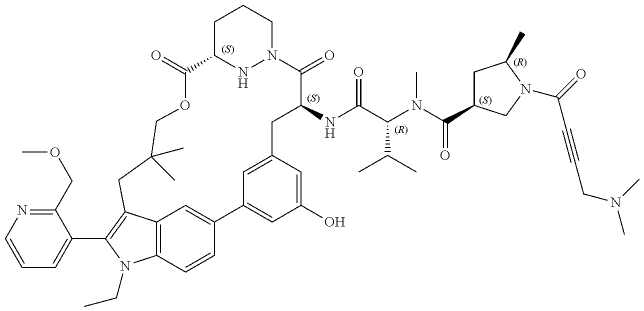 |
| BA166 | 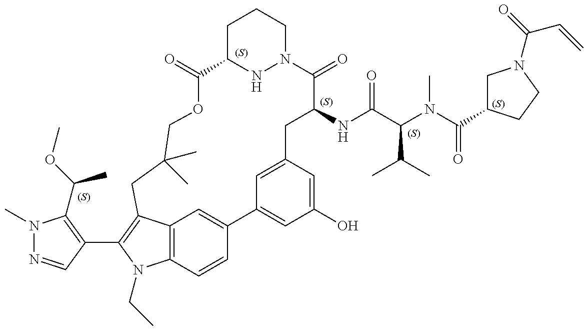 |
| BA167 | 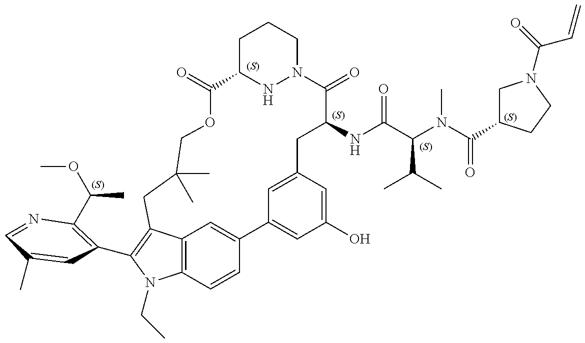 |
| BA168 | 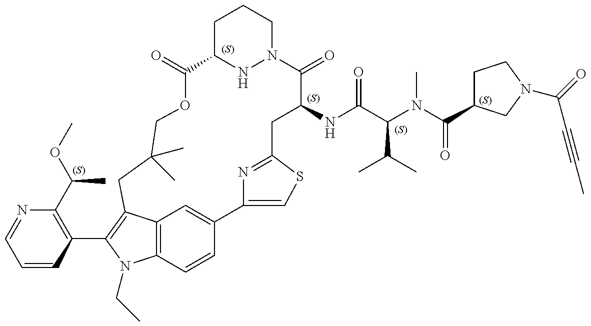 |
| BA169 | 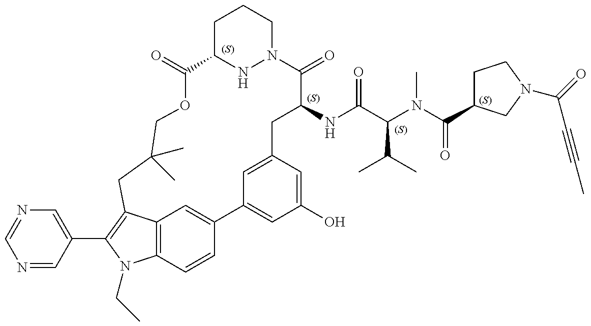 |
| BA170 | 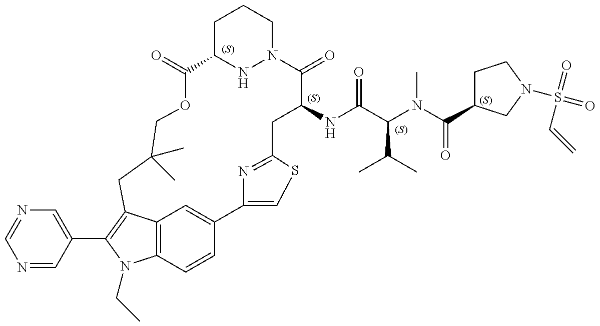 |
| BA171 | 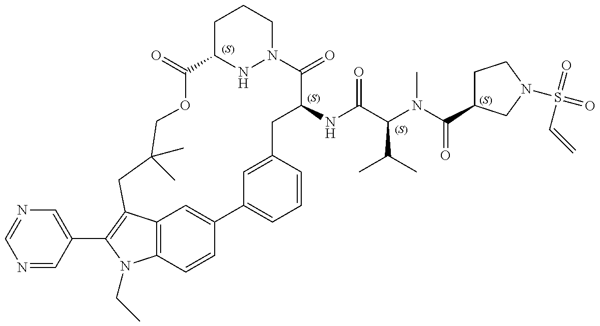 |
| BA172 | 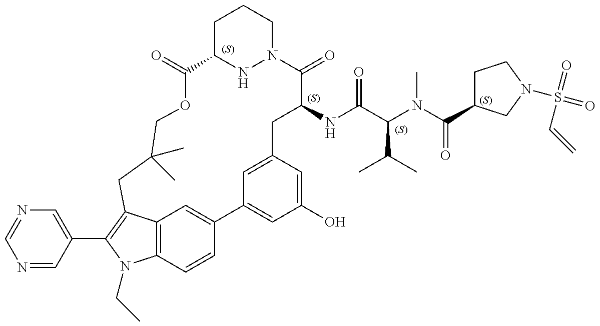 |
| BA173 | 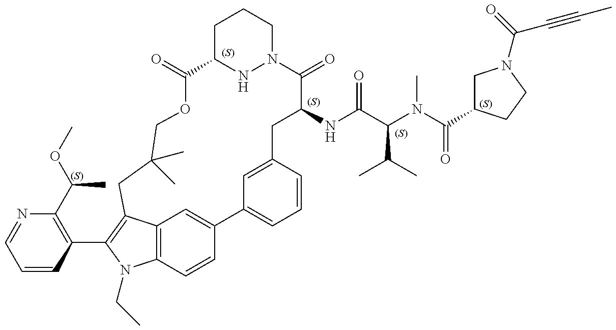 |
| BA174 | 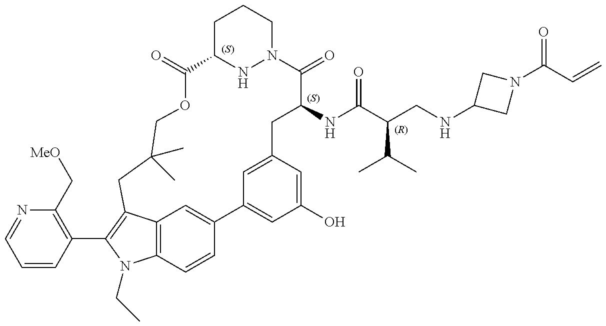 |
| BA175 | 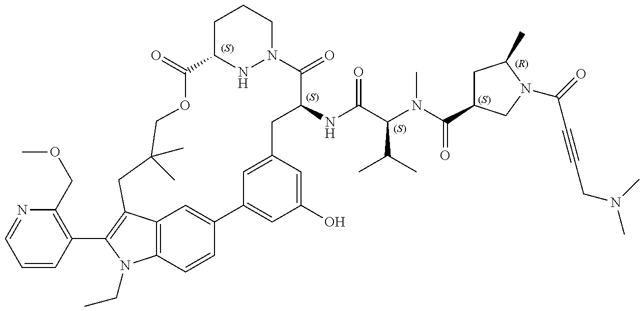 |
| BA176 | 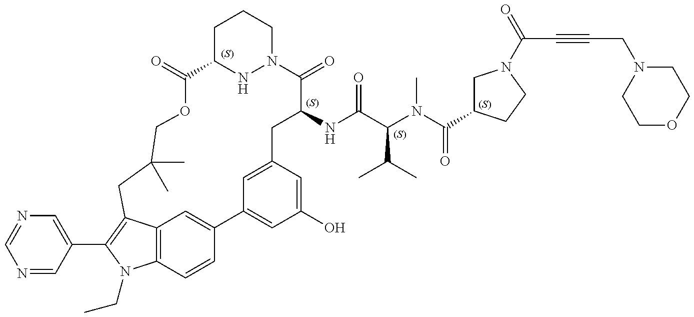 |
| BA177 | 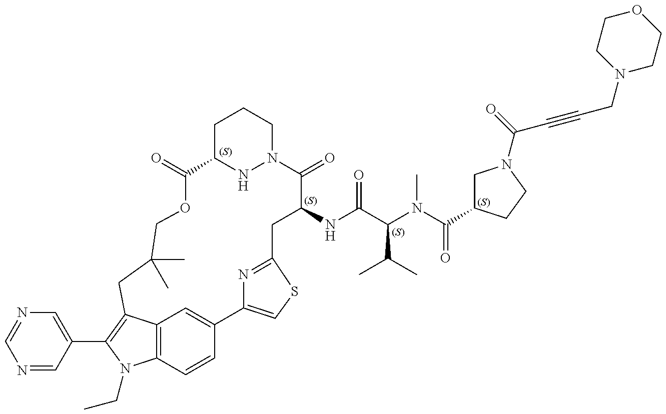 |
| BA178 | 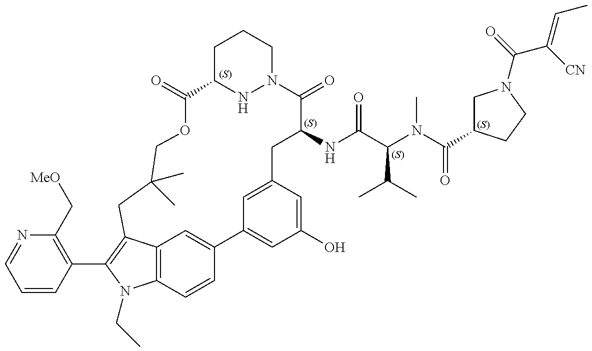 |
| BA179 | 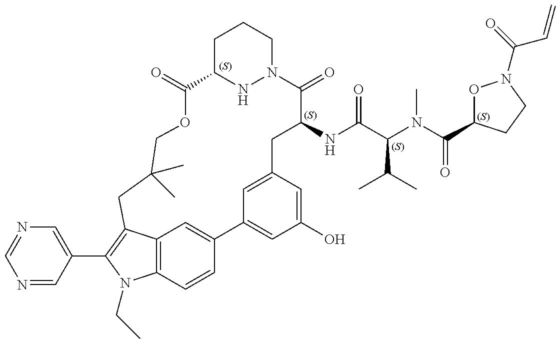 |
| BA180 | 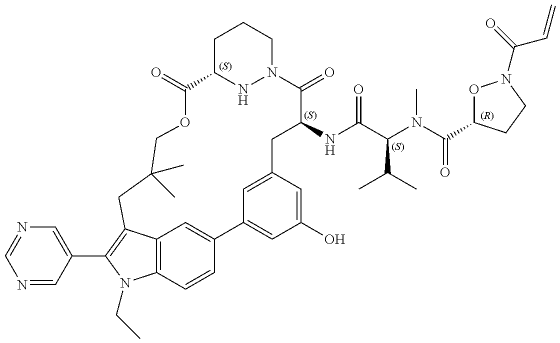 |
| BA181 | 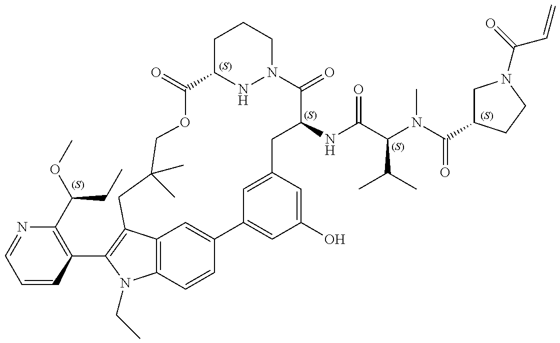 |
| BA182 | 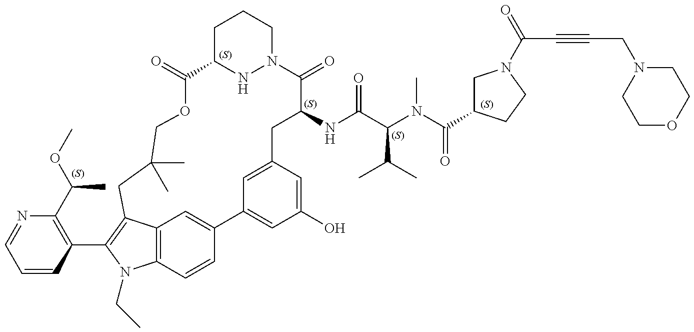 |
| BA183 | 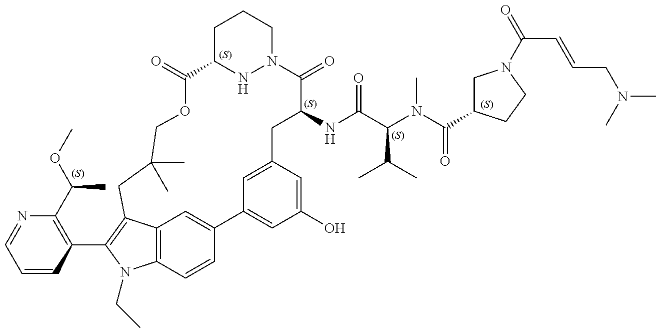 |
| BA184 | 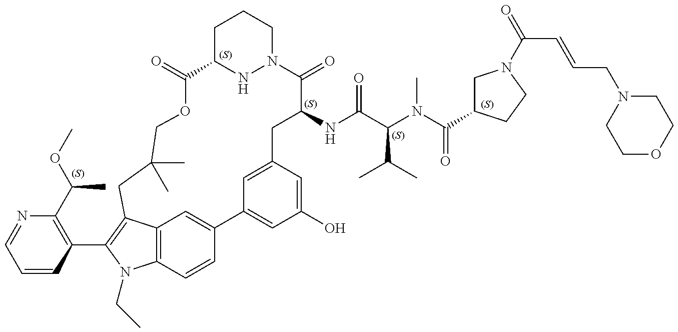 |
| BA185 | 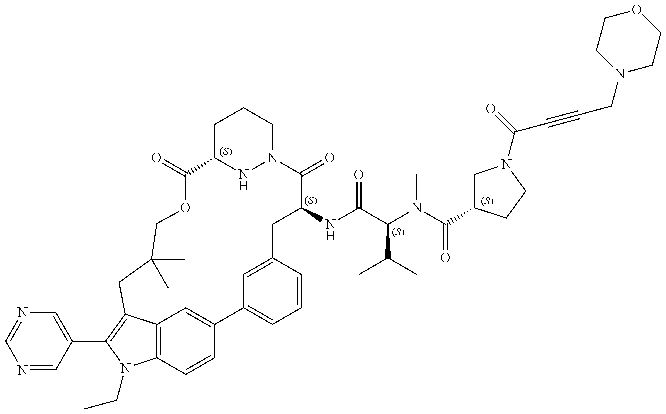 |
| BA186 | 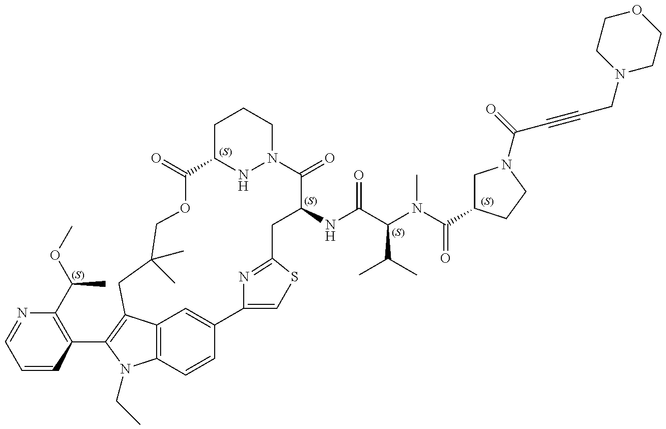 |
| BA187 | 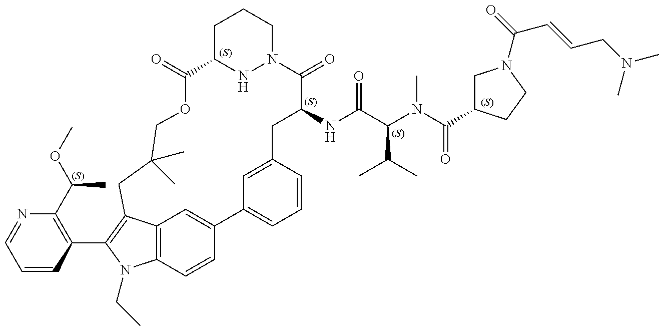 |
| BA188 | 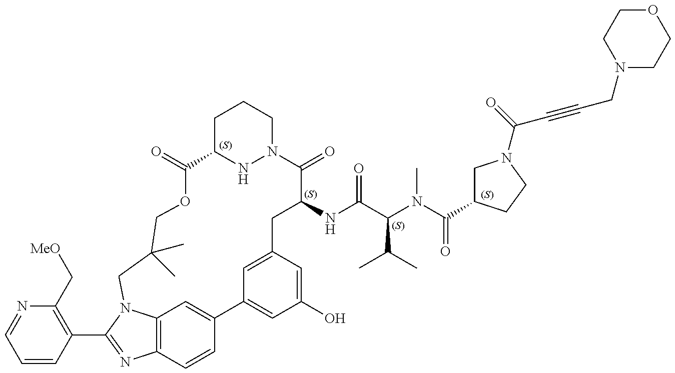 |
| BA189 | 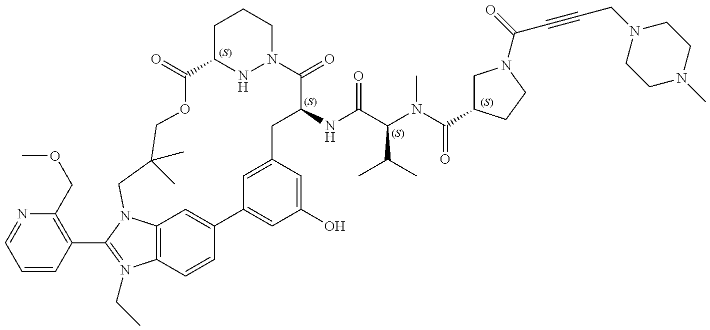 |
| BA190 | 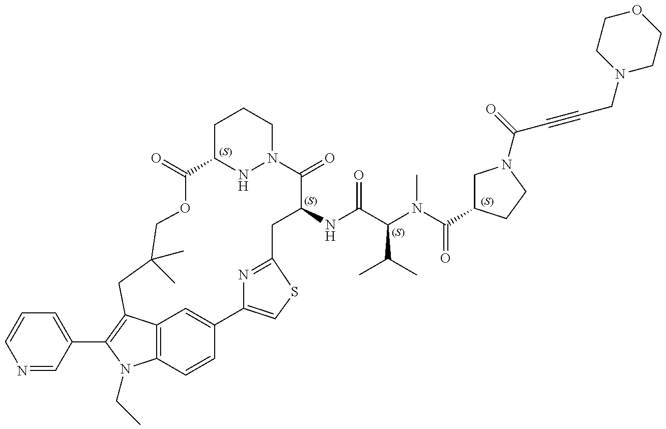 |
| BA191 | 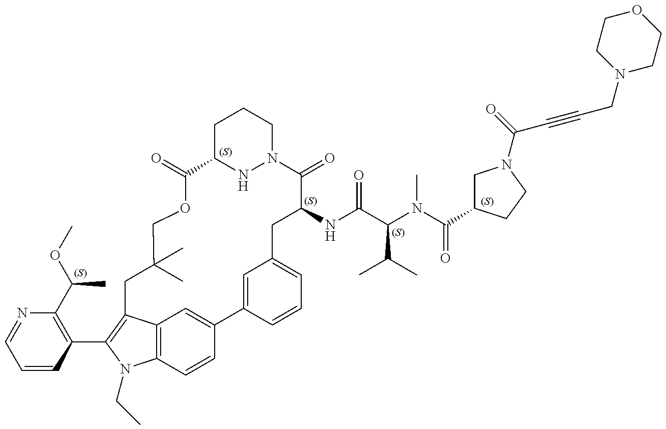 |
| BA192 | 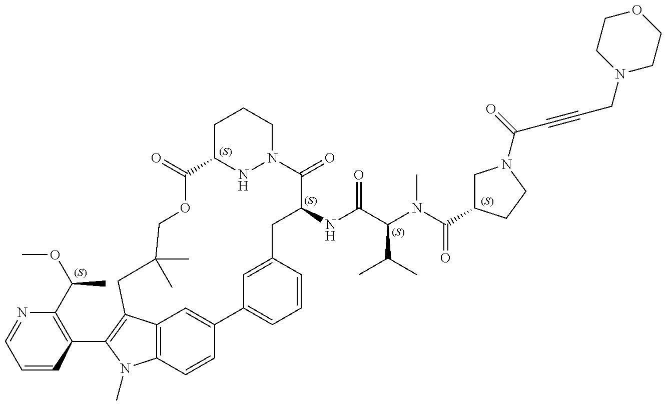 |
| BA193 | 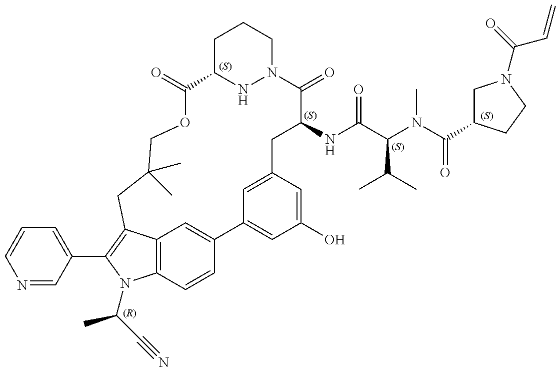 |
| BA194 | 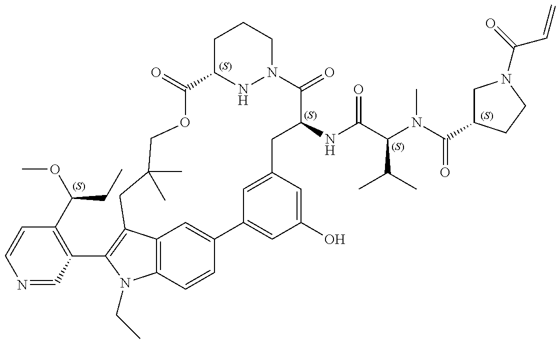 |
| BA195 | 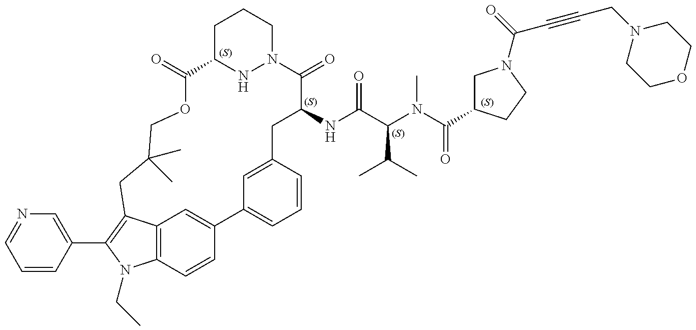 |
| BA196 | 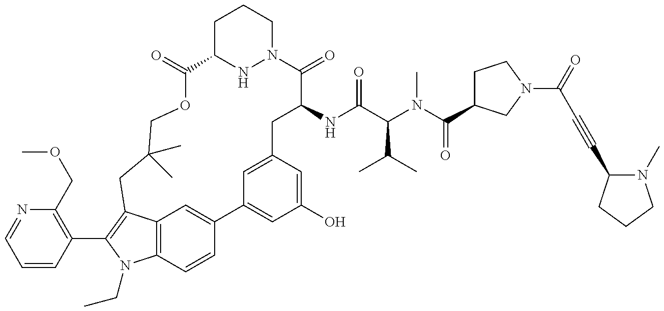 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BA197 | 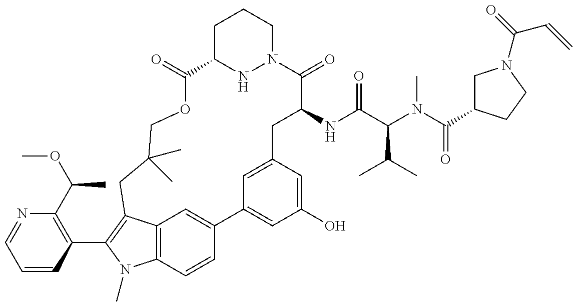 |
| BA198 | 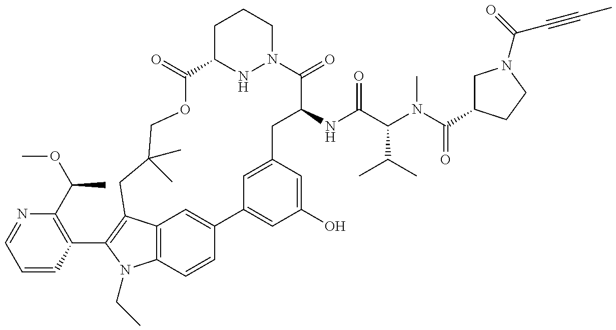 |
| BA199 | 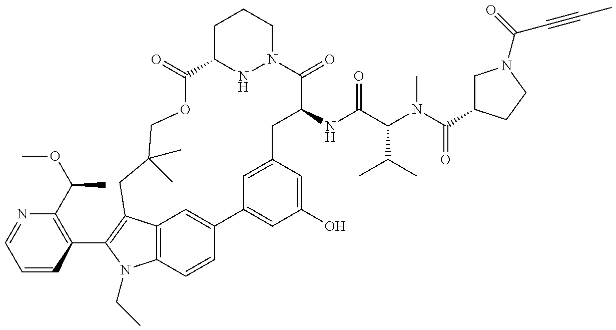 |
| BA200 | 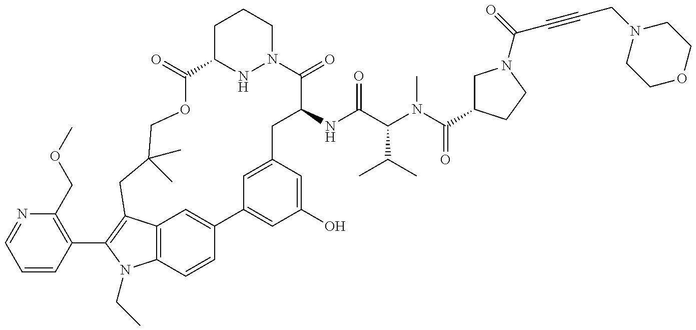 [SMILES: CCn1c(-c2cccnc2COC)c2c3cc(ccc31)-c1cc(O)cc(c1)C[C@H](NC(=O)[C@@H](C(C)C)N(C)C(=O)[C@H]1CCN(C(=O)C#CCN3CCOCC3)C1)C(=O)N1CCC[C@H](N1)C(=O)OCC(C)(C)C2] |
| BA201 | 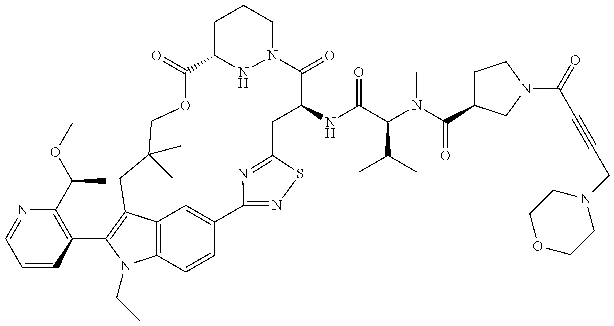 |
| BA202 | 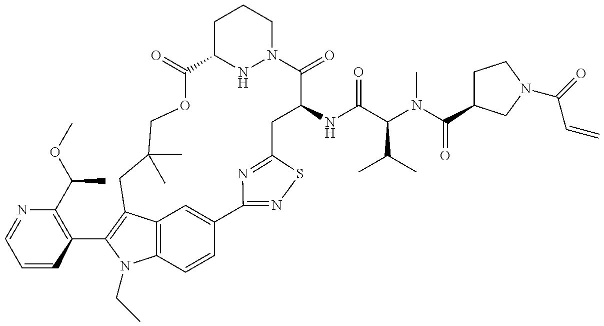 |
| BA203 | 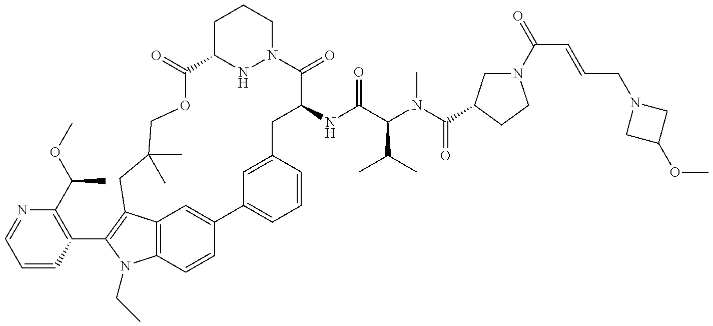 |
| BA204 | 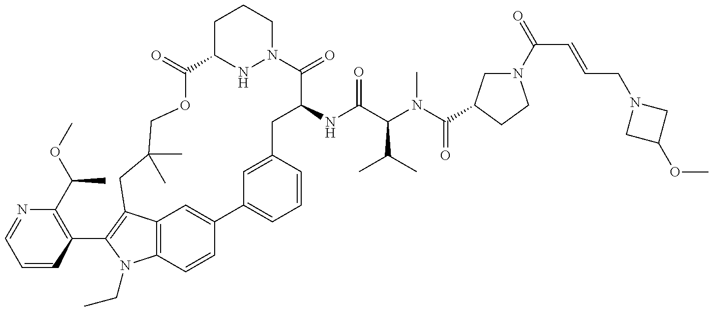 |
| BA205 | 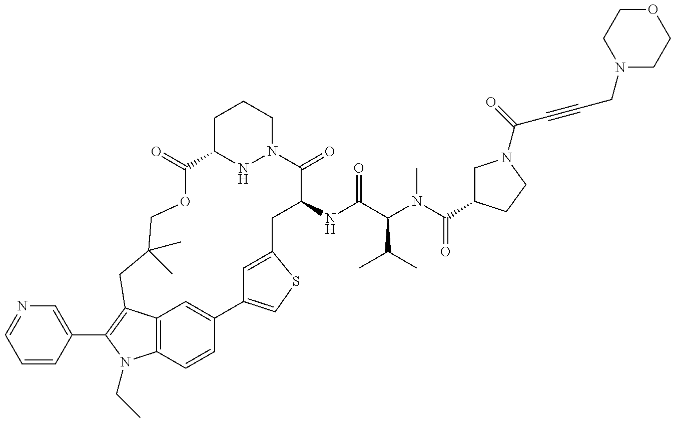 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BA206 | 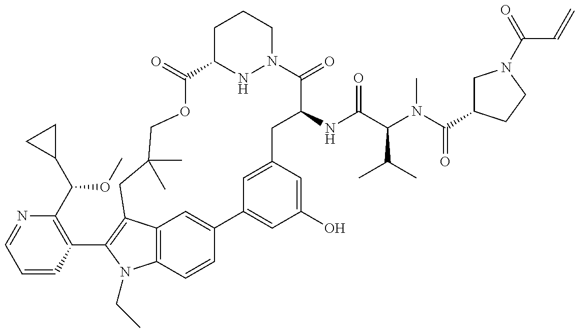 |
| BA207 | 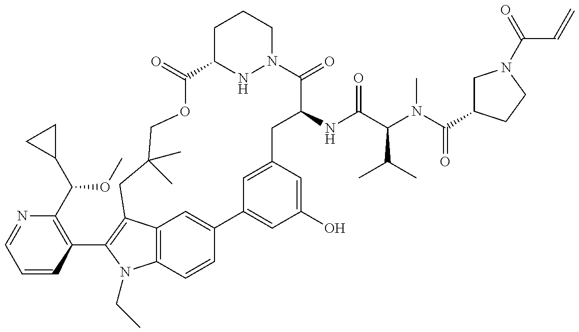 |
| BA208 | 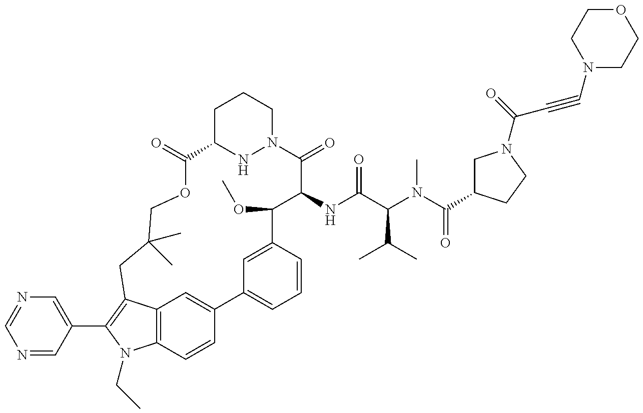 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CN3CCOCC3)C1)C(=O)N1CCC[C@H](N1)C(=O)OCC(C)(C)C2] |
| BA209 | 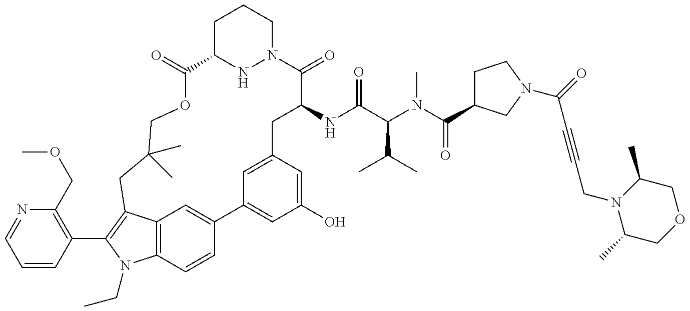 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@@H](C)COC[C@@H]3C)C1)C(=O)N1CCC[C@H](N1)C(=O)OCC(C)(C)C2] |
| BA210 | 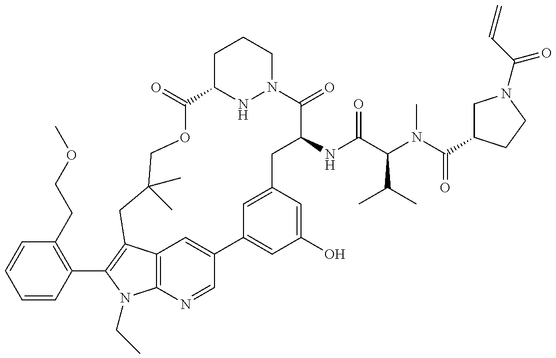 [SMILES: C=CC(=O)N1CC[C@H](C(=O)N(C)[C@H](C(=O)N[C@H]2Cc3cc(O)cc(c3)-c3cnc4c(c3)c(c(-c3ccccc3CCOC)n4CC)CC(C)(C)COC(=O)[C@@H]3CCCN(N3)C2=O)C(C)C)C1] |
| BA211 | 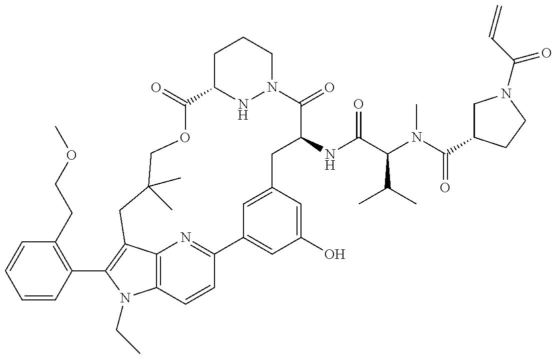 [SMILES: C=CC(=O)N1CC[C@H](C(=O)N(C)[C@H](C(=O)N[C@H]2Cc3cc(O)cc(c3)-c3ccc4c(n3)c(c(-c3ccccc3CCOC)n4CC)CC(C)(C)COC(=O)[C@@H]3CCCN(N3)C2=O)C(C)C)C1] |
| BA212 | 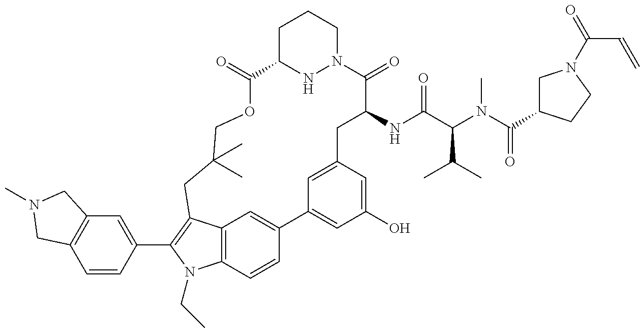 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)CN(C)C5)n4CC)CC(C)(C)COC(=O)[C@@H]3CCCN(N3)C2=O)C(C)C)C1] |
| BA213 | 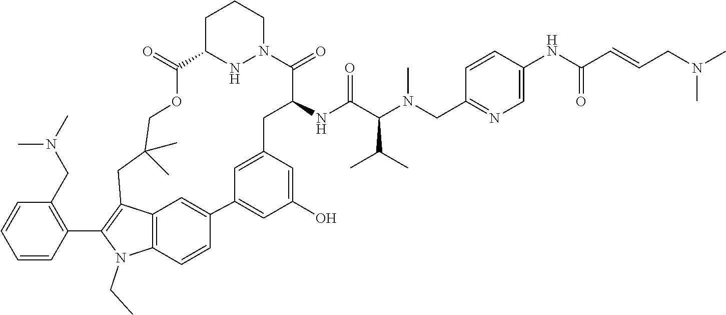 [SMILES: CCn1c(-c2ccccc2CN(C)C)c2c3cc(ccc31)-c1cc(O)cc(c1)C[C@H](NC(=O)[C@H](C(C)C)N(C)Cc1ccc(NC(=O)/C=C/CN(C)C)cn1)C(=O)N1CCC[C@H](N1)C(=O)OCC(C)(C)C2] |
| BA214 | 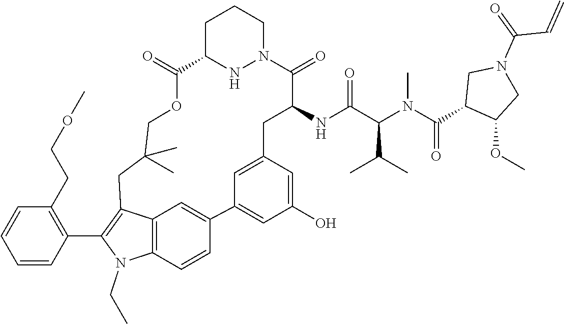 [SMILES: C=CC(=O)N1C[C@H](OC)[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA215 | 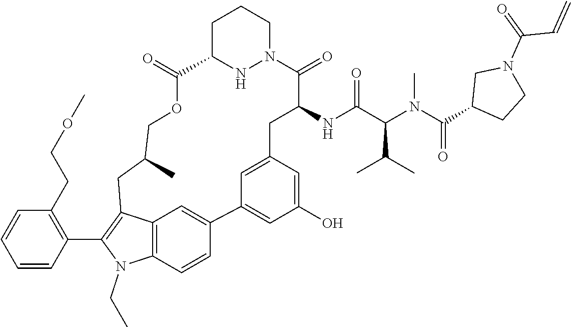 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)C[C@H](C)COC(=O)[C@@H]3CCCN(N3)C2=O)C(C)C)C1] |
| BA216 | 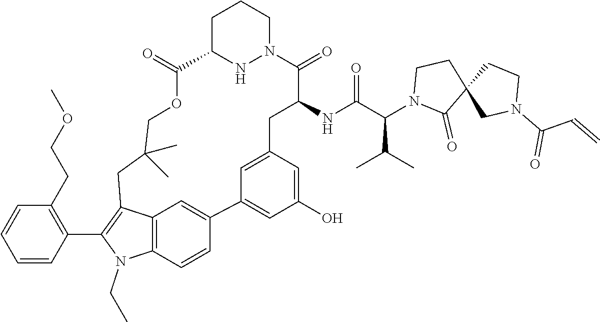 [SMILES: C=CC(=O)N1CC[C@]2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| BA217 | 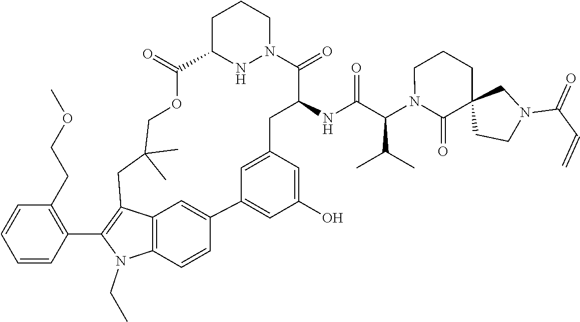 [SMILES: C=CC(=O)N1CC[C@]2(CCCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| BA218 | 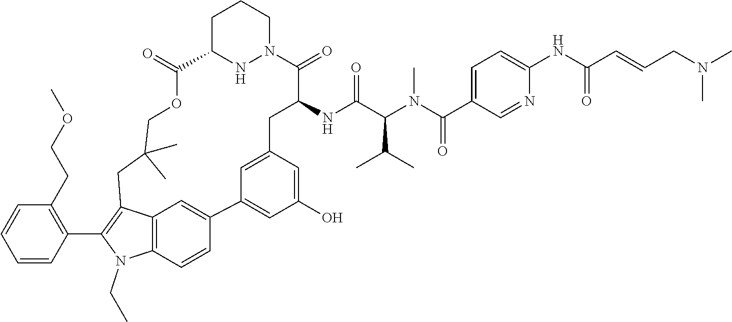 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)nc1)C(=O)N1CCC[C@H](N1)C(=O)OCC(C)(C)C2] |
| BA219 | 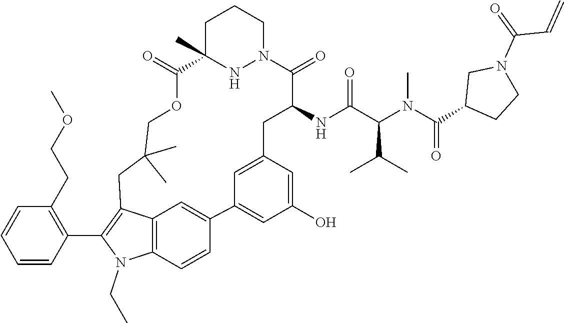 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@]3(C)CCCN(N3)C2=O)C(C)C)C1] |
| BA220 | 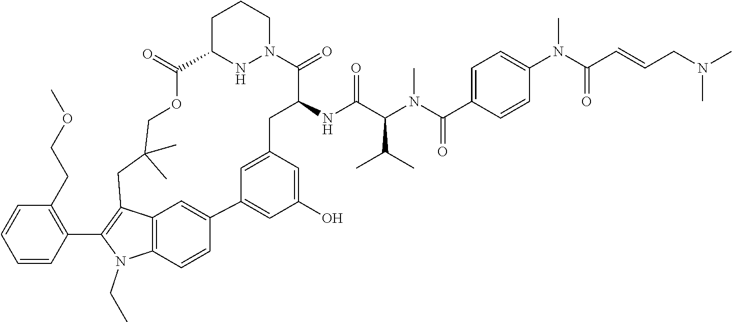 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(N(C)C(=O)/C=C/CN(C)C)cc1)C(=O)N1CCC[C@H](N1)C(=O)OCC(C)(C)C2] |
| BA221 | 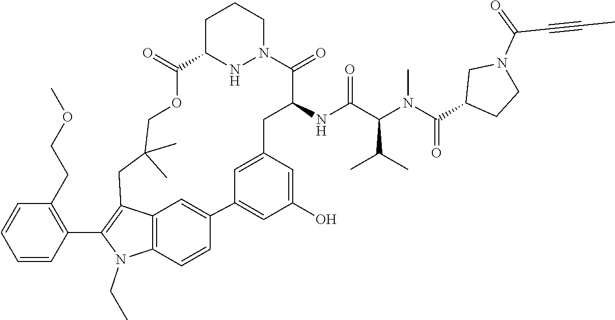 [SMILES: CC#CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA222 | 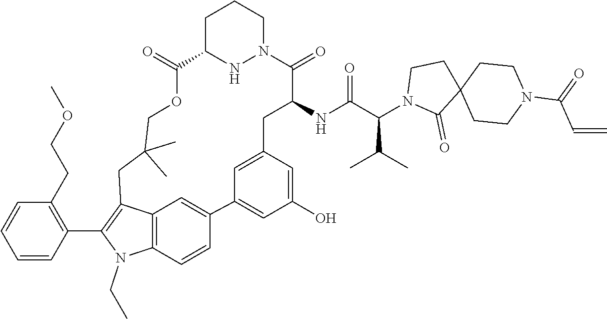 [SMILES: C=CC(=O)N1CCC2(CC1)CCN([C@H](C(=O)N[C@H]1Cc3cc(O)cc(c3)-c3ccc4c(c3)c(c(-c3ccccc3CCOC)n4CC)CC(C)(C)COC(=O)[C@@H]3CCCN(N3)C1=O)C(C)C)C2=O] |
| BA223 | 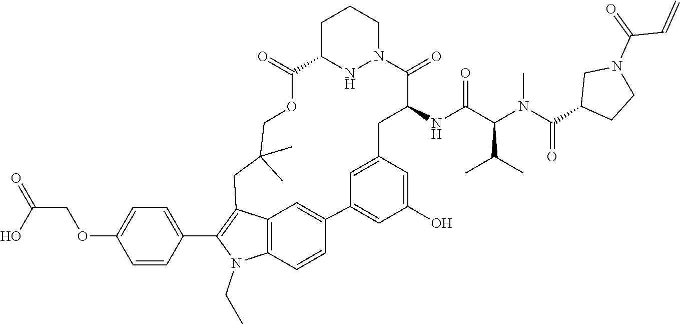 [SMILES: C=CC(=O)N1CC[C@H](C(=O)N(C)[C@H](C(=O)N[C@H]2Cc3cc(O)cc(c3)-c3ccc4c(c3)c(c(-c3ccc(OCC(=O)O)cc3)n4CC)CC(C)(C)COC(=O)[C@@H]3CCCN(N3)C2=O)C(C)C)C1] |
| BA224 | 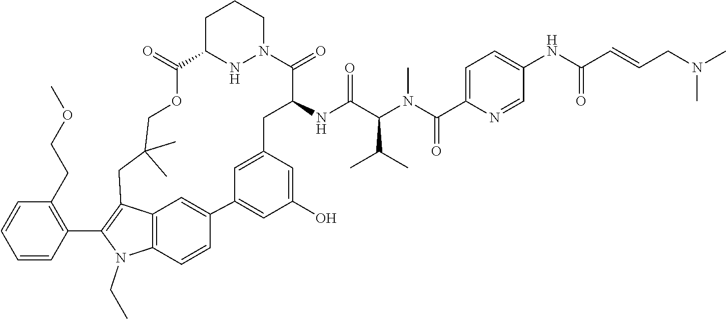 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| BA225 | 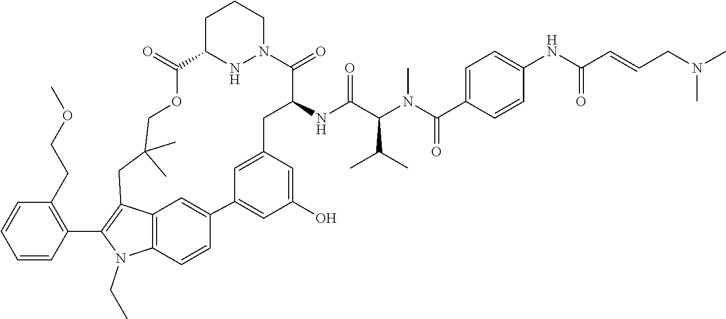 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cc1)C(=O)N1CCC[C@H](N1)C(=O)OCC(C)(C)C2] |
| BA226 | 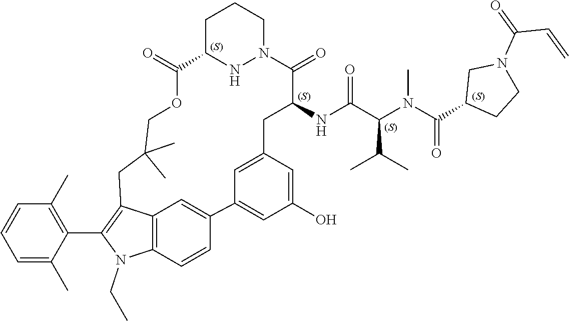 |
| BA227 | 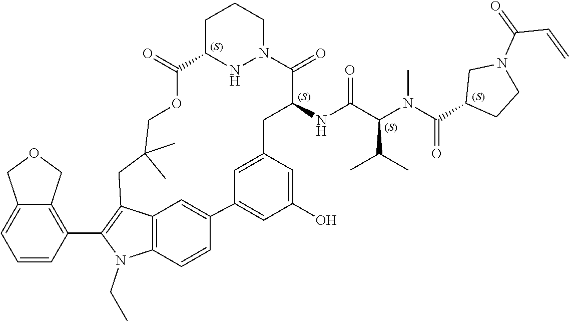 |
| BA228 | 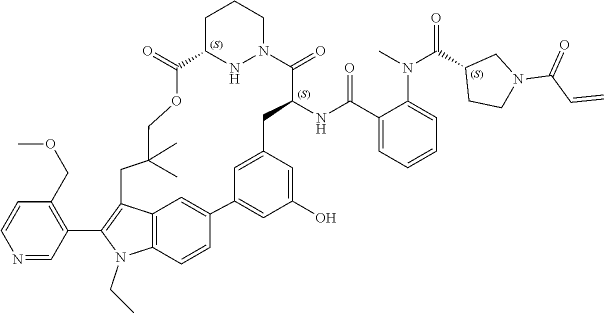 |
| BA229 | 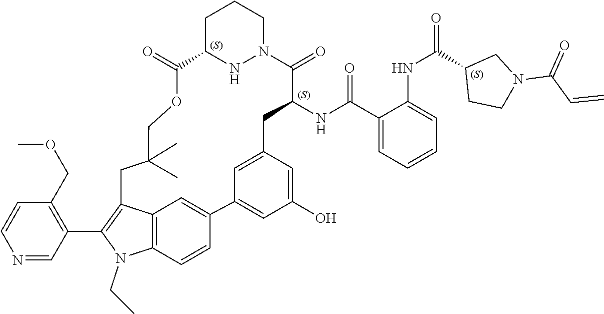 |
| BA230 | 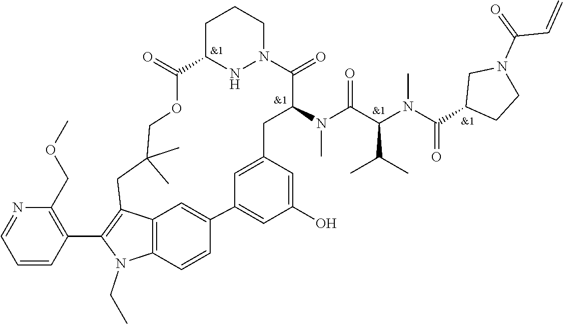 |
| BA231 | 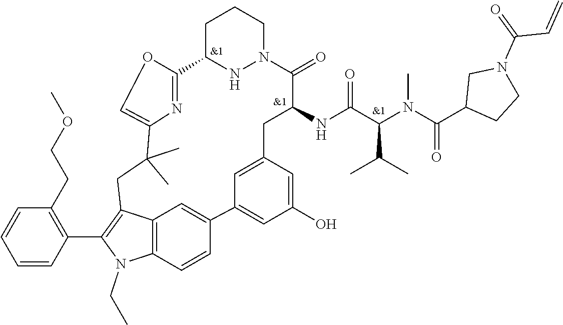 |
| BA232 | 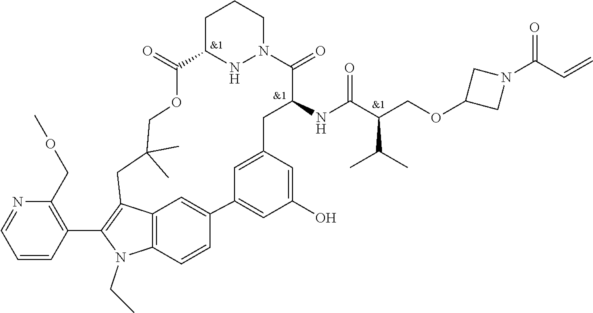 |
| BA233 | 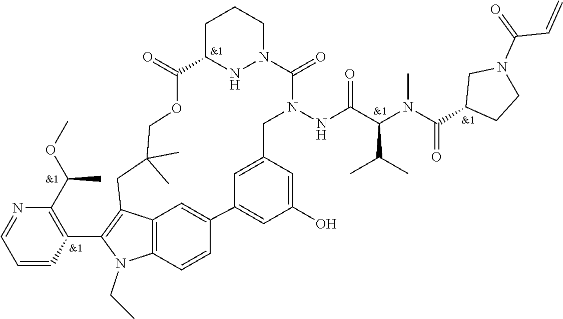 |
| BA234 | 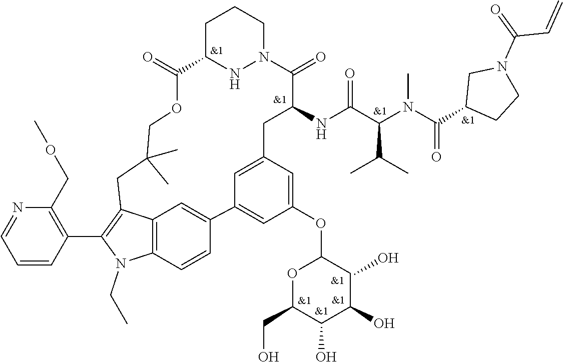 |
| BA235 | 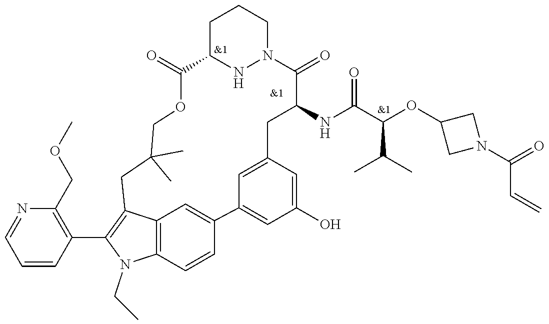 |
| BA236 | 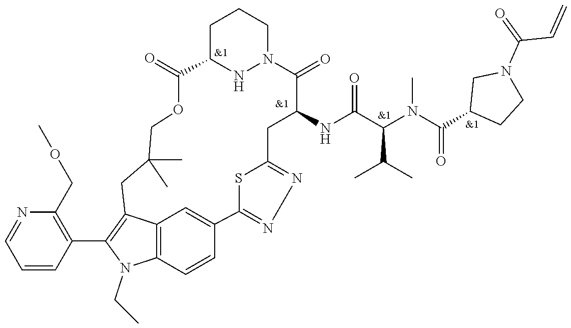 |
| BA237 | 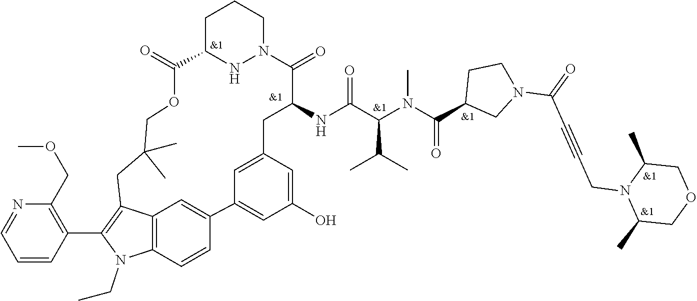 |
| BA238 | 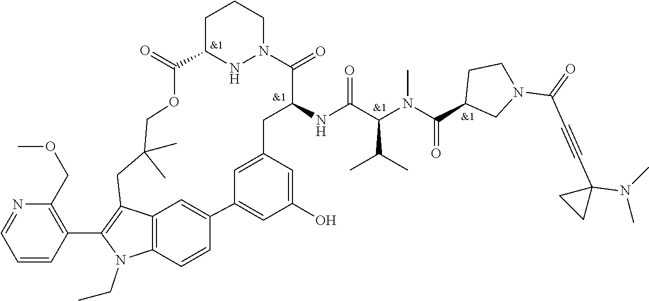 |
| BA239 | 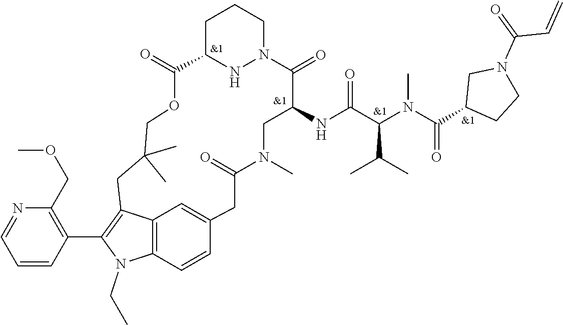 |
| BA240 | 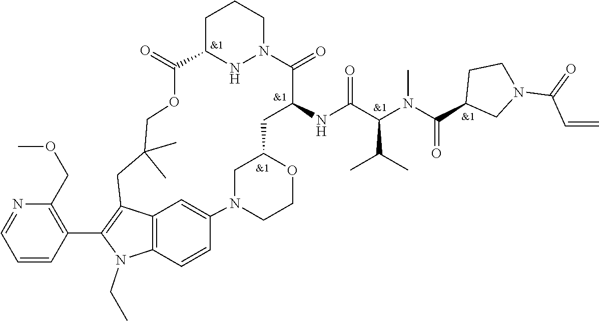 |
| BA241 | 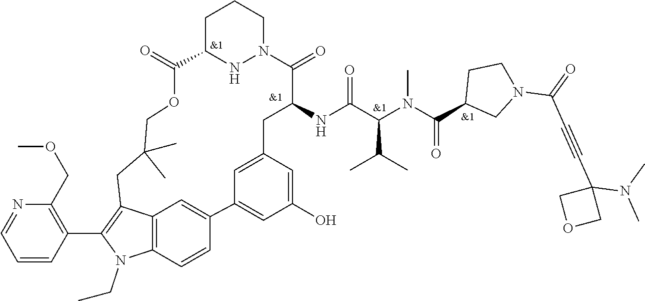 |
| BA242 | 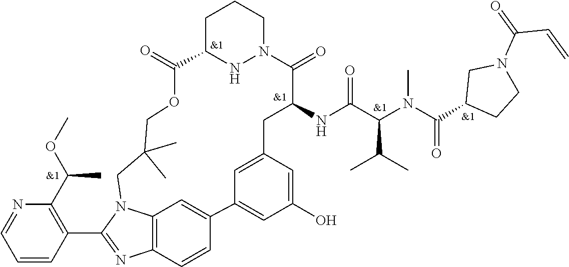 |
| BA243 | 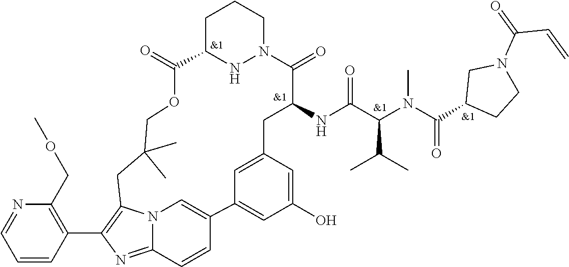 |
| BA244 | 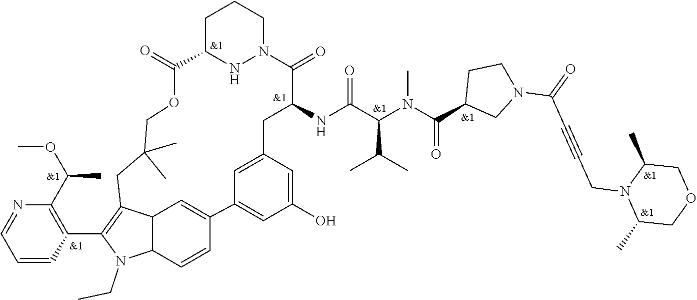 |
| BA245 | 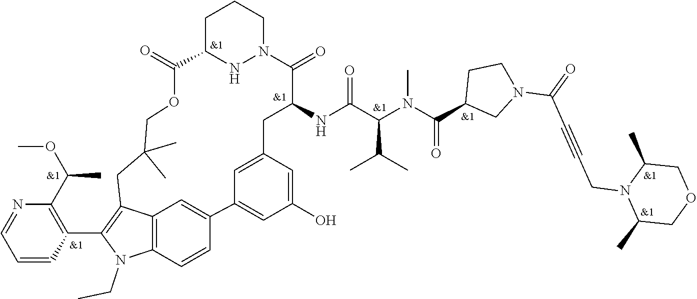 |
| BA246 | 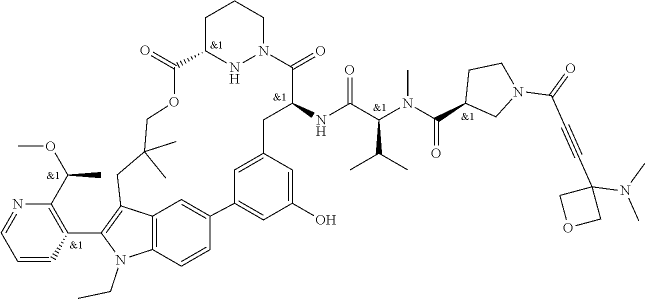 |
| BA247 | 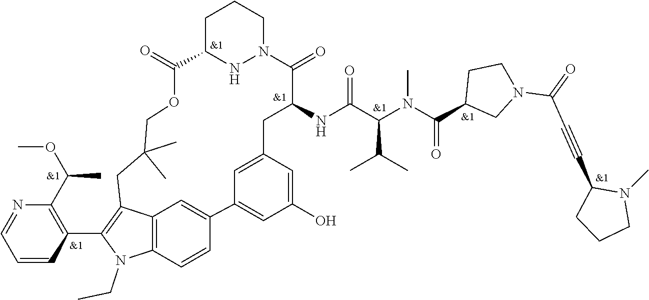 |
| BA248 | 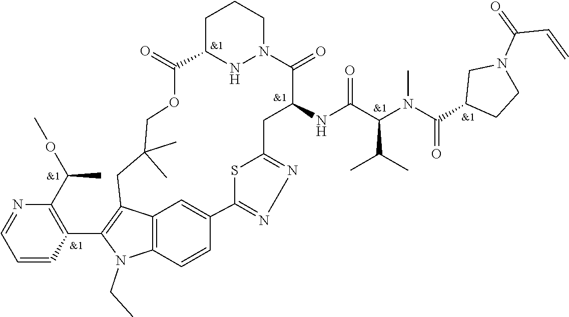 |
| BA249 | 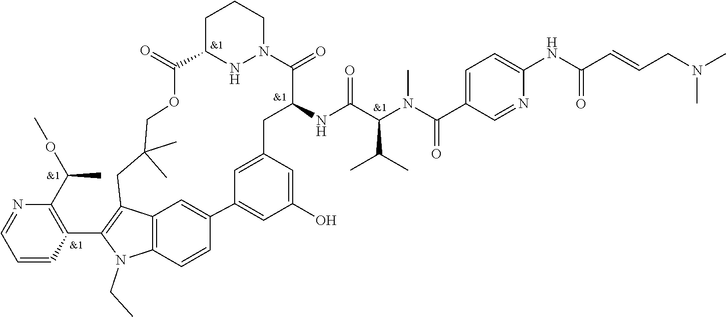 |
| BA250 | 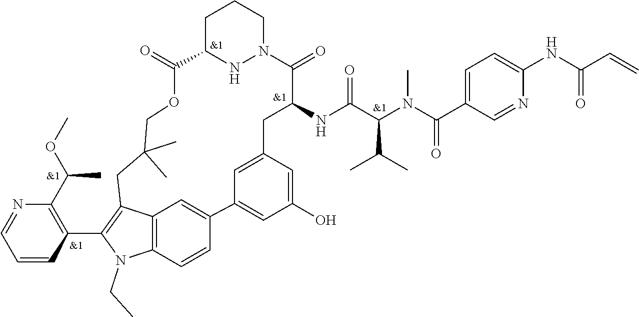 |
| BA251 | 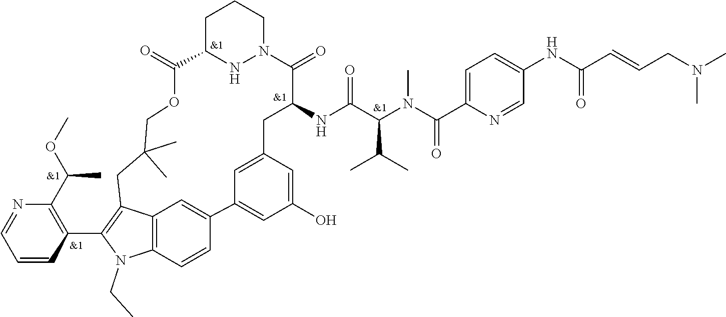 |
| BA252 | 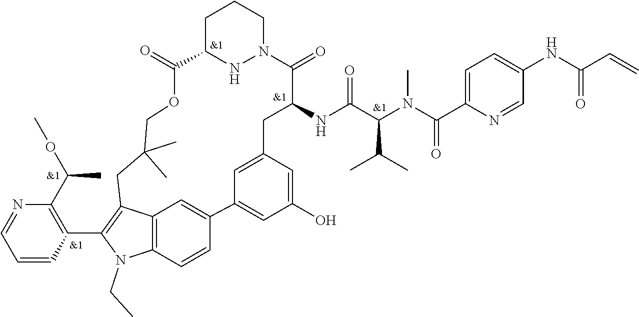 |
| BA253 | 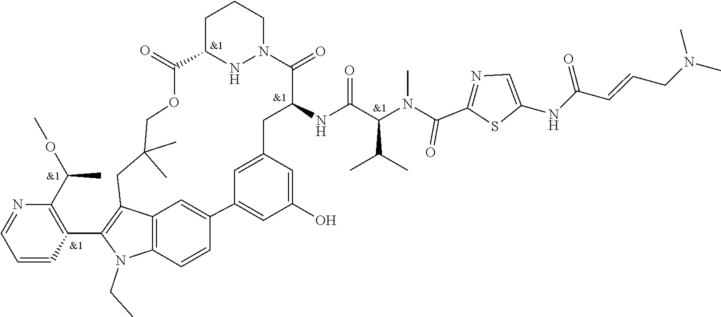 |
| BA254 | 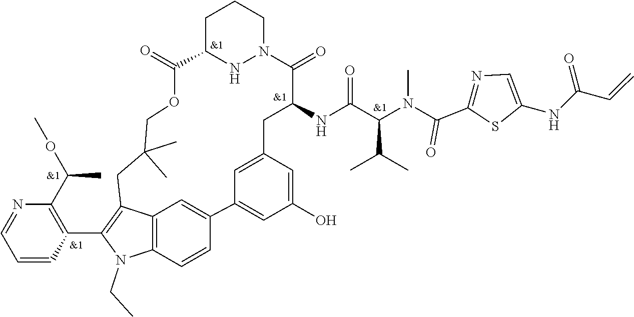 |
| BA255 | 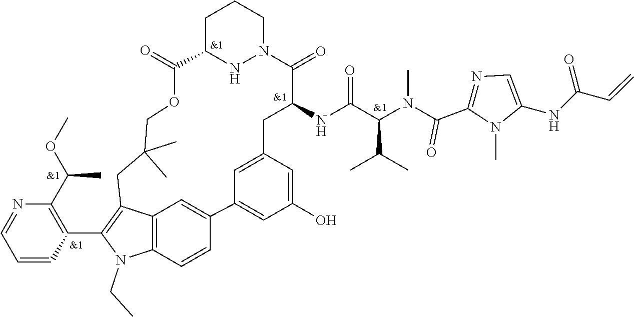 |
| BA256 | 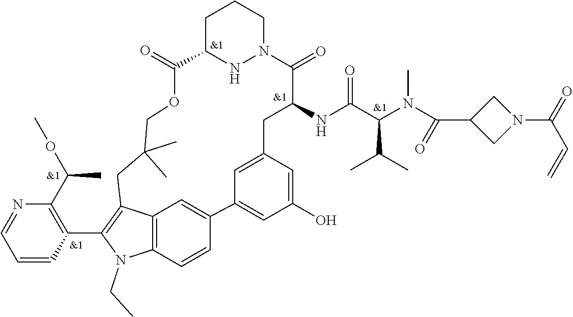 |
| BA257 | 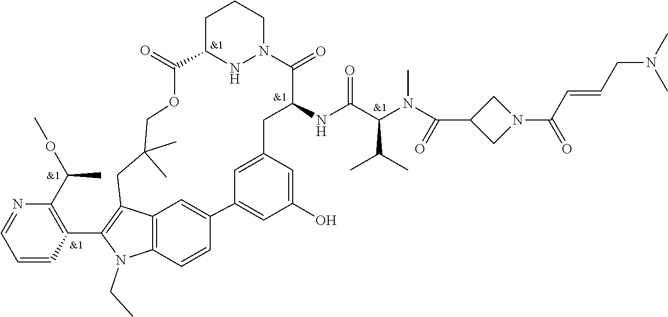 |
| BA258 | 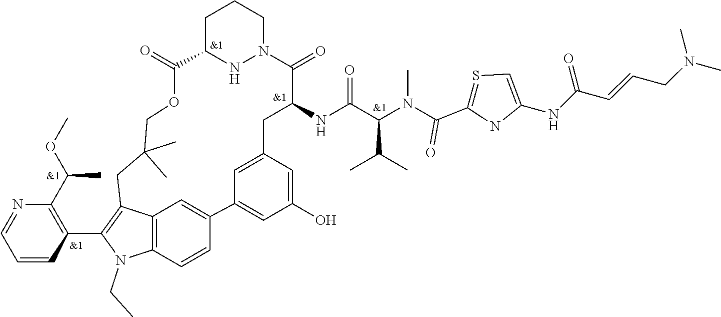 |
| BA259 | 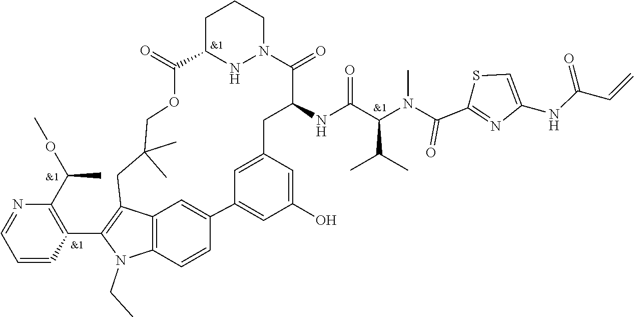 |
| BA260 | 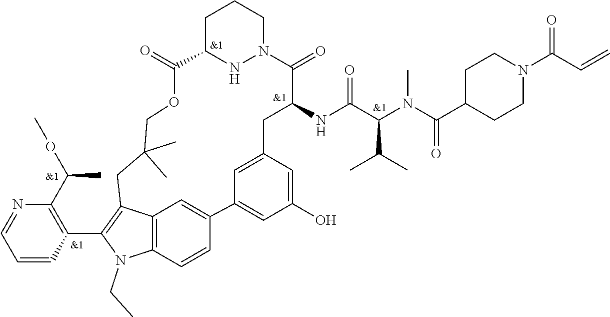 |
| BA261 | 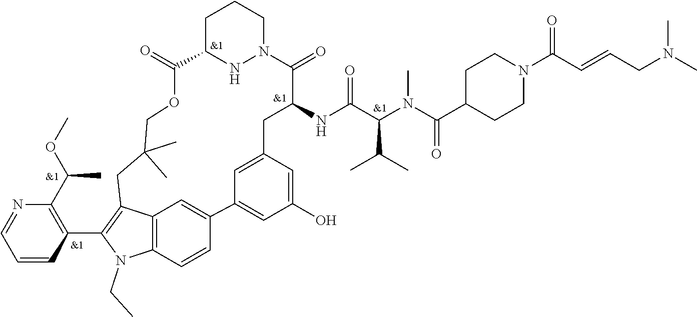 |
| BA262 | 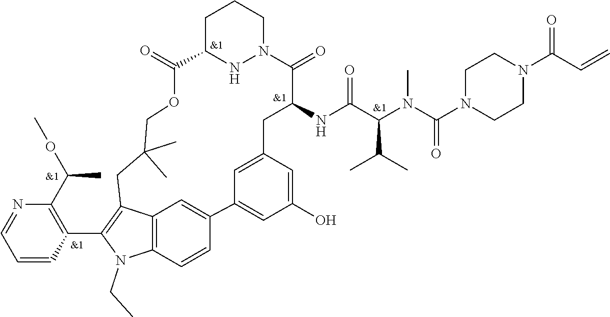 |
| BA263 | 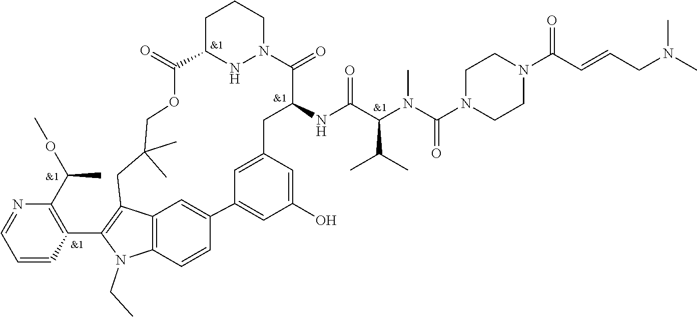 |
| BA264 | 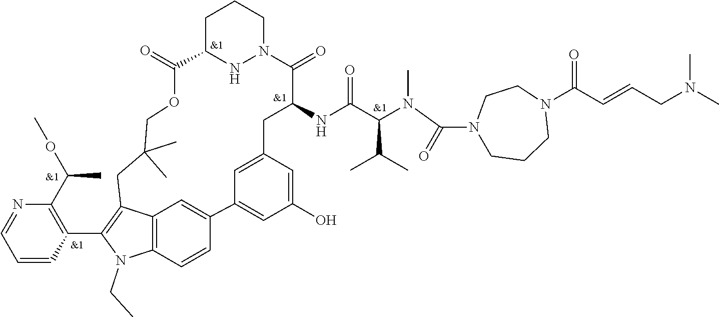 |
| BA265 | 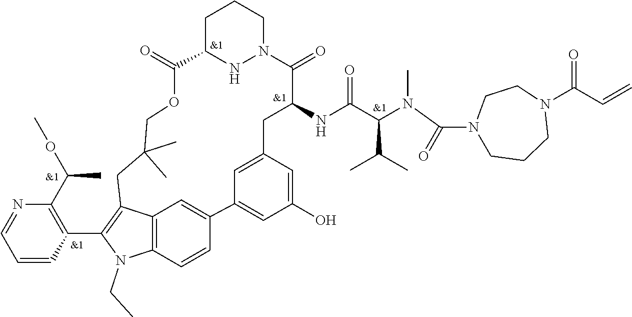 |
| BA266 | 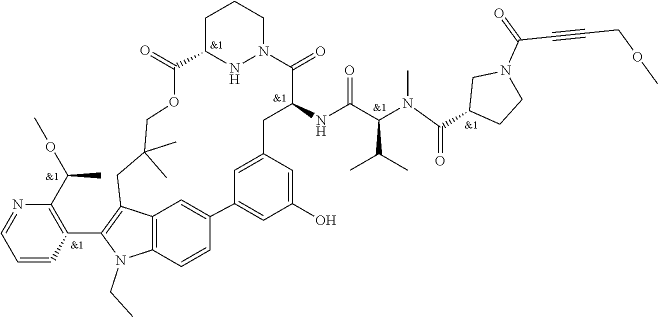 |
| BA267 | 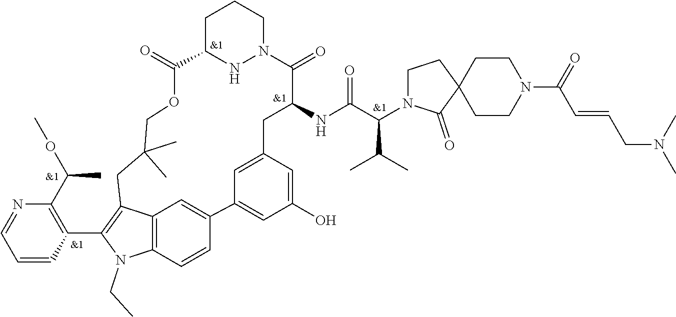 |
| BA268 | 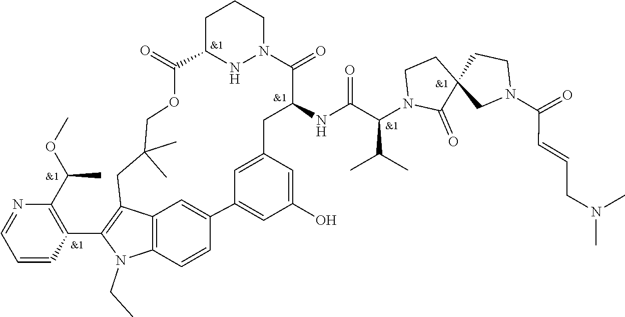 |
| BA269 | 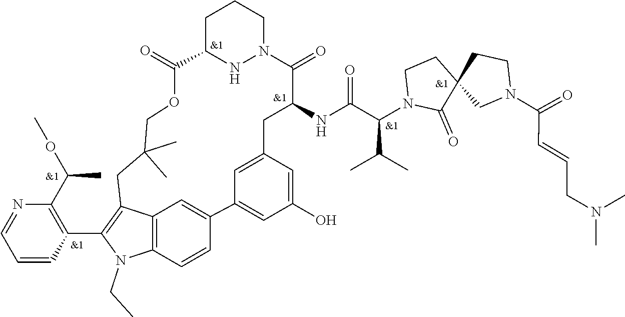 |
| BA270 | 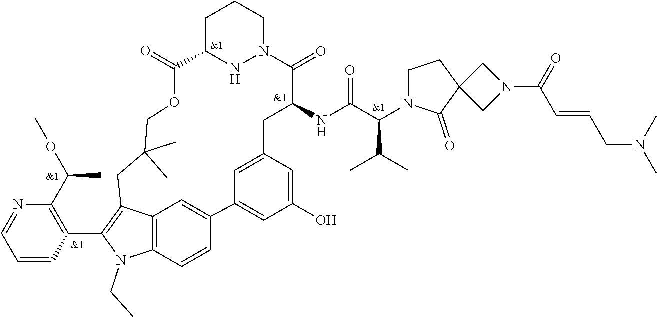 |
| BA271 | 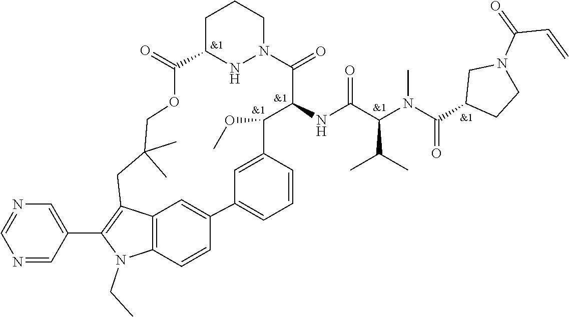 |
| BA272 | 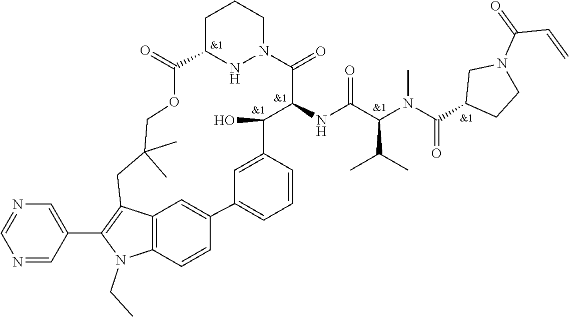 |
| BA273 | 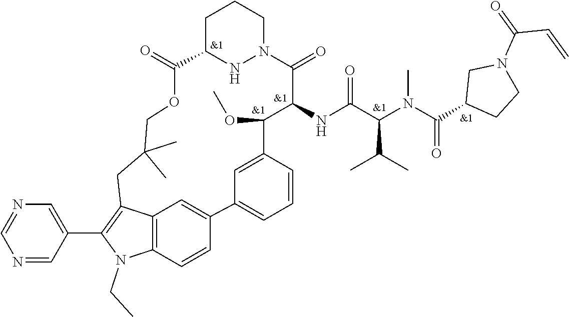 |
| BA274 | 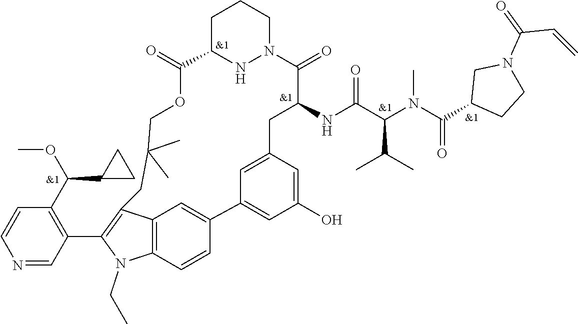 |
| BA275 | 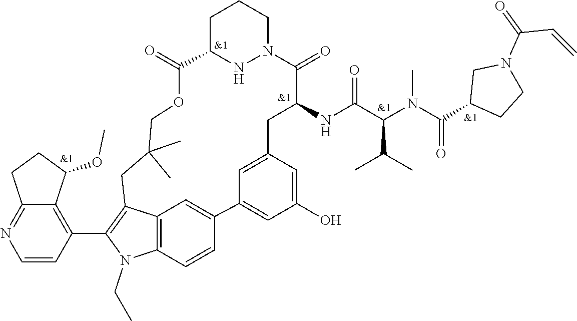 |
| BA276 | 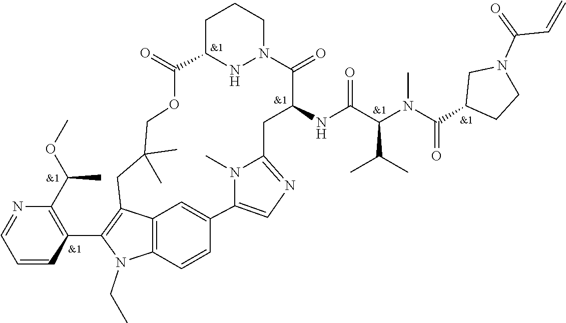 |
| BA277 | 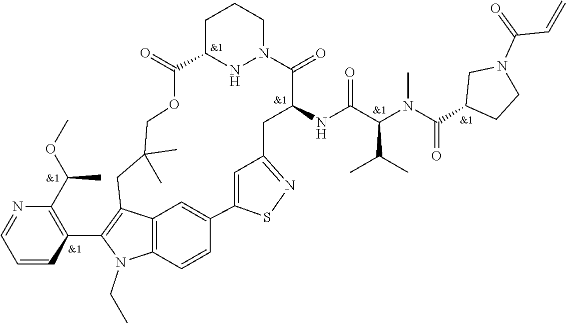 |
| BA278 | 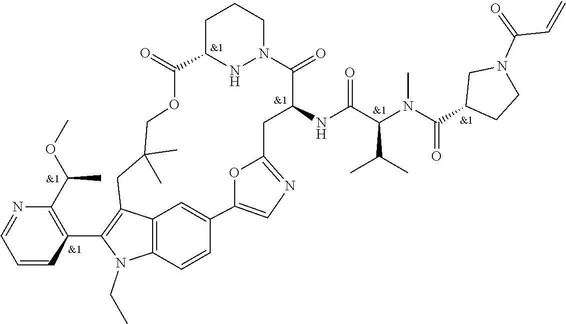 |
| BA279 | 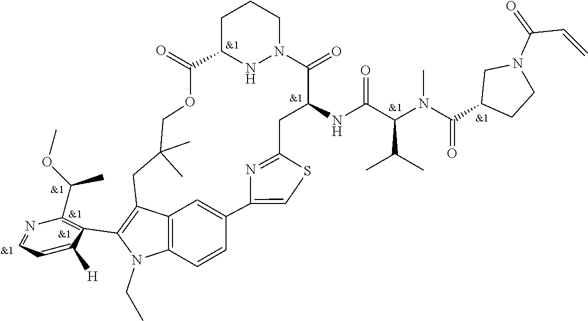 |
| BA280 | 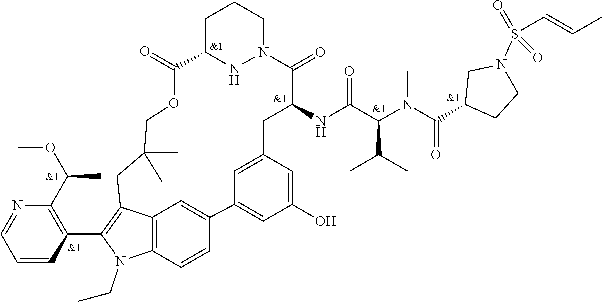 |
| BA281 | 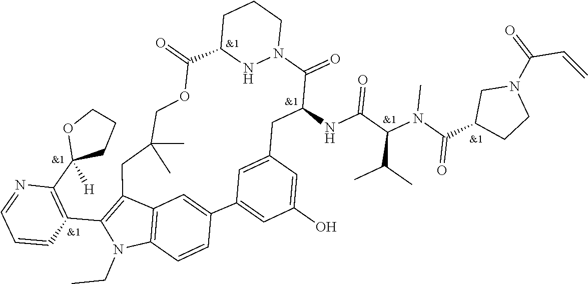 |
| BA282 | 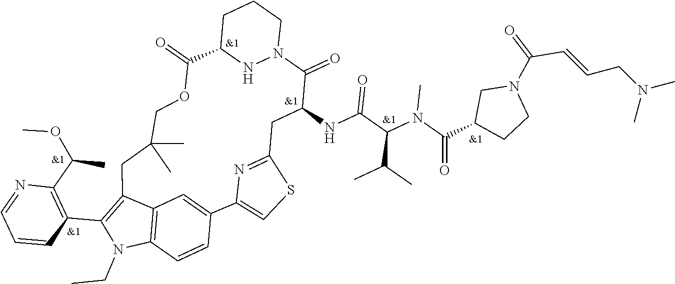 |
| BA283 | 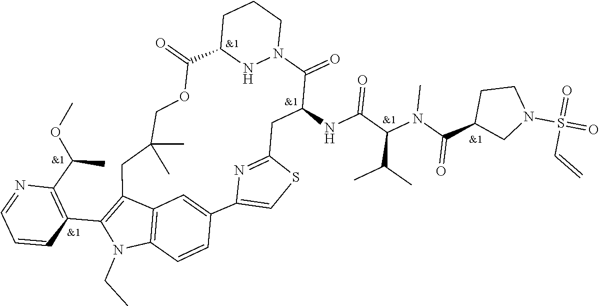 |
| BA284 | 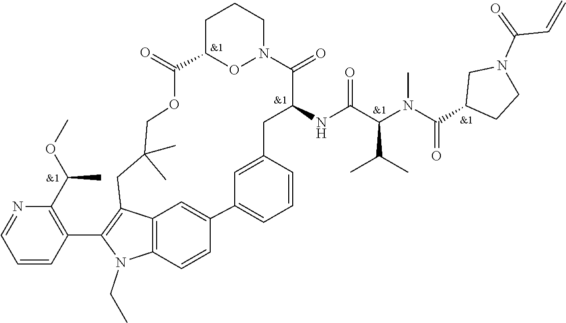 |
| BA285 | 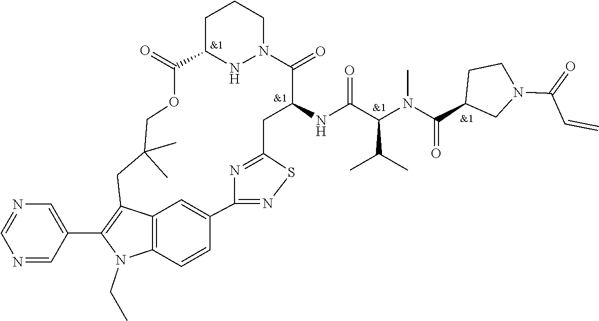 |
| BA286 | 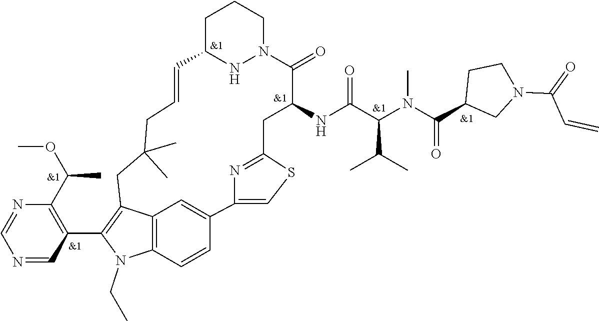 |
| BA287 | 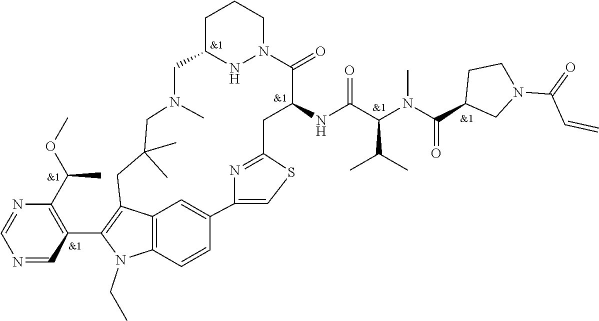 |
| BA288 | 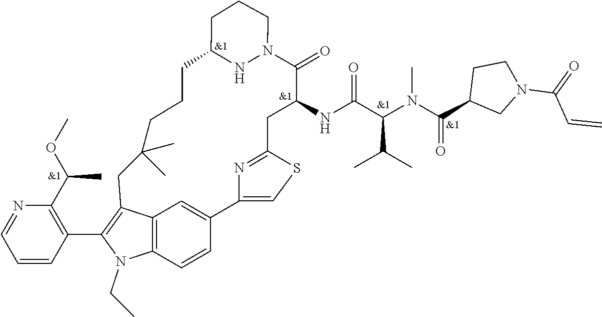 [SMILES: C=CC(=O)N1CC[C@H](C(=O)N(C)[C@H](C(=O)N[C@H]2Cc3nc(cs3)-c3ccc4c(c3)c(c(-c3cccnc3[C@H](C)OC)n4CC)CC(C)(C)CCC[C@@H]3CCCN(N3)C2=O)C(C)C)C1] |
| BA289 | 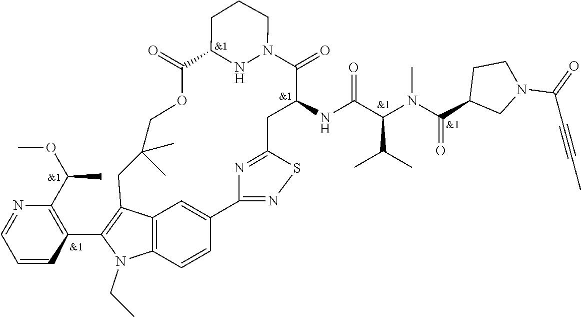 |
| BA290 | 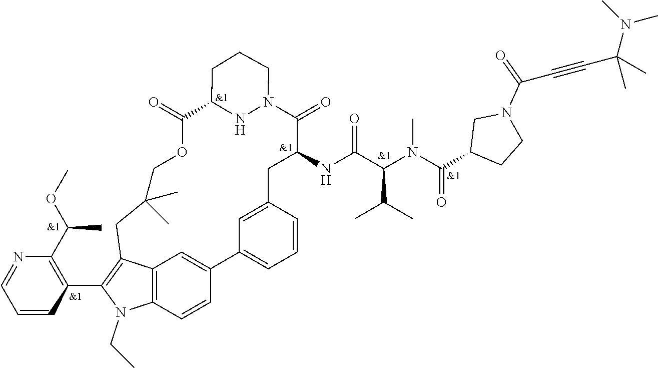 |
| BA291 | 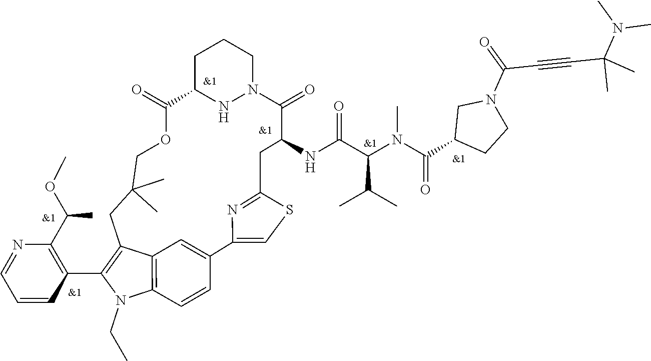 |
| BA292 | 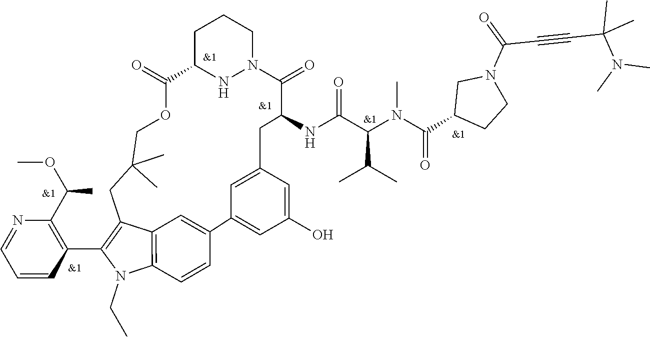 |
| BA293 | 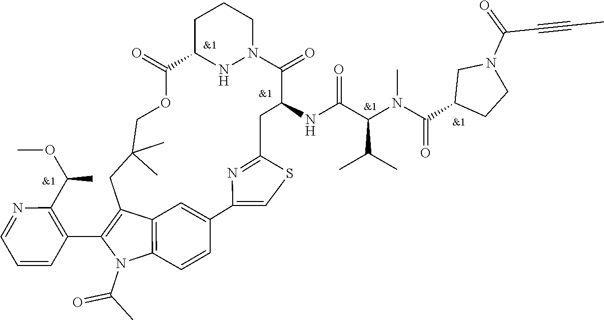 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(C)=O)CC(C)(C)COC(=O)[C@@H]3CCCN(N3)C2=O)C(C)C)C1] |
| BA294 | 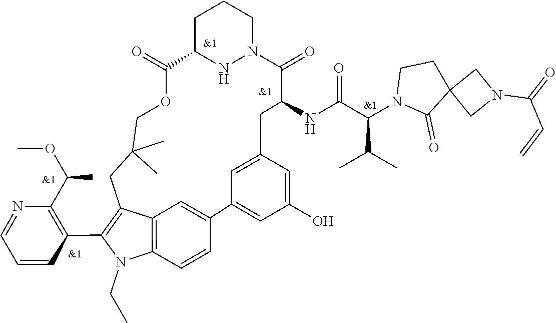 |
| BA295 | 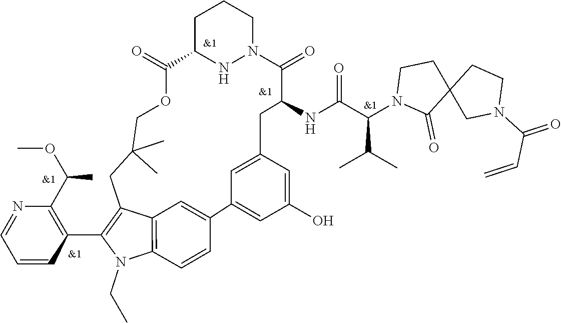 |
| BA296 | 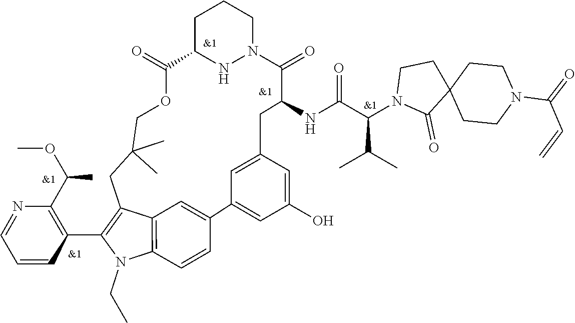 |
| BA297 | 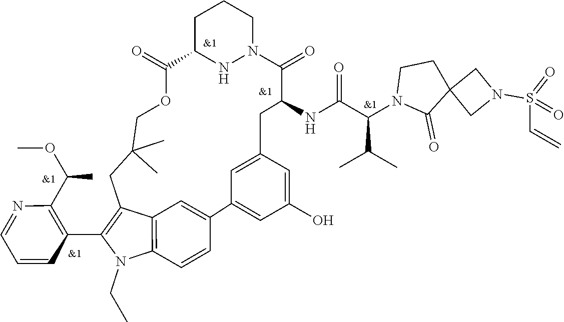 |
| BA298 | 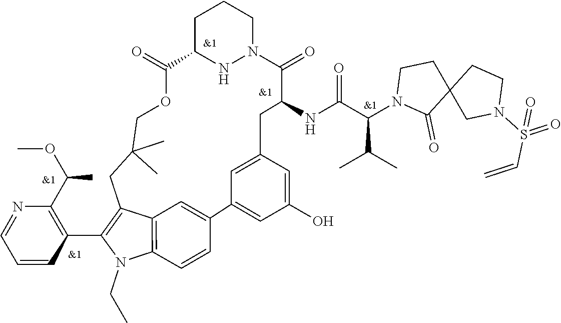 |
| BA299 | 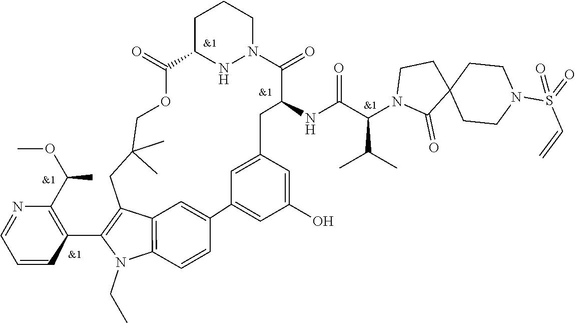 |
| BA300 | 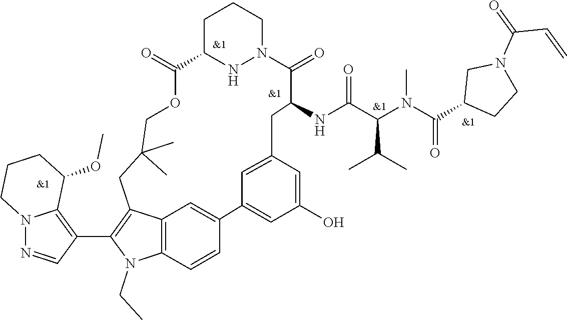 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| BA301 | 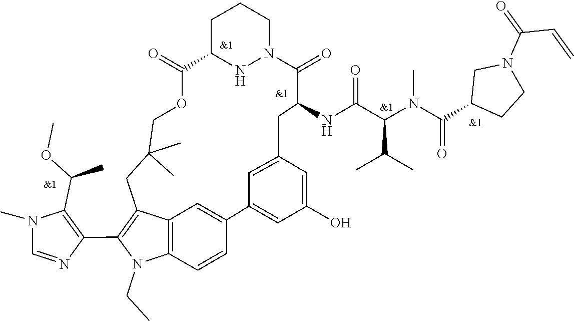 [SMILES: C=CC(=O)N1CC[C@H](C(=O)N(C)[C@H](C(=O)N[C@H]2Cc3cc(O)cc(c3)-c3ccc4c(c3)c(c(-c3ncn(C)c3[C@H](C)OC)n4CC)CC(C)(C)COC(=O)[C@@H]3CCCN(N3)C2=O)C(C)C)C1] |
| BA302 | 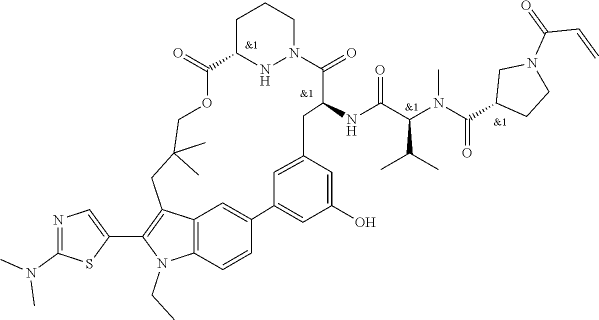 [SMILES: C=CC(=O)N1CC[C@H](C(=O)N(C)[C@H](C(=O)N[C@H]2Cc3cc(O)cc(c3)-c3ccc4c(c3)c(c(-c3cnc(N(C)C)s3)n4CC)CC(C)(C)COC(=O)[C@@H]3CCCN(N3)C2=O)C(C)C)C1] |
| BA303 | 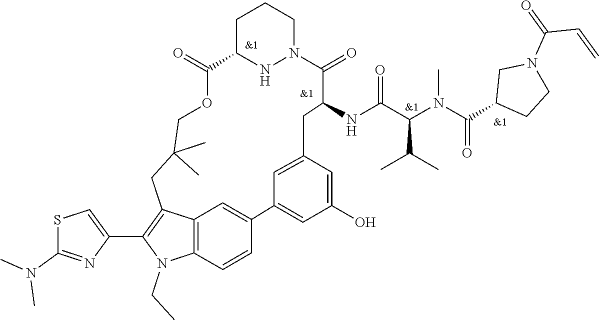 [SMILES: C=CC(=O)N1CC[C@H](C(=O)N(C)[C@H](C(=O)N[C@H]2Cc3cc(O)cc(c3)-c3ccc4c(c3)c(c(-c3csc(N(C)C)n3)n4CC)CC(C)(C)COC(=O)[C@@H]3CCCN(N3)C2=O)C(C)C)C1] |
| BA304 | 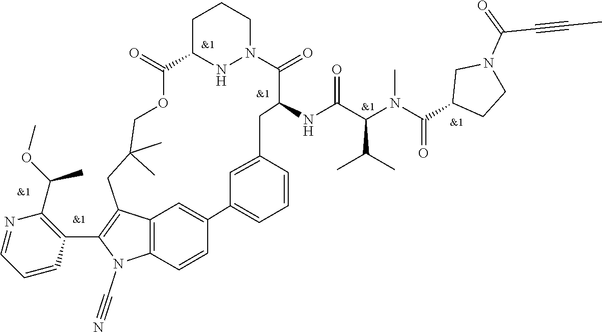 |
| BA305 | 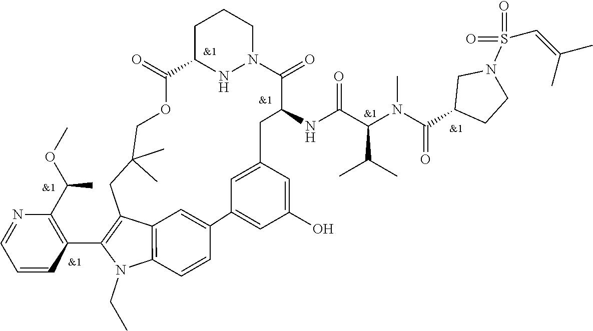 |
| BA306 | 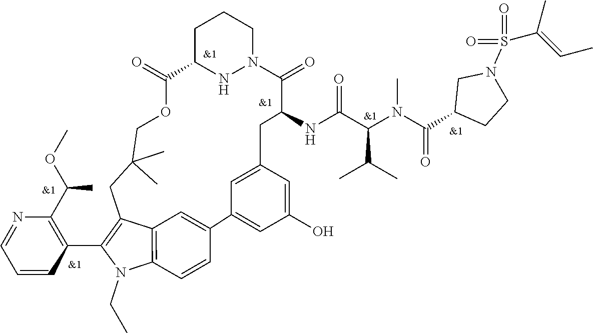 |
| BA307 | 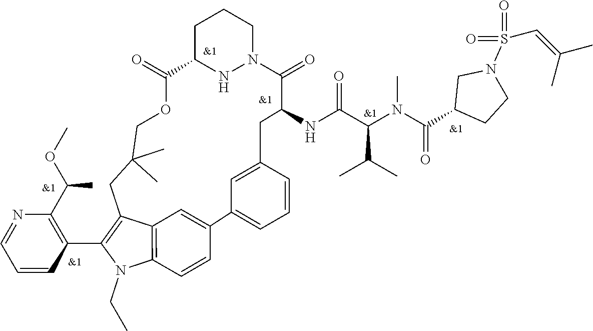 |
| BA308 | 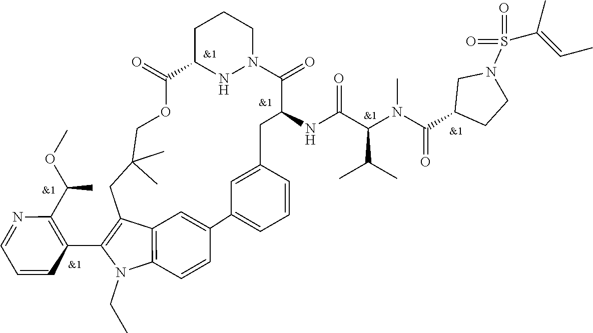 |
| BA309 | 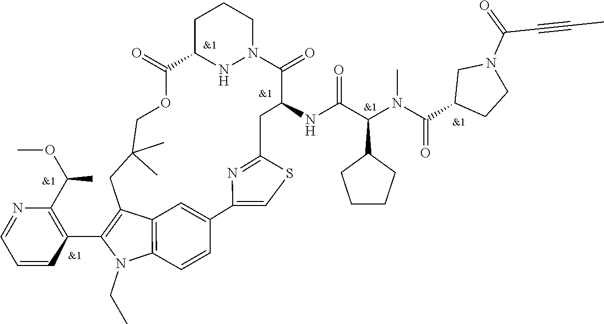 |
| BA310 | 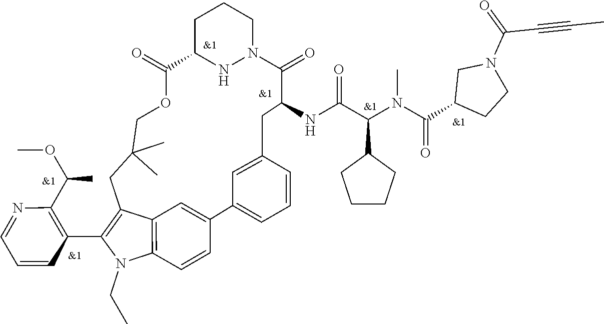 |
| BA311 | 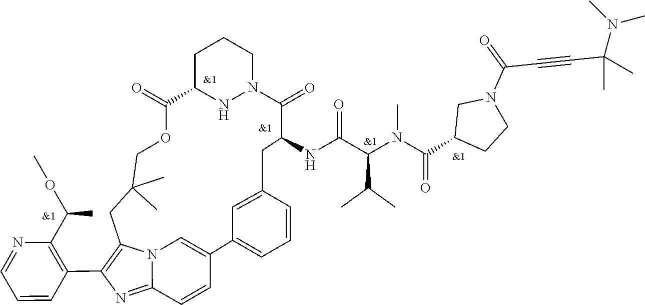 [SMILES: CO[C@@H](C)c1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CC(C)(C)N(C)C)C1)Cc1cccc-3c1] |
| BA312 | 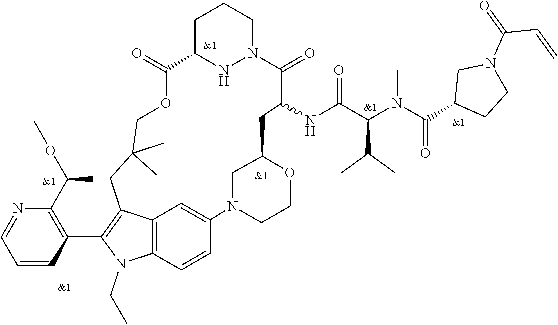 |
| BA313 | 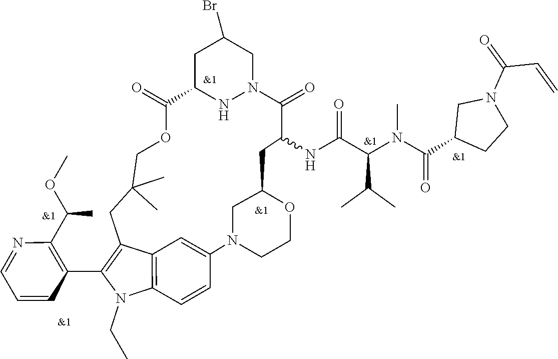 |
| BA314 | 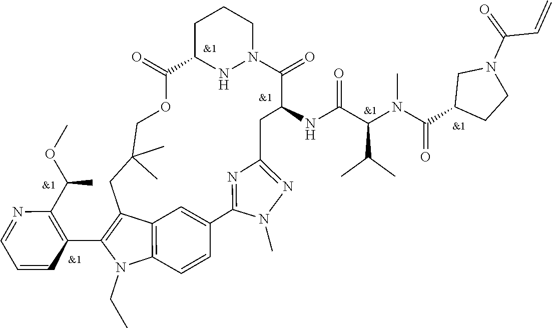 |
| BA316 | 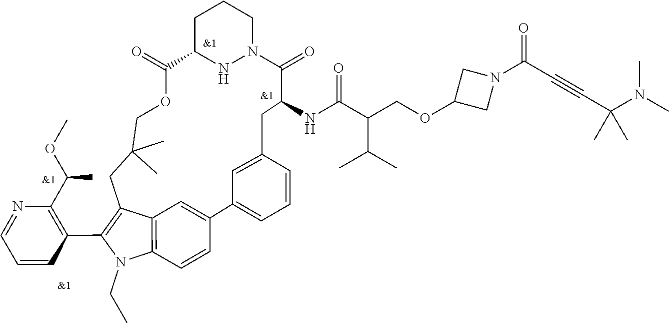 |
| BA317 | 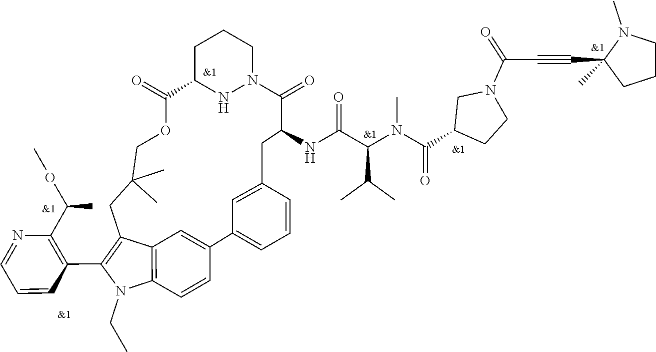 |
| BA318 | 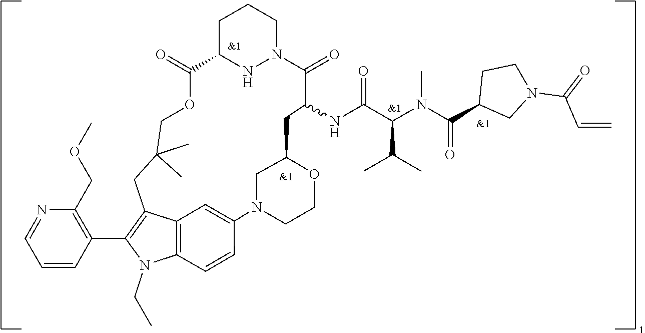 |
| BA319 | 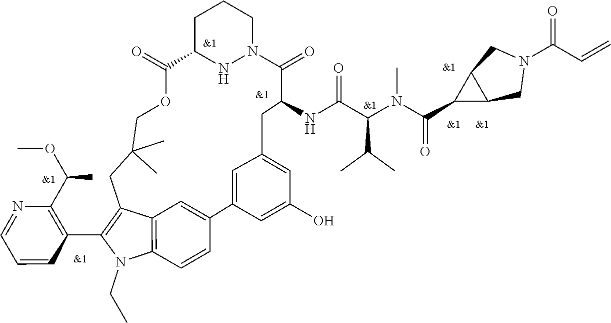 |
| BA320 | 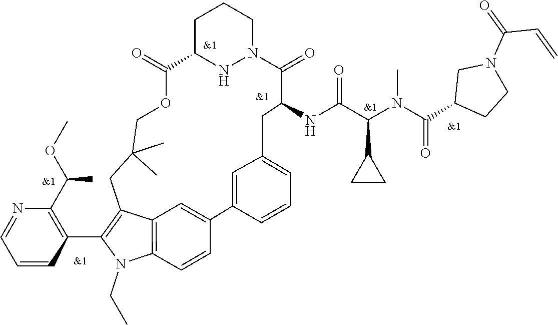 |
| BA321 | 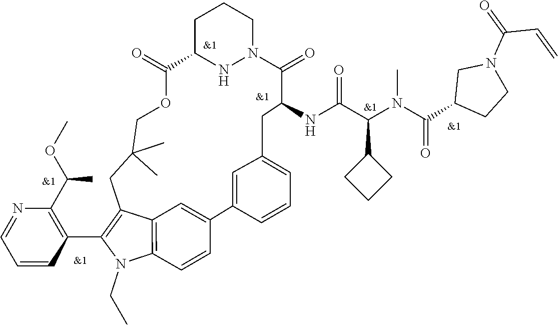 |
| BA322 | 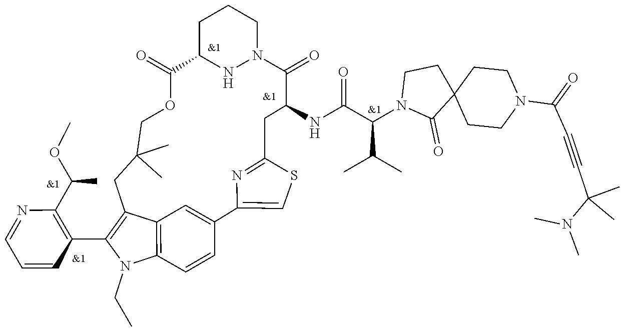 |
| BA323 | 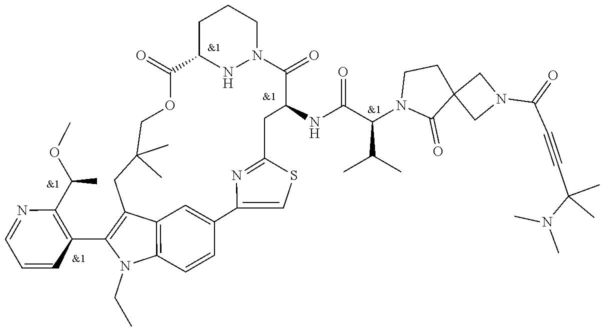 |
| BA324 | 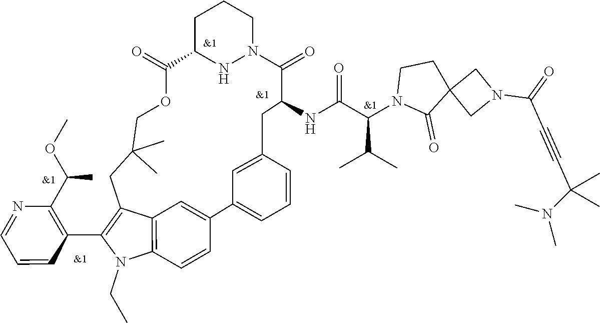 |
| BA325 | 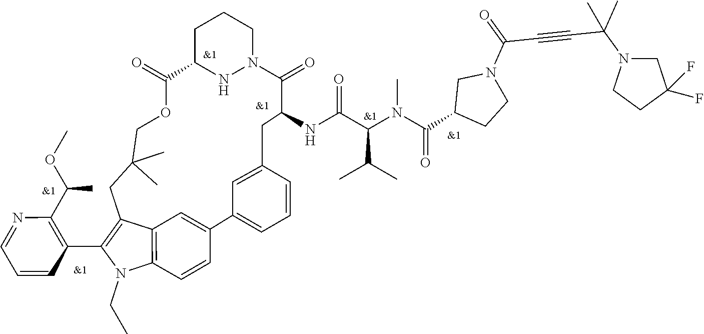 |
| BA326 | 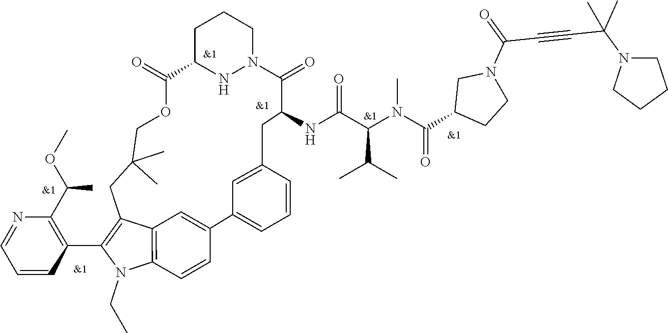 |
| BA327 | 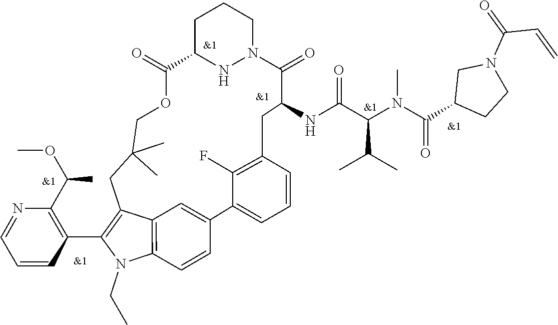 |
| BA328 | 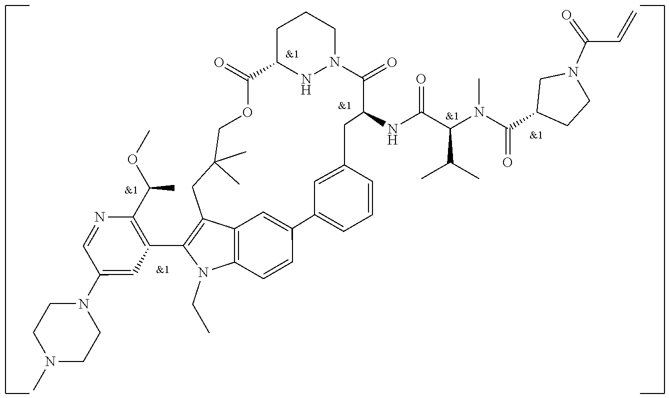 |
| BA329 | 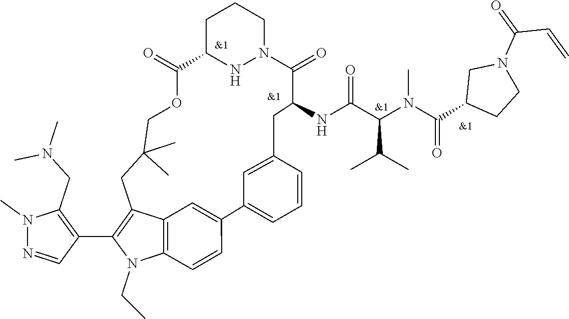 [SMILES: C=CC(=O)N1CC[C@H](C(=O)N(C)[C@H](C(=O)N[C@H]2Cc3cccc(c3)-c3ccc4c(c3)c(c(-c3cnn(C)c3CN(C)C)n4CC)CC(C)(C)COC(=O)[C@@H]3CCCN(N3)C2=O)C(C)C)C1] |
| BA330 | 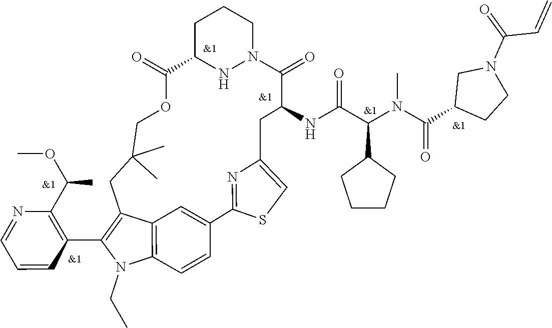 |
| BA331 | 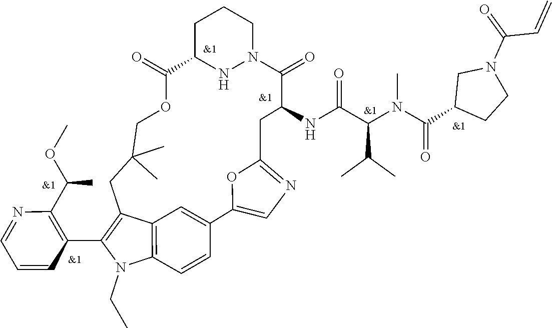 |
| BA332 | 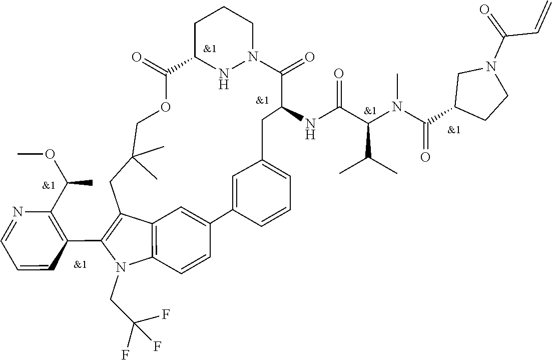 |
| BA333 | 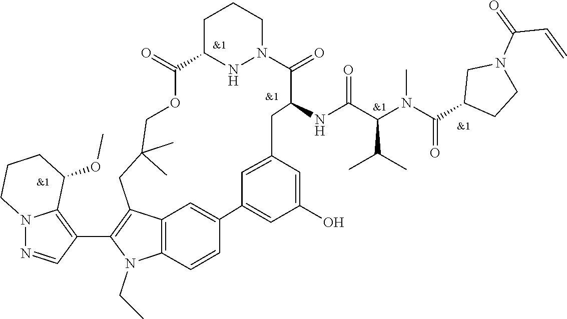 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| BA334 | 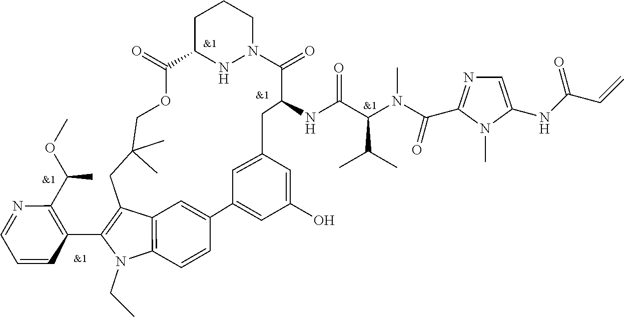 |
| BA335 | 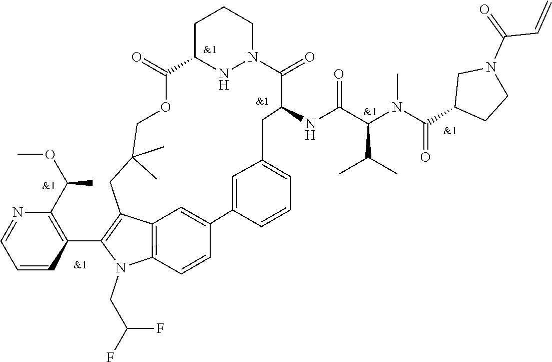 |
| BA336 | 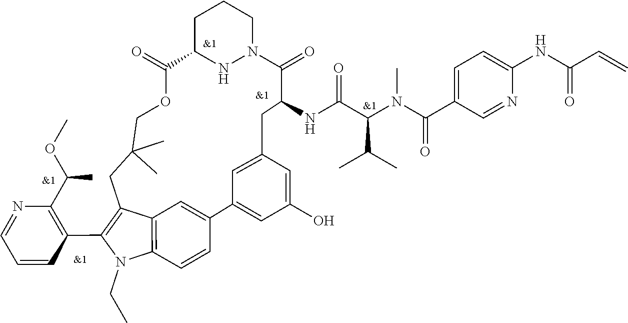 |
| BA337 | 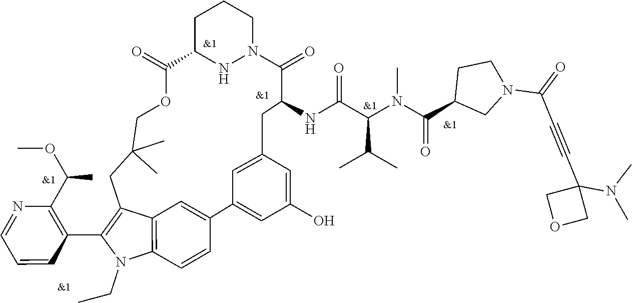 |
| BA338 | 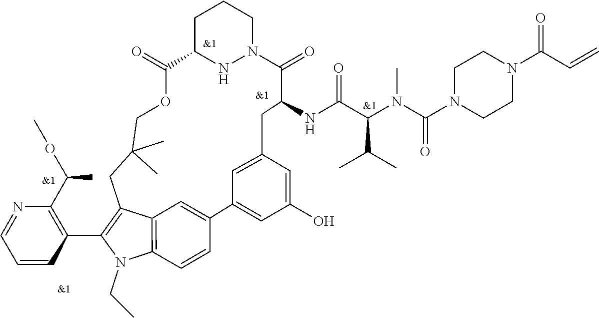 |
| BA339 | 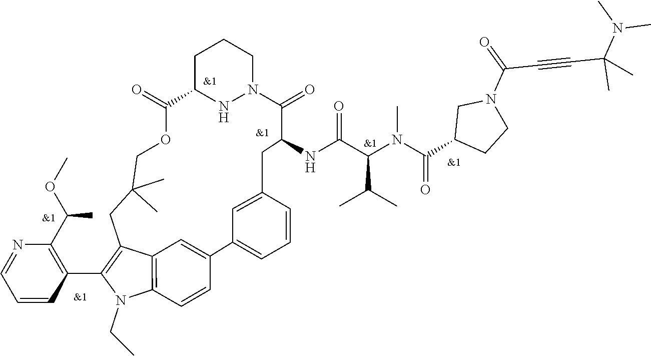 |
| BA340 | 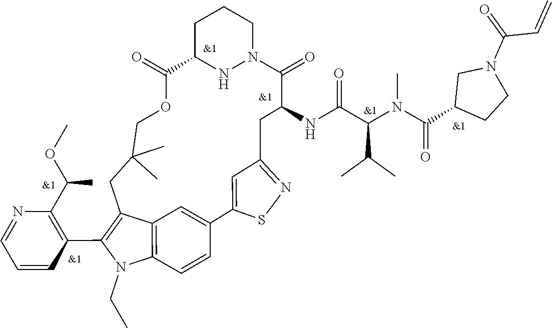 |
| BA341 | 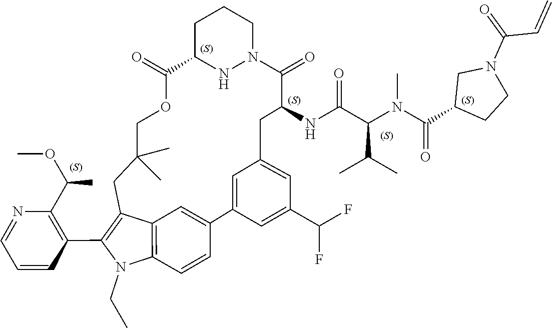 |
| BA342 | 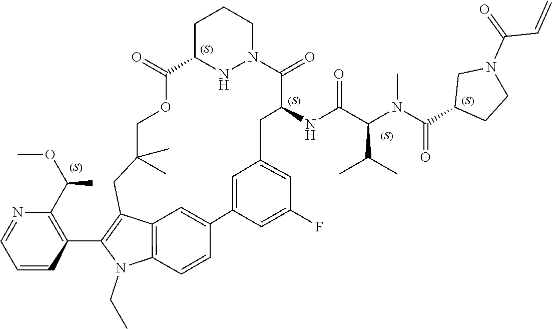 |
| BA343 | 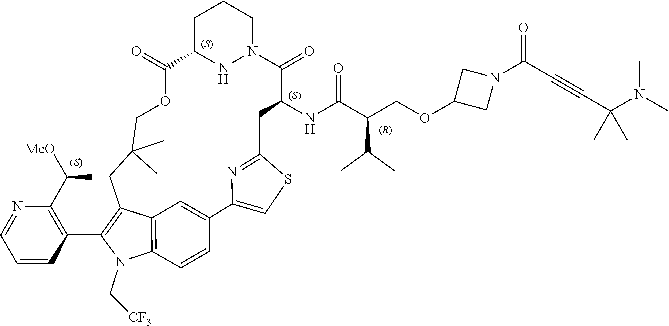 |
| BA344 | 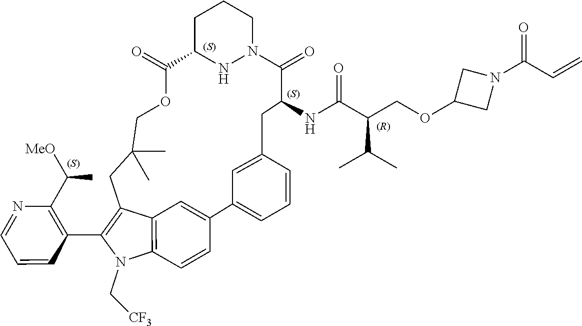 |
| BA345 | 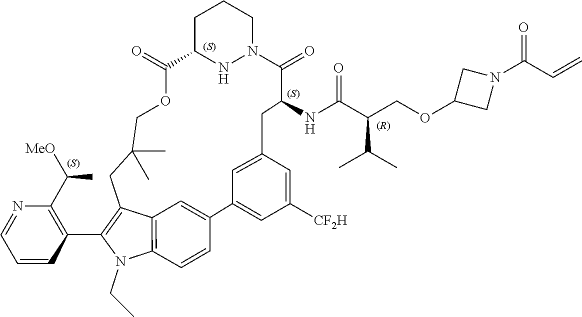 |
| BA346 | 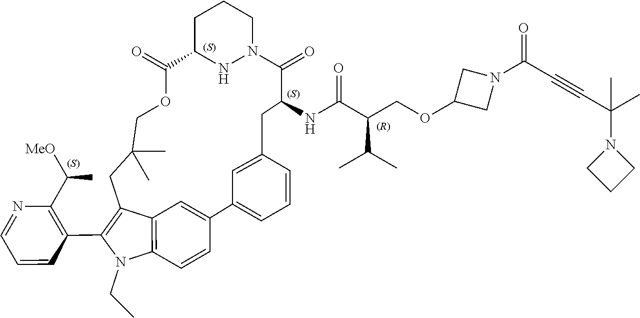 |
| BA347 | 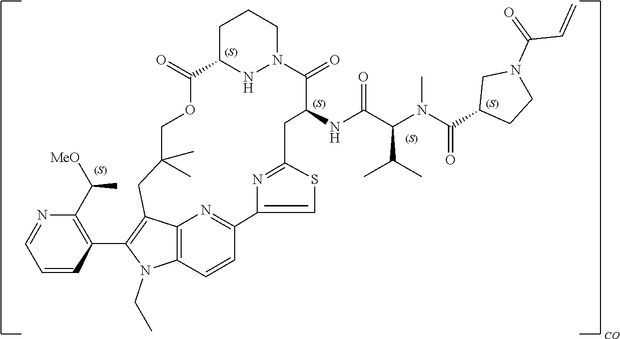 |
| BA348 | 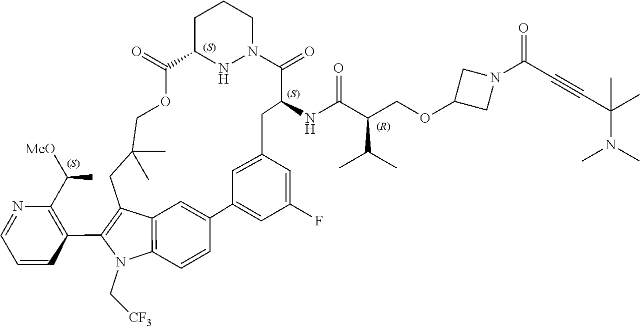 |
| BA349 | 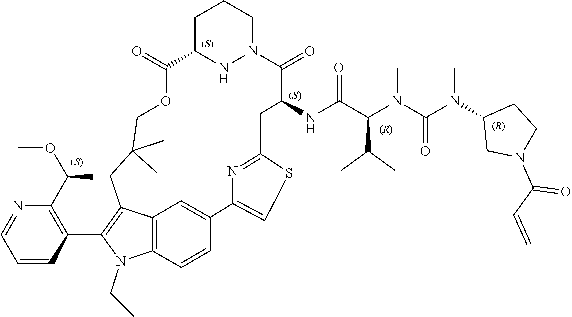 |
| BA350 | 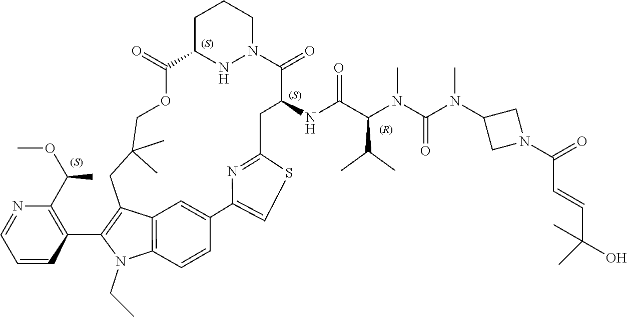 |
| BA351 | 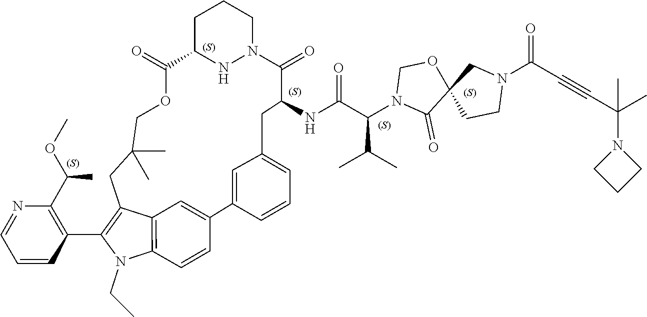 |
| BA352 | 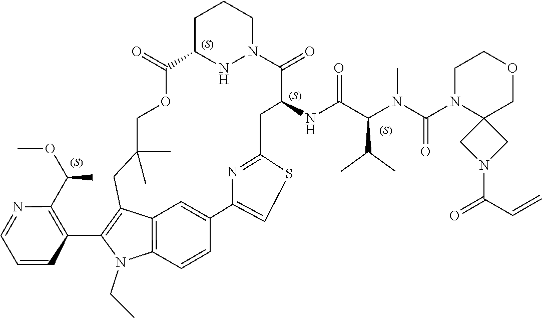 |
| BA353 | 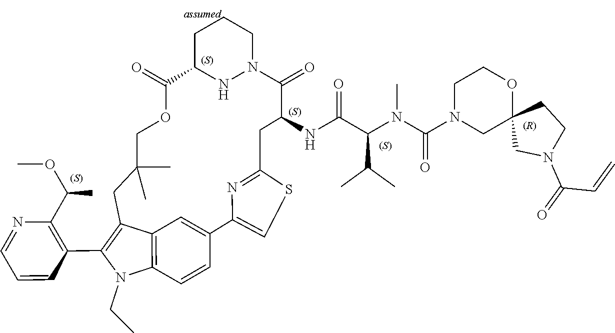 |
| BA354 | 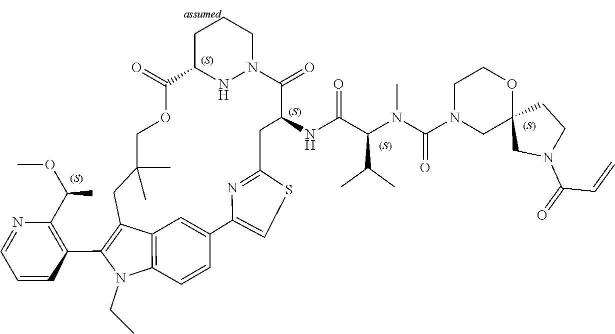 |
| BA355 | 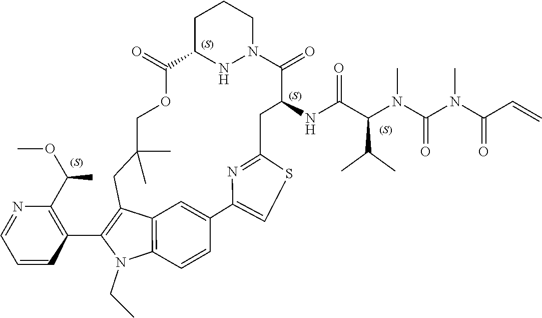 |
| BA356 | 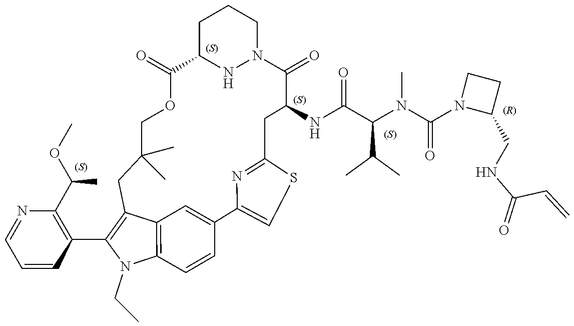 |
| BA357 | 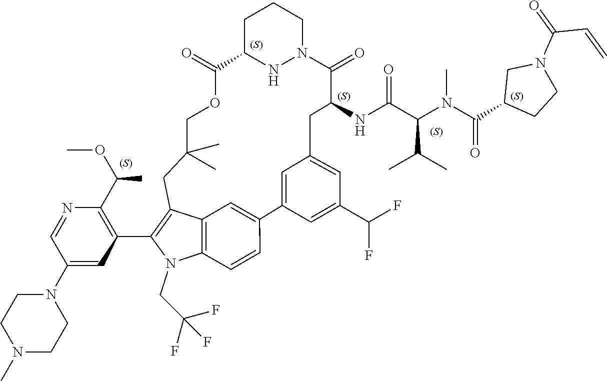 |
| BA358 | 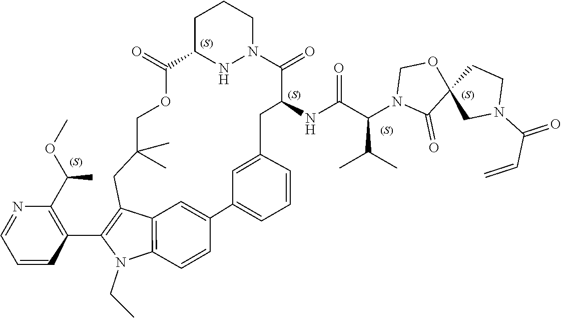 |
| BA359 | 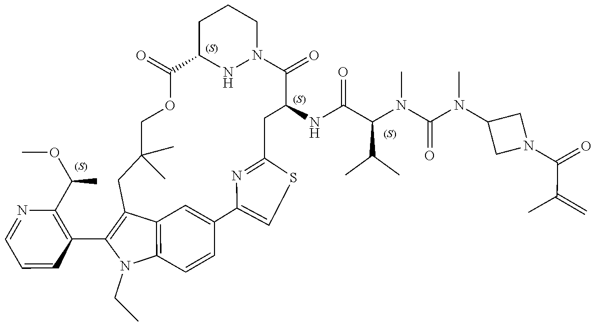 |
| BA360 | 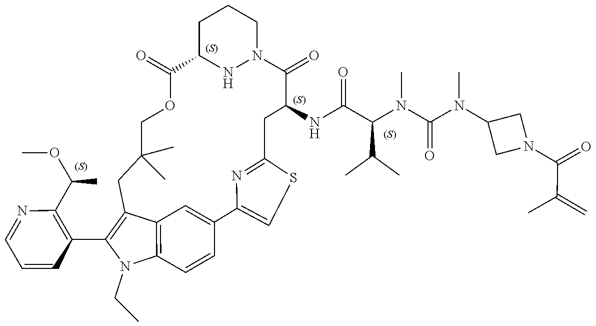 |
| BA361 | 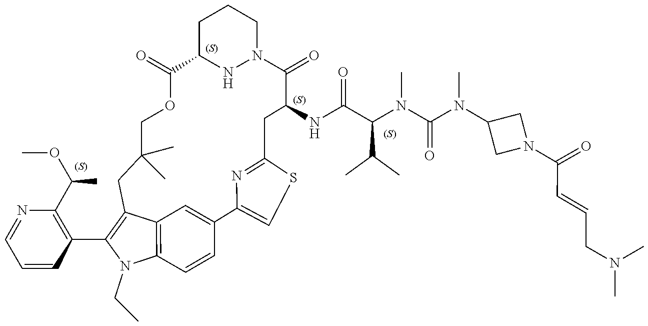 |
| BA362 | 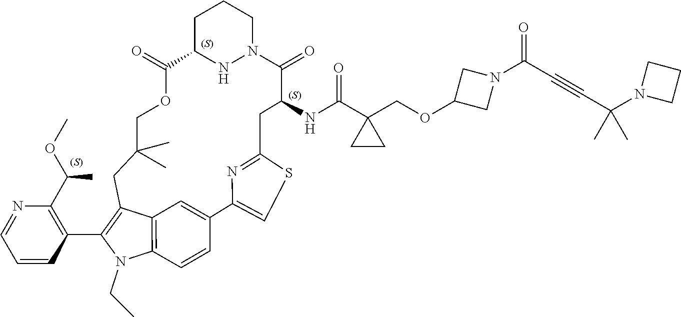 |
| BA363 | 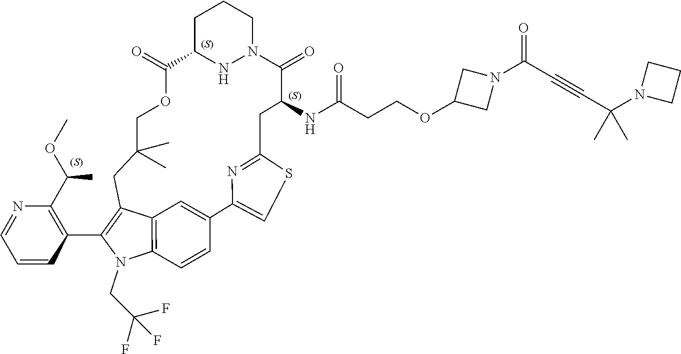 |
| BA364 | 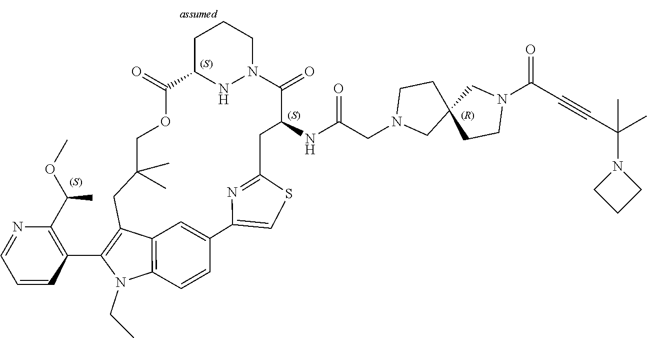 |
| BA365 | 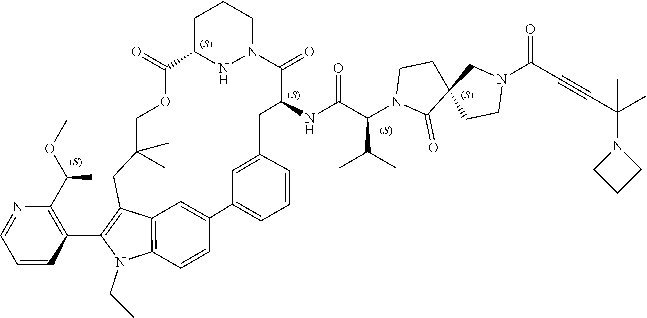 |
| BA366 | 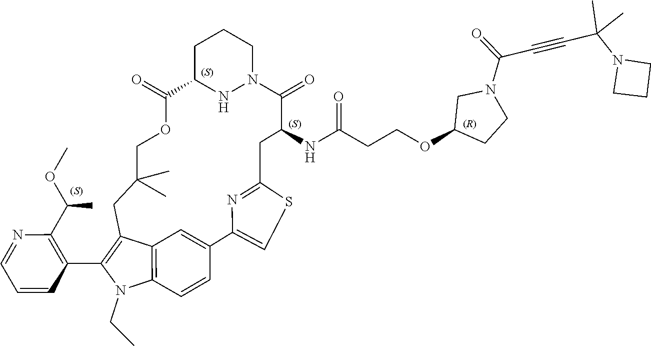 |
| BA367 | 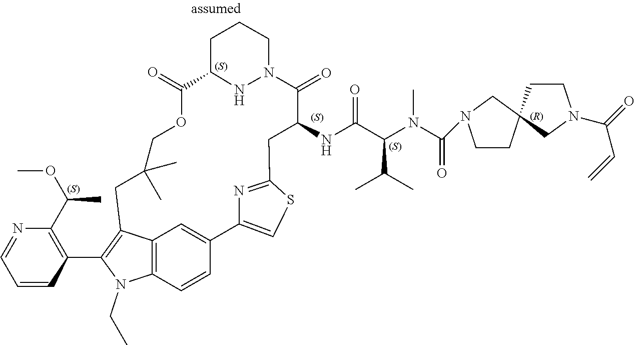 |
| BA368 | 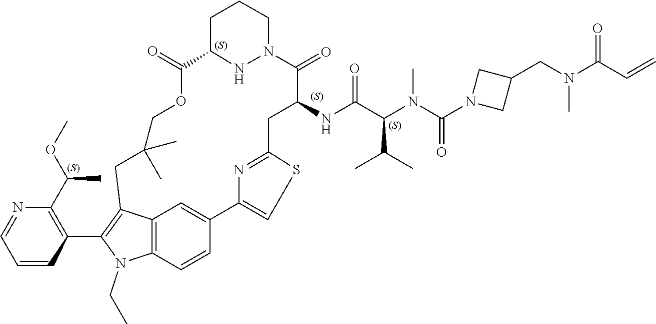 |
| BA369 | 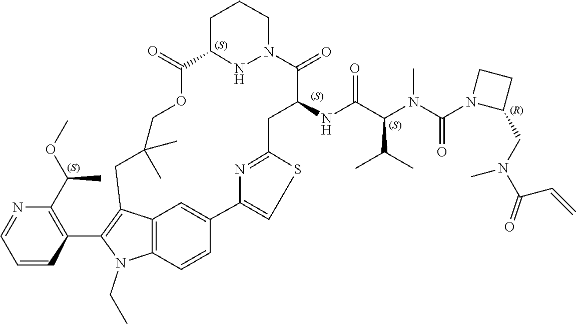 |
| BA370 | 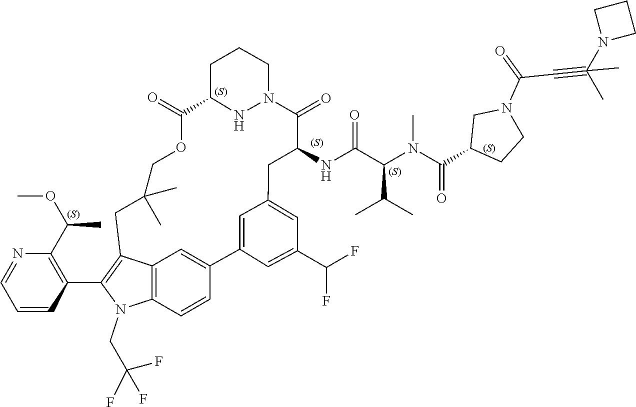 |
| BA371 | 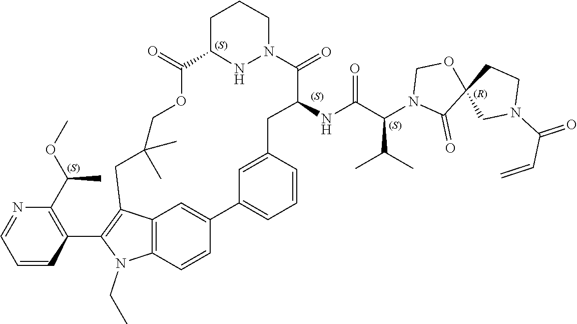 |
| BA372 | 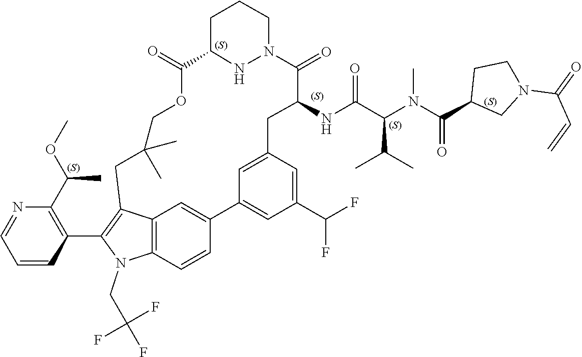 |
| BA373 | 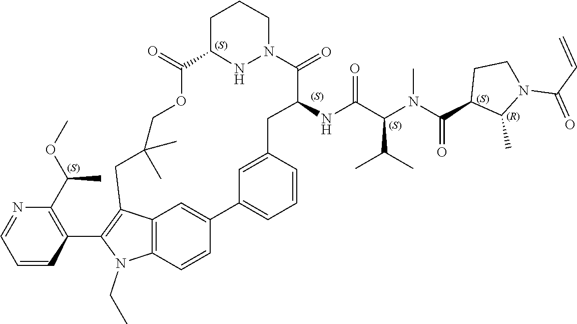 |
| BA374 | 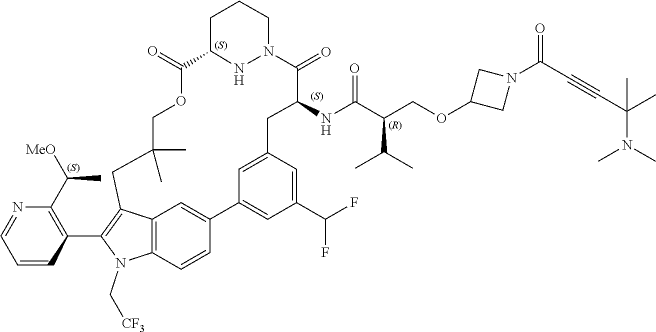 |
| BA375 | 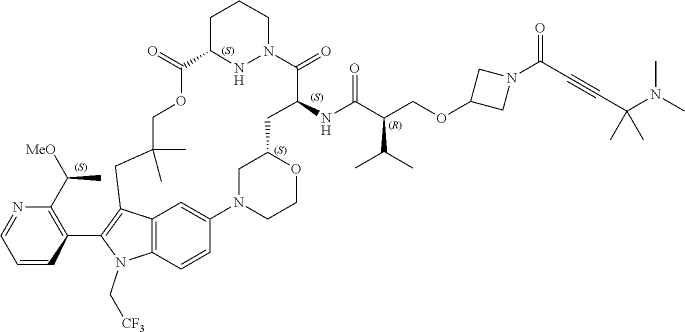 |
| BA376 | 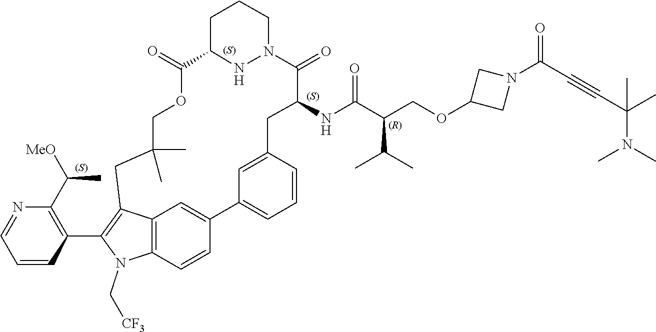 |
| BA377 | 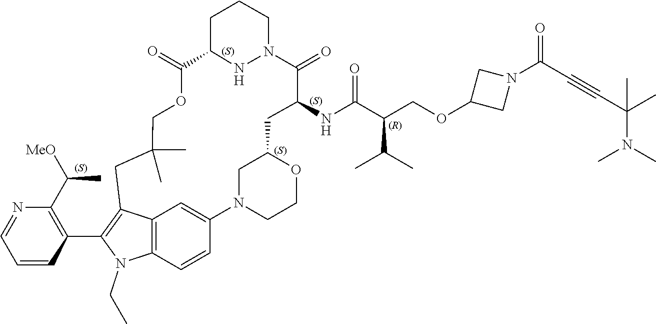 |
| BA378 | 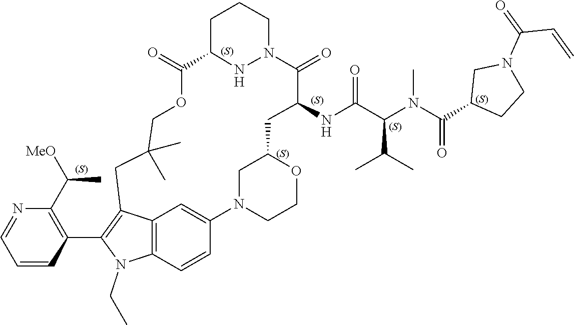 |
| BA379 | 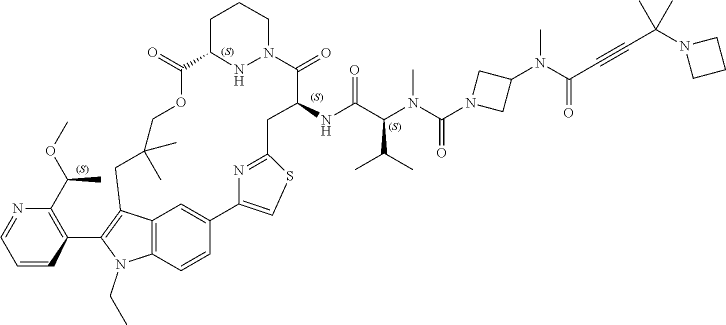 |
| BA380 | 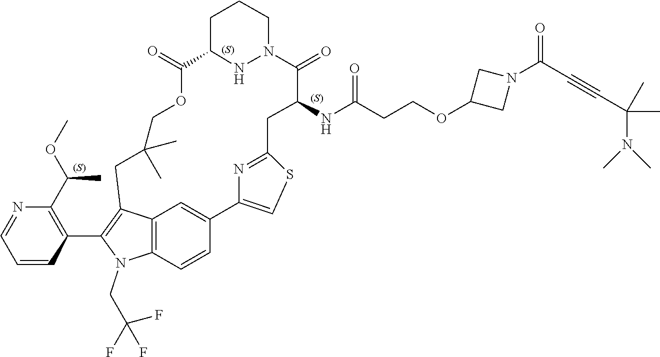 |
| BA381 | 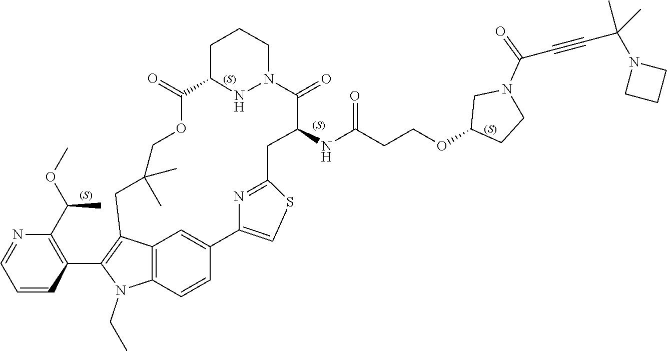 |
| BA382 | 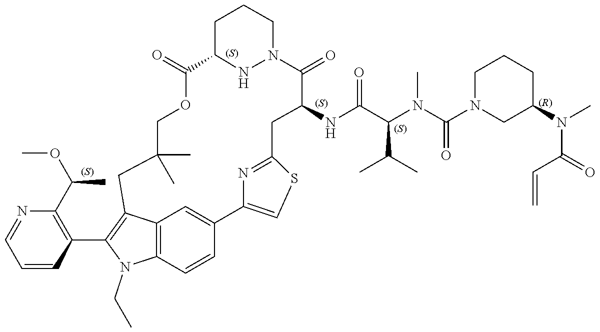 |
| BA383 | 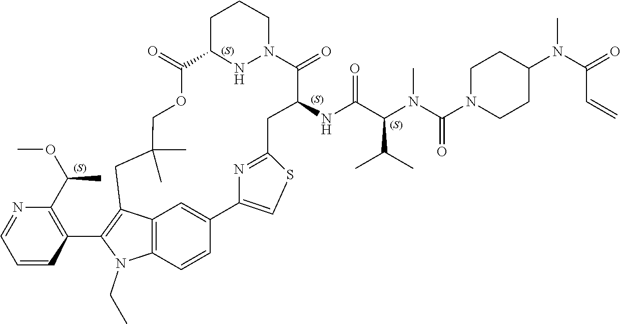 |
| BA384 | 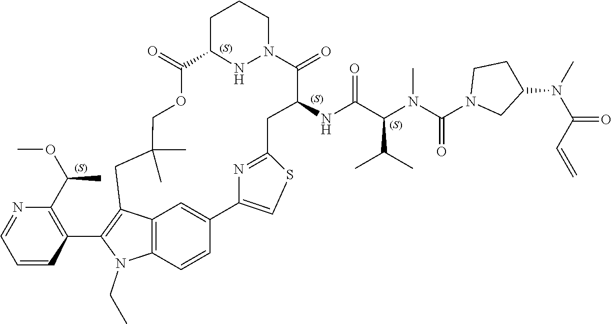 |
| BA385 | 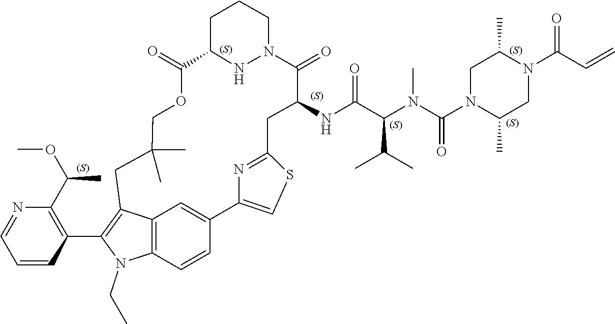 |
| BA386 | 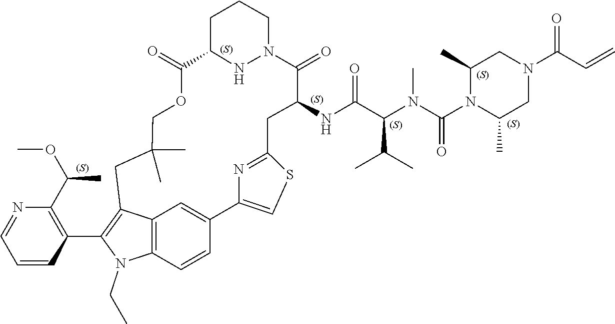 |
| BA387 | 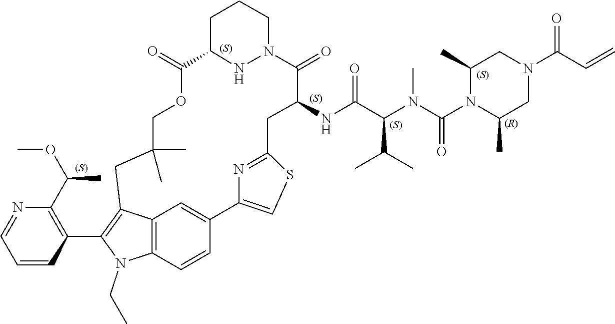 |
| BA388 | 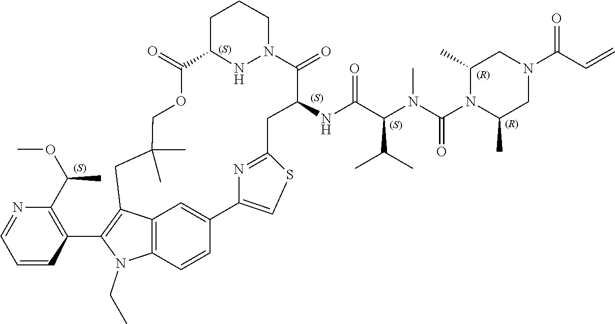 |
| BA389 | 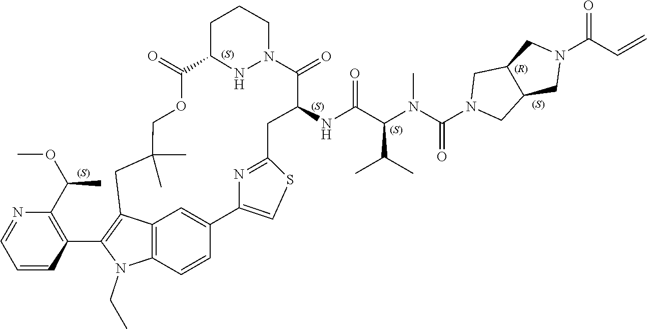 |
| BA390 | 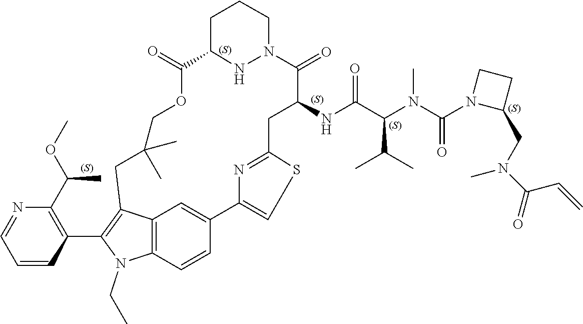 |
| BA391 | 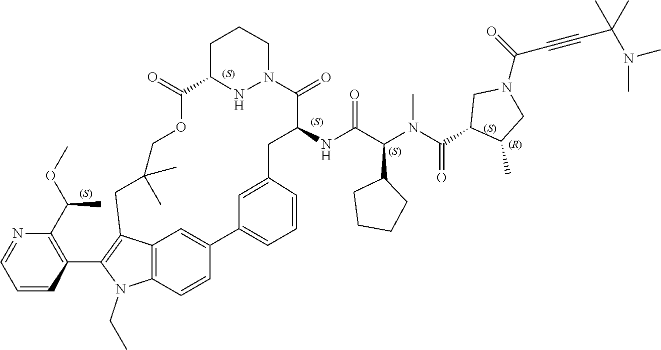 |
| BA392 | 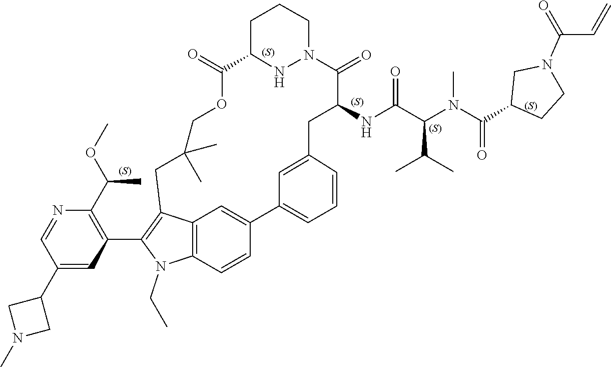 |
| BA393 | 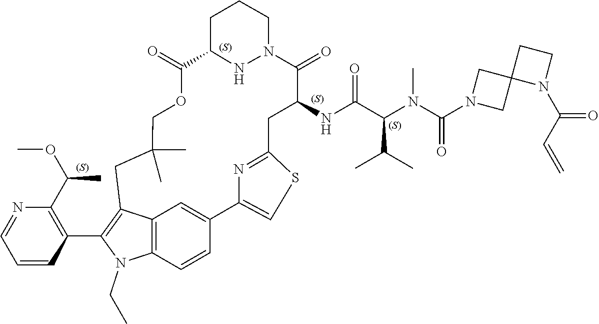 |
| BA394 | 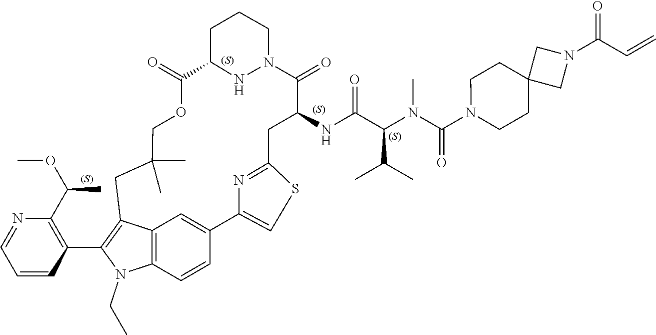 |
| BA395 | 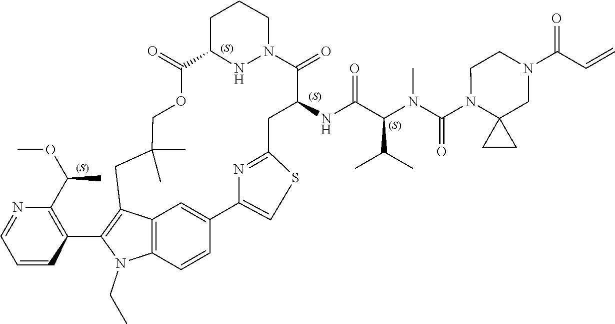 |
| BA396 | 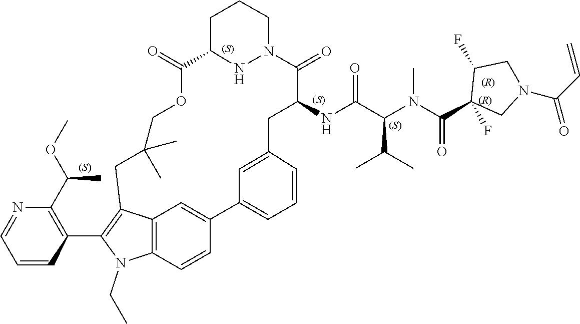 |
| BA397 | 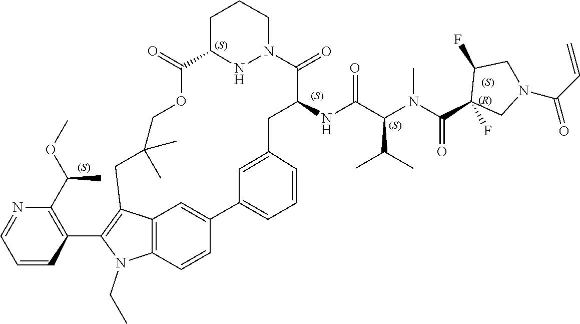 |
| BA398 | 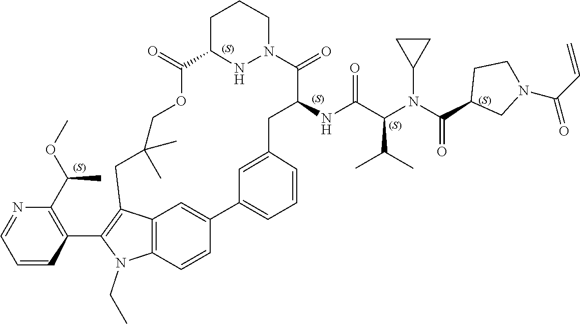 |
| BA399 | 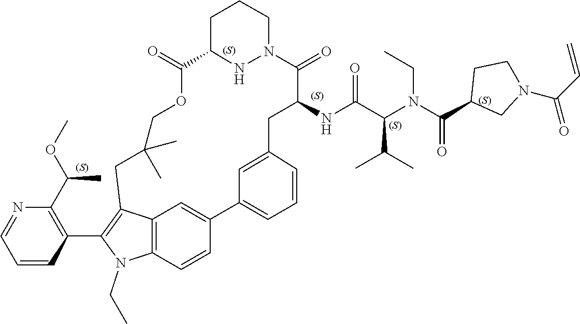 |
| BA400 | 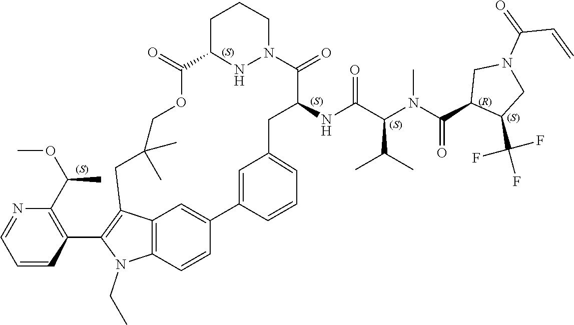 |
| BA401 | 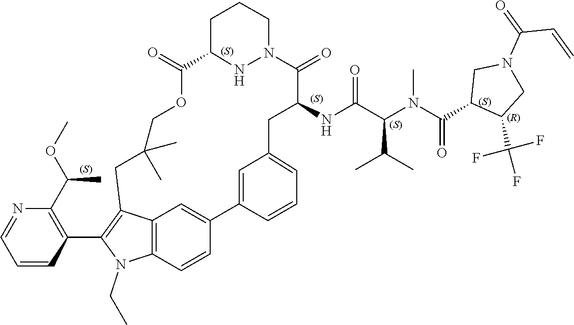 |
| BA402 | 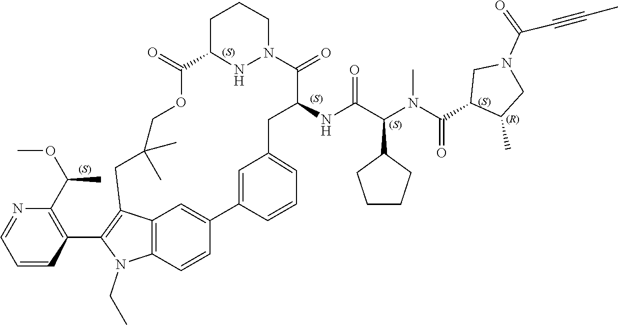 |
| BA403 | 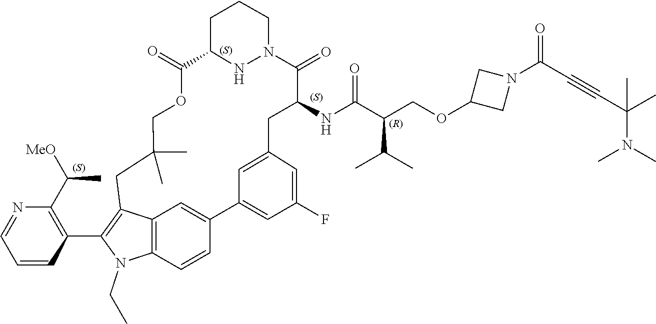 |
| BA404 | 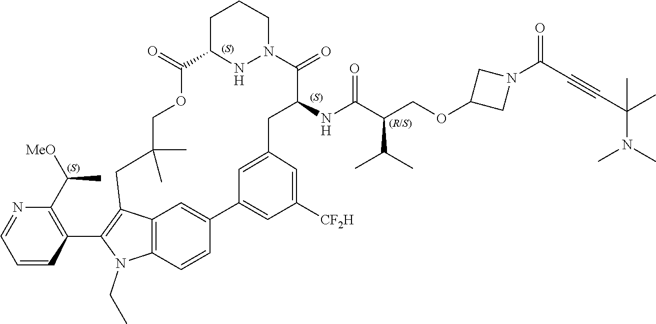 |
| BA405 | 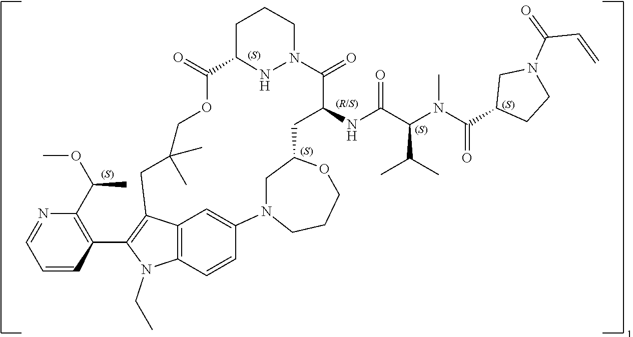 |
| BA406 | 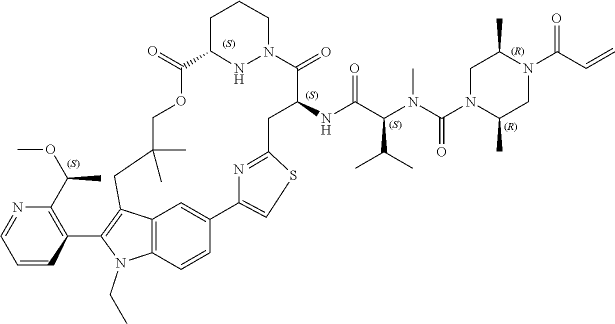 |
| BA407 | 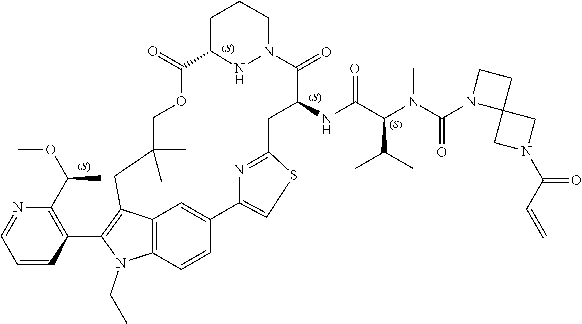 |
| BA408 | 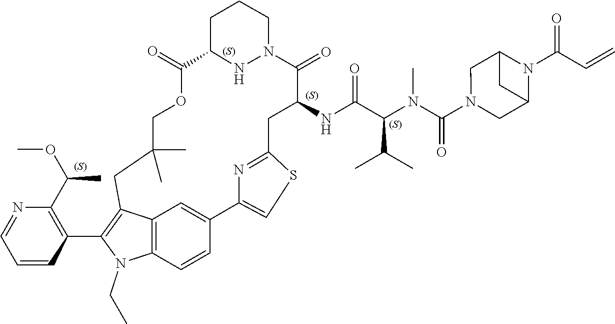 |
| BA409 | 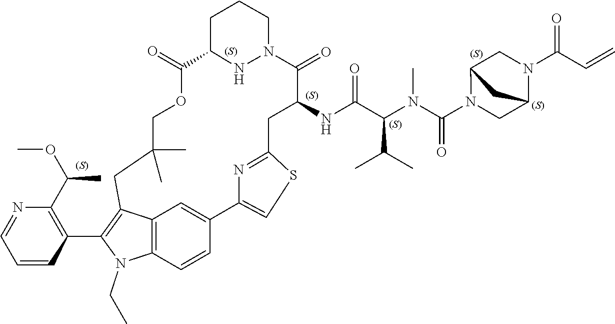 |
| BA410 | 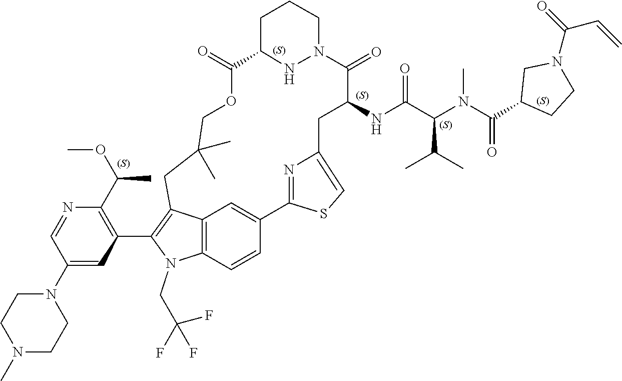 |
| BA411 | 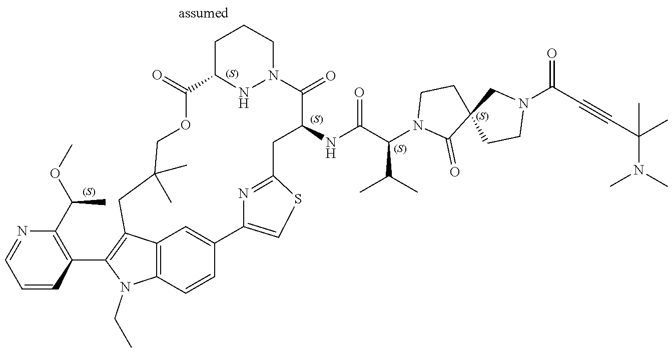 |
| BA412 | 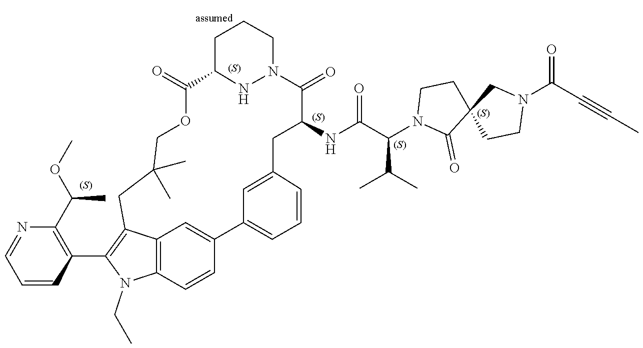 |
| BA413 | 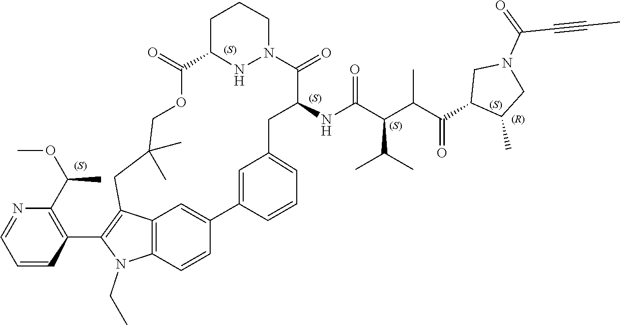 |
| BA414 | 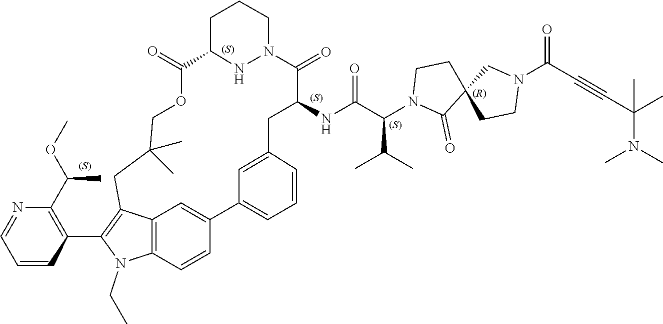 |
| BA415 | 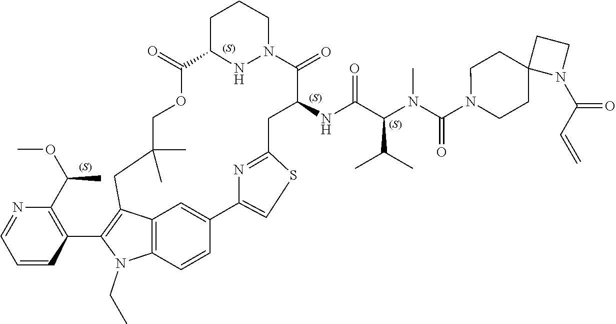 |
| BA416 | 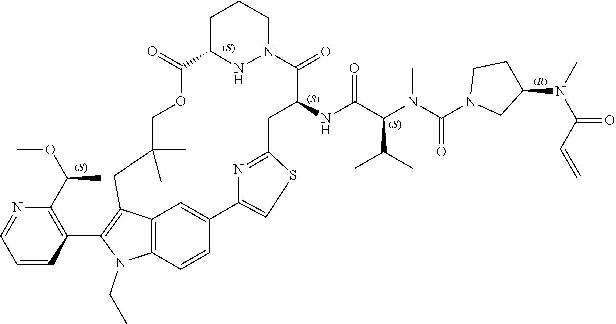 |
| BA417 | 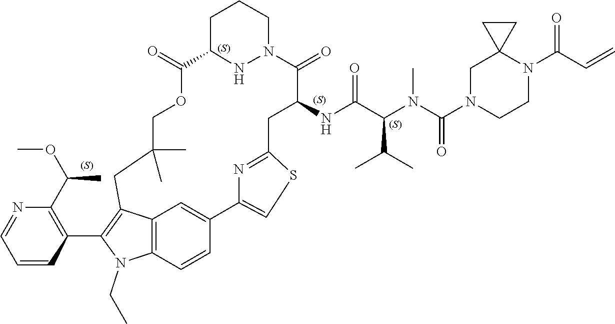 |
| BA418 | 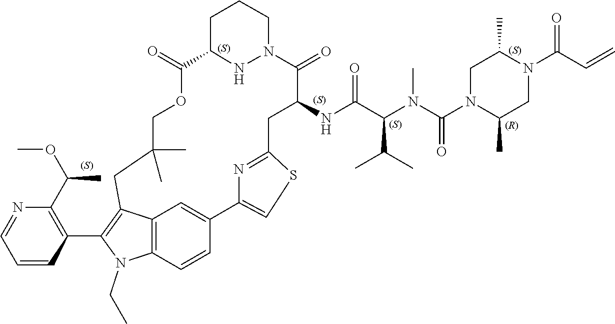 |
| BA419 | 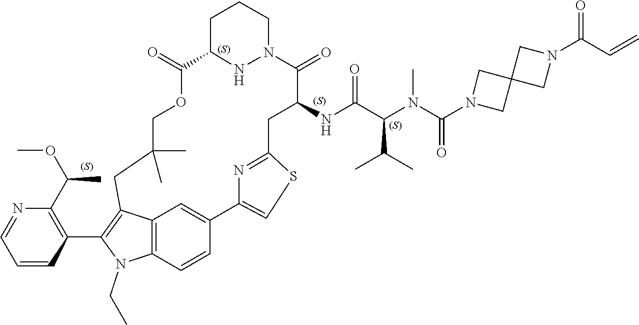 |
| BA420 | 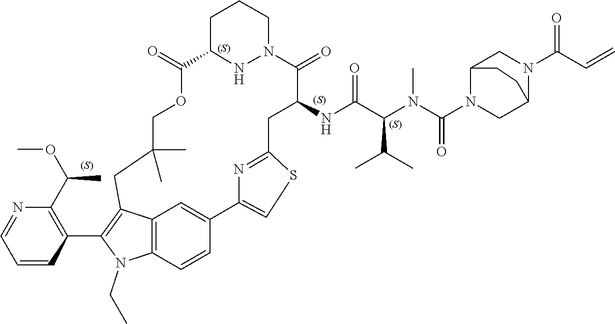 |
| BA421 | 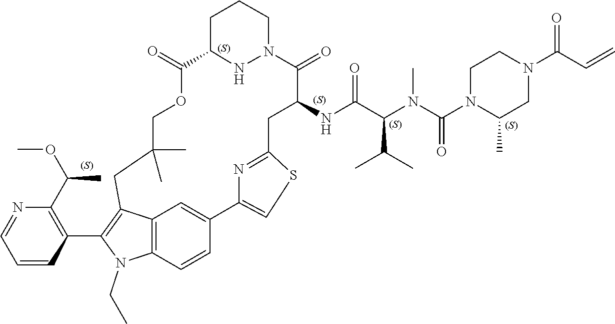 |
| BA422 | 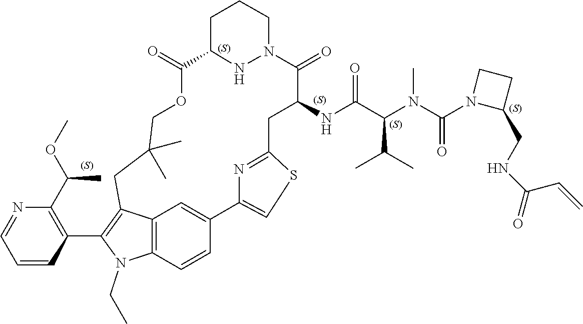 |
| BA423 | 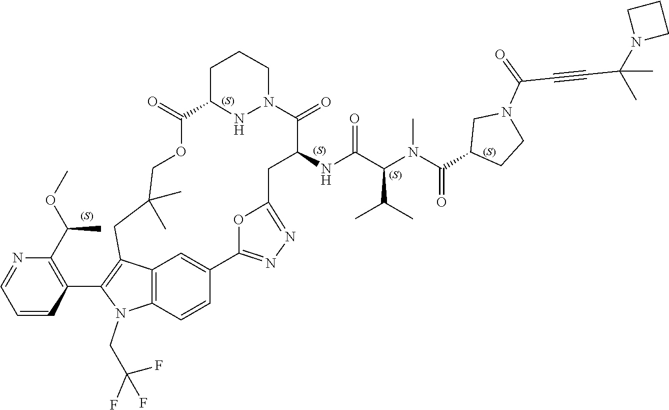 |
| BA424 | 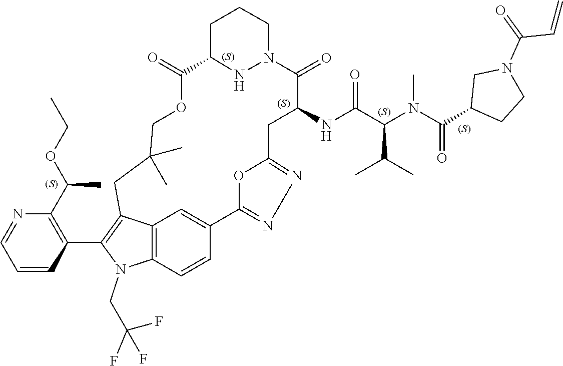 |
| BA425 | 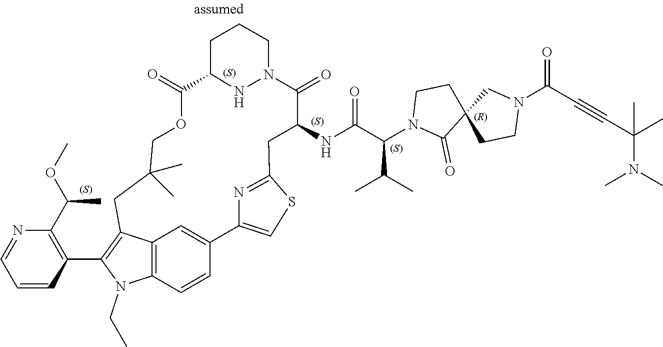 |
| BA426 | 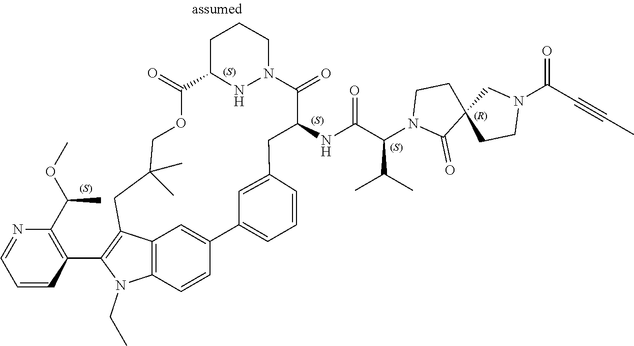 |
| BA427 | 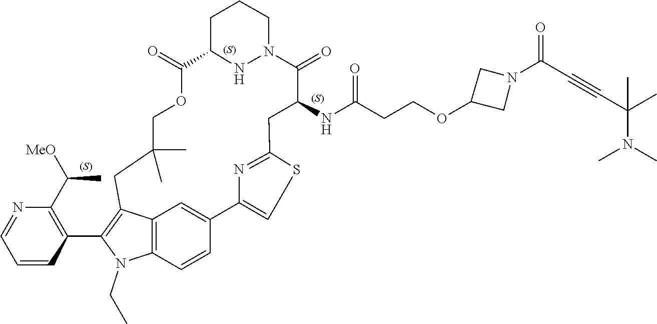 |
| BA428 | 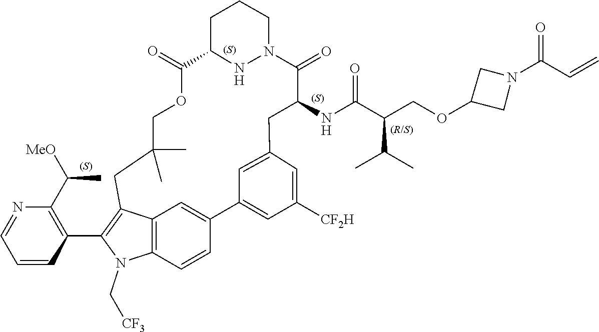 |
| BA429 | 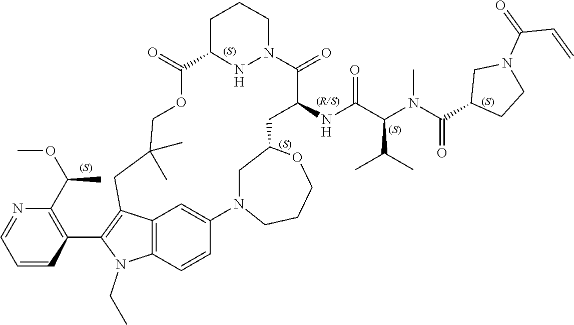 |
| BA430 | 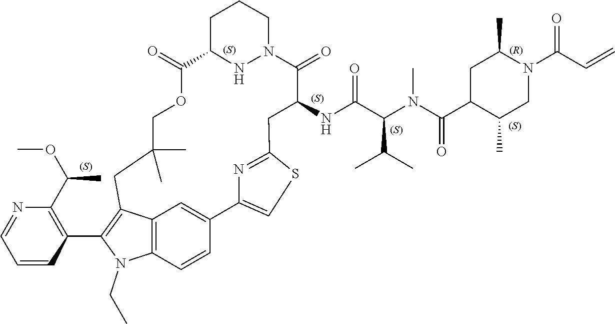 |
| BA431 | 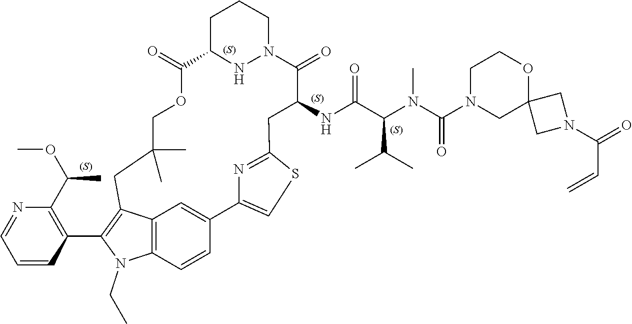 |
| BA432 | 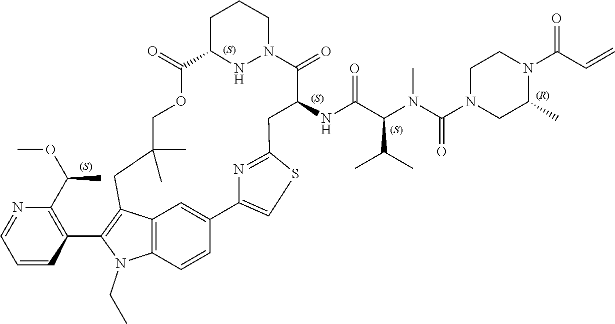 |
| BA433 | 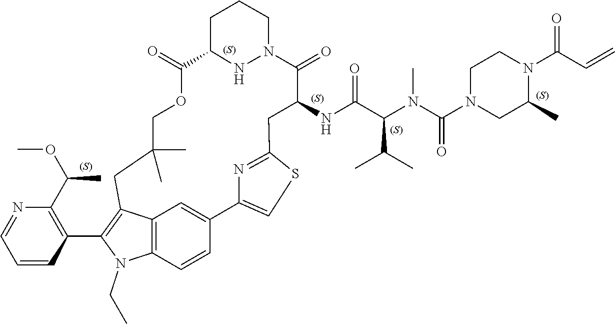 |
| BA334 | 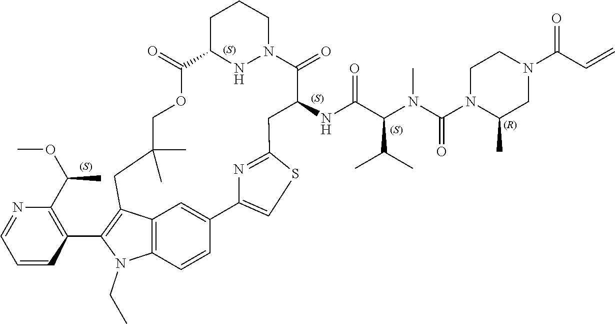 |
| BA435 | 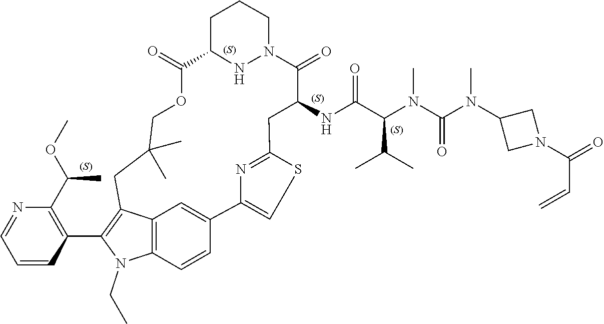 |
| BA436 | 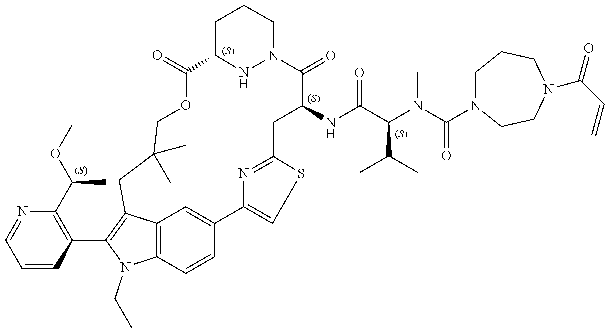 |
| BA437 | 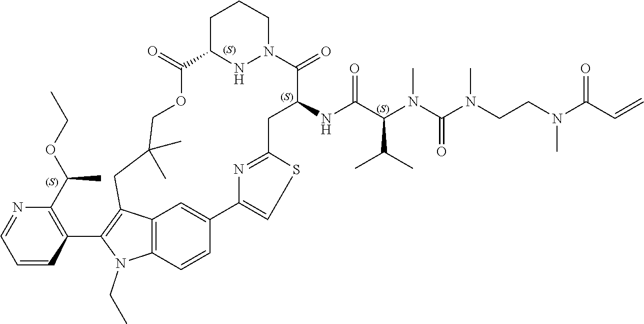 |
| BA438 | 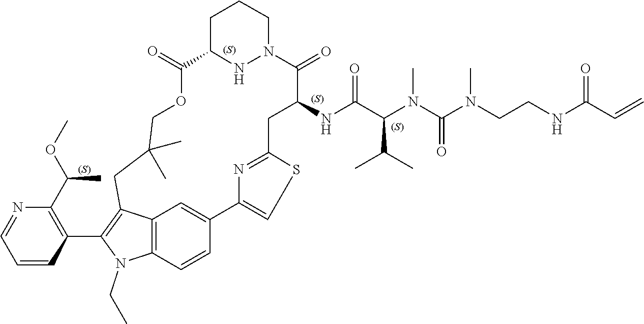 |
| BA439 | 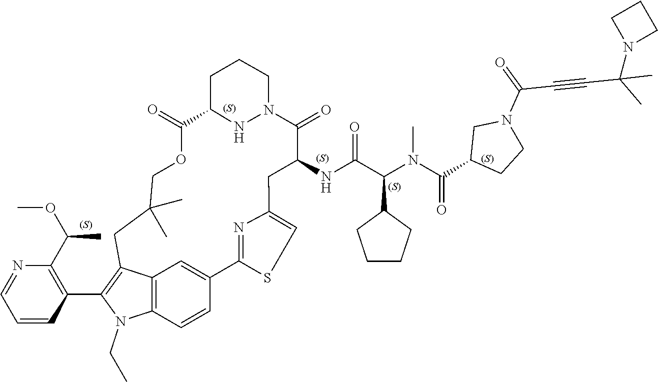 |
| BA440 | 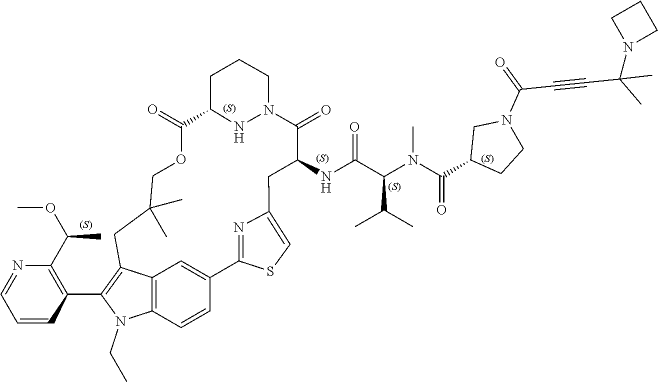 |
| BA441 | 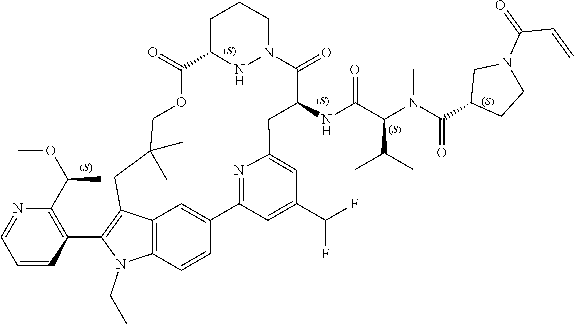 |
| BA442 | 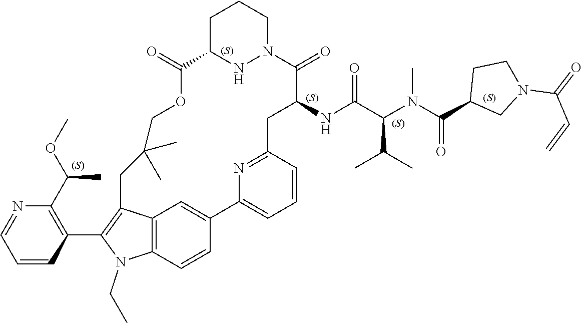 |
| BA443 | 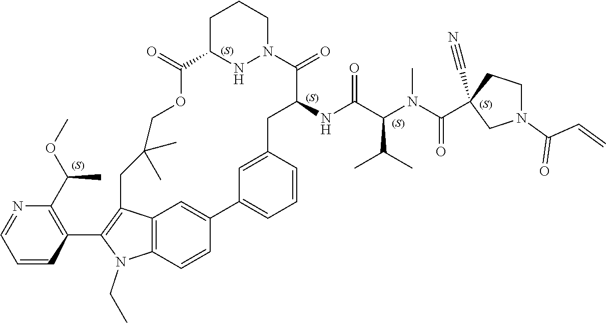 |
| BA444 | 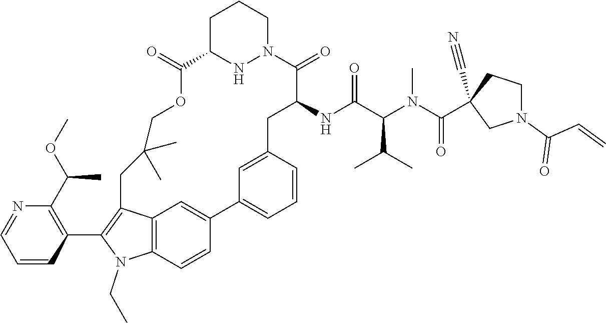 |
| BA445 | 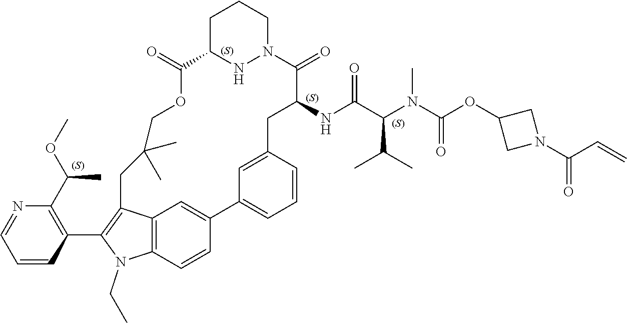 |
| BA446 | 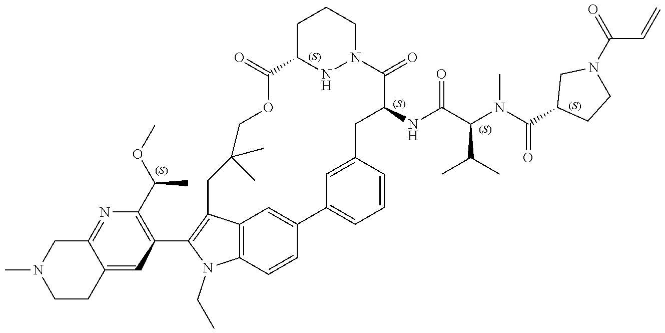 |
| BA447 | 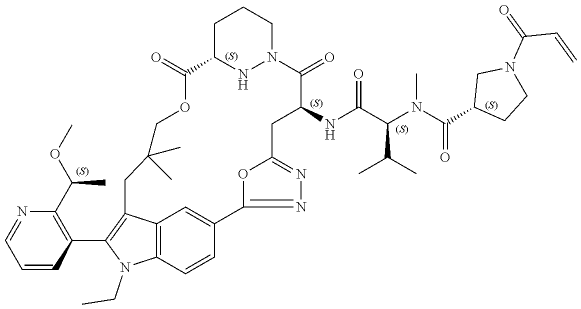 |
| BA448 | 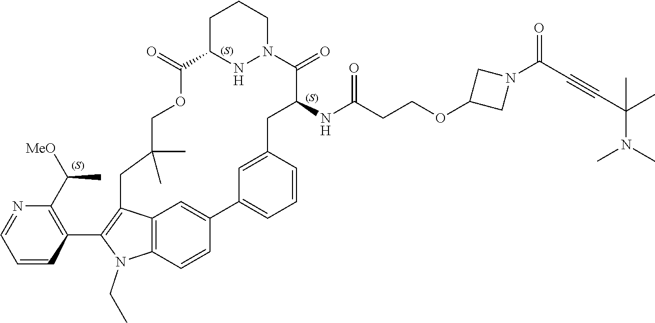 |
| BA449 | 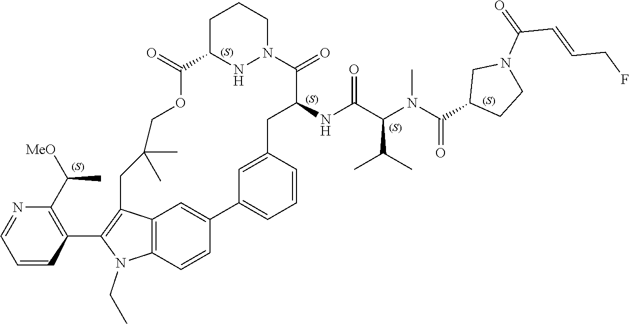 |
| BA450 | 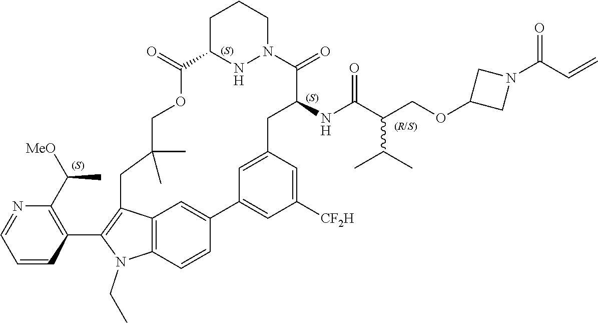 |
| BA451 | 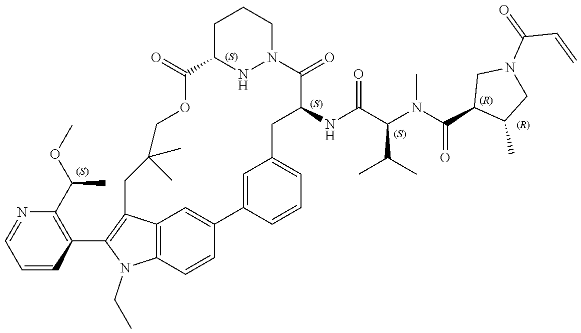 |
| BA452 | 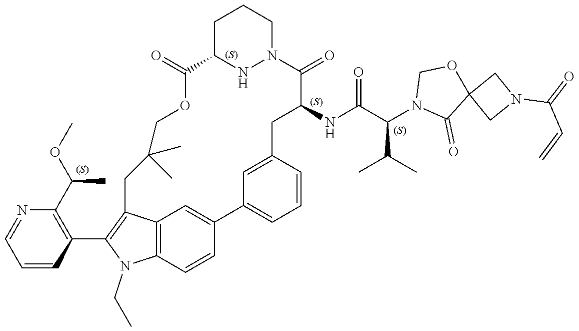 |
| BA453 | 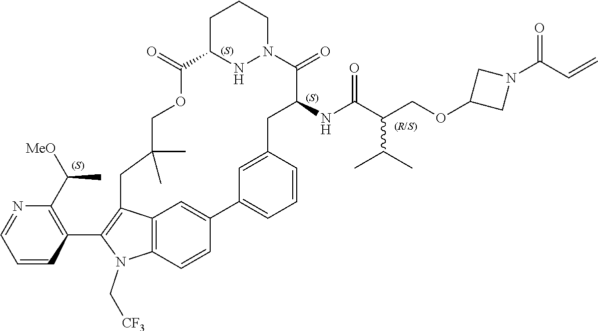 |
| BA454 | 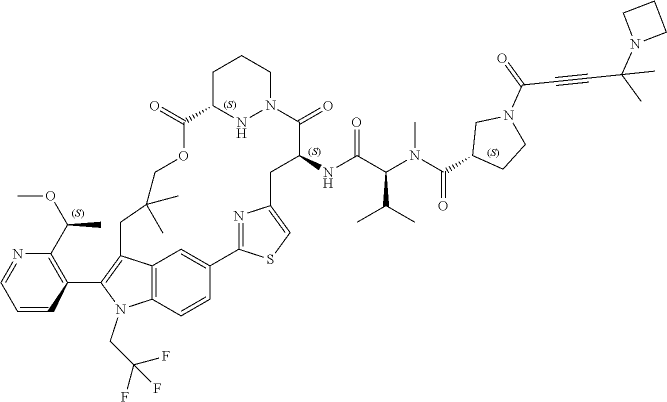 |
| BA455 | 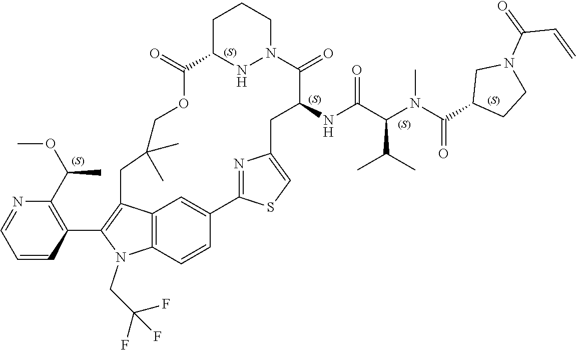 |
| BA456 | 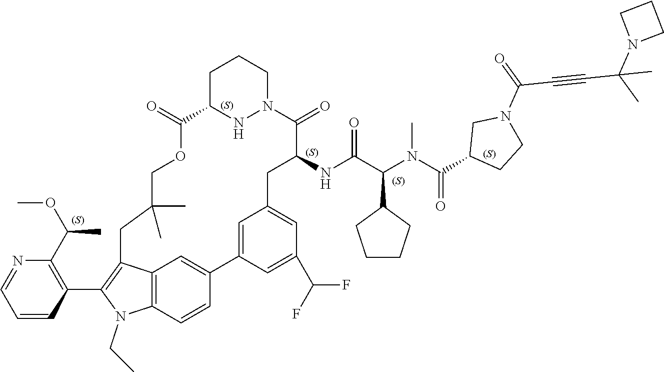 |
| BA457 | 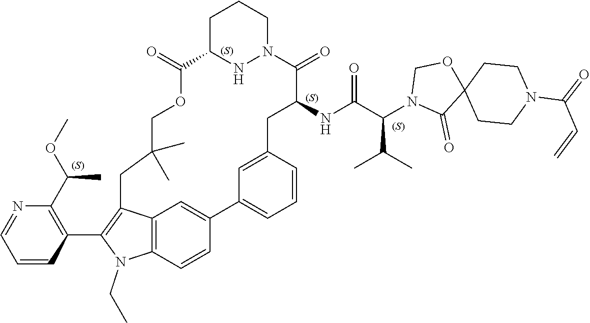 |
| BA458 | 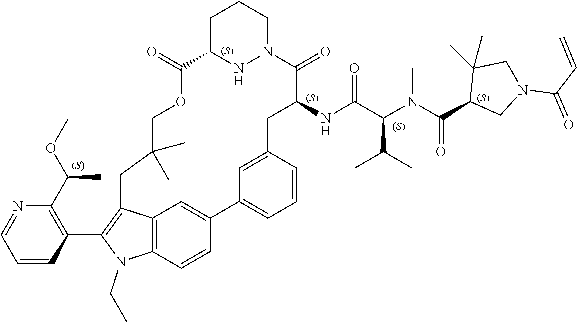 |
| BA459 | 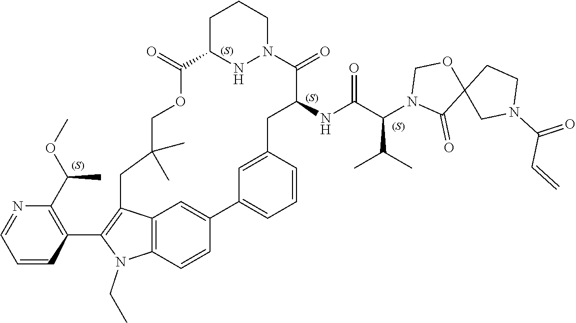 |
| BA460 | 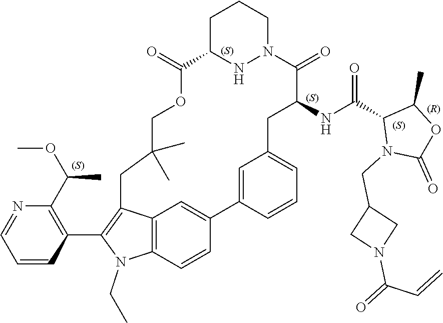 |
| BA461 | 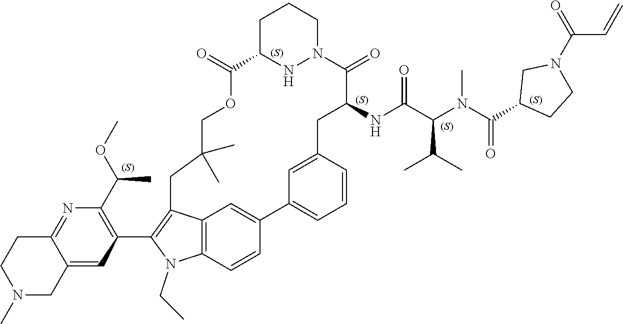 |
| BA462 | 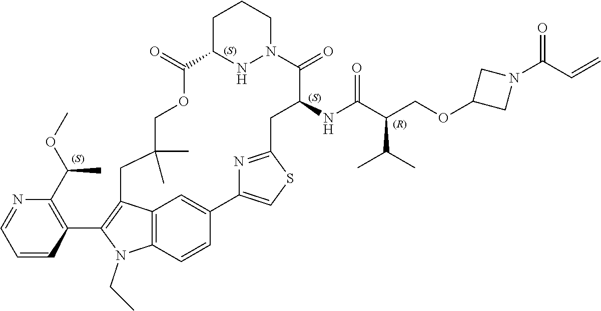 |
| BA463 | 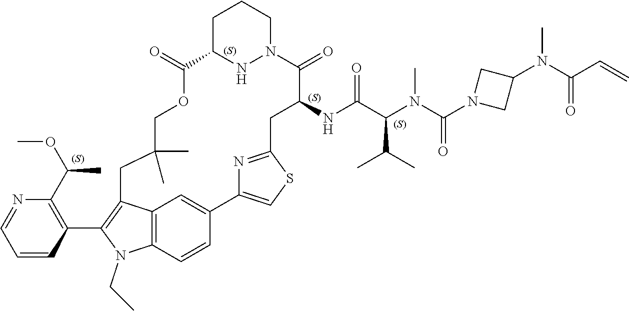 |
| BA464 | 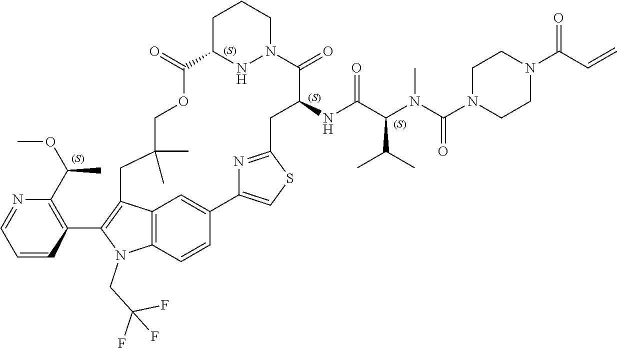 |
| BA465 | 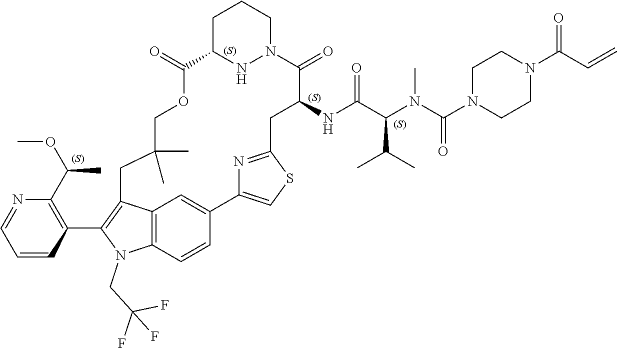 |
| BA466 | 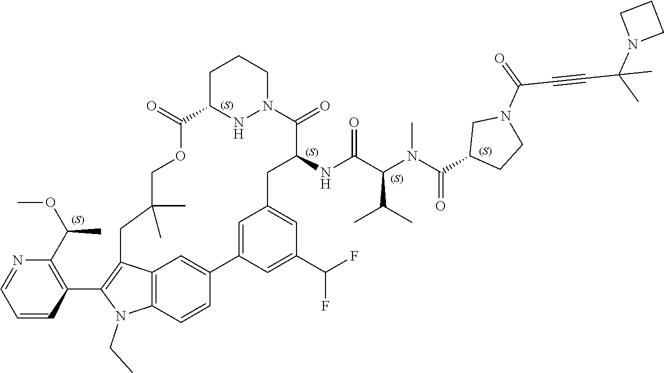 |
| BA467 | 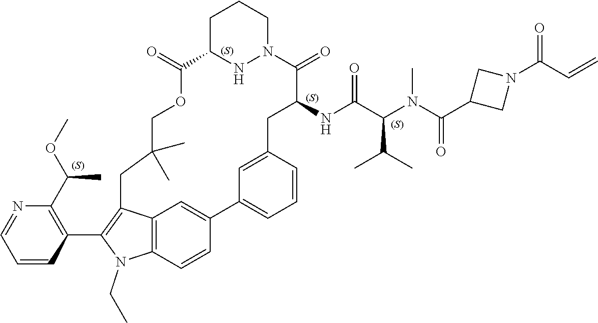 |
| BA468 | 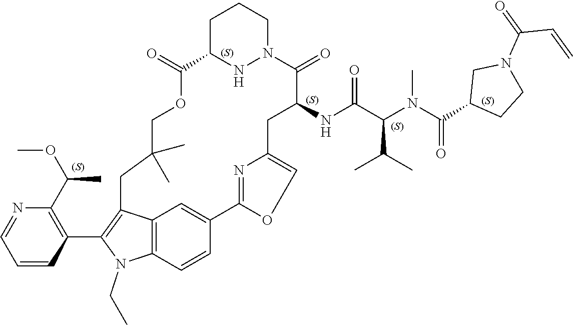 |
| BA469 | 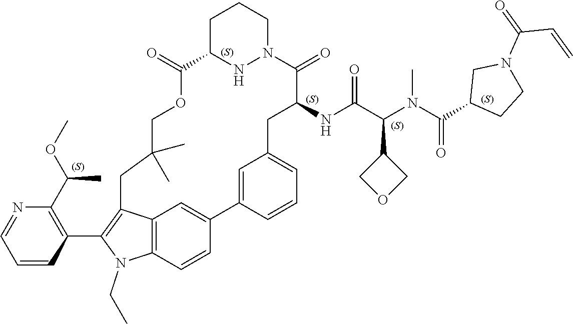 |
| BA470 | 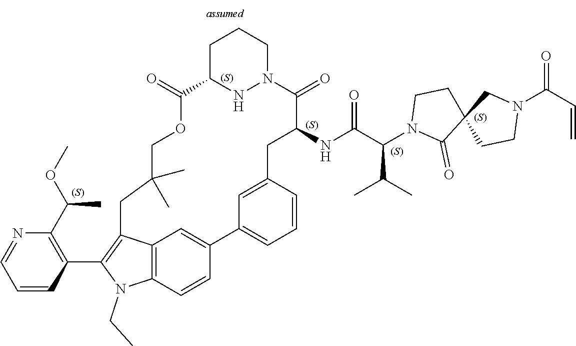 |
| BA471 | 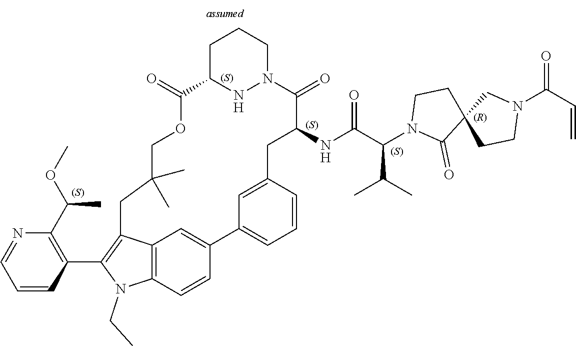 |
| BA472 | 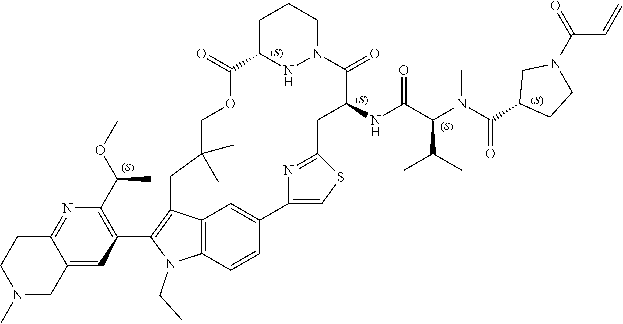 |
| BA473 | 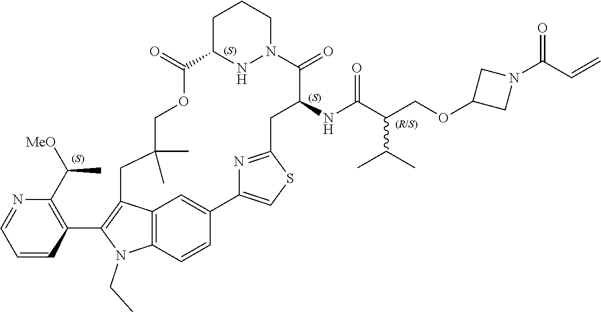 |
| BA474 | 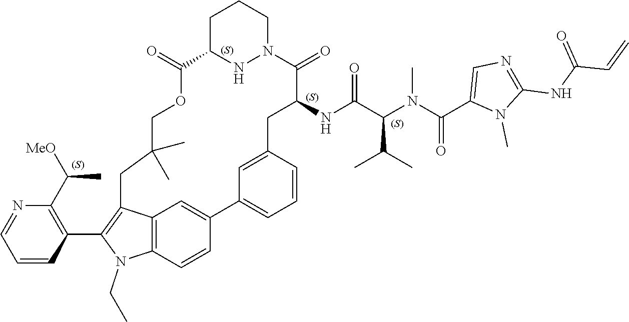 |
| BA475 | 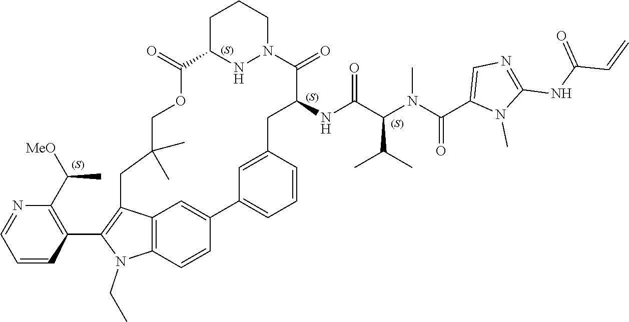 |
| BA476 | 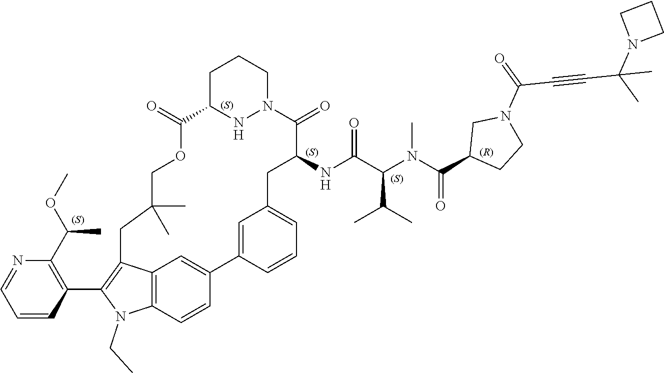 |
| BA477 | 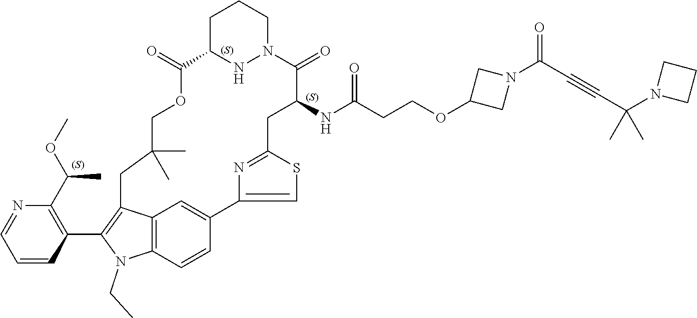 |
| BA478 | 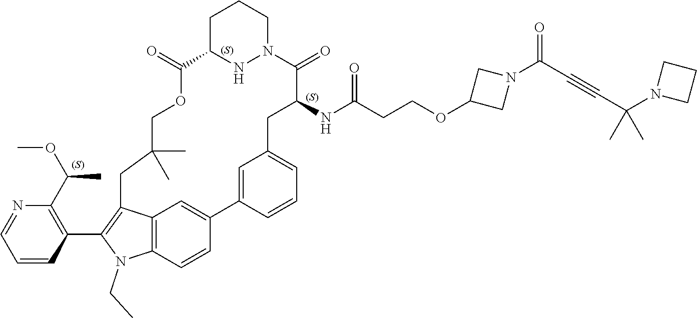 |
| BA479 | 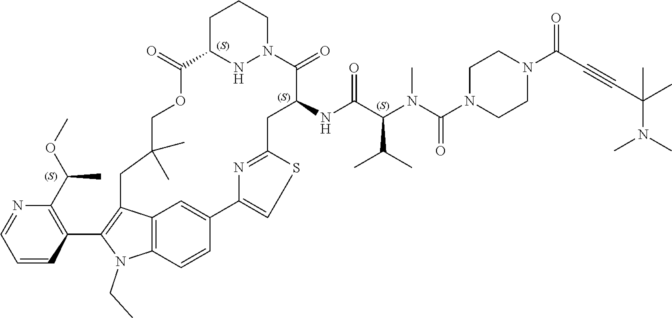 |
| BA480 | 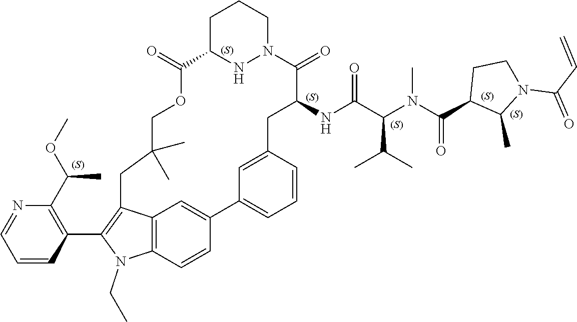 |
| BA481 | 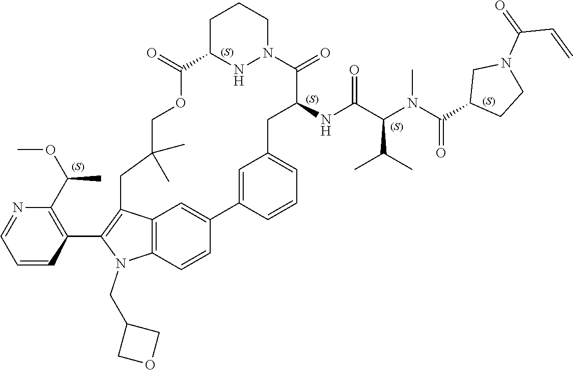 |
| BA482 | 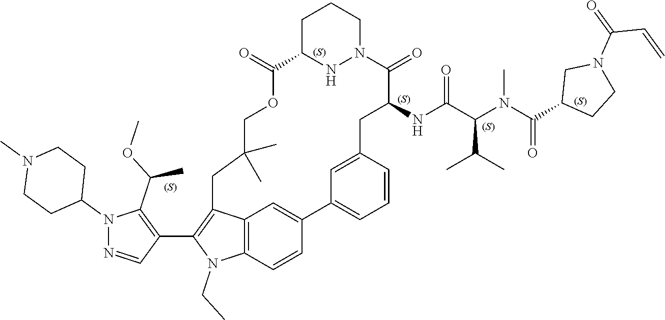 |
| BA483 | 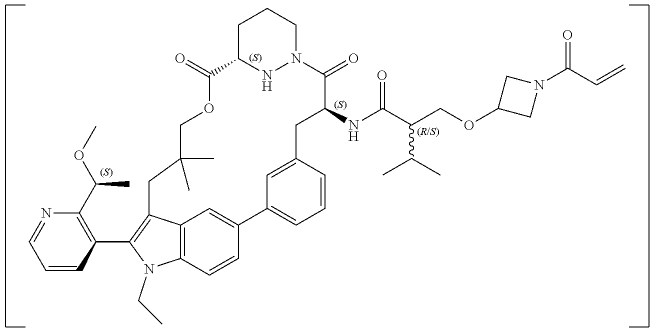 |
| BA484 | 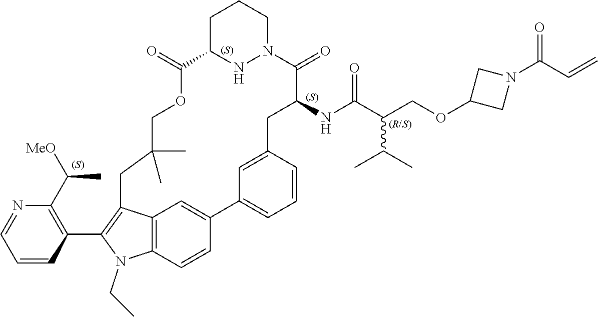 |
| BA485 | 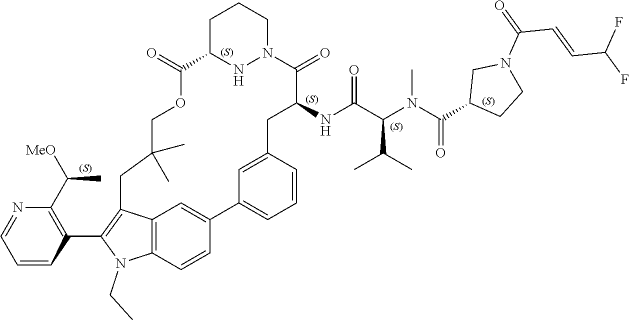 |
| BA486 | 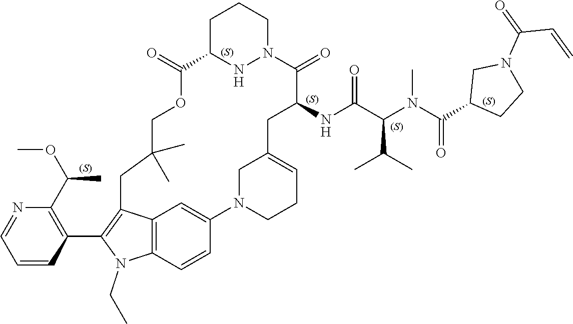 |
| BA487 | 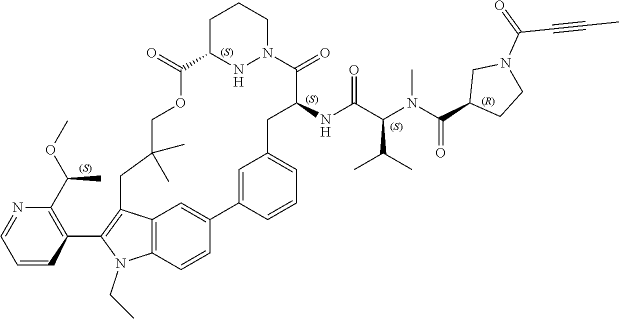 |
| BA488 | 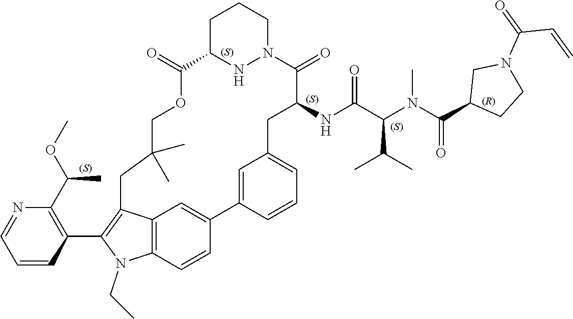 |
| BA489 | 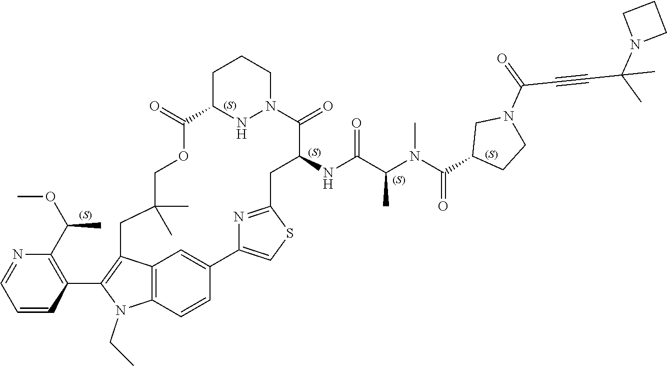 |
| BA490 | 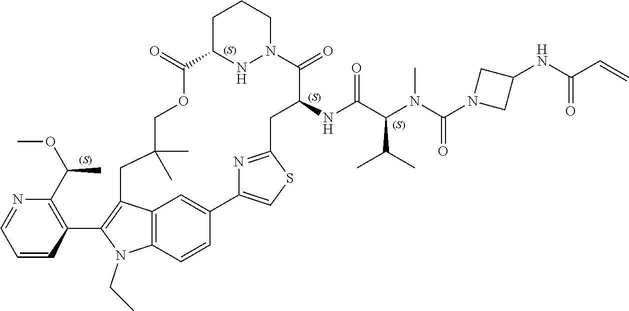 |
| BA491 | 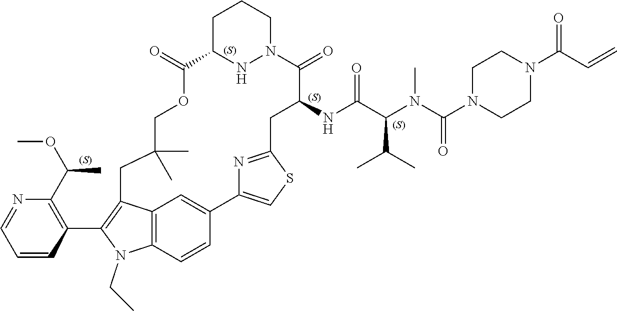 |
| BA492 | 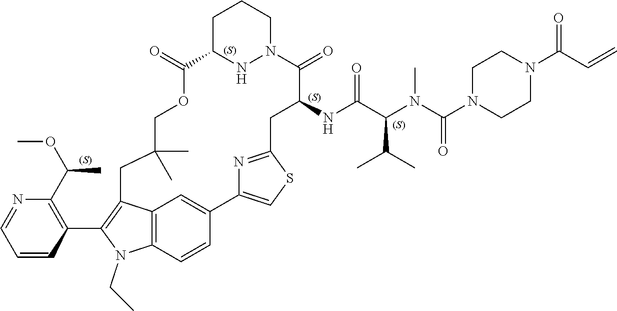 |
| BA493 | 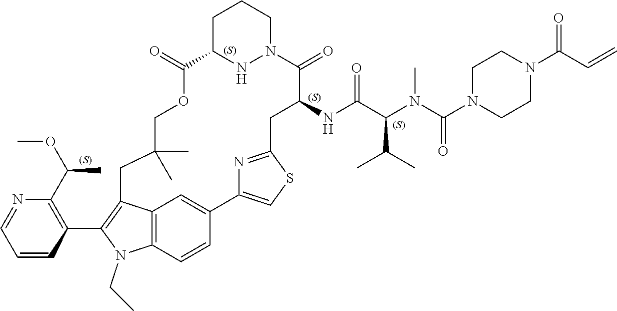 |
| BA494 | 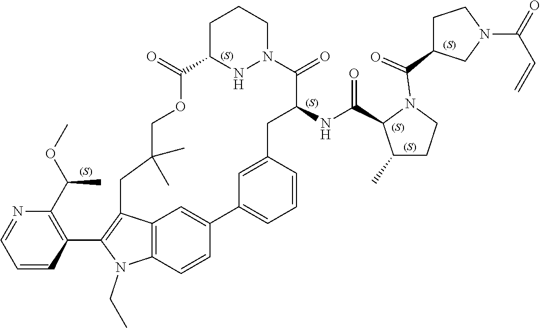 |
| BA495 | 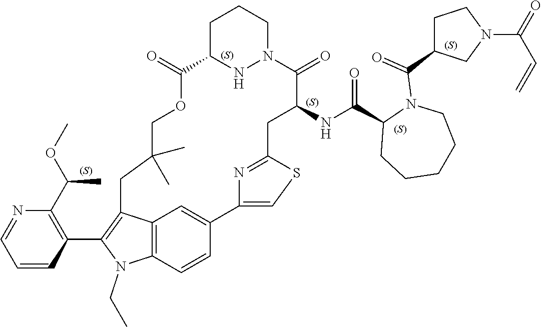 |
| BA496 | 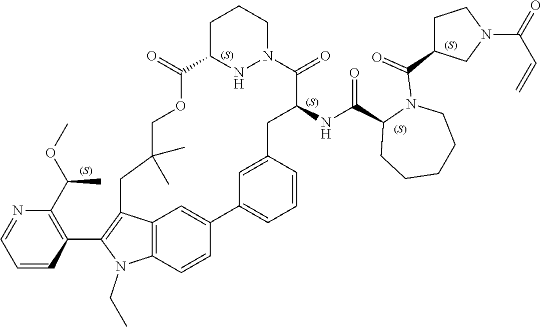 |
| BA497 | 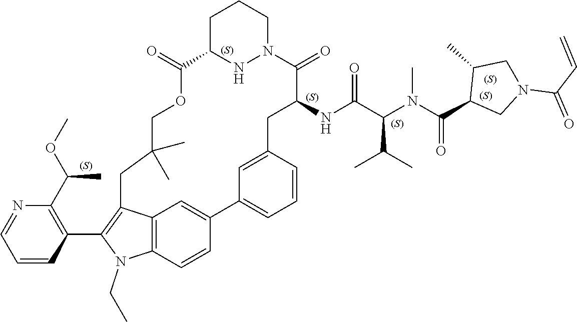 |
| BA498 | 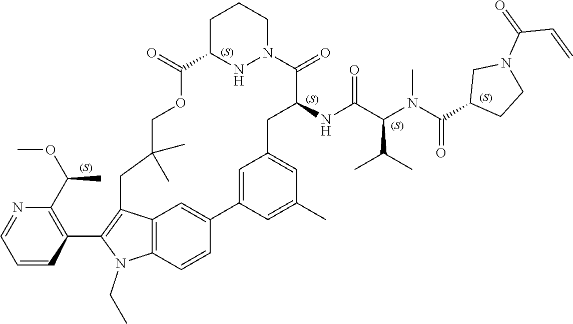 |
| BA499 | 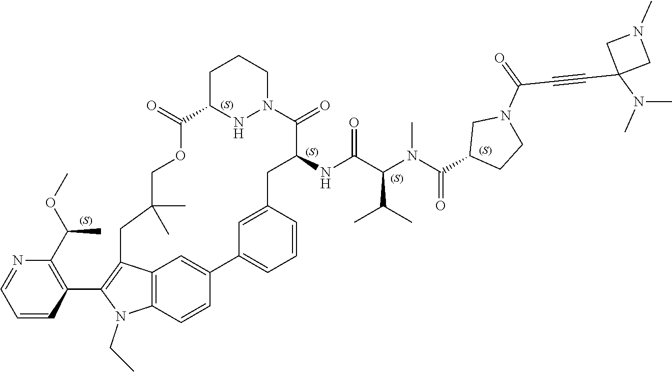 |
| BA500 | 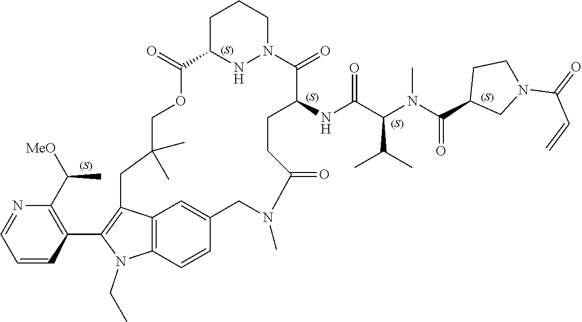 |
| BA501 | 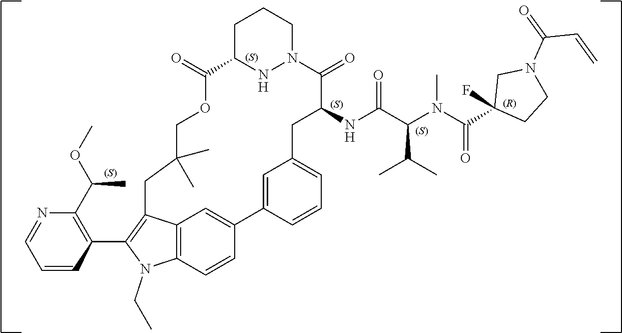 |
| BA502 | 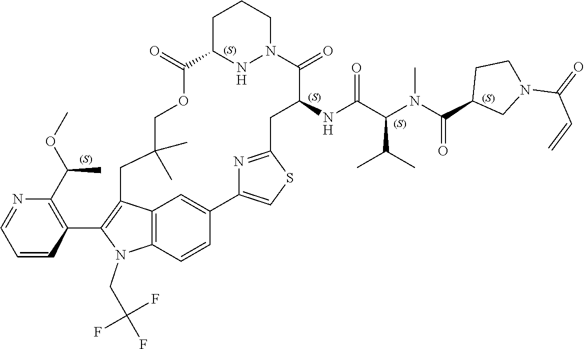 |
| BA503 | 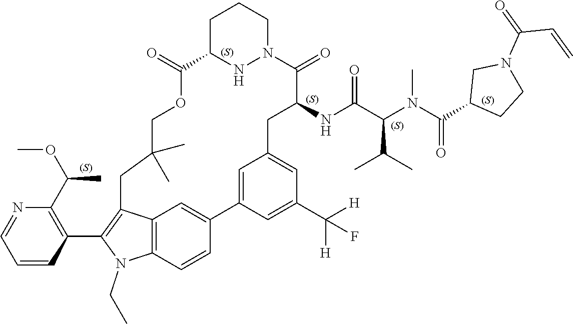 |
| BA504 | 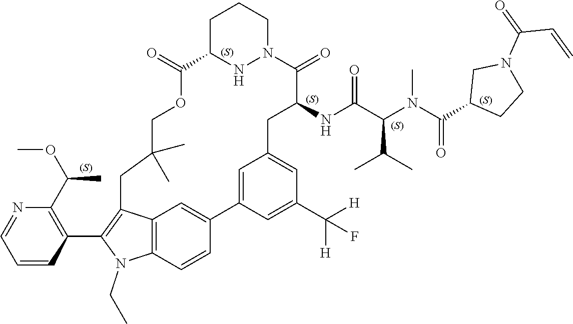 |
| BA505 | 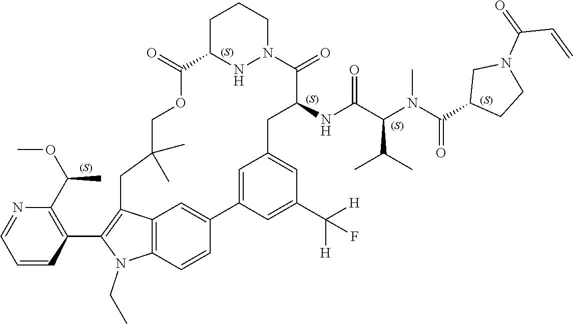 |
| BA506 | 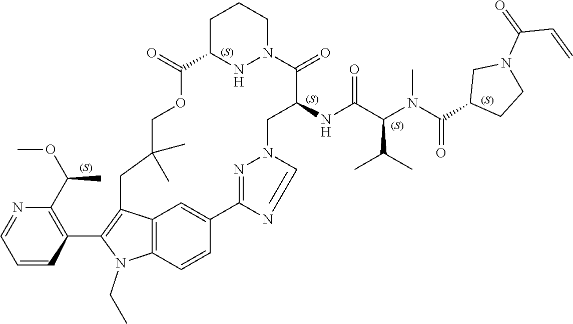 |
| BA507 | 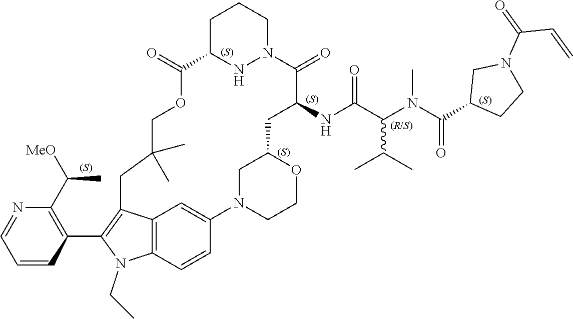 |
| BA508 | 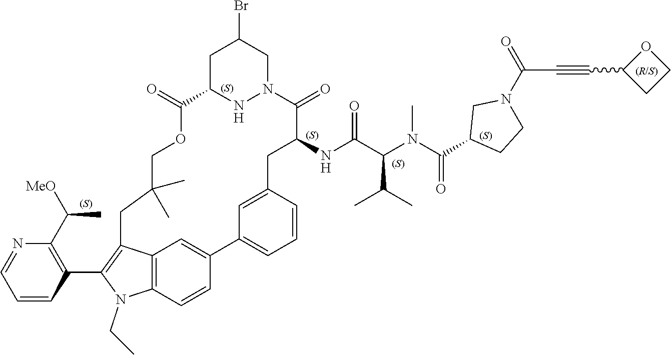 |
| BA509 | 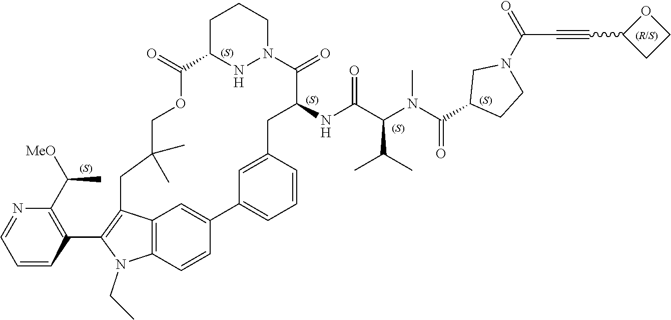 |
| BA510 | 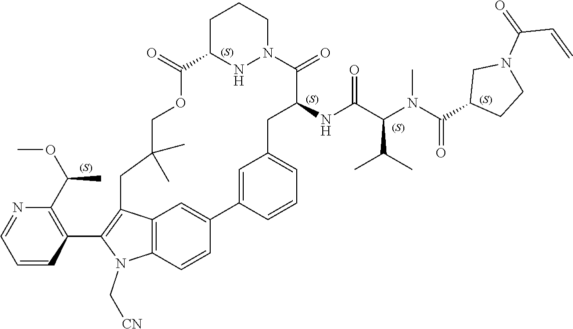 |
| BA511 | 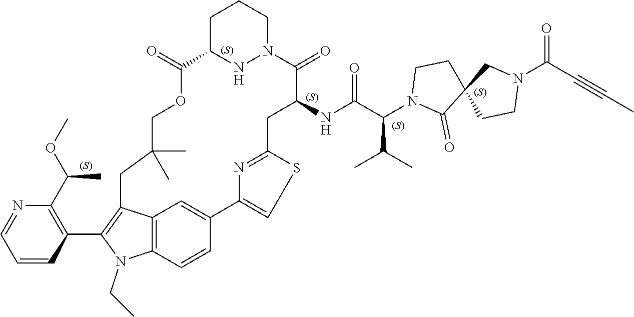 |
| BA512 | 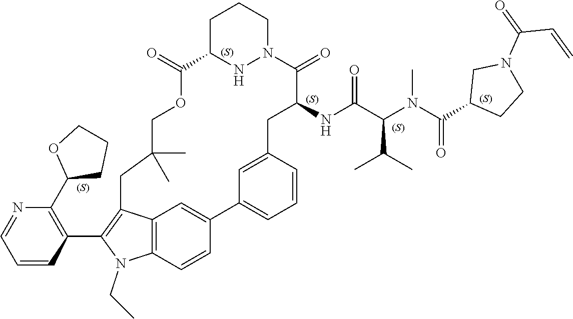 |
| BA513 | 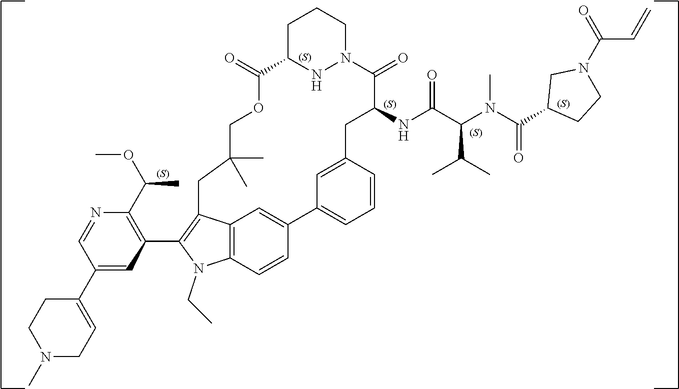 |
| BA514 | 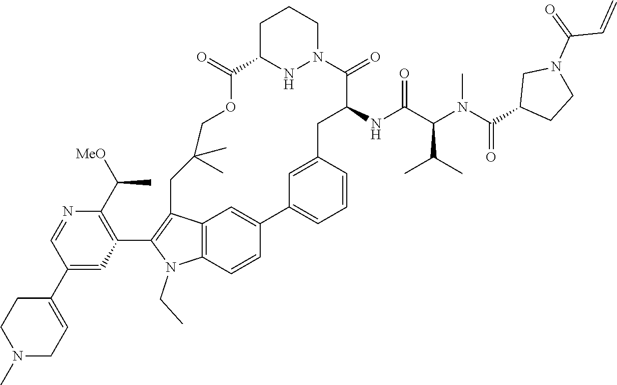 |
| BA515 | 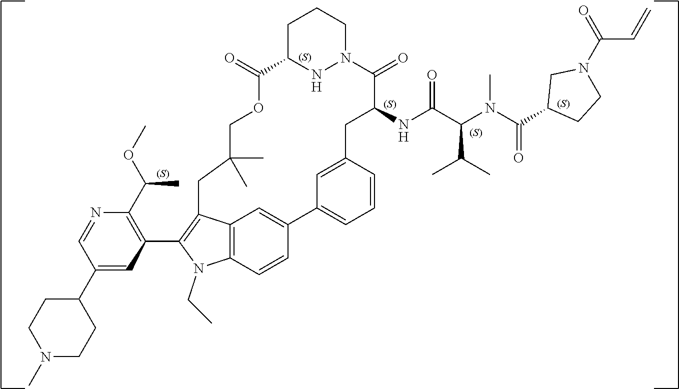 |
| BA516 | 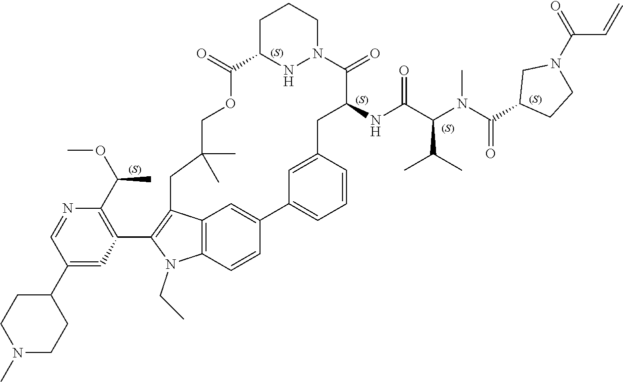 |
| BA517 | 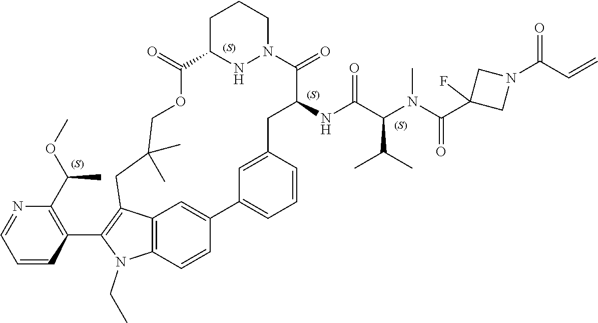 |
| BA518 | 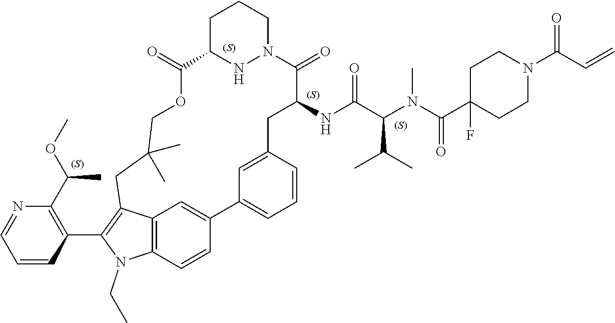 |
| BA519 | 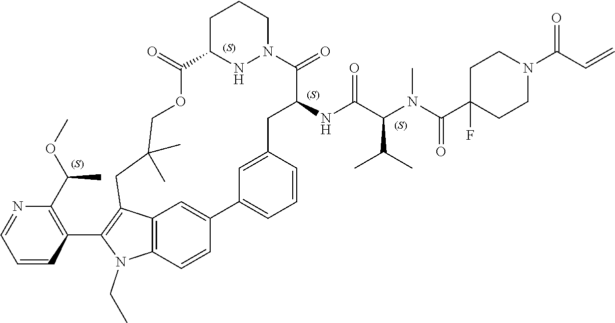 |
| BA520 | 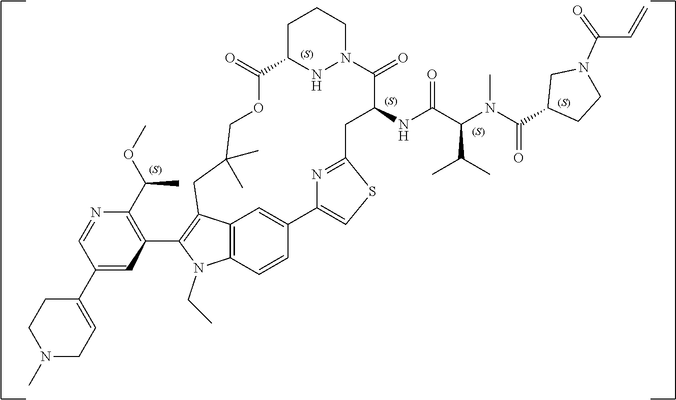 |
| BA521 | 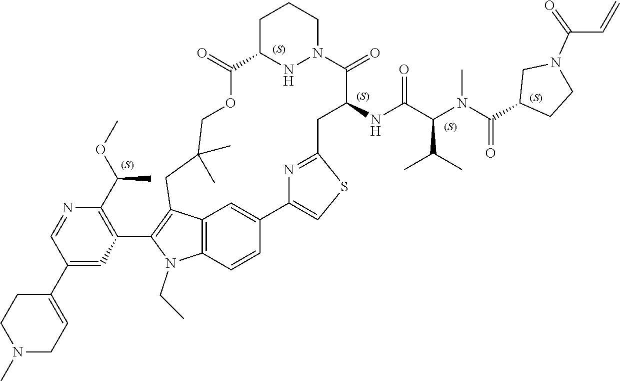 |
| BA522 | 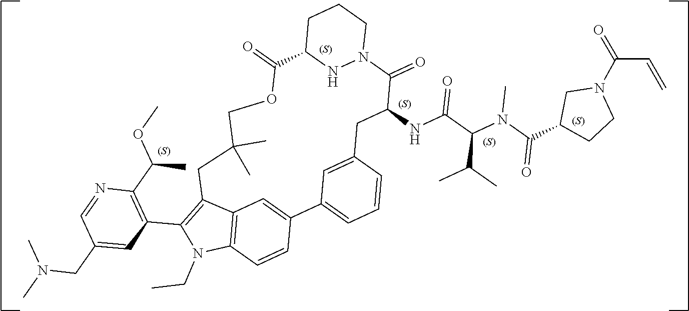 |
| BA523 | 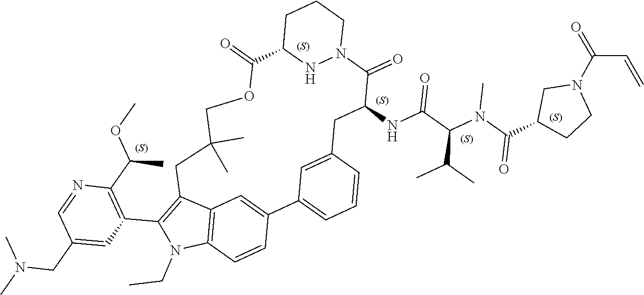 |
| BA524 | 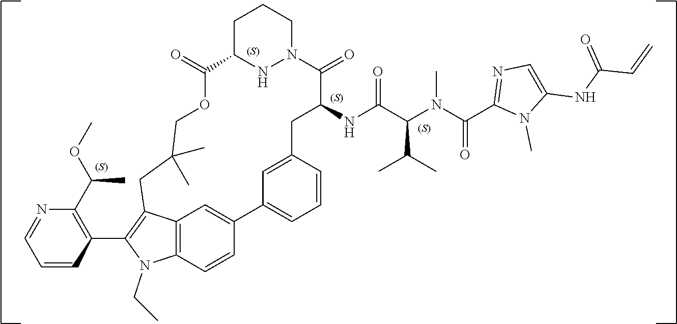 |
| BA525 | 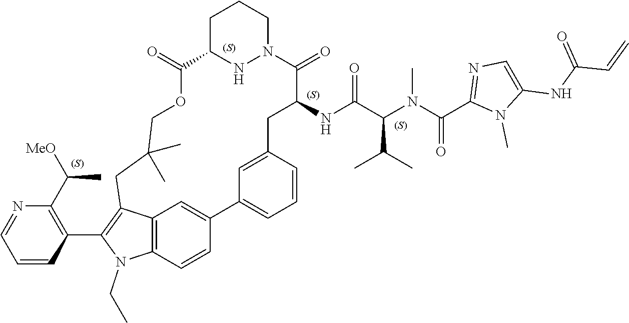 |
| BA526 | 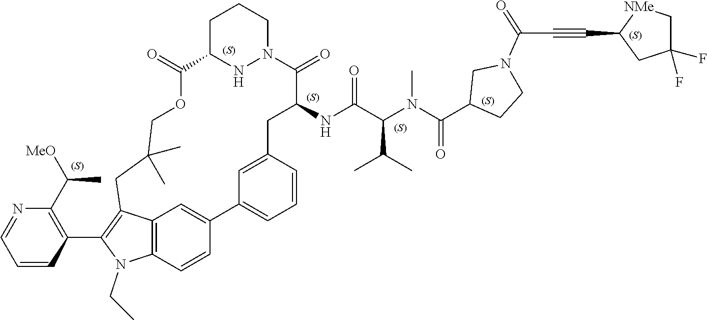 |
| BA527 | 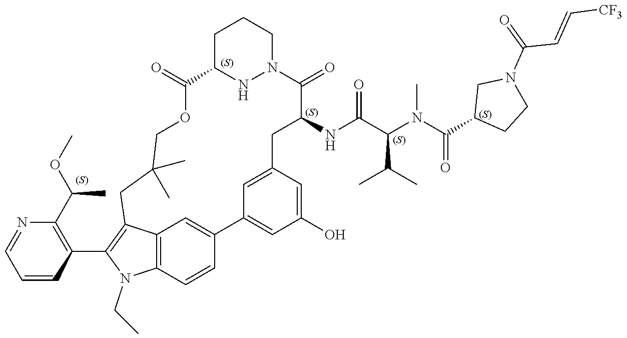 |
| BA528 | 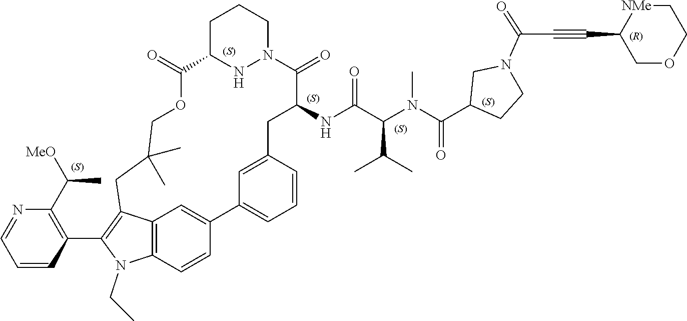 |
| BA529 | 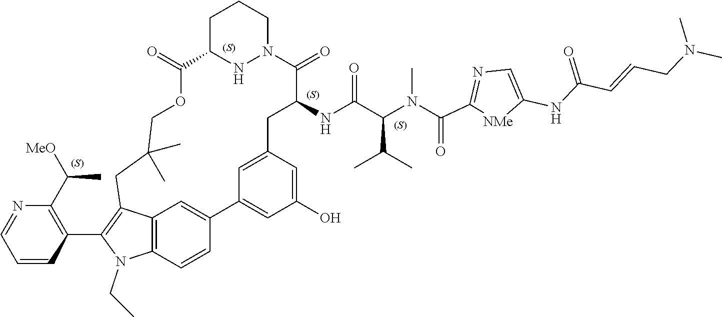 |
| BA530 | 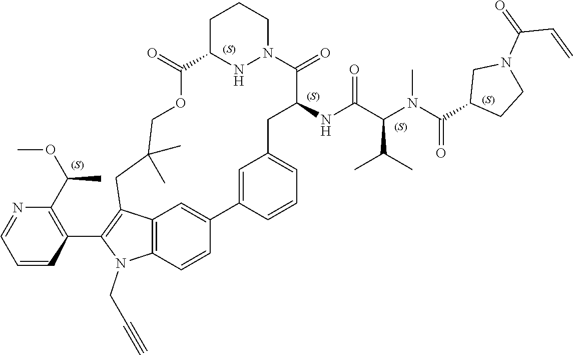 |
| BA531 | 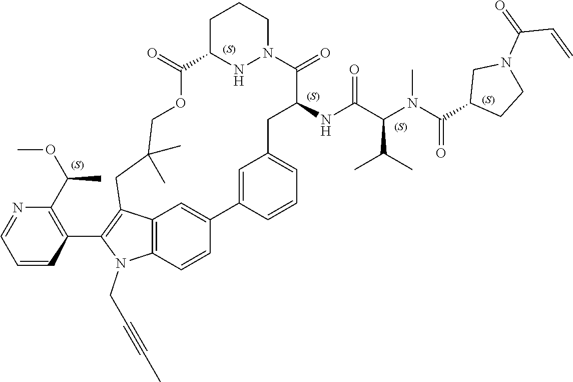 |
| BA532 | 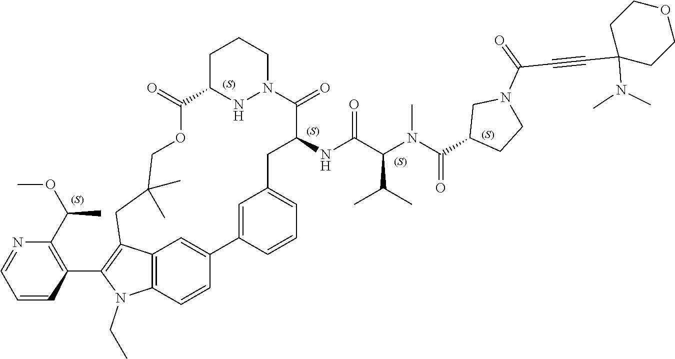 |
| BA533 | 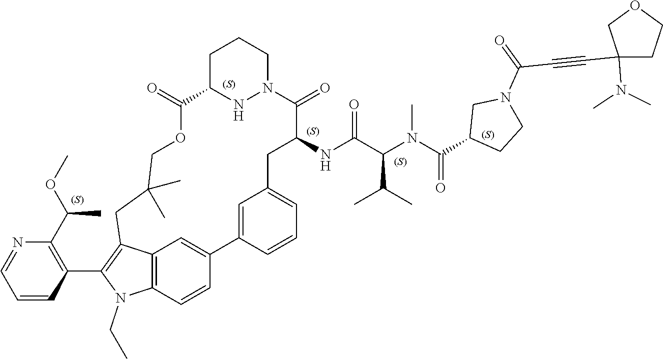 |
| BA534 | 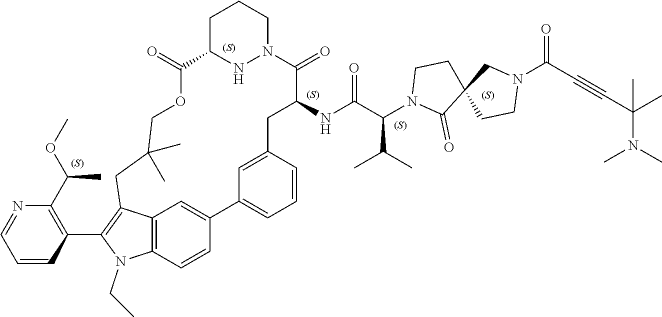 |
| BA535 | 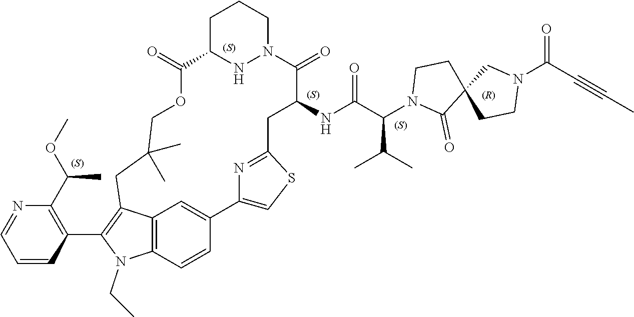 |
| BA536 | 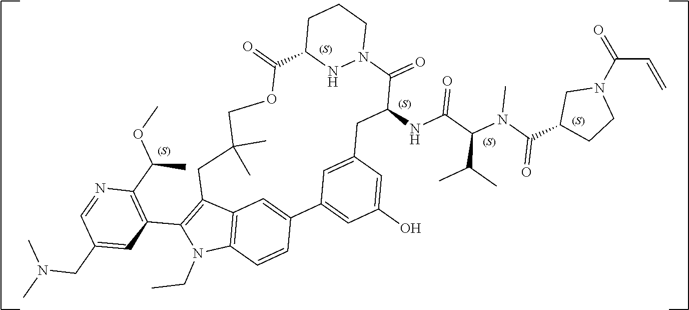 |
| BA537 | 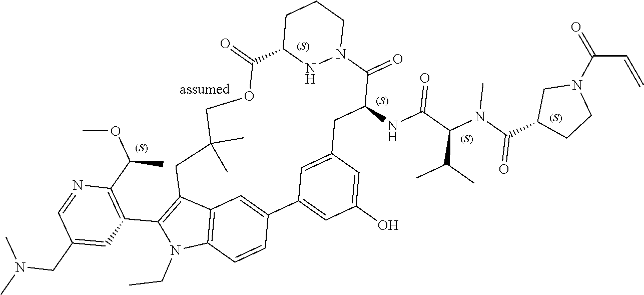 |
| BA538 | 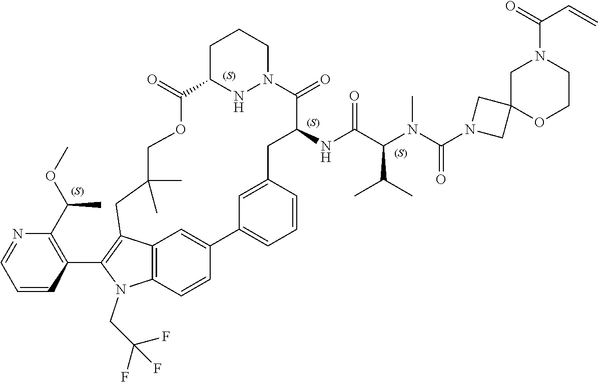 |
| BA539 | 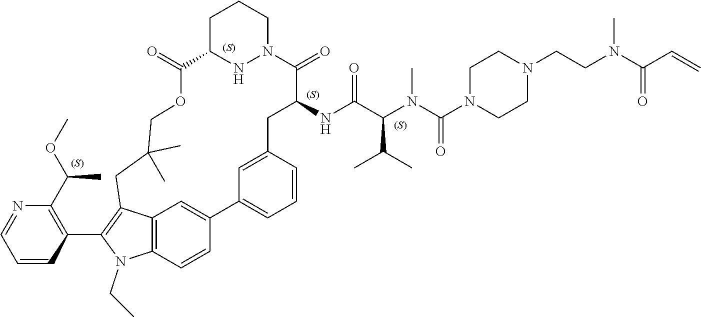 |
| BA540 | 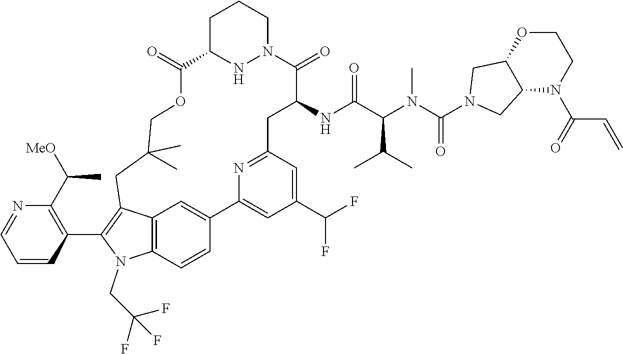 |
| BA541 | 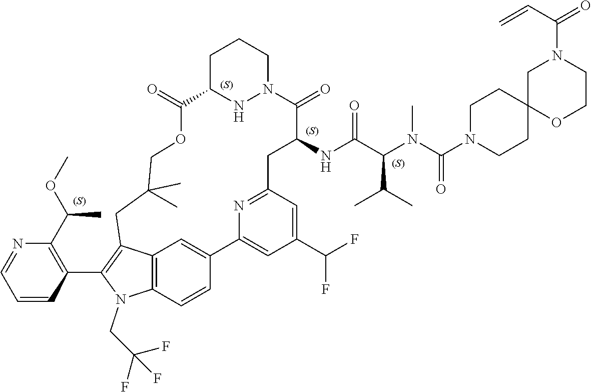 |
| BA542 | 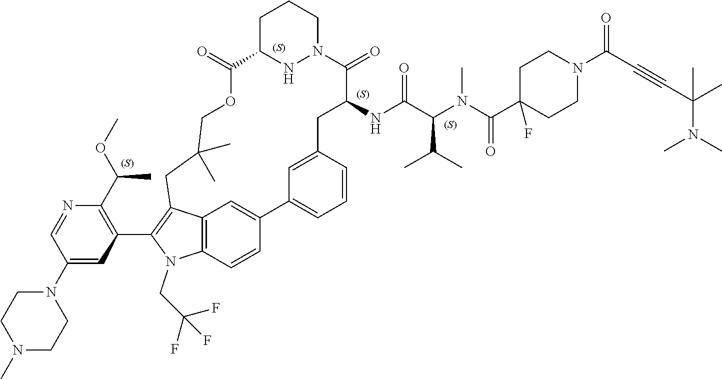 |
| BA543 | 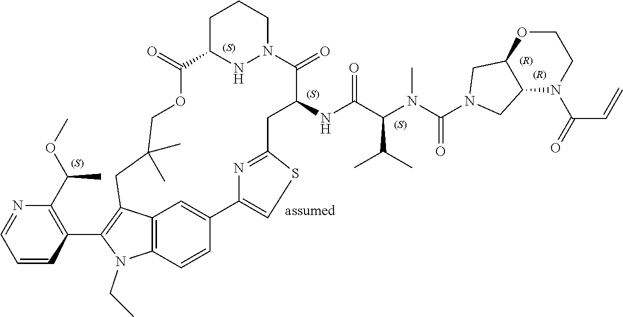 |
| BA544 | 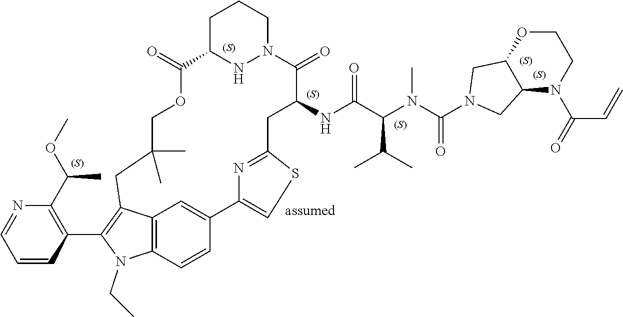 |
| BA545 | 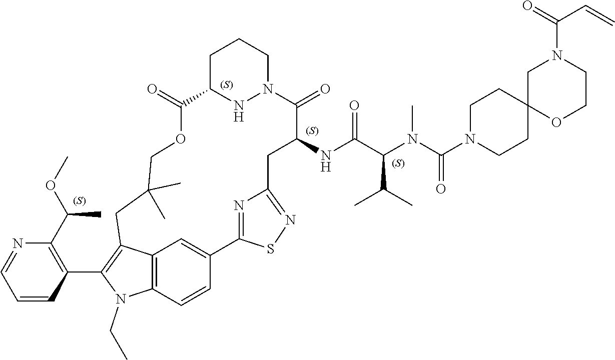 |
| BA546 | 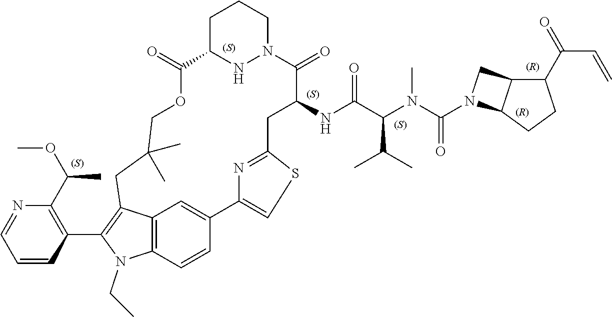 |
| BA547 | 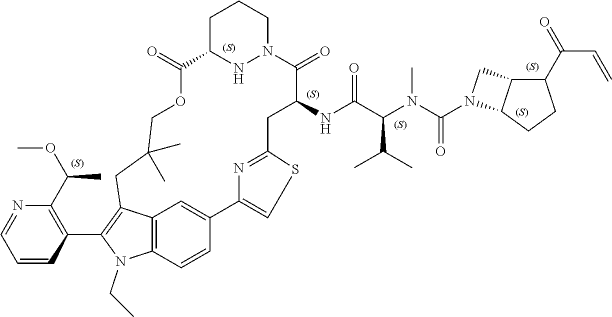 |
| BA548 | 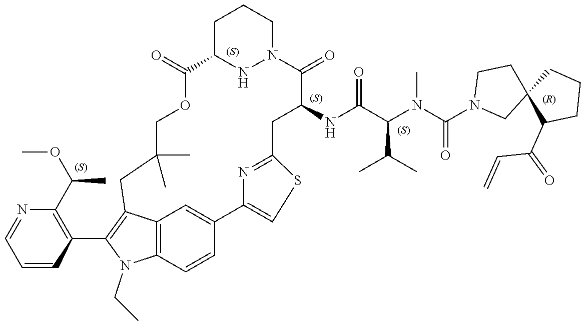 |
| BA549 | 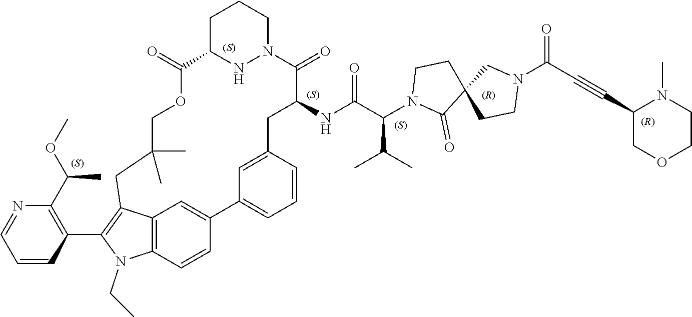 |
| BA550 | 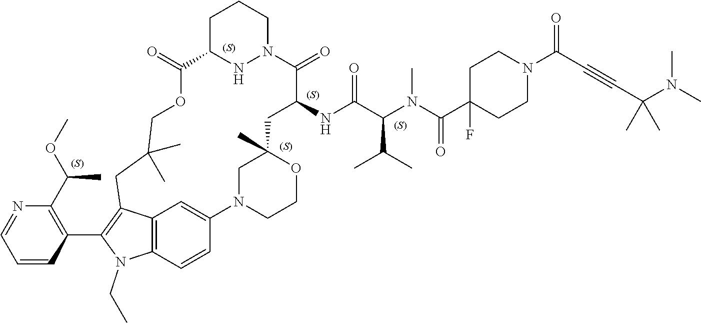 |
| BA551 | 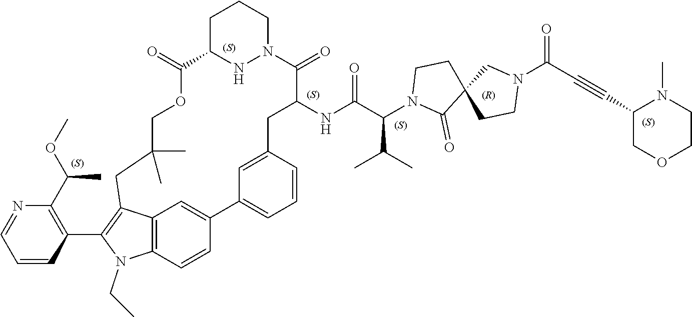 |
| BA552 | 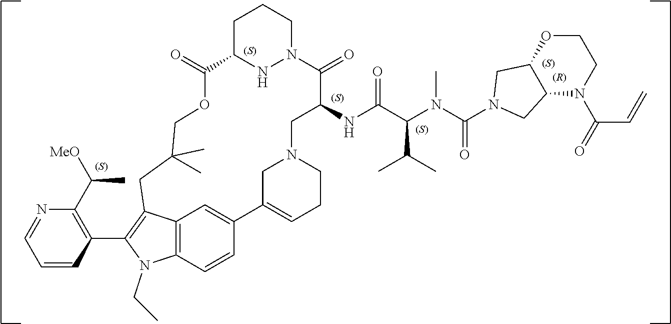 |
| BA553 | 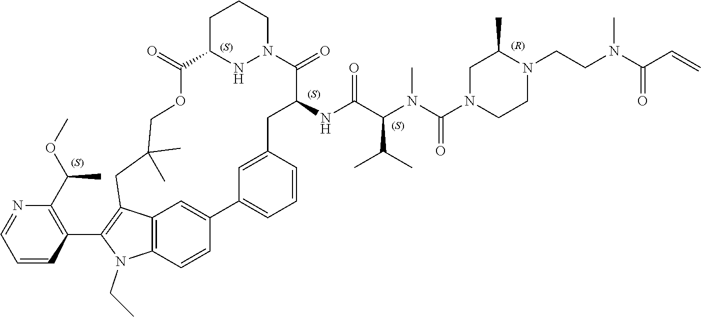 |
| BA554 | 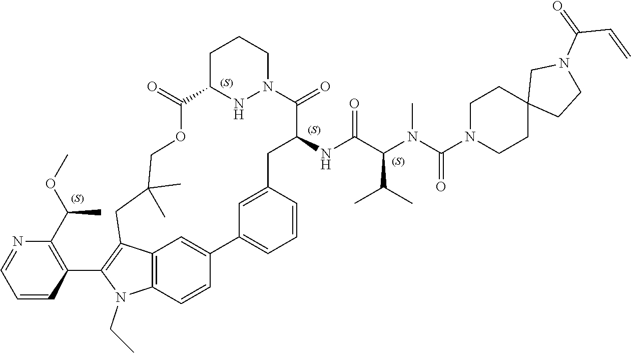 |
| BA555 | 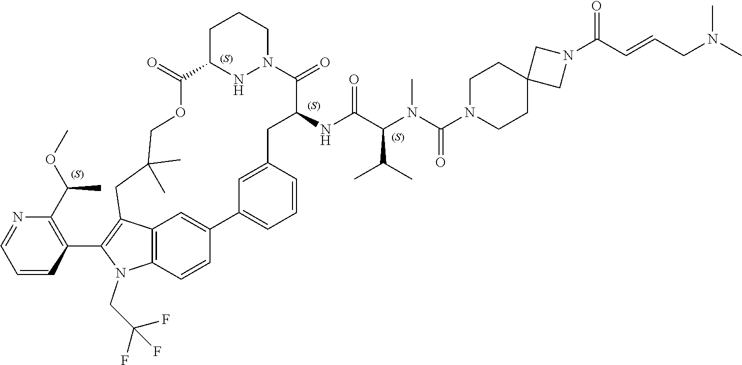 |
| BA556 | 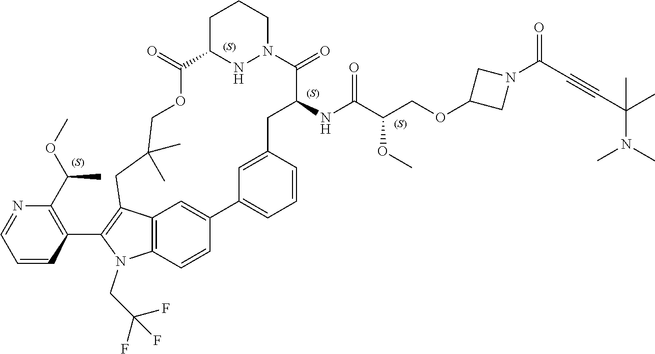 |
| BA557 | 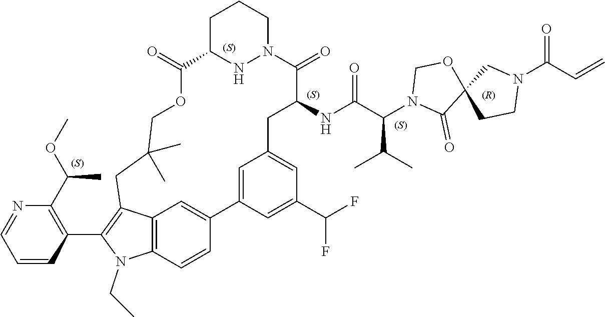 |
| BA558 | 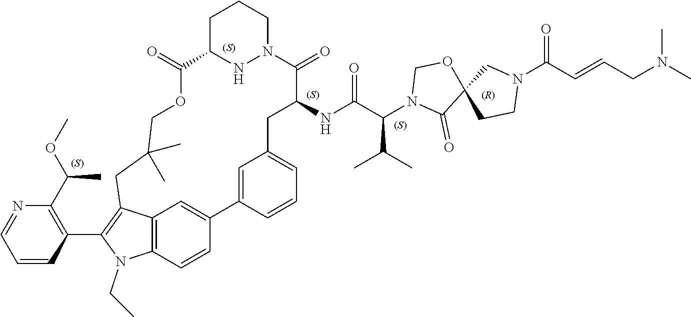 |
| BA559 | 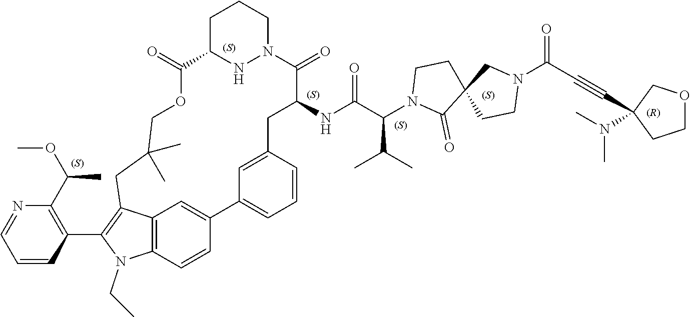 |
| BA560 | 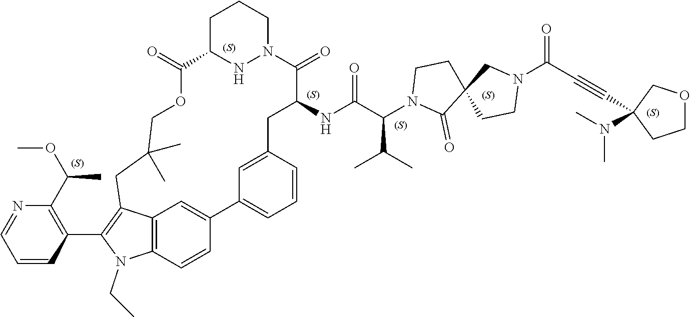 |
| BA561 | 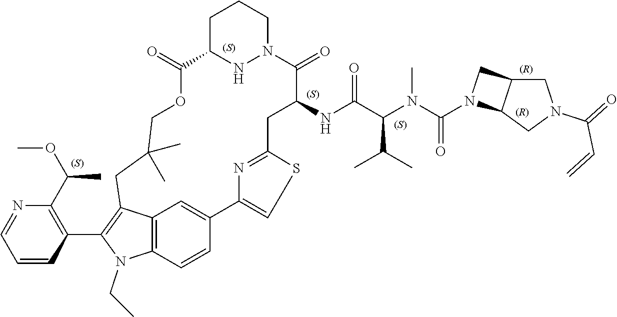 |
| BA562 | 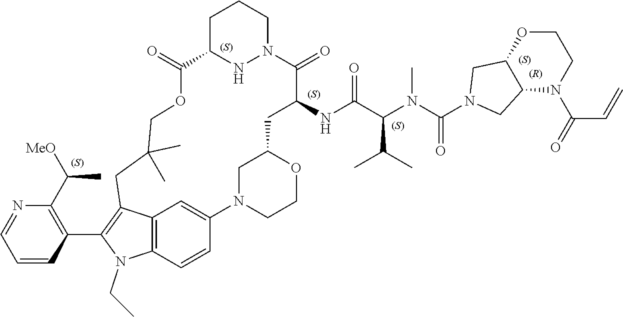 |
| BA563 | 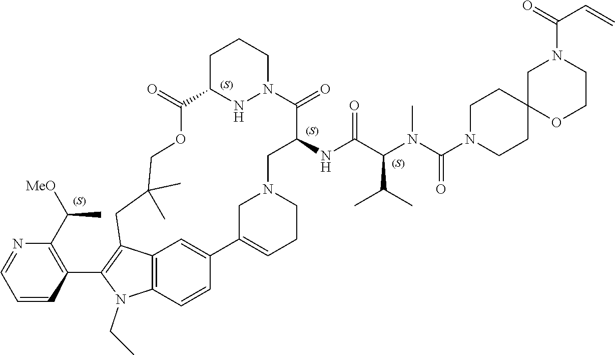 |
| BA564 | 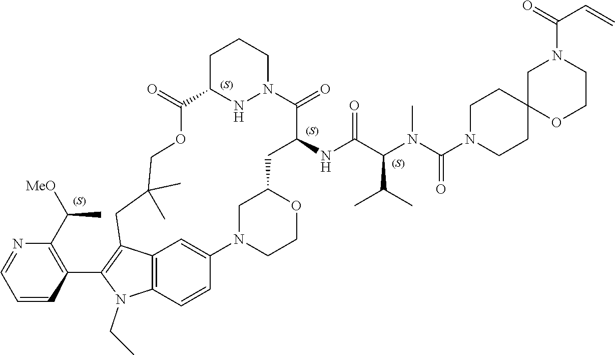 |
| BA565 | 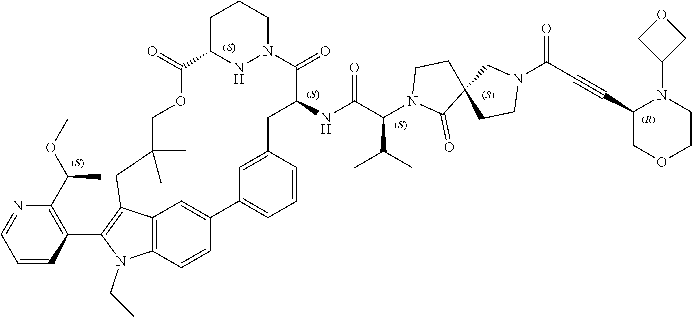 |
| BA566 | 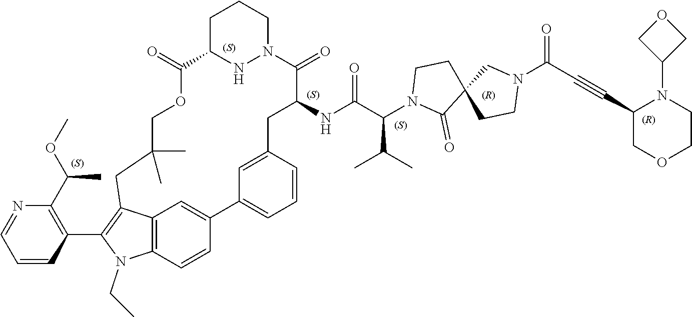 |
| BA567 | 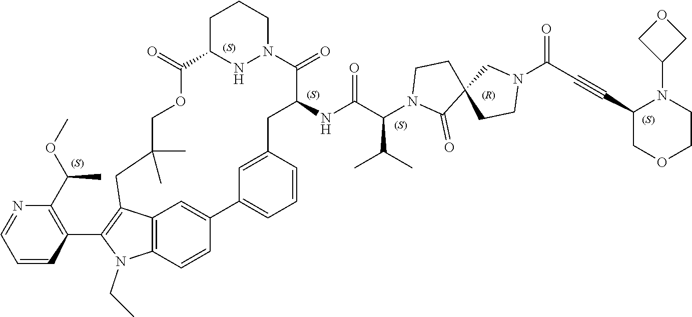 |
| BA568 | 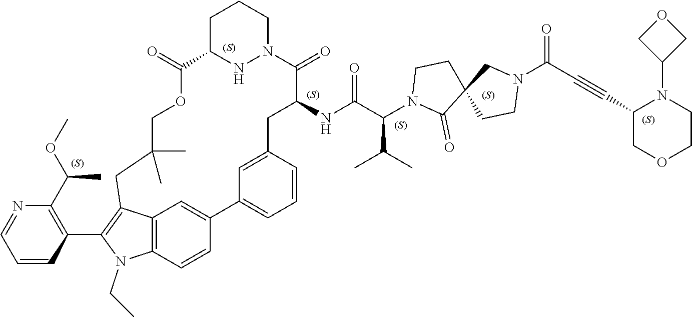 |
| BA569 | 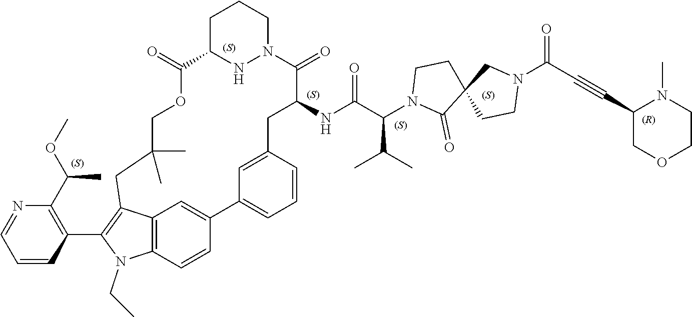 |
| BA570 | 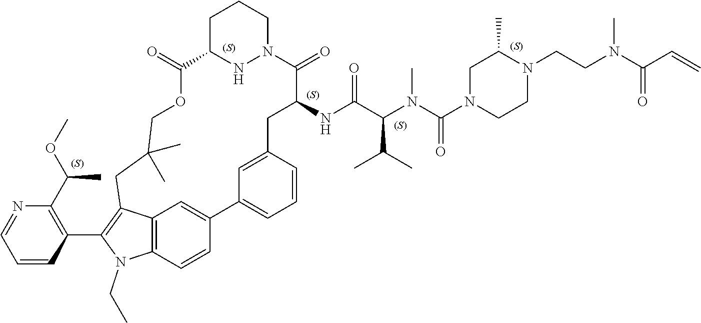 |
| BA571 | 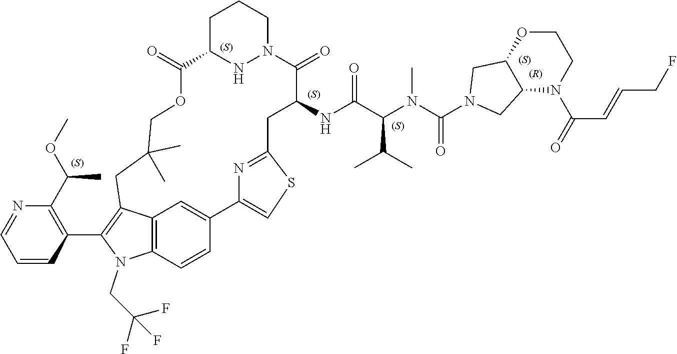 |
| BA572 | 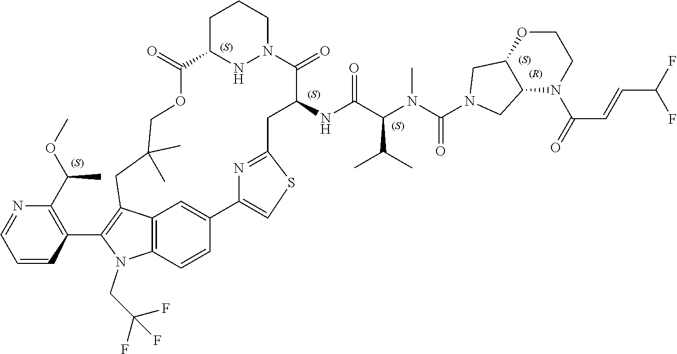 |
| BA573 | 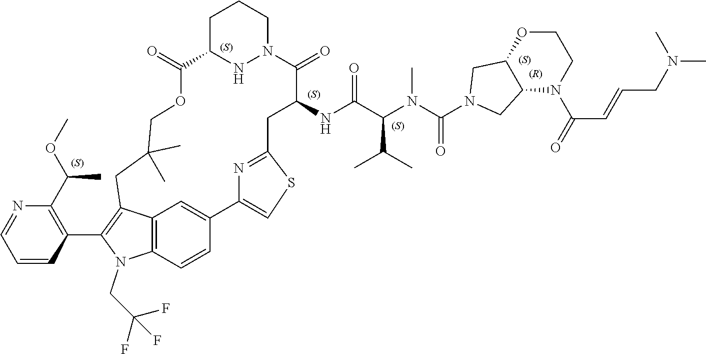 |
| BA574 | 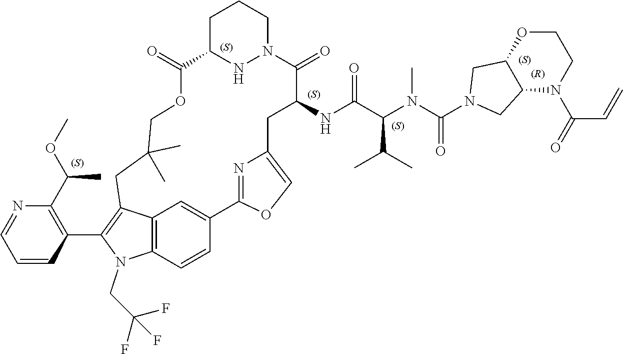 |
| BA575 | 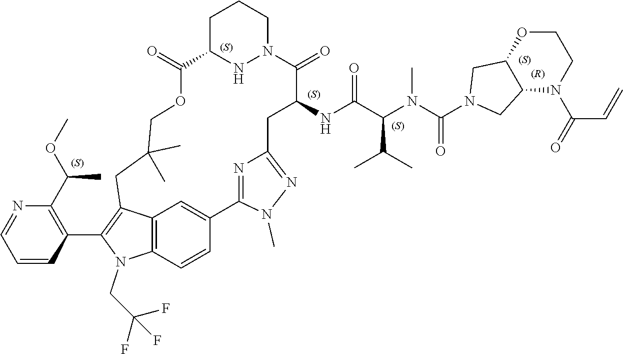 |
| BA576 | 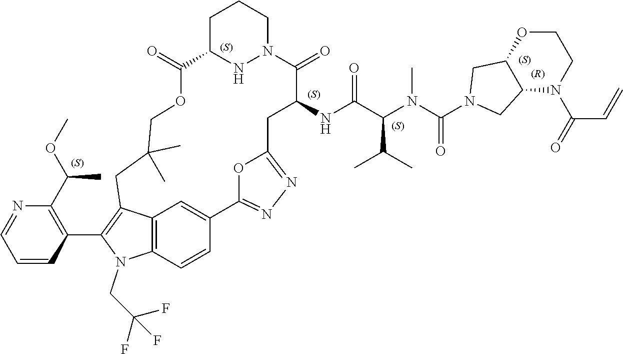 |
| BA577 | 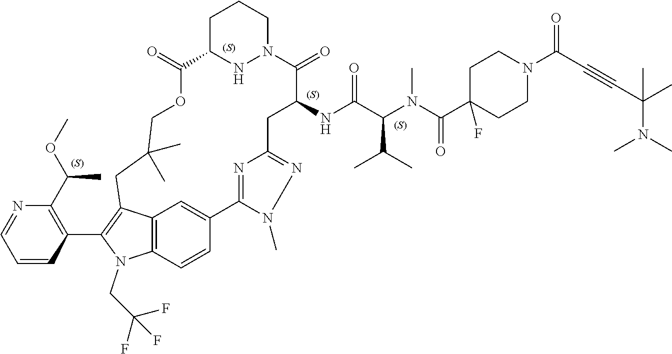 |
| BA578 | 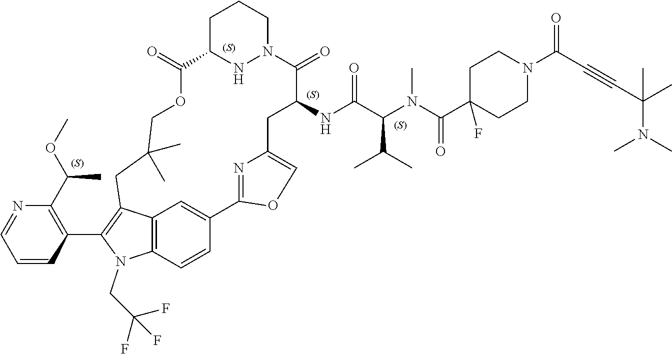 |
| BA579 | 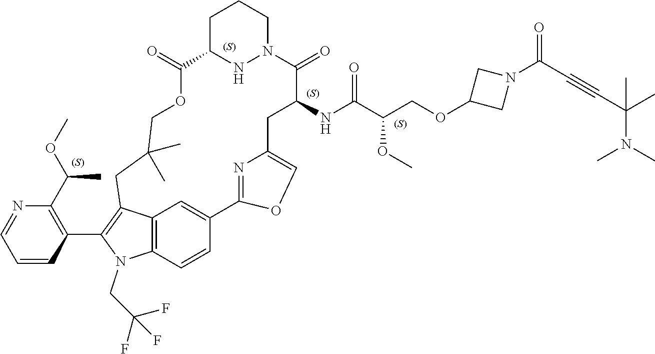 |
| BA580 | 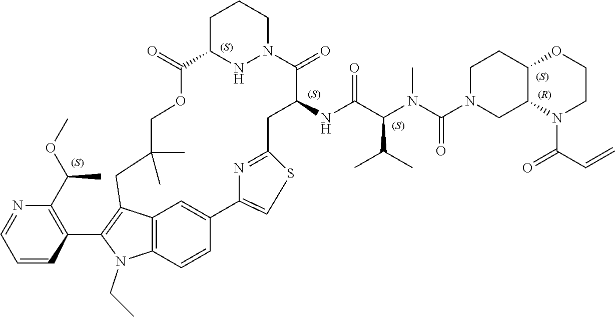 |
| BA581 | 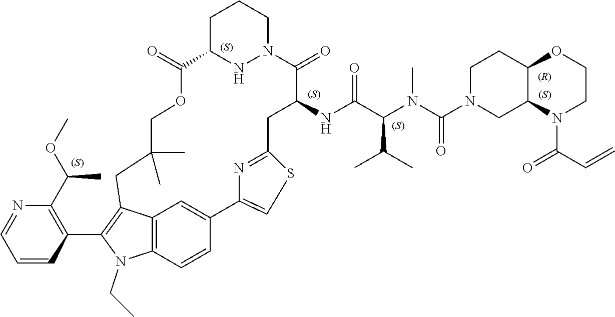 |
| BA582 | 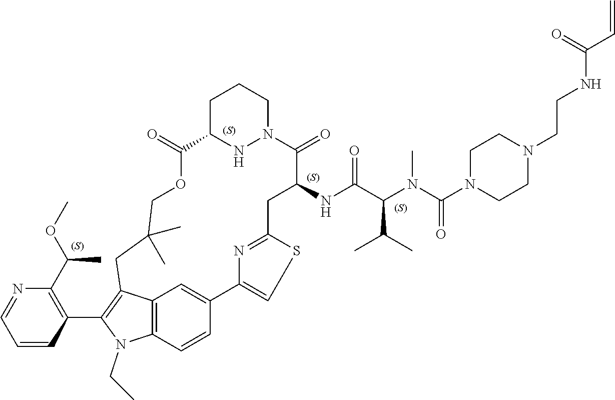 |
| BA583 | 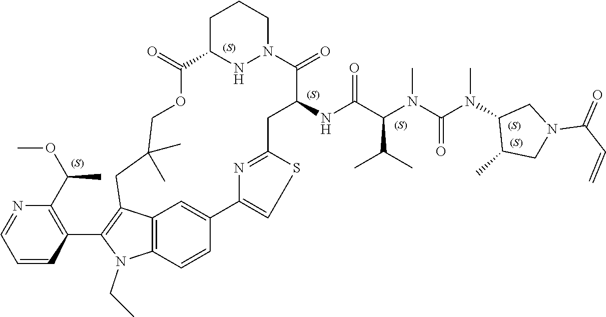 |
| BA584 | 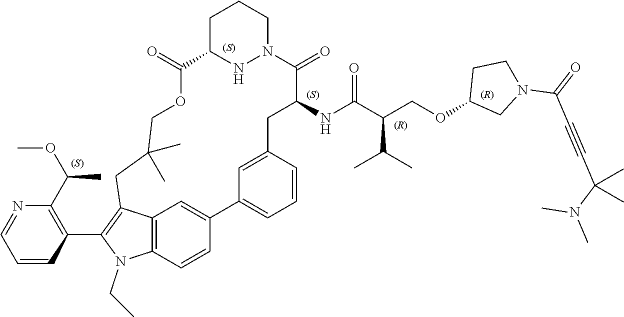 |
| BA585 | 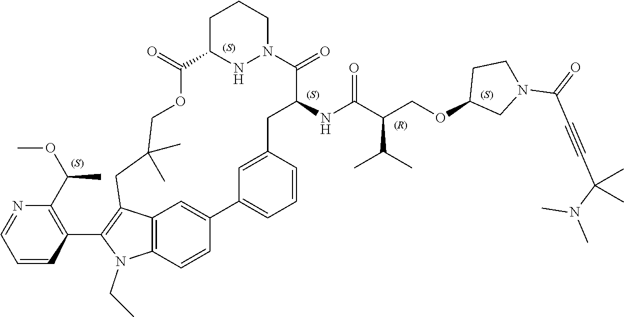 |
| BA586 | 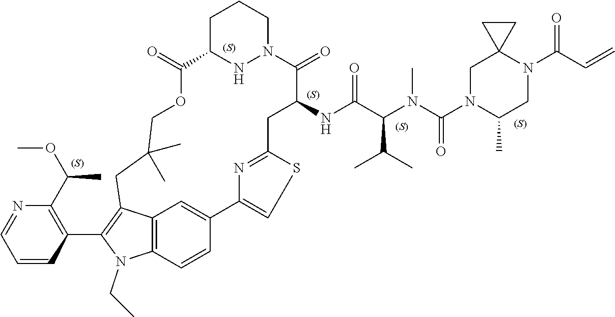 |
| BA587 | 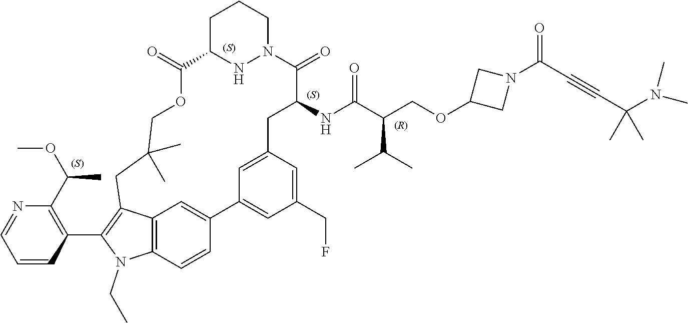 |
| BA588 | 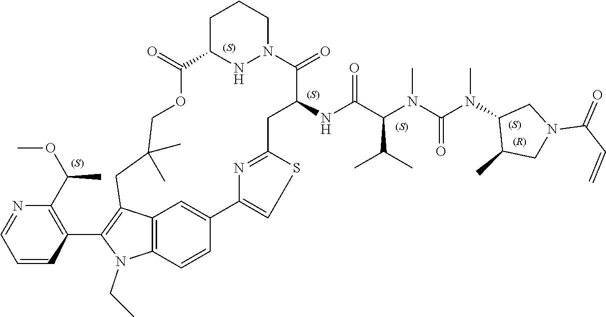 |
| BA589 | 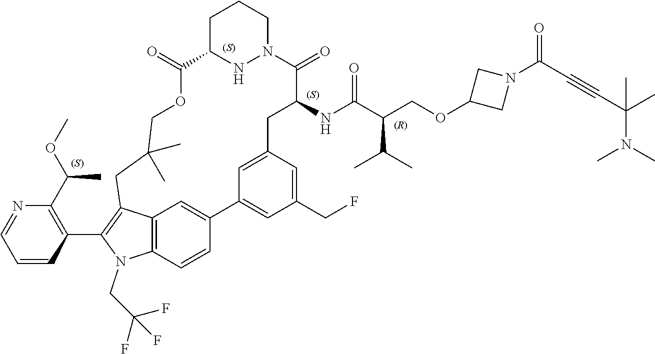 |
| BA590 | 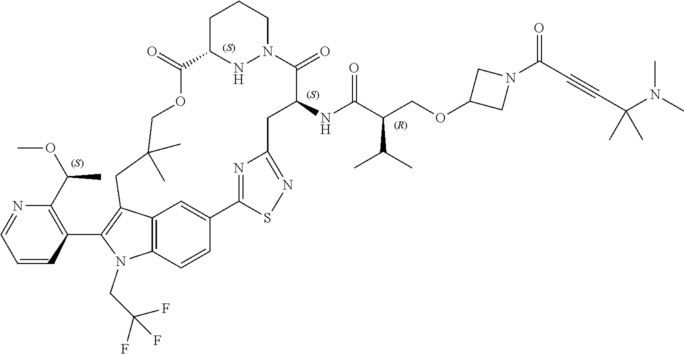 |
| BA591 | 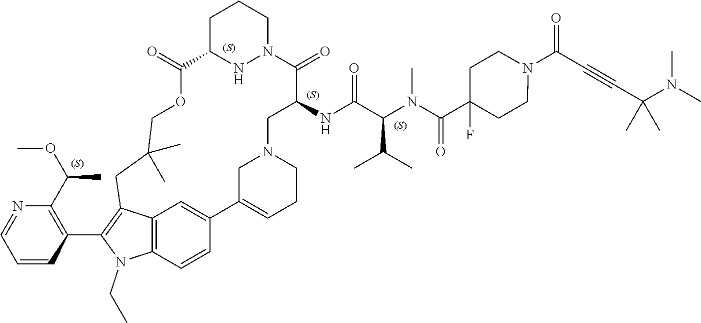 |
| BA592 | 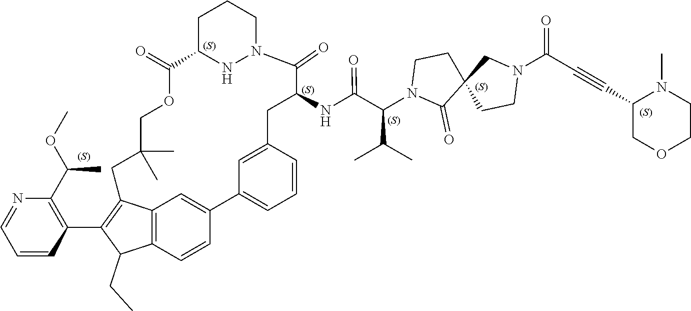 |
| BA593 | 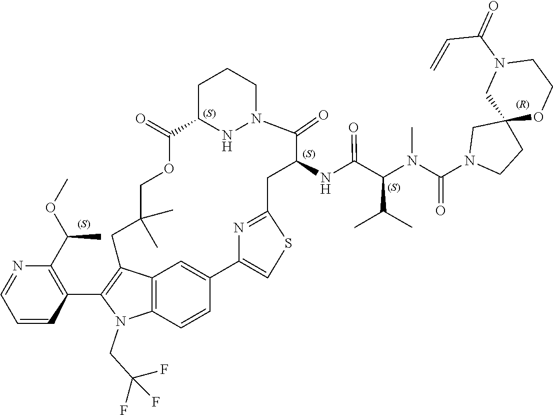 |
| BA594 | 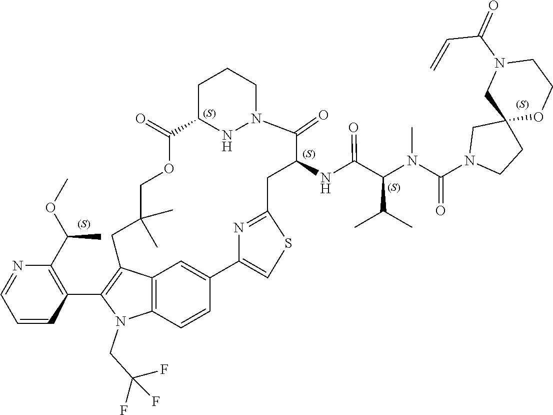 |
| BA595 | 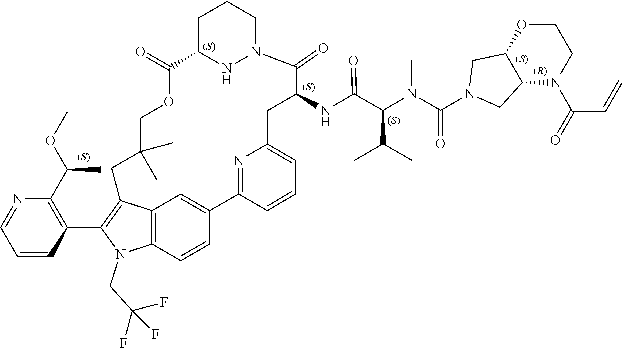 |
| BA596 | 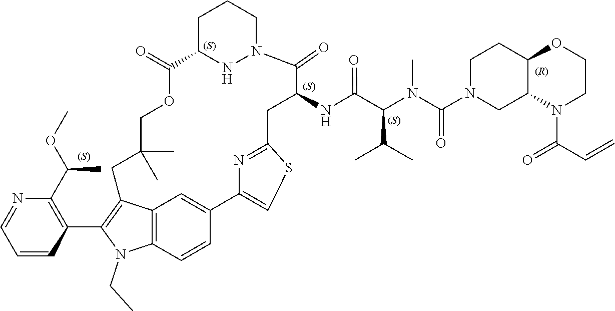 |
| BA597 | 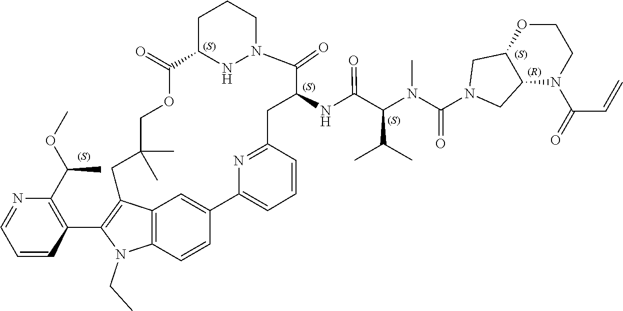 |
| BA598 | 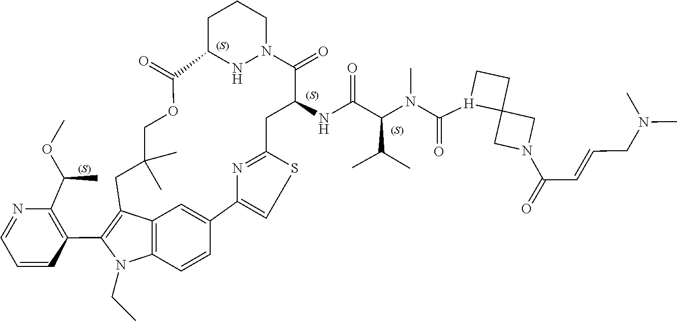 |
| BA599 | 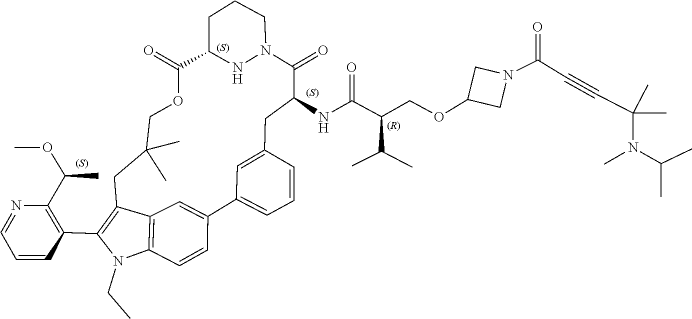 |
| BA600 | 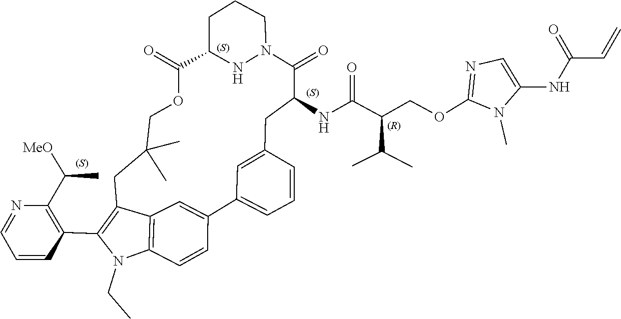 |
| BA601 | 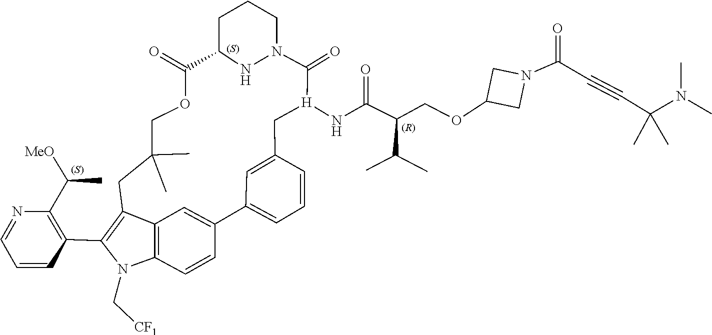 |
| BA602 | 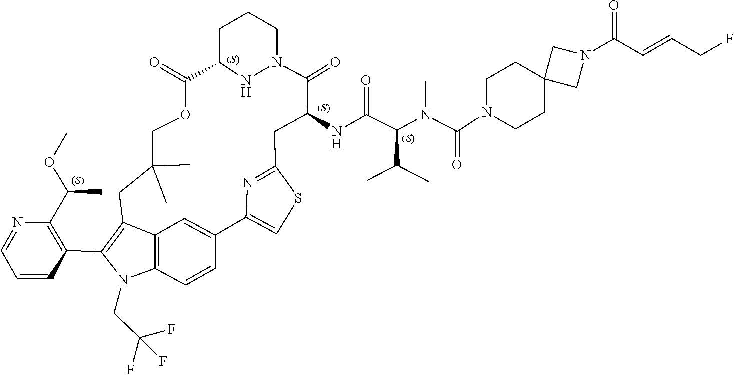 |
| BA603 | 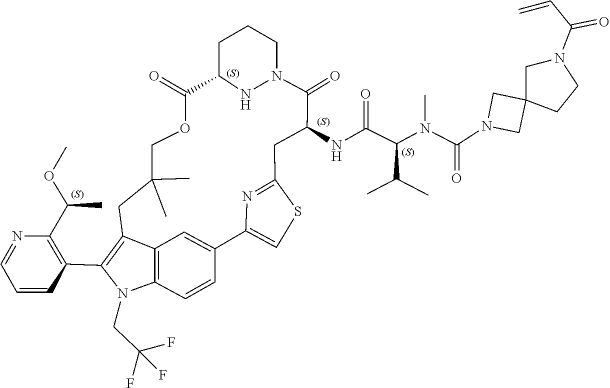 |
| BA604 | 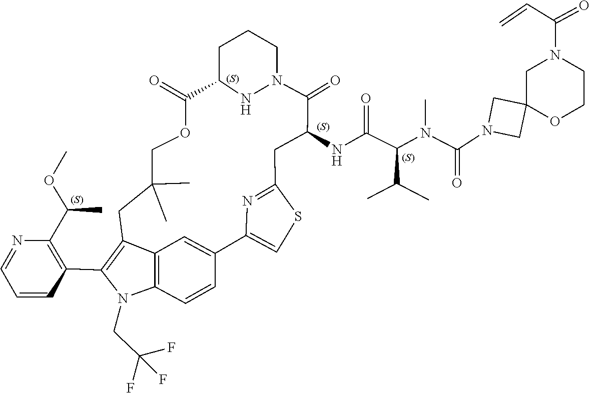 |
| BA605 | 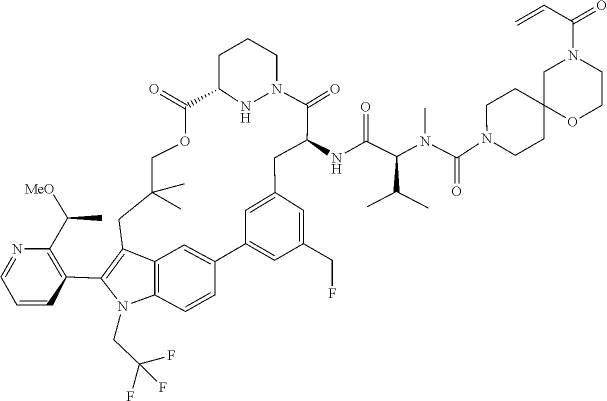 |
| BA606 | 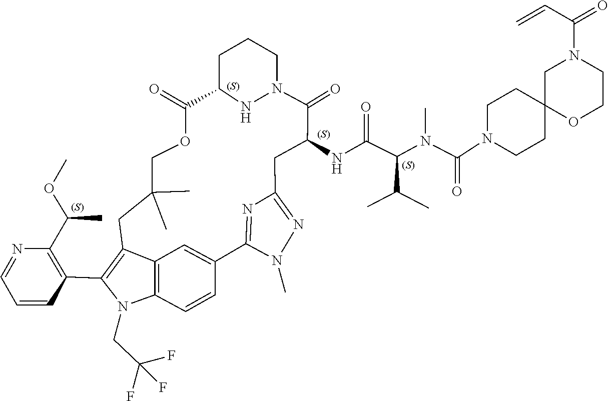 |
| BA607 | 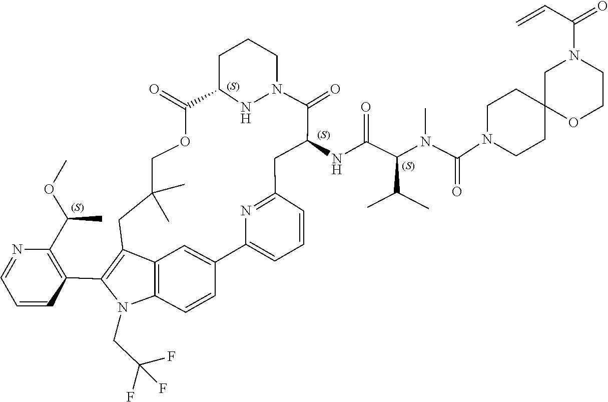 |
| BA608 | 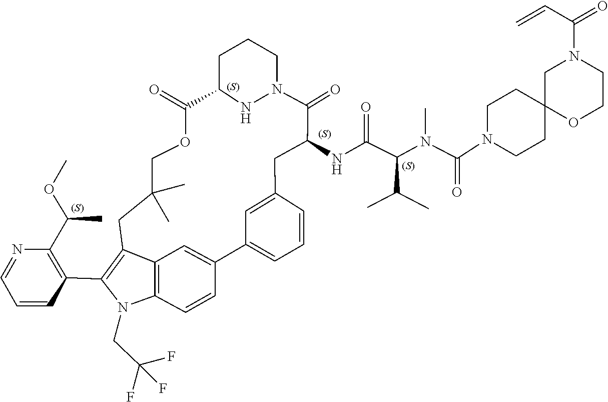 |
| BA609 | 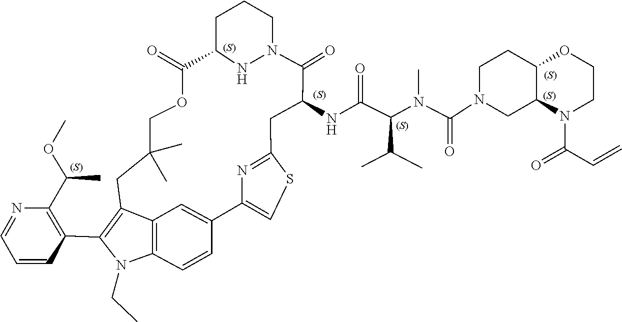 |
| BA610 | 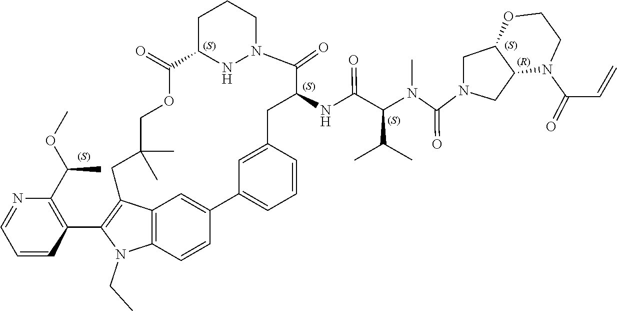 |
| BA611 | 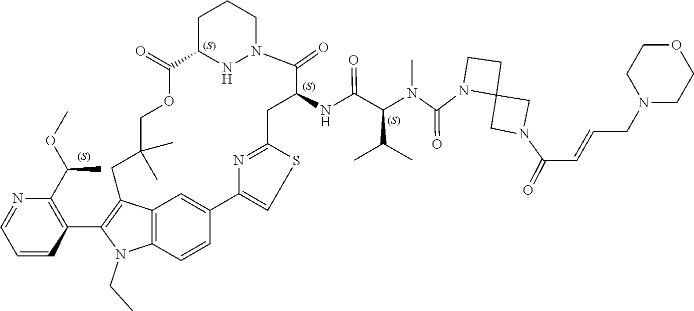 |
| BA612 | 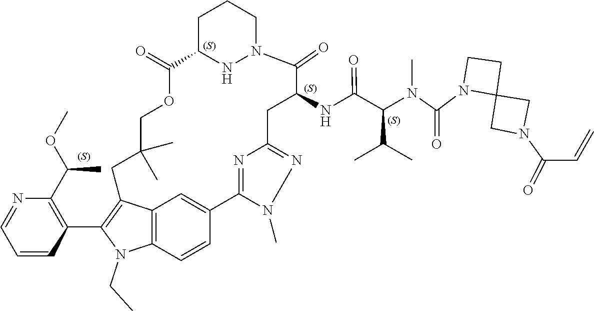 |
| BA613 | 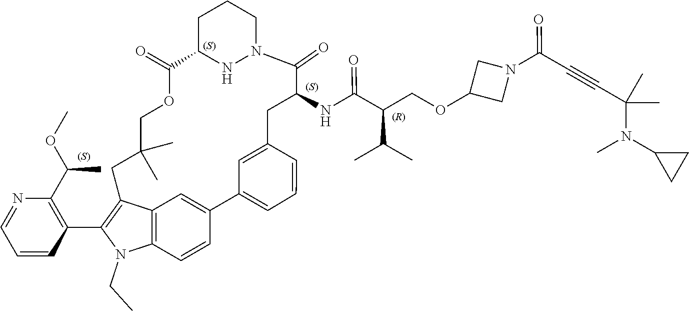 |
| BA614 | 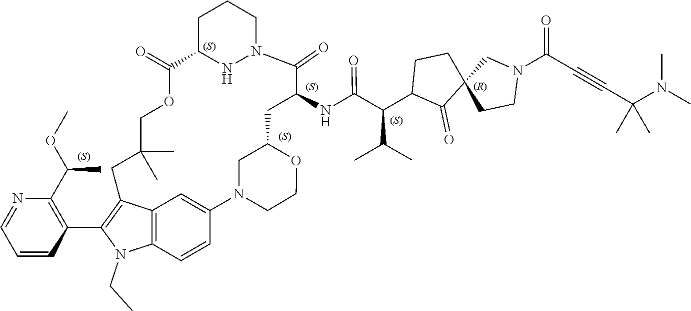 |
| BA615 | 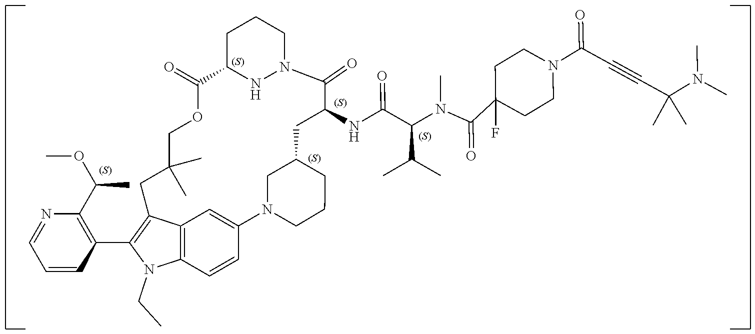 |
| BA616 | 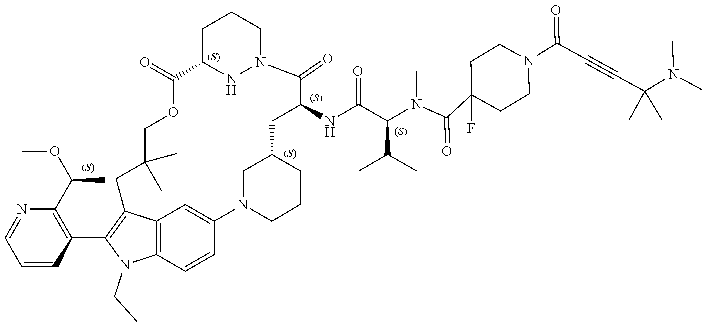 |
| BA617 | 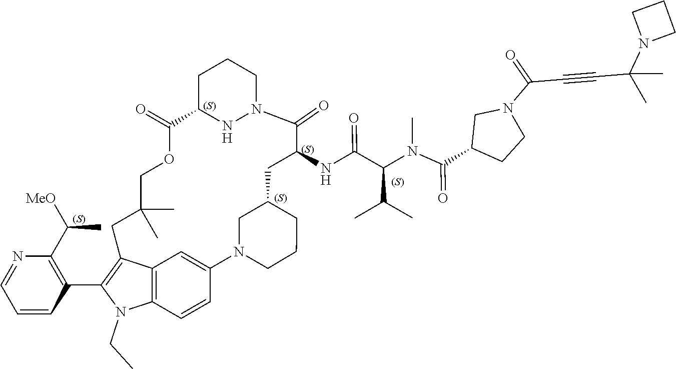 |
| BA618 | 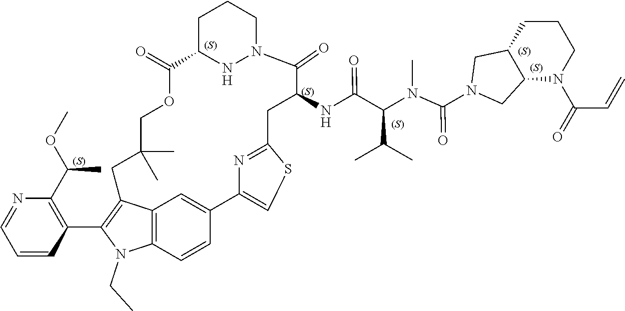 |
| BA619 | 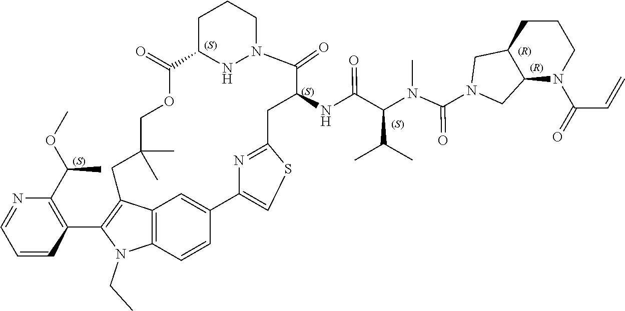 |
| BA620 | 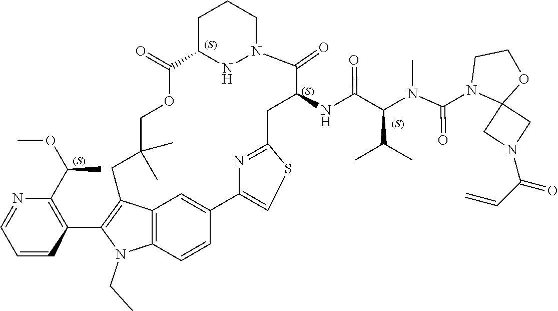 |
| BA621 | 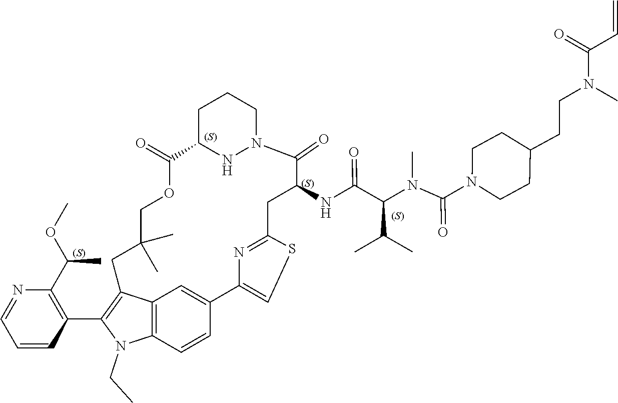 |
| BA622 | 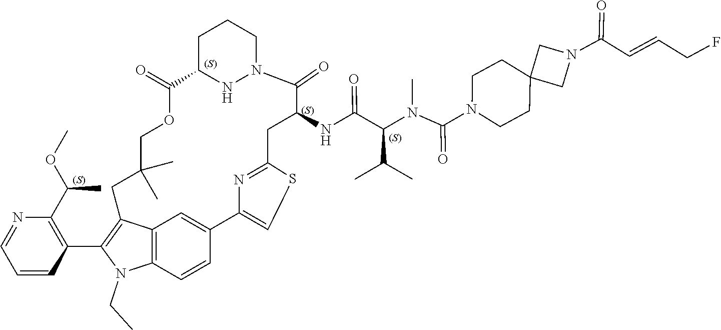 |
| BA623 | 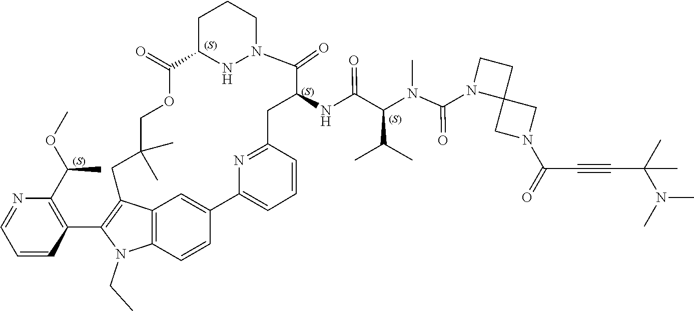 |
| BA624 | 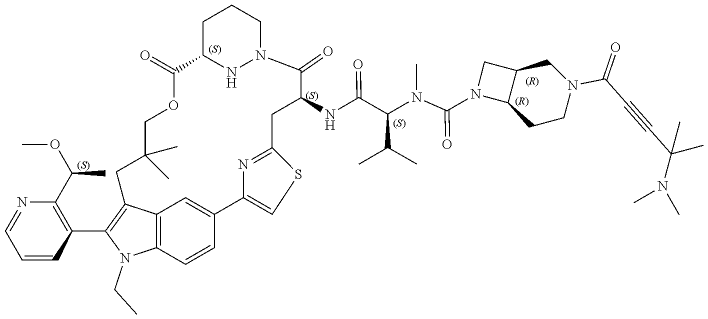 |
| BA625 | 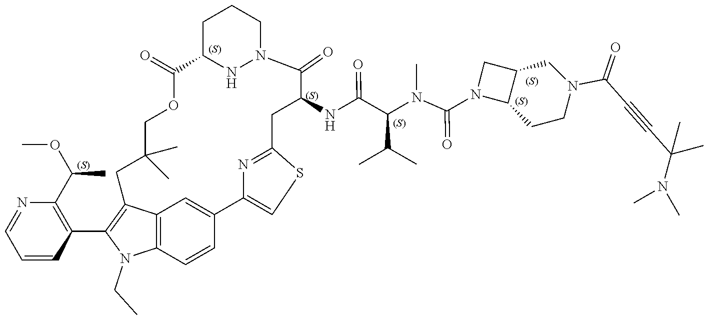 |
| BA626 | 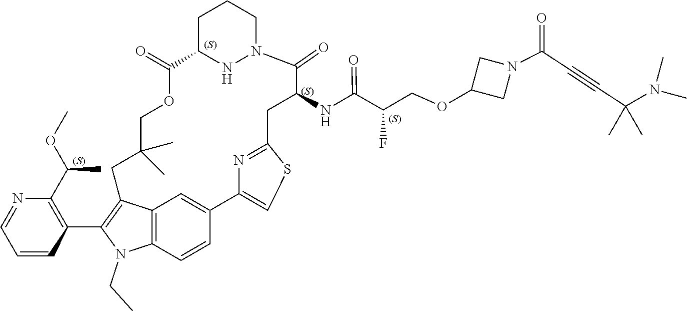 |
| BA627 | 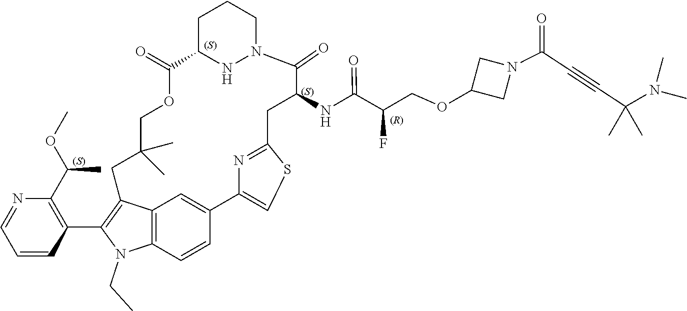 |
| BA628 | 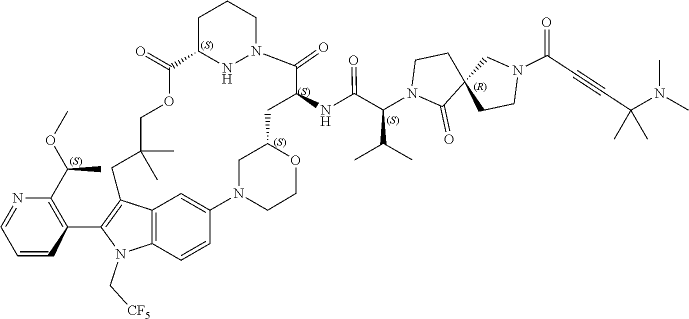 |
| BA629 | 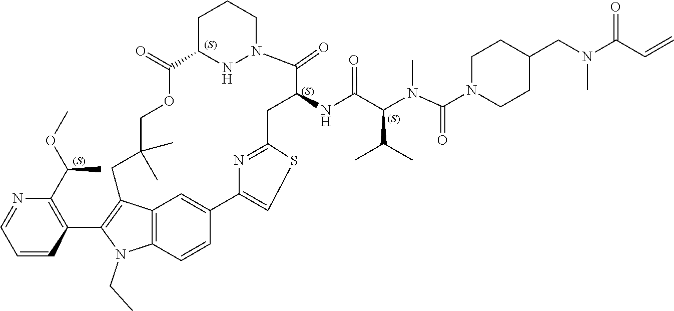 |
| BA630 | 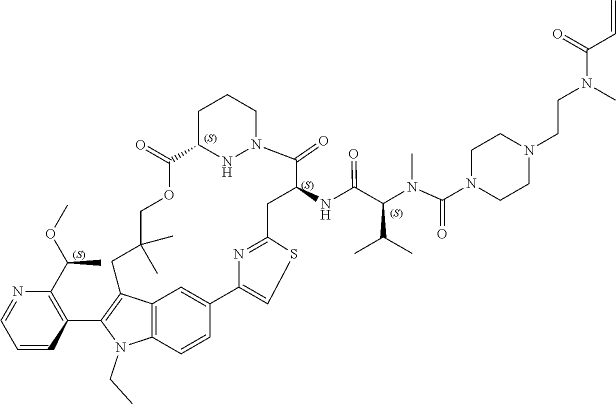 |
| BA631 | 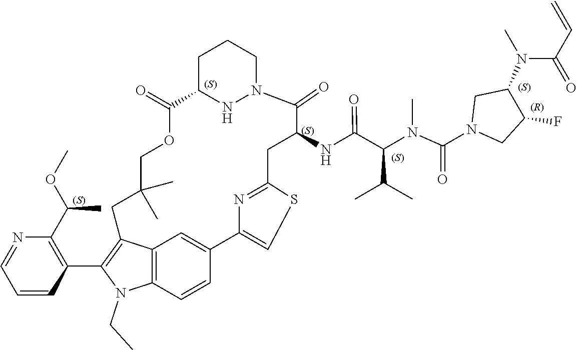 |
| BA632 | 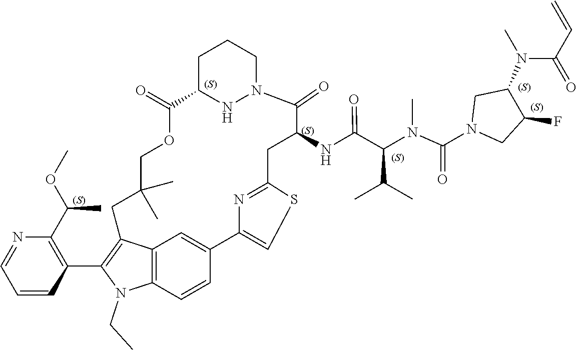 |
| BA633 | 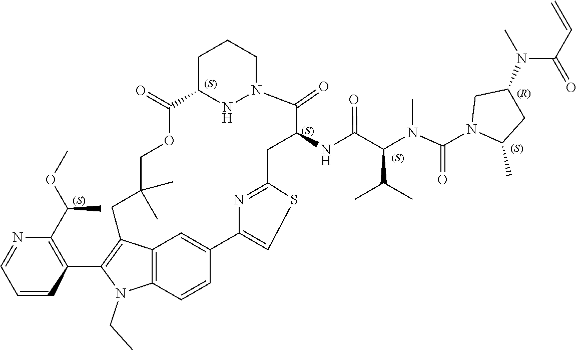 |
| BA634 | 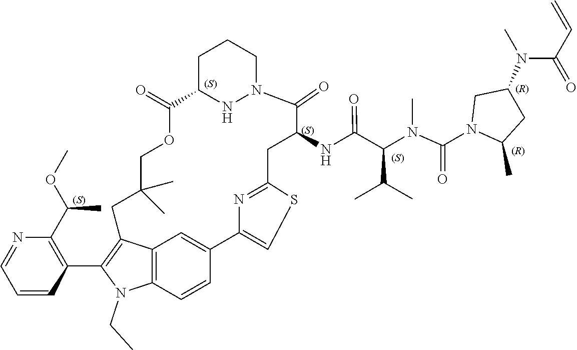 |
| BA635 | 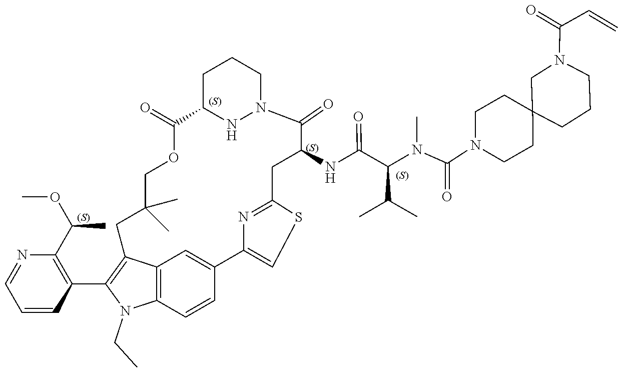 |
| BA636 | 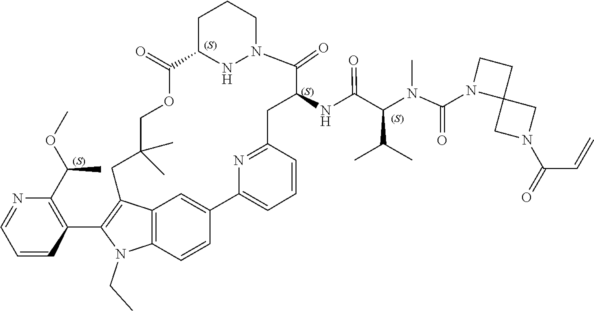 |
| BA637 | 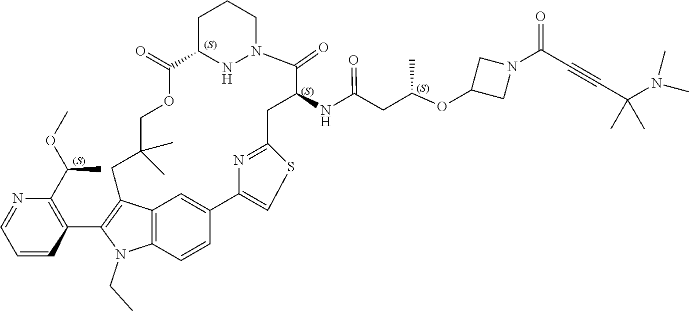 |
| BA638 | 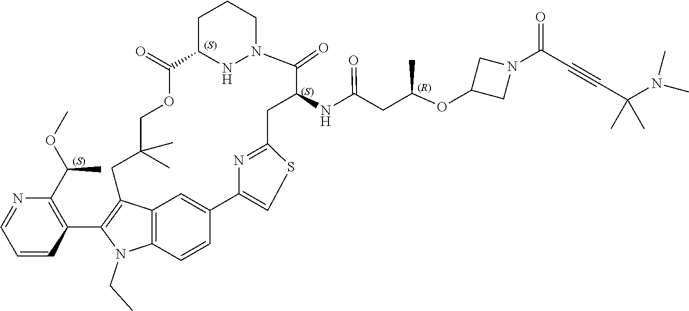 |
| BA639 | 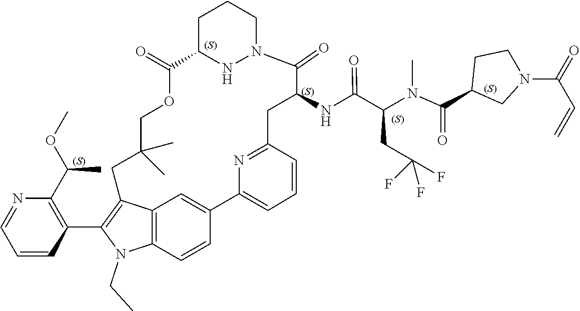 |
| BA640 | 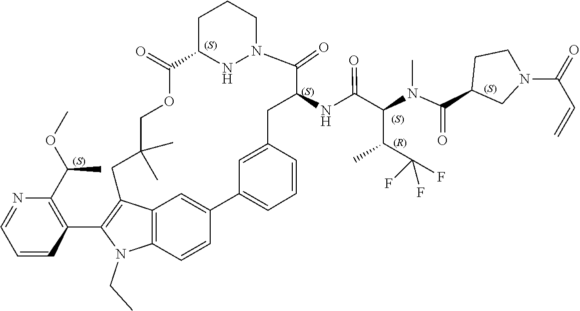 |
| BA641 | 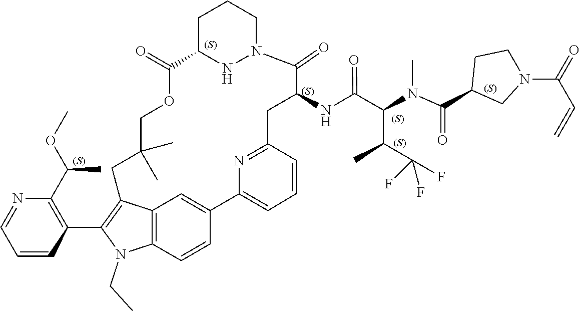 |
| BA642 | 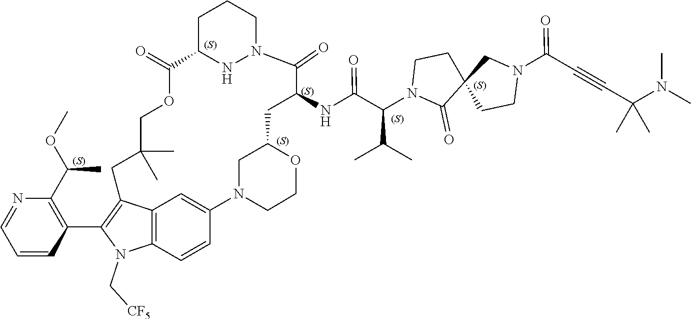 |
| BA643 | 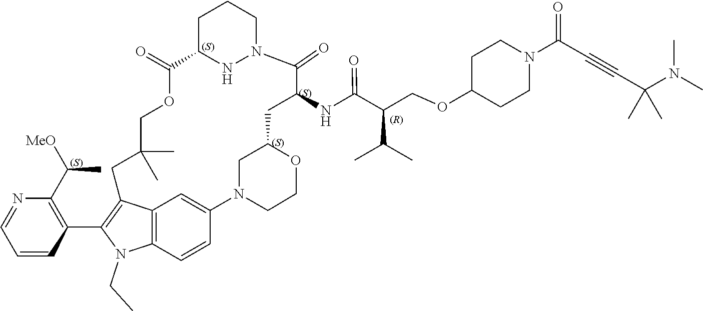 |
| BA644 | 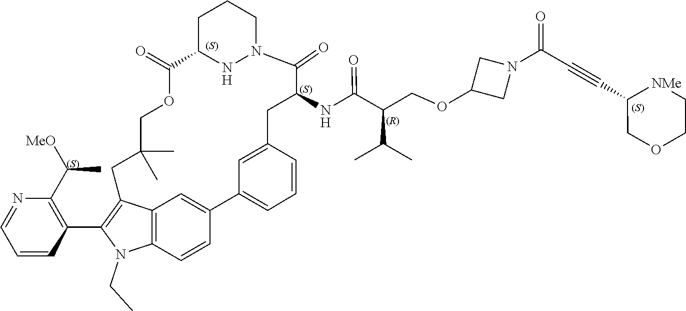 |
| BA645 | 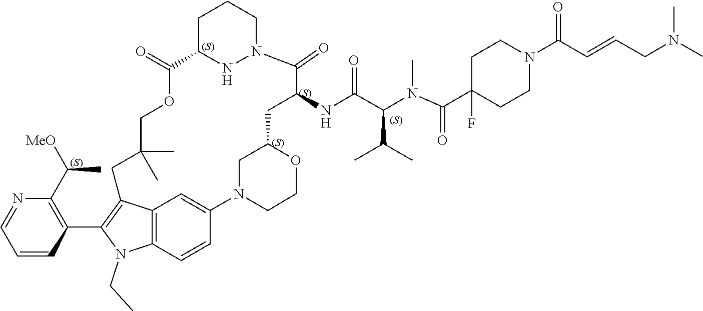 |
| BA646 | 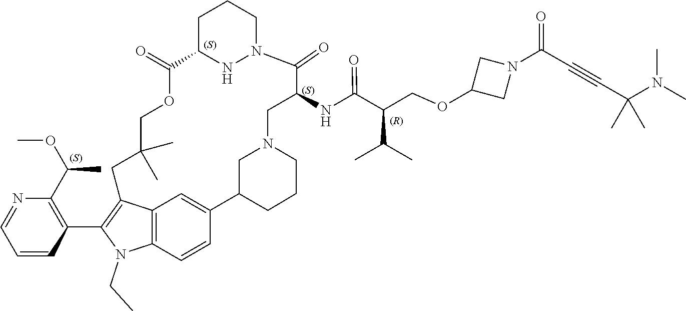 |
| BA647 | 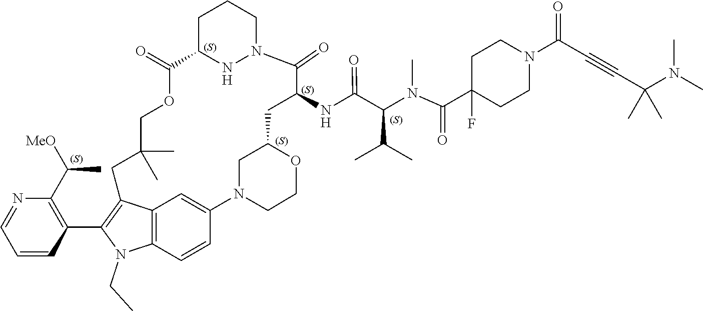 |
| BA648 | 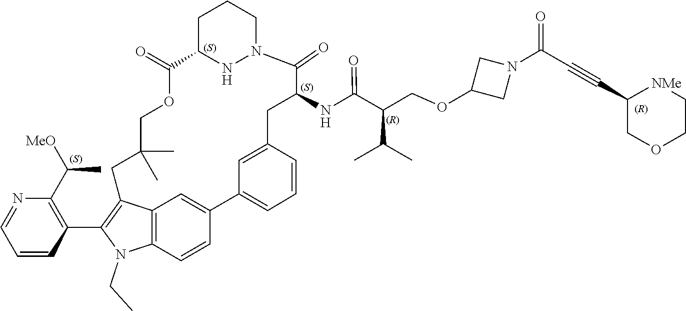 |
| BA649 | 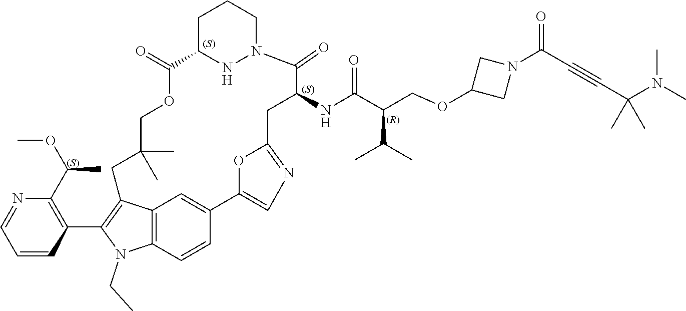 |
| BA650 | 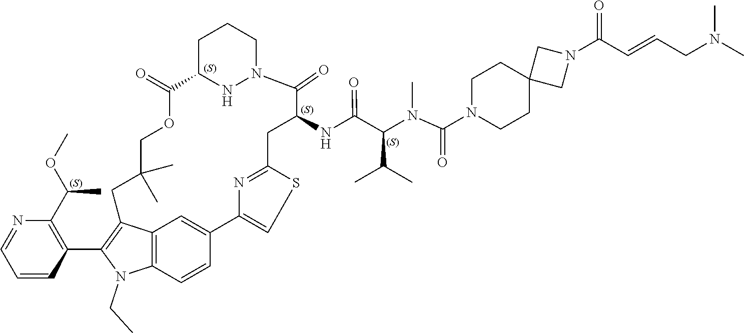 |
| BA651 | 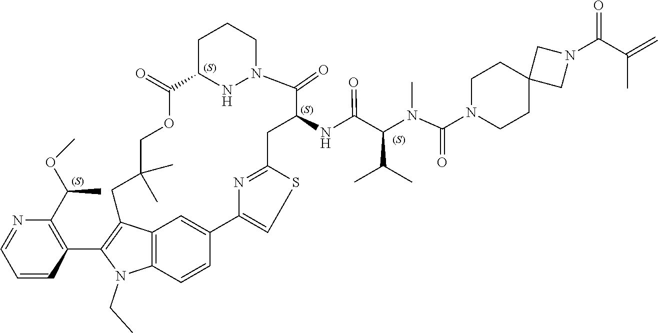 |
| BA652 | 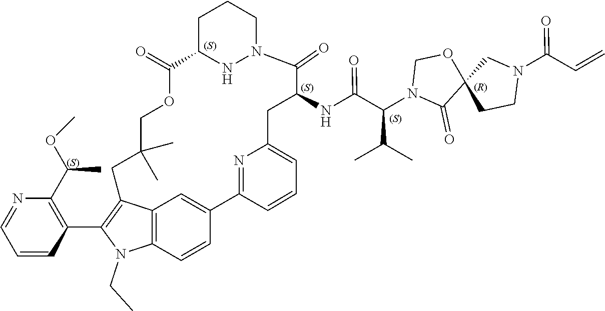 |
| BA653 | 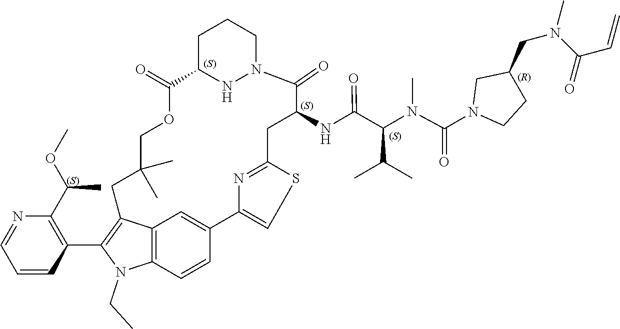 |
| BA654 | 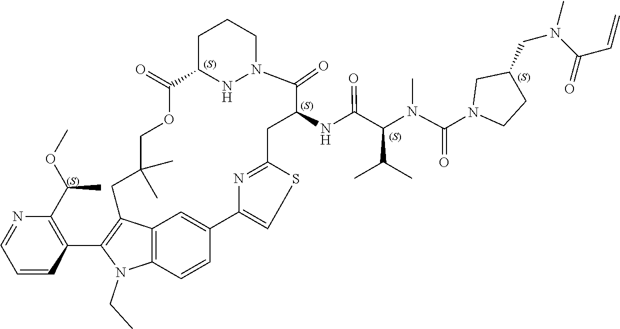 |
| BA655 | 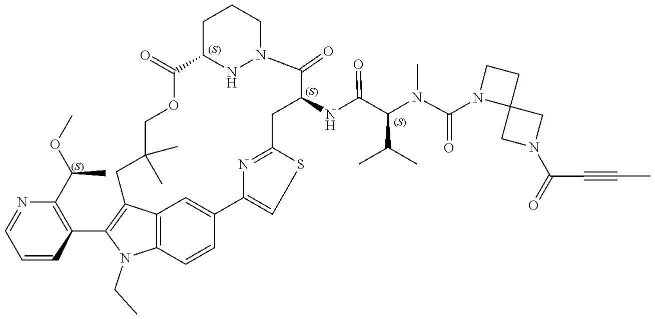 |
| BA656 | 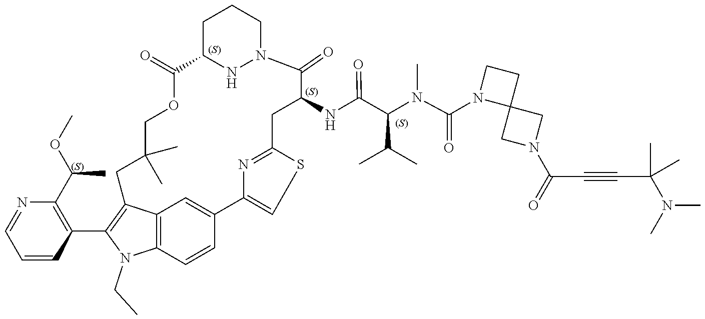 |
| BA657 | 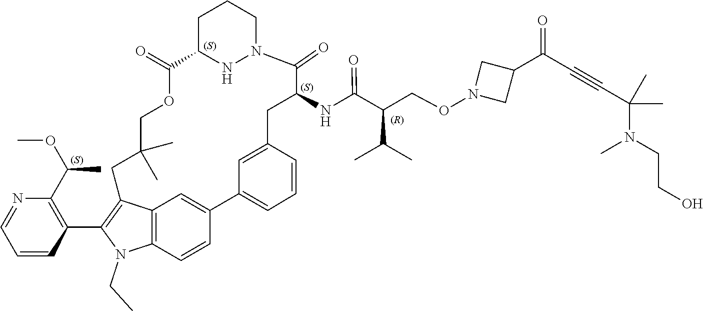 |
| BA658 | 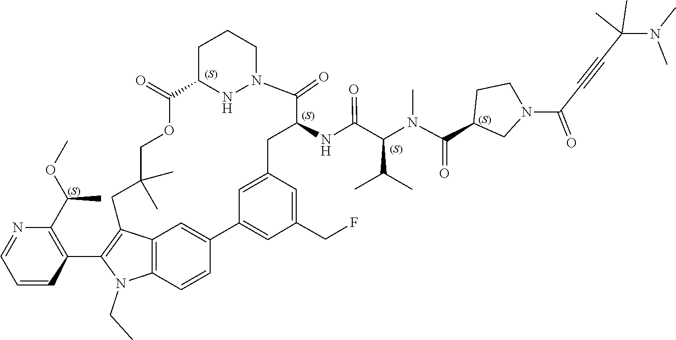 |
| BA659 | 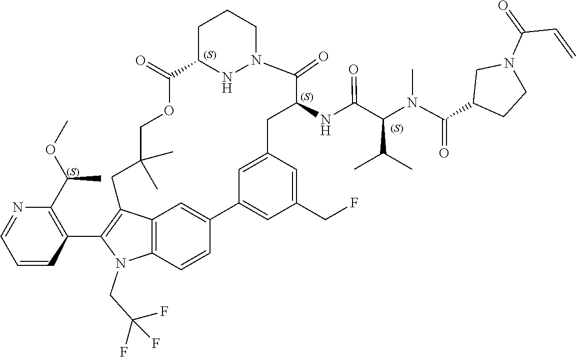 |
| BA660 | 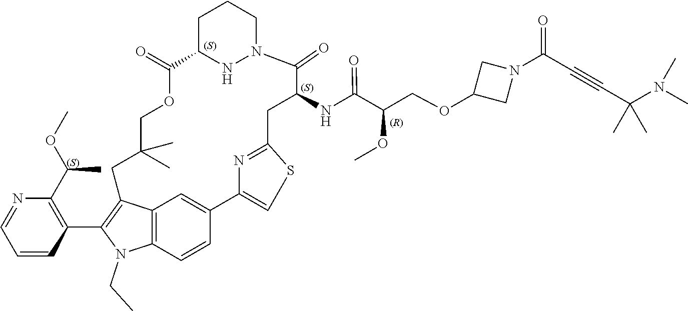 |
| BA661 | 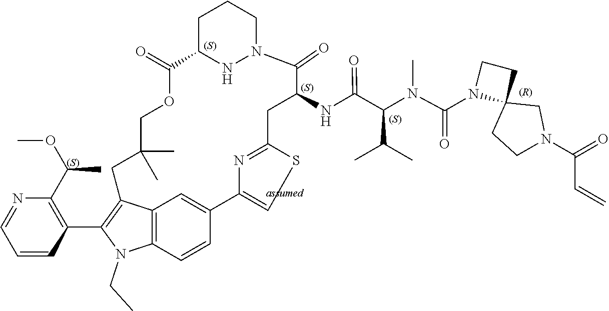 |
| BA662 | 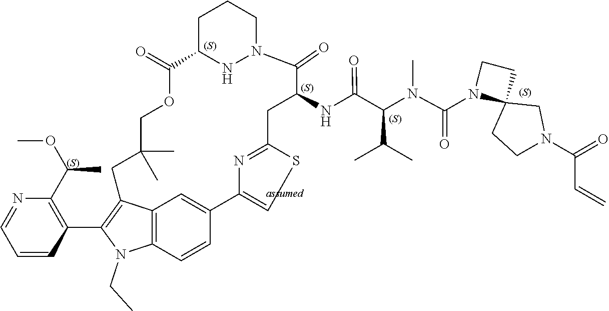 |
| BA663 | 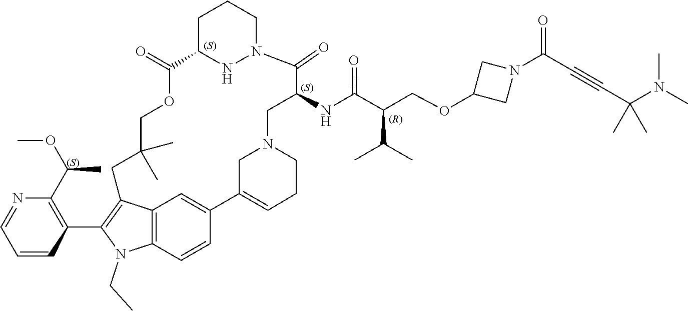 |
| BA664 | 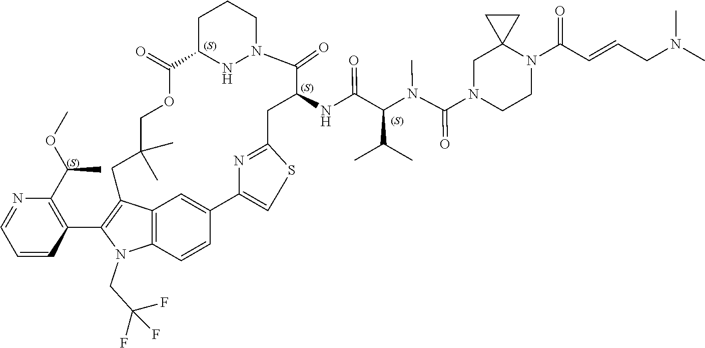 |
| BA665 | 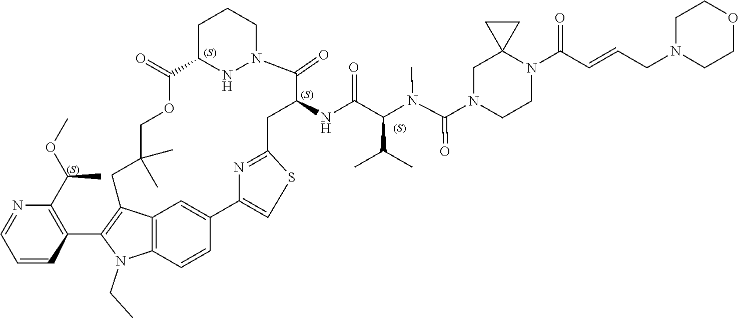 |
| BA666 | 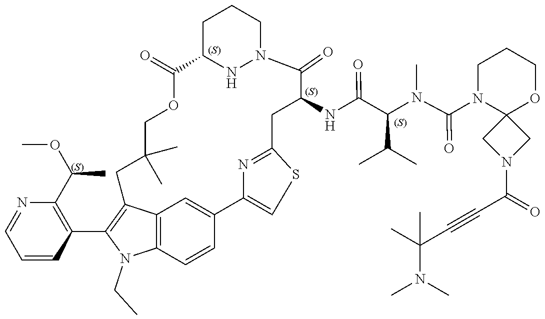 |
| BA667 | 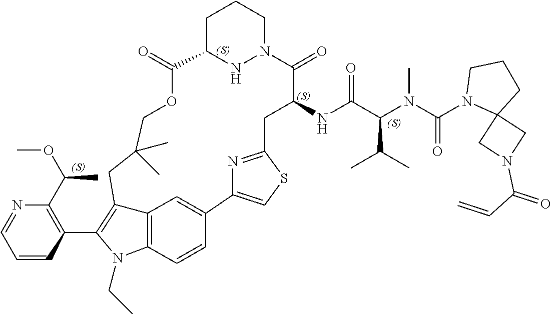 |
| BA668 | 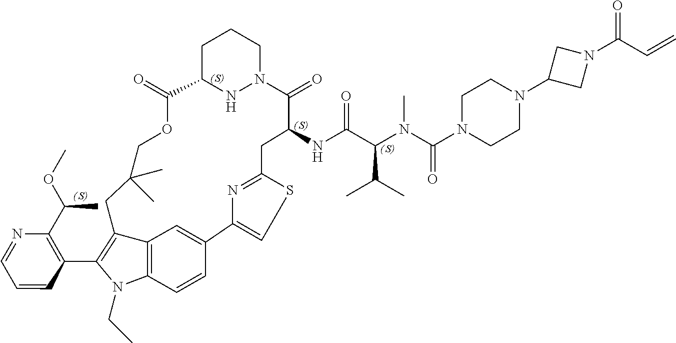 |
| BA669 | 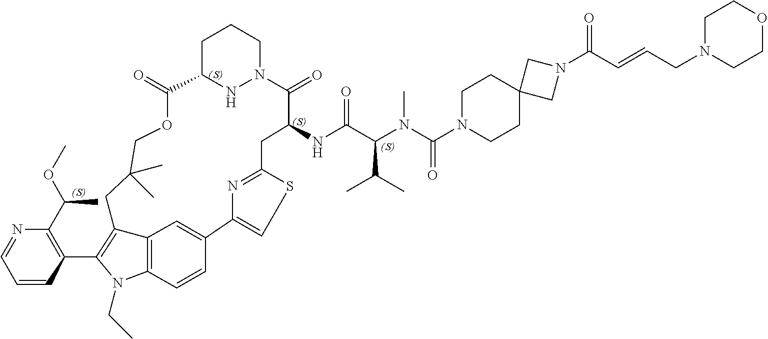 |
| BA670 | 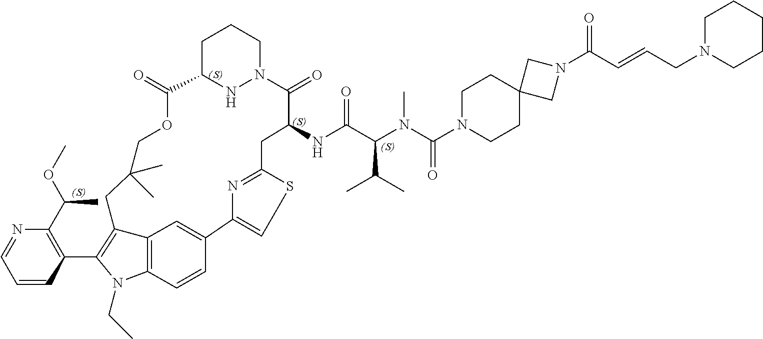 |
| BA671 | 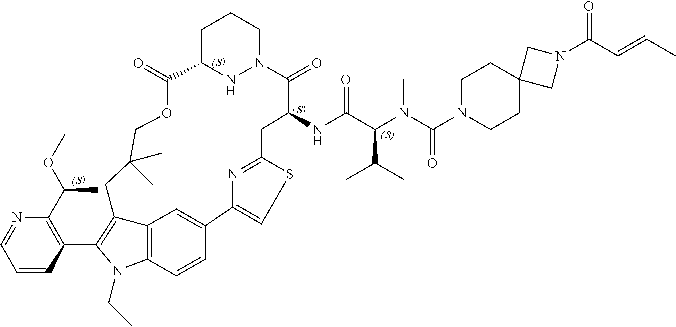 |
| BA672 | 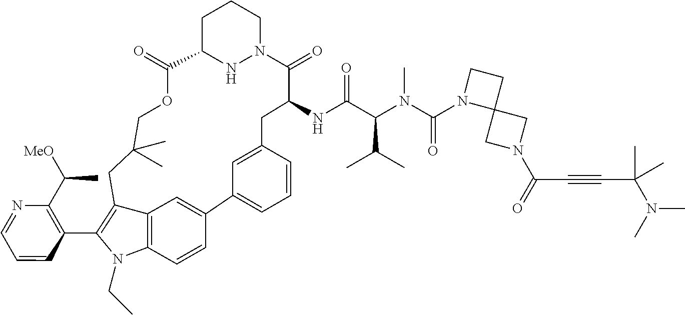 |
| BA673 | 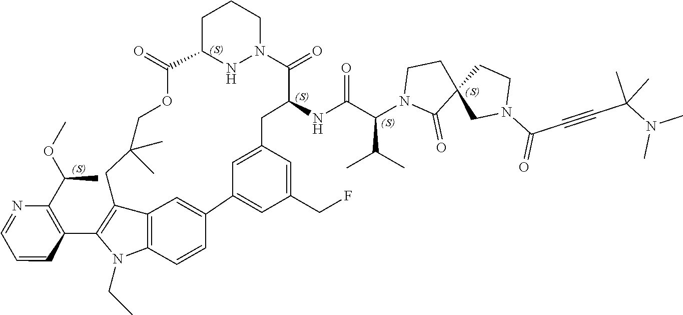 |
| BA674 | 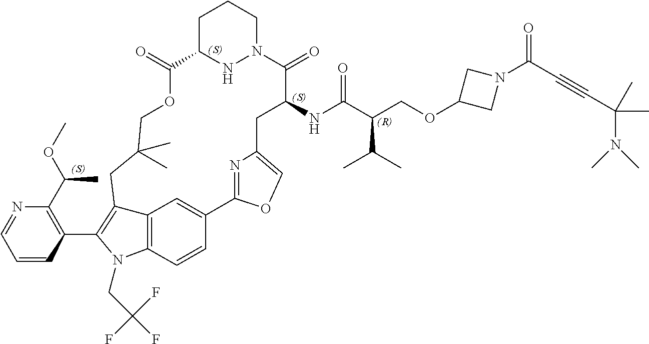 |
| BA675 | 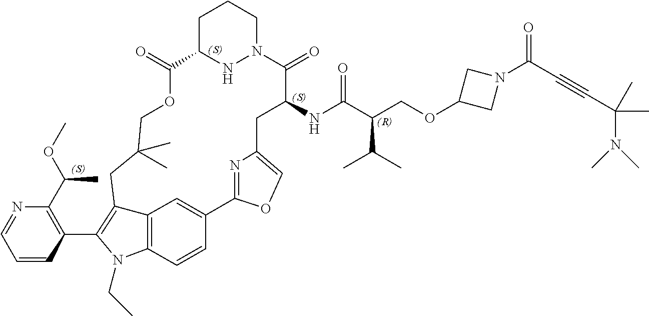 |
| BA676 | 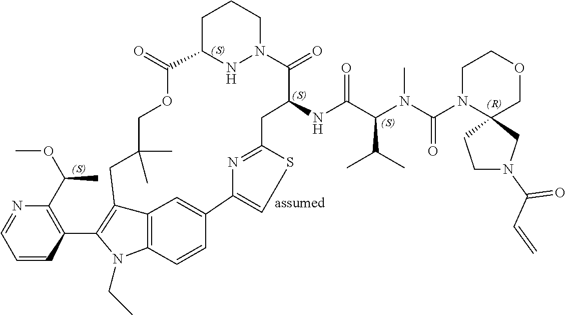 |
| BA677 | 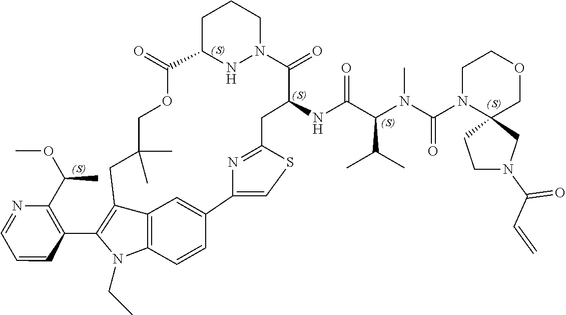 |
| BA678 | 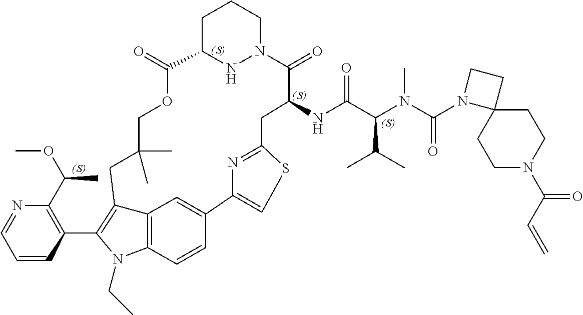 |
| BA679 | 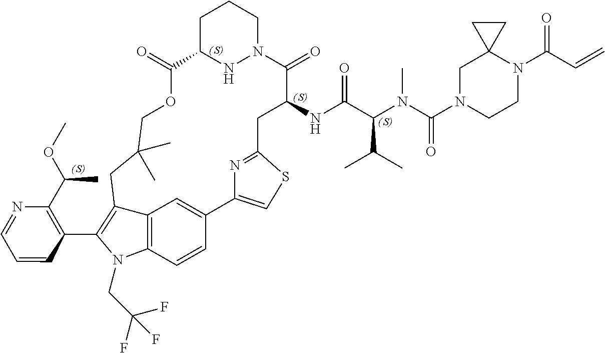 |
| BA680 | 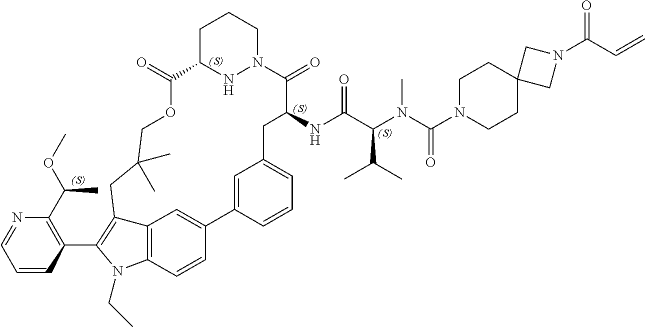 |
| BA681 | 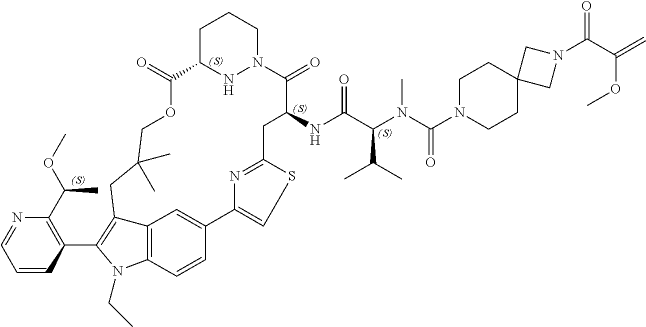 |
| BA682 | 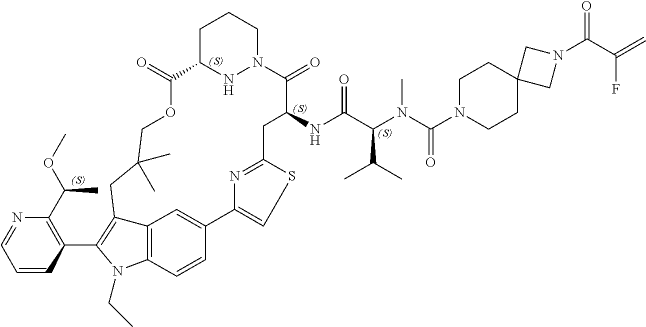 |
| BA683 | 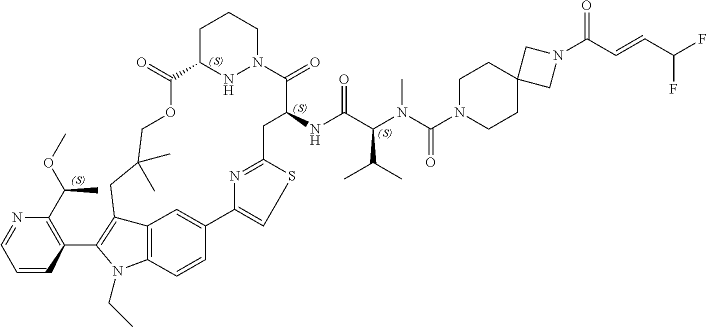 |
| BA684 | 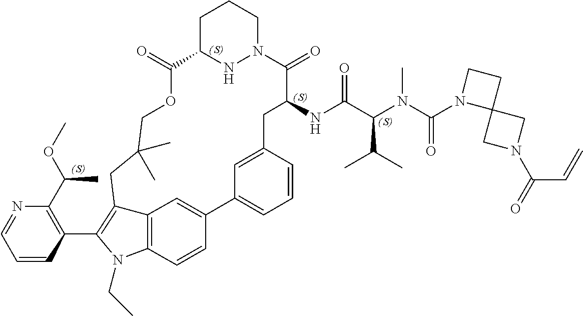 |
| BA685 | 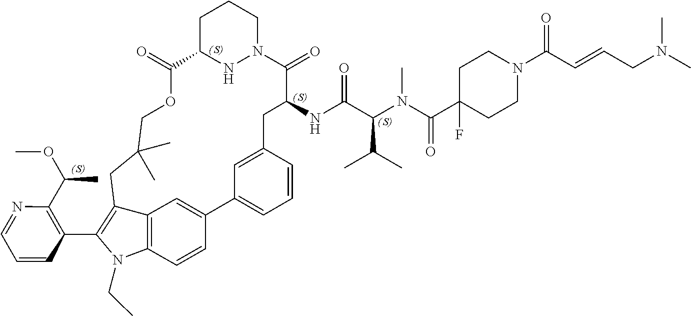 |
| BA686 | 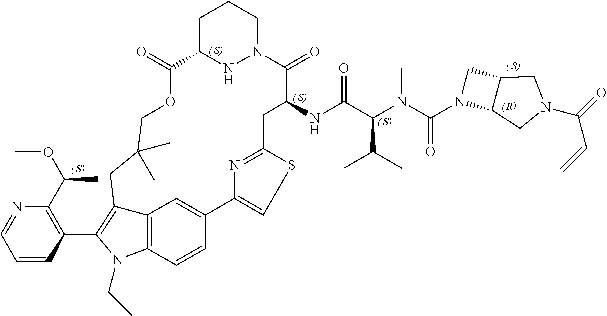 |
| BA687 | 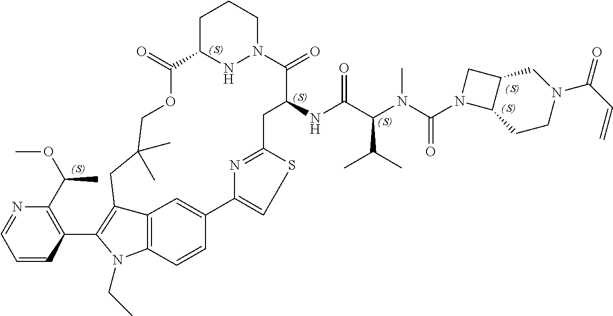 |
| BA688 | 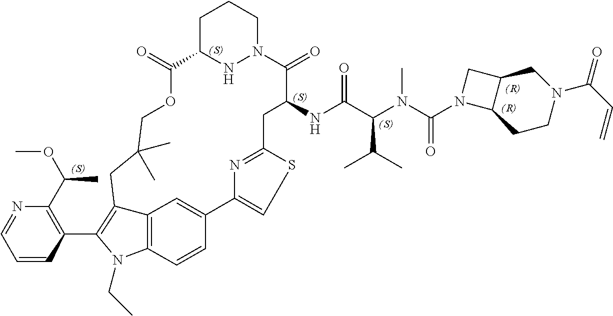 |
| BA689 | 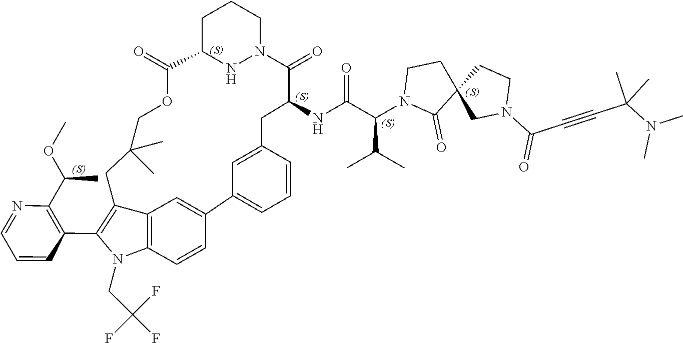 |
| BA690 | 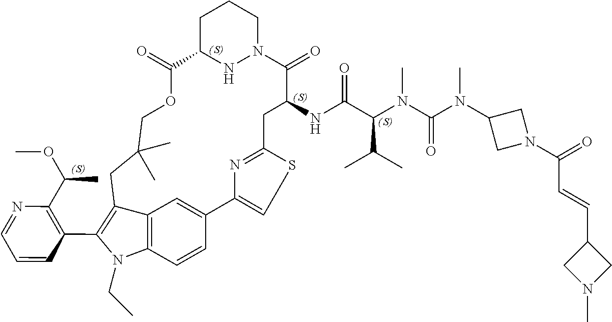 |
| BA691 | 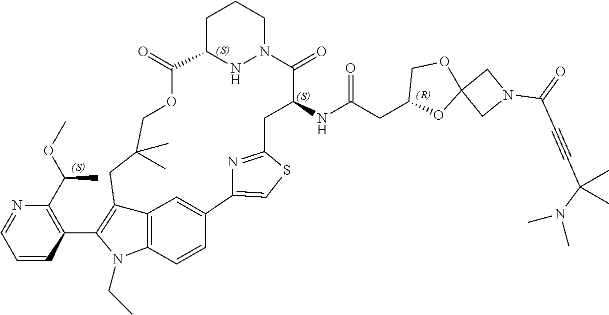 |
| BA692 | 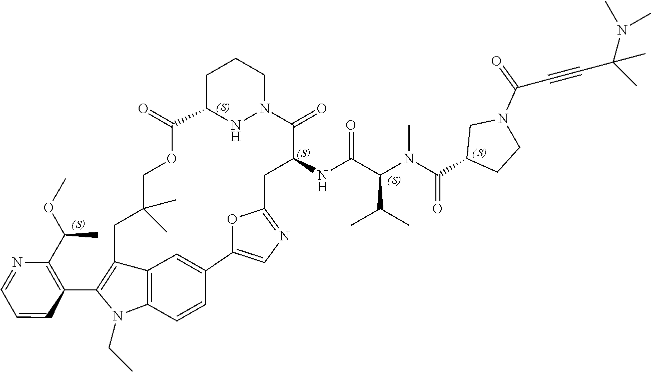 |
| BA693 | 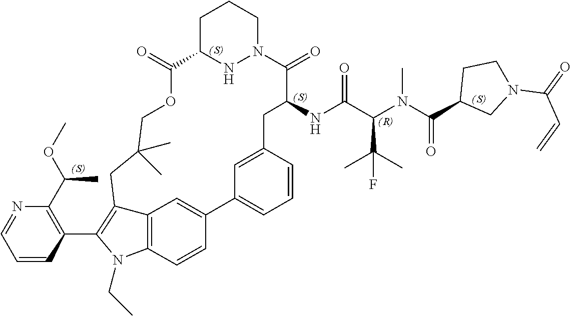 |
| BA694 | 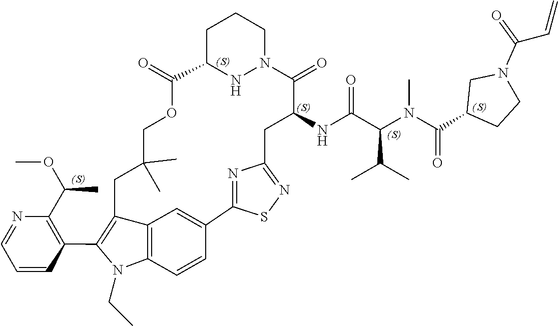 |
| BA695 | 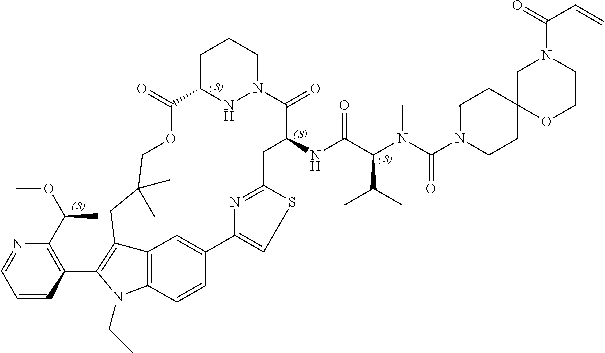 |
| BA696 | 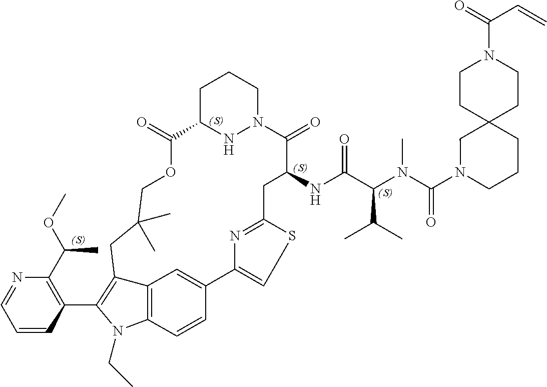 |
| BA697 | 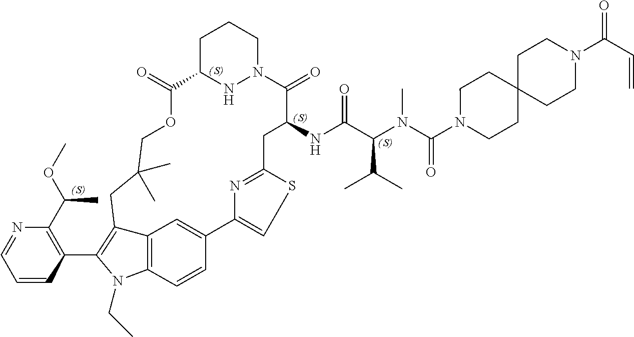 |
| BA698 | 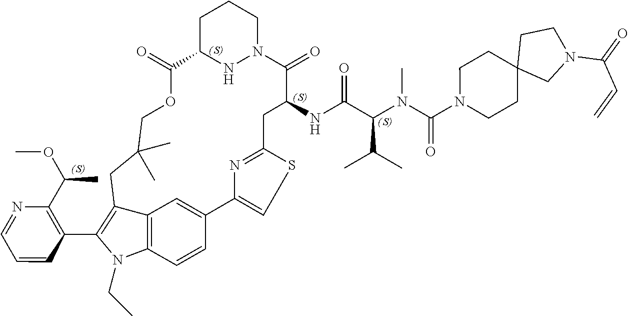 |
| BA699 | 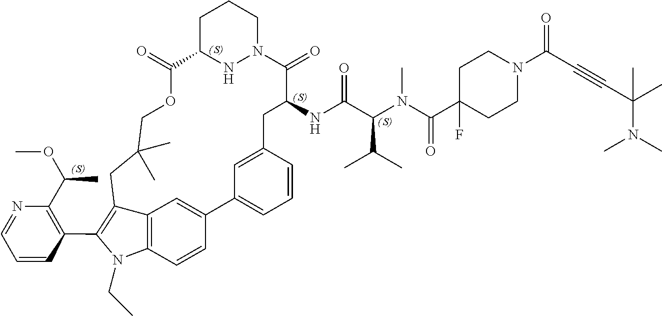 |
| BA700 | 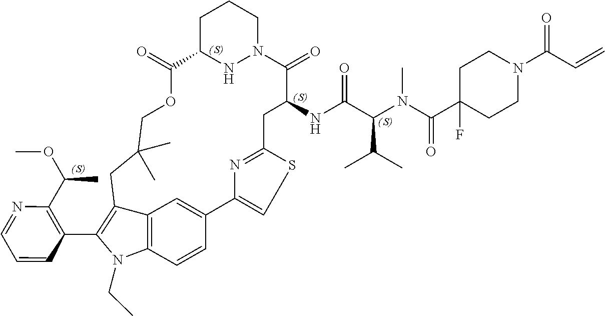 |
| BA701 | 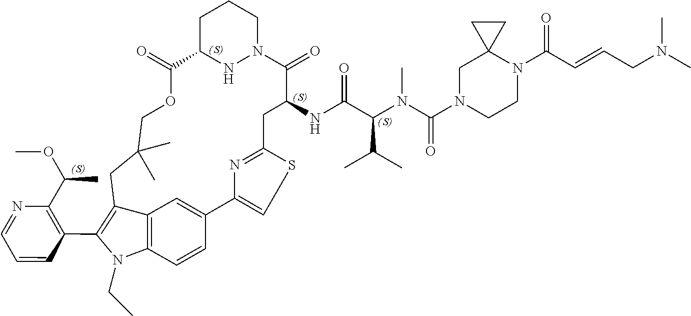 |
| BA702 | 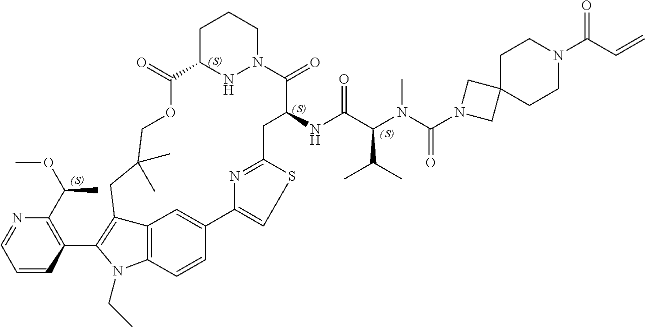 |
| BA703 | 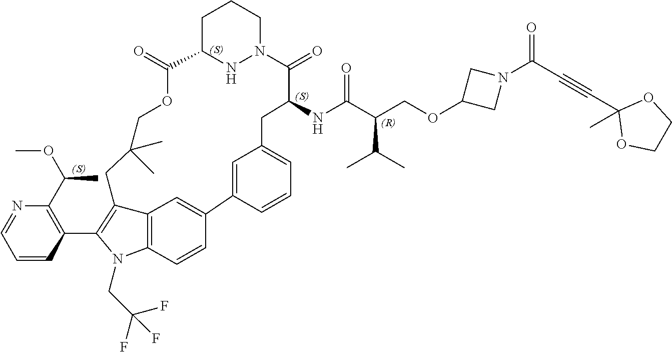 |
| BA704 | 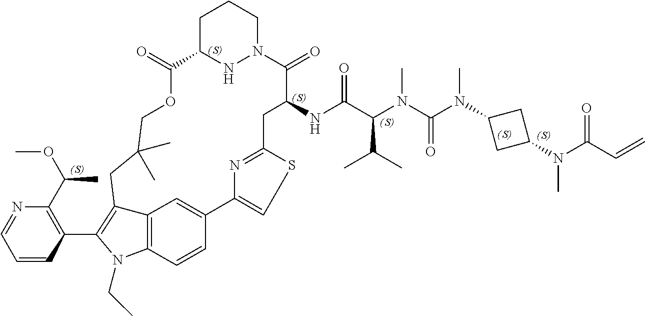 |
| BA705 | 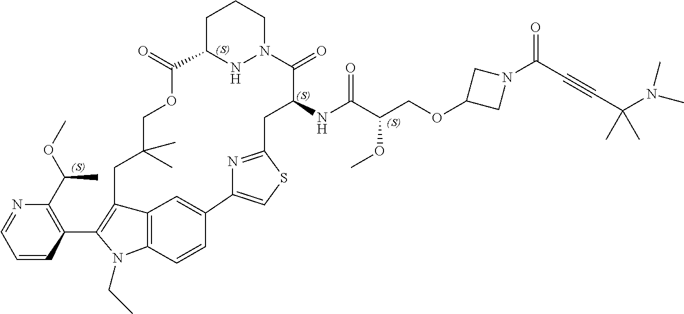 |
| BA706 | 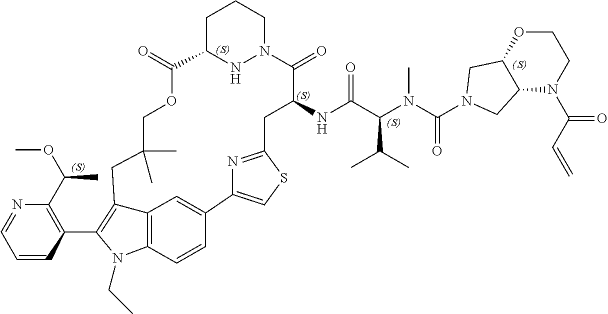 |
| BA707 | 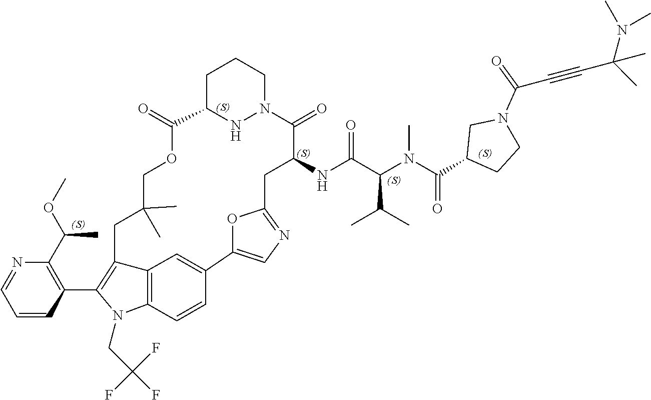 |
| BA708 | 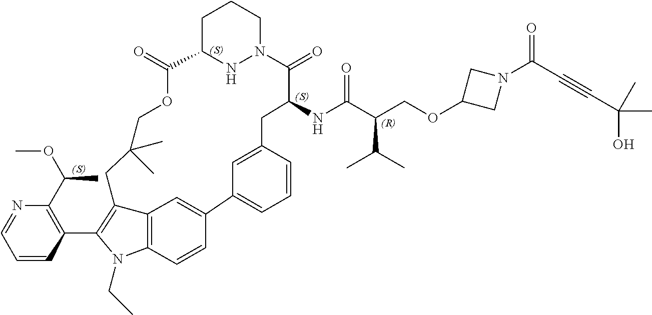 |
| BA709 | 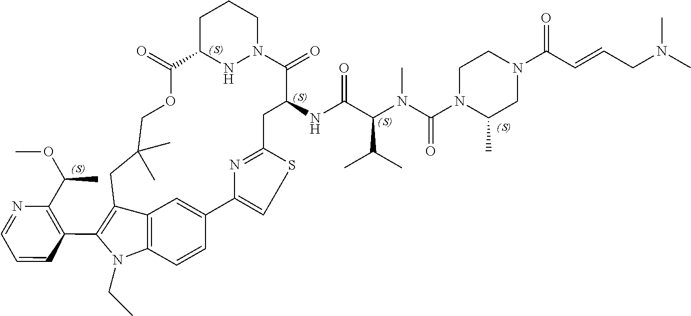 |
| BA710 | 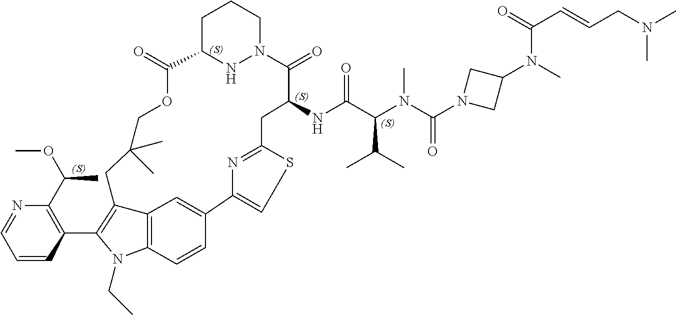 |
| BA711 | 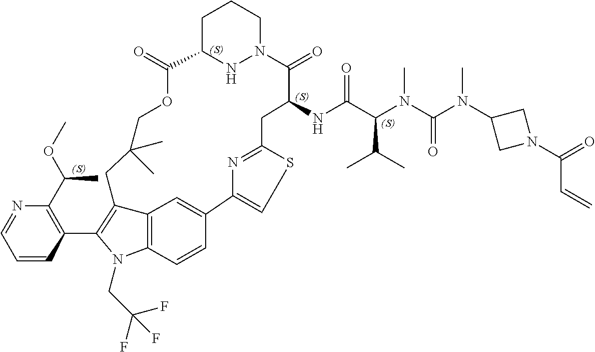 |
| BA712 | 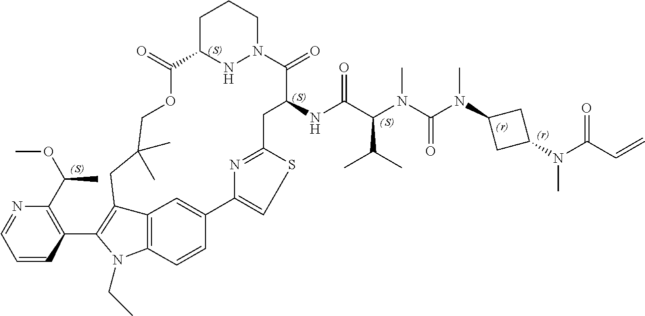 |
| BA713 | 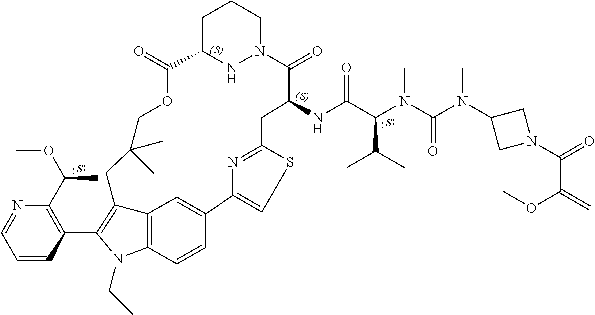 |
| BA714 | 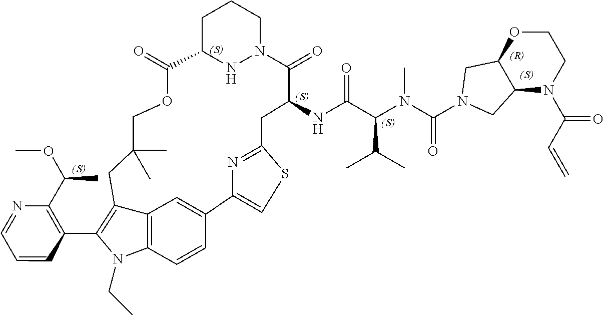 |
| BA715 | 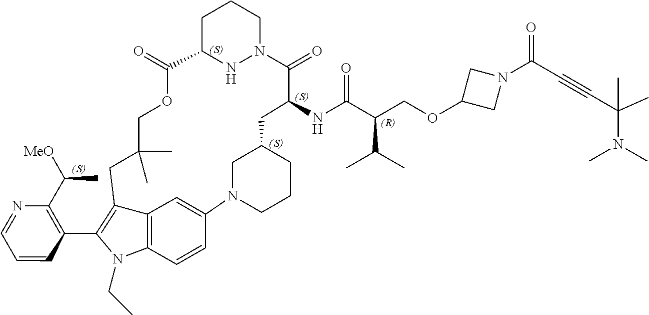 |
| BA716 | 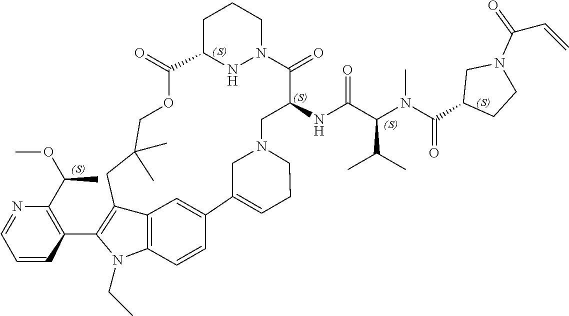 |
| BA717 | 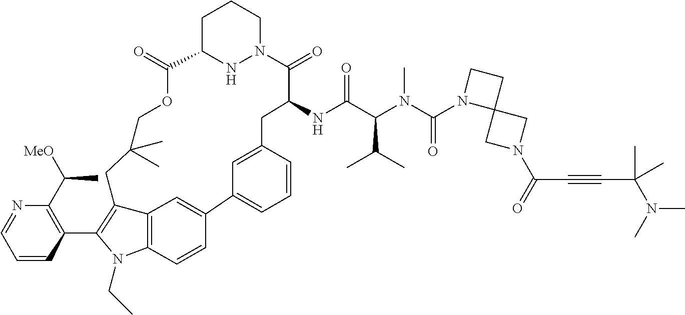 |
| BA718 | 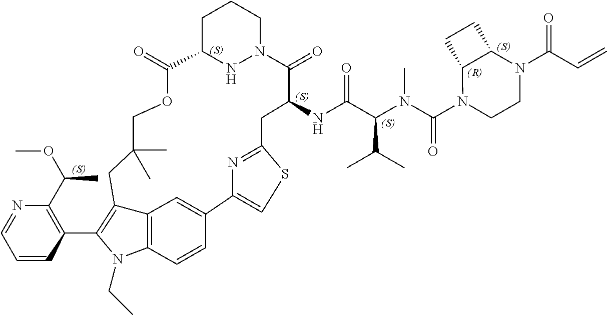 |
| BA719 | 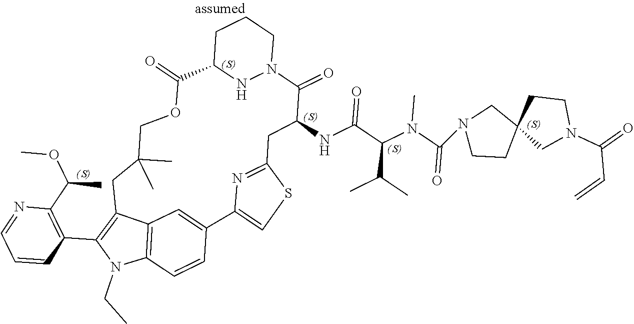 |
| BA720 | 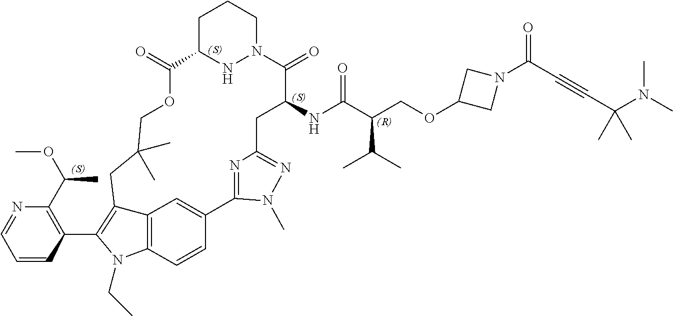 |
| BA721 | 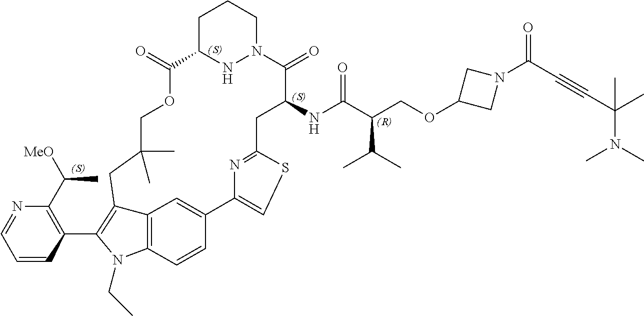 |
| BA722 | 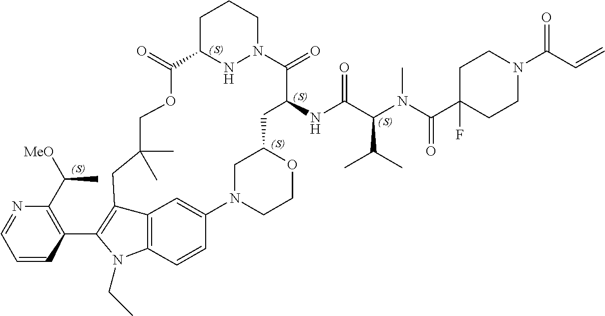 |
| BA723 | 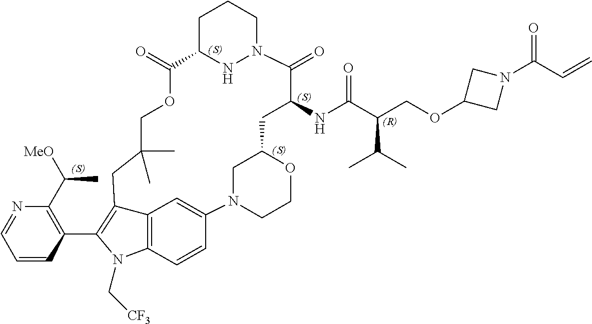 |
| BA724 | 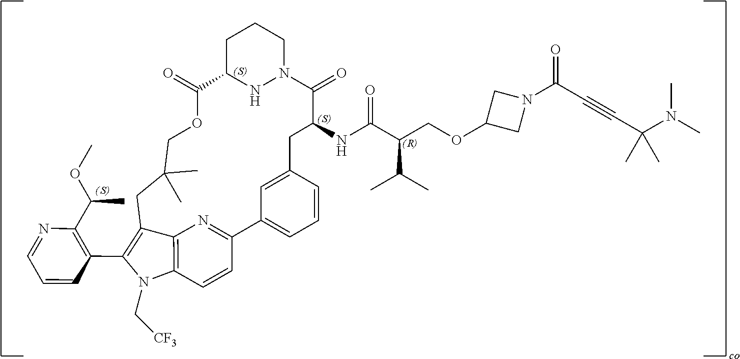 |
| BA725 | 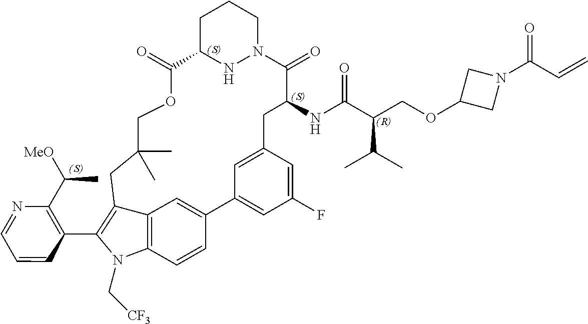 |
| BA726 | 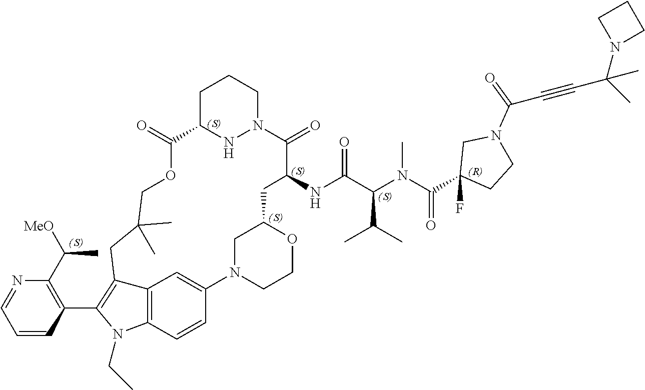 |
| BA727 | 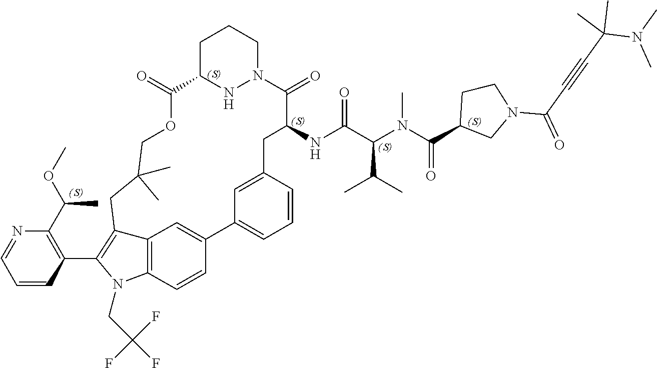 |
| BA728 | 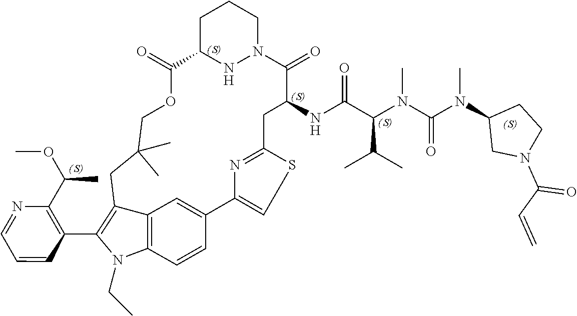 |
| BA729 | 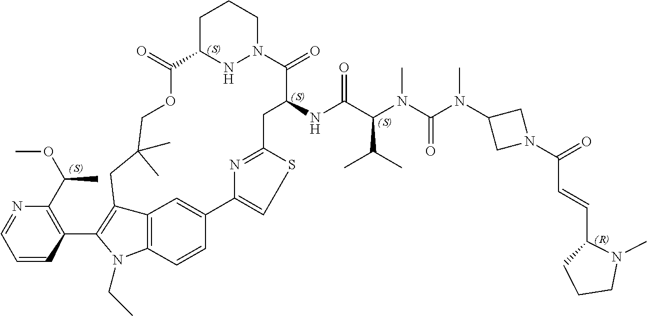 |
| BA730 | 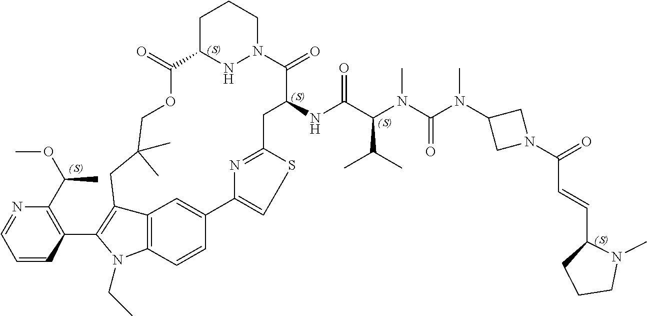 |
| BA731 | 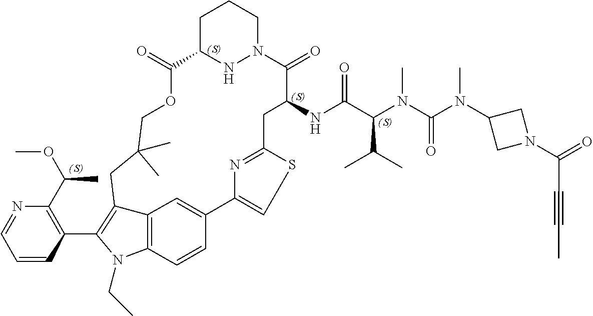 |
| BA732 | 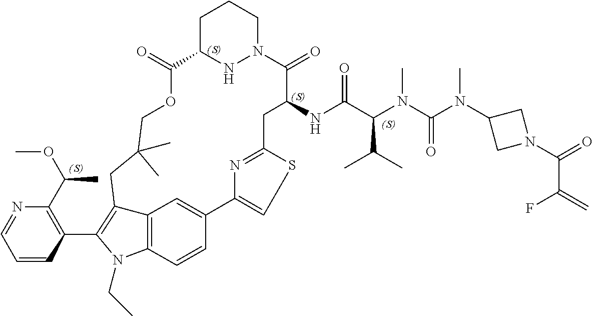 |
| BA733 | 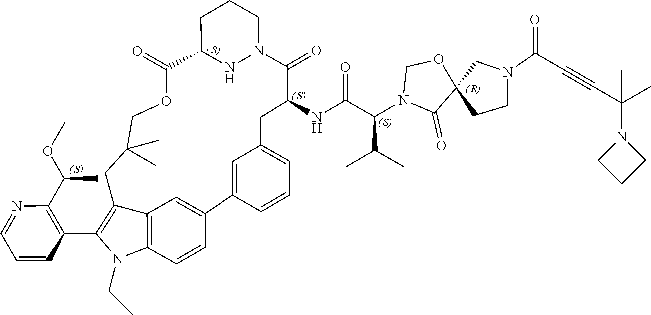 |
| BA734 | 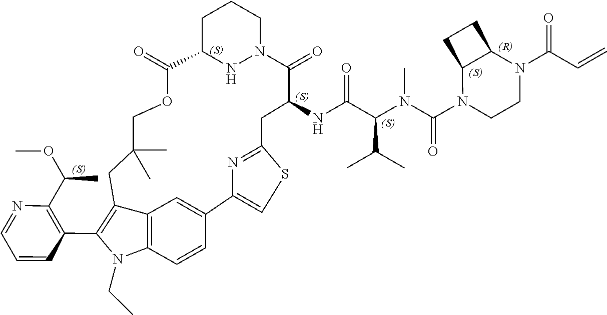 |
| BA735 | 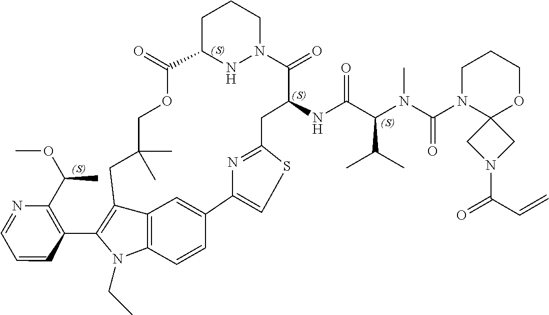 |
| BA736 | 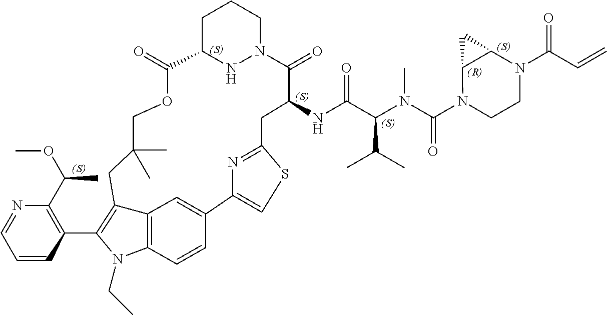 |
| BA737 | 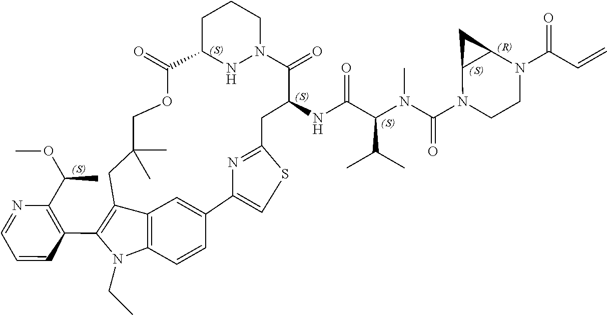 |
| BA738 | 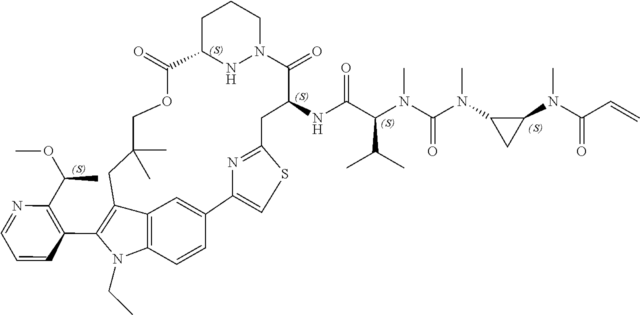 |
| BA739 | 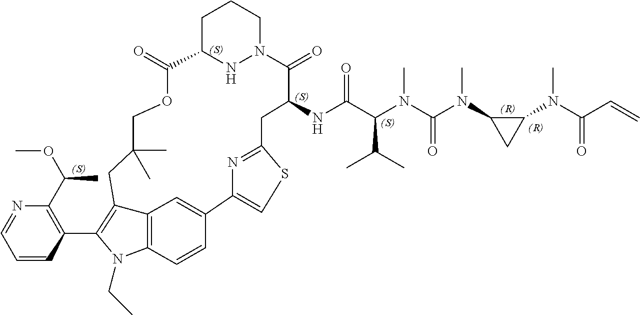 |
| BA740 | 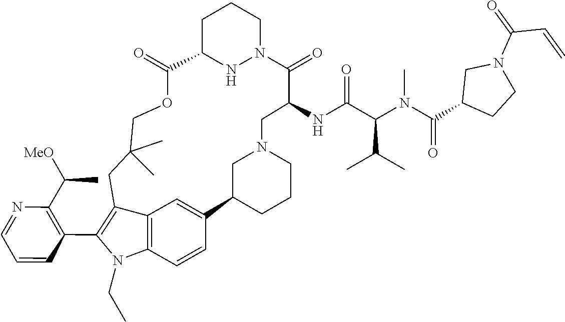 |
| BA741 | 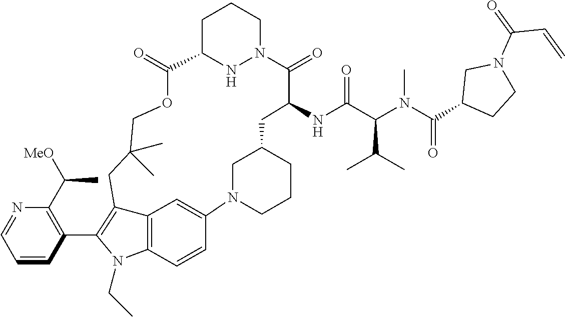 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particluar embodiments, an atropisomer of a compound of the foregoing table is contemplated.
Brackets are to be ignored.
*The activity of this stereoisomer may, in fact, be attributable to the presence of a small amount of the steroisomer with the (S) configuration at the —NC(O)—CH(CH₃)₂—N(CH₃)— position.

In some embodiments, a compound of Table B2 is provided, or a pharmaceutically acceptable salt thereof. In some embodiments, the RAS(ON) inhibitor is selected from Table B2, or a pharmaceutically acceptable salt or atropisomer thereof.

TABLE B2
Certain Compounds of the Present Invention

| Ex # | Structure |
|---|---|
| BB1 | 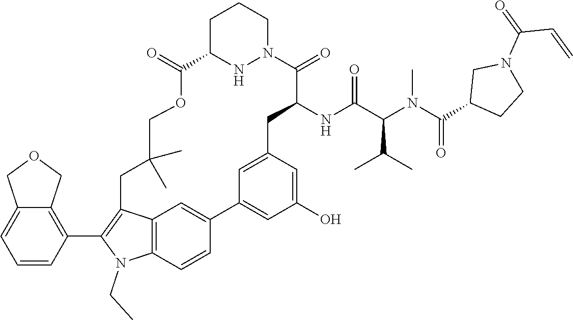 [SMILES: C=CC(=O)N1CC[C@H](C(=O)N(C)[C@H](C(=O)N[C@H]2Cc3cc(O)cc(c3)-c3ccc4c(c3)c(c(-c3cccc5c3COC5)n4CC)CC(C)(C)COC(=O)[C@@H]3CCCN(N3)C2=O)C(C)C)C1] |
| BB2 | 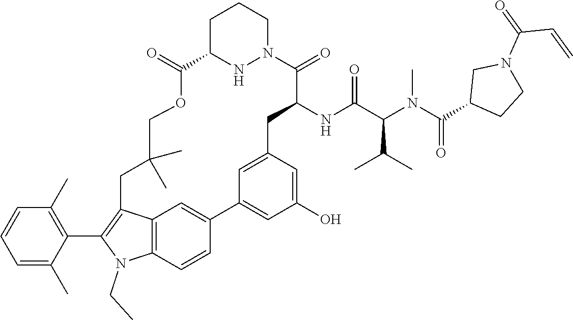 [SMILES: C=CC(=O)N1CC[C@H](C(=O)N(C)[C@H](C(=O)N[C@H]2Cc3cc(O)cc(c3)-c3ccc4c(c3)c(c(-c3c(C)cccc3C)n4CC)CC(C)(C)COC(=O)[C@@H]3CCCN(N3)C2=O)C(C)C)C1] |
| BB3 | 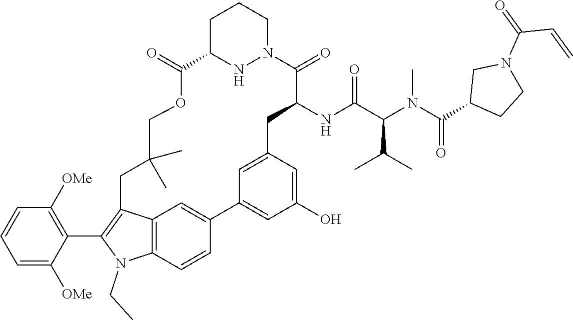 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cccc3OC)n4CC)CC(C)(C)COC(=O)[C@@H]3CCCN(N3)C2=O)C(C)C)C1] |
| BB4 | 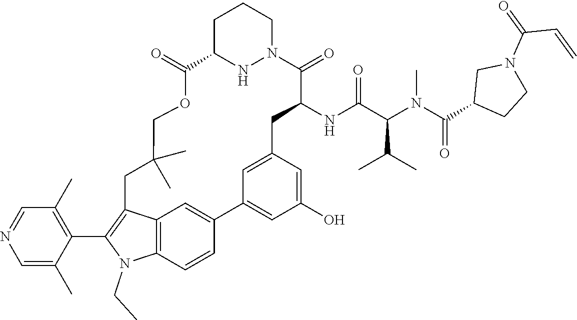 [SMILES: C=CC(=O)N1CC[C@H](C(=O)N(C)[C@H](C(=O)N[C@H]2Cc3cc(O)cc(c3)-c3ccc4c(c3)c(c(-c3c(C)cncc3C)n4CC)CC(C)(C)COC(=O)[C@@H]3CCCN(N3)C2=O)C(C)C)C1] |
| BB5 | 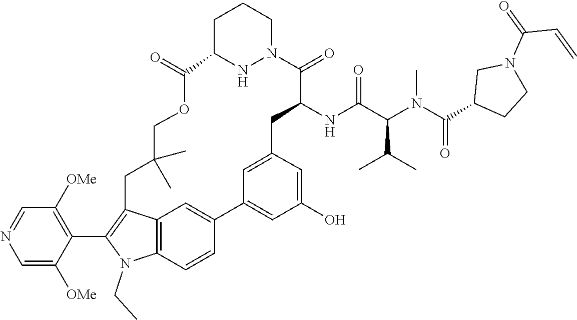 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cncc3OC)n4CC)CC(C)(C)COC(=O)[C@@H]3CCCN(N3)C2=O)C(C)C)C1] |
| BB6 | 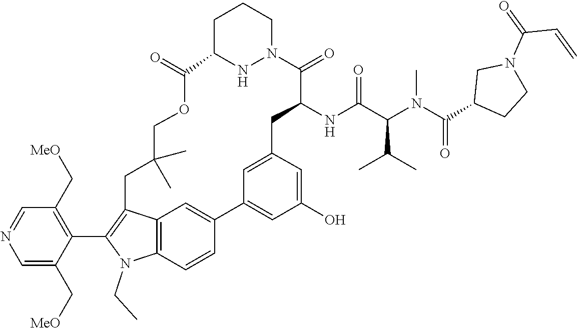 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cncc3COC)n4CC)CC(C)(C)COC(=O)[C@@H]3CCCN(N3)C2=O)C(C)C)C1] |
| BB7 | 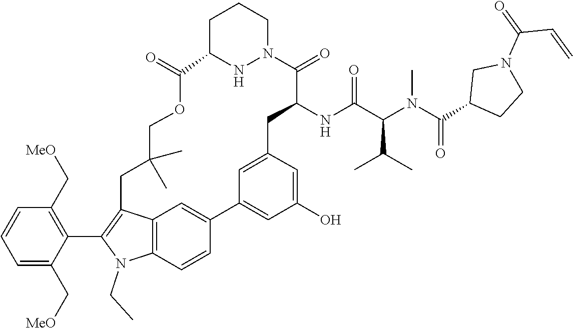 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cccc3COC)n4CC)CC(C)(C)COC(=O)[C@@H]3CCCN(N3)C2=O)C(C)C)C1] |
| BB11 | 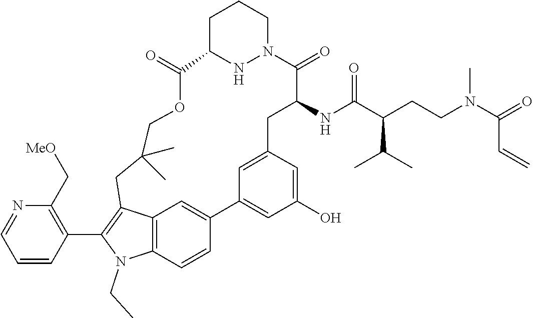 [SMILES: C=CC(=O)N(C)CC[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| BB12 | 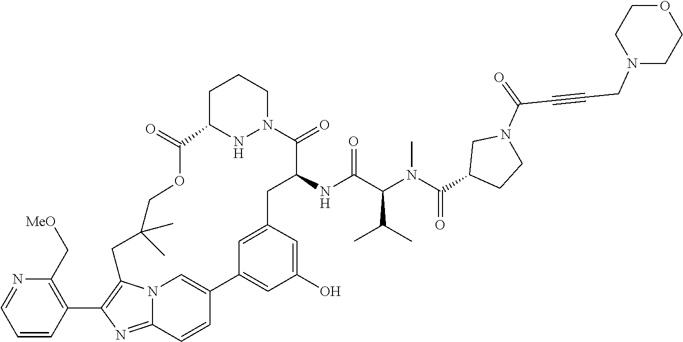 [SMILES: COCc1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CCN2CCOCC2)C1)Cc1cc(O)cc-3c1] |
| BB13 | 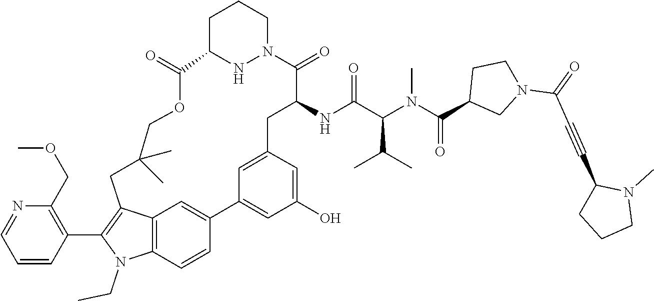 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BB18 | 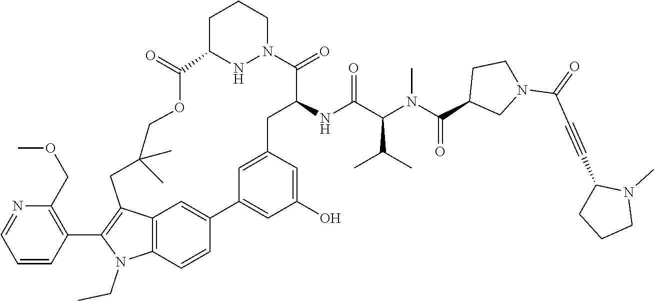 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H]3CCCN3C)C1)C(=O)N1CCC[C@H](N1)C(=O)OCC(C)(C)C2] |
| BB21 | 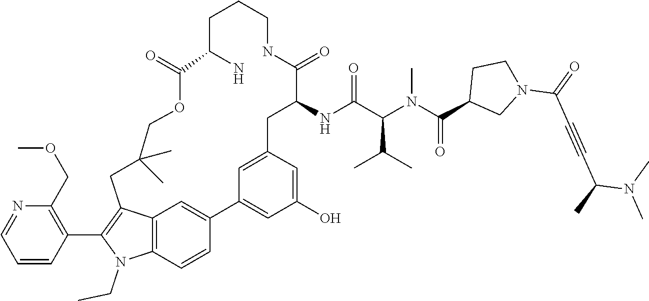 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H](C)N(C)C)C1)C(=O)NCCC[C@H](N)C(=O)OCC(C)(C)C2] |
| BB22 | 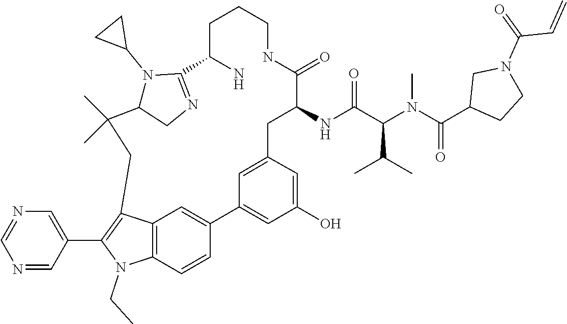 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)C3CN=C([C@@H](N)CCCNC2=O)N3C2CC2)C(C)C)C1] |
| BB25 | 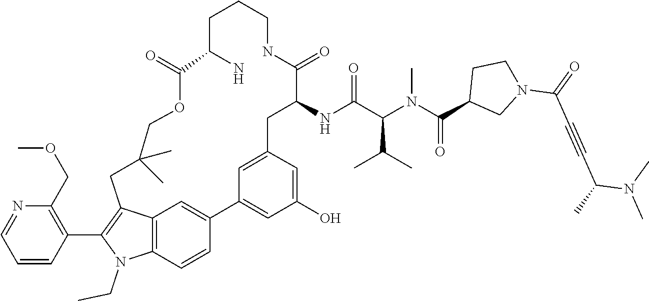 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H](C)N(C)C)C1)C(=O)NCCC[C@H](N)C(=O)OCC(C)(C)C2] |
| BB27 | 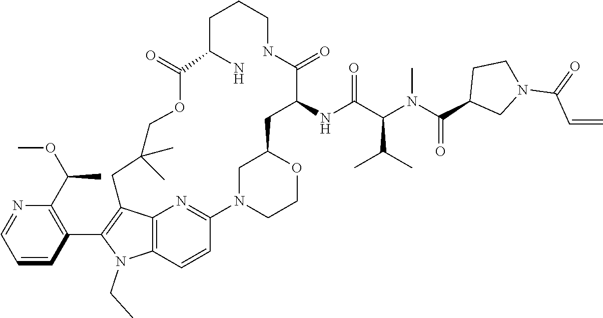 |
| BB28 | 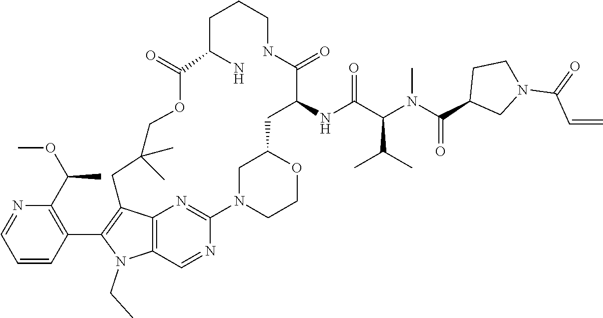 [SMILES: C=CC(=O)N1CC[C@H](C(=O)N(C)[C@H](C(=O)N[C@H]2C[C@H]3CN(CCO3)c3ncc4c(n3)c(c(-c3cccnc3[C@H](C)OC)n4CC)CC(C)(C)COC(=O)[C@@H](N)CCCNC2=O)C(C)C)C1] |
| BB29 | 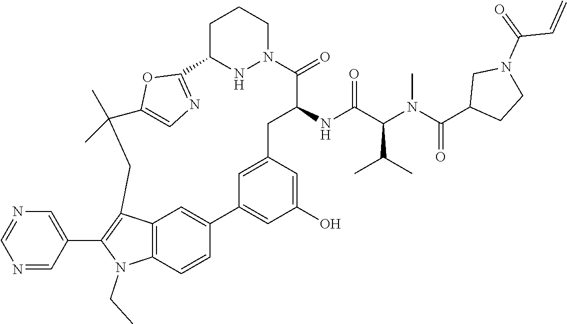 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)C3=CN=C(O3)[C@@H]3CCCN(N3)C2=O)C(C)C)C1] |
| BB30 | 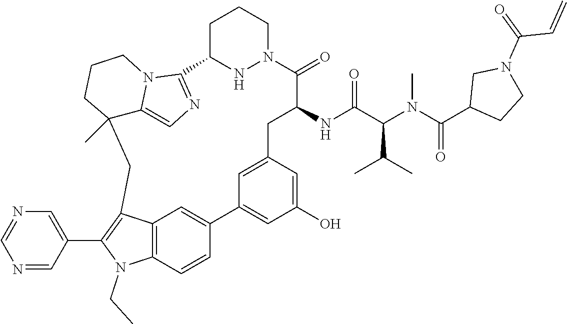 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC3(C)CCCn4c3cnc4[C@@H]3CCCN(N3)C2=O)C(C)C)C1] |
| BB32 | 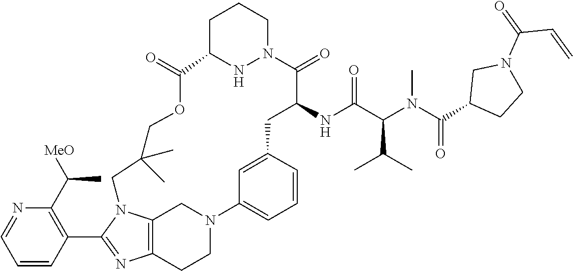 |
| BB34 | 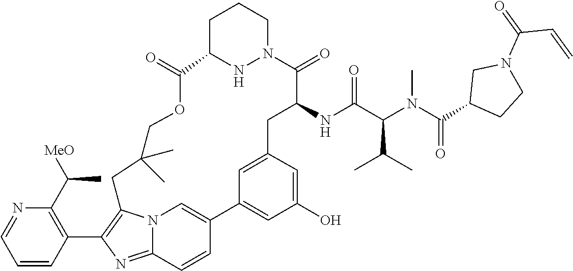 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)c(n4c3)CC(C)(C)COC(=O)[C@@H]3CCCN(N3)C2=O)C(C)C)C1] |
| BB38 | 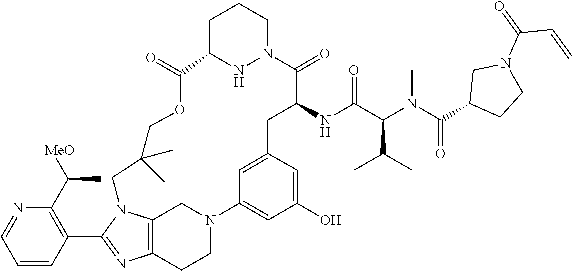 [SMILES: C=CC(=O)N1CC[C@H](C(=O)N(C)[C@H](C(=O)N[C@H]2Cc3cc(O)cc(c3)N3CCc4nc(-c5cccnc5[C@H](C)OC)n(c4C3)CC(C)(C)COC(=O)[C@@H]3CCCN(N3)C2=O)C(C)C)C1] |
| BB47 | 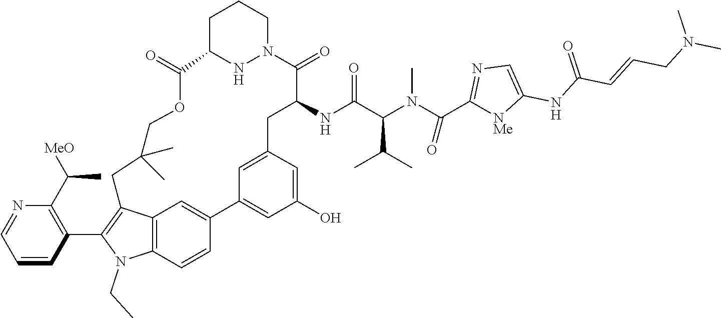 |
| BB64 | 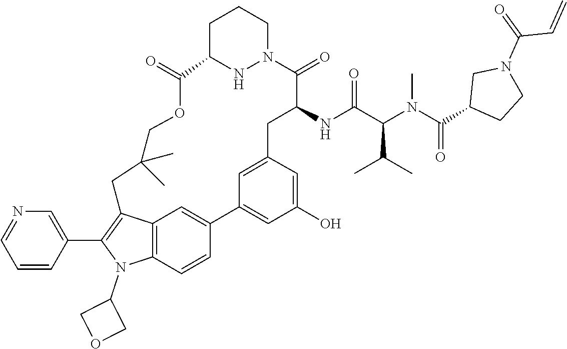 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C3COC3)CC(C)(C)COC(=O)[C@@H]3CCCN(N3)C2=O)C(C)C)C1] |
| BB65 | 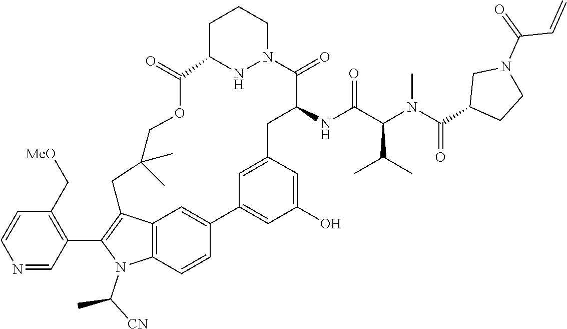 [SMILES: C=CC(=O)N1CC[C@H](C(=O)N(C)[C@H](C(=O)N[C@H]2Cc3cc(O)cc(c3)-c3ccc4c(c3)c(c(-c3cnccc3COC)n4[C@H](C)C#N)CC(C)(C)COC(=O)[C@@H]3CCCN(N3)C2=O)C(C)C)C1] |
| BB66 | 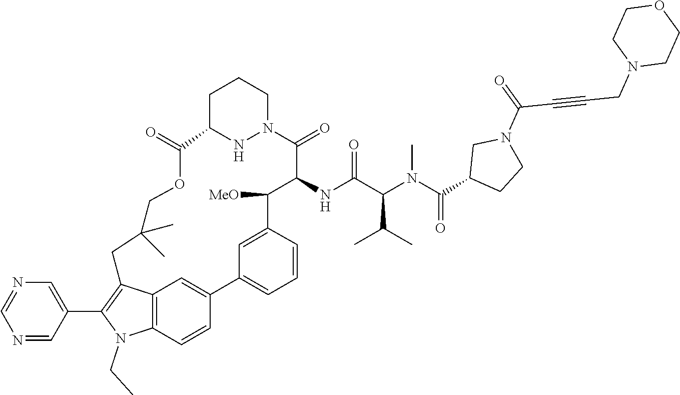 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB70 | 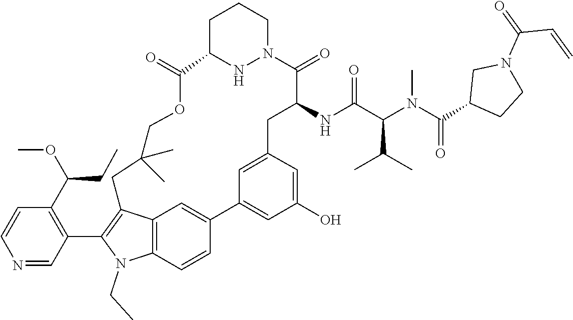 [SMILES: C=CC(=O)N1CC[C@H](C(=O)N(C)[C@H](C(=O)N[C@H]2Cc3cc(O)cc(c3)-c3ccc4c(c3)c(c(-c3cnccc3[C@H](CC)OC)n4CC)CC(C)(C)COC(=O)[C@@H]3CCCN(N3)C2=O)C(C)C)C1] |
| BB73 | 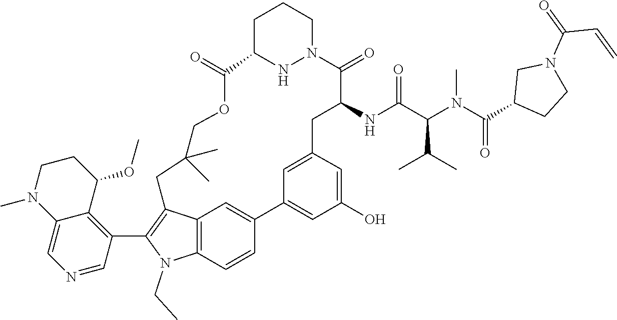 [SMILES: C=CC(=O)N1CC[C@H](C(=O)N(C)[C@H](C(=O)N[C@H]2Cc3cc(O)cc(c3)-c3ccc4c(c3)c(c(-c3cncc5c3[C@@H](OC)CCN5C)n4CC)CC(C)(C)COC(=O)[C@@H]3CCCN(N3)C2=O)C(C)C)C1] |
| BB74 | 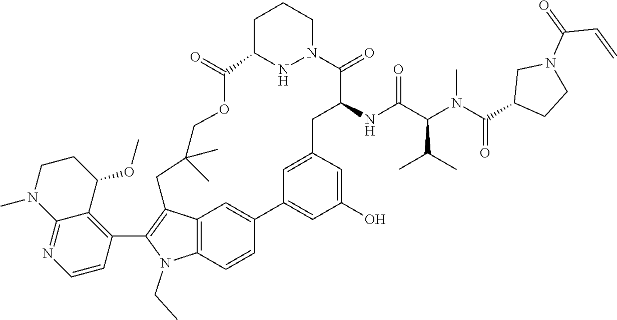 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CCN5C)n4CC)CC(C)(C)COC(=O)[C@@H]3CCCN(N3)C2=O)C(C)C)C1] |
| BB75 | 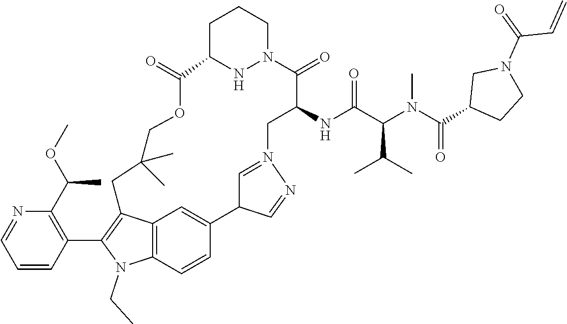 |
| BB76 | 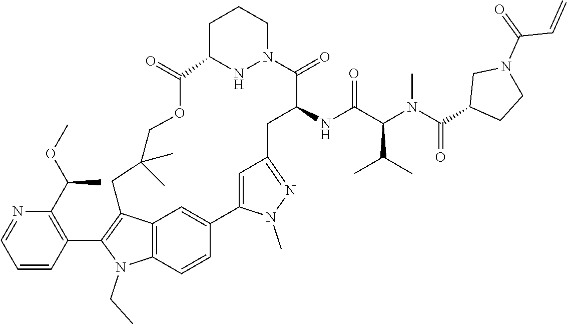 [SMILES: C=CC(=O)N1CC[C@H](C(=O)N(C)[C@H](C(=O)N[C@H]2Cc3cc(n(C)n3)-c3ccc4c(c3)c(c(-c3cccnc3[C@H](C)OC)n4CC)CC(C)(C)COC(=O)[C@@H]3CCCN(N3)C2=O)C(C)C)C1] |
| BB77 | 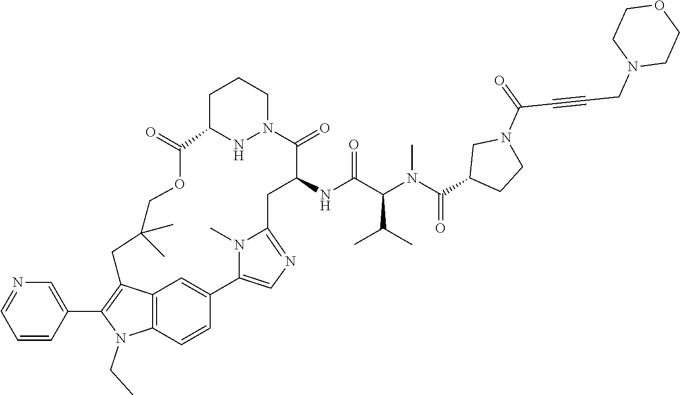 [SMILES: CCn1c(-c2cccnc2)c2c3cc(ccc31)-c1cnc(n1C)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB81 | 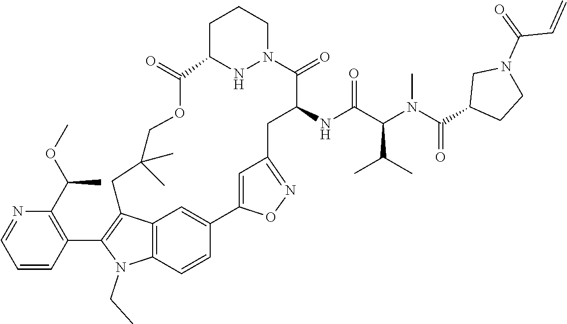 [SMILES: C=CC(=O)N1CC[C@H](C(=O)N(C)[C@H](C(=O)N[C@H]2Cc3cc(on3)-c3ccc4c(c3)c(c(-c3cccnc3[C@H](C)OC)n4CC)CC(C)(C)COC(=O)[C@@H]3CCCN(N3)C2=O)C(C)C)C1] |
| BB83 | 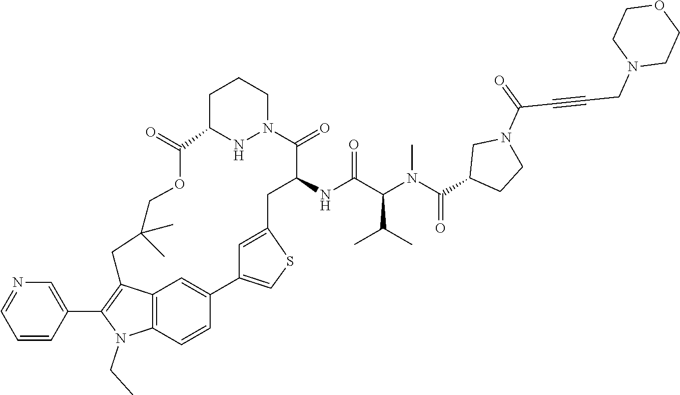 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB85 | 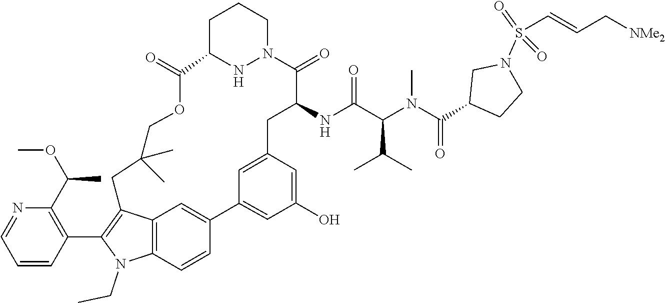 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)/C=C/CN(C)C)C1)C(=O)N1CCC[C@H](N1)C(=O)OCC(C)(C)C2] |
| BB86 | 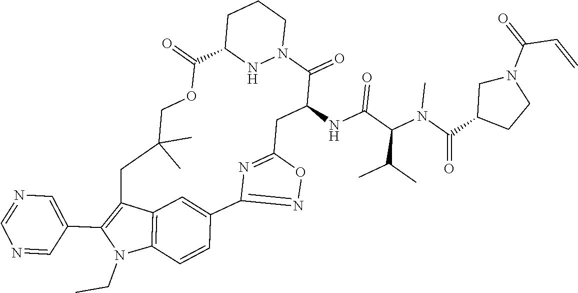 [SMILES: C=CC(=O)N1CC[C@H](C(=O)N(C)[C@H](C(=O)N[C@H]2Cc3nc(no3)-c3ccc4c(c3)c(c(-c3cncnc3)n4CC)CC(C)(C)COC(=O)[C@@H]3CCCN(N3)C2=O)C(C)C)C1] |
| BB87 | 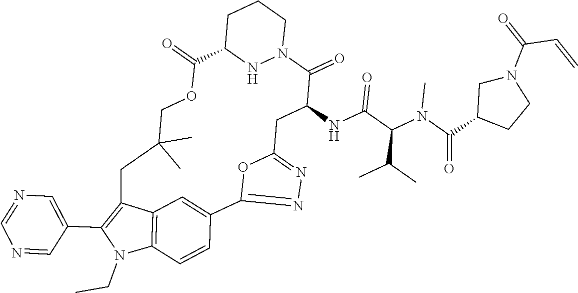 [SMILES: C=CC(=O)N1CC[C@H](C(=O)N(C)[C@H](C(=O)N[C@H]2CC3=NN=C(O3)c3ccc4c(c3)c(c(-c3cncnc3)n4CC)CC(C)(C)COC(=O)[C@@H]3CCCN(N3)C2=O)C(C)C)C1] |
| BB88 | 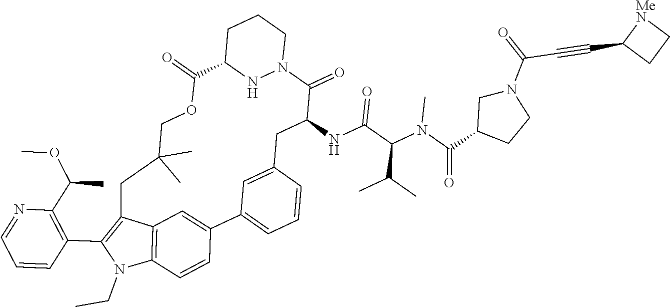 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB89 | 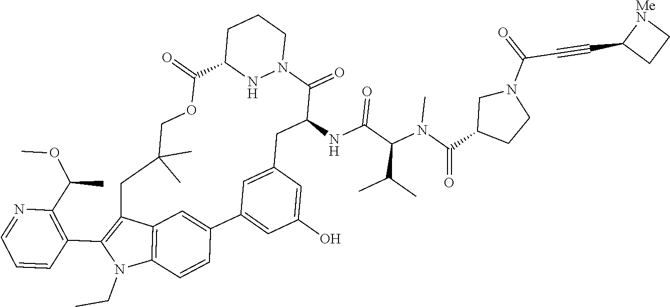 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB90 | 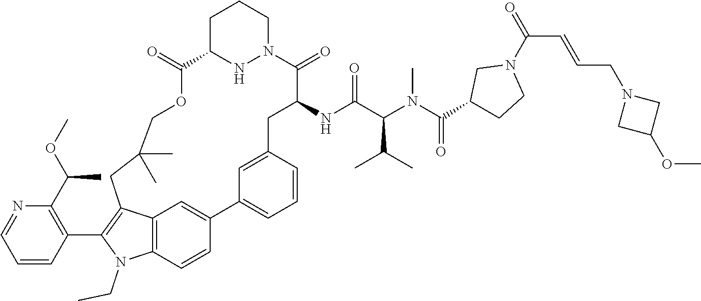 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)/C=C/CN3CC(OC)C3)C1)C(=O)N1CCC[C@H](N1)C(=O)OCC(C)(C)C2] |
| BB91 | 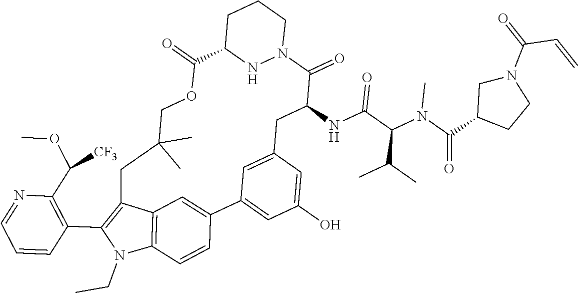 [SMILES: C=CC(=O)N1CC[C@H](C(=O)N(C)[C@H](C(=O)N[C@H]2Cc3cc(O)cc(c3)-c3ccc4c(c3)c(c(-c3cccnc3[C@@H](OC)C(F)(F)F)n4CC)CC(C)(C)COC(=O)[C@@H]3CCCN(N3)C2=O)C(C)C)C1] |
| BB96 | 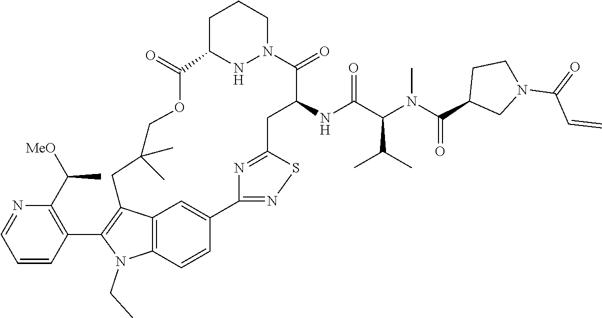 [SMILES: C=CC(=O)N1CC[C@H](C(=O)N(C)[C@H](C(=O)N[C@H]2Cc3nc(ns3)-c3ccc4c(c3)c(c(-c3cccnc3[C@H](C)OC)n4CC)CC(C)(C)COC(=O)[C@@H]3CCCN(N3)C2=O)C(C)C)C1] |
| BB97 | 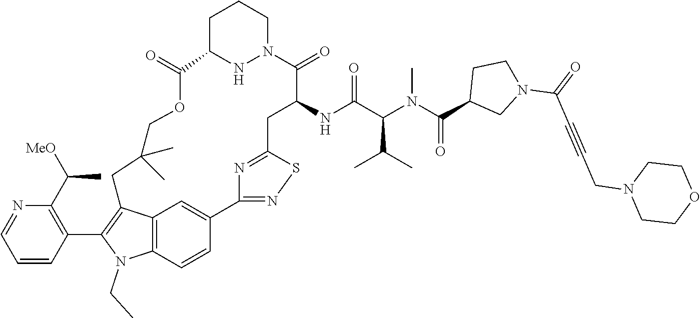 [SMILES: CCn1c(-c2cccnc2[C@H](C)OC)c2c3cc(ccc31)-c1nsc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB102 | 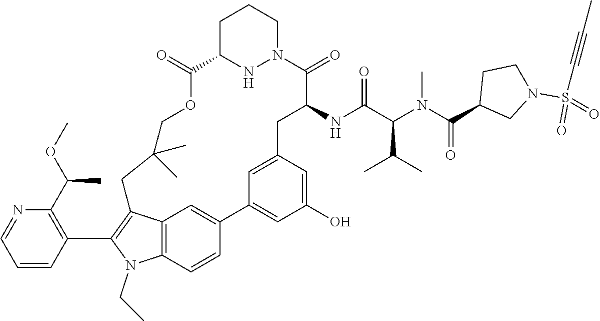 [SMILES: CC#CS(=O)(=O)N1CC[C@H](C(=O)N(C)[C@H](C(=O)N[C@H]2Cc3cc(O)cc(c3)-c3ccc4c(c3)c(c(-c3cccnc3[C@H](C)OC)n4CC)CC(C)(C)COC(=O)[C@@H]3CCCN(N3)C2=O)C(C)C)C1] |
| BB103 | 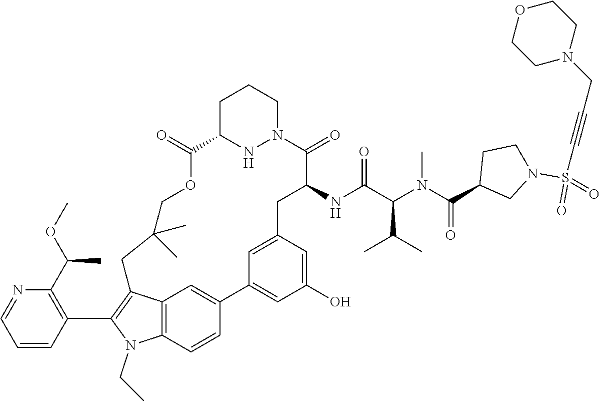 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)C#CCN3CCOCC3)C1)C(=O)N1CCC[C@H](N1)C(=O)OCC(C)(C)C2] |
| BB104 | 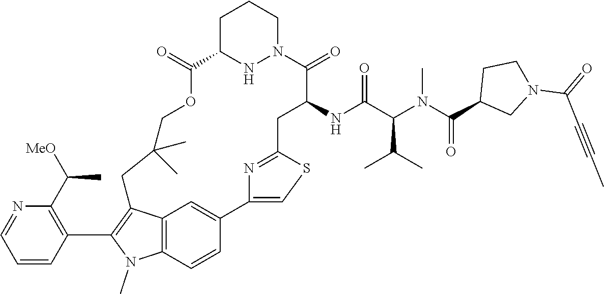 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C)CC(C)(C)COC(=O)[C@@H]3CCCN(N3)C2=O)C(C)C)C1] |
| BB106 | 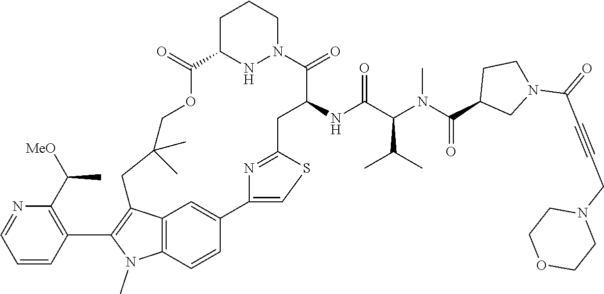 [SMILES: CO[C@@H](C)c1ncccc1-c1c2c3cc(ccc3n1C)-c1csc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB107 | 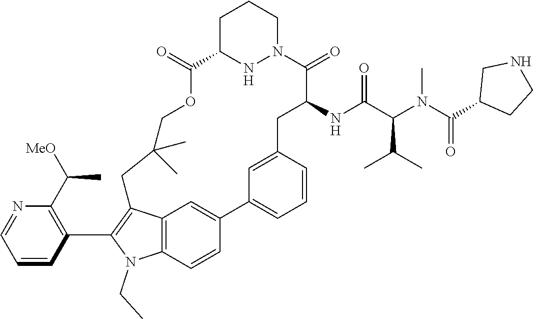 |
| BB109 | 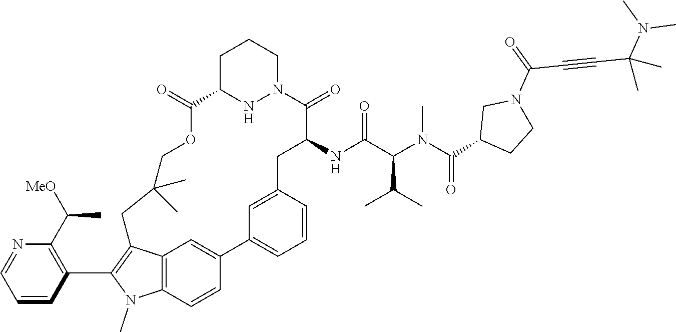 |
| BB111 | 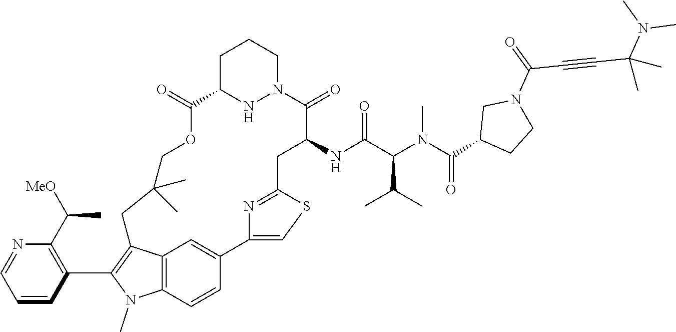 |
| BB112 | 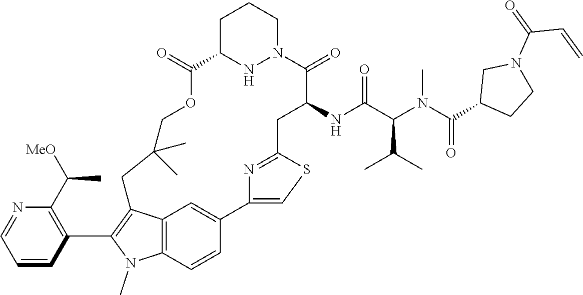 |
| BB113 | 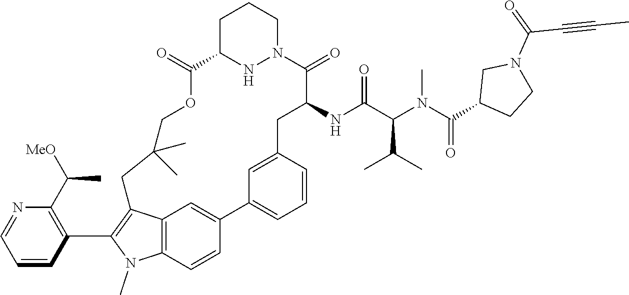 |
| BB115 | 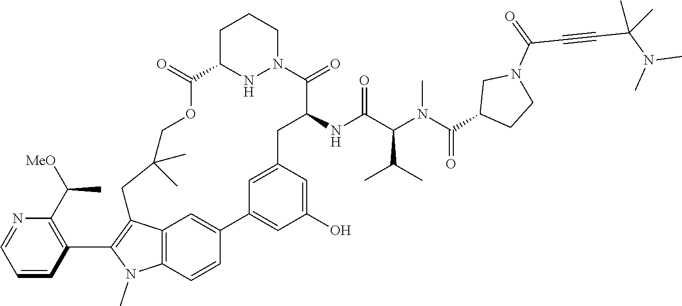 |
| BB116 | 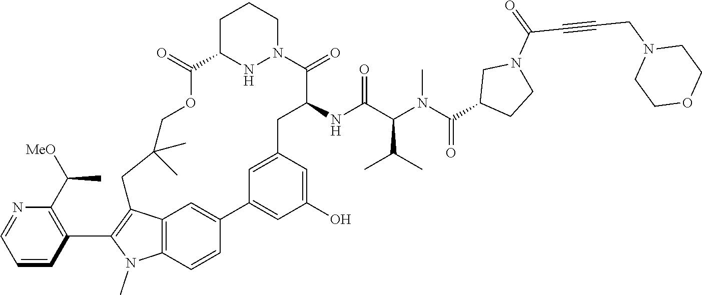 |
| BB117 | 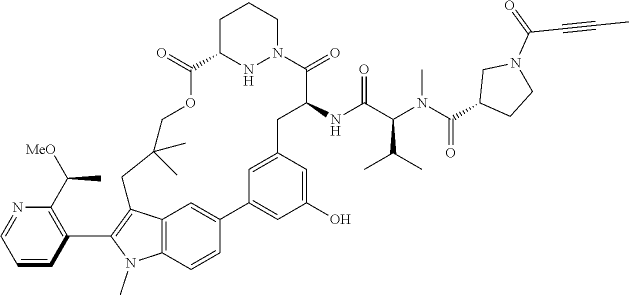 |
| BB118 | 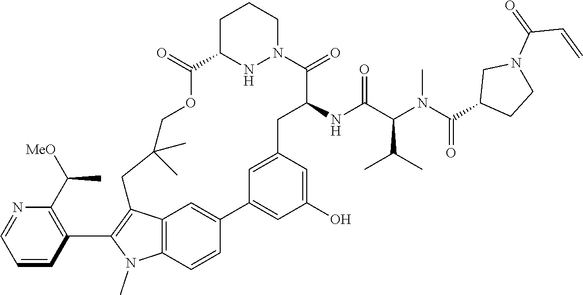 |
| BB119 | 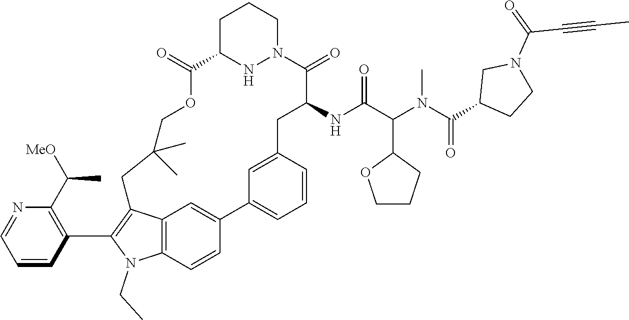 |
| BB120 | 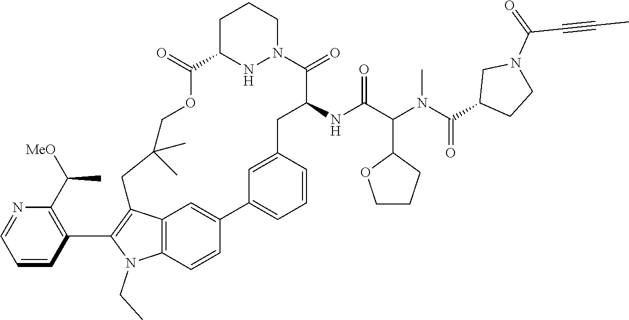 |
| BB121 | 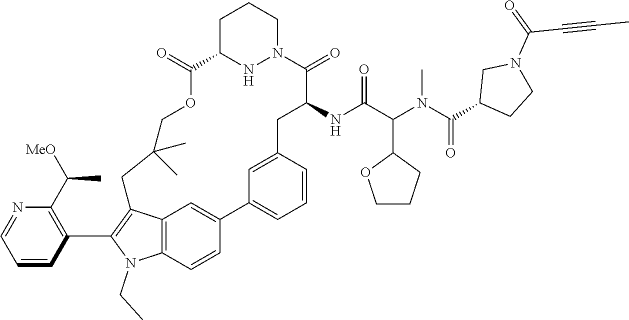 |
| BB122 | 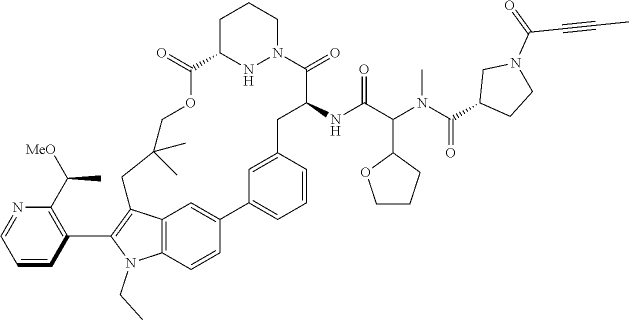 |
| BB123 | 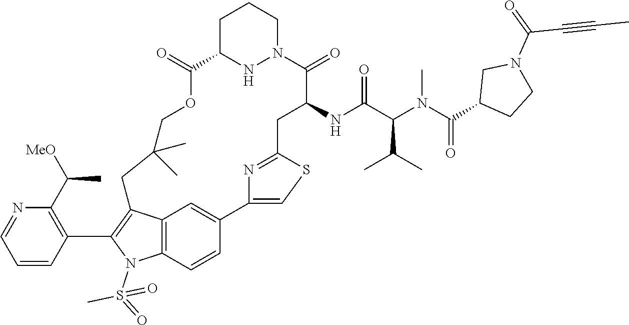 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4S(C)(=O)=O)CC(C)(C)COC(=O)[C@@H]3CCCN(N3)C2=O)C(C)C)C1] |
| BB124 | 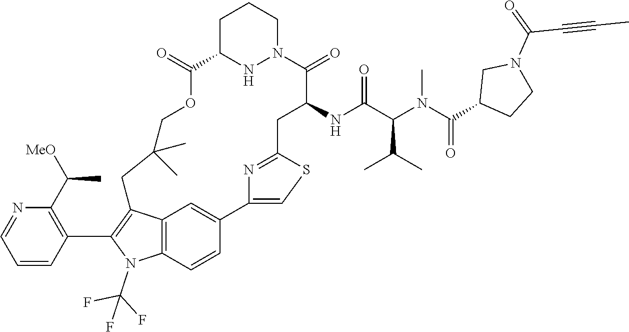 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(F)(F)F)CC(C)(C)COC(=O)[C@@H]3CCCN(N3)C2=O)C(C)C)C1] |
| BB126 | 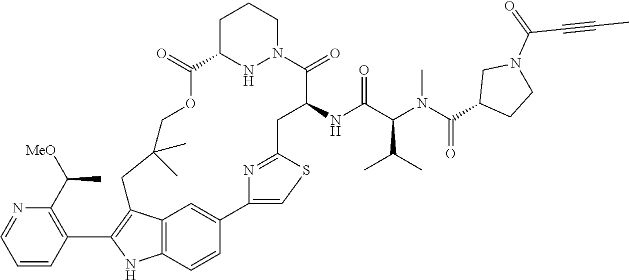 [SMILES: CC#CC(=O)N1CC[C@H](C(=O)N(C)[C@H](C(=O)N[C@H]2Cc3nc(cs3)-c3ccc4[nH]c(-c5cccnc5[C@H](C)OC)c(c4c3)CC(C)(C)COC(=O)[C@@H]3CCCN(N3)C2=O)C(C)C)C1] |
| BB127 | 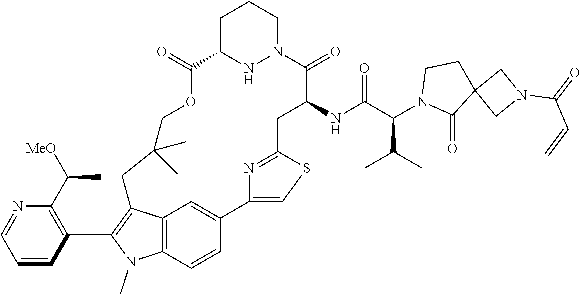 |
| BB128 | 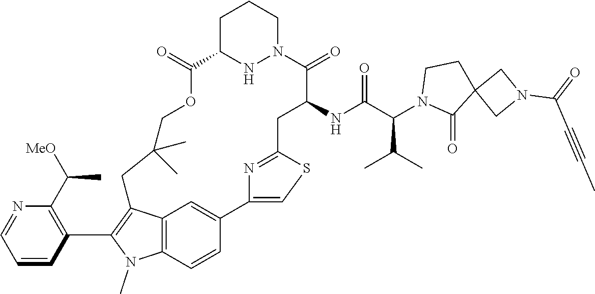 |
| BB129 | 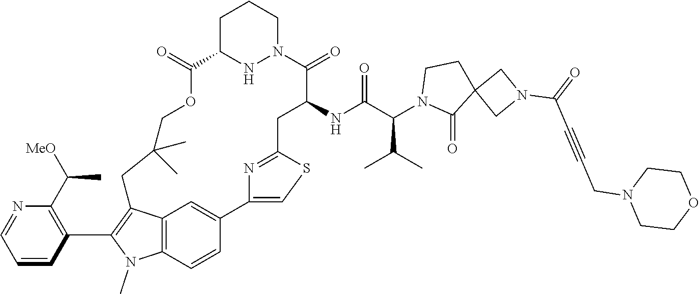 |
| BB130 | 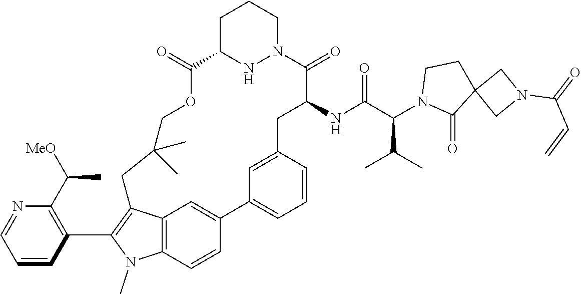 |
| BB131 | 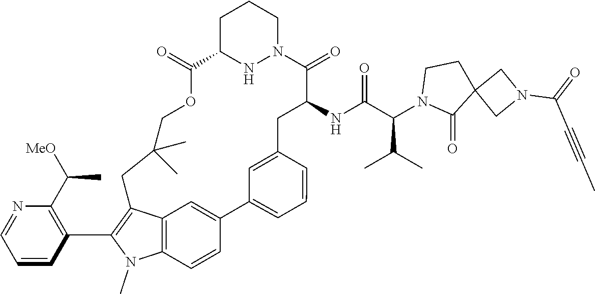 |
| BB132 | 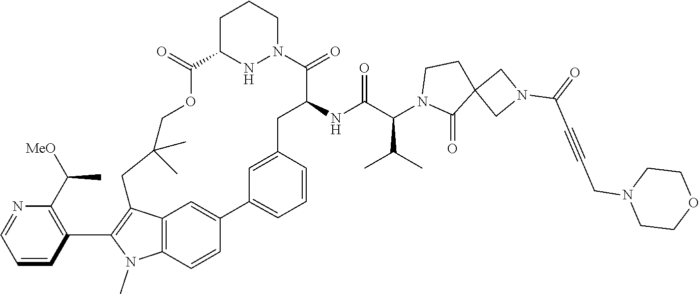 |
| BB139 | 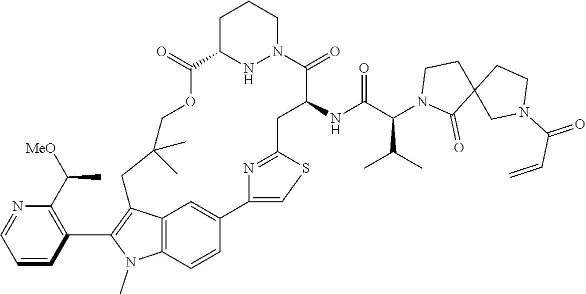 |
| BB140 | 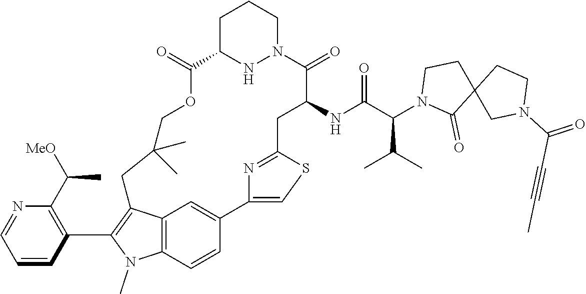 |
| BB141 | 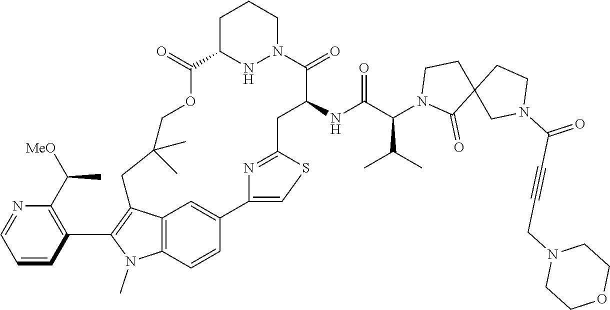 |
| BB142 | 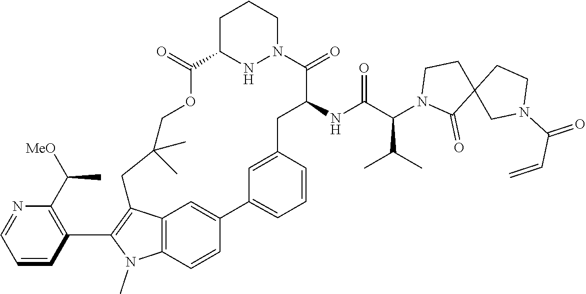 |
| BB143 | 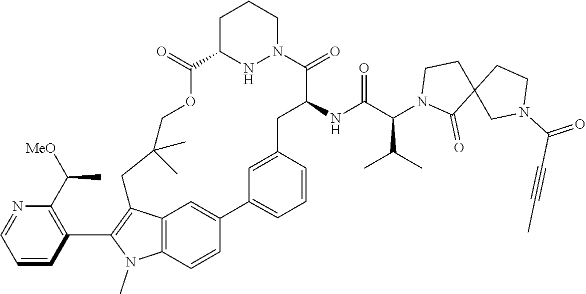 |
| BB144 | 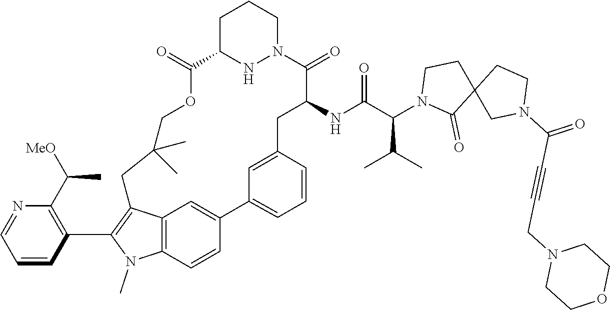 |
| BB145 | 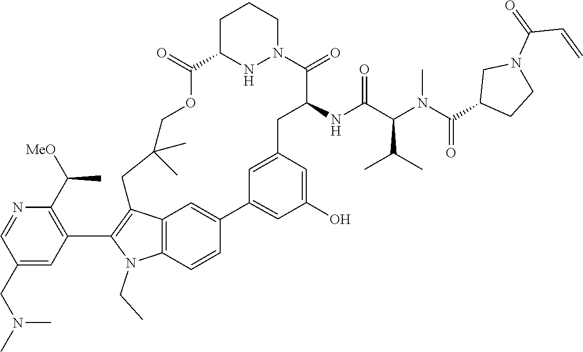 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB146 | 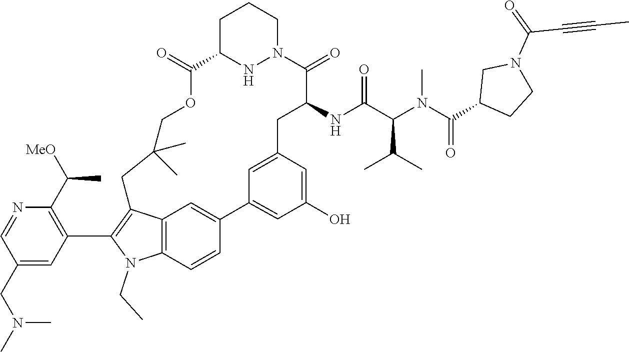 [SMILES: CC#CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB147 | 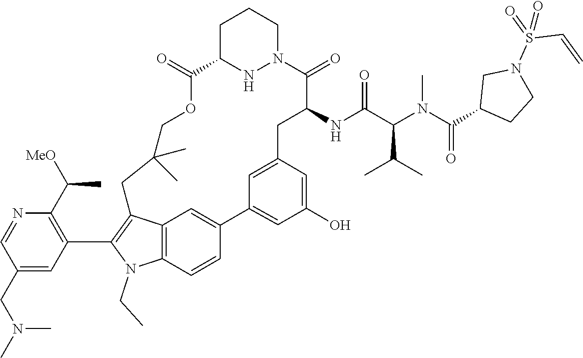 [SMILES: C=CS(=O)(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB148 | 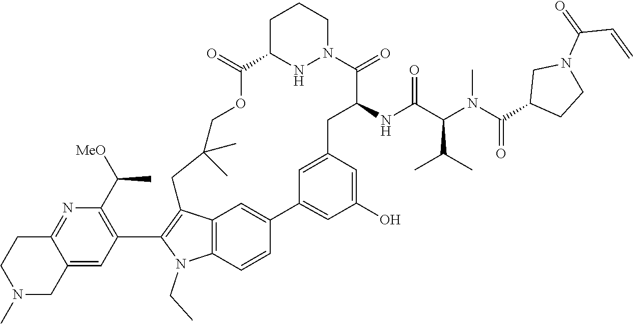 [SMILES: C=CC(=O)N1CC[C@H](C(=O)N(C)[C@H](C(=O)N[C@H]2Cc3cc(O)cc(c3)-c3ccc4c(c3)c(c(-c3cc5c(nc3[C@H](C)OC)CCN(C)C5)n4CC)CC(C)(C)COC(=O)[C@@H]3CCCN(N3)C2=O)C(C)C)C1] |
| BB149 | 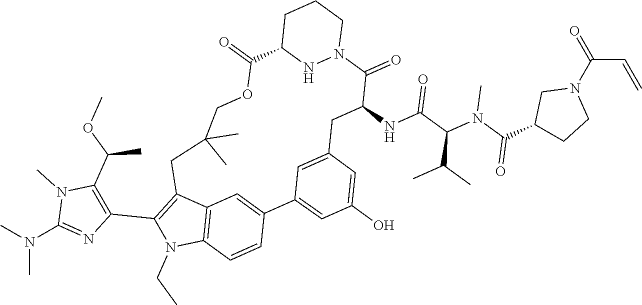 [SMILES: C=CC(=O)N1CC[C@H](C(=O)N(C)[C@H](C(=O)N[C@H]2Cc3cc(O)cc(c3)-c3ccc4c(c3)c(c(-c3nc(N(C)C)n(C)c3[C@H](C)OC)n4CC)CC(C)(C)COC(=O)[C@@H]3CCCN(N3)C2=O)C(C)C)C1] |
| BB150 | 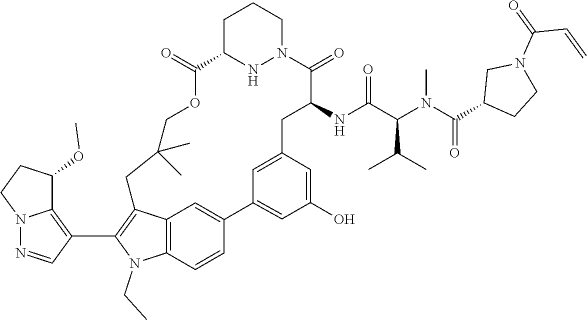 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CC5)n4CC)CC(C)(C)COC(=O)[C@@H]3CCCN(N3)C2=O)C(C)C)C1] |
| BB161 | 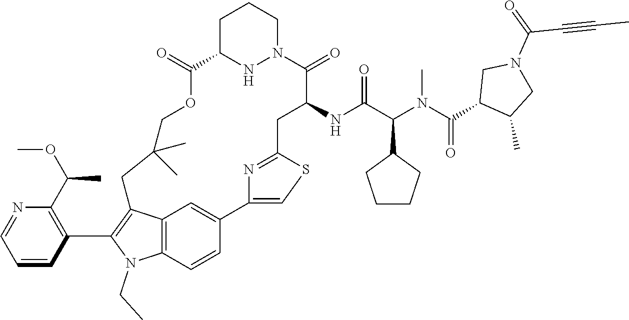 |
| BB162 | 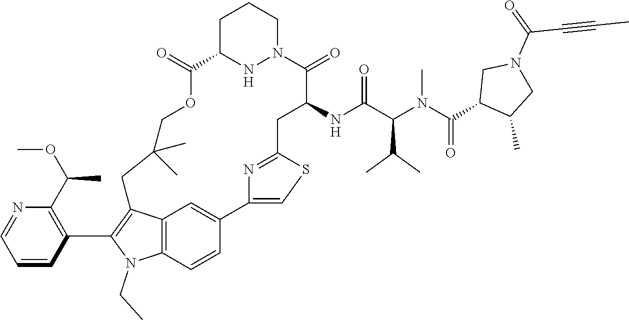 |
| BB163 | 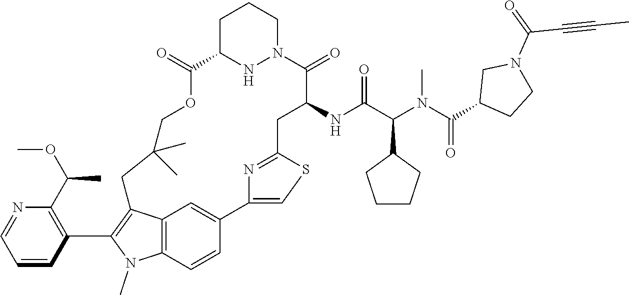 |
| BB164 | 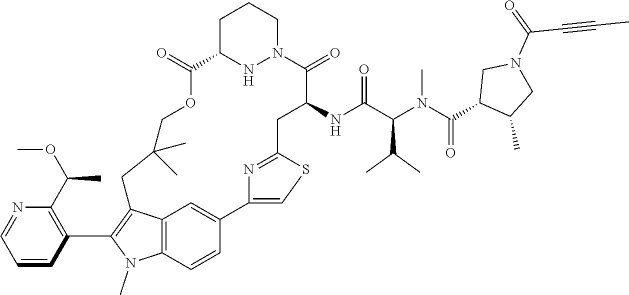 |
| BB165 | 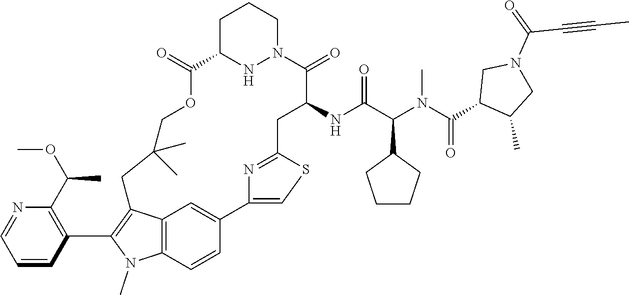 |
| BB167 | 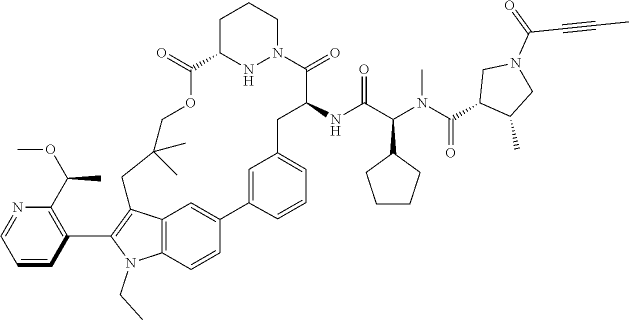 |
| BB168 | 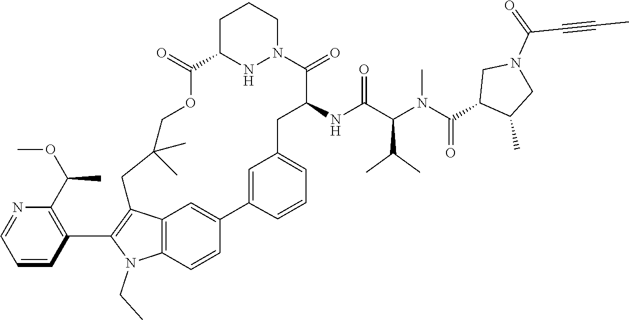 |
| BB169 | 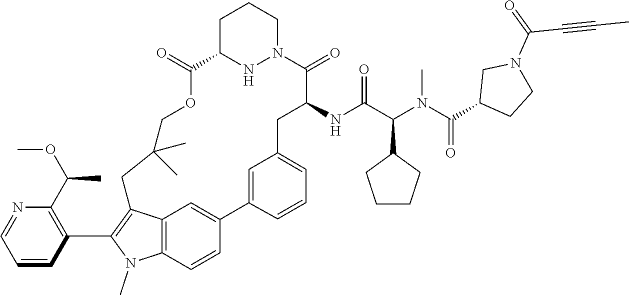 |
| BB170 | 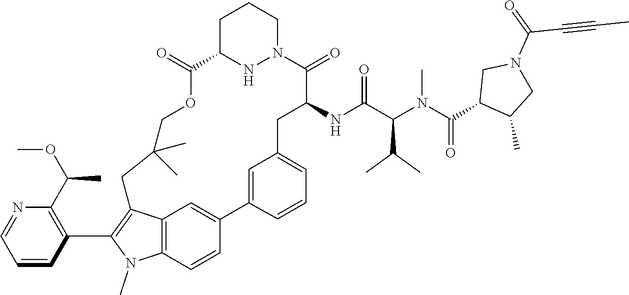 |
| BB171 | 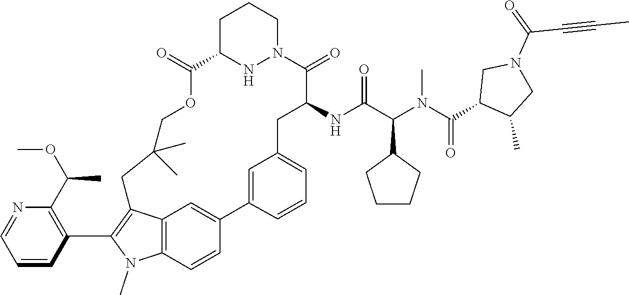 |
| BB172 | 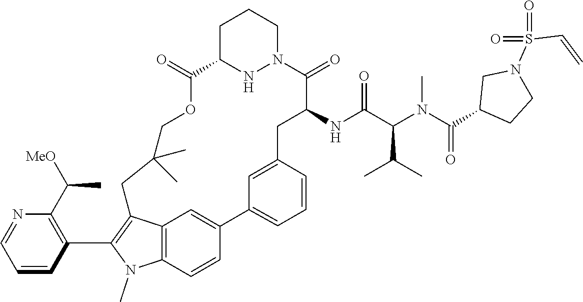 |
| BB173 | 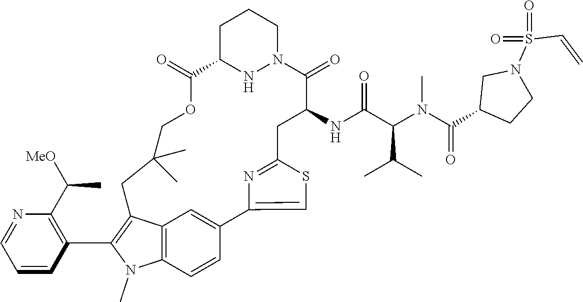 |
| BB174 | 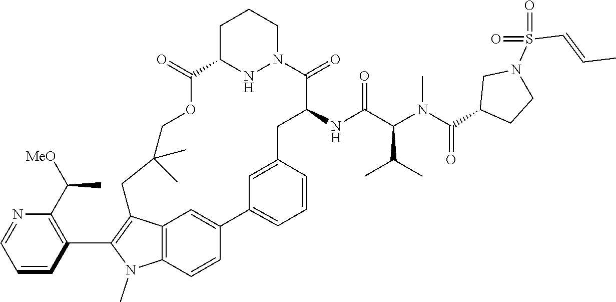 |
| BB175 | 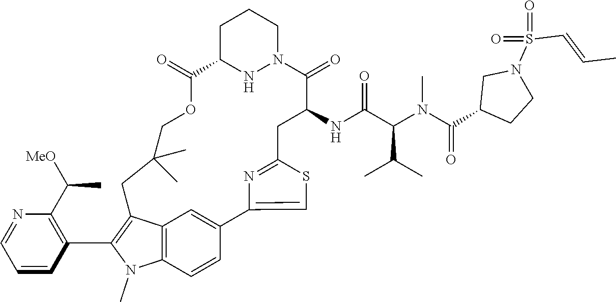 |
| BB176 | 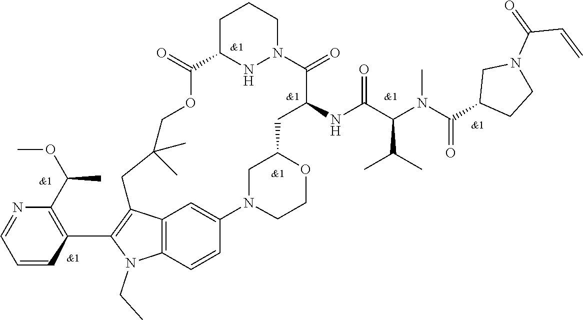 |
| BB177 | 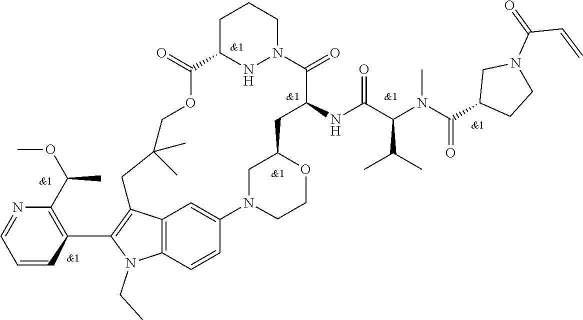 |
| BB178 | 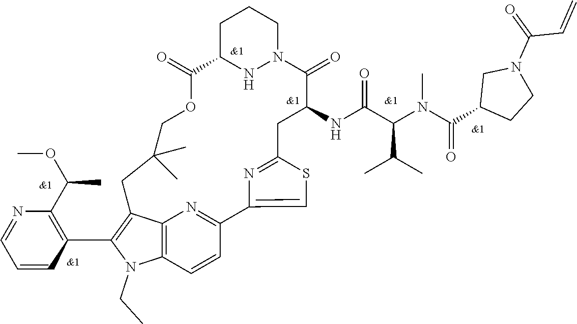 |
| BB179 | 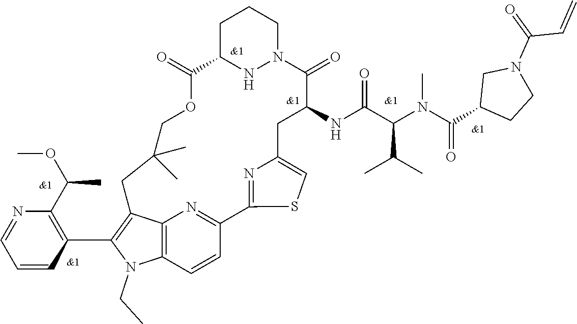 |
| BB180 | 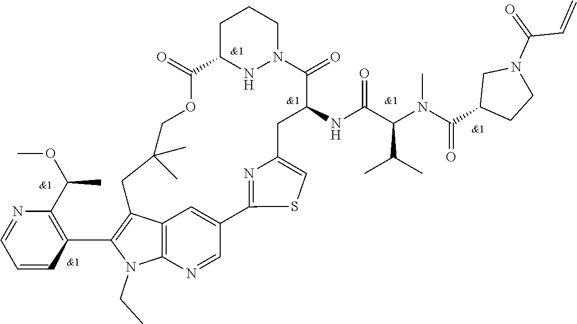 |
| BB181 | 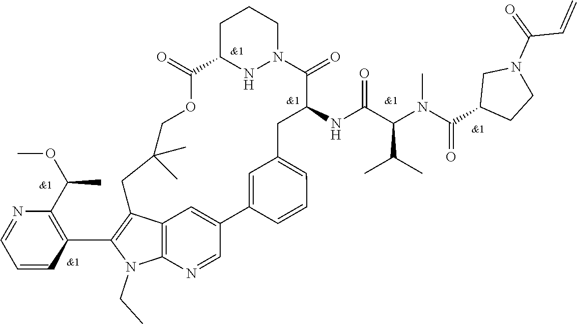 |
| BB182 | 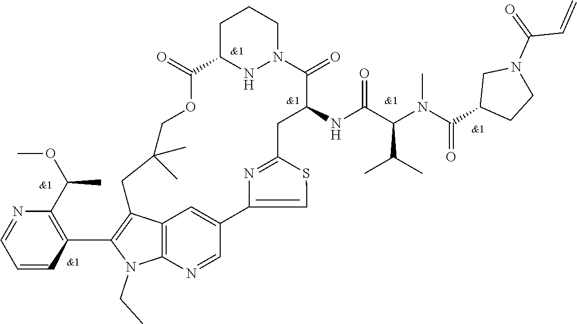 |
| BB183 | 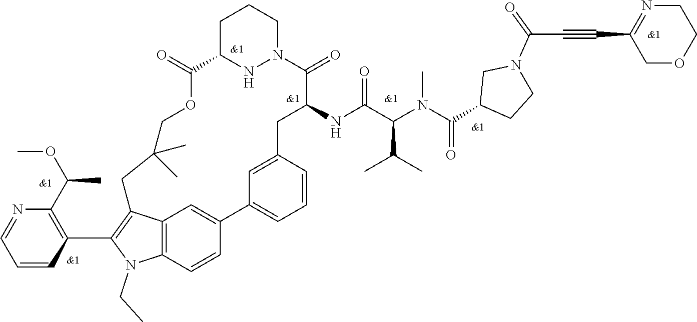 |
| BB184 | 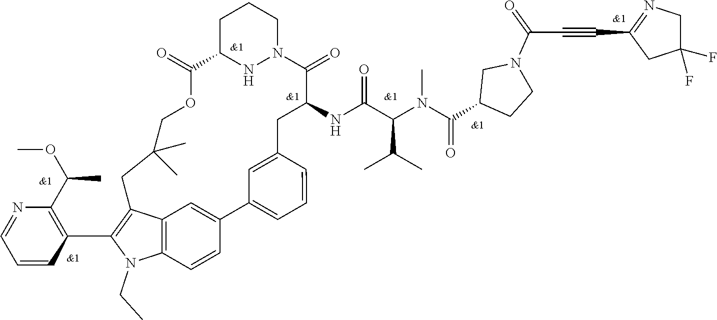 |
| BB185 | 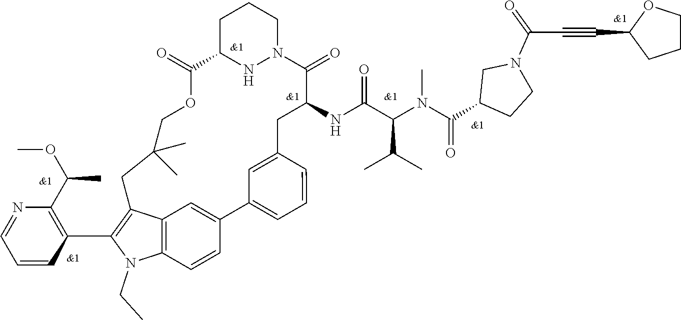 |
| BB186 | 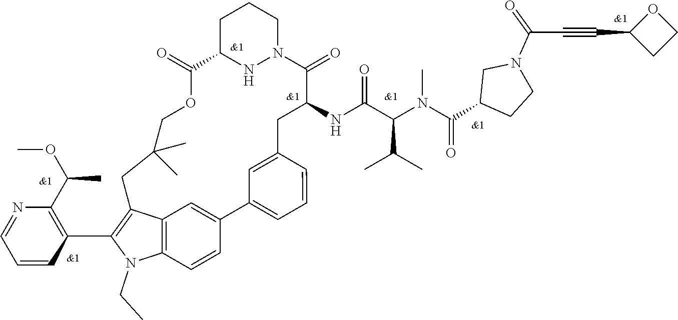 |
| BB187 | 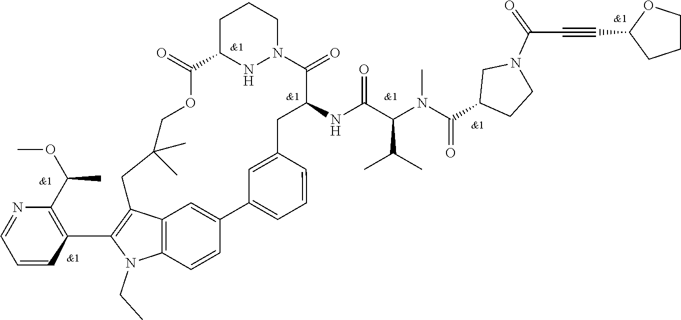 |
| BB188 | 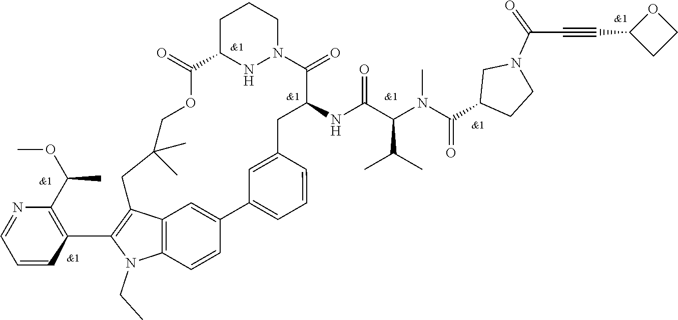 |
| BB189 | 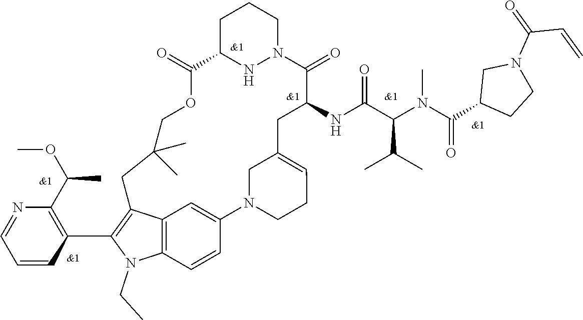 |
| BB190 | 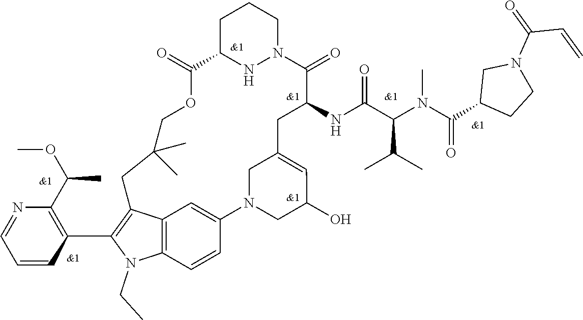 |
| BB191 | 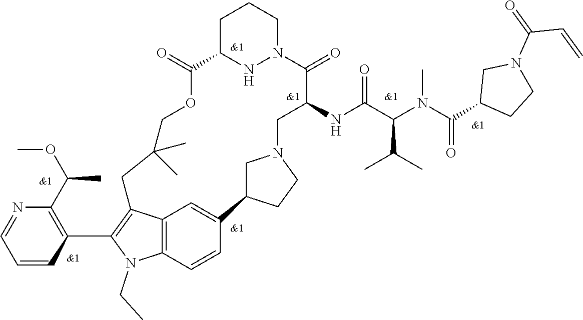 |
| BB192 | 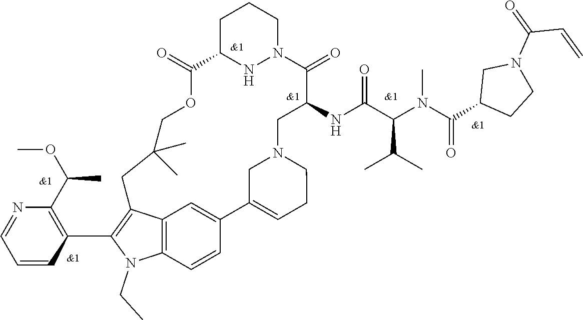 |
| BB194 | 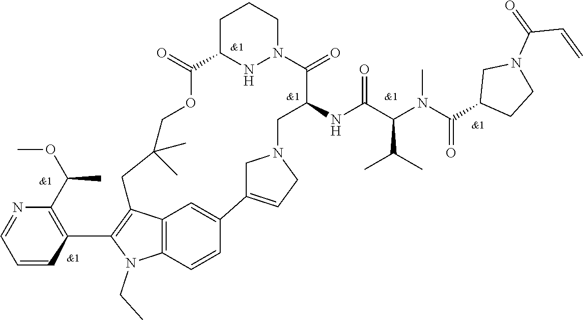 |
| BB195 | 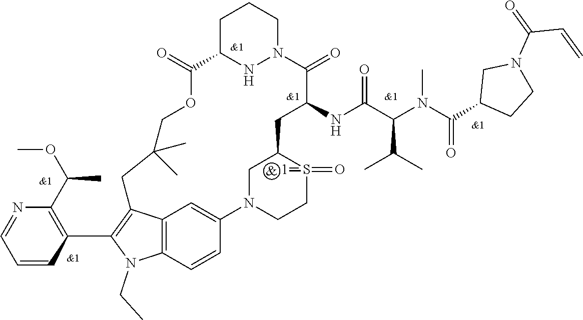 |
| BB196 | 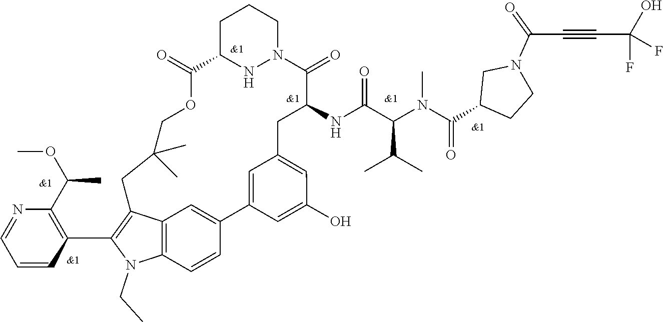 |
| BB197 | 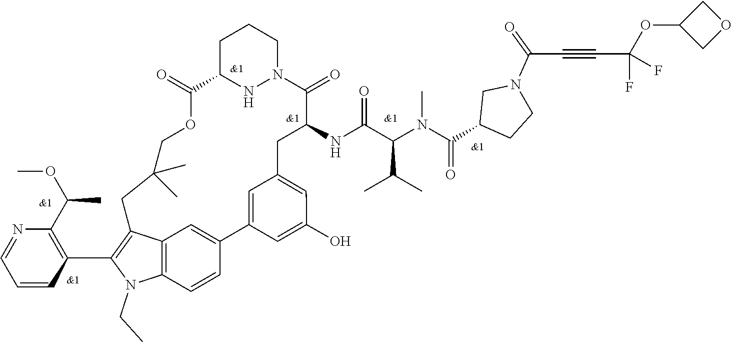 |
| BB198 | 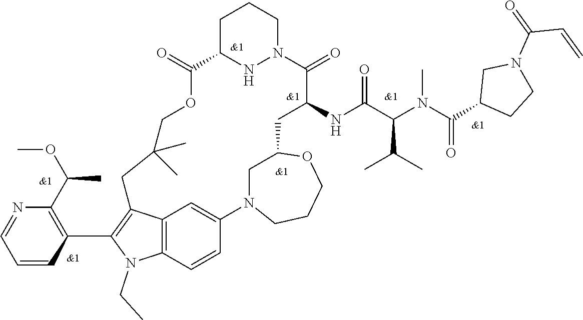 |
| BB199 | 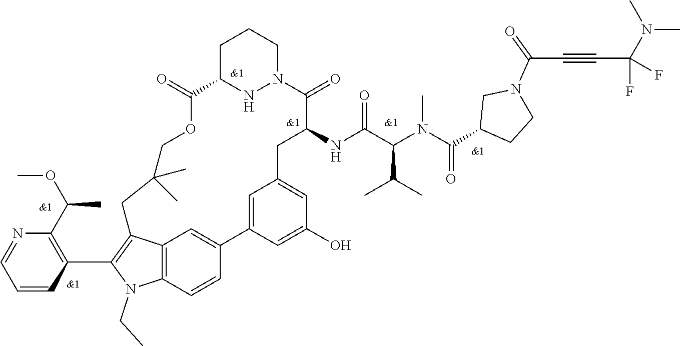 |
| BB200 | 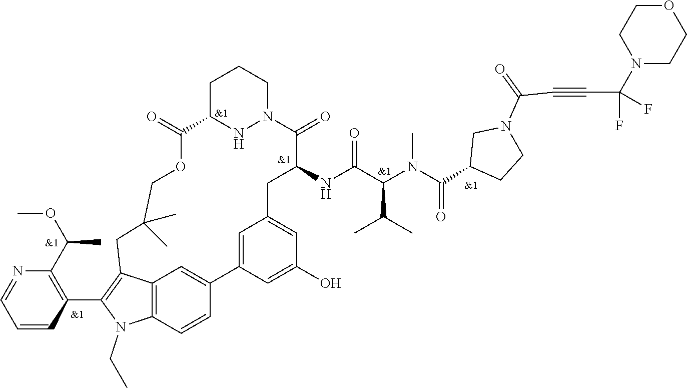 |
| BB201 | 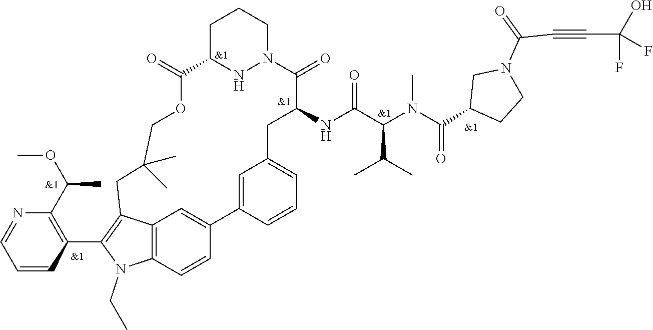 |
| BB202 | 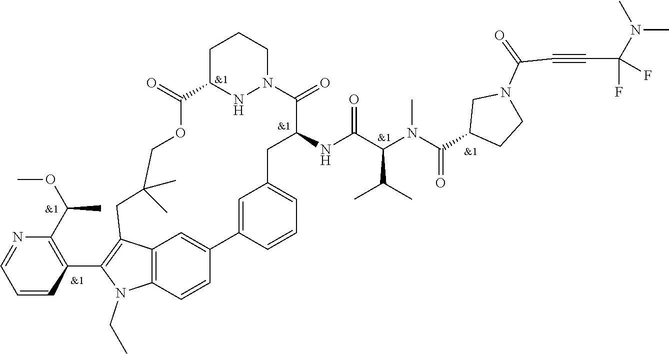 |
| BB203 | 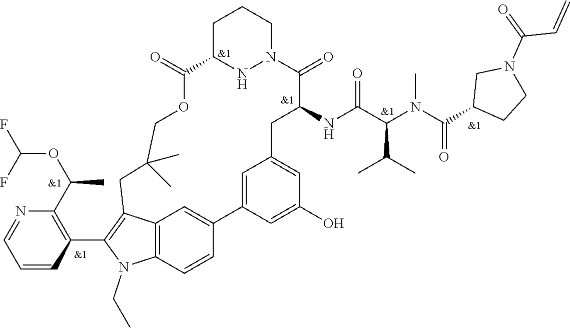 |
| BB204 | 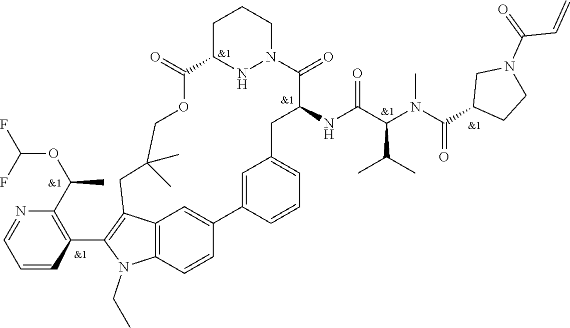 |
| BB205 | 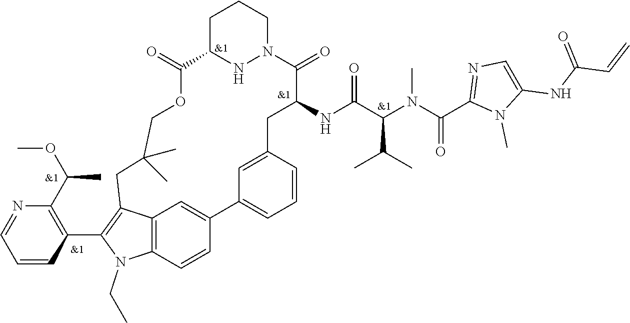 |
| BB206 | 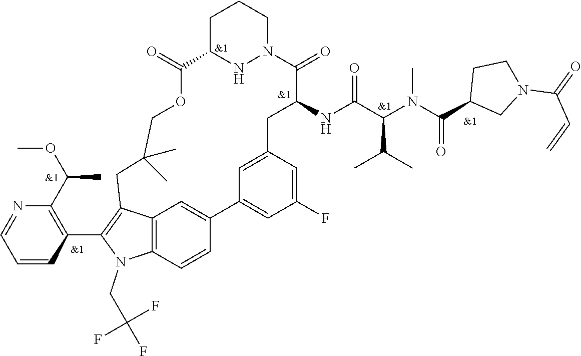 |
| BB207 | 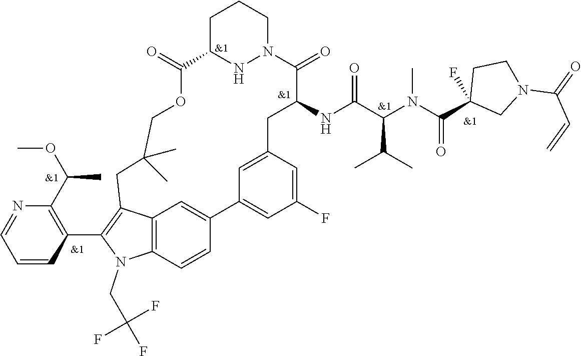 |
| BB208 | 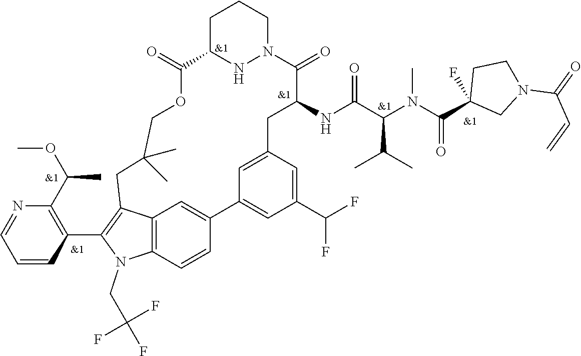 |
| BB209 | 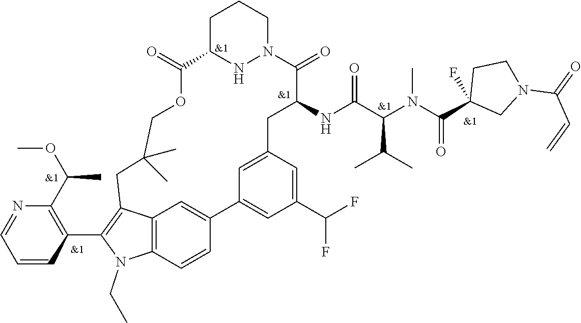 |
| BB210 | 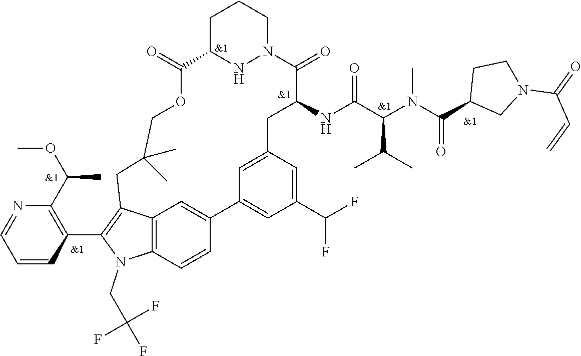 |
| BB211 | 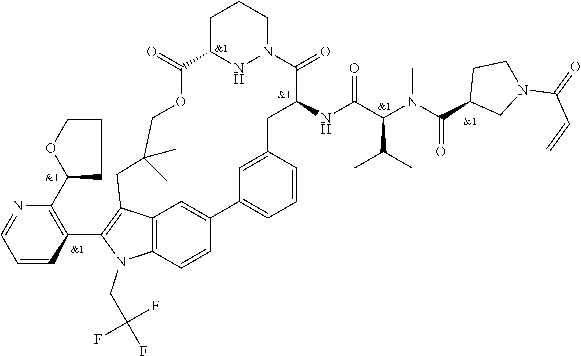 |
| BB212 | 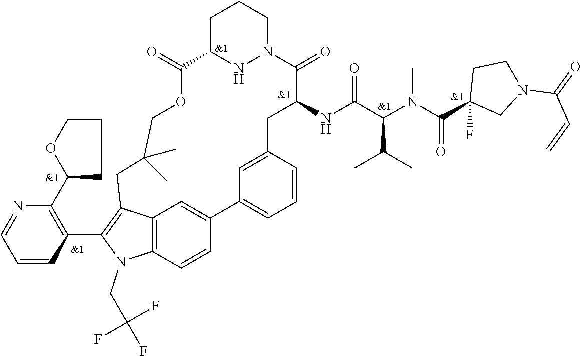 |
| BB213 | 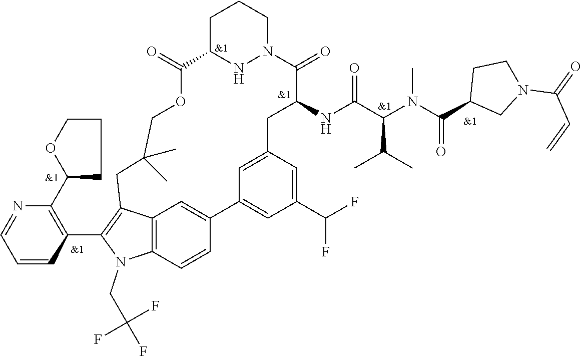 |
| BB214 | 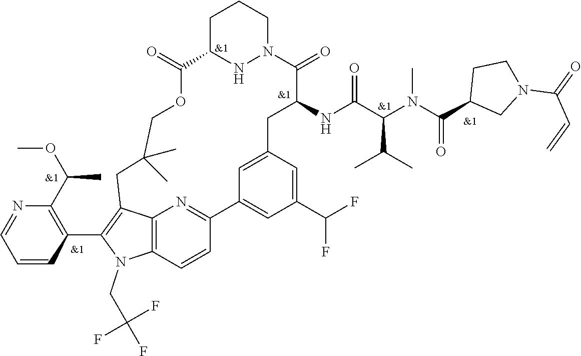 |
| BB215 | 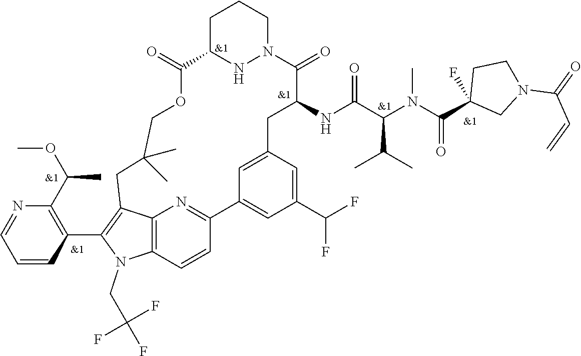 |
| BB216 | 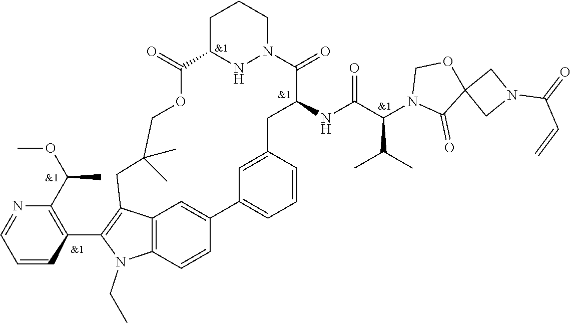 |
| BB217 | 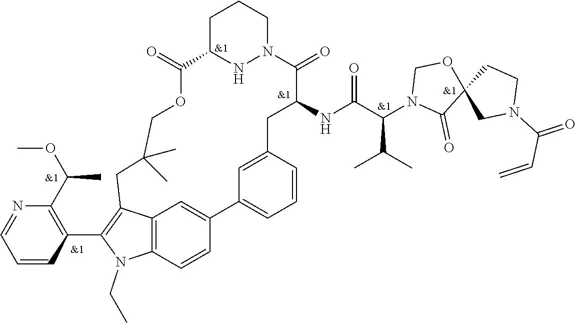 |
| BB218 | 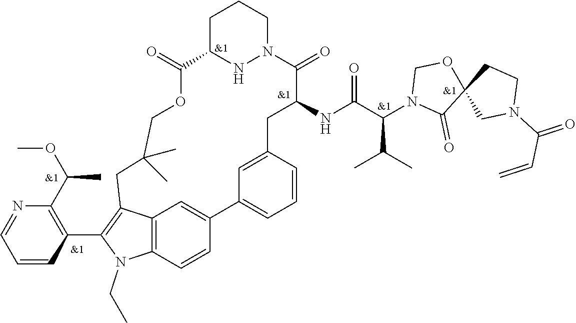 |
| BB219 | 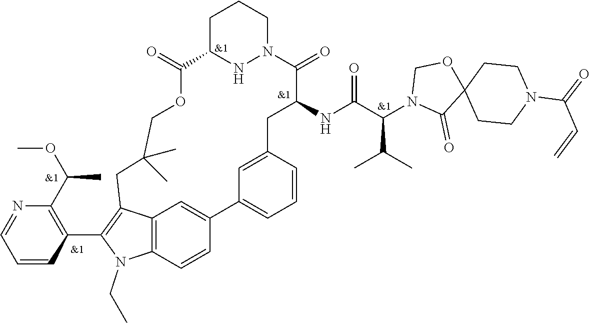 |
| BB220 | 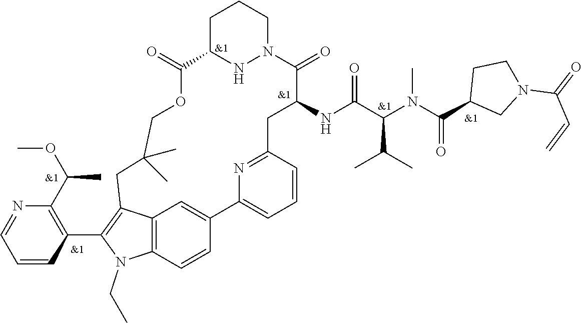 |
| BB221 | 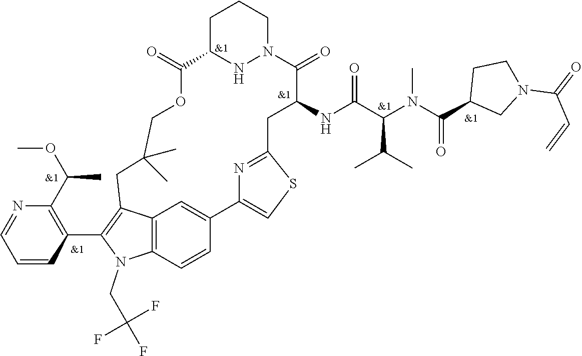 |
| BB222 | 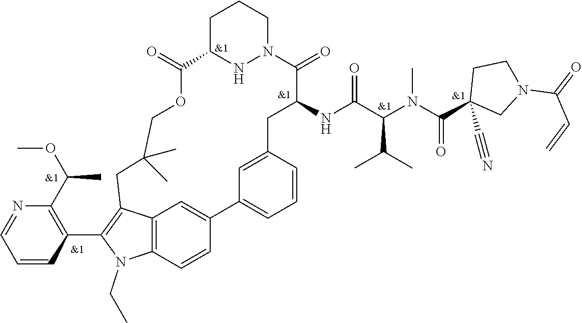 |
| BB223 | 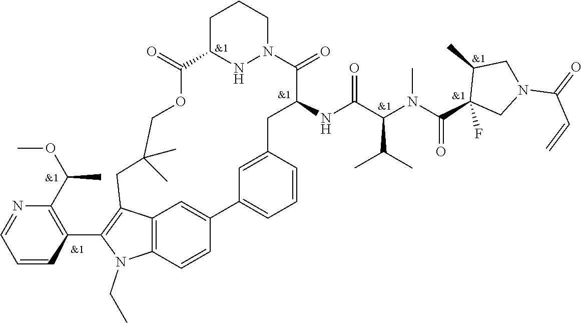 |
| BB224 | 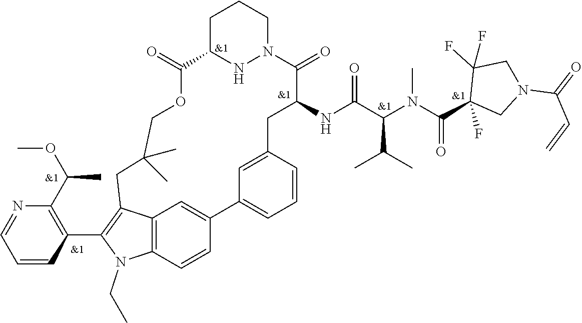 |
| BB225 | 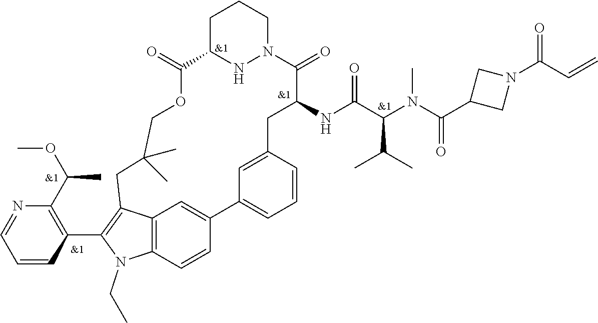 |
| BB226 | 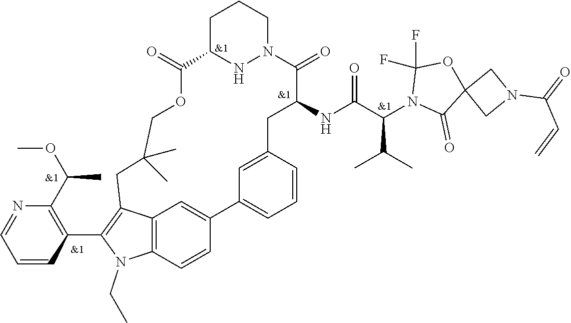 |
| BB227 | 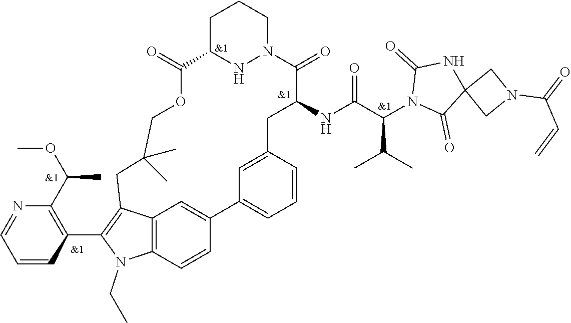 |
| BB228 | 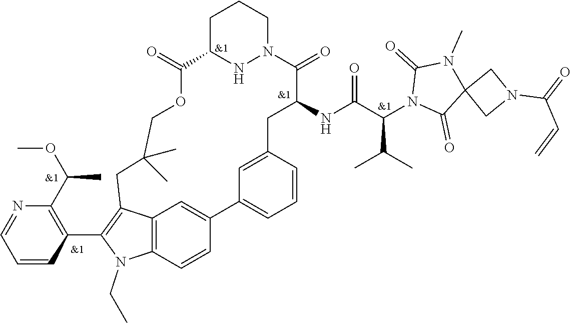 |
| BB229 | 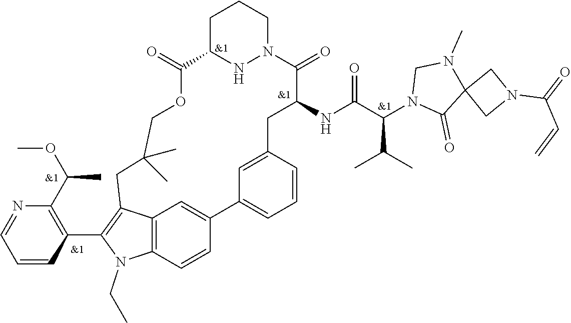 |
| BB230 | 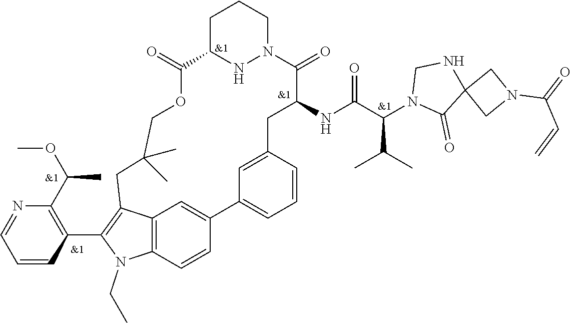 |
| BB231 | 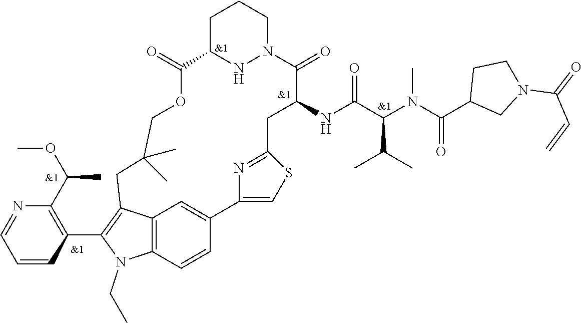 |
| BB232 | 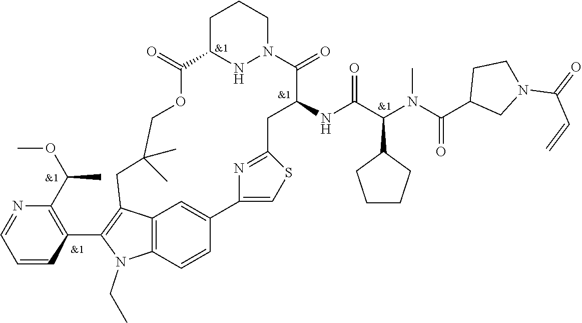 |
| BB233 | 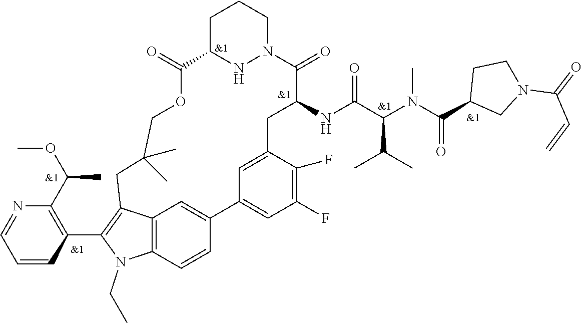 |
| BB234 | 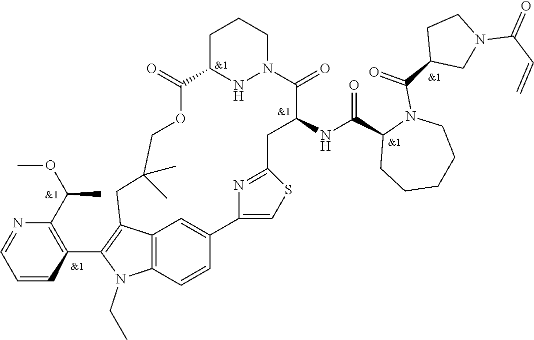 |
| BB235 | 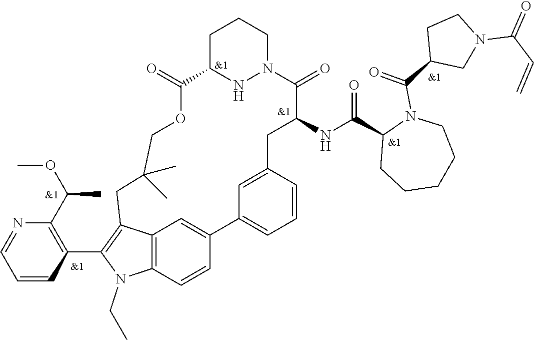 |
| BB236 | 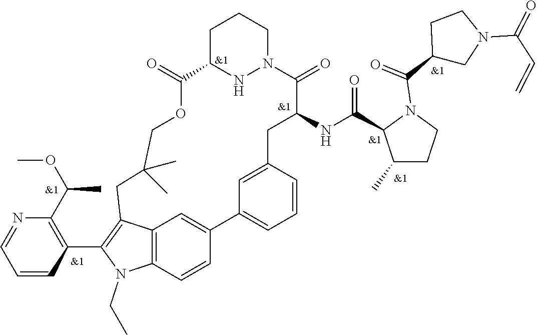 |
| BB237 | 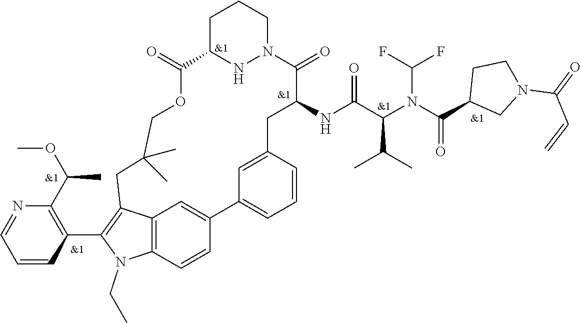 |
| BB238 | 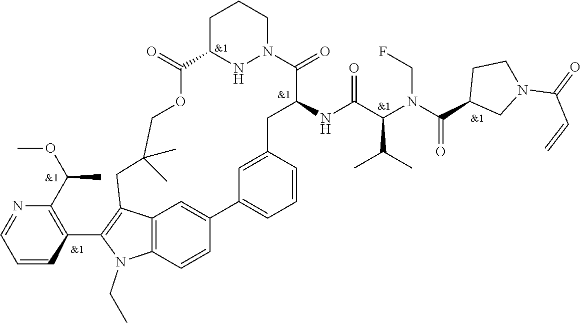 |
| BB239 | 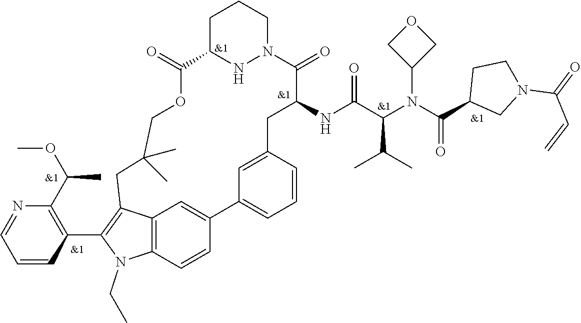 |
| BB240 | 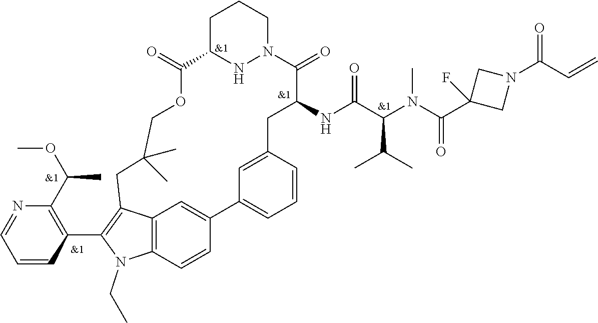 |
| BB241 | 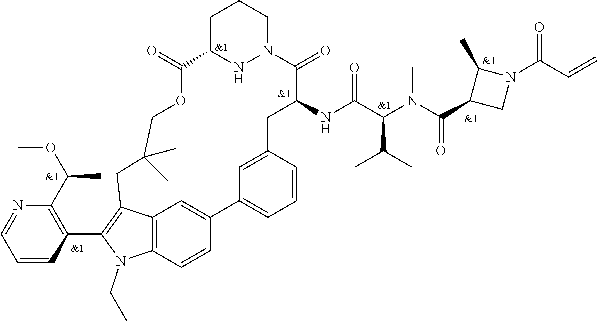 |
| BB242 | 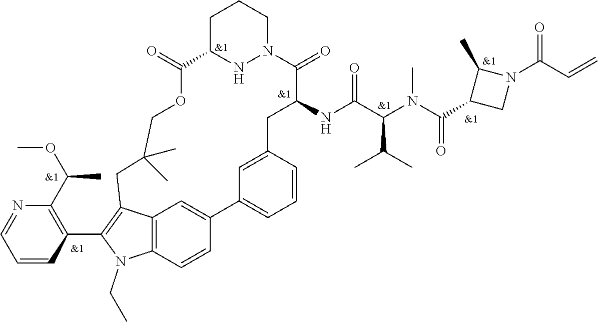 |
| BB243 | 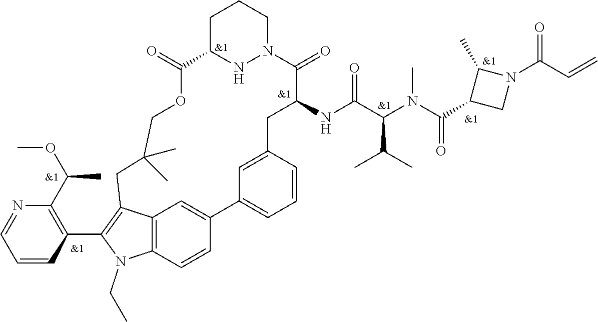 |
| BB244 | 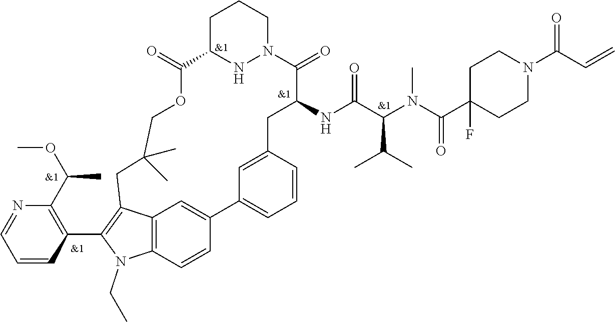 |
| BB245 | 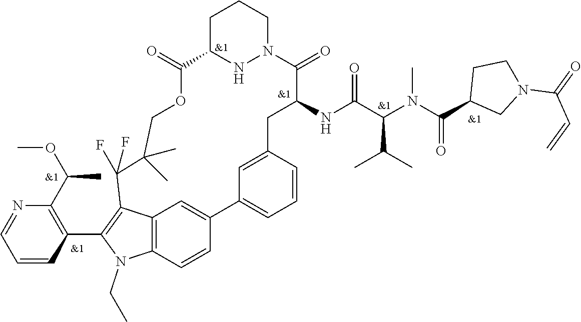 |
| BB246 | 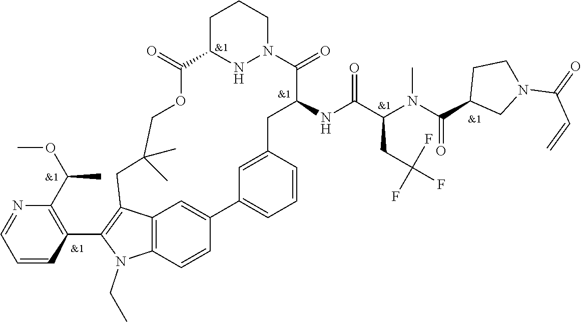 |
| BB247 | 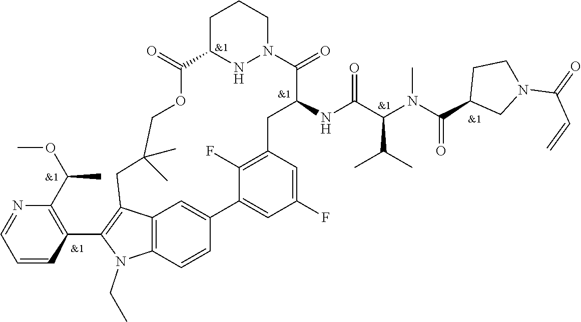 |
| BB248 | 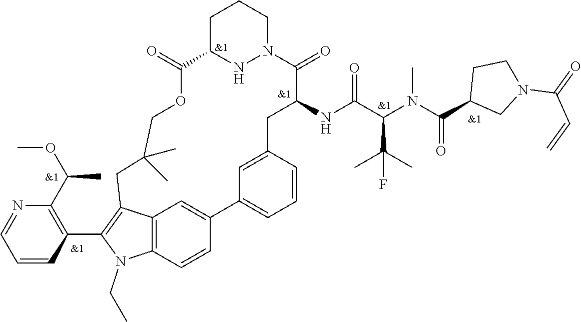 |
| BB249 | 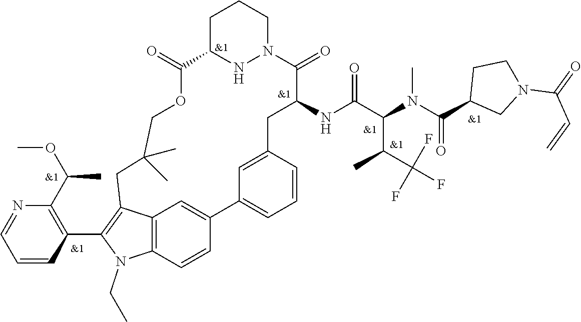 |
| BB250 | 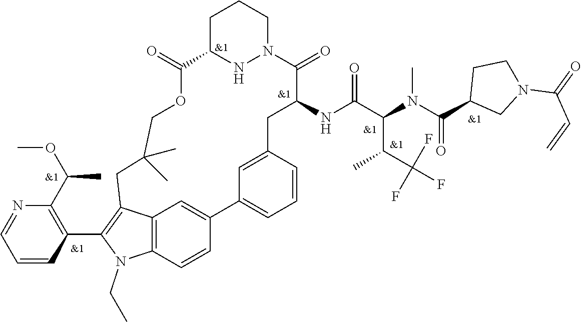 |
| BB251 | 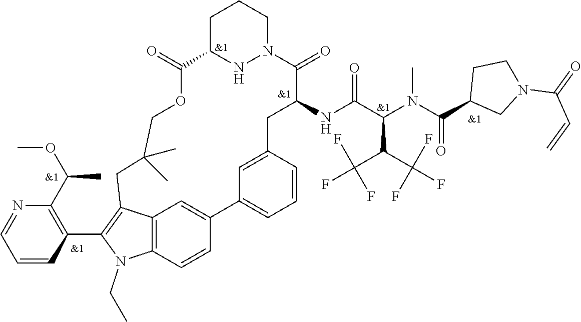 |
| BB252 | 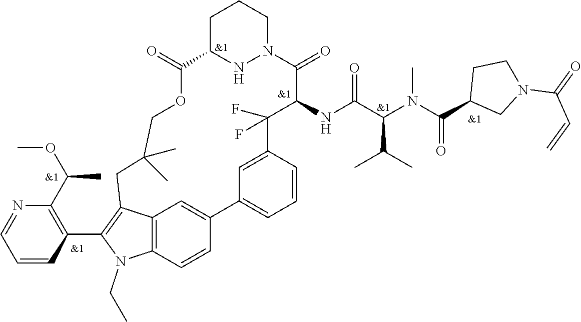 |
| BB253 | 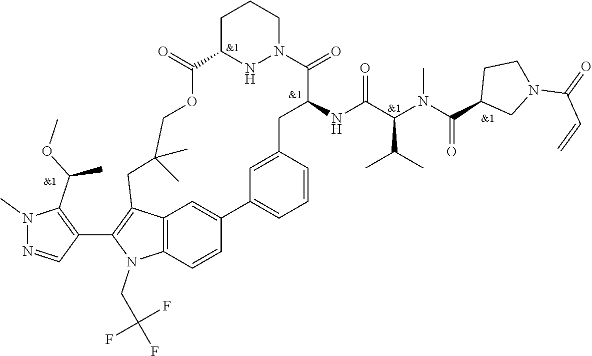 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| BB254 | 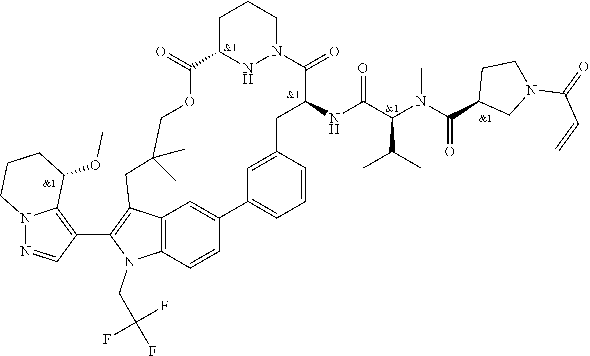 [SMILES: C=CC(=O)N1CC[C@H](C(=O)N(C)[C@H](C(=O)N[C@H]2Cc3cccc(c3)-c3ccc4c(c3)c(c(-c3cnn5c3[C@@H](OC)CCC5)n4CC(F)(F)F)CC(C)(C)COC(=O)[C@@H]3CCCN(N3)C2=O)C(C)C)C1] |
| BB255 | 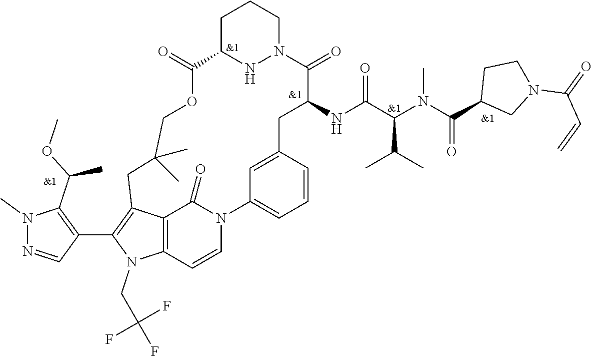 [SMILES: C=CC(=O)N1CC[C@H](C(=O)N(C)[C@H](C(=O)N[C@H]2Cc3cccc(c3)-n3ccc4c(c(c(-c5cnn(C)c5[C@H](C)OC)n4CC(F)(F)F)CC(C)(C)COC(=O)[C@@H]4CCCN(N4)C2=O)c3=O)C(C)C)C1] |
| BB256 | 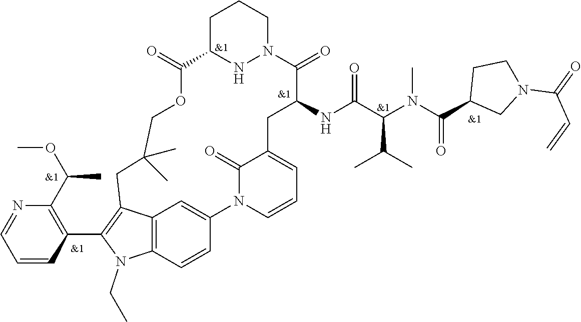 |
| BB257 | 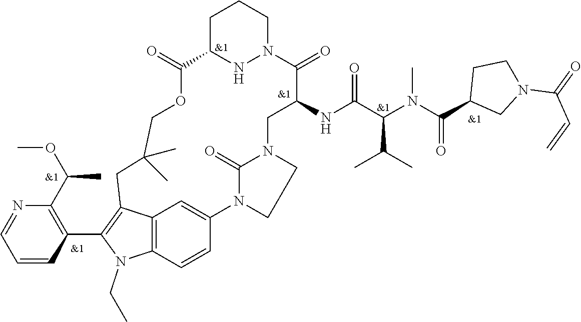 |
| BB258 | 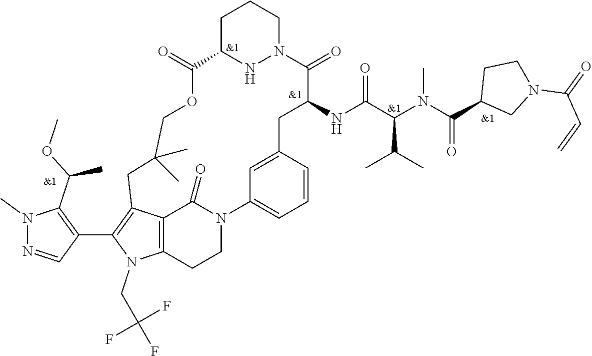 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3=O)C(C)C)C1] |
| BB259 | 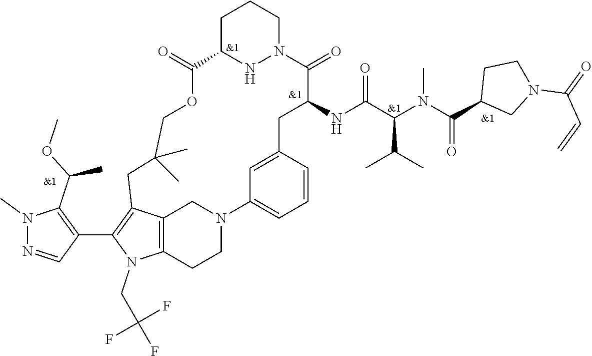 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB260 | 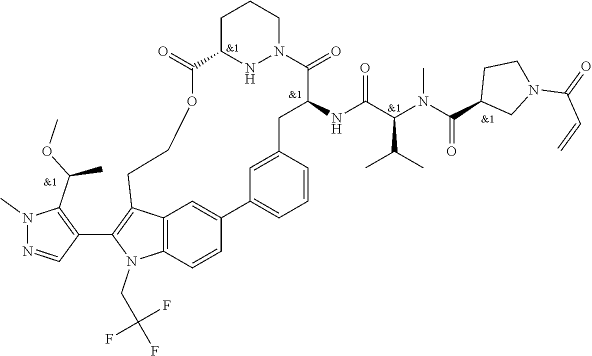 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CCOC(=O)[C@@H]3CCCN(N3)C2=O)C(C)C)C1] |
| BB261 | 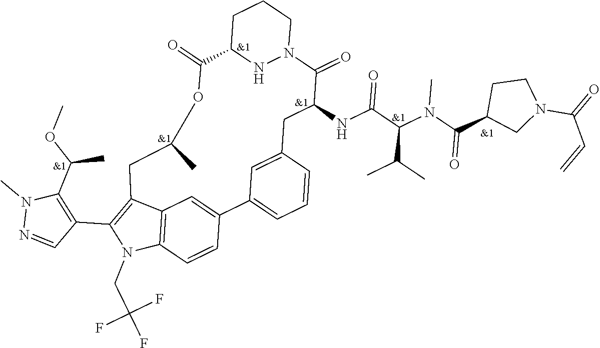 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@H](C)OC(=O)[C@@H]3CCCN(N3)C2=O)C(C)C)C1] |
| BB262 | 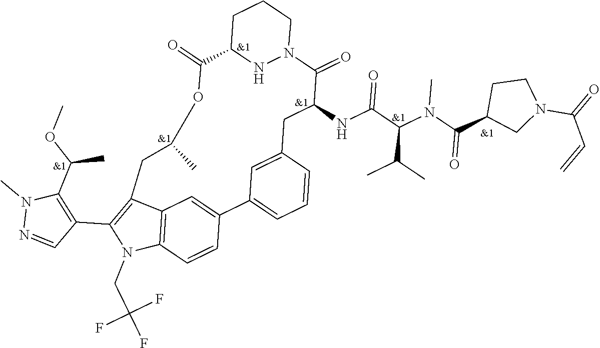 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@@H](C)OC(=O)[C@@H]3CCCN(N3)C2=O)C(C)C)C1] |
| BB263 | 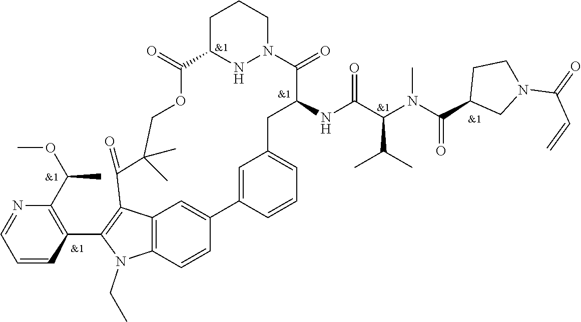 |
| BB264 | 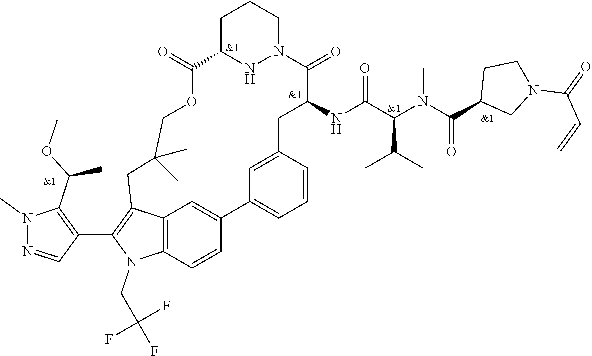 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| BB265 | 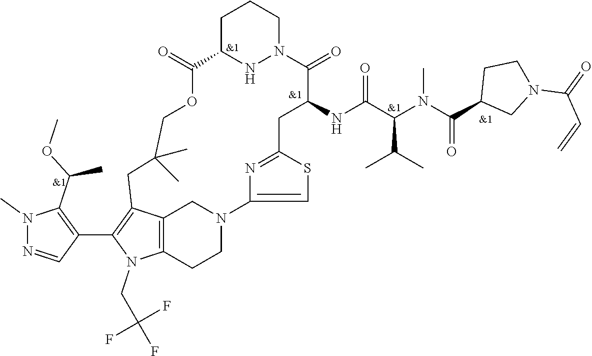 [SMILES: C=CC(=O)N1CC[C@H](C(=O)N(C)[C@H](C(=O)N[C@H]2Cc3nc(cs3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB266 | 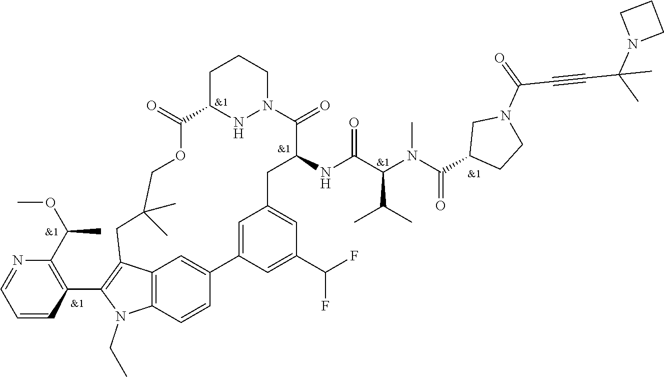 |
| BB267 | 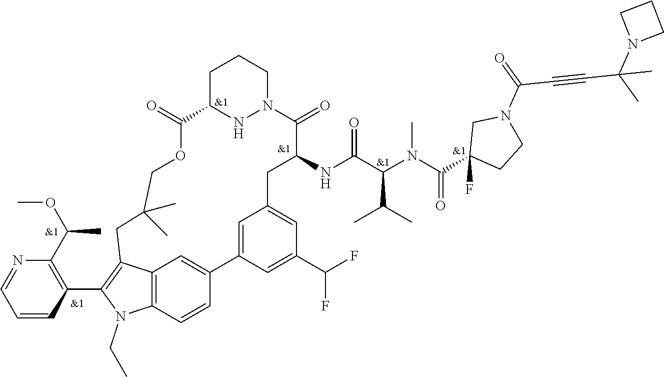 |
| BB268 | 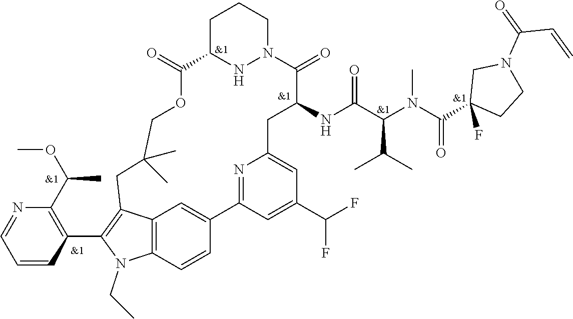 |
| BB269 | 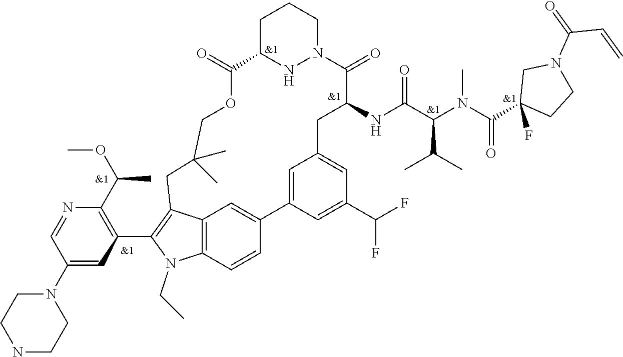 |
| BB270 | 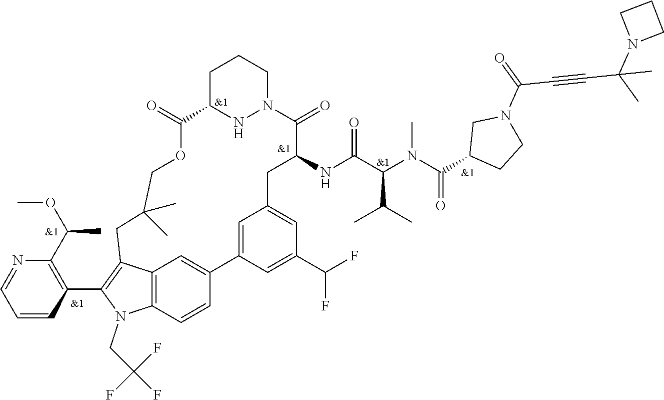 |
| BB271 | 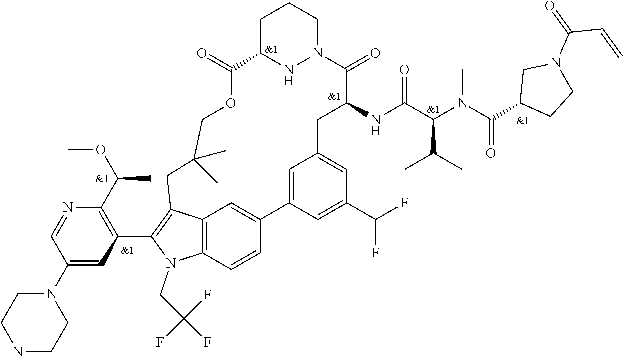 |
| BB272 | 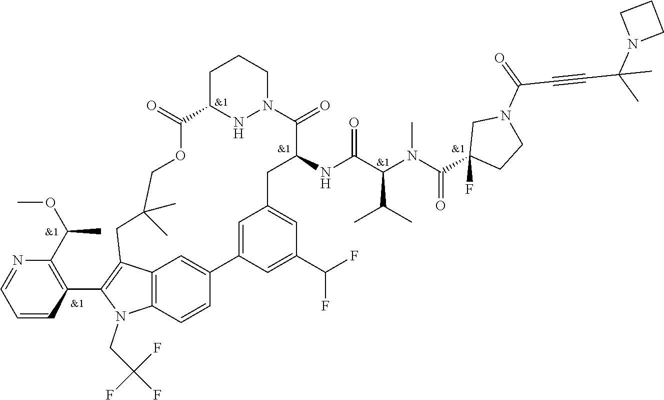 |
| BB273 | 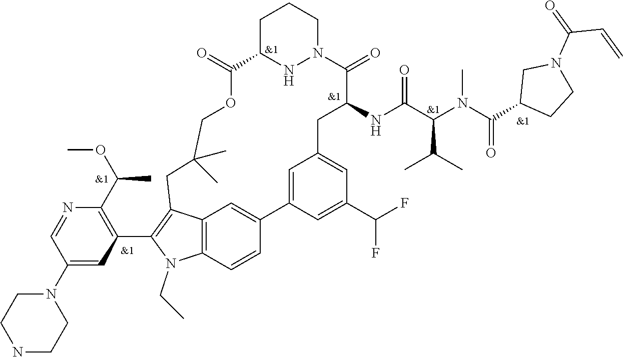 |
| BB274 | 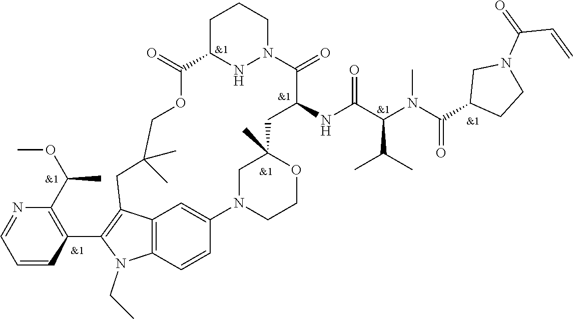 |
| BB275 | 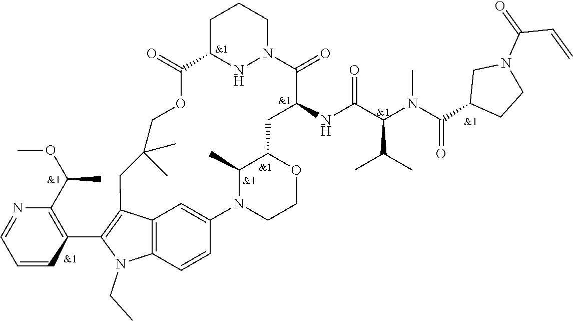 |
| BB276 | 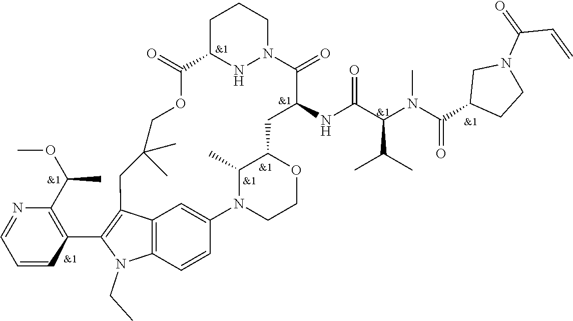 |
| BB277 | 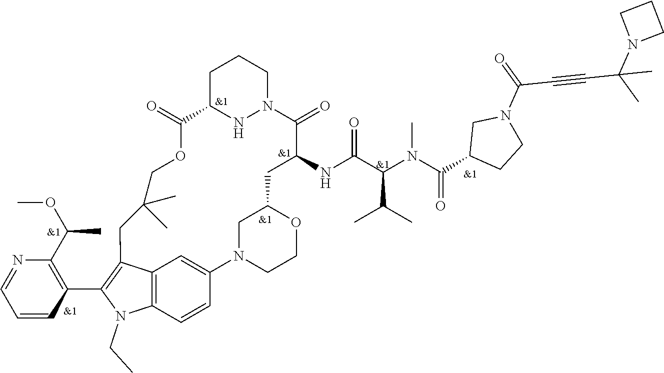 |
| BB278 | 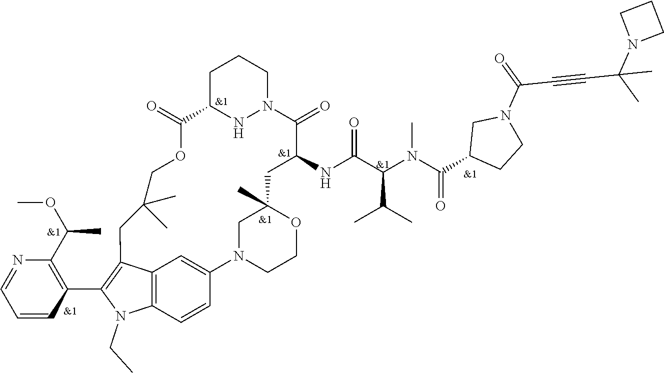 |
| BB279 | 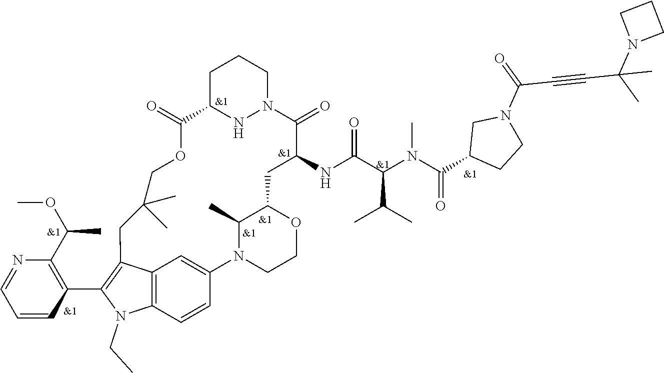 |
| BB280 | 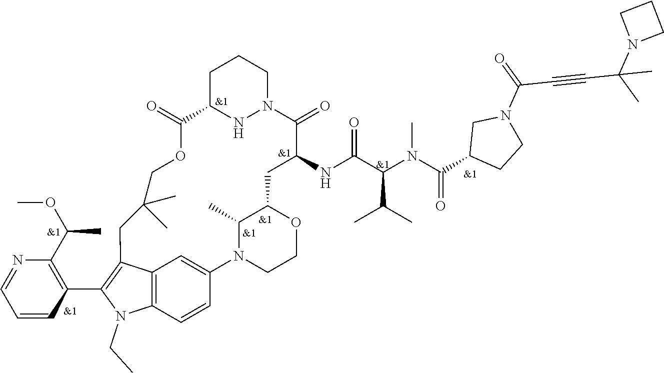 |
| BB282 | 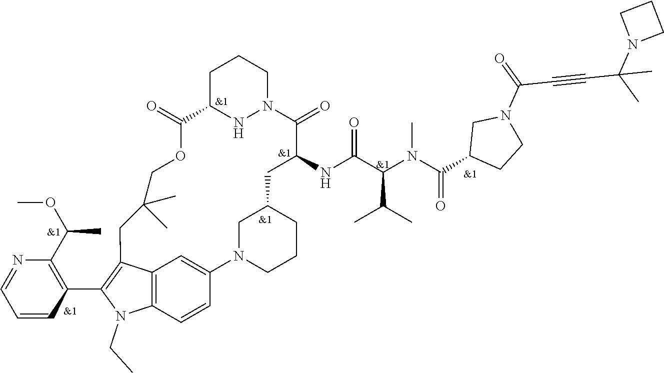 |
| BB283 | 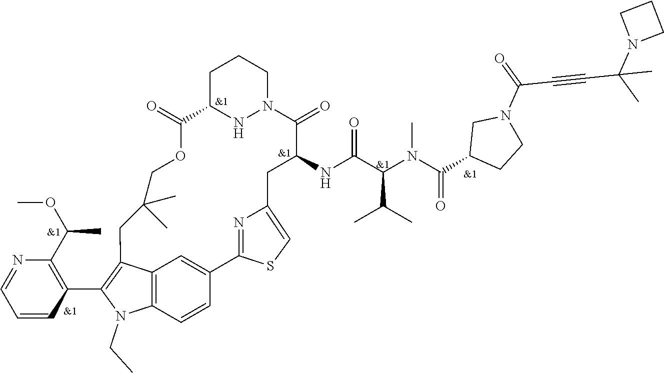 |
| BB284 | 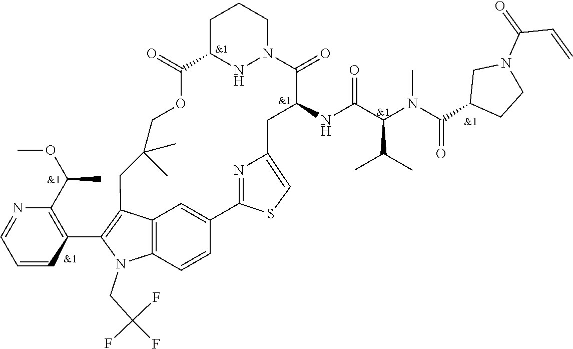 |
| BB285 | 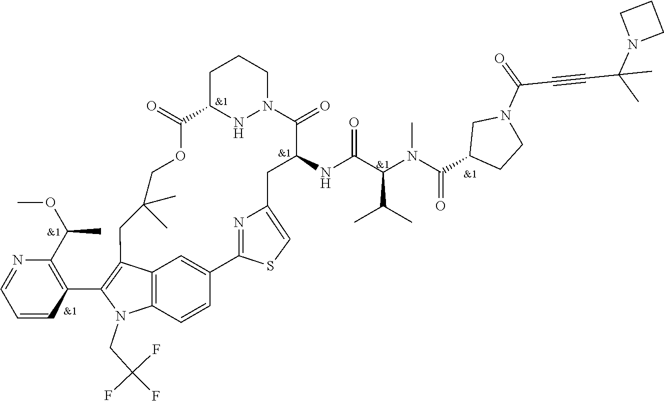 |
| BB286 | 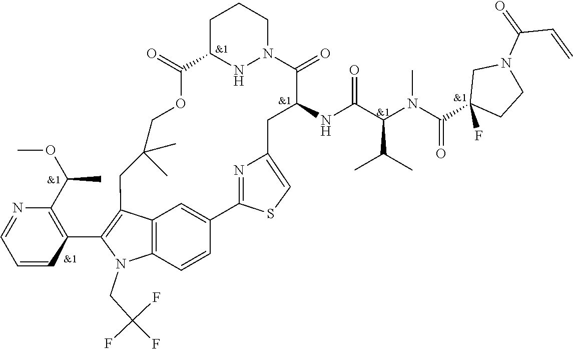 |
| BB287 | 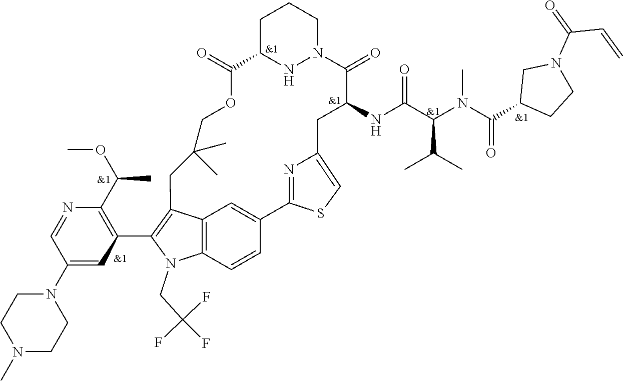 |
| BB288 | 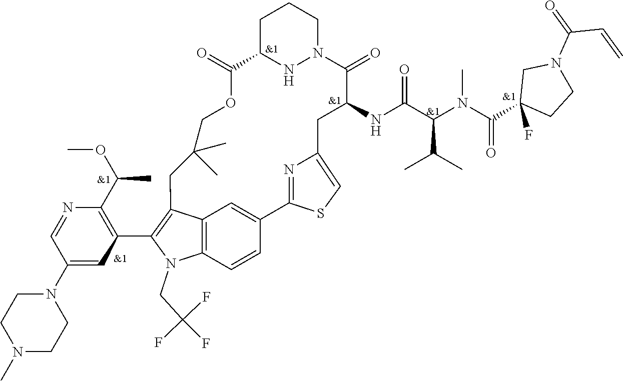 |
| BB289 | 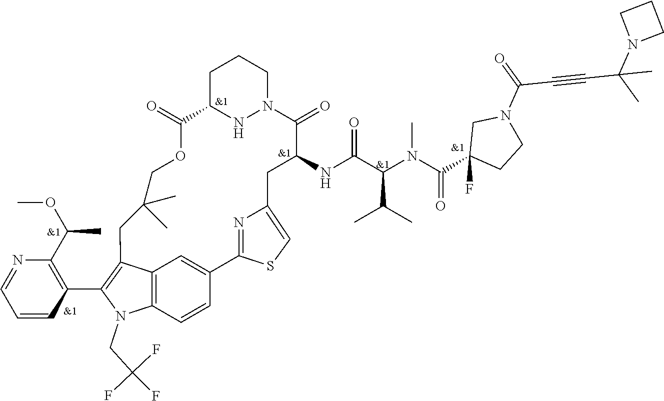 |
| BB290 | 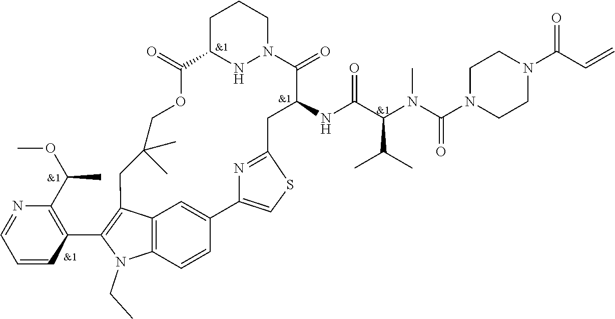 |
| BB291 | 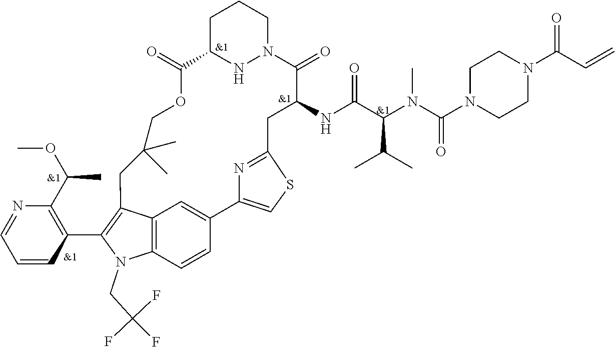 |
| BB292 | 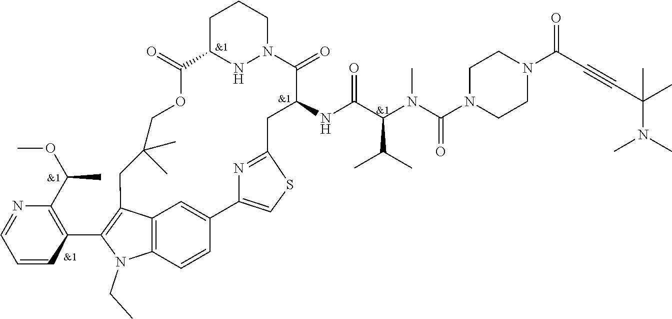 |
| BB293 | 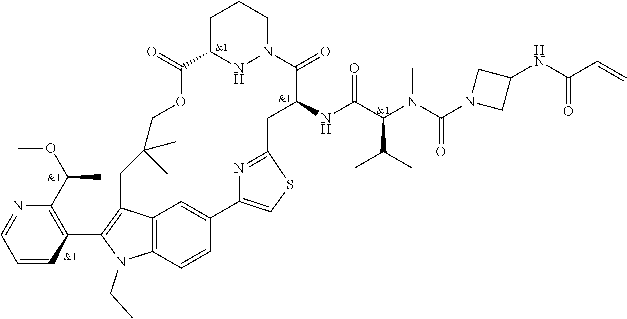 |
| BB294 | 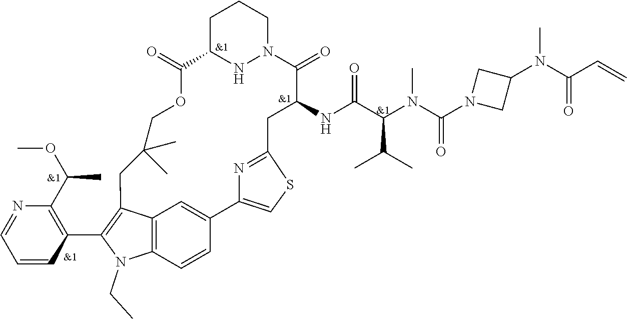 |
| BB295 | 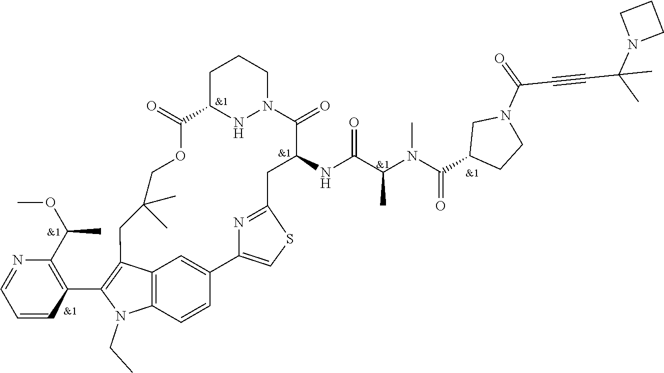 |
| BB296 | 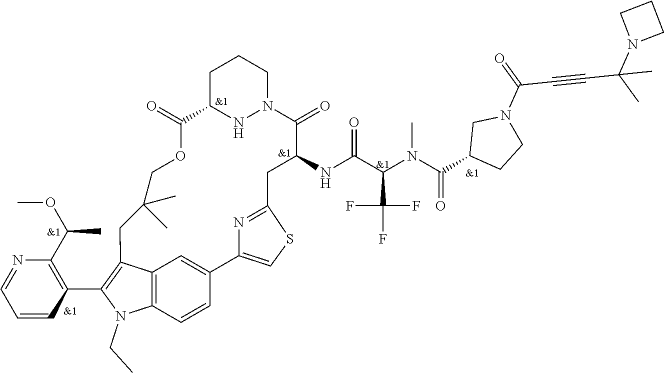 |
| BB297 | 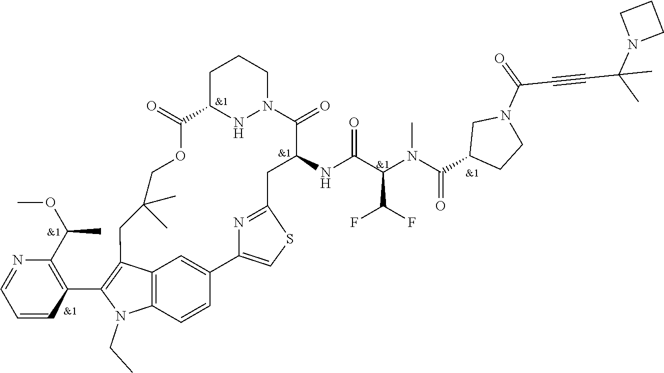 |
| BB298 | 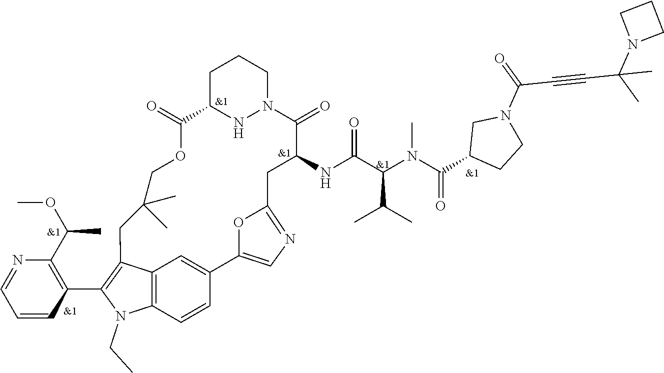 |
| BB299 | 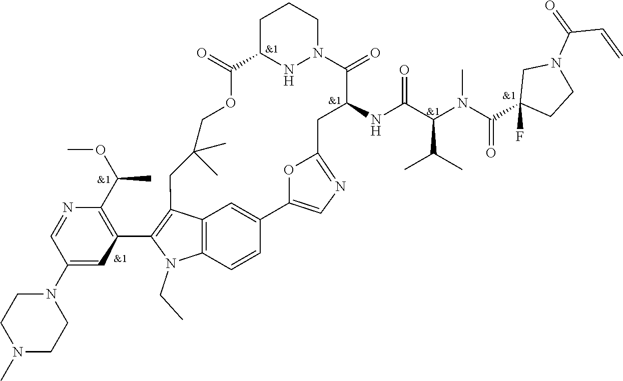 |
| BB300 | 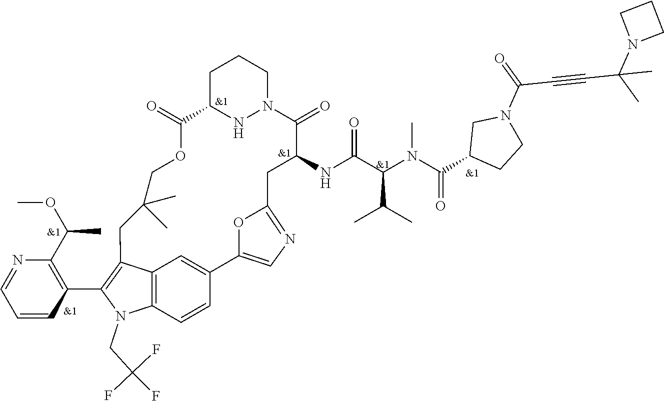 |
| BB301 | 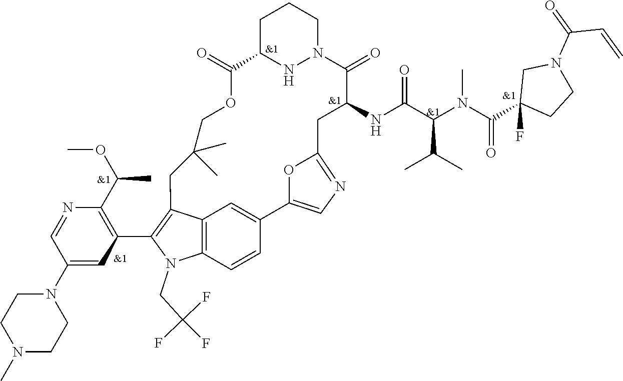 |
| BB302 | 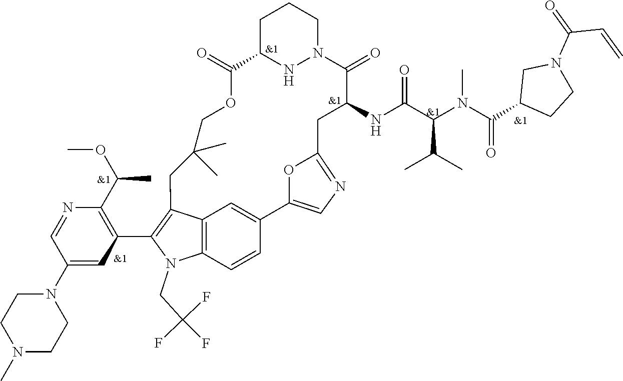 |
| BB303 | 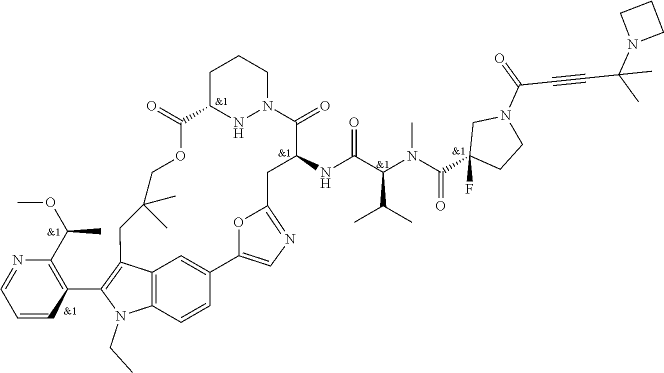 |
| BB304 | 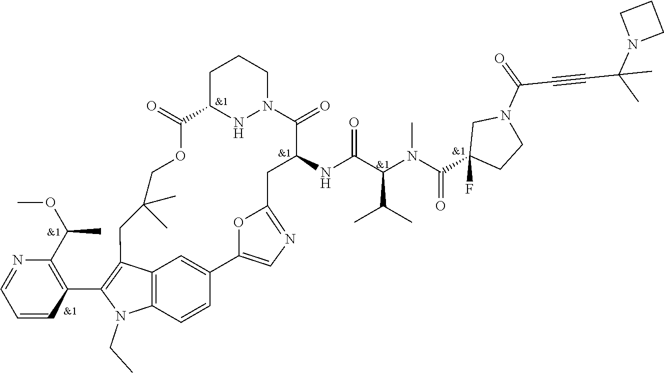 |
| BB305 | 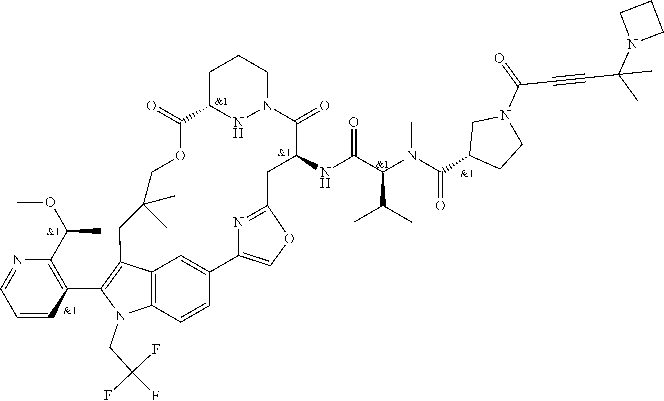 |
| BB306 | 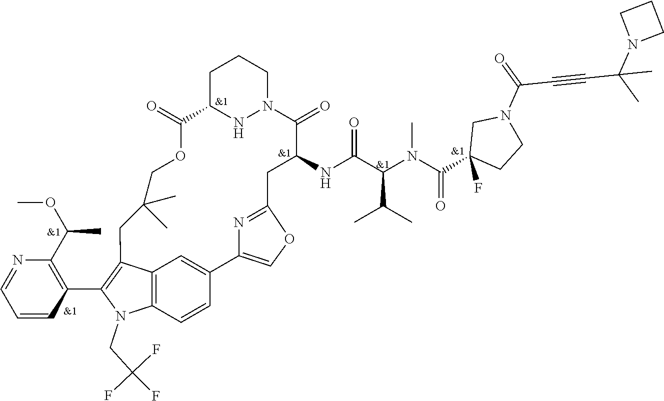 |
| BB307 | 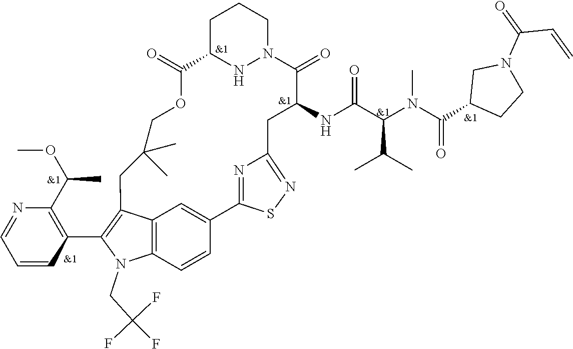 |
| BB308 | 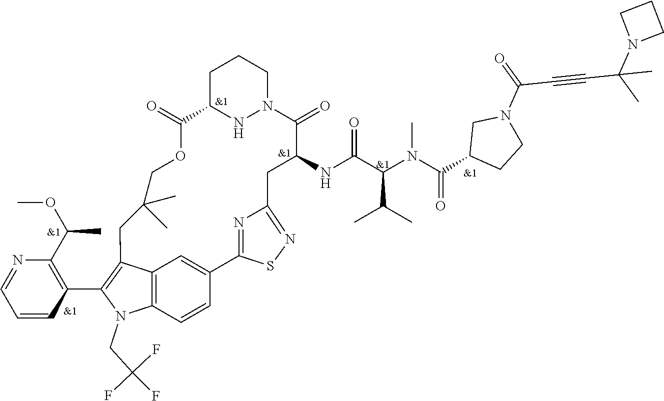 |
| BB309 | 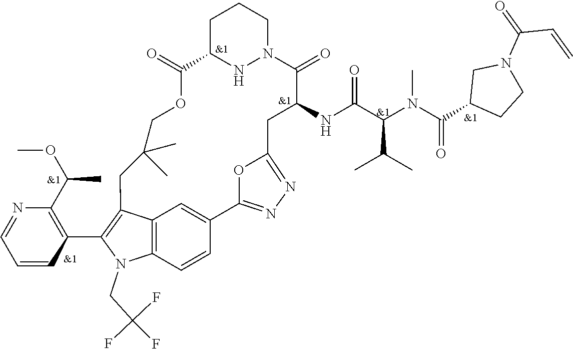 |
| BB310 | 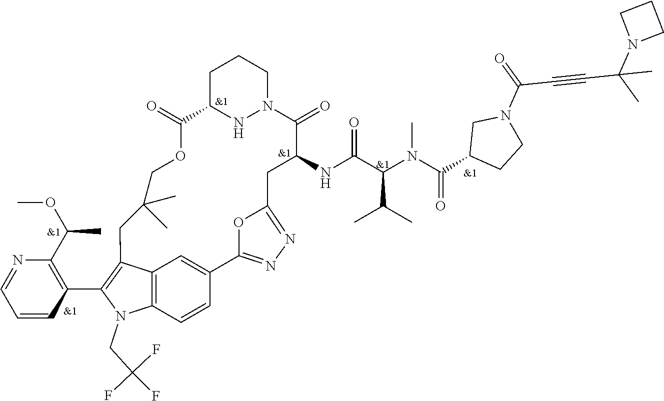 |
| BB311 | 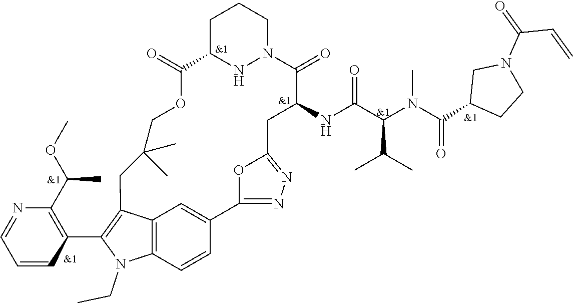 |
| BB312 | 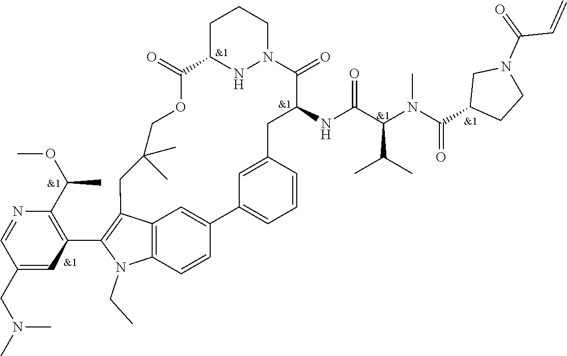 |
| BB313 | 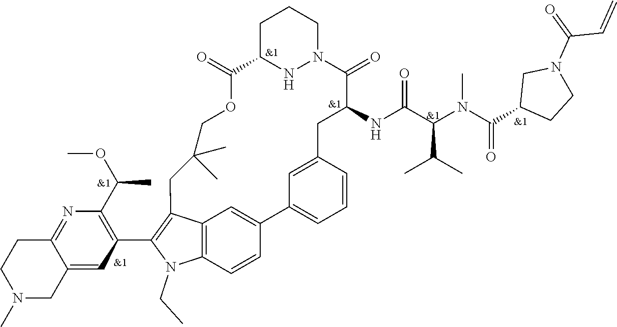 |
| BB314 | 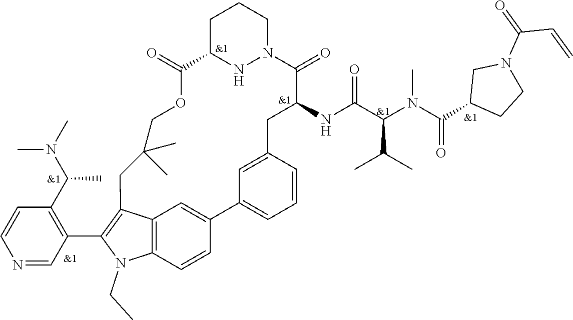 [SMILES: C=CC(=O)N1CC[C@H](C(=O)N(C)[C@H](C(=O)N[C@H]2Cc3cccc(c3)-c3ccc4c(c3)c(c(-c3cnccc3[C@@H](C)N(C)C)n4CC)CC(C)(C)COC(=O)[C@@H]3CCCN(N3)C2=O)C(C)C)C1] |
| BB315 | 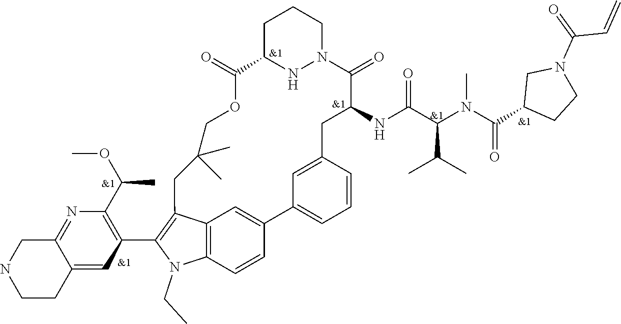 |
| BB316 | 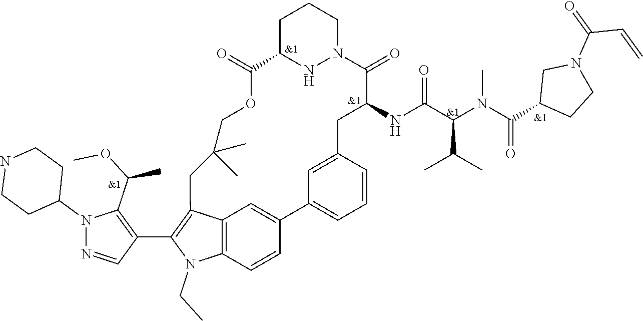 [SMILES: C=CC(=O)N1CC[C@H](C(=O)N(C)[C@H](C(=O)N[C@H]2Cc3cccc(c3)-c3ccc4c(c3)c(c(-c3cnn(C5CCNCC5)c3[C@H](C)OC)n4CC)CC(C)(C)COC(=O)[C@@H]3CCCN(N3)C2=O)C(C)C)C1] |
| BB317 | 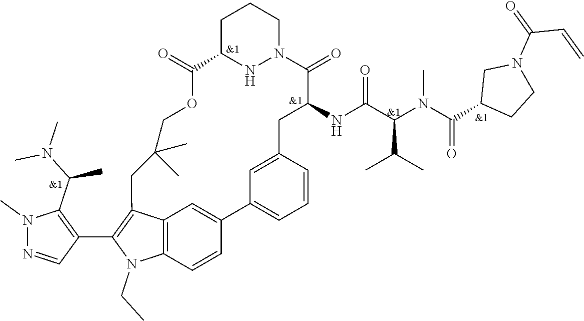 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)N(C)C)n4CC)CC(C)(C)COC(=O)[C@@H]3CCCN(N3)C2=O)C(C)C)C1] |
| BB318 | 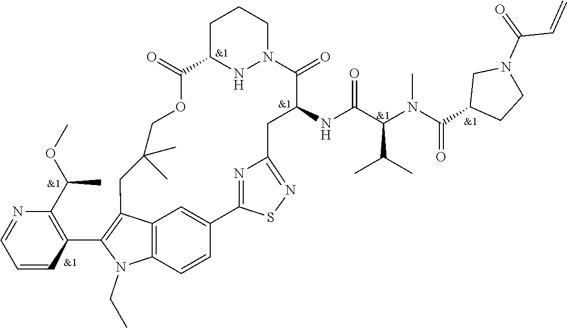 |
| BB319 | 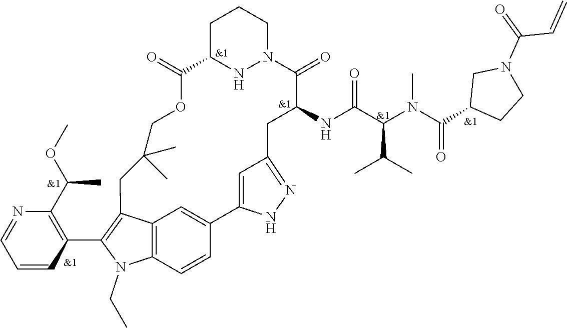 |
| BB320 | 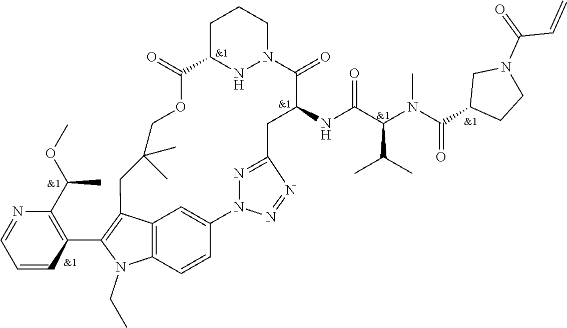 |
| BB321 | 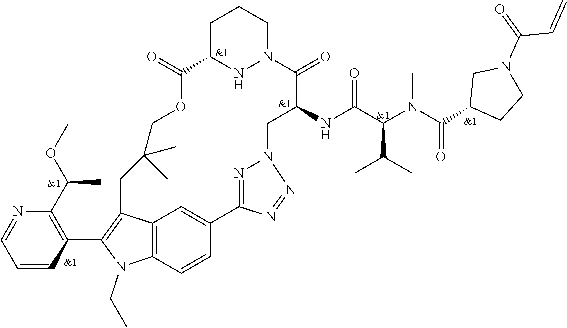 |
| BB322 | 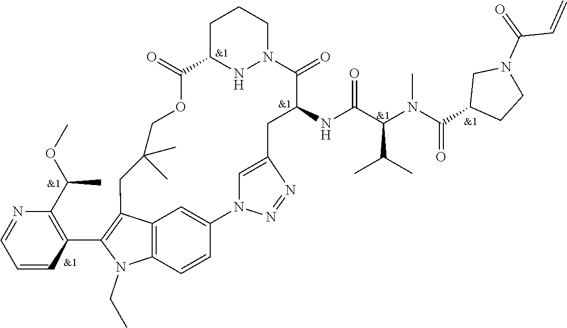 |
| BB323 | 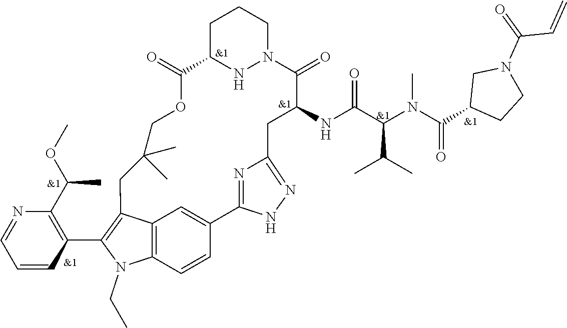 |
| BB324 | 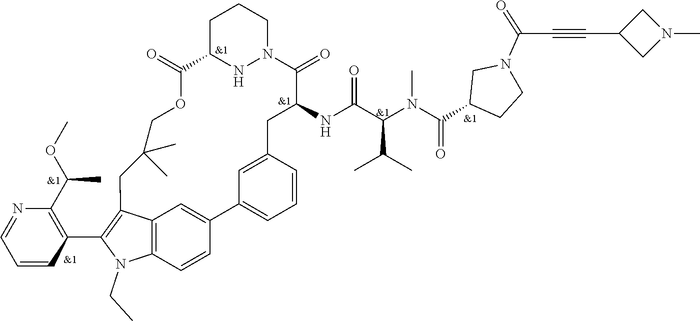 |
| BB325 | 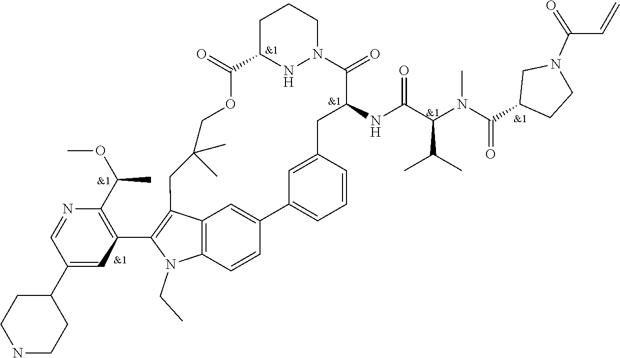 |
| BB326 | 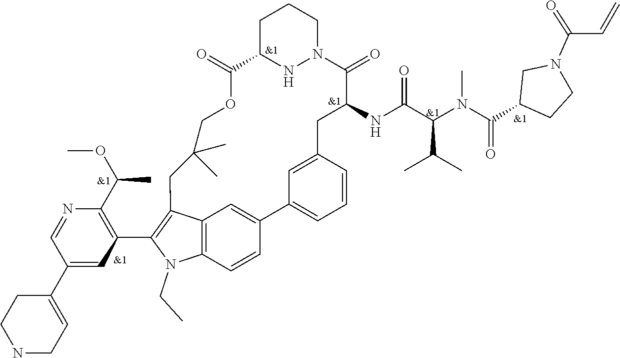 |
| BB327 | 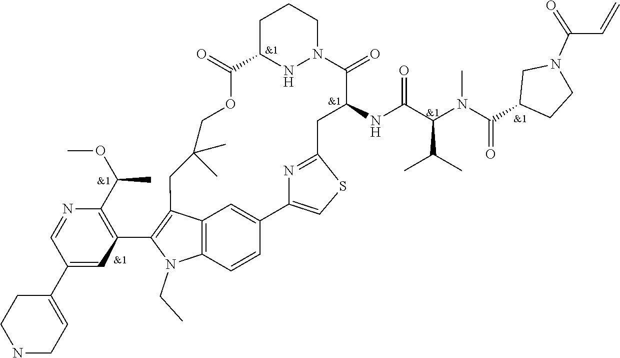 |
| BB328 | 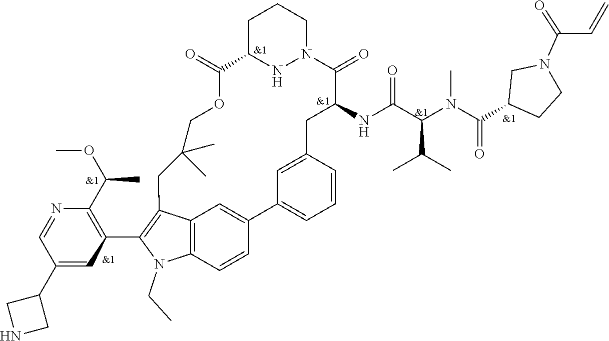 |
| BB329 | 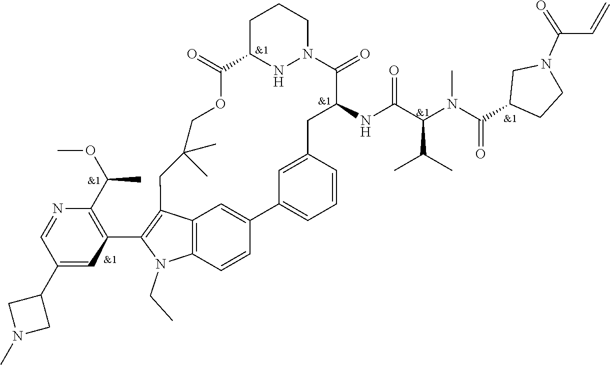 |
| BB330 | 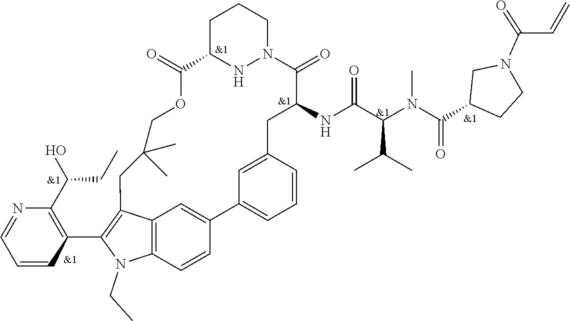 |
| BB331 | 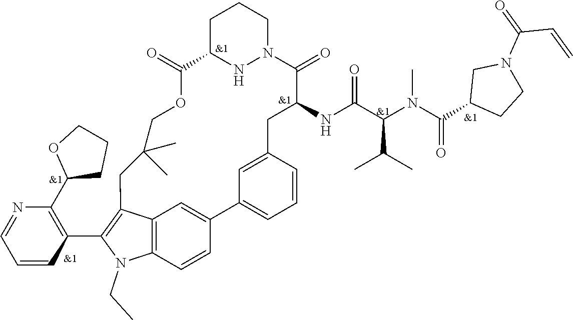 |
| BB332 | 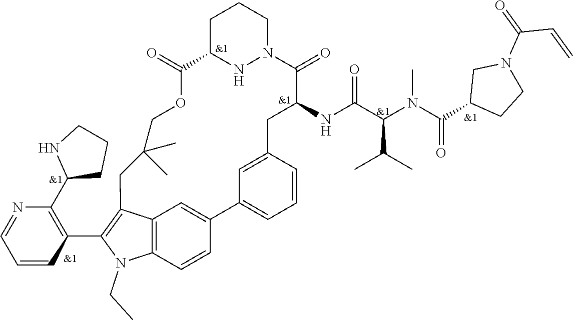 |
| BB333 | 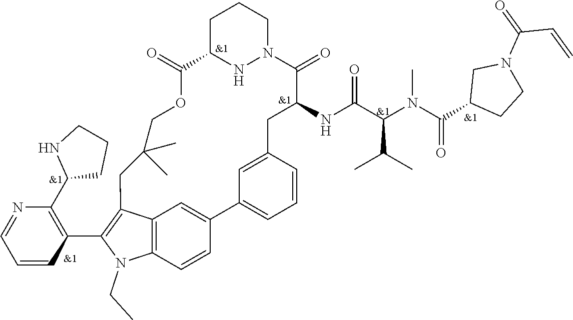 |
| BB334 | 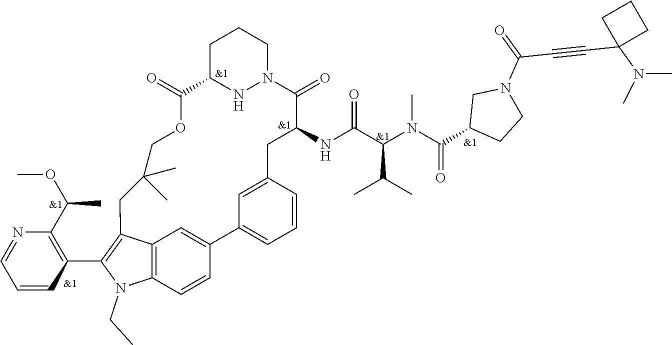 |
| BB335 | 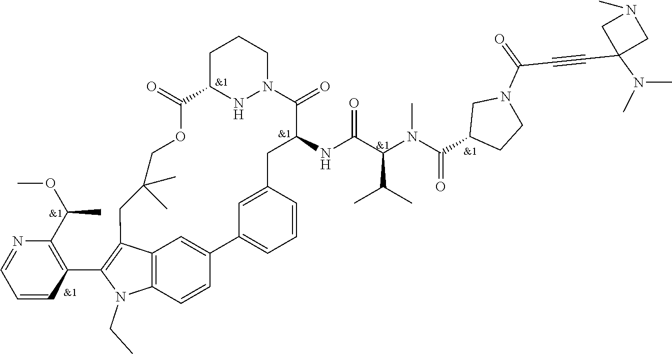 |
| BB336 | 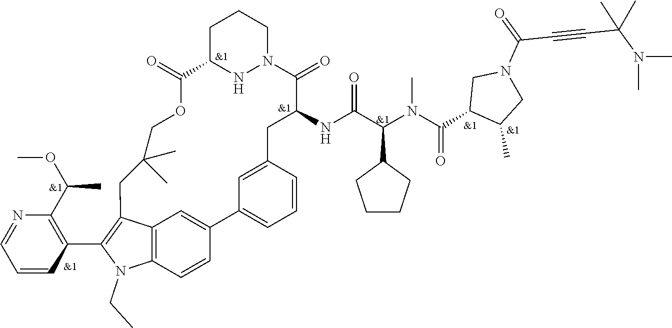 |
| BB337 | 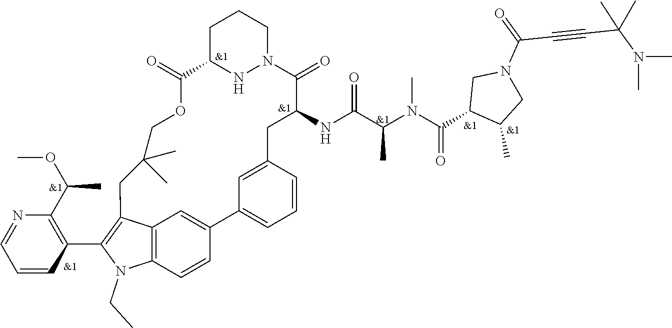 |
| BB338 | 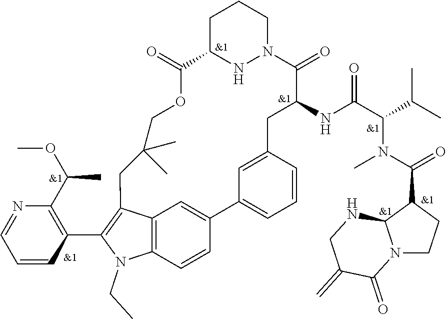 |
| BB339 | 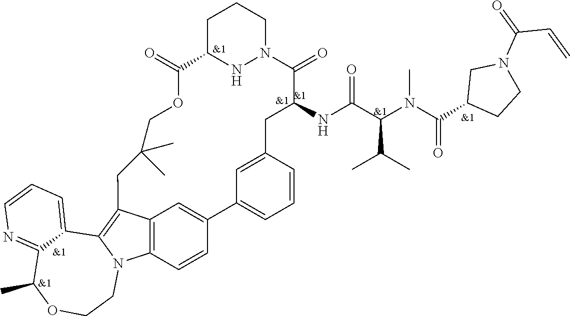 |
| BB340 | 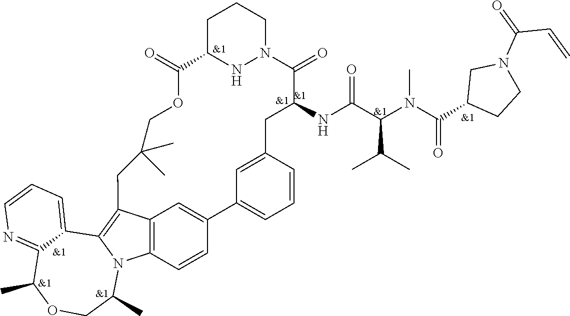 |
| BB341 | 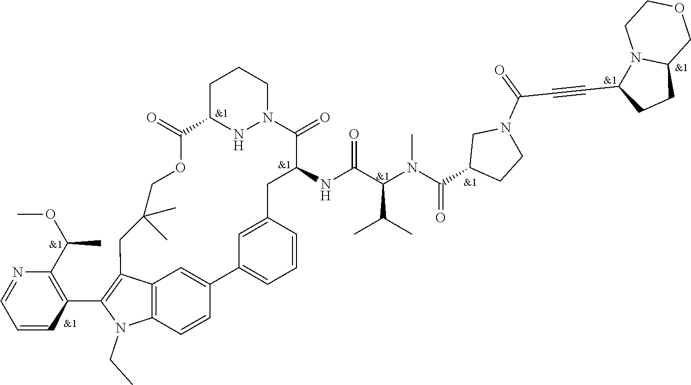 |
| BB342 | 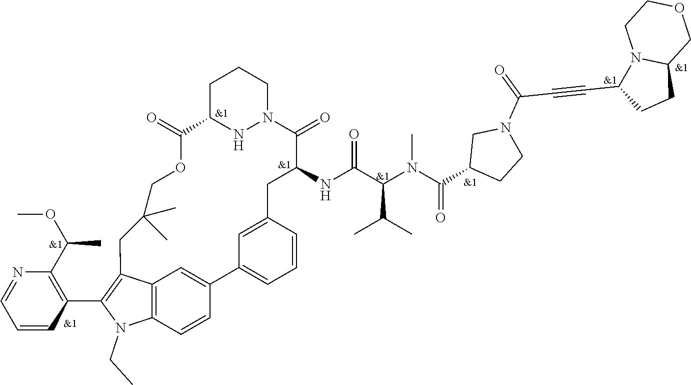 |
| BB343 | 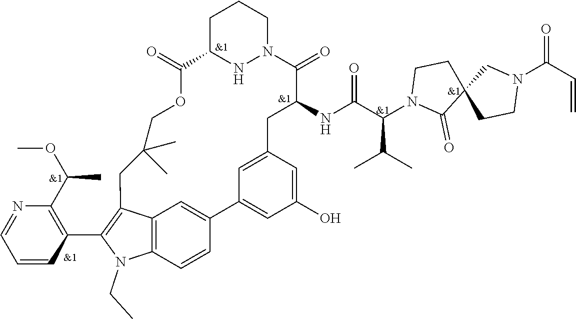 |
| BB344 | 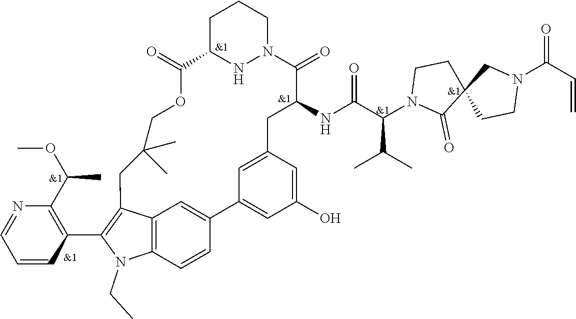 |
| BB345 | 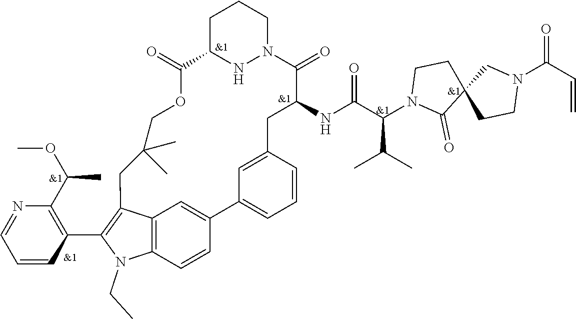 |
| BB346 | 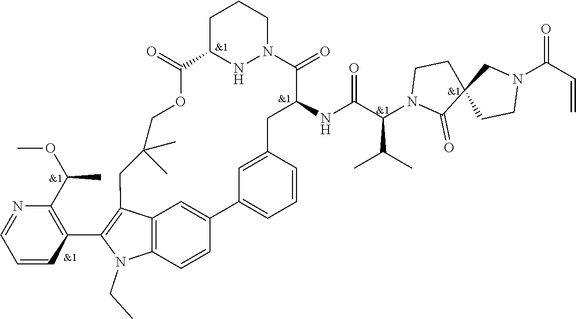 |
| BB347 | 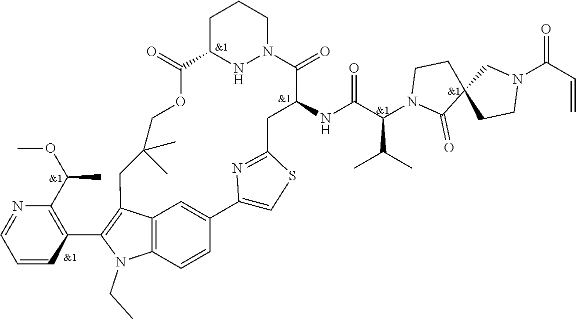 |
| BB348 | 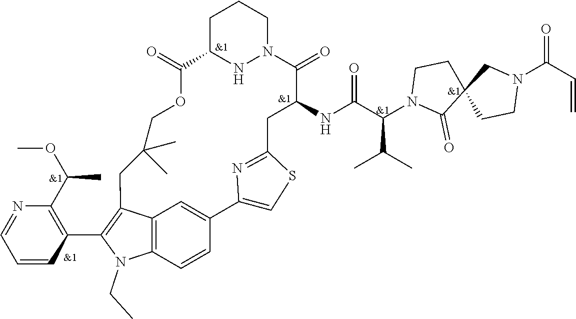 |
| BB349 | 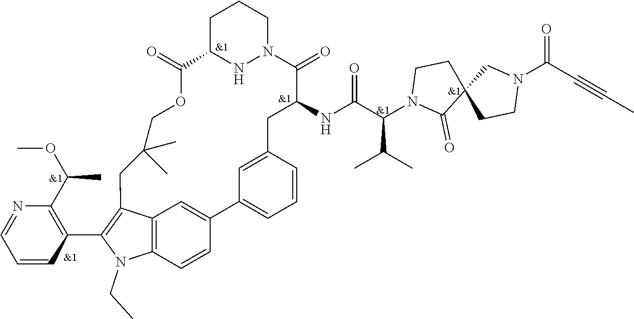 |
| BB350 | 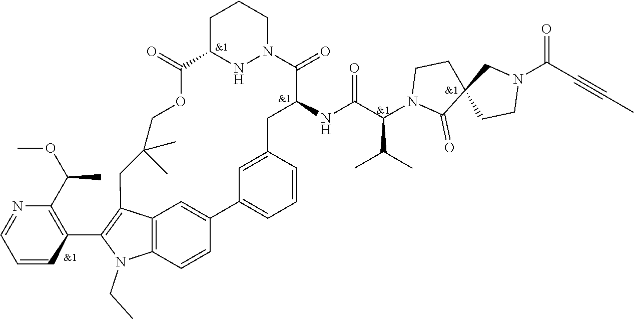 |
| BB351 | 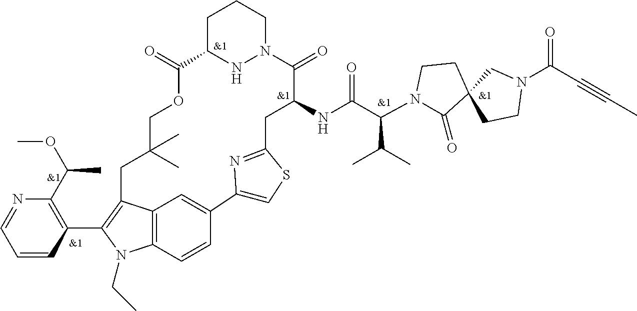 |
| BB352 | 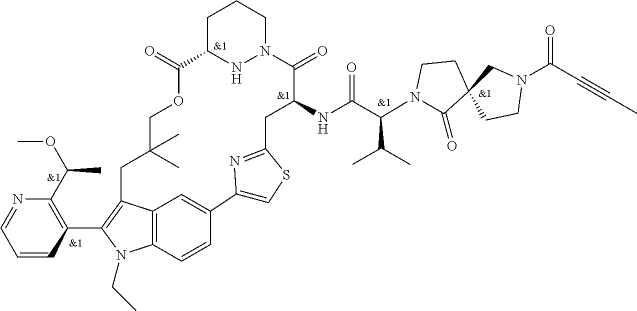 |
| BB353 | 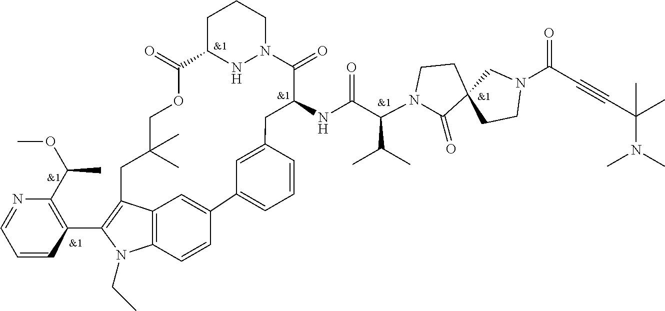 |
| BB354 | 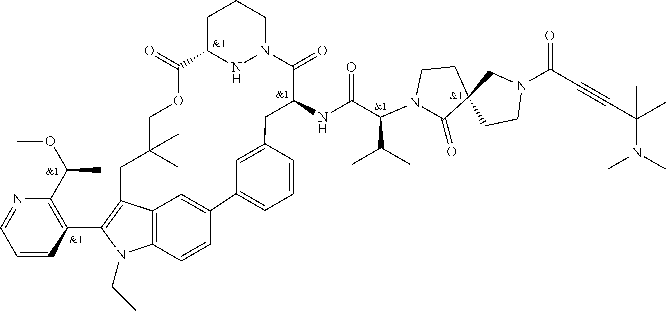 |
| BB355 | 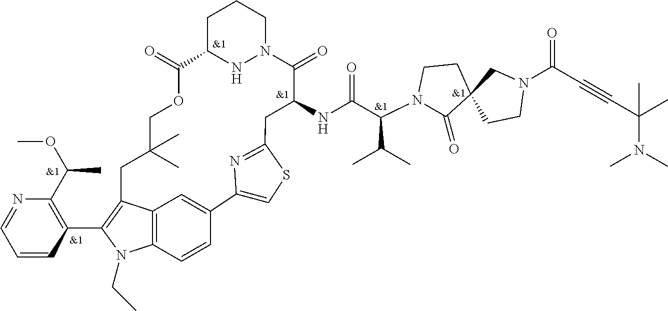 |
| BB356 | 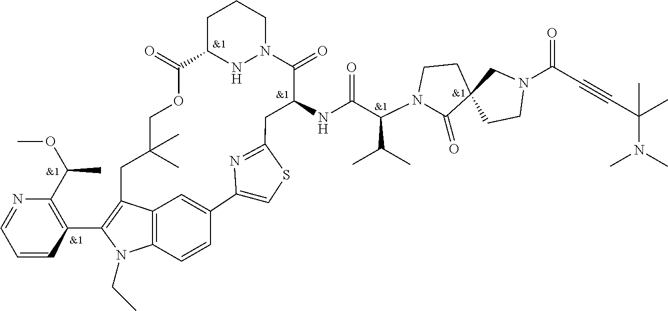 |
| BB357 | 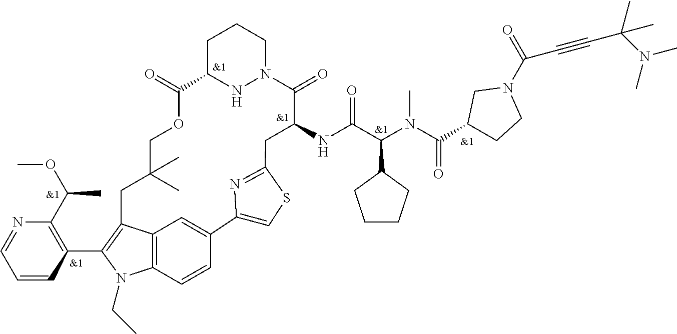 |
| BB358 | 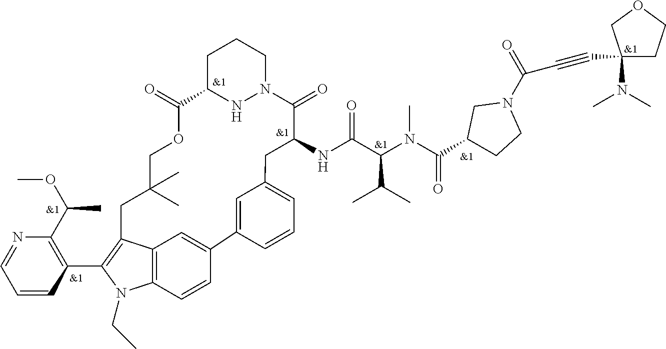 |
| BB359 | 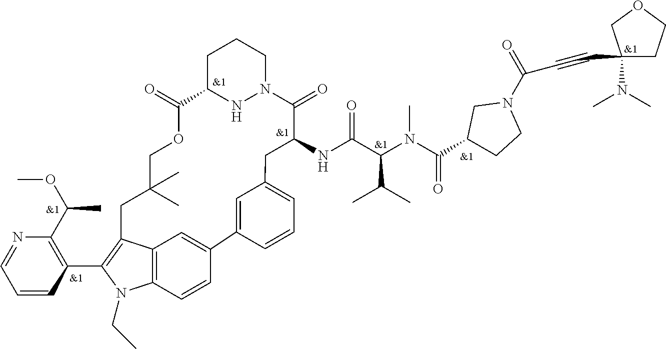 |
| BB360 | 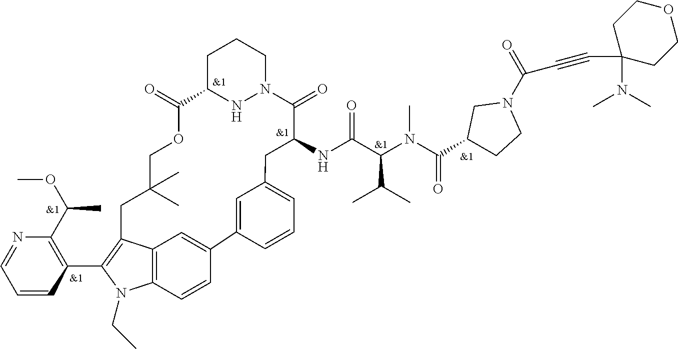 |
| BB361 | 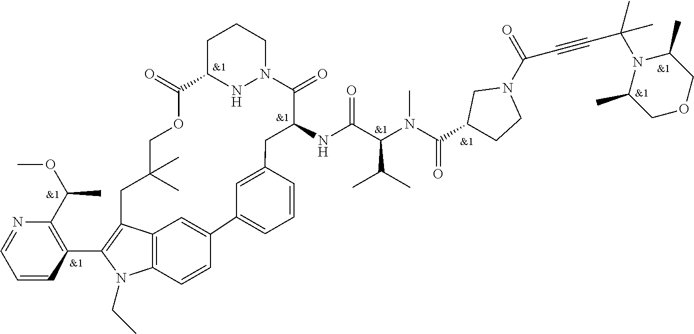 |
| BB362 | 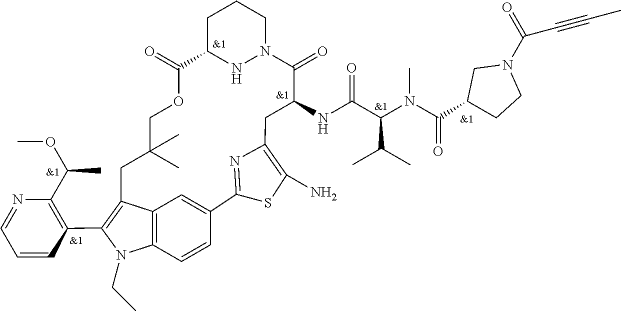 |
| BB363 | 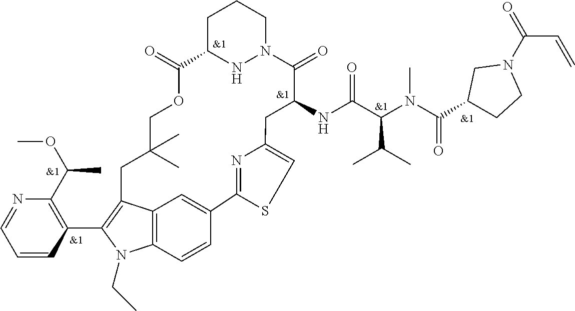 |
| BB364 | 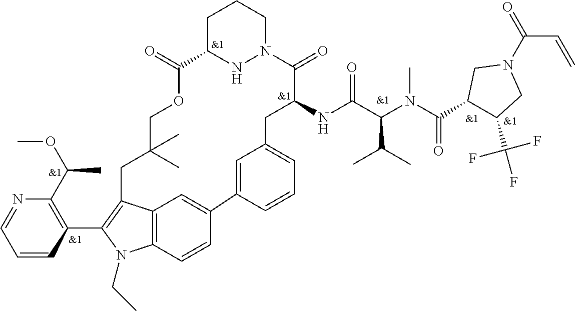 |
| BB365 | 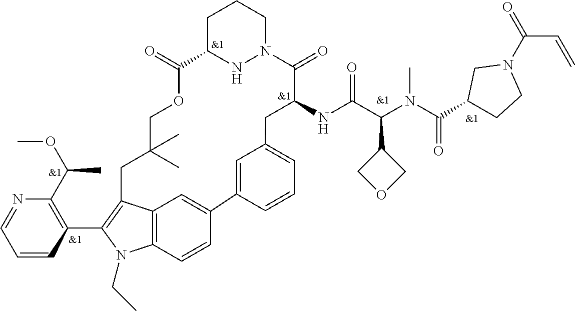 |
| BB366 | 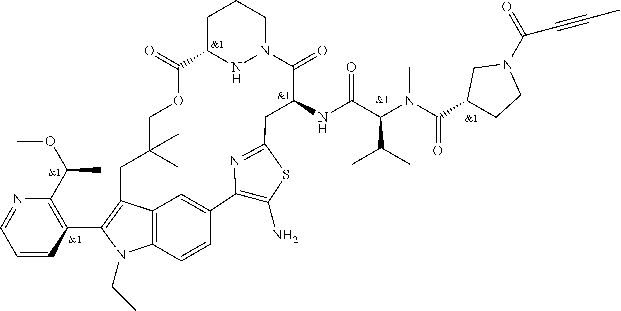 |
| BB367 | 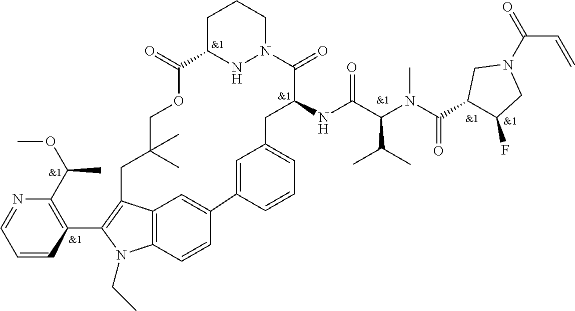 |
| BB368 | 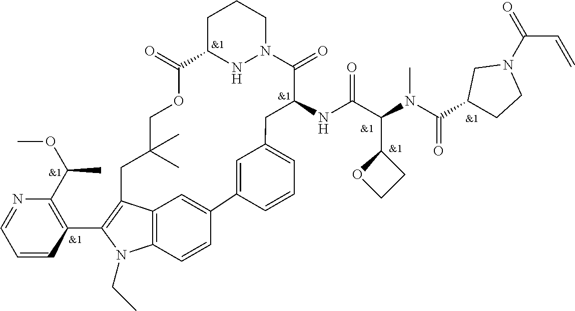 |
| BB369 | 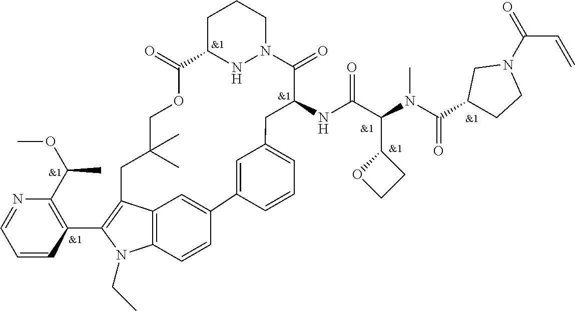 |
| BB370 | 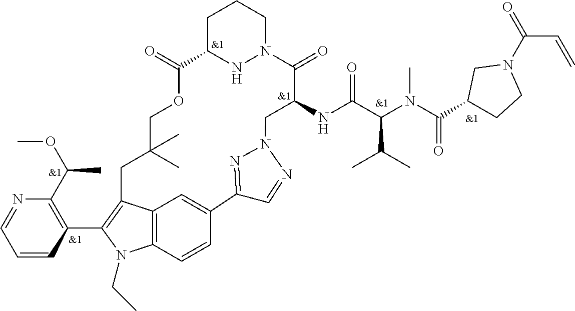 |
| BB371 | 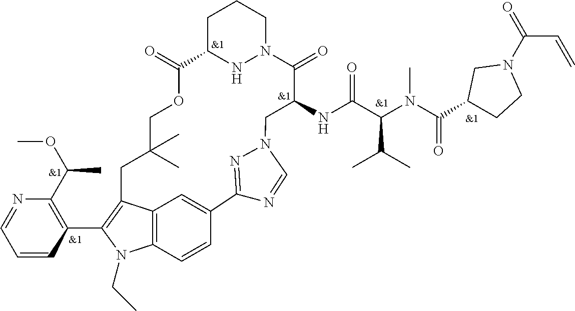 |
| BB372 | 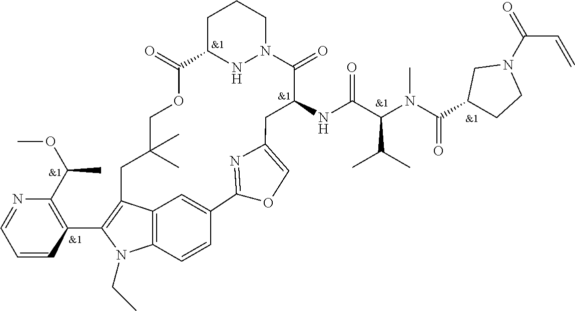 |
| BB373 | 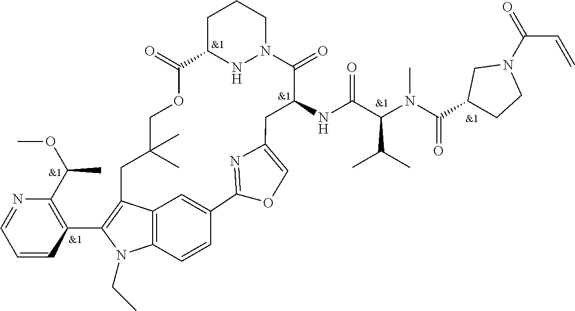 |
| BB374 | 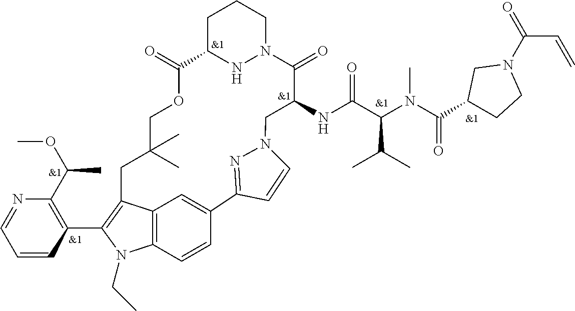 |
| BB375 | 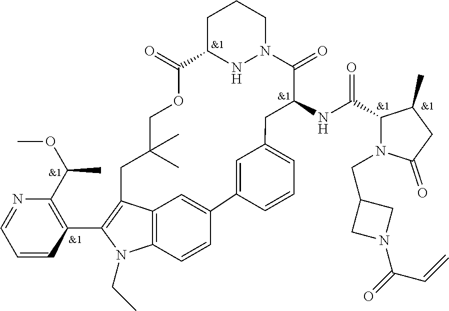 |
| BB376 | 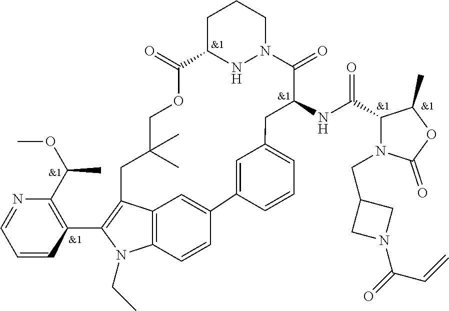 |
| BB377 | 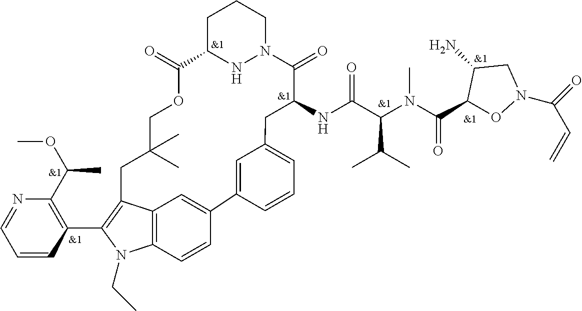 |
| BB378 | 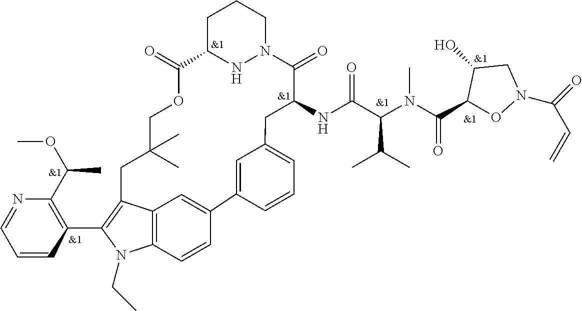 |
| BB379 | 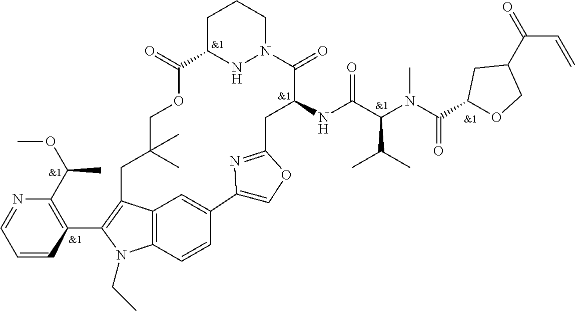 |
| BB380 | 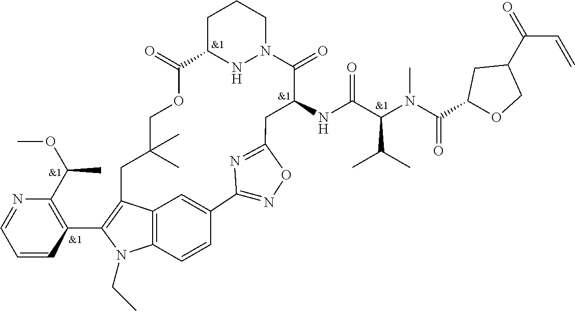 |
| BB381 | 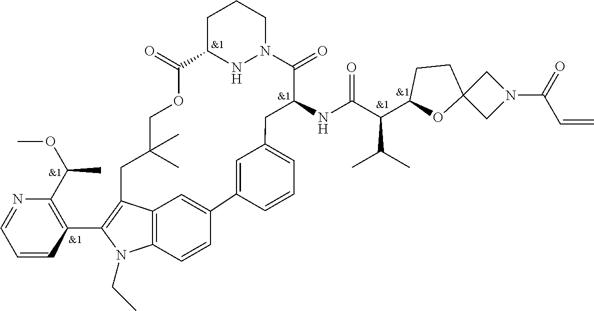 |
| BB382 | 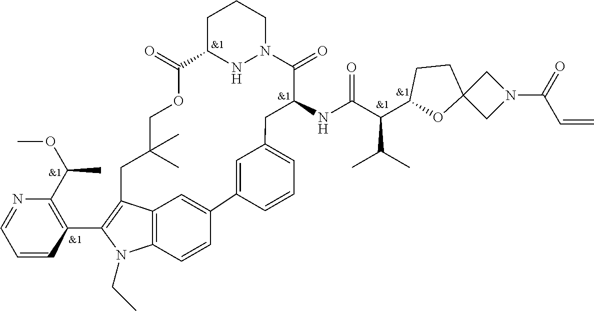 |
| BB383 | 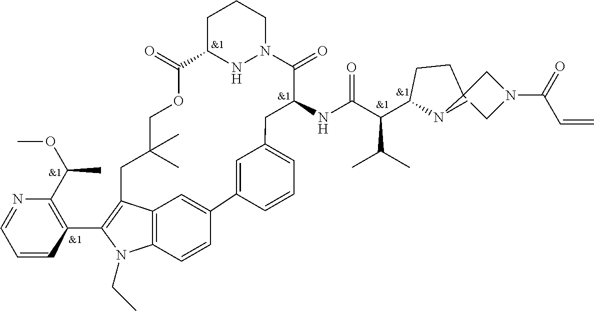 |
| BB384 | 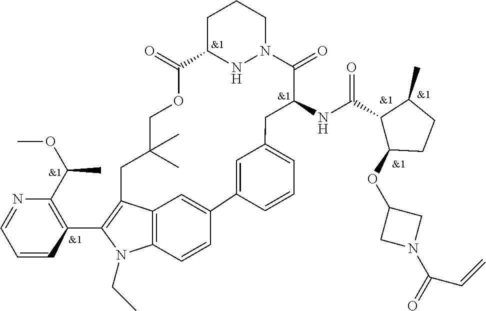 |
| BB385 | 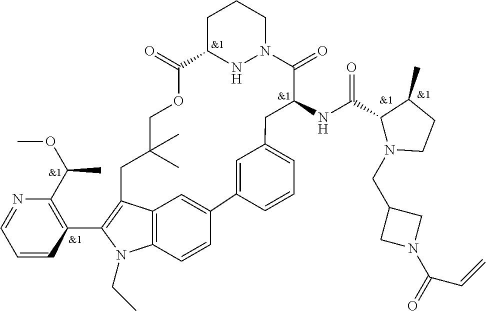 |
| BB386 | 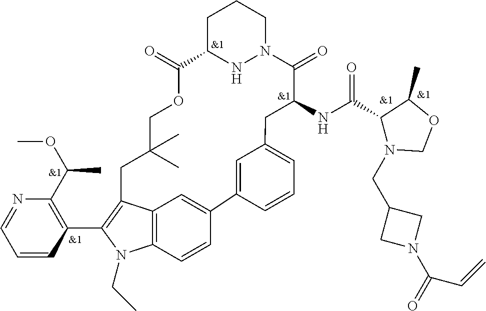 |
| BB387 | 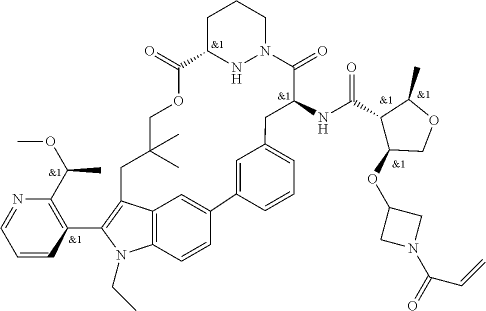 |
| BB388 | 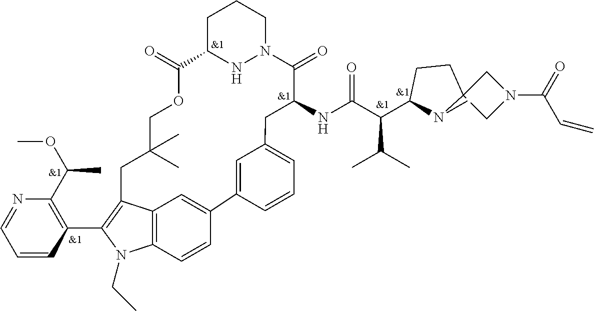 |
| BB389 | 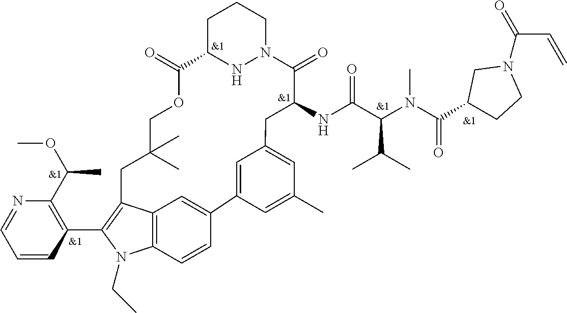 |
| BB390 | 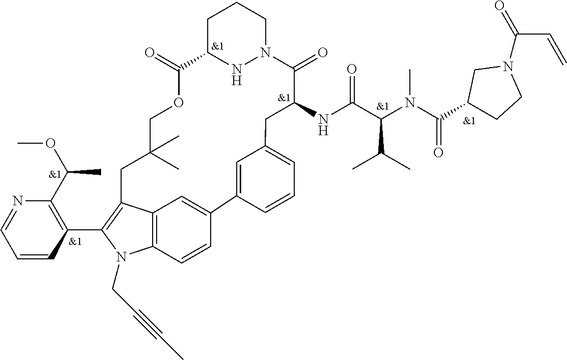 |
| BB391 | 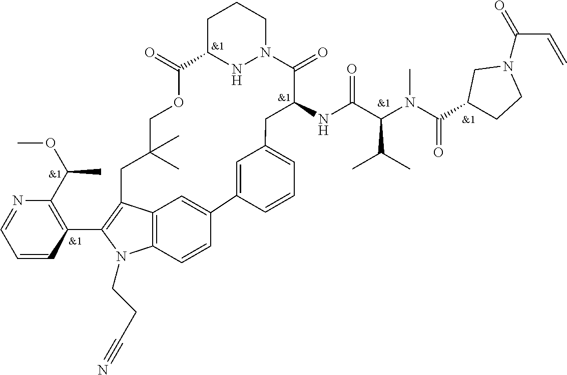 |
| BB392 | 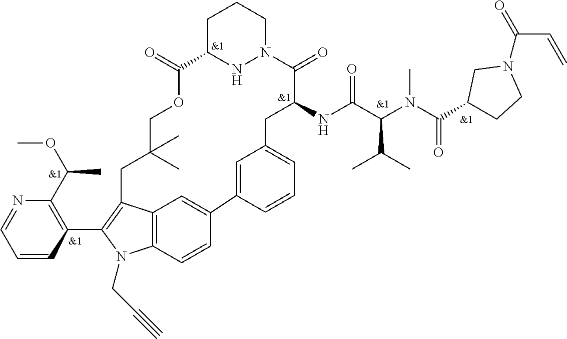 |
| BB393 | 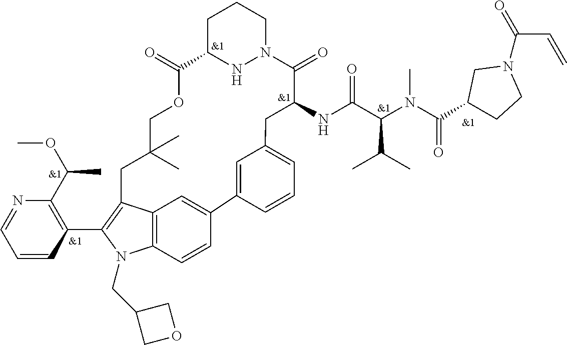 |
| BB394 | 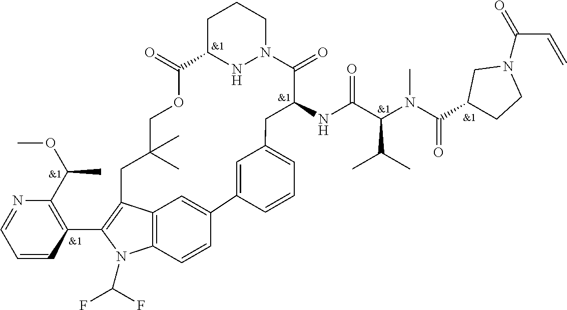 |
| BB395 | 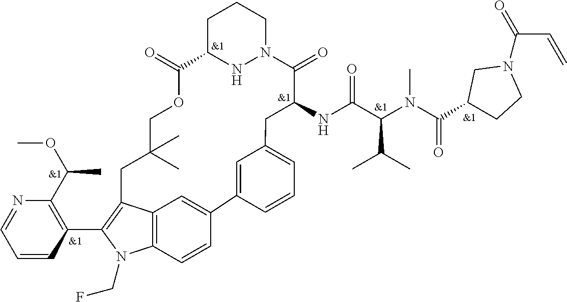 |
| BB396 | 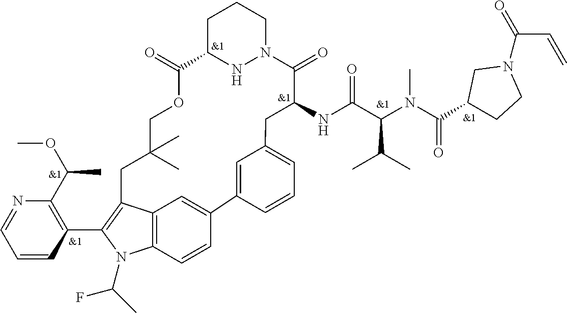 |
| BB397 | 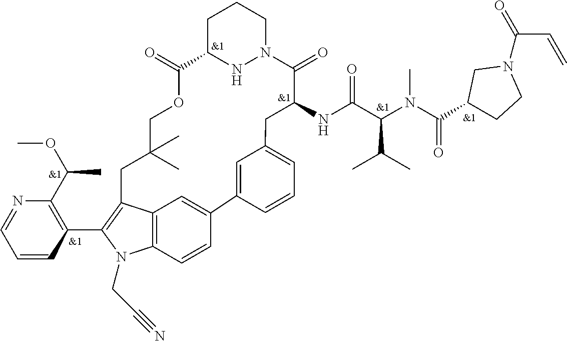 |
| BB398 | 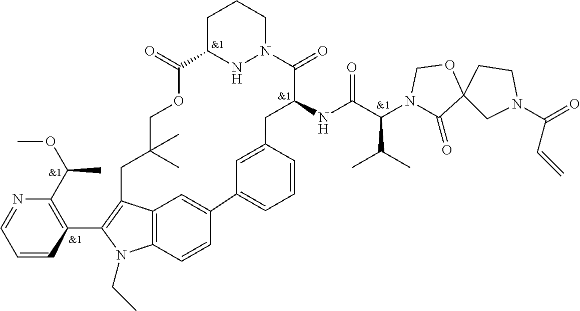 |
| BB399 | 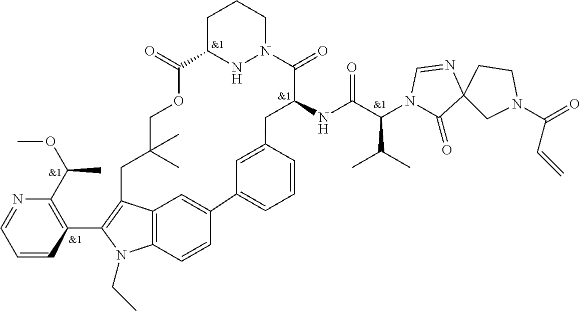 |
| BB400 | 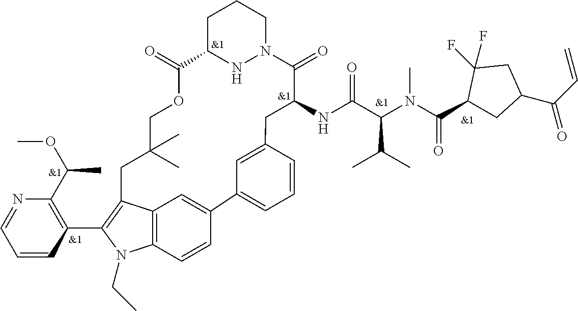 |
| BB401 | 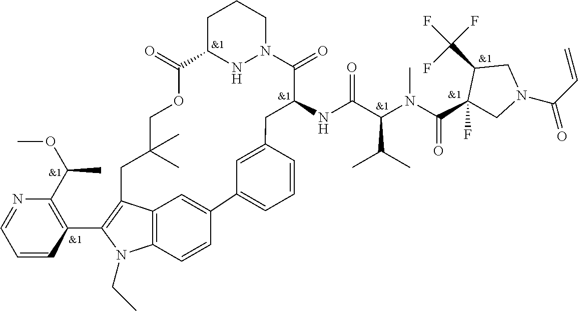 |
| BB402 | 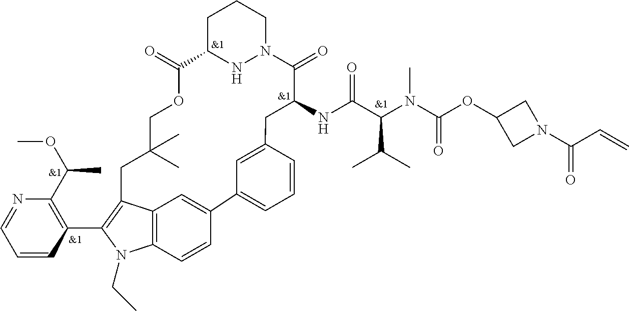 |
| BB403 | 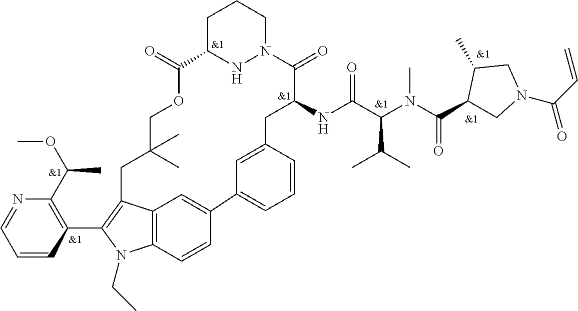 |
| BB404 | 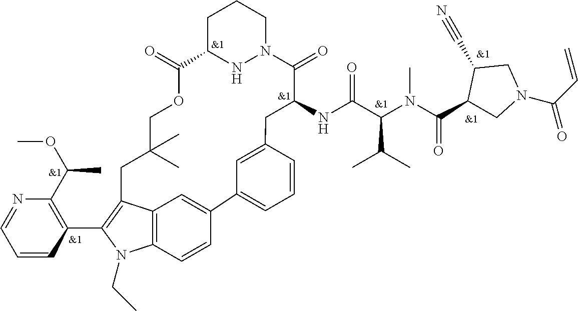 |
| BB405 | 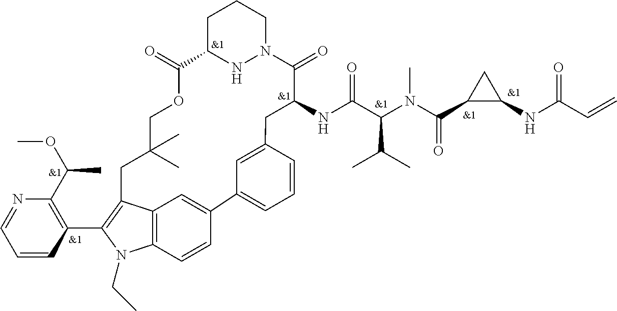 |
| BB406 | 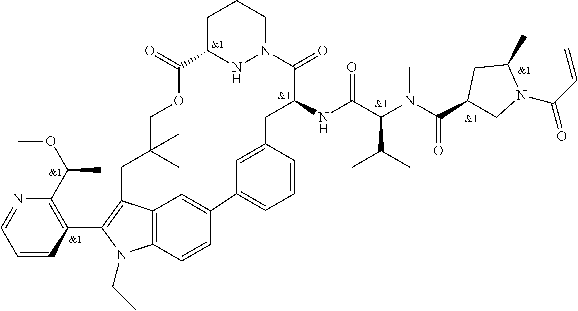 |
| BB407 | 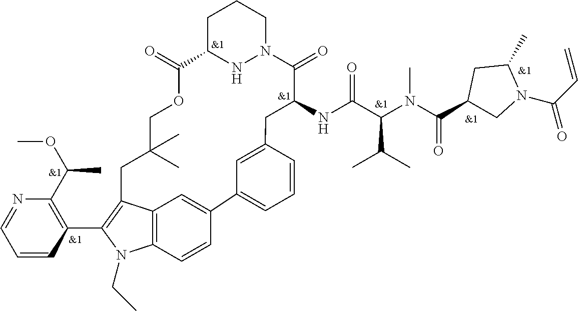 |
| BB408 | 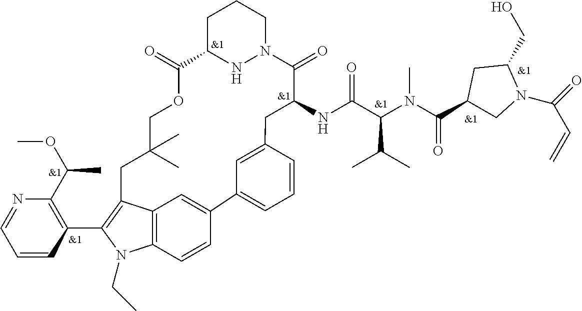 |
| BB409 | 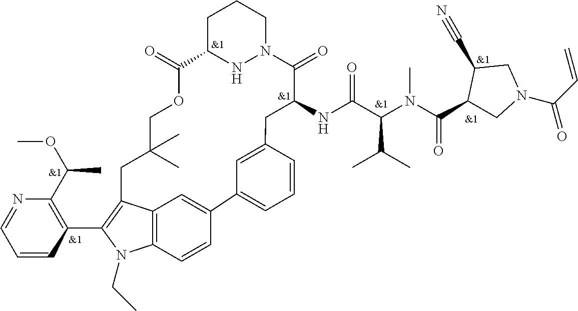 |
| BB410 | 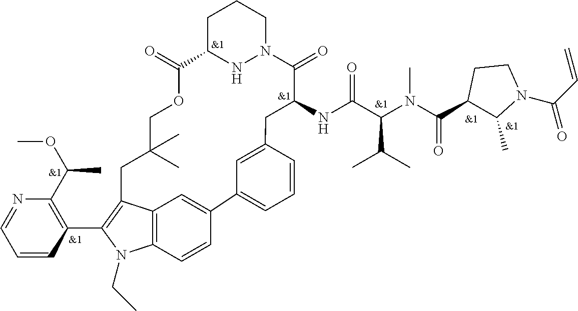 |
| BB411 | 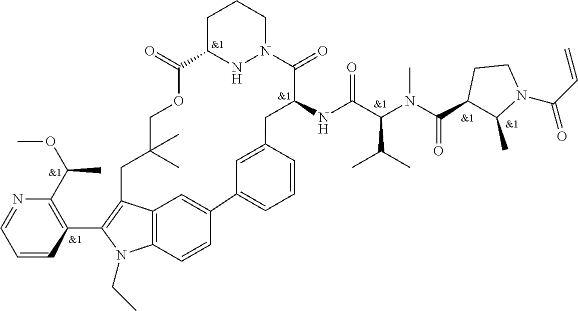 |
| BB412 | 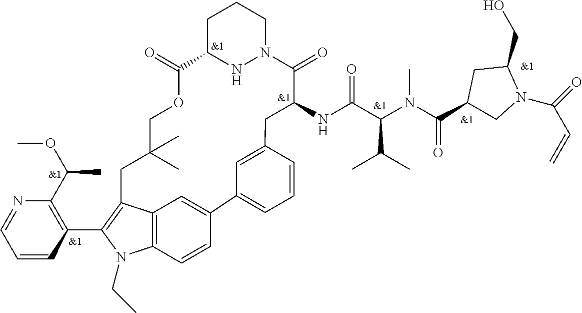 |
| BB413 | 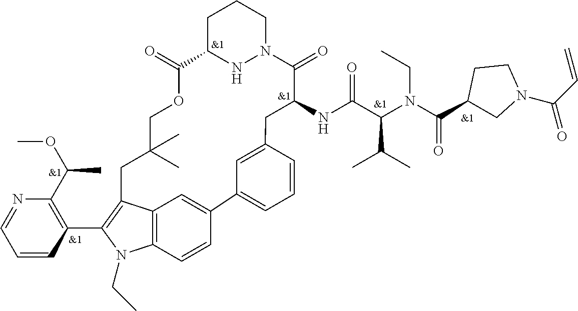 |
| BB414 | 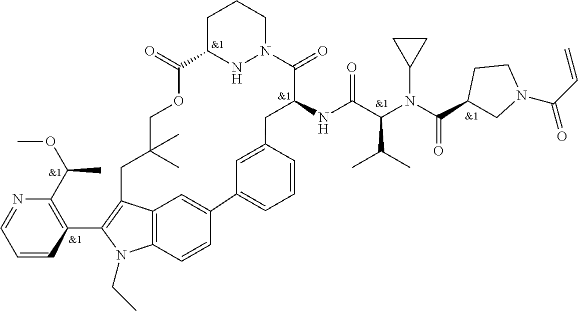 |
| BB415 | 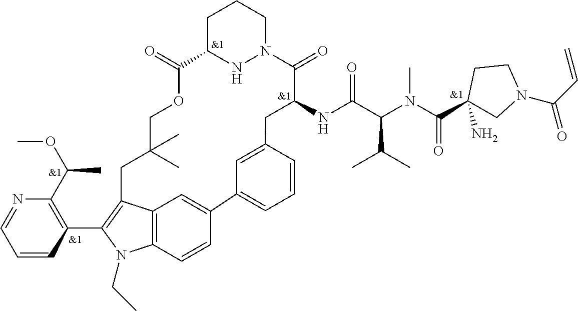 |
| BB416 | 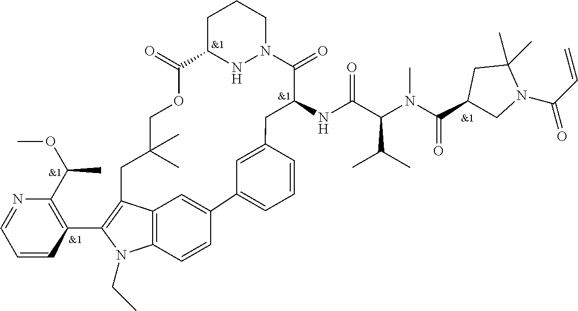 |
| BB417 | 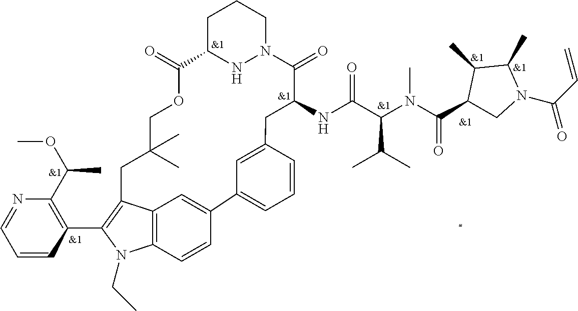 |
| BB418 | 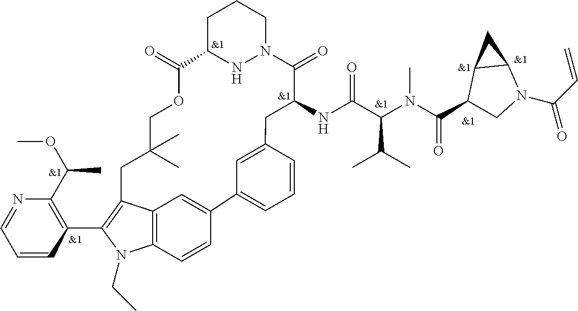 |
| BB419 | 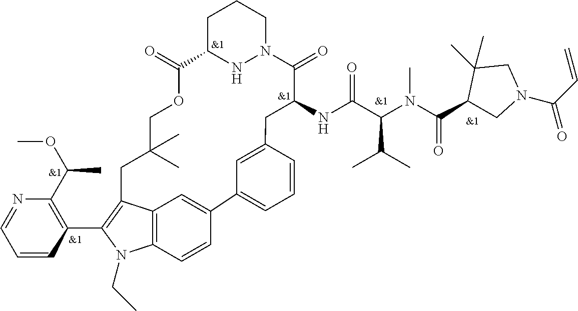 |
| BB420 | 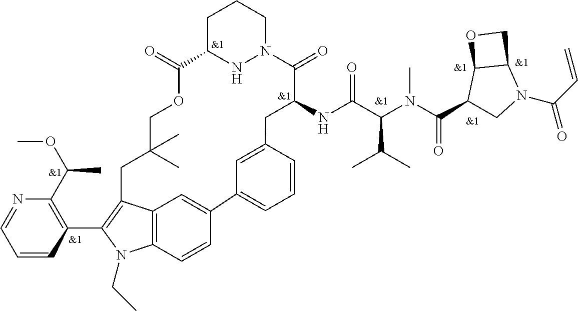 |
| BB421 | 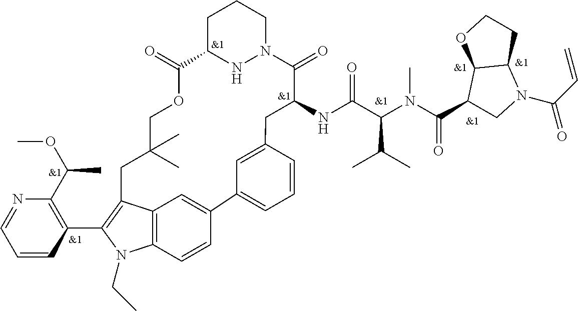 |
| BB422 | 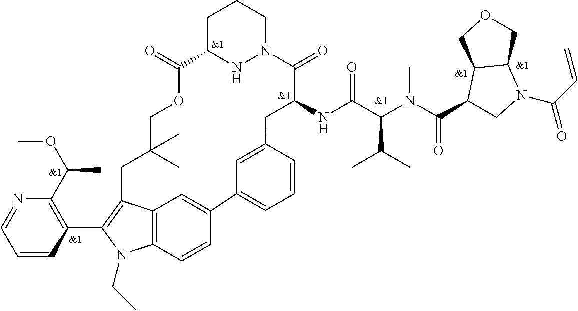 |
| BB423 | 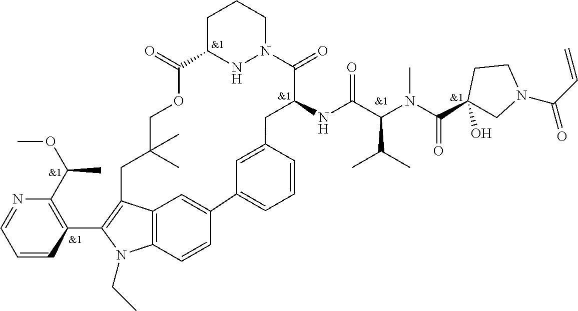 |
| BB424 | 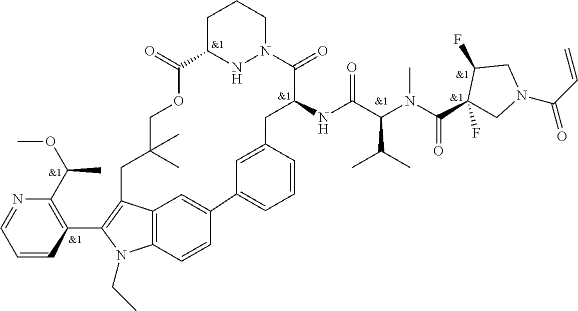 |
| BB425 | 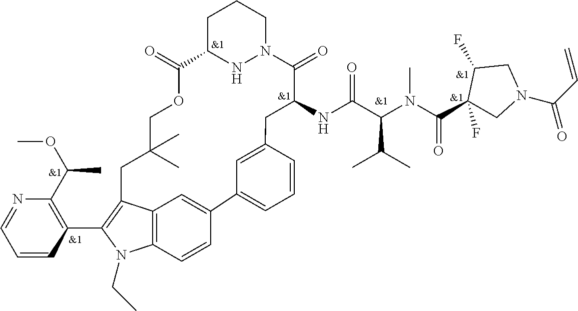 |

Note
that some compounds are shown with bonds as flat or wedged. In some instances, the relative stereochemistry of stereoisomers has been
determined; in some instances, the absolute stereochemistry has been determined. All stereoisomers of the compounds of the foregoing table
are contemplated by the present invention. In particular embodiments, an atropisomer of a compound of the foregoing table is contemplated.

In some embodiments, the RAS(ON) inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

The compounds described in Tables B1 and B2 may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below or as described in WO 2021/091982.

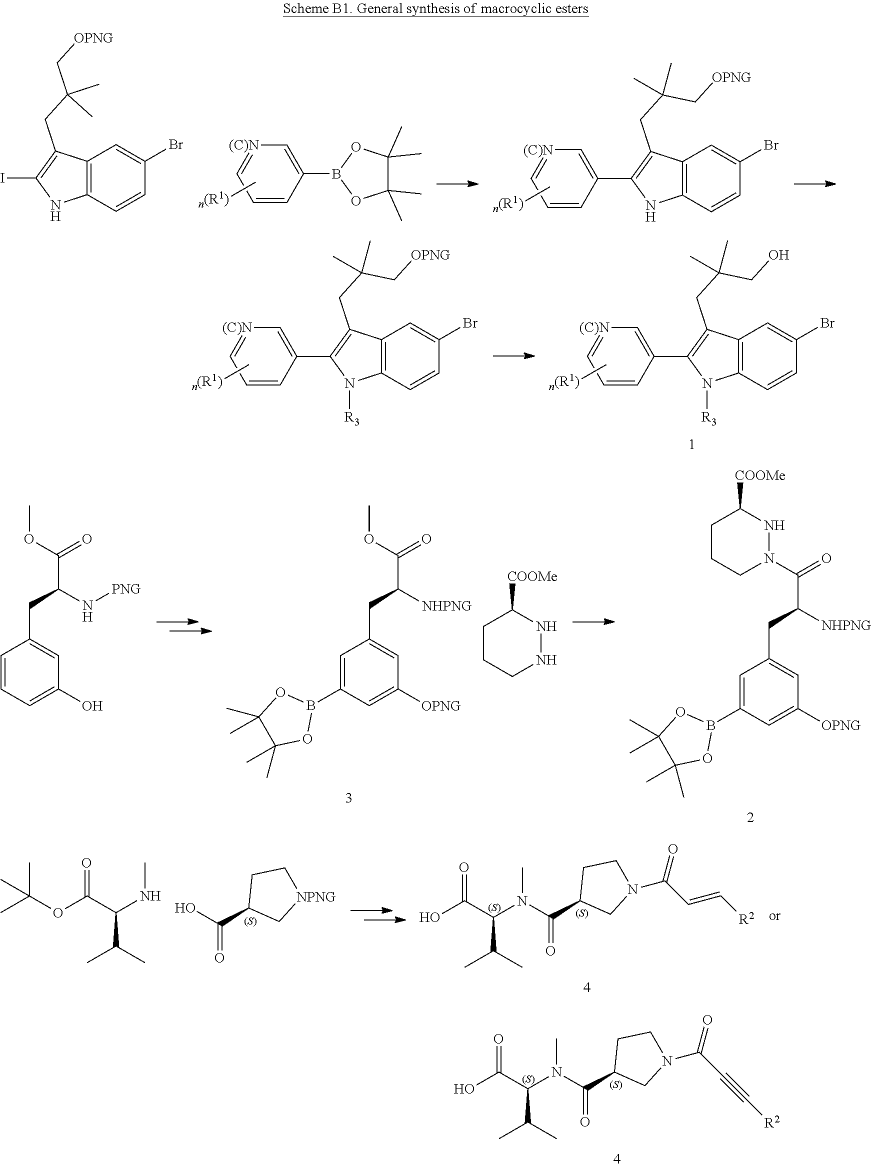

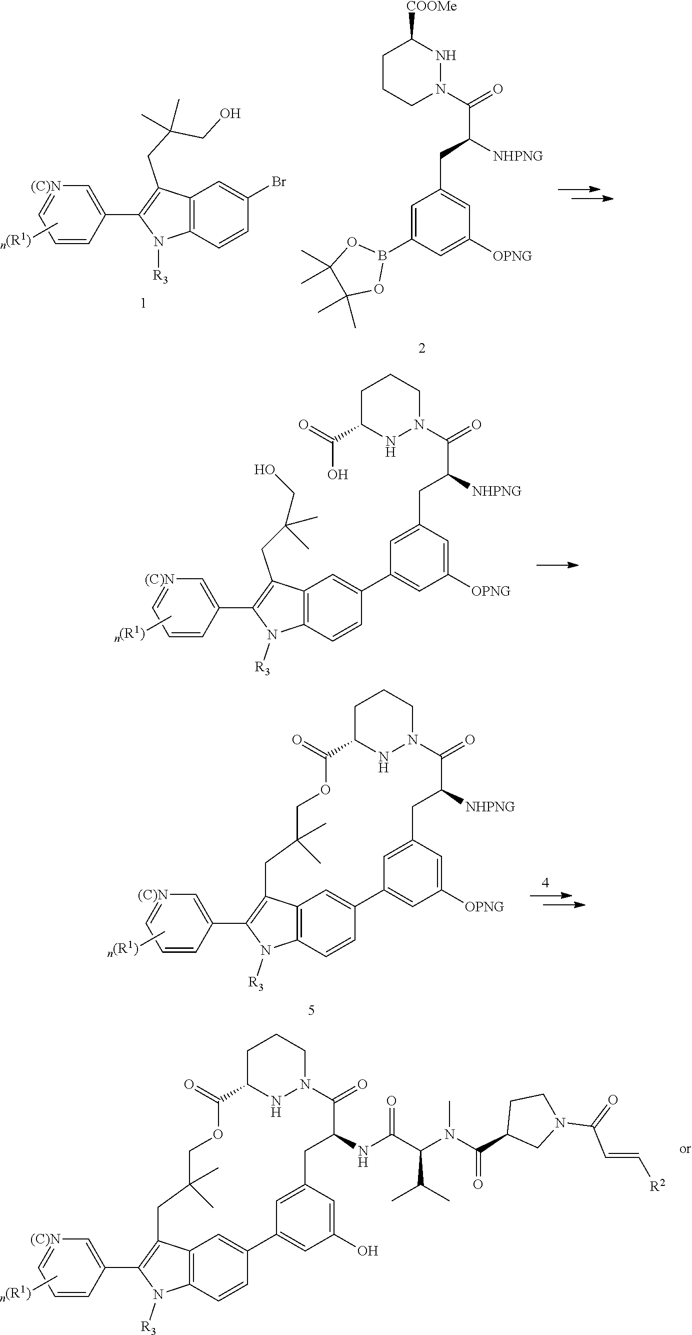

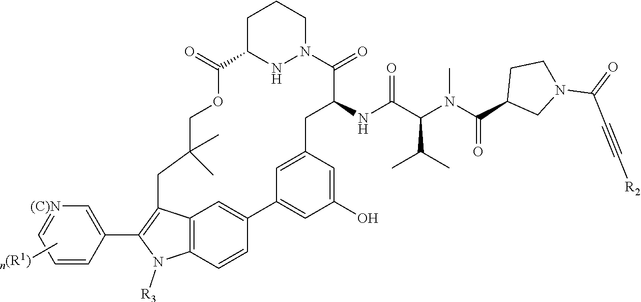

A general synthesis of macrocyclic esters is outlined in Scheme B1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl methyl (S)-hexahydropyridazine-3-carboxylate.

An appropriately substituted acetylpyrrolidine-3-carbonyl-N-methyl-L-valine (or an alternative aminoacid derivative (4) can be made by coupling of methyl-L-valinate and protected (S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection and/or functionalization steps can be required to produce the final compound.

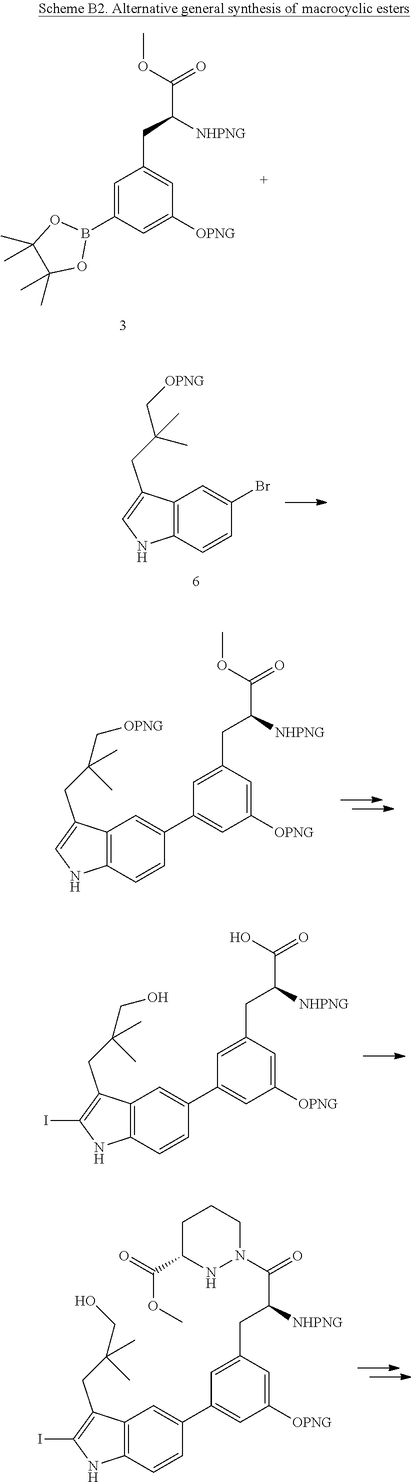

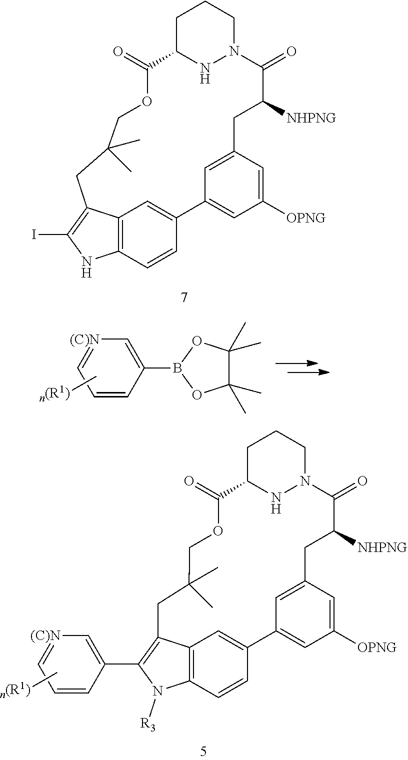

Alternatively, macrocyclic ester can be prepared as described in Scheme B2. An appropriately protected bromo-indolyl (6) coupled in the presence of a Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of a Pd catalyst with an appropriately substituted boronic ester and alkyllation can yield fully protected macrocycle (5). Additional deprotection or functionalization steps are required to produce the final compound.

In addition, compounds of the disclosure can be synthesized using the methods described in the Examples below or as described in WO 2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and W are defined herein, including by using methods exemplified in the Example section herein and in WO 2021/091982.

Compounds of Table B1 herein were prepared using methods disclosed herein or were prepared using methods disclosed herein combined with the knowledge of one of skill in the art. Compounds of Table B2 may be prepared using methods disclosed herein or may be prepared using methods disclosed herein combined with the knowledge of one of skill in the art.

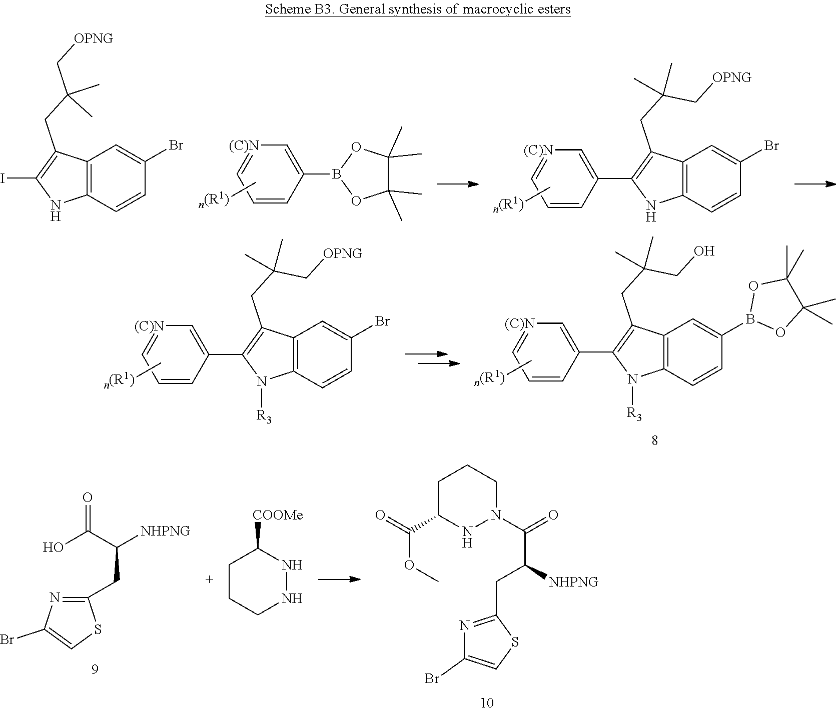

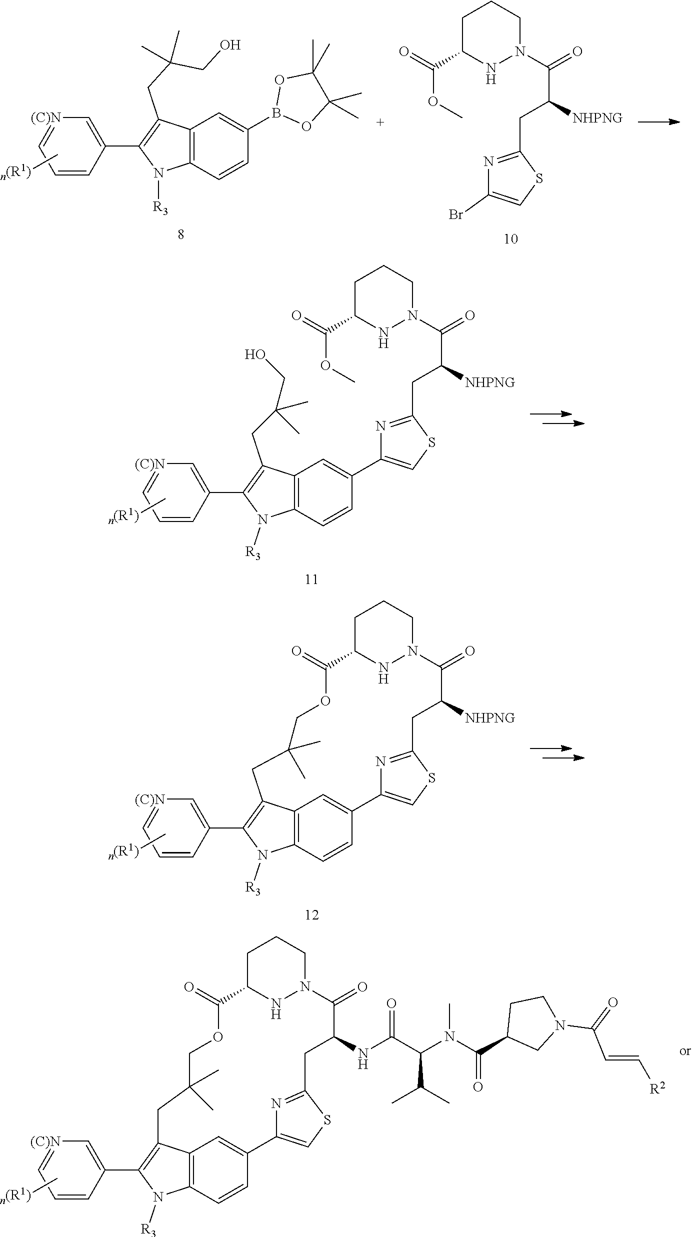

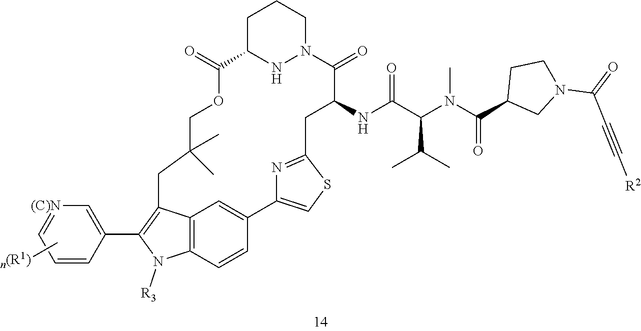

An alternative general synthesis of macrocyclic esters is outlined in Scheme B3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S′)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

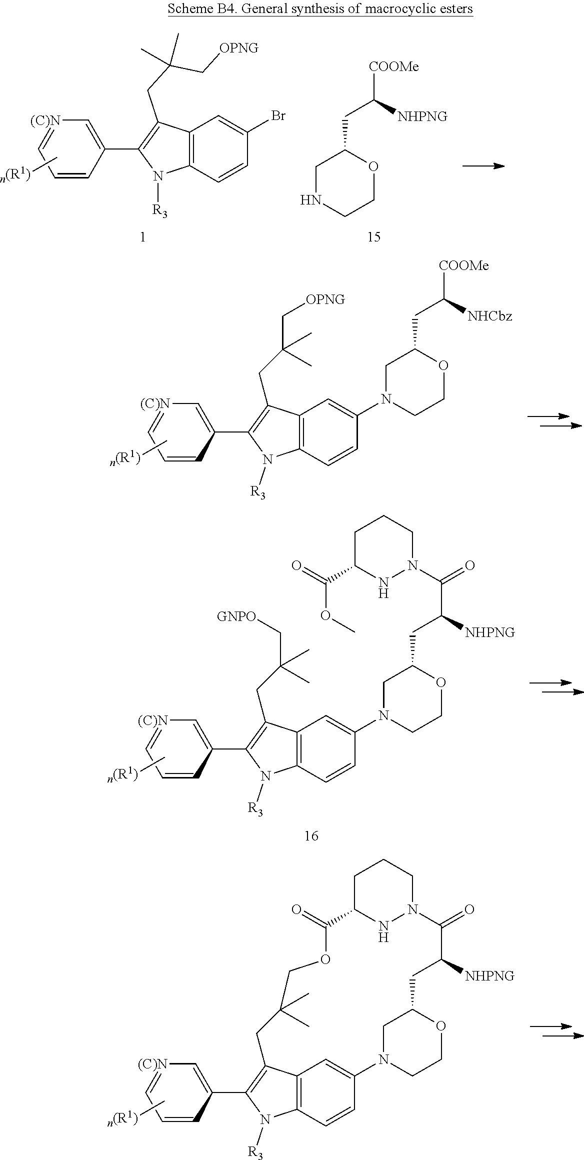

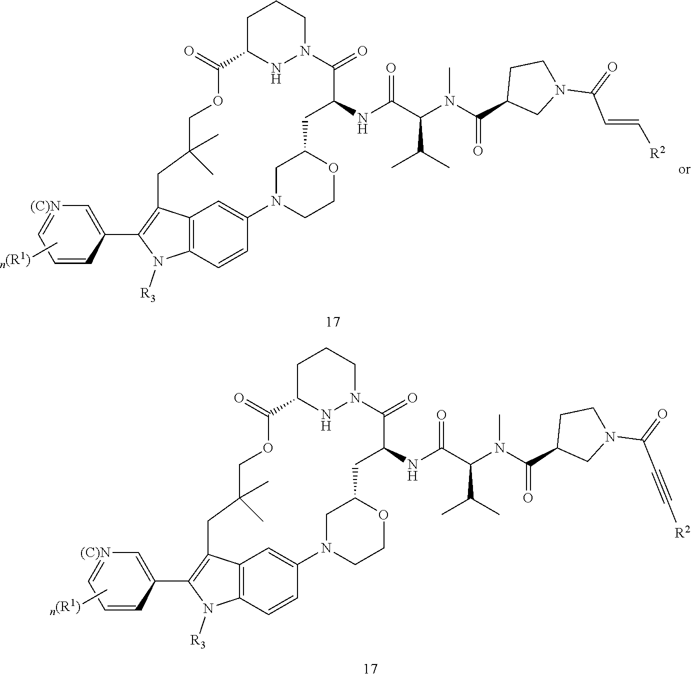

An alternative general synthesis of macrocyclic esters is outlined in Scheme B4. An appropriately substituted morpholine or an alternative herecyclic intermediate (15) can be coupled with appropriately protected Intermediate 1 via Palladium mediated coupling. Subsequent ester hydrolysis, and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

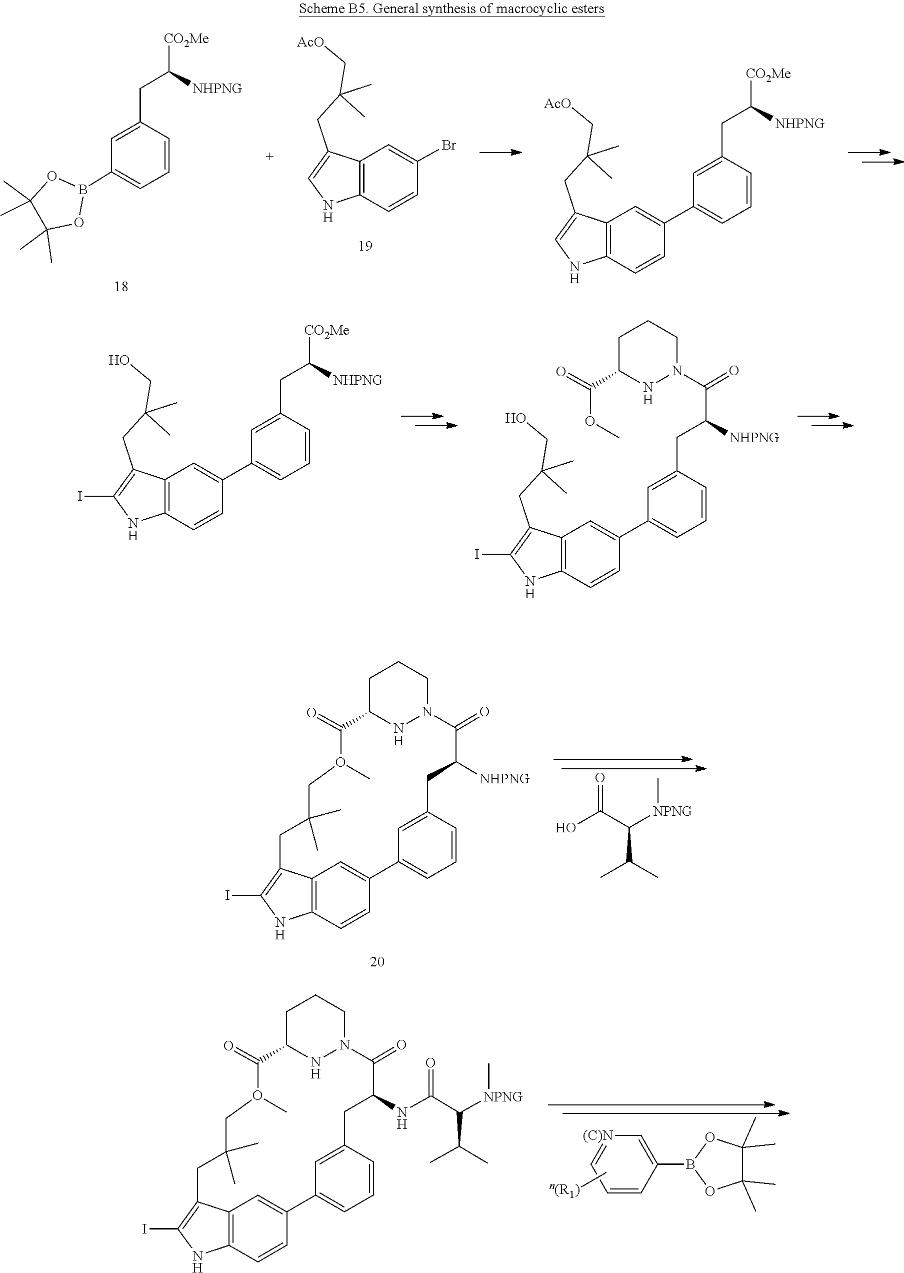

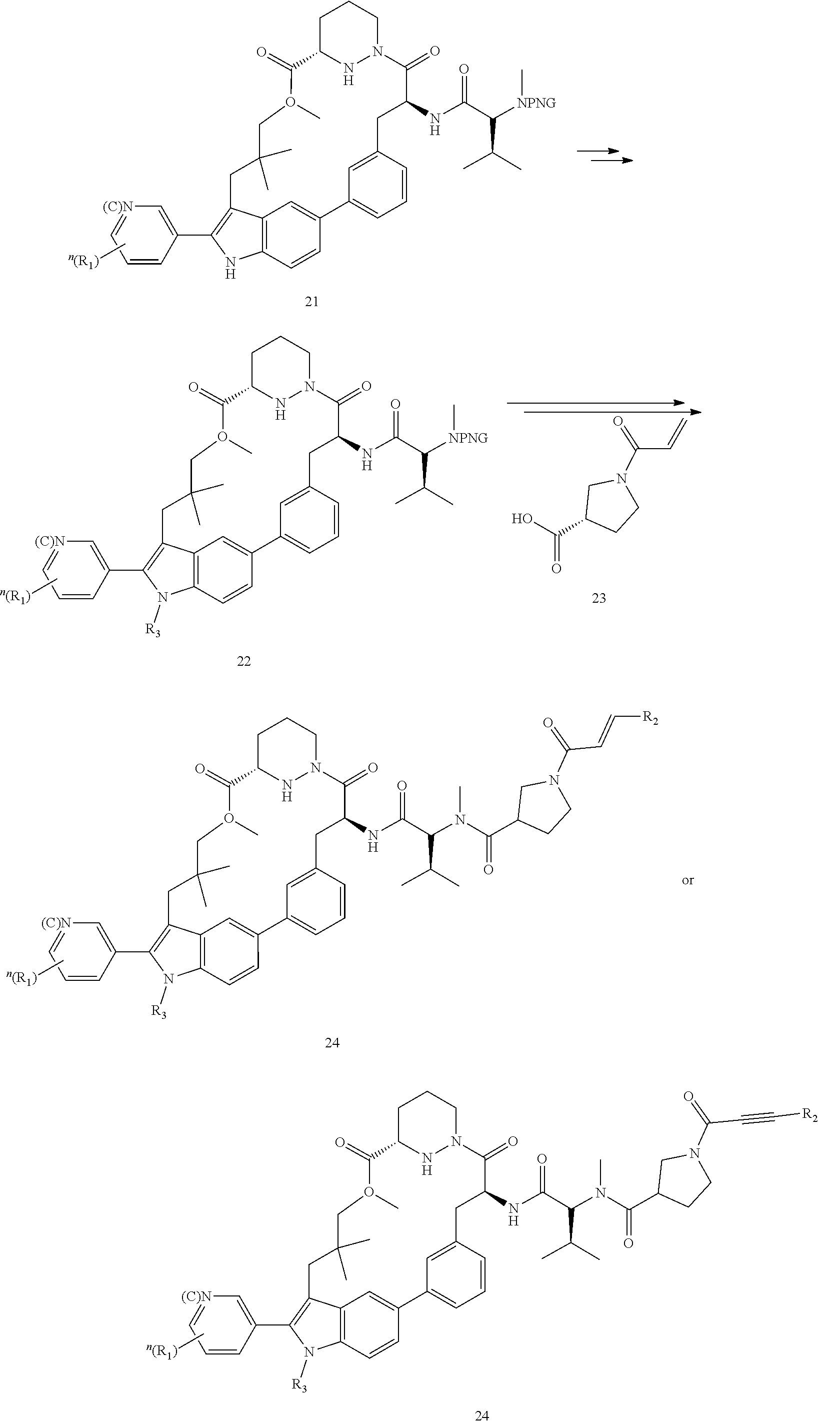

An alternative general synthesis of macrocyclic esters is outlined in Scheme B5. An appropriately substituted macrocycle (20) can be prepared starting from an appropriately protected boronic ester 18 and bromo indolyl intermediate (19), including Palladium mediated coupling, hydrolysis, coupling with piperazoic ester, hydrolysis, de-protection, and macrocyclizarion steps. Subsequent coupling with an appropriately substituted protected aminoacid followed by palladium mediated coupling yields intermediate 21. Additional deprotection and derivatization steps, including 6611kylation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below and in WO2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and W are defined herein, including by using methods exemplified in the WO 2021/091982.

In some embodiments, the RAS inhibitor is a compound disclosed in WO 2021/091967.

In some embodiments, the RAS(ON) inhibitor is a compound, or a pharmaceutically acceptable salt thereof, having the structure of Formula CI.

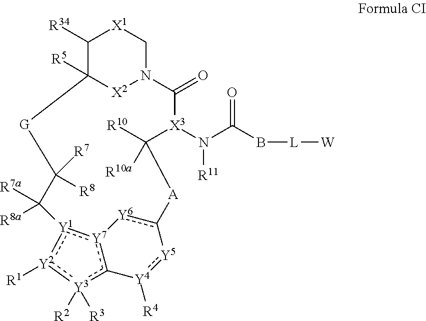

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an A-ethoxycarboiyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)2R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl (e.g., methyl).

In some embodiments of Formula CI and subformula thereof, R⁹ is optionally substituted C1-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, R³⁴ is hydrogen.

In some embodiments of Formula CI and subformula thereof, G is optionally substituted C₁-C₄ heteroalkylene.

In some embodiments, the RAS(ON) inhibitor has the structure of Formula CIa, or a pharmaceutically acceptable salt thereof:

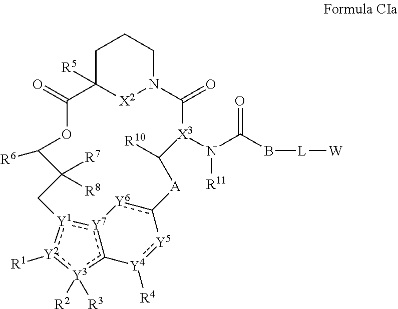

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula CI and subformula thereof, X² is NH. In some embodiments, X³ is CH.

In some embodiments of Formula CI and subformula thereof, R¹¹ is hydrogen. In some embodiments, R¹¹ is C₁-C₃ alkyl, such as methyl.

In some embodiments, the RAS(ON) inhibitor has the structure of Formula CIb, or a pharmaceutically acceptable salt thereof:

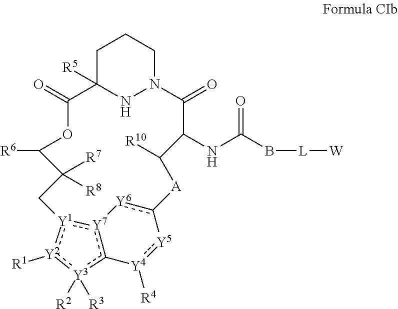

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula CI and subformula thereof, X¹ is optionally substituted C₁-C₂ alkylene. In some embodiments, X¹ is methylene.

In some embodiments of Formula CI and subformula thereof, R⁴ is hydrogen.

In some embodiments of Formula CI and subformula thereof, R⁵ is hydrogen. In some embodiments, R⁵ is C₁-C₄ alkyl optionally substituted with halogen. In some embodiments, R⁵ is methyl.

In some embodiments of Formula CI and subformula thereof, Y⁴ is C.

In some embodiments of Formula CI and subformula thereof, R⁴ is hydrogen.

In some embodiments of Formula CI and subformula thereof, Y⁵ is CH.

In some embodiments of Formula CI and subformula thereof, Y⁶ is CH.

In some embodiments of Formula CI and subformula thereof, Y¹ is C.

In some embodiments of Formula CI and subformula thereof, Y² is C.

In some embodiments of Formula CI and subformula thereof, Y³ is N.

In some embodiments of Formula CI and subformula thereof, R³ is absent.

In some embodiments of Formula CI and subformula thereof, Y⁷ is C.

In some embodiments, the RAS(ON) inhibitor has the structure of Formula CIc, or a pharmaceutically acceptable salt thereof:

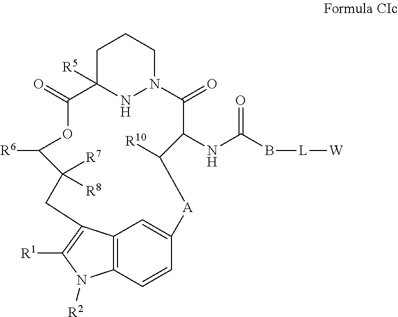

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ, an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula CI and subformula thereof, R⁶ is hydrogen.

In some embodiments of Formula CI and subformula thereof, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl, such as ethyl.

In some embodiments of Formula CI and subformula thereof, R⁷ is optionally substituted C₁-C₃ alkyl. In some embodiments, R⁷ is C₁-C₃ alkyl.

In some embodiments of Formula CI and subformula thereof, R⁸ is optionally substituted C₁-C₃ alkyl. In some embodiments, R⁸ is C₁-C₃ alkyl.

In some embodiments, the RAS(ON) inhibitor has the structure of Formula CId, or a pharmaceutically acceptable salt thereof:

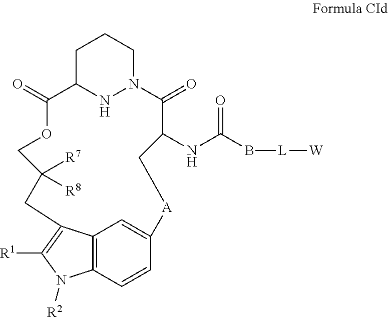

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, R⁸ is 5 to 10-membered heteroaryl. In some embodiments, R′ is optionally substituted 6-membered aryl or optionally substituted 6-membered heteroaryl.

In some embodiments, the RAS(ON) inhibitor has the structure of Formula CIe, or a pharmaceutically acceptable salt thereof:

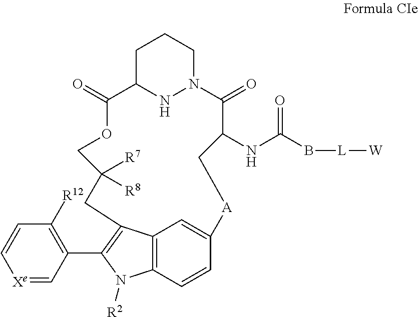

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)—where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} is Nor CH; and
R¹² is optionally substituted C₁-C₆ alkyl or optionally substituted C₁-C₆ heteroalkyl.

In some embodiments of Formula CI and subformula thereof, X^{e} is N. In some embodiments, X^{e} is CH.

In some embodiments of Formula CI and subformula thereof, R¹² is optionally substituted C₁-C₆ heteroalkyl. In some embodiments, R¹² is

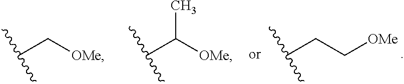

In some embodiments, R¹² is

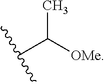 [SMILES: *C(C)OC]

In some embodiments, the RAS(ON) inhibitor has the structure of Formula CIf, or a pharmaceutically acceptable salt thereof:

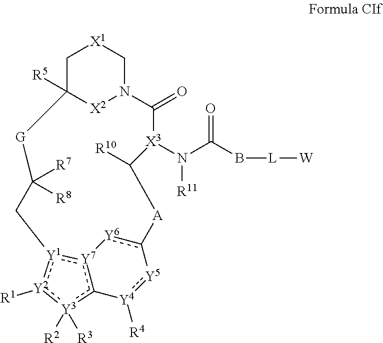

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the RAS(ON) inhibitor has the structure of Formula CVI, or a pharmaceutically acceptable salt thereof:

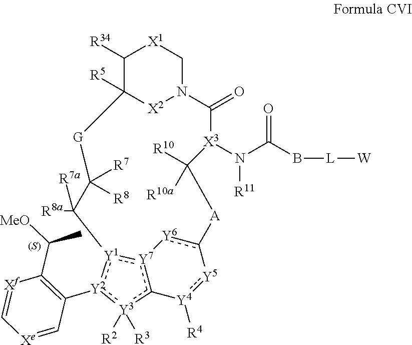

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an A-ethoxycarboiyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R³⁴ is hydrogen or C₁-C₃ alkyl; and
X^{e} and X^{f} are, independently, N or CH.

In some embodiments, the RAS(ON) inhibitor has the structure of Formula CVIa, or a pharmaceutically acceptable salt thereof:

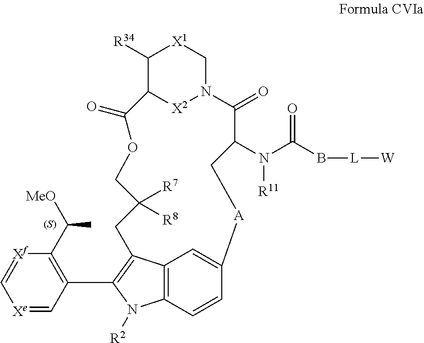

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of Formula CI and subformula thereof, X^{e} is N and X^{f} is CH. In some embodiments, X^{e} is CH and X^{f} is N.

In some embodiments, the RAS(ON) inhibitor has the structure of Formula CVIb, or a pharmaceutically acceptable salt thereof:

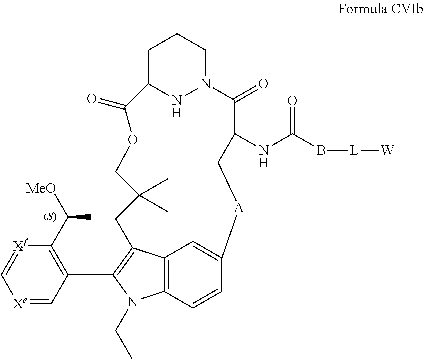

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroqluinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.

In some embodiments of Formula CI and subformula thereof, X is N and X^{f} is CH. In some embodiments, X is CH and X^{f} is N.

In some embodiments, the RAS(ON) inhibitor has the structure of Formula CVII, or a pharmaceutically acceptable salt thereof:

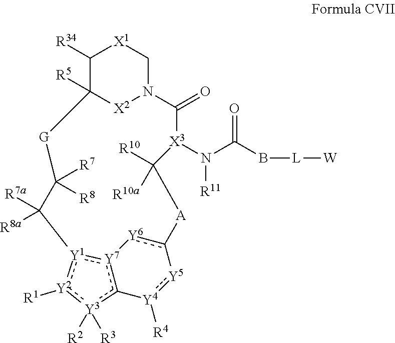

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R)—, —C(O)NH—CH(R⁶)— where C is bound to —C(R⁷R)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is

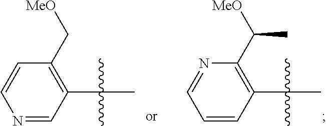 [SMILES: *c1cnccc1COC ; *c1cccnc1[C@H](C)OC]

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl (e.g., methyl).

In some embodiments of Formula CI and subformula thereof, A is optionally substituted 6-membered arylene. In some embodiments, A has the structure:

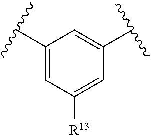

\wherein R¹³ is hydrogen, hydroxy, amino, optionally substituted C₁-C₆ alkyl, or optionally substituted C1-C₆ heteroalkyl. In some embodiments, R¹³ is hydrogen. In some embodiments, R¹³ is hydroxy.

In some embodiments of Formula CI and subformula thereof, B is —CHR⁹—. In some embodiments, R⁹ is optionally substituted C₁-C₆ alkyl or optionally substituted 3 to 6-membered cycloalkyl.

In some embodiments, R⁹ is

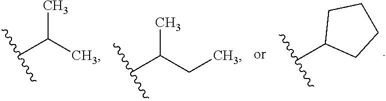

In some embodiments, R⁹ is

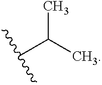 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, B is optionally substituted 6-membered arylene.

In some embodiments, B is 6-membered arylene.

In some embodiments, B is:

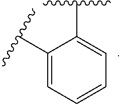 [SMILES: *c1ccccc1*]

In some embodiments of Formula CI and subformula thereof, R⁷ is methyl.

In some embodiments of Formula CI and subformula thereof, R⁸ is methyl.

In some embodiments of Formula CI and subformula thereof, R³⁴ is hydrogen.

In some embodiments of Formula CI and subformula thereof, the linker is the structure of Formula CII:

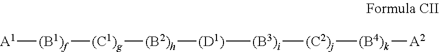

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹—(B¹)^{f}—(C¹)_{g}—(B²)ₕ— to —(B³)ᵢ—(C²)ⱼ—(B⁴)ₖ-A².

In some embodiments of Formula CI and subformula thereof, the linker is acyclic.

In some embodiments of Formula CI and subformula thereof, the linker has the structure of Formula CIIa:

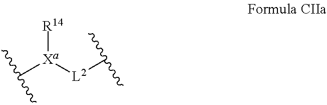

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

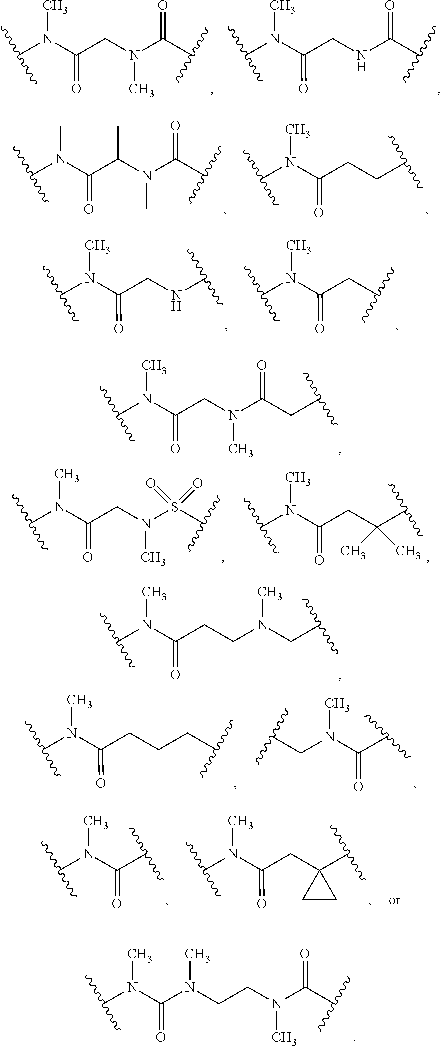

In some embodiments of Formula CI and subformula thereof, the linker is or a comprises a cyclic group.

In some embodiments, the linker has the structure of Formula CIIb:

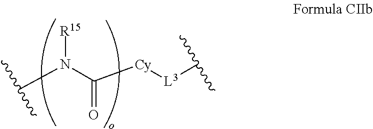

wherein o is 0 or 1;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments, the linker has the structure:

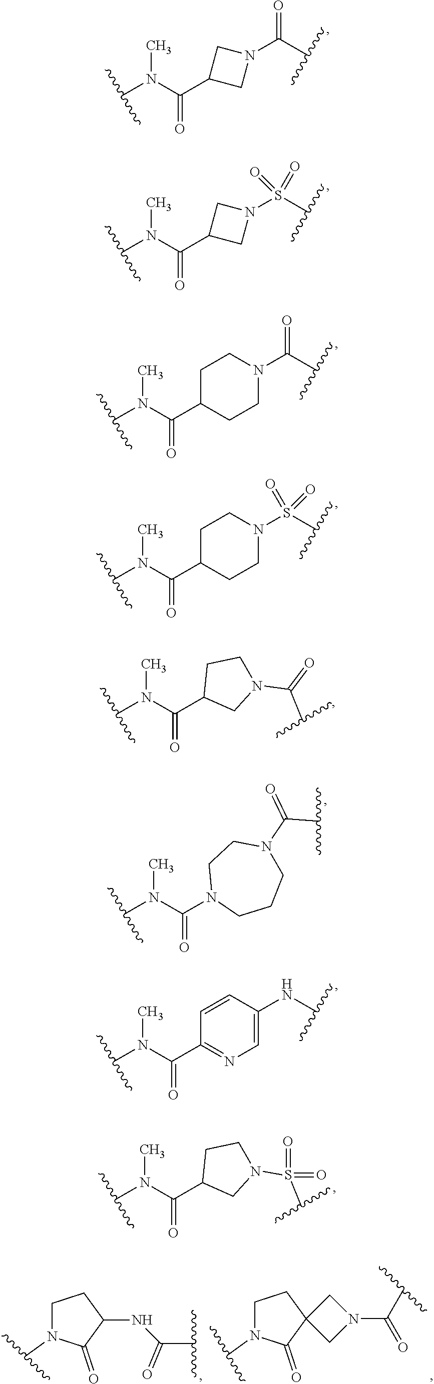

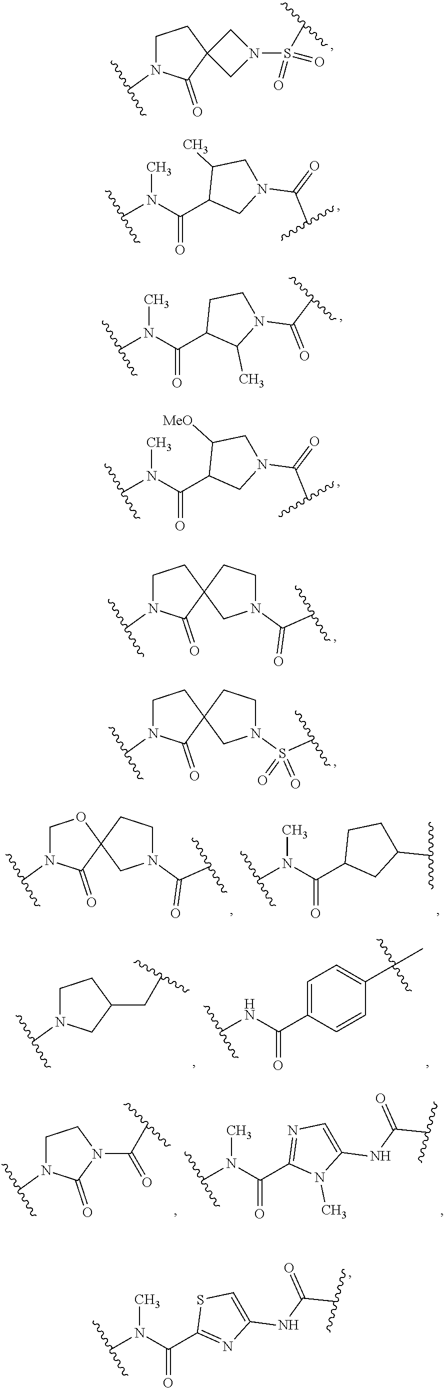

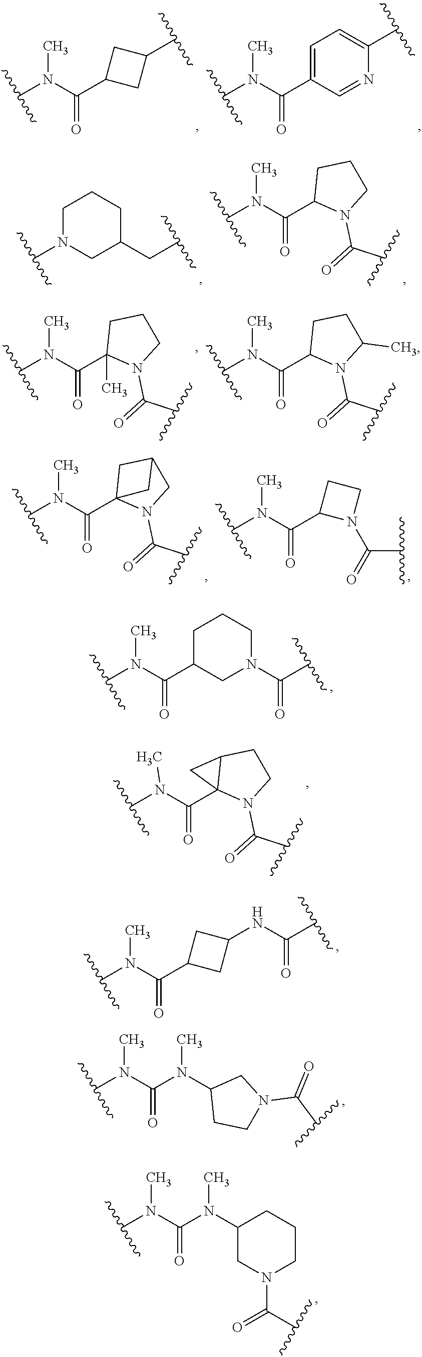

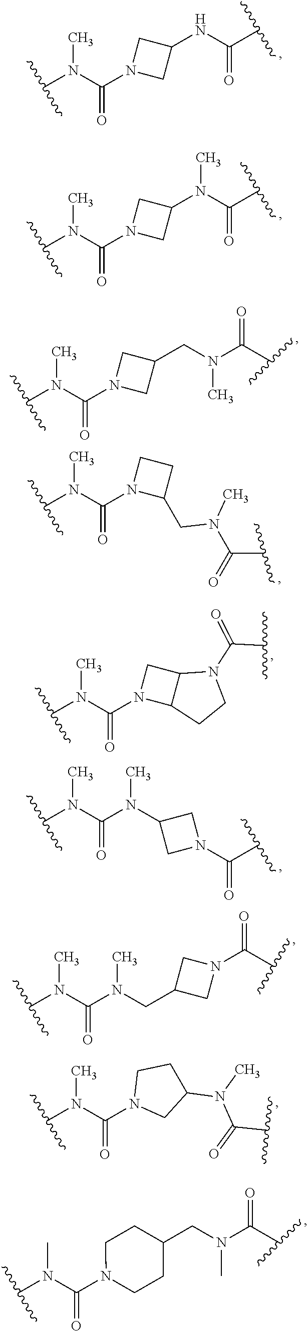

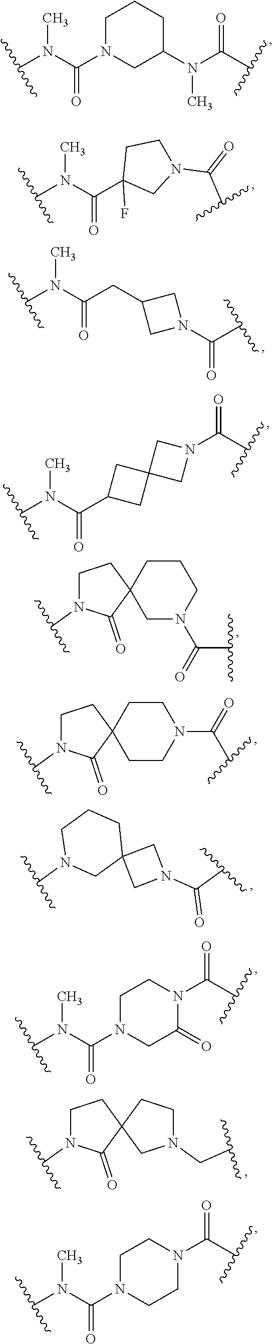

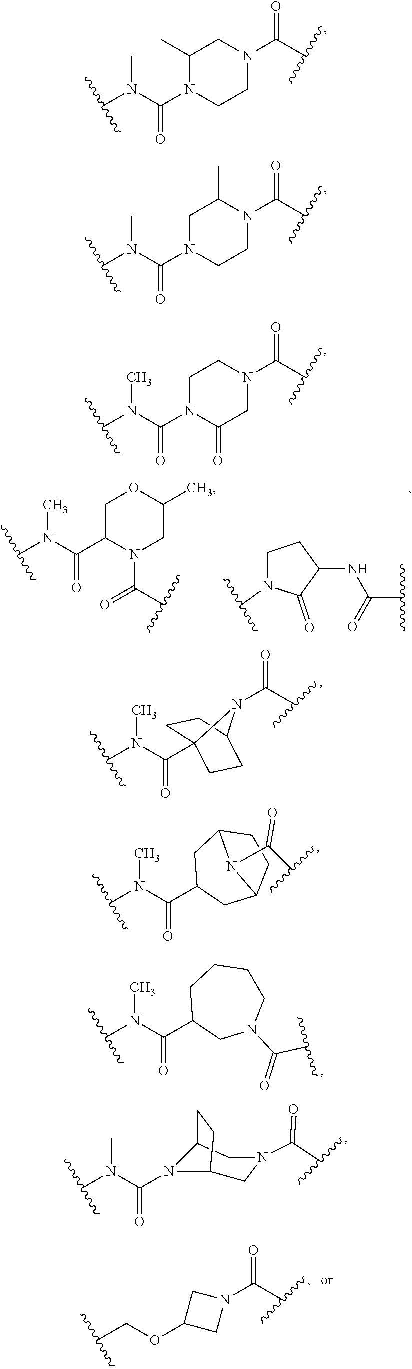

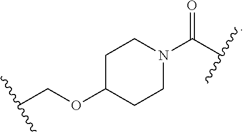 [SMILES: *COC1CCN(C(*)=O)CC1]

In some embodiments, a linker of Formula CII is selected from the group consisting of

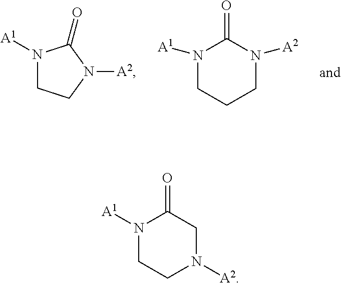

In some embodiments of Formula CI and subformula thereof, R⁴ is hydrogen.

In some embodiments of Formula CI and subformula thereof, W comprises a carbodiimide.

In some embodiments, W has the structure of Formula CIIIa:

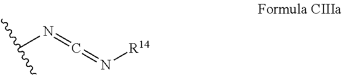

wherein R¹⁴ is optionally substituted C₁-C₆ alkyl, optionally substituted C₁-C₆ heteroalkyl, optionally substituted 3 to 8-membered cycloalkyl, optionally substituted 6 to 10-membered aryl, optionally substituted 3 to 14-membered heterocycloalkyl, or optionally substituted 5 to 10-membered heteroaryl.

In some embodiments, W has the structure:

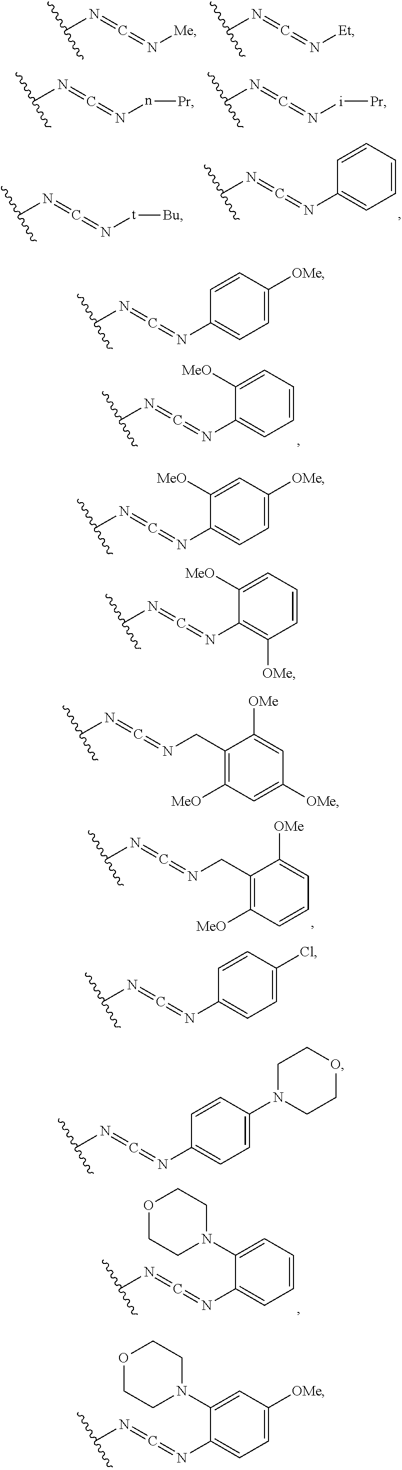

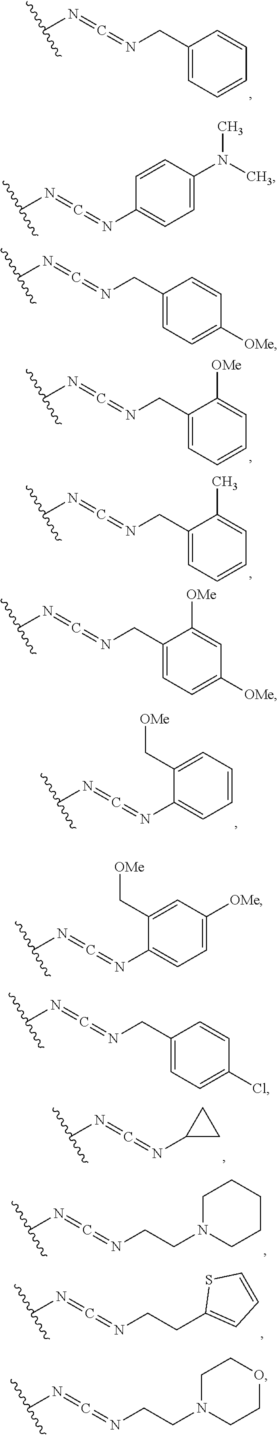

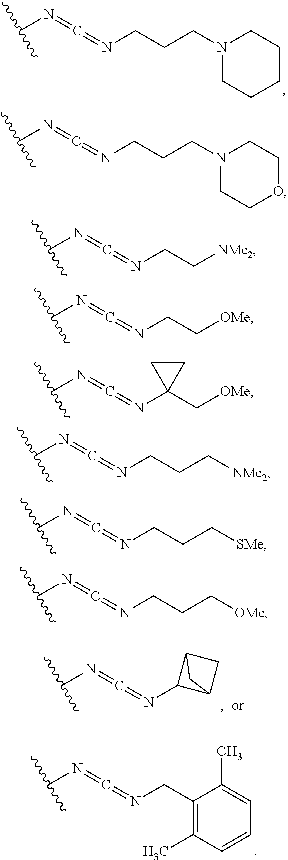

In some embodiments of Formula CI and subformula thereof, W comprises an oxazoline or thiazoline. In some embodiments, W has the structure of Formula CIIIb:

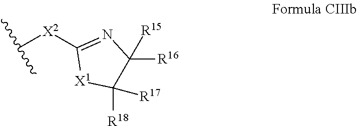

wherein X¹ is O or S;
X² is absent or NR¹⁹
R¹⁵, R¹⁶, R¹⁷, and R¹⁸ are, independently, hydrogen or optionally substituted C₁-C₆ alkyl; and
R¹⁹ is hydrogen, C(O)(optionally substituted C₁-C₆ alkyl), optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

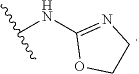 [SMILES: *NC1=NCCO1]

In some embodiments of Formula CI and subformula thereof, W comprises a chloroethyl urea, a chloroethyl thiourea, a chloroethyl carbamate, or a chloroethyl thiocarbamate. In some embodiments, W has the structure of Formula CIIIc:

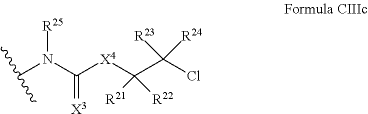

wherein X³ is O or S;
X⁴ is O, S, NR²⁶;
R²¹, R²², R²³, R²⁴, and R²⁶ are, independently, hydrogen or optionally substituted C₁-C₆ alkyl; and
R²⁵ is hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

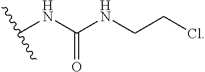 [SMILES: *NC(=O)NCCCl]

In some embodiments of Formula CI and subformula thereof, W comprises an aziridine. In some embodiments, W has the structure of Formula CIIId1, Formula CIIId2, Formula CIIId3, or Formula CIIId4:

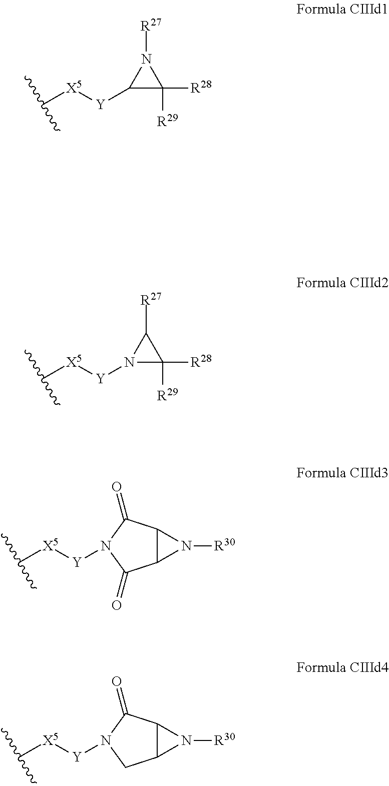

wherein X⁵ is absent or NR³⁰;
Y is absent or C(O), C(S), S(O), SO₂, or optionally substituted C₁-C₃ alkylene;
R²⁷ is hydrogen, —C(O)R³², —C(O)OR³², —SOR³³, —SO₂R³³, optionally substituted C₁-C₆ alkyl, optionally substituted 6 to 10-membered aryl, optionally substituted 3 to 14-membered heterocycloalkyl, or optionally substituted 5 to 10-membered heteroaryl; R²⁸ and R²⁹ are, independently, hydrogen, CN, C(O)R³¹, CO₂R³¹, C(O)R³¹R³¹ optionally substituted C₁-C₆ alkyl, optionally substituted 3 to 10-membered cycloalkyl, optionally substituted 6 to 10-membered aryl, optionally substituted 3 to 14-membered heterocycloalkyl, or optionally substituted 5 to 10-membered heteroaryl;
each R³¹ is, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl;
R³⁰ is hydrogen or optionally substituted C₁-C₆ alkyl; and
R³² and R³³ are, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is:

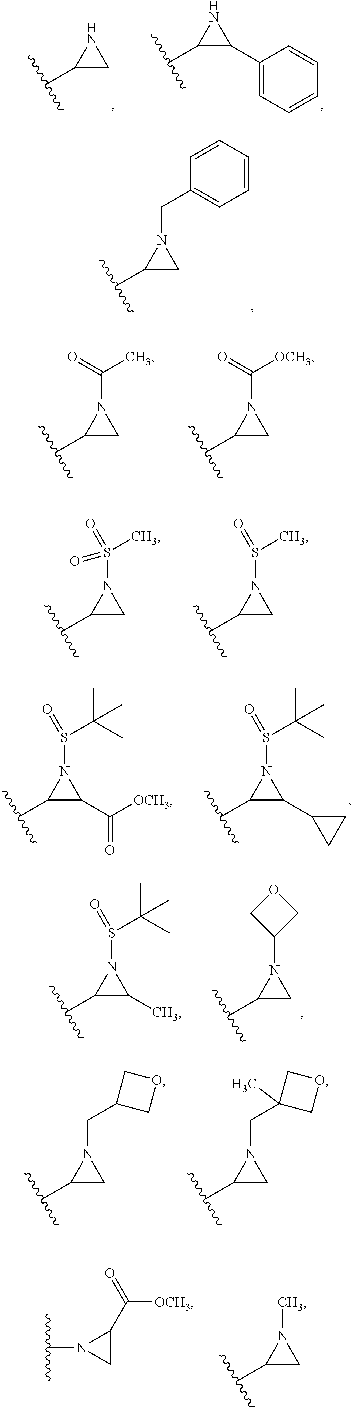

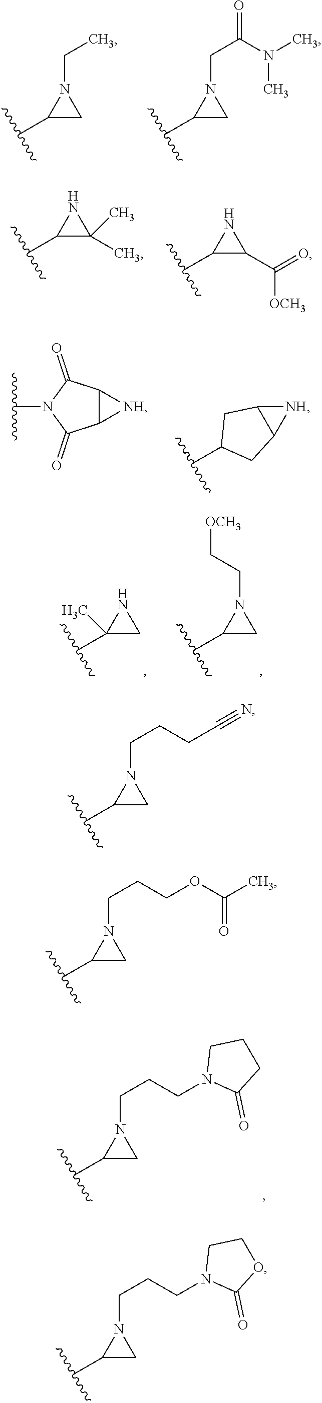

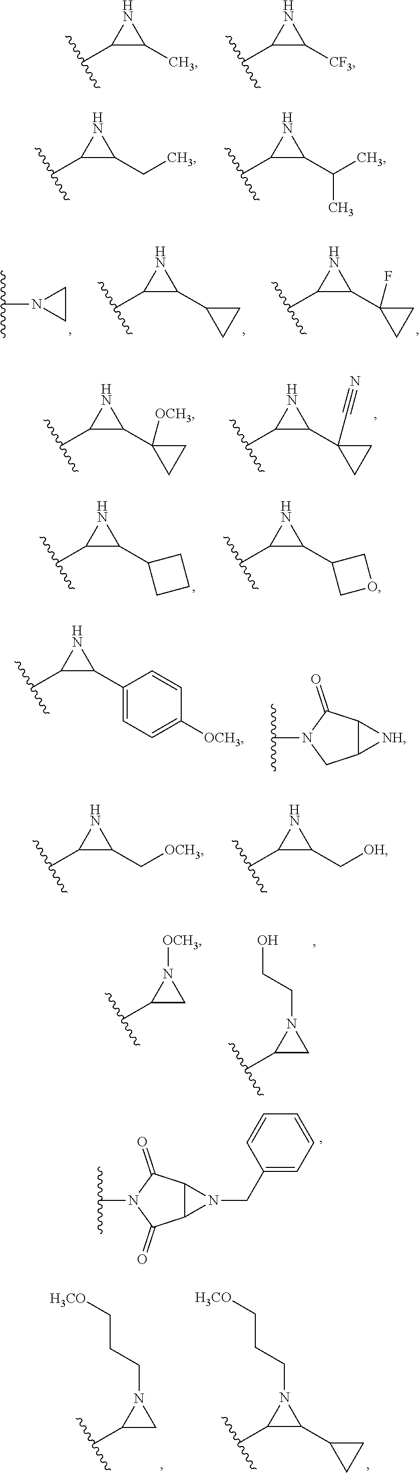

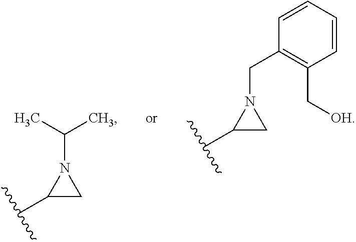

In some embodiments of Formula CI and subformula thereof, W comprises an epoxide. In some embodiments, W is

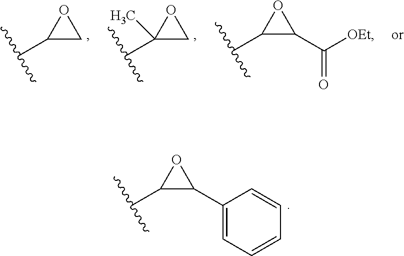

In some embodiments of Formula CI and subformula thereof, W is a cross-linking group bound to an organic moiety that is a Ras binding moiety, i.e., RBM-W, wherein upon contact of an RBM-W compound with a Ras protein, the RBM-W binds to the Ras protein to form a conjugate. For example, the W moiety of an RBM-W compound may bind, e.g., cross-link, with an amino acid of the Ras protein to form the conjugate. In some embodiments, the Ras binding moiety is a K-Ras binding moiety. In some embodiments, the K-Ras binding moiety binds to a residue of a K-Ras Switch-II binding pocket of the K-Ras protein. In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein. The W of an RBM-W compound may comprise any W described herein. The Ras binding moiety typically has a molecular weight of under 1200 Da. See, e.g., see, e.g., Johnson et al., 292:12981-12993 (2017) for a description of Ras protein domains, incorporated herein by reference.

In some embodiments, the RAS(ON) inhibitor is selected from Table C1, or a pharmaceutically acceptable salt or stereoisomer thereof. In some embodiments, the RAS(ON) inhibitor is selected from Table C1, or a pharmaceutically acceptable salt or atropisomer thereof.

TABLE C1
Certain Compounds of the Present Invention

| Ex # | Structure |
|---|---|
| C1 | 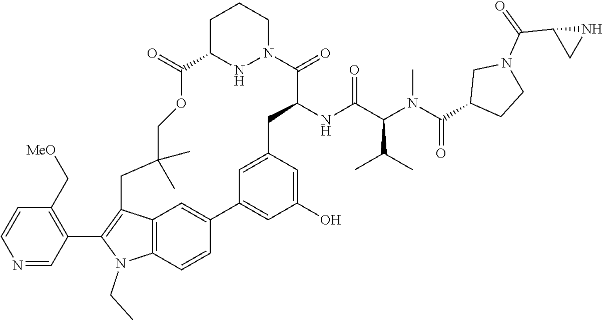 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@H]3CN3)C1)C(=O)N1CCC[C@H](N1)C(=O)OCC(C)(C)C2] |
| C2 | 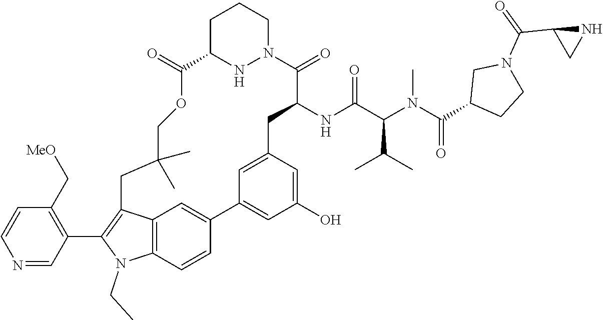 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CN3)C1)C(=O)N1CCC[C@H](N1)C(=O)OCC(C)(C)C2] |
| C3 | 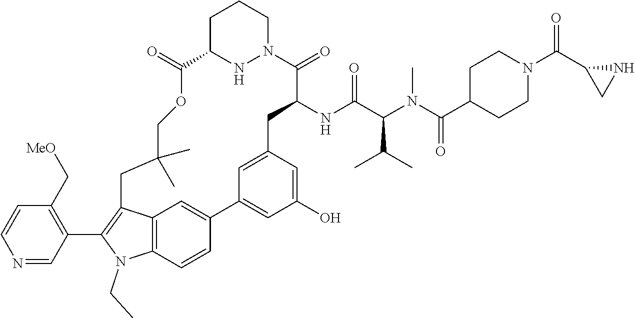 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@H]3CN3)CC1)C(=O)N1CCC[C@H](N1)C(=O)OCC(C)(C)C2] |
| C4 | 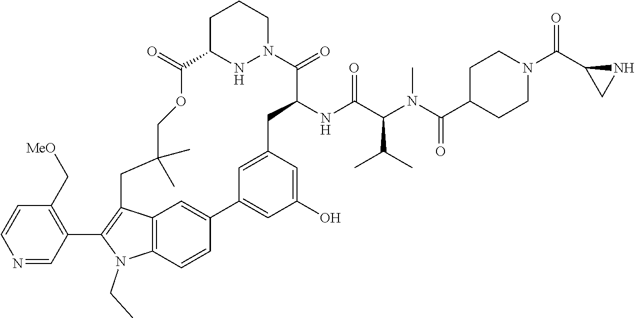 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@@H]3CN3)CC1)C(=O)N1CCC[C@H](N1)C(=O)OCC(C)(C)C2] |
| C5 | 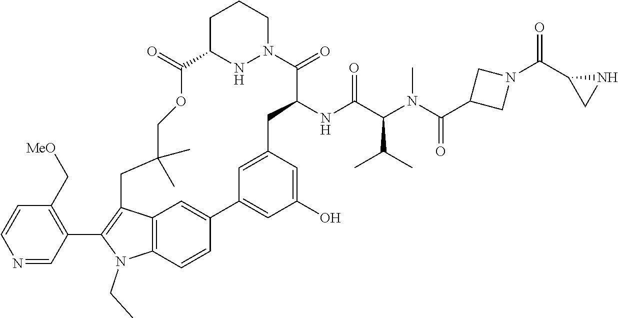 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@H]3CN3)C1)C(=O)N1CCC[C@H](N1)C(=O)OCC(C)(C)C2] |
| C6 | 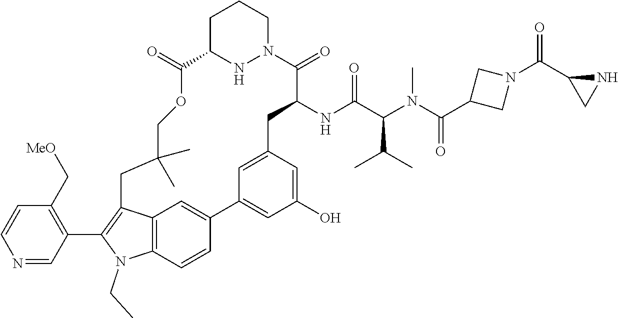 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@@H]3CN3)C1)C(=O)N1CCC[C@H](N1)C(=O)OCC(C)(C)C2] |
| C7 | 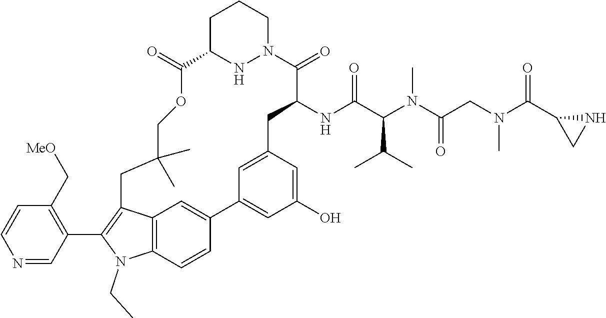 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1)C(=O)N1CCC[C@H](N1)C(=O)OCC(C)(C)C2] |
| C8 | 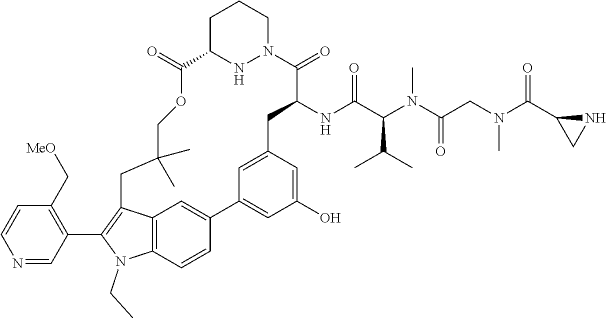 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1)C(=O)N1CCC[C@H](N1)C(=O)OCC(C)(C)C2] |
| C9 | 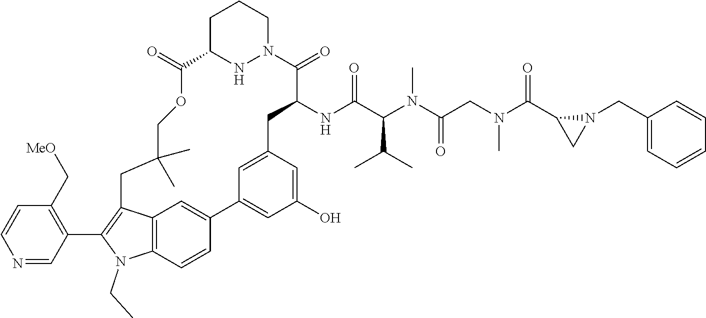 |
| C10 | 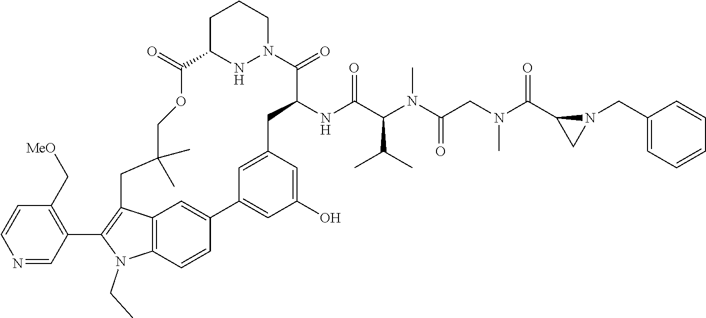 |
| C11 | 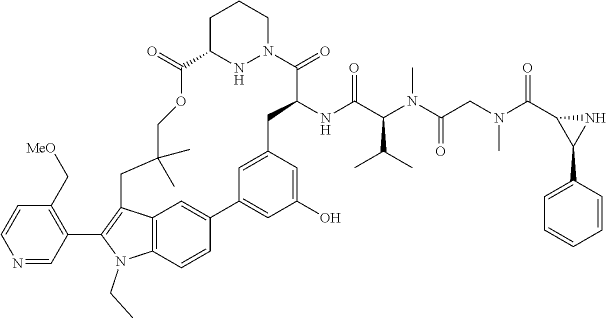 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccccc1)C(=O)N1CCC[C@H](N1)C(=O)OCC(C)(C)C2] |
| C12 | 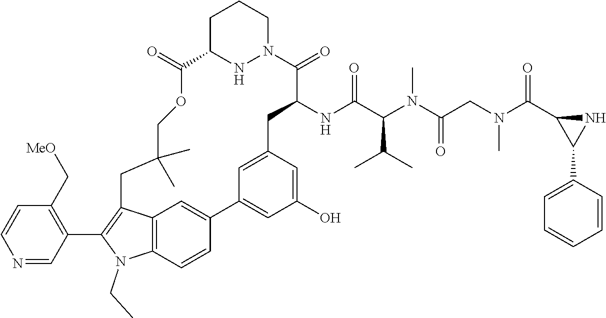 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccccc1)C(=O)N1CCC[C@H](N1)C(=O)OCC(C)(C)C2] |
| C13 | 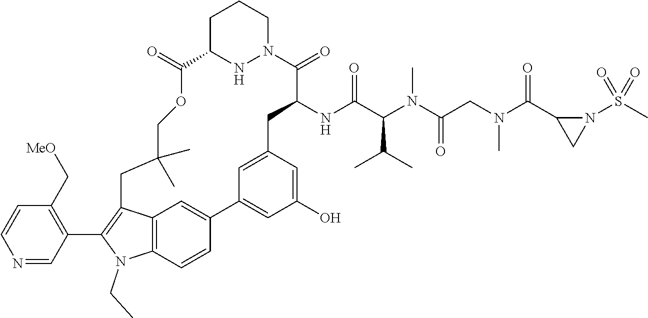 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1S(C)(=O)=O)C(=O)N1CCC[C@H](N1)C(=O)OCC(C)(C)C2] |
| C14 | 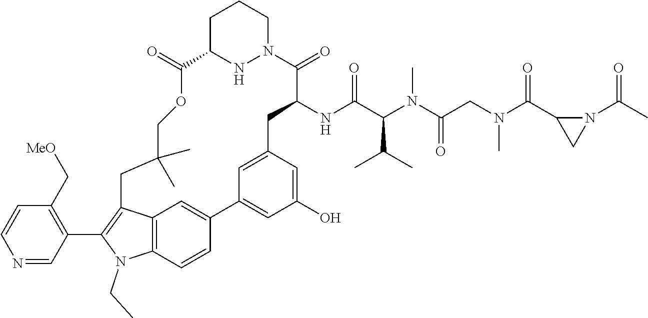 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1C(C)=O)C(=O)N1CCC[C@H](N1)C(=O)OCC(C)(C)C2] |
| C15 A and B | 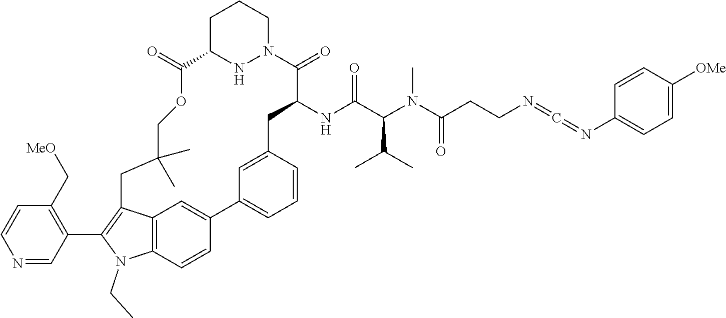 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1)C(=O)N1CCC[C@H](N1)C(=O)OCC(C)(C)C2] |
| C16 A and B | 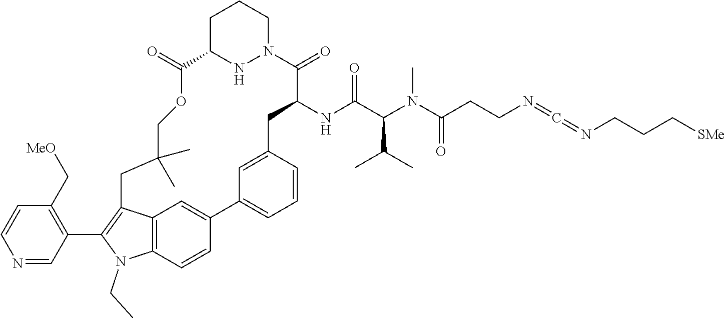 [SMILES: CCn1c(-c2cnccc2COC)c2c3cc(ccc31)-c1cccc(c1)C[C@H](NC(=O)[C@H](C(C)C)N(C)C(=O)CCN=C=NCCCSC)C(=O)N1CCC[C@H](N1)C(=O)OCC(C)(C)C2] |
| C17 A and B | 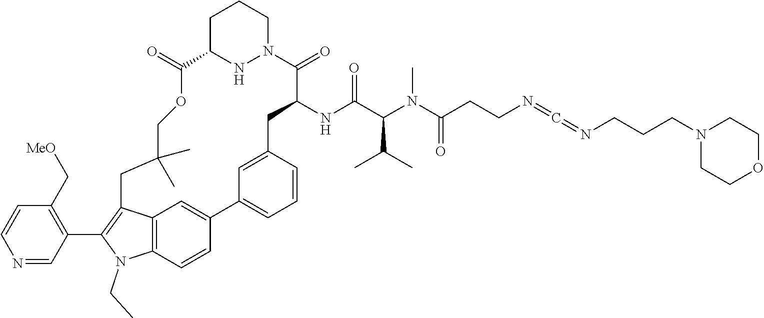 [SMILES: CCn1c(-c2cnccc2COC)c2c3cc(ccc31)-c1cccc(c1)C[C@H](NC(=O)[C@H](C(C)C)N(C)C(=O)CCN=C=NCCCN1CCOCC1)C(=O)N1CCC[C@H](N1)C(=O)OCC(C)(C)C2] |
| C18 A and B | 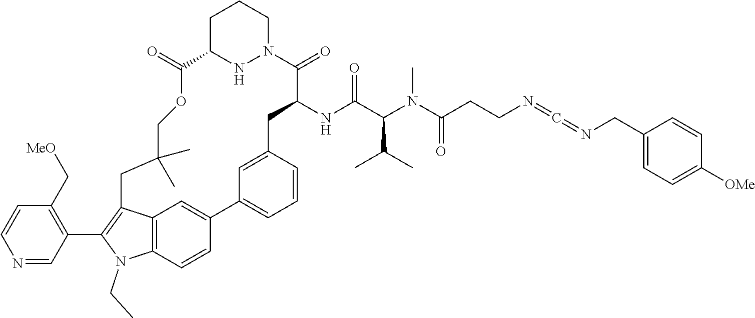 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1)C(=O)N1CCC[C@H](N1)C(=O)OCC(C)(C)C2] |
| C19 A and B | 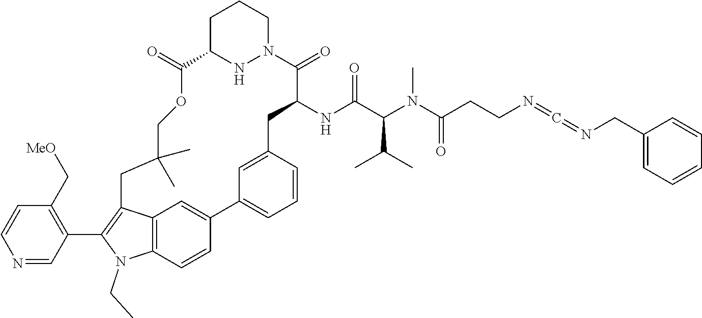 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1)C(=O)N1CCC[C@H](N1)C(=O)OCC(C)(C)C2] |
| C20 A and B | 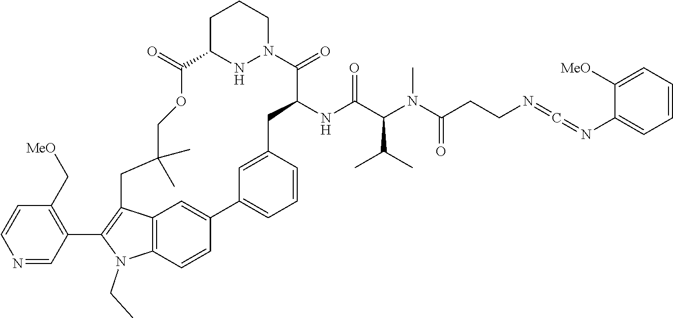 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1OC)C(=O)N1CCC[C@H](N1)C(=O)OCC(C)(C)C2] |
| C21 A and B | 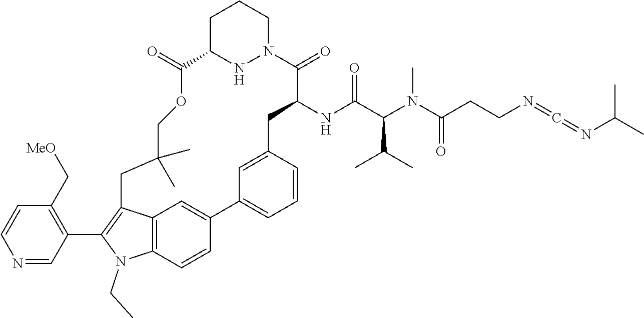 [SMILES: CCn1c(-c2cnccc2COC)c2c3cc(ccc31)-c1cccc(c1)C[C@H](NC(=O)[C@H](C(C)C)N(C)C(=O)CCN=C=NC(C)C)C(=O)N1CCC[C@H](N1)C(=O)OCC(C)(C)C2] |
| C22 A and B | 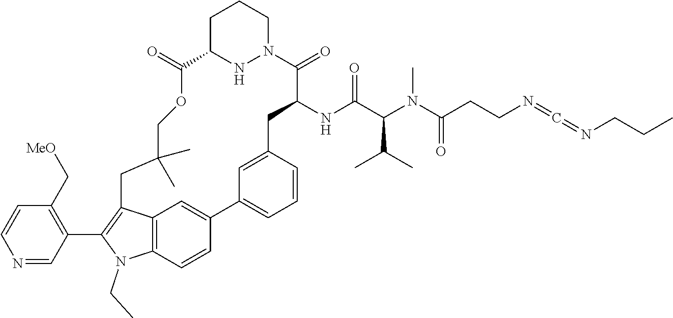 [SMILES: CCCN=C=NCCC(=O)N(C)[C@H](C(=O)N[C@H]1Cc2cccc(c2)-c2ccc3c(c2)c(c(-c2cnccc2COC)n3CC)CC(C)(C)COC(=O)[C@@H]2CCCN(N2)C1=O)C(C)C] |
| C23 A and B | 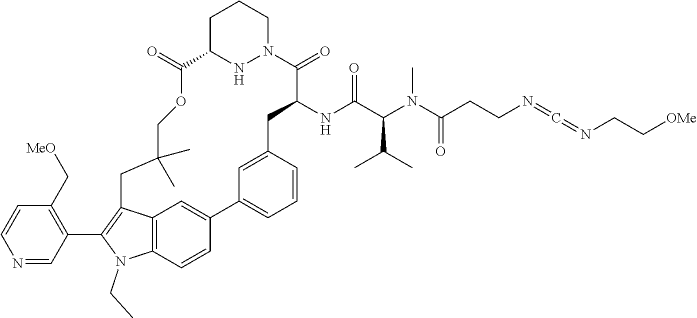 [SMILES: CCn1c(-c2cnccc2COC)c2c3cc(ccc31)-c1cccc(c1)C[C@H](NC(=O)[C@H](C(C)C)N(C)C(=O)CCN=C=NCCOC)C(=O)N1CCC[C@H](N1)C(=O)OCC(C)(C)C2] |
| C24 A and B | 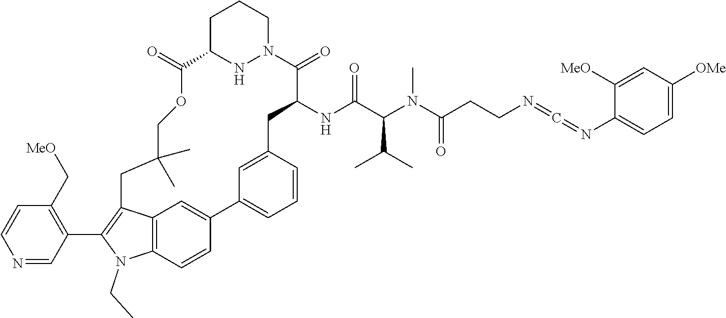 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1OC)C(=O)N1CCC[C@H](N1)C(=O)OCC(C)(C)C2] |
| C25 | 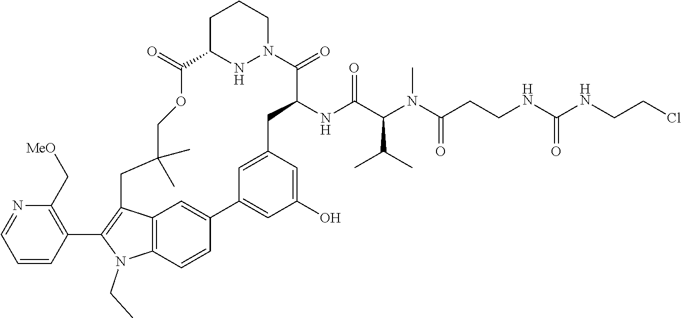 [SMILES: CCn1c(-c2cccnc2COC)c2c3cc(ccc31)-c1cc(O)cc(c1)C[C@H](NC(=O)[C@H](C(C)C)N(C)C(=O)CCNC(=O)NCCCl)C(=O)N1CCC[C@H](N1)C(=O)OCC(C)(C)C2] |
| C26 | 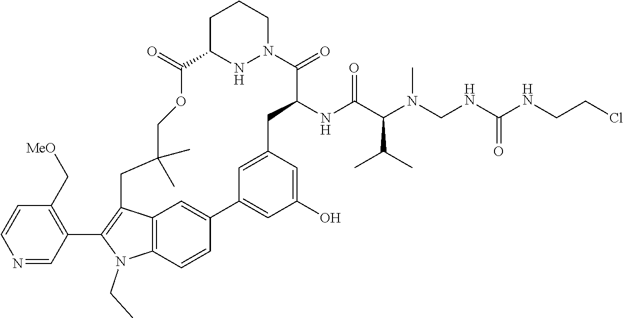 [SMILES: CCn1c(-c2cnccc2COC)c2c3cc(ccc31)-c1cc(O)cc(c1)C[C@H](NC(=O)[C@H](C(C)C)N(C)CNC(=O)NCCCl)C(=O)N1CCC[C@H](N1)C(=O)OCC(C)(C)C2] |
| C27 | 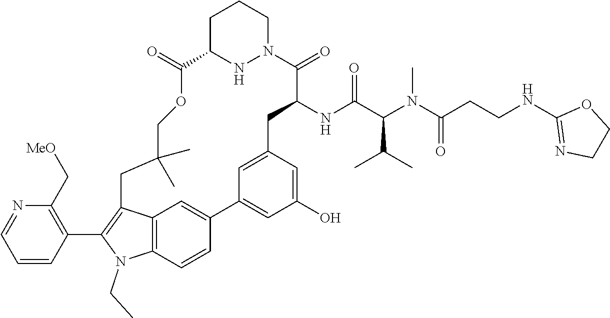 [SMILES: CCn1c(-c2cccnc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| C28 | 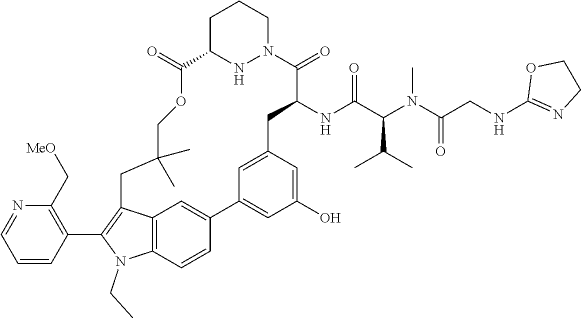 [SMILES: CCn1c(-c2cccnc2COC)c2c3cc(ccc31)-c1cc(O)cc(c1)C[C@H](NC(=O)[C@H](C(C)C)N(C)C(=O)CNC1=NCCO1)C(=O)N1CCC[C@H](N1)C(=O)OCC(C)(C)C2] |
| C29 | 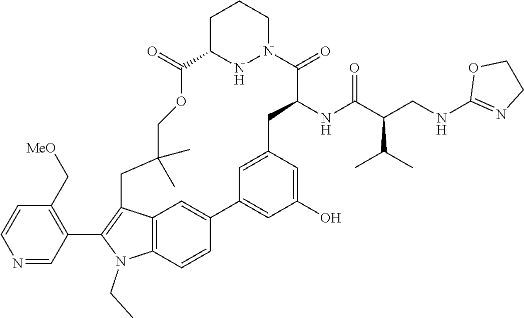 [SMILES: CCn1c(-c2cnccc2COC)c2c3cc(ccc31)-c1cc(O)cc(c1)C[C@H](NC(=O)[C@@H](CNC1=NCCO1)C(C)C)C(=O)N1CCC[C@H](N1)C(=O)OCC(C)(C)C2] |
| C30 | 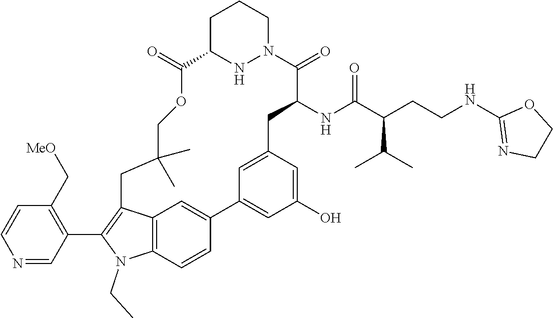 [SMILES: CCn1c(-c2cnccc2COC)c2c3cc(ccc31)-c1cc(O)cc(c1)C[C@H](NC(=O)[C@@H](CCNC1=NCCO1)C(C)C)C(=O)N1CCC[C@H](N1)C(=O)OCC(C)(C)C2] |
| C31 | 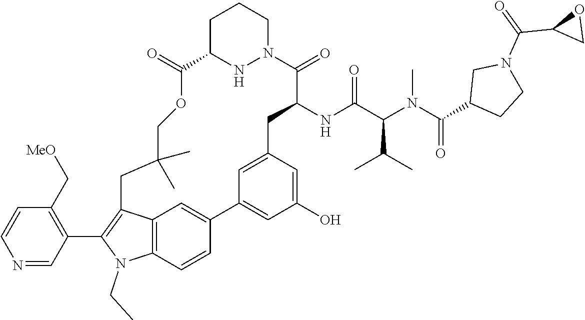 |
| C32 A and B | 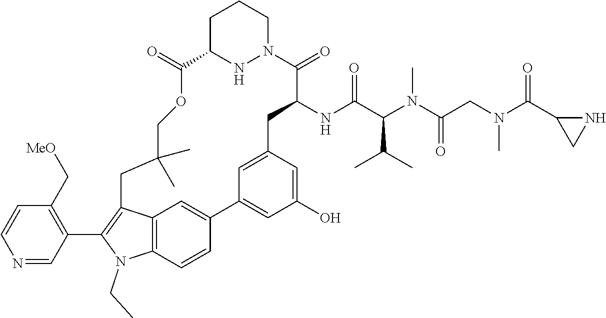 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| C33 | 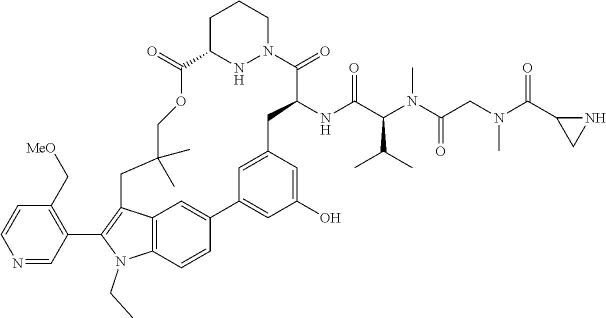 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| C34 | 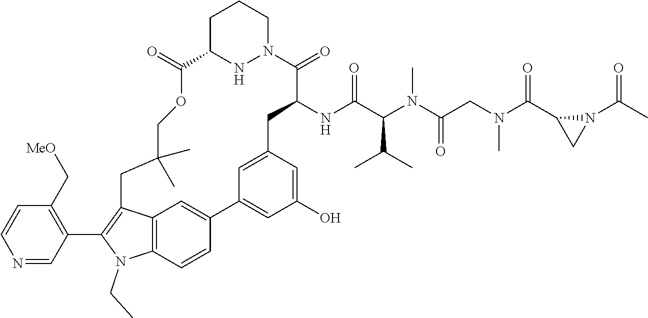 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(C)=O)C(=O)N1CCC[C@H](N1)C(=O)OCC(C)(C)C2] |
| C35 | 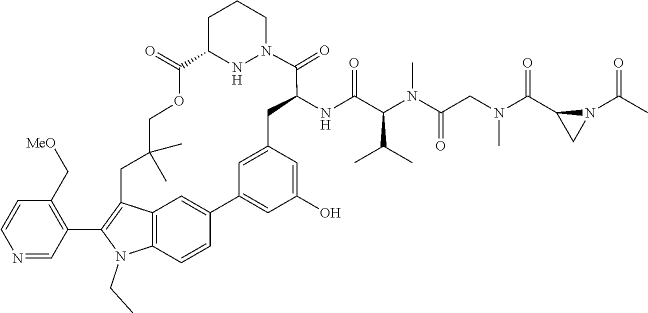 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(C)=O)C(=O)N1CCC[C@H](N1)C(=O)OCC(C)(C)C2] |
| C36 | 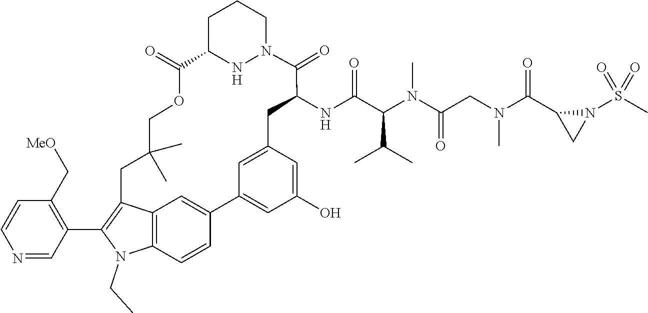 |
| C37 | 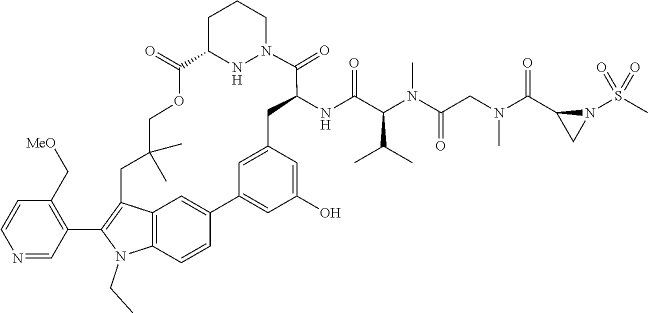 |
| C38 | 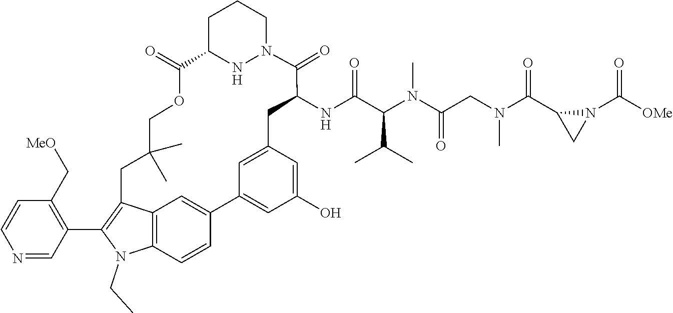 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(=O)OC)C(=O)N1CCC[C@H](N1)C(=O)OCC(C)(C)C2] |
| C39 | 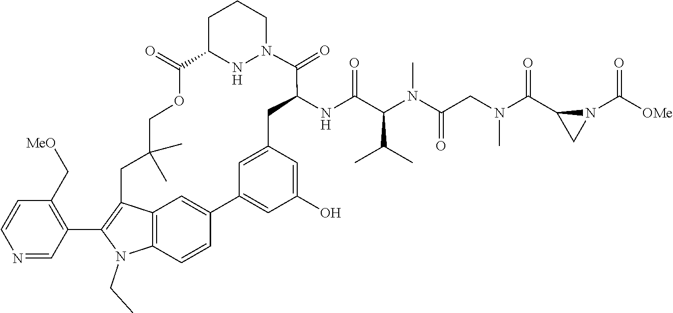 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(=O)OC)C(=O)N1CCC[C@H](N1)C(=O)OCC(C)(C)C2] |
| C40 | 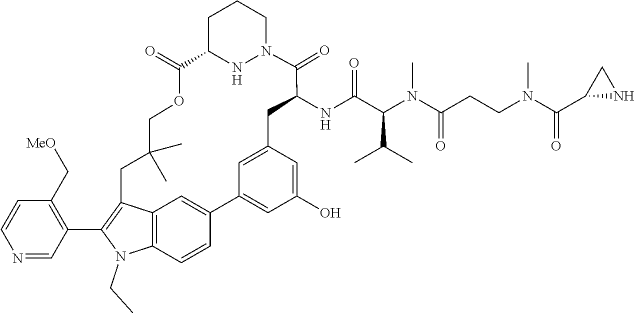 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@@H]1CN1)C(=O)N1CCC[C@H](N1)C(=O)OCC(C)(C)C2] |
| C41 | 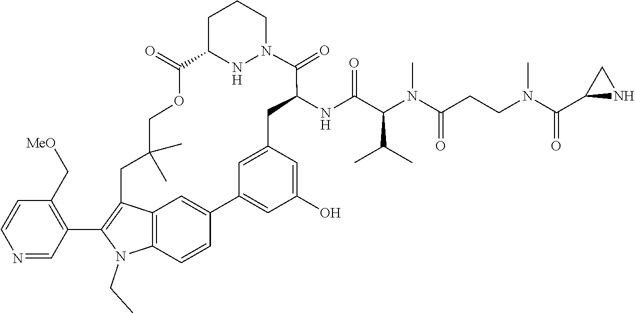 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@H]1CN1)C(=O)N1CCC[C@H](N1)C(=O)OCC(C)(C)C2] |
| C42A | 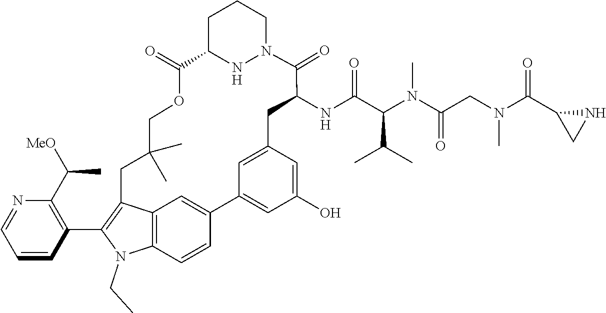 |
| C42B | 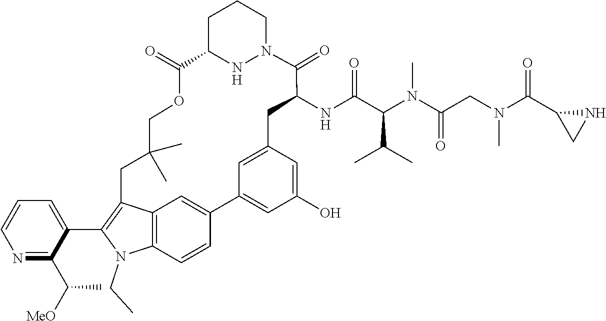 |
| C43A | 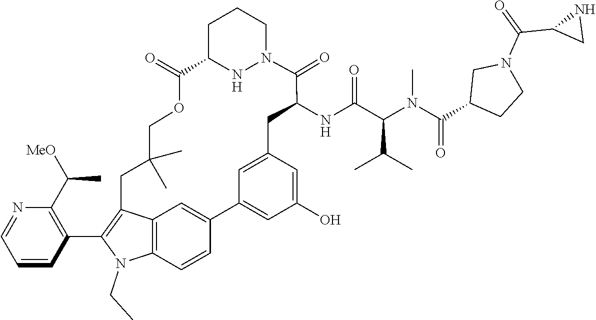 |
| C43B | 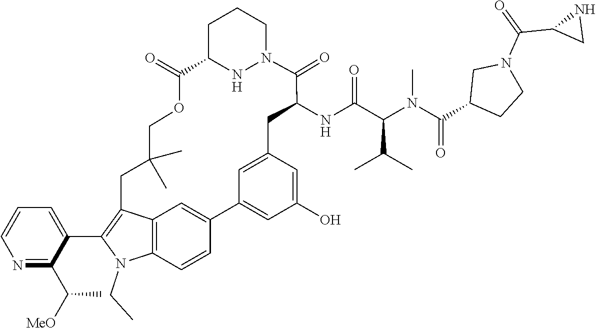 |
| C44 | 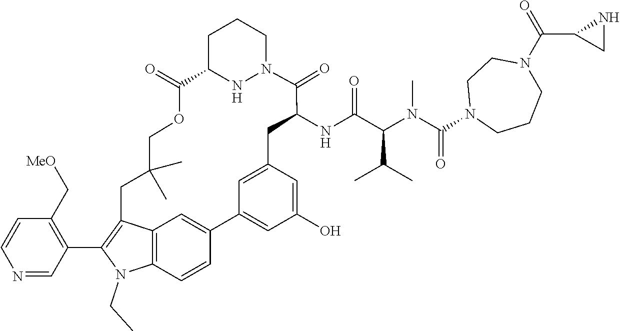 |
| C45 | 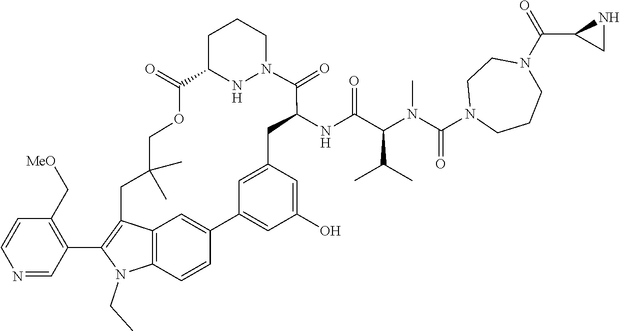 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@@H]3CN3)CC1)C(=O)N1CCC[C@H](N1)C(=O)OCC(C)(C)C2] |
| C46 | 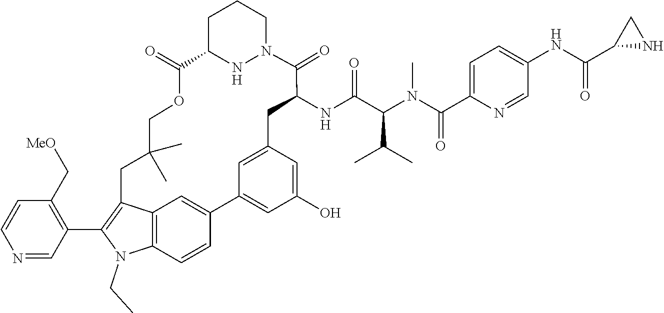 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3CN3)cn1)C(=O)N1CCC[C@H](N1)C(=O)OCC(C)(C)C2] |
| C47 A and B | 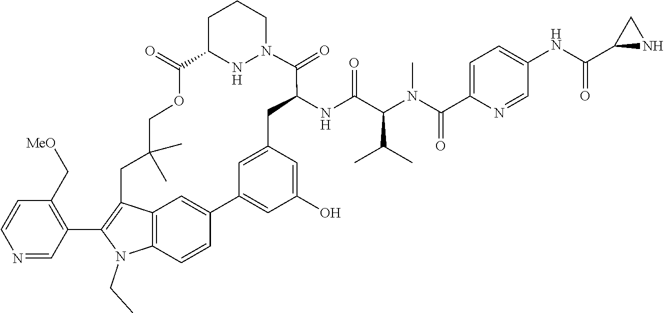 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3CN3)cn1)C(=O)N1CCC[C@H](N1)C(=O)OCC(C)(C)C2] |
| C48 | 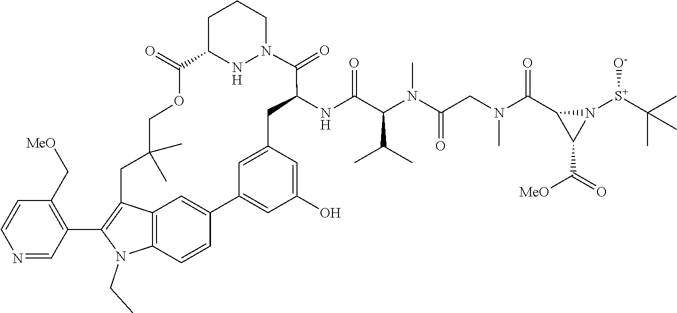 |
| C49 | 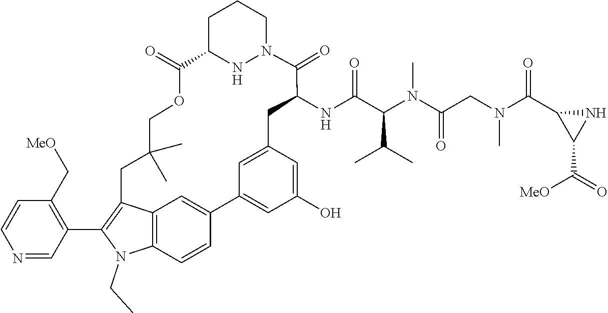 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C(=O)OC)C(=O)N1CCC[C@H](N1)C(=O)OCC(C)(C)C2] |
| C50 | 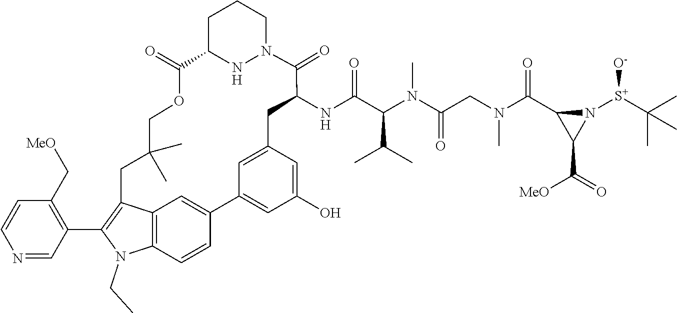 |
| C51 | 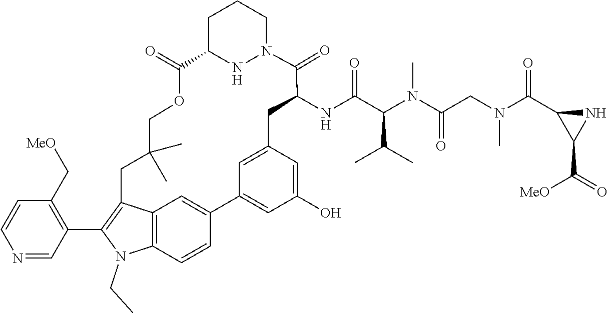 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C(=O)OC)C(=O)N1CCC[C@H](N1)C(=O)OCC(C)(C)C2] |
| C52 | 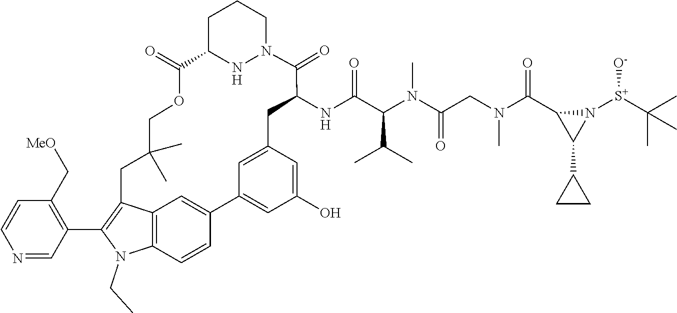 |
| C53 | 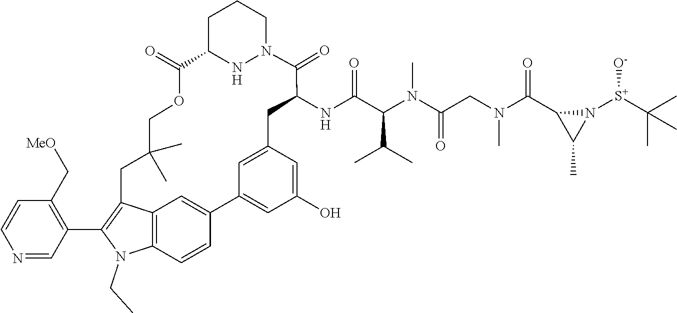 |
| C54 | 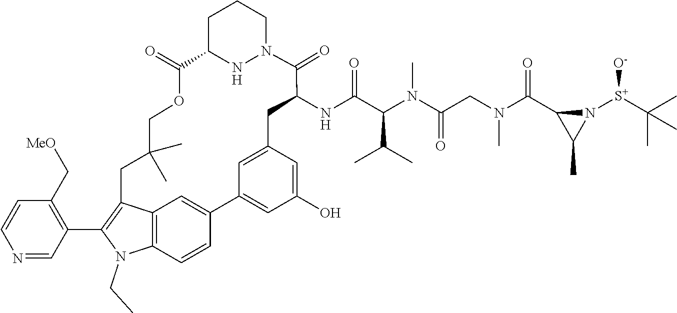 |
| C55 | 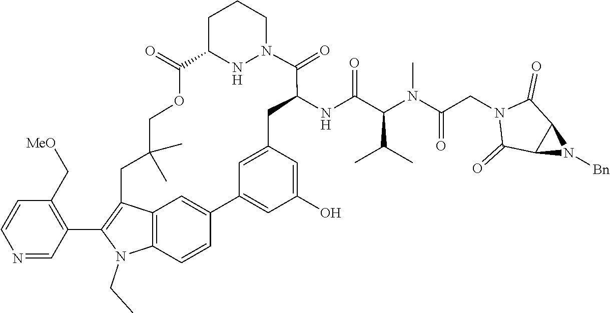 |
| C56* | 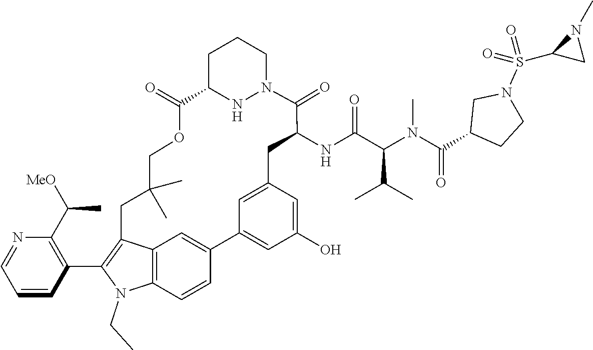 |
| C57* | 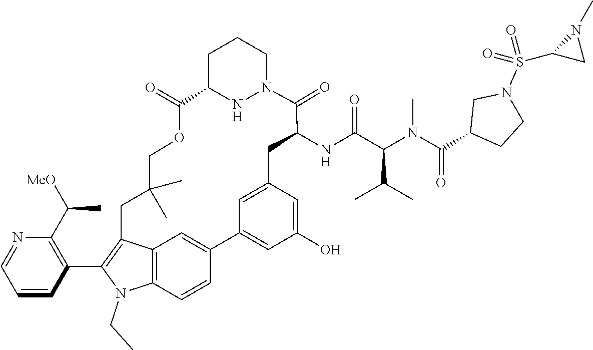 |
| C58 | 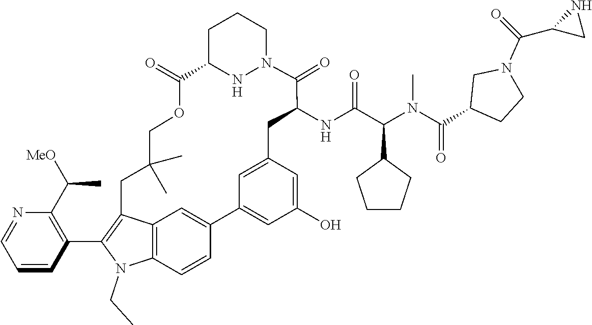 |
| C59 | 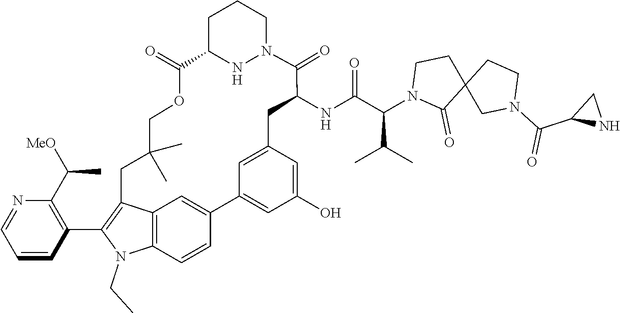 |
| C60 | 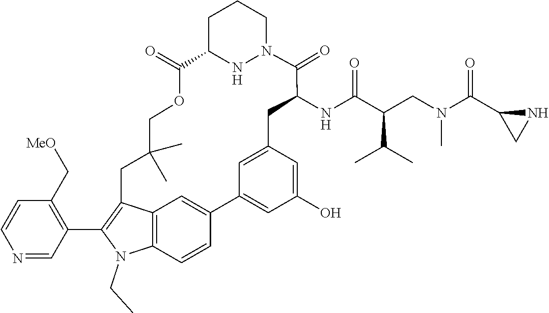 [SMILES: CCn1c(-c2cnccc2COC)c2c3cc(ccc31)-c1cc(O)cc(c1)C[C@H](NC(=O)[C@@H](CN(C)C(=O)[C@@H]1CN1)C(C)C)C(=O)N1CCC[C@H](N1)C(=O)OCC(C)(C)C2] |
| C61 A and B | 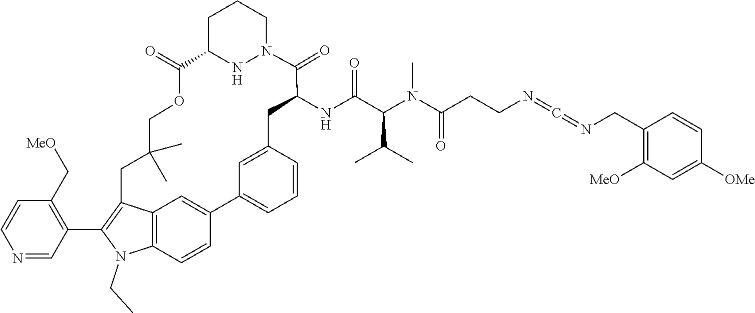 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1OC)C(=O)N1CCC[C@H](N1)C(=O)OCC(C)(C)C2] |
| C62 A and B | 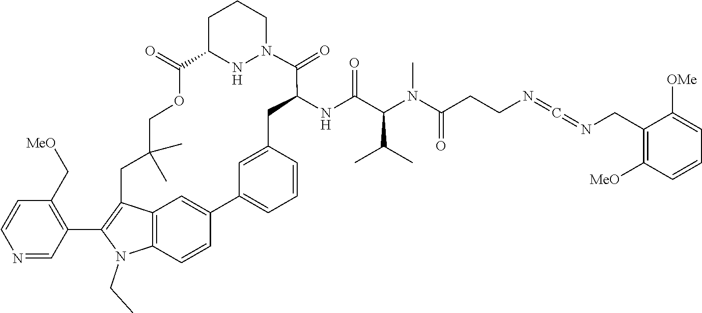 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| C63 | 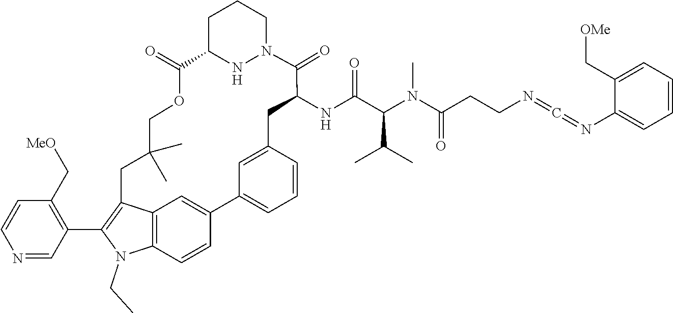 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1COC)C(=O)N1CCC[C@H](N1)C(=O)OCC(C)(C)C2] |
| C64 A and B | 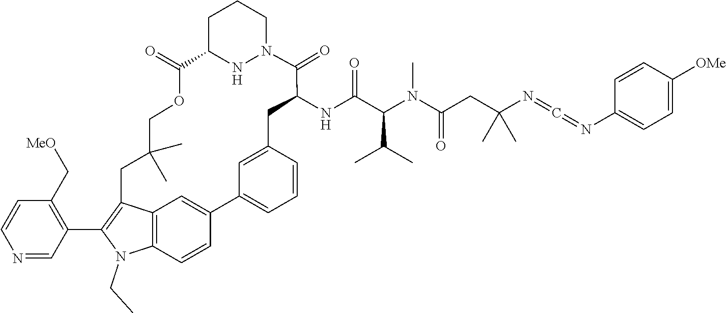 [SMILES: CCn1c(-c2cnccc2COC)c2c3cc(ccc31)-c1cccc(c1)C[C@H](NC(=O)[C@H](C(C)C)N(C)C(=O)CC(C)(C)N=C=Nc1ccc(OC)cc1)C(=O)N1CCC[C@H](N1)C(=O)OCC(C)(C)C2] |
| C65 A and B | 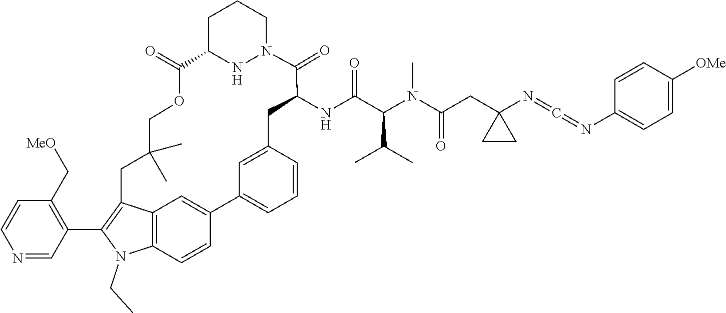 [SMILES: CCn1c(-c2cnccc2COC)c2c3cc(ccc31)-c1cccc(c1)C[C@H](NC(=O)[C@H](C(C)C)N(C)C(=O)CC1(N=C=Nc3ccc(OC)cc3)CC1)C(=O)N1CCC[C@H](N1)C(=O)OCC(C)(C)C2] |
| C66 | 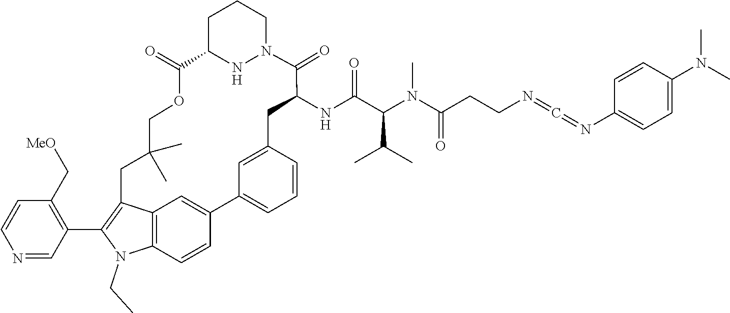 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N(C)C)cc1)C(=O)N1CCC[C@H](N1)C(=O)OCC(C)(C)C2] |
| C67 | 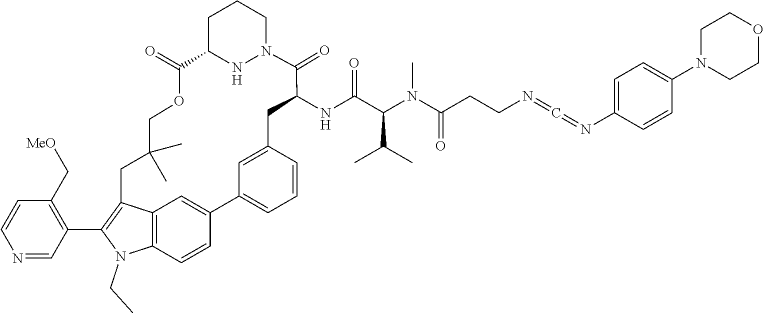 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(N3CCOCC3)cc1)C(=O)N1CCC[C@H](N1)C(=O)OCC(C)(C)C2] |
| C68 A and B | 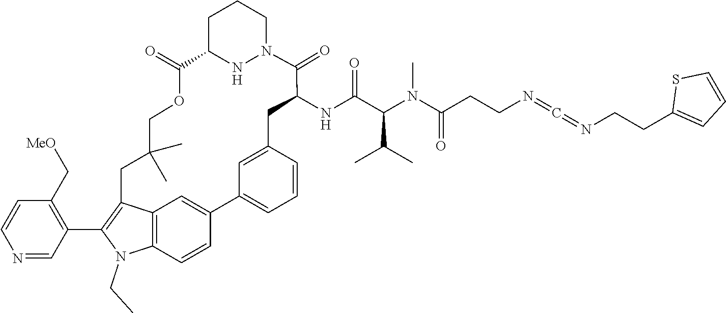 [SMILES: CCn1c(-c2cnccc2COC)c2c3cc(ccc31)-c1cccc(c1)C[C@H](NC(=O)[C@H](C(C)C)N(C)C(=O)CCN=C=NCCc1cccs1)C(=O)N1CCC[C@H](N1)C(=O)OCC(C)(C)C2] |
| C69 A and B | 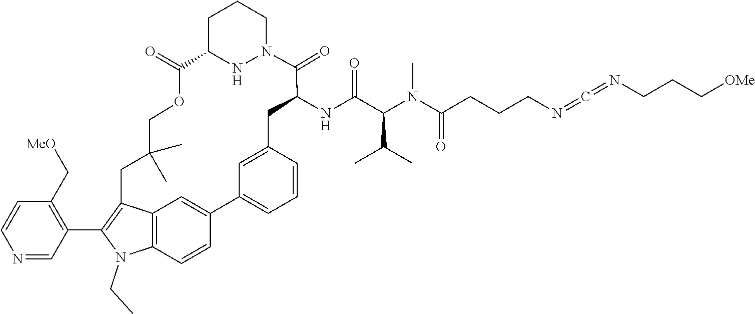 [SMILES: CCn1c(-c2cnccc2COC)c2c3cc(ccc31)-c1cccc(c1)C[C@H](NC(=O)[C@H](C(C)C)N(C)C(=O)CCCN=C=NCCCOC)C(=O)N1CCC[C@H](N1)C(=O)OCC(C)(C)C2] |
| C70 A and B | 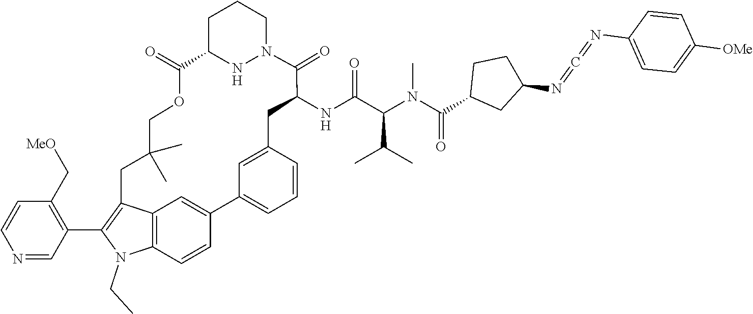 [SMILES: CCn1c(-c2cnccc2COC)c2c3cc(ccc31)-c1cccc(c1)C[C@H](NC(=O)[C@H](C(C)C)N(C)C(=O)[C@@H]1CC[C@@H](N=C=Nc3ccc(OC)cc3)C1)C(=O)N1CCC[C@H](N1)C(=O)OCC(C)(C)C2] |
| C71 A and B | 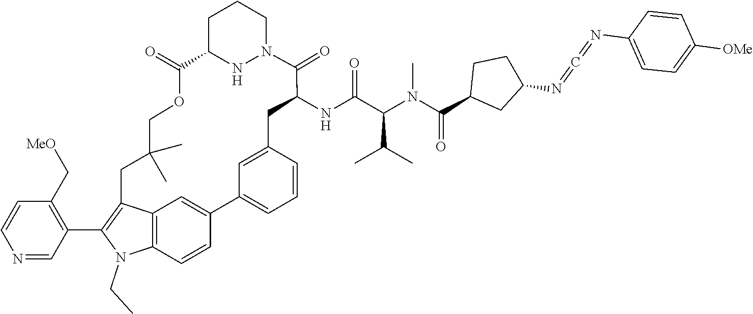 [SMILES: CCn1c(-c2cnccc2COC)c2c3cc(ccc31)-c1cccc(c1)C[C@H](NC(=O)[C@H](C(C)C)N(C)C(=O)[C@H]1CC[C@H](N=C=Nc3ccc(OC)cc3)C1)C(=O)N1CCC[C@H](N1)C(=O)OCC(C)(C)C2] |
| C72 A and B | 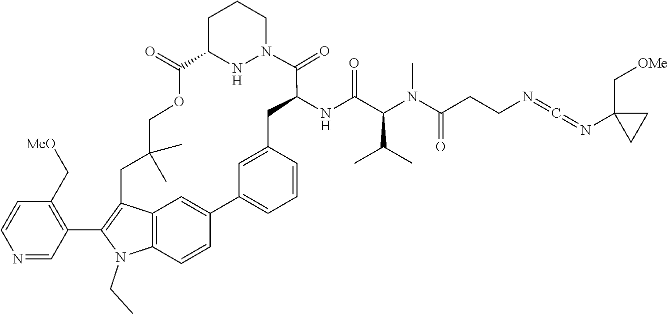 [SMILES: CCn1c(-c2cnccc2COC)c2c3cc(ccc31)-c1cccc(c1)C[C@H](NC(=O)[C@H](C(C)C)N(C)C(=O)CCN=C=NC1(COC)CC1)C(=O)N1CCC[C@H](N1)C(=O)OCC(C)(C)C2] |
| C73 | 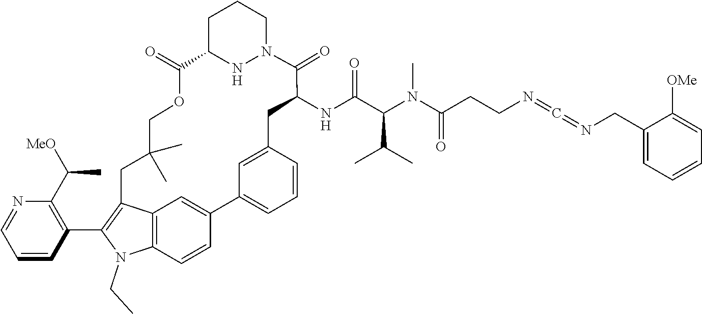 |
| C74 | 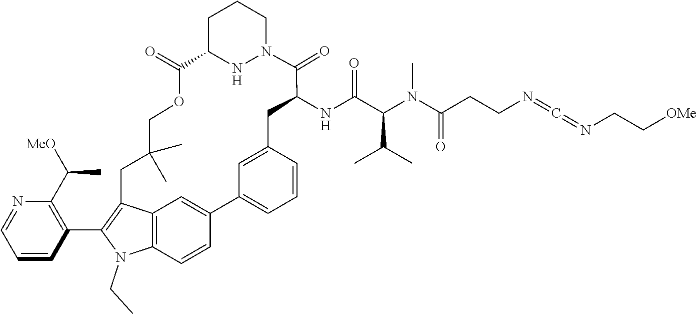 |
| C75 | 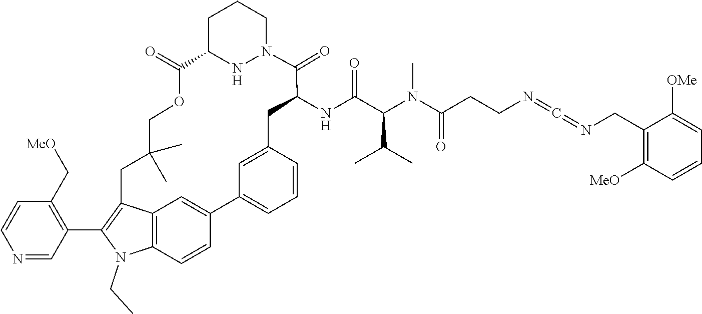 [SMILES: CCn1c(-c2cnccc2COC)c2c3cc(ccc31)-c1cccc(c1)C[C@H](NC(=O)[C@H](C(C)C)N(C)C(=O)CCN=C=NCc1c(OC)cccc1OC)C(=O)N1CCC[C@H](N1)C(=O)OCC(C)(C)C2] |
| C76 | 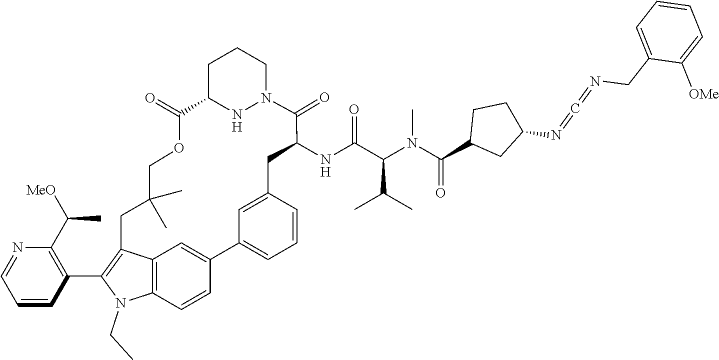 |
| C77 | 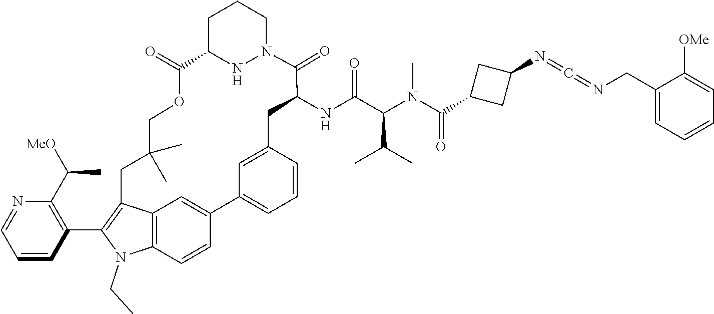 |
| C78 | 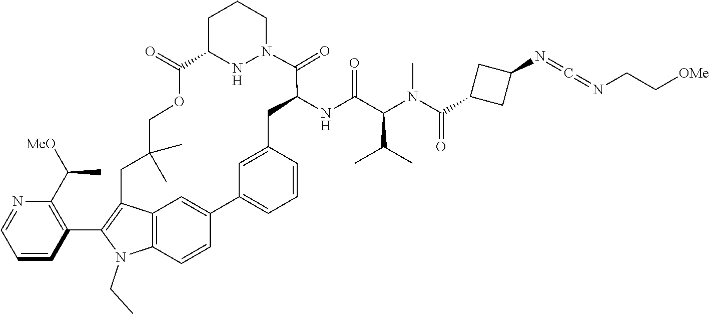 |
| C79 A and B | 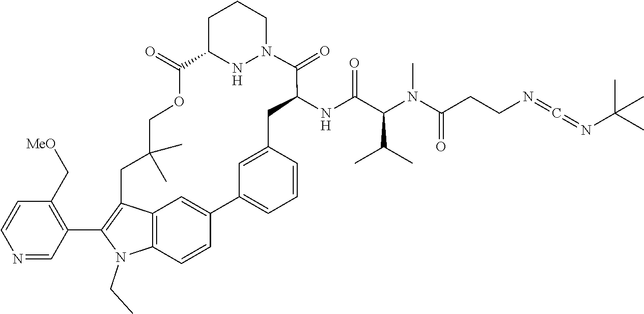 [SMILES: CCn1c(-c2cnccc2COC)c2c3cc(ccc31)-c1cccc(c1)C[C@H](NC(=O)[C@H](C(C)C)N(C)C(=O)CCN=C=NC(C)(C)C)C(=O)N1CCC[C@H](N1)C(=O)OCC(C)(C)C2] |
| C80 | 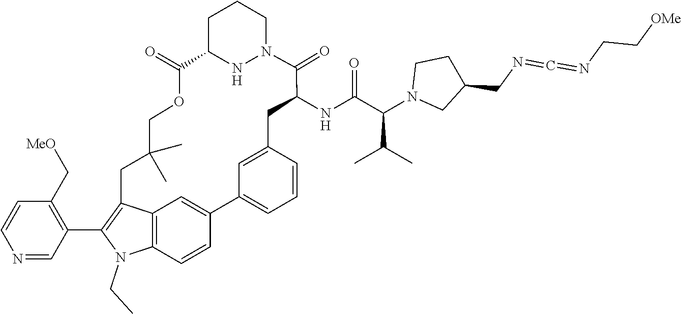 [SMILES: CCn1c(-c2cnccc2COC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| C81 | 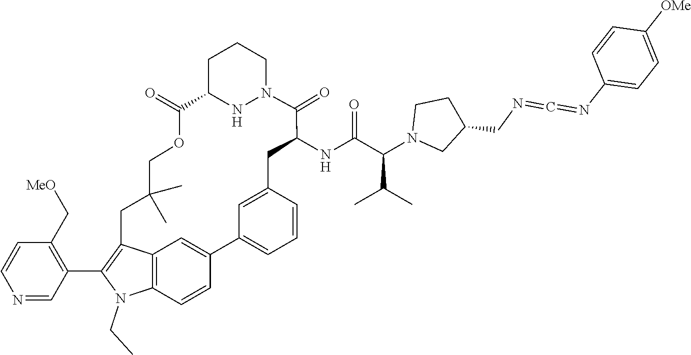 [SMILES: CCn1c(-c2cnccc2COC)c2c3cc(ccc31)-c1cccc(c1)C[C@H](NC(=O)[C@H](C(C)C)N1CC[C@H](CN=C=Nc3ccc(OC)cc3)C1)C(=O)N1CCC[C@H](N1)C(=O)OCC(C)(C)C2] |
| C82 A and B | 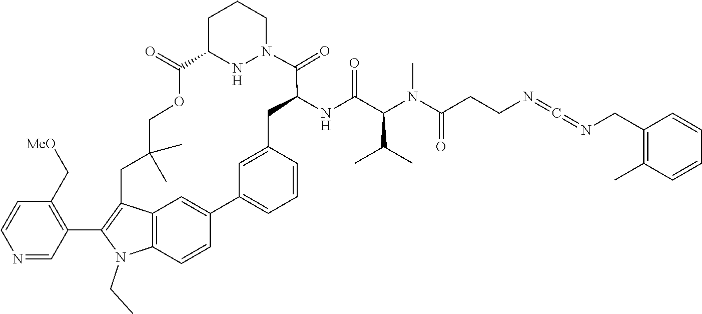 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1C)C(=O)N1CCC[C@H](N1)C(=O)OCC(C)(C)C2] |
| C83 | 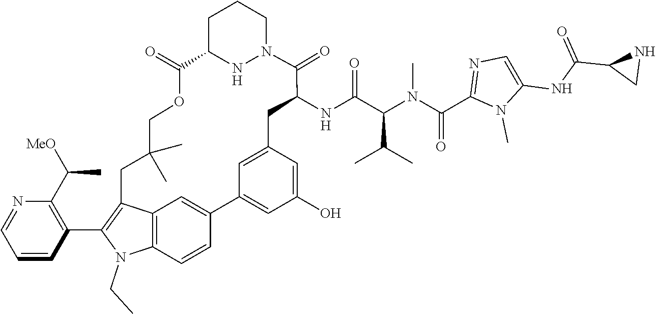 |
| C84 | 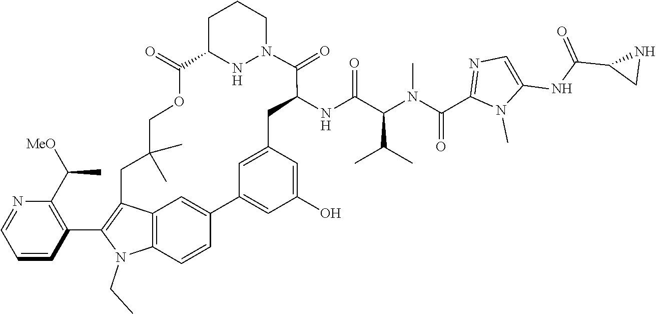 |
| C85 | 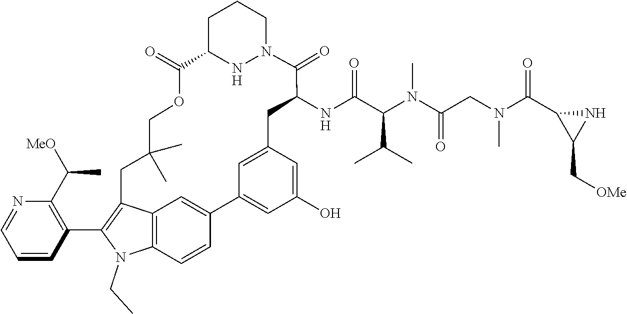 |
| C86 | 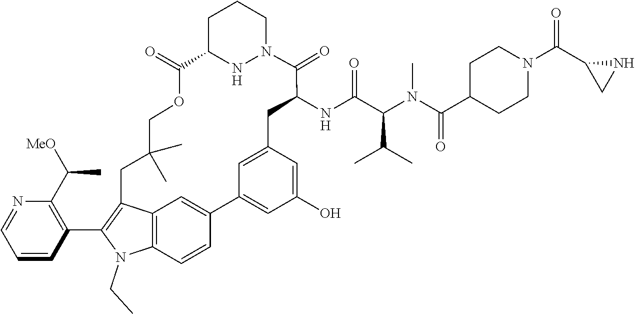 |
| C87 | 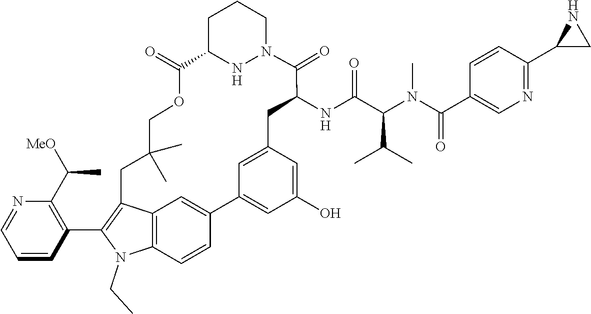 |
| C88 | 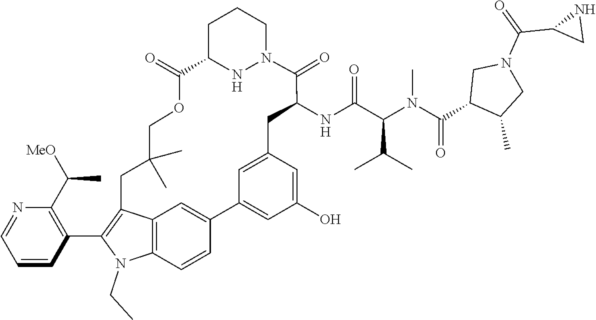 |
| C89 | 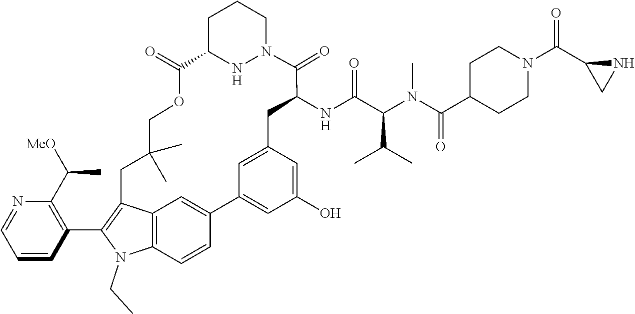 |
| C90 | 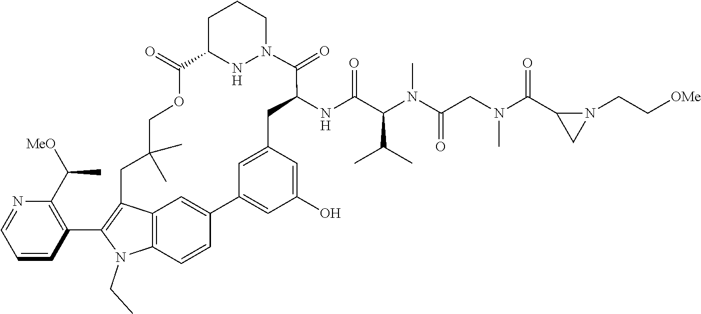 |
| C91* | 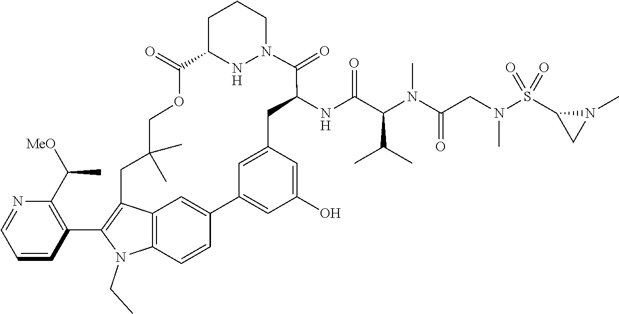 |
| C92* | 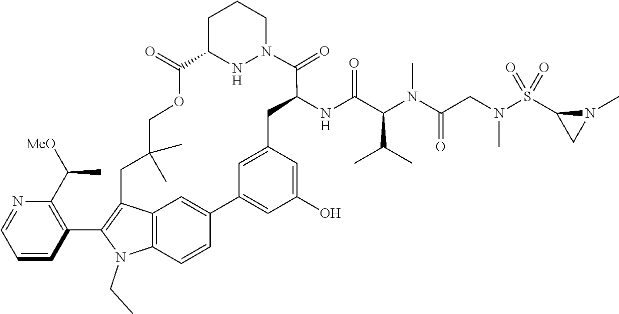 |
| C93 | 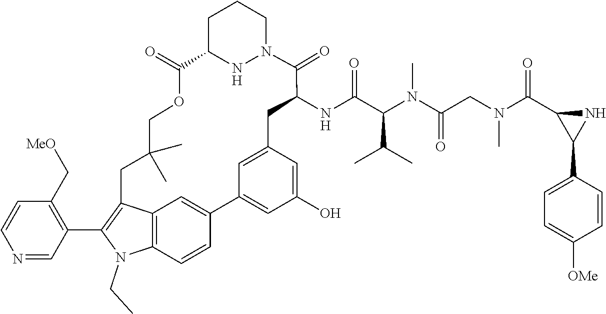 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccc(OC)cc1)C(=O)N1CCC[C@H](N1)C(=O)OCC(C)(C)C2] |
| C94 | 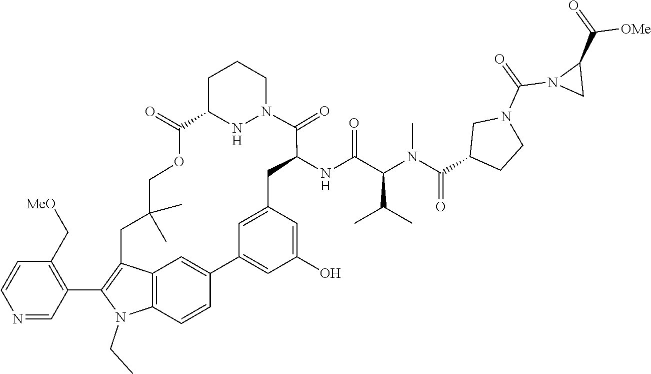 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@@H]3C(=O)OC)C1)C(=O)N1CCC[C@H](N1)C(=O)OCC(C)(C)C2] |
| C95 | 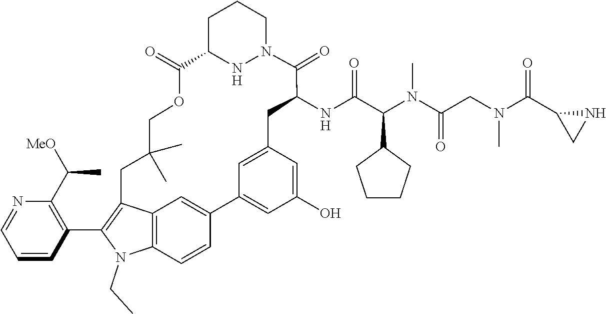 |
| C96 | 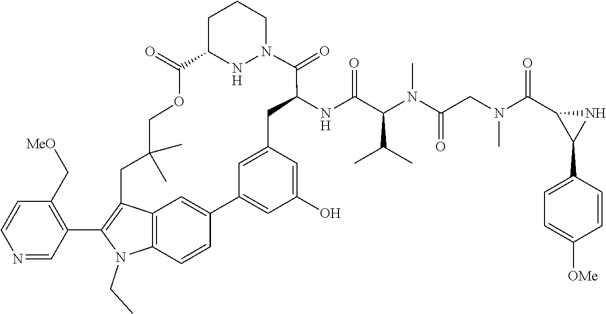 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccc(OC)cc1)C(=O)N1CCC[C@H](N1)C(=O)OCC(C)(C)C2] |
| C97 | 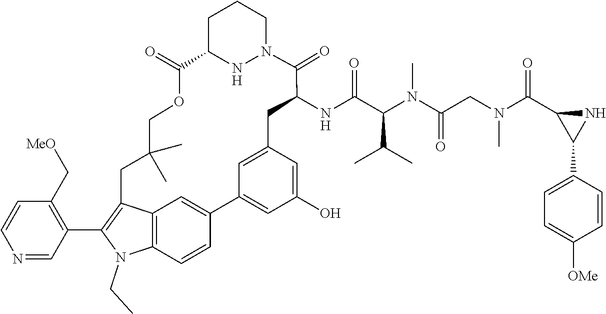 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccc(OC)cc1)C(=O)N1CCC[C@H](N1)C(=O)OCC(C)(C)C2] |
| C98 | 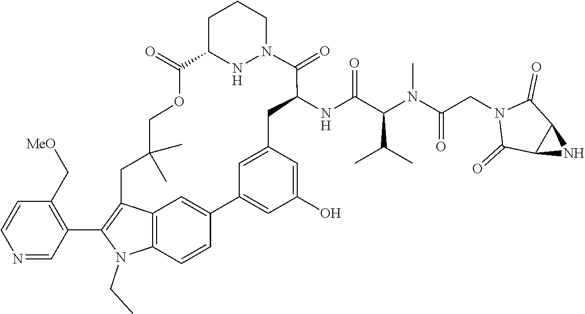 [SMILES: CCn1c(-c2cnccc2COC)c2c3cc(ccc31)-c1cc(O)cc(c1)C[C@H](NC(=O)[C@H](C(C)C)N(C)C(=O)CN1C(=O)[C@H]3N[C@H]3C1=O)C(=O)N1CCC[C@H](N1)C(=O)OCC(C)(C)C2] |
| C99 | 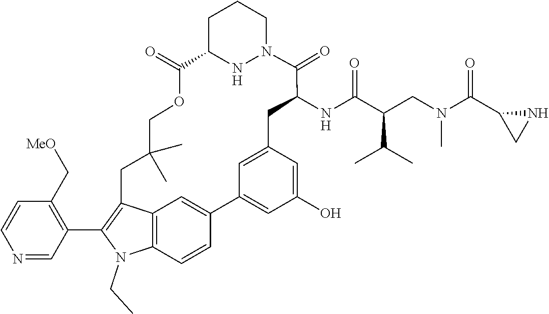 [SMILES: CCn1c(-c2cnccc2COC)c2c3cc(ccc31)-c1cc(O)cc(c1)C[C@H](NC(=O)[C@@H](CN(C)C(=O)[C@H]1CN1)C(C)C)C(=O)N1CCC[C@H](N1)C(=O)OCC(C)(C)C2] |
| C100 | 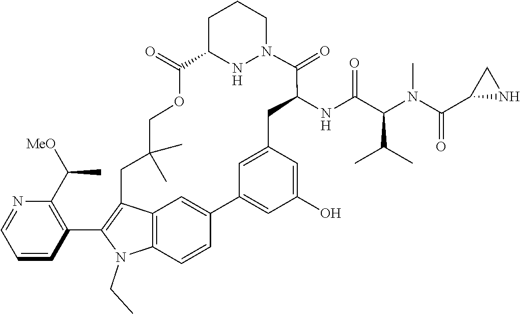 |
| C101 | 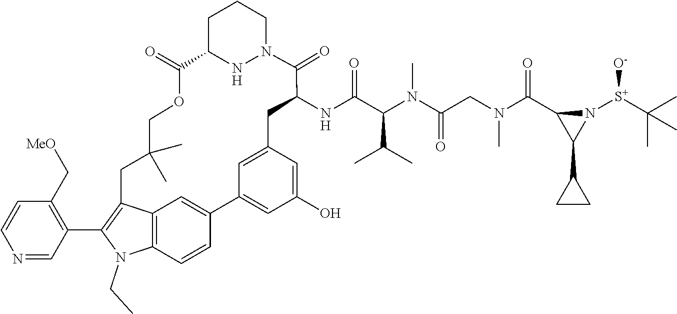 |
| C102 | 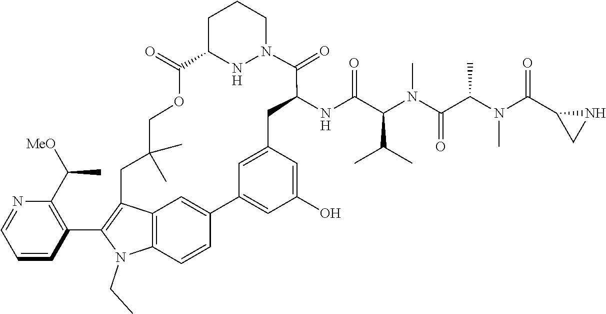 |
| C103 | 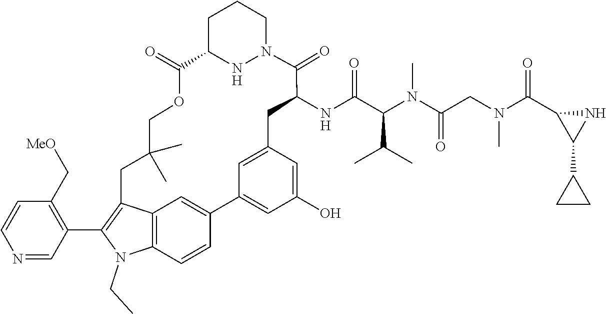 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C1CC1)C(=O)N1CCC[C@H](N1)C(=O)OCC(C)(C)C2] |
| C104 | 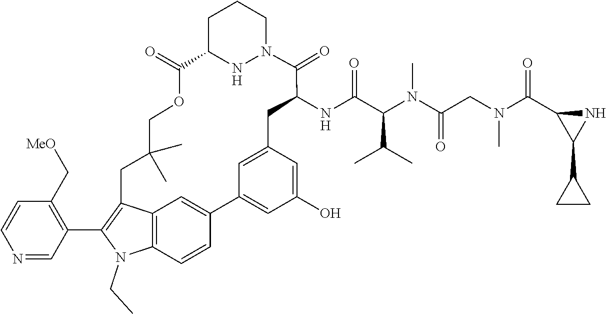 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C1CC1)C(=O)N1CCC[C@H](N1)C(=O)OCC(C)(C)C2] |
| C105 | 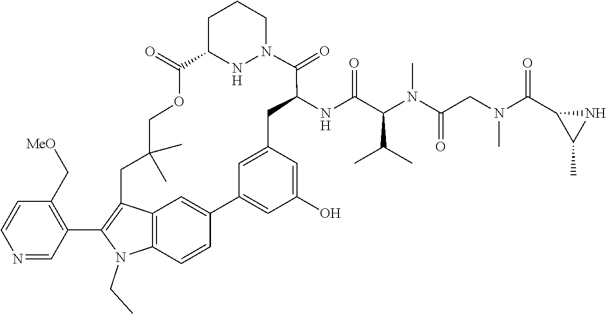 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C)C(=O)N1CCC[C@H](N1)C(=O)OCC(C)(C)C2] |
| C106 | 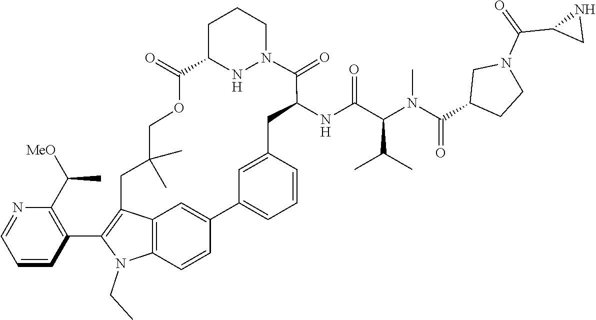 |
| C107 | 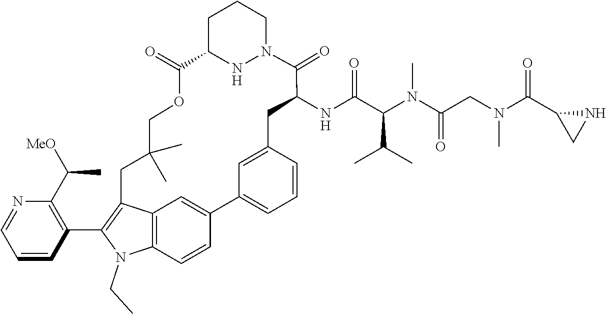 |
| C108* | 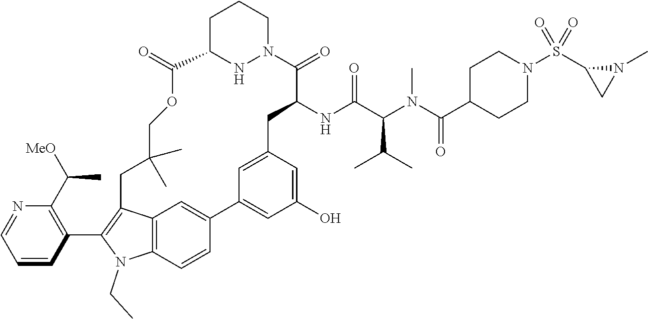 |
| C109 | 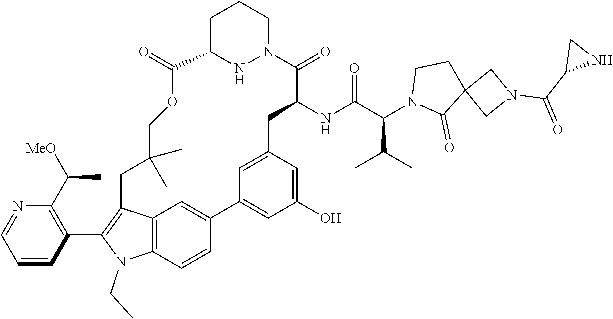 |
| C110 | 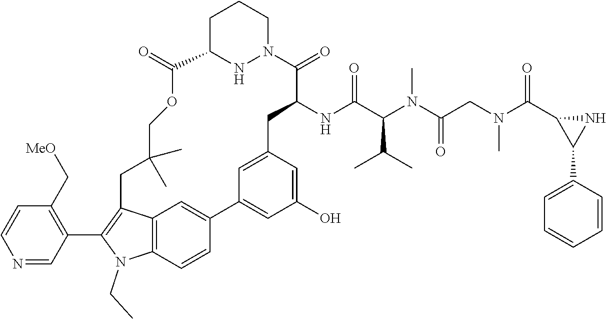 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccccc1)C(=O)N1CCC[C@H](N1)C(=O)OCC(C)(C)C2] |
| C111 | 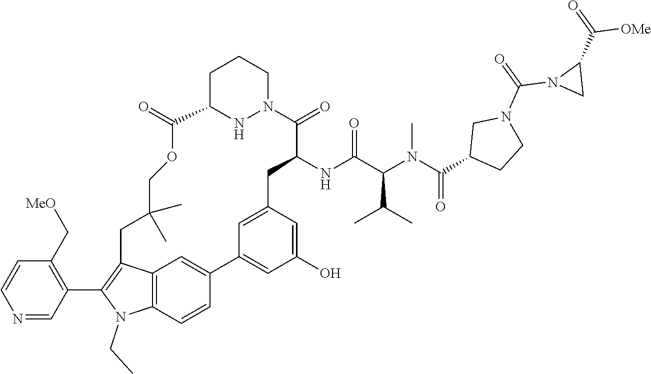 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@H]3C(=O)OC)C1)C(=O)N1CCC[C@H](N1)C(=O)OCC(C)(C)C2] |
| C112 | 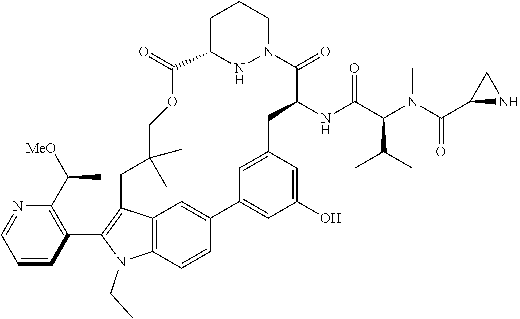 |
| C113 | 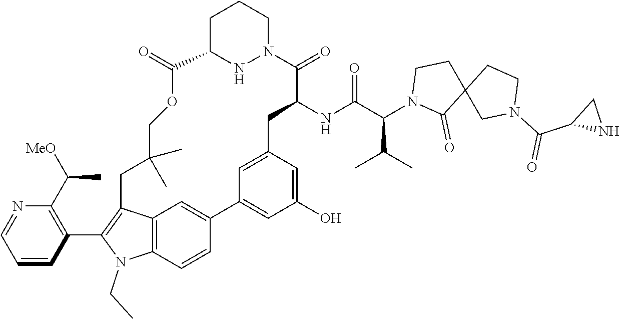 |
| C114 | 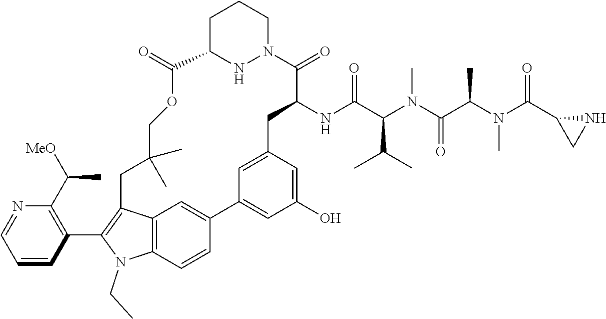 |
| C115 | 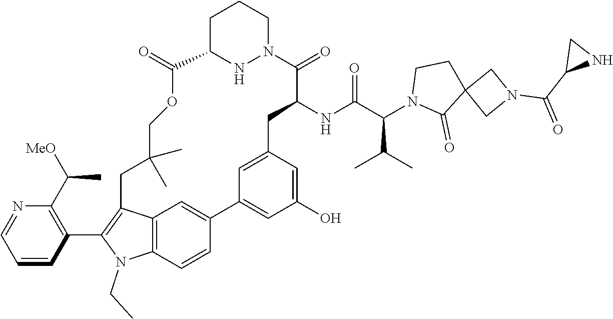 |
| C116 | 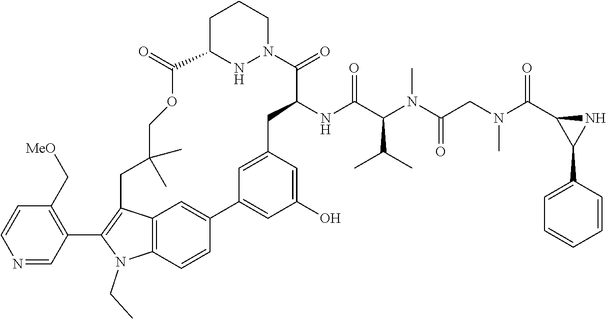 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccccc1)C(=O)N1CCC[C@H](N1)C(=O)OCC(C)(C)C2] |
| C117* | 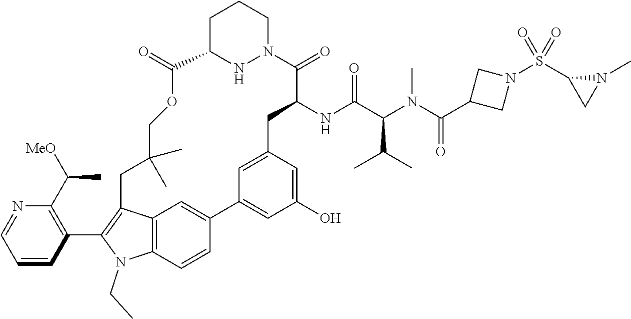 |
| C118* | 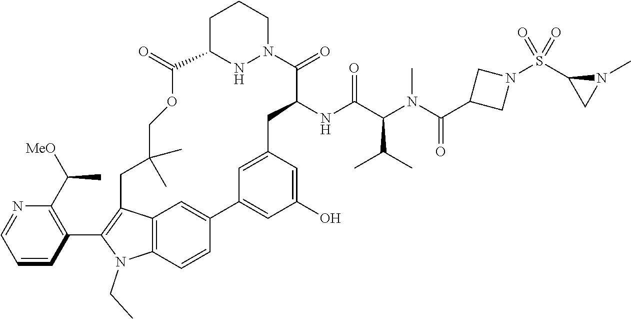 |
| C119* | 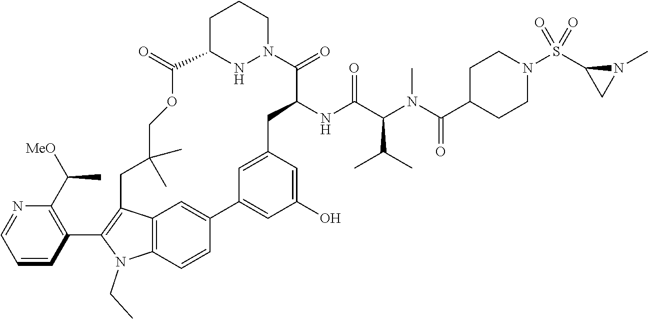 |
| C120 | 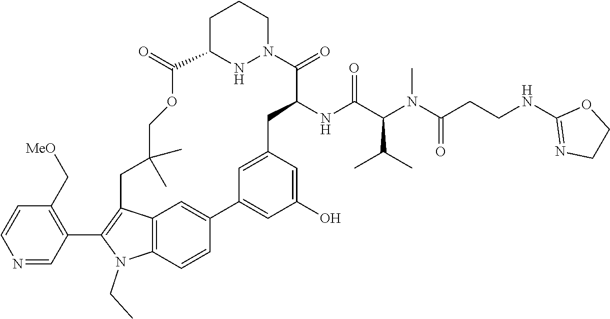 [SMILES: CCn1c(-c2cnccc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| C121 | 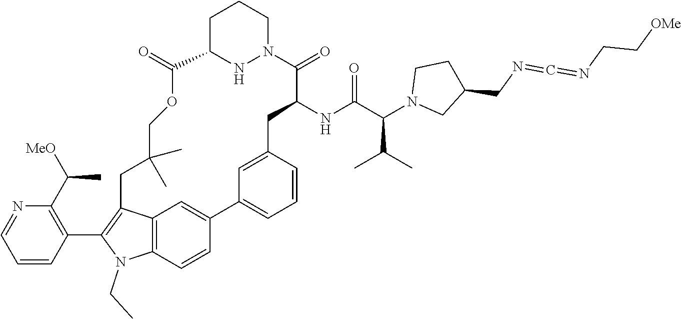 [SMILES: CCn1c(-c2cccnc2[C@H](C)OC)c2c3cc(ccc31)-c1cccc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| C122 | 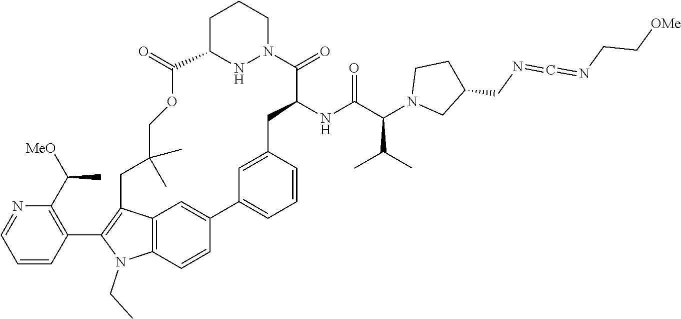 [SMILES: CCn1c(-c2cccnc2[C@H](C)OC)c2c3cc(ccc31)-c1cccc(c1)C[C@H](NC(=O)[C@H](C(C)C)N1CC[C@H](CN=C=NCCOC)C1)C(=O)N1CCC[C@H](N1)C(=O)OCC(C)(C)C2] |
| C123 | 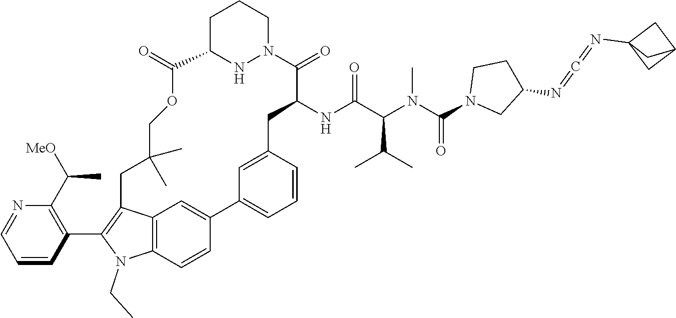 |
| C124 | 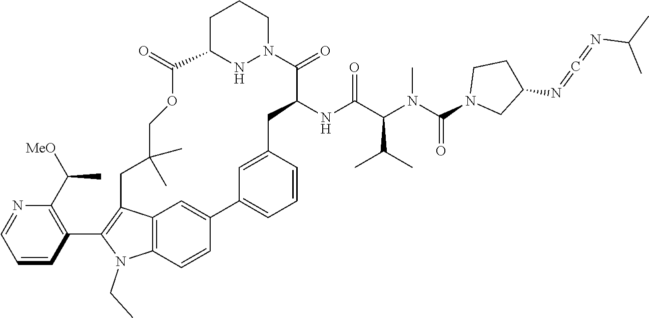 |
| C125 | 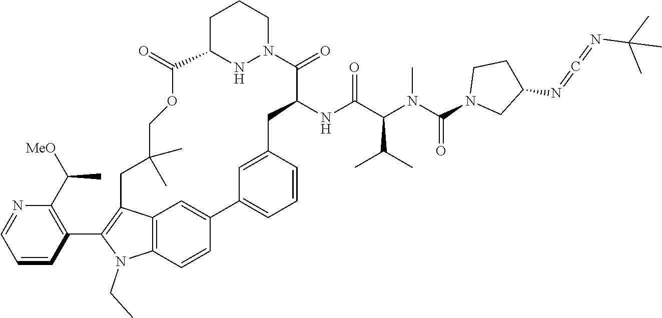 |
| C126 | 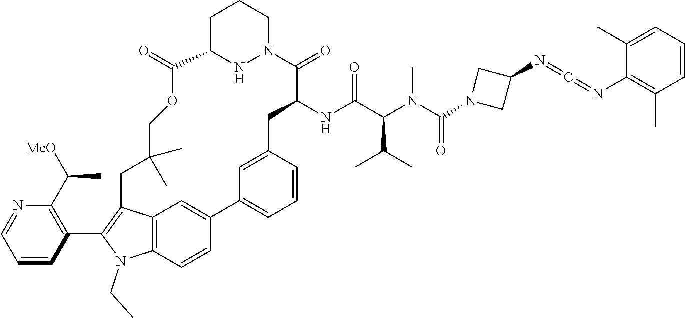 |
| C127 | 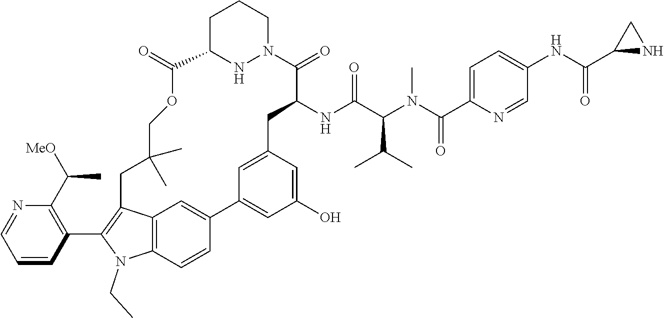 |
| C128 | 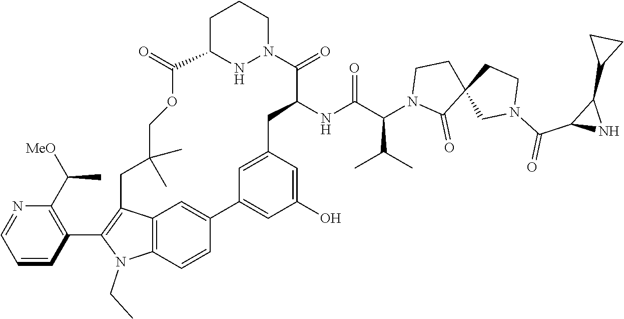 |
| C129 | 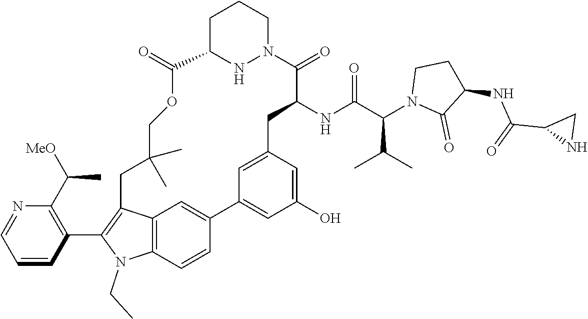 |
| C130 | 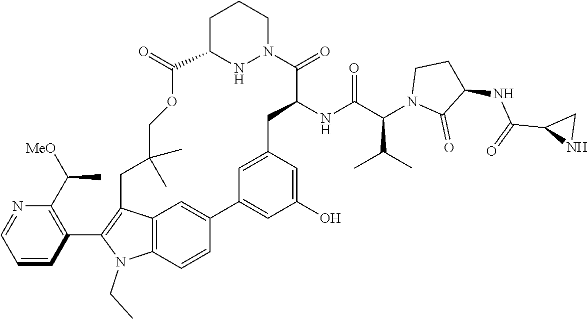 |
| C131 | 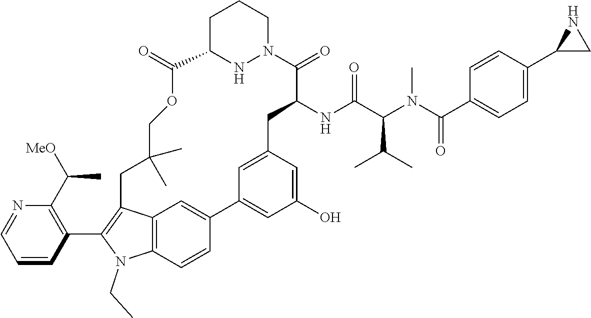 |
| C132 | 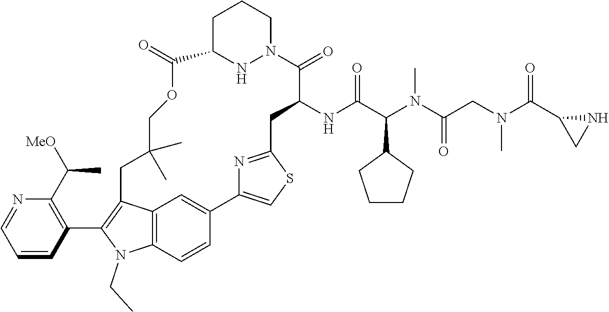 |
| C133 | 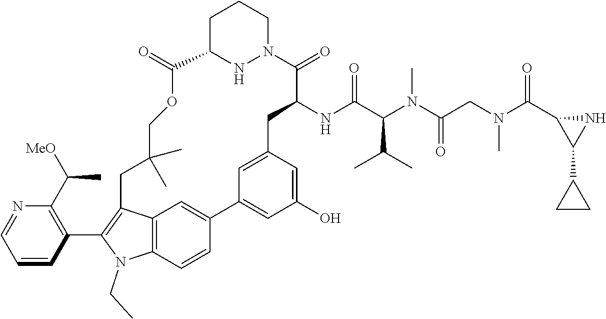 |
| C134 | 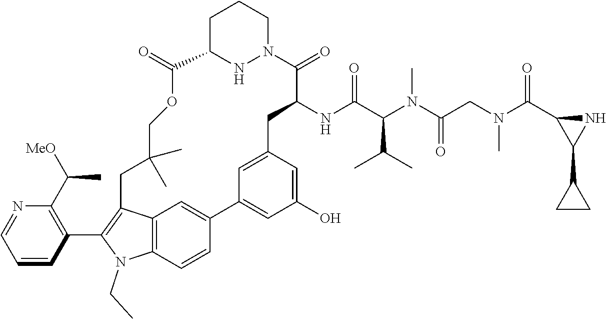 |
| C135 | 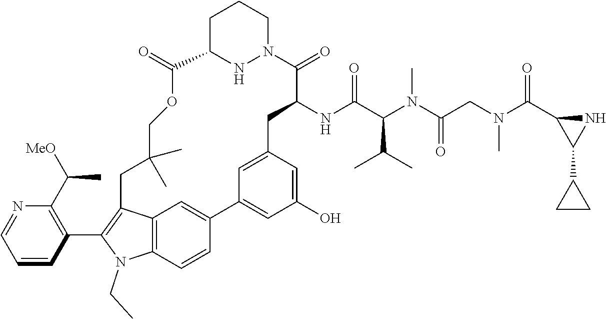 |
| C136 | 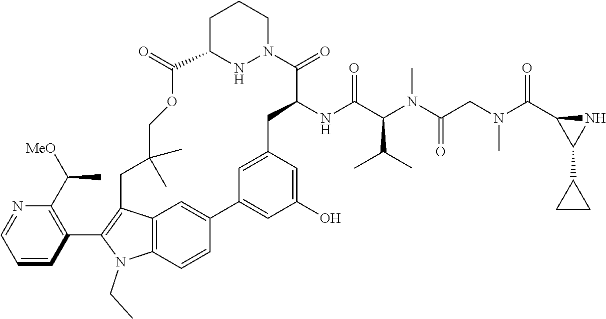 |
| C137 | 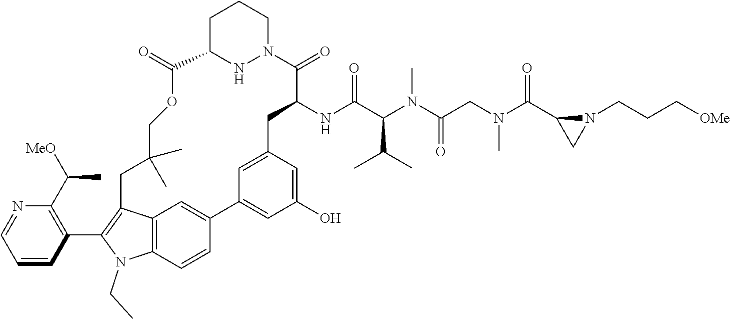 |
| C138 | 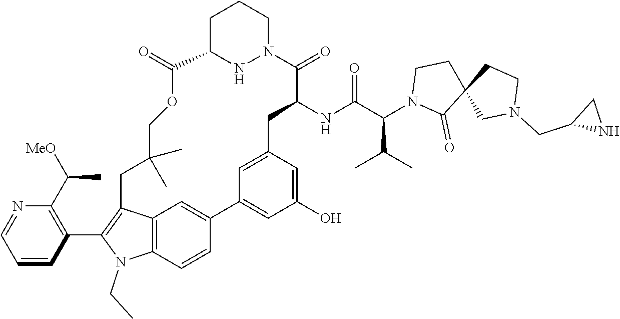 |
| C139 | 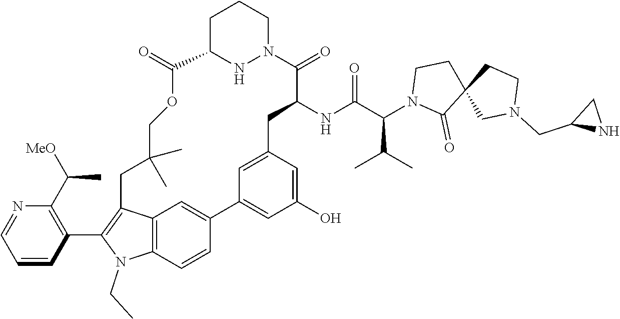 |
| C140 | 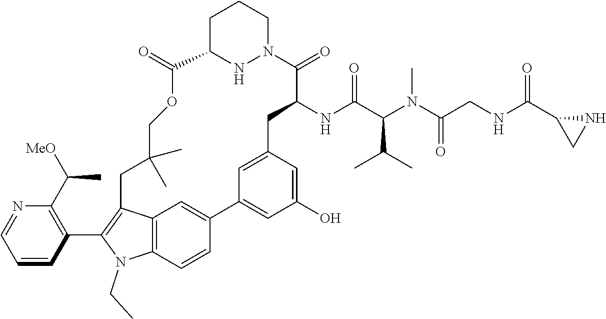 |
| C141 | 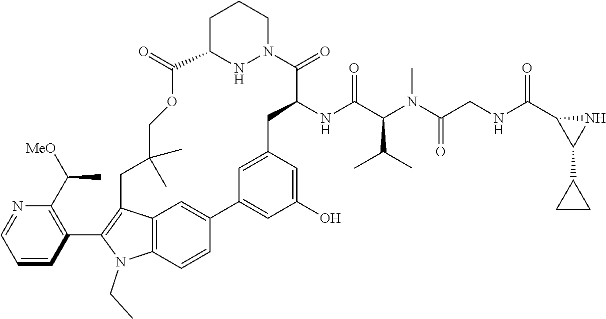 |
| C142 | 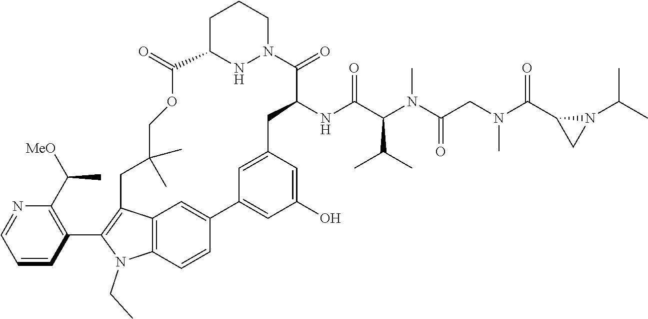 |
| C143 | 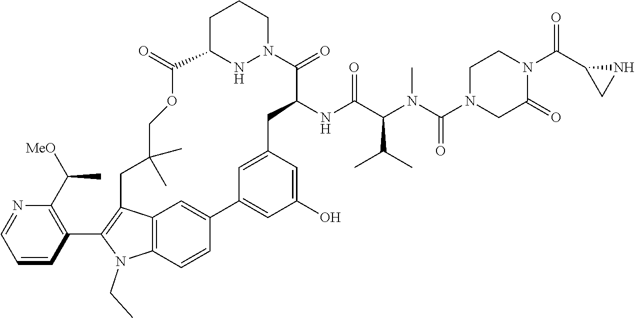 |
| C144 | 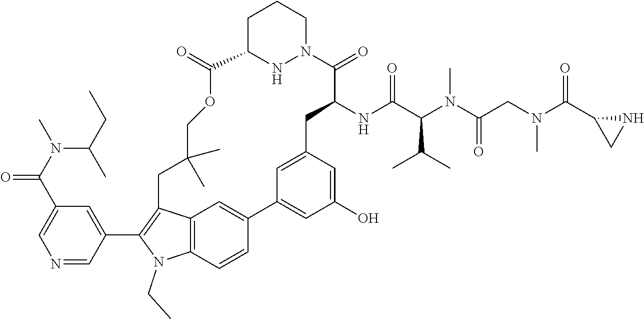 [SMILES: CCC(C)N(C)C(=O)c1cncc(-c2c3c4cc(ccc4n2CC)-c2cc(O)cc(c2)C[C@H](NC(=O)[C@H](C(C)C)N(C)C(=O)CN(C)C(=O)[C@H]2CN2)C(=O)N2CCC[C@H](N2)C(=O)OCC(C)(C)C3)c1] |
| C145 | 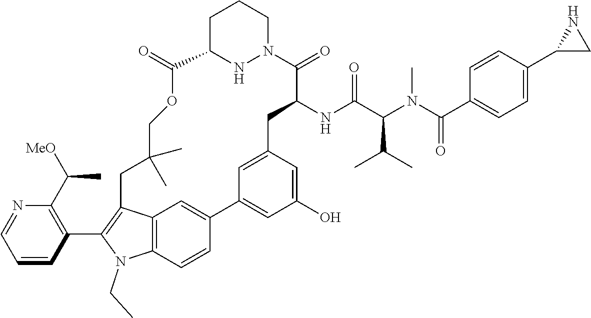 |
| C146 | 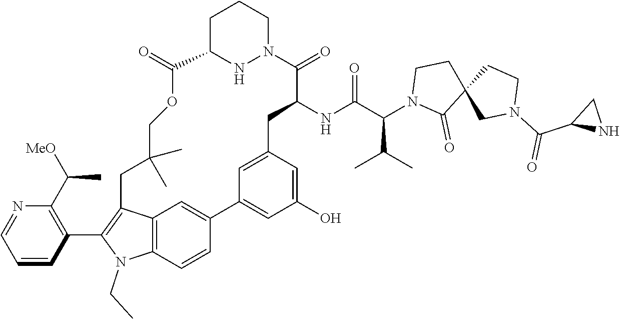 |
| C147 | 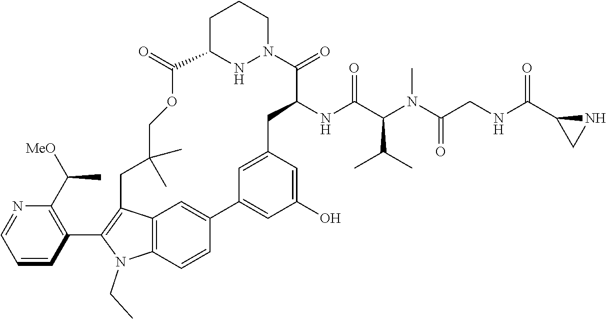 |
| C148 | 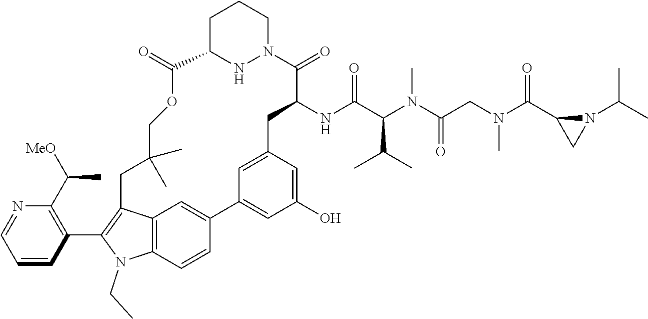 |
| C149 | 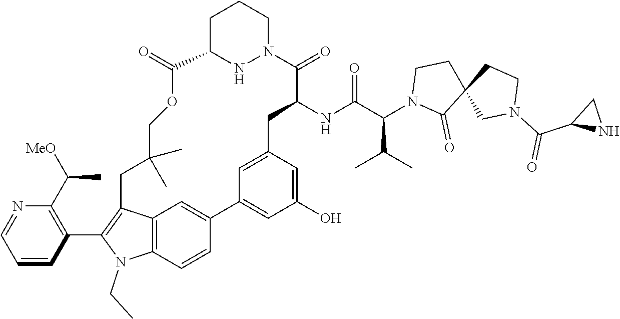 |
| C150 | 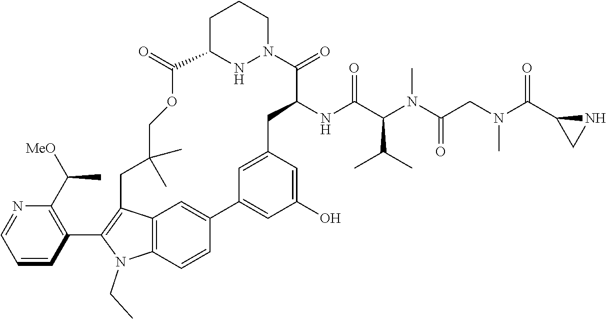 |
| C151 | 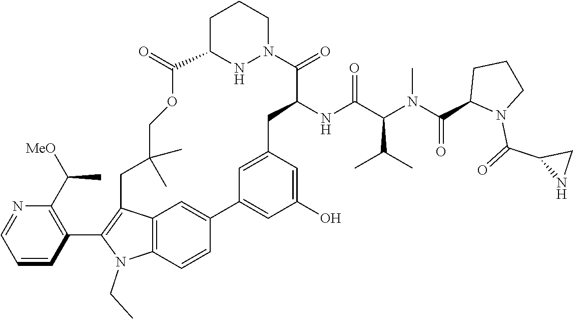 |
| C152 | 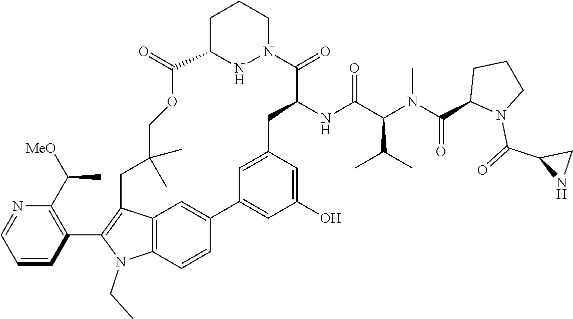 |
| C153 | 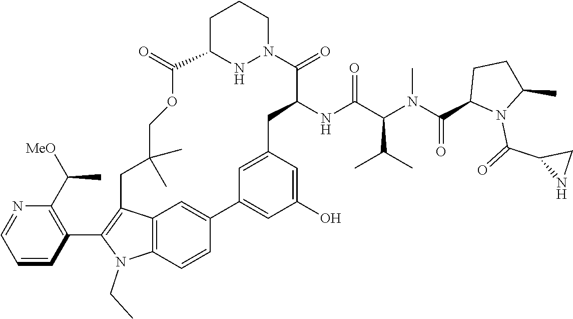 |
| C154 | 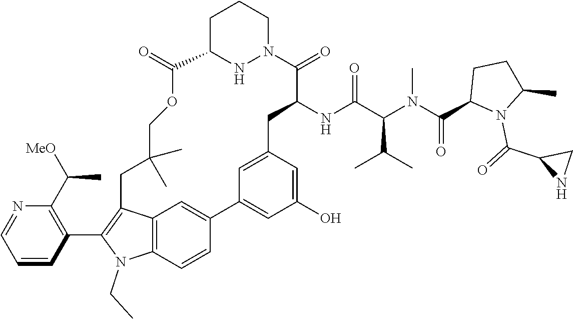 |
| C155 | 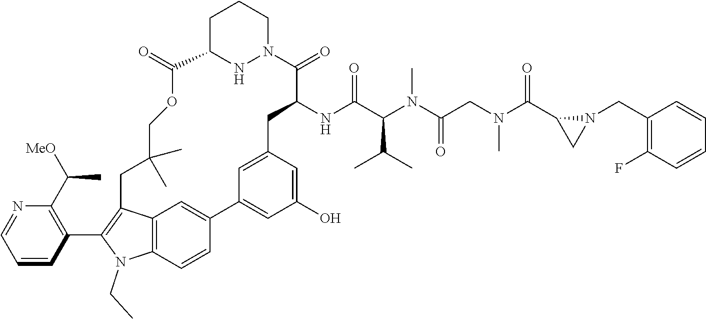 |
| C156 | 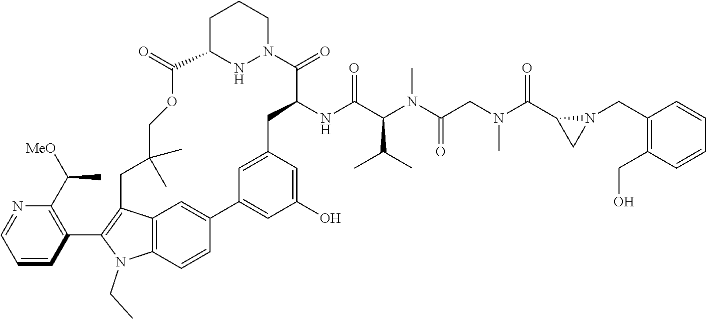 |
| C157 | 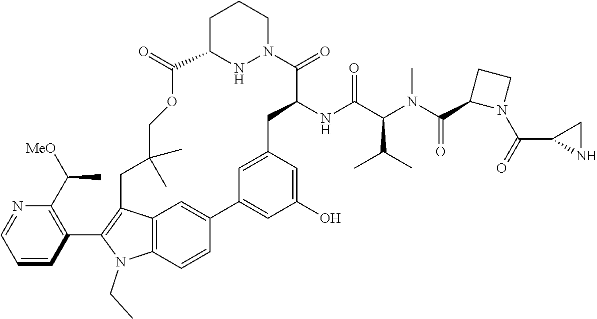 |
| C158 | 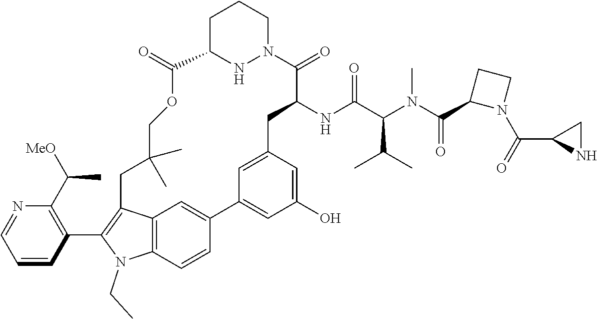 |
| C159 | 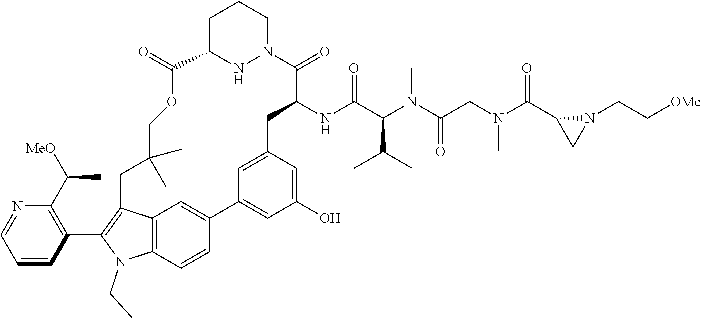 |
| C160 | 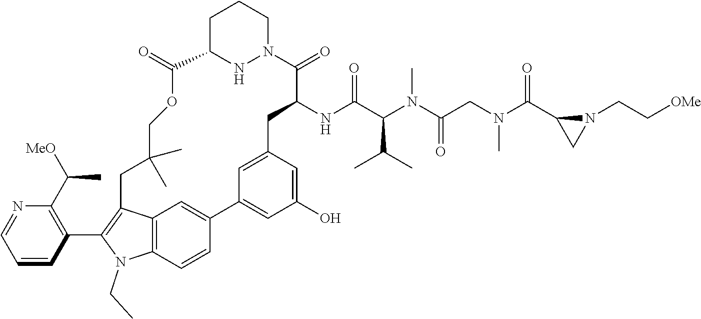 |
| C161 | 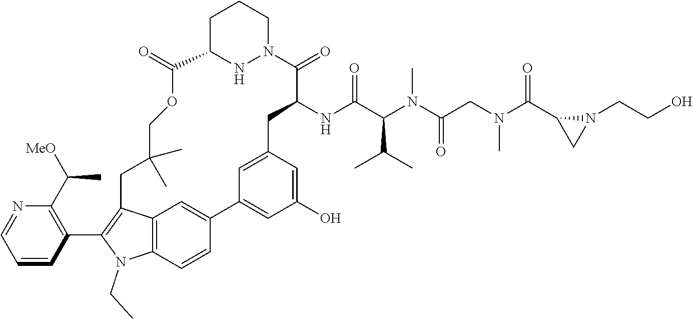 |
| C162 | 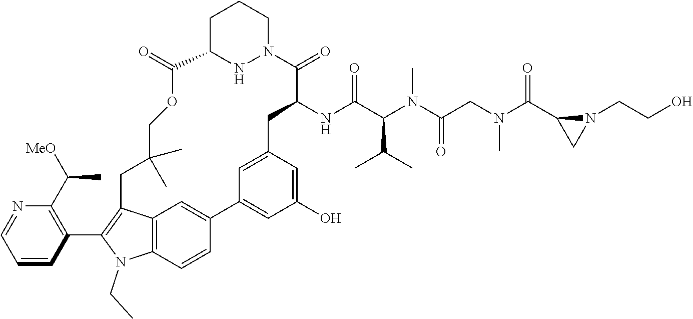 |
| C163 | 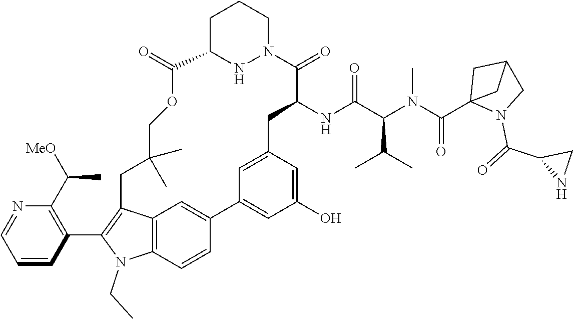 |
| C164 | 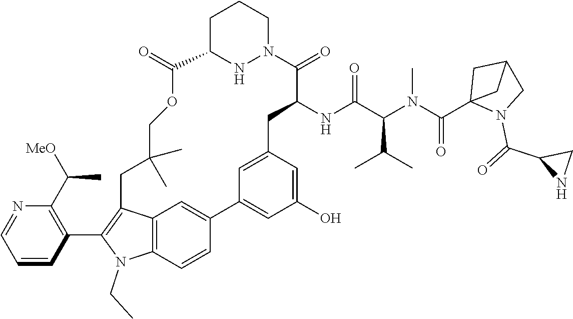 |
| C165 | 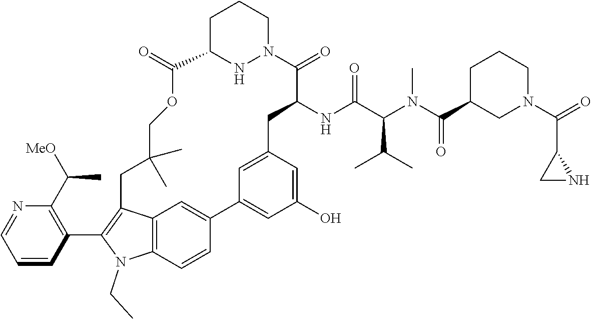 |
| C166 | 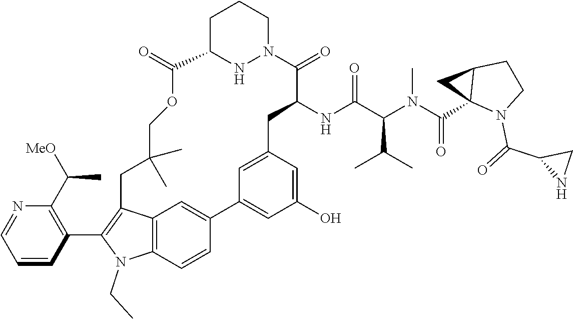 |
| C167 | 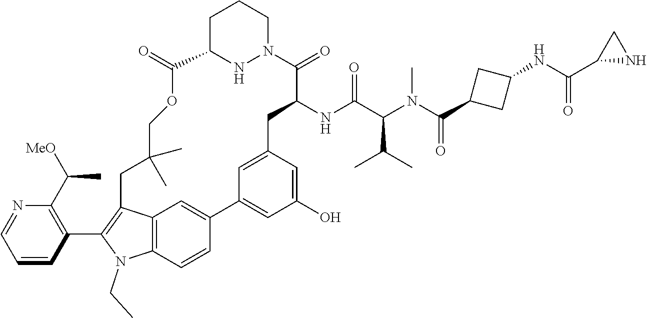 |
| C168 | 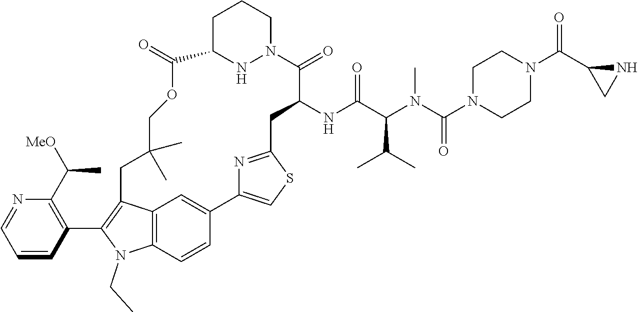 |
| C169 | 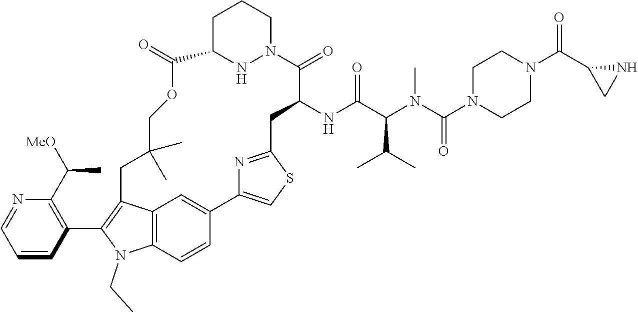 |
| C170 | 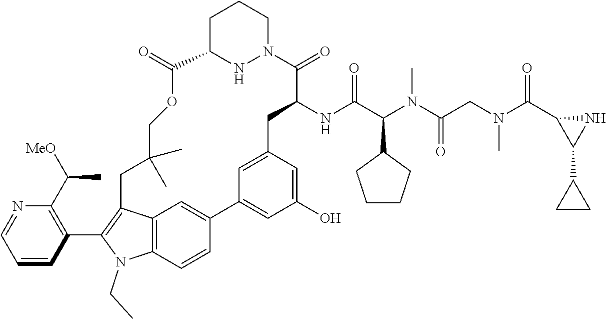 |
| C171 | 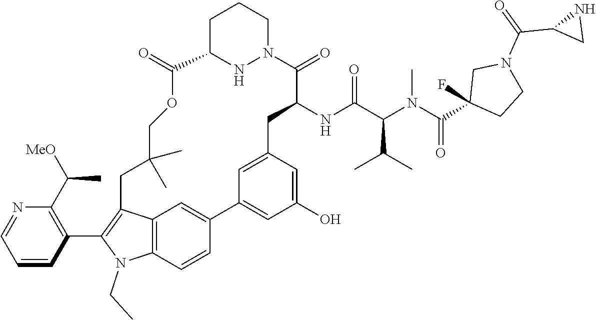 |
| C172 | 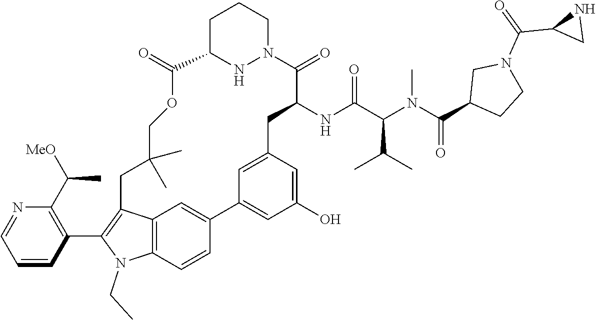 |
| C173 | 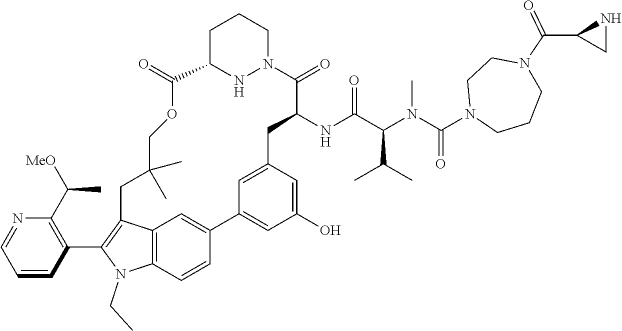 |
| C174 | 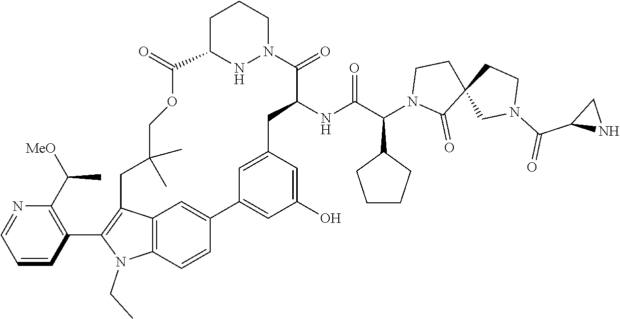 |
| C175 | 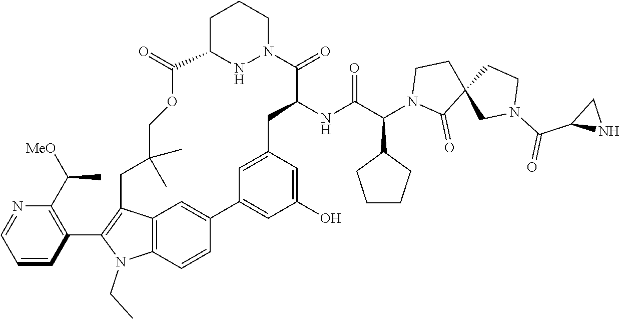 |
| C176 | 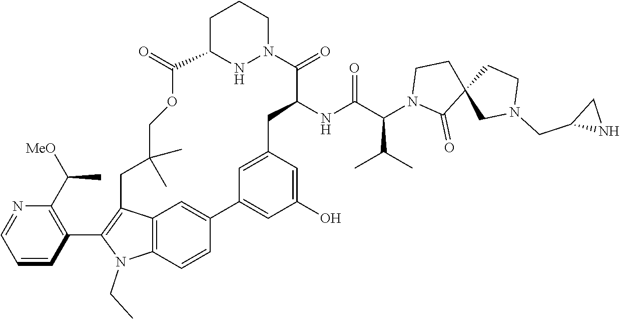 |
| C177 | 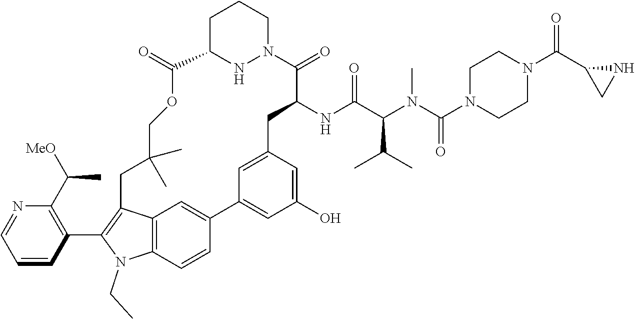 |
| C178 | 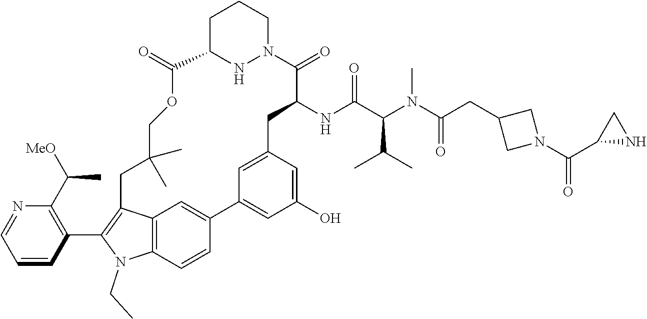 |
| C179 | 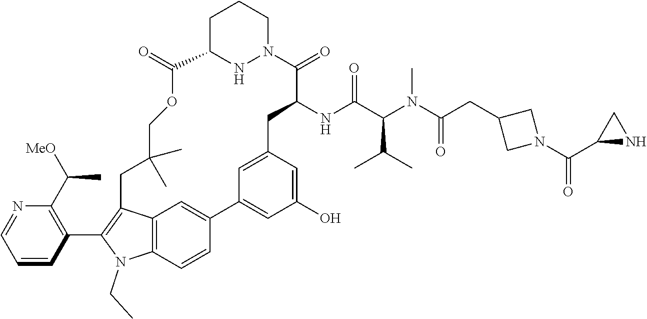 |
| C180 | 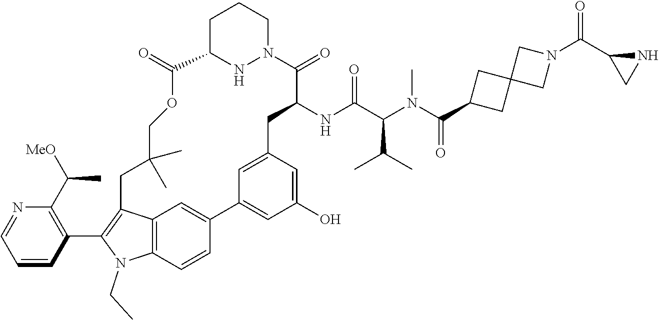 |
| C181 | 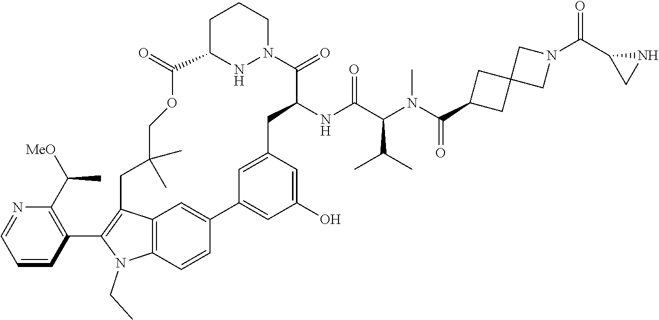 |
| C182 | 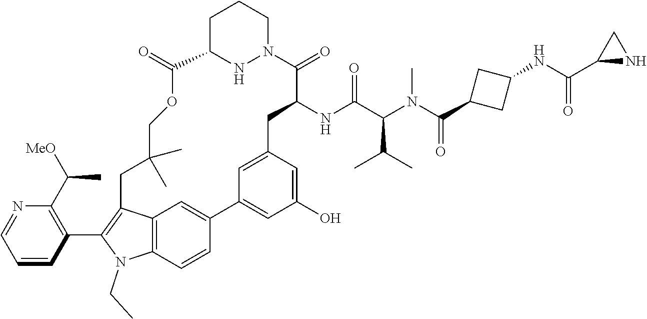 |
| C183 | 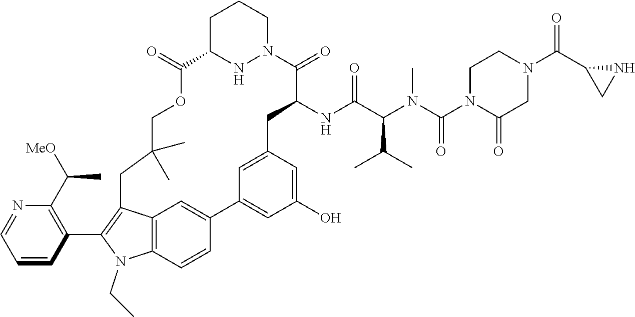 |
| C184 | 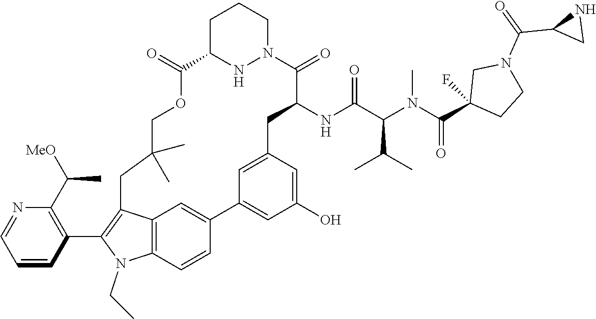 |
| C185 | 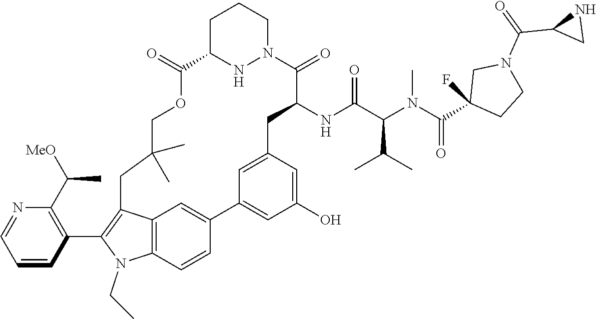 |
| C186 | 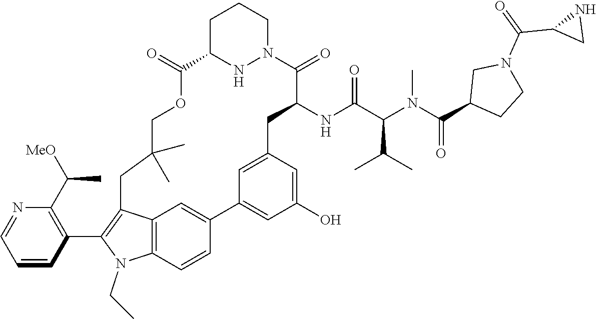 |
| C187 | 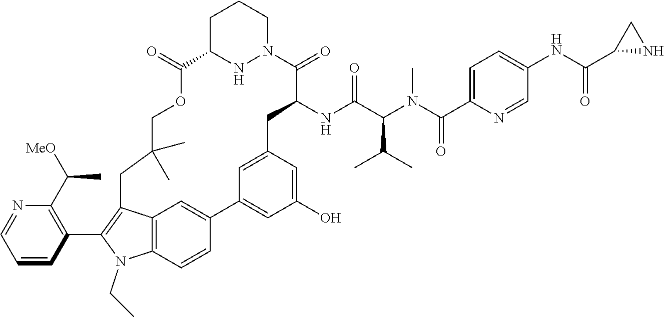 |
| C188 | 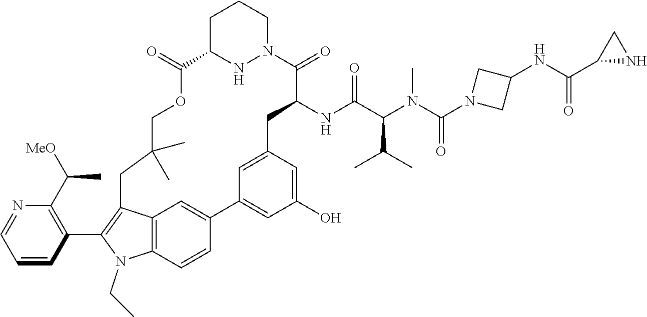 |
| C189 | 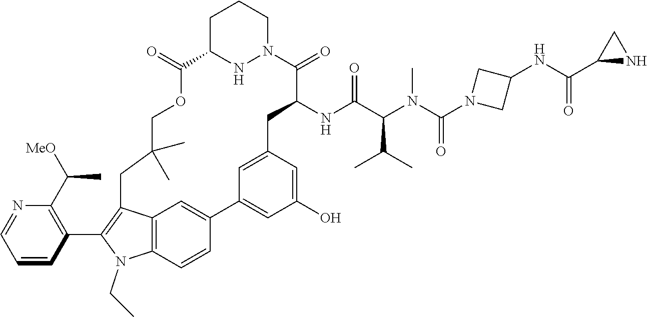 |
| C190 | 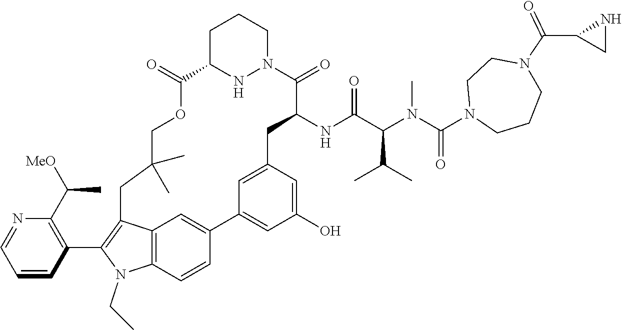 |
| C191 | 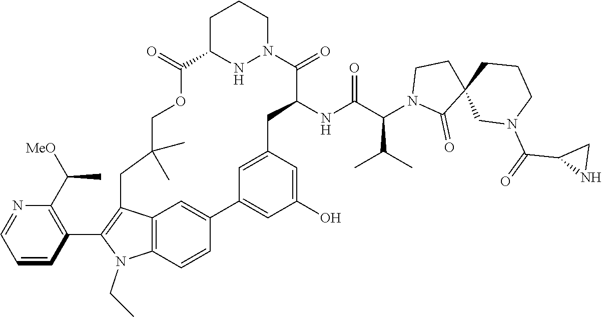 |
| C192 | 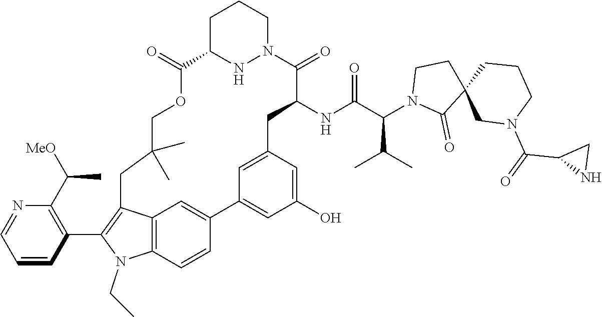 |
| C193 | 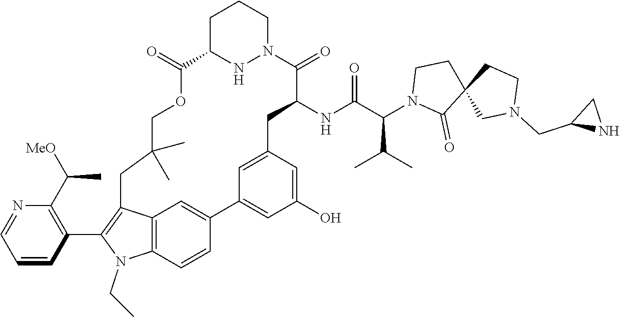 |
| C194 | 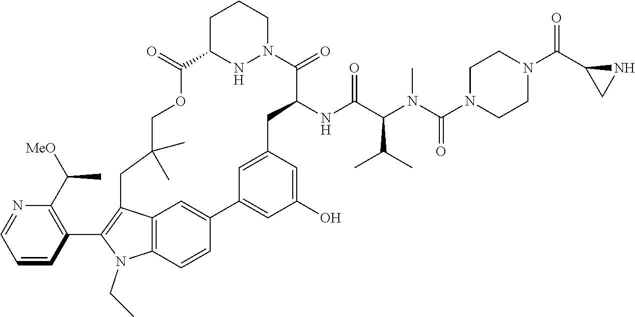 |
| C195 | 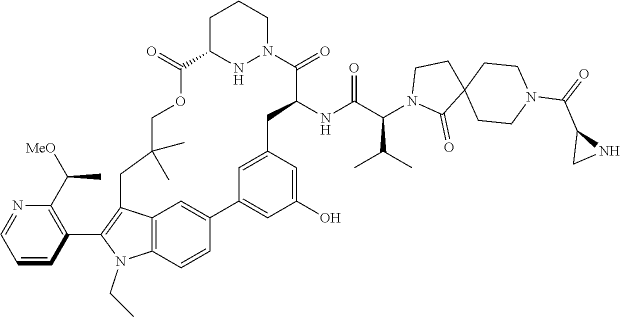 |
| C196 | 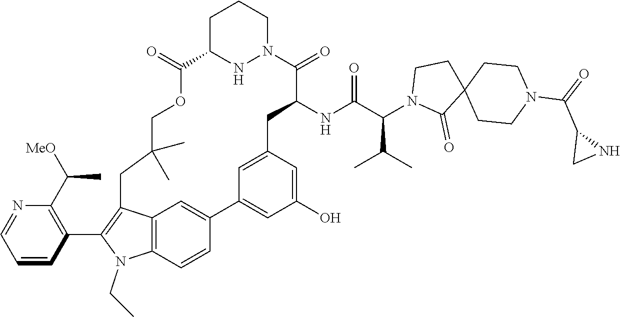 |
| C197 | 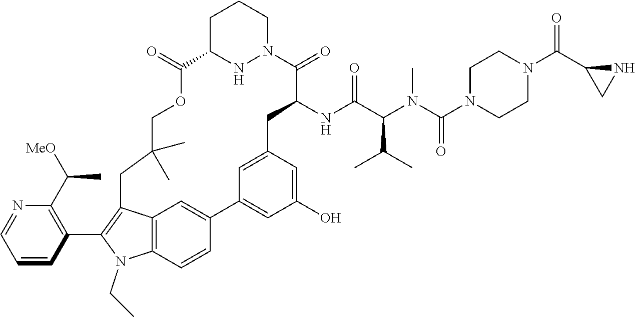 |
| C198 | 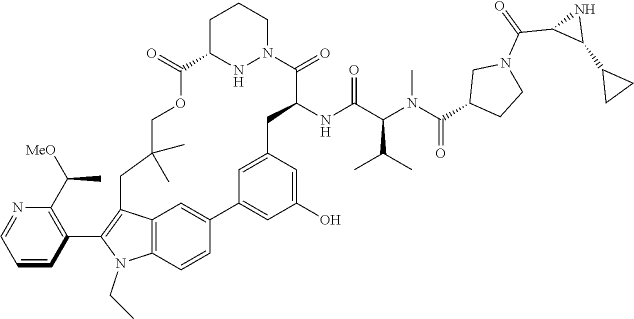 |
| C199 | 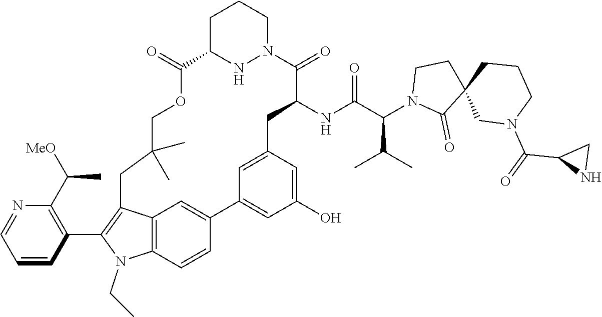 |
| C200 | 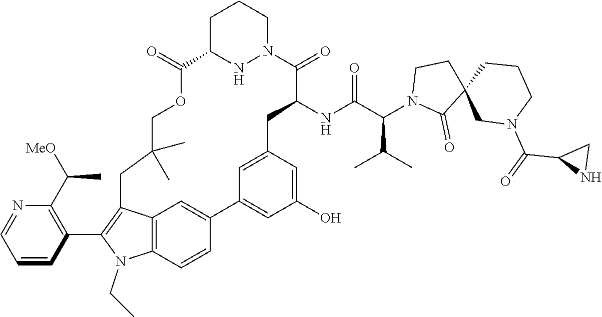 |
| C201 | 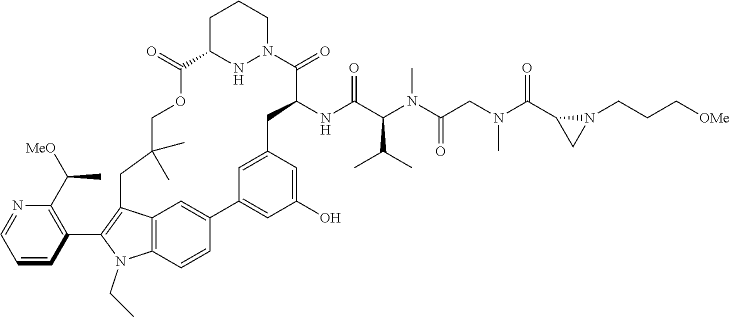 |
| C202 | 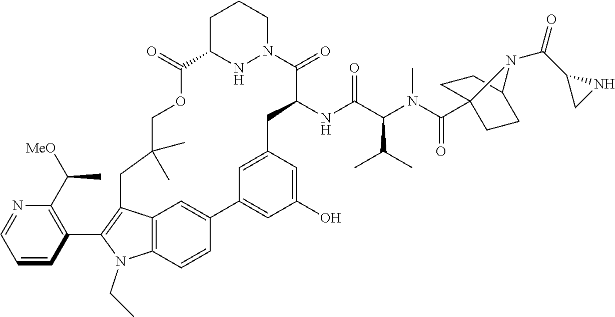 |
| C203 | 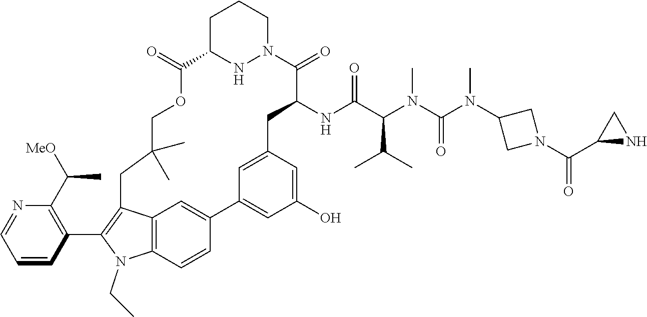 |
| C204 | 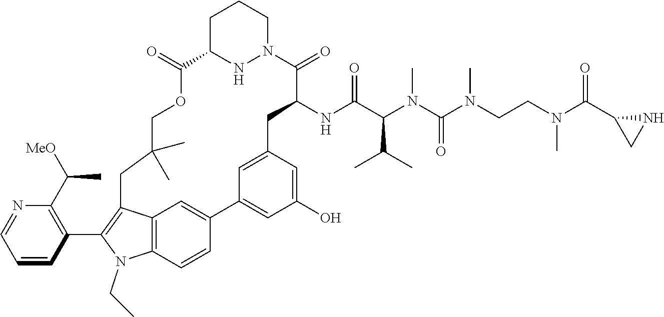 |
| C205 | 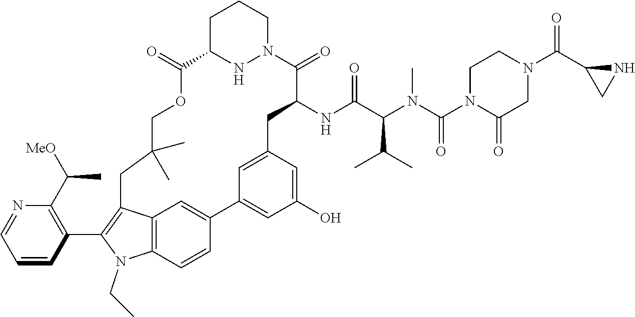 |
| C206 | 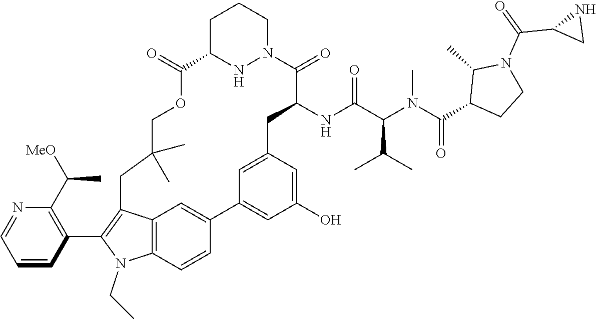 |
| C207 | 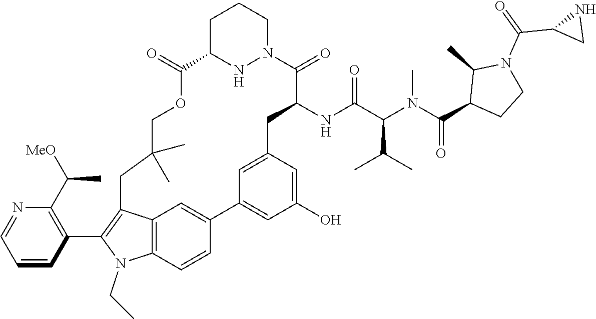 |
| C208 | 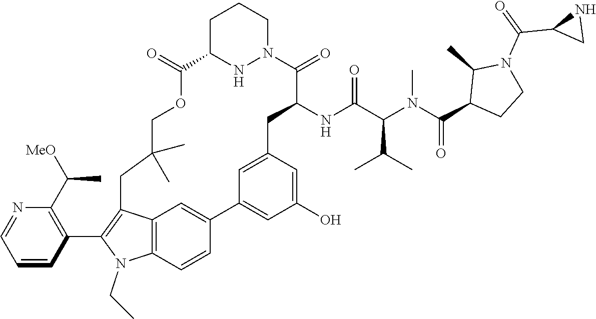 |
| C209 | 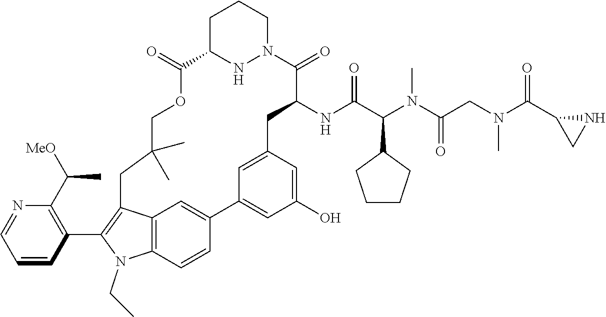 |
| C210 | 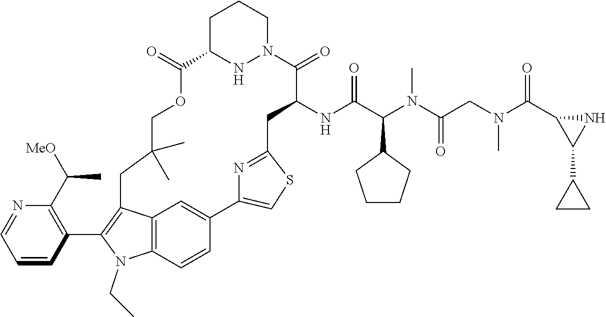 |
| C211 | 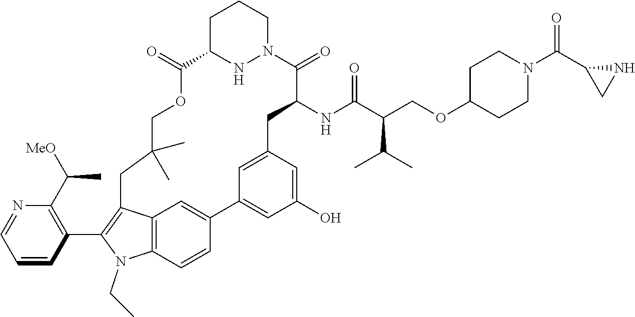 |
| C212 | 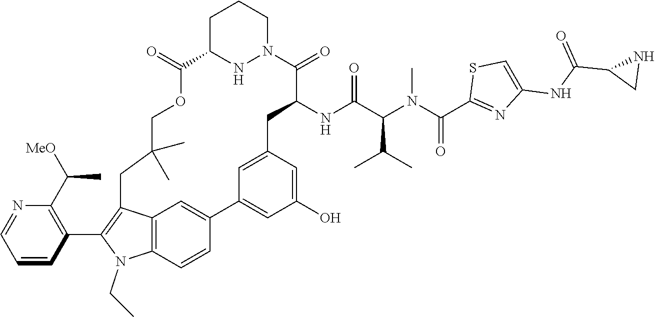 |
| C213 | 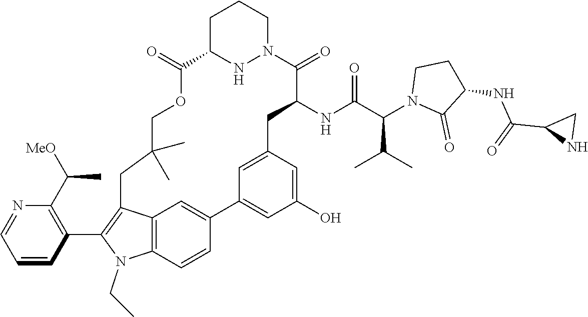 |
| C214 | 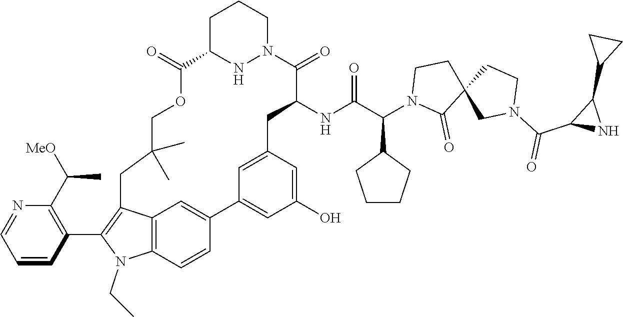 |
| C215 | 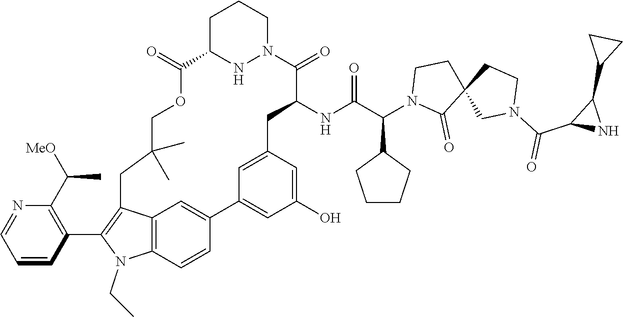 |
| C216 | 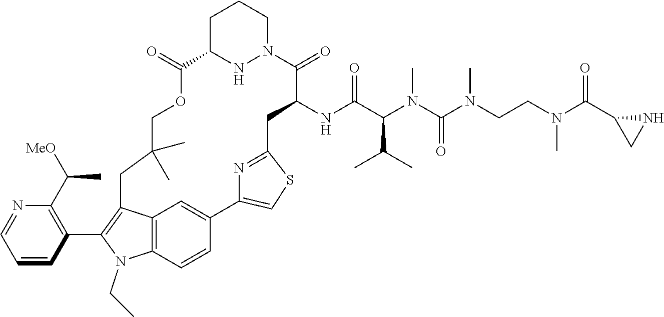 |
| C217 | 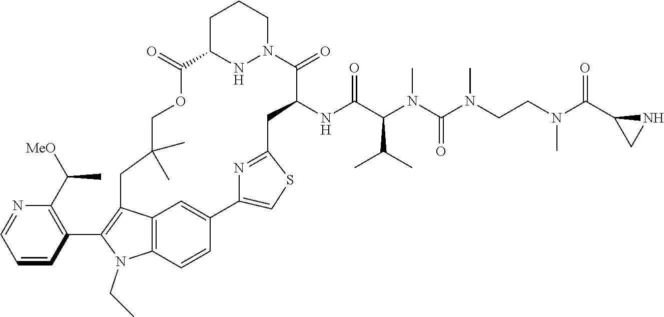 |
| C218 | 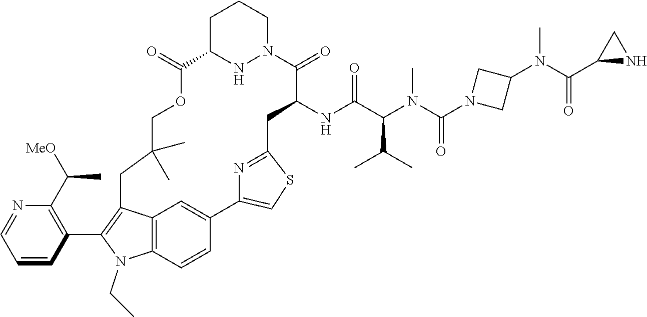 |
| C219 | 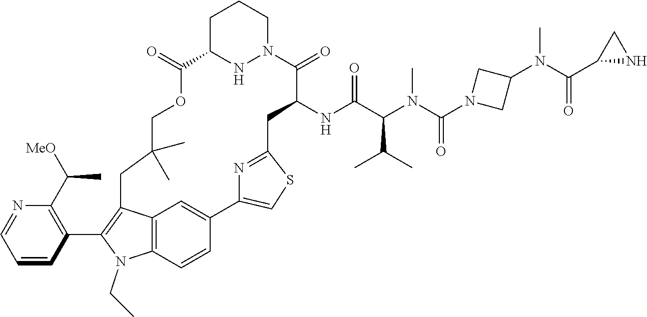 |
| C220 | 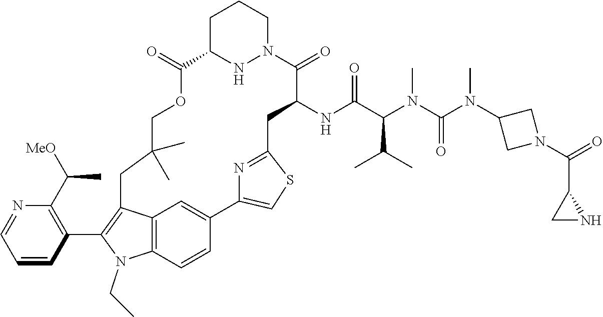 |
| C221 | 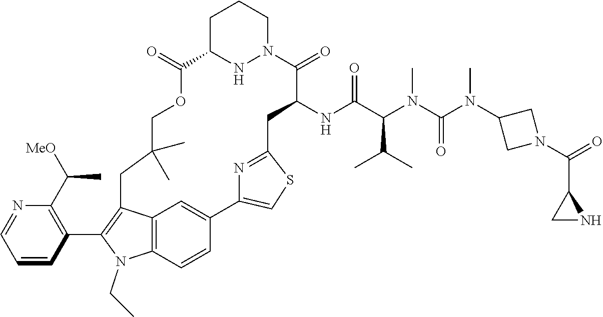 |
| C222 | 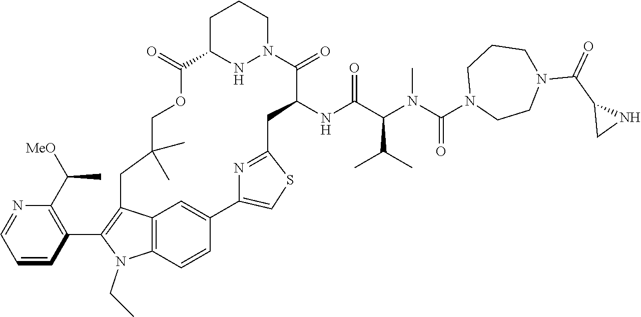 |
| C223 | 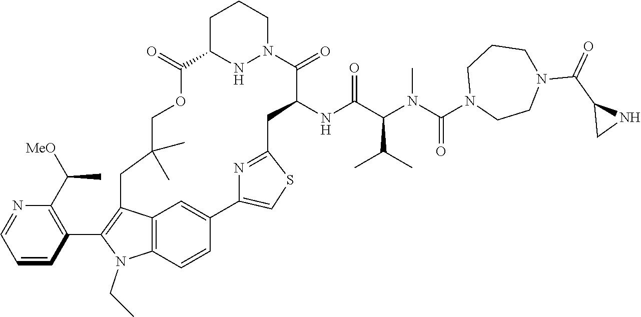 |
| C224 | 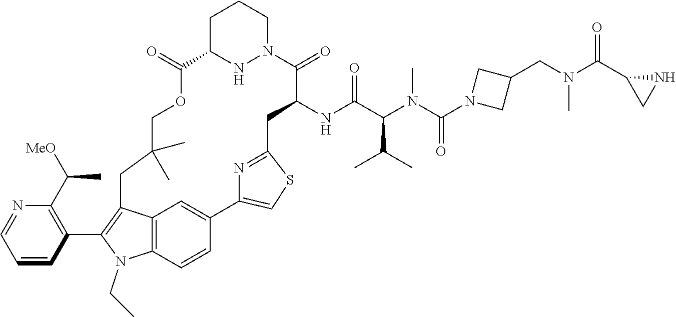 |
| C225 | 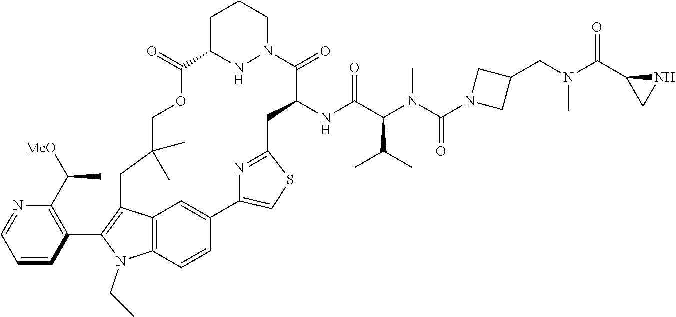 |
| C226 | 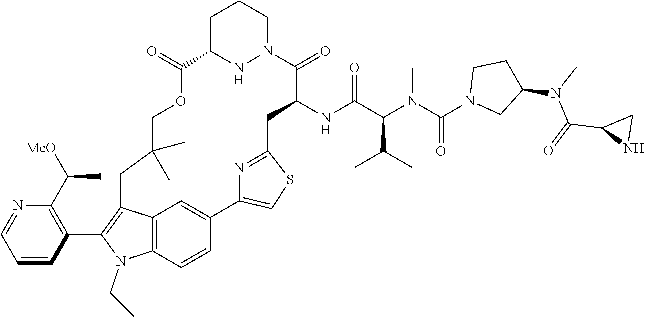 |
| C227 | 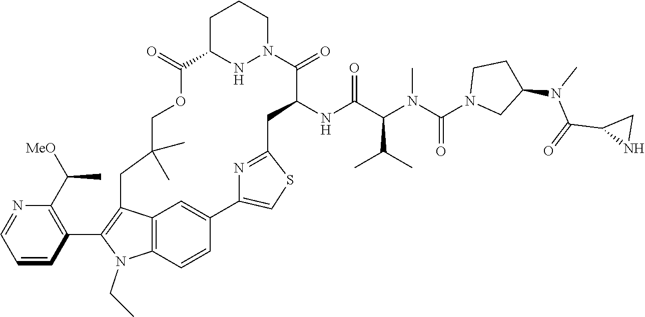 |
| C228 | 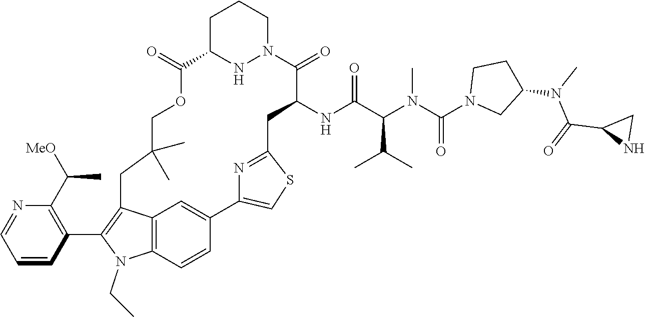 |
| C229 | 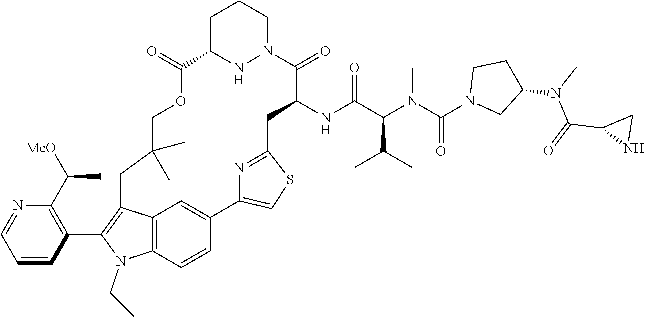 |
| C230 | 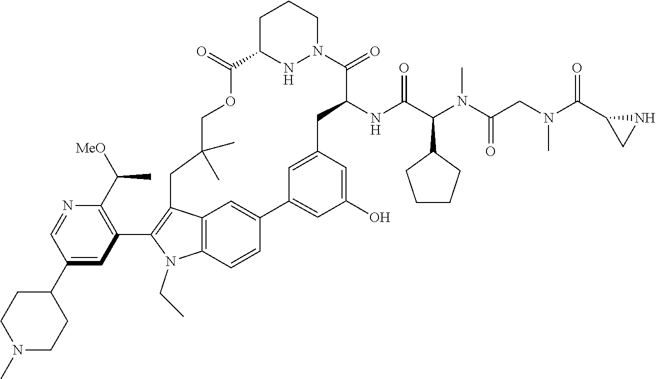 |
| C231 | 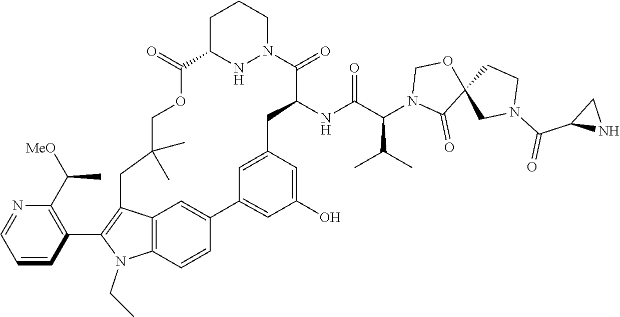 |
| C232 | 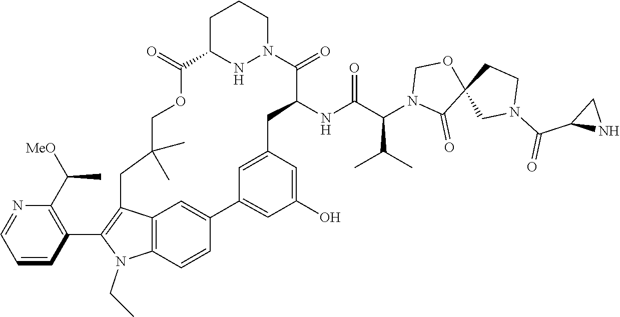 |
| C233 | 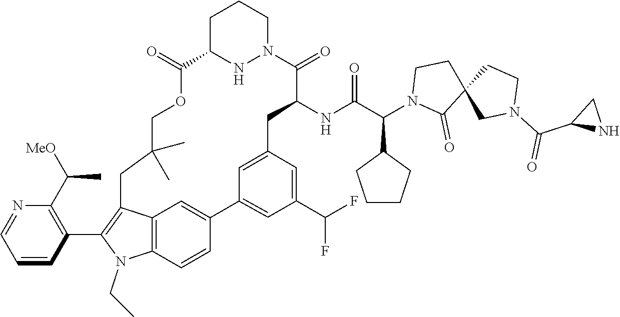 |
| C234 | 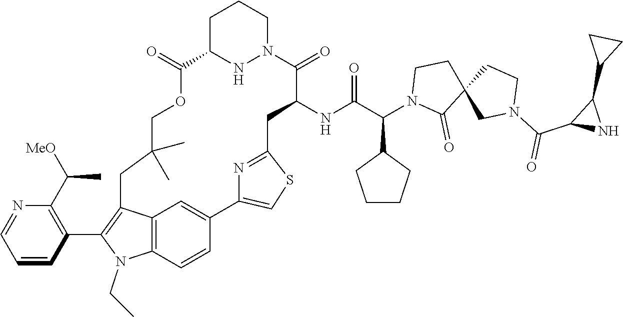 |
| C235 | 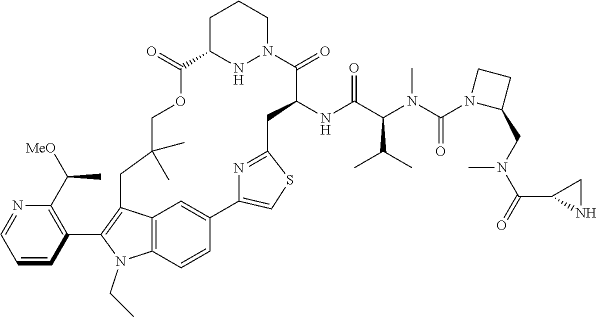 |
| C236 | 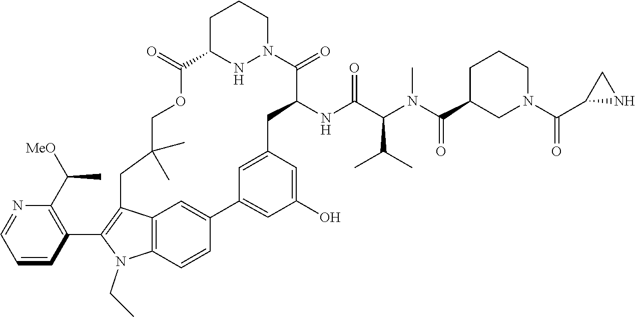 |
| C237 | 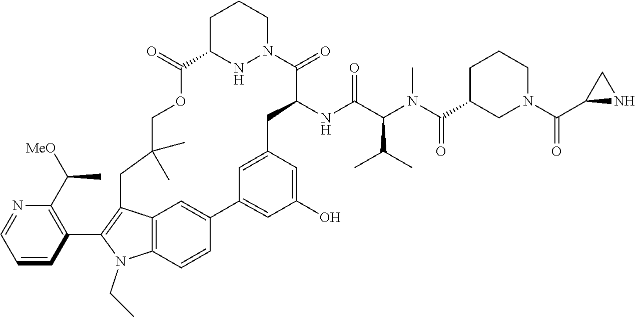 |
| C238 | 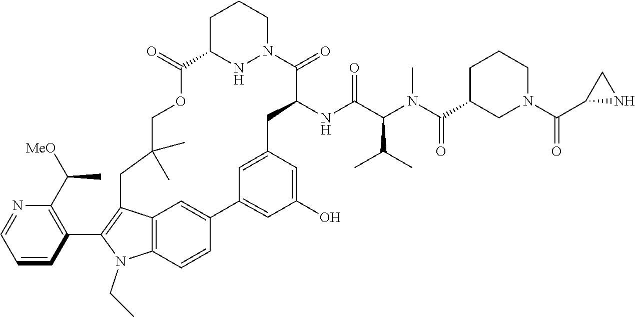 |
| C239 | 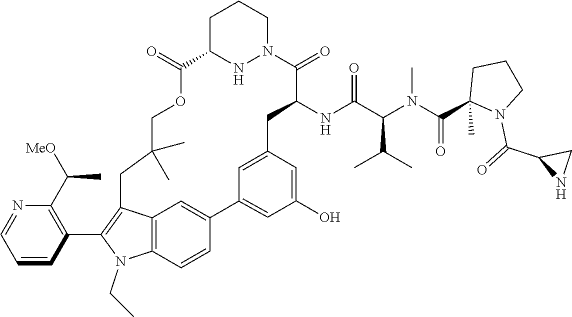 |
| C240 | 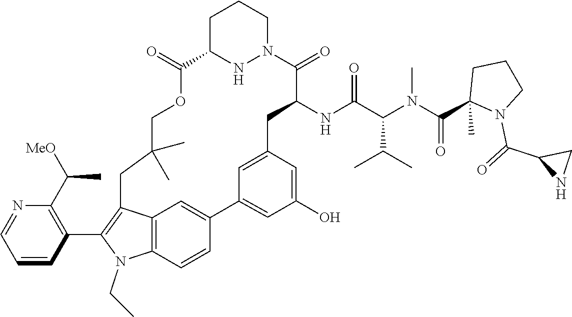 |
| C241 | 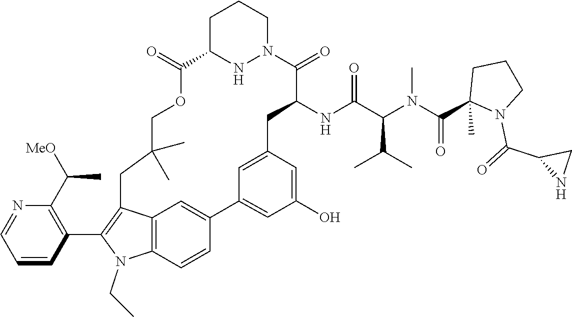 |
| C242 | 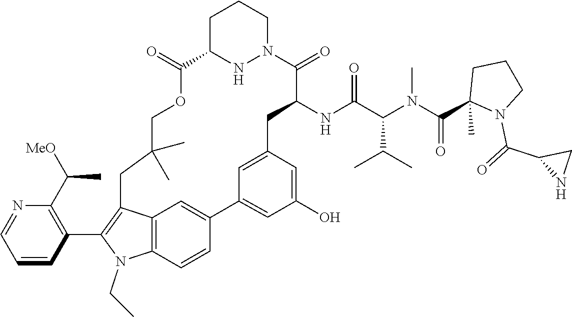 |
| C243 | 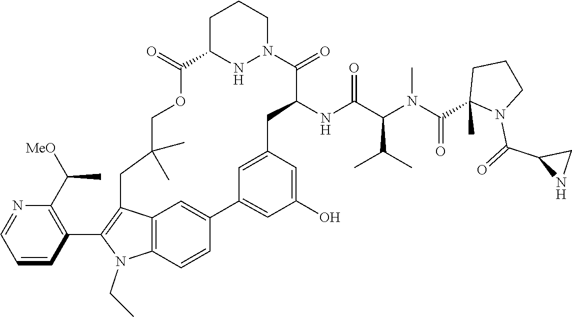 |
| C244 | 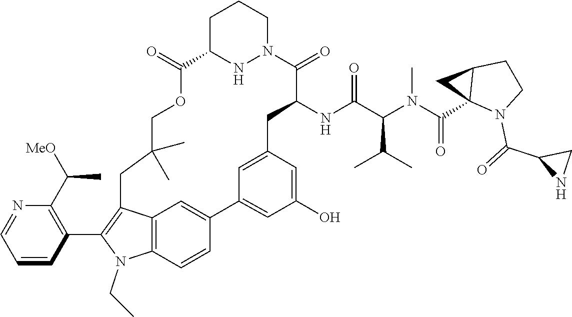 |
| C245 | 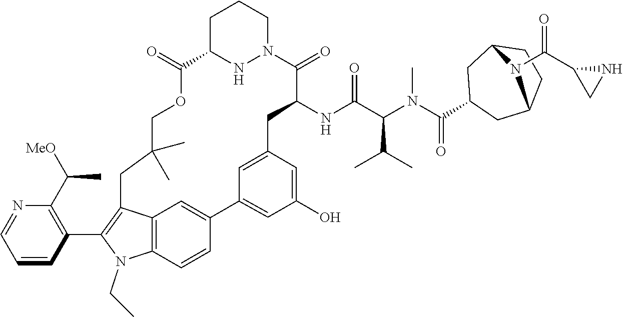 |
| C246 | 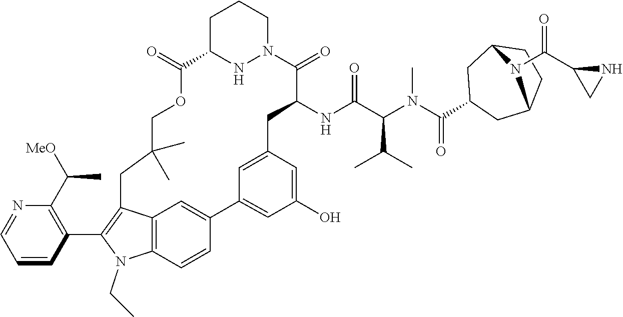 |
| C247 | 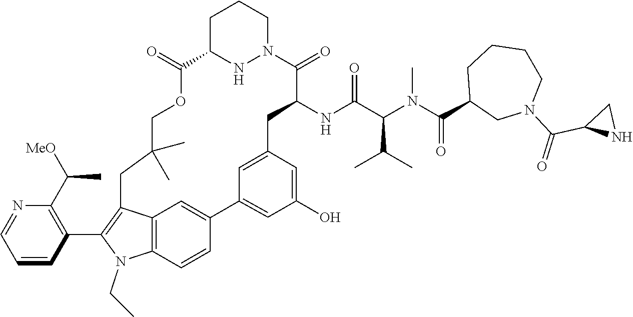 |
| C248 | 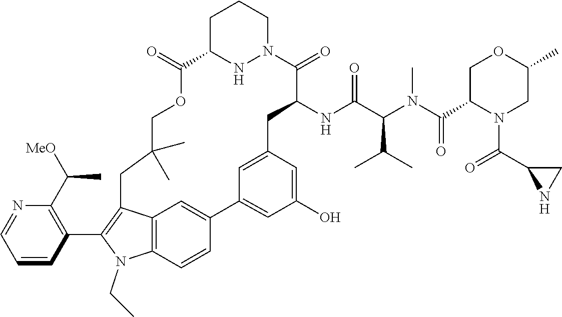 |
| C249 | 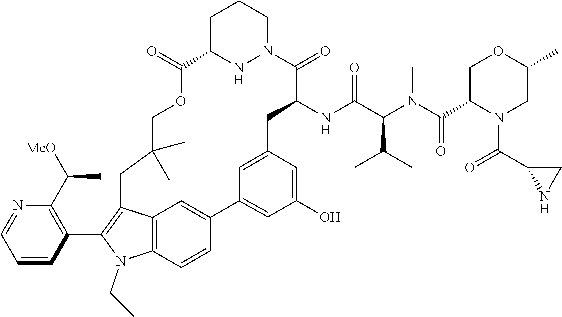 |
| C250 | 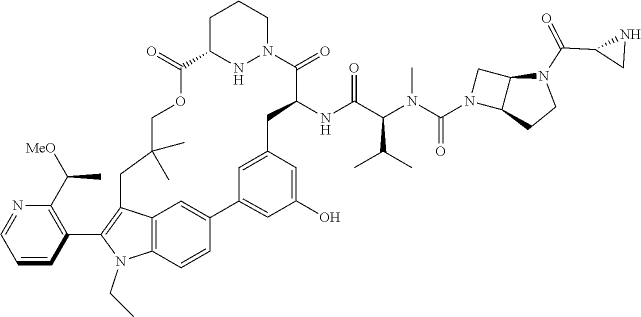 |
| C251 | 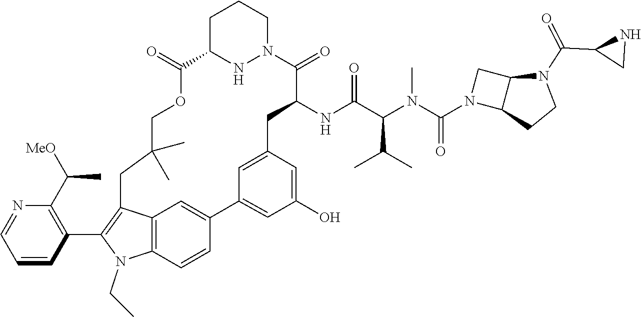 |
| C252 | 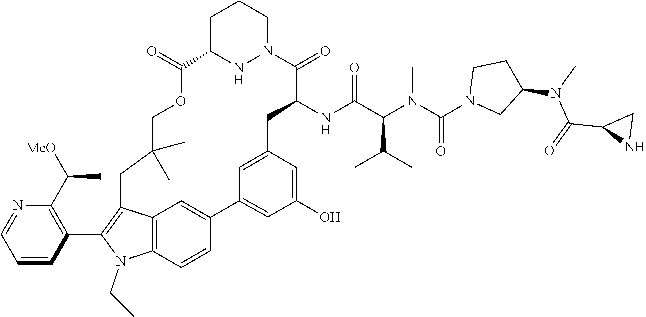 |
| C253 | 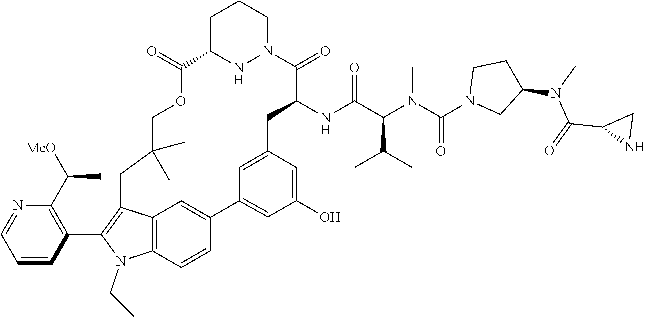 |
| C254 | 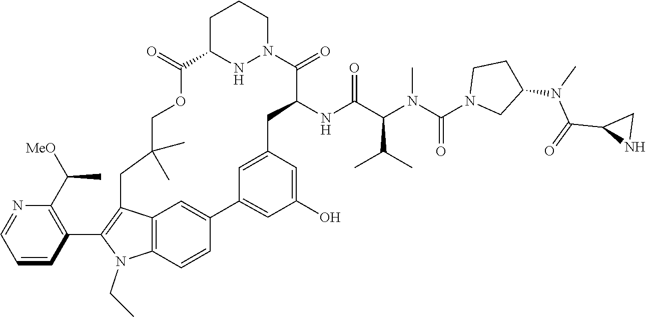 |
| C255 | 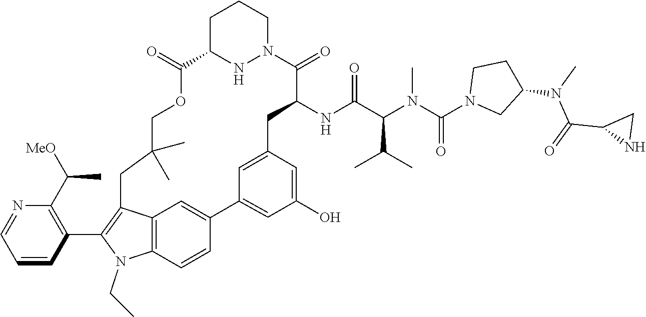 |
| C256 | 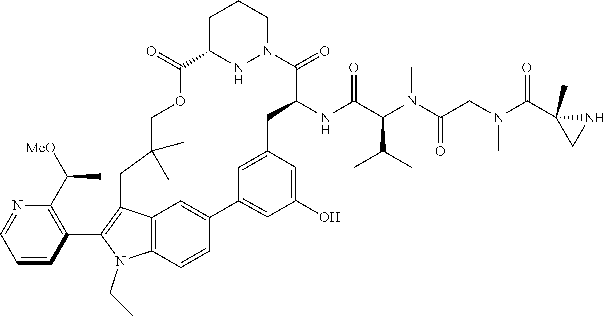 |
| C257 | 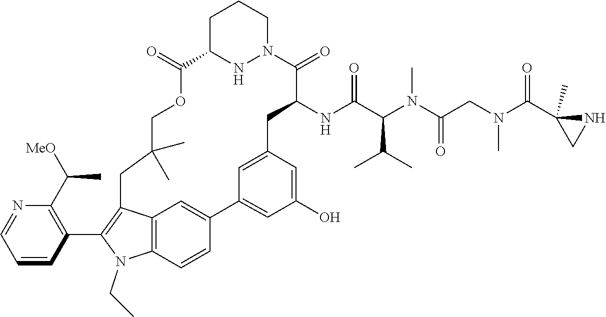 |
| C258* | 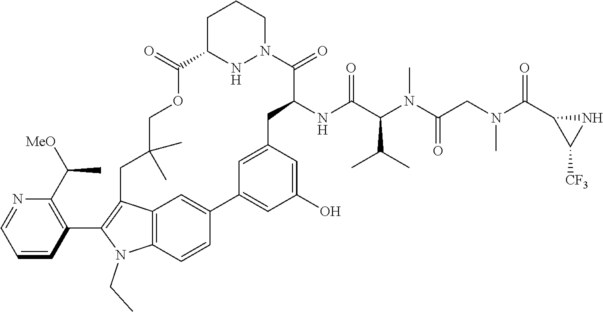 |
| C259* | 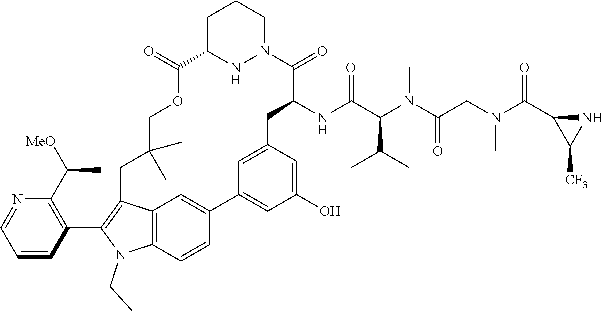 |
| C260* | 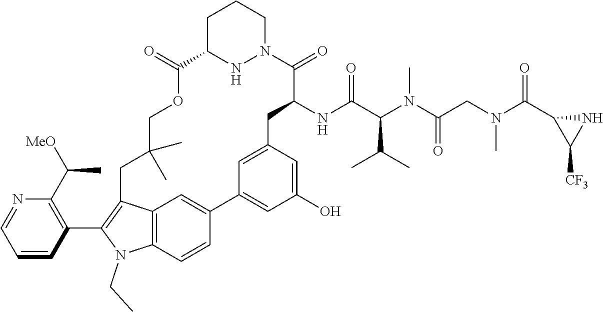 |
| C261* | 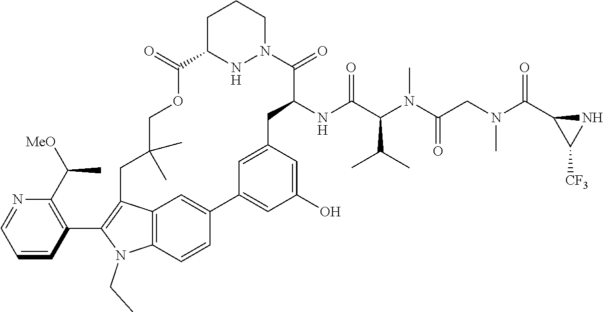 |
| C262 | 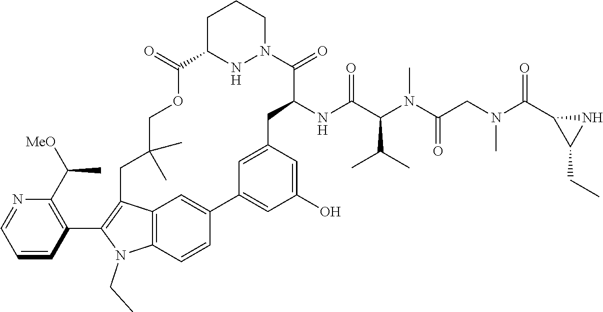 |
| C263 | 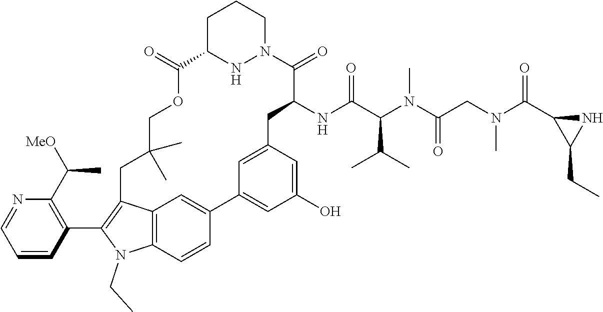 |
| C264 | 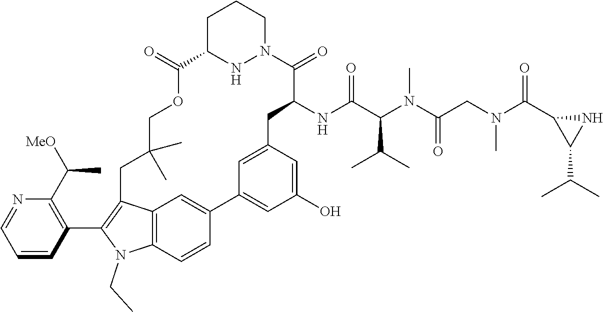 |
| C265 | 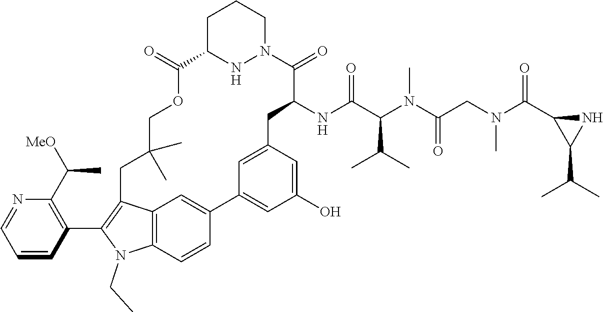 |
| C266 | 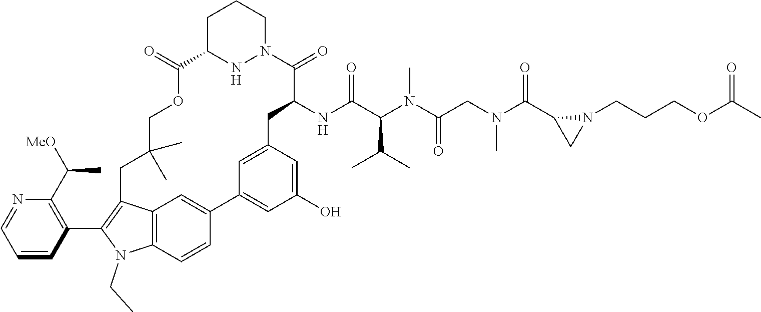 |
| C267 | 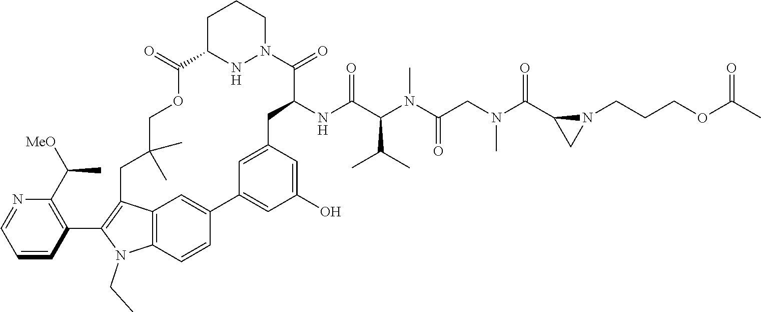 |
| C268 | 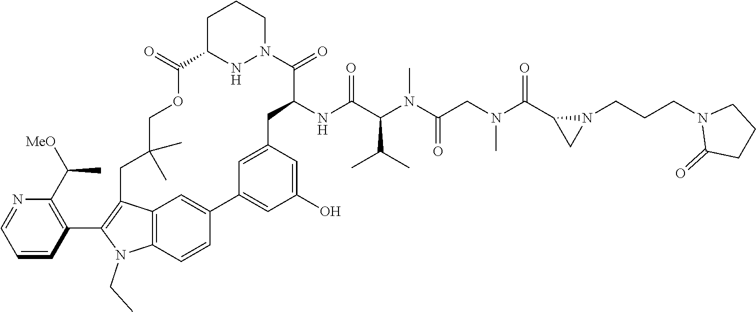 |
| C269 | 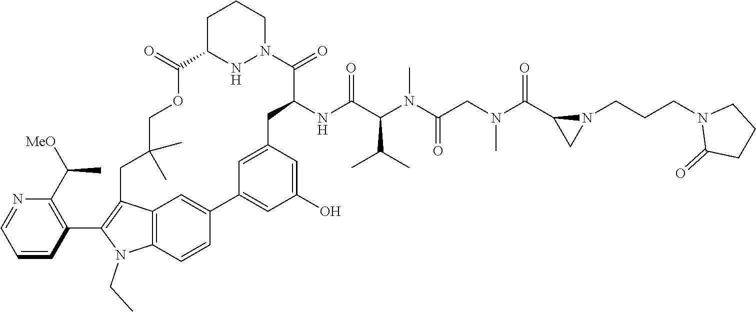 |
| C270 | 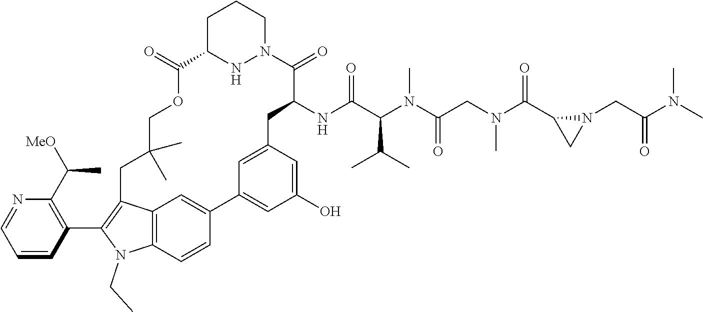 |
| C271 | 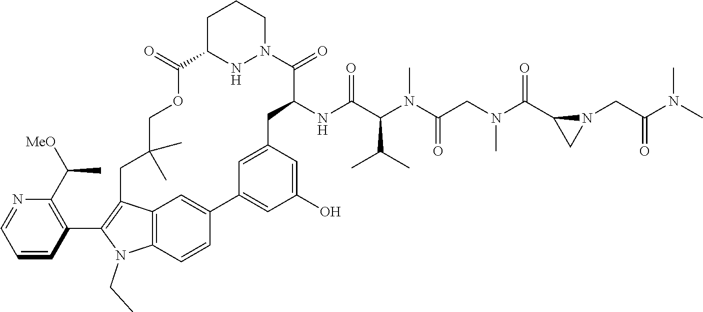 |
| C272 | 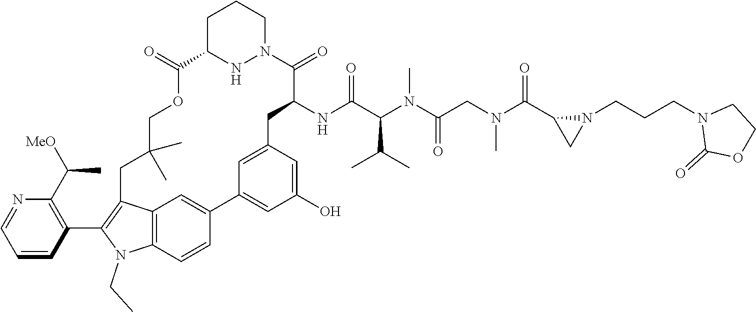 |
| C273 | 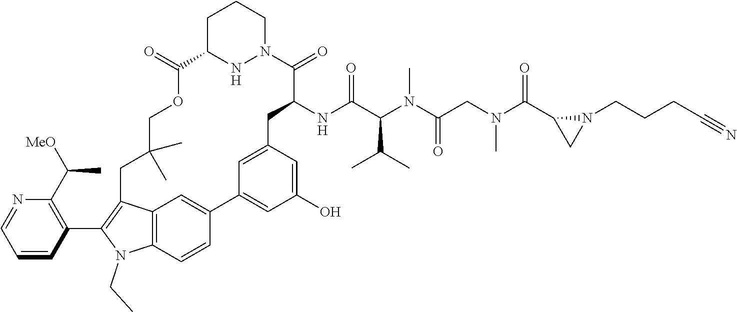 |
| C274 | 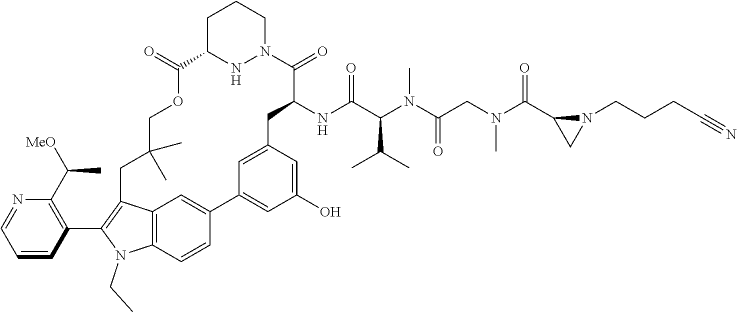 |
| C275 | 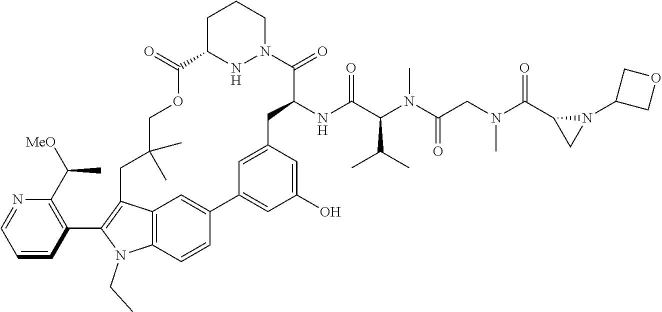 |
| C276 | 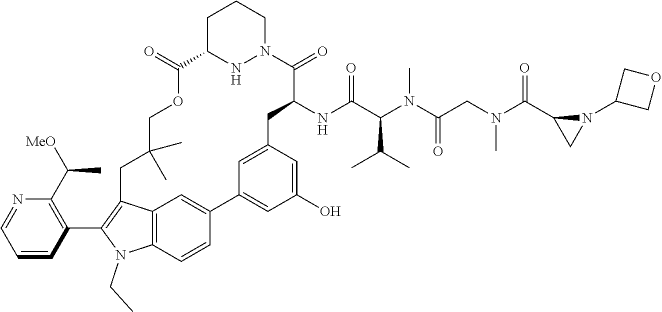 |
| C277 | 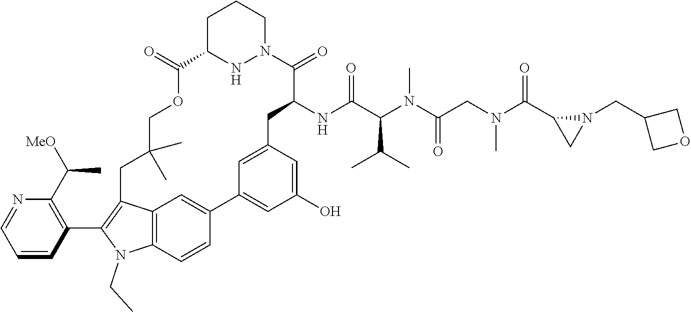 |
| C278 | 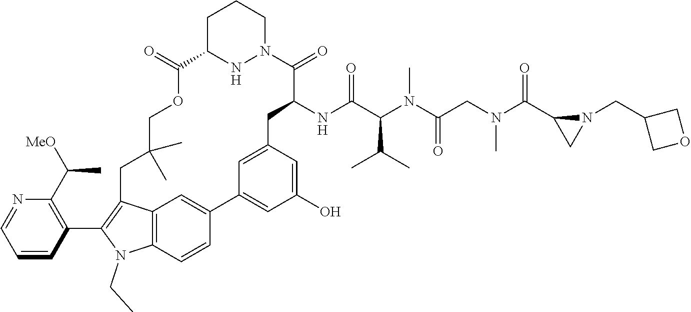 |
| C279 | 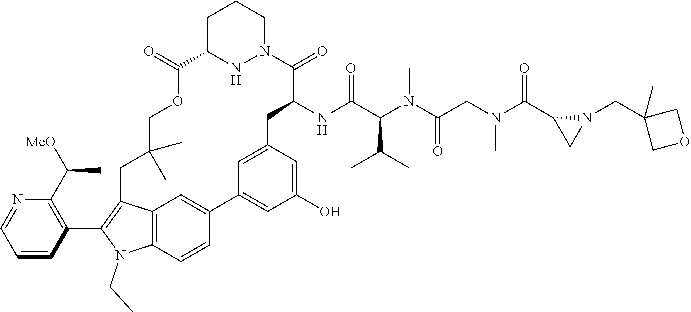 |
| C280 | 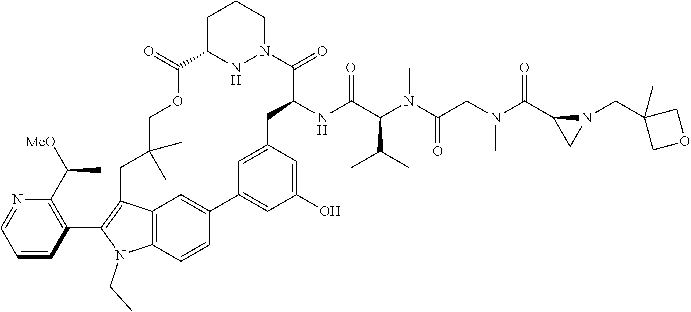 |
| C281* | 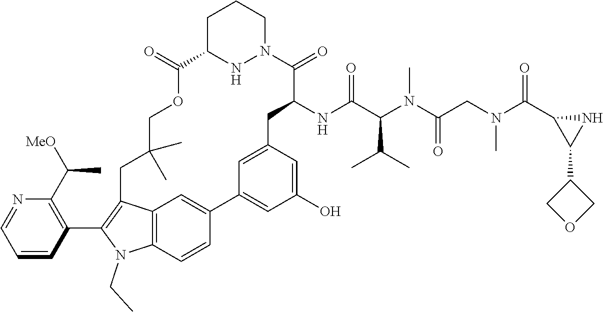 |
| C282* | 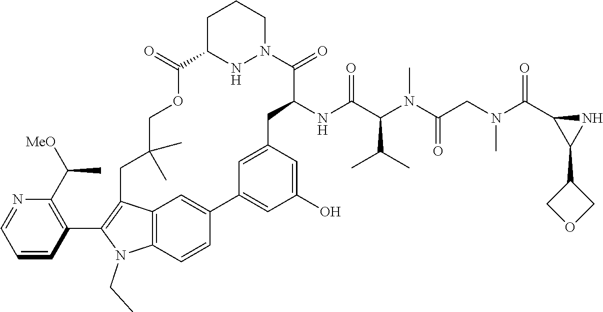 |
| C283* | 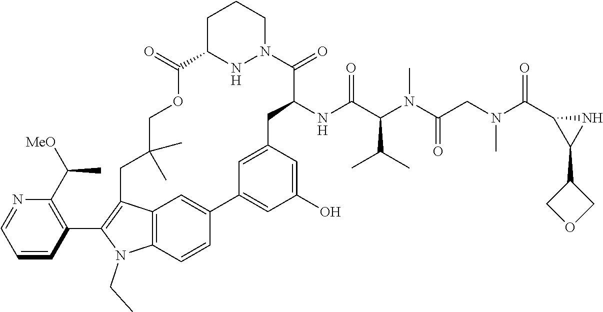 |
| C284* | 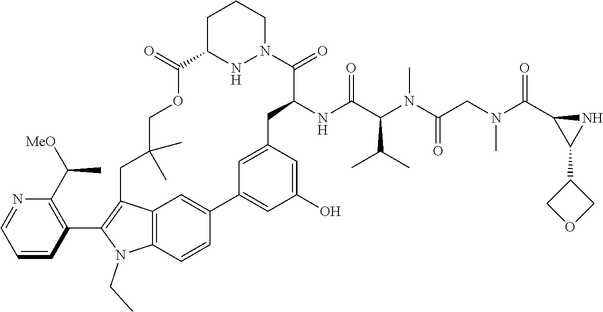 |
| C285 | 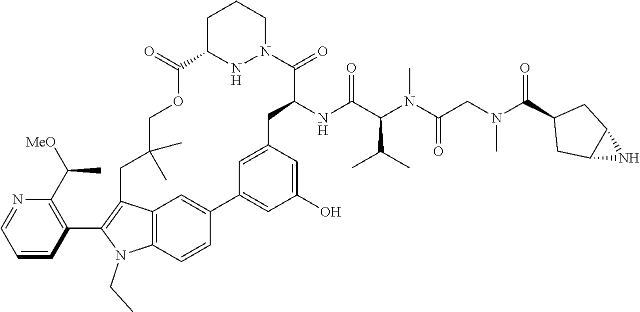 |
| C286 | 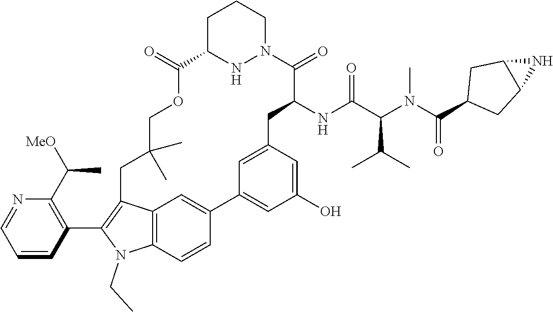 |
| C287 | 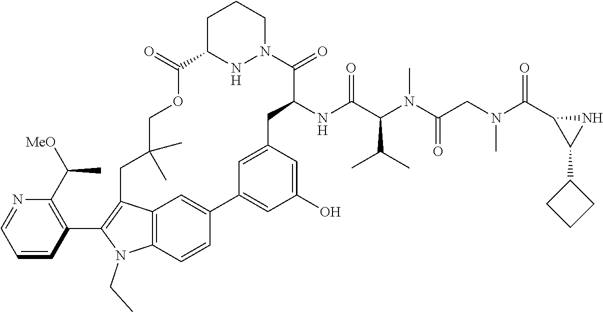 |
| C288 | 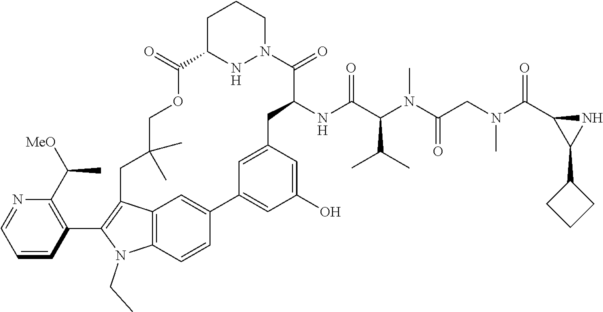 |
| C289 | 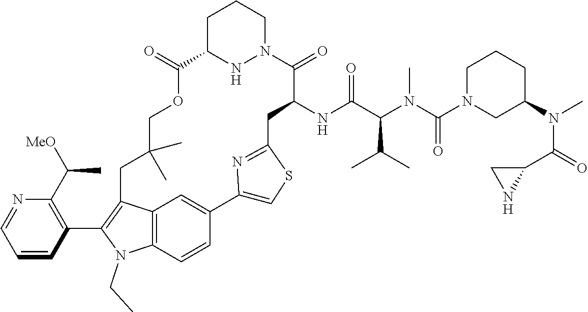 |
| C290 | 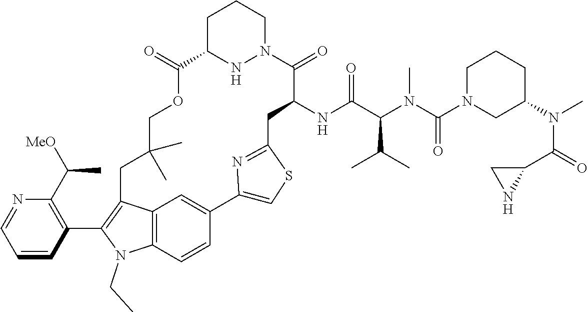 |
| C291 | 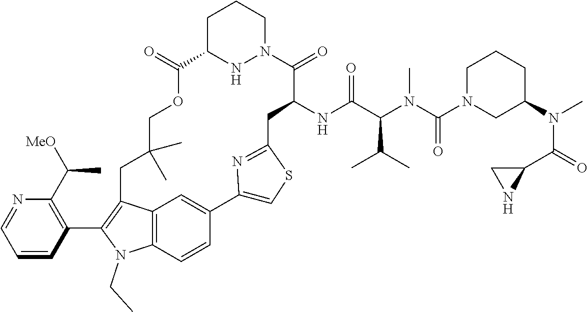 |
| C292 | 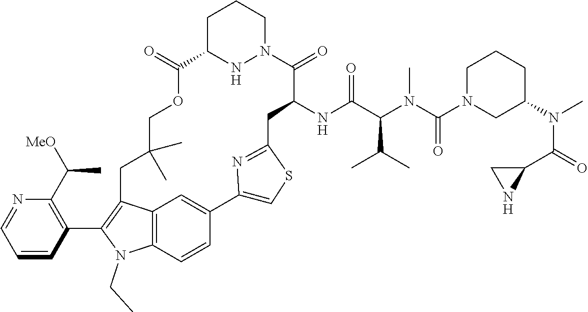 |
| C293 | 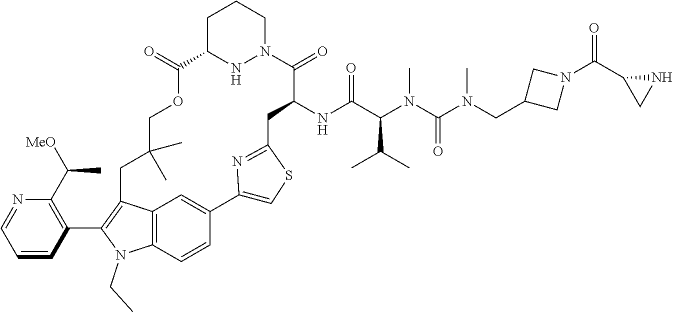 |
| C294 | 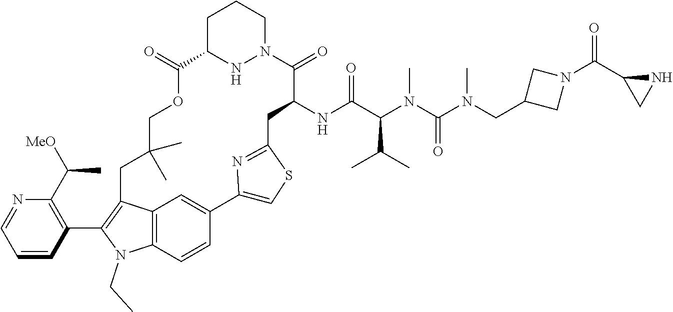 |
| C295 | 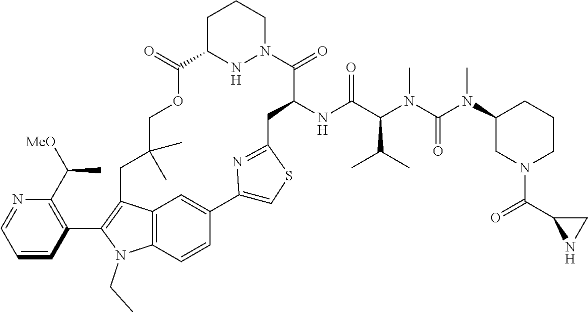 |
| C296 | 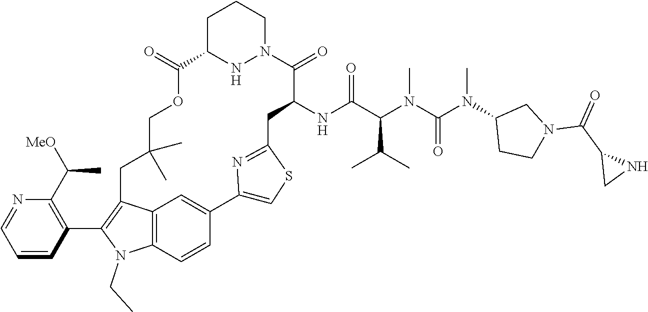 |
| C297 | 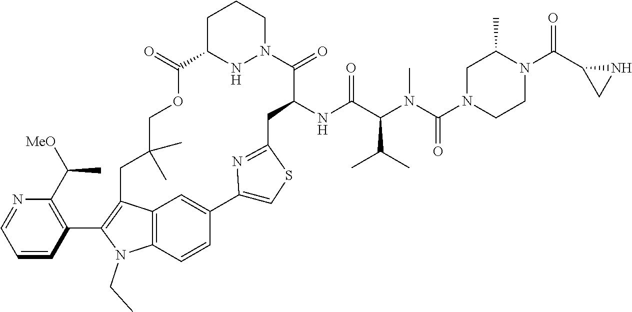 |
| C298 | 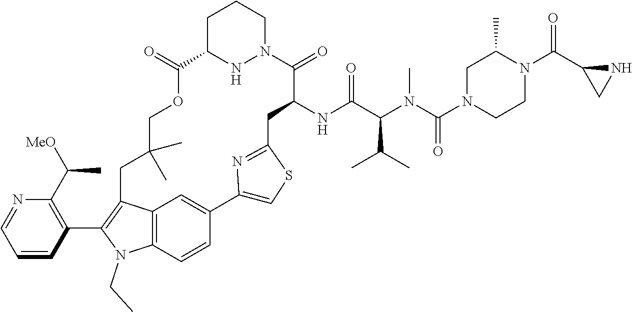 |
| C299 | 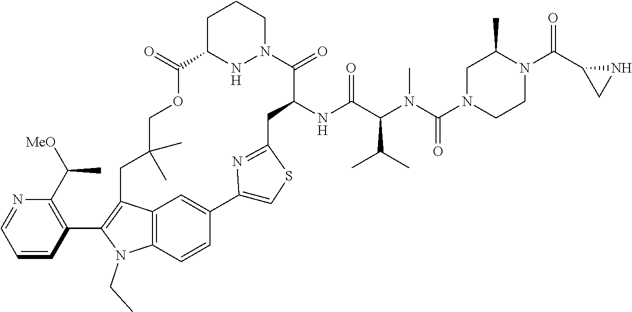 |
| C300 | 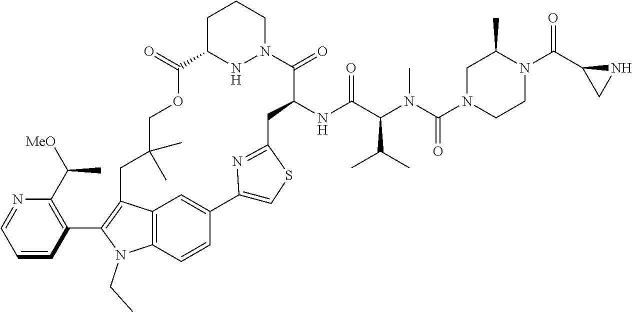 |
| C301 | 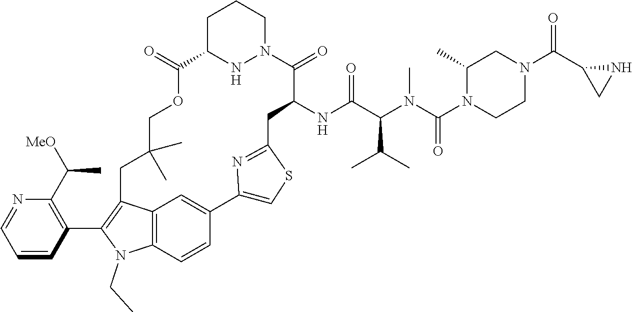 |
| C302 | 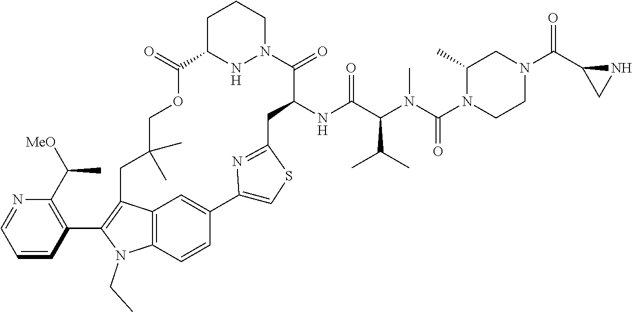 |
| C303 | 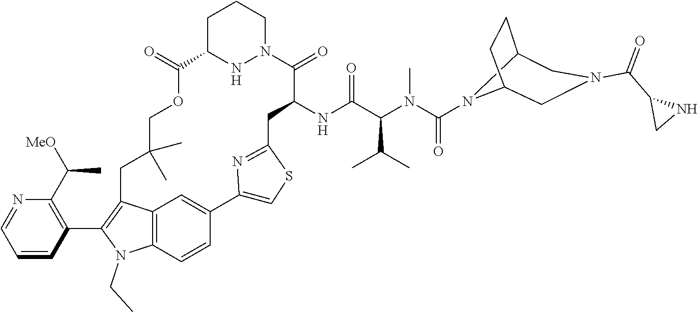 |
| C304 | 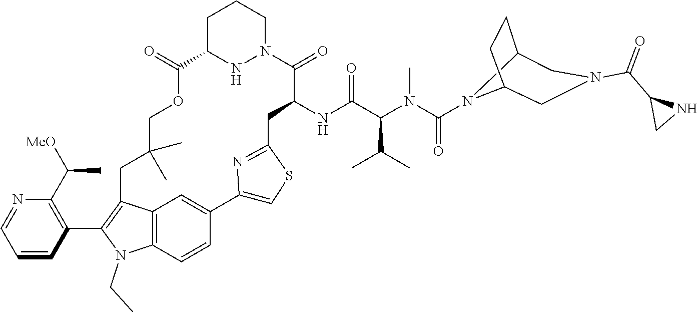 |
| C305 | 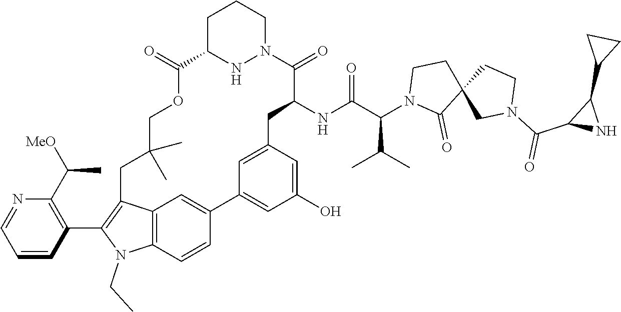 |
| C306 | 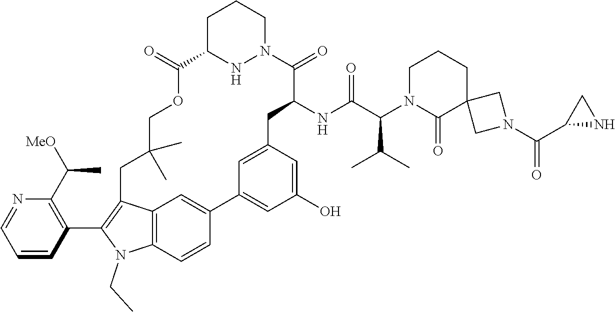 |
| C307 | 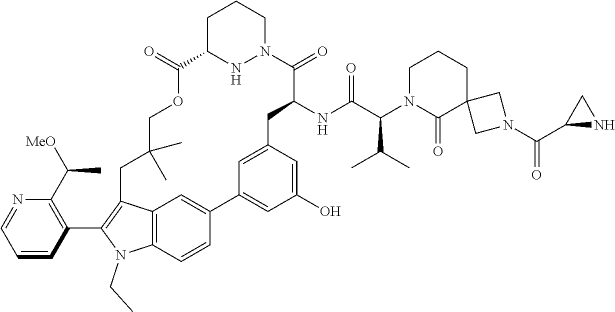 |
| C308* | 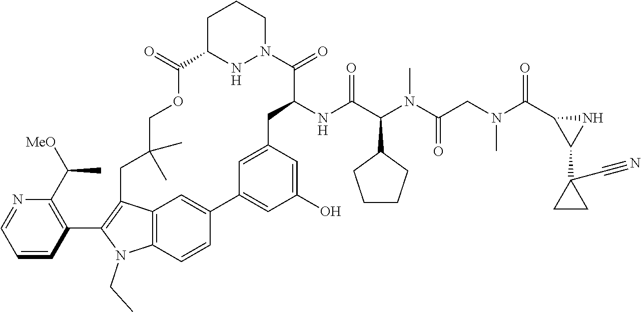 |
| C309 | 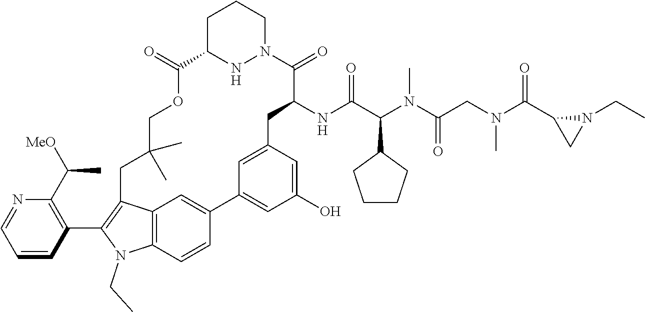 |
| C310 | 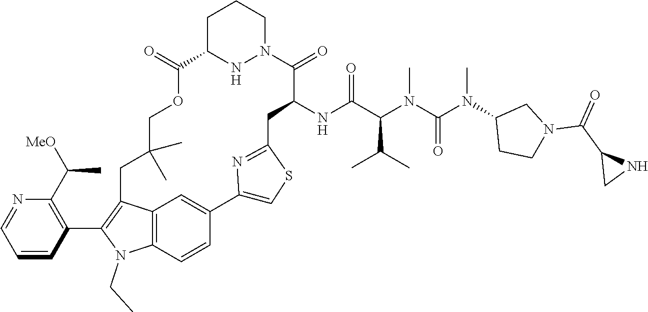 |
| C311 | 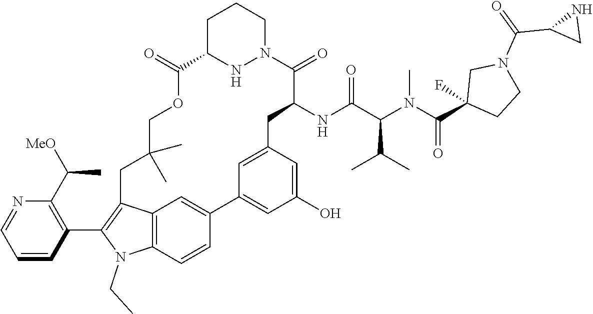 |
| C312 | 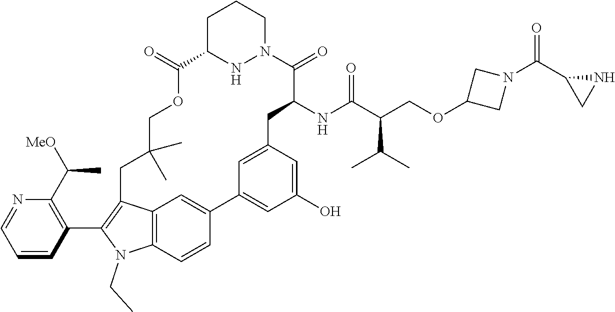 |
| C313 | 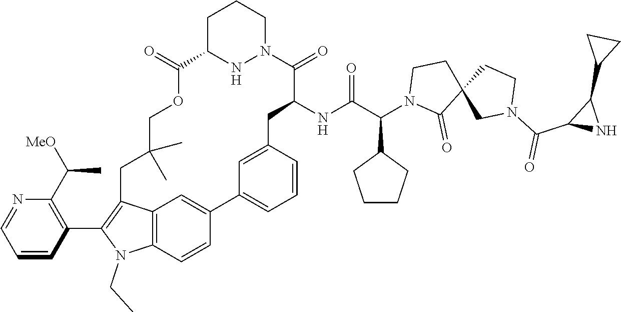 |
| C314* | 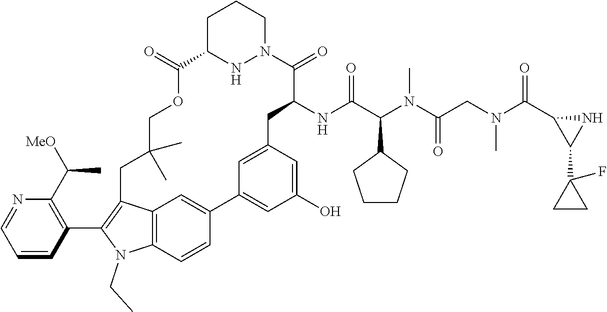 |
| C315* | 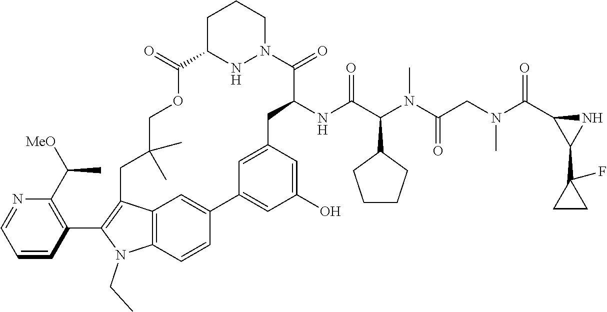 |
| C316* | 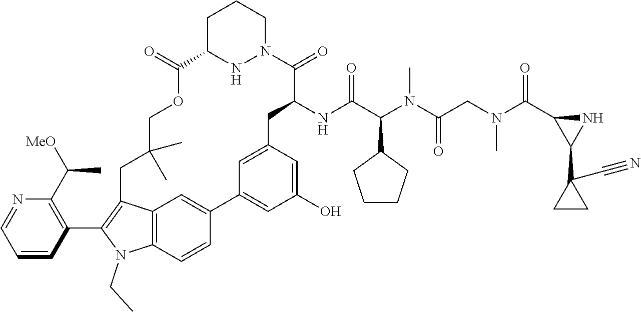 |
| C317 | 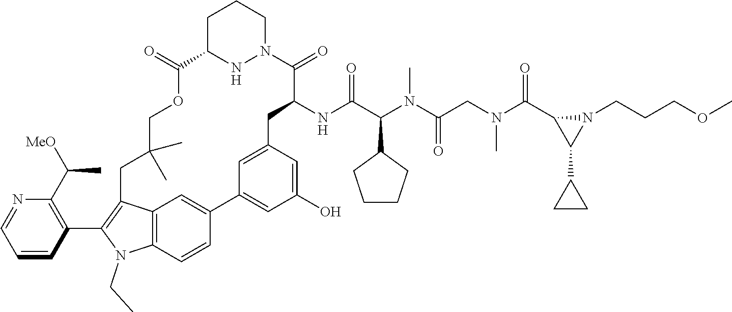 |
| C318 | 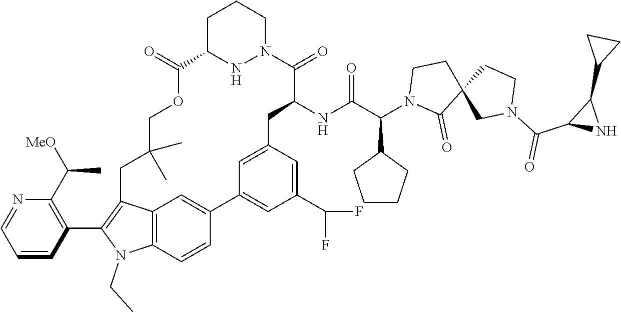 |
| C319 | 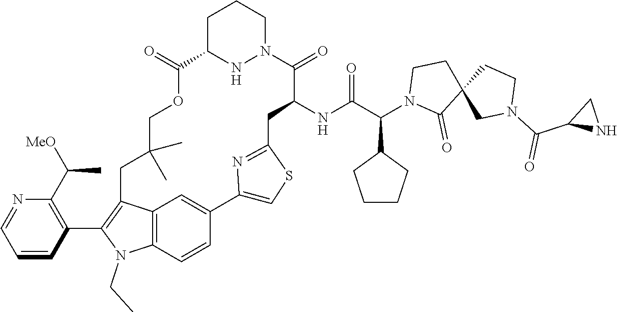 |
| C320 | 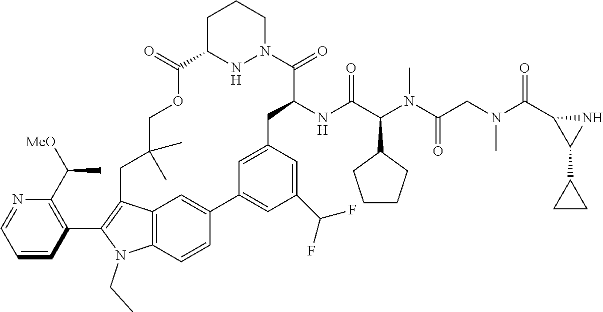 |
| C321 | 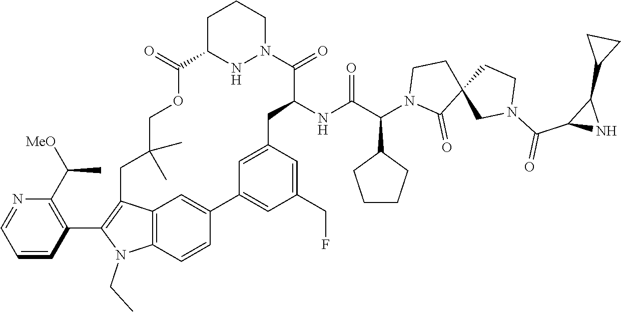 |
| C322* | 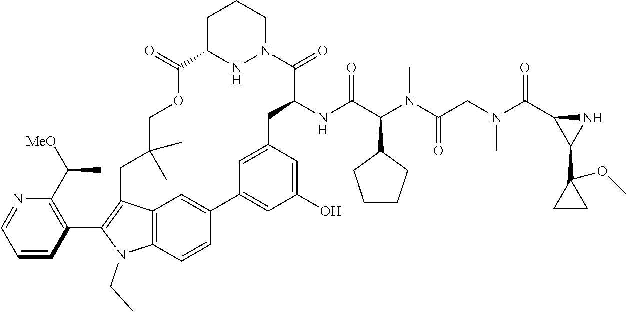 |
| C323* | 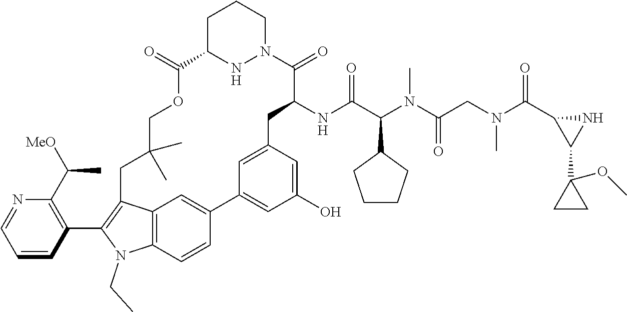 |
| C324 | 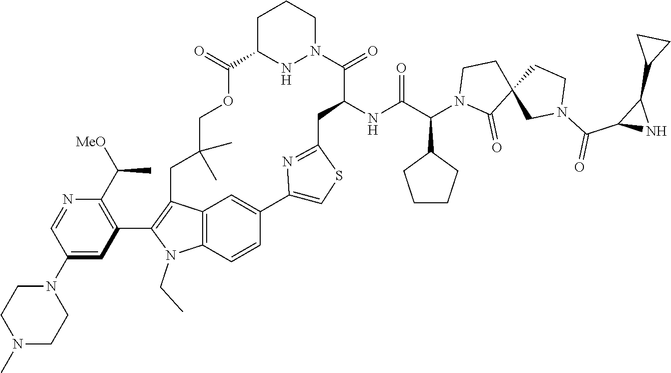 |
| C325 | 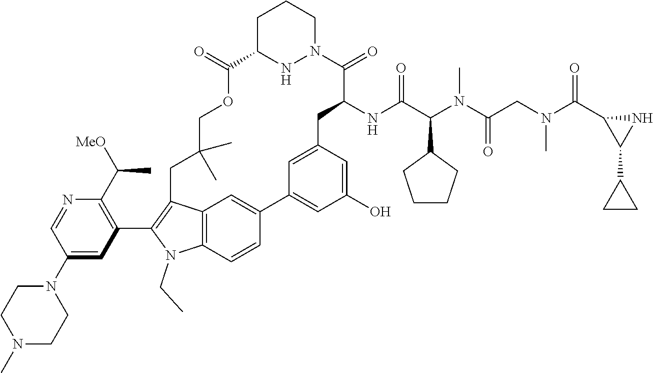 |
| C326 | 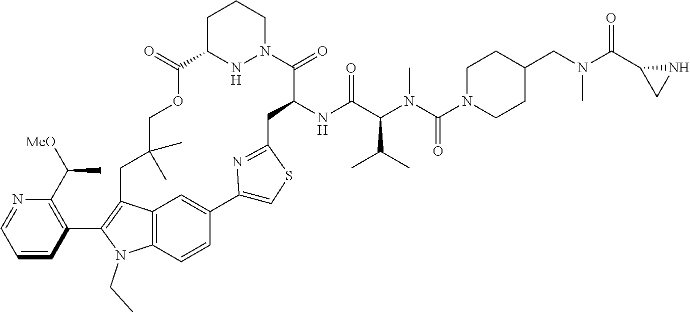 |
| C327 | 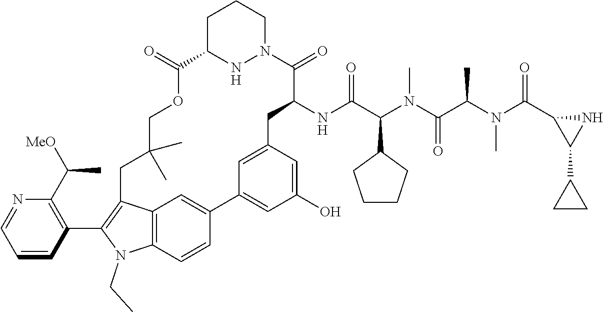 |
| C328 | 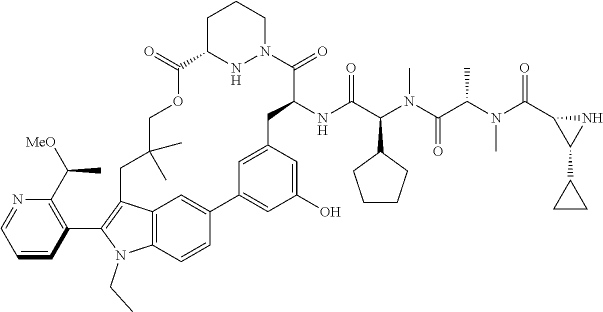 |
| C329 | 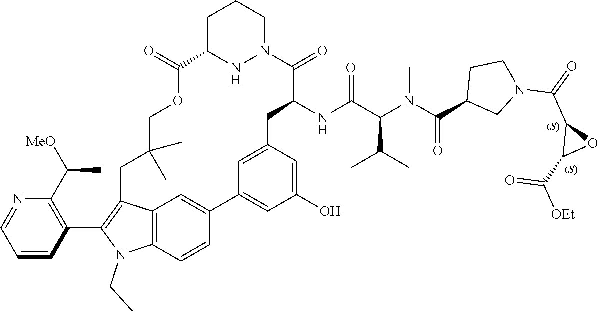 |
| C330 | 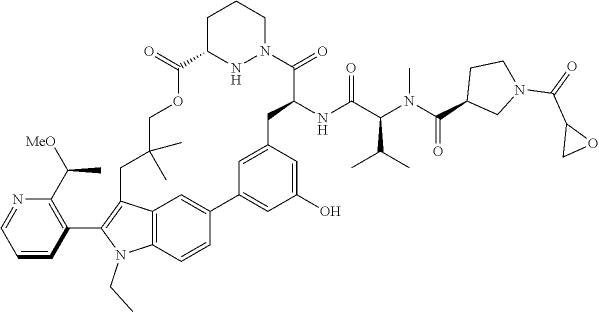 |
| C331 | 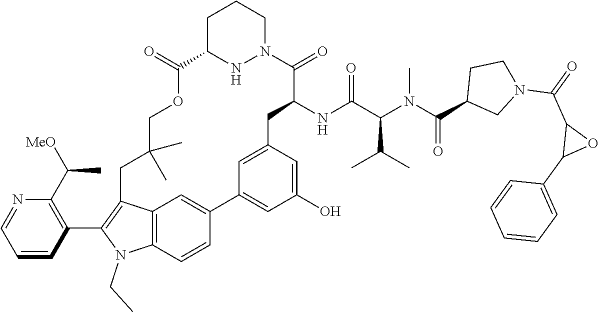 |
| C332 | 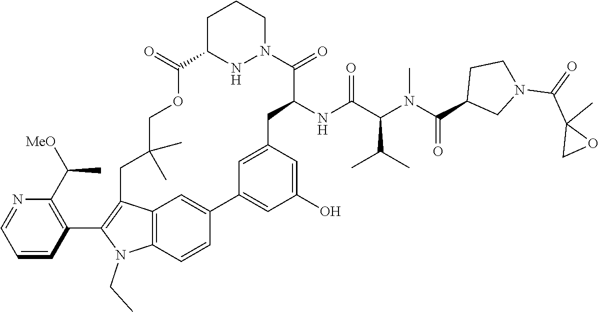 |

*Stereochemistry of the aziridine carbon is assumed.
Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.

In some embodiments, a compound of Table C2 is provided, or a pharmaceutically acceptable salt thereof. In some embodiments, the RAS(ON) inhibitor is selected from Table C2, or a pharmaceutically acceptable salt or atropisomer thereof

TABLE C2
Certain Compounds of the Present Invention

| Ex# | Structure |
|---|---|
| CB5 | 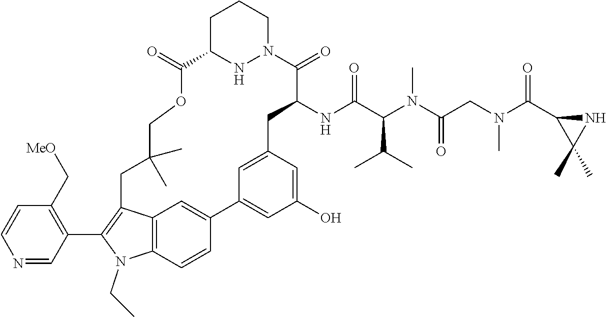 |
| CB6 | 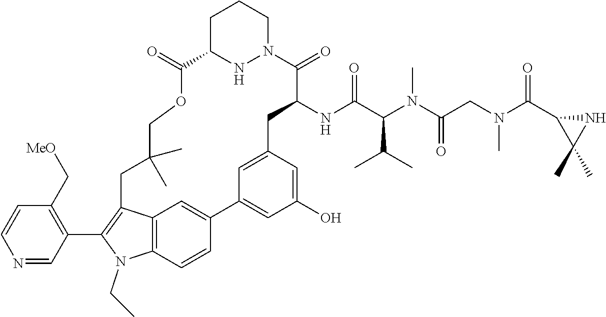 |
| CB8 | 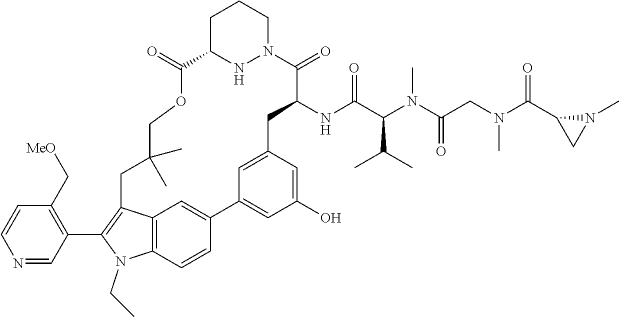 |
| CB9 | 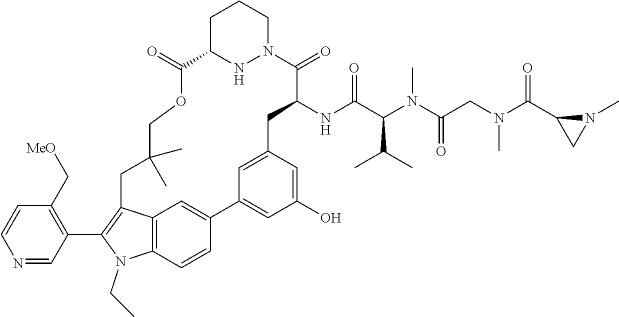 |
| CB16 | 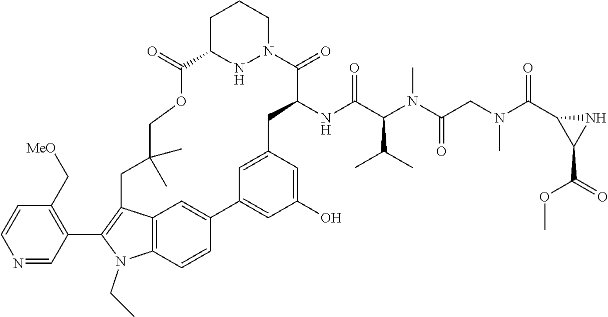 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C(=O)OC)C(=O)N1CCC[C@H](N1)C(=O)OCC(C)(C)C2] |
| CB19 | 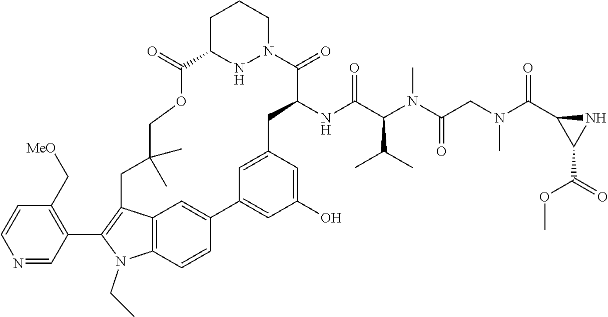 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C(=O)OC)C(=O)N1CCC[C@H](N1)C(=O)OCC(C)(C)C2] |
| CB29 | 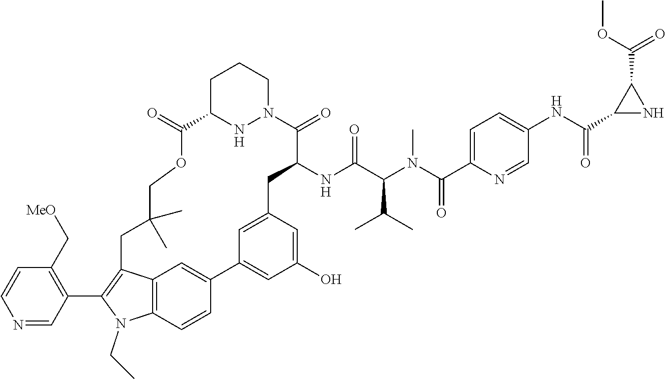 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@H]3C(=O)OC)cn1)C(=O)N1CCC[C@H](N1)C(=O)OCC(C)(C)C2] |
| CB30 | 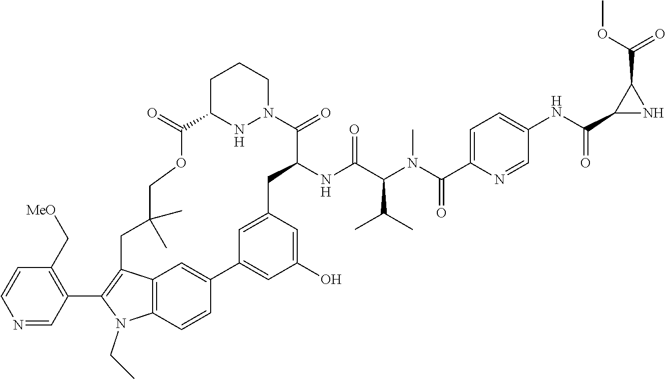 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@@H]3C(=O)OC)cn1)C(=O)N1CCC[C@H](N1)C(=O)OCC(C)(C)C2] |
| CB31 | 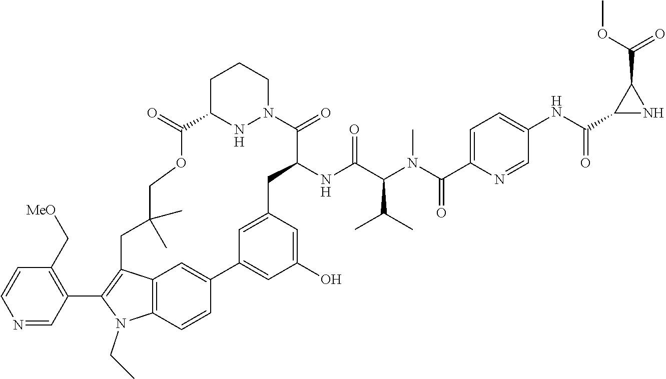 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@@H]3C(=O)OC)cn1)C(=O)N1CCC[C@H](N1)C(=O)OCC(C)(C)C2] |
| CB32 | 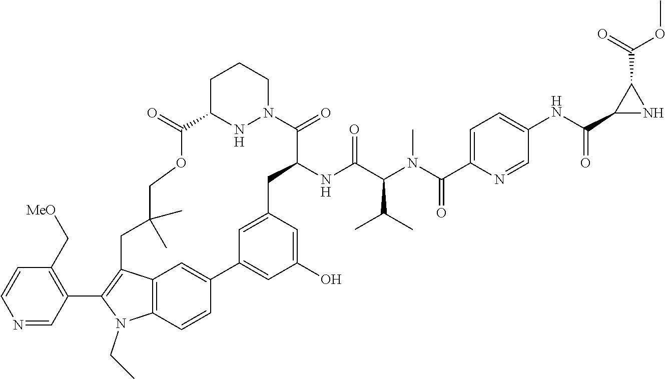 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@H]3C(=O)OC)cn1)C(=O)N1CCC[C@H](N1)C(=O)OCC(C)(C)C2] |
| CB35 | 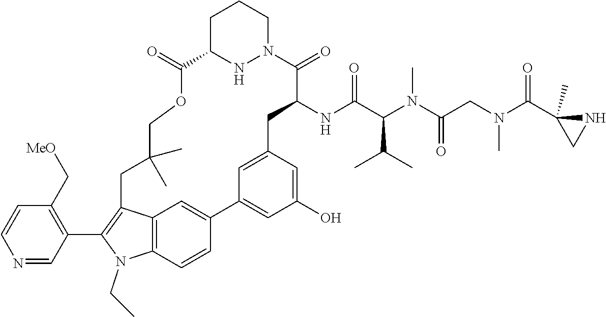 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@]1(C)CN1)C(=O)N1CCC[C@H](N1)C(=O)OCC(C)(C)C2] |
| CB36 | 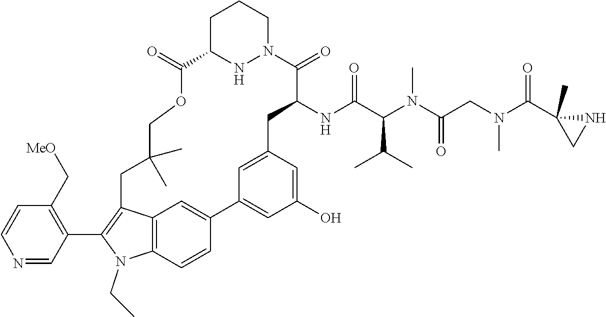 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@]1(C)CN1)C(=O)N1CCC[C@H](N1)C(=O)OCC(C)(C)C2] |
| CB37 | 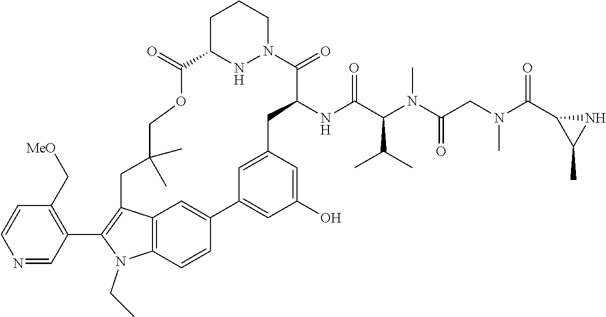 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C)C(=O)N1CCC[C@H](N1)C(=O)OCC(C)(C)C2] |
| CB38 | 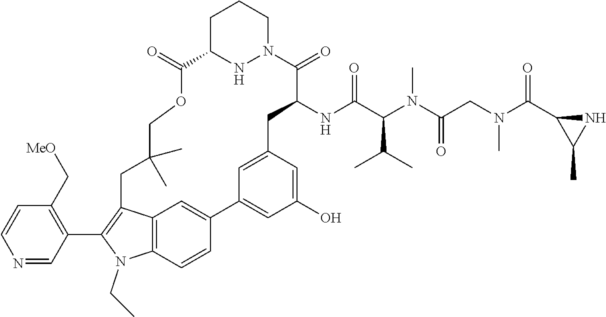 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C)C(=O)N1CCC[C@H](N1)C(=O)OCC(C)(C)C2] |
| CB40 | 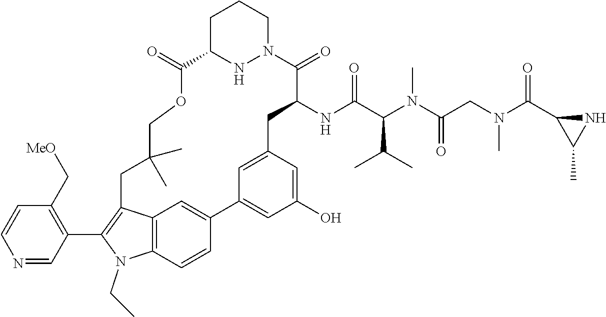 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C)C(=O)N1CCC[C@H](N1)C(=O)OCC(C)(C)C2] |
| CB41 | 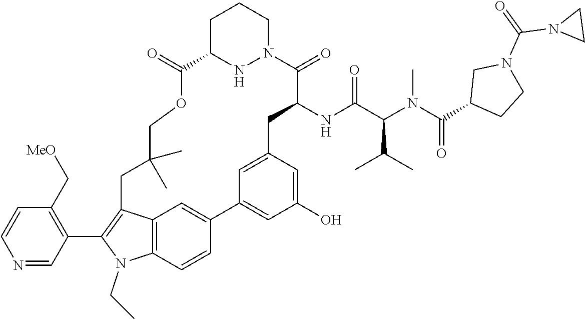 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3CC3)C1)C(=O)N1CCC[C@H](N1)C(=O)OCC(C)(C)C2] |
| CB42 | 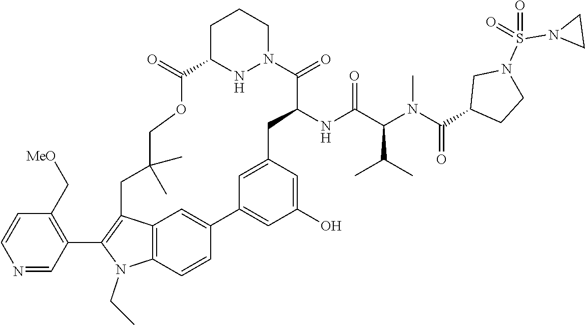 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3CC3)C1)C(=O)N1CCC[C@H](N1)C(=O)OCC(C)(C)C2] |
| CB43 | 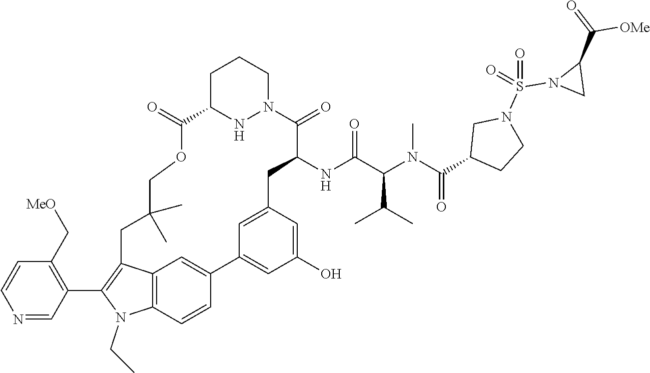 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@@H]3C(=O)OC)C1)C(=O)N1CCC[C@H](N1)C(=O)OCC(C)(C)C2] |
| CB44 | 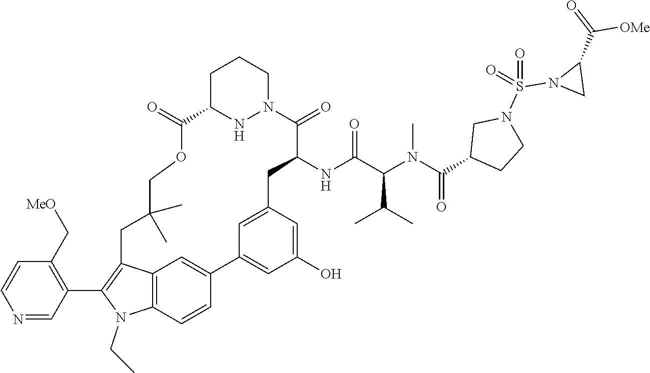 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)N3C[C@H]3C(=O)OC)C1)C(=O)N1CCC[C@H](N1)C(=O)OCC(C)(C)C2] |
| CB46 | 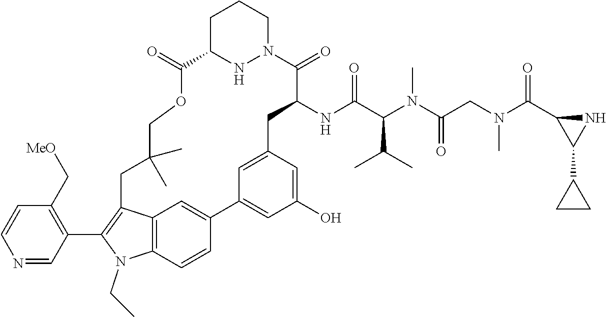 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C1CC1)C(=O)N1CCC[C@H](N1)C(=O)OCC(C)(C)C2] |
| CB48 | 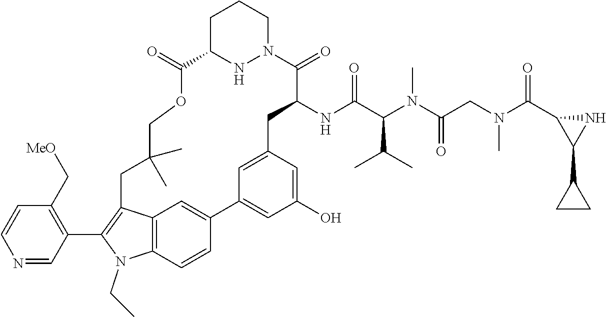 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C1CC1)C(=O)N1CCC[C@H](N1)C(=O)OCC(C)(C)C2] |
| CB51 | 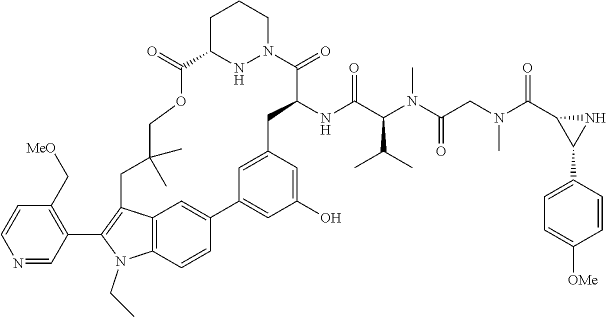 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccc(OC)cc1)C(=O)N1CCC[C@H](N1)C(=O)OCC(C)(C)C2] |
| CB53 | 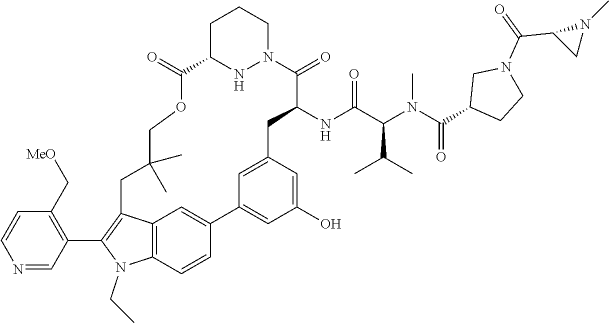 |
| CB54 | 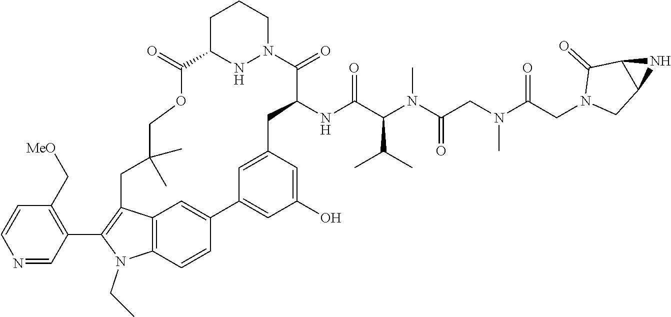 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@@H]3N[C@@H]3C1=O)C(=O)N1CCC[C@H](N1)C(=O)OCC(C)(C)C2] |
| CB55 | 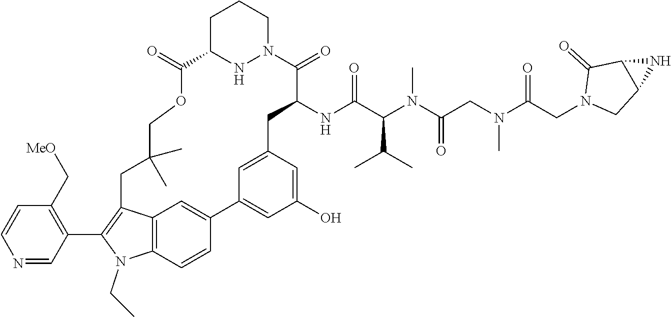 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@H]3N[C@H]3C1=O)C(=O)N1CCC[C@H](N1)C(=O)OCC(C)(C)C2] |
| CB56 | 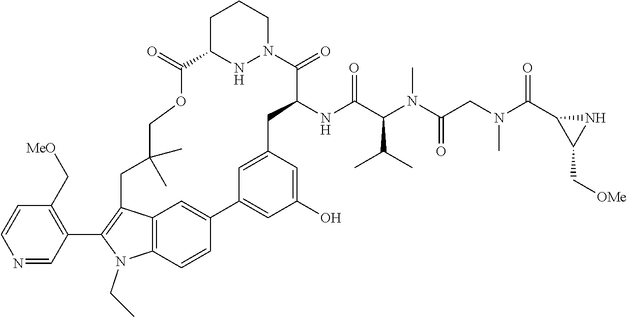 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1COC)C(=O)N1CCC[C@H](N1)C(=O)OCC(C)(C)C2] |
| CB57 | 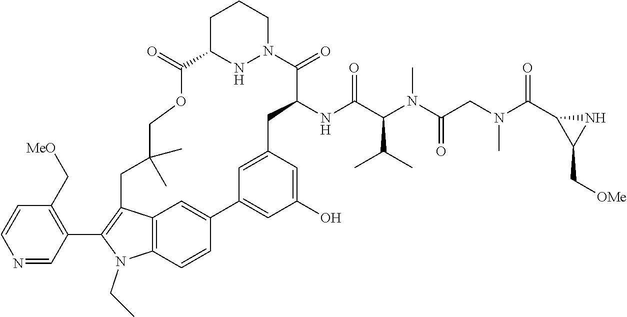 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1COC)C(=O)N1CCC[C@H](N1)C(=O)OCC(C)(C)C2] |
| CB58 | 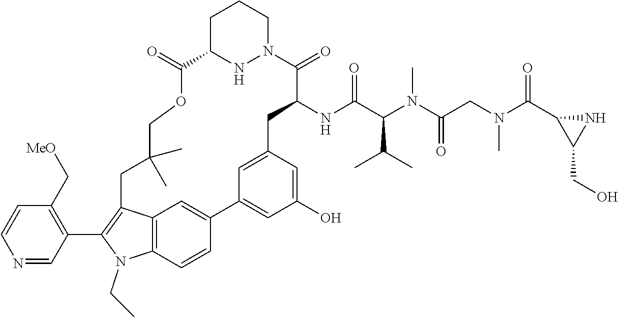 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1CO)C(=O)N1CCC[C@H](N1)C(=O)OCC(C)(C)C2] |
| CB59 | 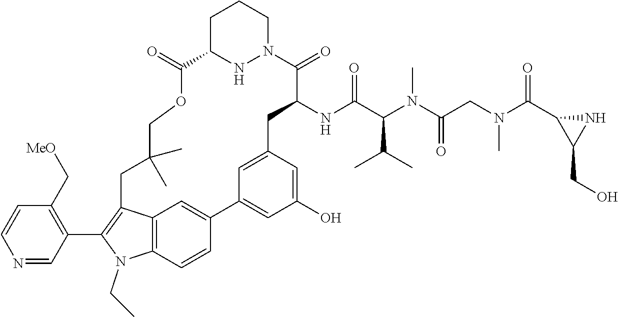 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1CO)C(=O)N1CCC[C@H](N1)C(=O)OCC(C)(C)C2] |
| CB60 | 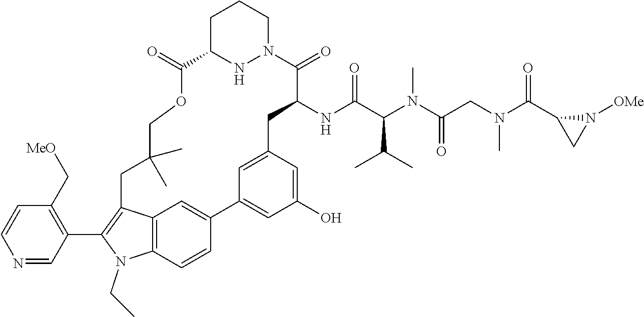 |
| CB61 | 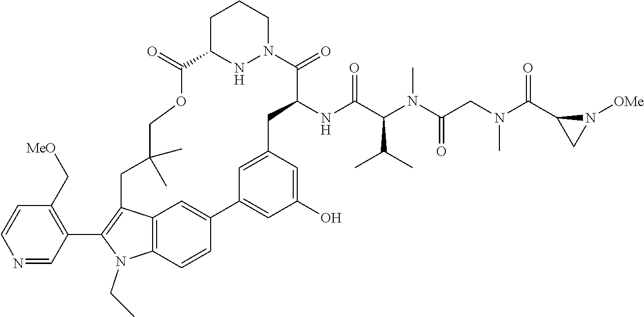 |
| CB62 | 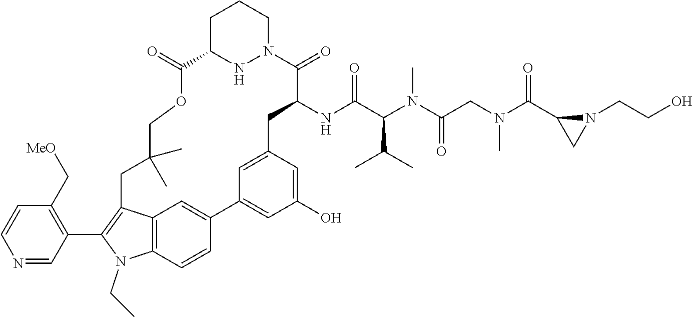 |
| CB63 | 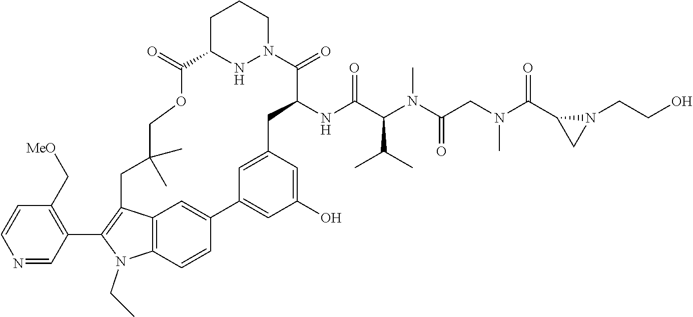 |
| CB73 | 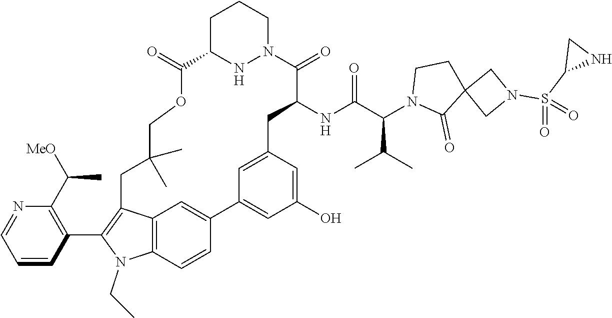 |
| CB74 | 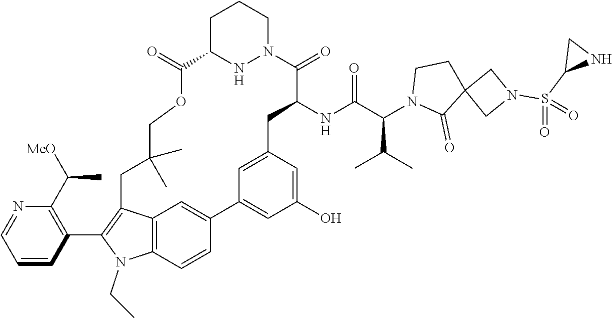 |
| CB76 | 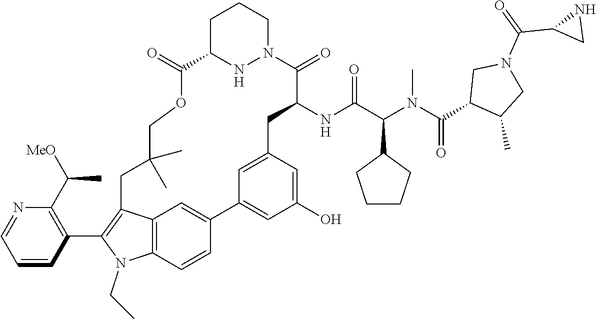 |
| CB78 | 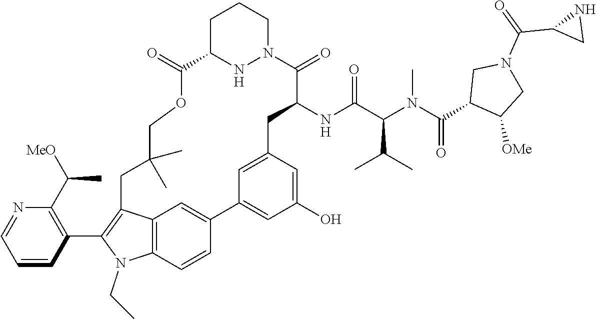 |
| CB79 | 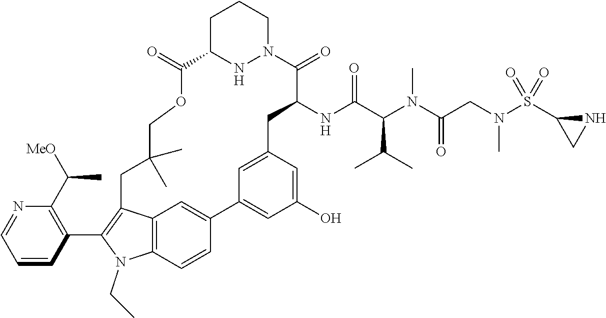 |
| CB80 | 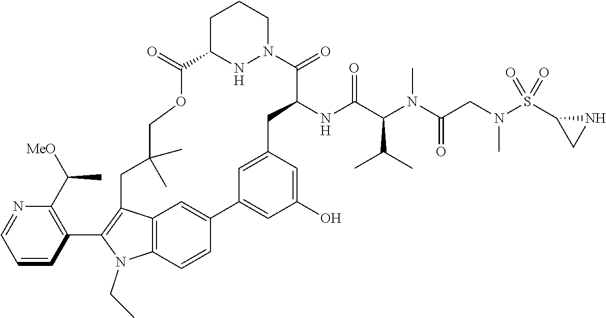 |
| CB83 | 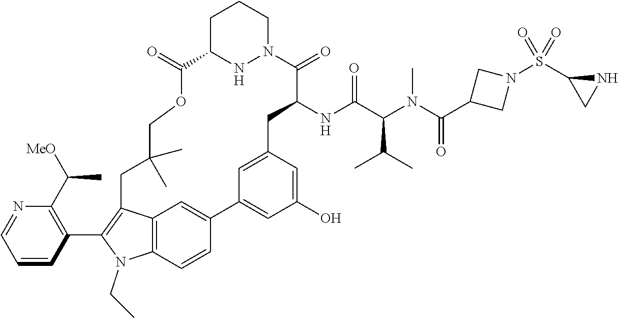 |
| CB84 | 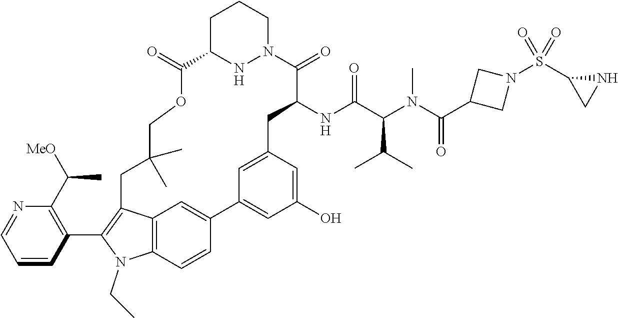 |
| CB87 | 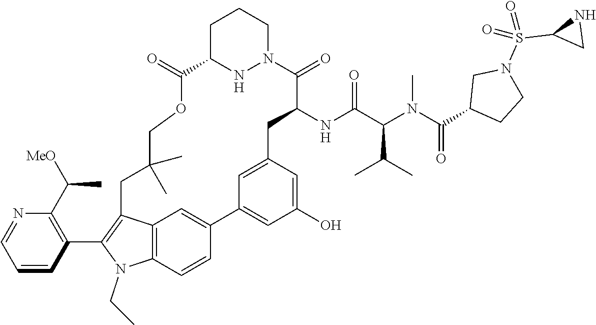 |
| CB88 | 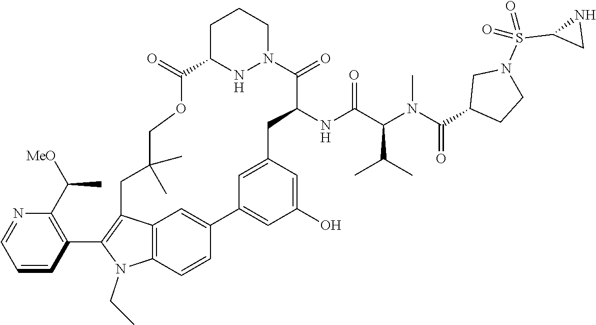 |
| CB91 | 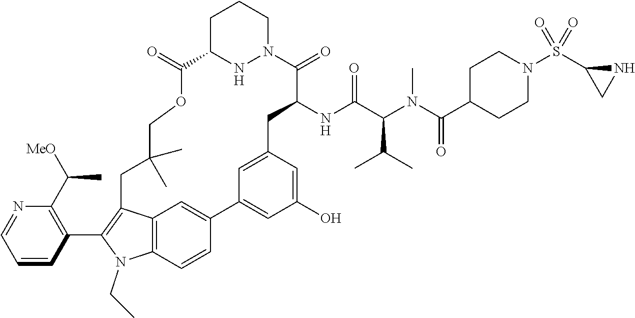 |
| CB92 | 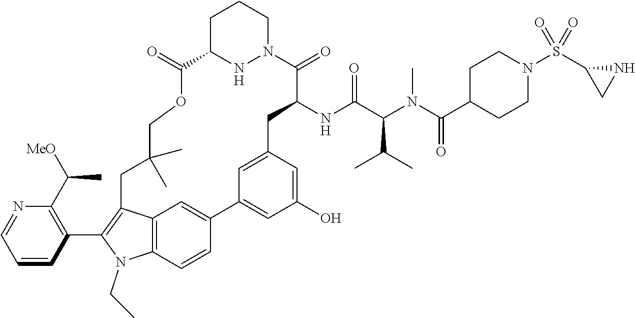 |
| CB97 | 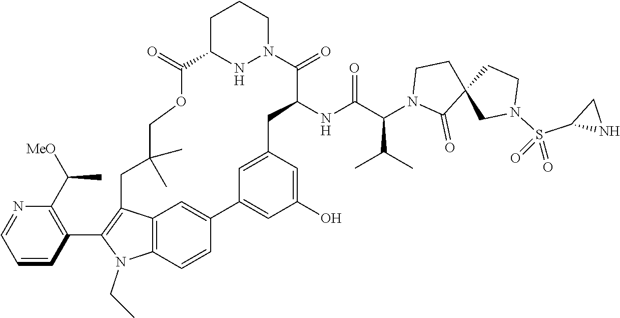 |
| CB98 | 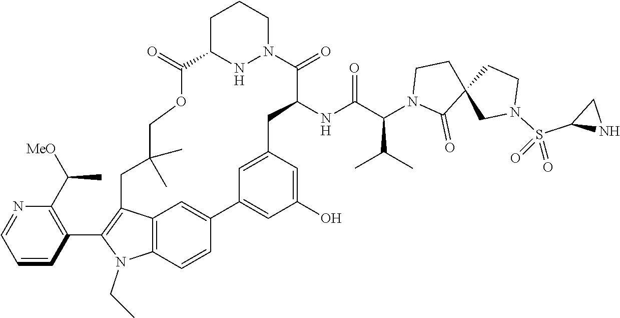 |
| CB101 | 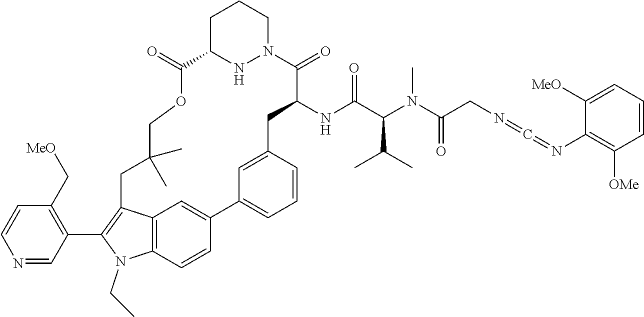 [SMILES: CCn1c(-c2cnccc2COC)c2c3cc(ccc31)-c1cccc(c1)C[C@H](NC(=O)[C@H](C(C)C)N(C)C(=O)CN=C=Nc1c(OC)cccc1OC)C(=O)N1CCC[C@H](N1)C(=O)OCC(C)(C)C2] |
| CB108 | 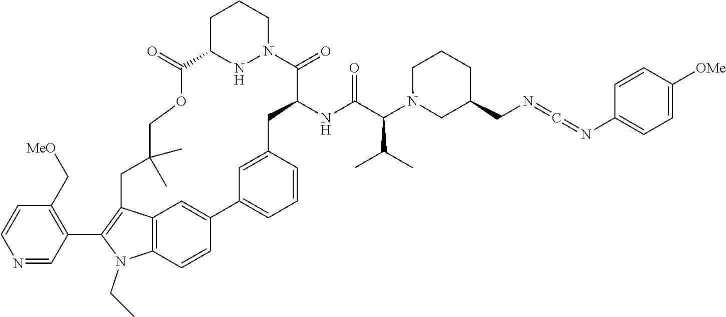 [SMILES: CCn1c(-c2cnccc2COC)c2c3cc(ccc31)-c1cccc(c1)C[C@H](NC(=O)[C@H](C(C)C)N1CCC[C@@H](CN=C=Nc3ccc(OC)cc3)C1)C(=O)N1CCC[C@H](N1)C(=O)OCC(C)(C)C2] |
| CB109 | 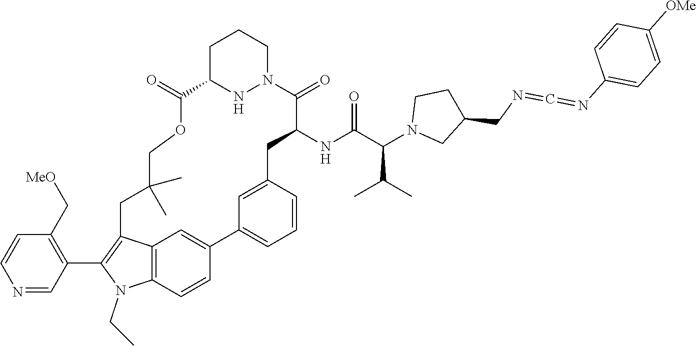 [SMILES: CCn1c(-c2cnccc2COC)c2c3cc(ccc31)-c1cccc(c1)C[C@H](NC(=O)[C@H](C(C)C)N1CC[C@@H](CN=C=Nc3ccc(OC)cc3)C1)C(=O)N1CCC[C@H](N1)C(=O)OCC(C)(C)C2] |
| CB113 | 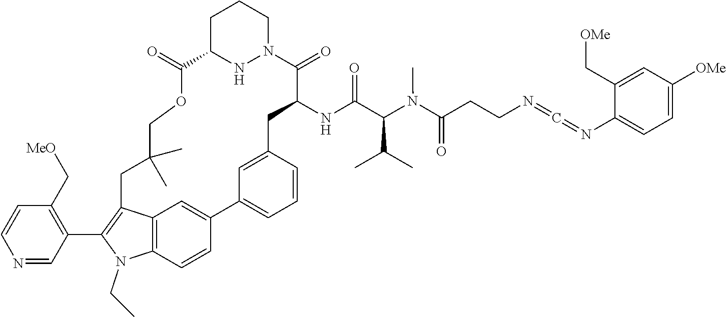 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1COC)C(=O)N1CCC[C@H](N1)C(=O)OCC(C)(C)C2] |
| CB116 | 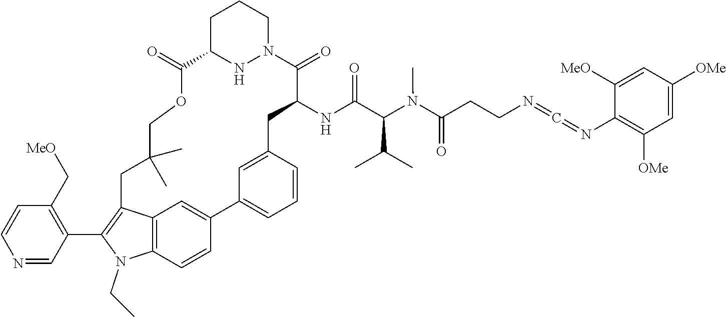 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cc(OC)cc1OC)C(=O)N1CCC[C@H](N1)C(=O)OCC(C)(C)C2] |
| CB117 | 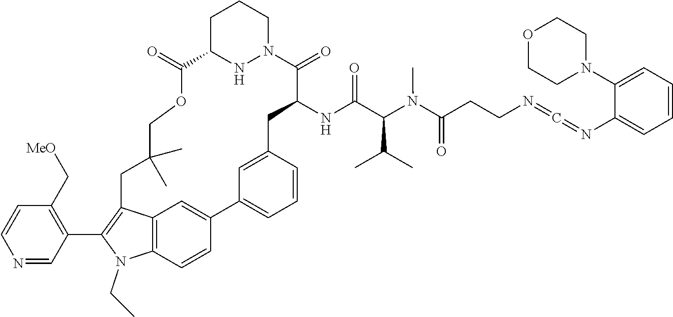 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1N1CCOCC1)C(=O)N1CCC[C@H](N1)C(=O)OCC(C)(C)C2] |
| CB118 | 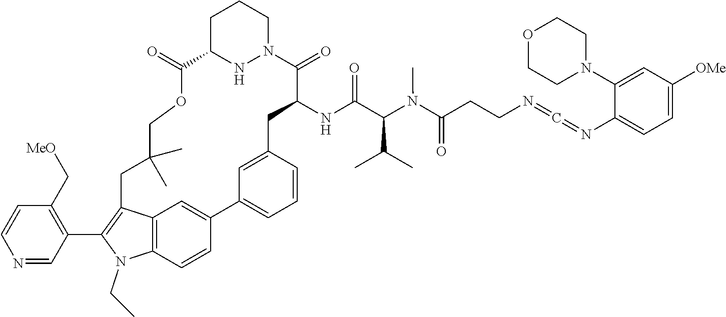 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1N1CCOCC1)C(=O)N1CCC[C@H](N1)C(=O)OCC(C)(C)C2] |
| CB119 | 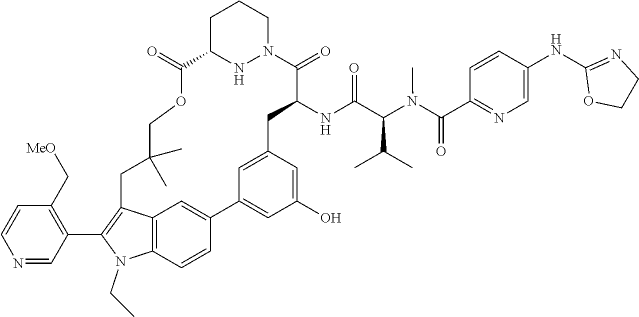 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC3=NCCO3)cn1)C(=O)N1CCC[C@H](N1)C(=O)OCC(C)(C)C2] |
| CB120 | 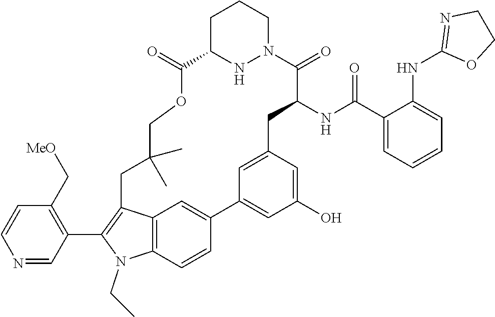 [SMILES: CCn1c(-c2cnccc2COC)c2c3cc(ccc31)-c1cc(O)cc(c1)C[C@H](NC(=O)c1ccccc1NC1=NCCO1)C(=O)N1CCC[C@H](N1)C(=O)OCC(C)(C)C2] |
| CB122 | 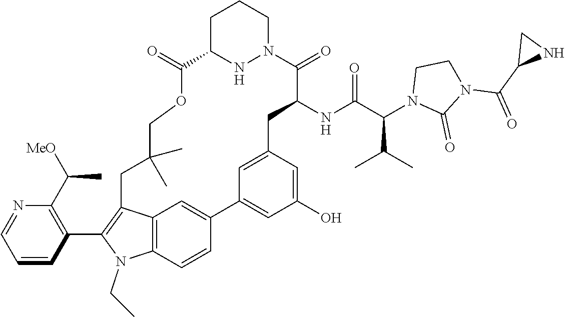 |
| CB123 | 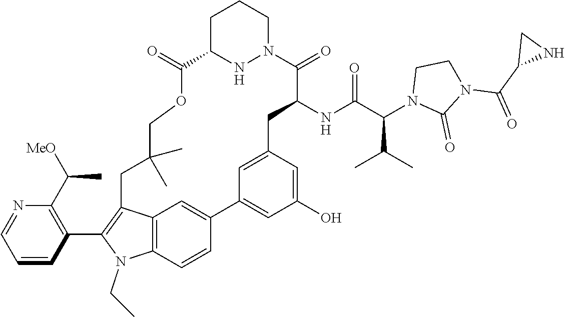 |

Note
that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, the RAS(ON) inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below and in WO 2021/091967.

Compounds of Table C1 herein were prepared using methods disclosed herein or were prepared using methods disclosed herein combined with the knowledge of one of skill in the art. Compounds of Table C2 may be prepared using methods disclosed herein or may be prepared using methods disclosed herein combined with the knowledge of one of skill in the art.

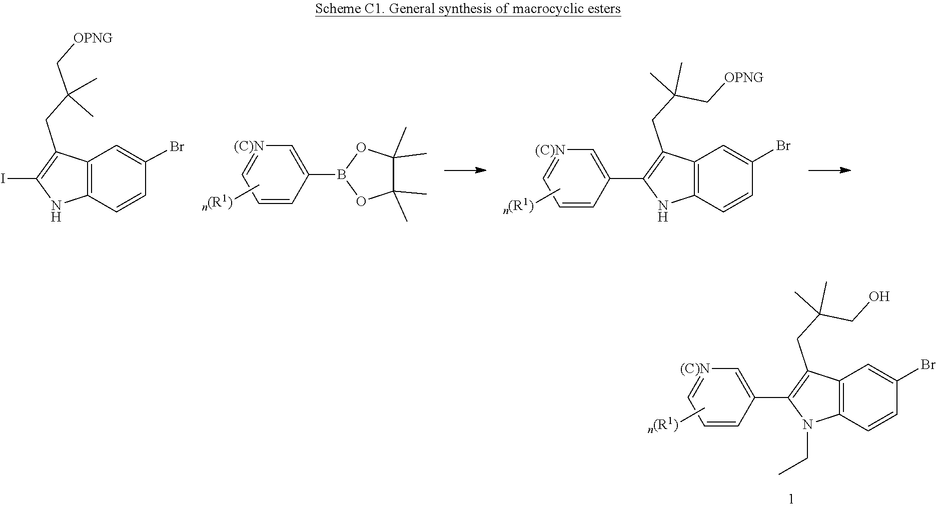

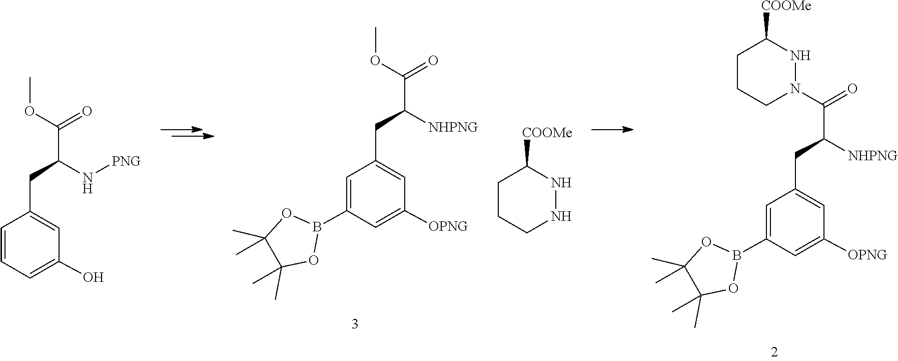

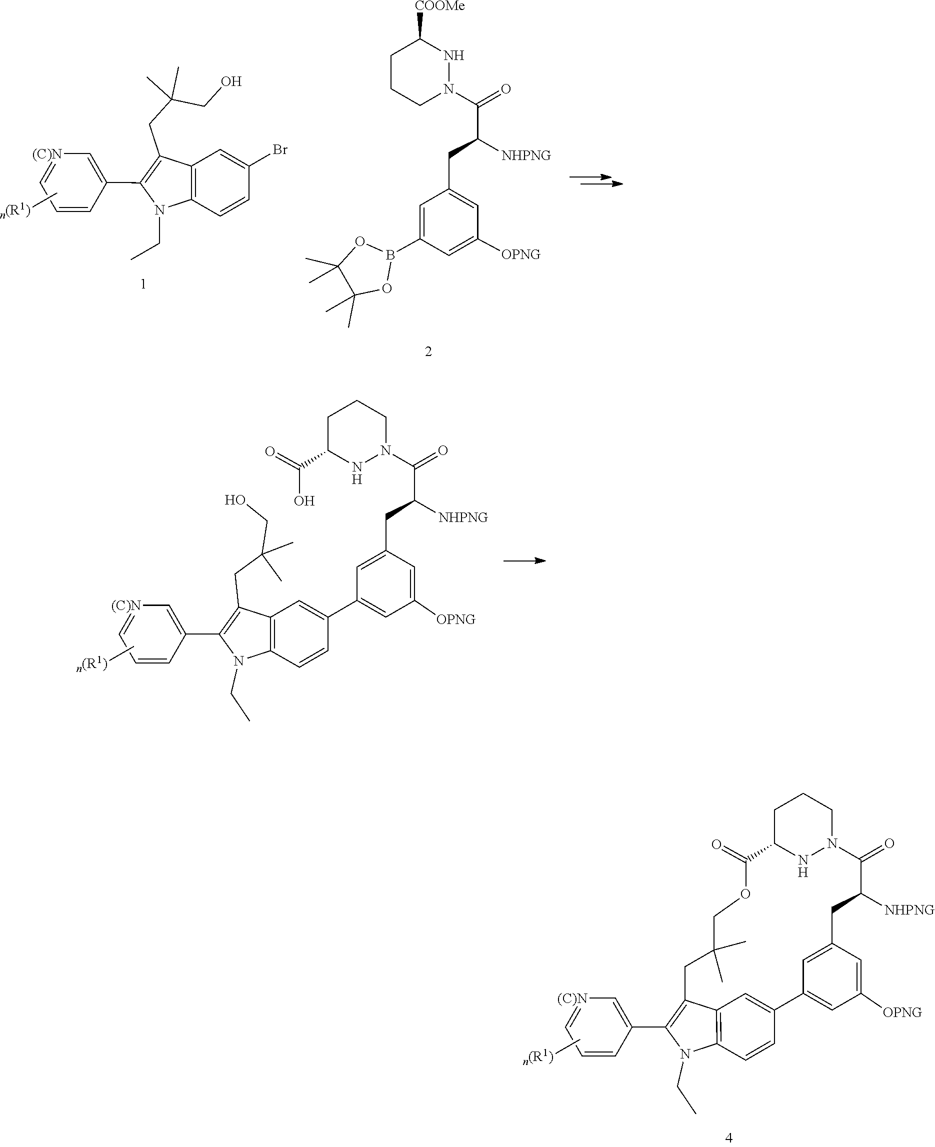

A general synthesis of macrocyclic esters is outlined in Scheme C1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl (S)-hexahydropyridazine-3-carboxylate.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of Pd catalyst followed by hydrolysis and macrolactonization steps to result in an appropriately protected macrocyclic intermediate (4). Additional deprotection or functionalization steps are required to produce a final compound. For example, a person of skill in the art would be able to install into a macrocyclic ester a desired —B-L-W group of a compound of Formula (CI), where B, L and W are defined herein, including by using methods exemplified in certain Schemes below and in the Example section herein.

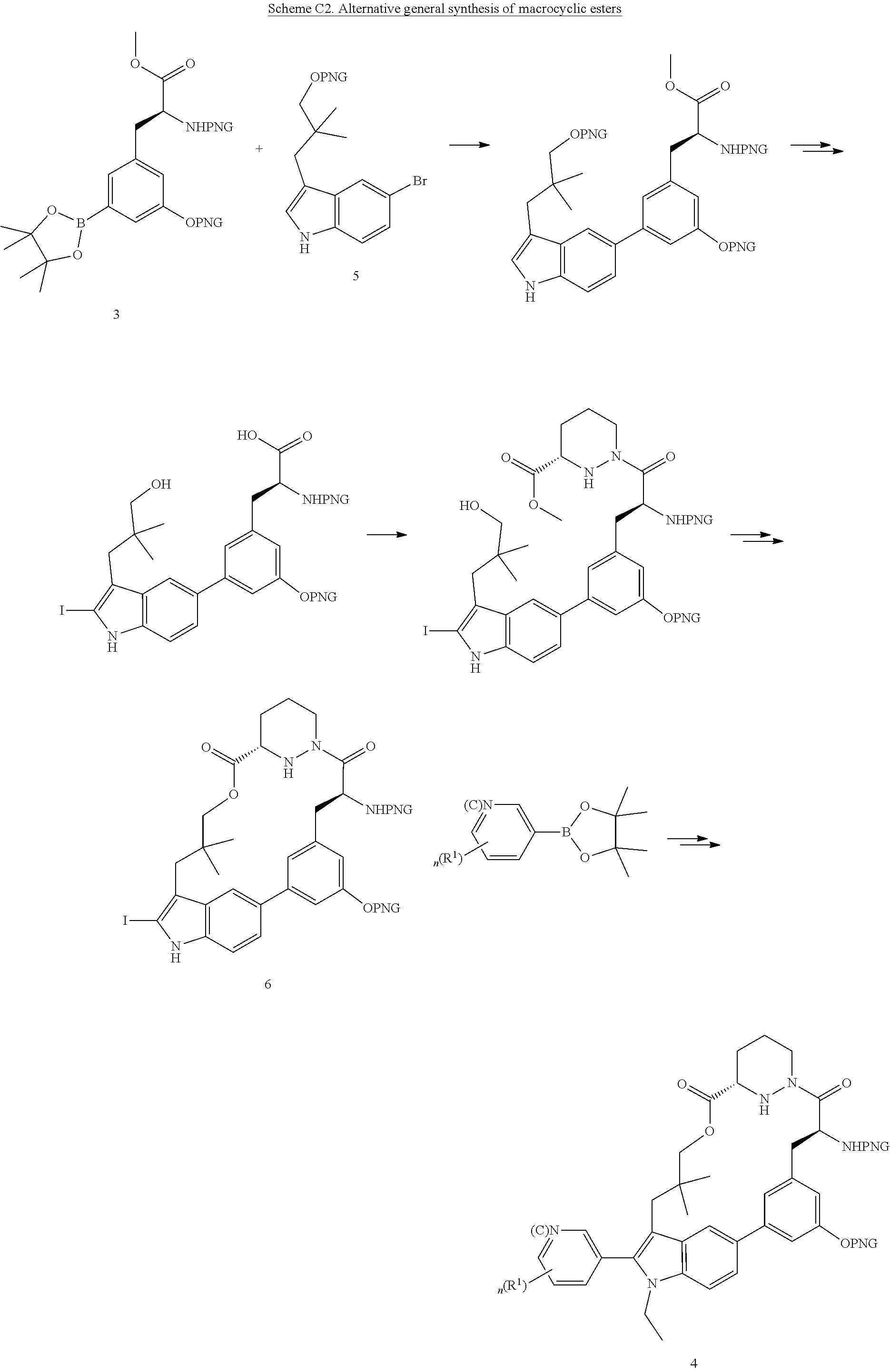

Alternatively, macrocyclic esters can be prepared as described in Scheme C2. An appropriately protected bromo-indolyl (5) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (6). Coupling in the presence of Pd catalyst with an appropriately substituted boronic ester can yield fully a protected macrocycle (4). Additional deprotection or functionalization steps are required to produce a final compound. For example, a person of skill in the art would be able to install into a macrocyclic ester a desired —B-L-W group of a compound of Formula (CI), where B, L and W are defined herein, including by using methods exemplified in certain Schemes below and in the Example section herein.

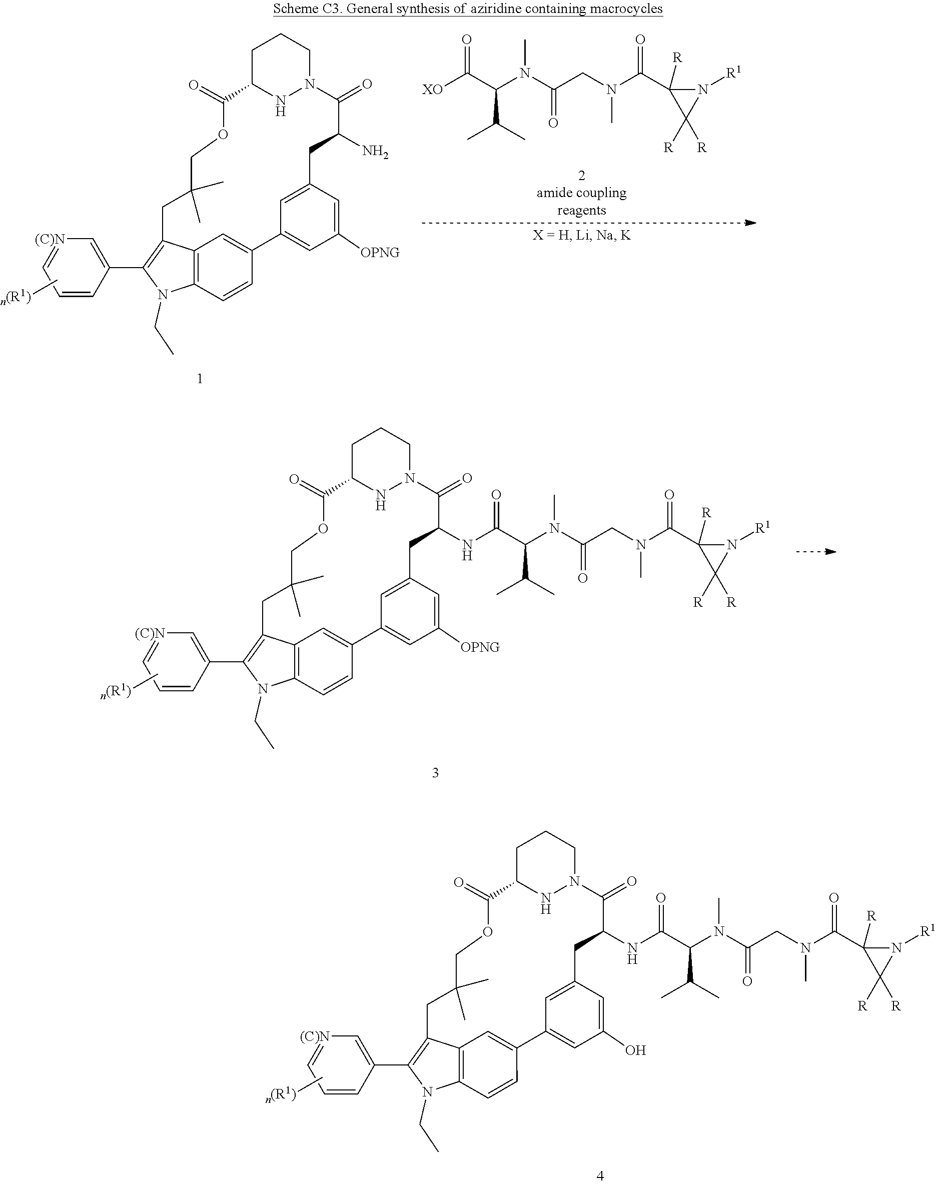

As shown in Scheme C3, compounds of this type may be prepared by the reaction of an appropriate amine (1) with an aziridine containing carboxylic acid (2) in the presence of standard amide coupling reagents, followed by deprotection of the aziridine, if R¹ is a protecting group, and deprotection of the phenol, if required, to produce the final compound (4).

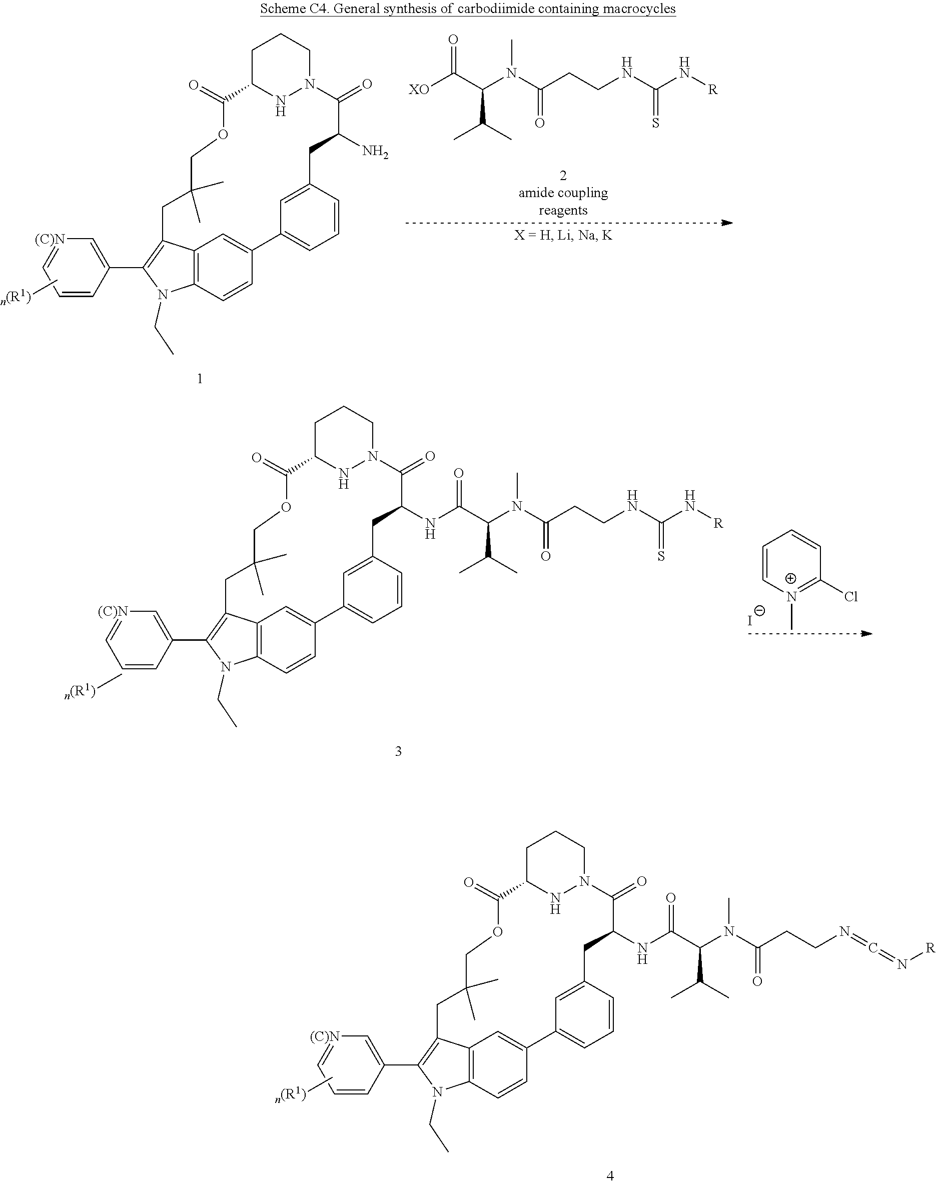

As shown in Scheme C4, compounds of this type may be prepared by the reaction of an appropriate amine (1) with a thiourea containing carboxylic acid (2) in the presence of standard amide coupling reagents, followed by conversion of the thiourea (3) to a carbodiimide (4) in the presence of 2-chloro-1-methylpyridin-1-ium iodide.

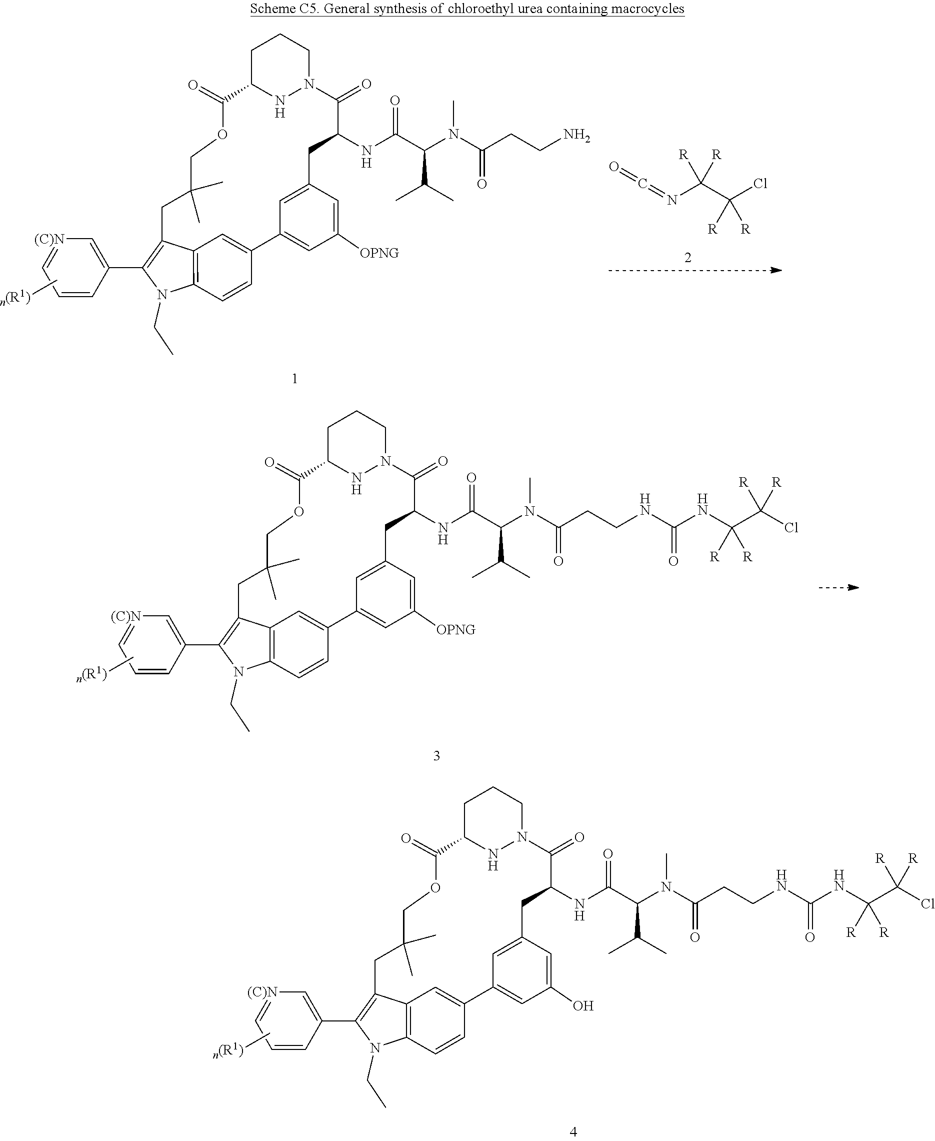

As shown in Scheme C5, compounds of this type may be prepared by the reaction of an appropriate amine (1) with an isocyanate (2) under basic conditions, followed by deprotection of the phenol, if required, to produce the final compound (4)

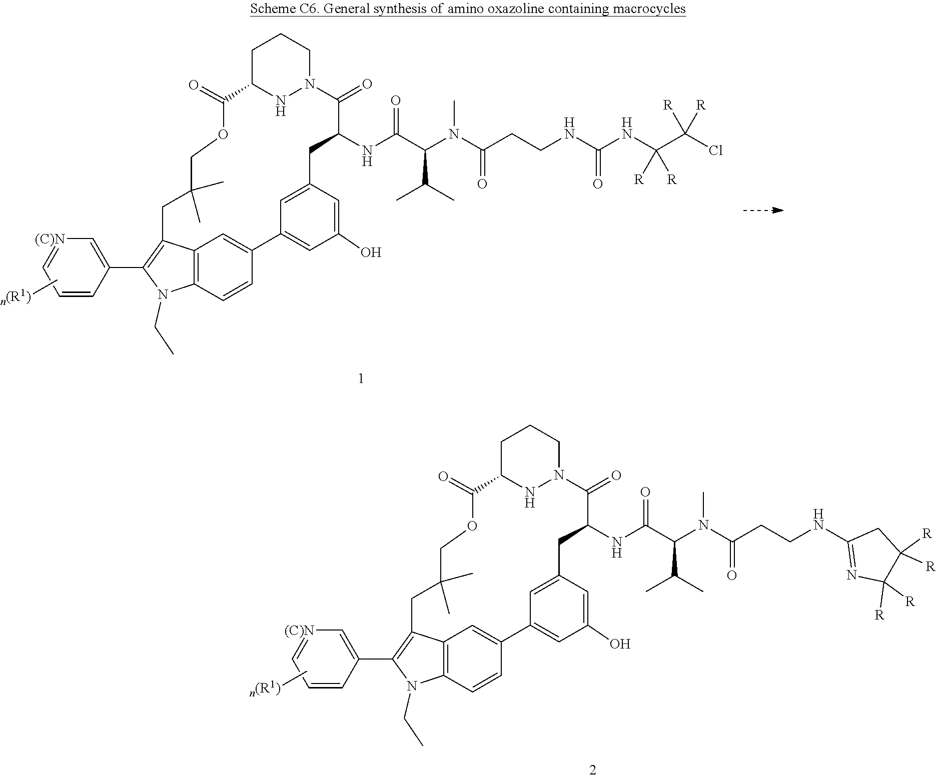

As shown in Scheme C6, compounds of this type may be prepared by cyclization of an appropriate chloroethyl urea (1) under elevated temperatures to produce the final compound (2).

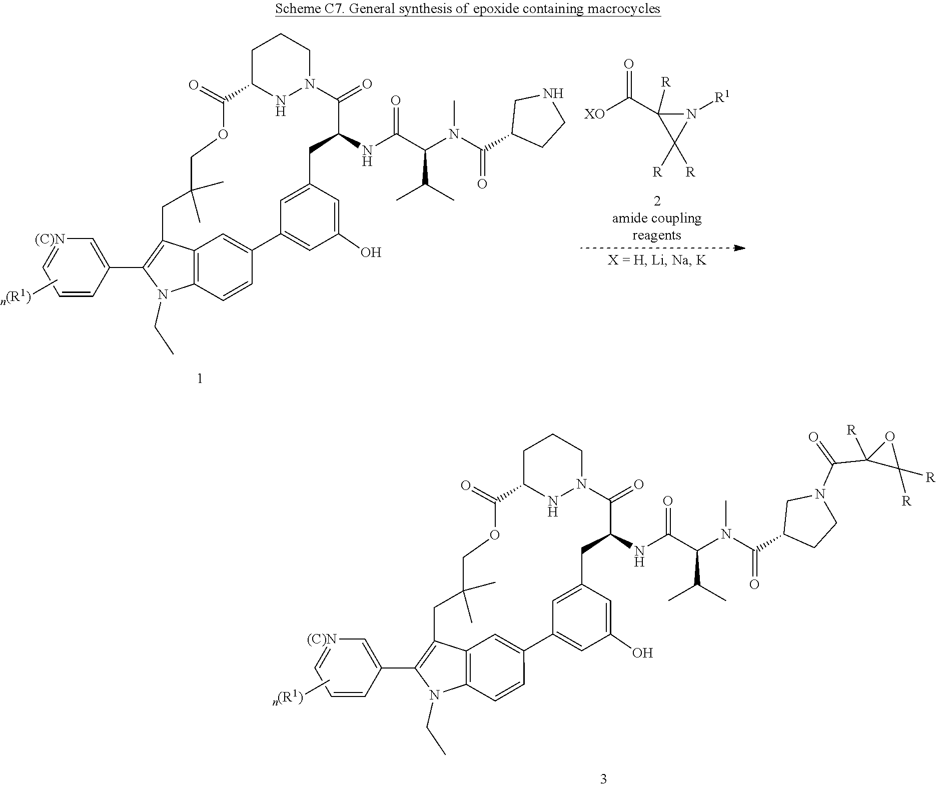

As shown in Scheme C7, compounds of this type may be prepared by the reaction of an appropriate amine (1) with an epoxide containing carboxylic acid (2) in the presence of standard amide coupling reagents to produce the final compound (3).

In addition, compounds of the disclosure can be synthesized using the methods described in the WO 2021/091967, together with synthetic methods known in the art of synthetic organic chemistry, or variations thereon as appreciated by those skilled in the art. These methods include but are not limited to those methods described in the WO 2021/091967. For example, a person of skill in the art would be able to install into a macrocyclic ester a desired —B-L-W group of a compound of Formula (CI), where B, L and W are defined herein, including by using methods exemplified in certain Schemes above and in the Example section herein.

In some embodiments, the RAS inhibitor is a compound disclosed in WO 2022/060836.

In some embodiments, the RAS(ON) inhibitor is a compound, or a pharmaceutically acceptable salt thereof, having the structure of Formula DIa:

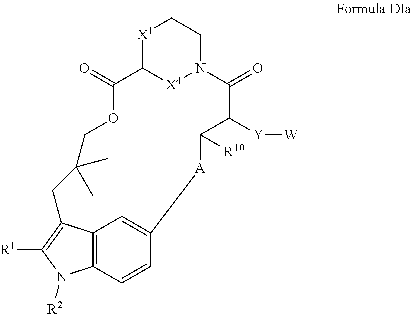

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;

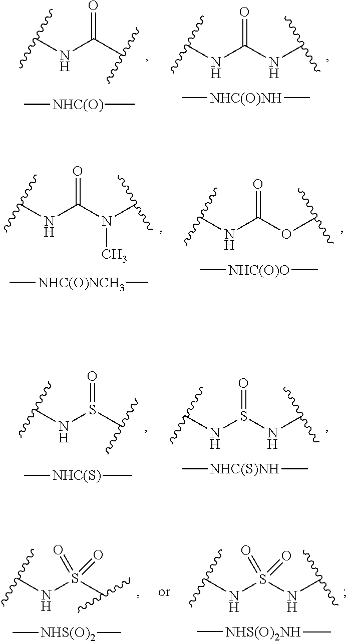

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

In some embodiments, the RAS(ON) inhibitor, or pharmaceutically acceptable salt thereof, has the structure of Formula DIa-2:

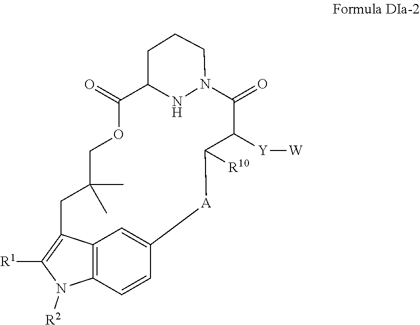

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;

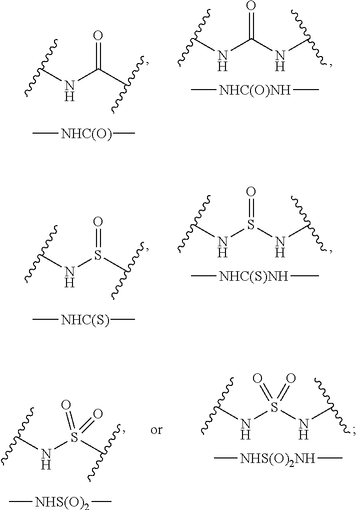

W is hydrogen, C₁-C₄ alkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen or optionally substituted C₁-C₆ heteroalkyl. In some embodiments, R¹⁰ is hydrogen.

In some embodiments of Formula DIa and subformula thereof, R¹ is optionally substituted 6 to 10-membered aryl or optionally substituted 5 to 10-membered heteroaryl. In some embodiments, R¹ is optionally substituted phenyl or optionally substituted pyridine.

In some embodiments of Formula DIa and subformula thereof, A is optionally substituted thiazole, optionally substituted triazole, optionally substituted morpholino, optionally substituted piperidinyl, optionally substituted pyridine, or optionally substituted phenyl. In some embodiments, A is optionally substituted thiazole, optionally substituted triazole, optionally substituted morpholino, or phenyl. In some embodiments, A is not an optionally substituted phenyl or benzimidazole. In some embodiments, A is not hydroxyphenyl.

In some embodiments of Formula DIa and subformula thereof, Y is —NHC(O)— or —NHC(O)NH—.

In some embodiments, the RAS(ON) inhibitor, or a pharmaceutically acceptable salt thereof, has the structure of Formula DIIa:

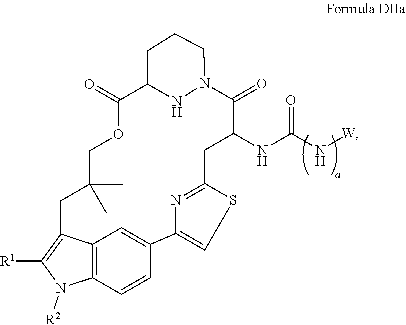

wherein W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
a is 0 or 1.

In some embodiments, the RAS(ON) inhibitor, or a pharmaceutically acceptable salt thereof, has the structure of Formula DIIa-1:

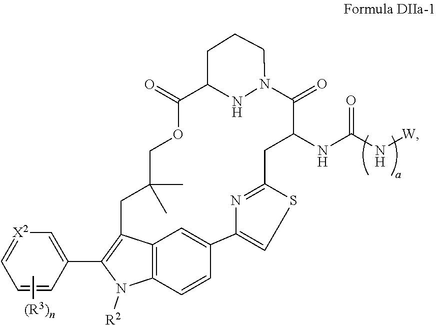

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIa-2:

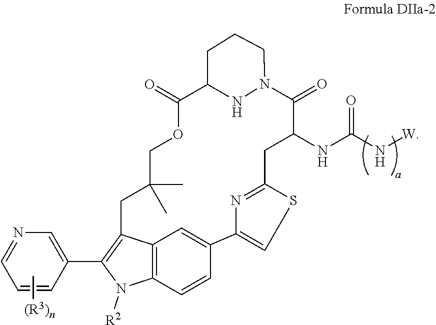

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIa-3:

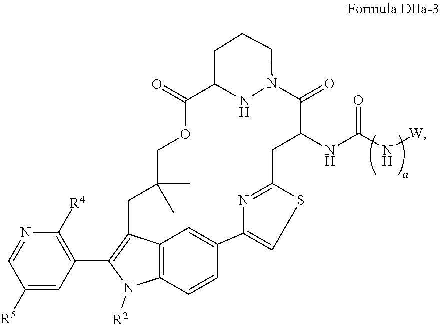

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-4:

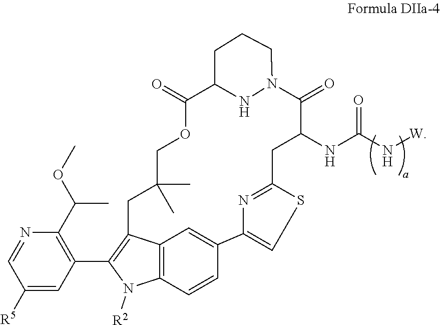

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-5:

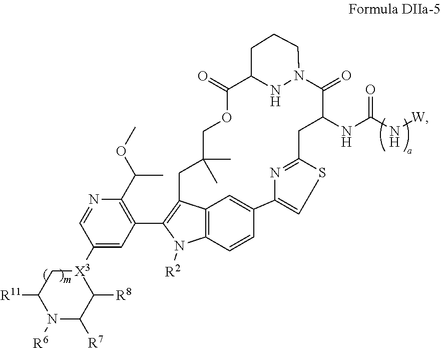

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is H. In some embodiments, X³ is N, m is 1, and R¹¹ is H.

In some embodiments, the RAS(ON) inhibitor, or a pharmaceutically acceptable salt thereof, has the structure of Formula DIIa-6:

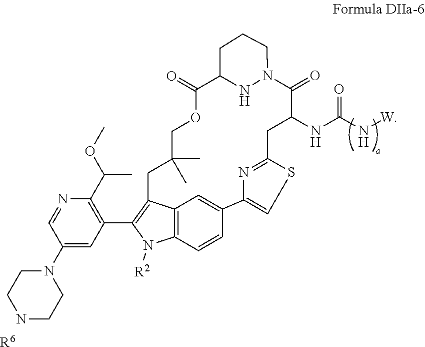

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-7:

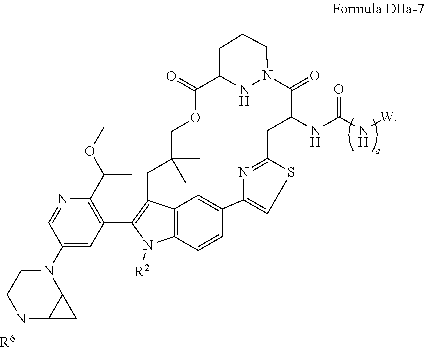

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIa-6 or DIIa-7), R⁶ is methyl.

In some embodiments, the RAS(ON) inhibitor, or a pharmaceutically acceptable salt thereof, has the structure of Formula DIIa-8 or Formula DIIa-9:

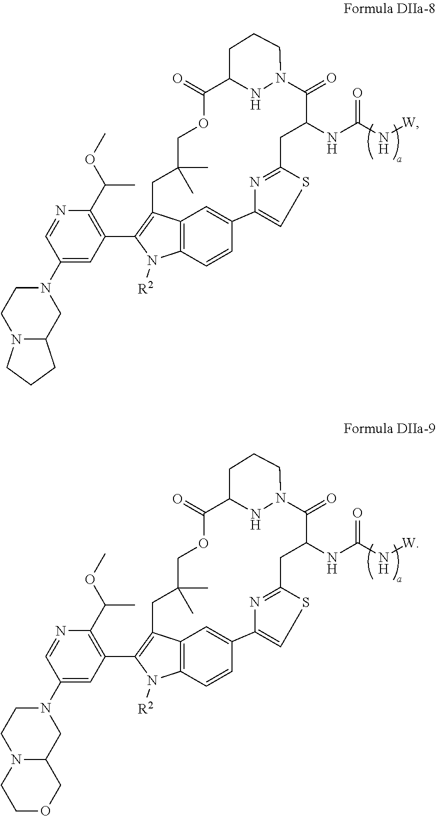

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl.

In some embodiments, the RAS(ON) inhibitor, or a pharmaceutically acceptable salt thereof, has the structure of Formula DIIIa:

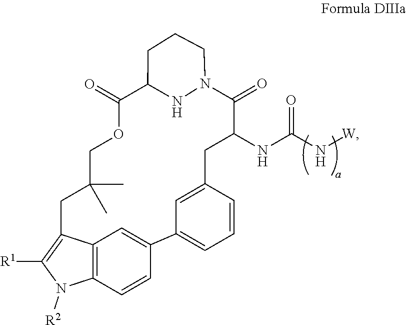

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS(ON) inhibitor, or a pharmaceutically acceptable salt thereof, has the structure of Formula DIIIa-1:

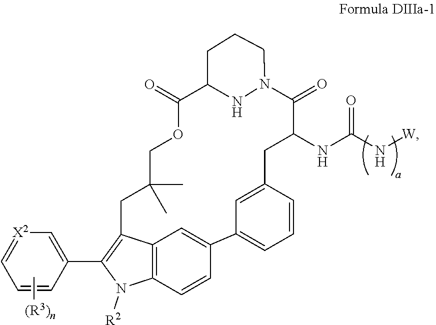

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIIa-2:

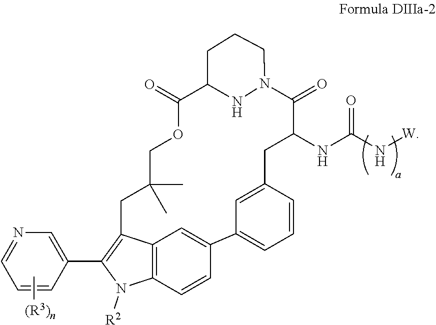

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIIa-3:

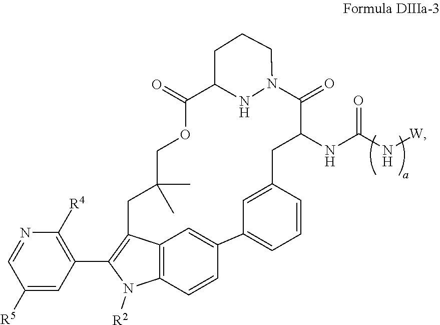

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-4:

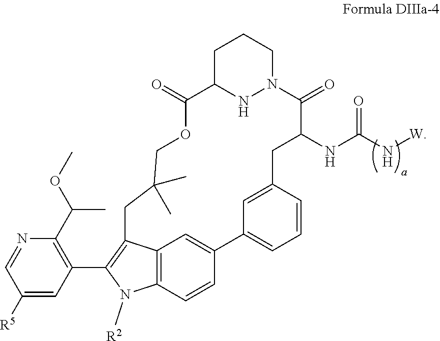

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-5:

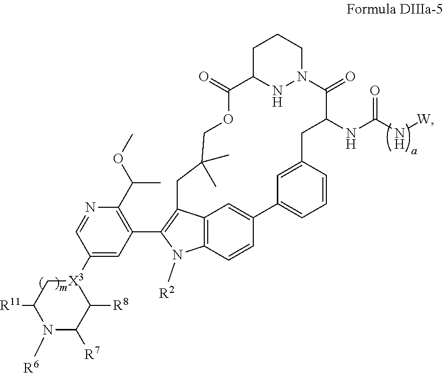

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIIIa-6:

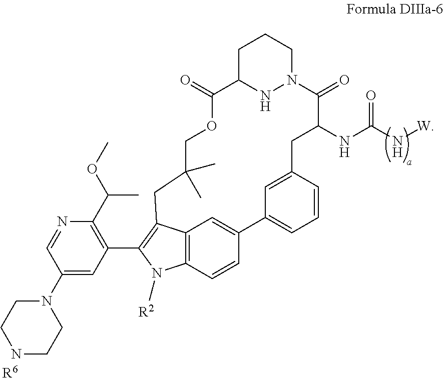

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-7:

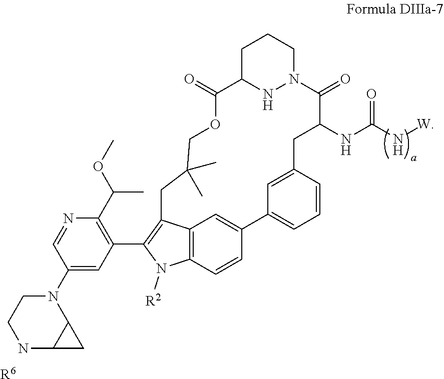

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIIa-6 or DIIIa-7), R⁶ is methyl.

In some embodiments, the RAS(ON) inhibitor, or a pharmaceutically acceptable salt thereof, has the structure of Formula DIIIa-8 or Formula DIIIa-9:

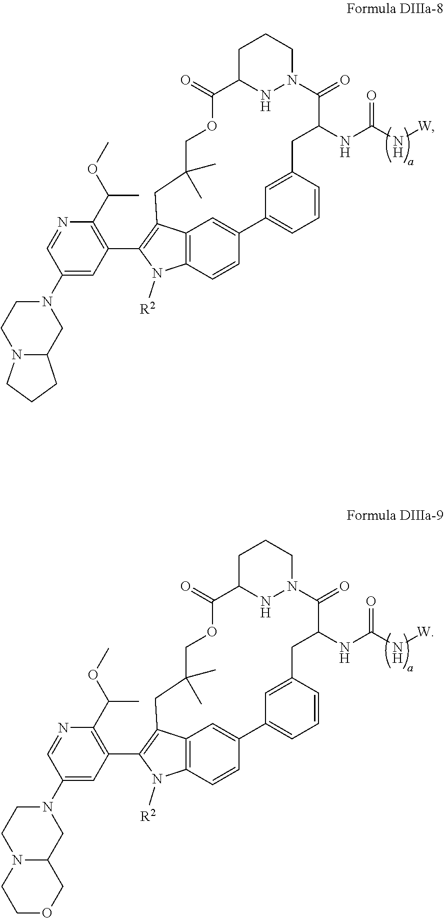

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS(ON) inhibitor, or a pharmaceutically acceptable salt thereof, has the structure of Formula DIVa:

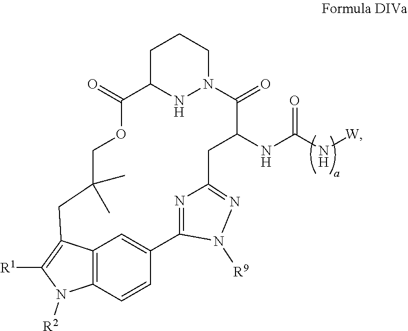

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
a is 0 or 1.

In some embodiments, the RAS(ON) inhibitor, or a pharmaceutically acceptable salt thereof, has the structure of Formula DIVa-1:

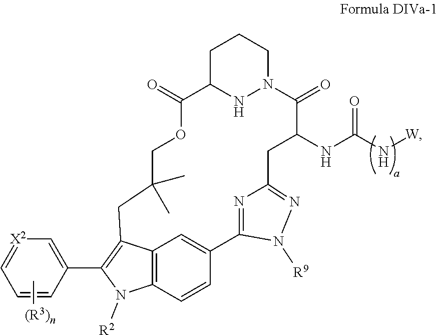

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS(ON) inhibitor, or a pharmaceutically acceptable salt thereof, has the structure of Formula DIVa-2:

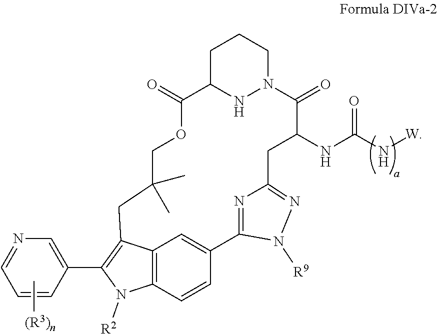

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS(ON) inhibitor, or a pharmaceutically acceptable salt thereof, has the structure of Formula DIVa-3:

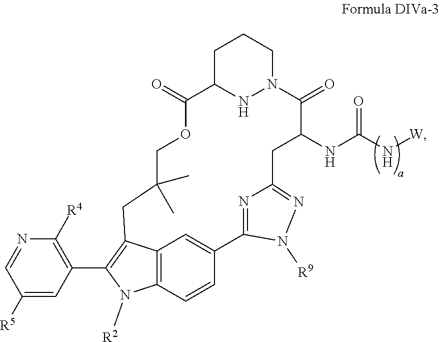

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIVa-4:

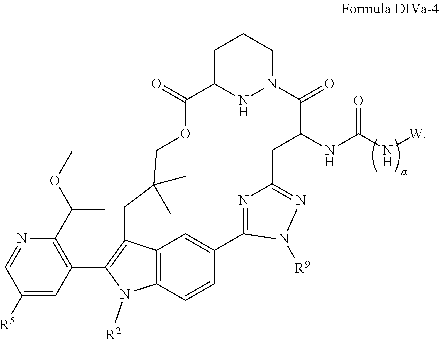

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIVa-5:

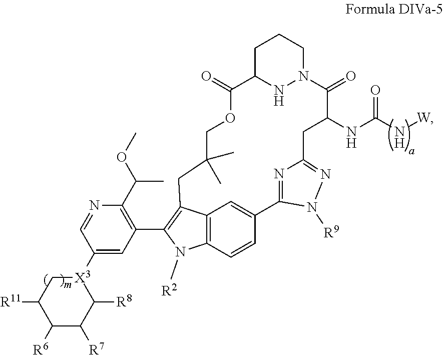

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X³ is N or CH;
m is 1 or 2;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl;
R⁶, R⁷, R⁸, and R¹¹ are each independently selected from hydrogen, optionally substituted C1-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIVa-6:

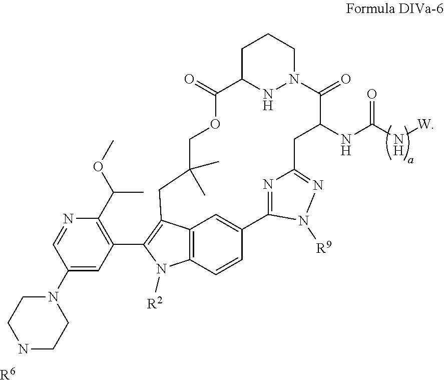

In some embodiments, the RAS(ON) inhibitor, or a pharmaceutically acceptable salt thereof, has the structure of Formula DIVa-7:

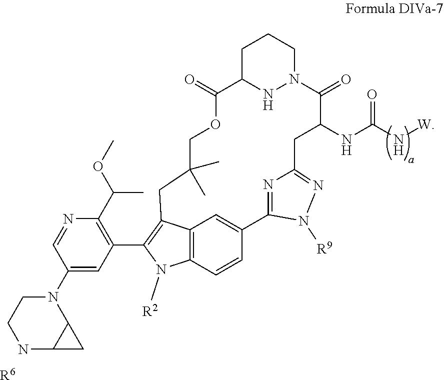

In some embodiments (e.g., of any one of Formulae DIVa-6 or DIVa-7), R⁶ is methyl.

In some embodiments, the RAS(ON) inhibitor, or a pharmaceutically acceptable salt thereof, has the structure of Formula DIVa-8 or Formula DIVa-9:

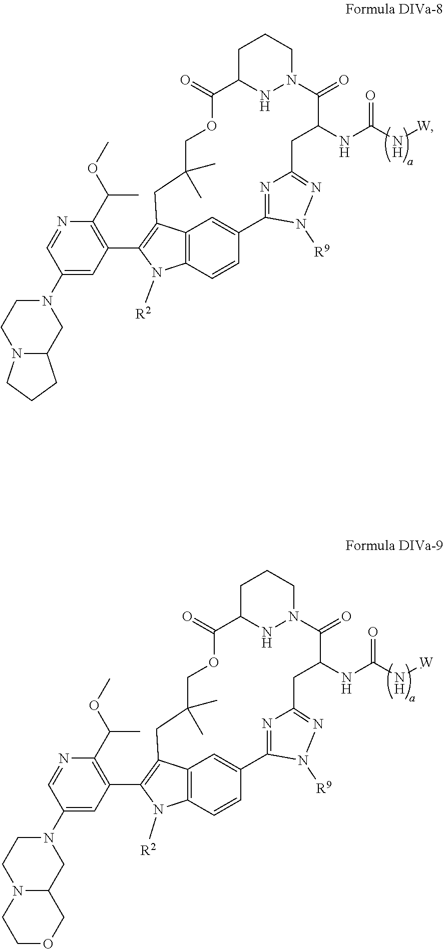

In some embodiments (e.g., of any one of Formulae DIVa, DIVa-1, DIVa-2, DIVa-3, DIVa-4, DIVa-5, DIVa-6, DIVa-7, DIVa-8, or DIVa-9), R⁹ is methyl.

In some embodiments, Y is —NHS(O)₂— or —NHS(O)₂NH—.

In some embodiments, the RAS(ON) inhibitor, or a pharmaceutically acceptable salt thereof, has the structure of Formula DVa:

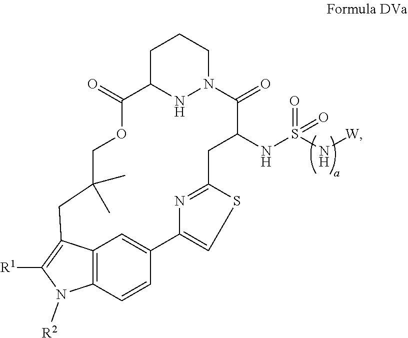

wherein a is 0 or 1.

In some embodiments, the RAS(ON) inhibitor, or a pharmaceutically acceptable salt thereof, has the structure of Formula DVa-1:

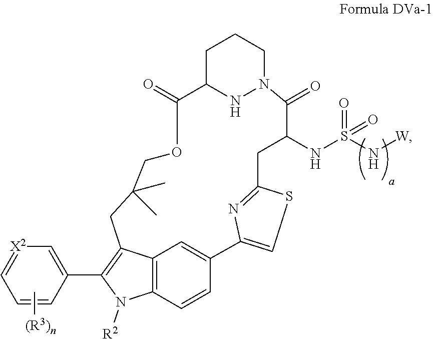

wherein X² is N or CH;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVa-2:

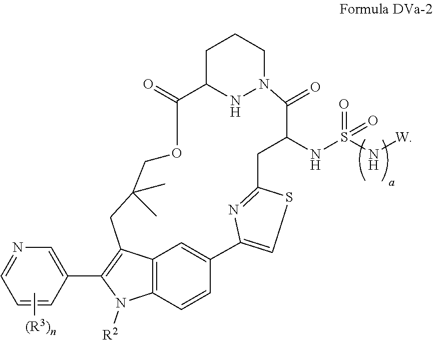

In some embodiments, the RAS(ON) inhibitor, or a pharmaceutically acceptable salt thereof, has the structure of Formula DVa-3:

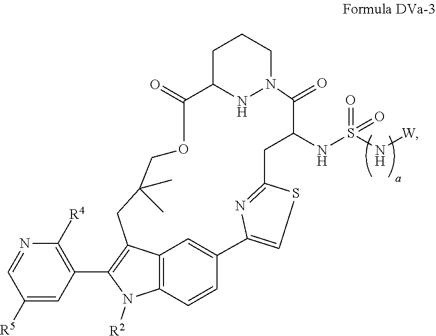

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVa-4:

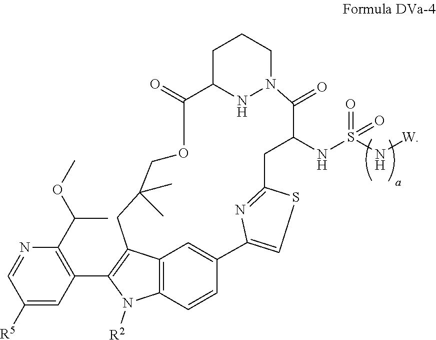

In some embodiments, the RAS(ON) inhibitor, or a pharmaceutically acceptable salt thereof, has the structure of Formula DVa-5:

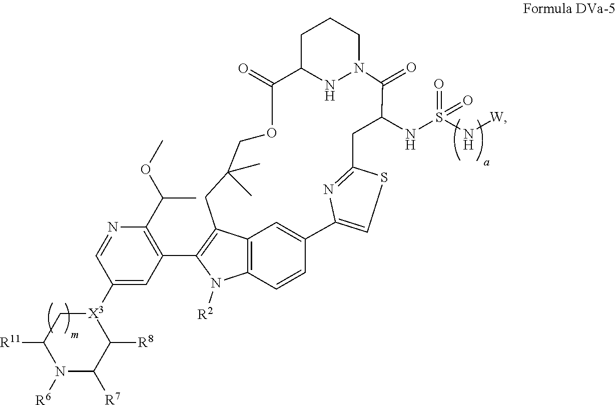

wherein X³ is N or CH;
m is 1 or 2;
R⁶, R⁷, R⁸, and R¹¹ are each independently selected from hydrogen, optionally substituted C1-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DVIa:

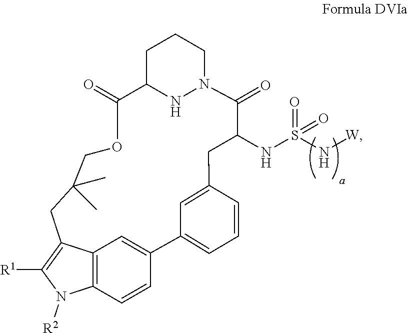

wherein a is 0 or 1.

In some embodiments, the RAS(ON) inhibitor, or a pharmaceutically acceptable salt thereof, has the structure of Formula DVIa-1:

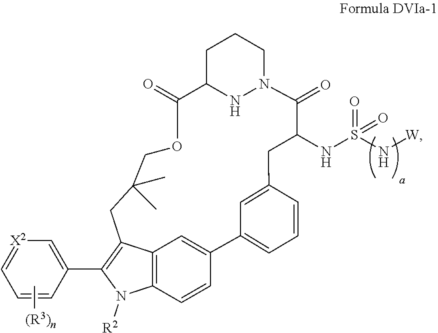

wherein X² is N or CH;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIa-2:

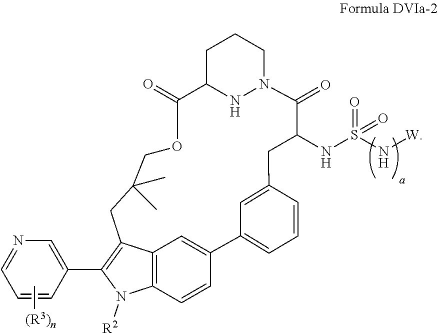

In some embodiments, the RAS(ON) inhibitor, or a pharmaceutically acceptable salt thereof, has the structure of Formula DVIa-3:

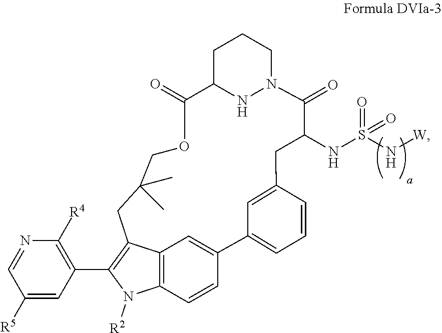

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIa-4:

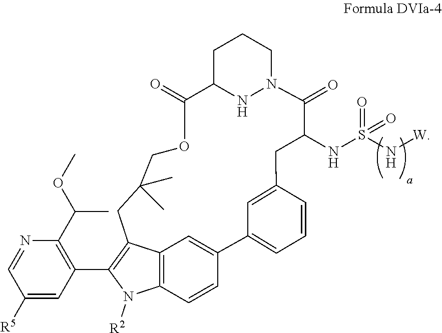

In some embodiments, the RAS(ON) inhibitor, or a pharmaceutically acceptable salt thereof, has the structure of Formula DVIa-5:

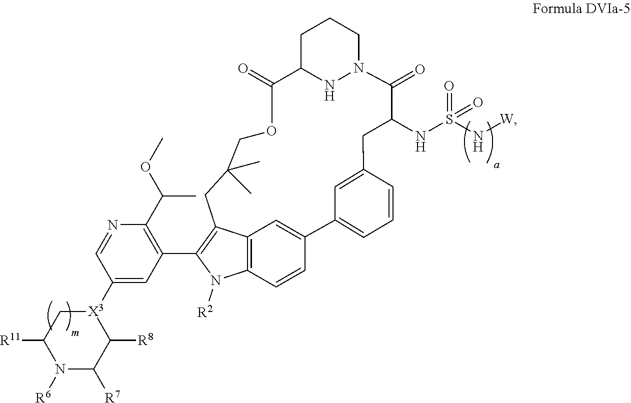

wherein X³ is N or CH;
m is 1 or 2;
R⁶, R⁷, R⁸, and R¹¹ are each independently selected from hydrogen, optionally substituted C1-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DVIIa:

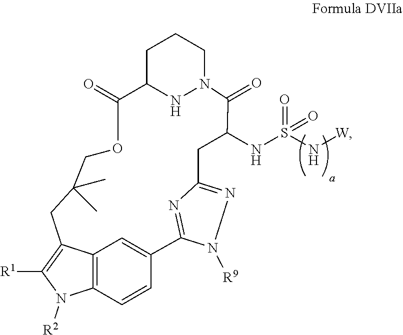

wherein R⁹ is H or C₁-C₆ alkyl; and
a is 0 or 1.

In some embodiments, the RAS(ON) inhibitor, or a pharmaceutically acceptable salt thereof, has the structure of Formula DVIIa-1:

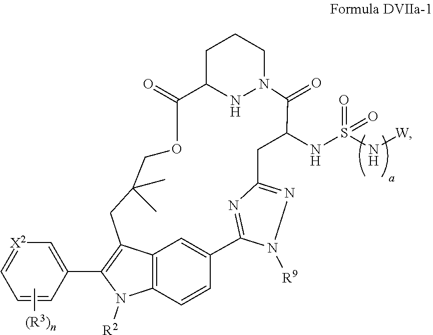

wherein X² is N or CH;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIIa-2:

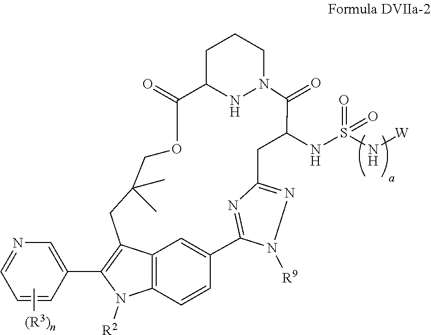

In some embodiments, the RAS(ON) inhibitor, or a pharmaceutically acceptable salt thereof, has the structure of Formula DVIIa-3:

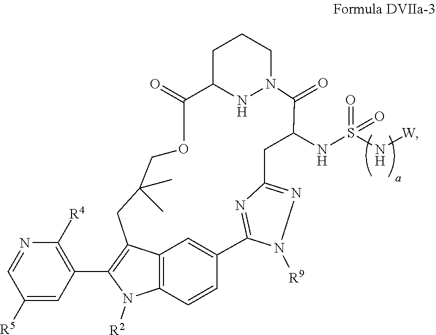

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIIa-4:

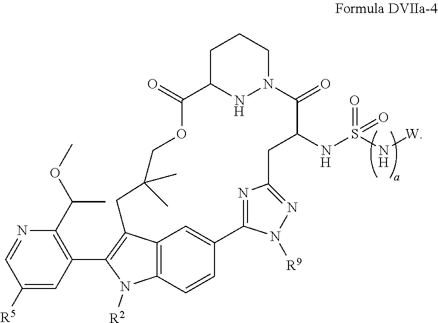

In some embodiments, the RAS(ON) inhibitor, or a pharmaceutically acceptable salt thereof, has the structure of Formula DVIIa-5:

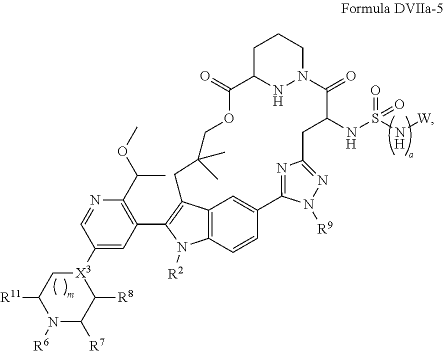

wherein X³ is N or CH;
m is 1 or 2;
R⁶, R⁷, R⁸, and R¹¹ are each independently selected from hydrogen, optionally substituted C1-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DVIIa, DVIIa-1, DVIIa-2, DVIIa-3, DVIIa-4, or DVIIa-5), R⁹ is methyl.

In some embodiments, Y is —NHS(O)— or —NHS(O)NH—.

In some embodiments, the RAS(ON) inhibitor, or a pharmaceutically acceptable salt thereof, has the structure of Formula DVIIIa:

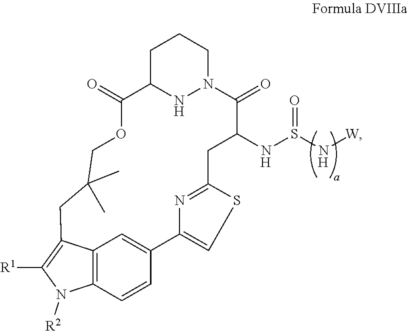

wherein a is 0 or 1.

In some embodiments, the RAS(ON) inhibitor, or a pharmaceutically acceptable salt thereof, has the structure of Formula VIIIa-1:

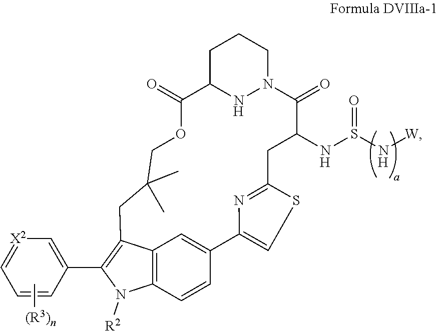

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIIIa-2:

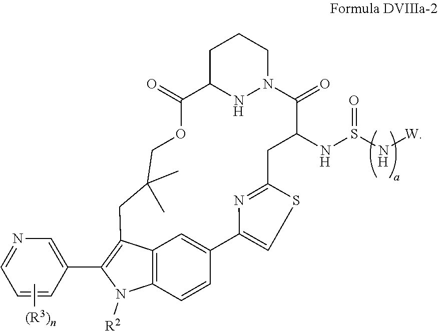

In some embodiments, the RAS(ON) inhibitor, or a pharmaceutically acceptable salt thereof, has the structure of Formula DVIIIa-3:

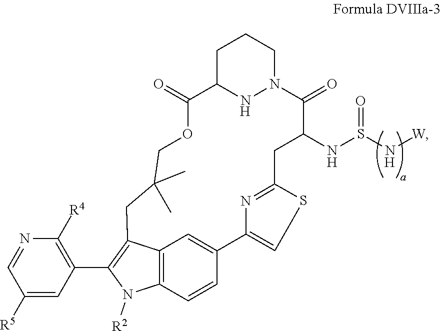

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIIIa-4:

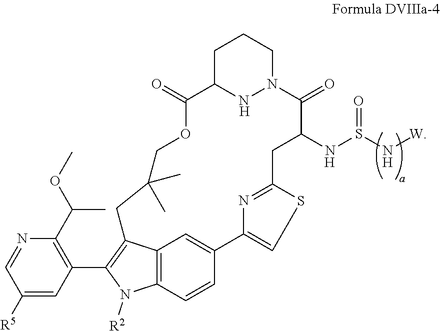

In some embodiments, the RAS(ON) inhibitor, or a pharmaceutically acceptable salt thereof, has the structure of Formula DVIIIa-5:

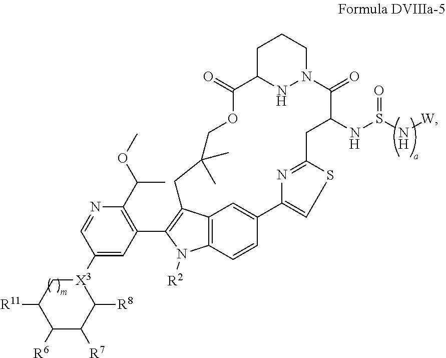

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIXa:

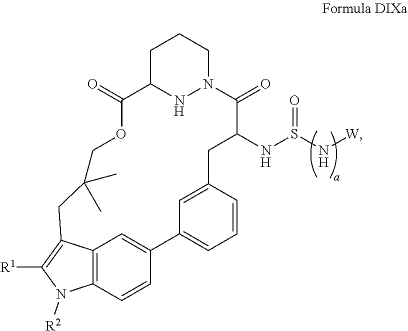

wherein a is 0 or 1.

In some embodiments, the RAS(ON) inhibitor, or a pharmaceutically acceptable salt thereof, has the structure of Formula DIXa-1:

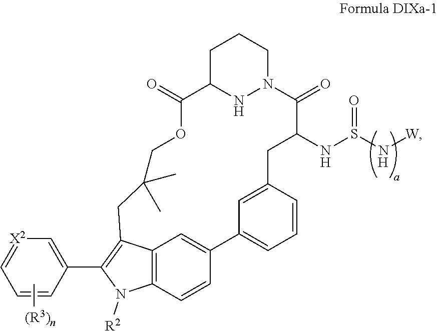

wherein X² is N or CH;
each R³ is independently selected from halogen, cyano, hydroxy, optionally substituted amine, optionally substituted amido, optionally substituted C₁-C₆ alkyl, optionally substituted C1-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIXa-2:

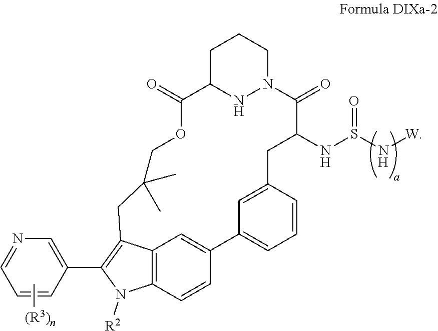

In some embodiments, the RAS(ON) inhibitor, or a pharmaceutically acceptable salt thereof, has the structure of Formula DIXa-3:

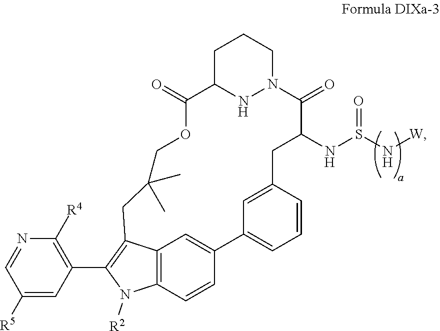

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIXa-4:

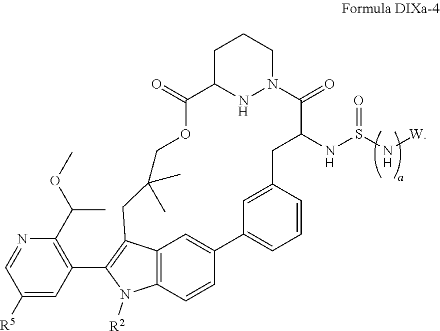

In some embodiments, the RAS(ON) inhibitor, or a pharmaceutically acceptable salt thereof, has the structure of Formula DIXa-5:

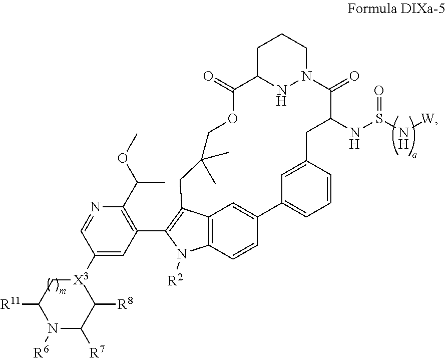

wherein X³ is N or CH;
m is 1 or 2;
R⁶, R⁷, R⁸, and R¹¹ are each independently selected from hydrogen, optionally substituted C1-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DXa:

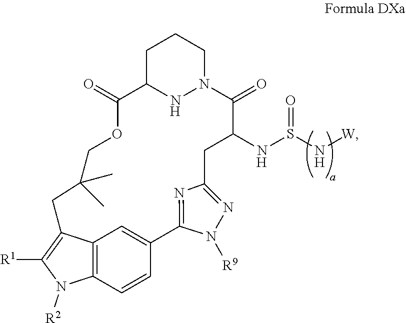

wherein R⁹ is H or C₁-C₆ alkyl; and
a is 0 or 1.

In some embodiments, the RAS(ON) inhibitor, or a pharmaceutically acceptable salt thereof, has the structure of Formula DXa-1:

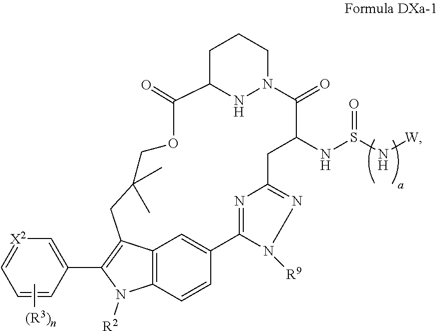

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DXa-2:

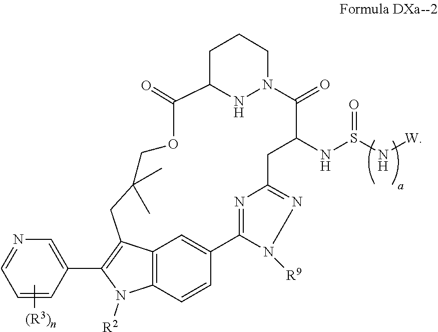

In some embodiments, the RAS(ON) inhibitor, or a pharmaceutically acceptable salt thereof, has the structure of Formula DXa-3:

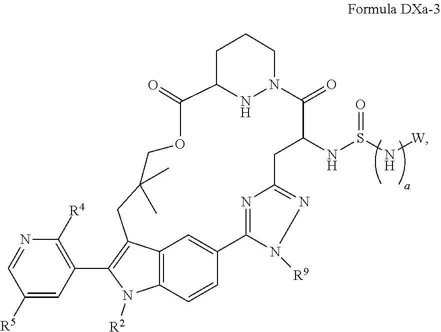

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DXa-4:

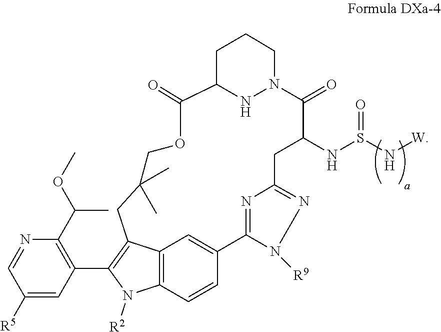

In some embodiments, the RAS(ON) inhibitor, or a pharmaceutically acceptable salt thereof, has the structure of Formula DXa-5:

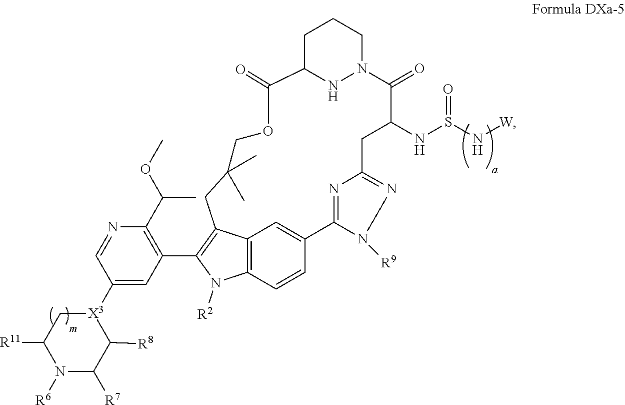

wherein X³ is N or CH;
m is 1 or 2;
R⁶, R⁷, R⁸, and R¹¹ are each independently selected from hydrogen, optionally substituted C1-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DXa, DXa-1, DXa-2, DXa-3, DXa-4, or DXa-5), R⁹ is methyl.

In some embodiments of formula DIa or subformula thereof, a is 0. In some embodiments of formula DIa or subformula thereof, a is 0.

In some embodiments of formula DIa or subformula thereof, R² is optionally substituted C₁-C₆ alkyl. In some embodiments, R² is selected from —CH₂CH₃ or —CH₂CF₃.

In some embodiments of formula DIa or subformula thereof, W is C₁-C₄ alkyl. In some embodiments, W is:

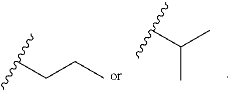

In some embodiments of formula DIa or subformula thereof, W is optionally substituted cyclopropyl, optionally substituted cyclobutyl, optionally substituted cyclopentyl, or optionally substituted cyclohexyl, optionally substituted piperidine, optionally substituted piperazine, optionally substituted pyridine, or optionally substituted phenyl.

In some embodiments of formula DIa or subformula thereof, W is optionally substituted 3 to 10-membered heterocycloalkyl, optionally substituted 3 to 10-membered cycloalkyl, optionally substituted 6 to 10-membered aryl, or optionally substituted 5 to 10-membered heteroaryl.

In some embodiments of formula DIa or subformula thereof, W is optionally substituted 3 to 10-membered heterocycloalkyl.

In some embodiments, W is selected from the following, or a stereoisomer thereof:

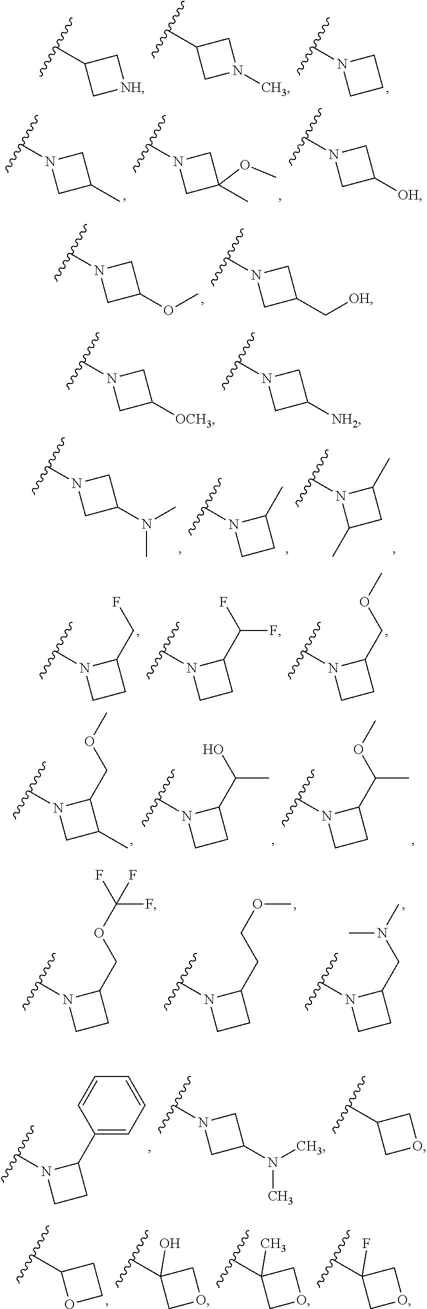

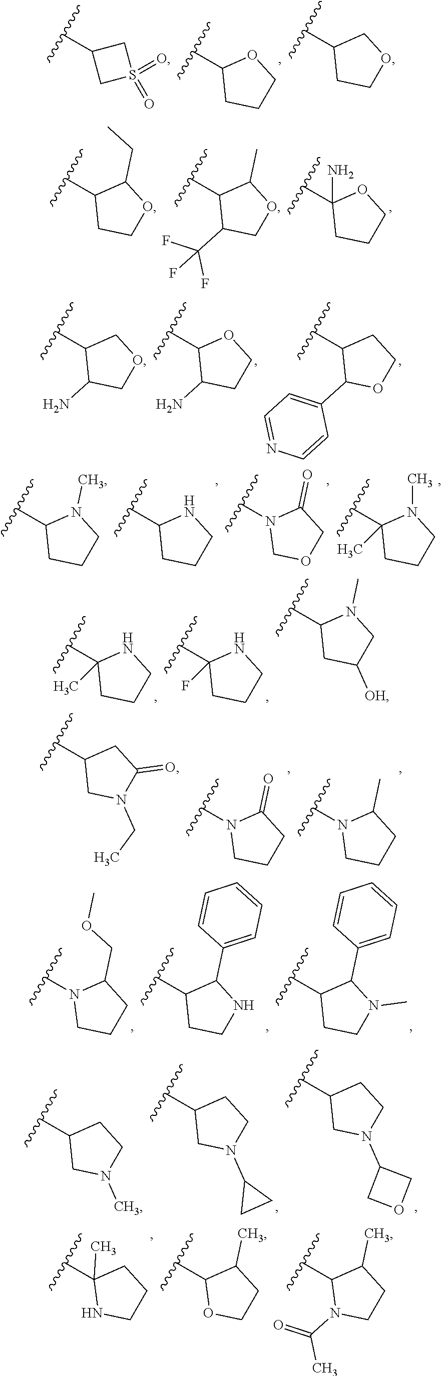

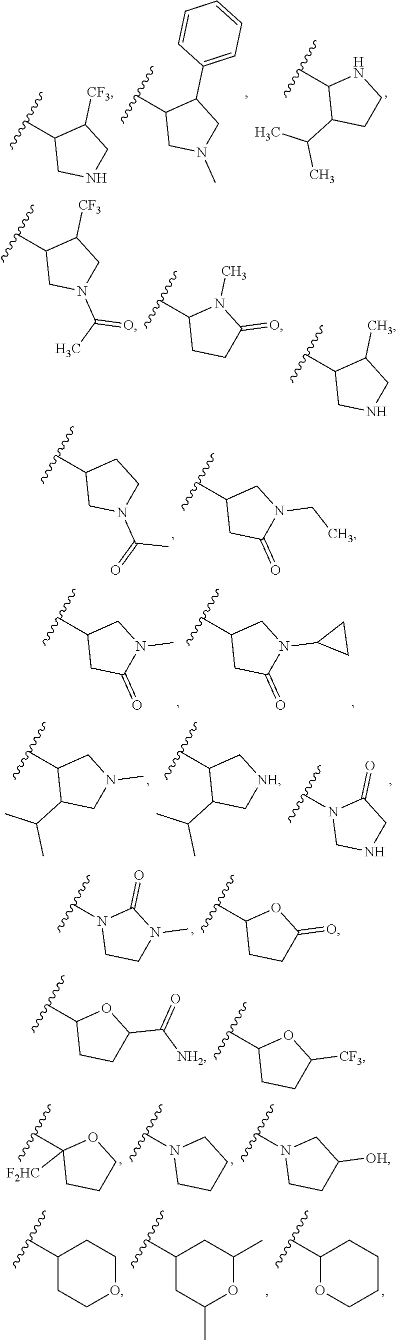

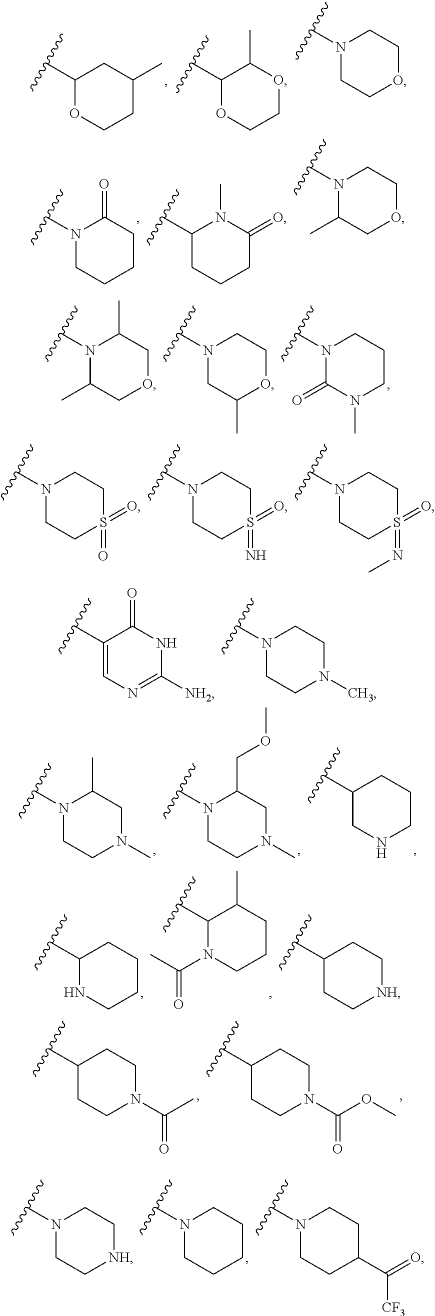

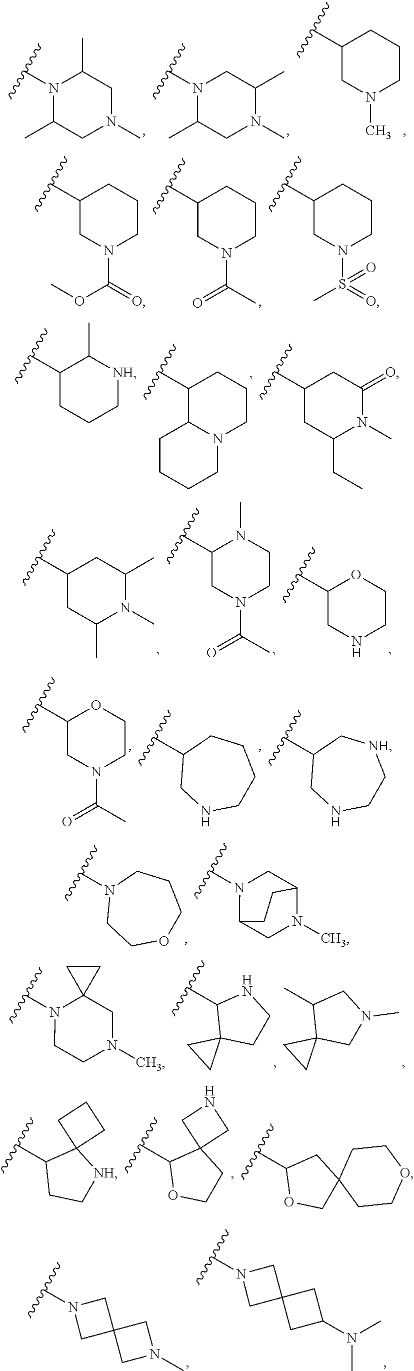

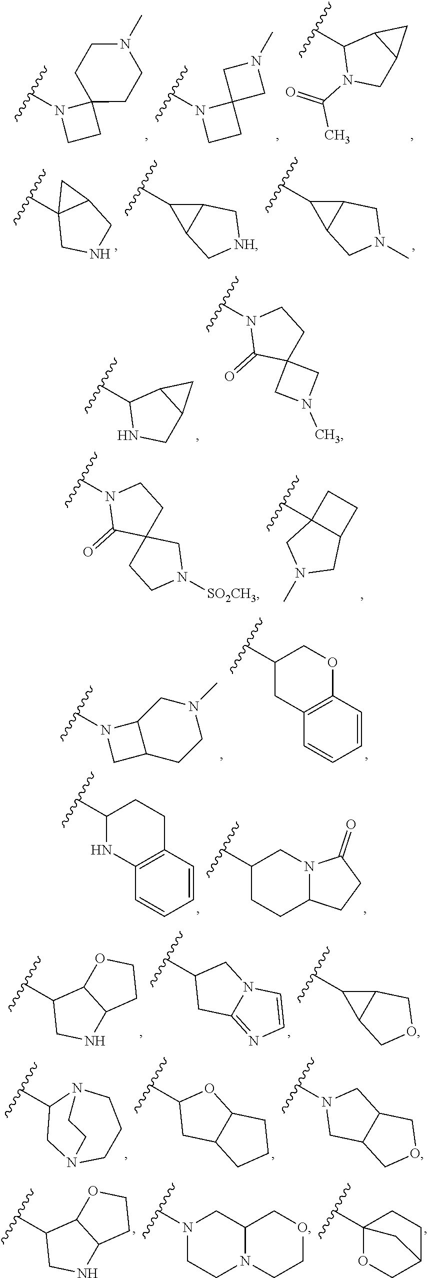

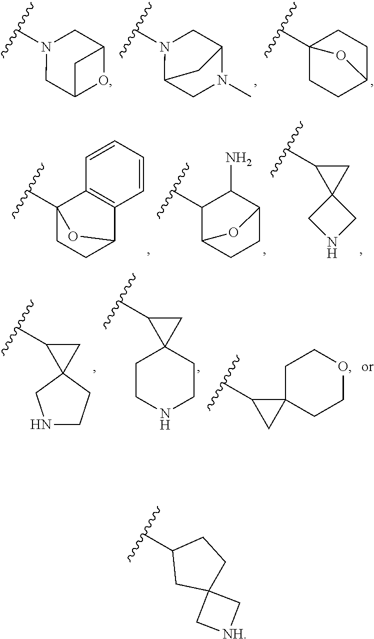

In some embodiments, W is selected from the following, or a stereoisomer thereof:

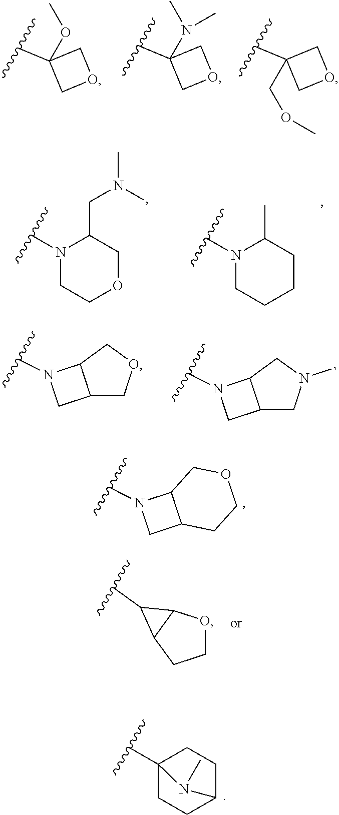

In some embodiments of formula DIa or subformula thereof, W is optionally substituted 3 to 10-membered cycloalkyl.

In some embodiments, W is selected from the following, or a stereoisomer thereof:

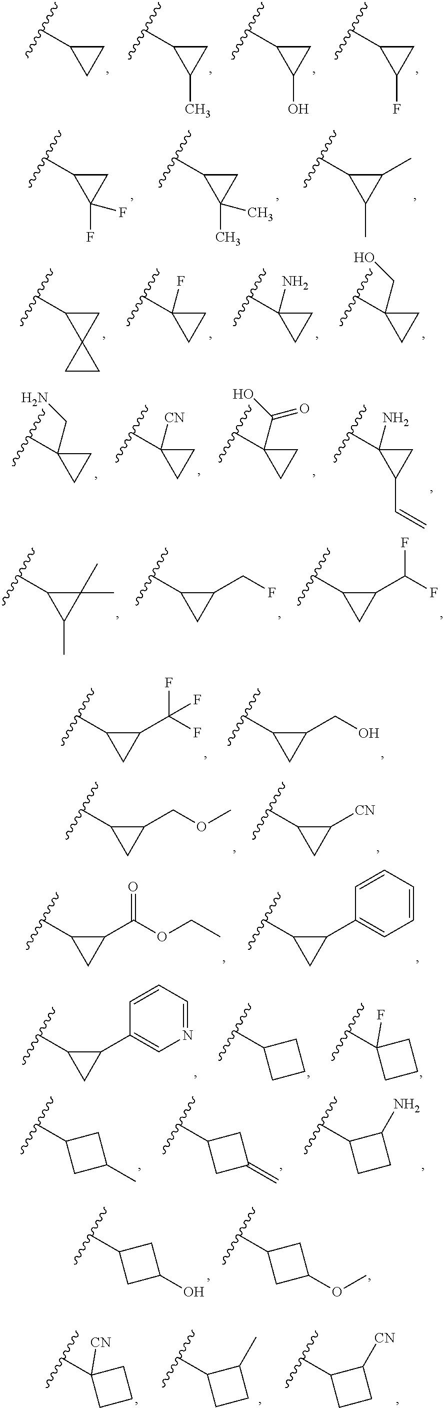

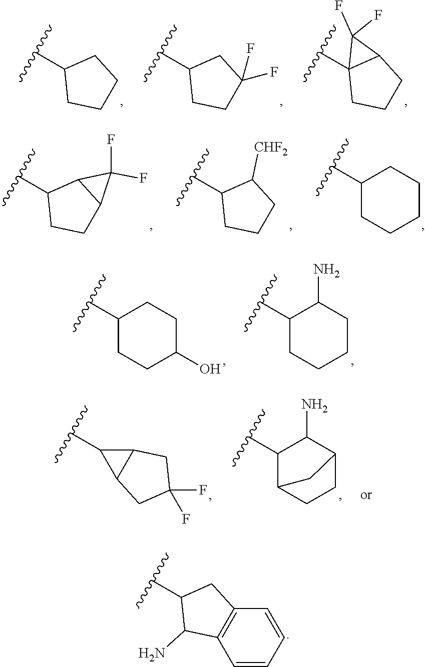

In some embodiments, W is selected from the following, or a stereoisomer thereof:

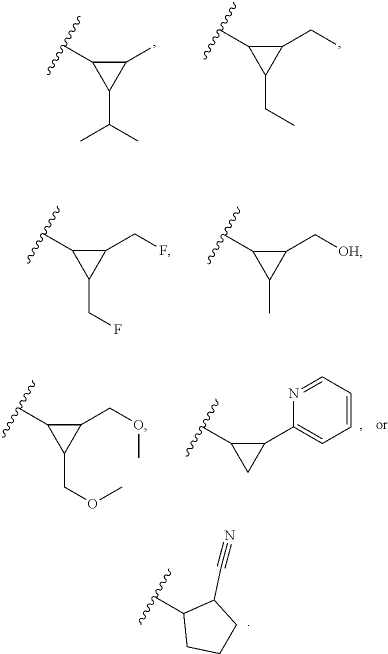

In some embodiments of formula DIa or subformula thereof, W is optionally substituted 5 to 10-membered heteroaryl.

In some embodiments, W is selected from the following, or a stereoisomer thereof:

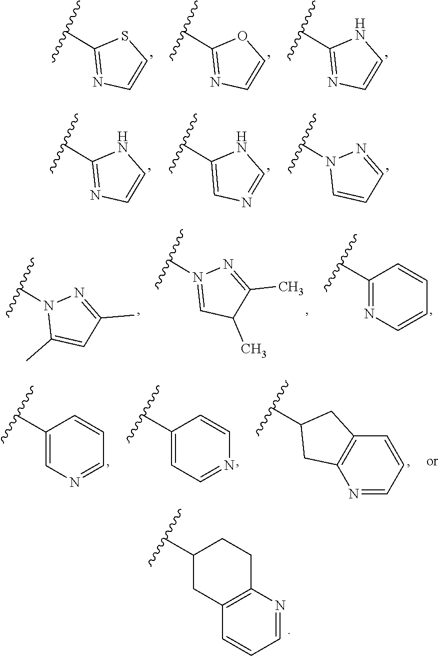

In some embodiments of formula DIa or subformula thereof, W is optionally substituted 6 to 10-membered aryl. In some embodiments, W is optionally substituted phenyl.

In some embodiments of formula DIa or subformula thereof, W is optionally substituted C₁-C₃ heteroalkyl. In some embodiments, W is selected from the following, or a stereoisomer thereof:

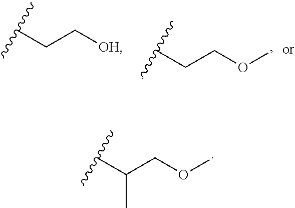

In some embodiments, the RAS(ON) inhibitor, or pharmaceutically acceptable salt thereof, has the structure of Formula DIb:

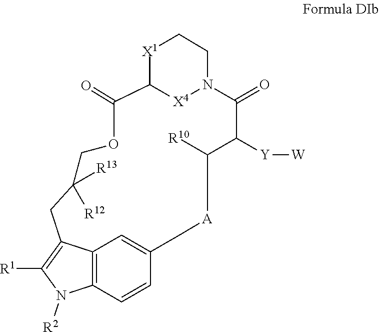

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is

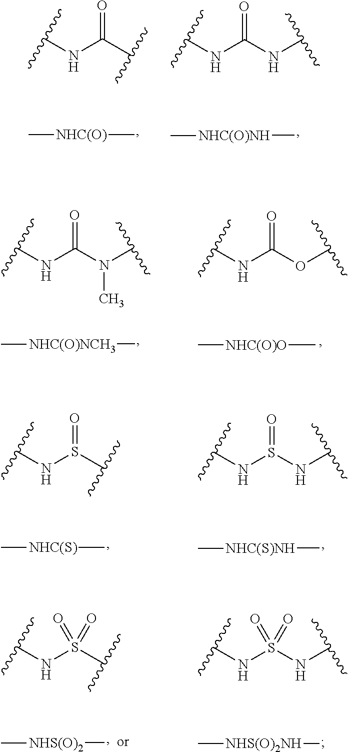

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; or W is —R¹⁴C(═O)R¹⁵ where R¹⁴ is 3 to 10-membered cycloalkylene and R¹⁵ is selected from optionally substituted 3 to 10-membered cycloalkyl, optionally substituted 6 to 10-membered aryl, or optionally substituted 5 to 10-membered heteroaryl;
X¹ and X⁴ are each, independently, CH₂, CH(CH₃) or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3 to 7-membered heterocycloalkyl; and
R¹² and R¹³ are each, independently, selected from F or CH₃, or R¹² and R¹³ combine with the atoms to which they are attached to make a 3-membered cycloalkyl

In some embodiments, the RAS(ON) inhibitor is selected from Table D1a, or a pharmaceutically acceptable salt or stereoisomer thereof. In some embodiments, the RAS(ON) inhibitor is selected from Table D1a, or a pharmaceutically acceptable salt or atropisomer thereof.

TABLE D1a
Certain Compounds of the Present Invention

| Ex. # | Structure |
|---|---|
| DA1 | 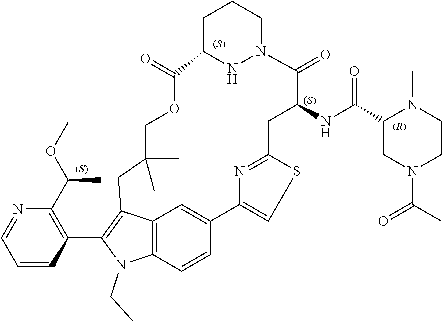 |
| DA2 | 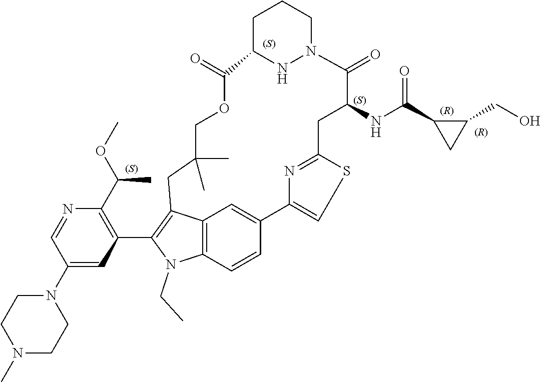 |
| DA3 | 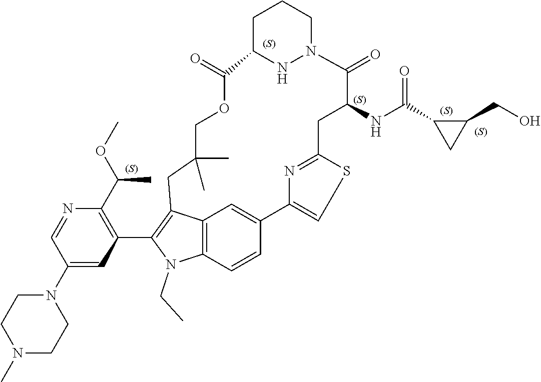 |
| DA4 | 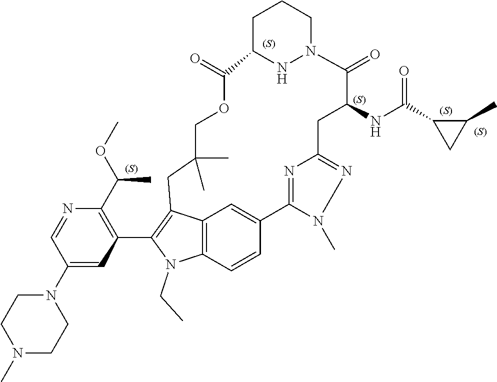 |
| DA5 | 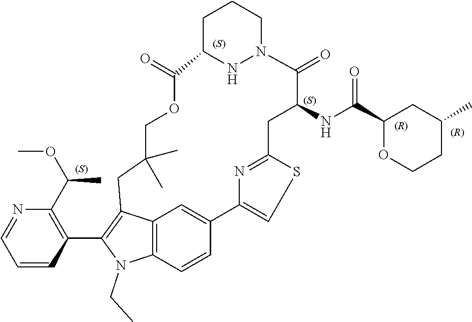 |
| DA6 | 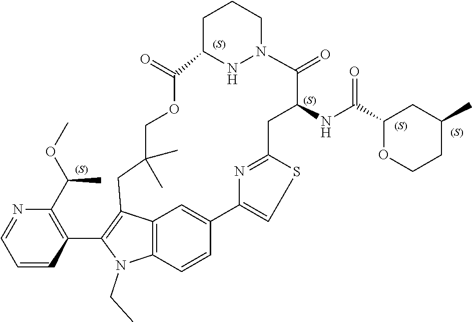 |
| DA7 | 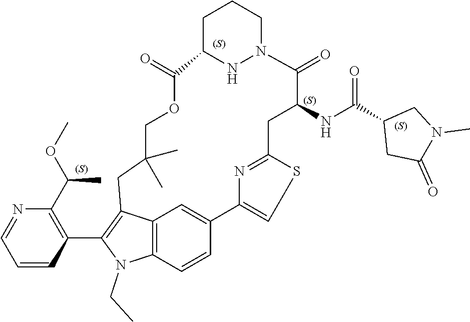 |
| DA8 | 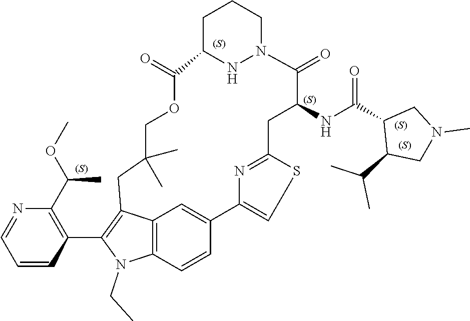 |
| DA9 | 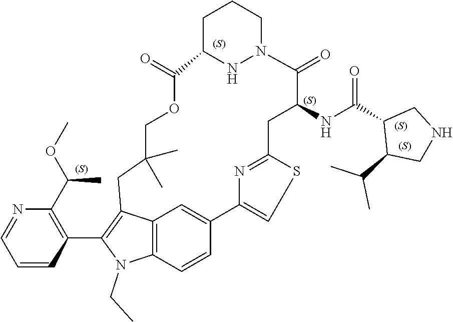 |
| DA10 | 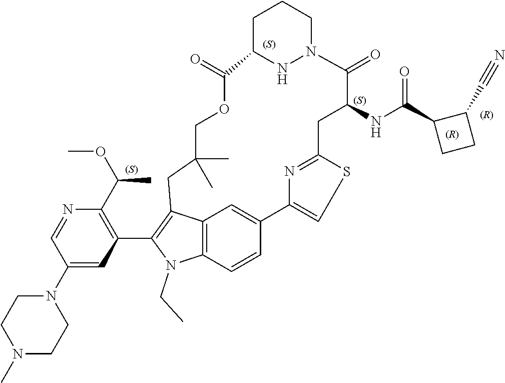 |
| DA11 | 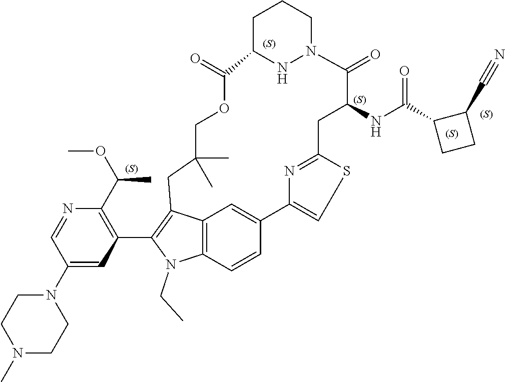 |
| DA12 | 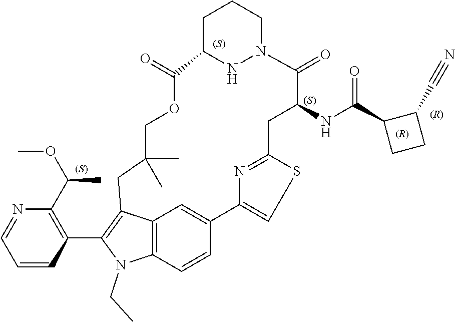 |
| DA13 | 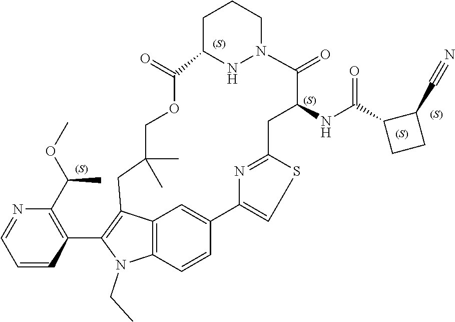 |
| DA14 | 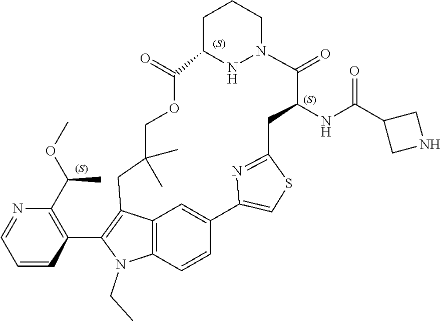 |
| DA15 | 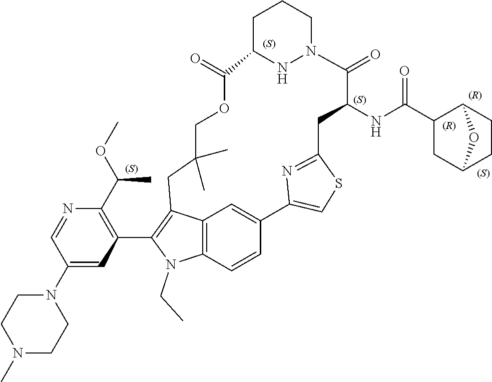 |
| DA16 | 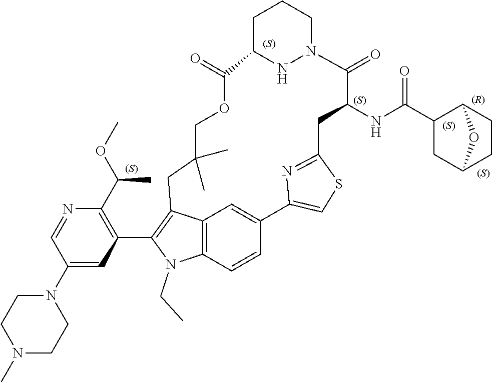 |
| DA17 | 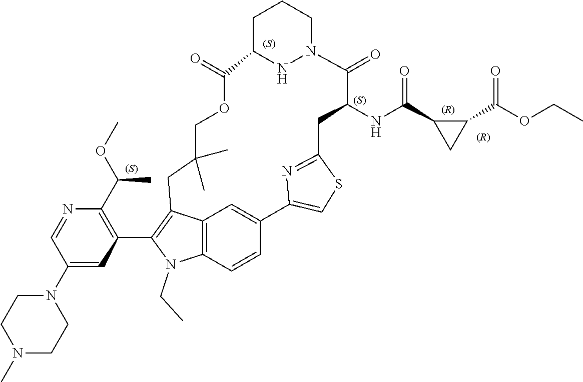 |
| DA18 | 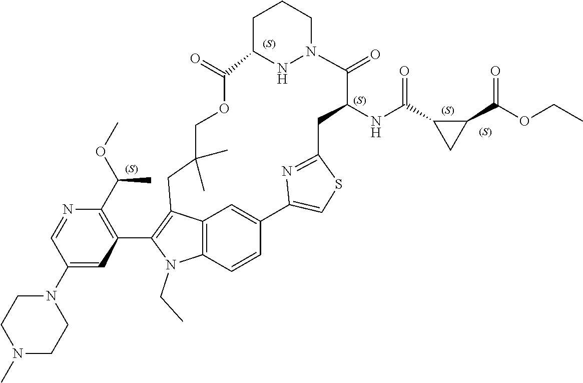 |
| DA19 | 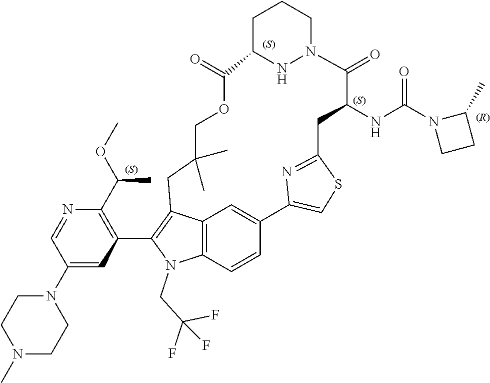 |
| DA20 | 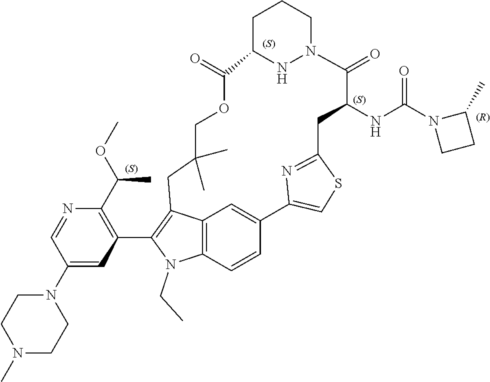 |
| DA21 | 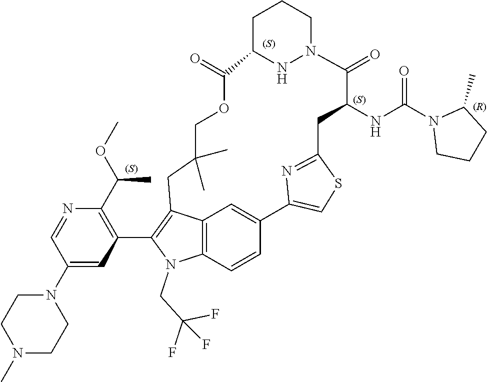 |
| DA22 | 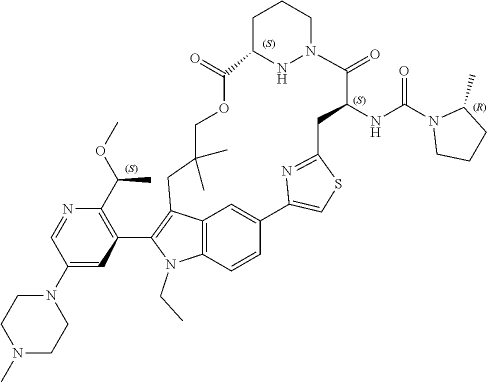 |
| DA23 | 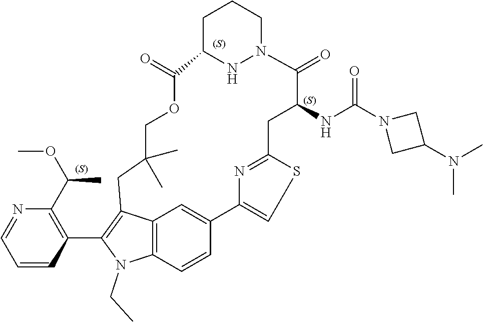 |
| DA24 | 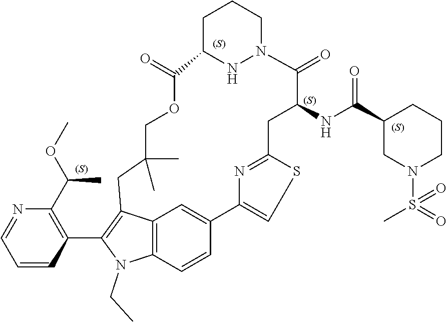 |
| DA25 | 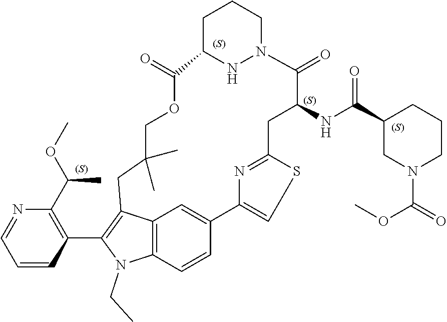 |
| DA26 | 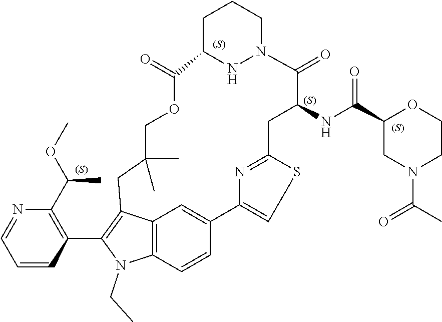 |
| DA27 | 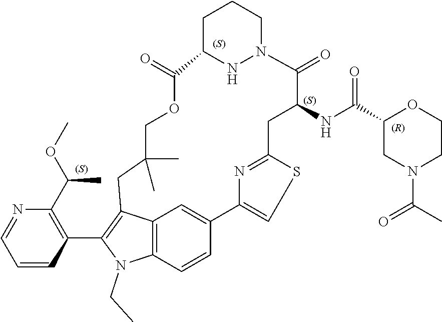 |
| DA28 | 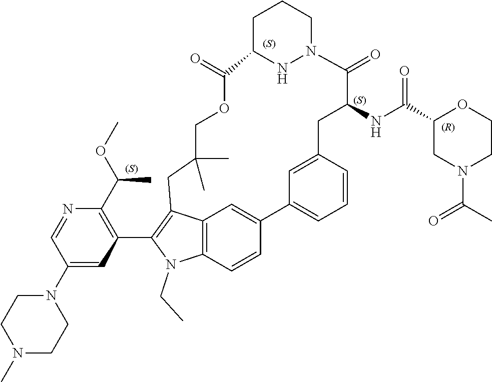 |
| DA29 | 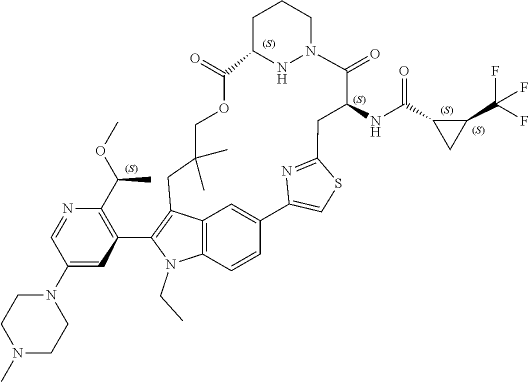 |
| DA30 | 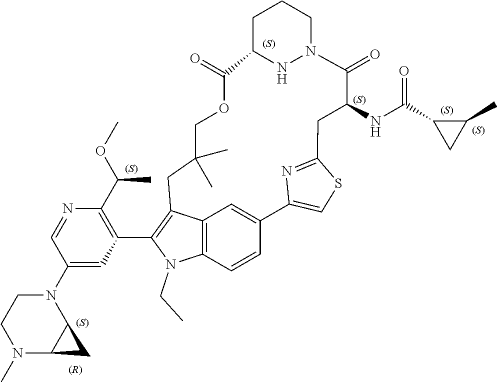 |
| DA31 | 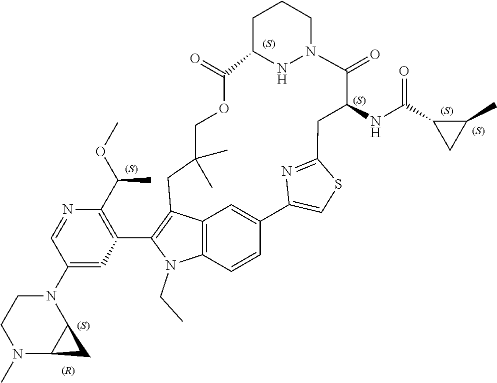 |
| DA32 | 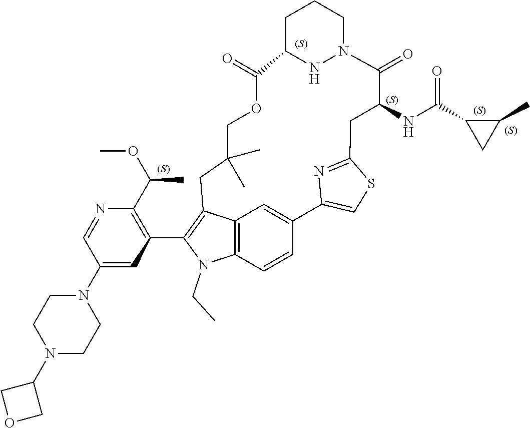 |
| DA33 | 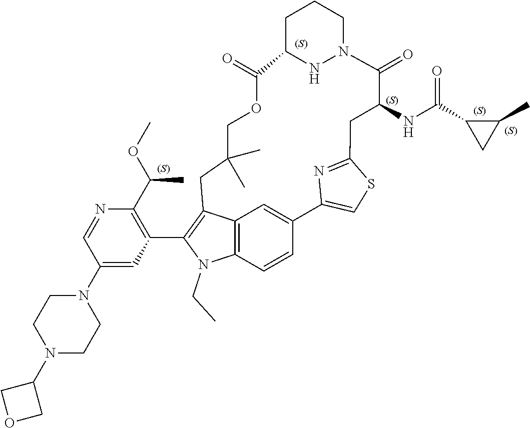 |
| DA34 | 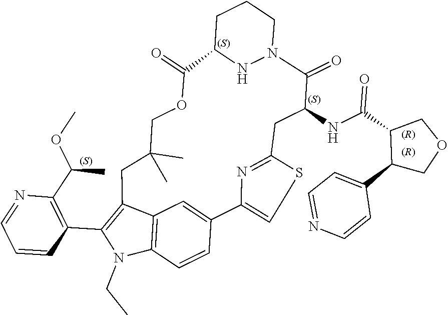 |
| DA35 | 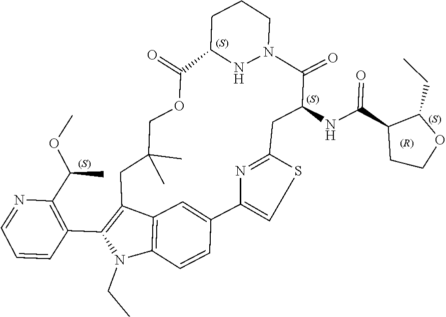 |
| DA36 | 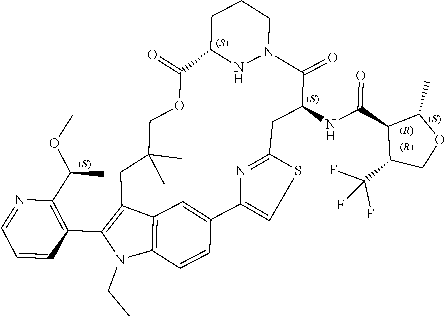 |
| DA37 | 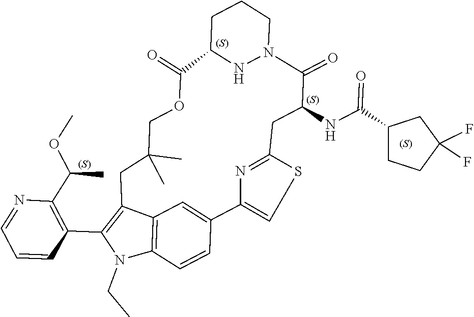 |
| DA38 | 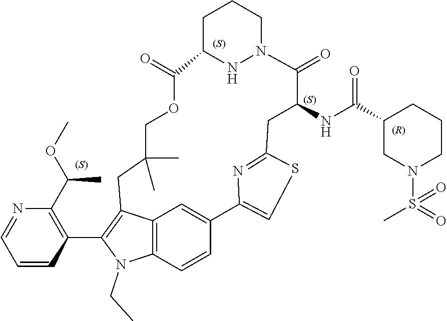 |
| DA39 | 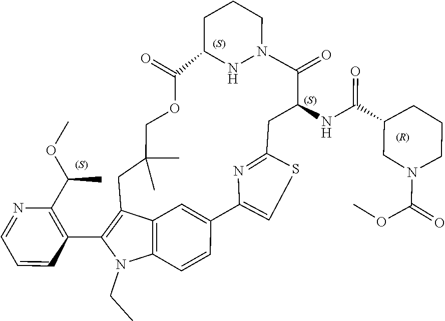 |
| DA40 | 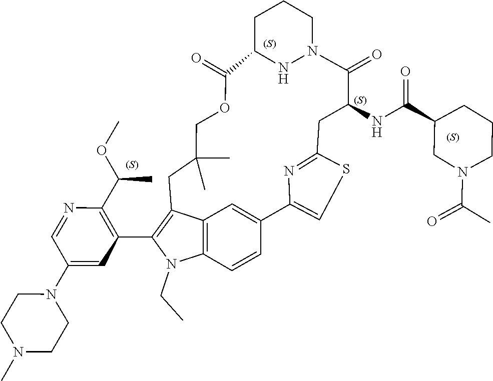 |
| DA41 | 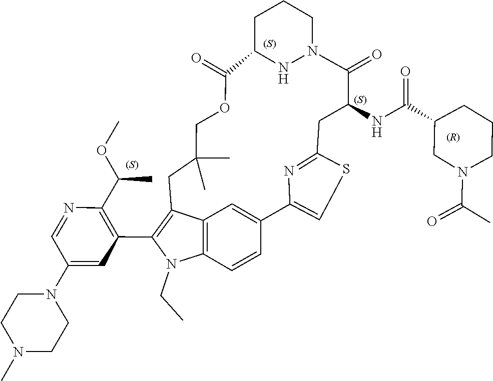 |
| DA42 | 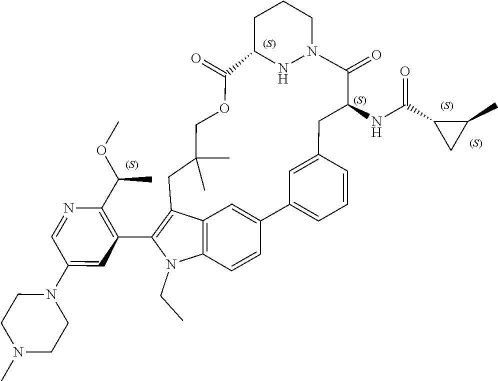 |
| DA43 | 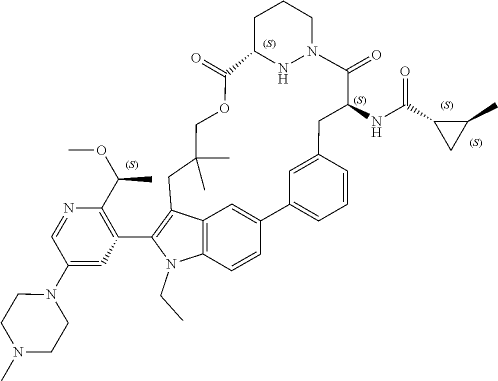 |
| DA44 | 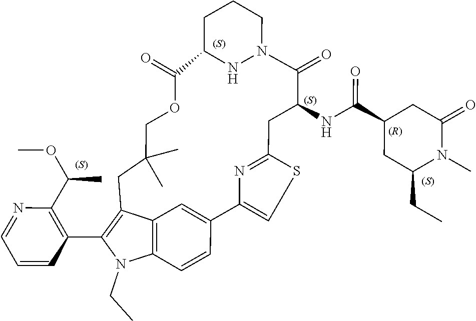 |
| DA45 | 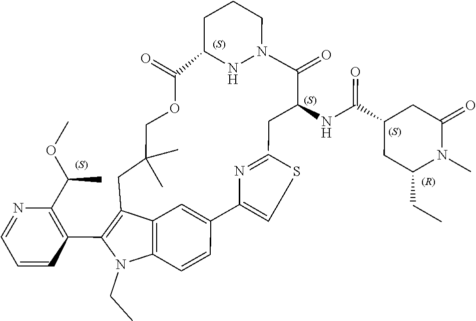 |
| DA46 | 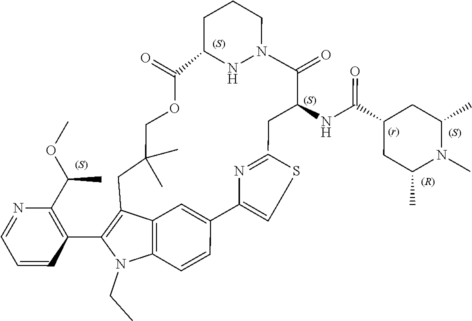 |
| DA47 | 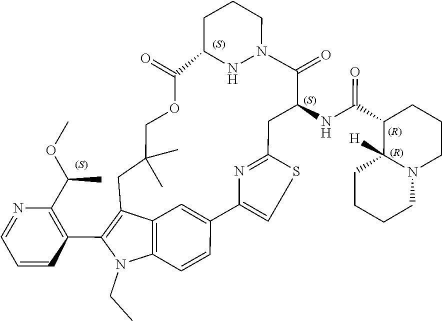 |
| DA48 | 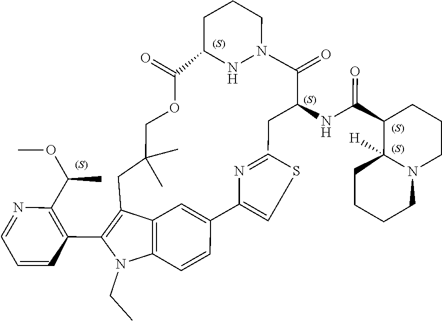 |
| DA49 | 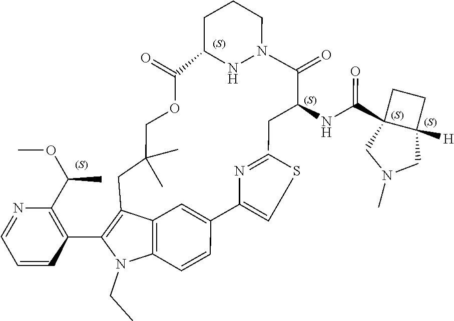 |
| DA50 | 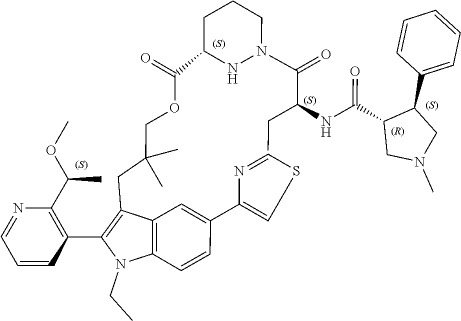 |
| DA51 | 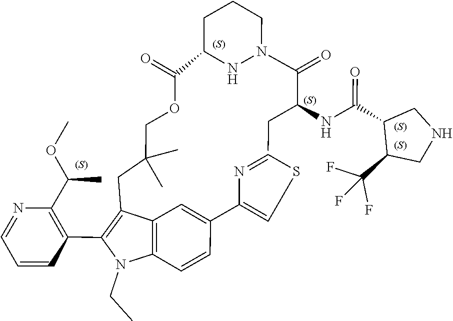 |
| DA52 | 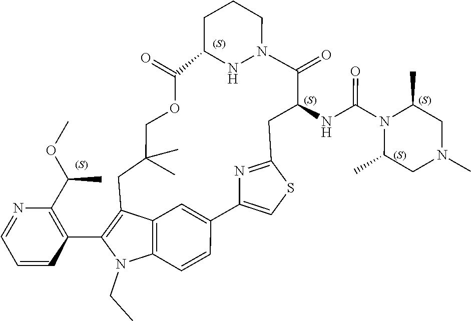 |
| DA53 | 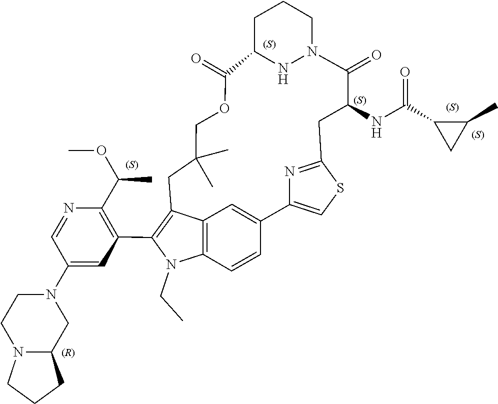 |
| DA54 | 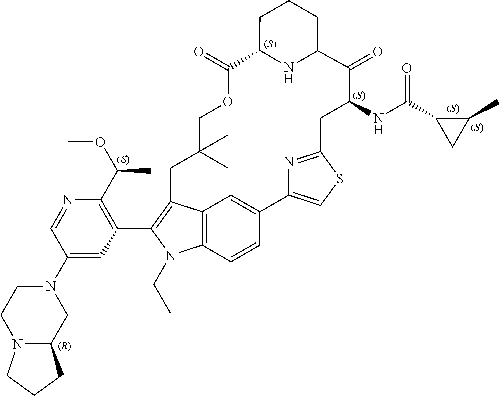 |
| DA55 | 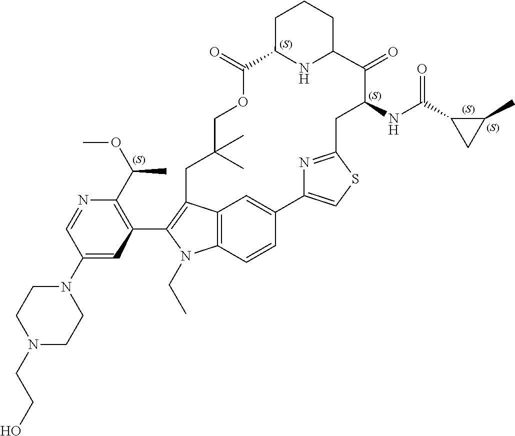 |
| DA56 | 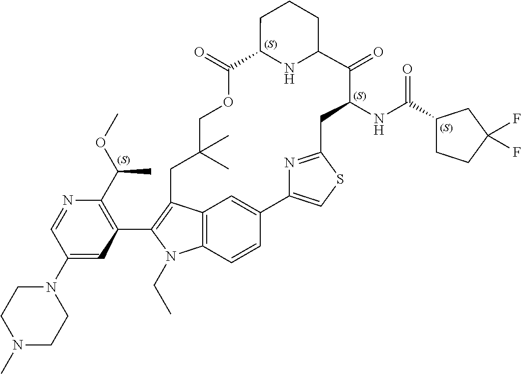 |
| DA57 | 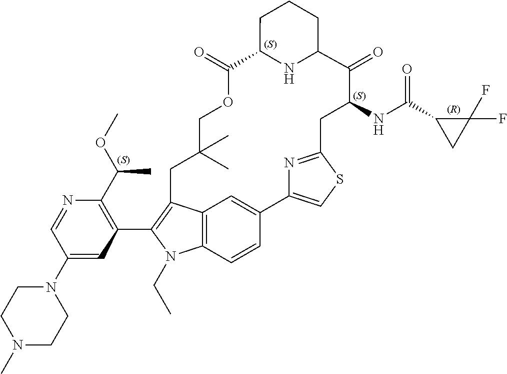 |
| DA58 | 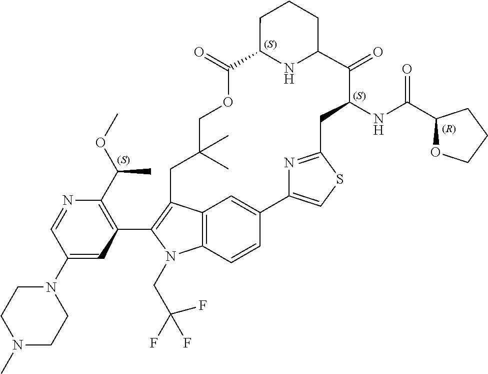 |
| DA59 | 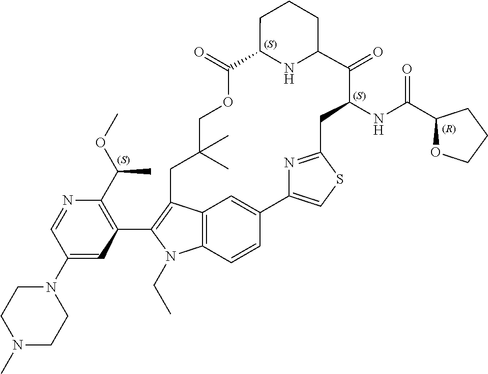 |
| DA60 | 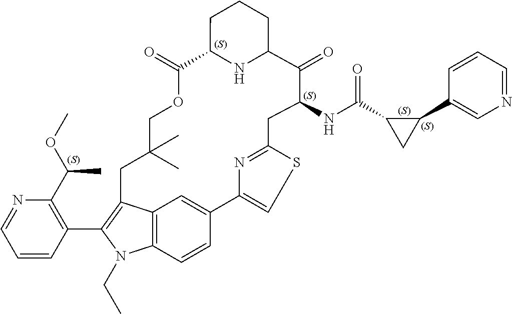 |
| DA61 | 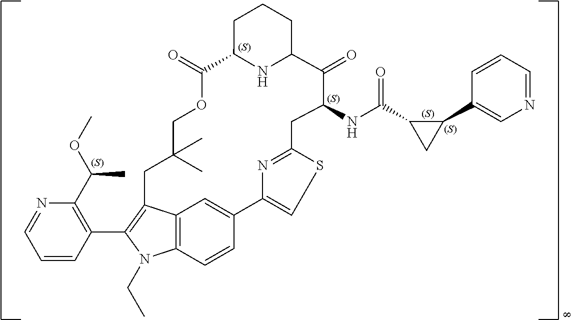 |
| DA62 | 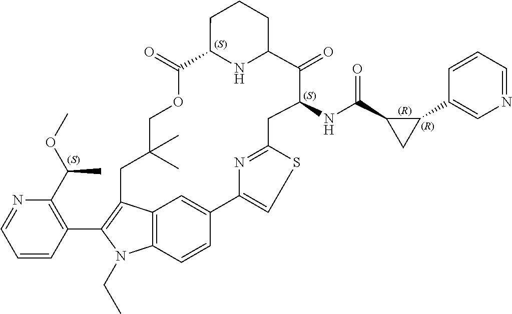 |
| DA63 | 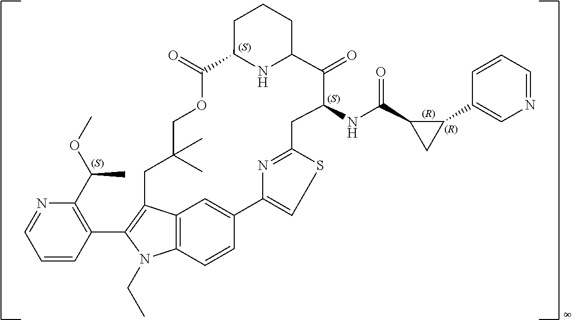 |
| DA64 | 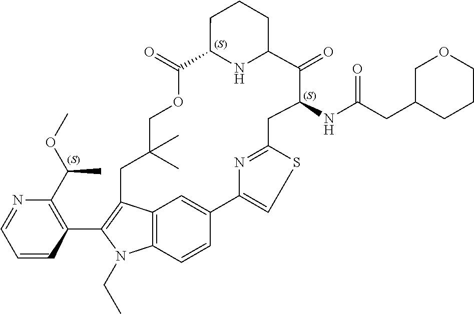 |
| DA65 | 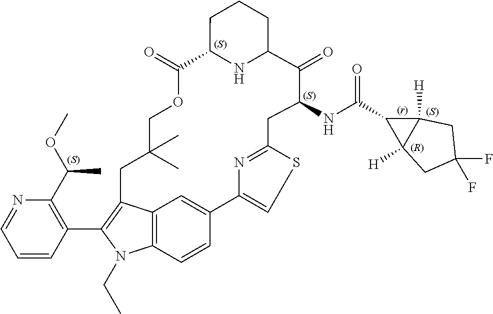 |
| DA66 | 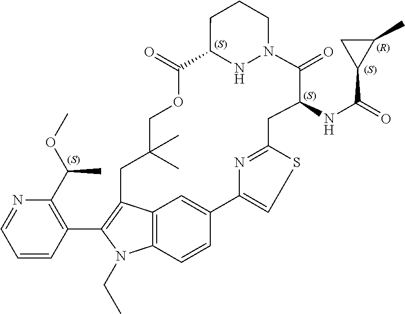 |
| DA67 | 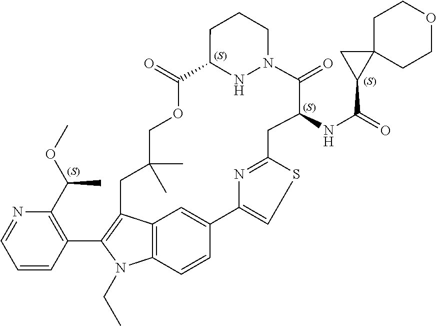 |
| DA68 | 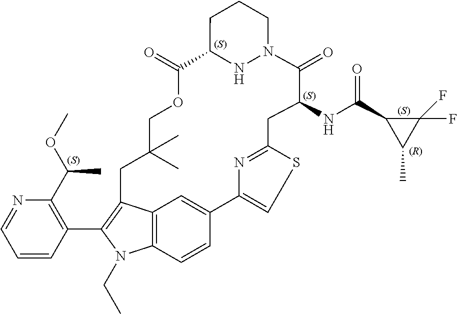 |
| DA69 | 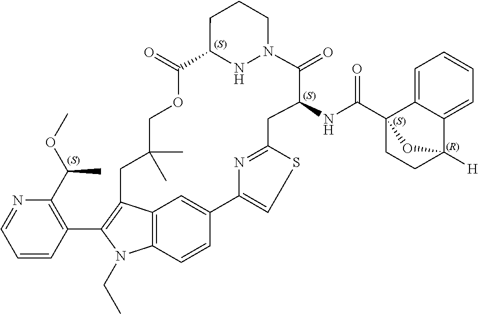 |
| DA70 | 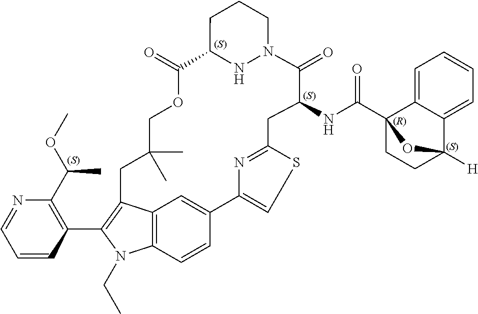 |
| DA71 | 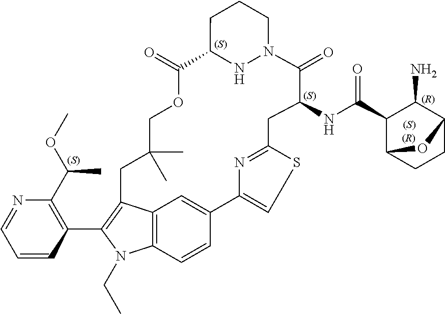 |
| DA72 | 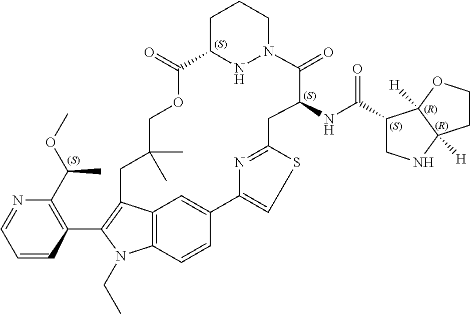 |
| DA73 | 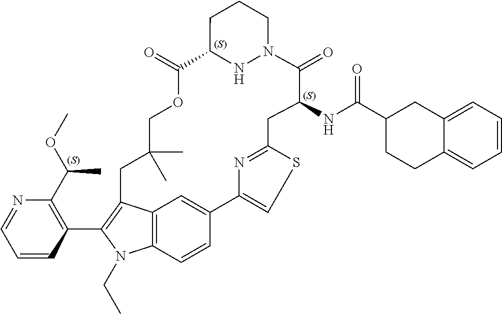 |
| DA74 | 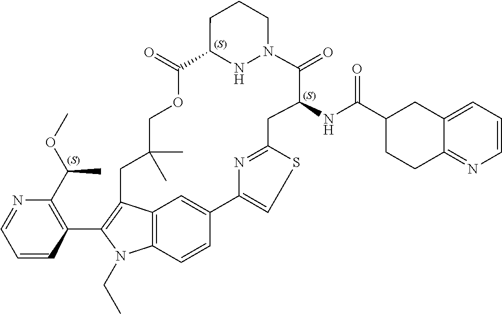 |
| DA75 | 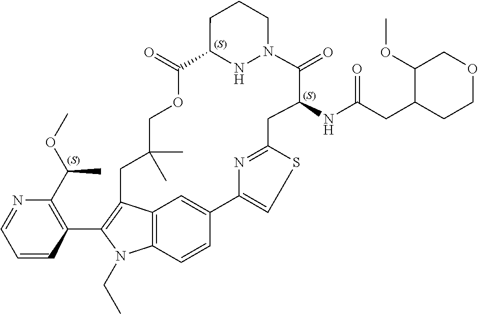 |
| DA76 | 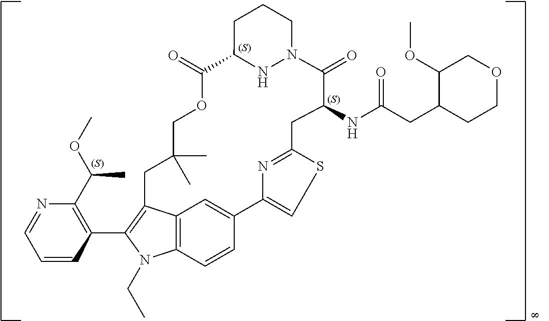 |
| DA77 | 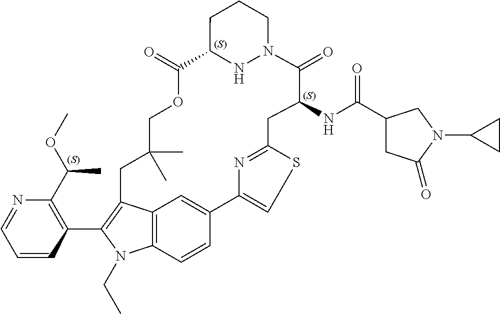 |
| DA78 | 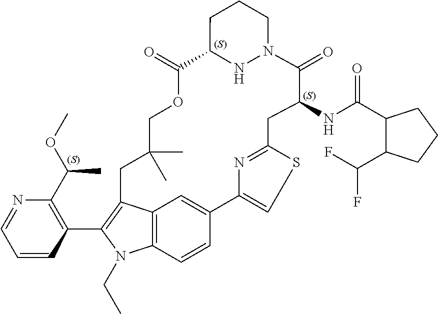 |
| DA79 | 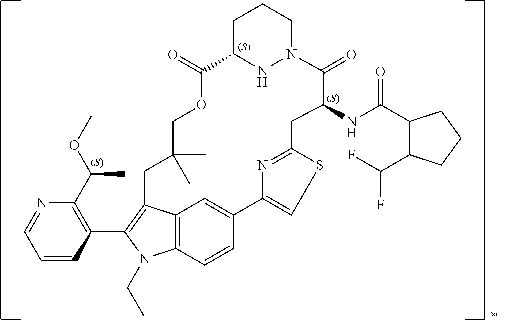 |
| DA80 | 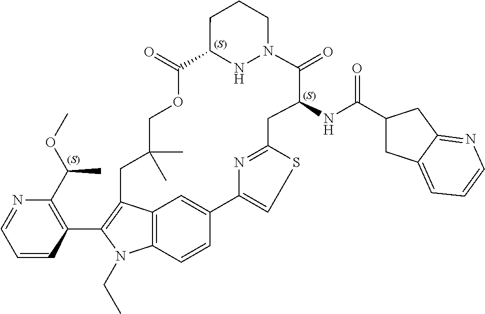 |
| DA81 | 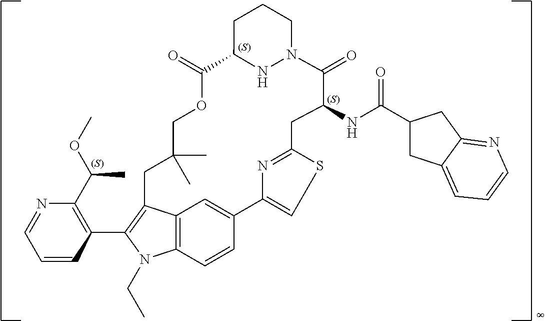 |
| DA82 | 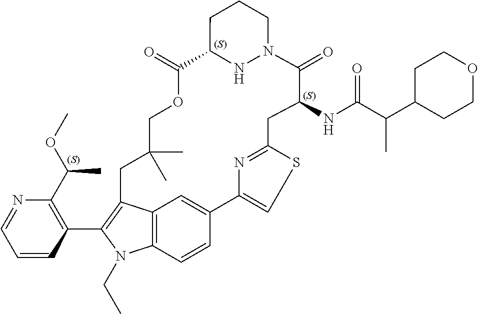 |
| DA83 | 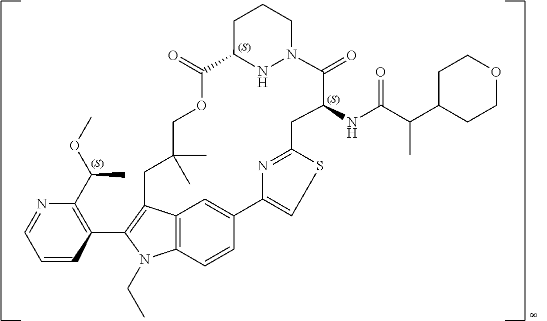 |
| DA84 | 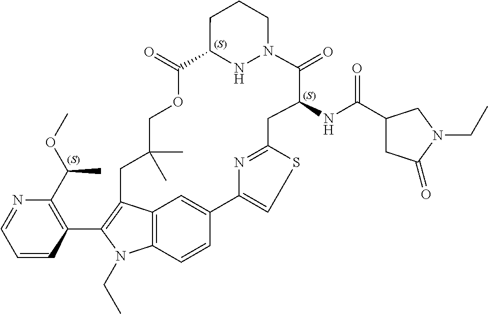 |
| DA85 | 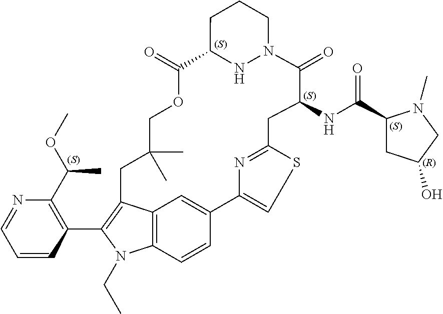 |
| DA86 | 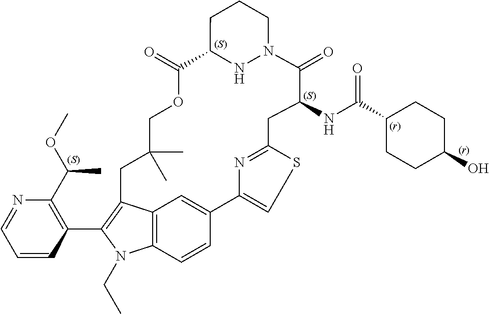 |
| DA87 | 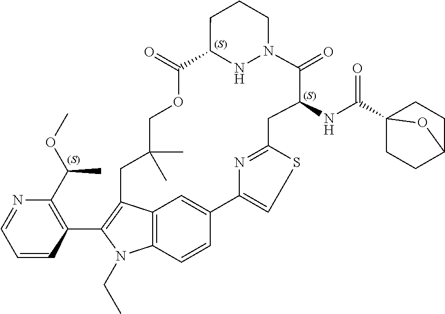 |
| DA88 | 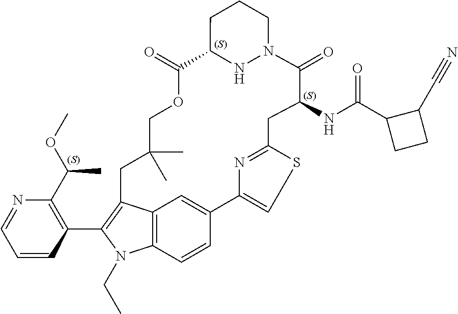 |
| DA89 | 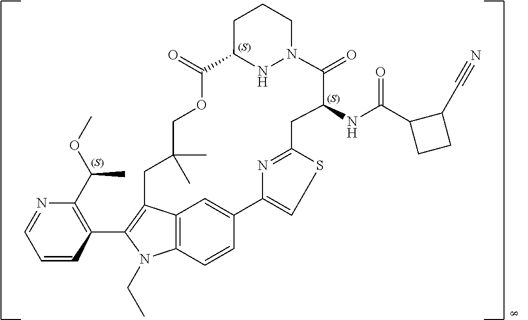 |
| DA90 | 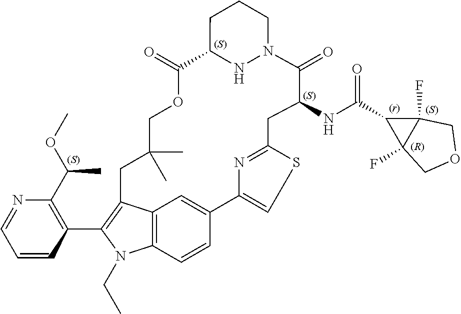 |
| DA91 | 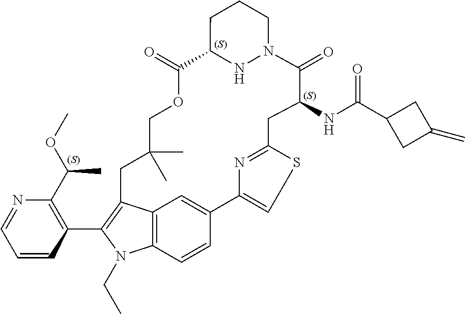 |
| DA92 | 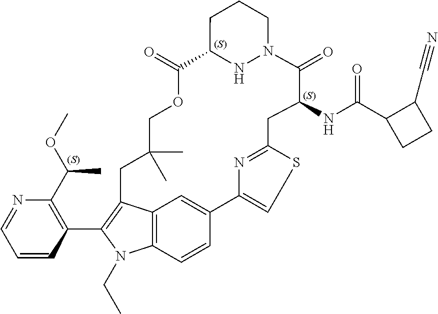 |
| DA93 | 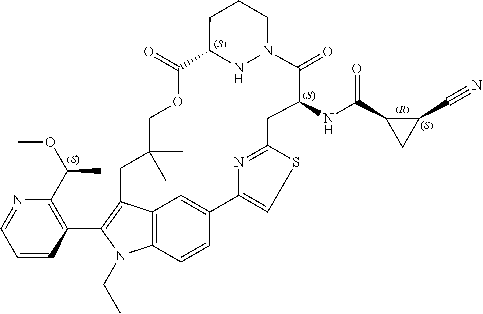 |
| DA94 | 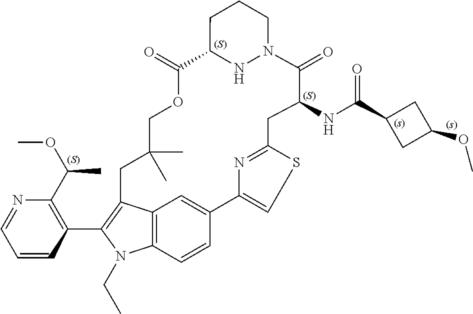 |
| DA95 | 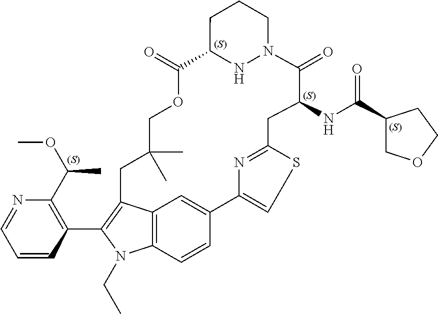 |
| DA96 | 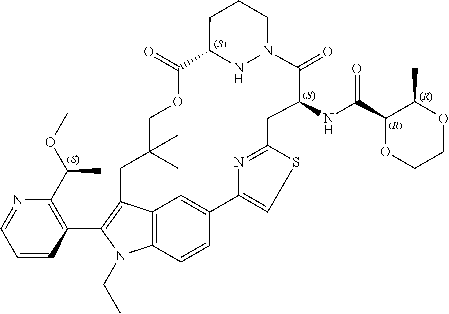 |
| DA97 | 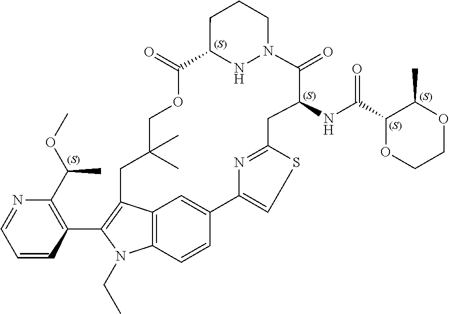 |
| DA98 | 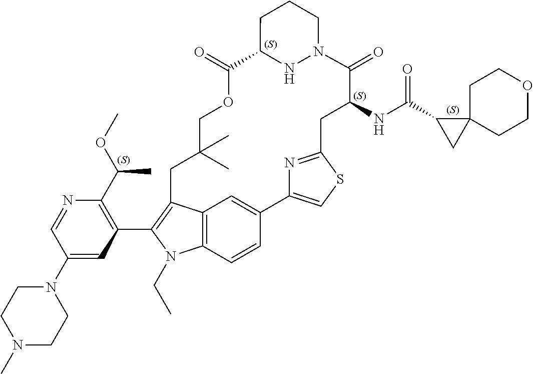 |
| DA99 | 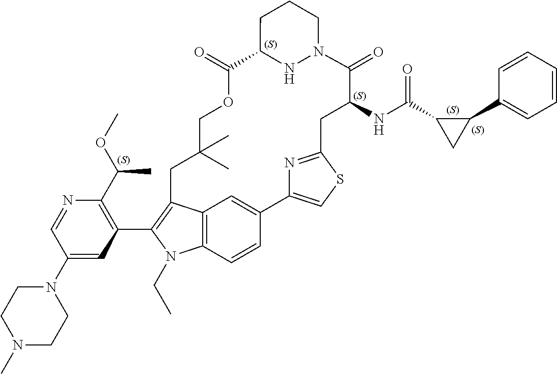 |
| DA100 | 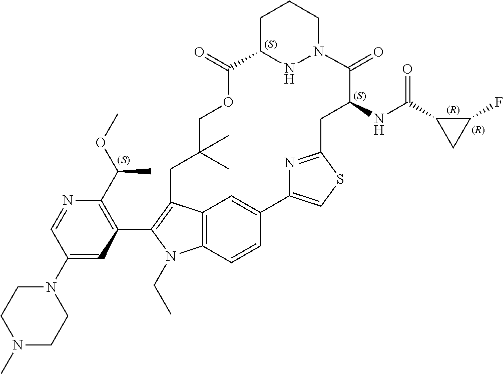 |
| DA101 | 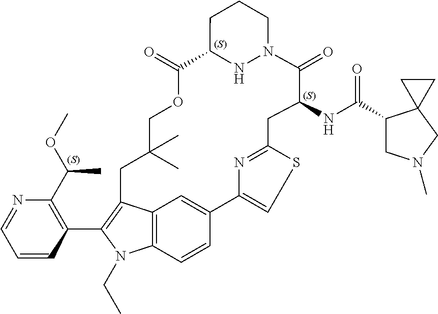 |
| DA102 | 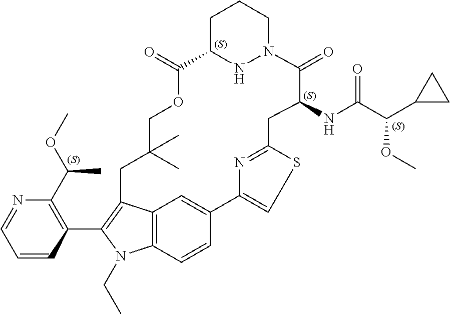 |
| DA103 | 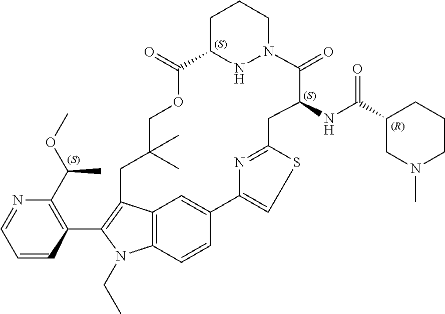 |
| DA104 | 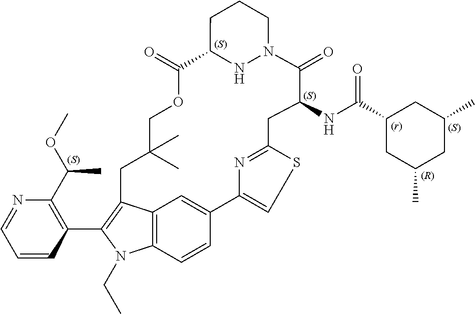 |
| DA105 | 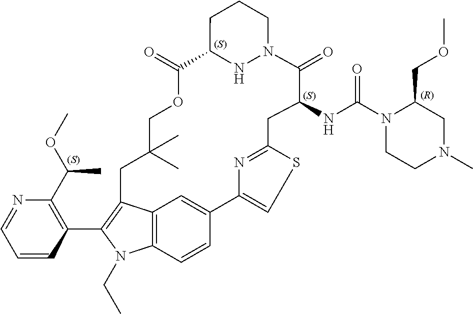 |
| DA106 | 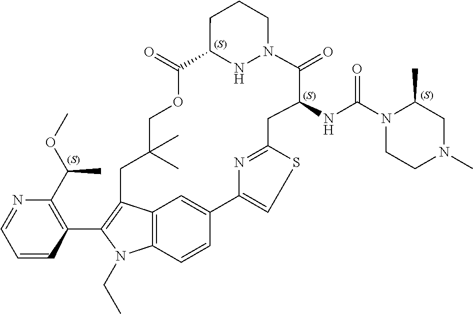 |
| DA107 | 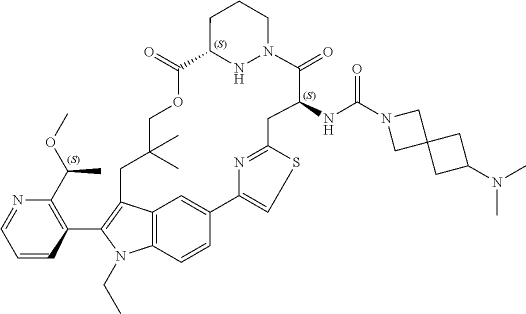 |
| DA108 | 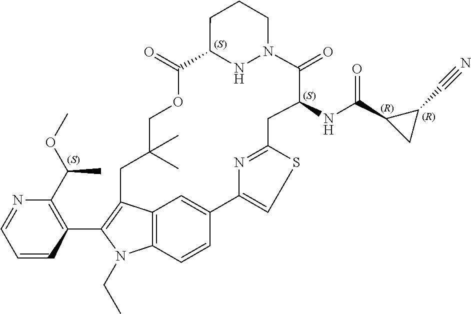 |
| DA109 | 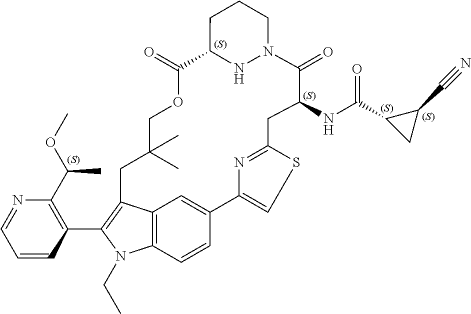 |
| DA110 | 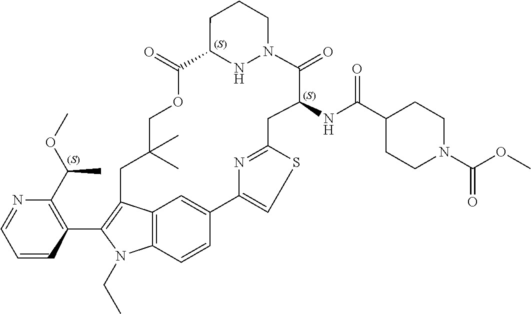 |
| DA111 | 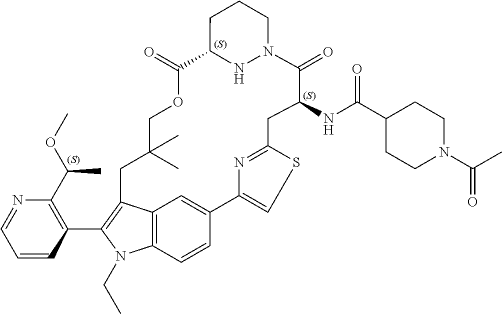 |
| DA112 | 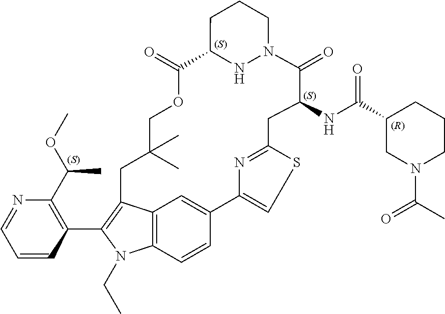 |
| DA113 | 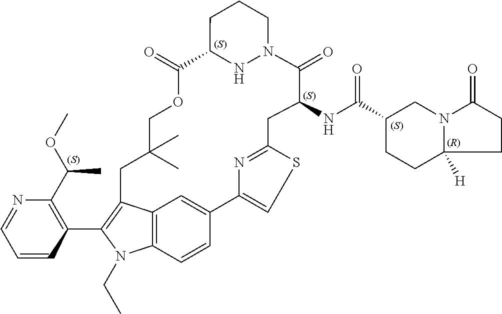 |
| DA114 | 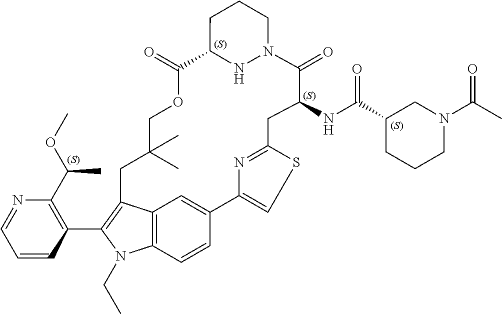 |
| DA115 | 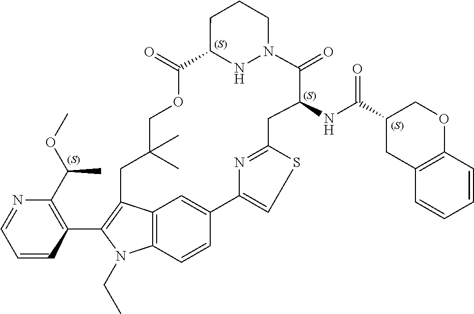 |
| DA116 | 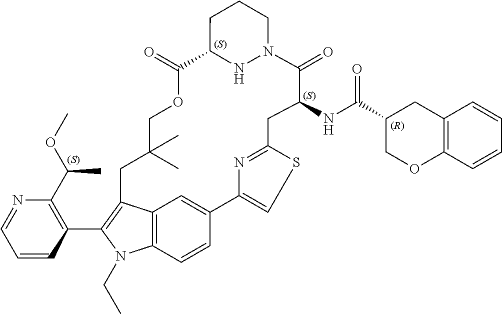 |
| DA117 | 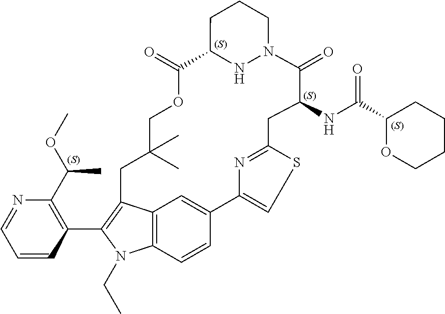 |
| DA118 | 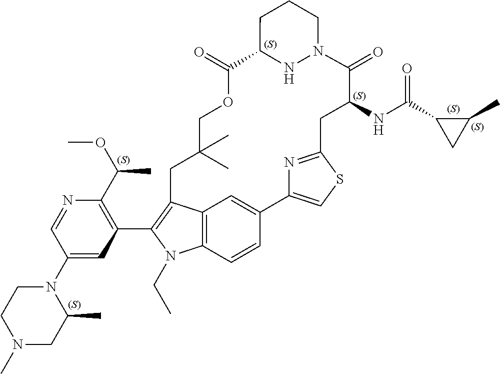 |
| DA119 | 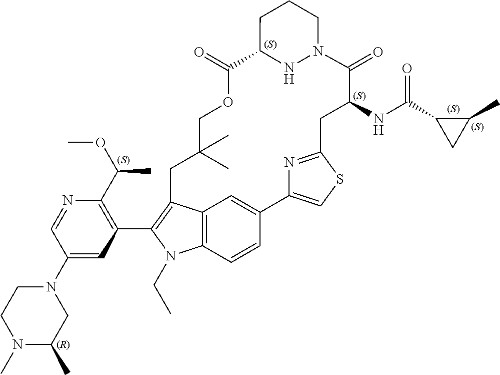 |
| DA120 | 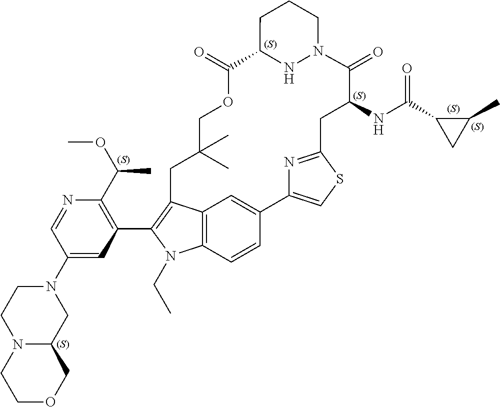 |
| DA121 | 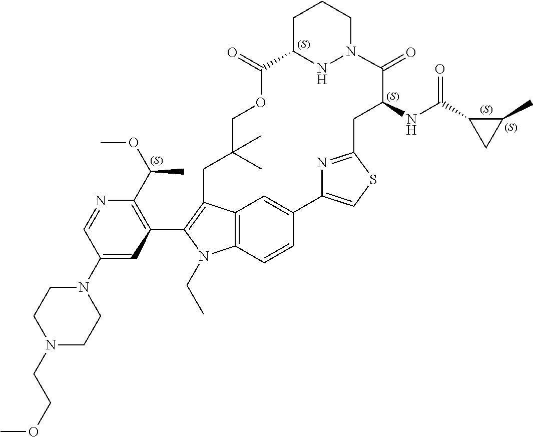 |
| DA122 | 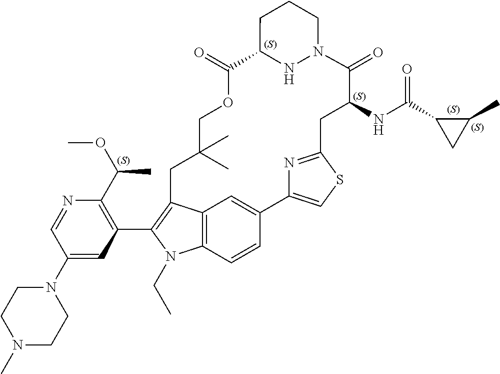 |
| DA123 | 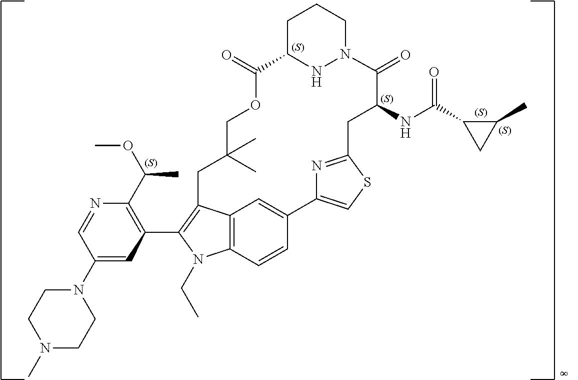 |
| DA124 | 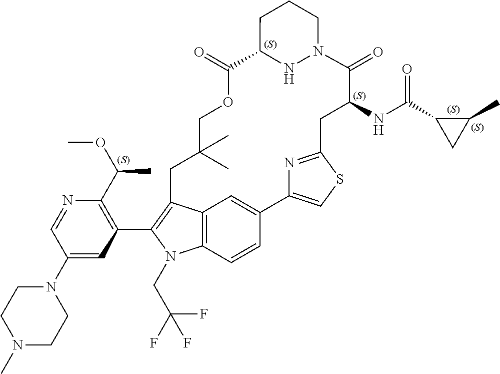 |
| DA125 | 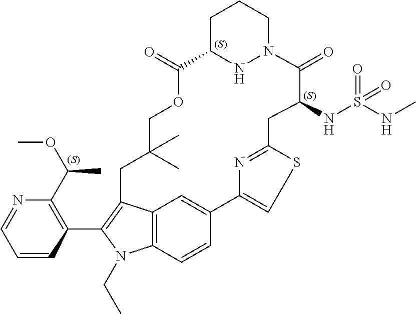 |
| DA126 | 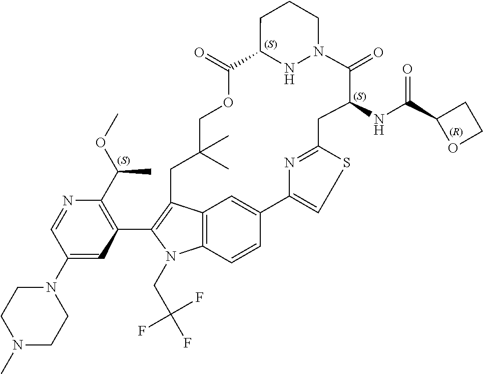 |
| DA127 | 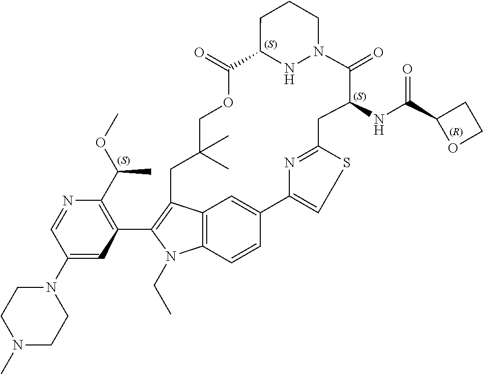 |
| DA128 | 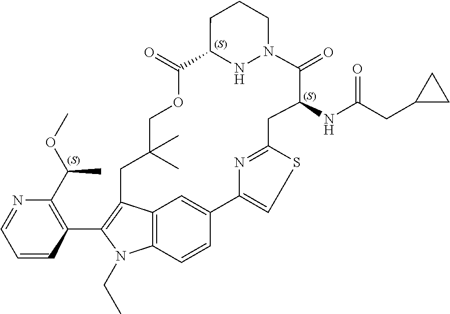 |
| DA129 | 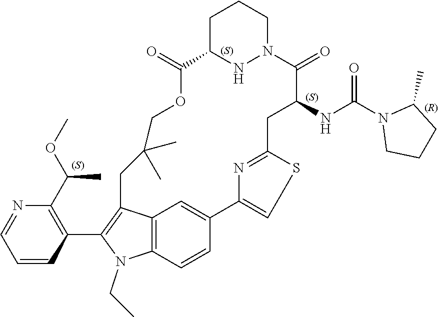 |
| DA130 | 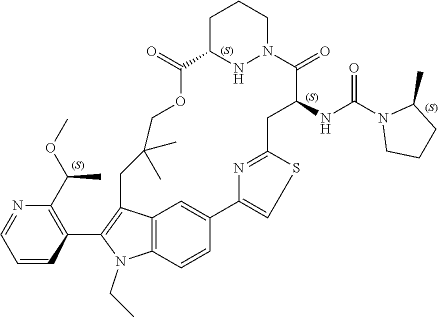 |
| DA131 | 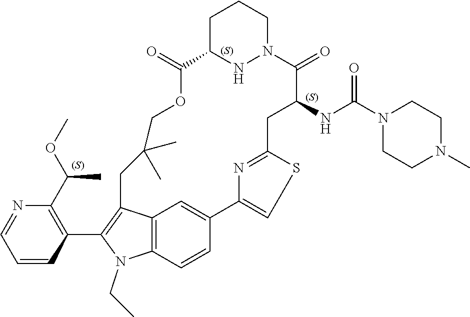 |
| DA132 | 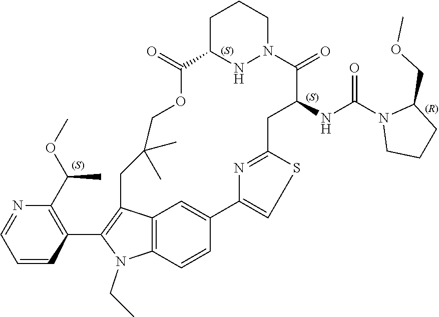 |
| DA133 | 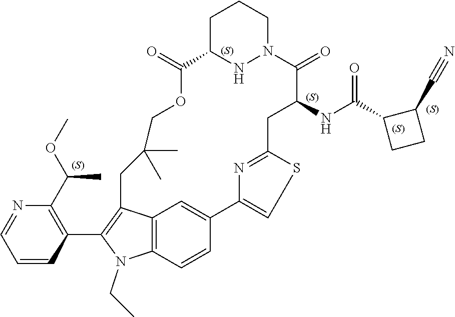 |
| DA133 | 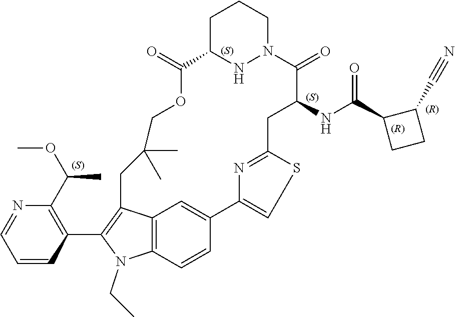 |
| DA134 | 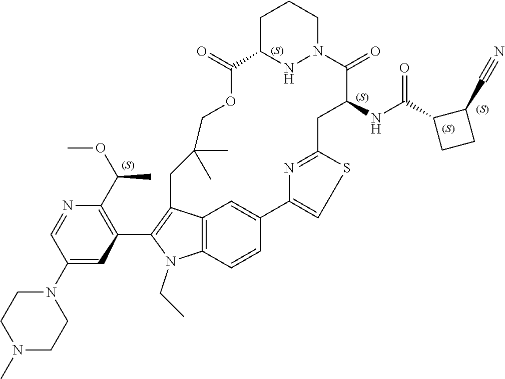 |
| DA135 | 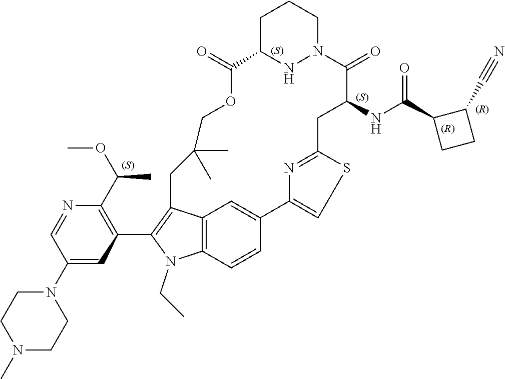 |
| DA136 | 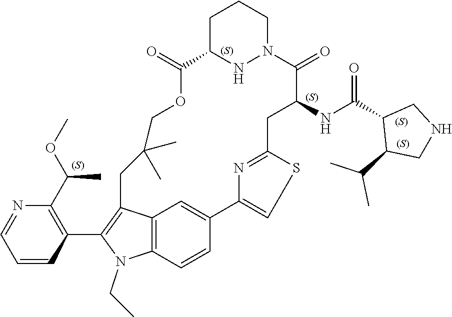 |
| DA137 | 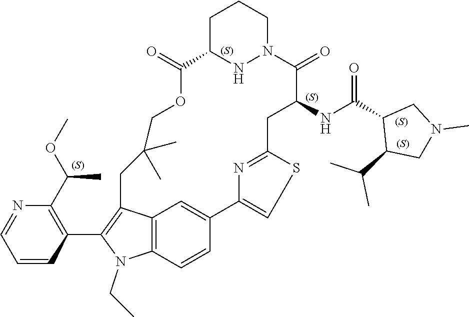 |
| DA138 | 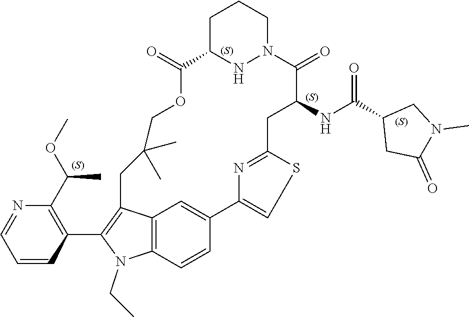 |
| DA139 | 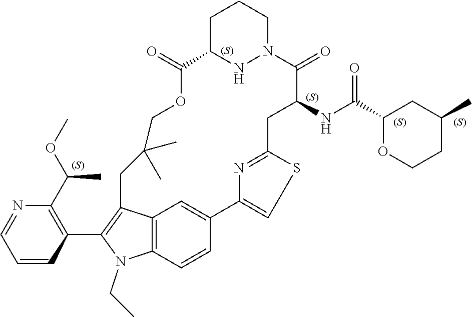 |
| DA140 | 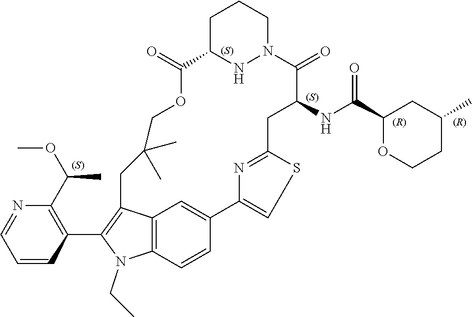 |
| DA141 | 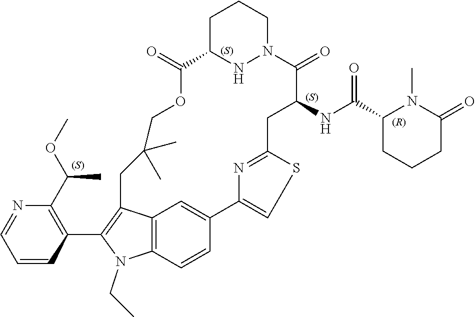 |
| DA142 | 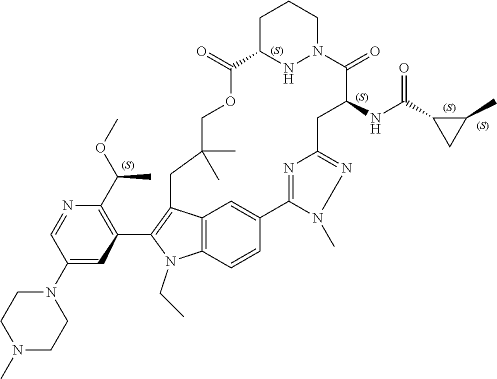 |
| DA143 | 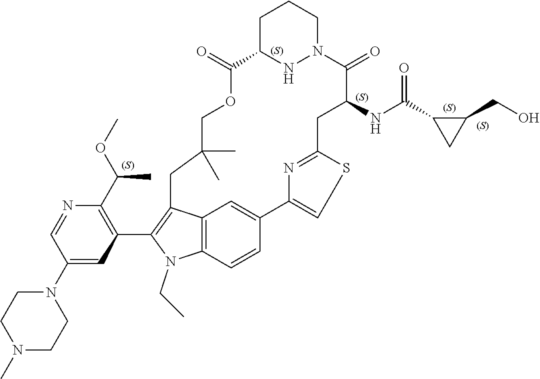 |
| DA144 | 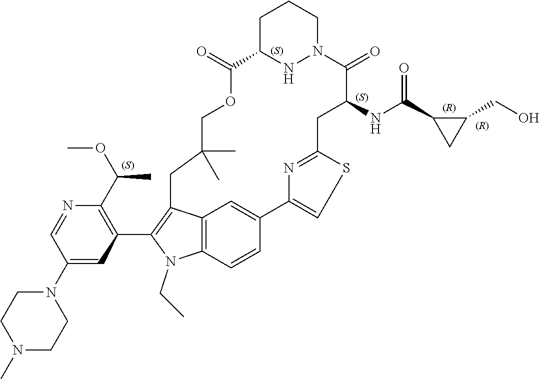 |
| DA145 | 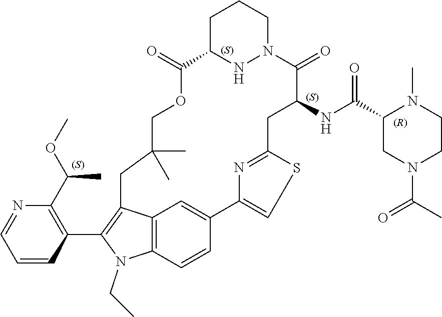 |
| DA146 | 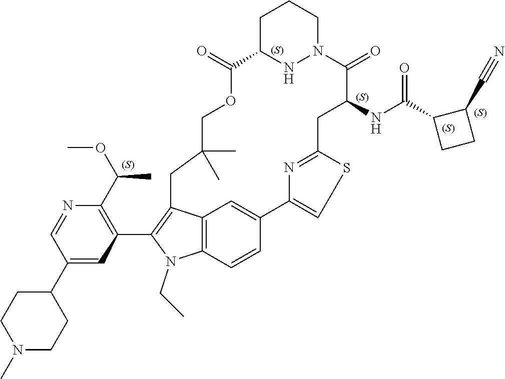 |
| DA147 | 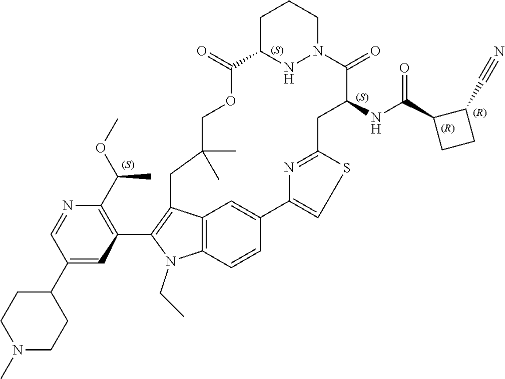 |
| DA148 | 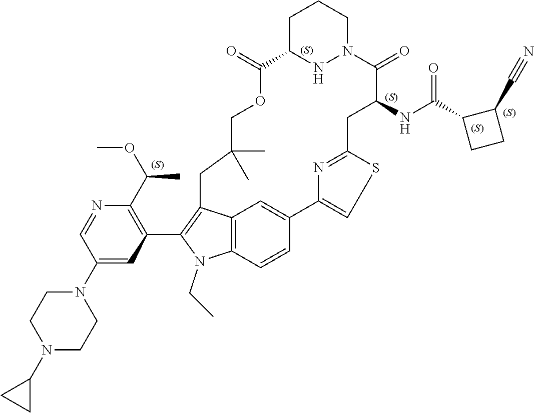 |
| DA149 | 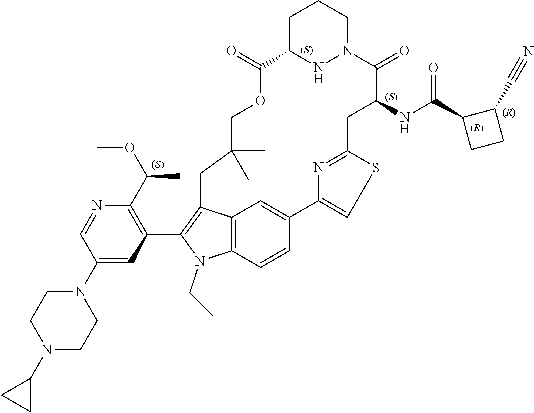 |
| DA150 | 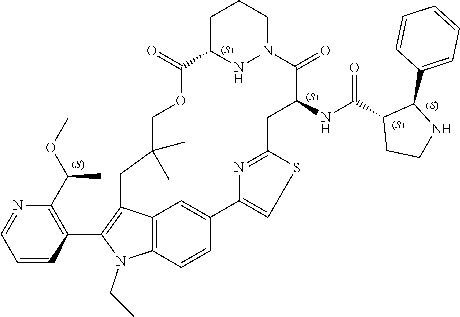 |
| DA151 | 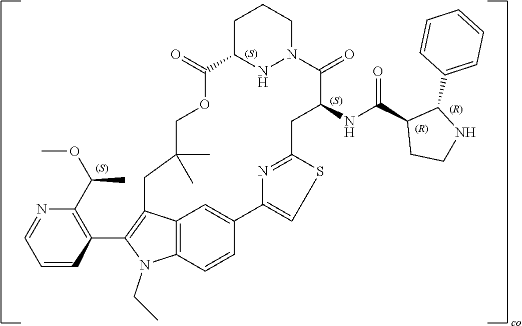 |
| DA152 | 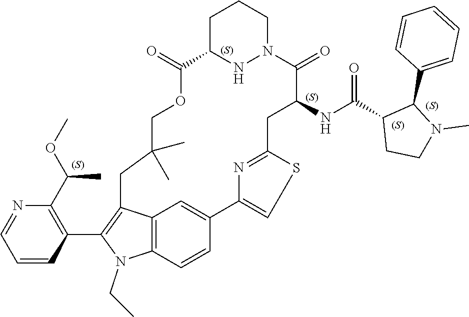 |
| DA153 | 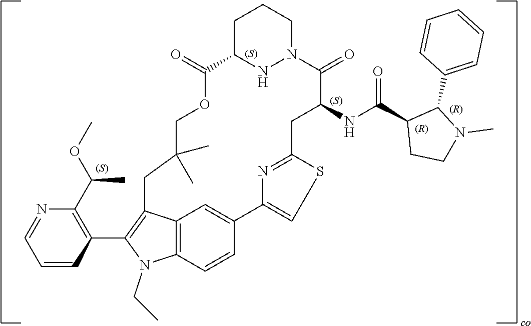 |
| DA154 | 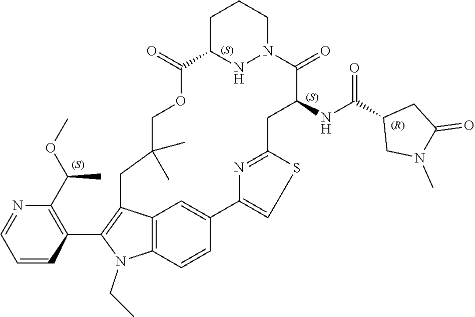 |
| DA155 | 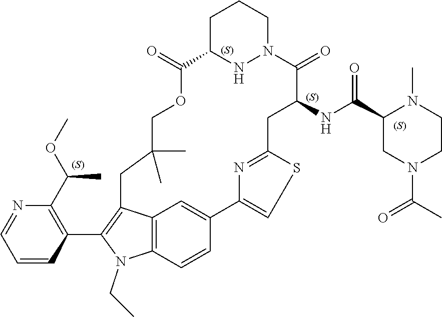 |
| DA156 | 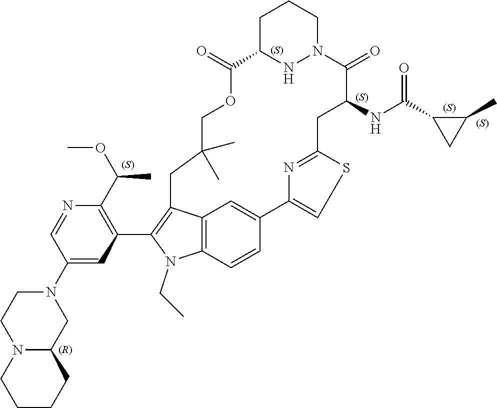 |
| DA157 | 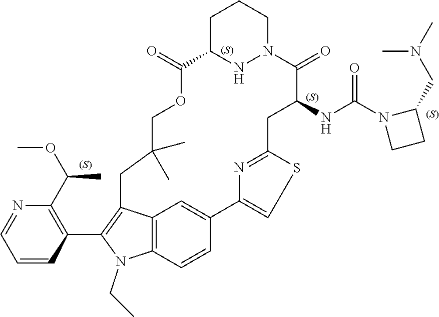 |
| DA158 | 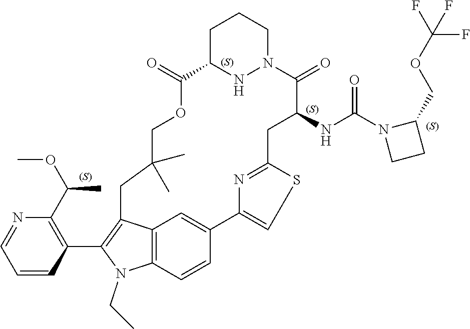 |
| DA159 | 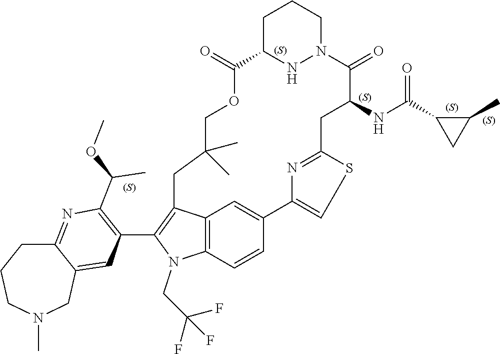 |
| DA160 | 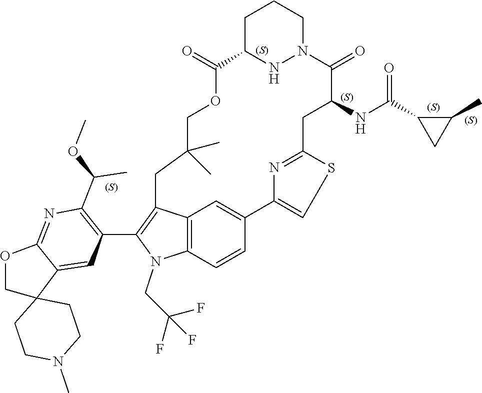 |
| DA161 | 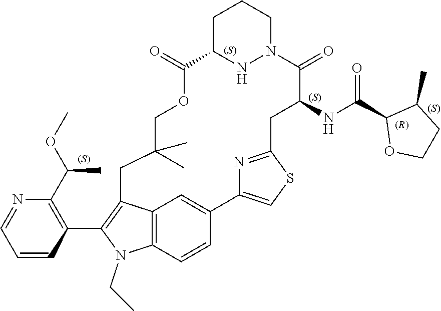 |
| DA162 | 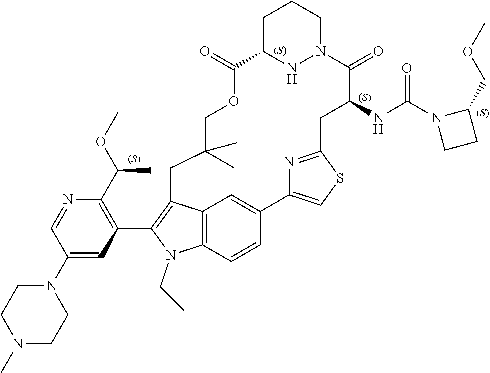 |
| DA163 | 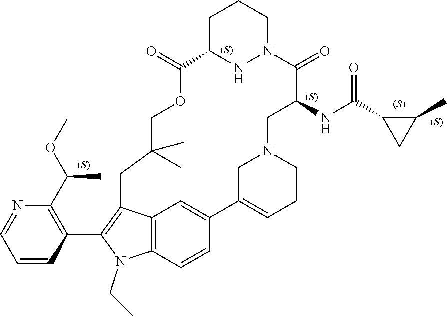 |
| DA164 | 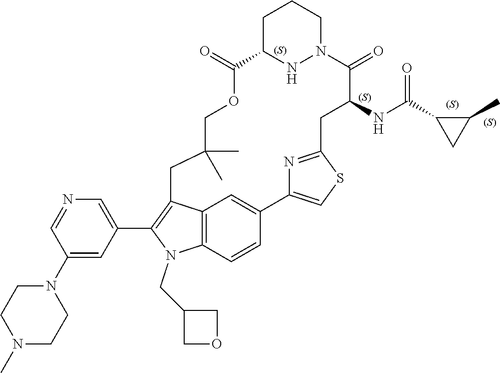 |
| DA165 | 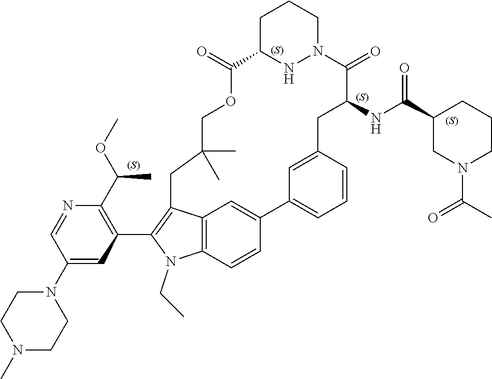 |
| DA166 | 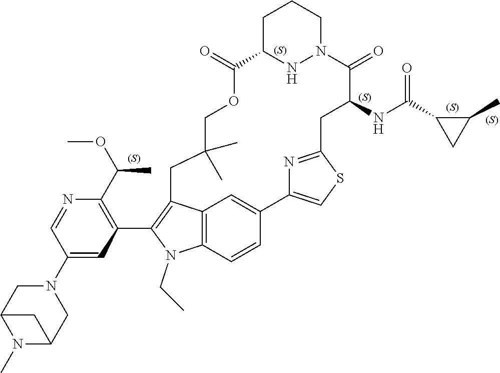 |
| DA167 | 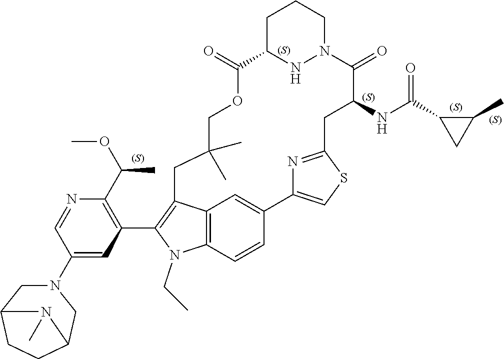 |
| DA168 | 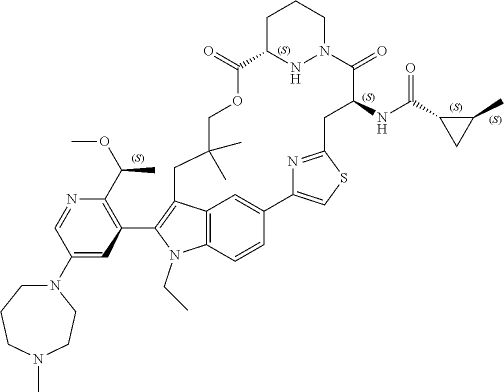 |
| DA169 | 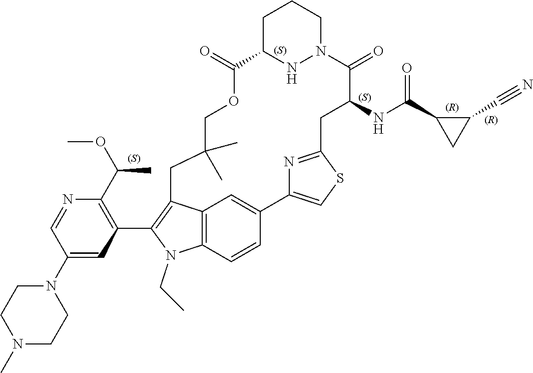 |
| DA170 | 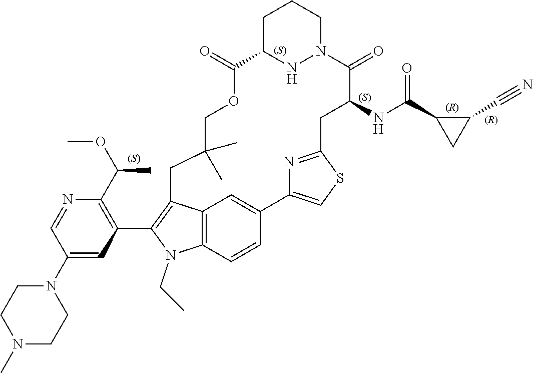 |
| DA171 | 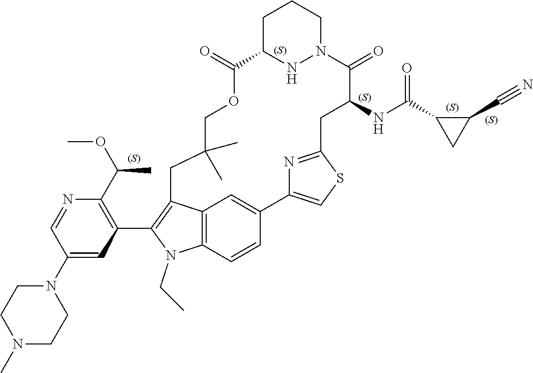 |
| DA172 | 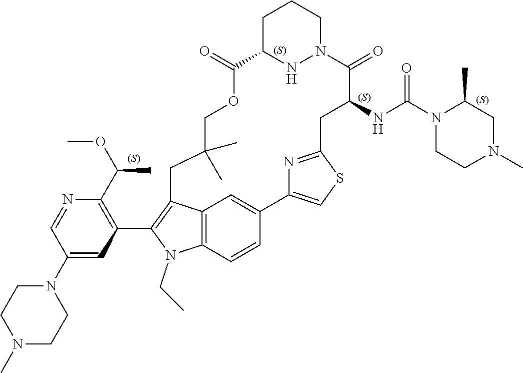 |
| DA173 | 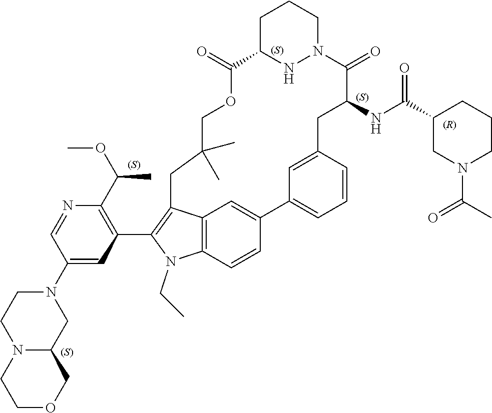 |
| DA174 | 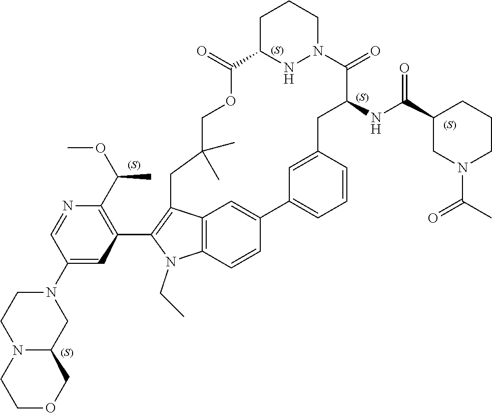 |
| DA175 | 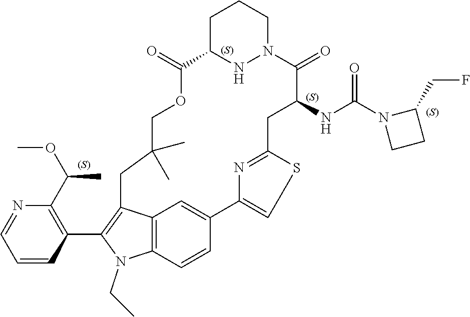 |
| DA176 | 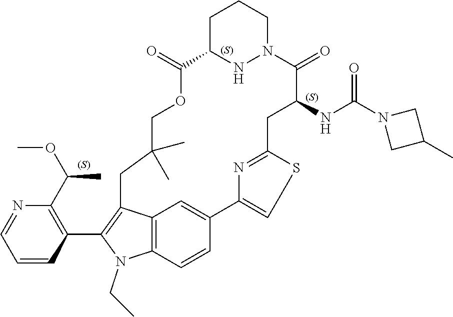 |
| DA177 | 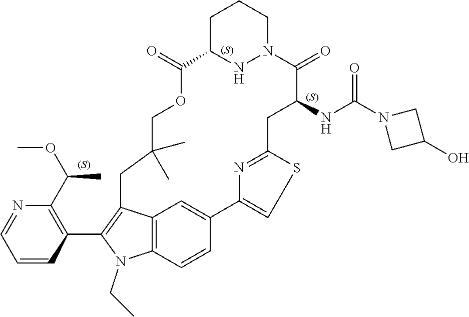 |
| DA178 | 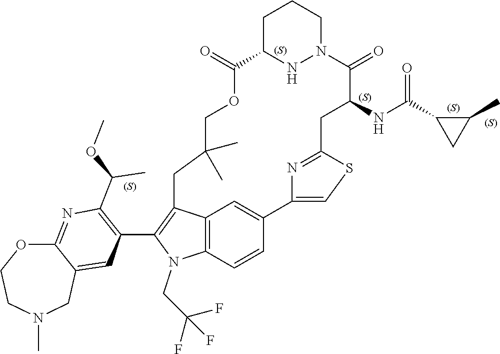 |
| DA179 | 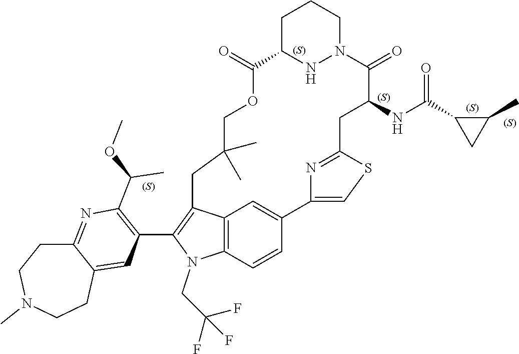 |
| DA180 | 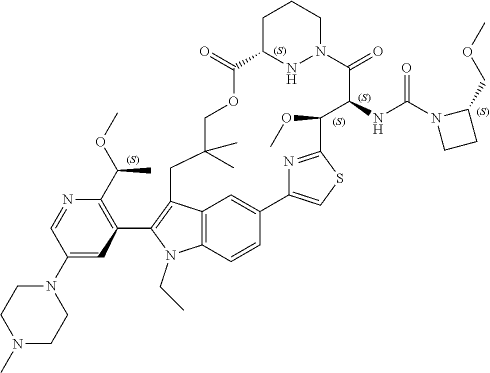 |
| DA181 | 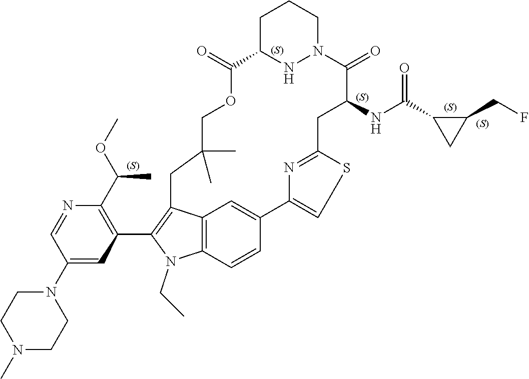 |
| DA182 | 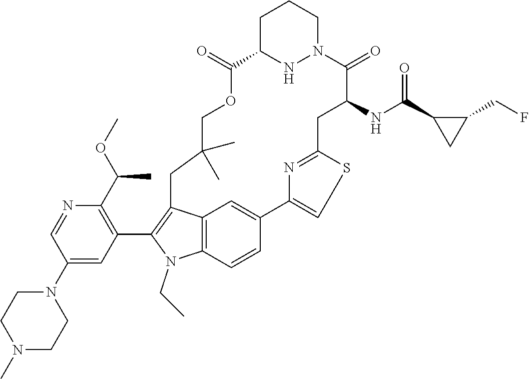 [SMILES: CCn1c(-c2cc(N3CCN(C)CC3)cnc2[C@H](C)OC)c2c3cc(ccc31)-c1csc(n1)C[C@H](NC(=O)[C@@H]1C[C@H]1CF)C(=O)N1CCC[C@H](N1)C(=O)OCC(C)(C)C2] |
| DA183 | 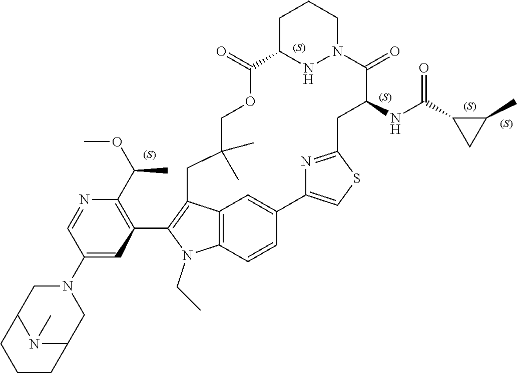 |
| DA184 | 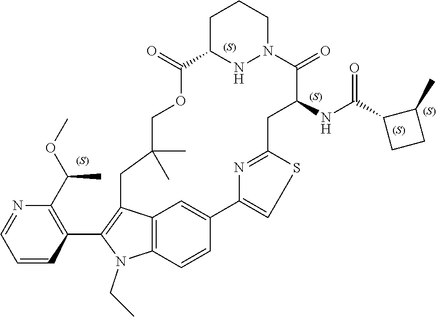 |
| DA185 | 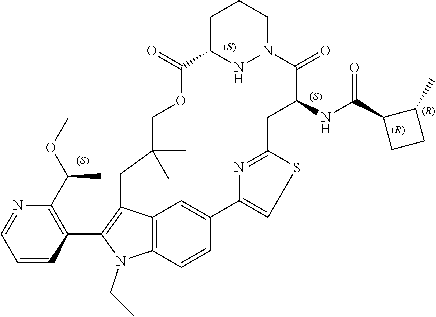 |
| DA186 | 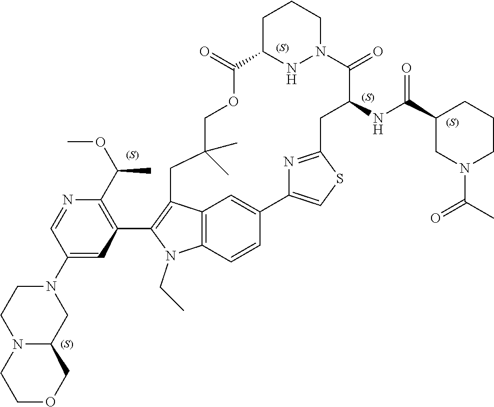 |
| DA187 | 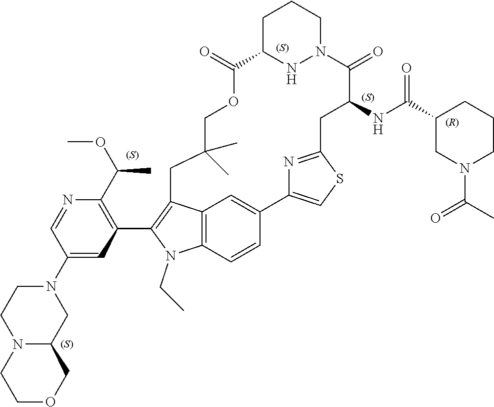 |
| DA188 | 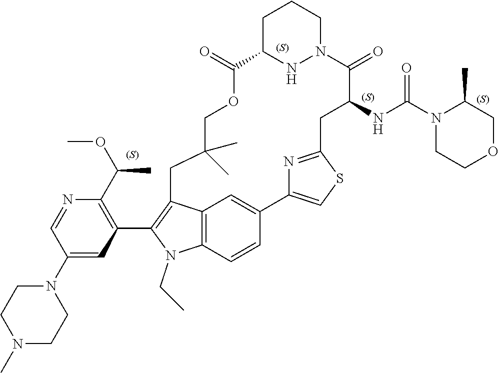 |
| DA189 | 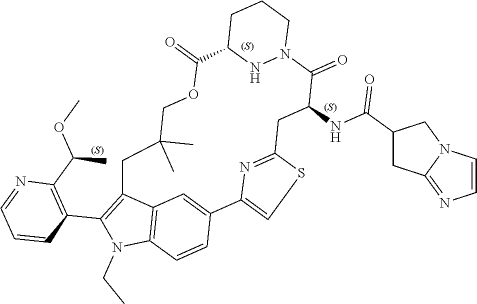 |
| DA190 | 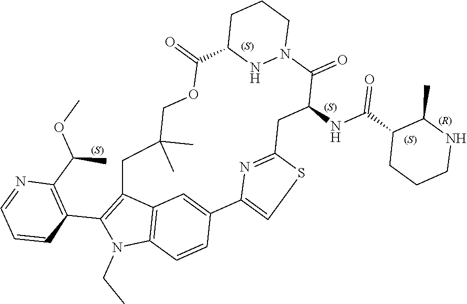 |
| DA191 | 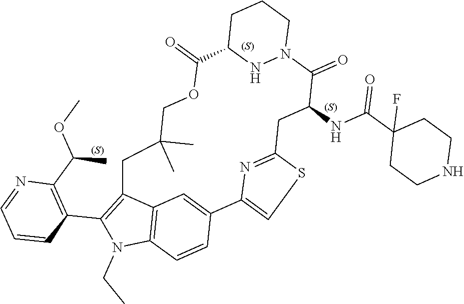 |
| DA192 | 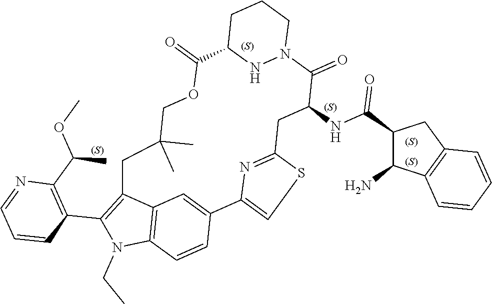 |
| DA193 | 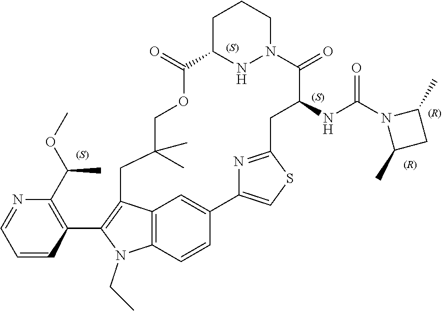 |
| DA194 | 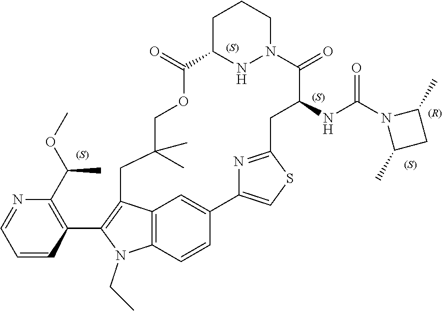 |
| DA195 | 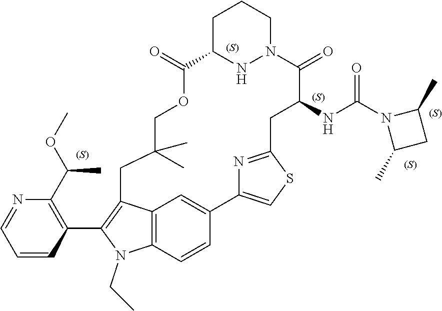 |
| DA196 | 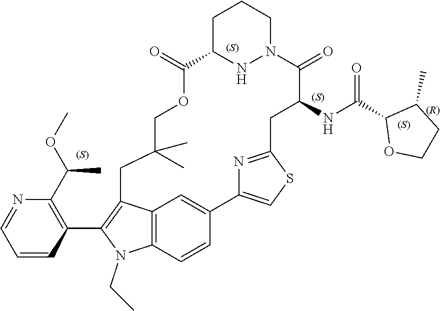 |
| DA197 | 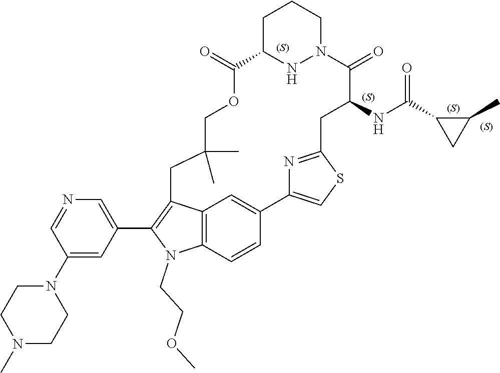 |
| DA198 | 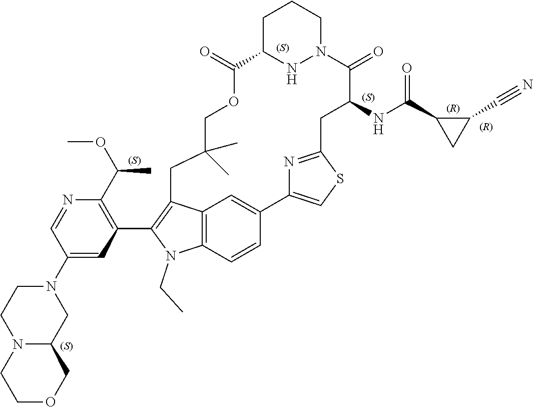 |
| DA199 | 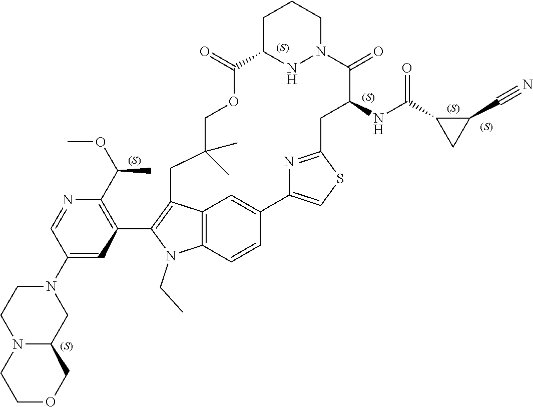 |
| DA200 | 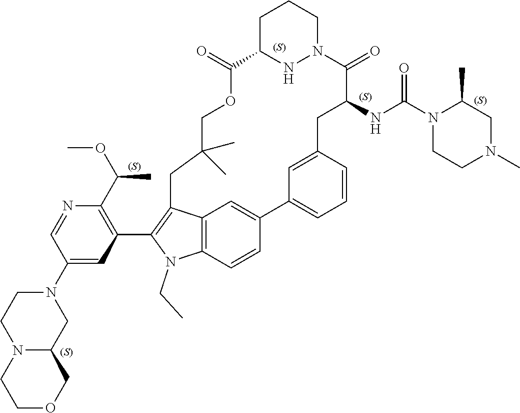 |
| DA201 | 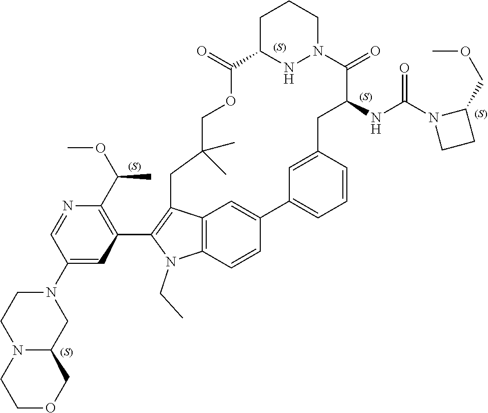 |
| DA202 | 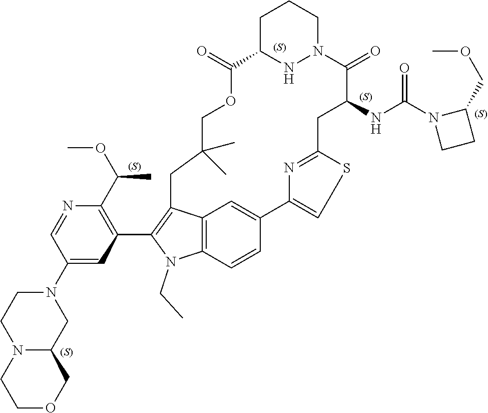 |
| DA203 | 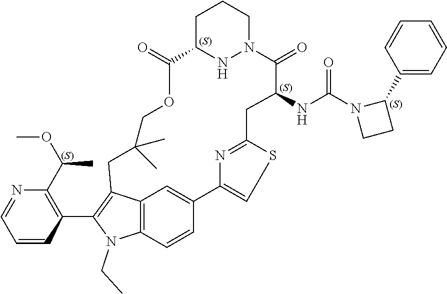 |
| DA204 | 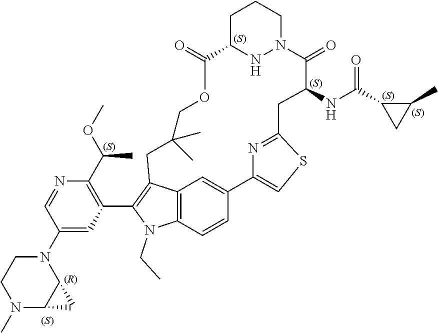 |
| DA205 | 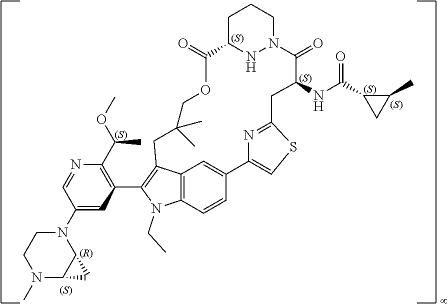 |
| DA206 | 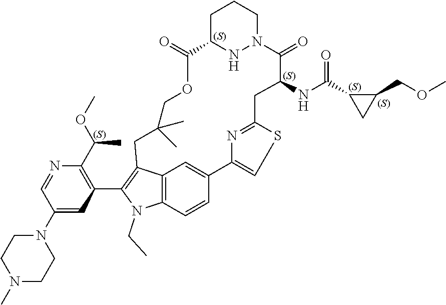 |
| DA207 | 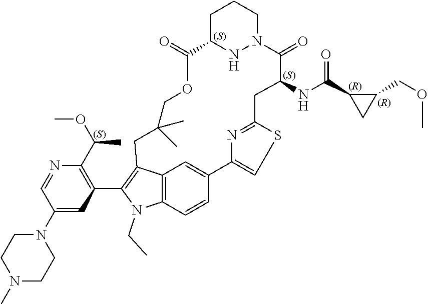 |
| DA208 | 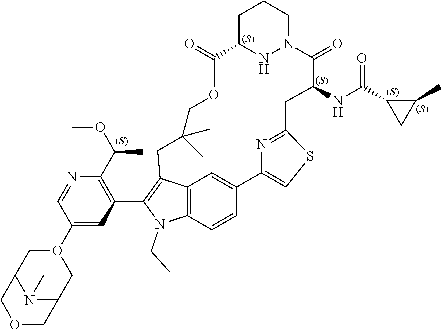 |
| DA209 | 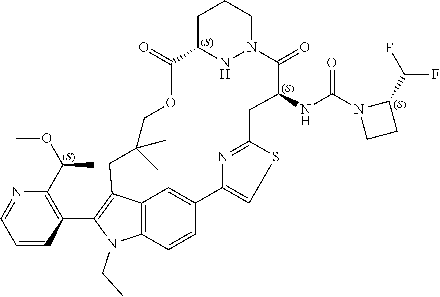 |
| DA210 | 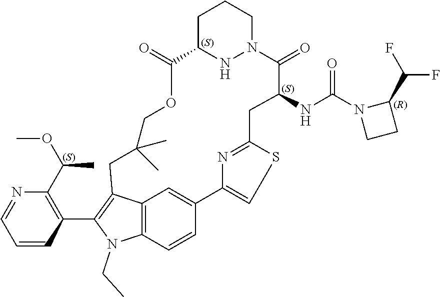 |
| DA211 | 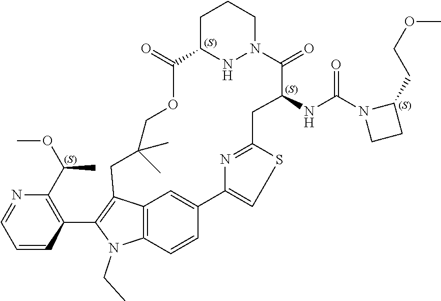 |
| DA212 | 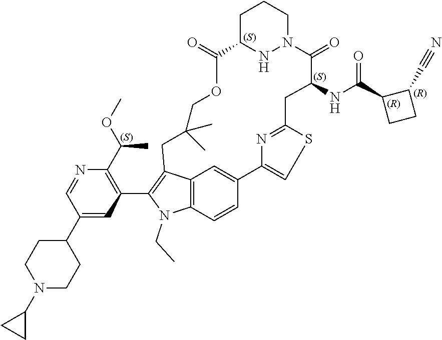 |
| DA213 | 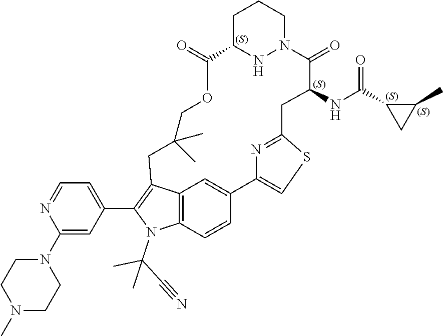 |
| DA214 | 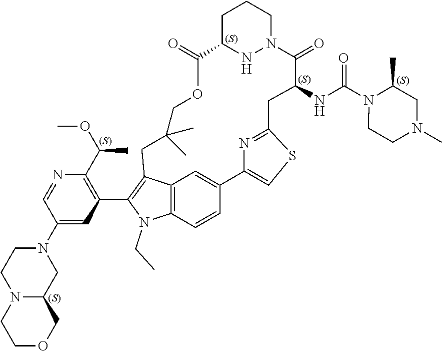 |
| DA215 | 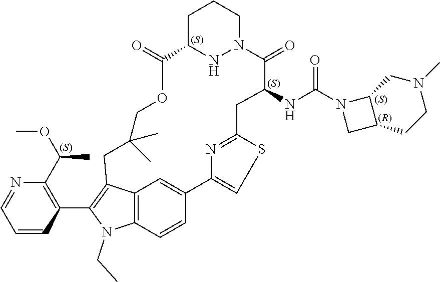 |
| DA216 | 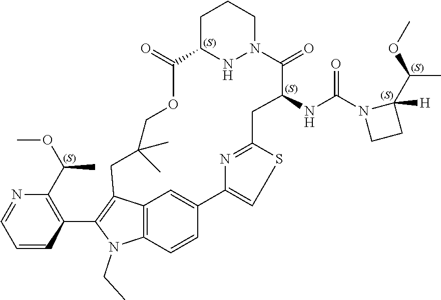 |
| DA217 | 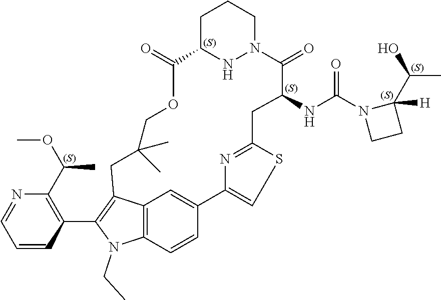 |
| DA218 | 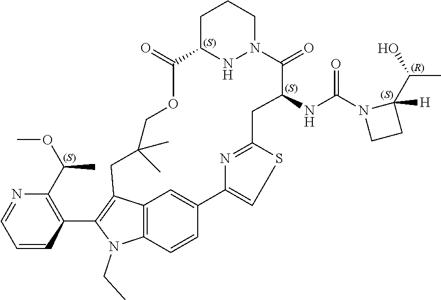 |
| DA219 | 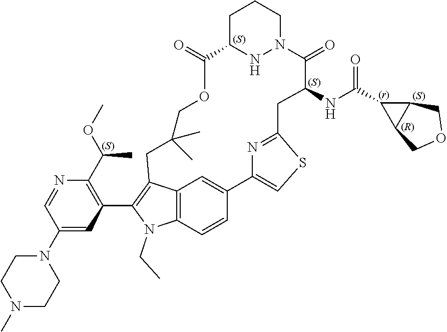 |
| DA220 | 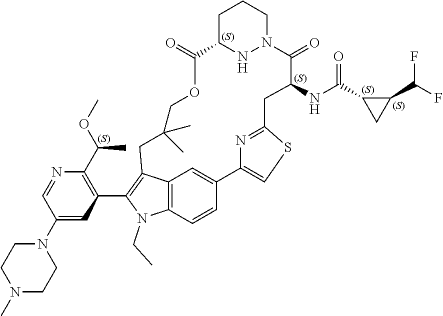 |
| DA221 | 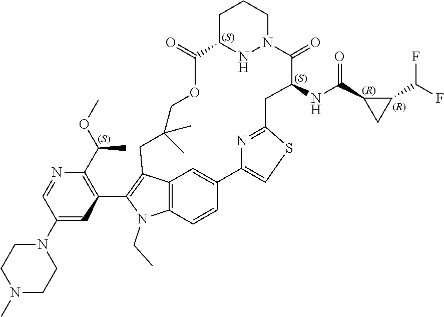 |
| DA222 | 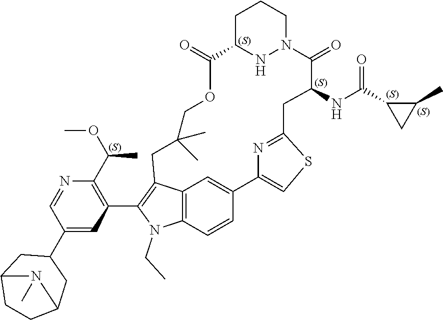 |
| DA223 | 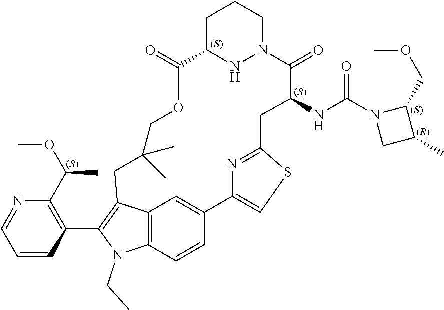 |
| DA224 | 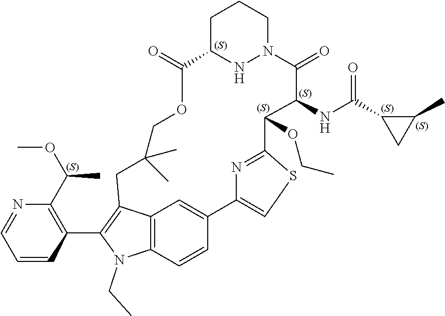 |
| DA225 | 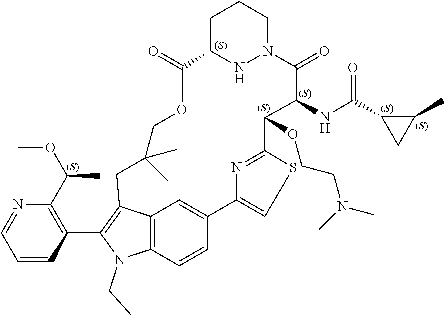 |
| DA226 | 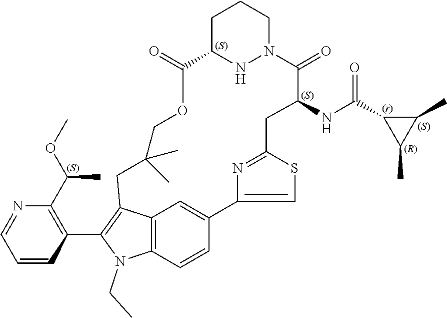 |
| DA227 | 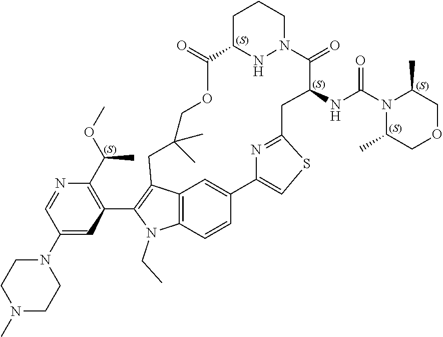 |
| DA228 | 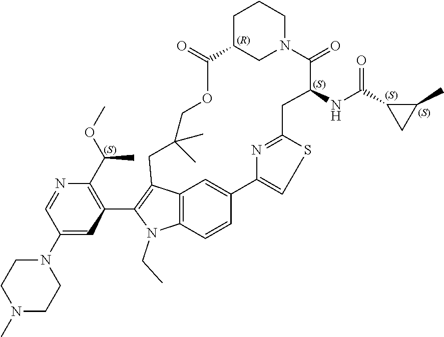 |
| DA229 | 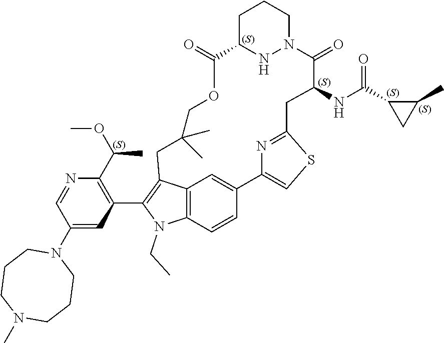 |
| DA230 | 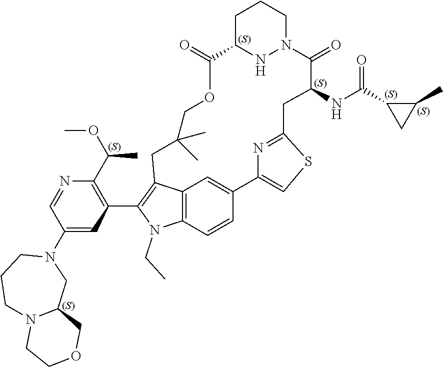 |
| DA231 | 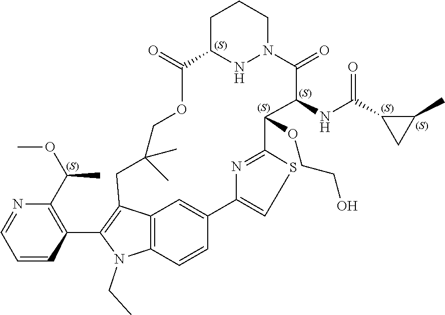 |
| DA232 | 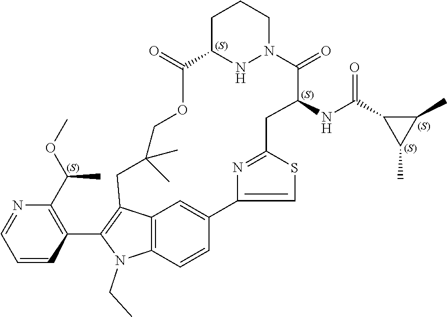 |
| DA233 | 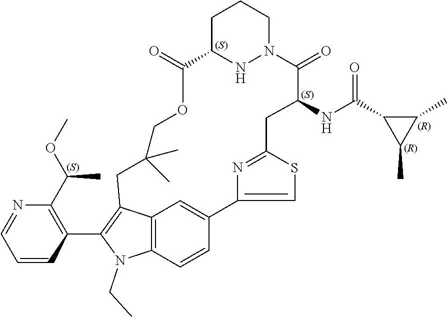 |
| DA234 | 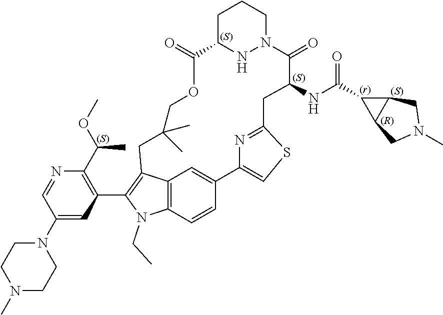 |
| DA235 | 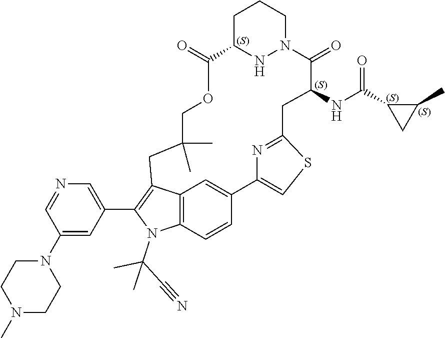 |
| DA236 | 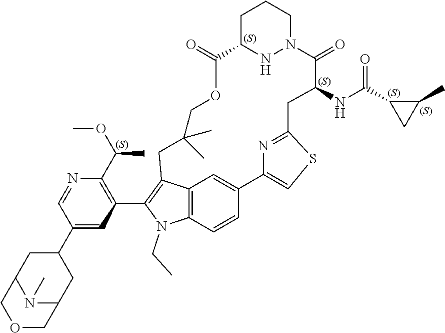 |
| DA237 | 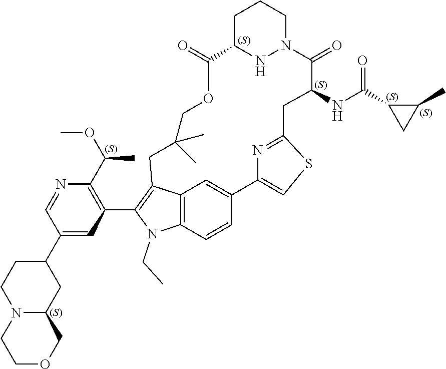 |
| DA238 | 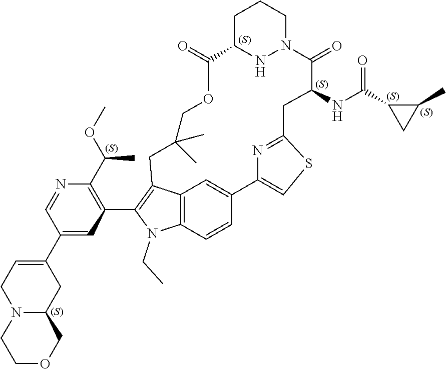 |
| DA239 | 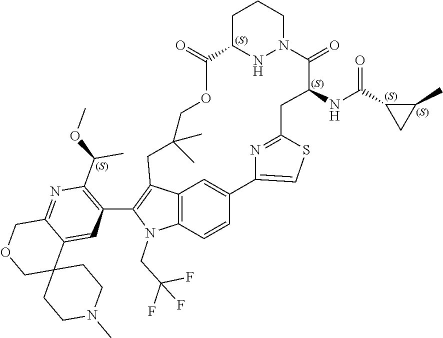 |
| DA240 | 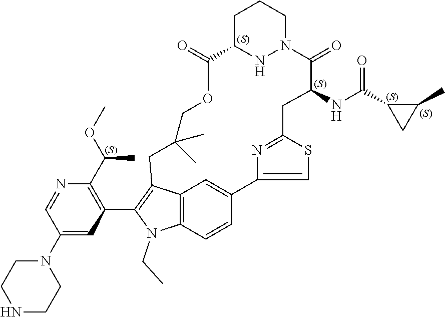 |
| DA241 | 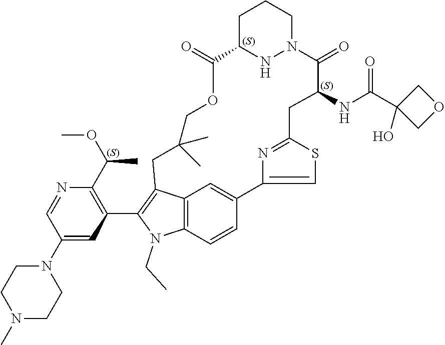 |
| DA242 | 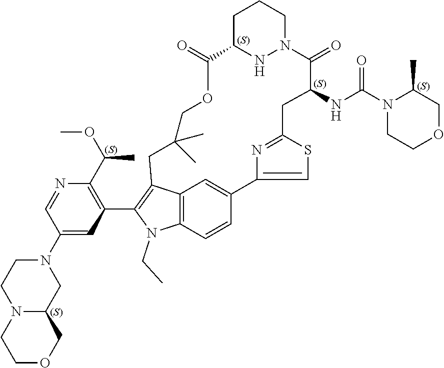 |
| DA243 | 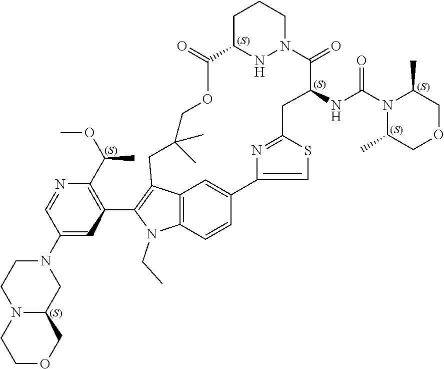 |
| DA244 | 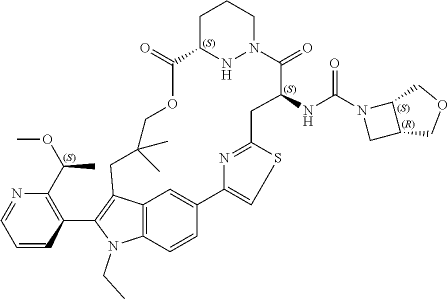 |
| DA245 | 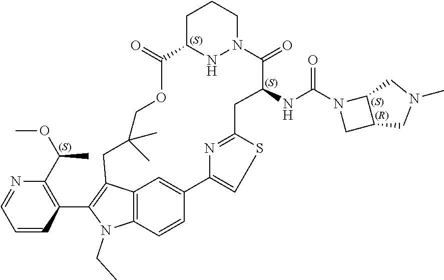 |
| DA246 | 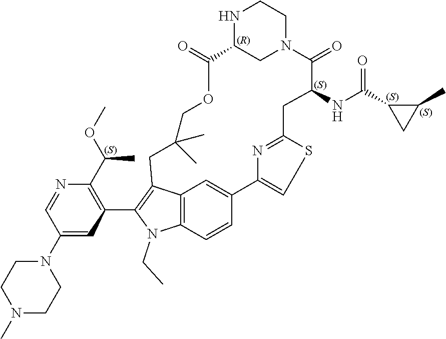 |
| DA247 | 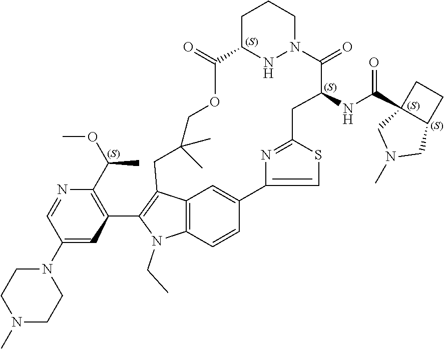 |
| DA248 | 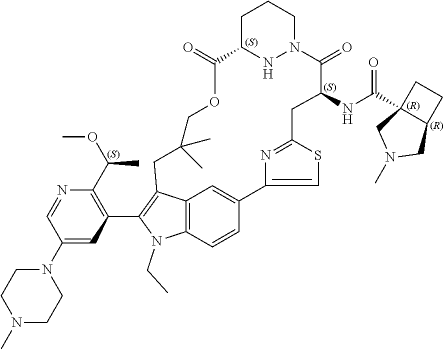 |
| DA249 | 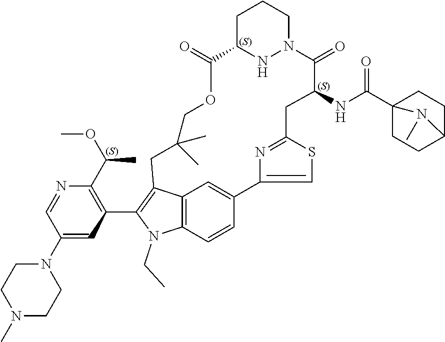 |
| DA250 | 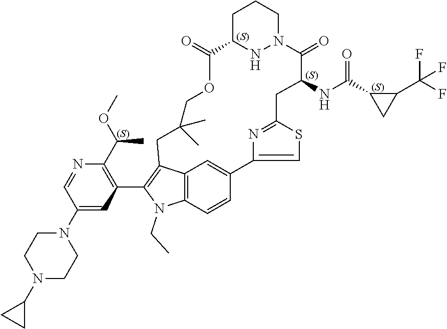 |
| DA251 | 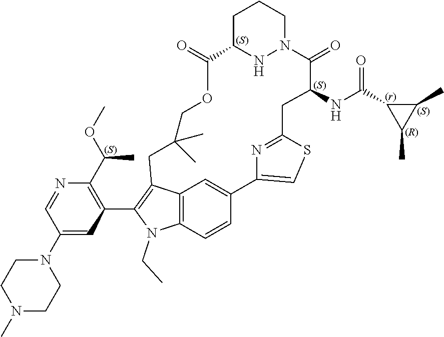 |
| DA252 | 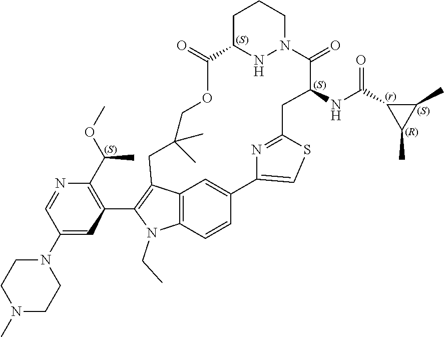 |
| DA253 | 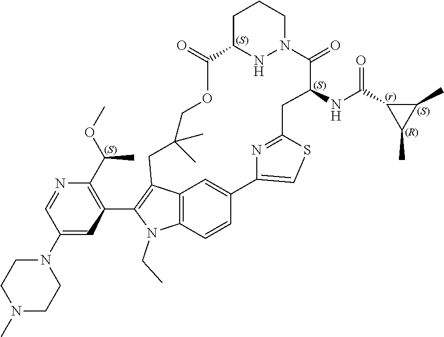 |
| DA254 | 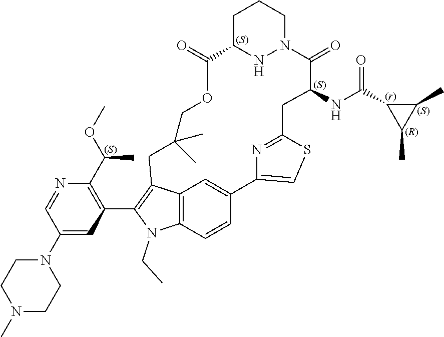 |
| DA255 | 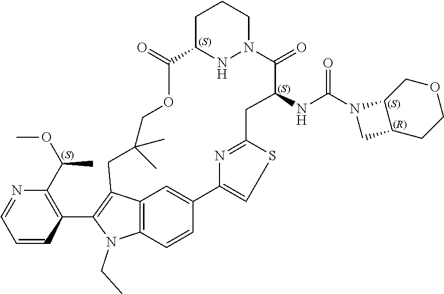 |
| DA256 | 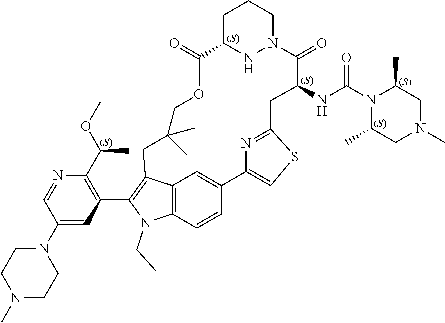 |
| DA257 | 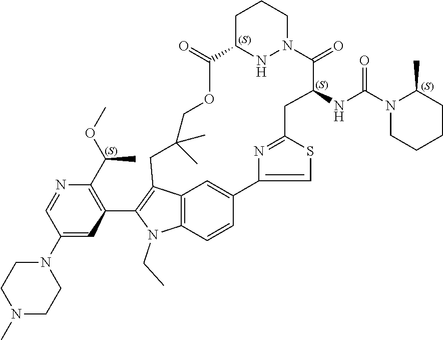 |
| DA258 | 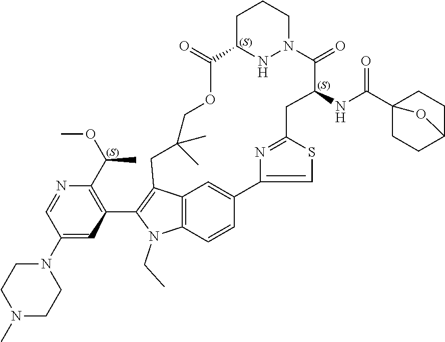 |
| DA259 | 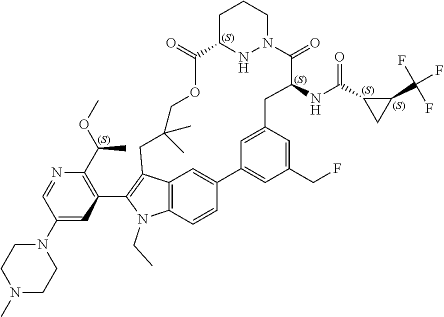 |
| DA260 | 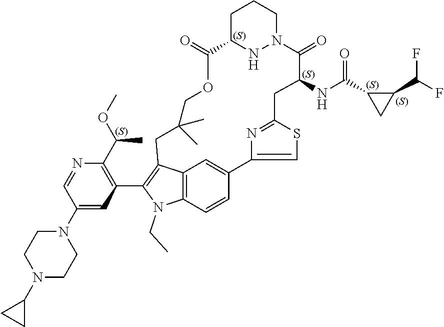 |
| DA261 | 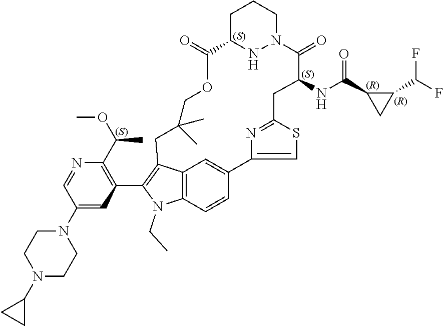 |
| DA262 | 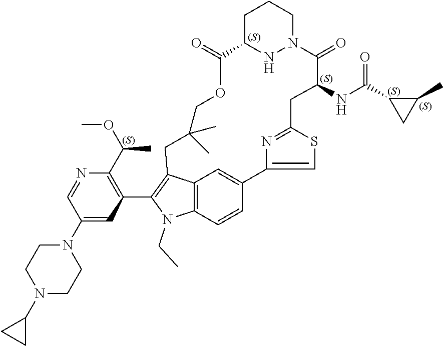 |
| DA263 | 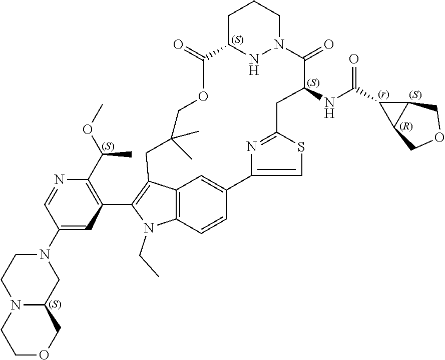 |
| DA264 | 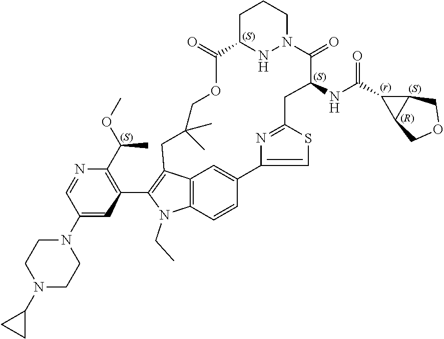 |
| DA265 | 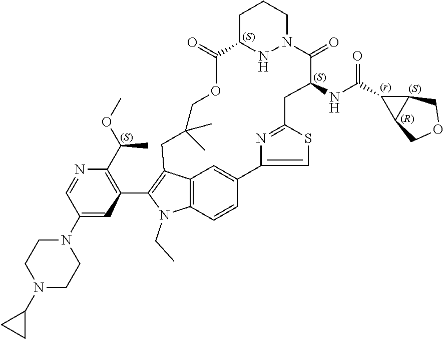 |
| DA266 | 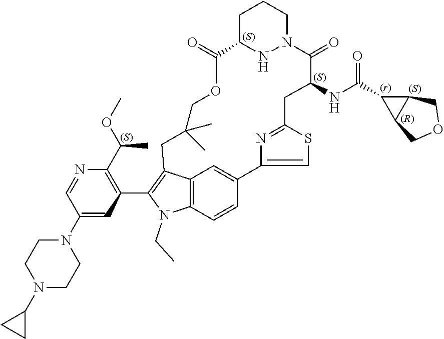 |
| DA267 | 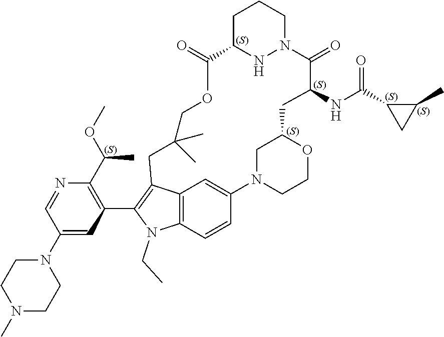 |
| DA268 | 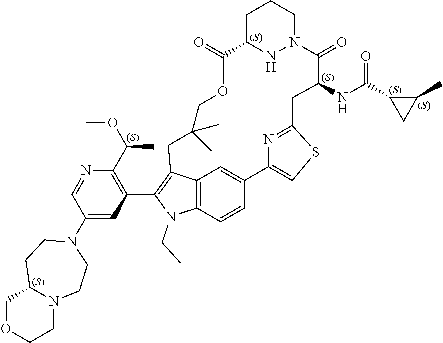 |
| DA269 | 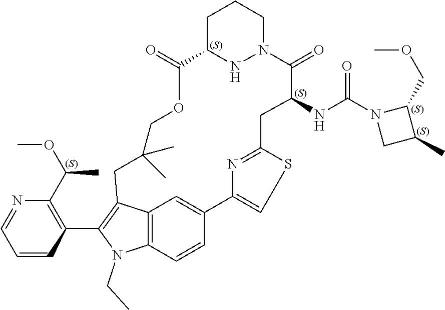 |
| DA270 | 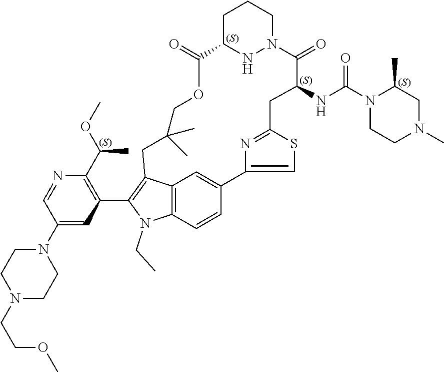 |
| DA271 | 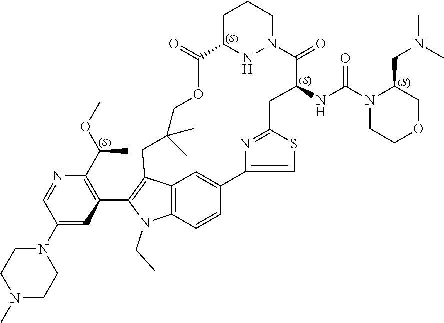 |
| DA272 | 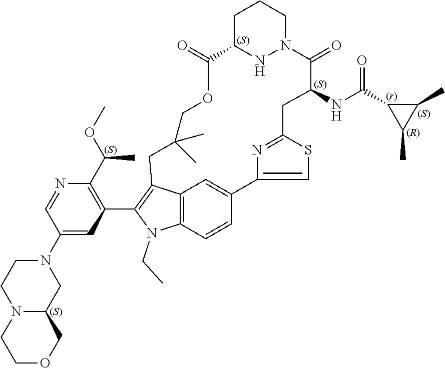 |
| DA273 | 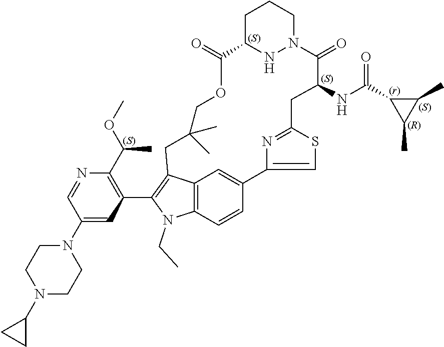 |
| DA274 | 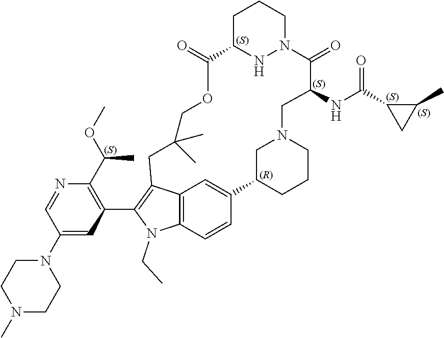 |
| DA275 | 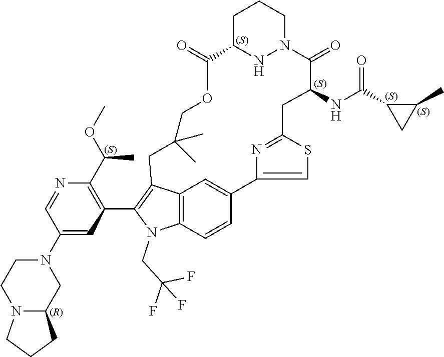 |
| DA276 | 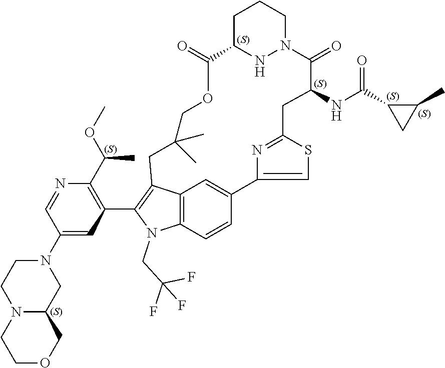 |
| DA277 | 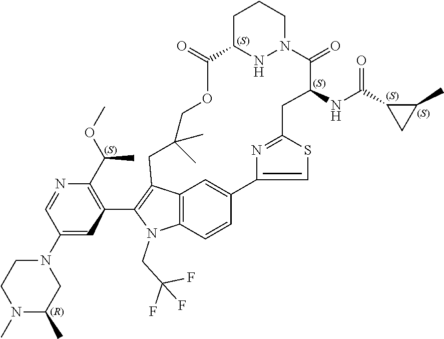 |
| DA278 | 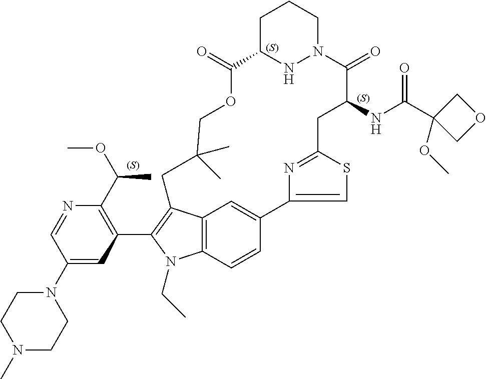 |
| DA279 | 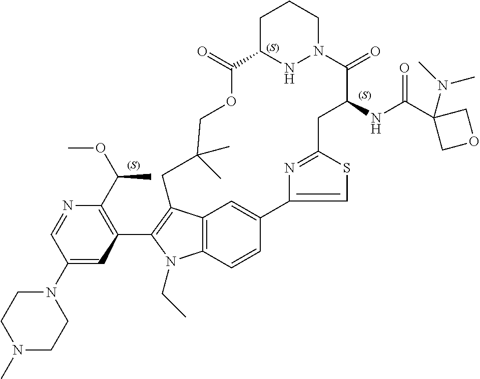 |
| DA280 | 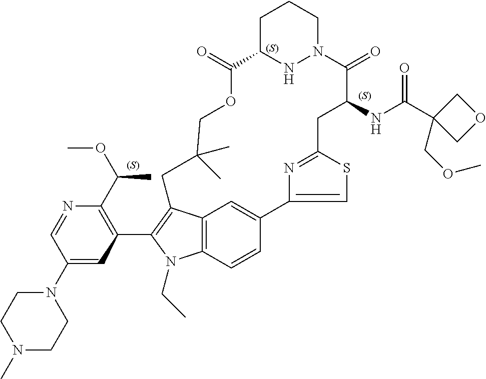 |
| DA281 | 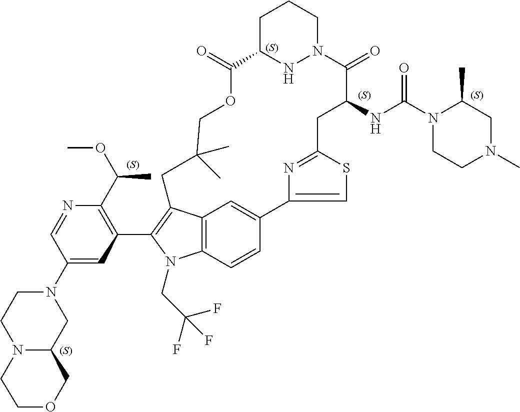 |
| DA282 | 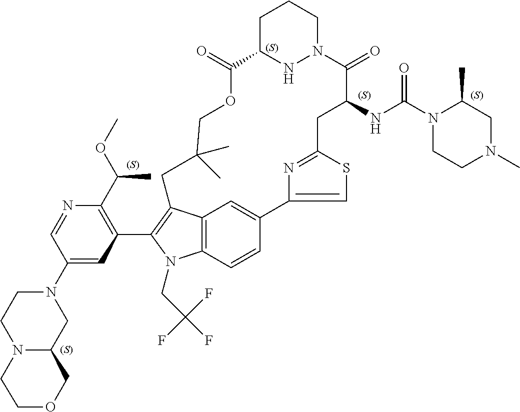 |
| DA283 | 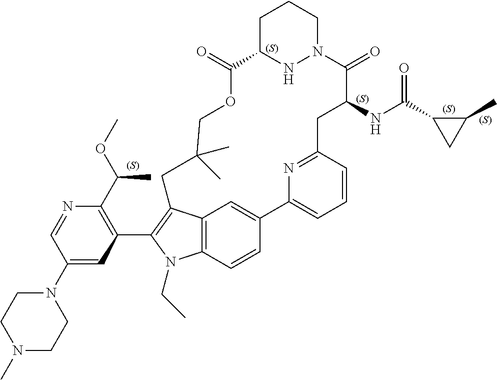 |
| DA284 | 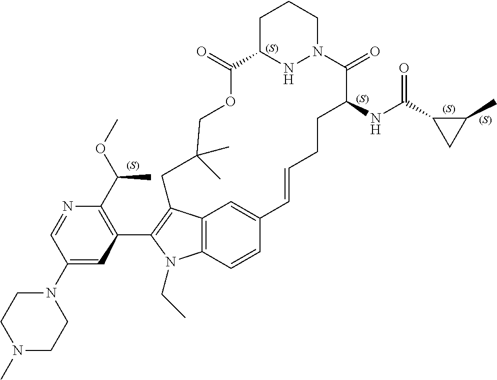 |
| DA285 | 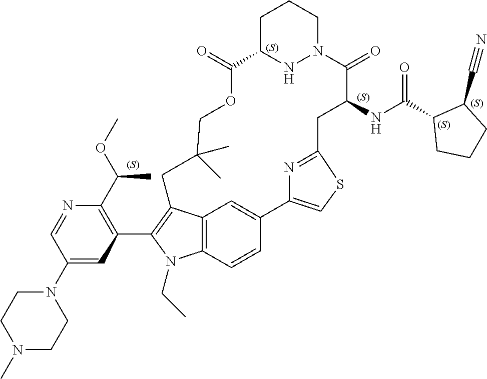 |
| DA286 | 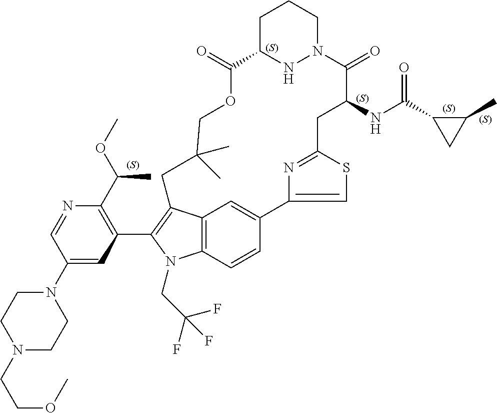 |
| DA287 | 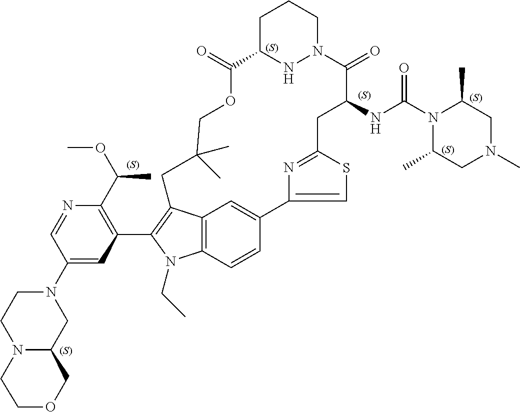 |
| DA288 | 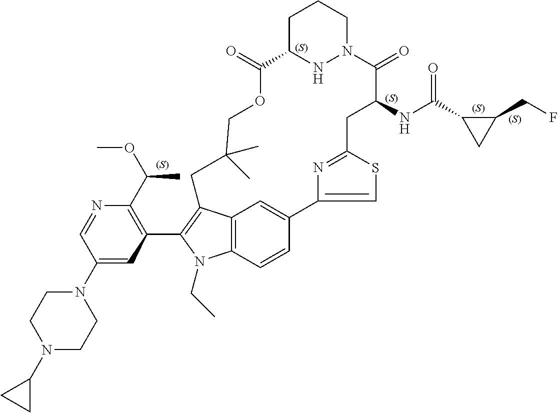 |
| DA289 | 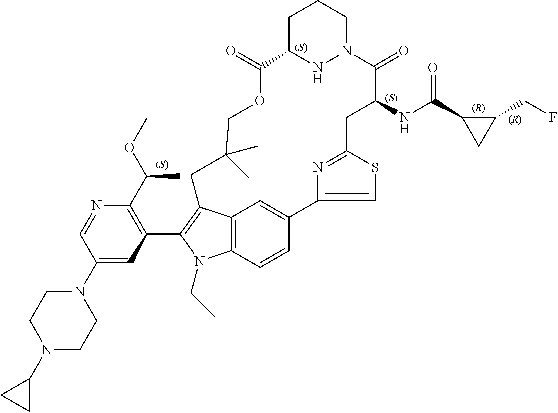 |
| DA290 | 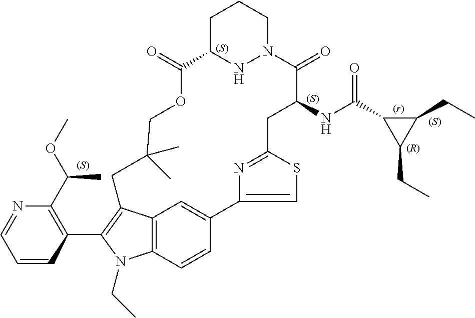 |
| DA291 | 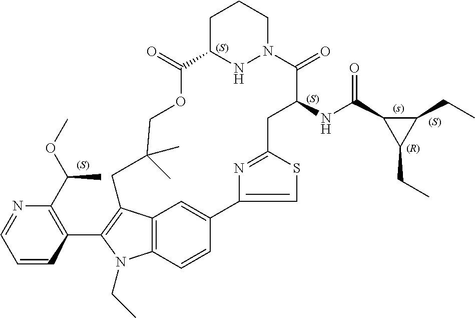 |
| DA292 | 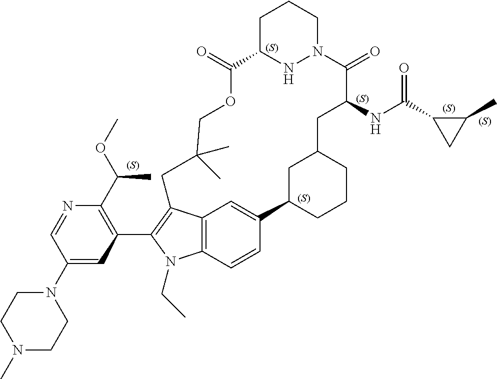 |
| DA293 | 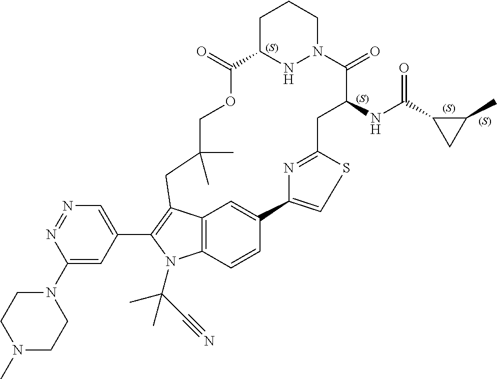 |
| DA294 | 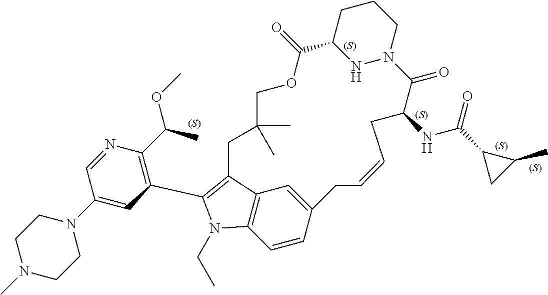 |
| DA295 | 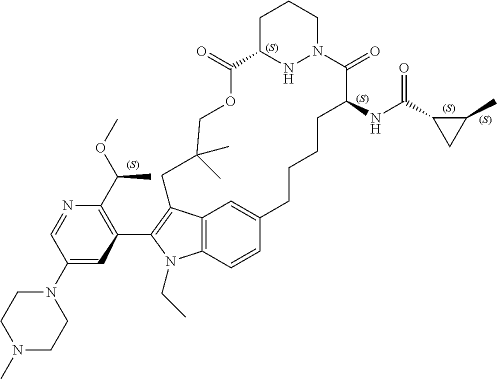 |
| DA296 | 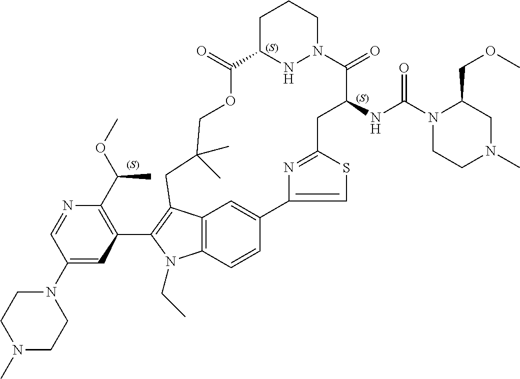 |
| DA297 | 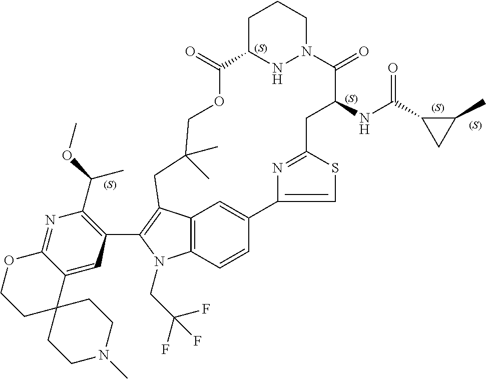 |
| DA298 | 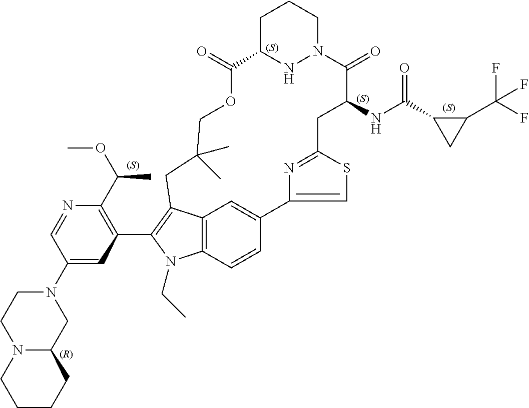 |
| DA299 | 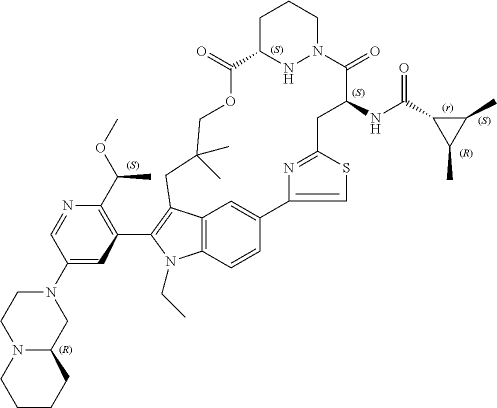 |
| DA300 | 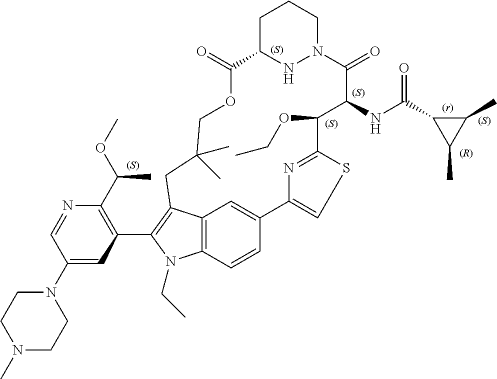 |
| DA301 | 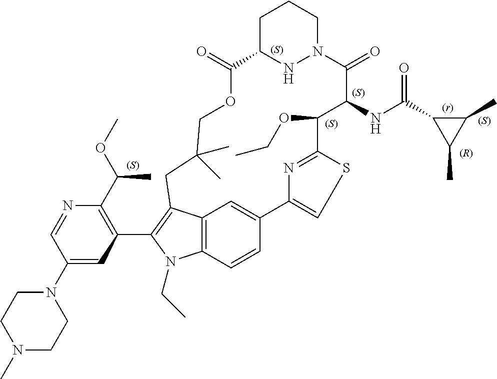 |
| DA302 | 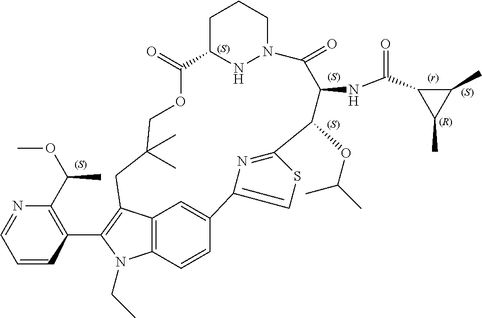 |
| DA303 | 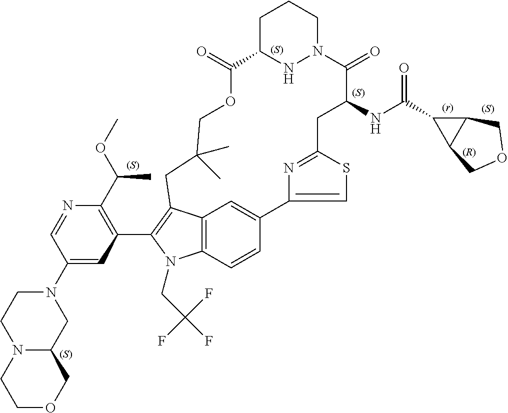 |
| DA304 | 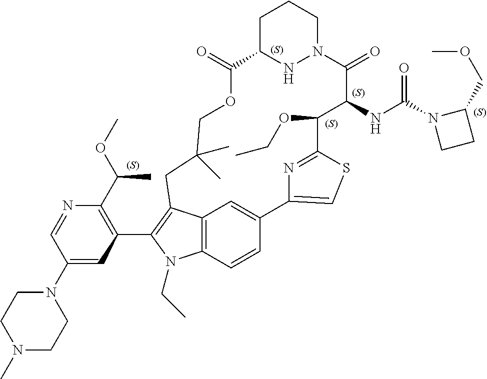 |
| DA305 | 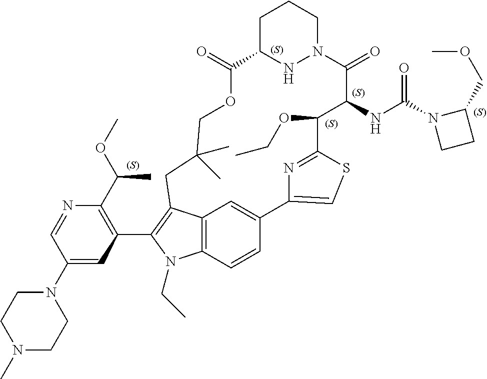 |
| DA306 | 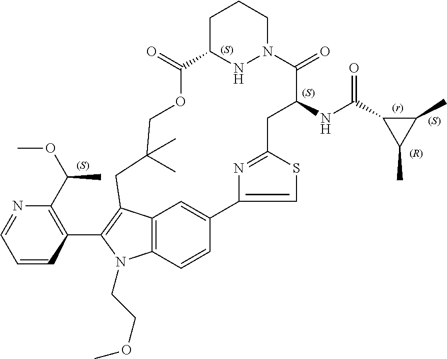 |
| DA307 | 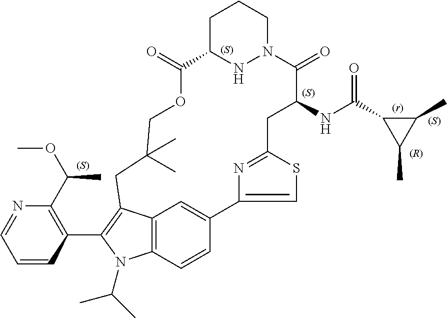 |
| DA308 | 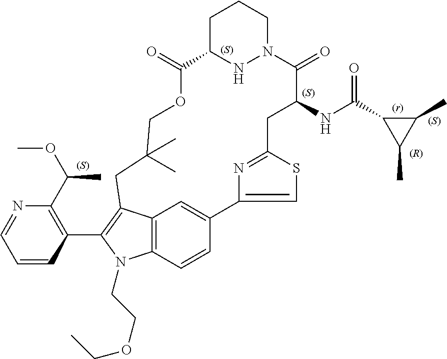 |
| DA309 | 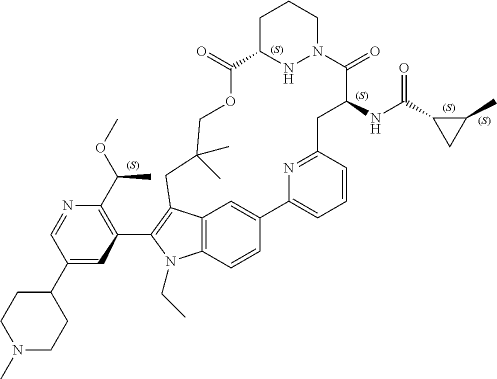 |
| DA310 | 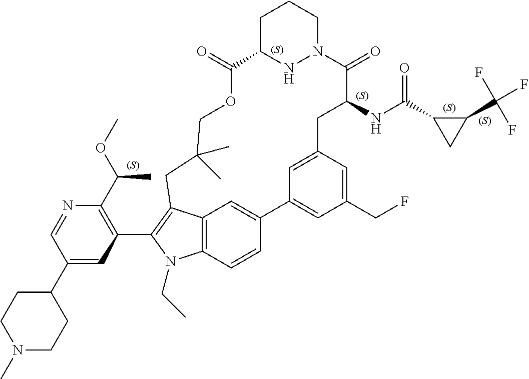 |
| DA311 | 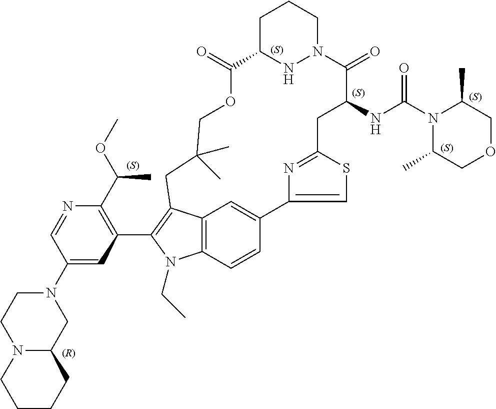 |
| DA312 | 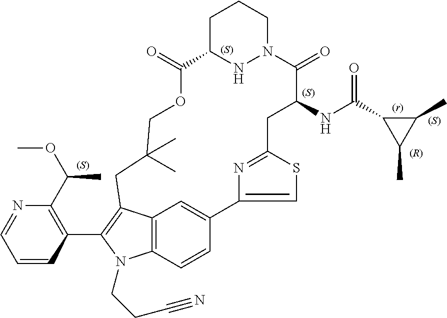 |
| DA313 | 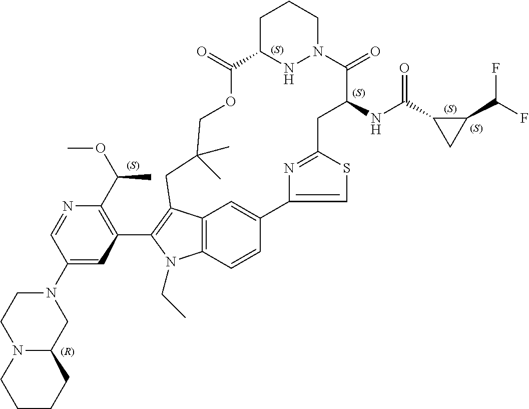 |
| DA314 | 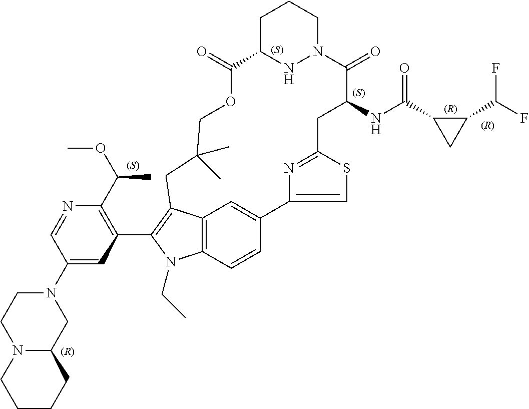 |
| DA315 | 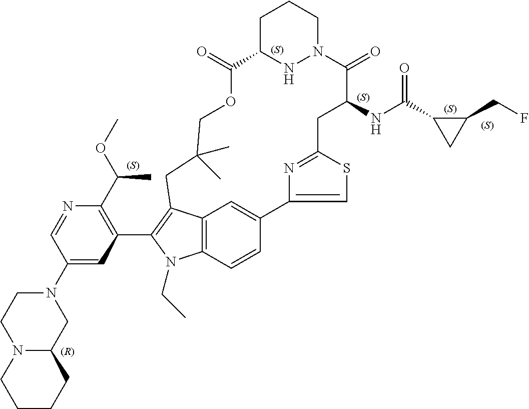 |
| DA316 | 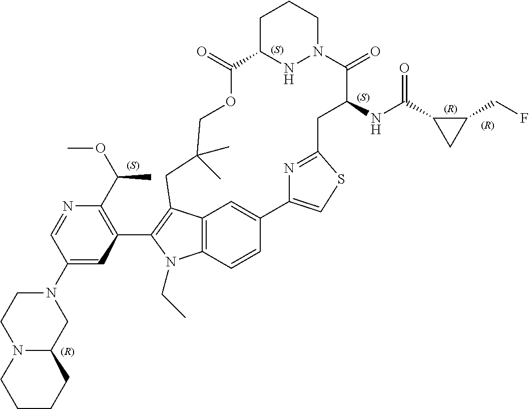 |
| DA317 | 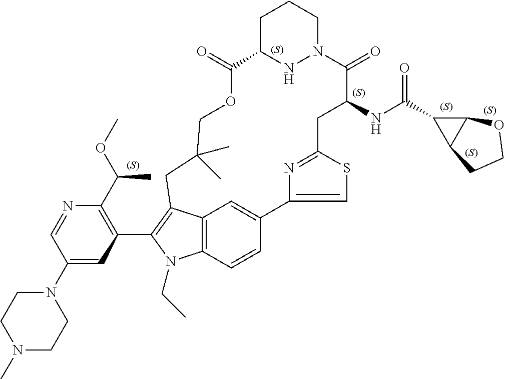 |
| DA318 | 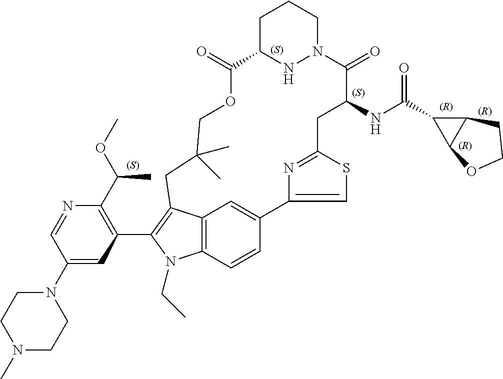 |
| DA319 | 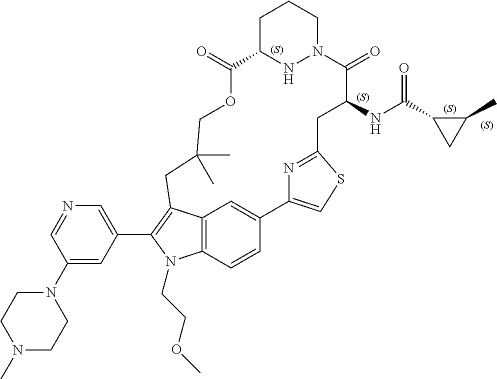 |
| DA320 | 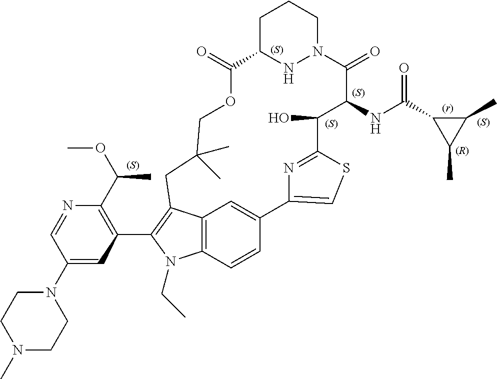 |
| DA321 | 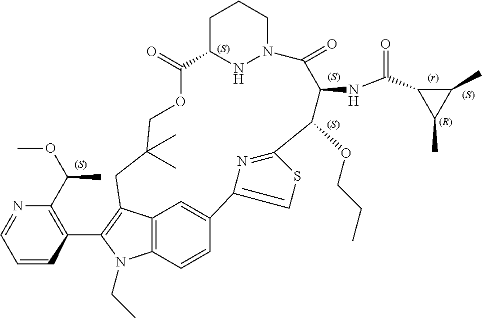 |
| DA322 | 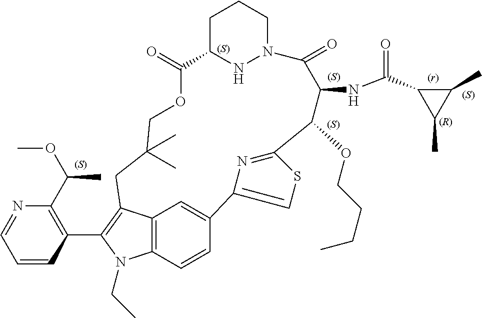 |
| DA323 | 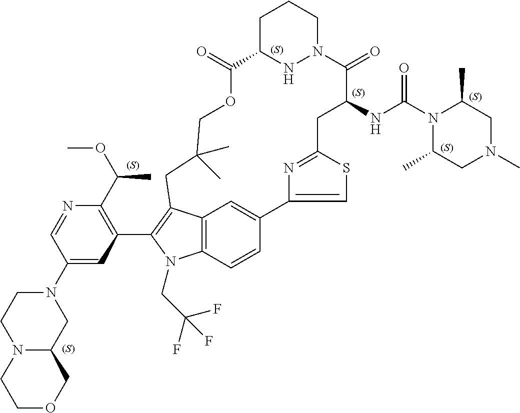 |
| DA324 | 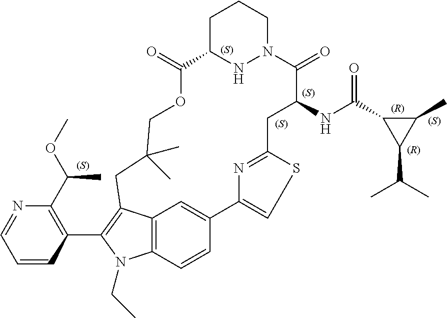 |
| DA325 | 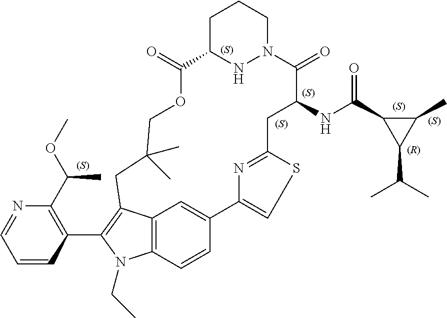 |
| DA326 | 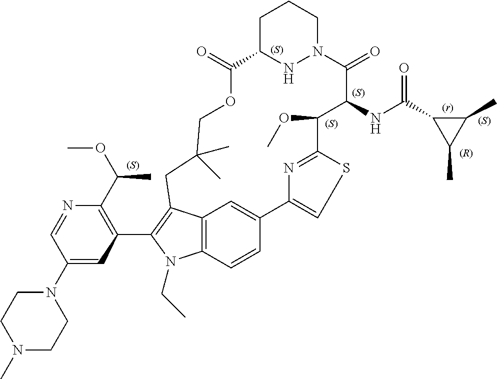 |
| DA327 | 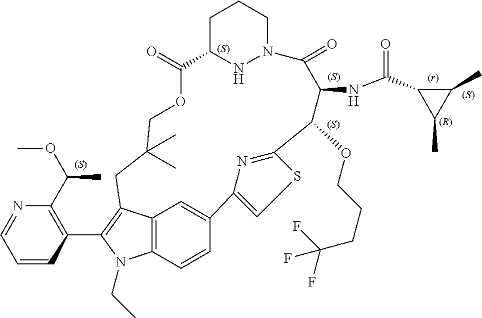 |
| DA328 | 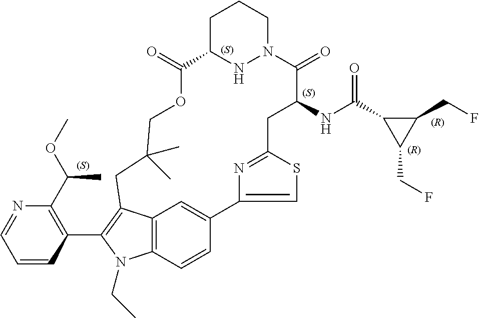 |
| DA329 | 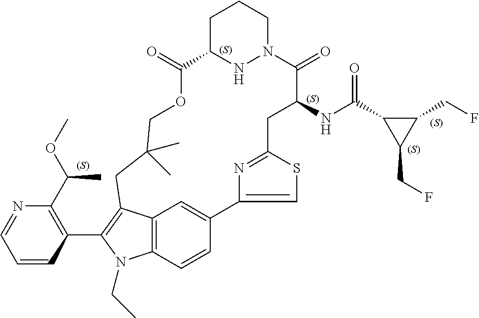 |
| DA330 | 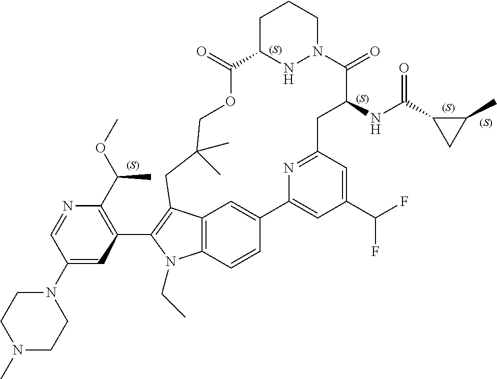 |
| DA331 | 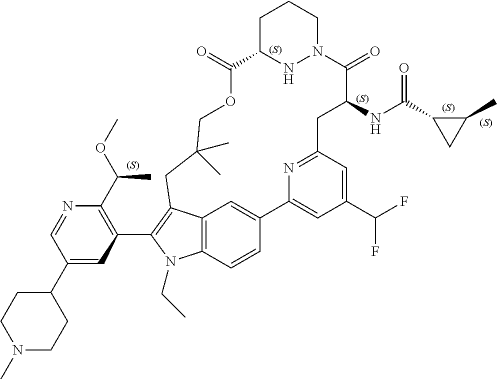 |
| DA332 | 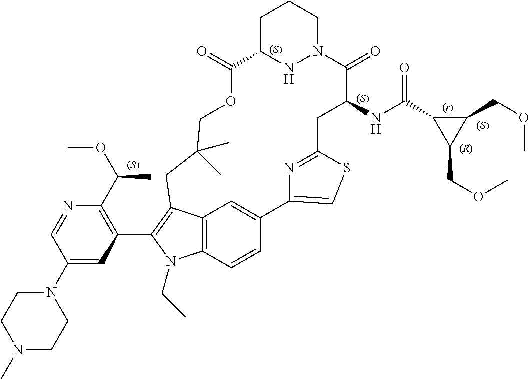 |
| DA333 | 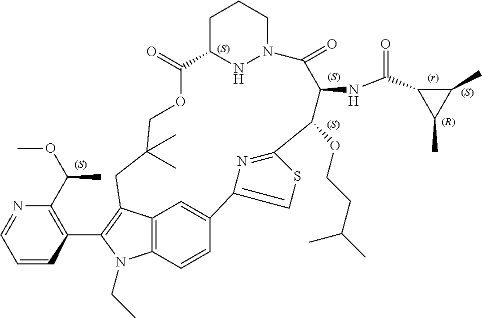 |
| DA334 | 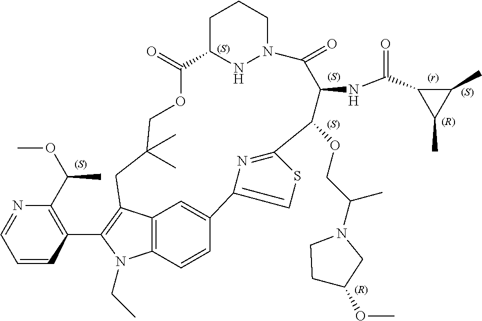 |
| DA335 | 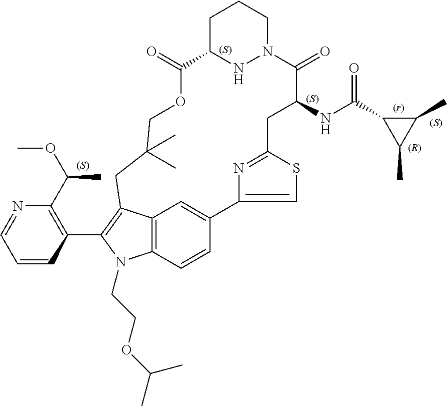 |
| DA336 | 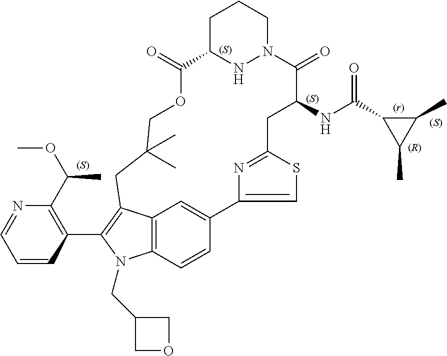 |
| DA337 | 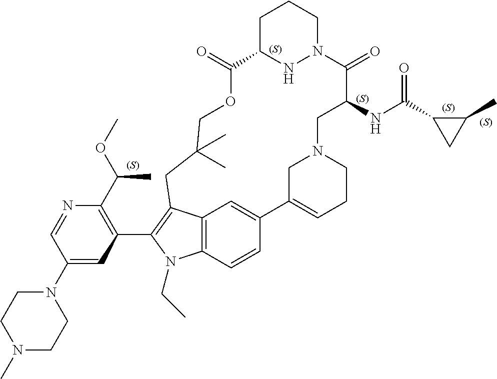 |
| DA338 | 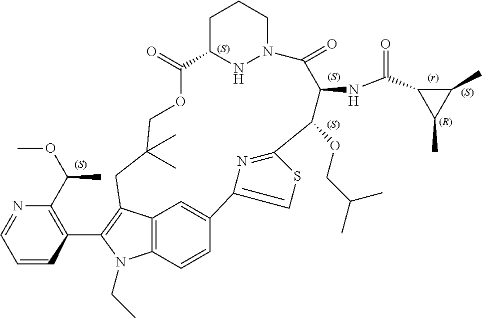 |
| DA339 | 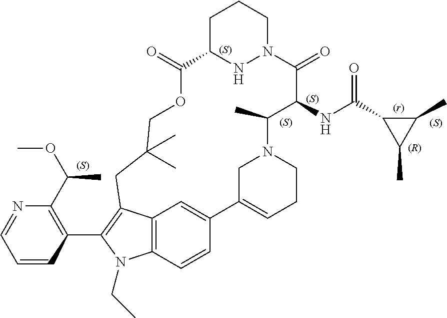 |
| DA340 | 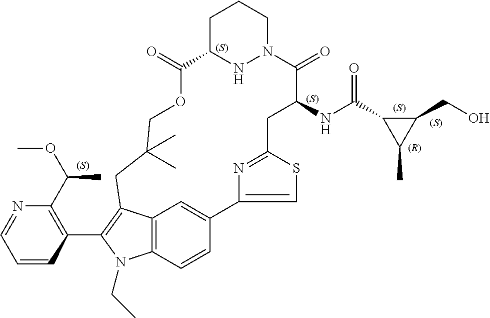 |
| DA341 | 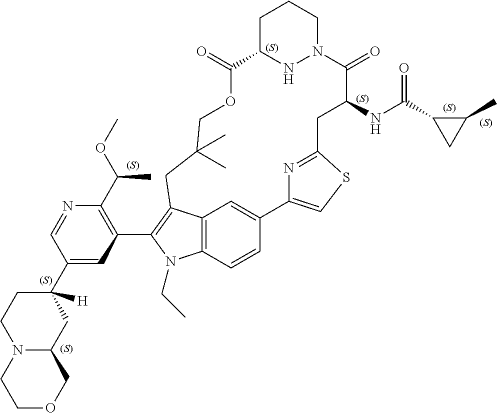 |
| DA342 | 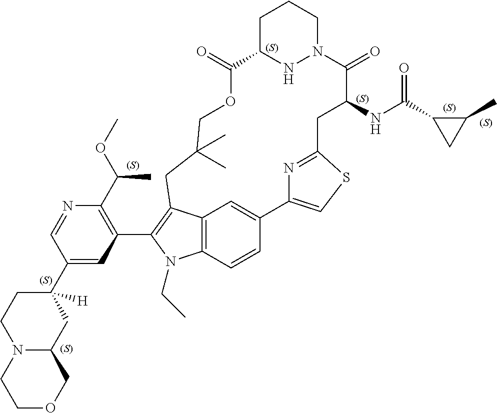 |
| DA343 | 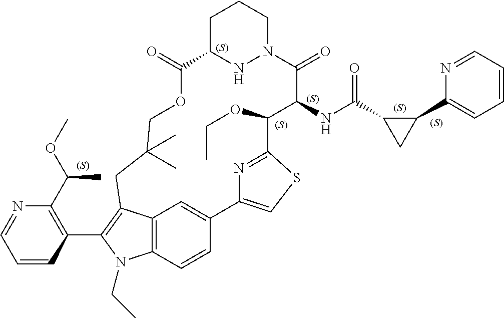 |
| DA344 | 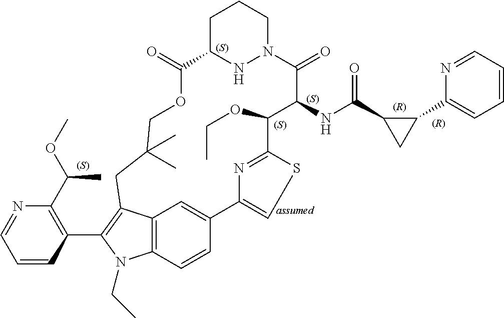 |
| DA345 | 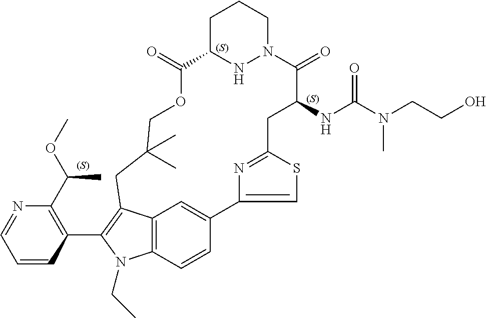 |
| DA346 | 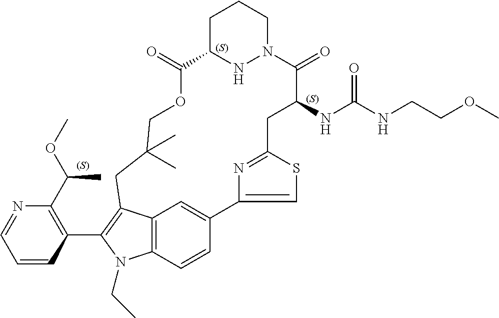 |
| DA347 | 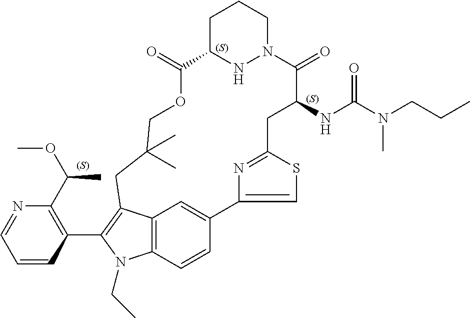 |
| DA348 | 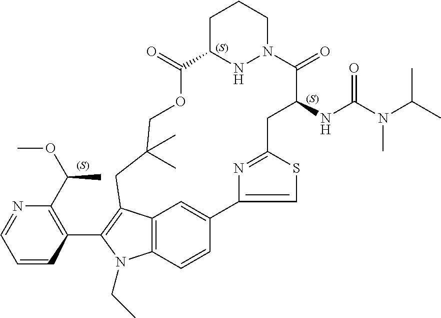 |
| DA349 | 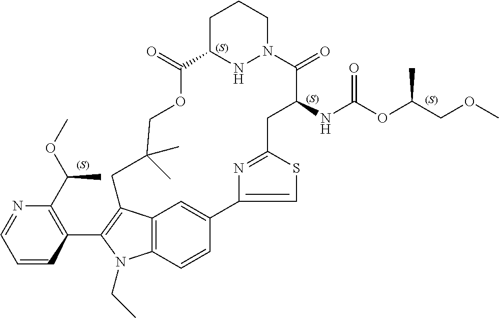 |
| DA350 | 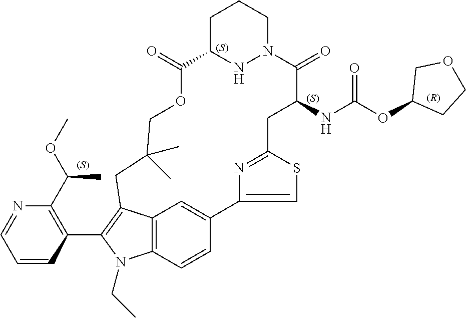 |
| DA351 | 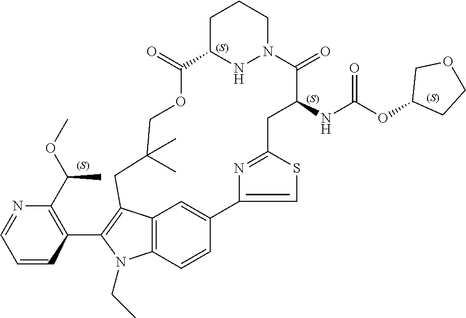 |
| DA352 | 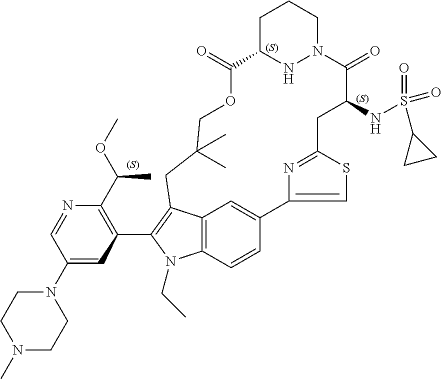 |

Note
that some compounds are shown with bonds as flat or wedged. In some instances, the relative stereochemistry of
stereoisomers has been determined; in some instances, the absolute stereochemistry has been determined. All
stereoisomers of the compounds of the foregoing table are contemplated by the present invention. In particular
embodiments, an atropisomer of a compound of the foregoing table is contemplated. Any compound shown in
brackets indicates that the compound is a diasteromer, and the absolute stereochemistry of such diastereomer may
not be known.

In some embodiments, the RAS(ON) inhibitor is selected from Table D1b, or a pharmaceutically acceptable salt or stereoisomer thereof. In some embodiments, the RAS(ON) inhibitor is selected from Table D1b, or a pharmaceutically acceptable salt or atropisomer thereof.

TABLE D1b
Certain Compounds of the Present Invention

| Ex. # | Structure |
|---|---|
| DA354 | 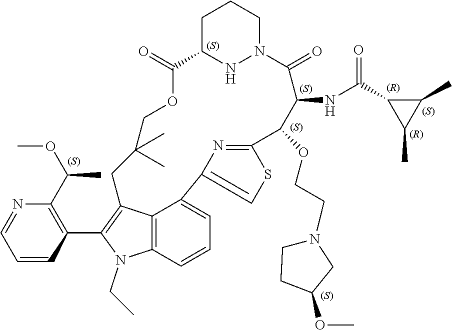 |
| DA355 | 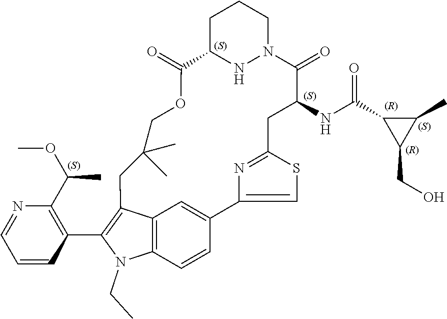 |
| DA356 | 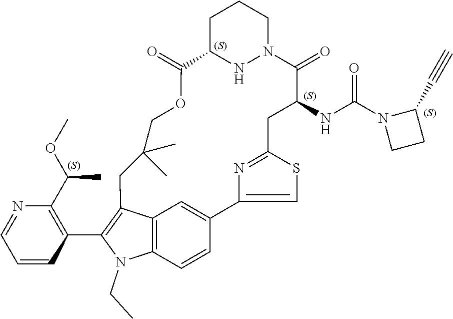 |
| DA357 | 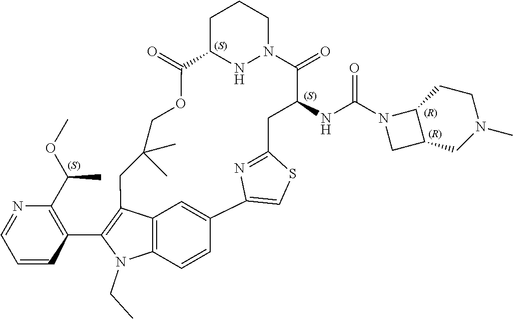 |
| DA358 | 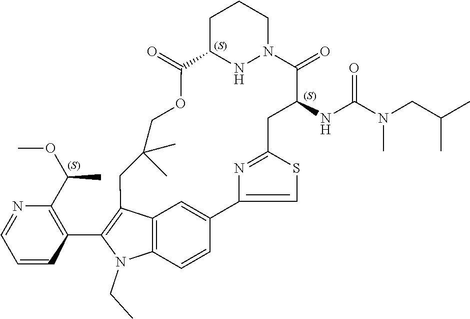 |
| DA359 | 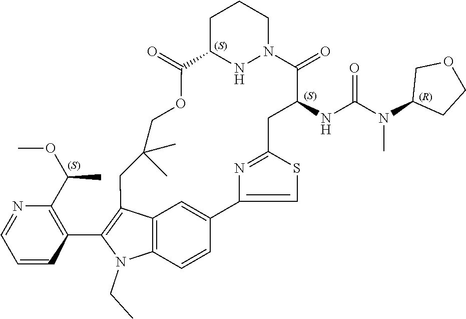 |
| DA360 | 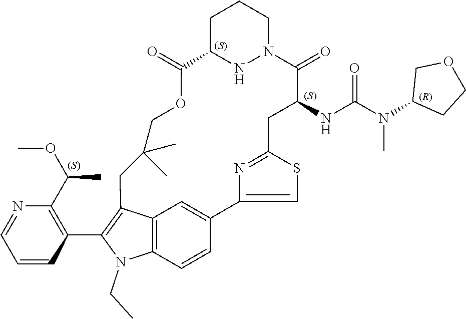 |
| DA361 | 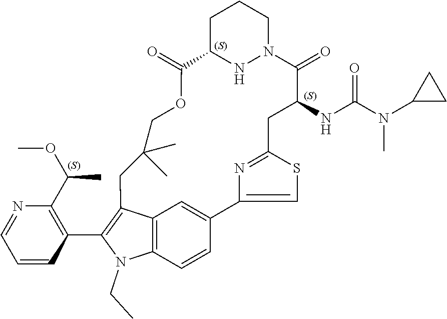 |
| DA362 | 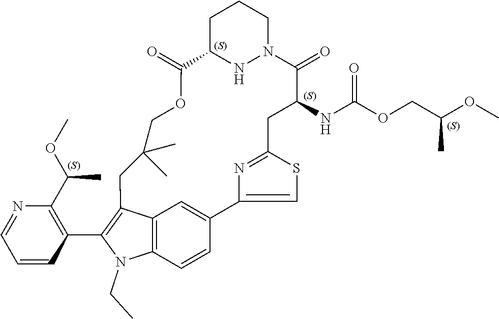 |
| DA363 | 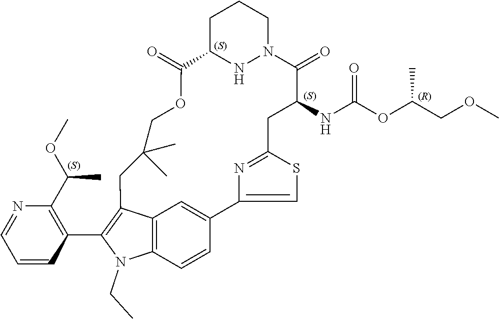 |
| DA364 | 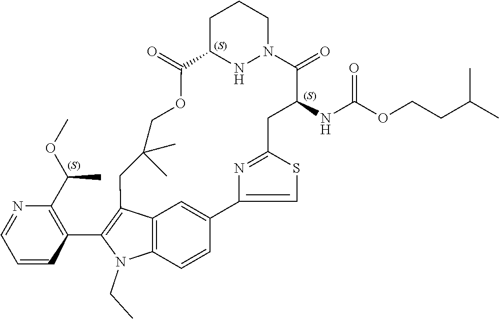 |
| DA365 | 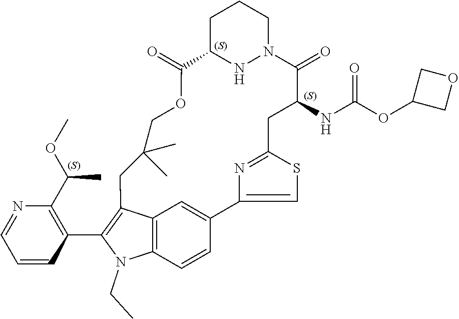 |
| DA366 | 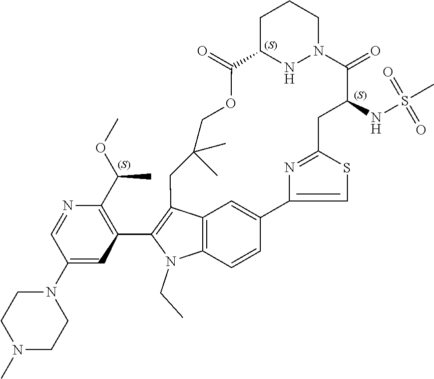 |
| DA367 | 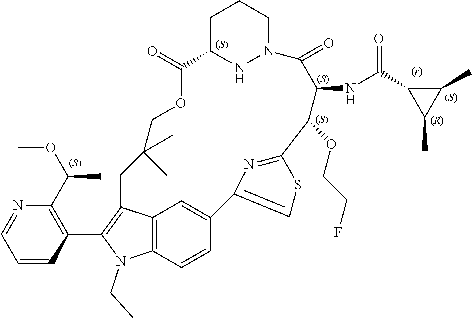 |
| DA368 | 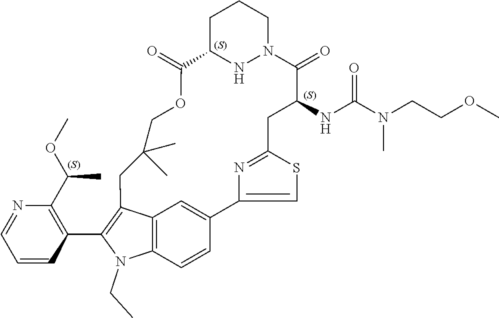 |
| DA369 | 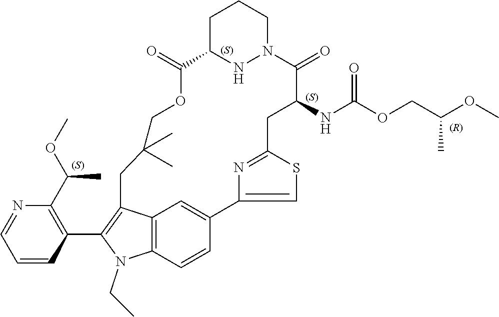 |
| DA370 | 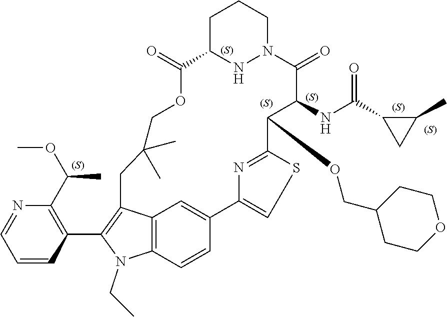 |
| DA371 | 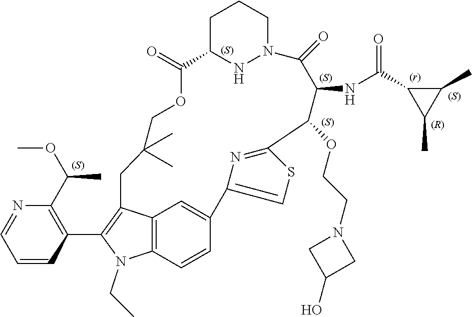 |
| DA372 | 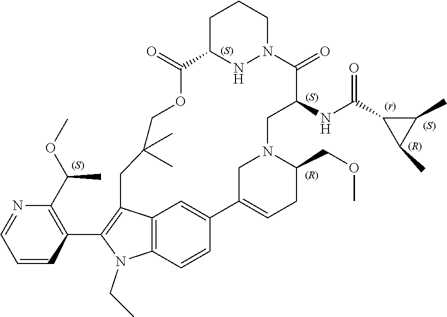 |
| DA373 | 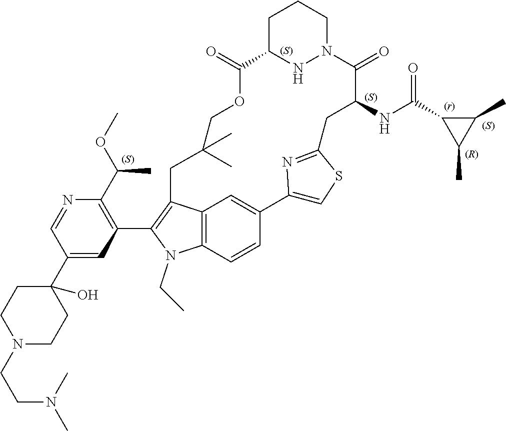 |
| DA374 | 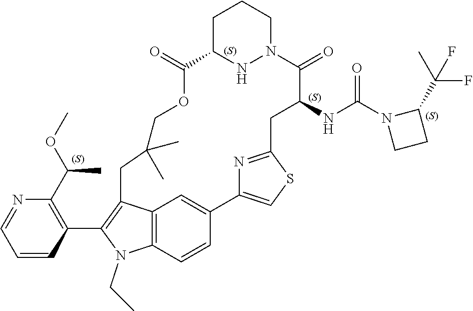 |
| DA375 | 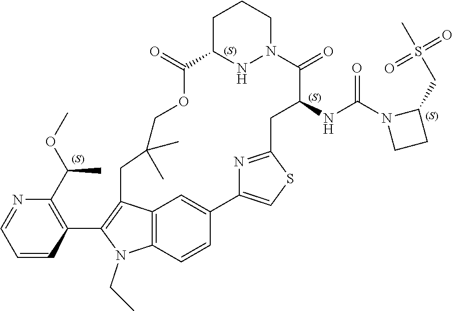 |
| DA376 | 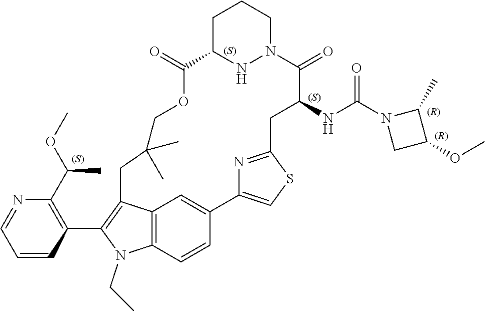 |
| DA377 | 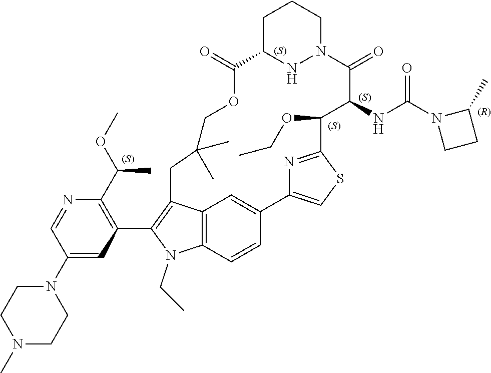 |
| DA378 | 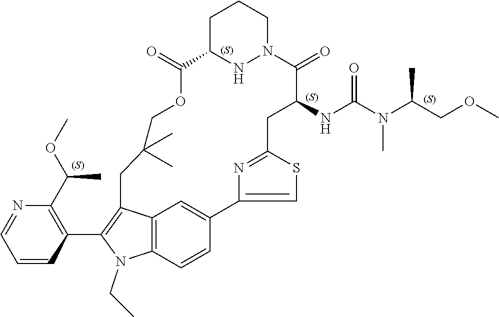 |
| DA379 | 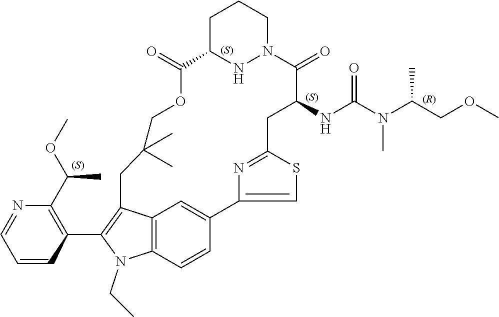 |
| DA380 | 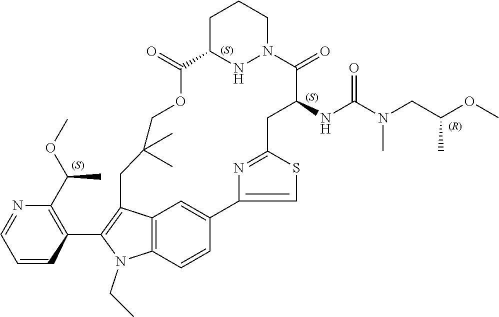 |
| DA381 | 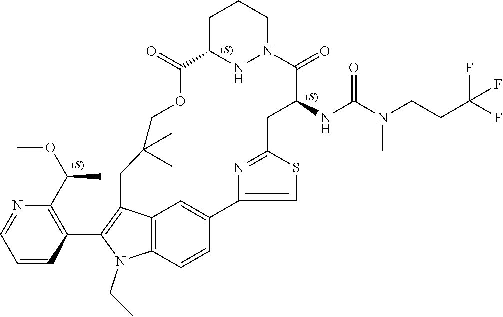 |
| DA382 | 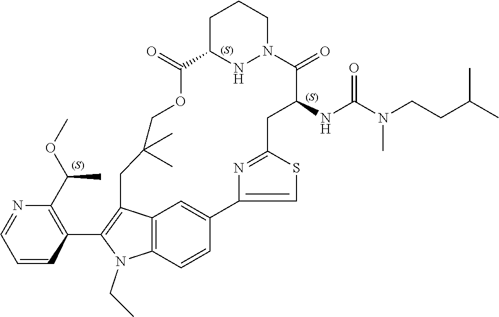 |
| DA383 | 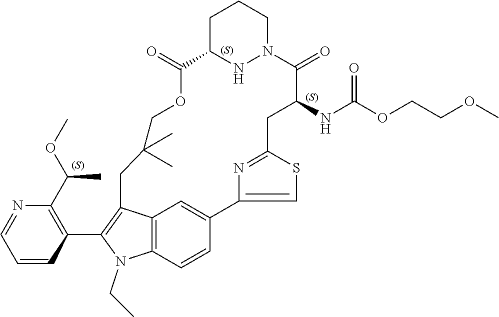 |
| DA384 | 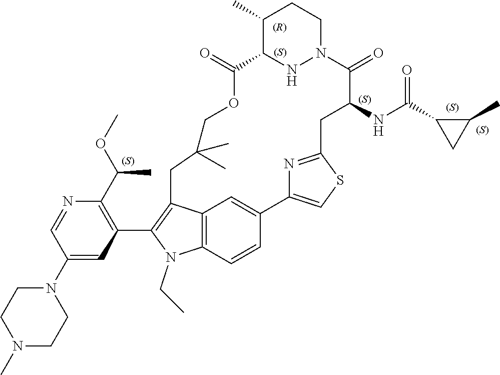 |
| DA385 | 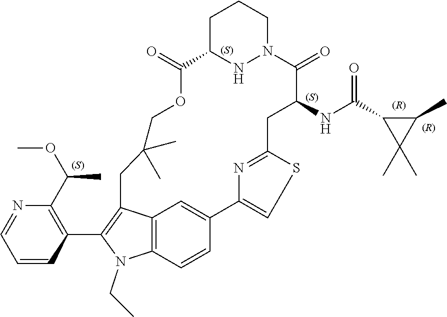 |
| DA386 | 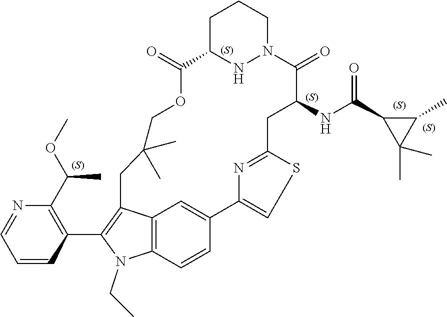 |
| DA387 | 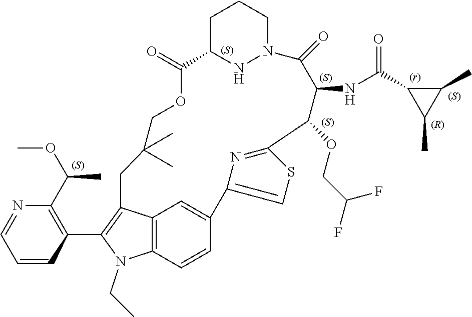 |
| DA388 | 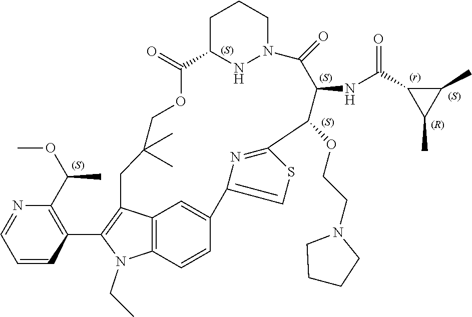 |
| DA389 | 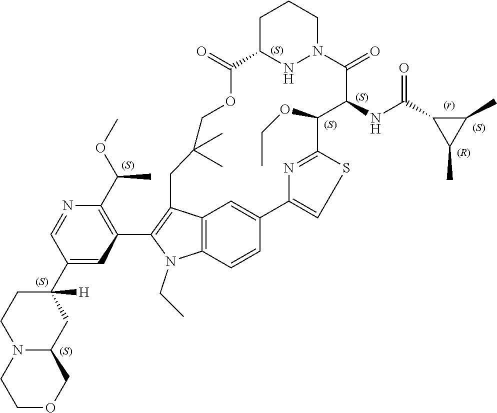 |
| DA390 | 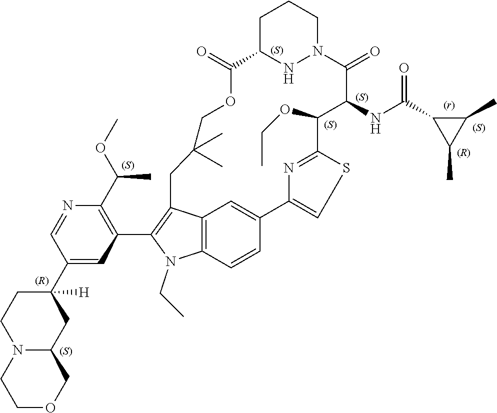 |
| DA391 | 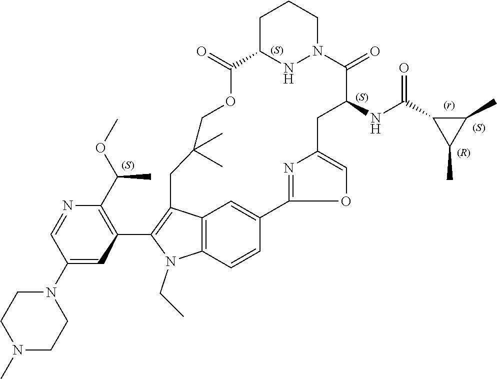 |
| DA392 | 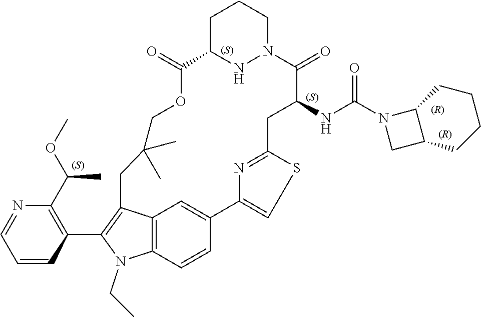 |
| DA393 | 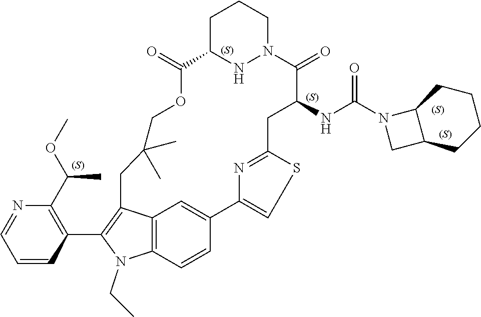 |
| DA394 | 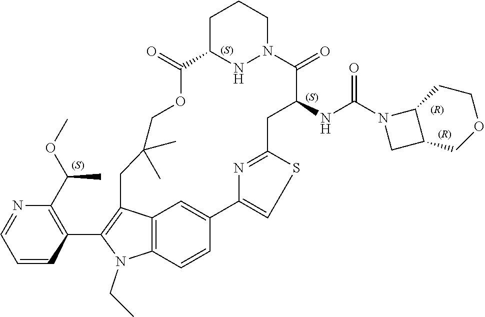 |
| DA395 | 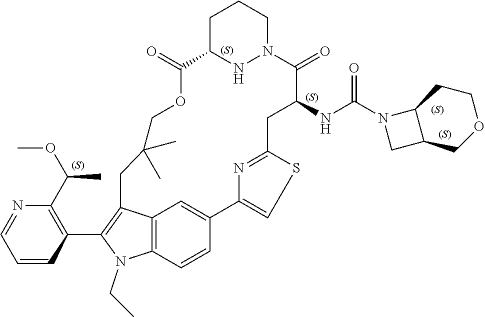 |
| DA396 | 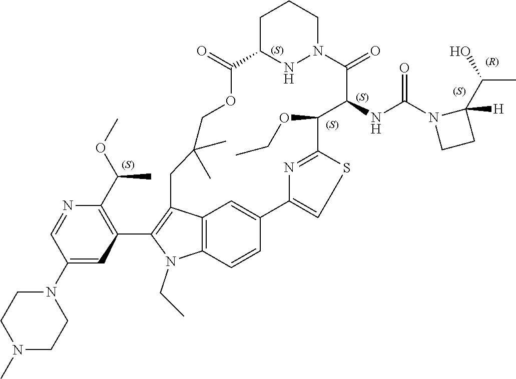 |
| DA397 | 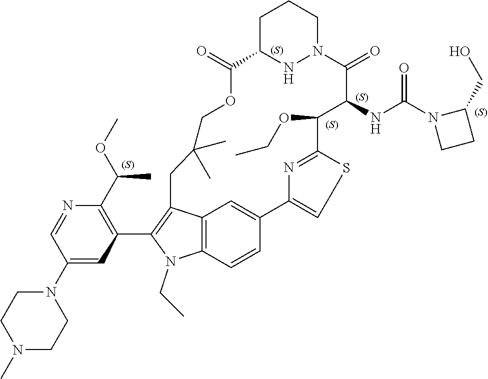 |
| DA398 | 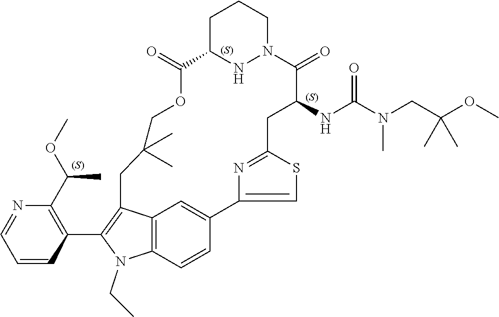 |
| DA399 | 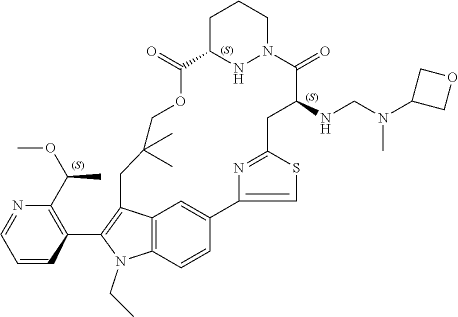 |
| DA400 | 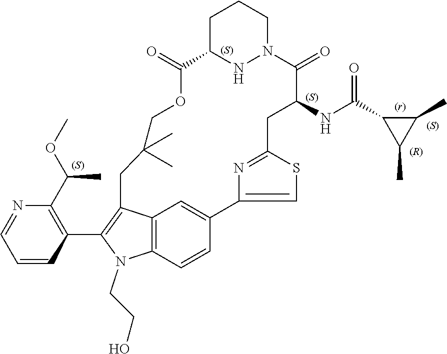 |
| DA401 | 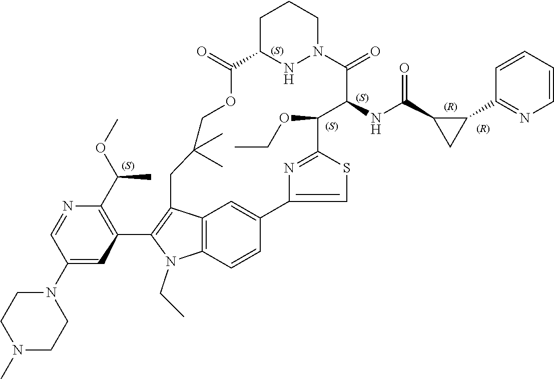 |
| DA402 | 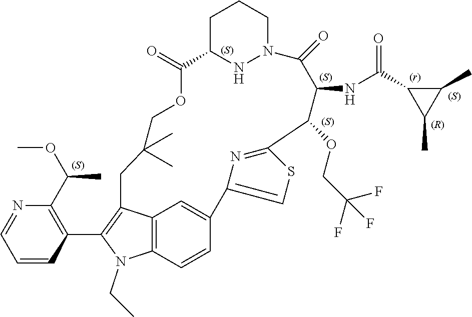 |
| DA403 | 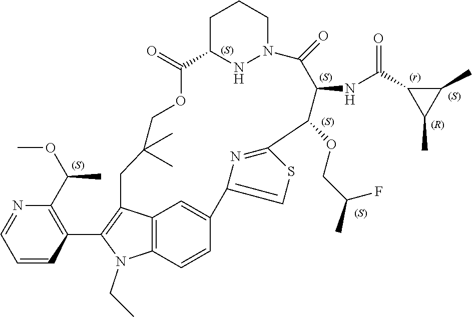 |
| DA404 | 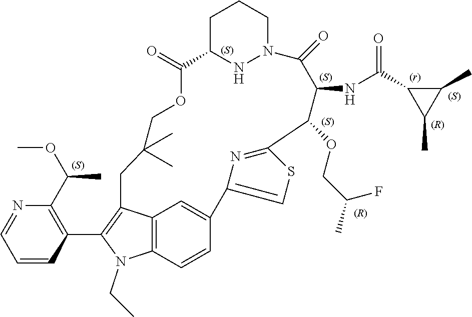 |
| DA405 | 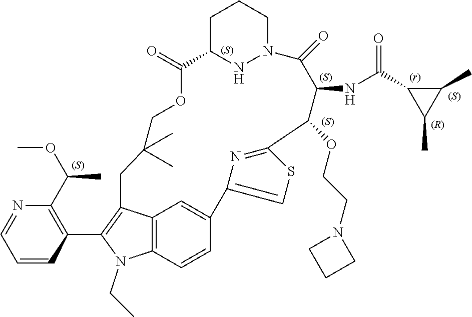 |
| DA406 | 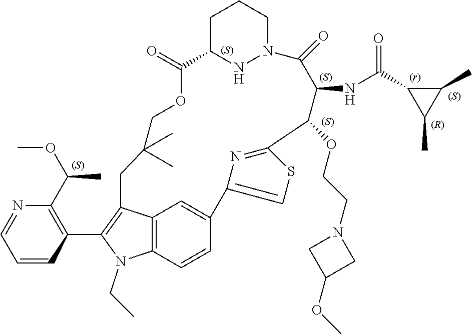 |
| DA407 | 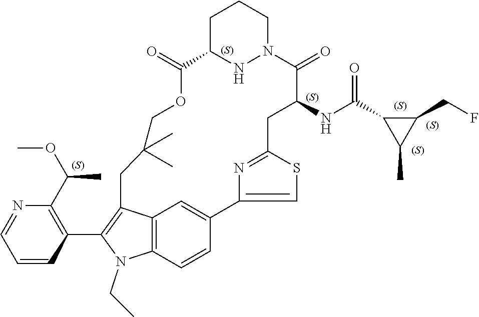 |
| DA408 | 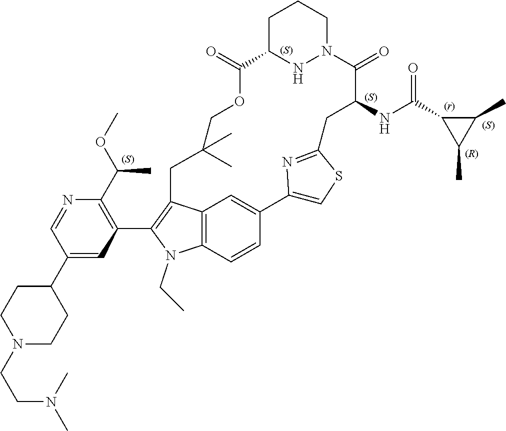 |
| DA409 | 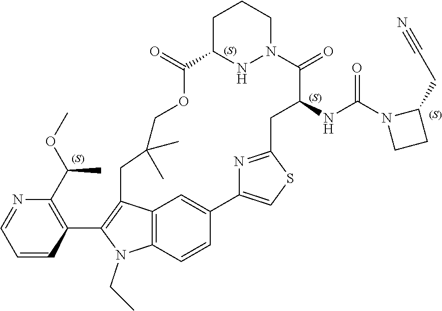 |
| DA410 | 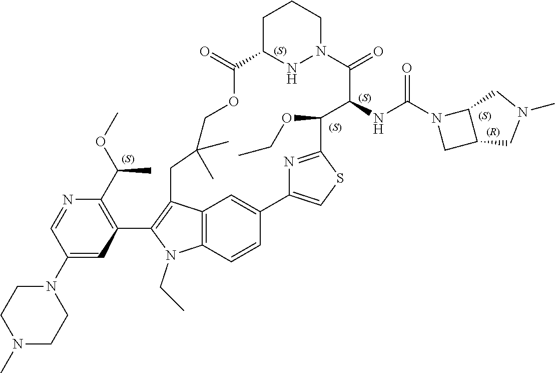 |
| DA411 | 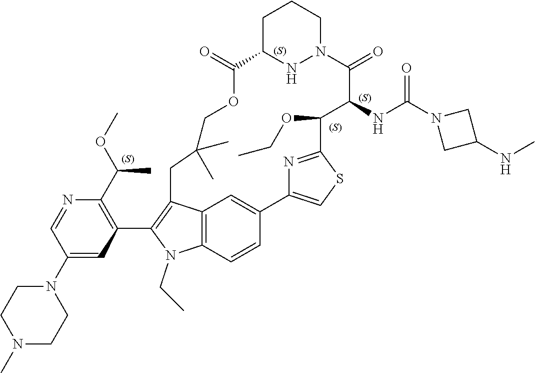 |
| DA412 | 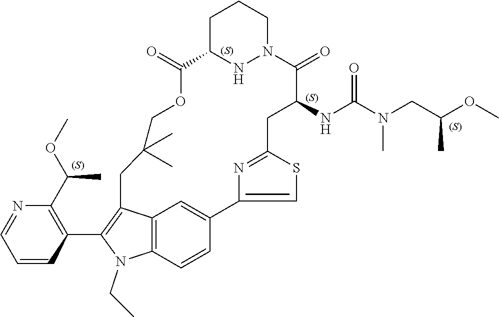 |
| DA413 | 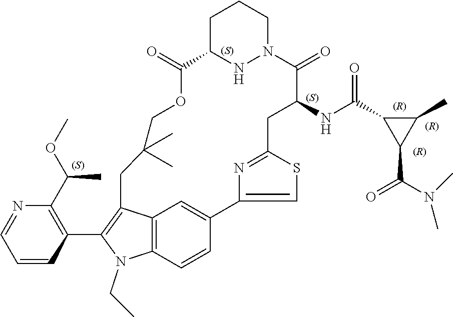 |
| DA414 | 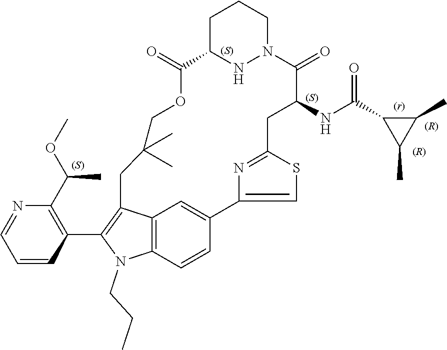 |
| DA415 | 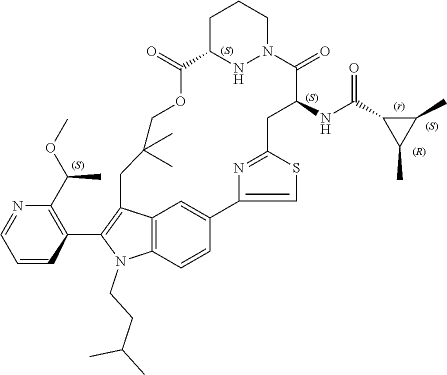 |
| DA416 | 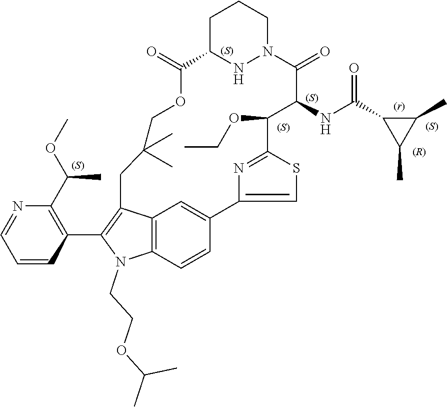 |
| DA417 | 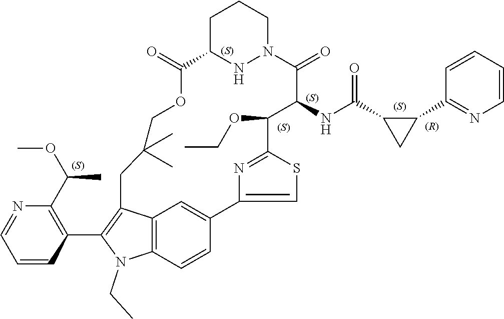 |
| DA418 | 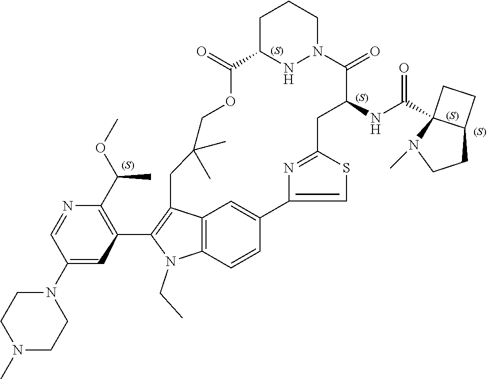 |
| DA419 | 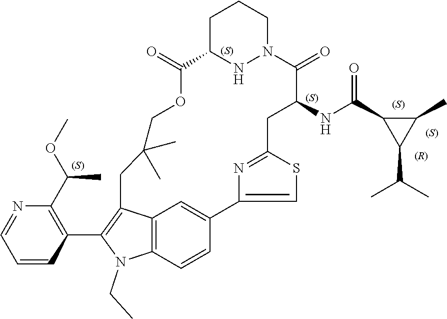 |
| DA420 | 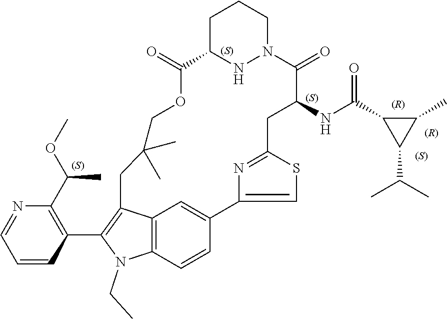 |
| DA421 | 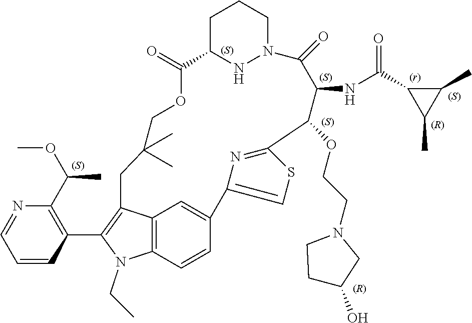 |
| DA422 | 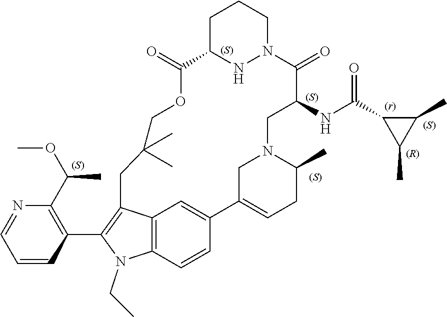 |
| DA423 | 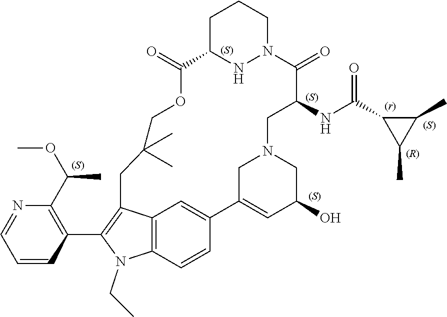 |
| DA424 | 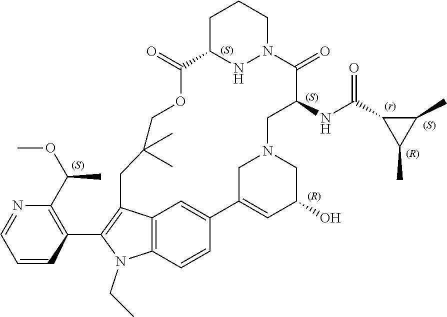 |
| DA425 | 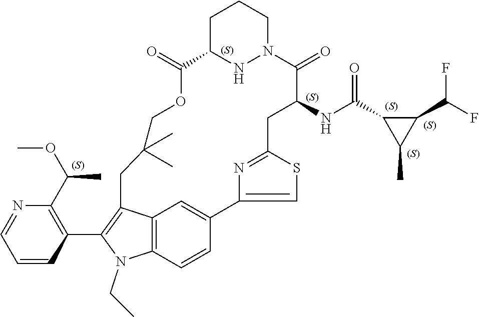 |
| DA426 | 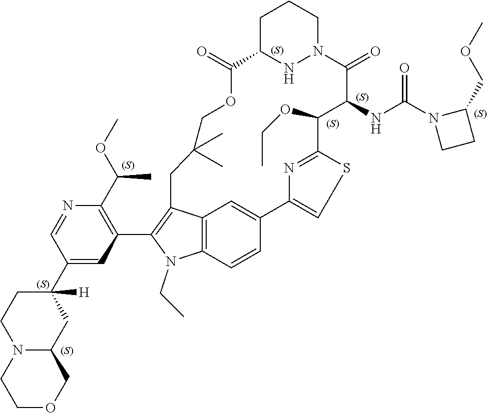 |
| DA427 | 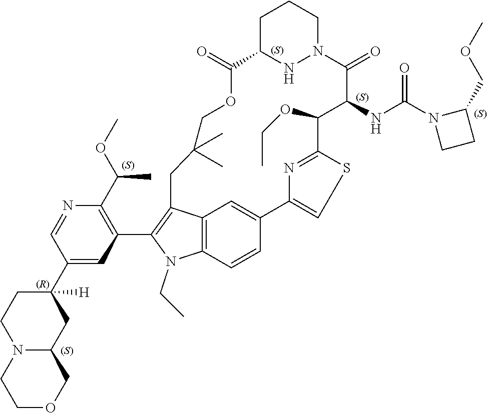 |
| DA428 | 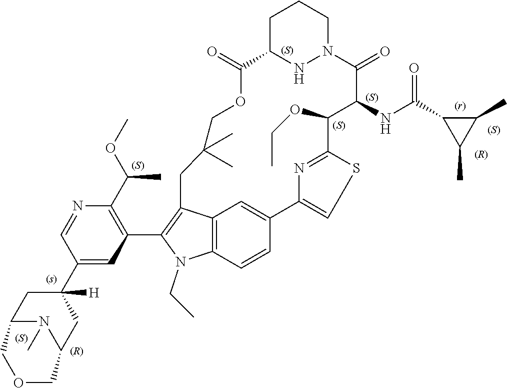 |
| DA429 | 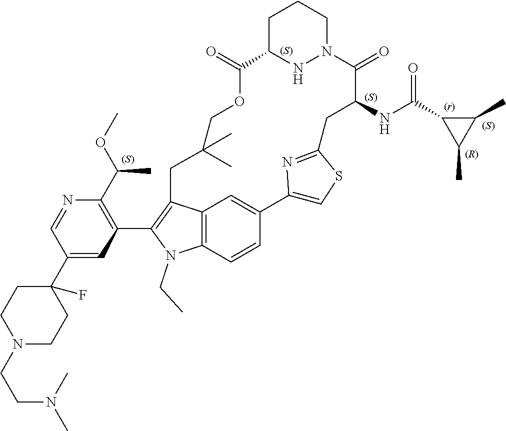 |
| DA430 | 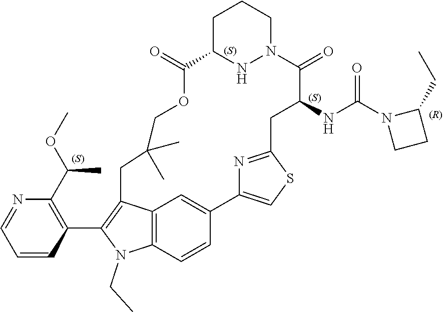 |
| DA431 | 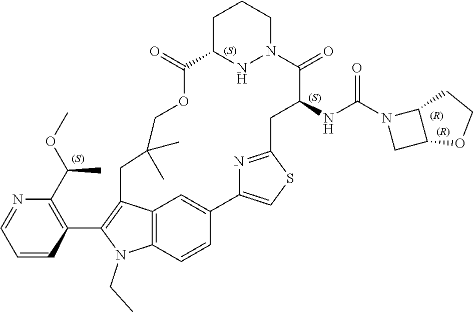 |
| DA432 | 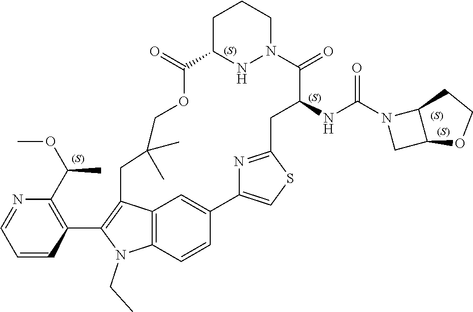 |
| DA433 | 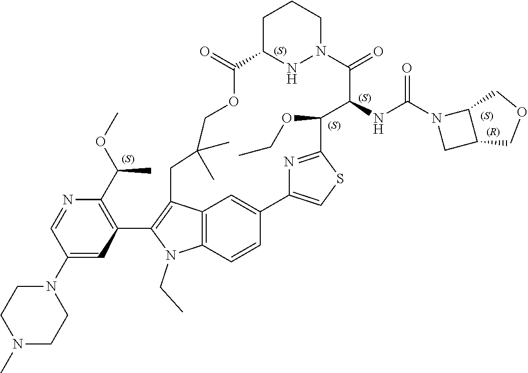 |
| DA434 | 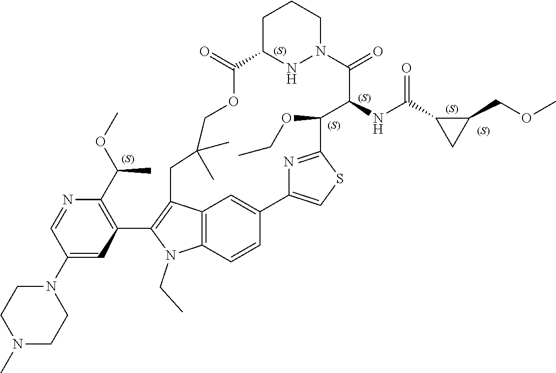 |
| DA435 | 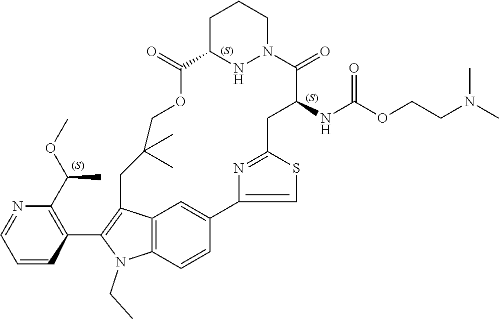 |
| DA436 | 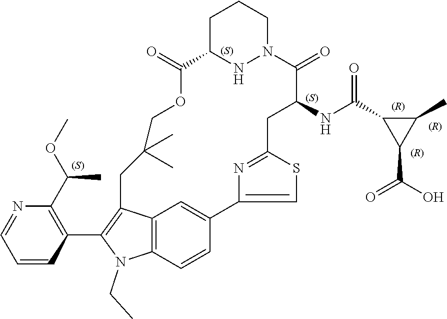 |
| DA437 | 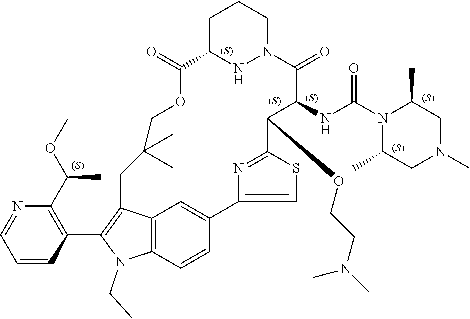 |
| DA438 | 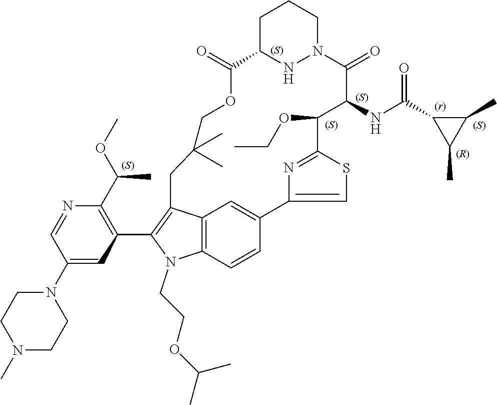 |
| DA439 | 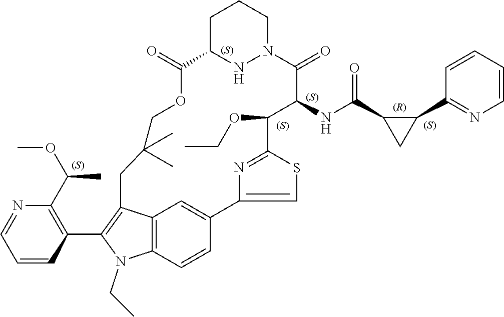 |
| DA440 | 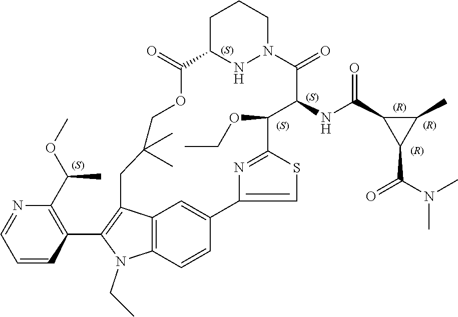 |
| DA441 | 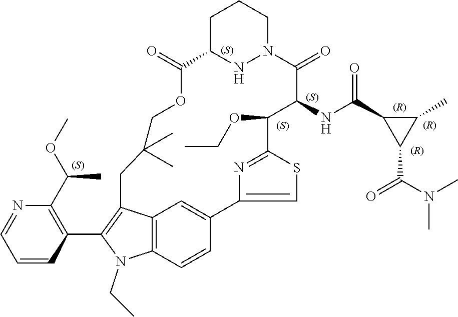 |
| DA442 | 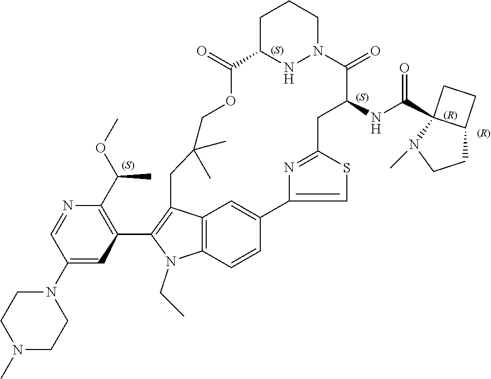 |
| DA443 | 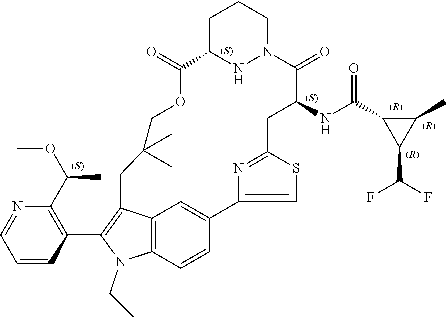 |
| DA444 | 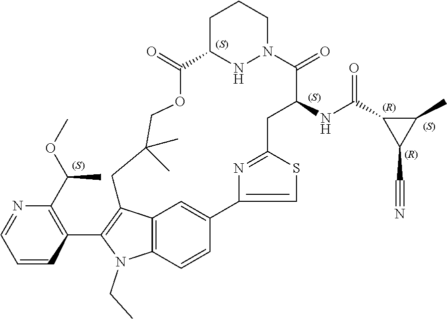 |
| DA445 | 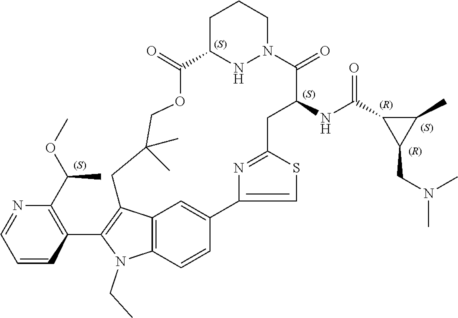 |
| DA446 | 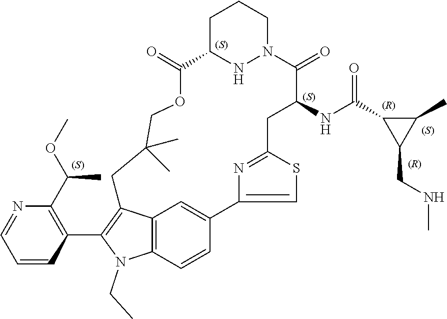 |
| DA447 | 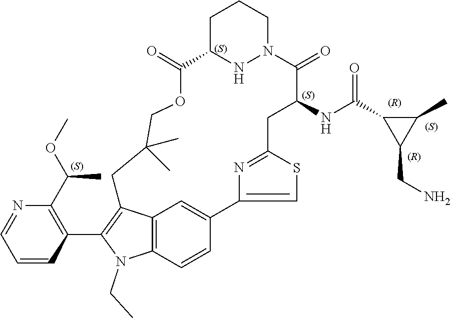 |
| DA448 | 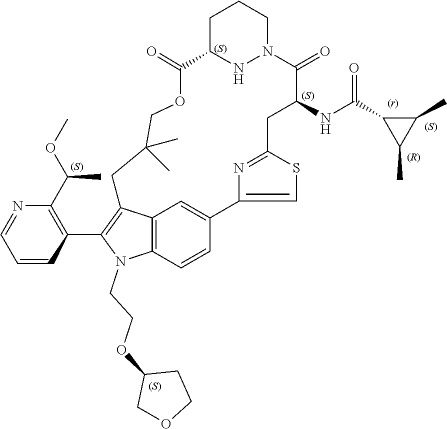 |
| DA449 | 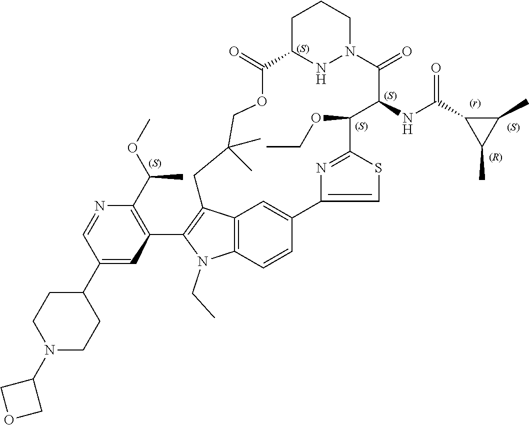 |
| DA450 | 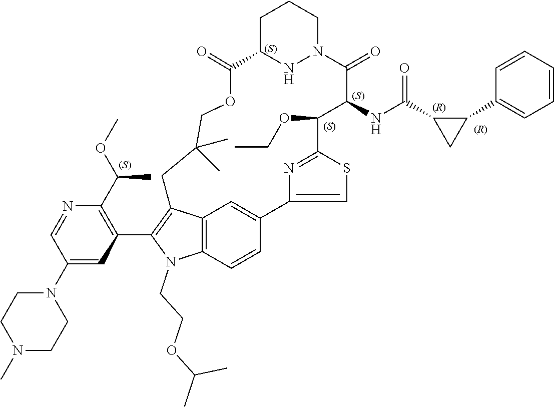 |
| DA451 | 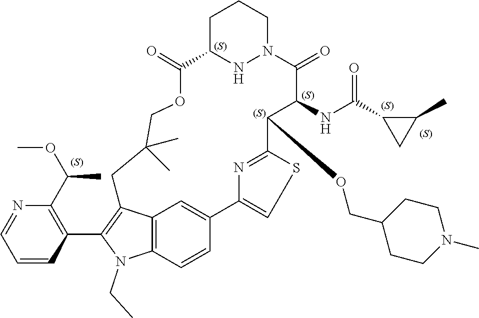 |
| DA452 | 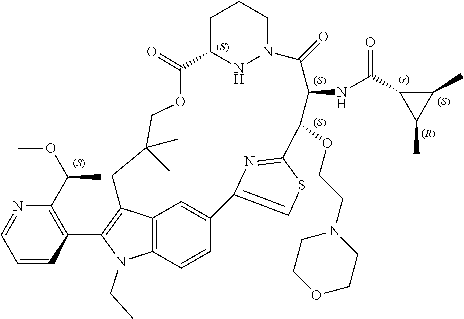 |
| DA453 | 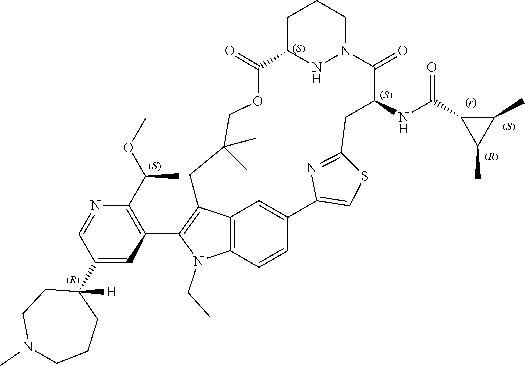 |
| DA454 | 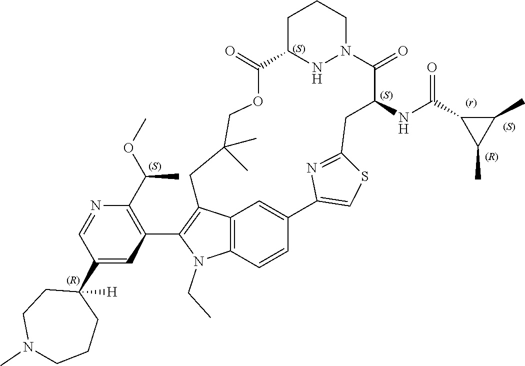 |
| DA455 | 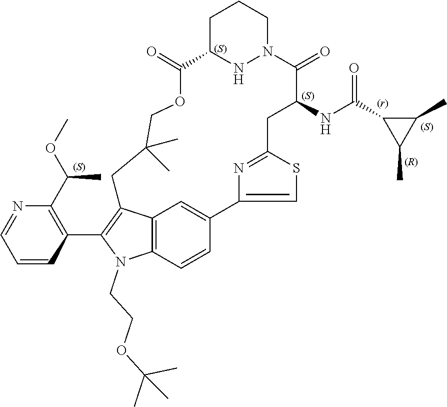 |
| DA456 | 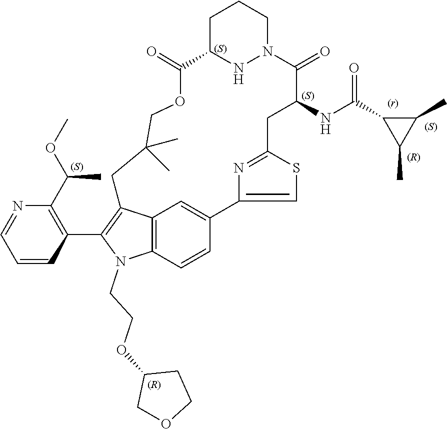 |
| DA457 | 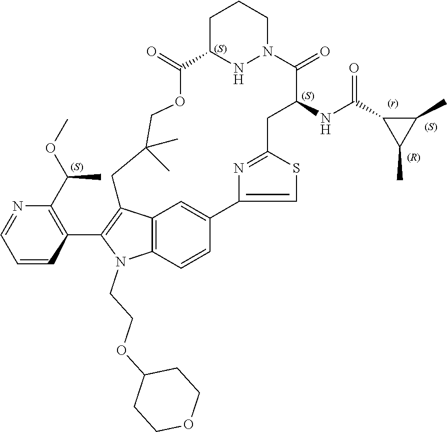 |
| DA458 | 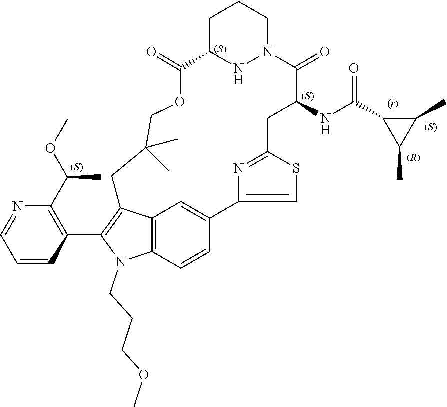 |
| DA459 | 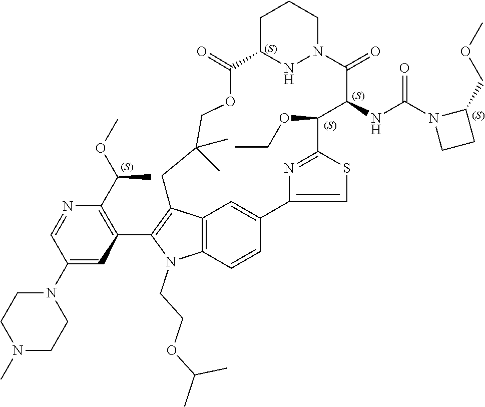 |
| DA460 | 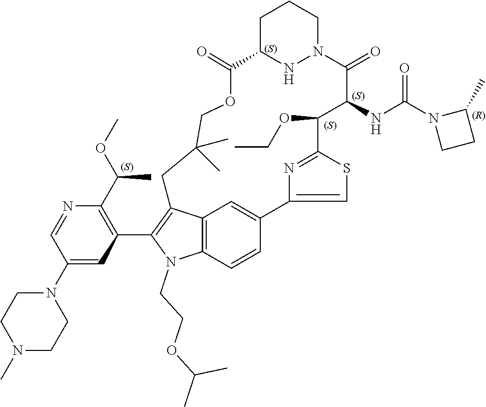 |
| DA461 | 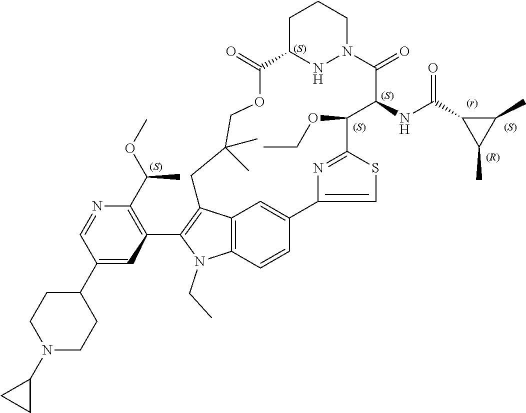 |
| DA462 | 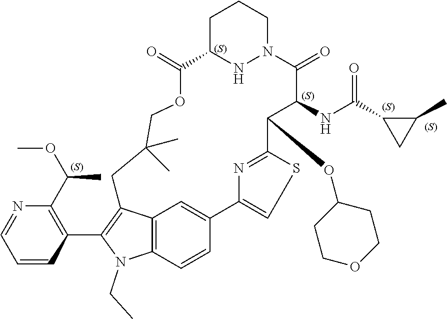 |
| DA463 | 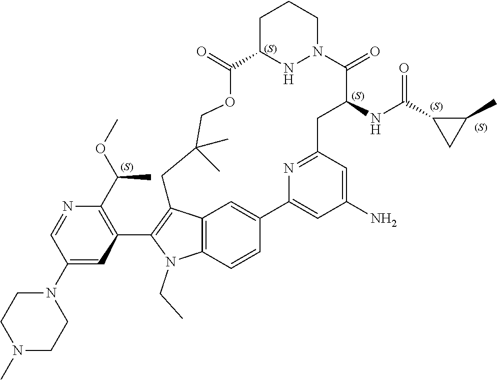 |
| DA464 | 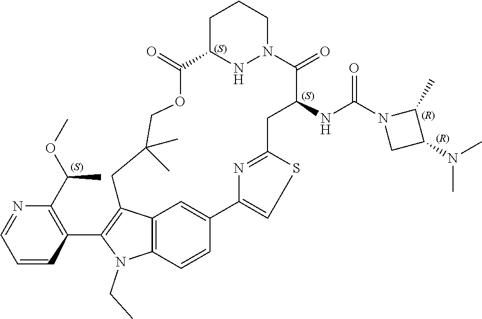 |
| DA465 | 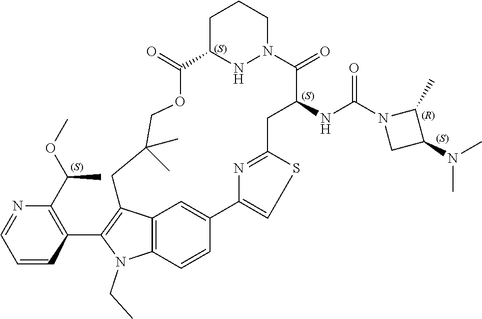 |
| DA466 | 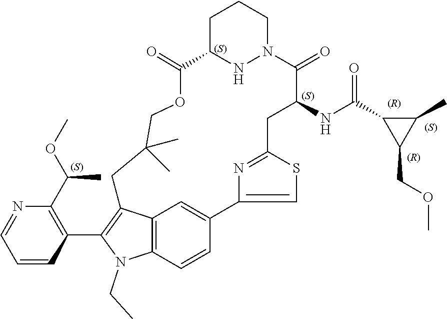 |
| DA467 | 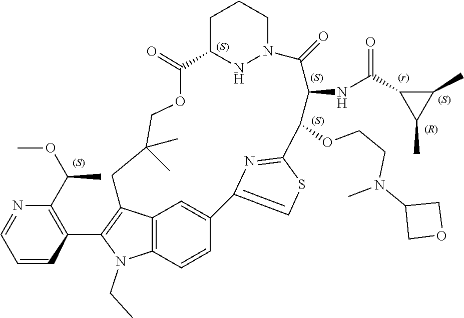 |
| DA468 | 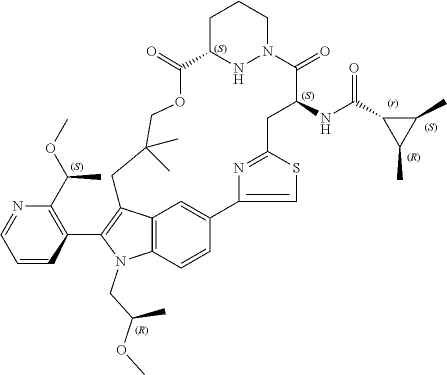 |
| DA469 | 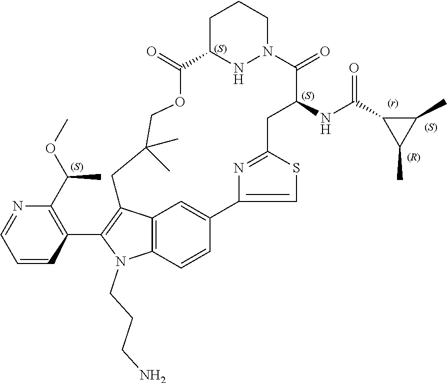 |
| DA470 | 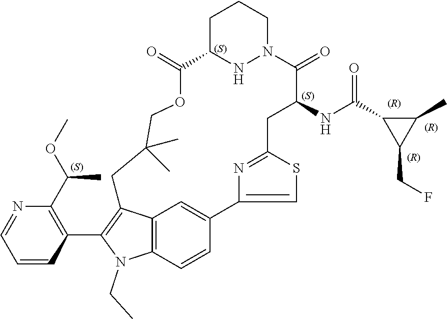 |
| DA471 | 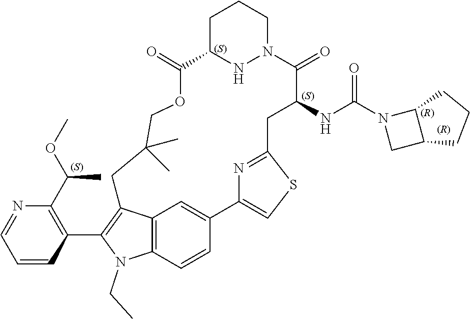 |
| DA472 | 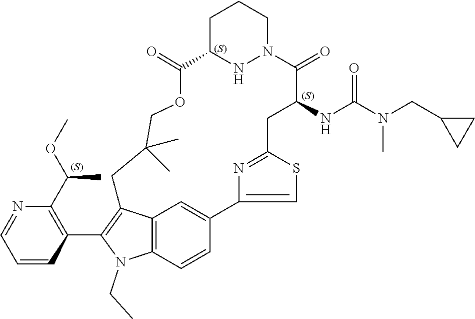 |
| DA473 | 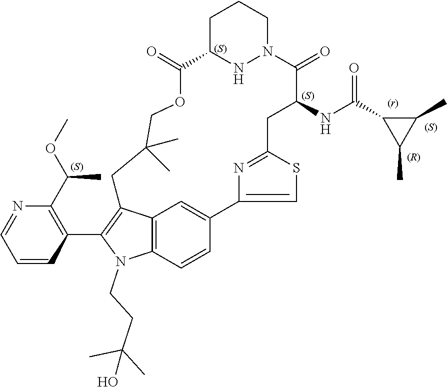 |
| DA474 | 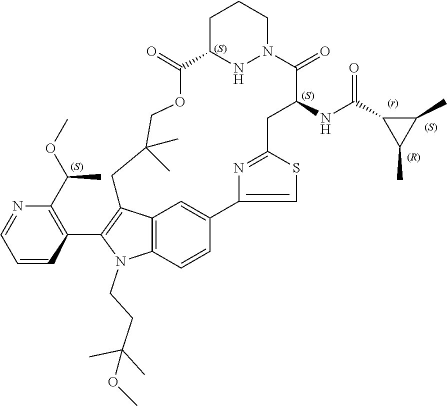 |
| DA475 | 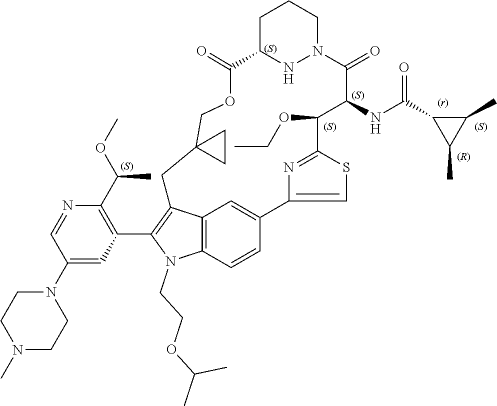 |
| DA476 | 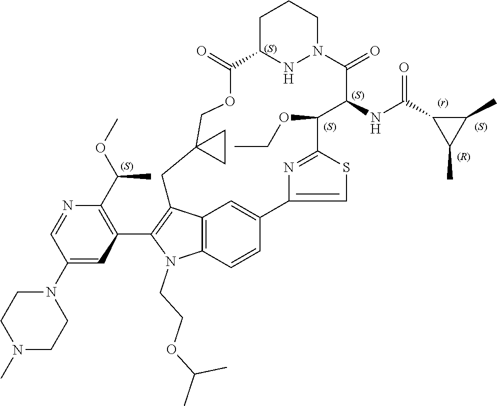 |
| DA477 | 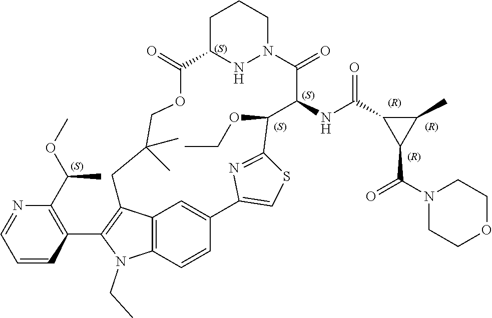 |
| DA478 | 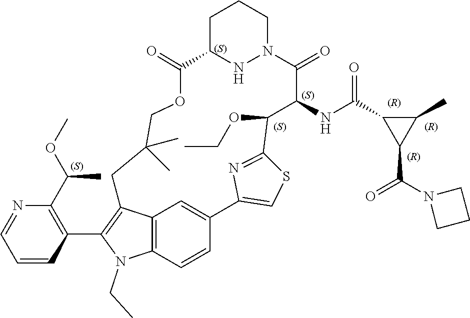 |
| DA479 | 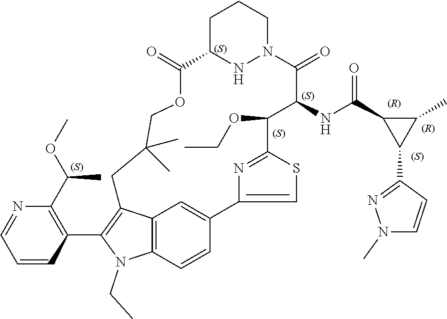 |
| DA480 | 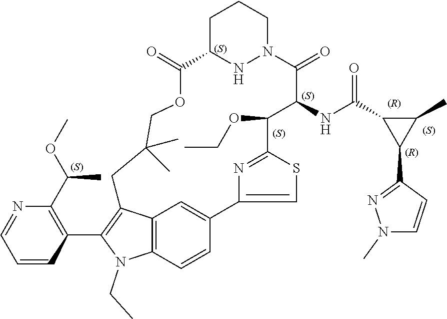 |
| DA481 | 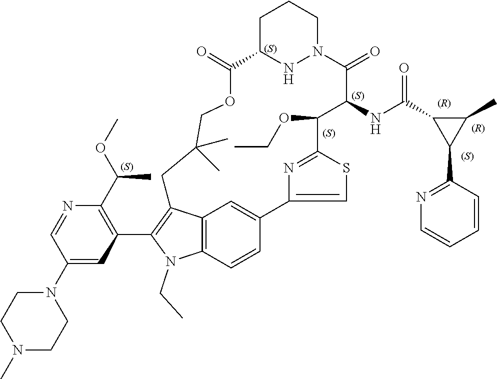 |
| DA482 | 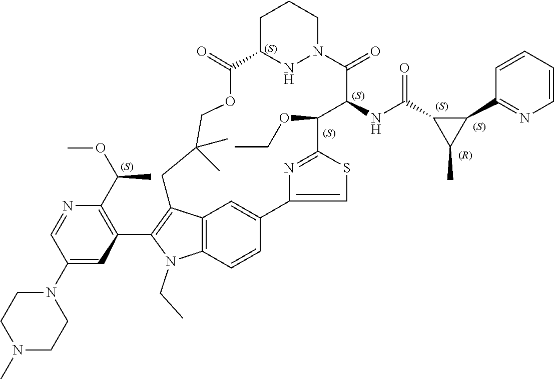 |
| DA483 | 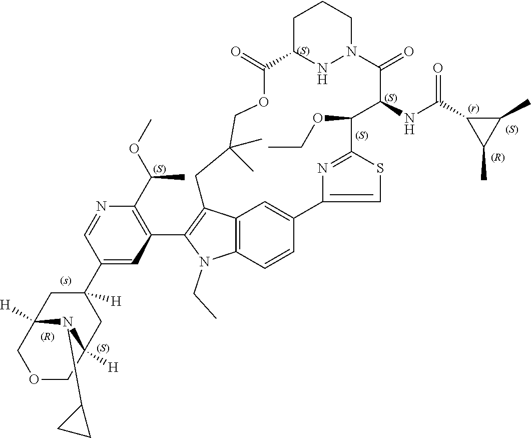 |
| DA484 | 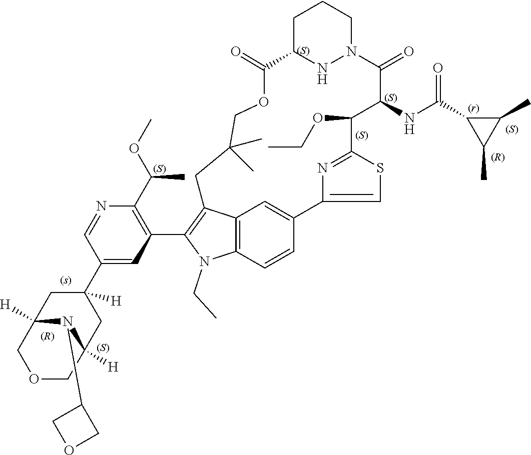 |
| DA485 | 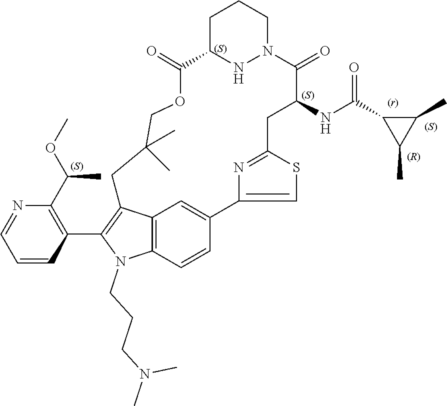 |
| DA486 | 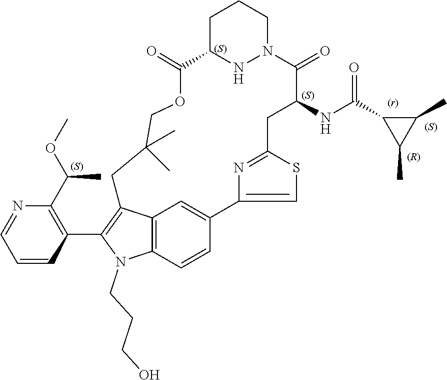 |
| DA487 | 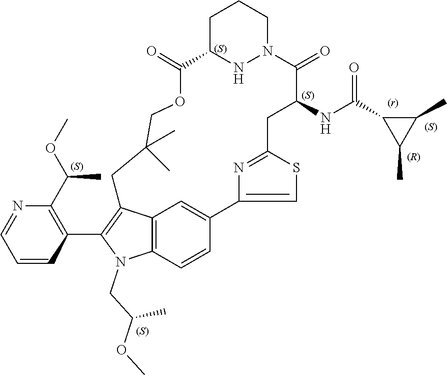 |
| DA488 | 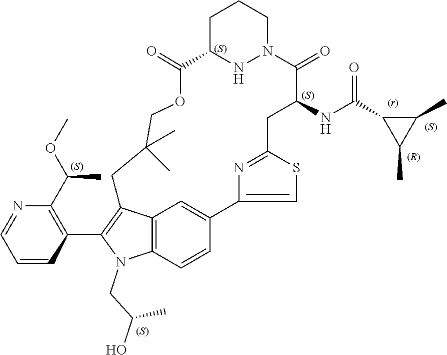 |
| DA489 | 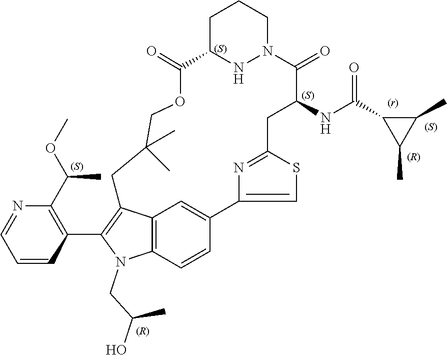 |
| DA490 | 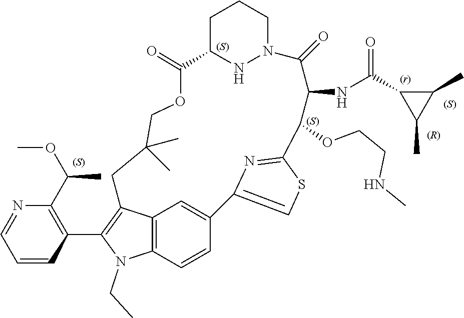 |
| DA491 | 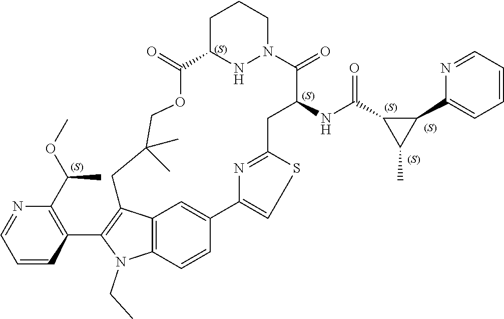 |
| DA492 | 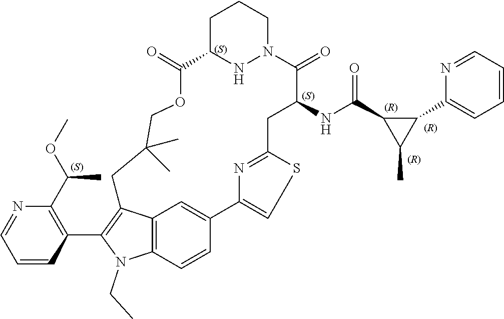 |
| DA493 | 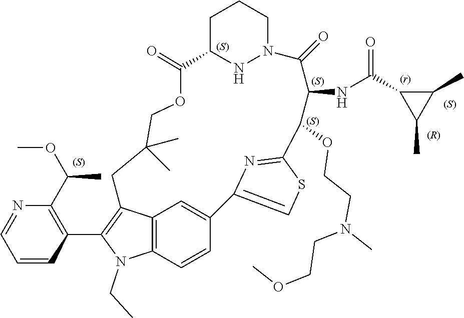 |
| DA494 | 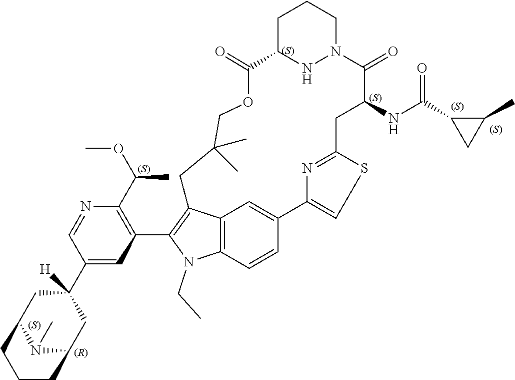 |
| DA495 | 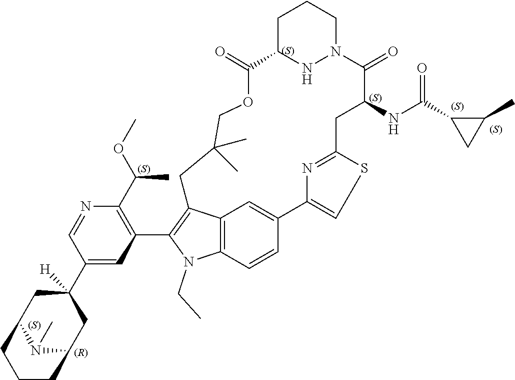 |
| DA496 | 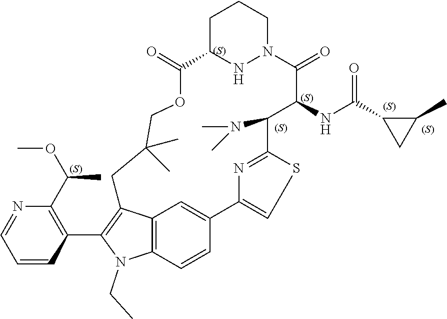 |
| DA497 | 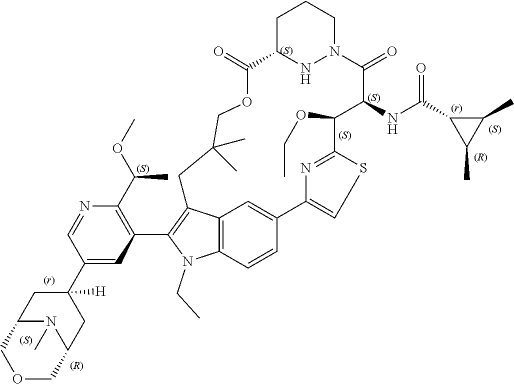 |
| DA498 | 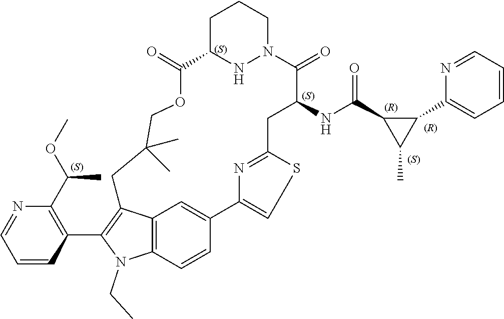 |
| DA499 | 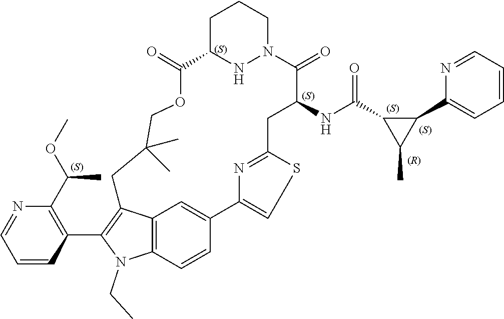 |
| DA500 | 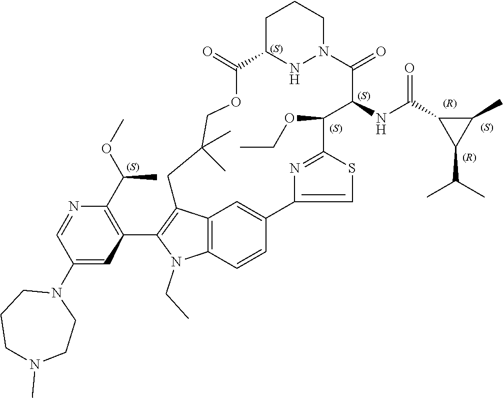 |
| DA501 | 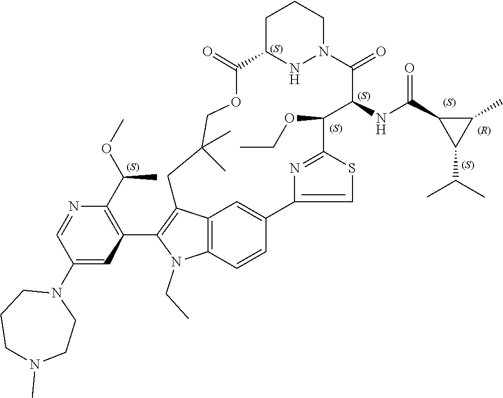 |
| DA502 | 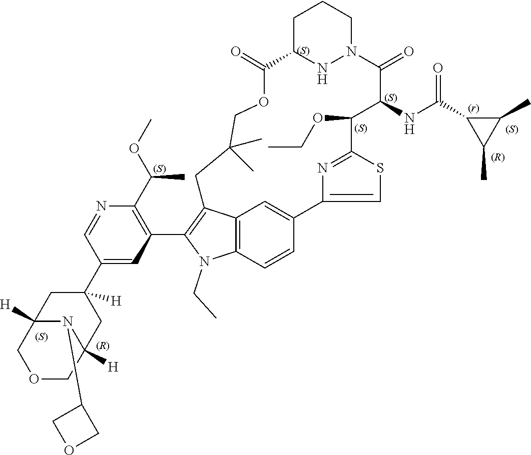 |
| DA503 | 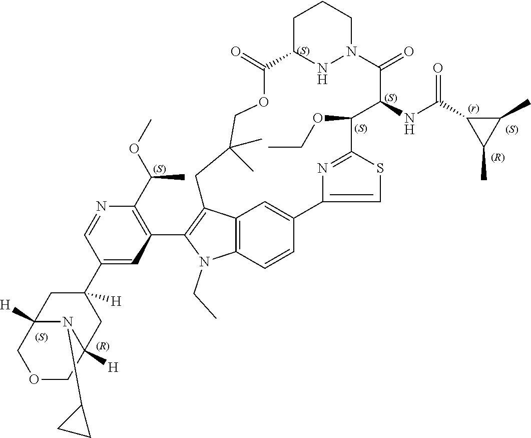 |
| DA504 | 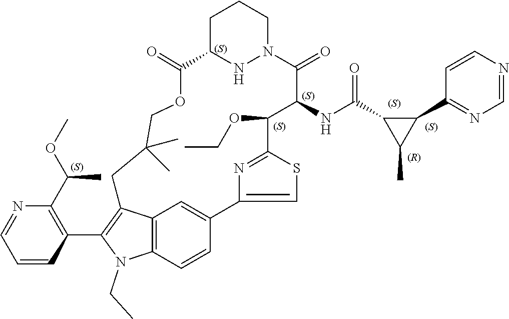 |
| DA505 | 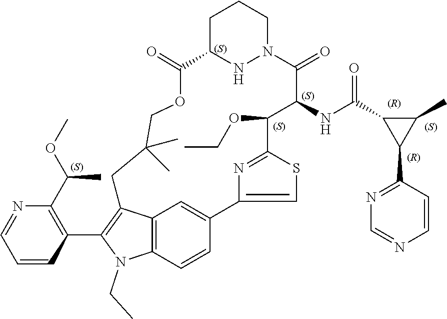 |
| DA506 | 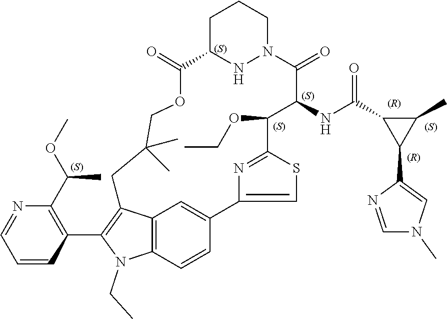 |
| DA507 | 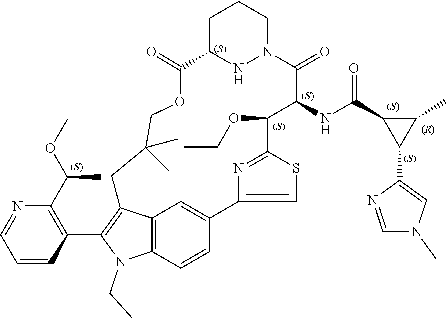 |
| DA508 | 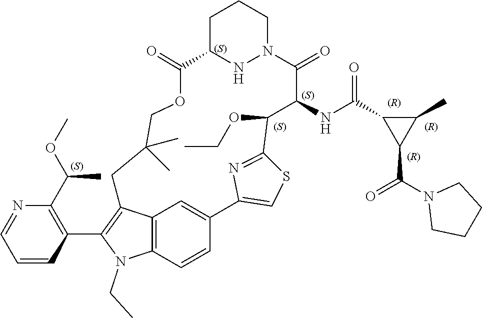 |
| DA509 | 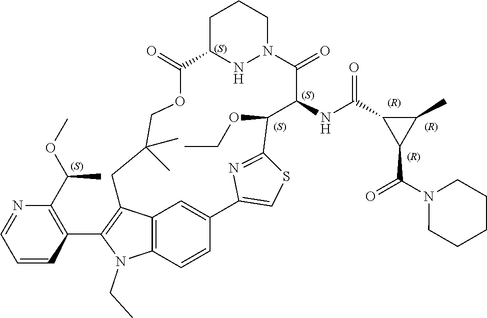 |
| DA510 | 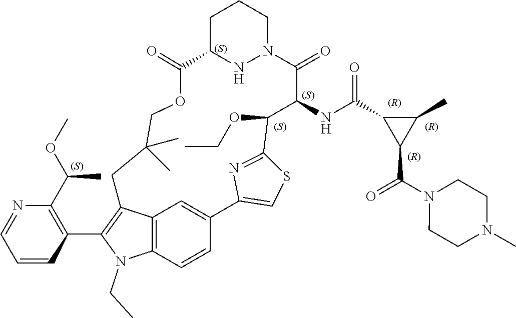 |
| DA511 | 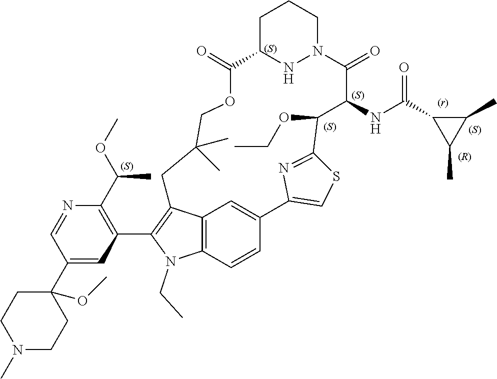 |
| DA512 | 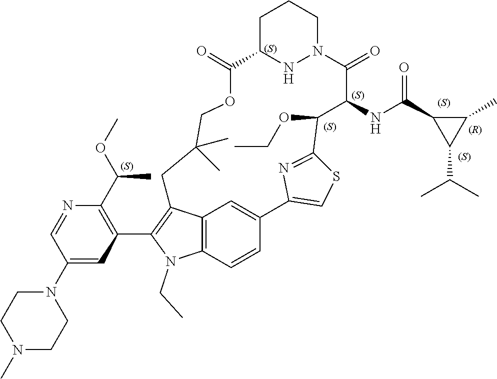 |
| DA513 | 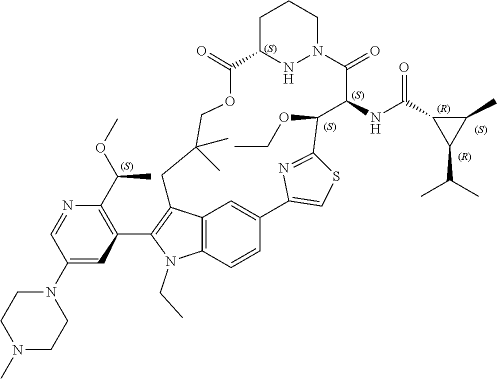 |
| DA514 | 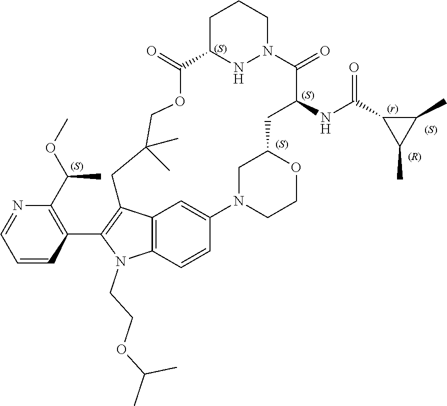 |
| DA515 | 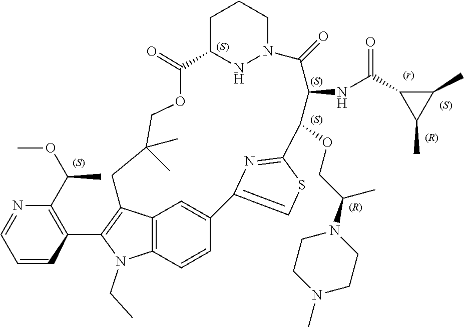 |
| DA516 | 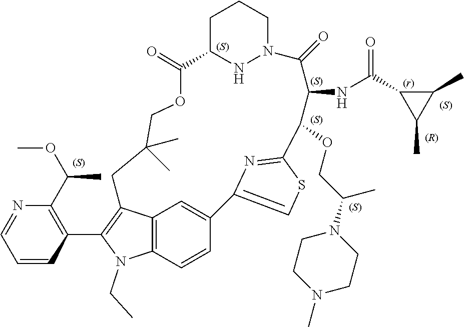 |
| DA517 | 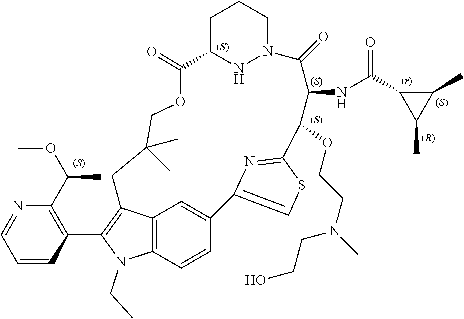 |
| DA518 | 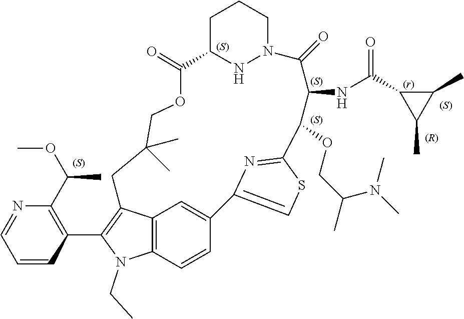 |
| DA519 | 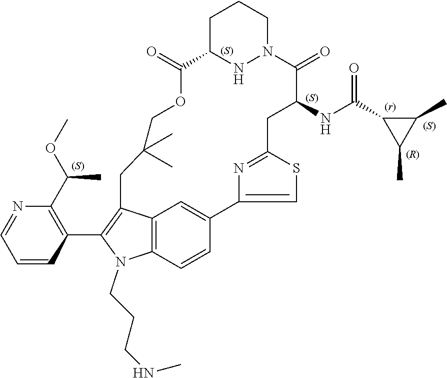 |
| DA520 | 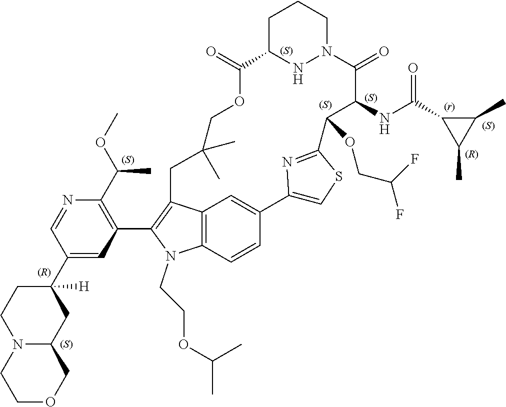 |
| DA521 | 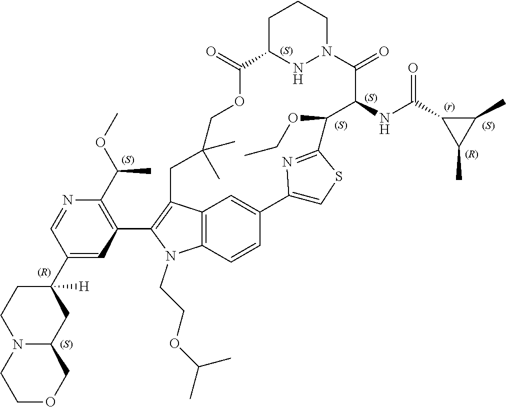 |
| DA522 | 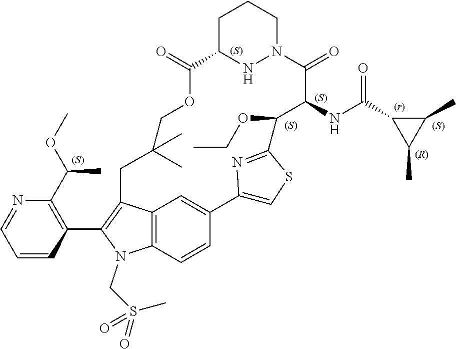 |
| DA523 | 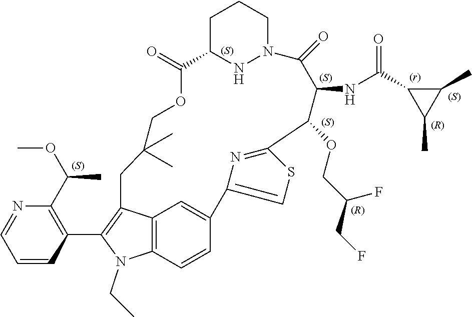 |
| DA524 | 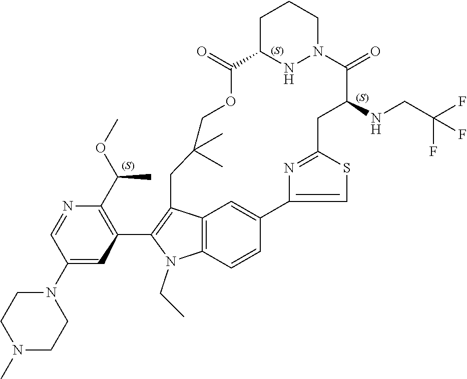 |
| DA525 | 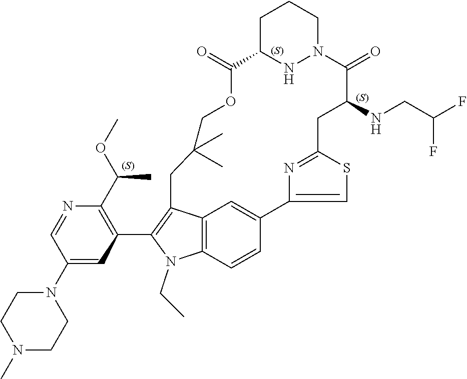 |
| DA526 | 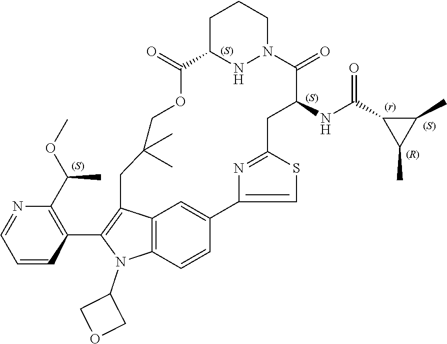 |
| DA527 | 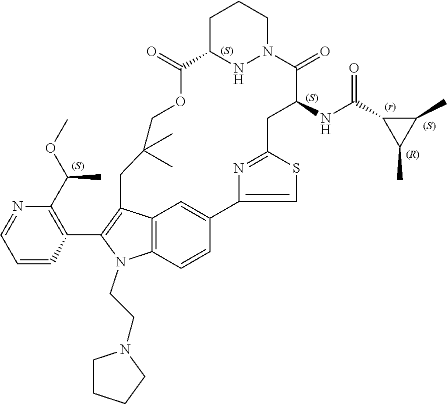 |
| DA528 | 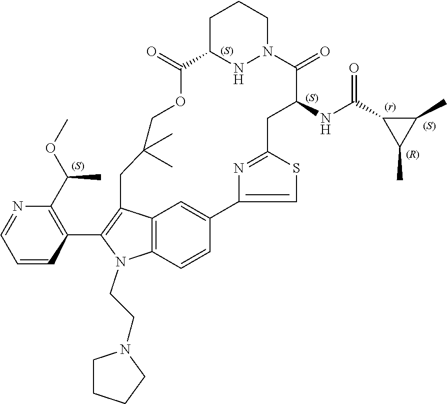 |
| DA529 | 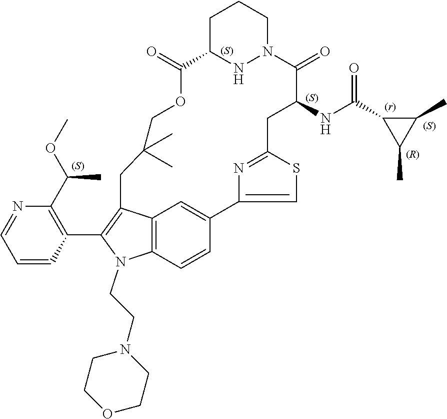 |
| DA530 | 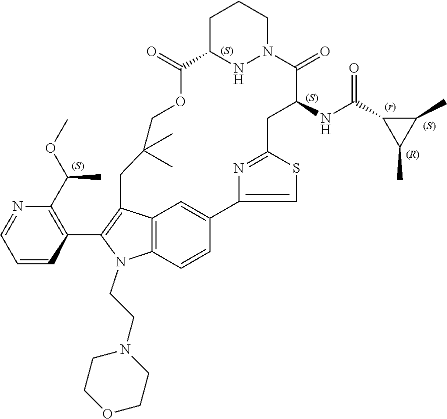 |
| DA531 | 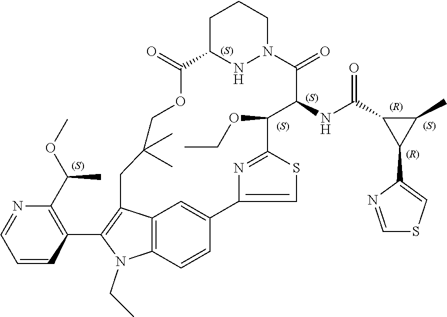 |
| DA532 | 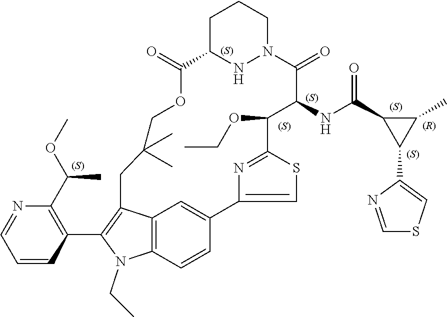 |
| DA533 | 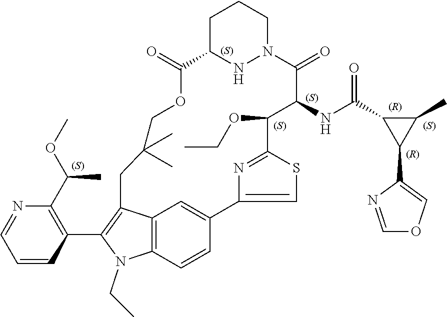 |
| DA534 | 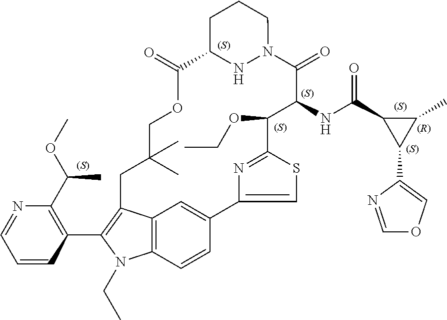 |
| DA535 | 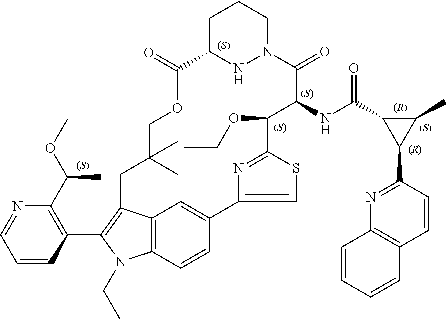 |
| DA536 | 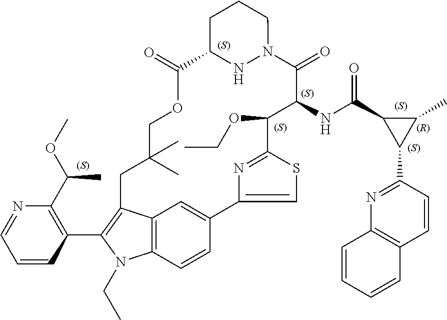 |
| DA537 | 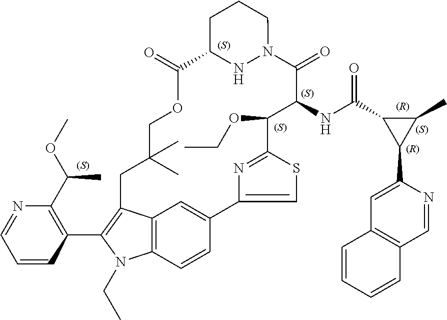 |
| DA538 | 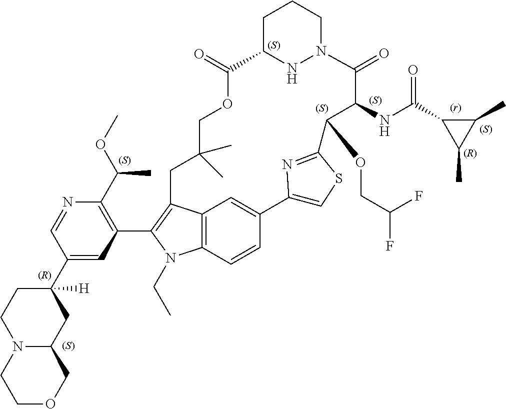 |
| DA539 | 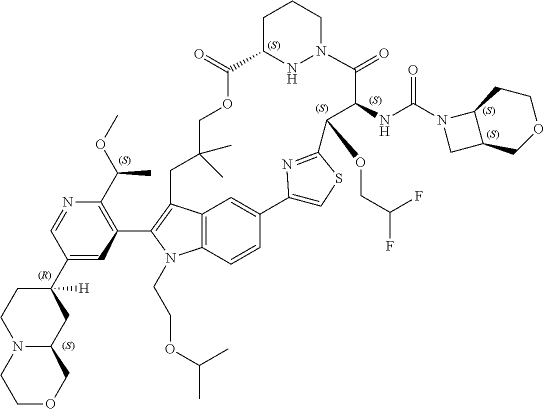 |
| DA540 | 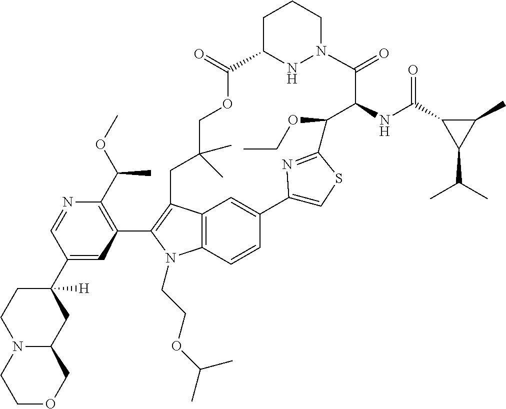 |
| DA541 | 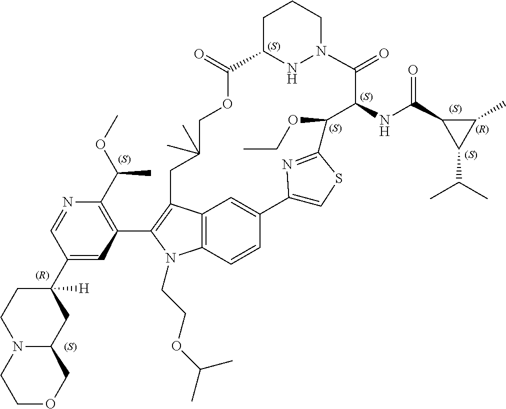 |
| DA542 | 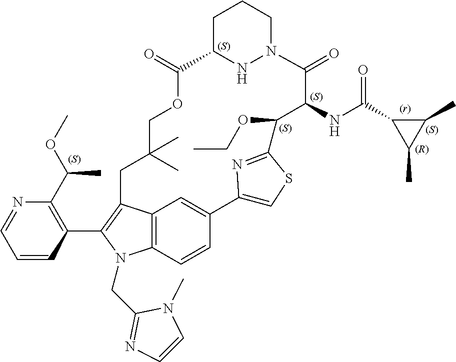 |
| DA543 | 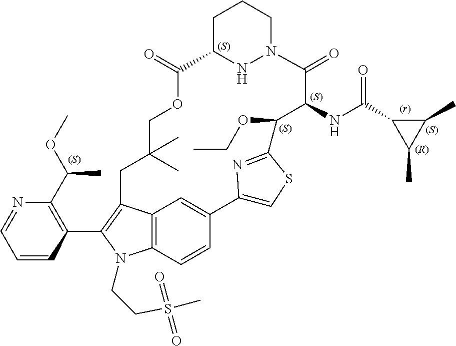 |
| DA544 | 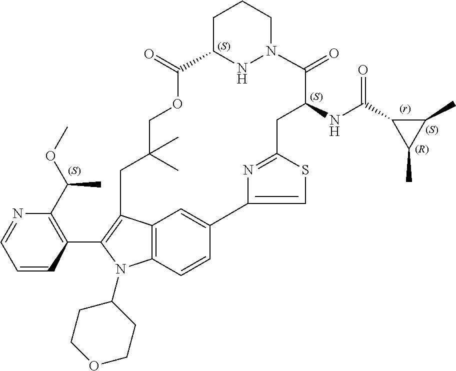 |
| DA545 | 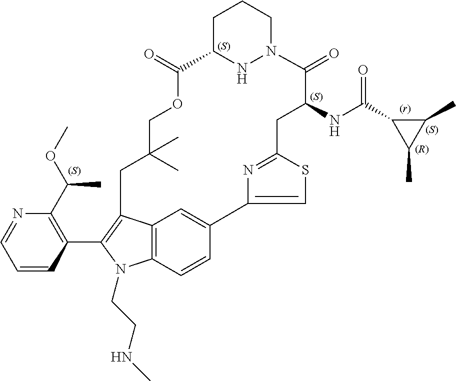 |
| DA546 | 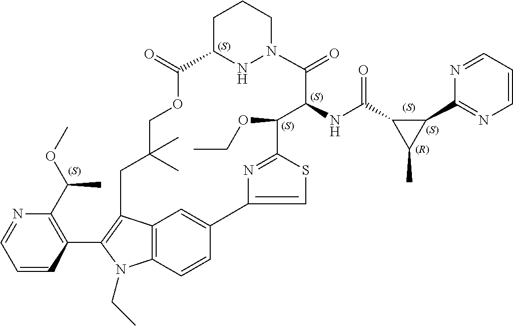 |
| DA547 | 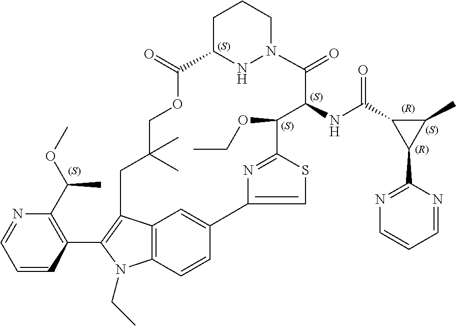 |
| DA548 | 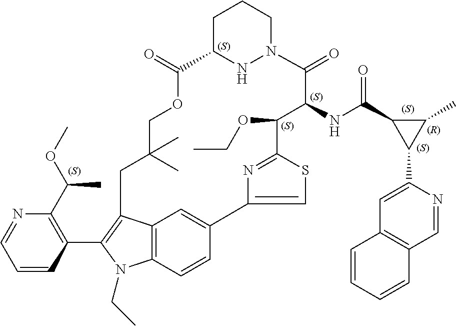 |
| DA549 | 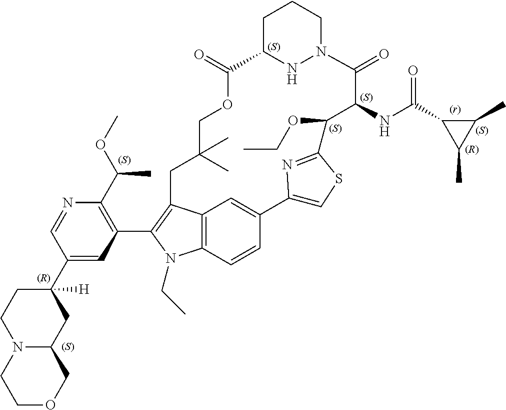 |
| DA550 | 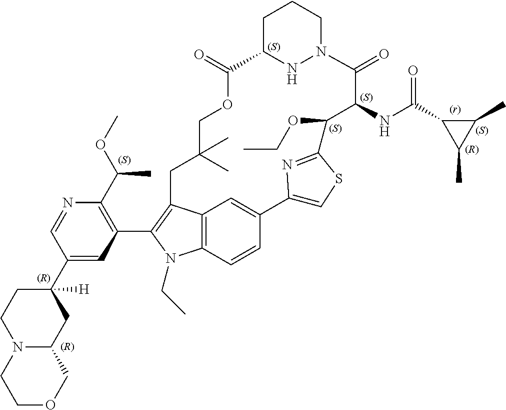 |
| DA551 | 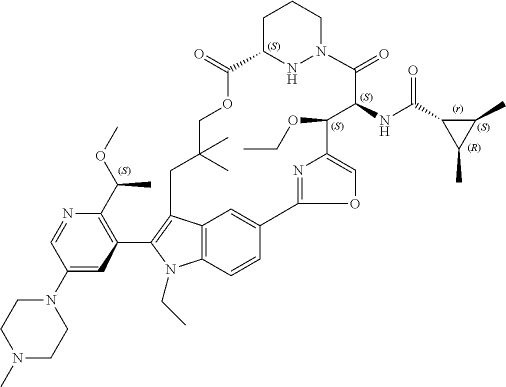 |
| DA552 | 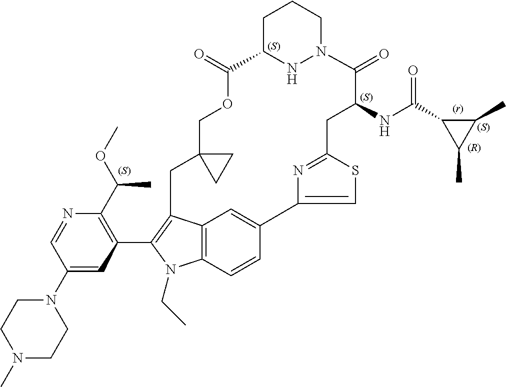 |
| DA553 | 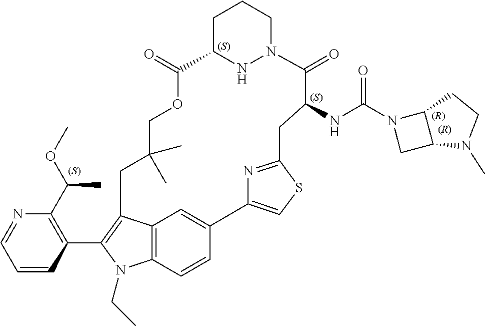 |
| DA554 | 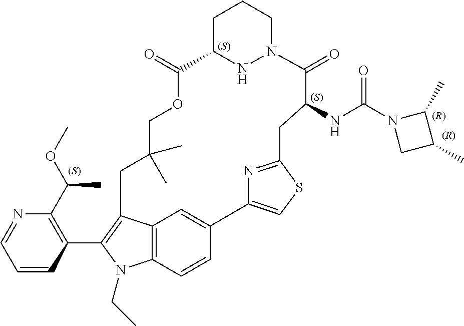 |
| DA555 | 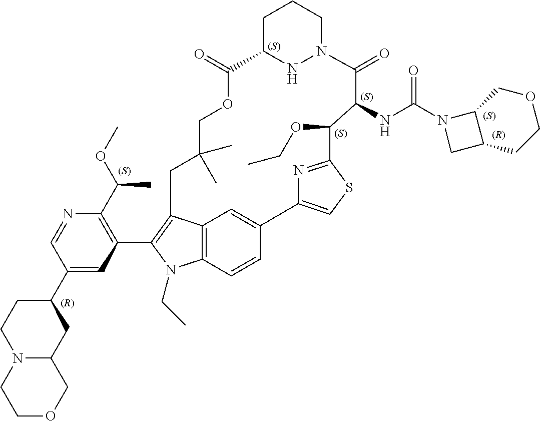 |
| DA556 | 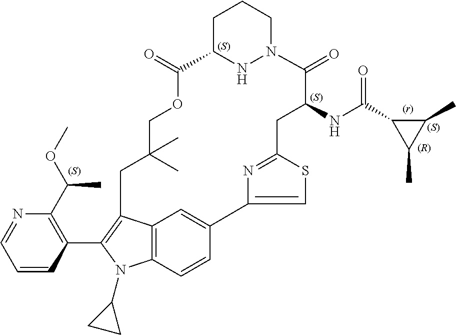 |
| DA557 | 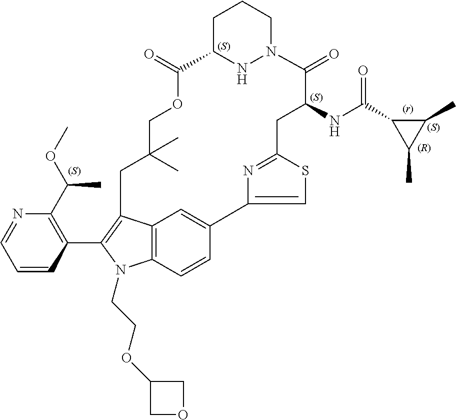 |
| DA558 | 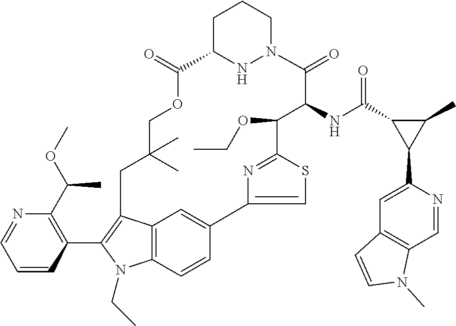 |
| DA559 | 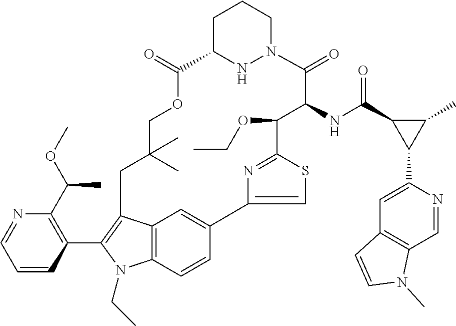 |
| DA560 | 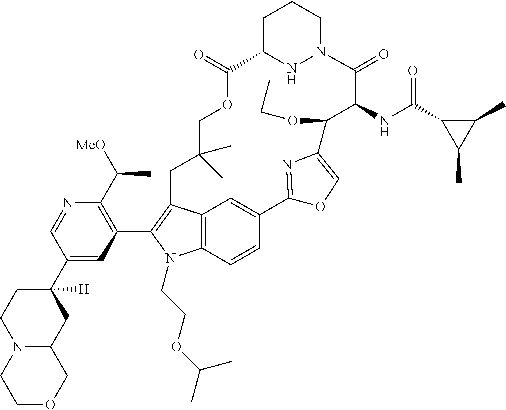 |
| DA561 | 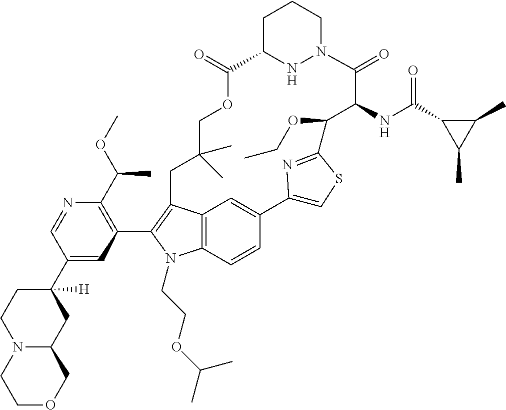 |
| DA562 | 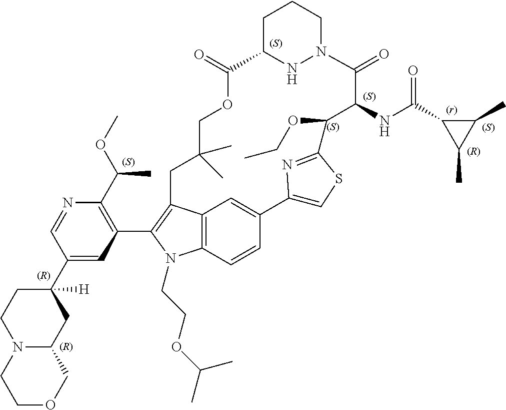 |
| DA563 | 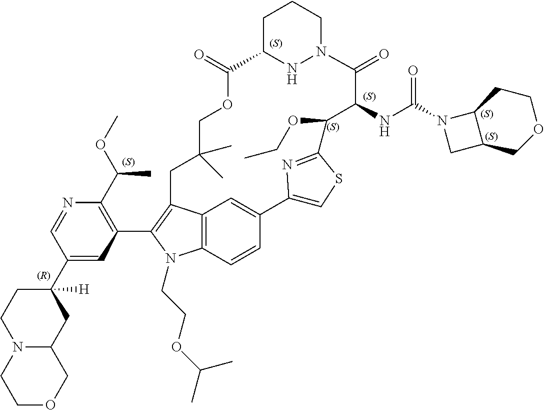 |
| DA564 | 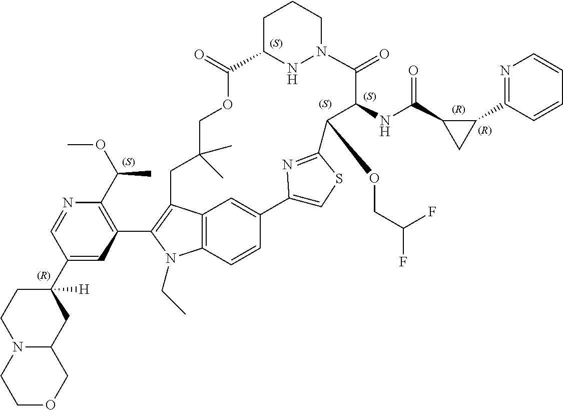 |
| DA565 | 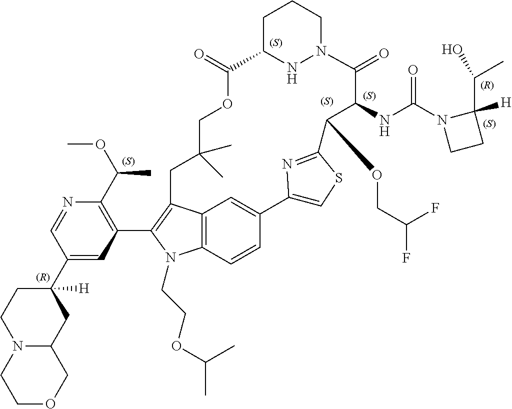 |
| DA566 | 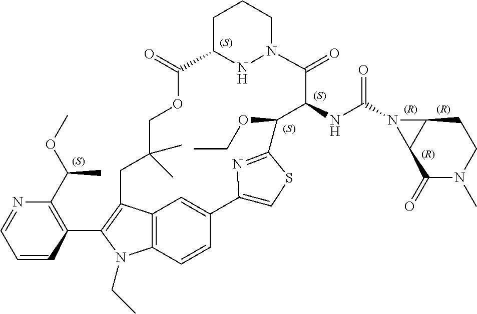 |
| DA567 | 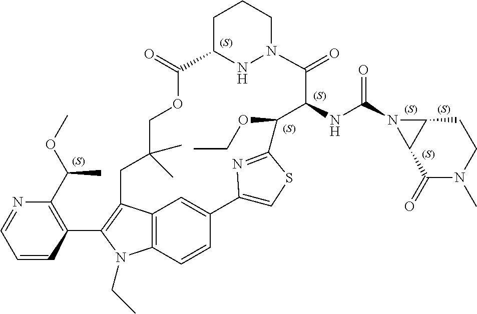 |
| DA568 | 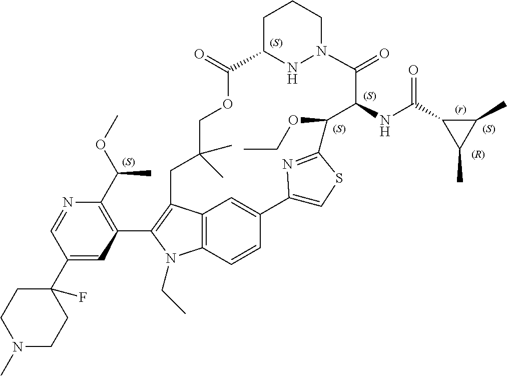 |
| DA569 | 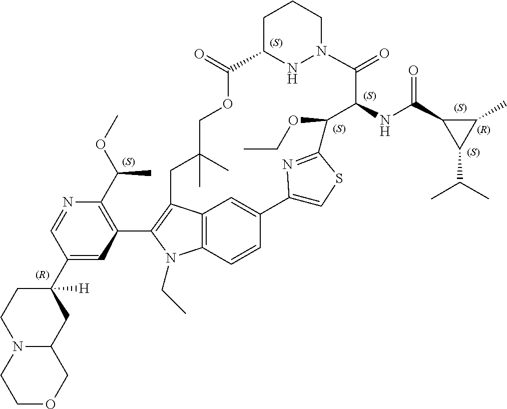 |
| DA570 | 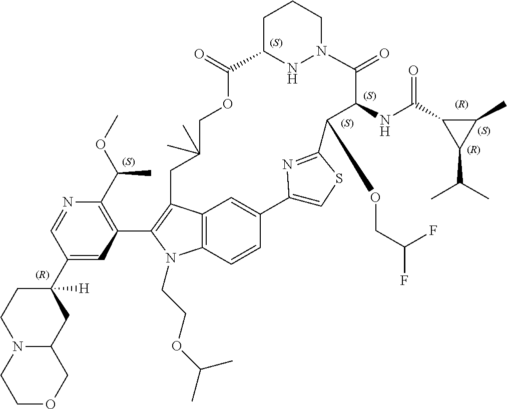 |
| DA571 | 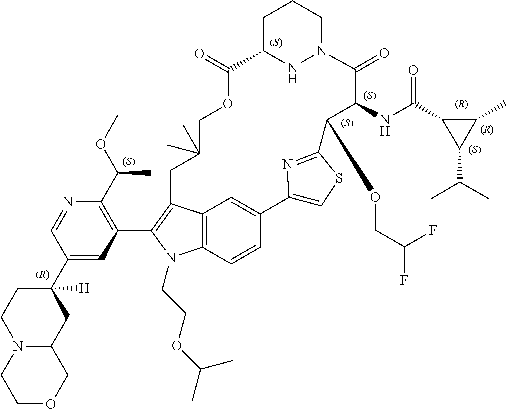 |
| DA572 | 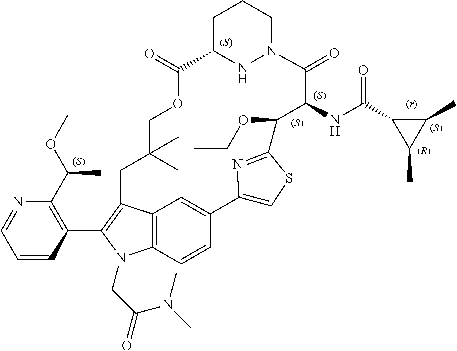 |
| DA573 | 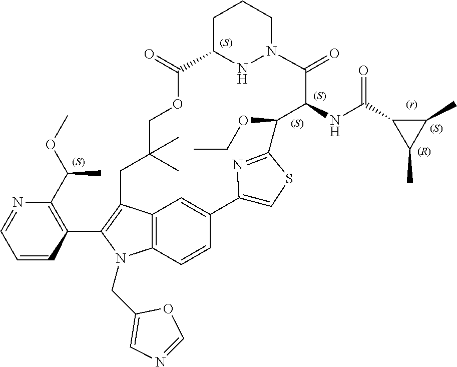 |
| DA574 | 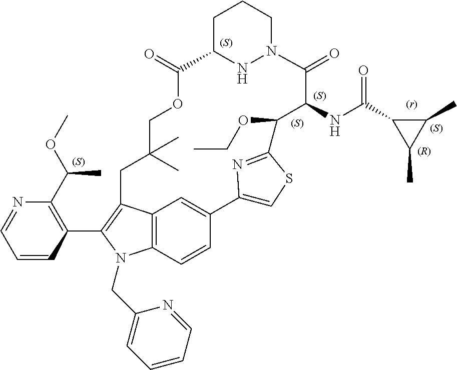 |
| DA575 | 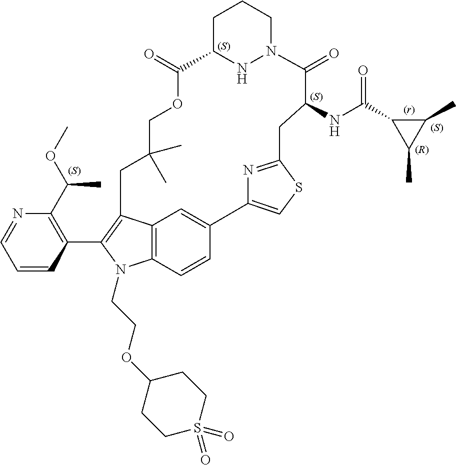 |
| DA576 | 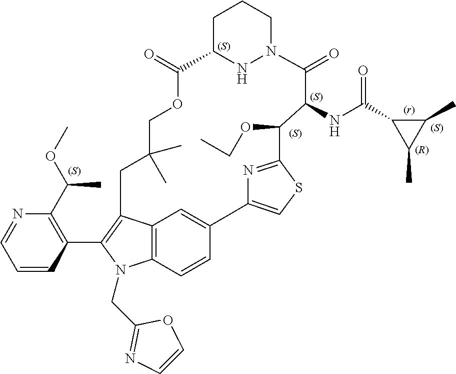 |
| DA577 | 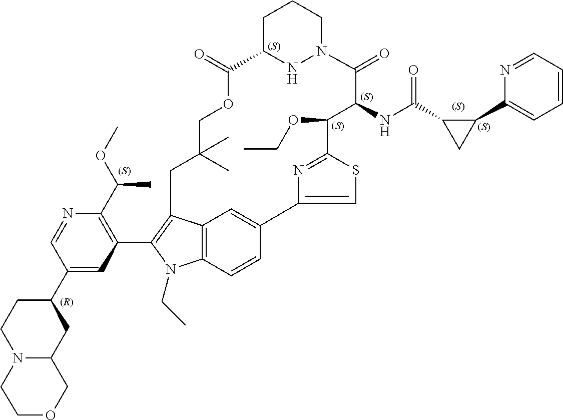 |
| DA578 | 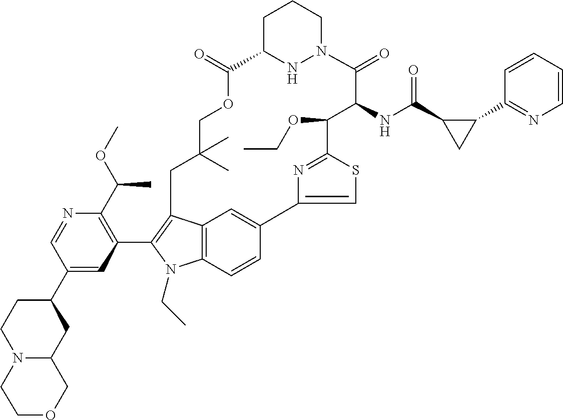 |
| DA579 | 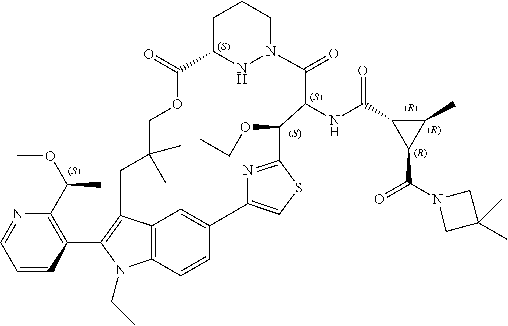 |
| DA580 | 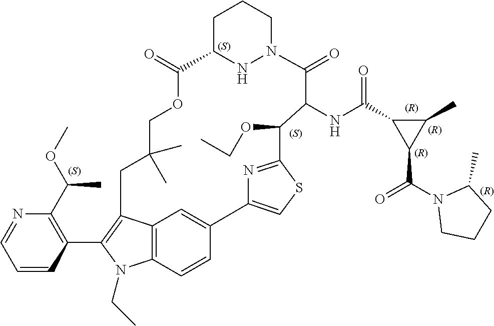 |
| DA581 | 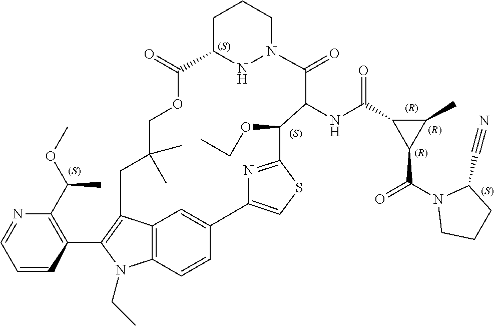 |
| DA582 | 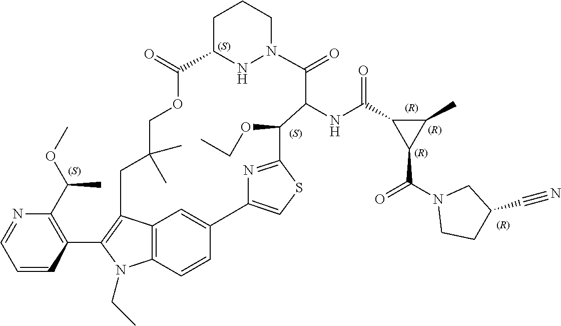 |
| DA583 | 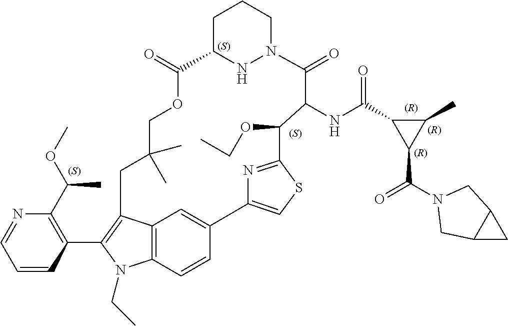 |
| DA584 | 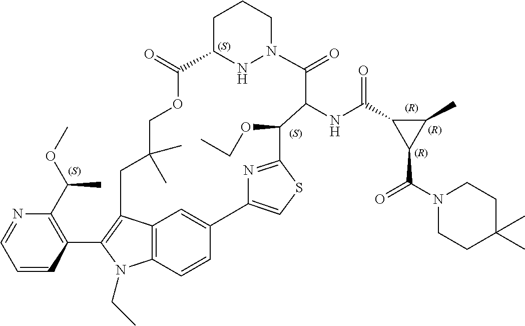 |
| DA585 | 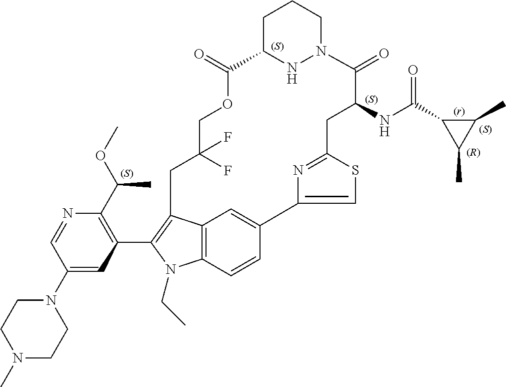 |
| DA586 | 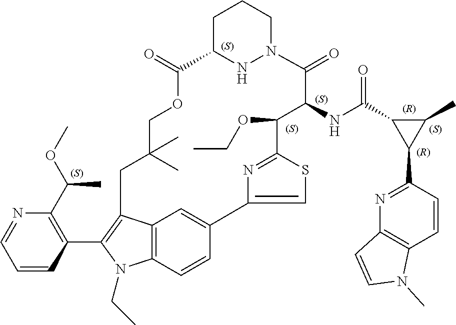 |
| DA587 | 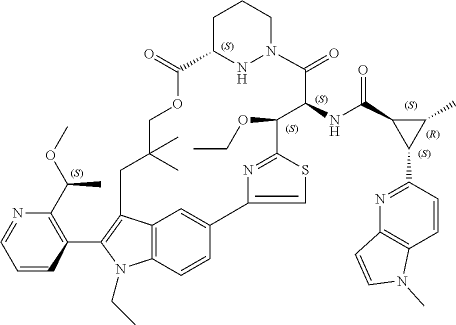 |
| DA588 | 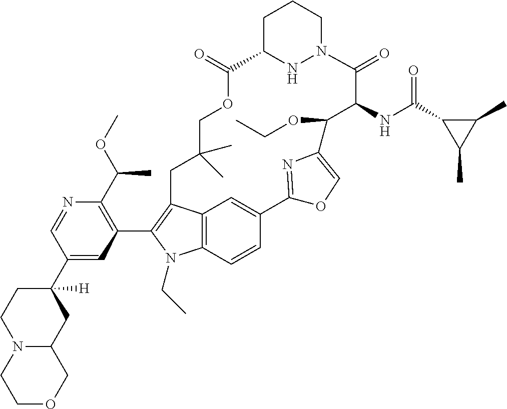 |
| DA589 | 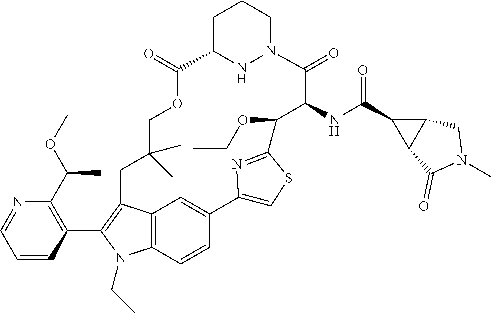 |
| DA590 | 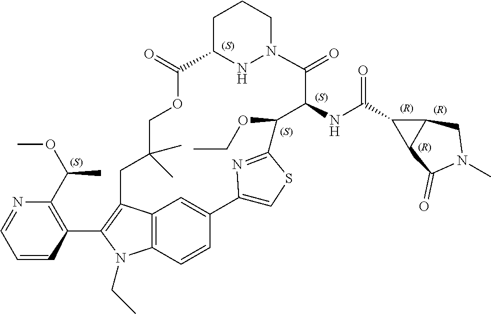 |
| DA591 | 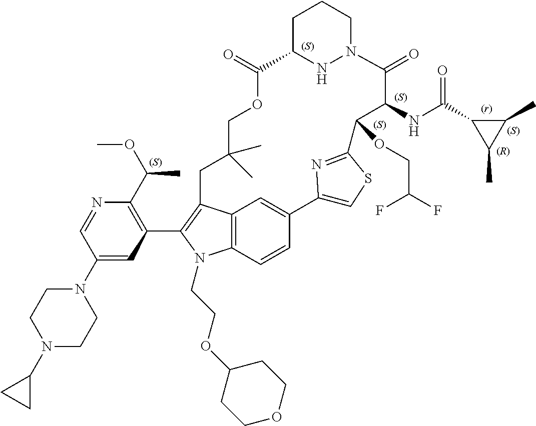 |
| DA592 | 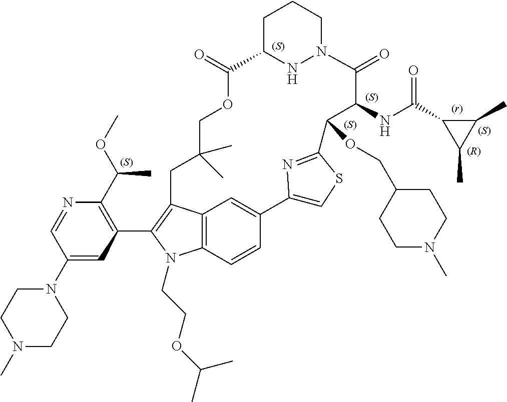 |
| DA593 | 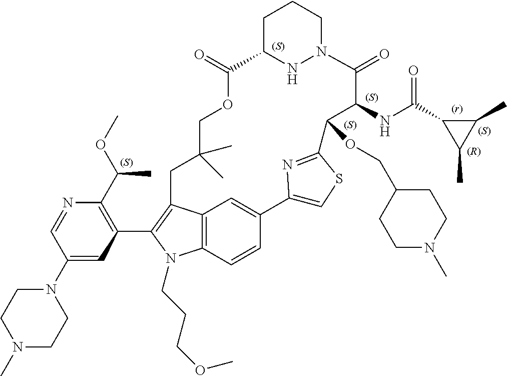 |
| DA594 | 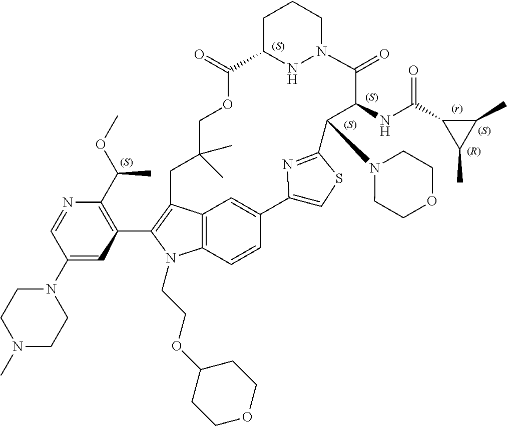 |
| DA595 | 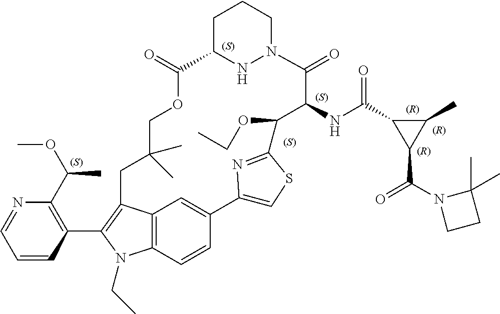 |
| DA596 | 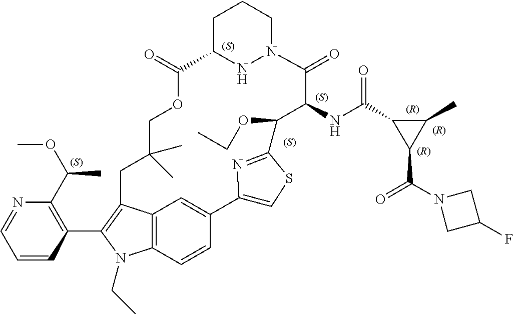 |
| DA597 | 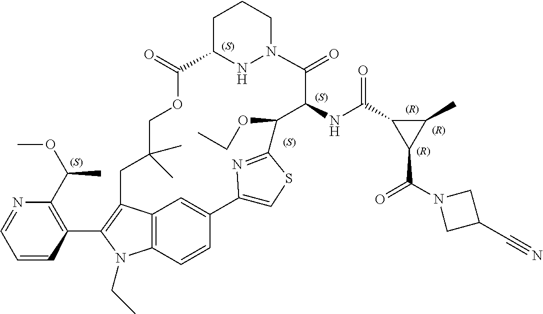 |
| DA598 | 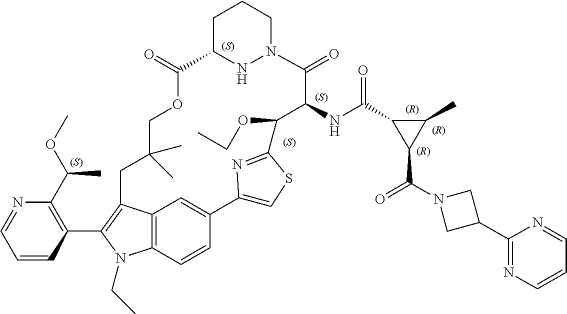 |
| DA599 | 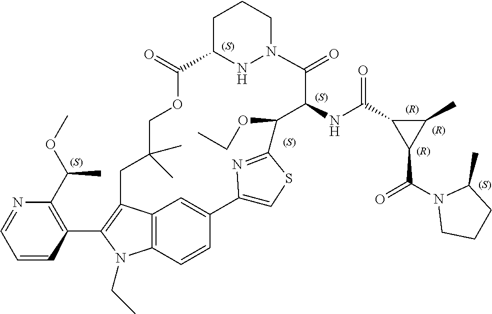 |
| DA600 | 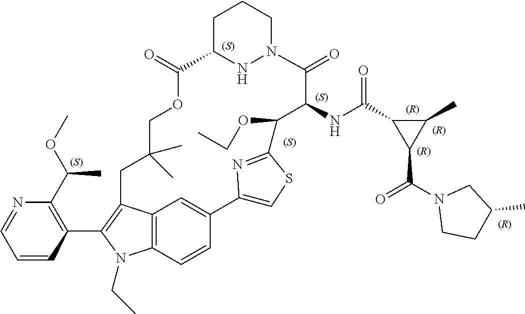 |
| DA601 | 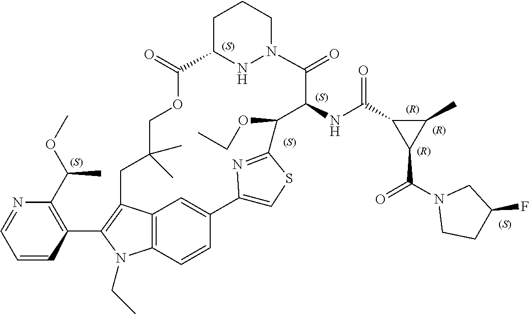 |
| DA602 | 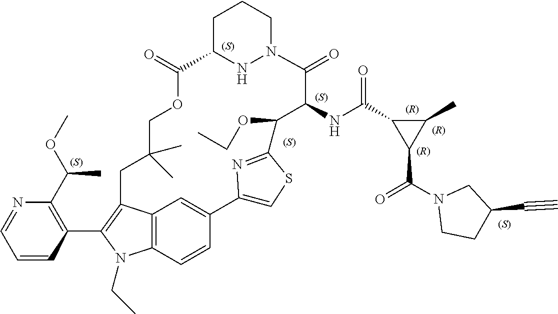 |
| DA603 | 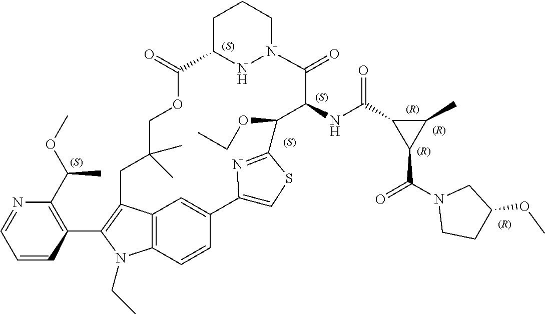 |
| DA604 | 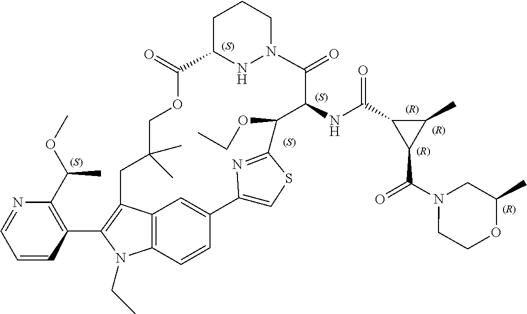 |
| DA605 | 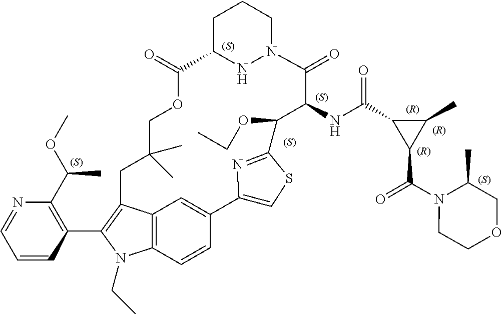 |
| DA606 | 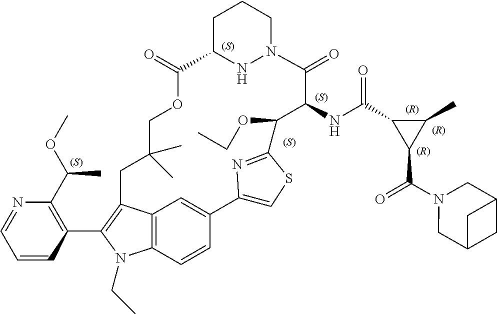 |
| DA607 | 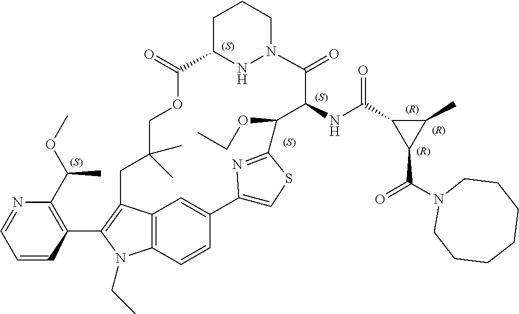 |
| DA608 | 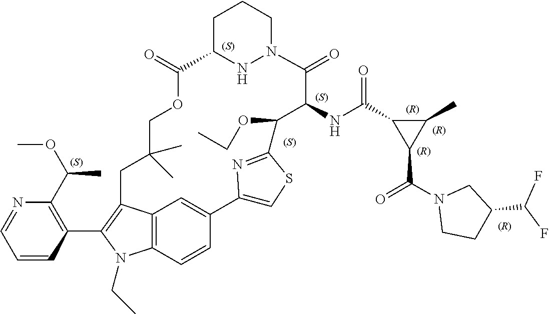 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, the RAS(ON) inhibitor is a compound selected from Table D2, or a pharmaceutically acceptable salt or stereoisomer thereof. In some embodiments, the RAS(ON) inhibitor is a compound selected from Table D2, or a pharmaceutically acceptable salt or atropisomer thereof.

In some embodiments, the RAS(ON) inhibitor is not a compound selected from Table D2. In some embodiments, the RAS(ON) inhibitor is not a compound selected from Table D2, or a pharmaceutically acceptable salt or stereoisomer thereof. In some embodiments, the RAS(ON) inhibitor is not a compound selected from Table D2, or a pharmaceutically acceptable salt or atropisomer thereof.

TABLE D2
Certain Compounds

| Ex. # | Structure |
|---|---|
| DB1 | 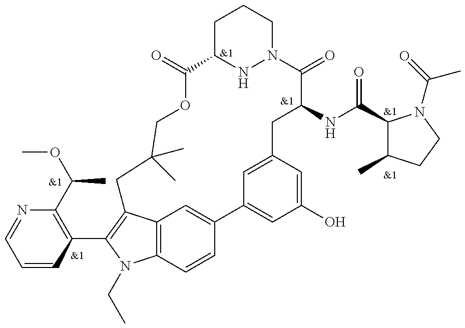 |
| DB2 | 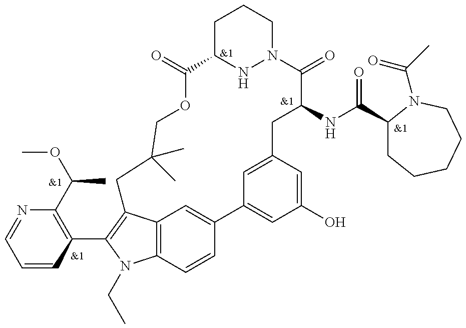 |
| DB3 | 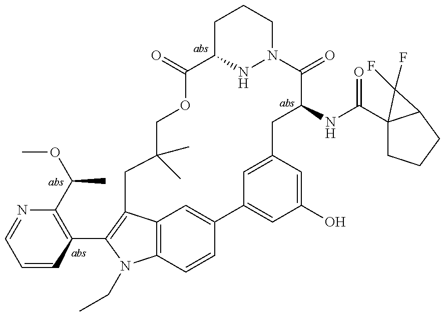 |
| DB4 | 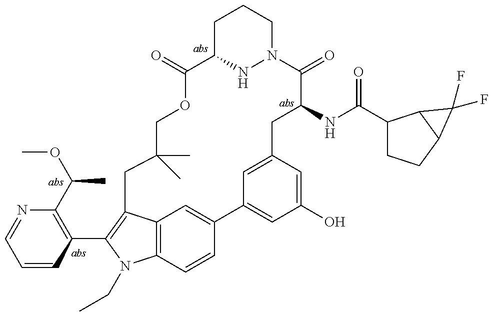 |
| DB5 | 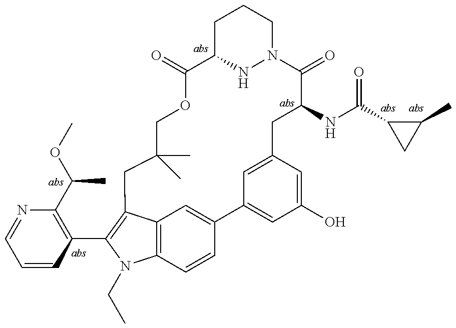 |
| DB6 | 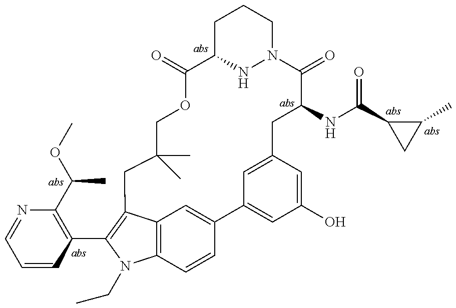 |
| DB7 | 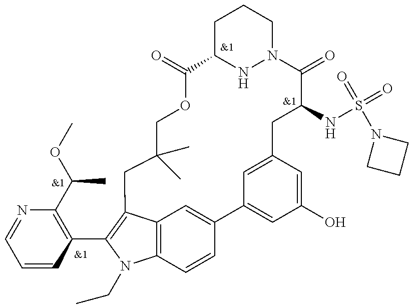 |
| DB8 | 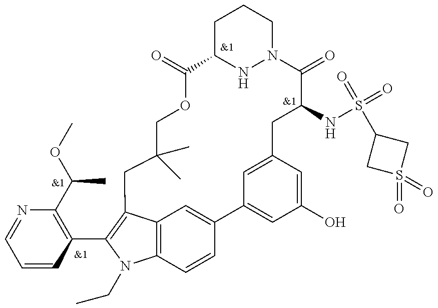 |
| DB9 | 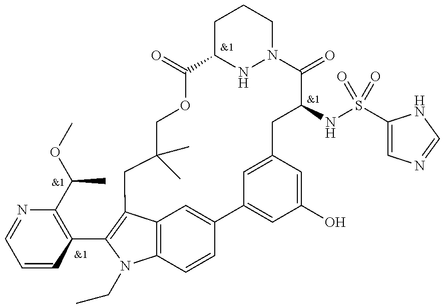 |
| DB10 | 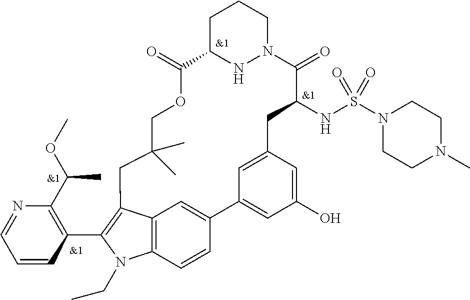 |
| DB11 | 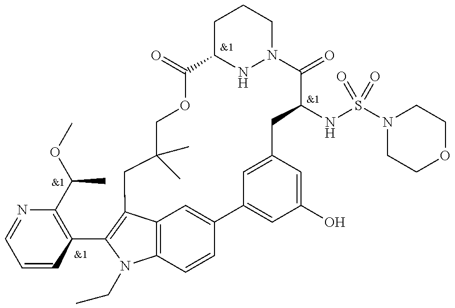 |
| DB12 | 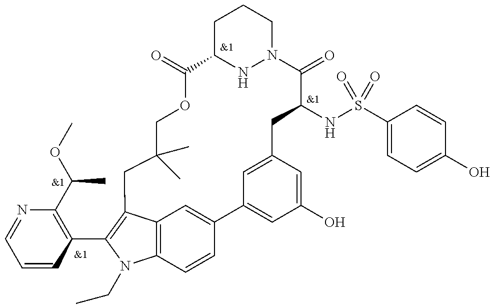 |
| DB13 | 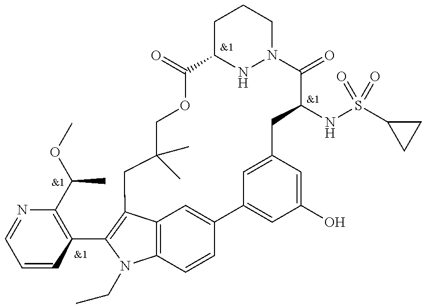 |
| DB14 | 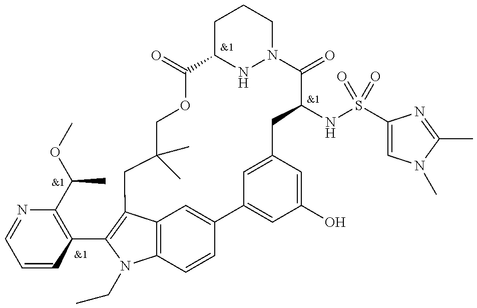 |
| DB15 | 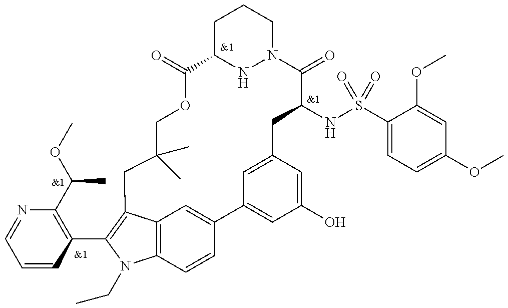 |
| DB16 | 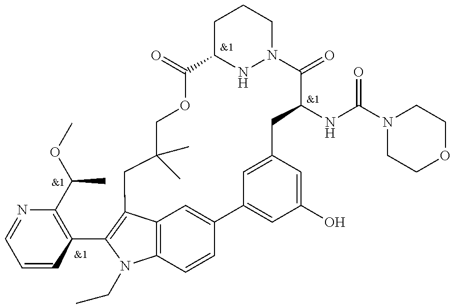 |
| DB17 | 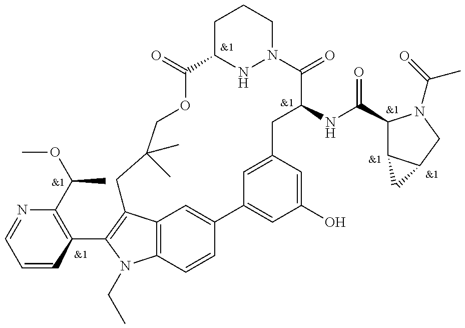 |
| DB18 | 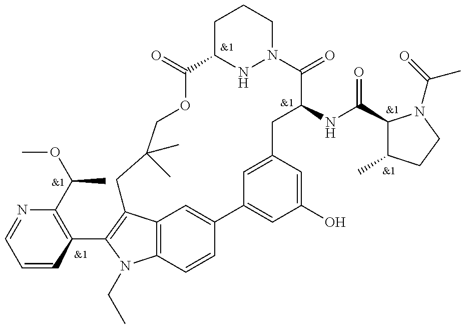 |
| DB19 | 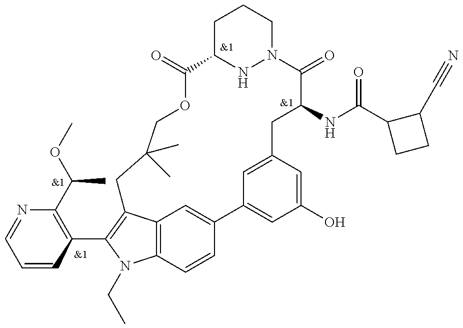 |
| DB20 | 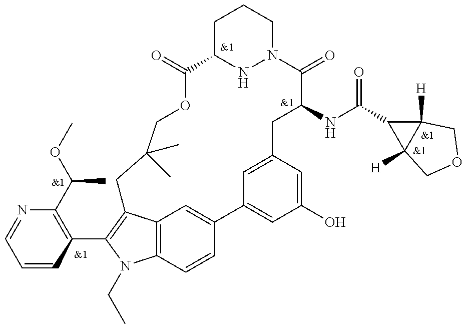 |
| DB21 | 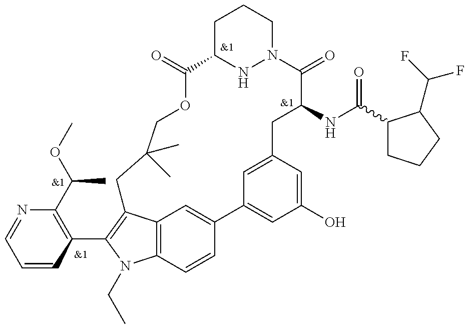 |
| DB22 | 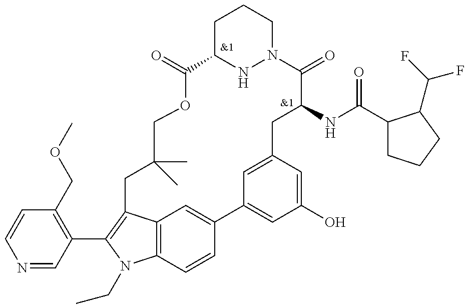 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| DB23 | 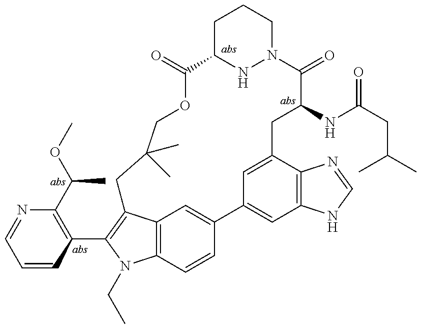 |
| DB24 | 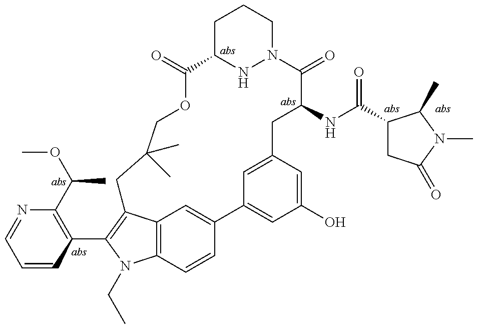 |
| DB25 | 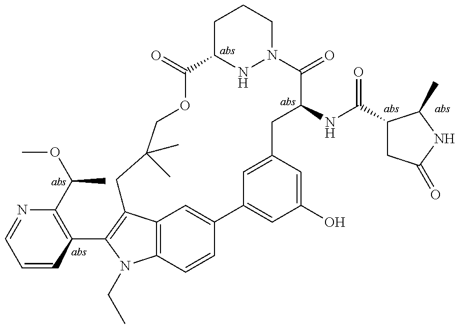 |

In some embodiments, a compound of the present invention is a compound selected from Table D3 (e.g., DC1-DC20 or DC1-DC21), or a pharmaceutically acceptable salt or stereoisomer thereof In some embodiments, a compound of the present invention is a compound selected from Table D3 (e.g., DC1-DC20 or DC1-DC21), or a pharmaceutically acceptable salt or atropisomer thereof.

In some embodiments, a compound of the present invention is not a compound selected from Table D3 (e.g., DC1-DC20 or DC1-DC21). In some embodiments, a compound of the present invention is not a compound selected from Table D3 (e.g., DC1-DC20 or DC1-DC21), or a pharmaceutically acceptable salt or stereoisomer thereof. In some embodiments, a compound of the present invention is not a compound selected from Table D3 (e.g., DC1-DC20 or DC1-DC21), or a pharmaceutically acceptable salt or atropisomer thereof

TABLE D3
Certain Compounds

| Ex. # | Structure |
|---|---|
| DC1 | 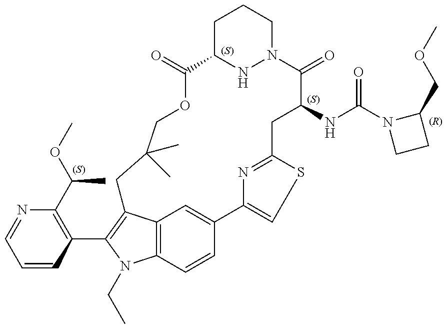 |
| DC2 | 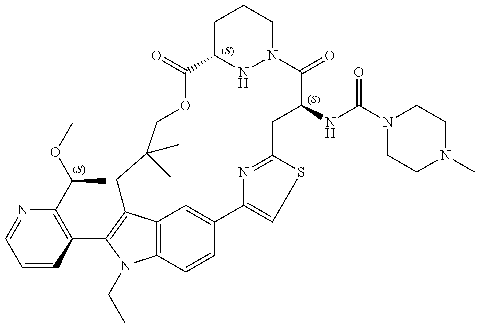 |
| DC3 | 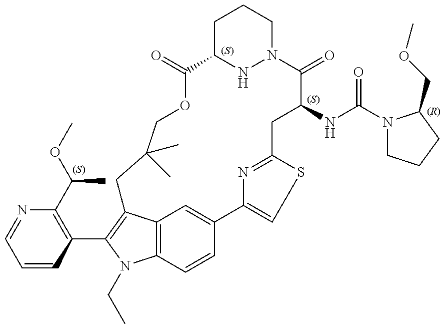 |
| DC4 | 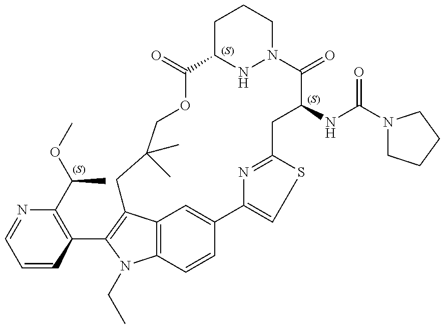 |
| DC5 | 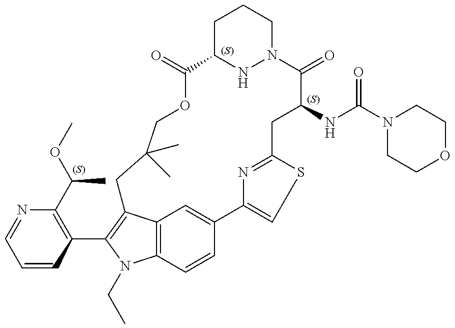 |
| DC6 | 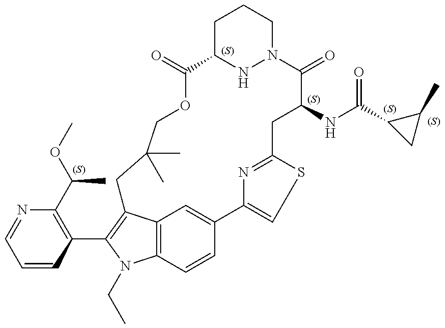 |
| DC7 | 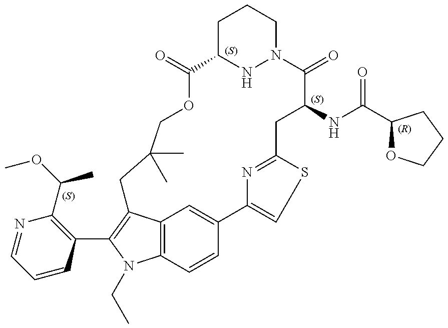 |
| DC8 | 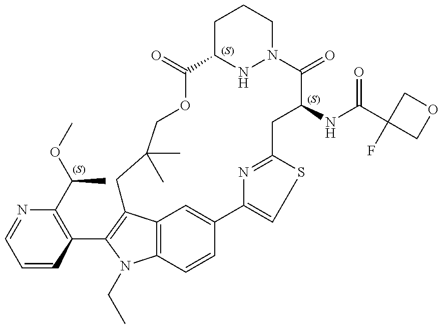 |
| DC9 | 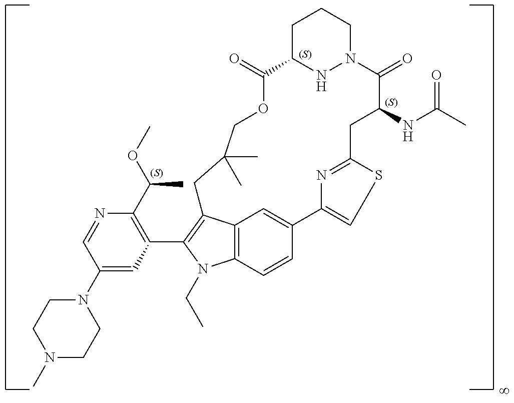 |
| DC10 | 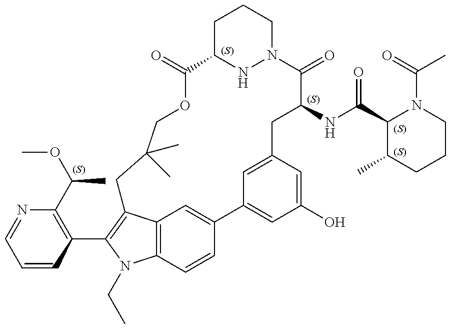 |
| DC11 | 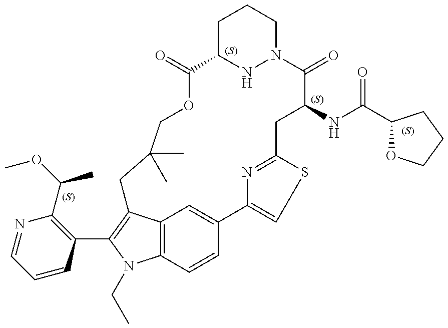 |
| DC12 | 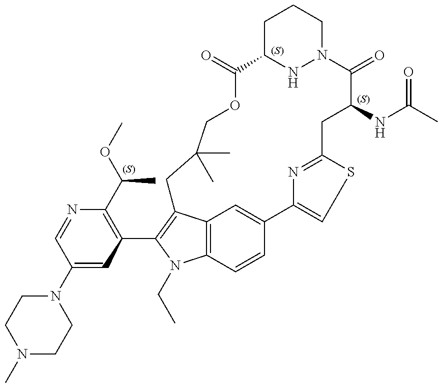 |
| DC13 | 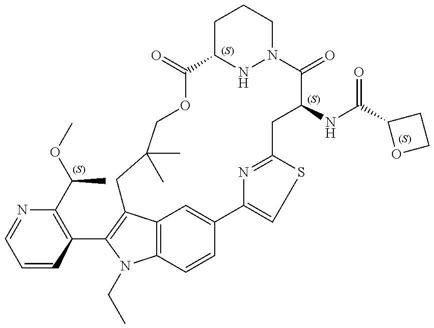 |
| DC14 | 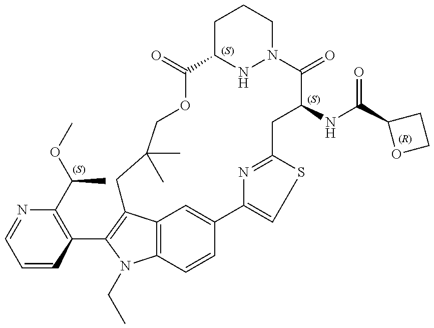 |
| DC15 | 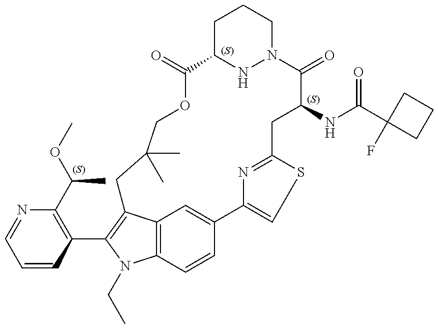 |
| DC16 | 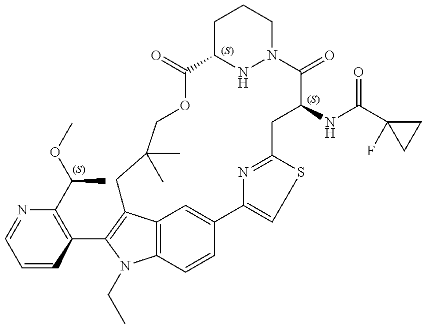 |
| DC17 | 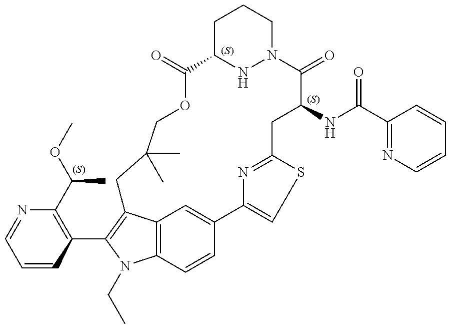 |
| DC18 | 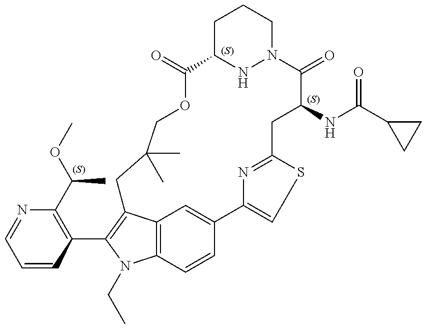 |
| DC19 | 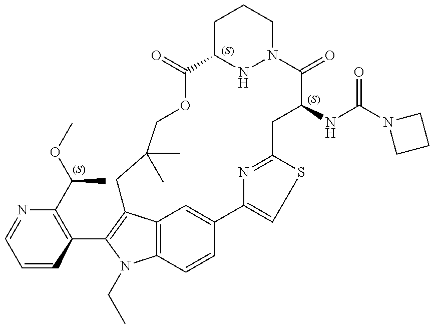 |
| DC20 | 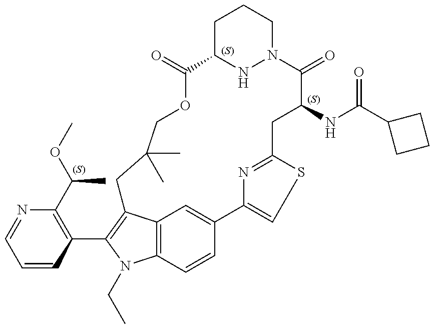 |
| DC21 | 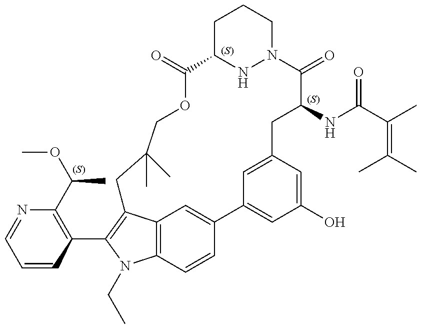 |

The compounds described herein in Tables D1a, D1b, D2, and D3 may be made from commercially available starting materials or synthesized using known organic, inorganic, or enzymatic processes.

The compounds of the present invention in Tables D1a, D1b, D2, and D3 can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below and in WO 2022/060836.

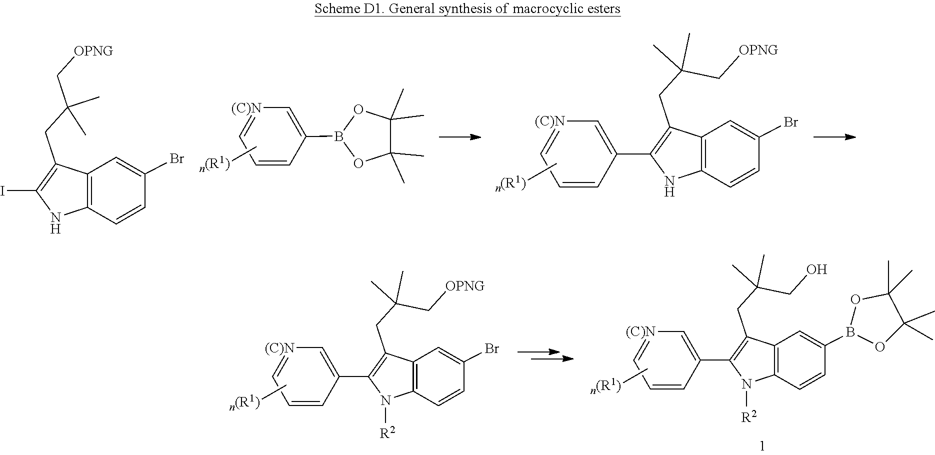

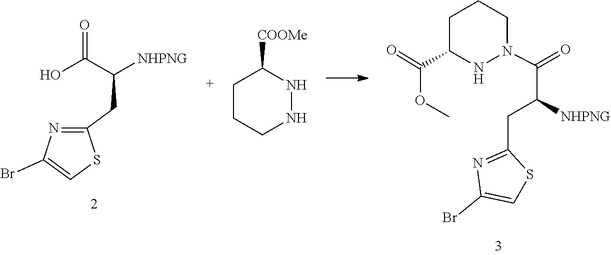

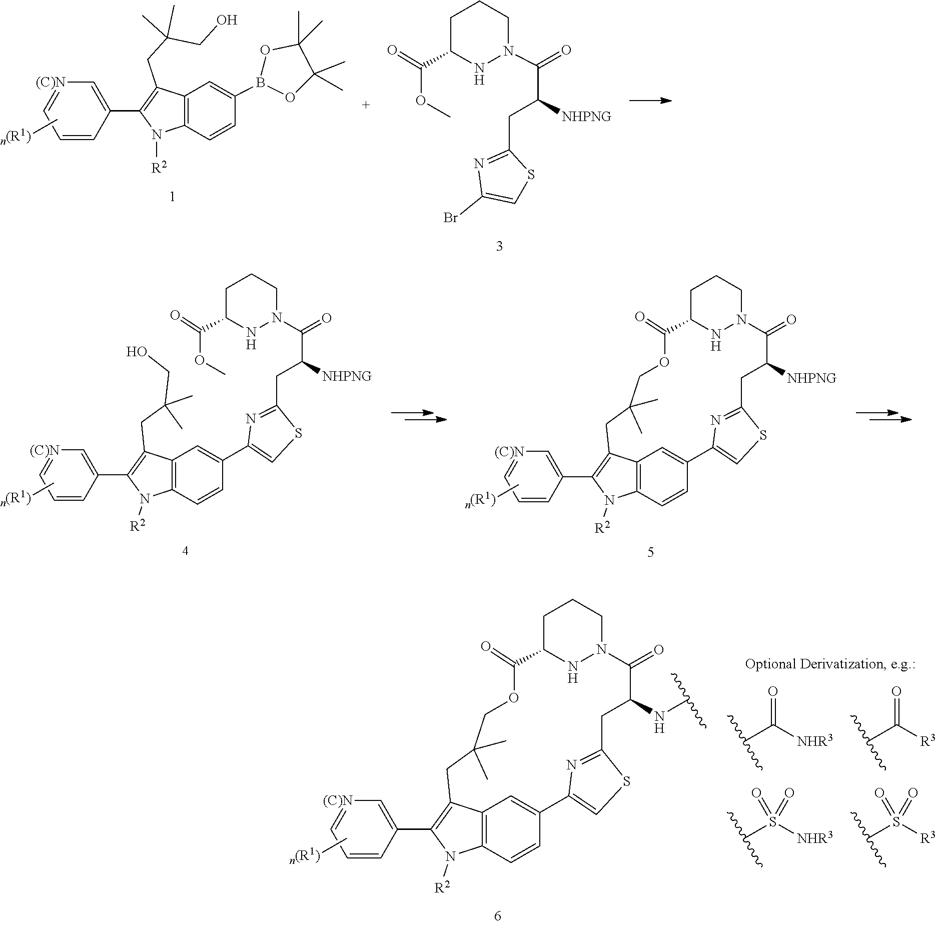

A general synthesis of macrocyclic esters is outlined in Scheme D1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl)propanoyl)hexahydropyridazine-3-carboxylate (3) can be prepared via coupling of (S)-2-amino-3-(4-bromothiazol-2-yl)propanoic acid (2) with methyl (S)-hexahydropyridazine-3-carboxylate.

The final macrocyclic esters can be made by coupling of methyl-amino-3-(4-bromothiazol-2-yl)propanoyl)hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) can result in a macrocyclic product. Additional deprotection or functionalization steps could be required to produce a final compound 6.

Further, with respect to Scheme D1, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

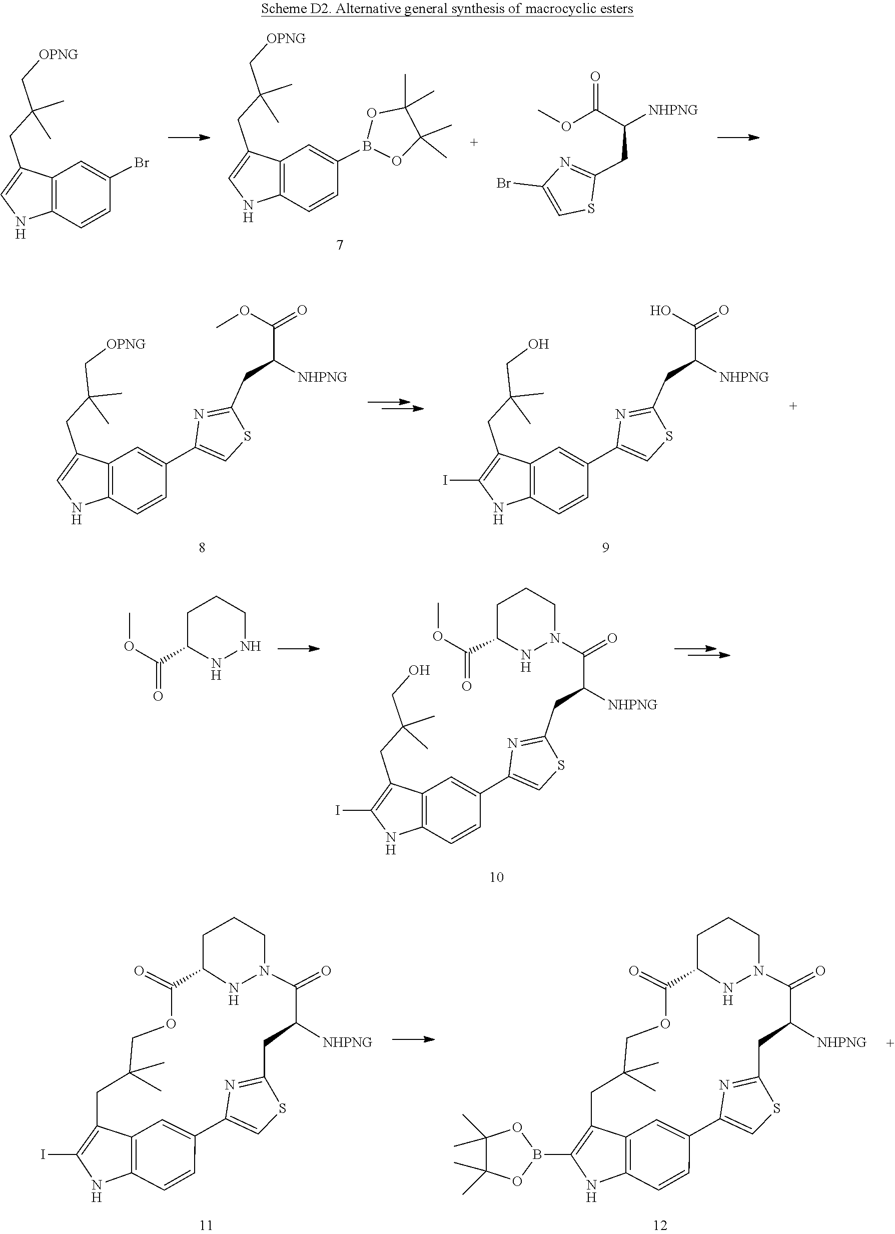

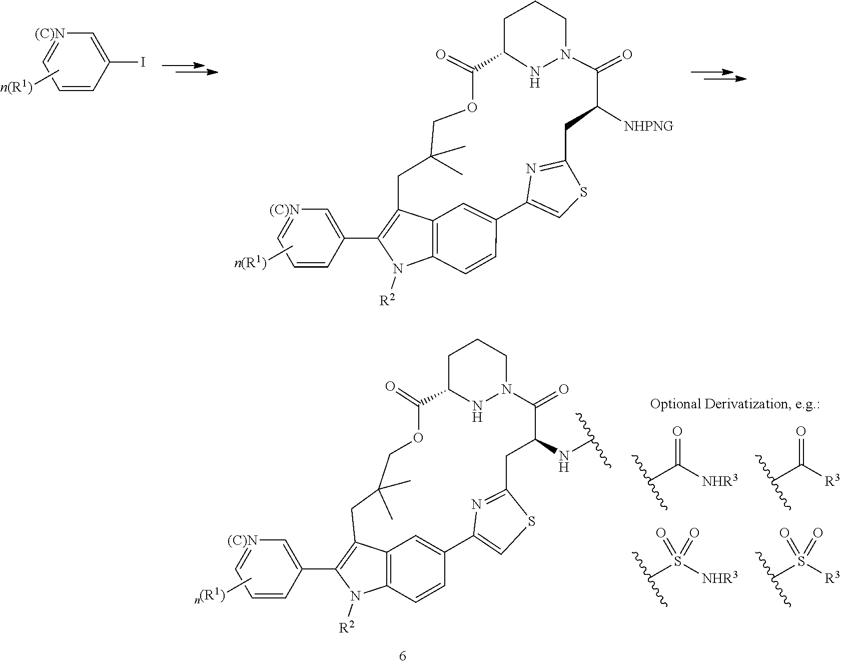

Alternatively, macrocyclic esters can be prepared as described in Scheme D2. An appropriately substituted and protected indolyl boronic ester (7) can be coupled in the presence of Pd catalyst with (S)-2-amino-3-(4-bromothiazol-2-yl)propanoic acid, followed by iodination, deprotection, and ester hydrolysis. Subsequent coupling with methyl (S)-hexahydropyridazine-3-carboxylate, followed by hydrolysis and macrolactonization can result in iodo intermediate (11). Subsequent palladium mediated borylation and coupling in the presence of Pd catalyst with an appropriately substituted iodo aryl or iodo heteroaryl intermediate can yield an appropriately protected macrocyclic intermediate. Alkylation, deprotection and coupling with an appropriately substituted carboxylic acid carboxylic acid (or other coupling partner) results in a macrocyclic product. Additional deprotection or functionalization steps could be required to produce a final compound 6.

Further, with respect to Scheme D2, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

Compounds of Table D1a or Table D1b herein were prepared using methods disclosed in WO 2022/060836 or were prepared using methods described herein combined with the knowledge of one of skill in the art.

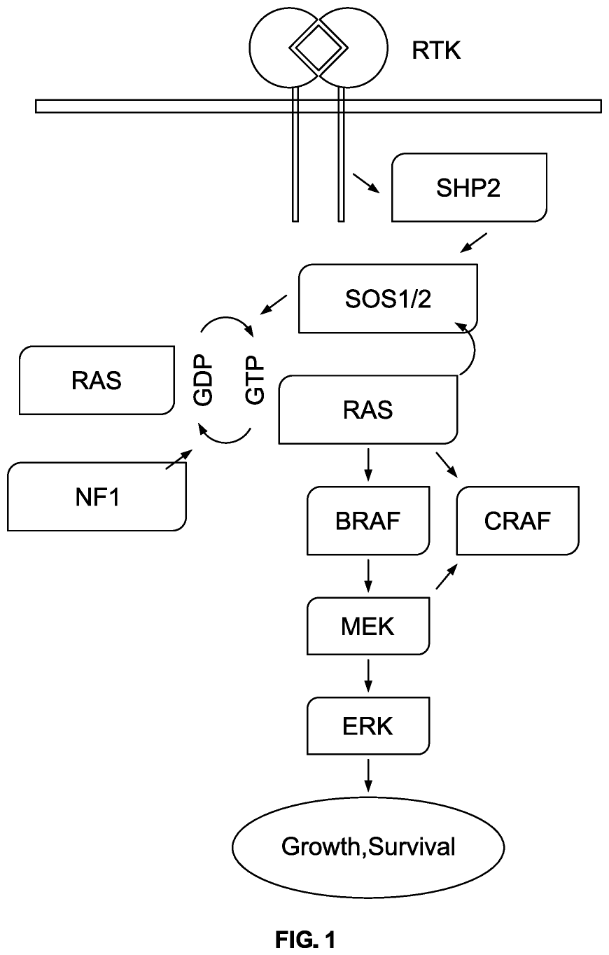

In some embodiments, the RAS(ON) inhibitor is a compound described by a Formula in WO 2020132597, such as a compound of FIG. 1 therein, or a pharmaceutically acceptable salt thereof.

In some embodiments, the RAS(ON) inhibitor is RM-018. “RM-018,” as referred to herein, mean the following compound:

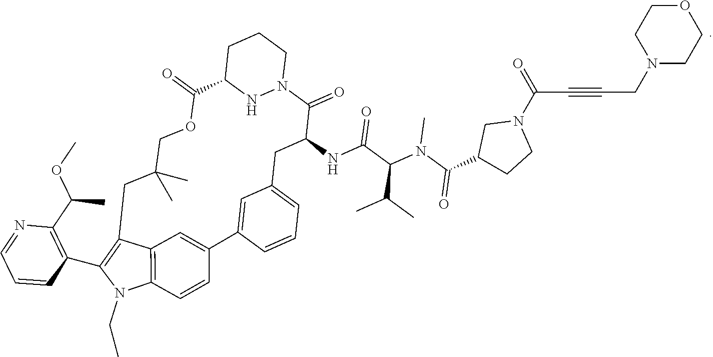

In some embodiments, a RAS(ON) inhibitor described herein entails formation of a high affinity three-component complex between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the RAS(ON) inhibitors described herein induce a new binding pocket in RAS by driving formation of a high affinity tri-complex between the RAS protein and the widely expressed cytosolic chaperone, cyclophilin A (CYPA). Without being bound by theory, one way the inhibitory effect on Ras is affected by compounds of the invention and the complexes they form is by steric occlusion of the interaction site between Ras and downstream effector molecules, such as RAF and PI3K, which are required for propagating the oncogenic signal.

Without being bound by theory, both covalent and non-covalent interactions of a RAS(ON) inhibitor described herein with Ras and the chaperone protein (e.g., cyclophilin A) may contribute to the inhibition of Ras activity. In some embodiments, a RAS(ON) inhibitor described herein forms a covalent adduct with a side chain of a Ras protein (e.g., a sulfhydryl side chain of the cysteine at position 12 or 13 of a mutant Ras protein). Covalent adducts may also be formed with other side chains of Ras. In addition, or alternatively, non-covalent interactions may be at play: for example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by RAS(ON) inhibitors described herein (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art and are described in, for example, WO 2021/091982 and WO 2021/091967.

In some embodiments, the RAS inhibitor is selected from the group consisting of Compound RAS-(A), Compound RAS-(B), Compound RAS-(C), Compound RAS-(D), Compound RAS-(E), Compound RAS-(F), and any combination thereof. It is to be understood that any one of Compound RAS-(A), Compound RAS-(B), Compound RAS-(C), Compound RAS-(D), Compound RAS-(E), and Compound RAS-(F) could be found in any one of WO 2021/091956, WO 2021/091982, WO 2021/091967, and WO 2022/060836. Accordingly, the letter reference to the RAS compound (e.g., RAS-(A)) should not be understood to necessarily indicate that the compound can be found in the corresponding WO 2021/091956.

In some embodiments, the RAS inhibitor is Compound RAS-(A), or a pharmaceutically acceptable salt, solvate, isomer, prodrug, or tautomer thereof. Compound RAS-(A) is a compound falling within the scope of Formula BI.

In some embodiments, the RAS inhibitor is Compound RAS-(B), or a pharmaceutically acceptable salt, solvate, isomer, prodrug, or tautomer thereof. Compound RAS-(B) is a compound falling within the scope of Formula BI.

In some embodiments, the RAS inhibitor is Compound RAS-(C), or a pharmaceutically acceptable salt, solvate, isomer, prodrug, or tautomer thereof. Compound RAS-(C) is a compound falling within the scope of Formula BI.

In some embodiments, the RAS inhibitor is Compound RAS-(D), or a pharmaceutically acceptable salt, solvate, isomer, prodrug, or tautomer thereof. Compound RAS-(D) is a compound falling within the scope of Formula A00.

In some embodiments, the RAS inhibitor is Compound RAS-(E), or a pharmaceutically acceptable salt, solvate, isomer, prodrug, or tautomer thereof. Compound RAS-(E) is a compound falling within the scope of Formula DI.

In some embodiments, the RAS inhibitor is Compound RAS-(F), or a pharmaceutically acceptable salt, solvate, isomer, prodrug, or tautomer thereof. Compound RAS-(F) is a compound falling within the scope of Formula BI.

In some embodiments, the RAS inhibitor is selective for a mutation at position 12 or 13 of a RAS protein. In some embodiments, the RAS inhibitor selectively targets RAS G12D. In some embodiments, the RAS inhibitor is MRTX1133.

The RAS inhibitor dose may range from a dose sufficient to elicit a response to the maximum tolerated dose. Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses, such as from 100 mg to 1300 mg, from 200 mg to 1300 mg, from 600 mg to 1300 mg, from 700 mg to 1200 mg, or from 800 mg to 1000 mg. In one embodiment, the compositions are in the form of a tablet that can be scored. The RAS inhibitor can be dosed once per day, twice per day, three times per day, or four times per day. In some aspects, RAS inhibitor is dosed once per day. In some aspects, RAS inhibitor is dosed twice per day. Dosing may be done with or without food. The dosing schedule may suitably be every day of a 28-day schedule, or 21 or more days of a 28-day schedule.

### RAS(OFF) Inhibitors

RAS(OFF) inhibitors are provided herein and are known to those of skill in the art. A RAS(OFF) inhibitor refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

In some embodiments, the RAS(OFF) inhibitor is selective for RAS that includes an amino acid substitution at G12, G13, Q61, or a combination thereof. In some embodiments, the RAS(OFF) inhibitor is selective for RAS that includes an amino acid substitution selected from G12C, G12D, G12V, G13C, G13D, Q61L, or a combination thereof. In some embodiments, the RAS(OFF) inhibitor is selective for RAS that includes a G12C or G12D amino acid substitution.

In some embodiments, the RAS(OFF) inhibitor is a KRAS(OFF) inhibitor, where a KRAS(OFF) inhibitor refers to an inhibitor that targets, that is, selectively binds to or inhibits the GDP-bound, inactive state of KRAS (e.g., selective over the GTP-bound, active state of KRAS). In some embodiments, the KRAS(OFF) inhibitor is selective for KRAS that includes an amino acid substitution at G12, G13, Q61, A146, K117, L19, Q22, V14, A59, or a combination thereof. In some embodiments, the KRAS(OFF) inhibitor is selective for KRAS that includes an amino acid substitution selected from G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, G13V, or a combination thereof.

In some embodiments, the RAS(OFF) inhibitor is an NRAS(OFF) inhibitor, where an NRAS(OFF) inhibitor refers to an inhibitor that targets, that is, selectively binds to or inhibits the GDP-bound, inactive state of NRAS (e.g., selective over the GTP-bound, active state of NRAS). In some embodiments, the NRAS(OFF) inhibitor is selective for NRAS that includes an amino acid substitution at G12, G13, Q61, P185, A146, G60, A59, E132, E49, T50, or a combination thereof. In some embodiments, the NRAS(OFF) inhibitor is selective for NRAS that includes an amino acid substitution selected from Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, A59T, or a combination thereof.

In some embodiments, the RAS(OFF) inhibitor is an HRAS(OFF) inhibitor, where an HRAS(OFF) inhibitor refers to an inhibitor that targets, that is, selectively binds to or inhibits the GDP-bound, inactive state of HRAS (e.g., selective over the GTP-bound, active state of HRAS). In some embodiments, the HRAS(OFF) inhibitor is selective for HRAS that includes an amino acid substitution at G12, G13, Q61, K117, A59, A18, D119, A66, A146, or a combination thereof. In some embodiments, the HRAS(OFF) inhibitor is selective for NRAS that includes an amino acid substitution selected from Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, G12R, or a combination thereof.

In some embodiments, the RAS(OFF) inhibitor is a compound disclosed in any one of the following patent publications: WO 2022066805, WO 2022066646, WO 2022063297, WO 2022061251, WO 2022056307, WO 2022052895, WO 2022047093, WO 2022042630, WO 2022040469, WO 2022037560, WO 2022031678, WO 2022028492, WO 2022028346, WO 2022026726, WO 2022026723, WO 2022015375, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223, each of which is incorporated herein by reference in its entirety.

In some embodiments, the RAS(OFF) inhibitor is selected from AMG 510 (sotorasib), MRTX849 (adagrasib), MRTX1257, JNJ-74699157 (ARS-3248), LY3537982, LY3499446, ARS-853, ARS-1620, GDC-6036, and JDQ443.

In any embodiment herein regarding a RAS(OFF) inhibitor, the RAS(OFF) inhibitor may be substituted by a RAS inhibitor disclosed in the following patent publication: WO 2021041671, which is incorporated herein by reference in its entirety. In some embodiments, such a substituted RAS inhibitor is MRTX1133.

### Exemplary Combinations of RAS Inhibitors and SOS1 Inhibitors

In some embodiments, the method comprises administering a combination of a RAS inhibitor and a SOS1 inhibitor. Exemplary, non-limiting combinations of such inhibitors include the following.

In one embodiment, (a) the SOS1 inhibitor is Compound SOS1-(A), having the structure:

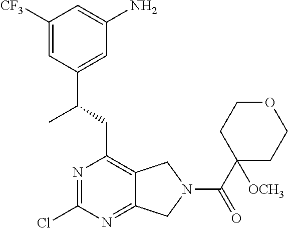 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, prodrug, or tautomer thereof, and (b) the RAS inhibitor is Compound RAS-(C), or a pharmaceutically acceptable salt, solvate, isomer, prodrug, or tautomer thereof.

In one embodiment, (a) the SOS1 inhibitor is Compound SOS1-(A), having the structure:

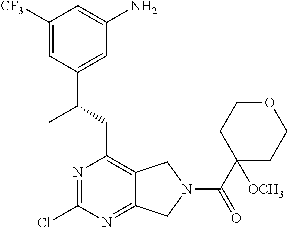 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, prodrug, or tautomer thereof, and (b) the RAS inhibitor is Compound RAS-(D), or a pharmaceutically acceptable salt, solvate, isomer, prodrug, or tautomer thereof.

In one embodiment, (a) the SOS1 inhibitor is Compound SOS1-(B), or a pharmaceutically acceptable salt, solvate, isomer, prodrug, or tautomer thereof; and (b) the RAS inhibitor is Compound RAS-(B), or a pharmaceutically acceptable salt, solvate, isomer, prodrug, or tautomer thereof.

In one embodiment, (a) the SOS1 inhibitor is Compound SOS1-(B), or a pharmaceutically acceptable salt, solvate, isomer, prodrug, or tautomer thereof; and (b) the RAS inhibitor is Compound RAS-(E), or a pharmaceutically acceptable salt, solvate, isomer, prodrug, or tautomer thereof.

In one embodiment, (a) the SOS1 inhibitor is Compound SOS1-(C), or a pharmaceutically acceptable salt, solvate, isomer, prodrug, or tautomer thereof; and (b) the RAS inhibitor is Compound RAS-(F), or a pharmaceutically acceptable salt, solvate, isomer, prodrug, or tautomer thereof.

In one embodiment, (a) the SOS1 inhibitor is BI-3406,
or a pharmaceutically acceptable salt, solvate, isomer, prodrug, or tautomer thereof; and (b) the RAS inhibitor is Compound RAS-(A), or a pharmaceutically acceptable salt, solvate, isomer, prodrug, or tautomer thereof.

In one embodiment, (a) the SOS1 inhibitor is BI-3406, or a pharmaceutically acceptable salt, solvate, isomer, prodrug, or tautomer thereof; and (b) the RAS inhibitor is Compound RAS-(C), or a pharmaceutically acceptable salt, solvate, isomer, prodrug, or tautomer thereof.

In some embodiments, (a) the SOS1 inhibitor is BI-1701963 or a pharmaceutically acceptable salt, solvate, isomer, prodrug, or tautomer thereof; and (b) the RAS inhibitor is selected from the group consisting of Compound RAS-(A), Compound RAS-(B), Compound RAS-(C), Compound RAS-(D), Compound RAS-(E), and any combination thereof, or a pharmaceutically acceptable salt, solvate, isomer, prodrug, or tautomer of any of the above.

In some embodiments, (a) the SOS1 inhibitor is BI-1701963 or a pharmaceutically acceptable salt, solvate, isomer, prodrug, or tautomer thereof; and (b) the RAS inhibitor is Compound RAS-(A), or a pharmaceutically acceptable salt, solvate, isomer, prodrug, or tautomer thereof.

In some embodiments, (a) the SOS1 inhibitor is BI-1701963 or a pharmaceutically acceptable salt, solvate, isomer, prodrug, or tautomer thereof; and (b) the RAS inhibitor is Compound RAS-(B), or a pharmaceutically acceptable salt, solvate, isomer, prodrug, or tautomer thereof.

In some embodiments, (a) the SOS1 inhibitor is BI-1701963 or a pharmaceutically acceptable salt, solvate, isomer, prodrug, or tautomer thereof; and (b) the RAS inhibitor is Compound RAS-(C), or a pharmaceutically acceptable salt, solvate, isomer, prodrug, or tautomer thereof.

In some embodiments, (a) the SOS1 inhibitor is BI-1701963 or a pharmaceutically acceptable salt, solvate, isomer, prodrug, or tautomer thereof; and (b) the RAS inhibitor is Compound RAS-(D), or a pharmaceutically acceptable salt, solvate, isomer, prodrug, or tautomer thereof.

In some embodiments, (a) the SOS1 inhibitor is BI-1701963 or a pharmaceutically acceptable salt, solvate, isomer, prodrug, or tautomer thereof; and (b) the RAS inhibitor is Compound RAS-(E), or a pharmaceutically acceptable salt, solvate, isomer, prodrug, or tautomer thereof.

### Additional Combination Therapies

In some embodiments, the subject is co-administered a therapeutically effective amount of an additional therapeutic agent.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MED14736, MPDL3280A, MSB0010718C, BMS986016, IMP321, lirilumab, IPH2101, 1-7F9, and KW-6002.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antibiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK 4/6 inhibitor such as ribociclib, abemaciclib, or palbociclib), seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CS1 (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, the anti-cancer agent is a colony-stimulating factor 1 receptor (CSF1R) inhibitor. See, e.g., Cannarile et al., J Immuno Therapy Cancer 5:53 (2017) and Xun et al., Curr Med Chem 27:3944 (2020).

In some embodiments, an anti-cancer agent is an anti-CD40 antibody, such as APX005M.

A therapeutic agent may be an anti-TIGIT antibody, such as MBSA43, BMS-986207, MK-7684, COM902, AB154, MTIG7192A or OMP-313M32 (etigilimab).

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, ERAS-601, SH3809, PF-07284892, or BBP-398)), another SOS1 inhibitor (e.g., BI-1701963, BI-3406, SDR5, or BAY-293), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312. In some embodiments, an anti-cancer agent is a Ras inhibitor (e.g., AMG 510, MRTX1257, JNJ-74699157 (ARS-3248), LY3537982, ARS-853, ARS-1620, GDC-6036, RMC-6236, RMC-6291, RMC-8839, RMC-9805, BPI-421286, JDQ443, or JAB-21000, or a Ras vaccine, or another therapeutic modality designed to directly or indirectly decrease the oncogenic activity of Ras.

In some embodiments, a therapeutic agent is an inhibitor of the MAP kinase (MAPK) pathway (or “MAPK inhibitor”). MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, R04987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000).

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res.1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa@), erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is osimertinib.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; AF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl] phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Akt1) (Barnett et al., Biochem. J. 2005, 385(Pt. 2): 399-408); Akt-1-1,2 (inhibits Ak1 and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g., AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAG1, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-1a1fa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™) bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

In some embodiments, an additional compound is selected from the group consisting of a CDK4/6 inhibitor (e.g., abemaciclib, palbociclib, or ribociclib), a KRAS:GDP G12C inhibitor (e.g., AMG 510, MRTX 1257) or other mutant Ras:GDP inhibitor, a KRAS:GTP G12C inhibitor or other mutant Ras:GTP inhibitor, a MEK inhibitor (e.g., refametinib, selumetinib, trametinib, or cobimetinib), a SHP2 inhibitor (e.g., TNO155, RMC-4630), an ERK inhibitor, and an RTK inhibitor (e.g., an EGFR inhibitor). In some embodiments, a SOS1 inhibitor may be used in combination with a Ras inhibitor, a SHP2 inhibitor, or a MEK inhibitor. In some embodiments, a combination therapy includes a SOS1 inhibitor, a RAS inhibitor and a MEK inhibitor.

In some embodiments, an additional compound is selected from the group consisting of ABT-737, AT-7519, carfilzomib, cobimetinib, danusertib, dasatinib, doxorubicin, GSK-343, JQ1, MLN-7243, NVP-ADW742, paclitaxel, palbociclib and volasertib. In some embodiments, an additional compound is selected from the group consisting of neratinib, acetinib and reversine.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In the above, preferred additional therapeutic agents include MEK inhibitors, ERK inhibitors, pan-RAS(ON) inhibitors (that is, inhibitors that target the GTP-activated form of RAS), CDK4/6 inhibitors, mTORC1 inhibitors, HDAC inhibitors, BCL2 inhibitors, and PLK1 inhibitors.

### EMBODIMENTS

Embodiment 1 is a method of treating a subject having a RAS protein-related disease or disorder, the method comprising administering to a subject in need of such treatment:
(a) a therapeutically effective amount of a SOS1 inhibitor having the structure of Formula (41-I),

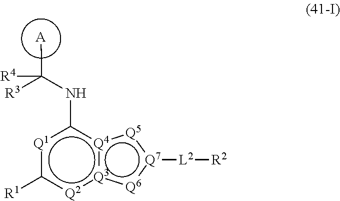

or a pharmaceutically acceptable salt, solvate, isomer, prodrug, or tautomer thereof, wherein:
Q¹ and Q² are independently CH or N;
Q³, Q⁴, and Q⁷ are independently C or N, wherein at least one of Q³ and Q⁴ is C and wherein Q³, Q⁴, and Q⁷ are not all N;
Q⁵ is CH, N, NH, O, or S;
Q⁶ is CH, N, NH, N—C₁₋₆ alkyl, N—C₁₋₆ heteroalkyl, N-(3-7 membered cycloalkyl), N-(3-7 membered heterocyclyl), O, or S;
wherein at least one of Q¹, Q², Q³, Q⁴, Q⁵, Q⁶, and Q⁷ is N, NH, O, or S;
R¹ is selected from the group consisting of H, C₁₋₆ alkyl, halogen, —NHR^{1a}, —OR^{1a} cyclopropyl, and —CN; wherein C₁₋₆ alkyl is optionally substituted with halogen, —NHR^{1a} or —OR^{1a}; wherein R^{1a} is H, C₁₋₆ alkyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

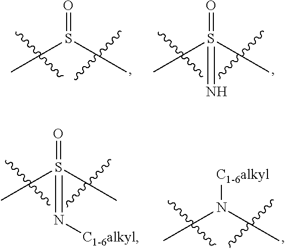

—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, C₂₋₆ alkenyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, C₂₋₆ alkenyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, —OH, —OR^{2a}, oxo, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl; and
(b) a therapeutically effective amount of a RAS inhibitor selected from the group consisting of a RAS(ON) inhibitor and a RAS(OFF) inhibitor, and a combination thereof.

Embodiment 2 is a method of treating a subject having a RAS protein-related disease or disorder, the method comprising administering to a subject in need of such treatment:
(a) a therapeutically effective amount of a SOS1 inhibitor having the structure of Formula (42-I),

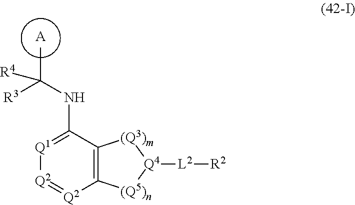

or a pharmaceutically acceptable salt, solvate, isomer, prodrug, or tautomer thereof, wherein:
Q¹ is CH orN;
Q⁴ is CH, C, or N;
each Q² is independently C—R¹ or N, wherein one Q² is N and the other Q² is C—R¹;
each Q³ and Q⁵ are independently C(R^{QC})₂, NR^{QN}, CO, O, S, or SO₂, wherein each R^{QC} is independently H, F, Cl, Br, or 6-10 membered aryl, and wherein each R^{QN} is independently H, C₁₋₆ alkyl, or 6-10 membered aryl; wherein at least one of Q¹, Q², Q³, Q⁴, and Q⁵ is N, NR^{QN}, O, or SO₂;
m is 0, 1, 2, or 3;
n is 0, 1, 2, or 3;
wherein when m is 0, then n is not 0;
R¹ is selected from the group consisting of H, C₁₋₆ alkyl, halogen, —CONHR^{1a}, —
NHR^{1a}—OR^{1a}, cyclopropyl, azetidinyl, and —CN; wherein each C₁₋₆ alkyl and azetidinyl is optionally substituted with halogen, R^{1a}, —NHR^{1a} or —OR^{1a}; wherein R^{1a} is H, C₁₋₆ alkyl, cyclopropyl, 3-6 membered heterocyclyl, or C₁₋₆ haloalkyl;
L² is selected from the group consisting of a bond, —C(O)—, —C(O)O—, —C(O)NH(CH₂)ₒ—, —S(O)₂—,

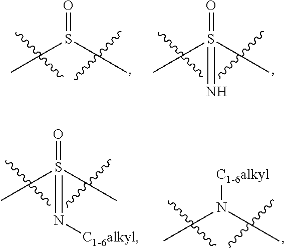

—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
   wherein R^{2a} is H, C₁₋₆ alkyl, C₁₋₆ haloalkyl, 3-7 membered heterocyclyl, or —(CH₂)ᵣOCH₃, wherein r is 1, 2, or 3;
   wherein R^{2b} is H or C₁₋₆ alkyl;
   wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl; and
(b) a therapeutically effective amount of a RAS inhibitor selected from the group consisting of a RAS(ON) inhibitor and a RAS(OFF) inhibitor, and a combination thereof.

In some embodiments of Embodiment 2, the compound is characterized by the proviso that when

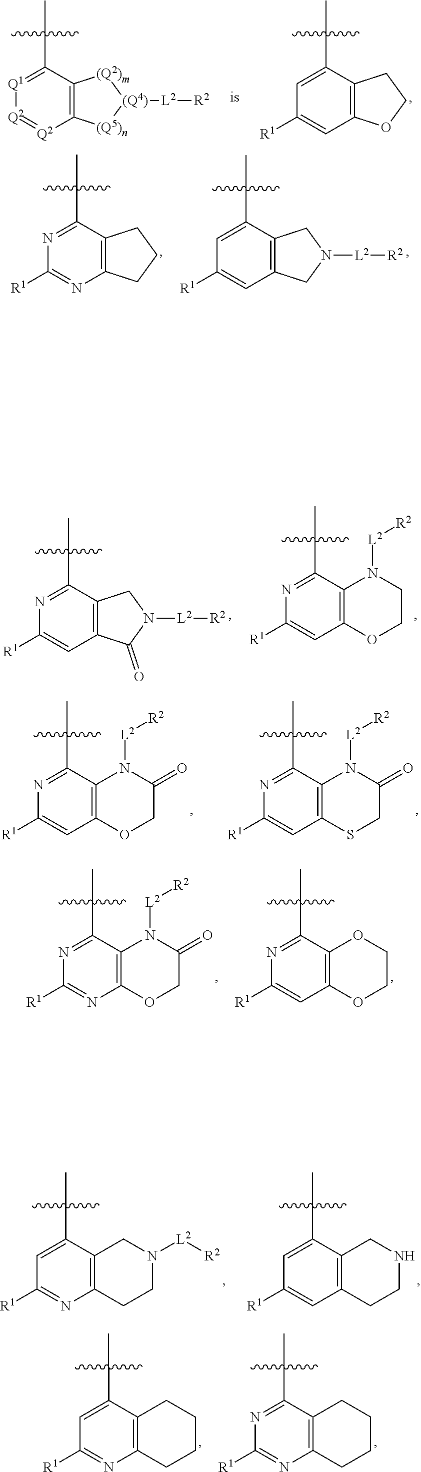

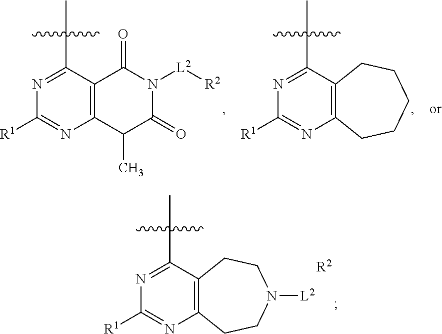

then R is not H.

Embodiment 3 is a method of Embodiment 2, wherein the SOS1 inhibitor is Compound SOS1-(A) (also called RMC-0331), having the structure:

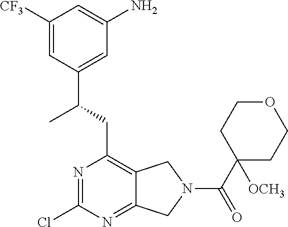 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, prodrug, or tautomer thereof.

Embodiment 4 is a method of treating a subject having a RAS protein-related disease or disorder, the method comprising administering to a subject in need of such treatment:
(a) a therapeutically effective amount of a SOS1 inhibitor having the structure of Formula (48-I),

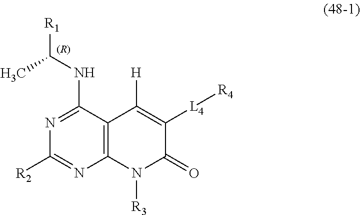

or a pharmaceutically acceptable salt, solvate, isomer, prodrug, or tautomer thereof, wherein:
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocyclyl, and further wherein R₂ₐ is selected from the group consisting of H, C₁₋₆ alkyl, 3-6 membered heterocyclyl, and C₁₋₆ haloalkyl;
R₃ is selected from the group consisting of H, C₁₋₃ alkyl, —OR₃ₐ, cyclopropyl, and 3-6 membered heterocyclyl, wherein each of C₁₋₃ alkyl, cyclopropyl, and 3-6 membered heterocyclyl is optionally substituted with R₃a, and further wherein R₃a is selected from the group consisting of C₁₋₃ alkyl, halogen, —OH, and —CN;
L₄ is selected from the group consisting of bond, —C(O)—, —C(O)O—, —C(O)NH(CH₂)ₒ—, —NH—, —S—, —S(O)₂—,

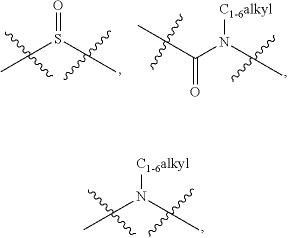

—(CH₂)ₚ—, and —O—; wherein o is 0, 1, or 2; and wherein p is a number from 1 to 6; and
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl, 3-7 membered heterocyclyl, 6-10 membered aryl, or 5-10 membered heteroaryl;
   wherein R₄a is H, C₁₋₆ alkyl, C₁₋₆ haloalkyl, —C(O)NR_{4b}R_{4c}, 3-6 membered cycloalkyl, 6-10 membered aryl optionally substituted with —OR_{4b}, —CN, 3-7 membered heterocyclyl, —(CH₂)ᵣOCH₃, or —(CH₂)ᵣOH, wherein r is 1, 2, or 3;
   wherein each R_{4b} is independently H, C₁₋₆ alkyl; and
   wherein each R_{4c} is independently H or C₁₋₆ alkyl; and
(b) a therapeutically effective amount of a RAS inhibitor selected from the group consisting of a RAS(ON) inhibitor and a RAS(OFF) inhibitor, and a combination thereof.

Embodiment 5 is a method of treating a subject having a RAS protein-related disease or disorder, the method comprising administering to a subject in need of such treatment:
(a) a therapeutically effective amount of a SOS1 inhibitor, wherein the SOS1 inhibitor is BI-3406, having the structure:

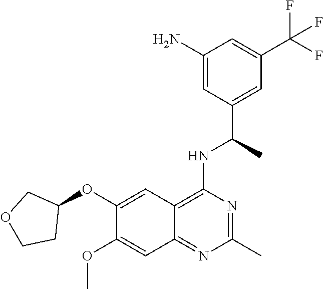 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer, prodrug, or tautomer thereof, and
(b) a therapeutically effective amount of a RAS inhibitor selected from the group consisting of a RAS(ON) inhibitor and a RAS(OFF) inhibitor, and a combination thereof.

Embodiment 6 is a method of treating a subject having a RAS protein-related disease or disorder, the method comprising administering to a subject in need of such treatment:
(a) a therapeutically effective amount of a SOS1 inhibitor, wherein the SOS1 inhibitor is BI-1701963, or a pharmaceutically acceptable salt, solvate, isomer, prodrug, or tautomer thereof, and
(b) a therapeutically effective amount of a RAS inhibitor selected from the group consisting of a RAS(ON) inhibitor and a RAS(OFF) inhibitor, and a combination thereof.

Embodiment 7 is a method of any one of Embodiments 1 through 6, wherein the RAS inhibitor is selective for a mutation at position 12 or 13 of a RAS protein.

Embodiment 8 is a method of any one of Embodiments 1 through 7, wherein the RAS inhibitor is a RAS(ON) inhibitor.

Embodiment 9 is a method of Embodiment 8, wherein the RAS(ON) inhibitor is an inhibitor selective for RAS G12C, RAS G13D, or RAS G12D.

Embodiment 10 is a method of Embodiment 8, wherein the RAS(ON) inhibitor is a RAS(ON)^{MULTI} inhibitor.

Embodiment 11 is a method of Embodiment 8, wherein the RAS(ON) inhibitor is a compound described by Formula A00, Formula A1, Formula BI, Formula CI, Formula DIa, and subformula thereof, and compounds of Table A1, Table A2, Table B1, Table B2, Table C1, Table C2, Table D1a, Table D1b, Table D2, Table D3, and pharmaceutically acceptable salts, solvates, hydrates, stereoisomers, and tautomers thereof.

Embodiment 12 is a method of Embodiment 11, wherein the RAS(ON) inhibitor is a compound described by Formula A00, or Formula A1, or a subformula thereof, or a pharmaceutically acceptable salt thereof.

Embodiment 13 is a method of Embodiment 12, wherein the RAS(ON) inhibitor is selected from a compound of Table A1 or Table A2, or a pharmaceutically acceptable salt thereof.

Embodiment 14 is a method of Embodiment 11, wherein the RAS(ON) inhibitor is a compound described by Formula BI, or a subformula thereof, or a pharmaceutically acceptable salt thereof.

Embodiment 15 is a method of Embodiment 14, wherein the RAS(ON) inhibitor is selected from a compound of Table B1 or Table B2, or a pharmaceutically acceptable salt thereof.

Embodiment 16 is a method of Embodiment 11, wherein the RAS(ON) inhibitor is a compound described by Formula CI, or a subformula thereof, or a pharmaceutically acceptable salt thereof.

Embodiment 17 is a method of Embodiment 16, wherein the RAS(ON) inhibitor is selected from a compound of Table C1 or Table C2, or a pharmaceutically acceptable salt thereof.

Embodiment 18 is a method of Embodiment 11, wherein the RAS(ON) inhibitor is a compound described by Formula DIa, or a subformula thereof, or a pharmaceutically acceptable salt thereof.

Embodiment 19 is a method of Embodiment 18, wherein the RAS(ON) inhibitor is selected from a compound of Table D1a, Table D1b, Table D2, Table D3, or a pharmaceutically acceptable salt thereof.

Embodiment 20 is a method of Embodiment 8, wherein the RAS(ON) inhibitor is selected from the group consisting of RAS-(A), RAS-(B), RAS-(C), RAS-(D), RAS-(E), RAS-(F), and any combination thereof.

Embodiment 21 is a method of any one of Embodiments 1 through 7, wherein the RAS inhibitor is a RAS(OFF) inhibitor.

Embodiment 22 is a method of Embodiment 21, wherein the RAS(OFF) inhibitor selectively targets RAS G12C.

Embodiment 23 is a method of Embodiment 21, wherein the RAS(OFF) inhibitor is selected from sotorasib (AMG 510), adagrasib (MRTX849), MRTX1257, JNJ-74699157 (ARS-3248), LY3537982, ARS-853, ARS-1620, GDC-6036, BPI-421286, JDQ443, and JAB-21000.

Embodiment 24 is a method of any one of Embodiments 1 through 7, wherein the RAS inhibitor selectively targets RAS G12D.

Embodiment 25 is a method of Embodiment 24, wherein the RAS inhibitor is MRTX1133.

Embodiment 26 is a method of Embodiment 2, wherein:
(a) the SOS1 inhibitor is Compound SOS1-(A) (also called RMC-0331), having the structure:

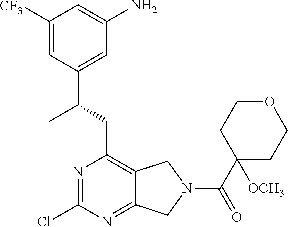 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, prodrug, or tautomer thereof, and
(b) the RAS inhibitor is Compound RAS-(C), or a pharmaceutically acceptable salt, solvate, isomer, prodrug, or tautomer thereof.

Embodiment 27 is a method of Embodiment 2, wherein:
(a) the SOS1 inhibitor is Compound SOS1-(A) (also called RMC-0331), having the structure:

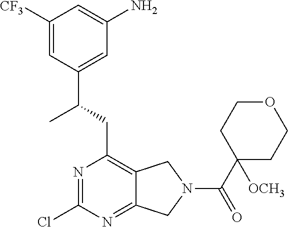 [SMILES: COC1(C(=O)N2Cc3nc(Cl)nc(C[C@H](C)c4cc(N)cc(C(F)(F)F)c4)c3C2)CCOCC1]

or a pharmaceutically acceptable salt, solvate, isomer, prodrug, or tautomer thereof, and
(b) the RAS inhibitor is Compound RAS-(D), or a pharmaceutically acceptable salt, solvate, isomer, prodrug, or tautomer thereof.

Embodiment 28 is a method of Embodiment 4, wherein:
(a) the SOS1 inhibitor is Compound SOS1-(B), or a pharmaceutically acceptable salt, solvate, isomer, prodrug, or tautomer thereof; and
(b) the RAS inhibitor is Compound RAS-(E), or a pharmaceutically acceptable salt, solvate, isomer, prodrug, or tautomer thereof.

Embodiment 29 is a method of Embodiment 4, wherein:
(a) the SOS1 inhibitor is Compound SOS1-(B), or a pharmaceutically acceptable salt, solvate, isomer, prodrug, or tautomer thereof; and
(b) the RAS inhibitor is Compound RAS-(B), or a pharmaceutically acceptable salt, solvate, isomer, prodrug, or tautomer thereof.

Embodiment 30 is a method of Embodiment 5, wherein:
(a) the SOS1 inhibitor is BI-3406, having the structure:

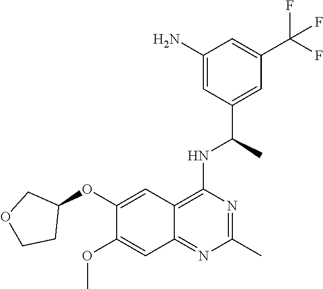 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer, prodrug, or tautomer thereof, and
(b) the RAS inhibitor is Compound RAS-(A), or a pharmaceutically acceptable salt, solvate, isomer, prodrug, or tautomer thereof.

Embodiment 31 is a method of Embodiment 5, wherein:
(a) the SOS1 inhibitor is BI-3406, having the structure:

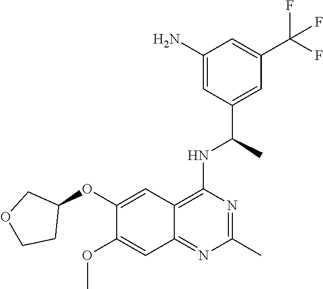 [SMILES: COc1cc2nc(C)nc(N[C@H](C)c3cc(N)cc(C(F)(F)F)c3)c2cc1O[C@H]1CCOC1]

or a pharmaceutically acceptable salt, solvate, isomer, prodrug, or tautomer thereof, and
(b) the RAS inhibitor is Compound RAS-(C), or a pharmaceutically acceptable salt, solvate, isomer, prodrug, or tautomer thereof.

Embodiment 32 is a method of any one of Embodiments 1 through 31, wherein the disease or disorder is selected from the group consisting of tumors of hematopoietic and lymphoid system; a myeloproliferative syndrome; a myelodysplastic syndromes; leukemia; acute myeloid leukemia; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian serous cystadenocarcinoma; paraganglioma; pheochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer (e.g., small and/or large intestinal cancer); thyroid cancer; endometrial cancer; cancer of the biliary tract; soft tissue cancer; ovarian cancer; central nervous system cancer (e.g., primary CNS lymphoma); stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma. In some embodiments, the disease or disorder is selected from brain glioblastoma (GBM), lung adenocarcinoma, colon adenocarcinoma (CRC), bone marrow leukemia, acute myelocytic leukemia (AML), breast carcinoma (NOS), unknown primary melanoma, non-small cell lung carcinoma (NOS), skin melanoma, breast invasive ductal carcinoma (IDC), lung squamous cell carcinoma (SCC), unknown primary adenocarcinoma, bone marrow multiple myeloma, gastroesophageal junction adenocarcinoma, bone marrow myelodysplastic syndrome (MDS), prostate acinar adenocarcinoma, bladder urothelial (transitional cell) carcinoma, uterus endometrial adenocarcinoma (NOS), bone marrow leukemia B cell acute (B-ALL), stomach adenocarcinoma (NOS), and unknown primary carcinoma (NOS).

Embodiment 33 is a method of any one of Embodiments 1 through 32, wherein the RAS inhibitor targets a wild-type RAS protein.

Embodiment 34 is a method of any one of Embodiments 1 through 32, wherein the RAS inhibitor targets a RAS protein mutation.

Embodiment 35 is a method Embodiment 34, wherein the RAS protein mutation is at a position selected from the group consisting of G12, G13, Q61, A146, K117, L19, Q22, V14, A59, and a combination thereof.

Embodiment 36 is a method Embodiment 34, wherein the mutation is selected from the group consisting of G12, G13, and Q61.

Embodiment 37 is a method Embodiment 36, wherein the mutation is selected from the group consisting of G12C, G12D, G12A, G12S, G12V, G13C, G13D, Q61K, and Q61L.

Embodiment 38 is a method of any one of Embodiments 33 through 37, wherein the RAS protein is KRAS.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Cell Lines Used in the Examples

The following table shows the cell lines used in the Examples.

| Cell Line | Species | Histotype | Genotype |
|---|---|---|---|
| NCI-H1573 | Human | NSCLC | KRAS^{G12A} |
| SW1116 | Human | CRC | KRAS^{G12A} |
| MIAPaCa-2 | Human | Pancreatic | KRAS^{G12C} |
| SW1463 | Human | CRC | KRAS^{G12C} |
| SW837 | Human | CRC | KRAS^{G12C} |
| A-427 | Human | NSCLC | KRAS^{G12D} |
| AsPC-1 | Human | Pancreatic | KRAS^{G12D} |
| LS180 | Human | CRC | KRAS^{G12D} |
| LS513 | Human | CRC | KRAS^{G12D} |
| LS123 | Human | CRC | KRAS^{G12S} |
| Capan-1 | Human | Pancreatic | KRAS^{G12V} |
| SW480 | Human | CRC | KRAS^{G12V} |
| NCI-H1355 | Human | NSCLC | KRAS^{G13C} |
| HCT 116 | Human | CRC | KRAS^{G13D} |
| NCI-H1944 | Human | NSCLC | KRAS^{G13D} |
| Calu-6 | Human | NSCLC | KRAS^{Q61K} |
| SW948 | Human | CRC | KRAS^{Q61L} |
| KYSE-410 | Human | Esophageal | KRAS^{G12C} |
| NCI-H358 | Human | Lung | KRAS^{G12C} |
| SNU-C2B | Human | CRC | KRAS^{G12D} |
| HCT-15 | Human | CRC | KRAS^{G13D} |
| TOV-21G | Human | Ovarian | KRAS^{G13C} |

### Methods Used in the Examples

### CrownSyn™ Methods (3-Dimensional Surface Plots)

Cells were grown in 3-dimensional culture in the appropriate growth medium containing 0.65% methylcellulose. On the day of cell seeding, the cells were harvested from 2-dimensional culture during the logarithmic growth period, mixed with appropriate cell media and centrifuged at 1000 rpm for 4 minutes. Cells were re-suspended and counted using CountStar. 3.5 mL of cell suspension was mixed with 6.5 mL of 1% methylcellulose, yielding 10 ml of cell suspension in 0.65% methylcellulose solution. 90 μL cell suspension was added to 96-well plates. Another plate prepared for TO reading. Plates were incubated in humidified incubator at 37° C. with 5% CO₂. Test articles were diluted using DMSO or culture medium to 10× working solution. 10 μl each test article solution was dispensed separately to each well (triplicate for each concentration). Plates were cultured for 120 hr in humidified incubator at 37° C. with 5% CO2 or 100% air. For TO reading, 10 μl culture medium added to each well of TO plate, and cell viability determined using CTG assay as described below. After 120 hours, plates were equilibrated at room temperature for approximately 30 minutes, prior to addition of 100 μl of CellTiter-Glo® Reagent into each assay well. Contents mixed for 2 minutes on an orbital shaker to induce cell lysis. Plates were allowed to incubate at room temperature for 10 minutes to stabilize luminescent signal. Luminescence was recorded using EnVision MultiLabel Reader. The software of GraphPad Prism used to calculate IC50. The graphical curves were fitted using a nonlinear regression model with a sigmoidal dose response. Two dimensional concentration-response surfaces were compared to two different additivity models, Bliss independence and Loewe additivity. Deviation from the prediction of either model was assessed in the form of a synergy score. Synergy scores greater than 5 with either model and at any point on the concentration response surface were interpreted as indicating a significant interaction between compounds. See Bliss C.I. (1939) The toxicity of poisons applied jointly. Ann. Appl. Biol., 26, 585-615, and see Loewe S. (1953) The problem of synergism and antagonism of combined drugs. ArzneimiettelForschung, 3,2 86-290. The Bliss independence model is expected to hold true for non-interacting drugs that elicit their responses independently, e.g., by targeting separate pathways. Loewe additivity, in contrast, is more compatible with the cases where both drugs have similar modes of action on the same targets or pathways. As pointed out in the Saariselka agreement (Greco et al., 1992), and also by many others, neither Loewe additivity nor Bliss independence is necessarily reflecting the expected modes of action of a drug combination. Rather, Loewe and Bliss models should be used as data exploratory approaches, with a major purpose to identify potential synergistic drug combinations that warrant further mechanistic investigation, but not the other way around, i.e., using the mechanistic evidence to determine which reference model is more appropriate.

### 2-Dimensional Potency Shifts

Cells were grown in 2-dimensional culture. Assay stocks were thawed and diluted in the recommended ATCC medium, supplemented with 10% serum and 1% pen/strep (final concentration) and dispensed in a 384-well plate. Depending on the cell line used, a cell density of 100-6400 cells per well in 45 μl medium was used. The margins of the plate were filled with phosphate-buffered saline. Plated cells were incubated in a humidified atmosphere of 5% CO2 at 37° C. After 24 hours, 5 μl of reference compound dilution, containing the single dose compound at indicated concentration was added to the plates and these were further incubated for 120 hrs. At t=120 hours, 24 μl of ATPlite 1Step™ (PerkinElmer) solution was added to each well, and subsequently shaken for 2 minutes. After 5 minutes incubation in the dark, the luminescence was recorded on an Envision multilabel reader (PerkinElmer). On a parallel t=0 plate, 45 μl cells were dispensed and incubated in a humidified atmosphere of 5% CO2 at 37° C. After 24 hours 5 μl DMSO-containing Hepes buffer and 24 μl ATPlite 1Step™ solution were mixed, and luminescence measured after 5 minutes incubation (=luminescenceₜ₌₀). Curves and IC₅₀s were calculated by non-linear regression using IDBS XLfit 5. The percentage growth after incubation until t=end (% growth) was calculated as follows: 100%×(luminescence _{t=end}/luminescence_{untreated,t=end}). This was fitted to the ¹⁰ log compound concentration (conc) by a 4 parameter sigmoidal curve: %-growth bottom+(top−bottom) (1+10(logIC₅₀-conc)*hill)) where hill is the Hill-coefficient, and bottom and top the asymptotic minimum and maximum cell growth that the compound allows in that assay.

### Inhibition of Phospho-ERK

Phospho ERK in cells was determined using the MSD® platform. NCI-H1355 or TOV-21G cells were plated in clear flat-bottom 96-well tissue culture plates at 30,000 cells/well in 100 μL/well in complete media, and placed in incubator (37° C., 5% COB) overnight. All compounds were reconstituted in DMSO to reach 1000× of desired top concentration and arrayed in column 1 of a 96-well plate. 3-fold serial dilution performed in DMSO of all compounds across the 96-well plate, leaving column 11 with 100% DMSO and column 12 empty. Compounds were diluted in media 1:500 and mixed well. Dilution series at 2× final concentration were then mixed with 2× single dose compounds or DMSO vehicle. Cell media was aspirated from cell plates, and 100 μL of 1× compound mixture was added. Plates were placed in incubator (37° C., 5% CO2) for 4 hours. MSD® lysis buffer was prepared immediately before time point by mixing 10 mL Tris Lysis Buffer (provided in MSD® kit), 1 tablet PhosSTOP EASYpack (Roche), 1 tablet cOmplete Mini, EDTA-free Protease Inhibitor Cocktail (Roche), 40 μL PMSF (provided in MSD® kit), 100 μL SDS (provided in MSD® kit), and kept on ice before use. After treatment time, media was aspirated from plates and 50 μL MSD® lysis buffer added to each well. Plates were sealed with foil adhesive and shaken for 5 minutes at 750 rpm at room temperature. Plates were then incubated on ice for 15 minutes, and stored at −80° C. MSD® Phospho(Thr202/Tyr204; Thr185/Tyr187)/Total ERK1/2 Assay performed according to manufacturer protocol.

### Example 1. CrownBio Data Obtained from Combination of SOS1 Inhibitor and RAS Inhibitor on a Variety of Cell Lines

Data were obtained according to the CrownSyn™ method on a variety of cell lines treated with a combination of a SOS1 inhibitor and a RAS inhibitor.

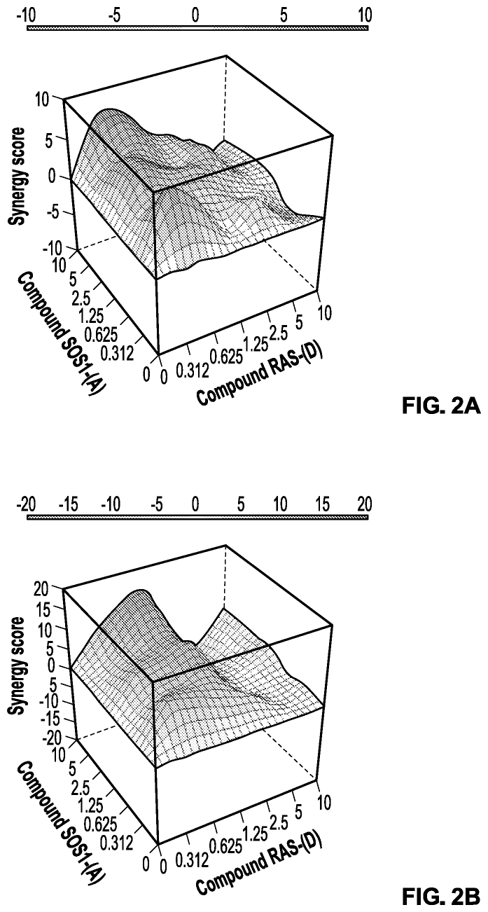

FIGS. 2A (Bliss Score) and 2B (Loewe Score) are three-dimensional surface plots depicting the in vitro combination effect of Compound SOS1-(A) and Compound RAS-(D) observed in NCI-H1355 cells. A synergy score >5 at any point on the plot indicates a positive interaction between the two compounds.

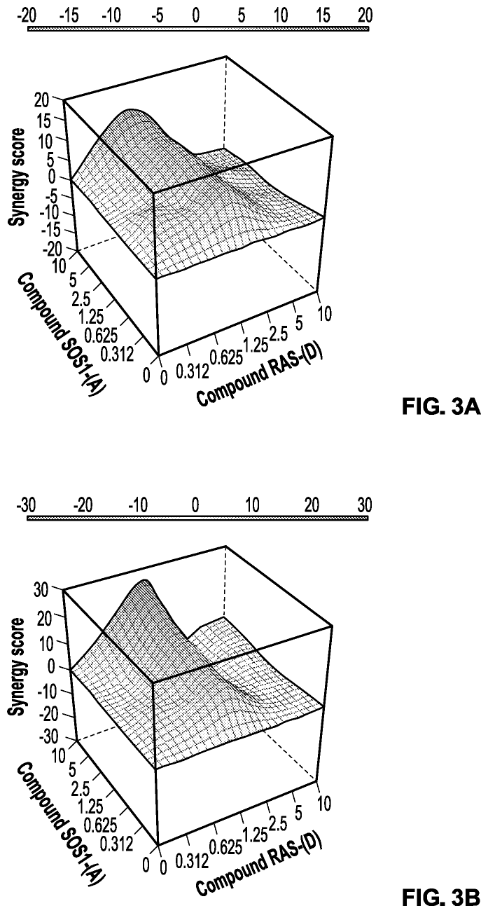

FIG. 3A (Bliss Score) and 3B (Loewe Score) are three-dimensional surface plots depicting the in vitro combination effect of Compound SOS1-(A) and Compound RAS-(D) observed in NCI-H1944 cells. A synergy score >5 at any point on the plot indicates a positive interaction between the two compounds.

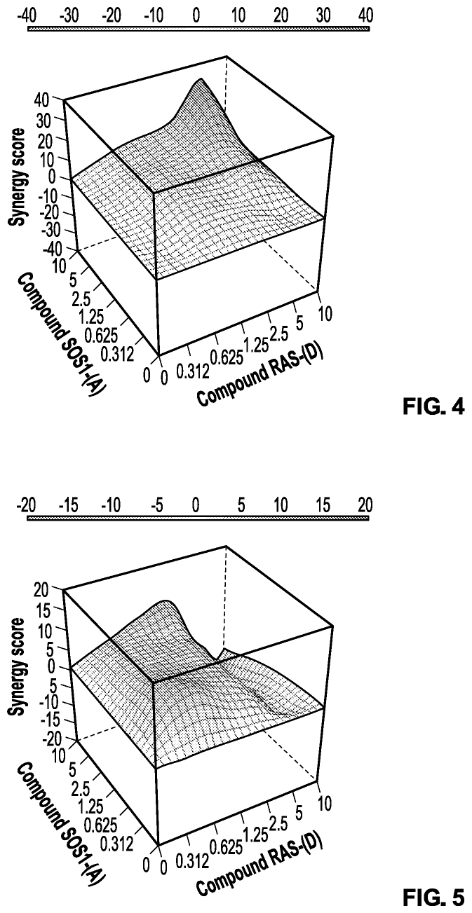

FIG. 4 (Bliss score) is a three-dimensional plot depicting the in vitro combination effect of Compound SOS1-(A) and Compound RAS-(D) observed in SW948 cells. A synergy score >5 at any point on the plot indicates a positive interaction between the two compounds.

FIG. 5 (Loewe Score) is a three-dimensional plot depicting the in vitro combination effect of Compound SOS1-(A) and Compound RAS-(D) observed in A-427 cells. A synergy score >5 at any point on the plot indicates a positive interaction between the two compounds.

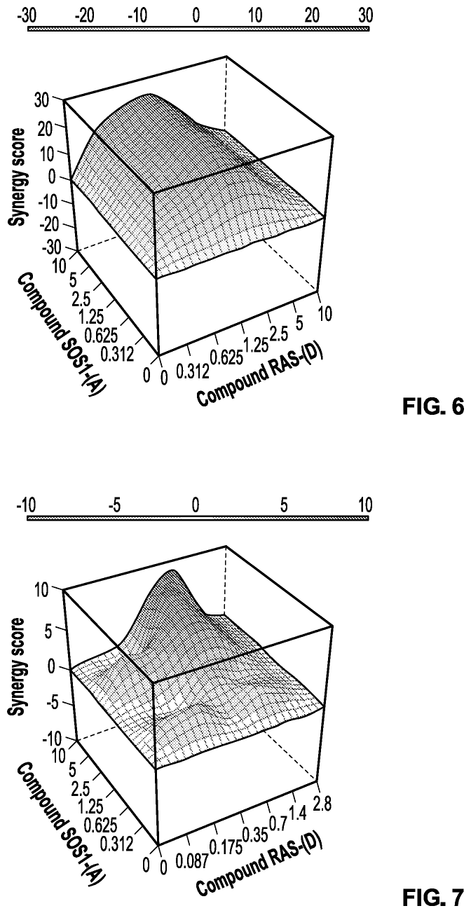

FIG. 6 (Bliss Score) is a three-dimensional plot depicting the in vitro combination effect of Compound SOS1-(A) and Compound RAS-(D) observed in LS 180 cells. A synergy score >5 at any point on the plot indicates a positive interaction between the two compounds.

FIG. 7 (Bliss Score) is a three-dimensional plot depicting the in vitro combination effect of Compound SOS1-(A) and Compound RAS-(D) observed in AsPC-1 cells. A synergy score >5 at any point on the plot indicates a positive interaction between the two compounds.

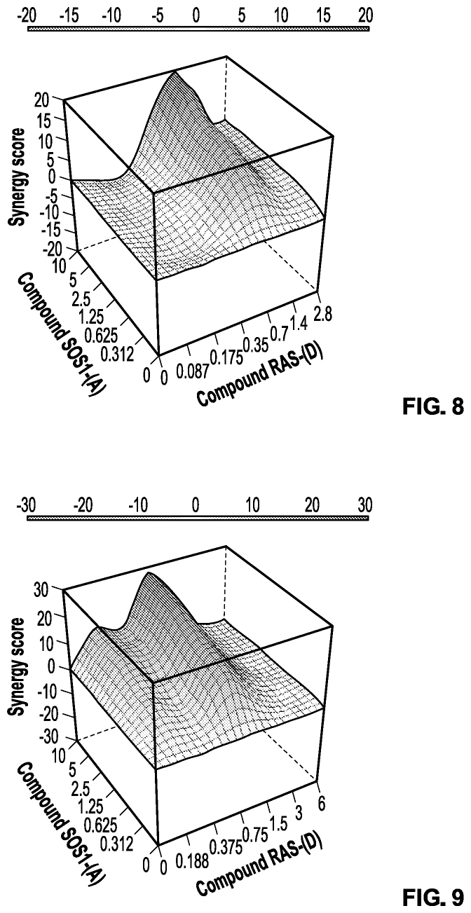

FIG. 8 (Bliss Score) is a three-dimensional plot depicting the in vitro combination effect of Compound SOS1-(A) and Compound RAS-(D) observed in HCT 116 cells. A synergy score >5 at any point on the plot indicates a positive interaction between the two compounds.

FIG. 9 (Bliss Score) is a three-dimensional plot depicting the in vitro combination effect of Compound SOS1-(A) and Compound RAS-(D) observed in SW480 cells. A synergy score >5 at any point on the plot indicates a positive interaction between the two compounds.

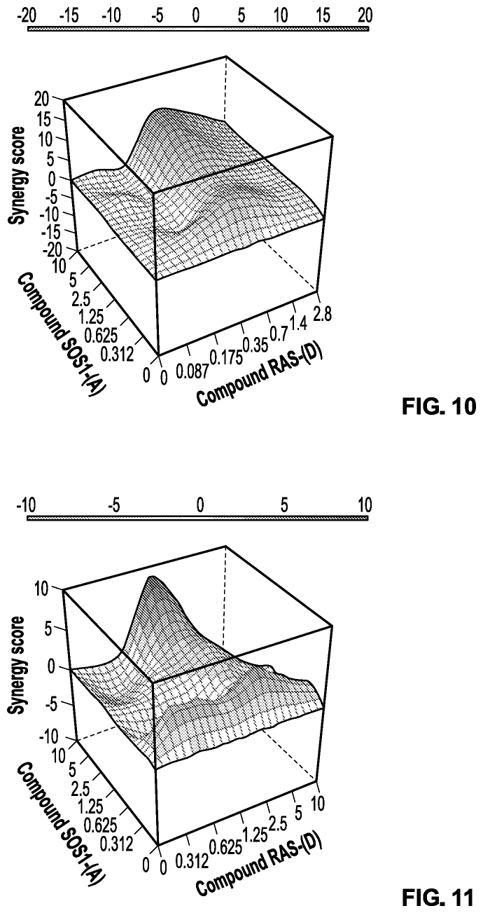

FIG. 10 (Bliss Score) is a three-dimensional plot depicting the in vitro combination effect of Compound SOS1-(A) and Compound RAS-(D) observed in Calu-6 cells. A synergy score >5 at any point on the plot indicates a positive interaction between the two compounds.

FIG. 11 (Bliss Score) is a three-dimensional plot depicting the in vitro combination effect of Compound SOS1-(A) and Compound RAS-(D) observed in NCI-H1573 cells. A synergy score >5 at any point on the plot indicates a positive interaction between the two compounds.

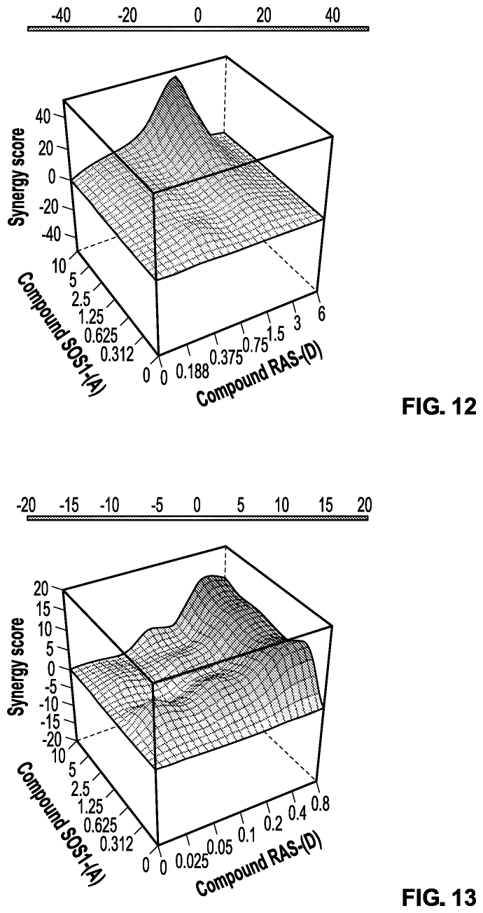

FIG. 12 (Bliss Score) is a three-dimensional plot depicting the in vitro combination effect of Compound SOS1-(A) and Compound RAS-(D) observed in LS513 cells. A synergy score >5 at any point on the plot indicates a positive interaction between the two compounds.

FIG. 13 (Bliss Score) is a three-dimensional plot depicting the in vitro combination effect of Compound SOS1-(A) and Compound RAS-(D) observed in Capan-1 cells. A synergy score >5 at any point on the plot indicates a positive interaction between the two compounds.

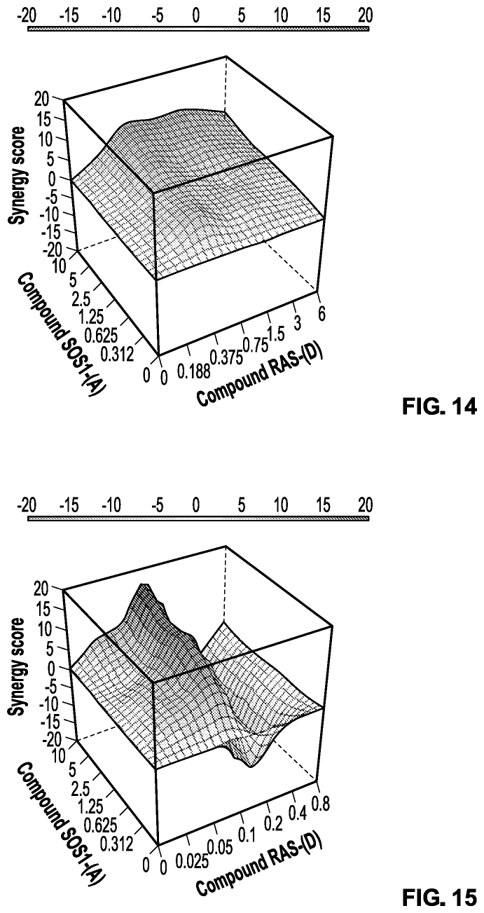

FIG. 14 (Bliss Score) is a three-dimensional plot depicting the in vitro combination effect of Compound SOS1-(A) and Compound RAS-(D) observed in SW837 cells. A synergy score >5 at any point on the plot indicates a positive interaction between the two compounds.

FIG. 15 (Loewe Score) is a three-dimensional plot depicting the in vitro combination effect of Compound SOS1-(A) and Compound RAS-(D) observed in MIA PaCa-2 cells. A synergy score >5 at any point on the plot indicates a positive interaction between the two compounds.

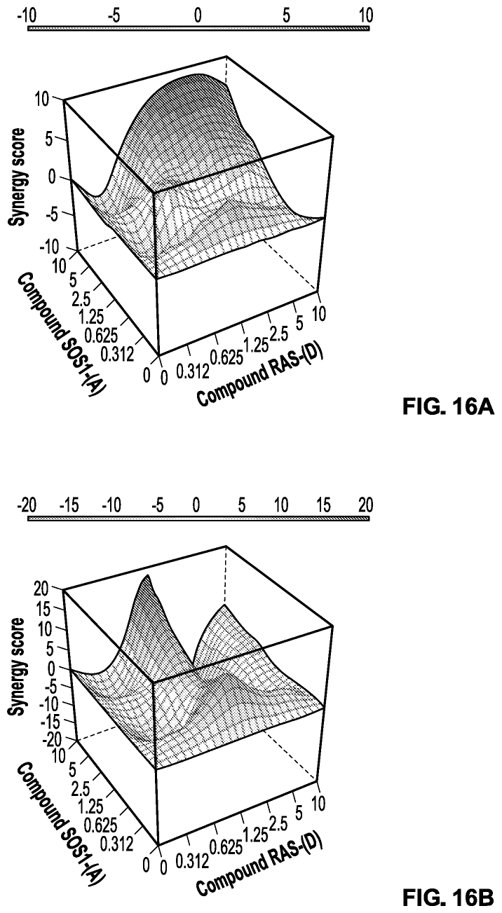

FIG. 16A (Bliss Score) and 16B (Loewe Score) are three-dimensional plots depicting the in vitro combination effect of Compound SOS1-(A) and Compound RAS-(D) observed in SW1116 cells. A synergy score >5 at any point on the plot indicates a positive interaction between the two compounds.

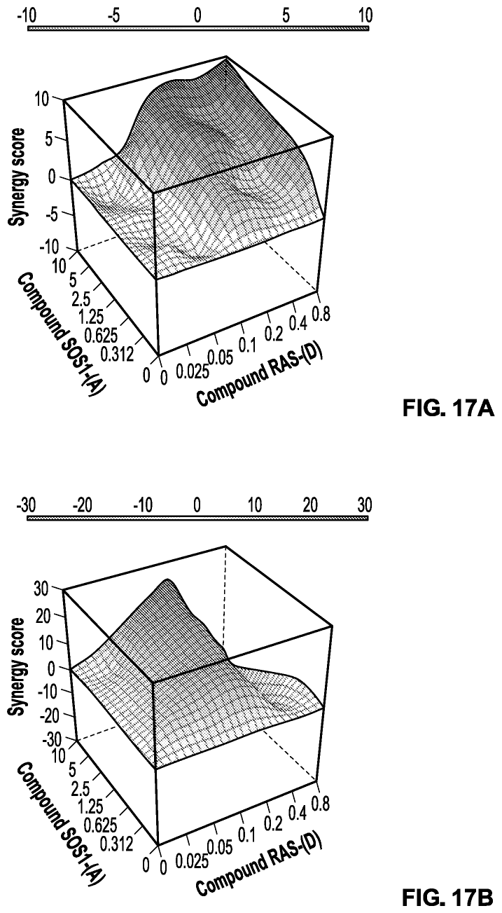

FIG. 17A (Bliss Score) and 17B (Loewe Score) are three-dimensional plots depicting the in vitro combination effect of Compound SOS1-(A) and Compound RAS-(D) observed in LS123 cells. A synergy score >5 at any point on the plot indicates a positive interaction between the two compounds.

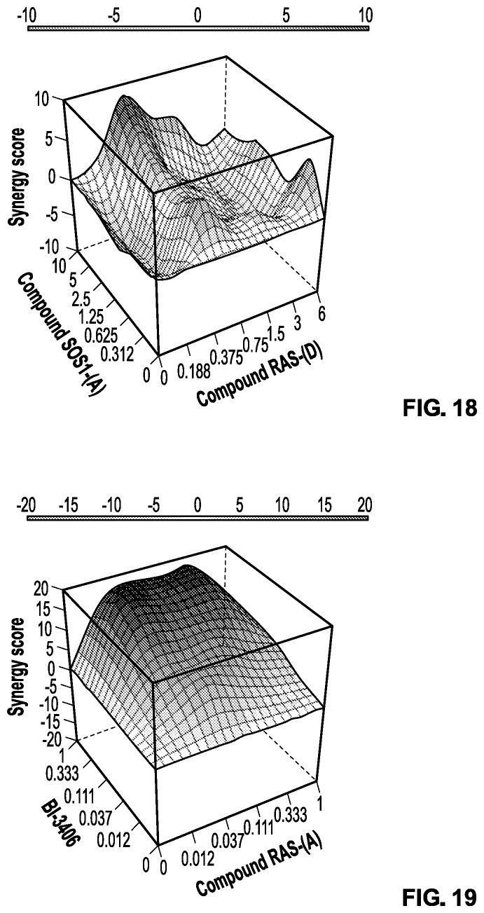

FIG. 18 (Bliss Score) is a three-dimensional plot depicting the in vitro combination effect of Compound SOS1-(A) and Compound RAS-(D) observed in SW1463 cells. A synergy score >5 at any point on the plot indicates a positive interaction between the two compounds.

### Example 2. CrownBio Data Obtained from Combination of SOS1 Inhibitor and RAS Inhibitor on a Variety of Cell Lines

Data were obtained according to the CrownSyn™ method on a variety of cell lines treated with a combination of a SOS1 inhibitor and a RAS inhibitor.

FIG. 19 (Loewe Score) is a three-dimensional plot depicting the in vitro combination effect of BI-3406 (SOS1 inhibitor) and Compound RAS-(A) observed in KYSE-410 cells. A synergy score >5 at any point on the plot indicates a positive interaction between the two compounds.

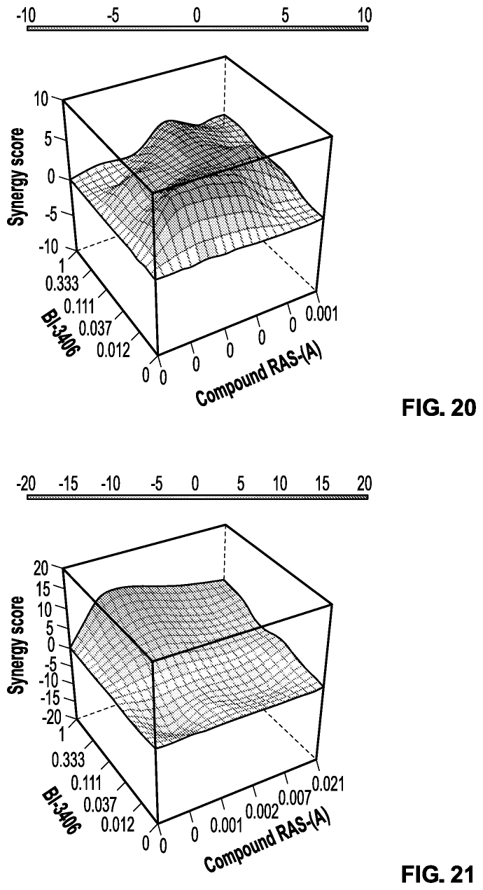

FIG. 20 (Bliss Score) is a three-dimensional plot depicting the in vitro combination effect of BI-3406 (SOS1 inhibitor) and Compound RAS-(A) observed in NCI-H358 cells. A synergy score >5 at any point on the plot indicates a positive interaction between the two compounds.

FIG. 21 (Loewe Score) is a three-dimensional plot depicting the in vitro combination effect of BI-3406 (SOS1 inhibitor) and Compound RAS-(A) observed in SW837 cells. A synergy score >5 at any point on the plot indicates a positive interaction between the two compounds.

### Example 3. SOS1 Inhibitor Shows Combination Benefits with RAS(ON) Inhibitors in Vitro

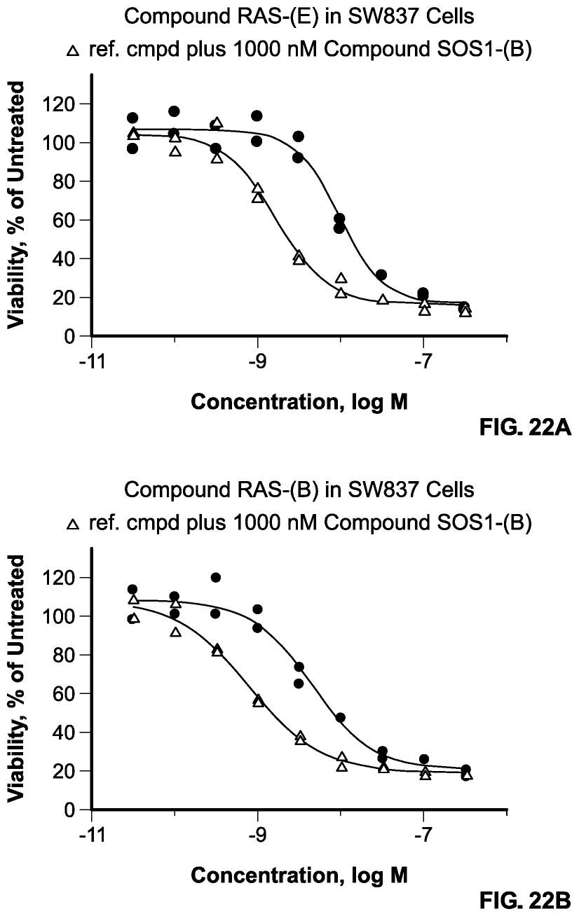

In this example, SW837 cells (colorectal cancer, human) were characterized. The SW837 cell line contained the KRAS^{G12C} mutation. The cell lines were treated with DMSO (vehicle) and a constant concentration (1000 nM, 1 μM) of SOS1 inhibitor, Compound SOS1-(B). The cell lines were treated with varying concentrations of RAS inhibitor, Compound RAS-(E) and Compound RAS-(B). FIGS. 22A and 22B are graphs depicting the in vitro combination effect of SOS1 inhibitor Compound SOS1-(B) and RAS inhibitor Compound RAS-(E) (FIG. 22A) or Compound RAS-(B) (FIG. 22B) observed in SW837 cells. Effect of RAS(ON) inhibitor as a single agent is shown with black circles. Effect of RAS(ON) inhibitor in combination with 1 μM SOS1 inhibitor is shown with grey triangles. An increase in apparent potency (left-ward shift of curve) in the combination is an indication of a positive interaction between the compounds. The data were obtained according to the 2-dimensional potency shifts experimental protocol.

### Example 4. SOS1 Inhibitor Shows Combination Benefits with RAS(ON) Inhibitors in Vitro

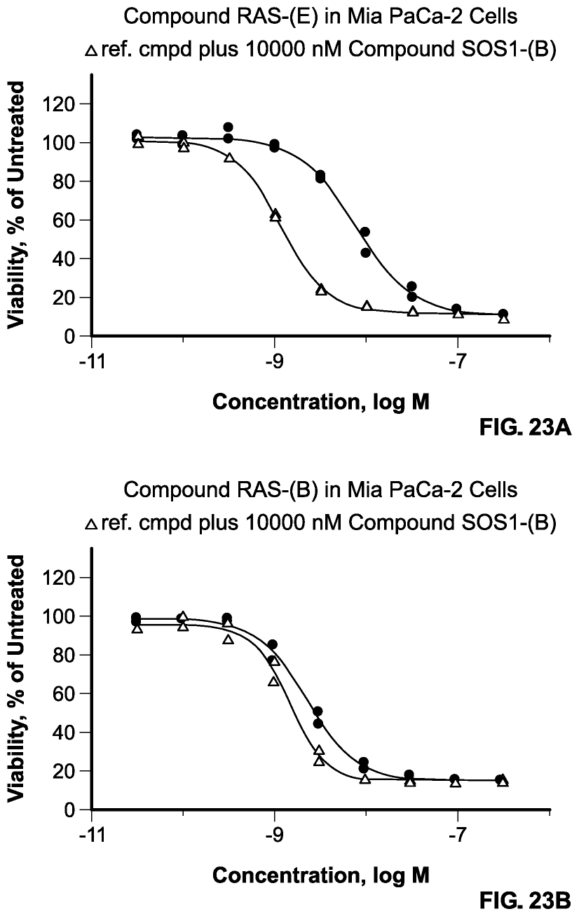

In this example, MIA PaCa-2 cells (pancreatic cancer, human) were characterized. The MIA PaCa-2 cell line contained the KRAS^{G12C} mutation. The cell lines were treated with DMSO (vehicle) and a constant concentration (10,000 nM, 10 μM) of SOS1 inhibitor, Compound SOS1-(B). The cell lines were treated with varying concentrations of RAS inhibitor, Compound RAS-(E) and Compound RAS-(B). FIGS. 23A and 23B are graphs depicting the in vitro combination effect of SOS1 inhibitor Compound SOS1-(B) and RAS inhibitor Compound RAS-(E) (FIG. 23A) or Compound RAS-(B) (FIG. 23B) observed in MIA PaCa-2 cells. Effect of RAS(ON) inhibitor as a single agent is shown with black circles. Effect of RAS(ON) inhibitor in combination with 1 μM SOS1 inhibitor is shown with grey triangles. An increase in apparent potency (left-ward shift of curve) in the combination is an indication of a positive interaction between the compounds. The data were obtained according to the 2-dimensional potency shifts experimental protocol.

### Example 5. SOS1 Inhibitor Shows Combination Benefits with RAS(ON) Inhibitors in Vitro

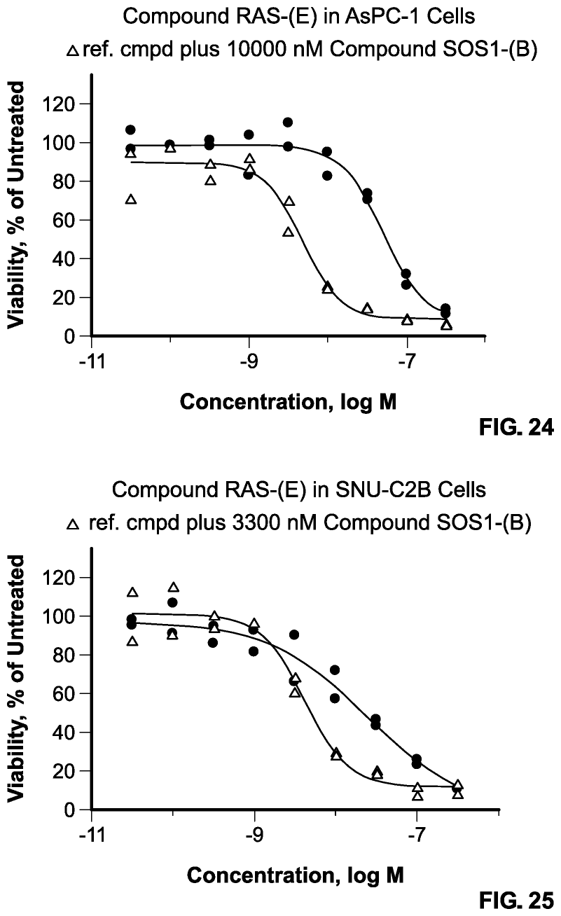

In this example, AsPC-1 cells (pancreatic cancer, human) were characterized. The AsPC-1 cell line contained the KRAS^{G12D} mutation. The cell lines were treated with DMSO (vehicle) and a constant concentration (10,000 nM, 10 μM) of SOS1 inhibitor, Compound SOS1-(B). The cell lines were treated with varying concentrations of RAS inhibitor Compound RAS-(E). FIG. 24 is a graph depicting the in vitro combination effect of SOS1 inhibitor Compound SOS1-(B) and RAS inhibitor Compound RAS-(E) observed in AsPC-1 cells. Effect of RAS(ON) inhibitor as a single agent is shown with black circles. Effect of RAS(ON) inhibitor in combination with 1 μM SOS1 inhibitor is shown with grey triangles. An increase in apparent potency (left-ward shift of curve) in the combination is an indication of a positive interaction between the compounds. The data were obtained according to the 2-dimensional potency shifts experimental protocol.

### Example 6. SOS1 Inhibitor Shows Combination Benefits with RAS(ON) Inhibitors in Vitro

In this example, SNU-C2B cells (colorectal cancer, human) were characterized. The SNU—C2B cell line contained the KRAS^{G12D} mutation. The cell lines were treated with DMSO (vehicle) and a constant concentration (3300 nM, 3.3 μM) of SOS1 inhibitor, Compound SOS1-(B). The cell lines were treated with varying concentrations of RAS inhibitor Compound RAS-(E). FIG. 25 is a graph depicting the in vitro combination effect of SOS1 inhibitor Compound SOS1-(B) and RAS inhibitor Compound RAS-(E) observed in SNU—C2B cells. Effect of RAS(ON) inhibitor as a single agent is shown with black circles. Effect of RAS(ON) inhibitor in combination with 1 μM SOS1 inhibitor is shown with grey triangles. An increase in apparent potency (left-ward shift of curve) in the combination is an indication of a positive interaction between the compounds. The data were obtained according to the 2-dimensional potency shifts experimental protocol.

### Example 7. SOS1 Inhibitor Shows Combination Benefits with RAS(ON) Inhibitors in Vitro

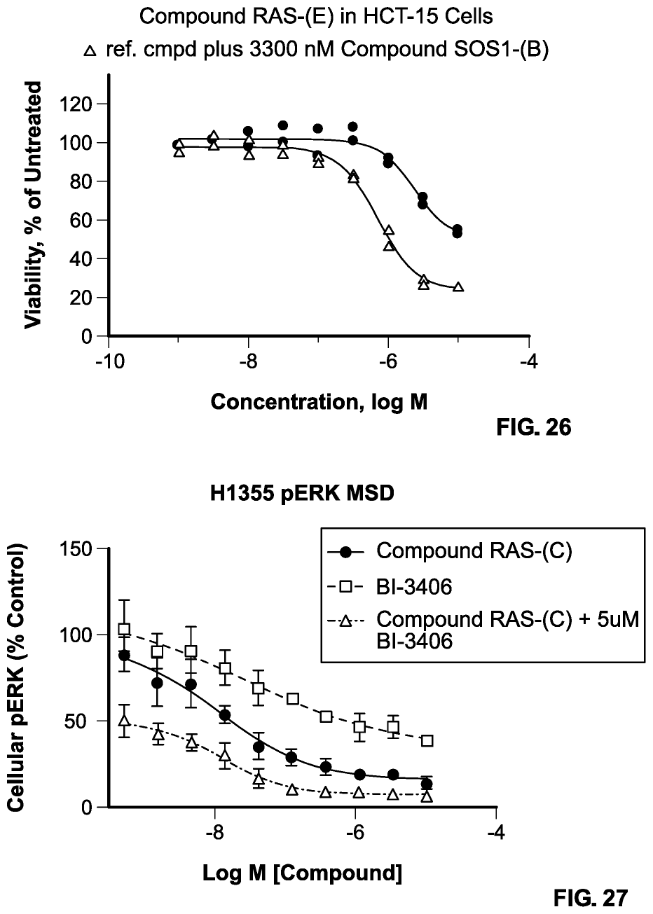

In this example, HCT-15 cells (colorectal cancer, human) were characterized. The HCT-15 cell line contained the KRAS^{G13D} mutation. The cell lines were treated with DMSO (vehicle) and a constant concentration (3300 nM, 3.3 μM) of SOS1 inhibitor, Compound SOS1-(B). The cell lines were treated with varying concentrations of RAS inhibitor, Compound RAS-(E) and Compound RAS-(B). FIG. 26 is a graph depicting the in vitro combination effect of SOS1 inhibitor Compound SOS1-(B) and RAS inhibitor Compound RAS-(E) observed in HCT-15 cells. Effect of RAS(ON) inhibitor as a single agent is shown with black circles. Effect of RAS(ON) inhibitor in combination with 1 μM SOS1 inhibitor is shown with grey triangles. An increase in apparent potency (left-ward shift of curve) in the combination is an indication of a positive interaction between the compounds. The data were obtained according to the 2-dimensional potency shifts experimental protocol.

### Example 8. SOS1 Inhibitor Shows Combination Benefits with RAS(ON) Inhibitors in Vitro

In this example, H1355 cells (NSCLC, human) were characterized. The HCT-15 cell line contained the KRAS^{G13C} mutation. The phospho-ERK protocol was used. The cell lines were treated with a constant concentration (5 μM) of SOS1 inhibitor, BI-3406. The cell lines were treated with varying concentrations of RAS^{G13C}(ON) inhibitor Compound RAS-(C). FIG. 27 is a graph showing the in vitro combination effect of SOS1 inhibitor BI-3406 and RAS^{G13C}(ON) inhibitor Compound RAS-(C) observed in NCI-H1355 cells. Effect of RAS(ON) inhibitor as a single agent is shown with black circles. Effect of RAS(ON) inhibitor in combination with 5 μM SOS1 inhibitor is shown with grey triangles. An increase in the depth of maximum response in the combination is an indication of a positive interaction between the compounds.

### Example 9. SOS1 Inhibitor Shows Combination Benefits with RAS(ON) Inhibitors in Vitro

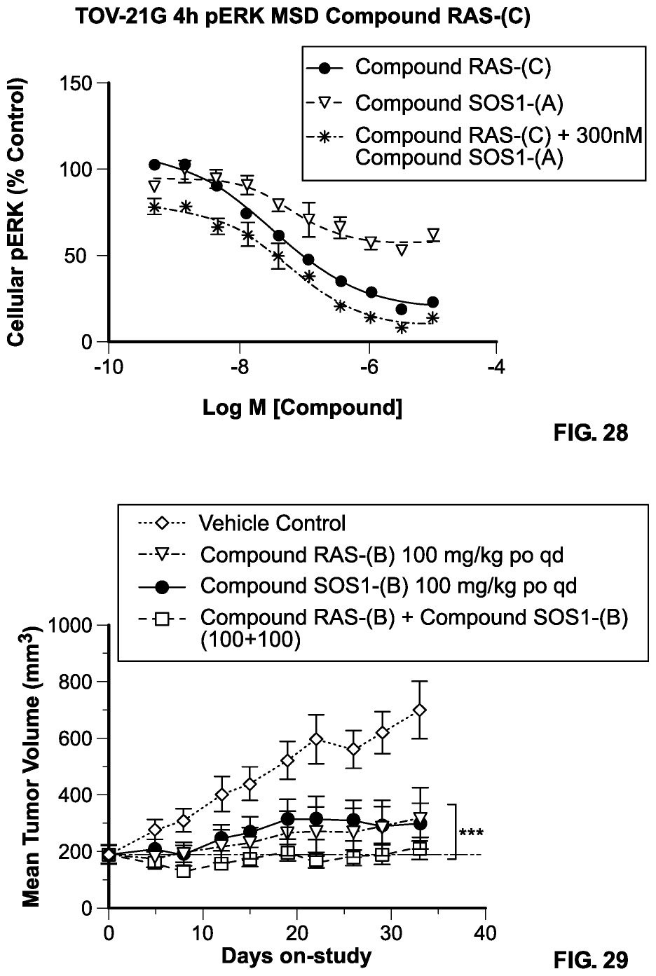

In this example, TOV-21G cells (ovarian cancer, human) were characterized. The HCT-15 cell line contained the KRAS^{G13C} mutation. The phospho-ERK protocol was used. The cell lines were treated with a constant concentration (300 nM) of SOS1 inhibitor, Compound SOS1-(A). The cell lines were treated with varying concentrations of RAS^{G13C}(ON) inhibitor Compound RAS-(C). FIG. 28 is a graph showing the in vitro combination effect of SOS1 inhibitor Compound SOS1-(A) and RAS^{G13C}(ON) inhibitor Compound RAS-(C) observed in TOV-21G cells. Effect of RAS^{G13C}(ON) inhibitor as a single agent is shown with black circles. Effect of RAS(ON) inhibitor in combination with 300 nM SOS1 inhibitor is shown with grey circles. An increase in the depth of maximum response in the combination is an indication of a positive interaction between the compounds.

### Example 10. Effect of SOS1 Inhibitor (Compound SOS1-(B)) on In Vivo Tumor Cell Growth Alone and in Combination with KRAS^{G12C} Inhibitor (Compound RAS-(B))

Objective: To evaluate the efficacy of the SOS1 inhibitor Compound SOS1-(B) alone and in combination with a KRAS G12C inhibitor Compound RAS-(B) following oral administration in the human esophageal squamous cell carcinoma, KYSE-410 xenograft model in nude mice

Methods: The effect of a SOS1 inhibitor on tumor cell growth in vivo was evaluated in the KYSE-410 xenograft model using female balb/c athymic nude mice (6-8 weeks old). Mice were implanted with KYSE-410 tumor cells in 50% matrigel (5^{e6}cells/mouse) subcutaneously in the flank. Once tumors reached an average size of ˜200 mm³ mice were randomized to treatment groups and administration of test article or vehicle (25% DMSO/50% PEG 400/25% Solutol/+60% 2% HMPC in 50 mM Sodium Citrate pH 4). Body weight and tumor volume (using digital calipers) were measured twice a week until study endpoints. Compounds were administered by oral gavage daily.

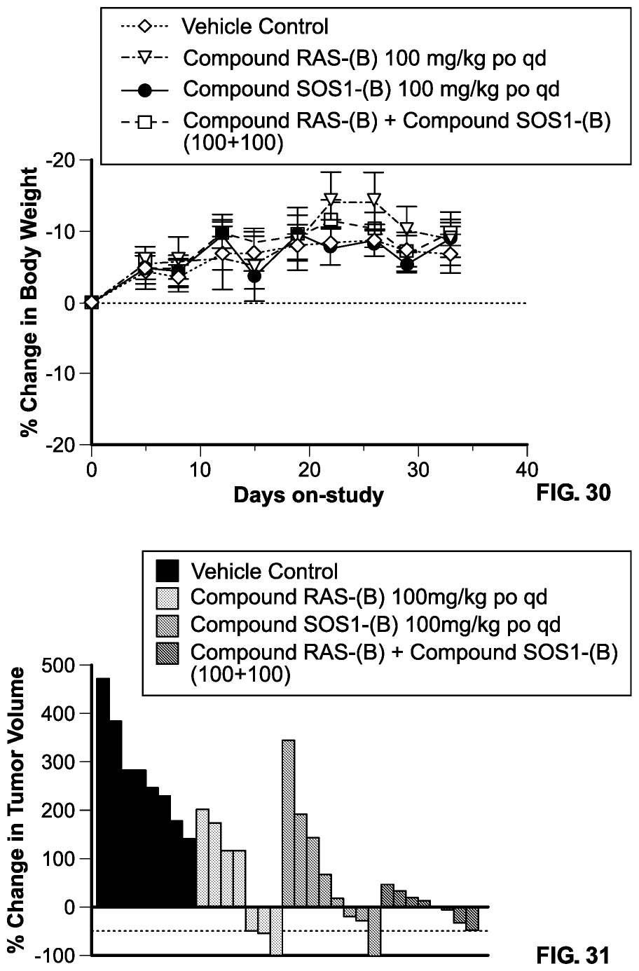

Results: FIG. 29 shows the efficacy of repeated daily dosing of Compound SOS1-(B) at 100 mg/kg po (tumor growth inhibition, TGI 79%) and Compound RAS-(B) at 100 mg/kg (75%). See also FIG. 31, depicting the % change in tumor volume. Compound SOS1-(B) at 100 mg/kg and Compound RAS-(B) as a single agent caused significant tumor growth inhibition compared to the vehicle control, ***p≤0.001 as assessed by an ordinary one-way ANOVA of tumor volumes along with multiple comparisons via a post-hoc Tukey's test in Graphpad Prism Software.

When dosed in combination, Compound SOS1-(B) at 100 mg/kg with Compound RAS-(B) at 100 mg/kg produces average regressions of 96%. At the end of study, 3/7 mice in the Compound RAS-(B), 3/8 in the Compound SOS1-(B), and 2/8 mice in the combination groups achieved tumor regressions >10% reduction from baseline.

As shown in FIG. 30, all treatments were well-tolerated for the duration of the study as evaluated by body weight

Conclusion: Compound SOS1-(B) exhibits statistically significant efficacy in the KYSE-410 human esophageal squamous cell carcinoma model following oral administration at 100 mg/kg daily. Compound RAS-(B) also exhibited efficacy in this model at a 100 mg/kg daily dose. Compound SOS1-(B) as a single agent and in combination with Compound RAS-(B) was well tolerated and the combination regimen resulted in 2/8 tumor regressions at the end of study and overall 96% TGI.

### Example 11. Effect of SOS1 Inhibitor (Compound SOS1-(C)) on In Vivo Tumor Cell Growth Alone and in Combination with KRAS^{G12C} Inhibitor (Compound RAS-(F))

Methods: The combinatorial effects of SOS1-(C) and RAS-(F) on tumor growth in vivo were evaluated in the KRAS^{G12C} human colorectal cancer (CRC) patient-derived xenograft (PDX) model CRC022 using female Balb/c nude mice (6-8 weeks old). Tumor fragments about 15-30 mm³ in size were subcutaneously implanted into the right flank of the mice. Once tumors reached an average size of 150-250 mm³, mice were randomized to treatment groups to start the administration of test articles or vehicle. Both SOS1-(C) and RAS-(F) were administered by oral gavage (PO) daily. Tumor volume (using calipers) was measured twice weekly until study endpoints. Repeated measure 2-way ANOVA multiple comparisons were used to for statistical analysis.

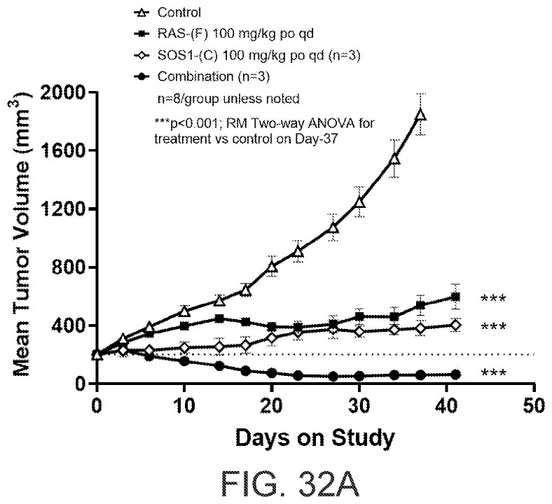

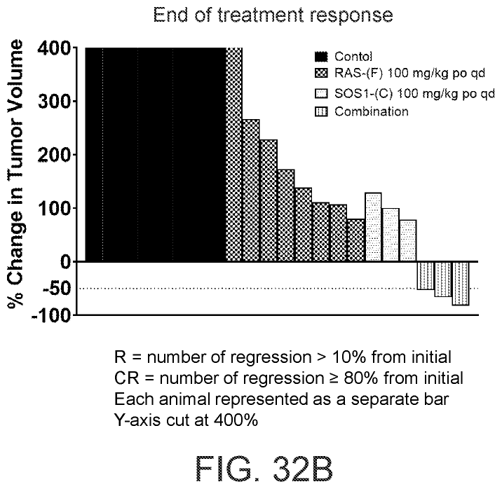

Results: In the human CRC KRAS^{G12C} PDX model CRC022, the combination of SOS1-(C) dosed at 100 mg/kg PO daily plus RAS-(F) 100 mg/kg PO daily resulted in tumor regression, whereas each respective single agent treatment led to tumor growth inhibition (TGI). See FIG. 32A, which shows the efficacy of repeated daily dosing of Compound SOS1-(C) at 100 mg/kg po, Compound RAS-(F) at 100 mg/kg po, or a combination of Compound SOS1-(C) and Compound RAS-(F). The combination treatment caused a mean tumor regression of 68.6% on Day-41 with all 3 tumors regressed more than 10% from initial volume and 1 tumor reached more than 80% regression. The single agent treatment of SOS1-(C) and RAS-(F) led to a mean TGI of 89.1% and 79.6%, respectively, on Day-37 which was the final day for the control group. The plot of mean tumor volumes over the study course is shown in FIG. 32A, and waterfall plot depicting percentage change in tumor volume for individual mouse is shown in FIG. 32B. The differences in mean tumor volumes on Day-37 between all treatment groups versus control are statistically significant.

### Example 12. Effect of SOS1 Inhibitor (Compound SOS1-(C)) on In Vivo Tumor Cell Growth Alone and in Combination with KRAS^{G12C} Inhibitor (Compound RAS-(F))

Methods: The combinatorial effects of SOS1-(C) and RAS-(F) on tumor cell growth in vivo were evaluated in the human non-small cell lung cancer NCI-H2030 (KRAS^{G12C}; STK11^{MUT}; KEAP1^{MUT}) xenograft model using female NOD SCID mice (4-5 weeks old). Mice were implanted with NCI-H2030 tumor cells in 50% Matrigel (1×10⁷ cells/mouse) subcutaneously in the flank. Once tumors reached an average size of 150-200 mm³, mice were randomized to treatment groups to start the administration of test articles or vehicle. Both SOS1-(C) and RAS-(F) were administered by oral gavage daily. Tumor volume (using calipers) was measured twice weekly until study endpoints. Repeated measure 2-way ANOVA multiple comparisons were used to for statistical analysis.

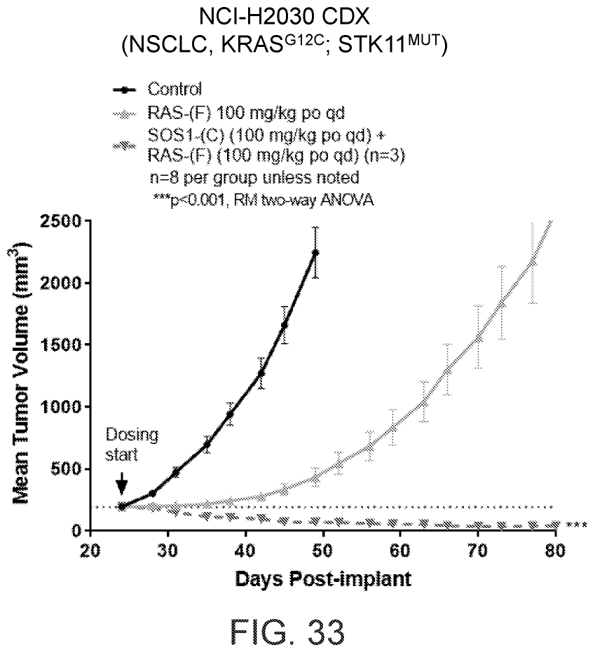

Results: In the human non-small cell lung cancer NCI-H2030 tumors, the combination of SOS1-(C) dosed at 100 mg/kg PO daily plus RAS-(F) 100 mg/kg PO daily resulted in durable tumor regression until the last day of dosing, whereas single agent RAS-(F) caused 88.4% TGI on day 25 post treatment starts as shown by the mean tumor volume plot of FIG. 33. On the last day of dosing (day 56 post treatment starts), combination of SOS1-(C) and RAS-(F) displayed significantly better tumor growth inhibitory effects when compared to RAS-(F) single agent.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

We claim:

1. A method of treating a subject having a RAS protein-related disease or disorder, the method comprising administering to a subject in need of such treatment:
(a) a therapeutically effective amount of a SOS1 inhibitor having the structure of Formula (48-I), 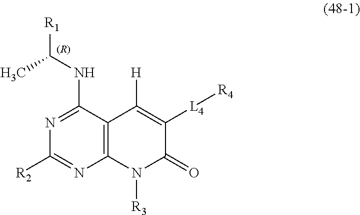
or a pharmaceutically acceptable salt, solvate, isomer, prodrug, or tautomer thereof, wherein:
R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl;
R₂ is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocyclyl, and further wherein R₂ₐ is selected from the group consisting of H, C₁₋₆ alkyl, 3-6 membered heterocyclyl, and C₁₋₆ haloalkyl;
R₃ is selected from the group consisting of H, C₁₋₃ alkyl, —OR₃ₐ, cyclopropyl, and 3-6 membered heterocyclyl, wherein each of C₁₋₃ alkyl, cyclopropyl, and 3-6 membered heterocyclyl is optionally substituted with R₃ₐ, and further wherein R₃a is selected from the group consisting of C₁₋₃ alkyl, halogen, —OH, and —CN;
L₄ is selected from the group consisting of bond, —C(O)—, —C(O)O—, —C(O)NH(CH₂)ₒ—, —NH—, —S—, —S(O)₂—, 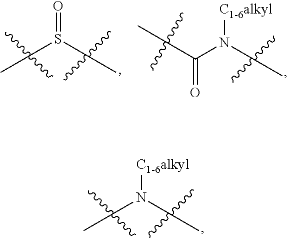
—(CH₂)ₚ—, and —O—; wherein o is 0, 1, or 2; and wherein p is a number from 1 to 6; and
R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, —NR_{4b}R_{4c}, —SO₂R₄ₐ, 3-6 membered cycloalkyl, 3-7 membered heterocyclyl, 6-10 membered aryl, or 5-10 membered heteroaryl;
wherein R₄ₐ is H, C₁₋₆ alkyl, C₁₋₆ haloalkyl, —C(O)NR_{4b}R_{4c}, 3-6 membered cycloalkyl, 6-10 membered aryl optionally substituted with —OR_{4b}, —CN, 3-7 membered heterocyclyl, —(CH₂)ᵣOCH₃, or —(CH₂)ᵣOH, wherein r is 1, 2, or 3;
wherein each R_{4b} is independently H, C₁₋₆ alkyl; and
wherein each R₄c is independently H or C₁₋₆ alkyl; or
a therapeutically effective amount of Compound SOS1-(A) (also called RMC-0331), having the structure: 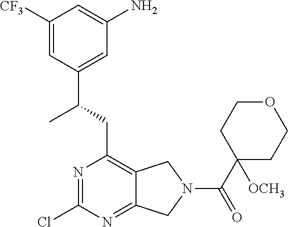 [SMILES: COC1(C(=O)N2Cc3nc(Cl)nc(C[C@H](C)c4cc(N)cc(C(F)(F)F)c4)c3C2)CCOCC1]
or a pharmaceutically acceptable salt, solvate, isomer, prodrug, or tautomer thereof, and
(b) a therapeutically effective amount of a RAS inhibitor selected from the group consisting of a RAS(ON) inhibitor and a RAS(OFF) inhibitor, and a combination thereof.

2. A method of treating a subject having a RAS protein-related disease or disorder, the method comprising administering to a subject in need of such treatment:
(a) a therapeutically effective amount of a SOS1 inhibitor, wherein the SOS1 inhibitor is BI-3406, having the structure: 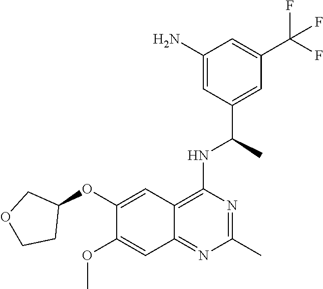 [SMILES: COc1cc2nc(C)nc(N[C@H](C)c3cc(N)cc(C(F)(F)F)c3)c2cc1O[C@H]1CCOC1]
or a pharmaceutically acceptable salt, solvate, isomer, prodrug, or tautomer thereof, and
(b) a therapeutically effective amount of a RAS inhibitor selected from the group consisting of a RAS(ON) inhibitor and a RAS(OFF) inhibitor, and a combination thereof.

3. A method of treating a subject having a RAS protein-related disease or disorder, the method comprising administering to a subject in need of such treatment:
(a) a therapeutically effective amount of a SOS1 inhibitor, wherein the SOS1 inhibitor is BI-1701963, or a pharmaceutically acceptable salt, solvate, isomer, prodrug, or tautomer thereof, and
(b) a therapeutically effective amount of a RAS inhibitor selected from the group consisting of a RAS(ON) inhibitor and a RAS(OFF) inhibitor, and a combination thereof.

4. The method of any one of claims 1 through 3, wherein the RAS inhibitor is selective for a mutation at position 12 or 13 of a RAS protein.

5. The method of any one of claims 1 through 4, wherein the RAS inhibitor is a RAS(ON) inhibitor.

6. The method of claim 5, wherein the RAS(ON) inhibitor is an inhibitor selective for RAS G12C, RAS G13D, or RAS G12D.

7. The method of claim 5, wherein the RAS(ON) inhibitor is a RAS(ON)^{MULTI} inhibitor.

8. The method of claim 5, wherein the RAS(ON) inhibitor is a compound described by Formula A00, Formula AI, Formula BI, Formula CI, Formula DIa, or subformula thereof, or a compound of Table A1, Table A2, Table B1, Table B2, Table C1, Table C2, Table D1a, Table D1b, Table D2, Table D3, or a pharmaceutically acceptable salt, solvate, hydrate, stereoisomer, or tautomer thereof.

9. The method of claim 8, wherein the RAS(ON) inhibitor is selected from a compound of Table A1 or Table A2, or a pharmaceutically acceptable salt thereof.

10. The method of claim 8, wherein the RAS(ON) inhibitor is selected from a compound of Table B1 or Table B2, or a pharmaceutically acceptable salt thereof.

11. The method of claim 8, wherein the RAS(ON) inhibitor is selected from a compound of Table C1 or Table C2, or a pharmaceutically acceptable salt thereof.

12. The method of claim 8, wherein the RAS(ON) inhibitor is selected from a compound of Table D1a, Table D1b, Table D2, Table D3, or a pharmaceutically acceptable salt thereof.

13. The method of claim 5, wherein the RAS(ON) inhibitor is selected from the group consisting of RAS-(A), RAS-(B), RAS-(C), RAS-(D), RAS-(E), RAS-(F), and any combination thereof.

14. The method of any one of claims 1 through 4, wherein the RAS inhibitor is a RAS(OFF) inhibitor.

15. The method of claim 14, wherein the RAS(OFF) inhibitor selectively targets RAS G12C.

16. The method of any one of claims 1 through 6, wherein the RAS inhibitor selectively targets RAS G12D.

17. The method of any one of claims 1 through 16, wherein the RAS inhibitor targets a wild-type RAS protein.

18. The method of any one of claims 1 through 17, wherein the RAS inhibitor targets a RAS protein mutation.

19. The method of claim 18, wherein the RAS protein mutation is at a position selected from the group consisting of G12, G13, Q61, A146, K117, L19, Q22, V14, A59, and a combination thereof.

20. The method of any one of claims 1 through 19, wherein the RAS inhibitor is a KRAS inhibitor and the RAS protein is KRAS.
